(19) United States
(12) Patent Application Publication
(10) Pub. No.: US 2024/0199546 A1
(43) Pub. Date: Jun. 20, 2024

(54) INHIBITORS OF SOLUTE CARRIER FAMILY 6A MEMBER 19 (SLC6A19) AND METHODS OF USE THEREOF

(71) Applicant: Maze Therapeutics, Inc., South San Francisco, CA (US)

(72) Inventors: Jennifer PITZEN, Oakland, CA (US); Maximiliano De La Higuera Macias, San Francisco, CA (US); Nicole Cooper, San Bruno, CA (US); Christopher Joseph Sinz, Walnut Creek, CA (US); Patrick Sang Tae Lee, Walnut Creek, CA (US); Jessica Wahlers, South San Francisco, CA (US); Nathan Fastman, Palo Alto, CA (US); Christos Tzitzilonis, Emeryville, CA (US); David John Morgans, JR., Los Altos, CA (US); Yuxi Liu, San Mateo, CA (US); Kevin Mellem, Redwood City, CA (US); Alexander Wayne Schammel, San Francisco, CA (US); Chris Ziebenhaus, San Francisco, CA (US); Adam Neil Reid, San Francisco, CA (US); Caleb Henry Karmel, San Francisco, CA (US)

(21) Appl. No.: 18/485,224

(22) Filed: Oct. 11, 2023

Related U.S. Application Data
(60) Provisional application No. 63/415,590, filed on Oct. 12, 2022.
(60) Provisional application No. 63/456,403, filed on Mar. 31, 2023.
(60) Provisional application No. 63/532,327, filed on Aug. 11, 2023.

(51) Int. Cl. C07D 209/20; A61P 43/00; C07D 401/12; C07D 403/06; C07D 403/12; C07D 403/14; C07D 409/12; C07D 413/06; C07D 413/12; C07D 413/14; C07D 417/06; C07D 417/12; C07D 417/14; C07D 471/04; C07D 493/08

(52) U.S. Cl. CPC C07D 209/20; A61P 43/00; C07D 401/12; C07D 403/06; C07D 403/12; C07D 403/14; C07D 409/12; C07D 413/06; C07D 413/12; C07D 413/14; C07D 417/06; C07D 417/12; C07D 417/14; C07D 471/04; C07D 493/08

(57) ABSTRACT

Provided herein are compounds of formula (II):

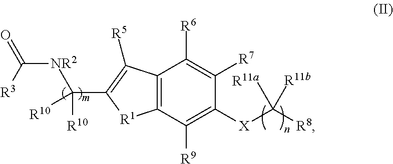

or a stereoisomer or tautomer thereof, or a pharmaceutically acceptable salt of any of the foregoing, wherein m, n, R², R³, R⁵, R⁶, R⁷, R⁸, R⁹, R¹⁰, R^{11a}, R^{11b}, and X are as defined elsewhere herein. Also provided herein are methods of preparing compounds of formula (II). Also provided herein are methods of inhibiting SLC6A19 and methods of treating a SLC6A19-mediated disease, disorder, or condition in an individual in need thereof.

### CROSS-REFERENCE TO RELATED APPLICATIONS

This application claims priority to U.S. Provisional Application No. 63/415,590 filed on Oct. 12, 2022, U.S. Provisional Application No. 63/456,403 filed on Mar. 31, 2023, and U.S. Provisional Application No. 63/532,327, filed Aug. 11, 2023, the contents of which are incorporated herein by reference in their entireties.

### BACKGROUND OF THE INVENTION

Solute carrier family 6A member 19 (SLC6A19), also known as B0AT1, is a sodium-dependent neutral amino acid (NAA) transporter that is predominantly expressed on the apical membranes of renal and intestinal epithelial cells (Fairweather et al. J. Biol. Chem (2015) 290, 24308-24325). In the intestines, surface expression of SLC6A19 is dependent on the chaperone protein ACE2, for amino acid uptake (Singer et al Am. J. Physiol. Gastrointest. Liver Physiol (2012) 303, G686-G695). In the kidneys, SLC6A19 requires TMEM27 for surface expression to reabsorb amino acids (Verrey et al. Arch. Eur. J. Physiol. (2009) 458, 53-60).

Phenylketonuria (PKU) is caused by mutations in the enzyme phenylalanine hydroxylase (PAH), the key enzyme in the metabolism of phenylalanine (Phe) to tyrosine (Tyr), both of which are NAA. PKU patients have toxic buildup of Phe in the blood and other tissues, leading to neurologic alterations (Scriver C. R Hum. Mutat. (2007) 28, 831-843). Treatment options for PKU include severely limiting protein intake, which has poor compliance, and the 2 FDA-approved drugs, Palynziq and Kuvan, both of which are limited to a subset of patients; significant unmet medical need still remain (Strisciuglio et. al. Metabolites (2014) 4, 1007-1017; Gentile et. al. Mol. Genet. and Metab. (2010) 99, S64-S67). Because the symptoms of PKU are a result of systemic buildup of Phe, inhibition of SLC6A19 to limit the intestinal absorption of Phe and its reuptake in the kidneys can be a viable approach to treating PKU. Indeed, loss of SLC6A19 in a mouse model of PKU significantly increased the excretion of Phe in urine and dramatically reduced plasma Phe and normalized neurotransmitter levels (Belanger et al. JCI Insight (2018) 3, e121762).

The prevalence of chronic kidney disease (CKD) is rapidly increasing and an estimated 15% of the US adult population have CKD (CDC 2021). An association of loss-of-function splice (c.1173+2T>G) (Sveinbjornsson G. et al. Hum. Mol. Genet. (2014) 23, 6935-6943) and missense (D173N) variants in SLC6A19 with protection from CKD has been reported (Sinnott-Amstrong N. et al. Nat. Genet. (2021) 53, 185-194).

Metabolic syndrome, and related diseases such as diabetes, is a global epidemic with an estimated one third of US adults having metabolic syndrome (Saklayen M. Curr Hypertens Rep (2018) 20, 12). Mice with deletion of SLC6A19 have a number of improved metabolic outcomes, including resistance to weight gain on a high-fat diet, improved glucose tolerance and insulin sensitivity, and increased energy expenditure (Jiang et al. Mol. Metab. (2015) 4, 406-417). These positive outcomes are likely due to increased secretion of FGF21 and GLP-1, two hormones that play important roles in regulating energy metabolism, (Geng et al. Nat Rev Endo. (2020) 16, 654-667; Baggio et al. Mol Metab (2021) 46). Inhibiting SLC6A19, therefore, may be an effective approach to treat metabolic diseases.

In humans, SLC6A19 biallelic loss of function results in a rare disease called Hartnup disorder, which is predominantly asymptomatic in patients with adequate nutrition (Seow et al. Nat. Genet. (2004) 36, 1003-1007. Azmanov et al. Hum. Mutat. (2008) 29, 1217-1221). Since the NAA tryptophan is essential for the synthesis of nicotinamide, Hartnup patients may develop niacin deficiency and its associated symptoms, which include dermatitis, photosensitivity, and psychosis, but these symptoms are well-controlled with niacin supplementation (Hashmi et al. StatPearls (2021)). Similarly, mice with complete loss of SLC6A19 are viable and fertile (Jiang et al. Mol. Metab. (2015) 4, 406-417). Thus, inhibition of SLC6A19 is believed to be a viable therapeutic approach.

There remains a need for therapies to treat such diseases/disorders.

### BRIEF SUMMARY OF THE INVENTION

In one aspect, provided herein is a compound of formula (II):

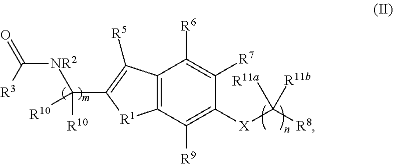

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₁₋₆alkoxy, —C(O)C₁₋₆alkyl, C₃₋₁₀cycloalkyl, —N(R⁴)₂, 5-20 membered heteroaryl, —C₁₋₆alkyl(5-20 membered heteroaryl), or 4-10 membered heterocyclyl, wherein each R³ is optionally substituted with one or more R^{a}, and wherein when R³ is 6-10 membered heteroaryl, the 6-10 membered heteroaryl is unsubstituted;
each R⁴ is independently H, C₁₋₆alkyl, C₁₋₆haloalkyl, —C₁₋₆alkyl(5-20 membered heteroaryl), or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₁₋₆thioalkyl, C₃₋₁₀cycloalkyl, C₁₋₆haloalkyl, or 5-20 membered heteroaryl, wherein
   the C₁₋₆alkyl, C₁₋₆alkoxy, or C₃₋₁₀cycloalkyl of R⁵ and R⁷ are each independently optionally substituted with one or more halo or CN;
R⁶ and R⁹ are each independently H or halo;
R⁸ is —C(O)N(R⁴)₂, C₁₋₆alkoxy, C₃₋₁₀cycloalkyl, —O—C₃₋₁₀cycloalkyl, C₆₋₂₀aryl, 3-10 membered heterocyclyl, 5-20 membered heteroaryl, or —(R^{x})-5-20 membered heteroaryl), wherein each R⁸ is optionally substituted with one or more R^{a};
R^{x} is —NH— or —O—;
R¹⁰ is, independently at each occurrence, H or D;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently D, —OH, oxo, —CN, halo, C₁₋₆ alkyl, C₁₋₆alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆ alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl of R^{a} is optionally substituted with one or more R^{b}; and
each R^{b} is independently D, —OH, halo, C₁₋₆alkyl, or C₁₋₆ alkoxy.

In one aspect, provided herein is a compound of formula (I′):

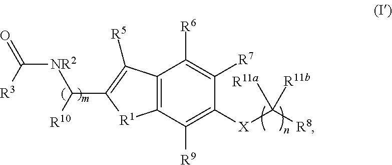

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆ alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
   the C₁₋₆ alkyl of R⁵ and R⁷ are each independently optionally substituted with one or more halo or CN;
R⁶ and R⁹ are each independently H or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy.

In one aspect, provided herein is a compound of formula (I):

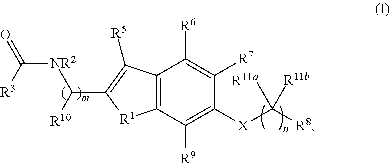

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with one of R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl. Any embodiments provided herein of a compound of formula (I), or a stereoisomer or tautomer thereof, or a pharmaceutically acceptable salt of any of the foregoing, or any variation or embodiment thereof, are also embodiments of a compound of formula (I′), or a stereoisomer or tautomer thereof, or a pharmaceutically acceptable salt of any of the foregoing, or any variation or embodiment thereof.

In one aspect, provided herein is a compound of formula (I-A):

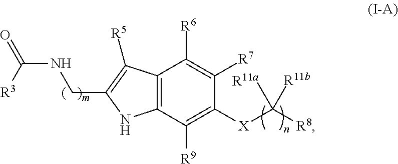

or a stereoisomer or tautomer thereof, or a pharmaceutically acceptable salt of any of the foregoing, wherein m, n, R³, R⁵, R⁶, R⁷, R⁸, R⁹, R^{11a}, R^{11b}, and X are as defined elsewhere herein. In another variation, m, n, R³, R⁵, R⁶, R⁷, R⁸, R⁹, R^{11a}, R^{11b}, and X of formula (I-A) are as defined for a compound of formula (I′), or (II), or a stereoisomer or tautomer thereof, or a pharmaceutically acceptable salt of any of the foregoing, or any variation or embodiment thereof.

In one aspect, provided herein is a compound of formula (I-B):

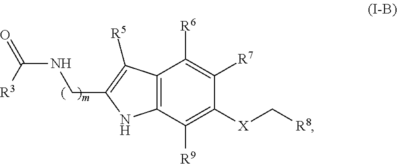

or a stereoisomer or tautomer thereof, or a pharmaceutically acceptable salt of any of the foregoing, wherein m, R³, R⁵, R⁶, R⁷, R⁸, R⁹, and X are as defined elsewhere herein. In another variation, m, R³, R⁵, R⁶, R⁷, R⁸, R⁹, and X of formula (I-B) are as defined for a compound of formula (I′), or (II) or a stereoisomer or tautomer thereof, or a pharmaceutically acceptable salt of any of the foregoing, or any variation or embodiment thereof.

In one aspect, provided herein is a compound of formula (I-C):

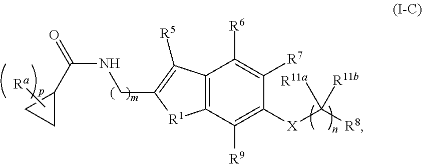

or a stereoisomer or tautomer thereof, or a pharmaceutically acceptable salt of any of the foregoing, wherein m, n, p, R^{a}, R⁵, R⁶, R⁷, R⁸, R⁹, R^{11a}, R^{11b}, and X are as defined elsewhere herein. In another variation, m, n, p, R^{a}, R⁵, R⁶, R⁷, R⁸, R⁹, R^{11a}, R^{11b}, and X of formula (I-C) are as defined for a compound of formula (I′), or (II) or a stereoisomer or tautomer thereof, or a pharmaceutically acceptable salt of any of the foregoing, or any variation or embodiment thereof.

In one aspect, provided herein is a compound of formula (I-D):

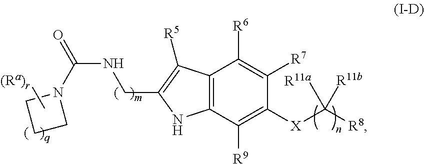

or a stereoisomer or tautomer thereof, or a pharmaceutically acceptable salt of any of the foregoing, wherein m, n, q, r, R^{a}, R⁵, R⁶, R⁷, R⁸, R⁹, R^{11a}, R^{11b}, and X are as defined elsewhere herein. In another variation, m, n, q, r, R^{a}, R⁵, R⁶, R⁷, R⁸, R⁹, R^{11a}, R^{11b}, and X of formula (I-D) are as defined for a compound of formula (I′), or (II) or a stereoisomer or tautomer thereof, or a pharmaceutically acceptable salt of any of the foregoing, or any variation or embodiment thereof.

In one aspect, provided is a compound of formula (I-E):

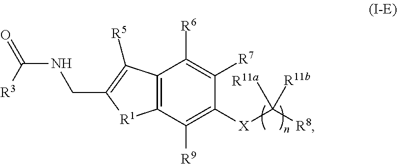

or a stereoisomer or tautomer thereof, or a pharmaceutically acceptable salt of any of the foregoing, wherein n, R^{a}, R¹, R³, R⁵, R⁶, R⁷, R⁸, R⁹, R^{11a}, R^{11b}, and X are as defined elsewhere herein. In another variation, n, R^{a}, R¹, R³, R⁵, R⁶, R⁷, R⁸, R⁹, R^{11a}, R^{11b}, and X of formula (I-E) are as defined for a compound of formula (I′), or (II) or a stereoisomer or tautomer thereof, or a pharmaceutically acceptable salt of any of the foregoing, or any variation or embodiment thereof.

In one aspect, provided herein is a compound of formula (I-F):

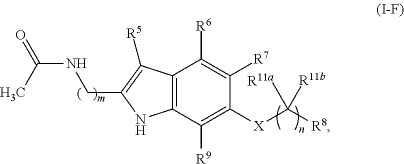

or a stereoisomer or tautomer thereof, or a pharmaceutically acceptable salt of any of the foregoing, wherein m, n, R⁵, R⁶, R⁷, R⁸, R⁹, R^{11a}, R^{11b}, and X are as defined elsewhere herein. In another variation, m, n, R⁵, R⁶, R⁷, R⁸, R⁹, R^{11a}, R^{11b}, and X of formula (I-F) are as defined for a compound of formula (I′), or (II) or a stereoisomer or tautomer thereof, or a pharmaceutically acceptable salt of any of the foregoing, or any variation or embodiment thereof.

In one aspect, provided herein is a compound of formula (I-G):

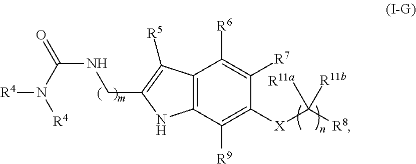

or a stereoisomer or tautomer thereof, or a pharmaceutically acceptable salt of any of the foregoing, wherein m, n, each R⁴, R⁵, R⁶, R⁷, R⁸, R⁹, R^{11a}, R^{11b}, and X are as defined elsewhere herein. In another variation, m, n, each R⁴, R⁵, R⁶, R⁷, R⁸, R⁹, R^{11a}, R^{11b}, and X of formula (I-G) are as defined for a compound of formula (I′), or (II) or a stereoisomer or tautomer thereof, or a pharmaceutically acceptable salt of any of the foregoing, or any variation or embodiment thereof.

In one aspect, provided herein is a compound of formula (I-H):

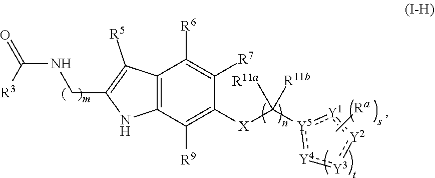

or a stereoisomer or tautomer thereof, or a pharmaceutically acceptable salt of any of the foregoing, wherein m, n, s, t, R³, R⁵, R⁶, R⁷, R⁹, R^{11a}, R^{11b}, R^{a}, X, Y¹, Y², Y³, Y⁴, and Y⁵ are as defined elsewhere herein. In another variation, m, n, s, t, R³, R⁵, R⁶, R⁷, R⁹, R^{11a}, R^{11b}, R^{a}, X, Y, Y², Y³, Y⁴, and Y⁵ of formula (I-H) are as defined for a compound of formula (I′), or (II) or a stereoisomer or tautomer thereof, or a pharmaceutically acceptable salt of any of the foregoing, or any variation or embodiment thereof.

Any embodiments provided herein of a compound of formula (I) or (I′), or a stereoisomer or tautomer thereof, or a pharmaceutically acceptable salt of any of the foregoing, apply where applicable to any other formula detailed herein, the same as if each and every embodiment were specifically and individually listed. Thus, it is understood and described that each embodiment provided herein of a compound of formula (I) or (I′), or a stereoisomer or tautomer thereof, or a pharmaceutically acceptable salt of any of the foregoing, such as embodiments related to m, n, R², R³, R⁵, R⁶, R⁷, R⁸, R⁹, R¹⁰, R^{11a}, R^{11b} and X apply to formula (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, the same as if each and every embodiment were specifically and individually listed. It is also understood and described that all such embodiments may be used in any of the pharmaceutical compositions, methods, kits, uses, or other aspects detailed herein.

In one aspect, provided herein is a pharmaceutical composition, comprising (i) a compound of formula (I), or any variation or embodiment thereof, or a stereoisomer or tautomer thereof, or a pharmaceutically acceptable salt of any of the foregoing, and (ii) one or more pharmaceutically acceptable excipients. In another variation, provided herein is a pharmaceutical composition, comprising (i) a compound of formula (I′), or any variation or embodiment thereof, or a stereoisomer or tautomer thereof, or a pharmaceutically acceptable salt of any of the foregoing, and (ii) one or more pharmaceutically acceptable excipients. This aspect in some embodiments may employ a compound of any of formulas (II), (I′), (I), (I-A), (I-A1), (I-A2), (I-A3), (I-A4), (I-A5), (I-A6), (I-B), (I-B31), (I-B32), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of modulating SLC6A19 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of modulating SLC6A19 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In another variation, provided herein is a method of modulating SLC6A19 in a cell, comprising exposing the cell to an effective amount of (i) a compound of formula (I′), or any variation or embodiment thereof, or a stereoisomer or tautomer thereof, or a pharmaceutically acceptable salt of any of the foregoing, or (ii) a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients. In some embodiments, modulating SLC6A19 comprises inhibition of SLC6A19. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of inhibiting SLC6A19 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of inhibiting SLC6A19 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In another variation, provided herein is a method of inhibiting SLC6A19 in a cell, comprising exposing the cell to an effective amount of (i) a compound of formula (I′), or any variation or embodiment thereof, or a stereoisomer or tautomer thereof, or a pharmaceutically acceptable salt of any of the foregoing, or (ii) a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients.

This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of modulating SLC6A19 in a cell of an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, modulating SLC6A19 comprises inhibition of SLC6A19. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of modulating SLC6A19 in a cell of an individual in need thereof, comprising administering to the individual an effective amount of (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In another variation, provided herein is a method of modulating SLC6A19 in a cell of an individual in need thereof, comprising administering to the individual an effective amount of (i) a compound of formula (I′), or any variation or embodiment thereof, or a stereoisomer or tautomer thereof, or a pharmaceutically acceptable salt of any of the foregoing, or (ii) a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients. In some embodiments, modulating SLC6A19 comprises inhibition of SLC6A19. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of treating a SLC6A19-mediated disease, disorder, or condition in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the disease, disorder, or condition is responsive to SLC6A19 inhibition. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a method of treating a SLC6A19-mediated disease, disorder, or condition in an individual in need thereof, comprising administering to the individual an effective amount of (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In another variation, provided herein is a method of treating a SLC6A19-mediated disease, disorder, or condition in an individual in need thereof, comprising administering to the individual an effective amount of (i) a compound of formula (I′), or any variation or embodiment thereof, or a stereoisomer or tautomer thereof, or a pharmaceutically acceptable salt of any of the foregoing, or (ii) a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients. In some embodiments, the disease, disorder, or condition is responsive to SLC6A19 inhibition. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In another variation, provided herein is a method of treating a SLC6A19-mediated disease, disorder, or condition in an individual in need thereof, comprising administering to the individual (i) a compound of formula (I′), or any variation or embodiment thereof, or a stereoisomer or tautomer thereof, or a pharmaceutically acceptable salt of any of the foregoing, or (ii) a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients. In some embodiments, a therapeutically effective amount of the compound or pharmaceutical composition is administered. In some embodiments, the disease, disorder, or condition is responsive to SLC6A19 inhibition. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a kit, comprising (i) a composition comprising an effective amount of a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an SLC6A19-mediated disease, disorder, or condition in an individual in need thereof. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In one aspect, provided herein is a kit, comprising (i) an effective amount of a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an SLC6A19-mediated disease, disorder, or condition in an individual in need thereof. In another variation, provided herein is a kit, comprising an effective amount of (i) a compound of formula (I′), or any variation or embodiment thereof, or a stereoisomer or tautomer thereof, or a pharmaceutically acceptable salt of any of the foregoing, or a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients, and (ii) instructions for use in treating an SLC6A19-mediated disease, disorder, or condition in an individual in need thereof. This aspect in some embodiments may employ a compound of any of formulas (II), (I′), (I), (I-A), (I-A1), (I-A2), (I-A3), (I-A4), (I-A5), (I-A6), (I-B), (I-B31), (I-B32), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In another variation, provided herein is a kit, comprising (i) a compound of formula (I′), or any variation or embodiment thereof, or a stereoisomer or tautomer thereof, or a pharmaceutically acceptable salt of any of the foregoing, or a pharmaceutical composition, comprising a compound of formula (I′), or any variation or embodiment thereof, or a stereoisomer or tautomer thereof, or a pharmaceutically acceptable salt of any of the foregoing, and one or more pharmaceutically acceptable excipients, and (ii) instructions for use in treating an SLC6A19-mediated disease, disorder, or condition in an individual in need thereof. In some embodiments, the kit comprises a therapeutically effective amount of the compound or pharmaceutical composition. This aspect in some embodiments may employ a compound of any of formulas (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In some aspect, provided herein are methods of preparing a compound of formula (I), or any embodiment or variation thereof, such as a compound of formula (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing. In another variation, provided herein are methods of preparing a compound of formula (I′), or any embodiment or variation thereof, such as a compound of formula (II), (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

All pharmaceutical compositions, methods, kits, uses, or other aspects described herein with reference to formula (I) or (I′), or a pharmaceutically acceptable salt of any of the foregoing, are also hereby described and embraced for any one of the other formulas detailed herein such as formula (II), the same as if each and every embodiment were specifically and individually listed.

### DETAILED DESCRIPTION OF THE INVENTION

“Individual” refers to mammals and includes humans and non-human mammals. Examples of individuals include, but are not limited to, mice, rats, hamsters, guinea pigs, pigs, rabbits, cats, dogs, goats, sheep, cows, and humans. In some embodiments, individual refers to a human.

As used herein, “about” a parameter or value includes and describes that parameter or value per se. For example, “about X” includes and describes X per se.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition; slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated univalent hydrocarbon chain. As used herein, alkyl has 1-20 carbons (i.e., C₁₋₂₀alkyl), 1-16 carbons (i.e., C₁₋₁₆alkyl), 1-12 carbons (i.e., C₁₋₁₂alkyl), 1-10 carbons (i.e., C₁₋₁₀alkyl), 1-8 carbons (i.e., C₁₋₈alkyl), 1-6 carbons (i.e., C₁₋₆alkyl), 1-4 carbons (i.e., C₁₋₄alkyl), or 1-3 carbons (i.e., C₁₋₃alkyl). Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkenyl”, as used herein, refers to a branched or unbranched univalent hydrocarbon chain comprising at least one carbon-carbon double bond. As used herein, alkenyl has 2-20 carbons (i.e., C₂₋₂₀alkenyl), 2-16 carbons (i.e., C₂₋₁₆alkenyl), 2-12 carbons (i.e., C₂₋₁₂alkenyl), 2-10 carbons (i.e., C₂₋₁₀alkenyl), 2-8 carbons (i.e., C₂₋₈alkenyl), 2-6 carbons (i.e., C₂₋₆alkenyl), 2-4 carbons (i.e., C₂₋₄alkenyl), or 2-3 carbons (i.e., C₂₋₃alkenyl). Examples of alkenyl include, but are not limited to, ethenyl, prop-1-enyl, prop-2-enyl 1,2-butadienyl, and 1,3-butadienyl. When an alkenyl residue having a specific number of carbons is named by chemical name or molecular formula, all positional isomers having that number of carbon atoms may be encompassed—for example, “propenyl” includes prop-1-enyl and prop-2-enyl. Certain commonly used alternative names may be used and will be understood by those of ordinary skill in the art. For instance, a divalent group, such as a divalent “alkenyl” group, may be referred to as an “alkenylene”.

The term “alkynyl”, as used herein, refers to a branched or unbranched univalent hydrocarbon chain comprising at least one carbon-carbon triple bond. As used herein, alkynyl has 2-20 carbons (i.e., C₂₋₂₀alkynyl), 2-16 carbons (i.e., C₂₋₁₆alkynyl), 2-12 carbons (i.e., C₂₋₁₂alkynyl), 2-10 carbons (i.e., C₂₋₁₀alkynyl), 2-8 carbons (i.e., C₂₋₈alkynyl), 2-6 carbons (i.e., C₂₋₆alkynyl), 2-4 carbons (i.e., C₂₋₄alkynyl), or 2-3 carbons (i.e., C₂₋₃alkynyl). Examples of alkynyl include, but are not limited to, ethynyl, prop-1-ynyl, prop-2-ynyl, but-1-ynyl, but-2-ynyl, and but-3-ynyl. When an alkynyl residue having a specific number of carbons is named by chemical name or molecular formula, all positional isomers having that number of carbon atoms may be encompassed—for example, “propynyl” includes prop-1-ynyl and prop-2-ynyl. Certain commonly used alternative names may be used and will be understood by those of ordinary skill in the art. For instance, a divalent group, such as a divalent “alkynyl” group, may be referred to as an “alkynylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “thioalkyl”, as used herein refers to the groups —S-alkyl.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond. Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbonyl, decalinyl, 7,7-dimethyl-bicyclo [2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “halo”, as used herein, refers to atoms occupying groups VIIA of The Periodic Table and includes fluorine (fluoro), chlorine (chloro), bromine (bromo), and iodine (iodo). Additionally, terms such as “haloalkyl” are meant to include monohaloalkyl and polyhaloalkyl. For example, the term “C₁-C₄ haloalkyl” is mean to include trifluoromethyl, 2,2,2-trifluoroethyl, 4-chlorobutyl, 3-bromopropyl, difluoromethyl, and the like.

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The term “oxo”, as used herein, refers to a ═O moiety.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In some embodiments, a compound of formula (I′) or (I) is provided wherein one or more hydrogen is replaced by deuterium or tritium.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vitro or in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

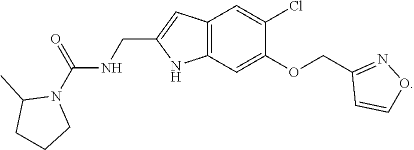 [SMILES: CC1CCCN1C(=O)NCc1cc2cc(Cl)c(OCc3ccon3)cc2[nH]1]

Where enantiomeric and/or diastereomeric forms exist of a given structure with two stereocenters, wedged and/or dashed bonds and the presence of two “&1” symbols indicate the composition is made up of a pair of enantiomers with known relative stereochemistry, e.g.,

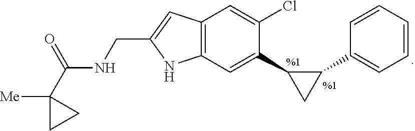 [SMILES: CC1(C(=O)NCc2cc3cc(Cl)c([C@@H]4C[C@H]4c4ccccc4)cc3[nH]2)CC1]

### Compounds

In one aspect, provided herein is a compound of formula (II):

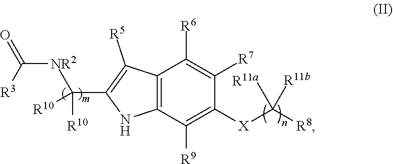

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₁₋₆alkoxy, —C(O)C₁₋₆alkyl, C₃₋₁₀cycloalkyl, —N(R⁴)₂, 5-20 membered heteroaryl, —C₁₋₆alkyl(5-20 membered heteroaryl), or 4-10 membered heterocyclyl, wherein each R³ is optionally substituted with one or more R^{a}, and wherein when R³ is 6-10 membered heteroaryl, the 6-10 membered heteroaryl is unsubstituted;
each R⁴ is independently H, C₁₋₆alkyl, C₁₋₆haloalkyl, —C₁₋₆alkyl(5-20 membered heteroaryl), or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₁₋₆thioalkyl, C₃₋₁₀cycloalkyl, C₁₋₆haloalkyl, or 5-20 membered heteroaryl, wherein
   the C₁₋₆alkyl, C₁₋₆alkoxy, or C₃₋₁₀cycloalkyl of R⁵ and R⁷ are each independently optionally substituted with one or more halo or CN;
R⁶ and R⁹ are each independently H or halo;
R⁸ is —C(O)N(R⁴)₂, C₁₋₆alkoxy, C₃₋₁₀cycloalkyl, —O—C₃₋₁₀cycloalkyl, C₆₋₂₀aryl, 3-10 membered heterocyclyl, 5-20 membered heteroaryl, or —(R^{x})-5-20 membered heteroaryl), wherein each R⁸ is optionally substituted with one or more R^{a};
R^{x} is —NH— or —O—;
R¹⁰ is, independently at each occurrence, H or D;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently D, —OH, oxo, —CN, halo, C₁₋₆ alkyl, C₁₋₆alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl of R^{a} is optionally substituted with one or more R^{b}; and
each R^{b} is independently D, —OH, halo, C₁₋₆ alkyl, or C₁₋₆ alkoxy.

In one aspect, provided herein is a compound of formula (I′):

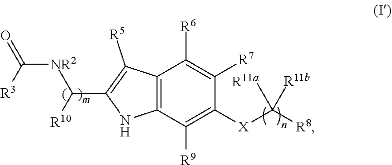

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆ alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
   the C₁₋₆ alkyl of R⁵ and R⁷ are each independently optionally substituted with one or more halo, or CN;
R⁶ and R⁹ are each independently H or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H, or C₁₋₆alkyl, and the other of R^{12a} and R^{12b} is taken together with one of R^{11a} or R^{11b} to form a C₃₋₁₀cycloalkyl;
each R^{a} is, independently at each occurrence, —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C_{1- 6}alkyl)₂, —C(O)NH₂, —C(O)NH(C₁₋₆alkyl), C(O)N(C₁₋₆ alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl or C₃₋₁₀cycloalkyl of R^{a} is optionally substituted with one or more R^{b}; and
each R^{b} is, independently at each occurrence, D, —OH, halo, C₁₋₆ alkyl or C₁₋₆ alkoxy.

In one aspect, provided herein is a compound of formula (I):

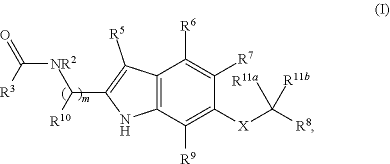

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₃₋₁₀cycloalkyl or C₁₋₆ haloalkyl, wherein
the C₁₋₆alkyl of R⁵ and R⁷ are each independently optionally substituted with one or more halo, or CN;
R⁶ and R⁹ are each independently H, or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆ alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with one of R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl.

Any embodiments provided herein of a compound of formula (I), or a stereoisomer or tautomer thereof, or a pharmaceutically acceptable salt of any of the foregoing, or any variation or embodiment thereof, are also embodiments of a compound of formula (I′), or a stereoisomer or tautomer thereof, or a pharmaceutically acceptable salt of any of the foregoing, or any variation or embodiment thereof. Any embodiments provided herein of a compound of formula (I) or (I′), or a stereoisomer or tautomer thereof, or a pharmaceutically acceptable salt of any of the foregoing, or any variation or embodiment thereof, are also embodiments of a compound of formula (II), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

Any embodiments provided herein of a compound of formula (I) or (I′), or a stereoisomer or tautomer thereof, or a pharmaceutically acceptable salt of any of the foregoing, apply where applicable to any other formula detailed herein, the same as if each and every embodiment were specifically and individually listed. Thus, it is understood and described that each embodiment provided herein of a compound of formula (I) or (I′), or a stereoisomer or tautomer thereof, or a pharmaceutically acceptable salt of any of the foregoing, such as embodiments related to m, n, R², R³, R⁵, R⁶, R⁷, R⁸, R⁹, R¹⁰, R^{11a}, R^{11b}, and X apply to formula (II), the same as if each and every embodiment were specifically and individually listed. It is also understood and described that all such embodiments may be used in any of the pharmaceutical compositions, methods, kits, uses, or other aspects detailed herein. And, each embodiment provided herein of a compound of formula (I) or (I′), or a stereoisomer or tautomer thereof, or a pharmaceutically acceptable salt of any of the foregoing, such as embodiments related to m, n, R², R³, R⁵, R⁶, R⁷, R⁸, R⁹, R¹⁰, R^{11a}, R^{11b}, and X also apply to formula (I-A), (I-A1), (I-A2), (I-A3), (I-A4), (I-A5), (I-A6), (I-B), (I-B1), (I-B2), (I-B3), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

In some embodiments of a compound of formula (II), or a stereoisomer or tautomer thereof, or a pharmaceutically acceptable salt of any of the foregoing, the compound may comprise one or more of any of the following structural features:
(a) m is 1; and R¹ is —NH— or —N(C₁₋₆alkyl);
(b) X is —O—; n is an integer from 0 to 2; and R⁸ is —C(O)N(R⁴)₂, C₁₋₆alkoxy, C₃₋₁₀cycloalkyl, —O—C₃₋₁₀cycloalkyl, C₆₋₂₀aryl, 3-10 membered heterocyclyl, 5-20 membered heteroaryl, wherein each R⁸ is optionally substituted with one or more R^{a};
(c) X is —N(H)—, or —N(C₁₋₆alkyl)-; n is 1; and R⁸ is C₆₋₂₀aryl, or 5-20 membered heteroaryl, wherein each R⁸ is optionally substituted with one or more R^{a};
(d) X is —C(R^{12a})(R^{12b})—; n is 0 or 1; R⁸ is C₆₋₂₀aryl, 5-20 membered heteroaryl, or —(R^{x})-5-20 membered heteroaryl), wherein each R⁸ is optionally substituted with one or more R^{a}; and R^{x} is —NH— or —O—;
(e) X is —S—; n is 1; and R⁸ is 5-20 membered heteroaryl;
(f) R³ is C₁₋₆alkyl, C₁₋₆alkoxy, —C(O)C₁₋₆alkyl, C₃₋₁₀cycloalkyl, —N(R⁴)₂, 5-20 membered heteroaryl, —C₁₋₆alkyl(5-20 membered heteroaryl), or 4-10 membered heterocyclyl, wherein each R³ is optionally substituted with one or more R^{a}, and wherein when R³ is 6-10 membered heteroaryl, the 6-10 membered heteroaryl is unsubstituted;
(g) R³ is C₁₋₆alkyl, C₃₋₁₀cycloalkyl, or 4-10 membered heterocyclyl, wherein each R³ is optionally substituted with one or more R^{a};
(h) R³ is, C₃₋₁₀cycloalkyl, or 4-10 membered heterocyclyl, wherein each R³ is optionally substituted with one or more R^{a}; and/or
(i) R³ is C₃₋₁₀cycloalkyl optionally substituted with one or more R^{a}.
   In some embodiments of a compound of formula (II), or a stereoisomer or tautomer thereof, or a pharmaceutically acceptable salt of any of the foregoing, the compound comprises (a). In some embodiments, the compound comprises (a), (b), and (f). In some embodiments, the compound comprises (a), (c), and (g). In some embodiments, the compound comprises (a), (d), and (h). In some embodiments, the compound comprises (a), (e), and (i).

In some embodiments of a compound of formula (I), or a stereoisomer or tautomer thereof, or a pharmaceutically acceptable salt of any of the foregoing,
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
the C₁₋₆alkyl of R⁵ and R⁷ are each independently optionally substituted with one or more halo, or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with one of R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or a stereoisomer or tautomer thereof, or a pharmaceutically acceptable salt of any of the foregoing,
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl, or C₁₋₆haloalkyl, wherein
   the C₁₋₆alkyl of R⁵ and R⁷ are each independently optionally substituted with one or more halo, or CN;
R⁶ and R⁹ are each independently H or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with one of R^{11a} or R^{11b} to form a C₃₋₁₀cycloalkyl;
each R^{a} is, independently at each occurrence, —OH, oxo, —CN, halo, C₁₋₆ alkyl, C₁₋₆ alkoxy, C₁₋₆haloalkyl, —O—C₁₋₆ haloalkyl, —NH₂, —NH(C₁₋₆alkyl), —N(C₁₋₆ alkyl)₂, 4-10 membered heterocyclyl, or C₃₋₁₀cycloalkyl, wherein
   the C₁₋₆ alkyl, C₁₋₆ alkoxy, 4-10 membered heterocyclyl or C₃₋₁₀cycloalkyl of R^{a} is optionally substituted with one or more R^{b}; and
each R^{b} is, independently at each occurrence, —OH, halo, C₁₋₆alkyl or C₁₋₆alkoxy. In some embodiments of a compound of formula (I), or a stereoisomer or tautomer thereof, or a pharmaceutically acceptable salt of any of the foregoing,
m is an integer from 1 to 2;
n is an integer from 0 to 2;
X is —O—, —S—, —C(R^{12a})(R^{12b})—, —N(H)—, or —N(C₁₋₆ alkyl)-;
R¹ is —NH—;
R² is H;
R³ is C₁₋₃alkyl, C₁₋₃haloalkyl, C₃₋₆cycloalkyl, —N(R⁴)₂, or 4-6 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-6 membered heterocyclyl optionally substituted with one or more R^{a};
R⁵ and R⁷ are each independently H, halo, C₁₋₃alkyl, C₃₋₆cycloalkyl or C₁₋₃haloalkyl, wherein
the C₁₋₃alkyl of R⁵ and R⁷ are each independently optionally substituted with one or more halo, or CN;
R⁶ and R⁹ are each independently H, or halo;
R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a};
R¹⁰ is H;
R^{11a} and R^{11b} are each independently at each occurrence, H, or C₁₋₃alkyl, or taken together with one of R^{12a} or R^{12b} to form a C₃₋₆cycloalkyl;
R^{12a} and R^{12b} are each independently, H, or C₁₋₃alkyl, or
one of R^{12a} and R^{12b} is H, or C₁₋₆alkyl, and the other of R^{12a} and R^{12b} is taken together with one of R^{11a} or R^{11b} to form a C₃₋₆cycloalkyl; and
each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or a stereoisomer or tautomer thereof, or a pharmaceutically acceptable salt of any of the foregoing,
m is an integer from 1 to 2;
n is an integer from 0 to 2;
X is —O—, —S—, —C(R^{12a})(R^{12b})—, —N(H)—, or —N(C₁₋₆alkyl)-;
R¹ is —NH—;
R² is H;
R³ is C₁₋₃alkyl, C₁₋₃haloalkyl, C₃₋₆cycloalkyl, —N(R⁴)₂, or 4-6 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-6 membered heterocyclyl optionally substituted with one or more R^{a};
R⁵ and R⁷ are each independently H, halo, C₁₋₃alkyl, C₃₋₆cycloalkyl, or C₁₋₃haloalkyl, wherein
the C₁₋₃alkyl of R⁵ and R⁷ are each independently optionally substituted with one or more halo, or CN;
R⁶ and R⁹ are each independently H or halo;
R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a};
R¹⁰ is H;
R^{11a} and R^{11b} are each independently at each occurrence, H or C₁₋₃alkyl, or
one of R^{1a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₃alkyl, or
one of R^{12a} and R^{12b} is H or C₁₋₆alkyl, and the other of R^{12a} and R^{12b} is taken together with one of R^{11a} or R^{11b} to form a C₃₋₆cycloalkyl;
each R^{a} is, independently at each occurrence, —OH, oxo, —CN, halo, C₁₋₆ alkyl, C₁₋₆ alkoxy, C₁₋₆haloalkyl, —O—C₁₋₆ haloalkyl, —NH₂, —NH(C₁₋₆alkyl), —N(C₁₋₆ alkyl)₂, 4-10 membered heterocyclyl, or C₃₋₁₀cycloalkyl, wherein
the C₁₋₆ alkyl, C₁₋₆ alkoxy, 4-10 membered heterocyclyl or C₃₋₁₀cycloalkyl of R^{a} is optionally substituted with one or more R^{b}; and
each R^{b} is, independently at each occurrence, —OH, halo, C₁₋₆alkyl or C₁₋₆ alkoxy.

In some embodiments of a compound of formula (I), or a stereoisomer or tautomer thereof, or a pharmaceutically acceptable salt of any of the foregoing, m is an integer from 1 to 4; n is an integer from 0 to 4; X is —N(H)—; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀cycloalkyl or C₁₋₆ haloalkyl, wherein the C₁₋₆ alkyl of R⁵ and R⁷ are each independently optionally substituted with one or more halo, or CN; R⁶ and R⁹ are each independently H, or halo; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11a} and R^{11b} are each independently at each occurrence, H, or C₁₋₆alkyl, or taken together with one of R^{12a} or R^{12b} to form a C₃₋₁₀cycloalkyl; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11a} or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is an integer from 0 to 2; X is —N(H)—; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11a} and R^{11b} are each independently at each occurrence, H, or C₁₋₃alkyl, or taken together with one of R^{12a} or R^{12b} to form a C₃₋₆cycloalkyl; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or a stereoisomer or tautomer thereof, or a pharmaceutically acceptable salt of any of the foregoing, m is 1; n is an integer from 0 to 4; X is —O—, —S—, —C(R^{12a})(R^{12b})—, —N(H)—, or —N(C₁₋₆alkyl)-; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11a} or R^{11b} to form a C₃₋₁₀cycloalkyl; each R^{a} is, independently at each occurrence, —OH, oxo, —CN, halo, C₁₋₆ alkyl, C₁₋₆ alkoxy, C₁₋₆ haloalkyl, —O—C₁₋₆ haloalkyl, —NH₂, —NH(C₁₋₆alkyl), —N(C₁₋₆ alkyl)₂, 4-10 membered heterocyclyl, or C₃₋₁₀cycloalkyl, wherein the C₁₋₆alkyl, C₁₋₆alkoxy, 4-10 membered heterocyclyl or C₃₋₁₀cycloalkyl of R^{a} is optionally substituted with one or more R^{b}; and each R^{b} is, independently at each occurrence, —OH, halo, C₁₋₆alkyl or C₁₋₆ alkoxy. In some embodiments, m is an integer from 1 to 2; n is 1; X is —O—, —S—, —C(R^{12a})(R^{12b})—, —N(H)—, or —N(C₁₋₆ alkyl)-; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₃alkyl, or one of R^{12a} and R^{12b} is H, or C₁₋₃alkyl, and the other of R^{12a} and R^{12b} is taken together with one of R^{11b}a or R^{11b} to form a C₃₋₆cycloalkyl; each R^{a} is, independently at each occurrence, —OH, oxo, —CN, halo, C₁₋₃alkyl, C₁₋₃alkoxy, C₁₋₃haloalkyl, —O—C₁₋₃haloalkyl, —NH₂, —NH(C₁₋₃alkyl), —N(C₁₋₃alkyl)₂, 4-6 membered heterocyclyl, or C₃₋₆cycloalkyl, wherein the C₁₋₆ alkyl, C₁₋₆ alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy.

In some embodiments of a compound of formula (I), or a stereoisomer or tautomer thereof, or a pharmaceutically acceptable salt of any of the foregoing, m is 1; n is an integer from 0 to 4; X is —O—, —S—, —C(R^{12a})(R^{12b})—, —N(H)—, or —N(C₁₋₆alkyl)-; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11a} or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is 1; X is —O—, —S—, —C(R^{12a})(R^{12b})—, —N(H)—, or —N(C₁₋₆alkyl)-; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or a stereoisomer or tautomer thereof, or a pharmaceutically acceptable salt of any of the foregoing, m is 1; n is an integer from 0 to 4; X is —O—; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆ alkyl, C₃₋₁₀cycloalkyl or C₁₋₆haloalkyl, wherein the C₁₋₆ alkyl of R⁵ and R⁷ are each independently optionally substituted with one or more halo, or CN; R⁶ and R⁹ are each independently H, or halo; R⁸ is —C(O)N(R⁴)₂, C₃₋₁₀-cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11b}a or R^{11b} to form a C₃₋₁₀cycloalkyl; each R^{a} is, independently at each occurrence, —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, 4-10 membered heterocyclyl, or C₃₋₁₀cycloalkyl, wherein the C₁₋₆alkyl, C₁₋₆alkoxy, 4-10 membered heterocyclyl or C₃₋₁₀cycloalkyl of R^{a} is optionally substituted with one or more R^{b}; and each R^{b} is, independently at each occurrence, —OH, halo, C₁₋₆ alkyl or C₁₋₆ alkoxy. In some embodiments, m is an integer from 1 to 2; n is 1; X is —O—; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; each R^{a} is, independently at each occurrence, —OH, oxo, —CN, halo, C₁₋₃alkyl, C₁₋₃alkoxy, C₁₋₆haloalkyl, —O—C₁₋₃haloalkyl, —NH₂, —NH(C₁₋₃alkyl), —N(C₁₋₃alkyl)₂, 4-6 membered heterocyclyl, or C₃₋₆cycloalkyl, wherein the C₁₋₃alkyl, C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy.

In some embodiments of a compound of formula (I), or a stereoisomer or tautomer thereof, or a pharmaceutically acceptable salt of any of the foregoing, m is 1; n is an integer from 0 to 4; X is —O—; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀-cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11b}a or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is 1; X is —O—; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, m is 1; n is an integer from 0 to 4; X is —S—; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀-cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11b}a or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is 1; X is —S—; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, m is 1; n is an integer from 0 to 4; X is —C(R^{12a})(R^{12b})—; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀-cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11b}a and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11b}a or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is 1; X is —C(R^{12a})(R^{12b})—; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, this embodiment also applies to any other applicable formula detailed herein, such as a compound of formula (II), (I′), (I-A), (I-A1), (I-A2), (I-A3), (I-A4), (I-A5), (I-A6), (I-B), (I-B31), (I-B32), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, m is 1; n is an integer from 0 to 4; X is —N(H)—, or —N(C₁₋₆alkyl)-; R¹ is —NH—; R² is H; R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-10 membered heterocyclyl optionally substituted with one or more R^{a}; R⁵ and R⁷ are each independently H, halo, C₁₋₆alkyl, C₃₋₁₀ cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11a} or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is 1; X is —N(H)—, or —N(C₁₋₆alkyl)-; R¹ is —NH—; R² is H; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₆alkyl, C₁₋₆haloalkyl, or C₃₋₁₀cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11a} and R^{11b} are each independently at each occurrence, H; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, m is an integer from 1 to 4; n is an integer from 0 to 4; X is —O—, —S—, —C(R^{12a})(R^{12b})—, —N(H)—, or —N(C₁₋₆alkyl)-; R¹ is —NH—; R² is H; R³ is C₃₋₁₀cycloalkyl optionally substituted with one or more R^{a}; R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN; R⁶ and R⁹ are each independently H, or halo; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a}; R¹⁰ is H; R^{11a} and R^{11b} are each independently at each occurrence, H, or C₁₋₆alkyl, or taken together with one of R^{12a} or R^{12b} to form a C₃₋₁₀cycloalkyl; R^{12a} and R^{12b} are each independently, H, or C₁₋₆alkyl, or one of R^{12a} and R^{12b} is H, or C₁₋₆alkyl, and the other of R^{12a} and R^{12b} is taken together with one of R^{11a} or R^{11b} to form a C₃₋₁₀cycloalkyl; and each R^{a} is, independently at each occurrence, —OH, halo, C₁₋₆alkyl, C₁₋₆haloalkyl, or C₃₋₁₀cycloalkyl. In some embodiments, m is an integer from 1 to 2; n is an integer from 0 to 2; X is —O—; R¹ is —NH—; R² is H; R³ is C₃₋₆cycloalkyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}; R¹⁰ is H; R^{11a} and R^{11b} are each independently at each occurrence, H, or C₁₋₃alkyl, or taken together with one of R^{12a} or R^{12b} to form a C₃₋₆cycloalkyl; R^{12a} and R^{12b} are each independently, H, or C₁₋₃alkyl, or one of R^{12a} and R^{12b} is H, or C₁₋₆alkyl, and the other of R^{12a} and R^{12b} is taken together with one of R^{11a} or R^{11b} to form a C₃₋₆cycloalkyl; and each R^{a} is, independently at each occurrence, —OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by,

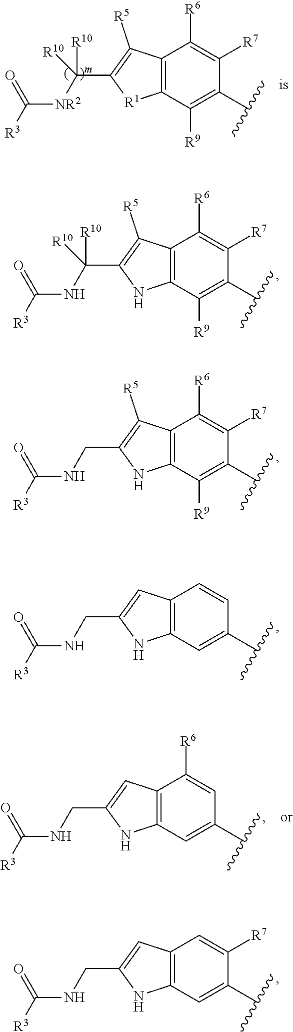

wherein R³ is C₁₋₆alkyl. In some embodiments, R³ is C₁₋₆alkyl; R⁵ and R⁷ are each independently H, halo, or C₁₋₆alkyl; and R⁶ and R⁹ are each independently H or halo. In some embodiments, the moiety represented by

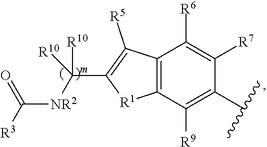

is selected from the group consisting of

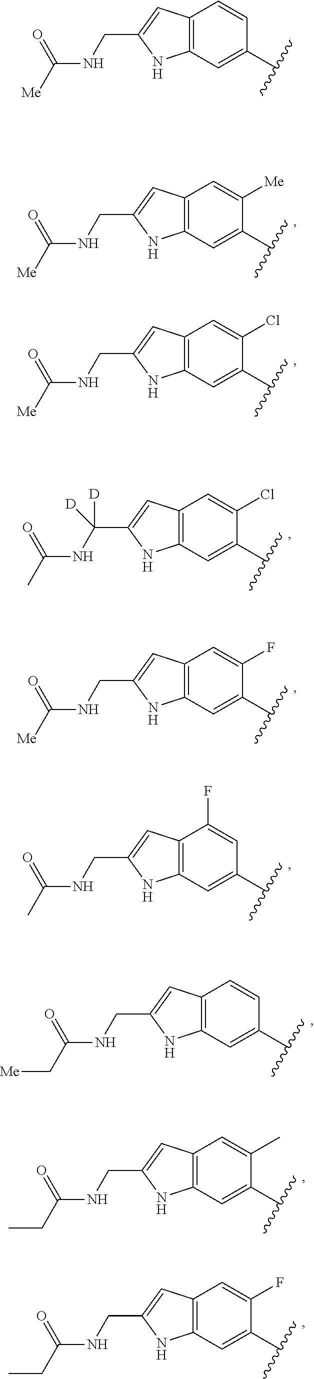

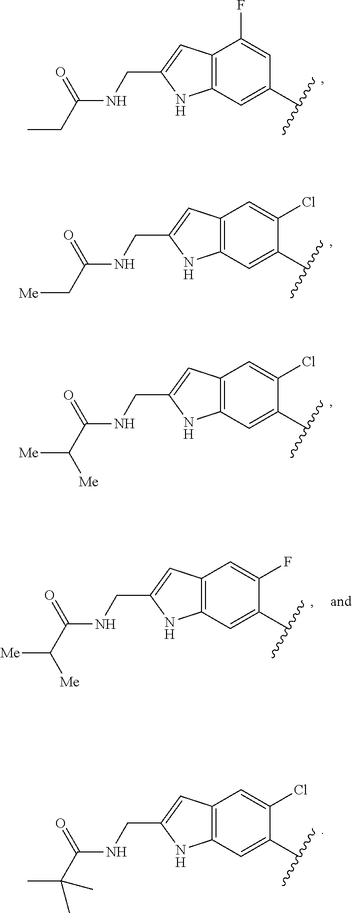 [SMILES: *c1cc(F)c2cc(CNC(=O)CC)[nH]c2c1 ; *c1cc2[nH]c(CNC(=O)CC)cc2cc1Cl ; *c1cc2[nH]c(CNC(=O)C(C)C)cc2cc1Cl ; *c1cc2[nH]c(CNC(=O)C(C)C)cc2cc1F ; *c1cc2[nH]c(CNC(=O)C(C)(C)C)cc2cc1Cl]

In some embodiments of a compound of formula (I), or a stereoisomer or tautomer thereof, or a pharmaceutically acceptable salt of any of the foregoing, the moiety represented by

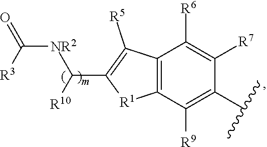

is selected from the group consisting of

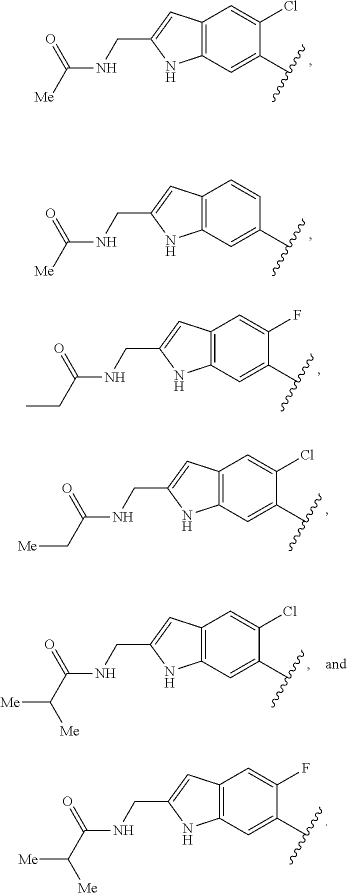 [SMILES: *c1cc2[nH]c(CNC(C)=O)cc2cc1Cl ; *c1ccc2cc(CNC(C)=O)[nH]c2c1 ; *c1cc2[nH]c(CNC(=O)CC)cc2cc1F ; *c1cc2[nH]c(CNC(=O)CC)cc2cc1Cl ; *c1cc2[nH]c(CNC(=O)C(C)C)cc2cc1Cl ; *c1cc2[nH]c(CNC(=O)C(C)C)cc2cc1F]

In some embodiments, the moiety represented by

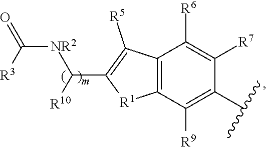

is selected from the group consisting of

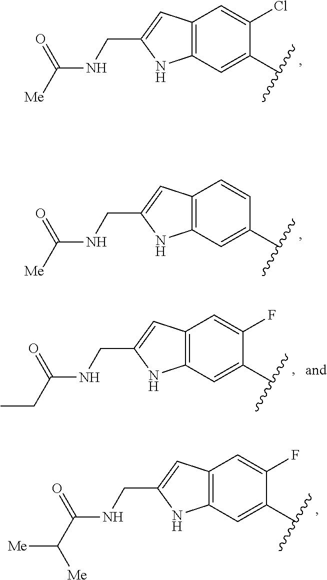 [SMILES: *c1cc2[nH]c(CNC(C)=O)cc2cc1Cl ; *c1ccc2cc(CNC(C)=O)[nH]c2c1 ; *c1cc2[nH]c(CNC(=O)CC)cc2cc1Cl ; *c1cc2[nH]c(CNC(=O)C(C)C)cc2cc1F]

In some embodiments, the moiety represented by

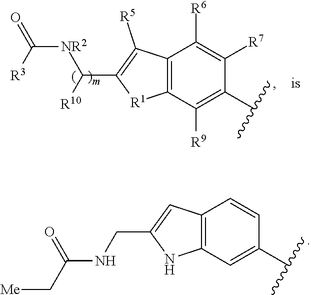

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, the moiety represented by

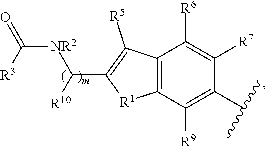

is selected from the group consisting of

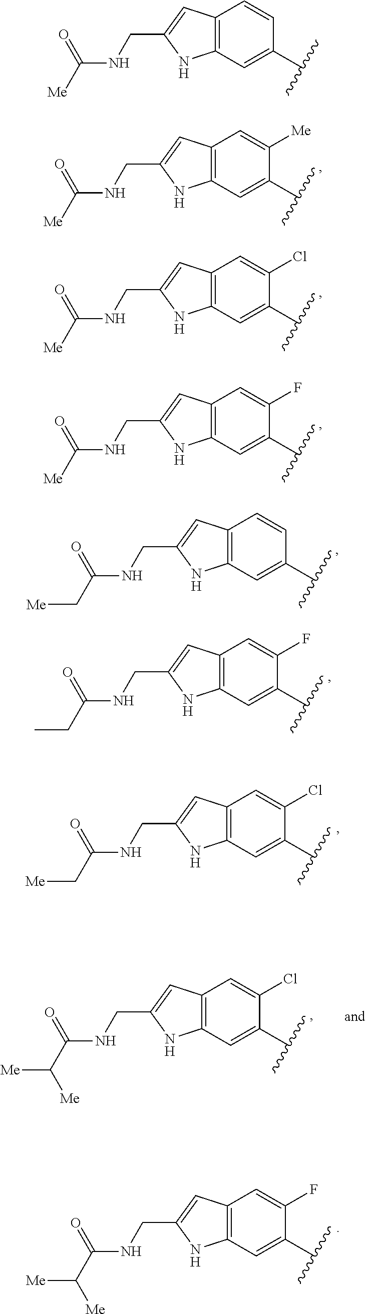

In some embodiments, the moiety represented by

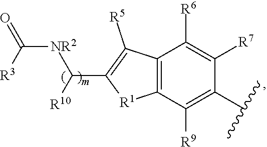

is selected from the group consisting of

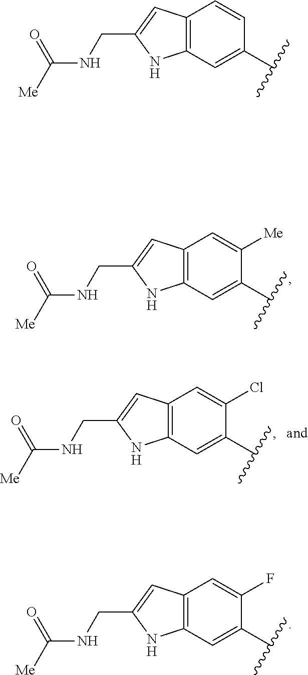 [SMILES: *c1ccc2cc(CNC(C)=O)[nH]c2c1 ; *c1cc2[nH]c(CNC(C)=O)cc2cc1C ; *c1cc2[nH]c(CNC(C)=O)cc2cc1Cl ; *c1cc2[nH]c(CNC(C)=O)cc2cc1F]

In some embodiments, the moiety represented by

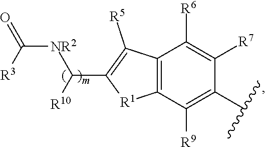

is

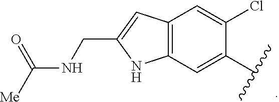 [SMILES: *c1cc2[nH]c(CNC(C)=O)cc2cc1Cl]

In some embodiments, the moiety represented by

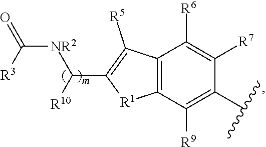

is

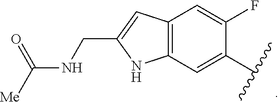 [SMILES: *c1cc2[nH]c(CNC(C)=O)cc2cc1F]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

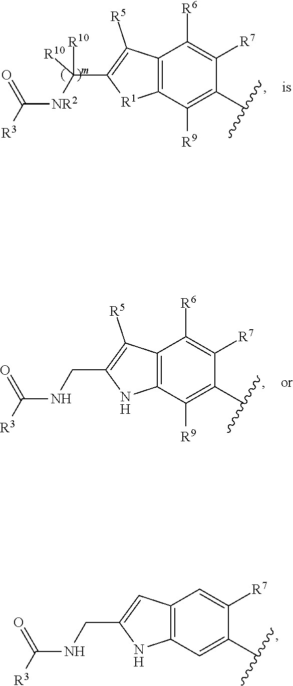

wherein R³ is C₁₋₆alkyl optionally substituted with one or more R^{a}. In some embodiments, R³ is C₁₋₆alkyl substituted with one or more R^{a}; R⁵ and R⁷ are each H or halo; and R⁶ and R⁹ are each H. In some embodiments, the moiety represented by

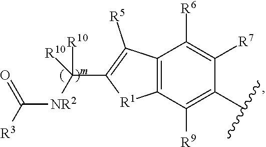

is selected from the group consisting of

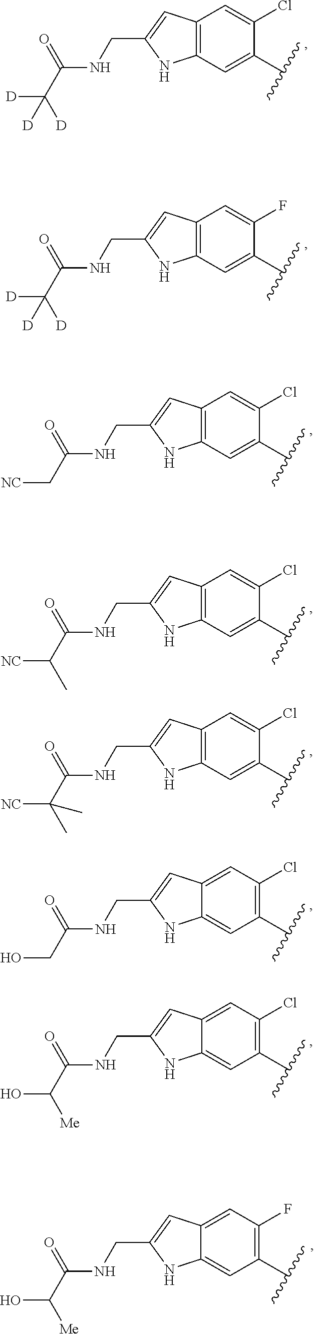

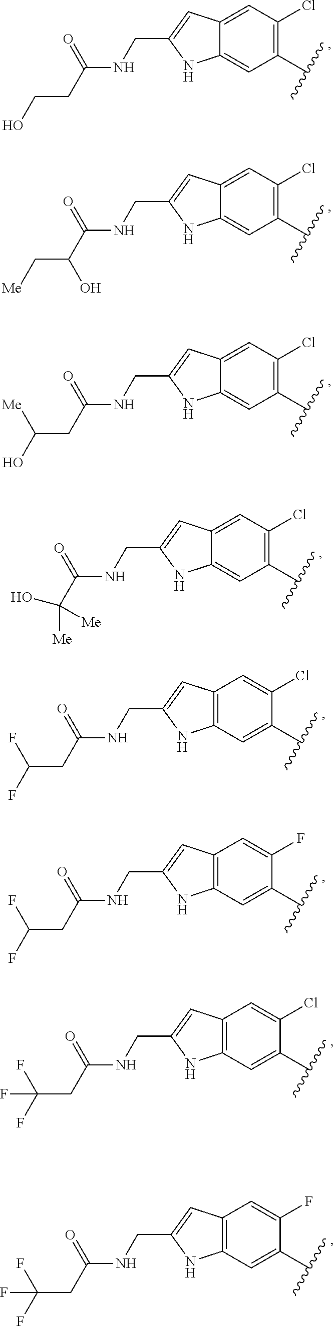

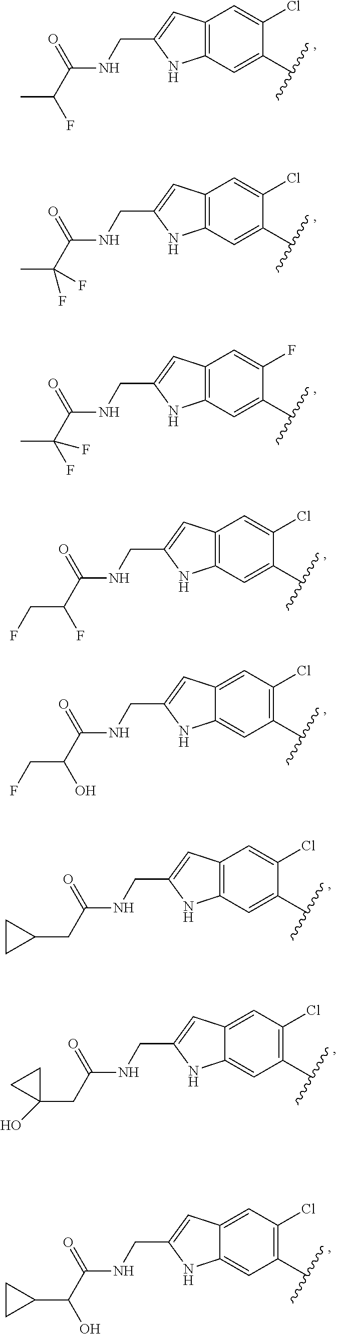

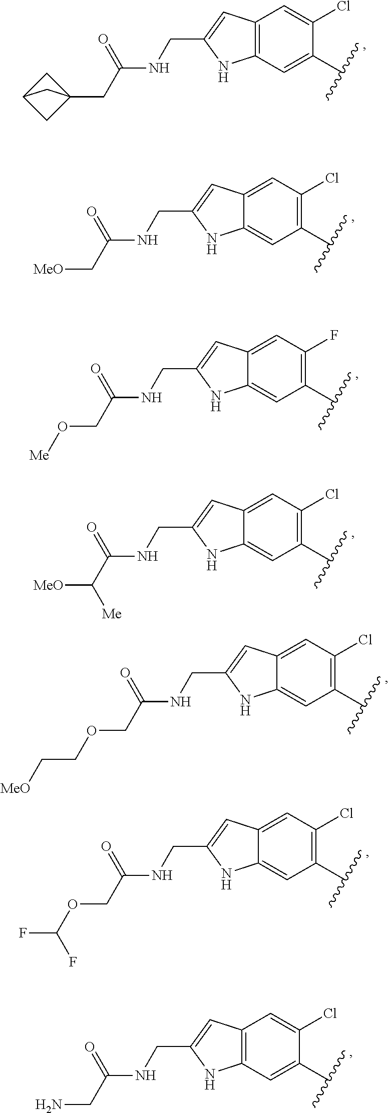

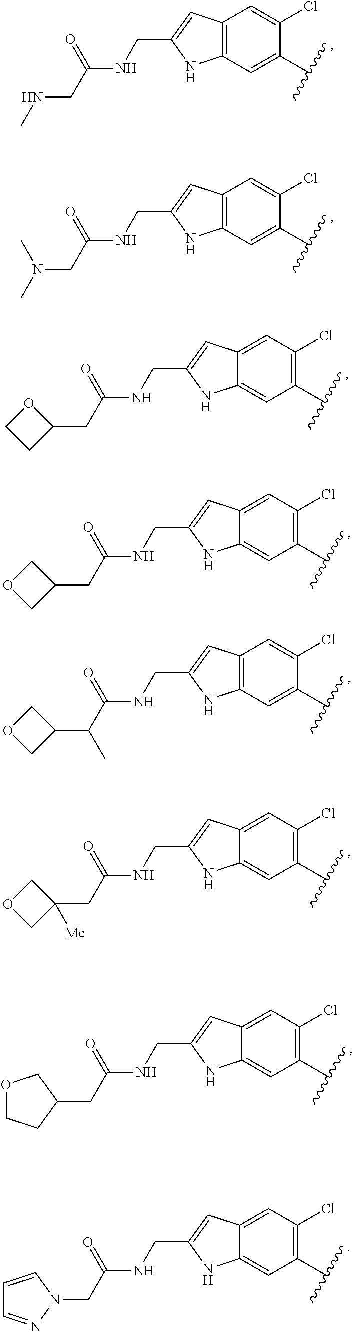

In some embodiments of a compound of formula (I′), or a stereoisomer or tautomer thereof, or a pharmaceutically acceptable salt of any of the foregoing, the moiety represented by

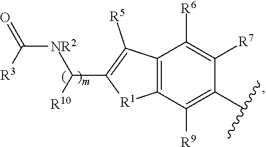

is selected from the group consisting of

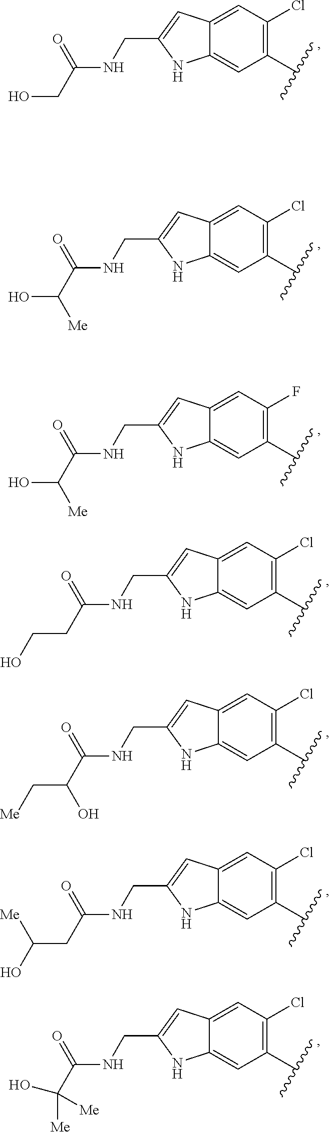

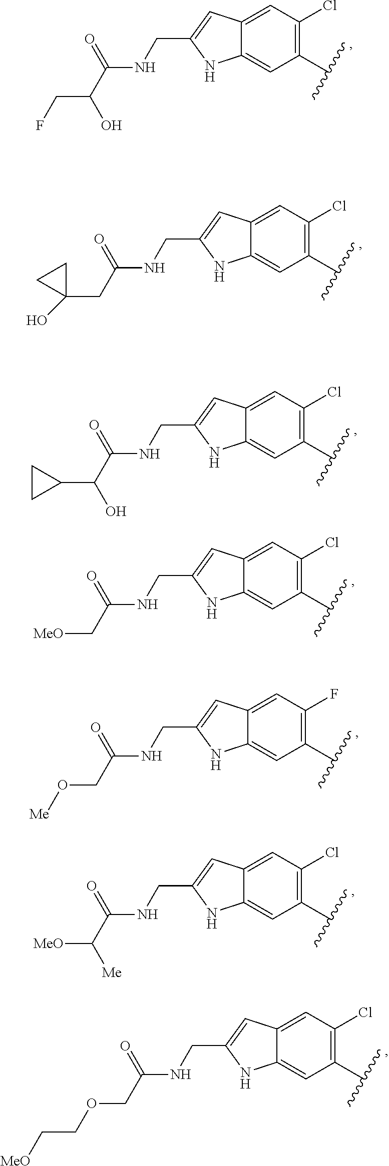

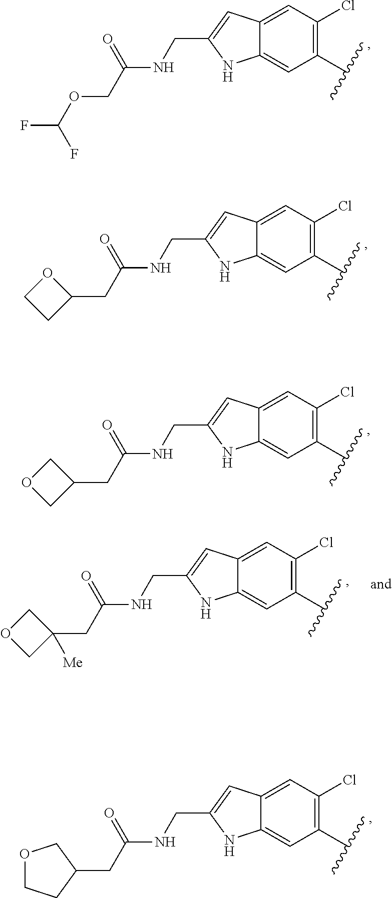 [SMILES: *c1cc2[nH]c(CNC(=O)COC(F)F)cc2cc1Cl ; *c1cc2[nH]c(CNC(=O)CC3CCO3)cc2cc1Cl ; *c1cc2[nH]c(CNC(=O)CC3COC3)cc2cc1Cl ; *c1cc2[nH]c(CNC(=O)CC3(C)COC3)cc2cc1Cl ; *c1cc2[nH]c(CNC(=O)CC3CCOC3)cc2cc1Cl]

In some embodiments, the moiety represented by

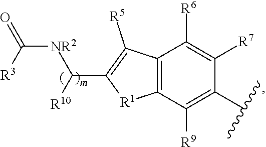

is selected from the group consisting of

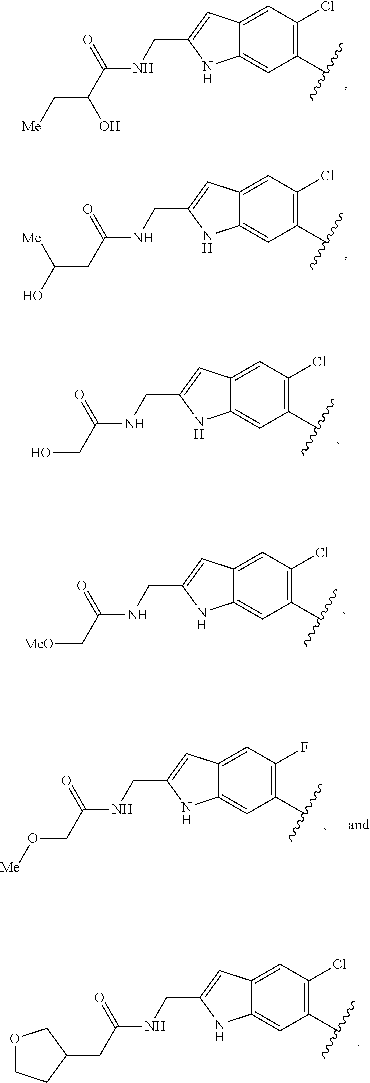 [SMILES: *c1cc2[nH]c(CNC(=O)C(O)CC)cc2cc1Cl ; *c1cc2[nH]c(CNC(=O)CC(C)O)cc2cc1Cl ; *c1cc2[nH]c(CNC(=O)CO)cc2cc1Cl ; *c1cc2[nH]c(CNC(=O)COC)cc2cc1Cl ; *c1cc2[nH]c(CNC(=O)COC)cc2cc1F ; *c1cc2[nH]c(CNC(=O)CC3CCOC3)cc2cc1Cl]

In some embodiments of a compound of formula (II), or a stereoisomer or tautomer thereof, or a pharmaceutically acceptable salt of any of the foregoing, the moiety represented by

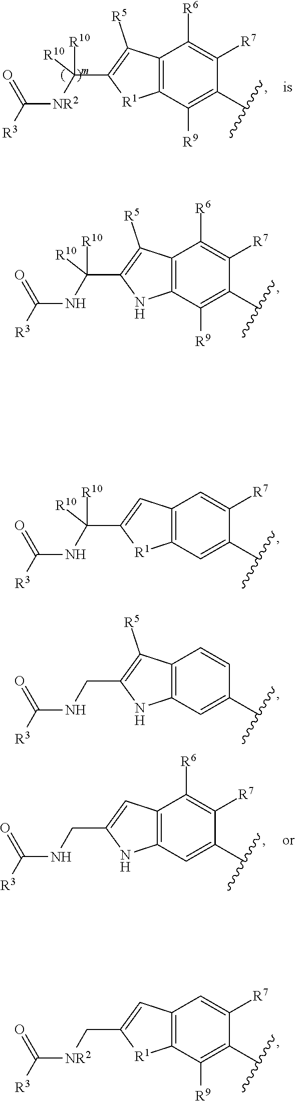

wherein R³ is C₃₋₆cycloalkyl optionally substituted with one or more R^{a}. In some embodiments, R³ is C₃₋₆cycloalkyl substituted with one or more R^{a}; R⁵ and R⁷ are each independently H, halo, C₁₋₆alkyl, C₁₋₆alkoxy, C₁₋₆ thioalkyl, C₃₋₁₀cycloalkyl, C₁₋₆ haloalkyl, or 5-20 membered heteroaryl, wherein the C₁₋₆alkyl, C₁₋₆alkoxy, or C₃₋₁₀cycloalkyl of R⁵ and R⁷ are each independently optionally substituted with one or more halo or CN; and R⁶ and R⁹ are each independently H or halo. In some embodiments, the moiety represented by

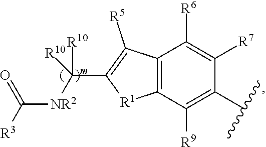

is selected from the group consisting of

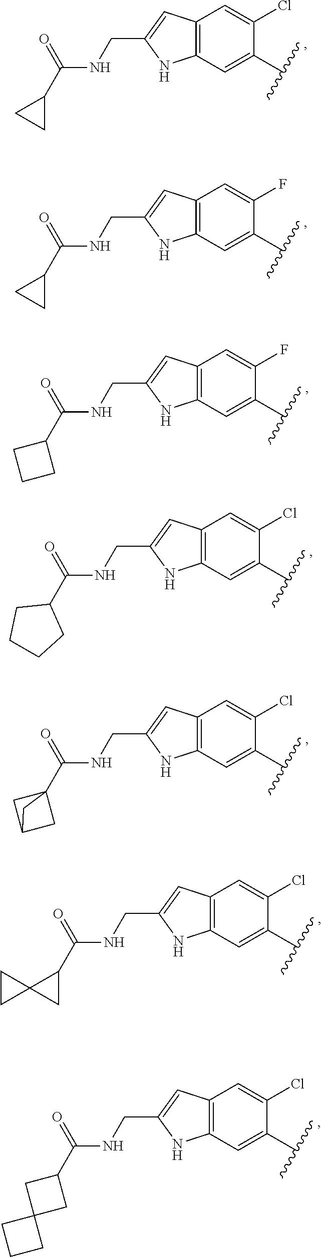

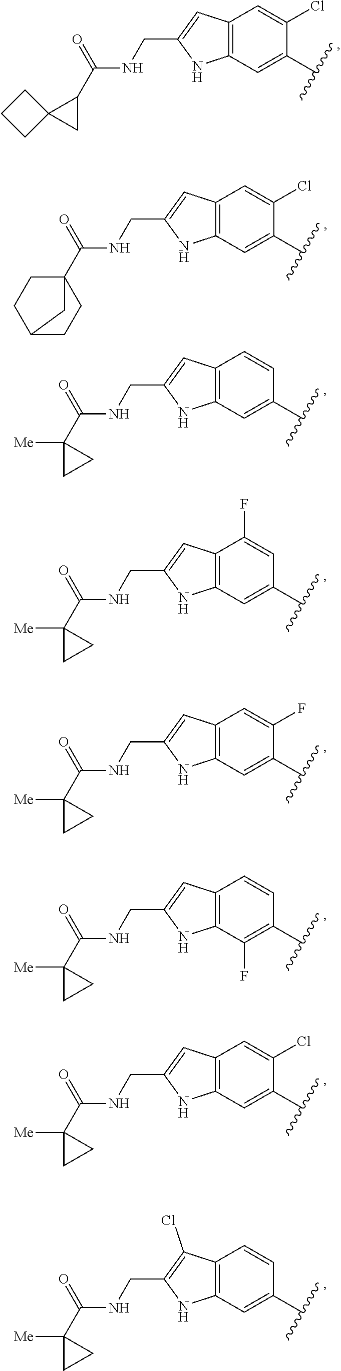

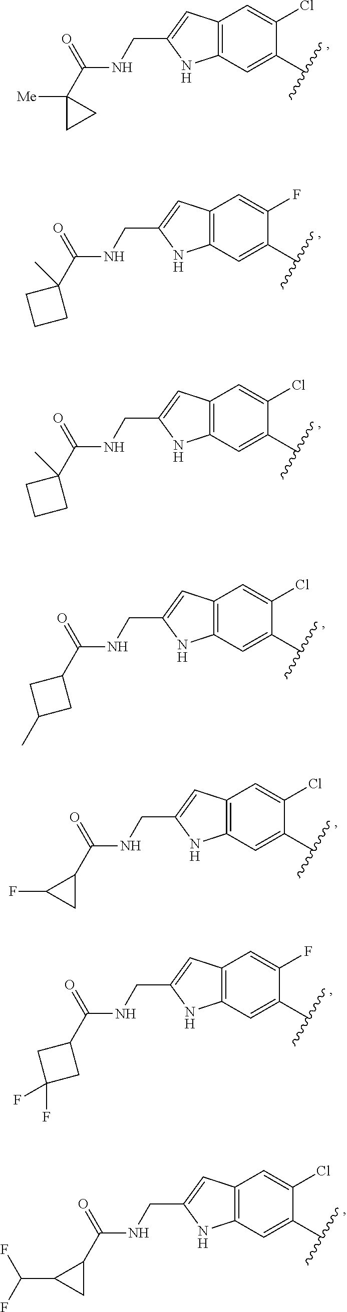

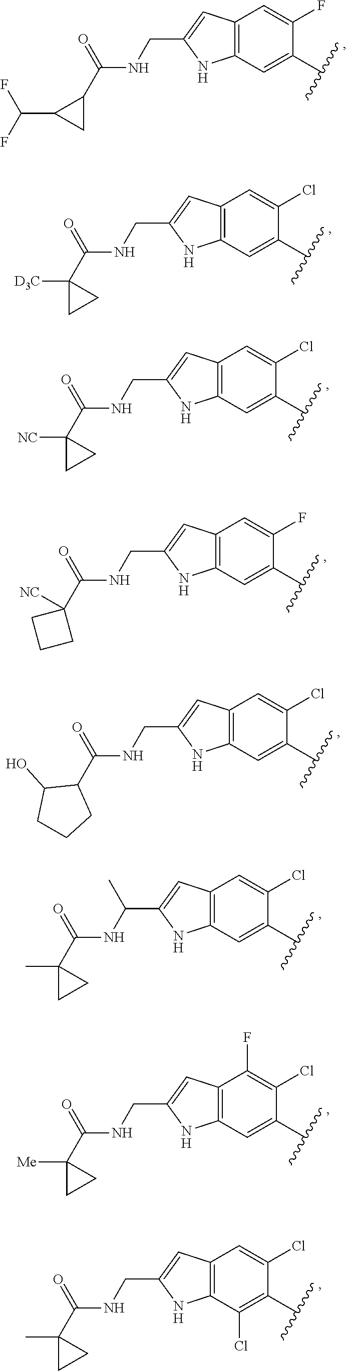

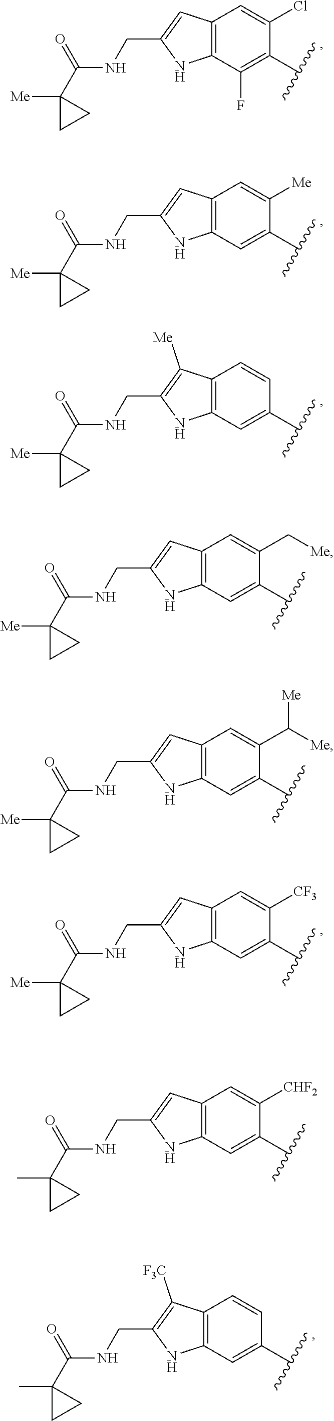

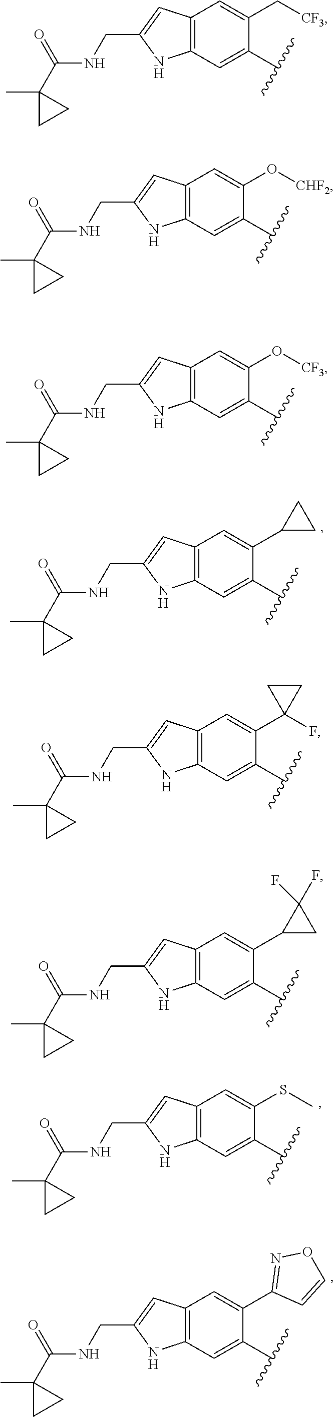

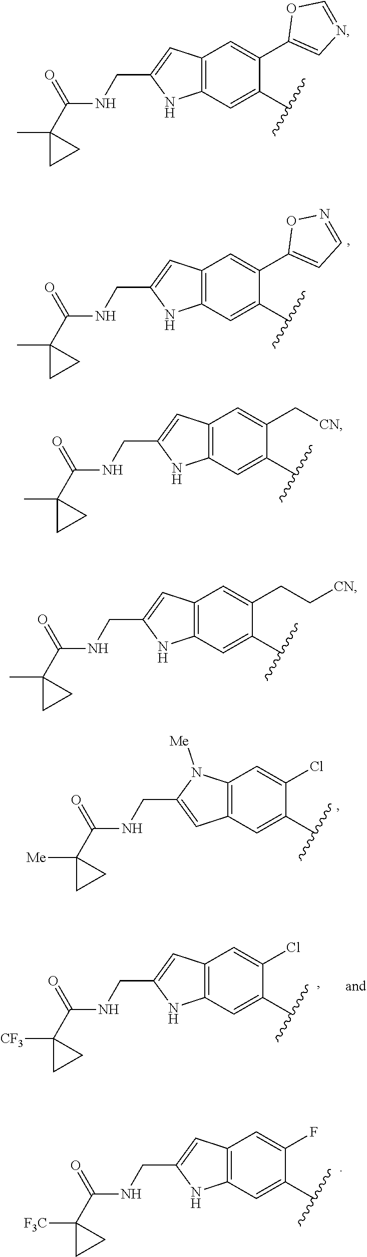

In some embodiments of a compound of formula (I), or a stereoisomer or tautomer thereof, or a pharmaceutically acceptable salt of any of the foregoing, the moiety represented by

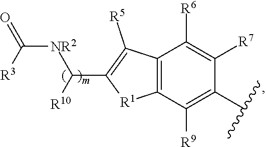

is selected from the group consisting of

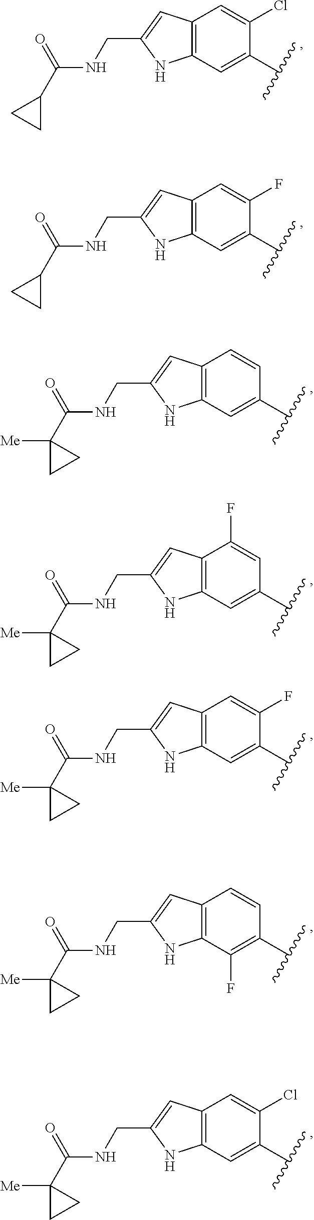

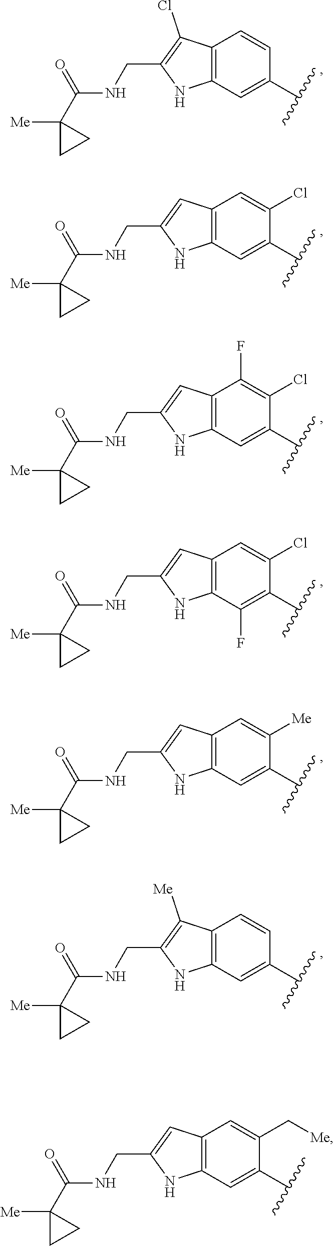

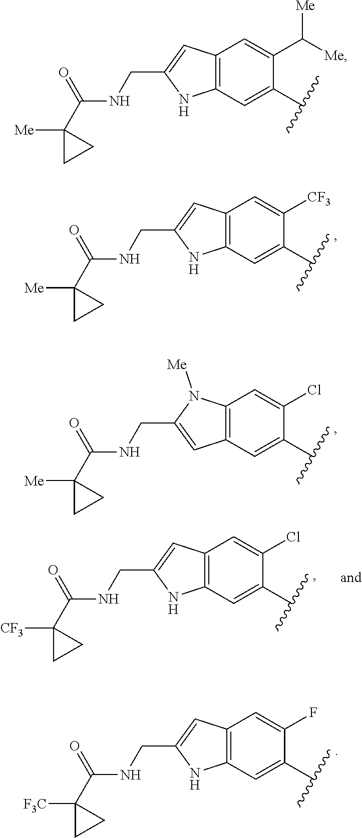 [SMILES: *c1cc2[nH]c(CNC(=O)C3(C)CC3)cc2cc1C(C)C ; *c1cc2[nH]c(CNC(=O)C3(C)CC3)cc2cc1C(F)(F)F ; *c1cc2cc(CNC(=O)C3(C)CC3)n(C)c2cc1Cl ; *c1cc2[nH]c(CNC(=O)C3(C(F)(F)F)CC3)cc2cc1Cl ; *c1cc2[nH]c(CNC(=O)C3(C(F)(F)F)CC3)cc2cc1F]

In some embodiments, the moiety represented by

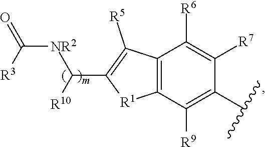

is selected from the group consisting of

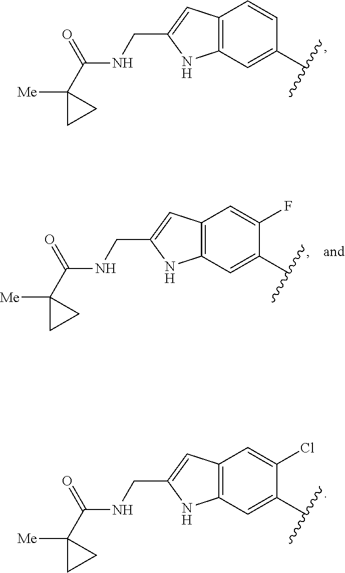 [SMILES: *c1ccc2cc(CNC(=O)C3(C)CC3)[nH]c2c1 ; *c1cc2[nH]c(CNC(=O)C3(C)CC3)cc2cc1F ; *c1cc2[nH]c(CNC(=O)C3(C)CC3)cc2cc1Cl]

In some embodiments, the moiety represented by

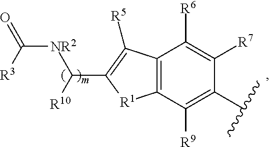

is

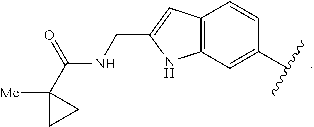 [SMILES: *c1ccc2cc(CNC(=O)C3(C)CC3)[nH]c2c1]

In some embodiments, the moiety represented by

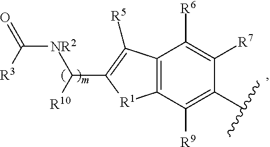

is

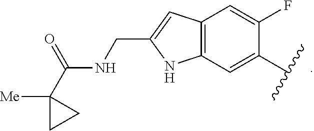 [SMILES: *c1cc2[nH]c(CNC(=O)C3(C)CC3)cc2cc1F]

In some embodiments, the moiety represented by

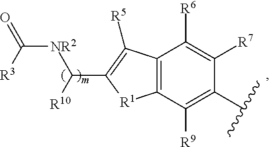

is

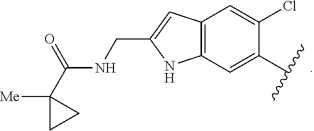 [SMILES: *c1cc2[nH]c(CNC(=O)C3(C)CC3)cc2cc1Cl]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, the moiety represented by

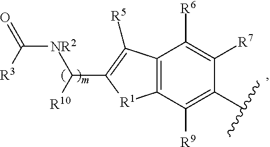

is selected from the group consisting of

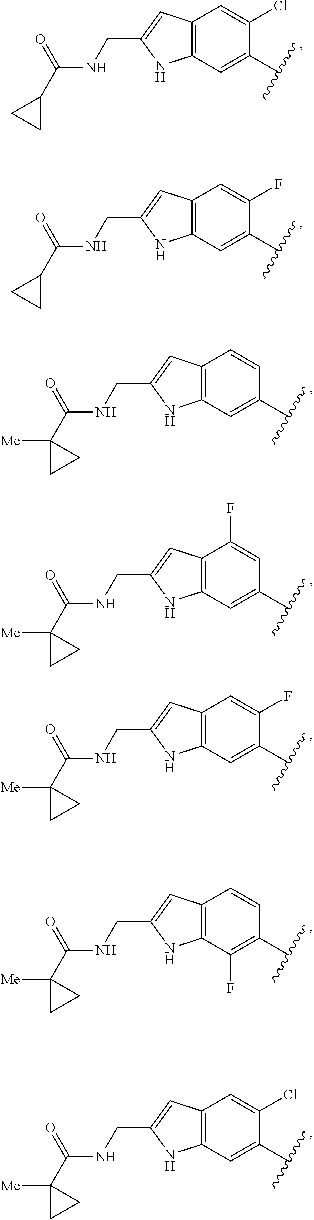

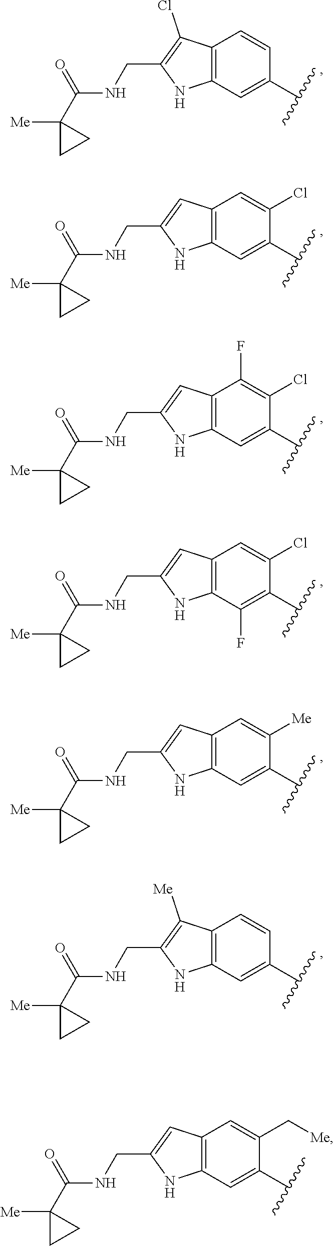

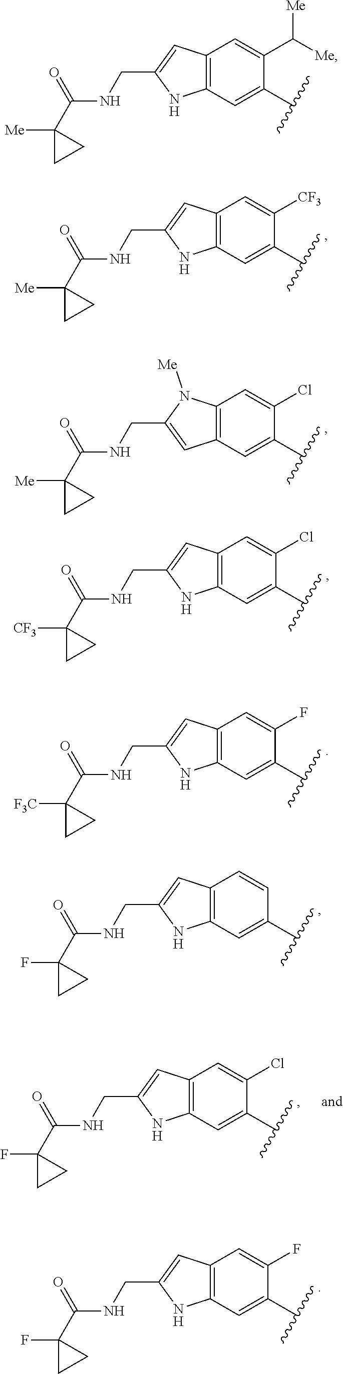

In some embodiments, the moiety represented by

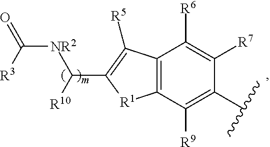

is selected from the group consisting of

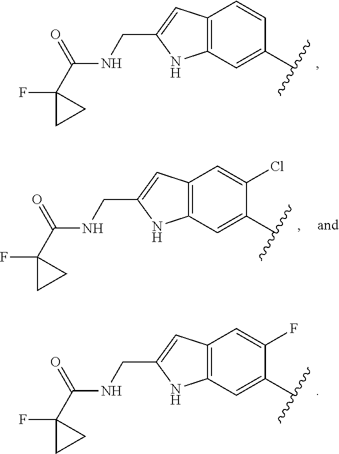 [SMILES: *c1ccc2cc(CNC(=O)C3(F)CC3)[nH]c2c1 ; *c1cc2[nH]c(CNC(=O)C3(F)CC3)cc2cc1Cl ; *c1cc2[nH]c(CNC(=O)C3(F)CC3)cc2cc1F]

In some embodiments, the moiety represented by

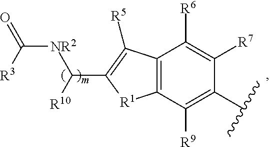

is

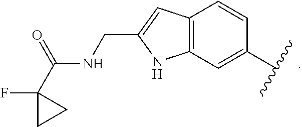 [SMILES: *c1ccc2cc(CNC(=O)C3(F)CC3)[nH]c2c1]

In some embodiments, the moiety represented by

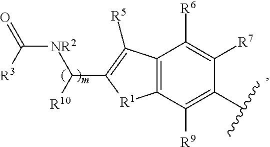

is

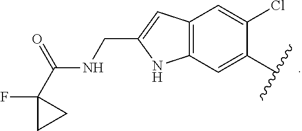 [SMILES: *c1cc2[nH]c(CNC(=O)C3(F)CC3)cc2cc1Cl]

In some embodiments, the moiety represented by

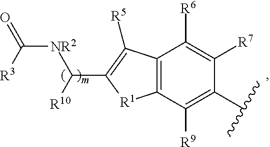

is

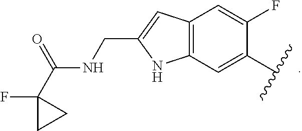 [SMILES: *c1cc2[nH]c(CNC(=O)C3(F)CC3)cc2cc1F]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or a stereoisomer or tautomer thereof, or a pharmaceutically acceptable salt of any of the foregoing, the moiety represented by

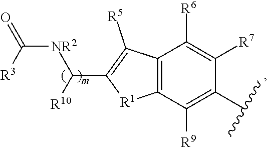

is selected from the group consisting of

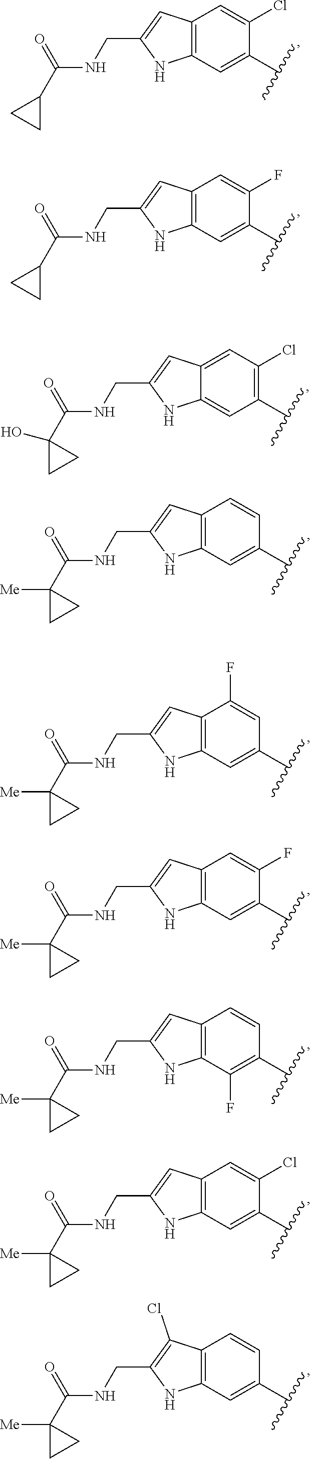

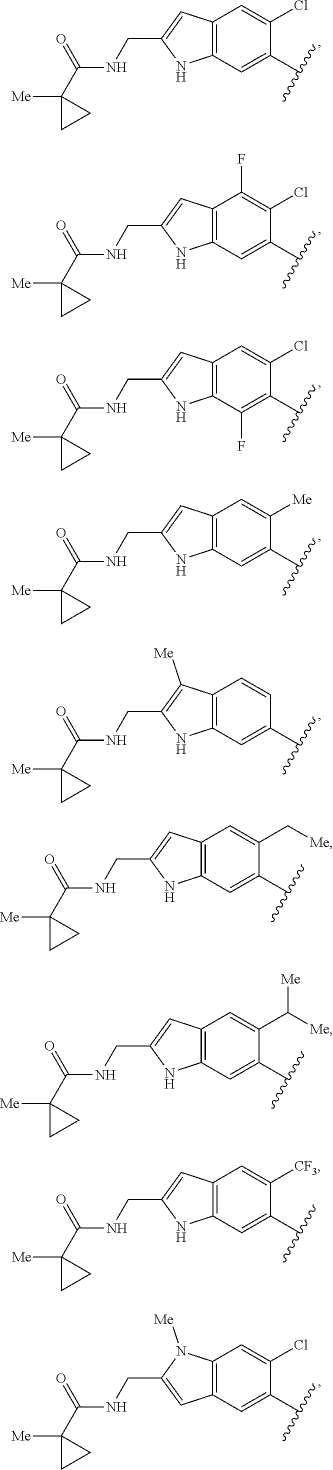

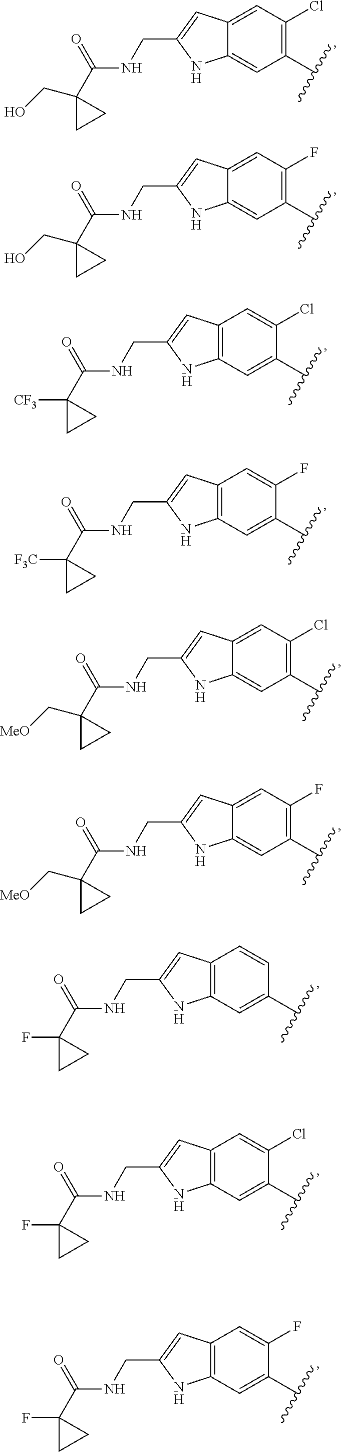

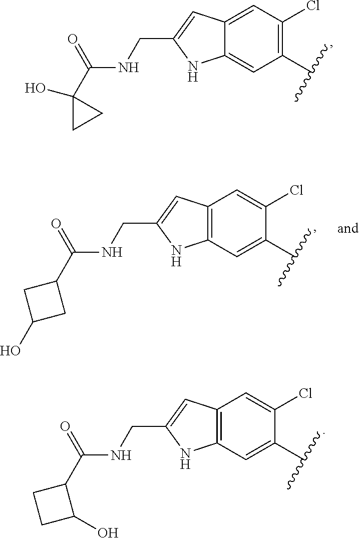 [SMILES: *c1cc2[nH]c(CNC(=O)C3(O)CC3)cc2cc1Cl ; *c1cc2[nH]c(CNC(=O)C3CC(O)C3)cc2cc1Cl ; *c1cc2[nH]c(CNC(=O)C3CCC3O)cc2cc1Cl]

In some embodiments of a compound of formula (II), or a stereoisomer or tautomer thereof, or a pharmaceutically acceptable salt of any of the foregoing, the moiety represented by

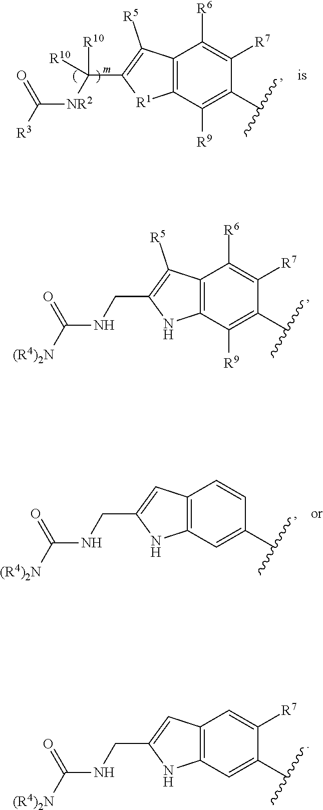

In some embodiments, R⁵ and R⁷ are each independently H, or halo; and R⁶ and R⁹ are each H. In some embodiments, the moiety represented by

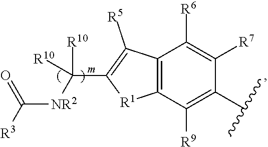

is selected from the group consisting of

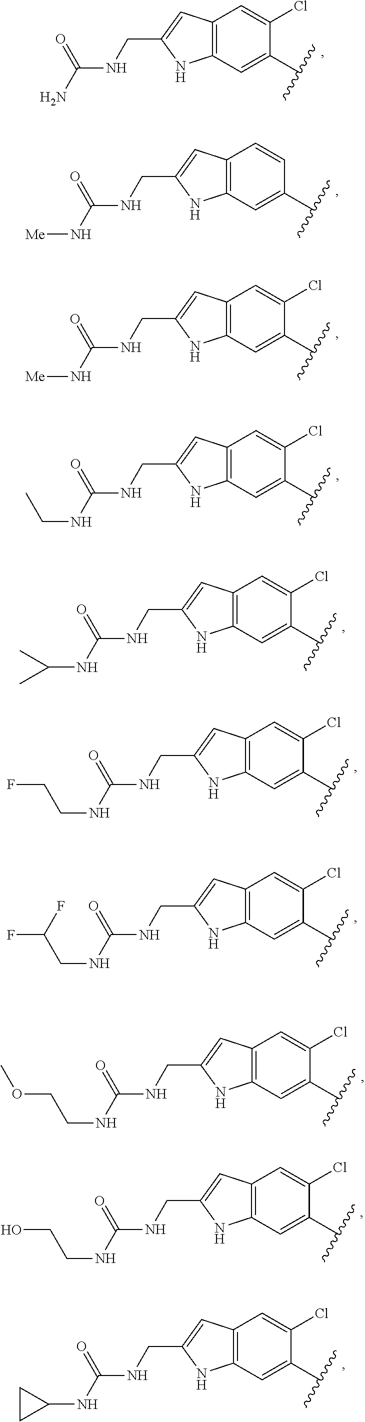

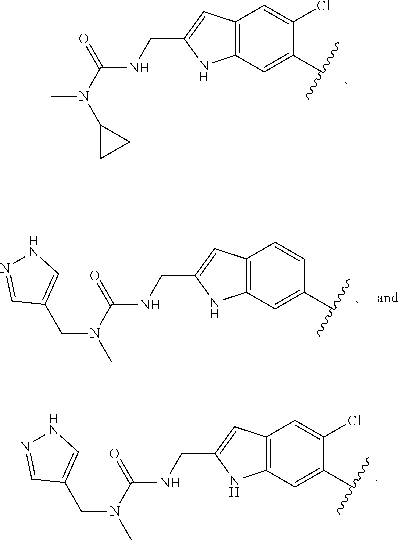 [SMILES: *c1cc2[nH]c(CNC(=O)N(C)C3CC3)cc2cc1Cl ; *c1ccc2cc(CNC(=O)N(C)Cc3cn[nH]c3)[nH]c2c1 ; *c1cc2[nH]c(CNC(=O)N(C)Cc3cn[nH]c3)cc2cc1Cl]

In some embodiments, R⁵ and R⁷ are each independently H, halo, C₁₋₆ alkyl, C₁₋₆alkoxy, or C₁₋₆haloalkyl, wherein the C₁₋₆ alkyl, C₁₋₆alkoxy, or C₃₋₁₀cycloalkyl of R⁵ and R⁷ are each independently optionally substituted with one or more halo; and R⁶ and R⁹ are each independently H or halo. In some embodiments, the moiety represented by

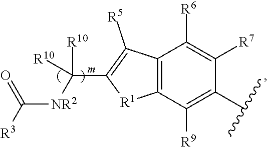

is selected from the group consisting of

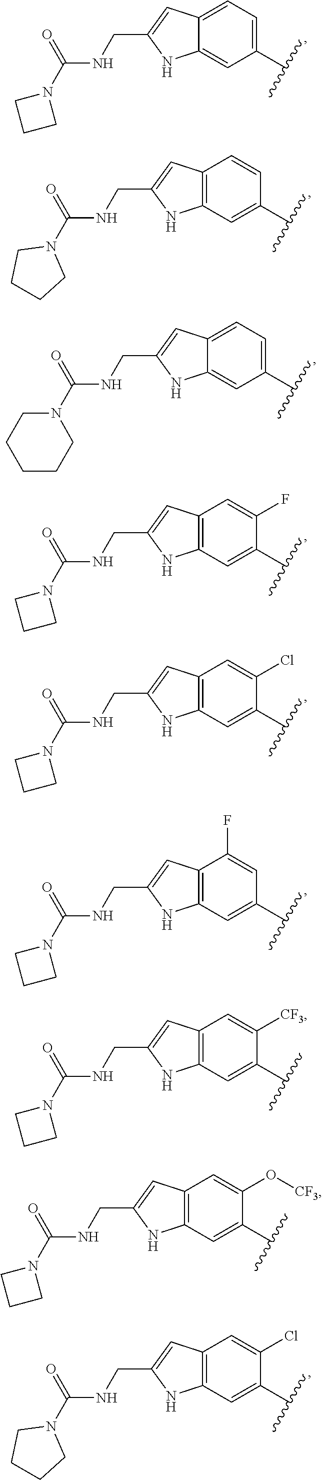

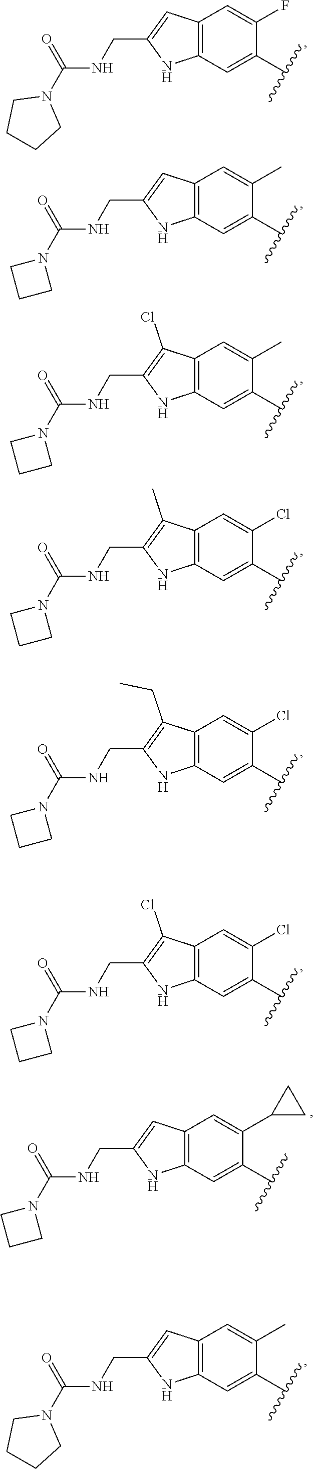

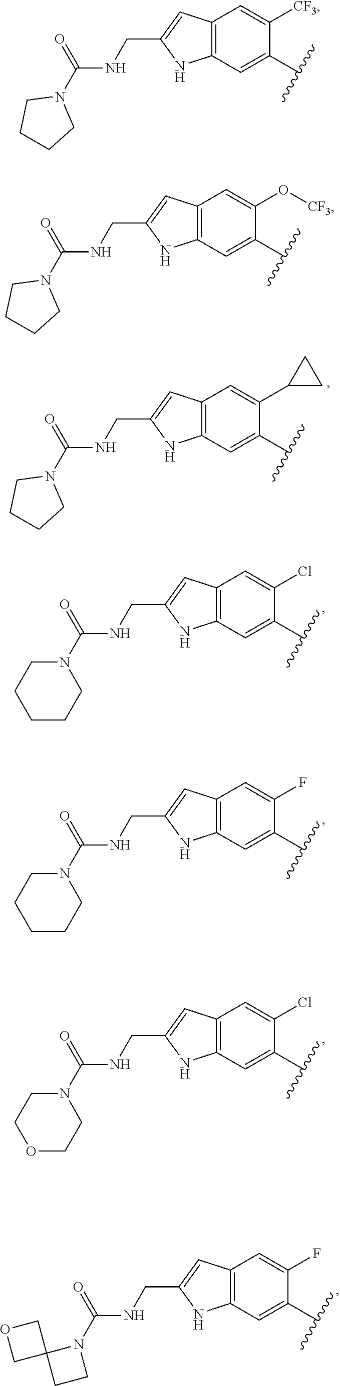

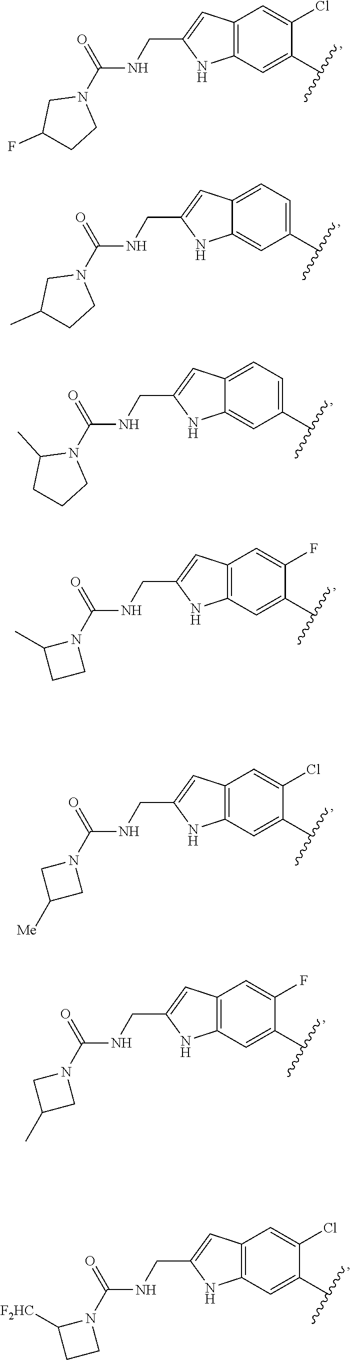

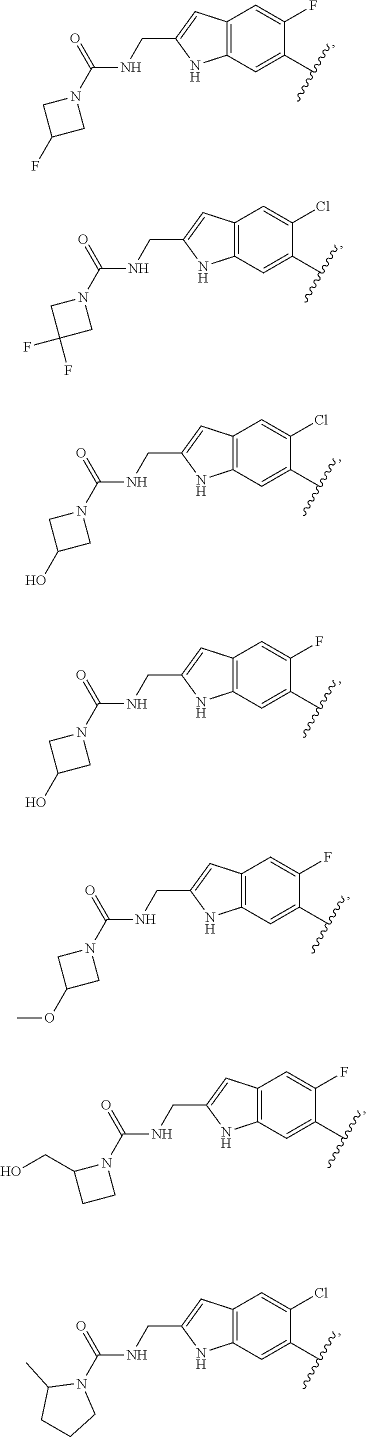

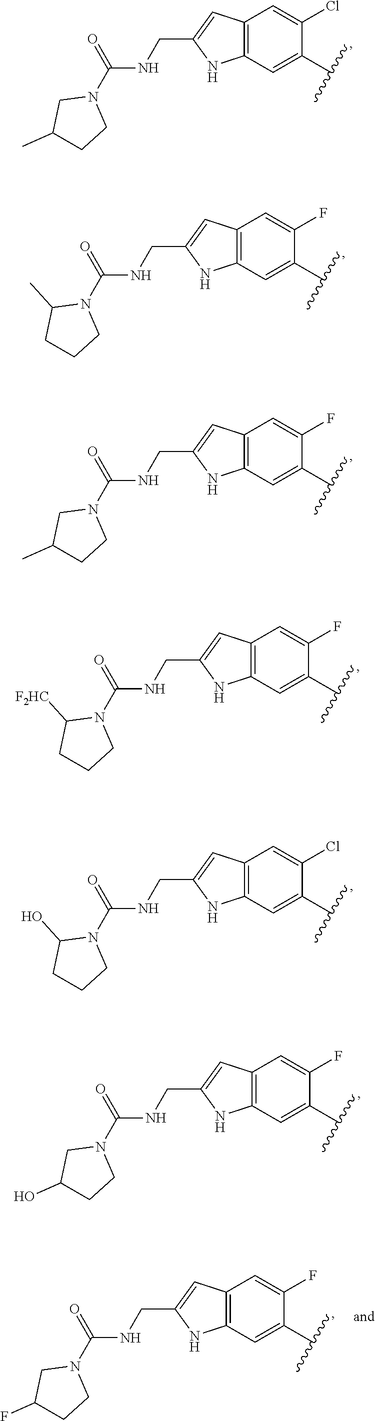

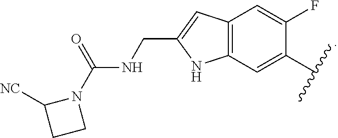 [SMILES: *c1cc2[nH]c(CNC(=O)N3CCC3C#N)cc2cc1F]

In some embodiments of a compound of formula (I), or a stereoisomer or tautomer thereof, or a pharmaceutically acceptable salt of any of the foregoing, the moiety represented by

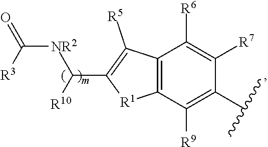

is selected from the group consisting of

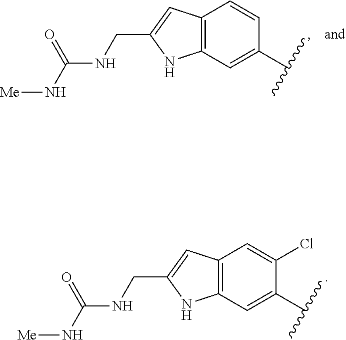 [SMILES: *c1ccc2cc(CNC(=O)NC)[nH]c2c1 ; *c1cc2[nH]c(CNC(=O)NC)cc2cc1Cl]

In some embodiments, the moiety represented by

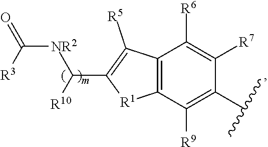

is selected from the group consisting of

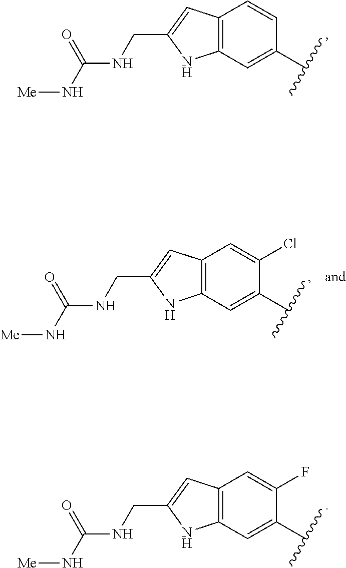 [SMILES: *c1ccc2cc(CNC(=O)NC)[nH]c2c1 ; *c1cc2[nH]c(CNC(=O)NC)cc2cc1Cl ; *c1cc2[nH]c(CNC(=O)NC)cc2cc1F]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, the moiety represented by

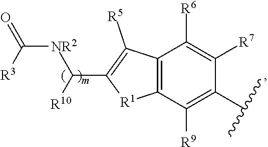

is selected from the group consisting of

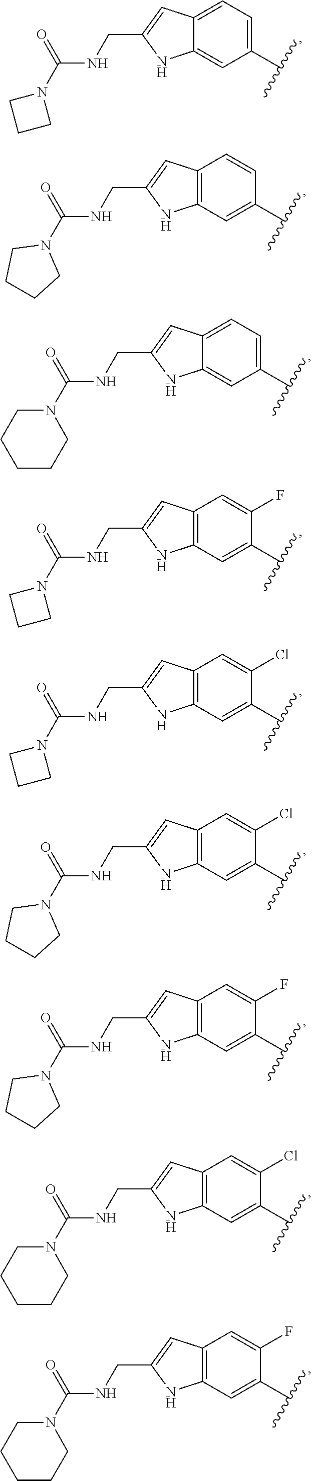

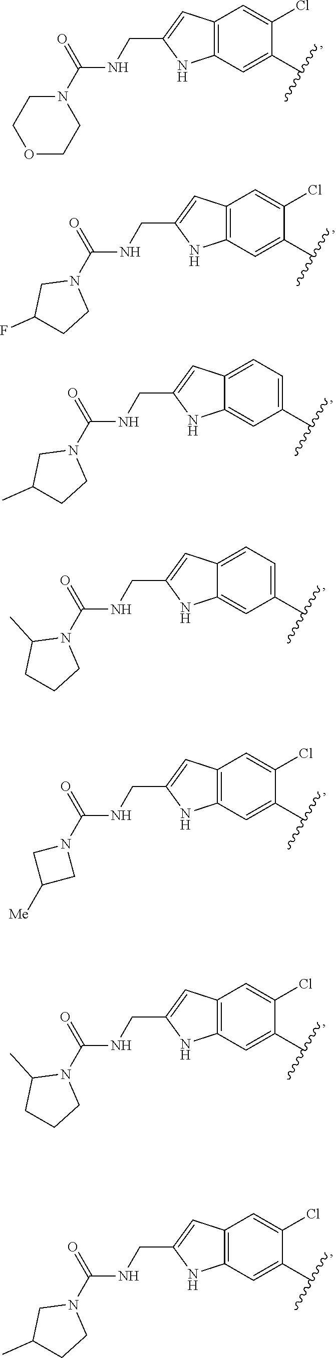

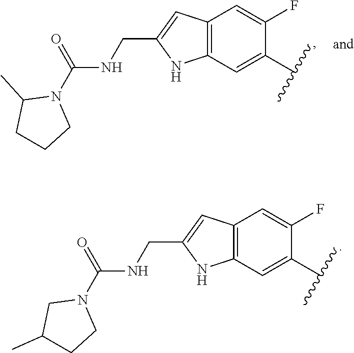 [SMILES: *c1cc2[nH]c(CNC(=O)N3CCCC3C)cc2cc1F ; *c1cc2[nH]c(CNC(=O)N3CCC(C)C3)cc2cc1F]

In some embodiments, the moiety represented by

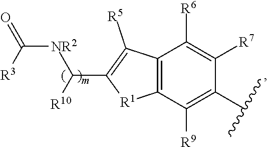

is selected from the group consisting of

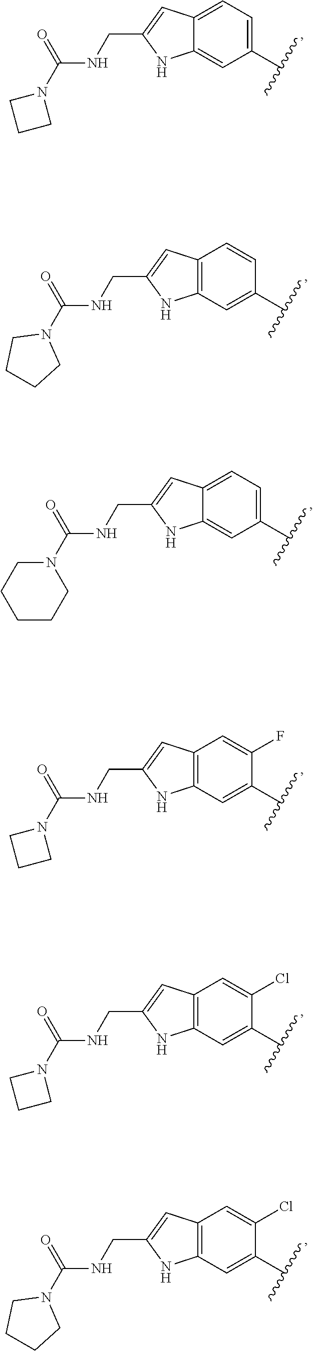 [SMILES: *c1ccc2cc(CNC(=O)N3CCC3)[nH]c2c1 ; *c1ccc2cc(CNC(=O)N3CCCC3)[nH]c2c1 ; *c1ccc2cc(CNC(=O)N3CCCCC3)[nH]c2c1 ; *c1cc2[nH]c(CNC(=O)N3CCC3)cc2cc1F ; *c1cc2[nH]c(CNC(=O)N3CCC3)cc2cc1Cl ; *c1cc2[nH]c(CNC(=O)N3CCCC3)cc2cc1Cl]

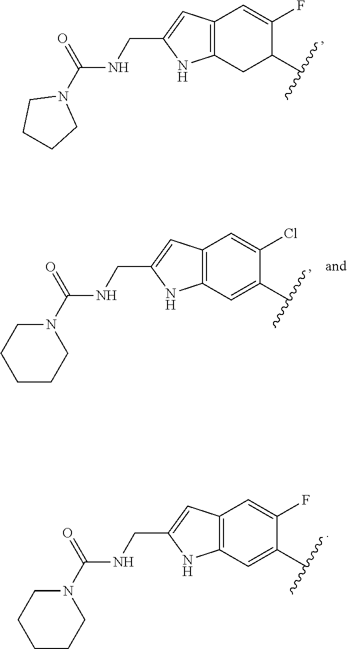 [SMILES: *C1Cc2[nH]c(CNC(=O)N3CCCC3)cc2C=C1F ; *c1cc2[nH]c(CNC(=O)N3CCCCC3)cc2cc1Cl ; *c1cc2[nH]c(CNC(=O)N3CCCCC3)cc2cc1F]

In some embodiments, the moiety represented by

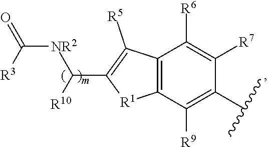

is selected from the group consisting of

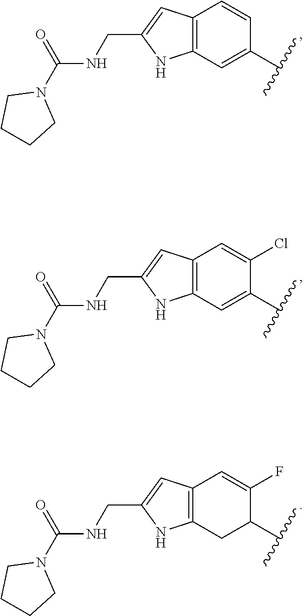 [SMILES: *c1ccc2cc(CNC(=O)N3CCCC3)[nH]c2c1 ; *c1cc2[nH]c(CNC(=O)N3CCCC3)cc2cc1Cl ; *C1Cc2[nH]c(CNC(=O)N3CCCC3)cc2C=C1F]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

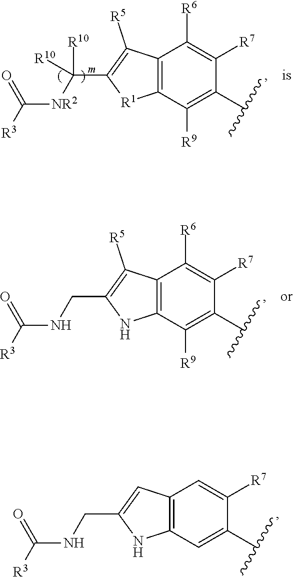

wherein R³ is 4-10 membered heterocyclyl optionally substituted with one or more R^{a}. In some embodiments, R³ is 4-10 membered heterocyclyl optionally substituted with one or more R^{a}; R⁵ and R⁷ are each independently halo; and R⁶ and R⁹ are each H. In some embodiments, the moiety represented by

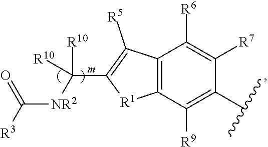

is selected from the group consisting of

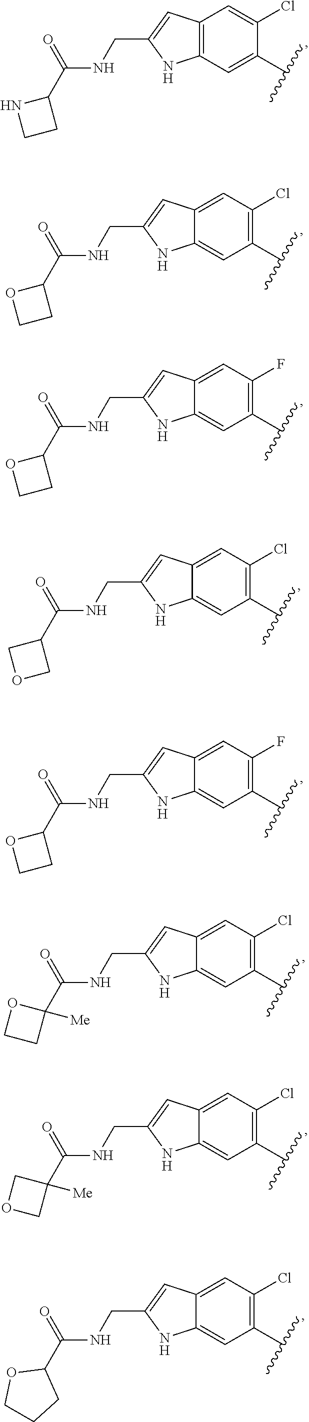

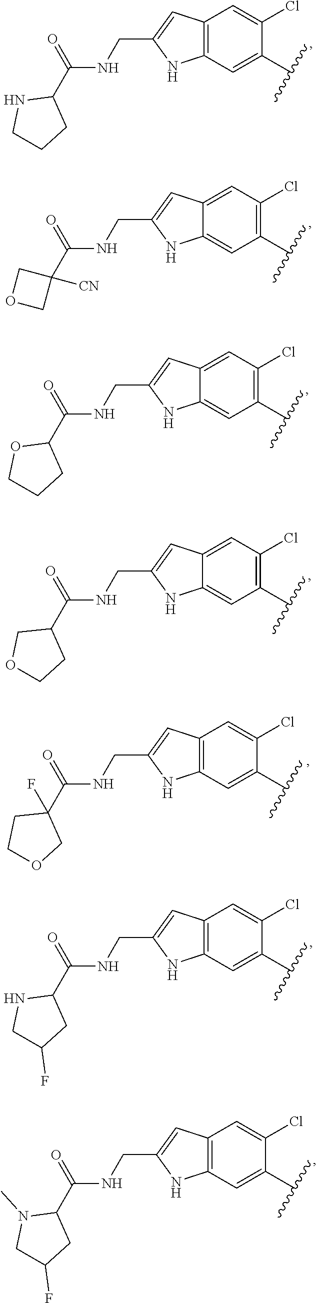

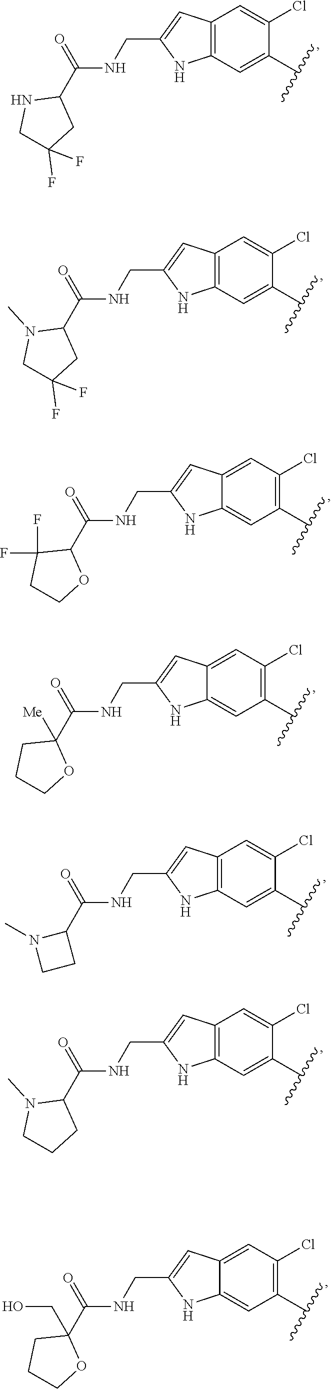

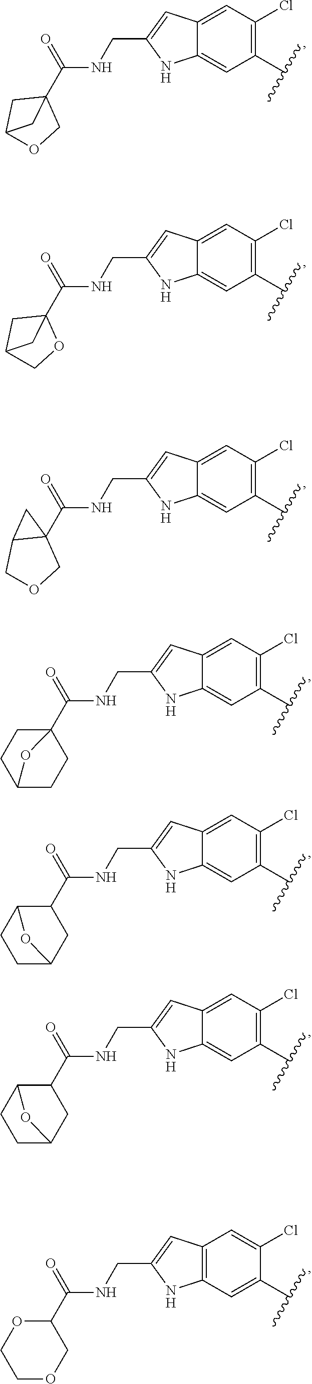

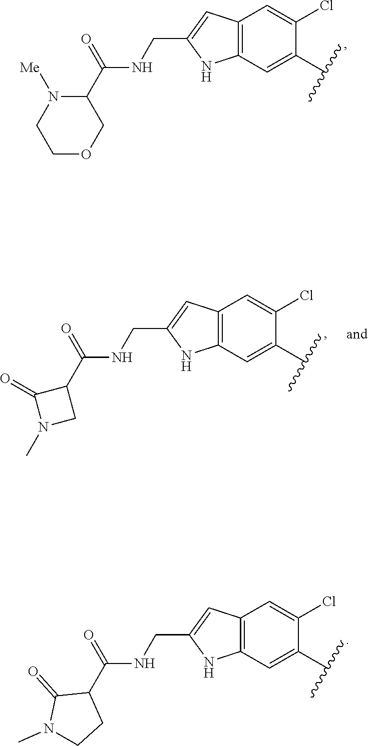 [SMILES: *c1cc2[nH]c(CNC(=O)C3COCCN3C)cc2cc1Cl ; *c1cc2[nH]c(CNC(=O)C3CN(C)C3=O)cc2cc1Cl ; *c1cc2[nH]c(CNC(=O)C3CCN(C)C3=O)cc2cc1Cl]

In some embodiments of a compound of formula (I′), or a stereoisomer or tautomer thereof, or a pharmaceutically acceptable salt of any of the foregoing, the moiety represented by

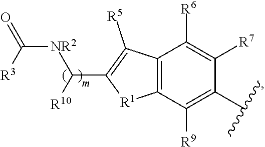

is selected from the group consisting of

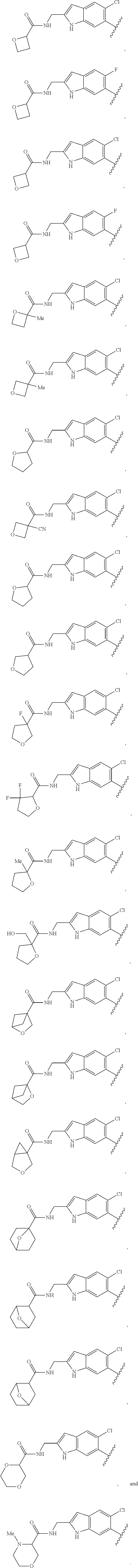

In some embodiments, the moiety represented by

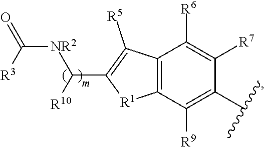

is selected from the group consisting of

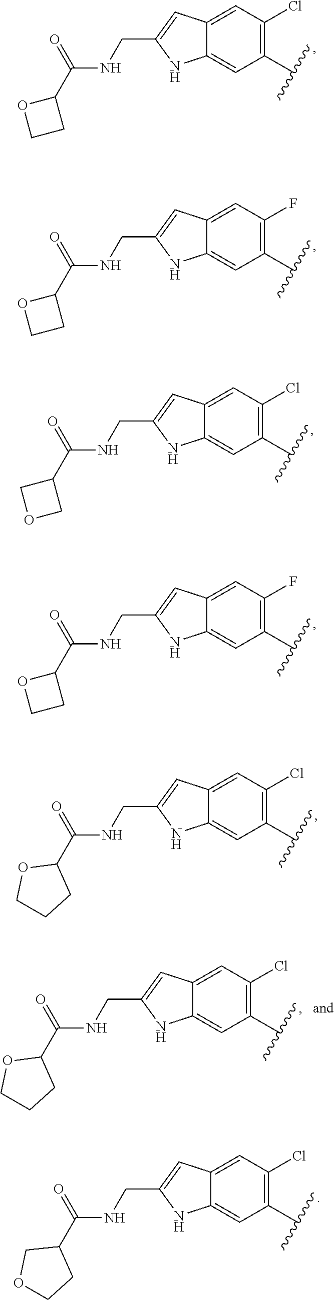

In some embodiments of a compound of formula (II), or a stereoisomer or tautomer thereof, or a pharmaceutically acceptable salt of any of the foregoing, the moiety represented by

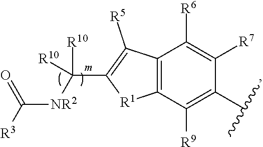

is

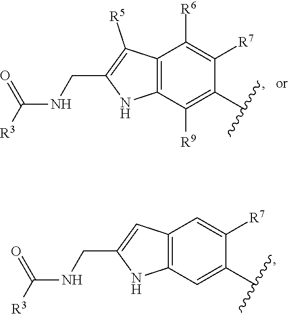

wherein R³ is 5-20 membered heteroaryl optionally substituted with one or more R^{a}. In some embodiments, R³ is 4-10 membered heterocyclyl optionally substituted with one or more R^{a}; R⁵ and R⁷ are each independently halo; and R⁶ and R⁹ are each H. In some embodiments, the moiety represented by

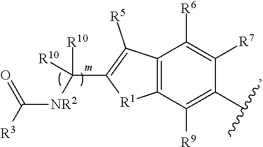

is selected from the group consisting of

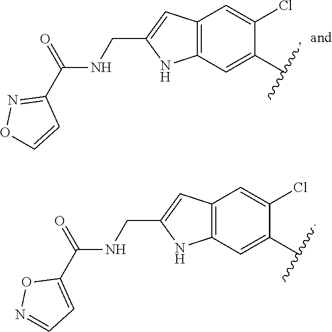

In some embodiments of a compound of formula (II), or a stereoisomer or tautomer thereof, or a pharmaceutically acceptable salt of any of the foregoing, the moiety represented by

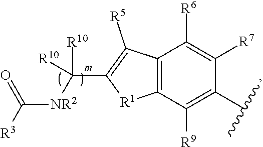

is

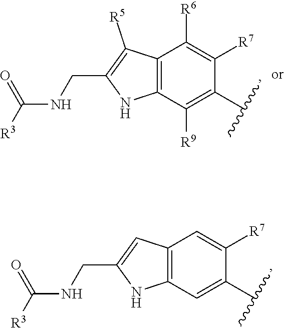

wherein R³ is —C(O)C₁₋₆alkyl. In some embodiments, R³ is —C(O)C₁₋₆alkyl; R⁵ and R⁷ are each independently halo; and R⁶ and R⁹ are each H. In some embodiments, the moiety represented by

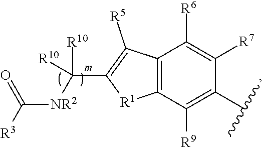

is

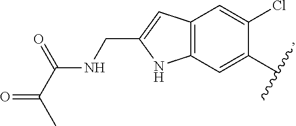

In some embodiments of a compound of formula (II), or a stereoisomer or tautomer thereof, or a pharmaceutically acceptable salt of any of the foregoing, the moiety represented by

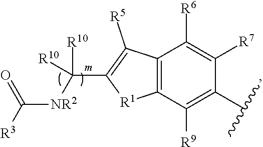

is

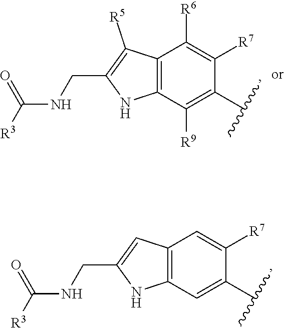

wherein R³ is C₁₋₆alkoxy. In some embodiments, R³ is C₁₋₆alkoxy; R⁵ and R⁷ are each independently halo; and R⁶ and R⁹ are each H. In some embodiments, the moiety represented by

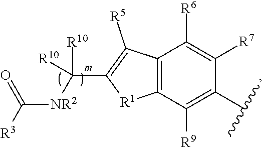

is selected from the group consisting of

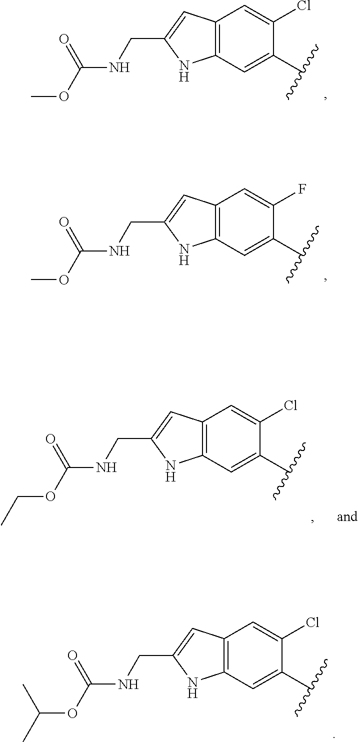 [SMILES: *c1cc2[nH]c(CNC(=O)OC)cc2cc1Cl ; *c1cc2[nH]c(CNC(=O)OC)cc2cc1F ; *c1cc2[nH]c(CNC(=O)OCC)cc2cc1Cl ; *c1cc2[nH]c(CNC(=O)OC(C)C)cc2cc1Cl]

In some embodiments of a compound of formula (II), or a stereoisomer or tautomer thereof, or a pharmaceutically acceptable salt of any of the foregoing, the moiety represented by

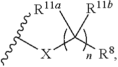

is

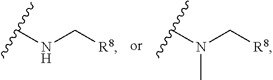

wherein R⁸ is C₆₋₂₀aryl. In some embodiments of a compound of formula (I), or a stereoisomer or tautomer thereof, or a pharmaceutically acceptable salt of any of the foregoing, the moiety represented by

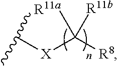

is selected from the group consisting of

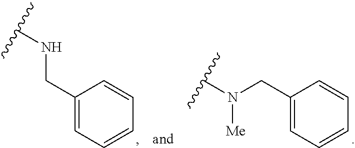 [SMILES: *NCc1ccccc1 ; *N(C)Cc1ccccc1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

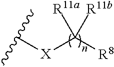

is

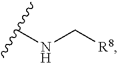

wherein R⁸ is 5-20 membered heteroaryl. In some embodiments of a compound of formula (I), or a stereoisomer or tautomer thereof, or a pharmaceutically acceptable salt of any of the foregoing, the moiety represented by

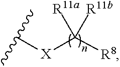

is selected from the group consisting of

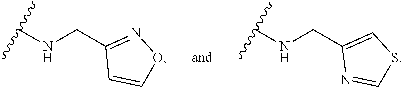 [SMILES: *NCc1ccon1 ; *NCc1cscn1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

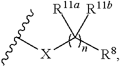

is

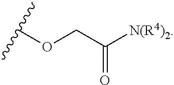

In some embodiments of a compound of formula (I), or a stereoisomer or tautomer thereof, or a pharmaceutically acceptable salt of any of the foregoing, the moiety represented by

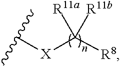

is

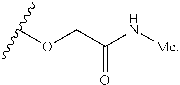 [SMILES: *OCC(=O)NC]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

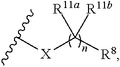

is

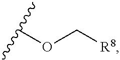

wherein R⁸ is C₆₋₂₀aryl optionally substituted with one or more R^{a}. In some embodiments of a compound of formula (I), or a stereoisomer or tautomer thereof, or a pharmaceutically acceptable salt of any of the foregoing, the moiety represented by

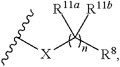

is selected from the group consisting of

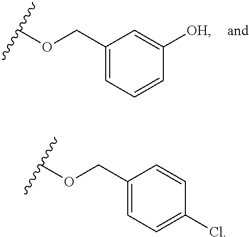 [SMILES: *OCc1cccc(O)c1 ; *OCc1ccc(Cl)cc1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

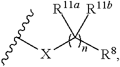

is

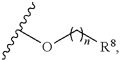

wherein n is an integer from 0 to 2; and R⁸ is C₃₋₁₀cycloalkyl optionally substituted with one or more R^{a}. In some embodiments, the moiety represented by

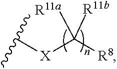

is selected from the group consisting of

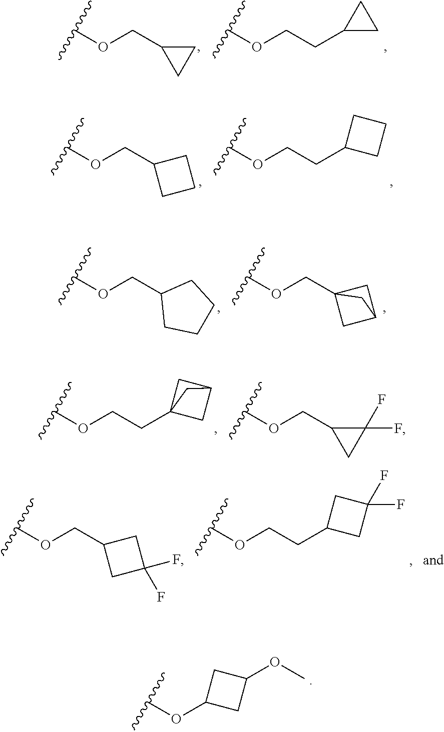

In some embodiments of a compound of formula (I), or a stereoisomer or tautomer thereof, or a pharmaceutically acceptable salt of any of the foregoing, the moiety represented by

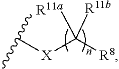

is selected from the group consisting of

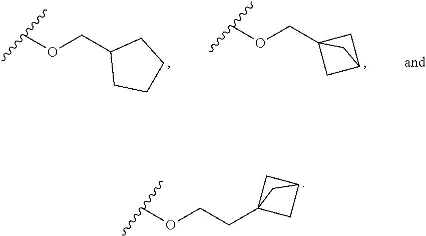 [SMILES: *OCC1CCCC1 ; *OCC12CC(C1)C2 ; *OCCC12CC(C1)C2]

In some variations, this embodiment also applies to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

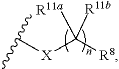

is

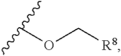

wherein R³ is C₆₋₂₀aryl. In some embodiments of a compound of formula (I), or a stereoisomer or tautomer thereof, or a pharmaceutically acceptable salt of any of the foregoing, the moiety represented by

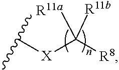

is

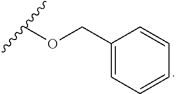 [SMILES: *OCc1ccccc1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

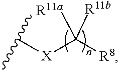

is

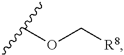

wherein R⁸ is 3-10 membered heterocyclyl. In some embodiments, the moiety represented by

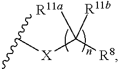

is selected from the group consisting of

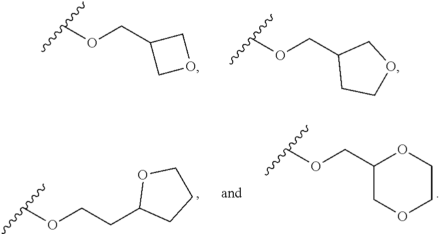 [SMILES: *OCC1COC1 ; *OCC1CCOC1 ; *OCCC1CCCO1 ; *OCC1COCCO1]

In some embodiments of a compound of formula (I), or a stereoisomer or tautomer thereof, or a pharmaceutically acceptable salt of any of the foregoing, the moiety represented by

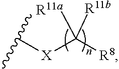

is

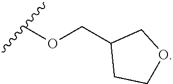 [SMILES: *OCC1CCOC1]

In some variations, this embodiment also applies to any other applicable formula detailed herein, such as a compound of formula (I′) or (II) or a stereoisomer or
tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

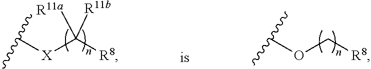

wherein n is 1 or 2; and R⁸ is 5-20 membered heteroaryl optionally substituted with one or more R^{a}. In some embodiments, the moiety represented by

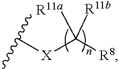

is selected from the group consisting of

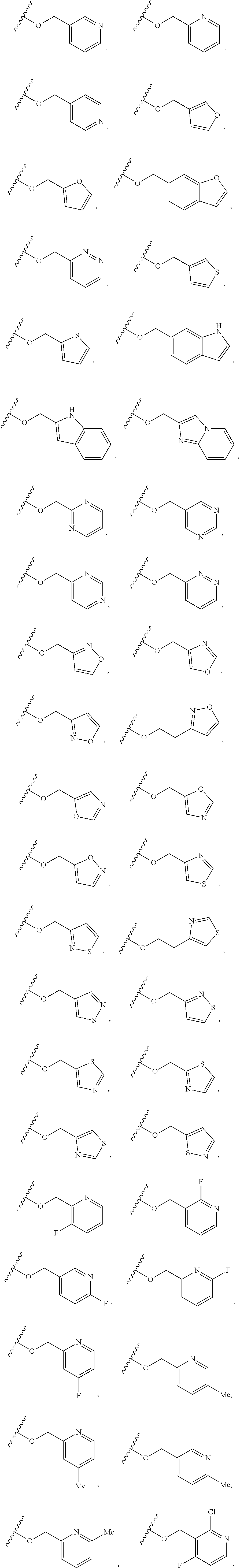

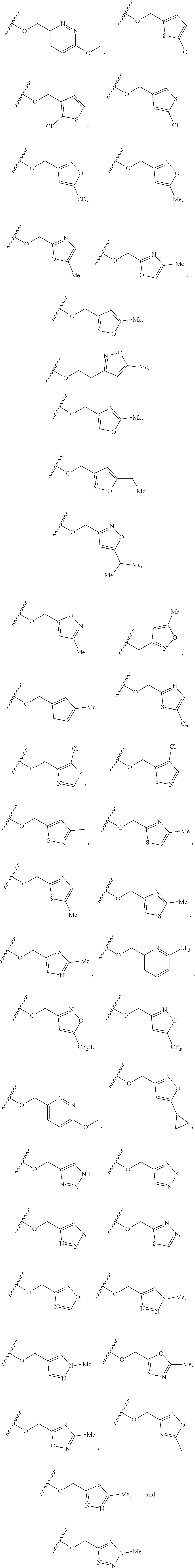

In some embodiments of a compound of formula (I), or a stereoisomer or tautomer thereof, or a pharmaceutically acceptable salt of any of the foregoing, the moiety represented by

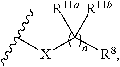

is selected from the group consisting of

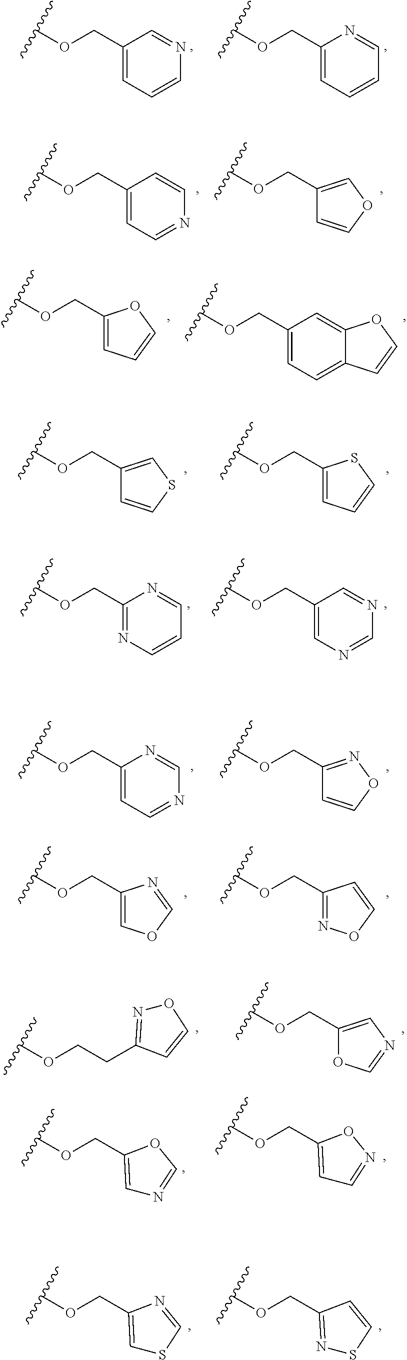

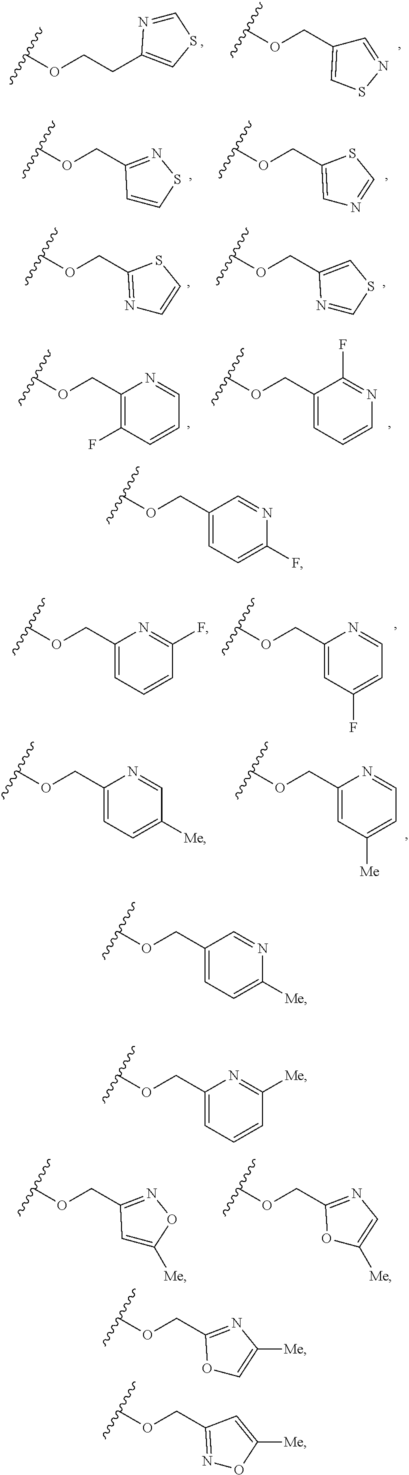

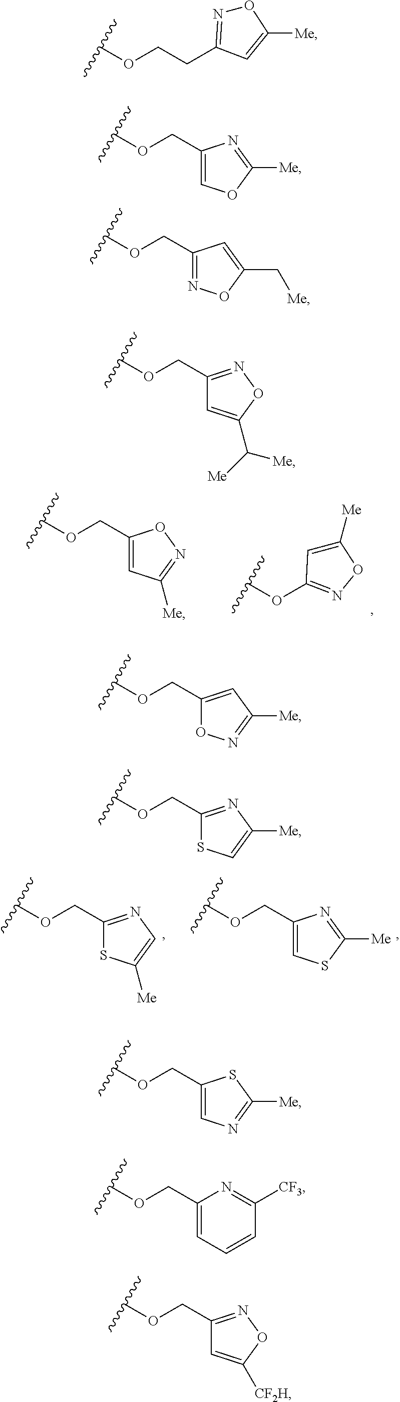

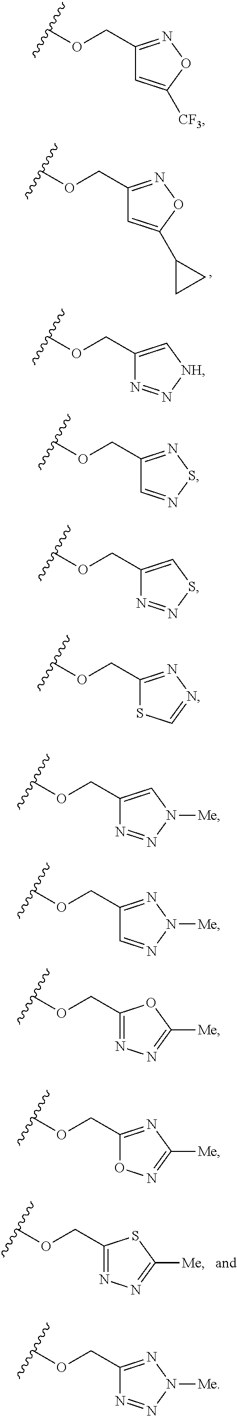

In some embodiments, the moiety represented by

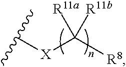

is selected from the group consisting of

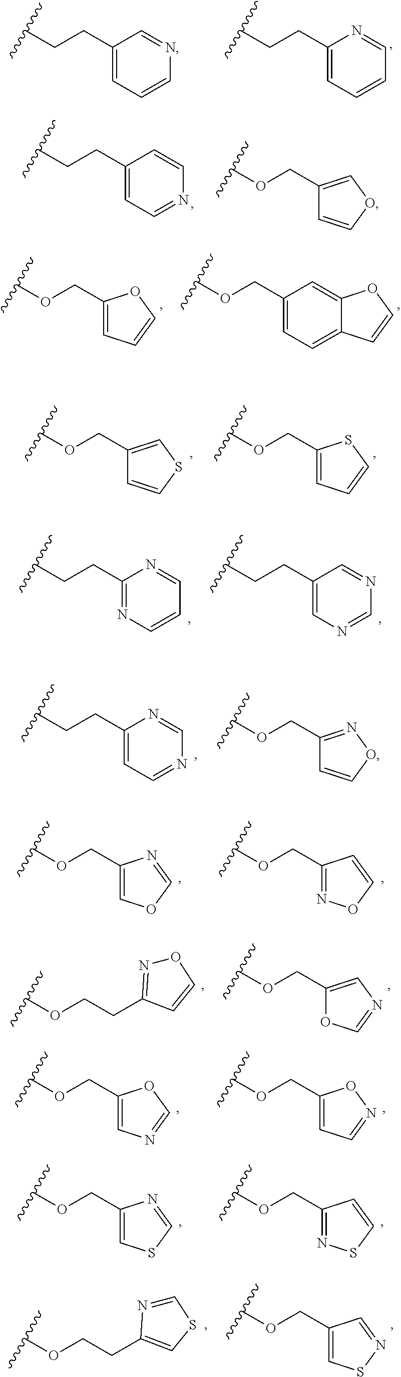

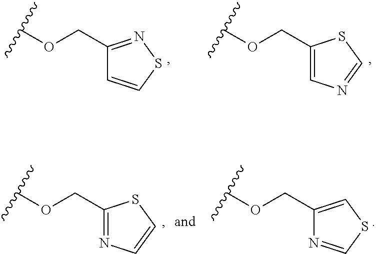

In some embodiments, the moiety represented by

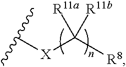

is selected from the group consisting of

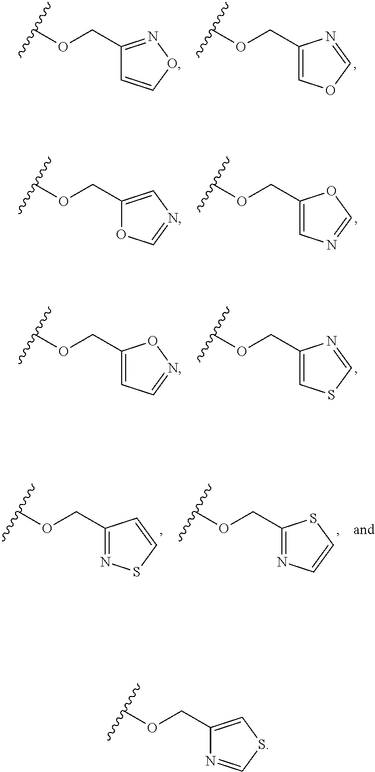

In some embodiments, the moiety represented by

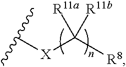

is selected from the group consisting of

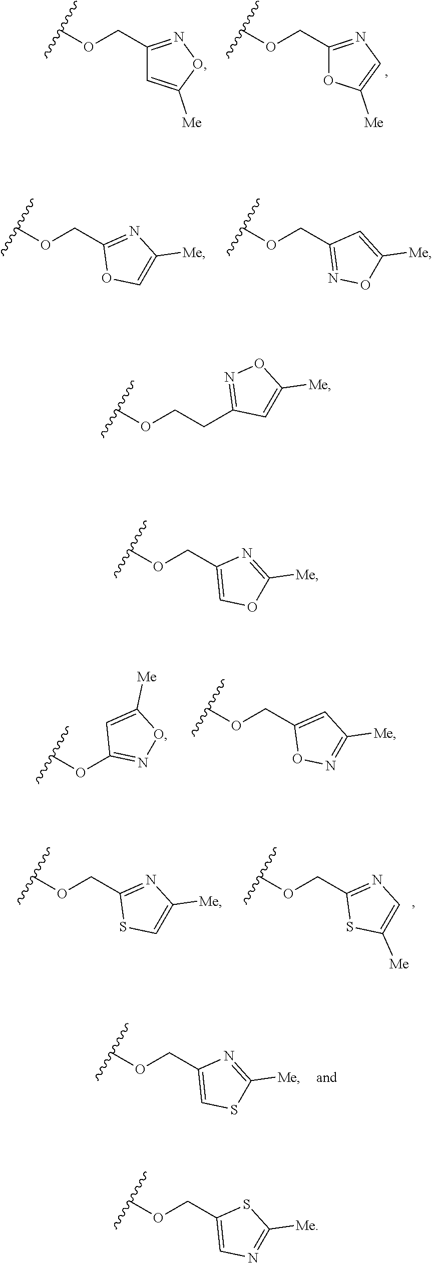

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or a stereoisomer or tautomer thereof, or a pharmaceutically acceptable salt of any of the foregoing, the moiety represented by

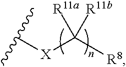

is selected from the group consisting of

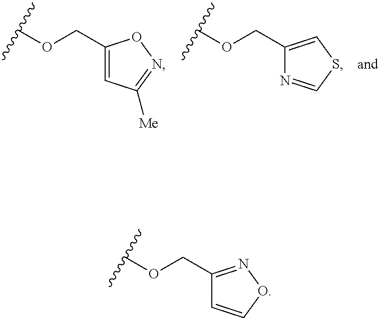 [SMILES: *OCc1cc(C)no1 ; *OCc1cscn1 ; *OCc1ccon1]

In some embodiments of a compound of formula (II), or a stereoisomer or tautomer thereof, or a pharmaceutically acceptable salt of any of the foregoing, the moiety represented by

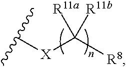

is

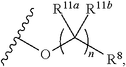

wherein n is 2; and R⁸ is C₁₋₆alkoxy, or —O—C₃₋₁₀cycloalkyl, wherein each R⁸ is optionally substituted with one or more R^{a}. In some embodiments, the moiety represented by

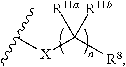

is selected from the group consisting of

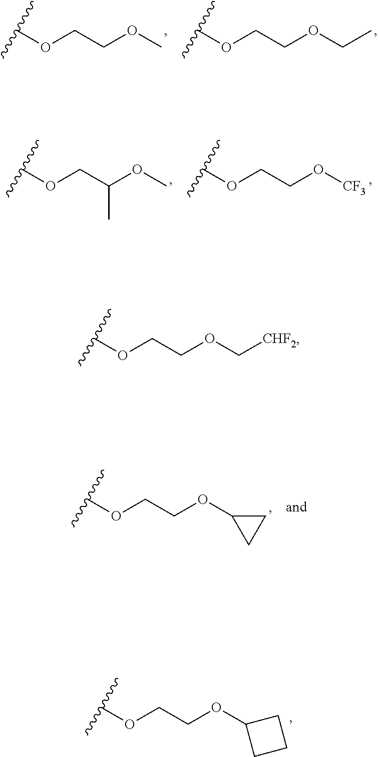

In some embodiments of a compound of formula (II), or a stereoisomer or tautomer thereof, or a pharmaceutically acceptable salt of any of the foregoing, the moiety represented by

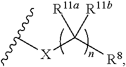

is

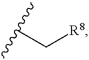

wherein R⁸ is —(R^{x})-5-20 membered heteroaryl); and R^{x} is —NH— or —O—. In some embodiments, the moiety represented by

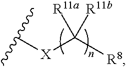

is selected from the group consisting of

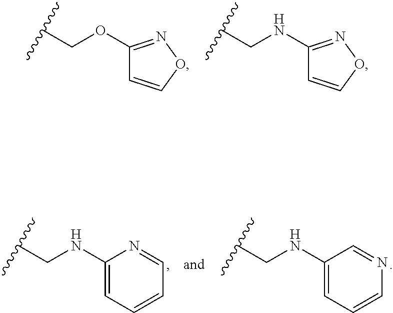 [SMILES: *COc1ccon1 ; *CNc1ccon1 ; *CNc1ccccn1 ; *CNc1cccnc1]

In some embodiments of a compound of formula (II), or a stereoisomer or tautomer thereof, or a pharmaceutically acceptable salt of any of the foregoing, the moiety represented by

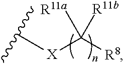

is

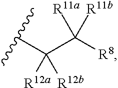

wherein 5-20 membered heteroaryl optionally substituted with one or more R^{a}; R^{11a} and R^{11b} are each independently H or C₁₋₆alkyl; and R^{12a} and R^{12b} are each independently, H or C₁₋₆alkyl. In some embodiments of a compound of formula (I), or a stereoisomer or tautomer thereof, or a pharmaceutically acceptable salt of any of the foregoing, the moiety represented by

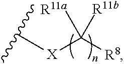

is selected from the group consisting of

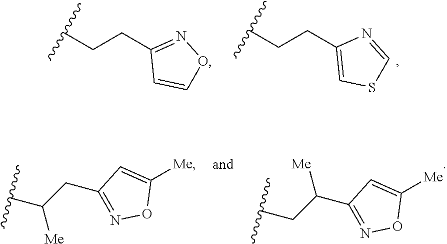 [SMILES: *CCc1ccon1 ; *CCc1cscn1 ; *C(C)Cc1cc(C)on1 ; *CC(C)c1cc(C)on1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

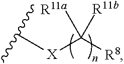

is

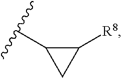

wherein R⁸ is C₆₋₂₀aryl. In some embodiments of a compound of formula (I), or a stereoisomer or tautomer thereof, or a pharmaceutically acceptable salt of any of the foregoing, the moiety represented by

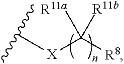

is

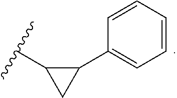 [SMILES: *C1CC1c1ccccc1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

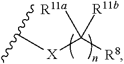

is

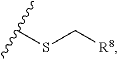

wherein R⁸ is 5-20 membered heteroaryl optionally substituted with one or more R^{a}. In some embodiments of a compound of formula (I), or a stereoisomer or tautomer thereof, or a pharmaceutically acceptable salt of any of the foregoing, the moiety represented by

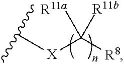

is

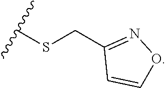 [SMILES: *SCc1ccon1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

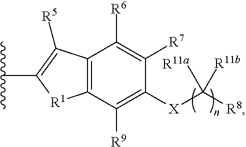

is

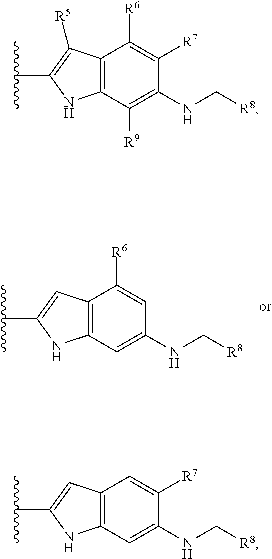

wherein R⁸ is 5-20 membered heteroaryl optionally substituted with one or more R^{a}. In some embodiments, R⁸ is 5-20 membered heteroaryl optionally substituted with one or more R^{a}; R⁵ and R⁷ are each independently H or halo; and R⁶ and R⁹ are each H or halo. In some embodiments, the moiety represented by

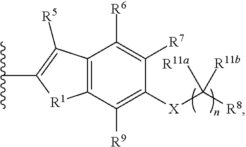

is selected from the group consisting of

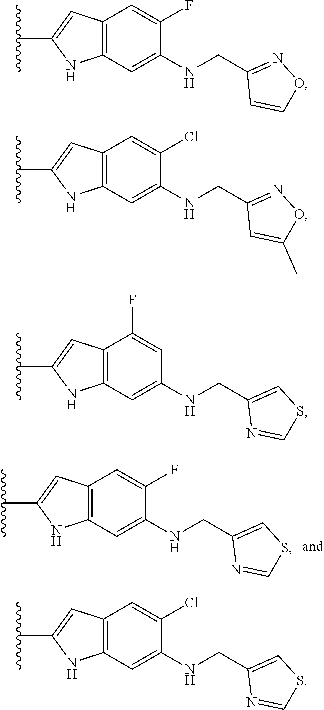 [SMILES: *c1cc2cc(F)c(NCc3ccon3)cc2[nH]1 ; *c1cc2cc(Cl)c(NCc3cc(C)on3)cc2[nH]1 ; *c1cc2c(F)cc(NCc3cscn3)cc2[nH]1 ; *c1cc2cc(F)c(NCc3cscn3)cc2[nH]1 ; *c1cc2cc(Cl)c(NCc3cscn3)cc2[nH]1]

In some embodiments of a compound of formula (II), or a stereoisomer or tautomer thereof, or a pharmaceutically acceptable salt of any of the foregoing, the moiety represented by

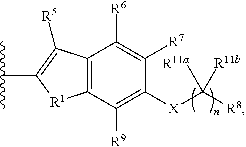

is

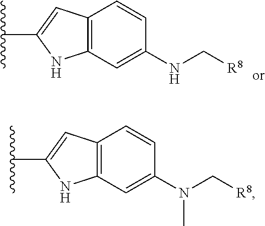

wherein R⁸ is C₆₋₂₀aryl. In some embodiments of a compound of formula (I), or a stereoisomer or tautomer thereof, or a pharmaceutically acceptable salt of any of the foregoing, the moiety represented by

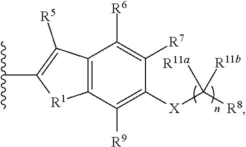

is selected from the group consisting of

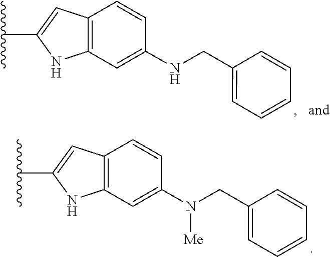 [SMILES: *c1cc2ccc(NCc3ccccc3)cc2[nH]1 ; *c1cc2ccc(N(C)Cc3ccccc3)cc2[nH]1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, the moiety represented by

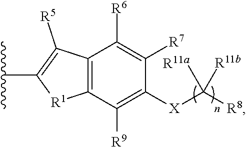

is

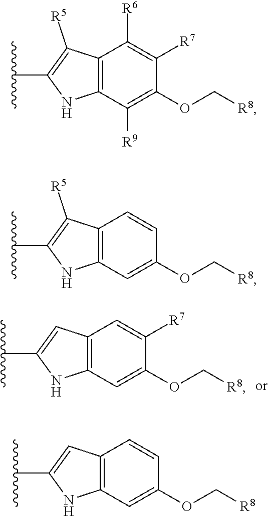

wherein R⁸ is C₆₋₂₀aryl optionally substituted with one or more R^{a}. In some embodiments of a compound of formula (I), or a stereoisomer or tautomer thereof, or a pharmaceutically acceptable salt of any of the foregoing, the moiety represented by

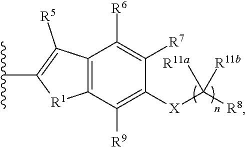

is selected from the group consisting of

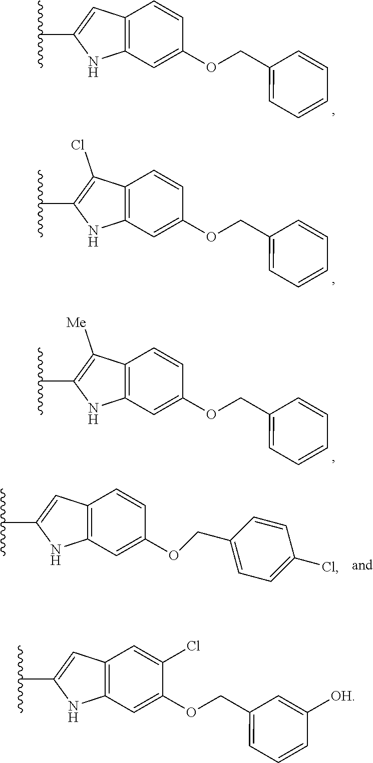 [SMILES: *c1cc2ccc(OCc3ccccc3)cc2[nH]1 ; *c1[nH]c2cc(OCc3ccccc3)ccc2c1Cl ; *c1[nH]c2cc(OCc3ccccc3)ccc2c1C ; *c1cc2ccc(OCc3ccc(Cl)cc3)cc2[nH]1 ; *c1cc2cc(Cl)c(OCc3cccc(O)c3)cc2[nH]1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

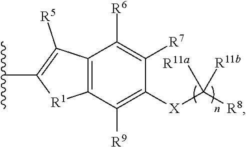

is

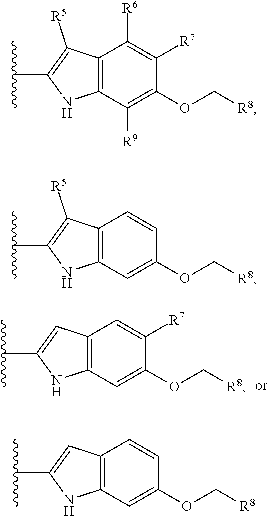

wherein R⁸ is 5-20 membered heteroaryl optionally substituted with one or more R^{a}. In some embodiments, the moiety represented by

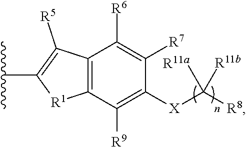

is selected from the group consisting of

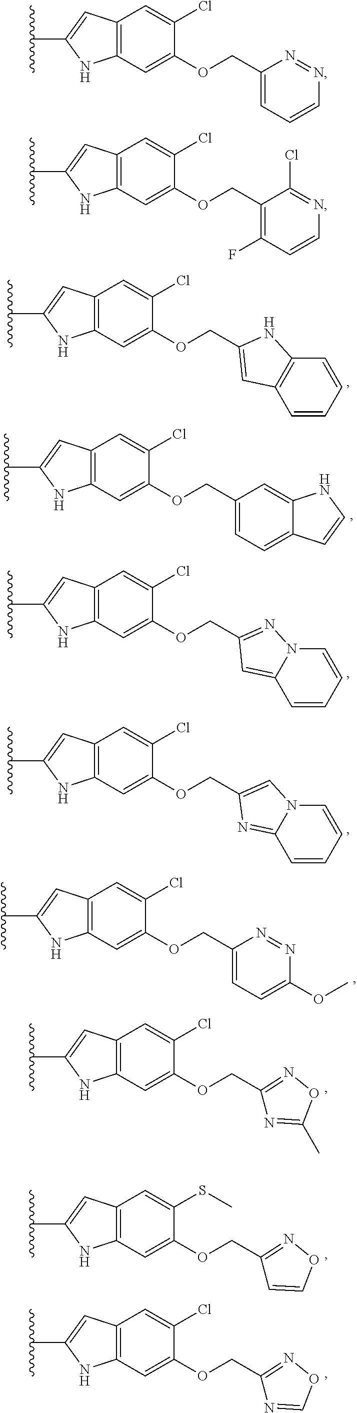

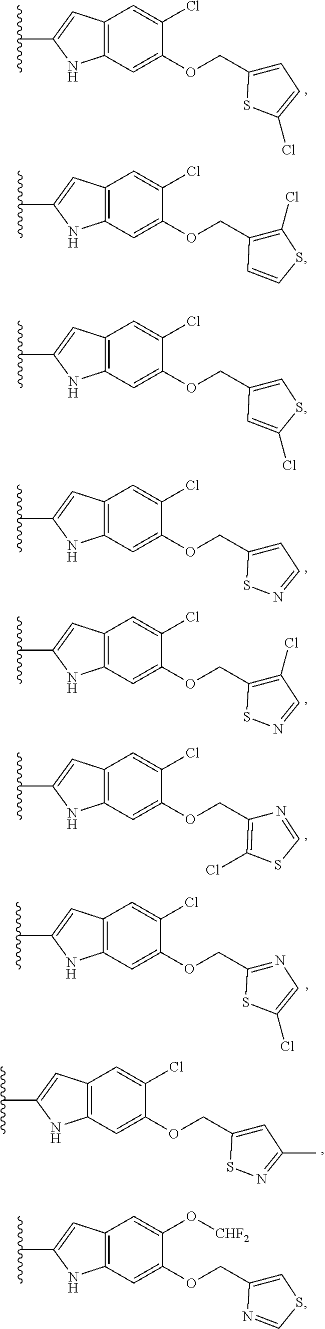

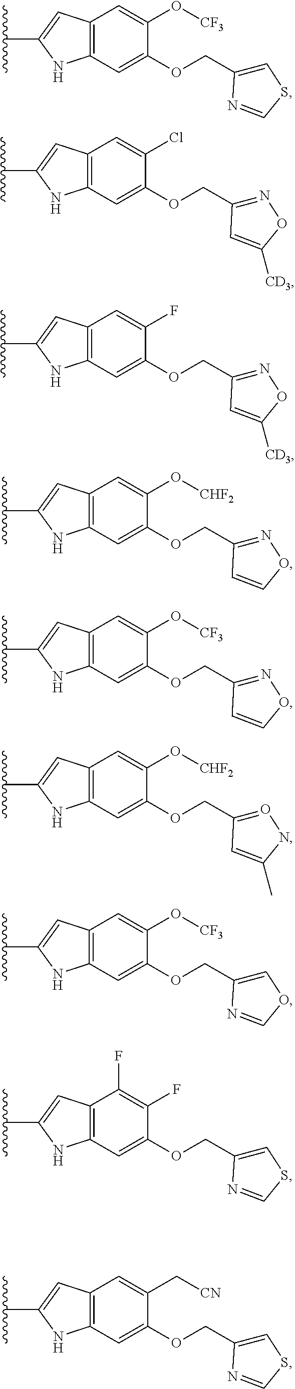

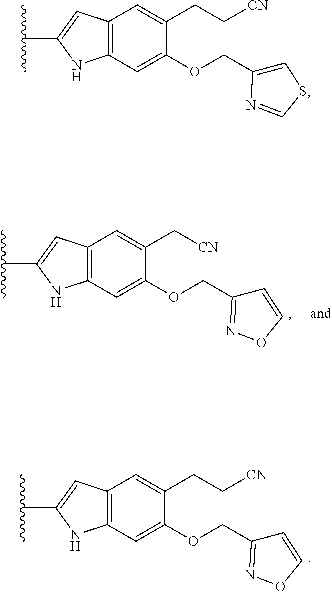 [SMILES: *c1cc2cc(CCC#N)c(OCc3cscn3)cc2[nH]1 ; *c1cc2cc(CC#N)c(OCc3ccon3)cc2[nH]1 ; *c1cc2cc(CCC#N)c(OCc3ccon3)cc2[nH]1]

In some embodiments of a compound of formula (I), or a stereoisomer or tautomer thereof, or a pharmaceutically acceptable salt of any of the foregoing, the moiety represented by

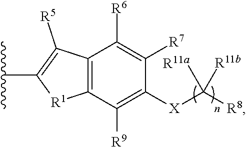

is selected from the group consisting of

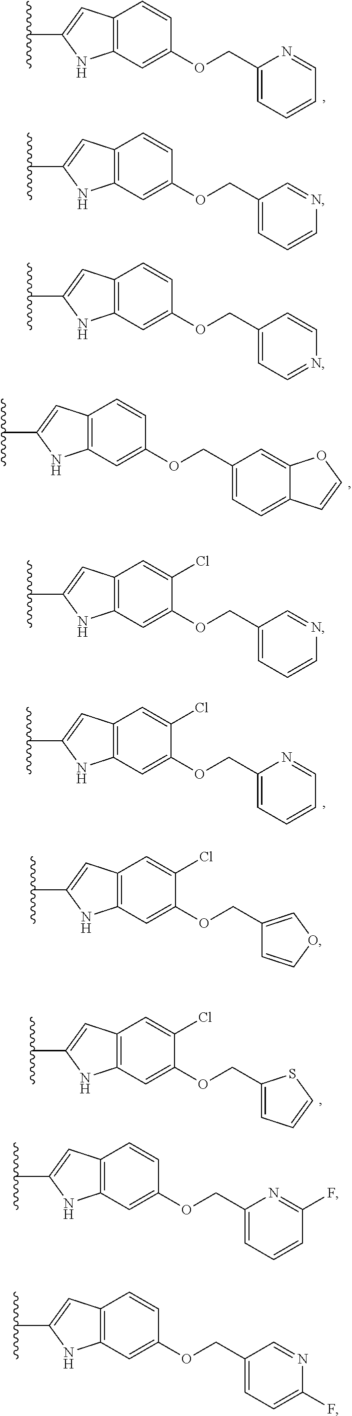

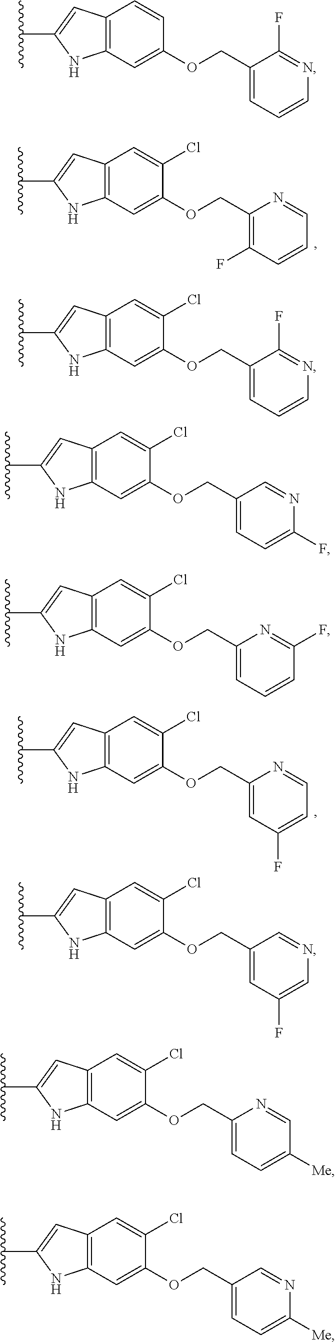

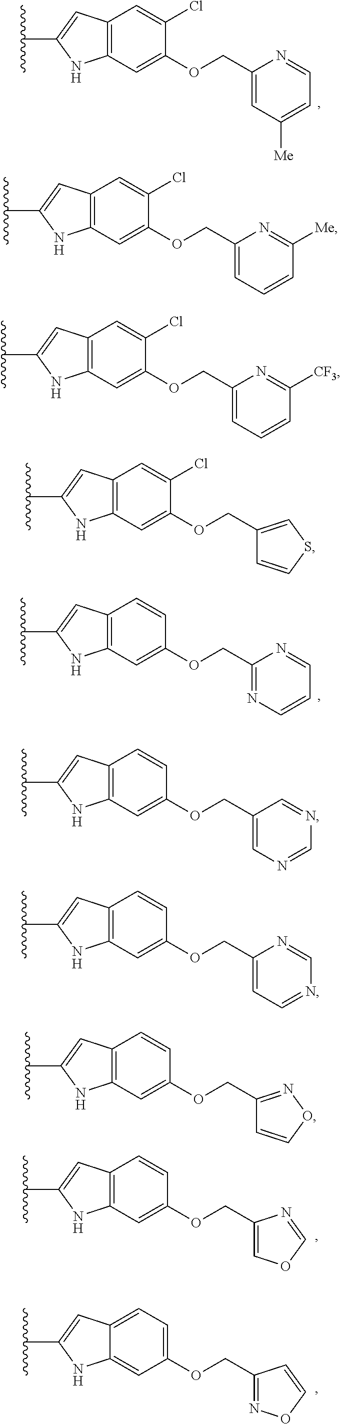

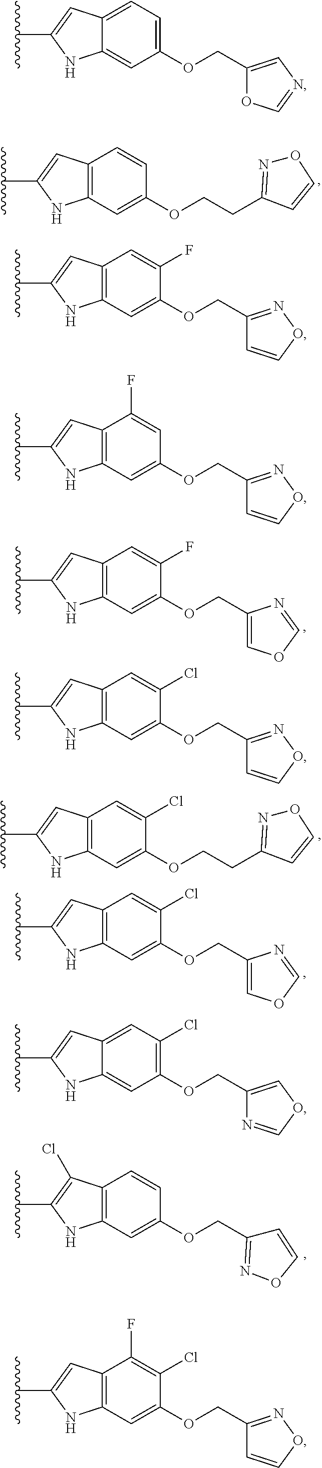

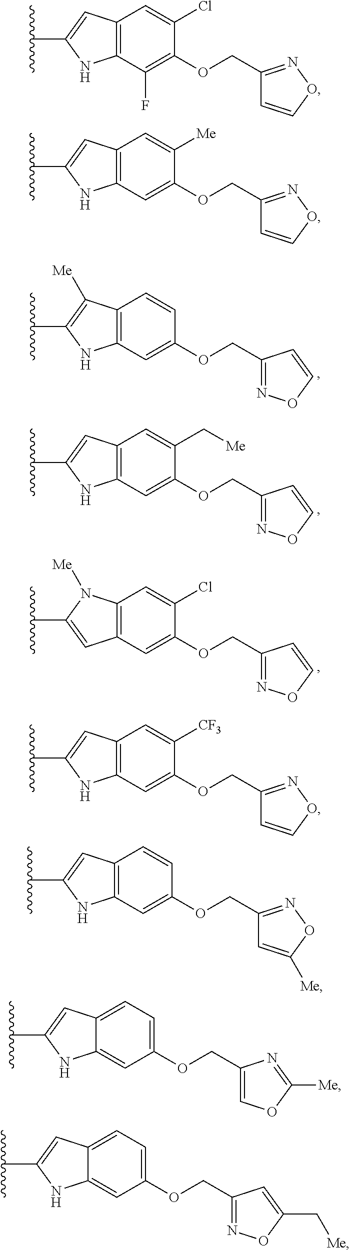

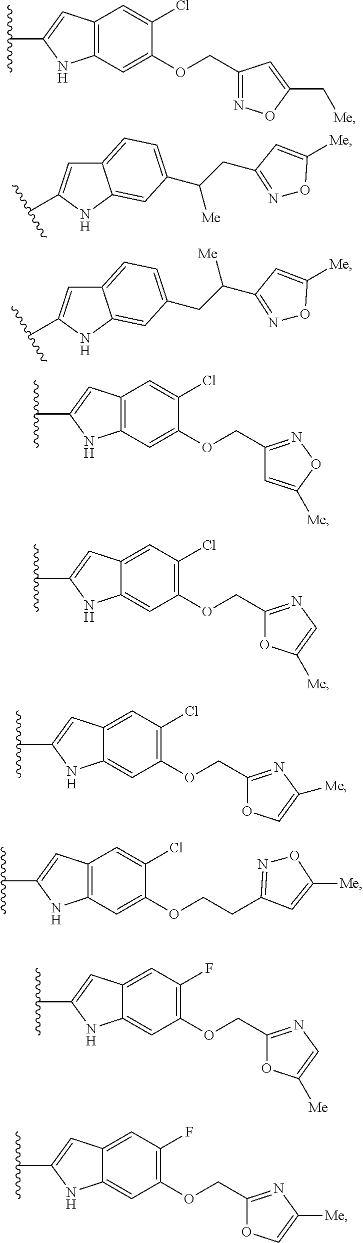

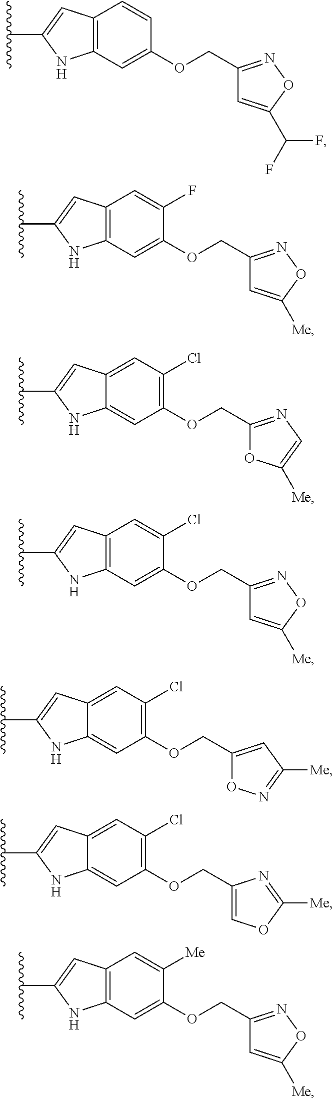

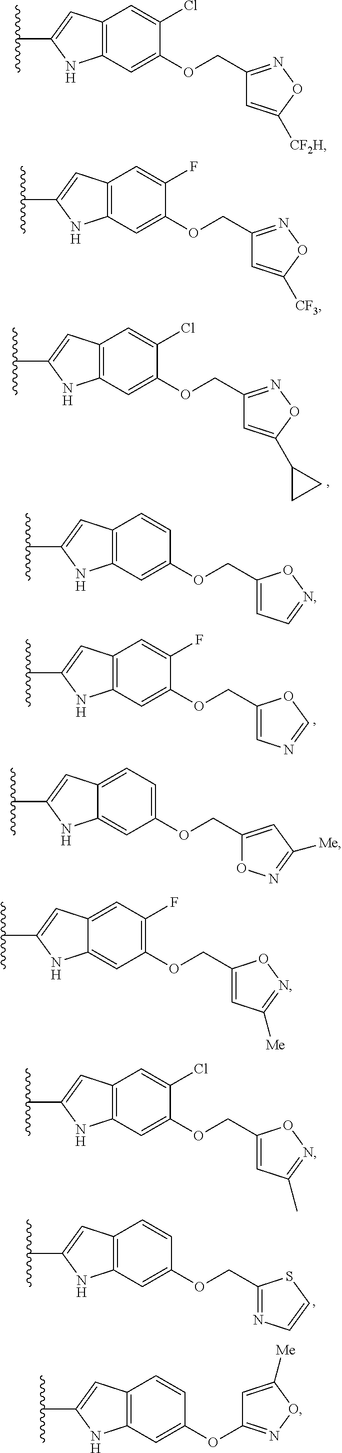

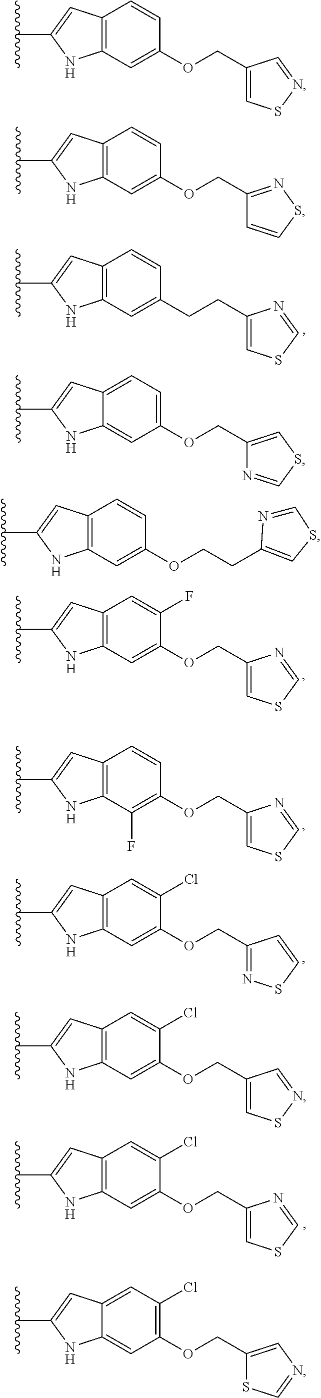

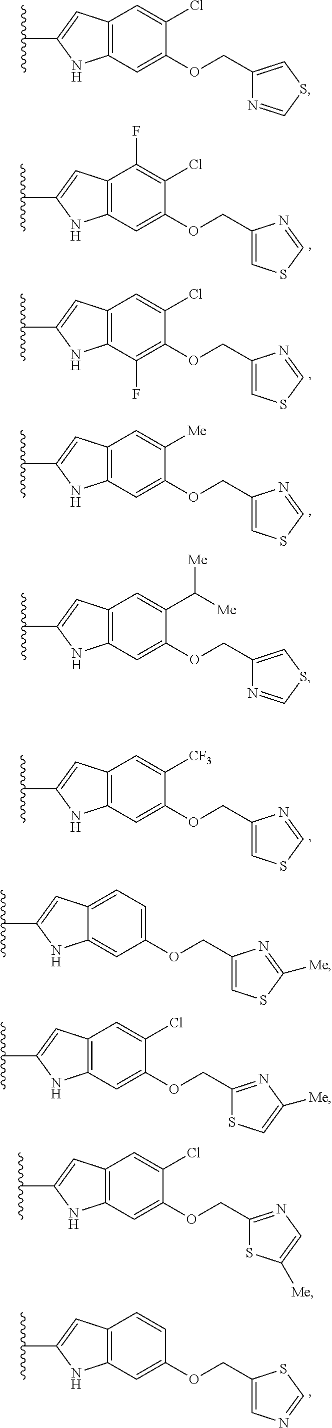

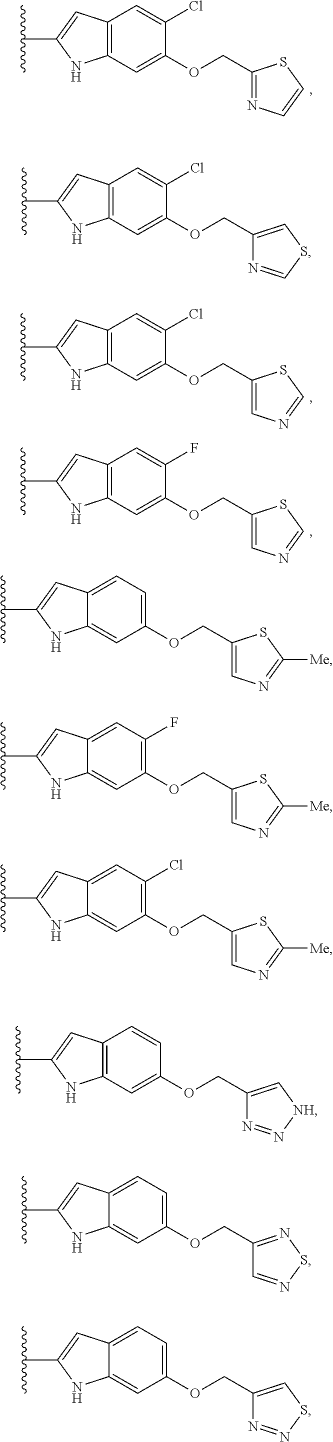

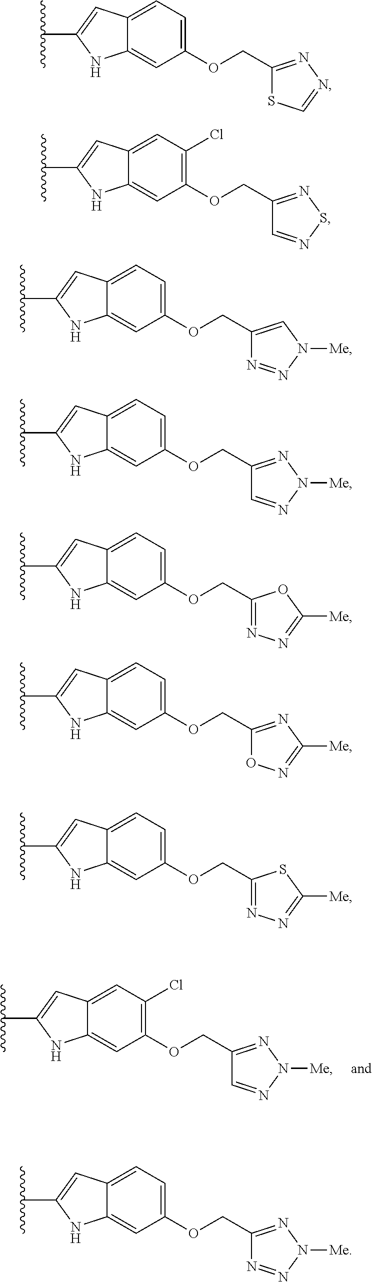

Additionally, the moiety represented by

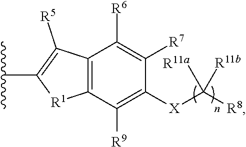

may be selected from the group consisting of

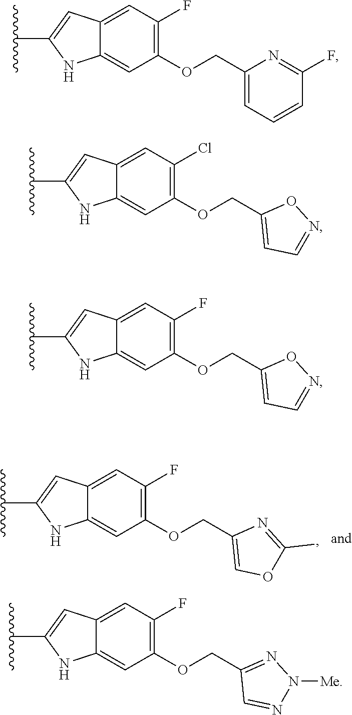

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

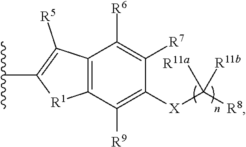

is

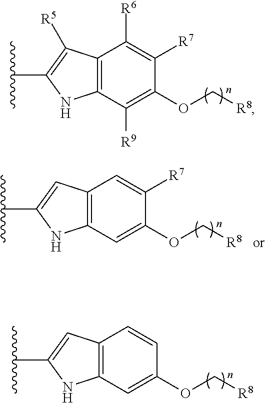

wherein n is 0 or 1; and R⁸ is C₃₋₁₀cycloalkyl optionally substituted with one or more R^{a}. In some embodiments, the moiety represented by

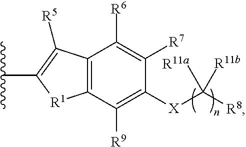

is selected from the group consisting of

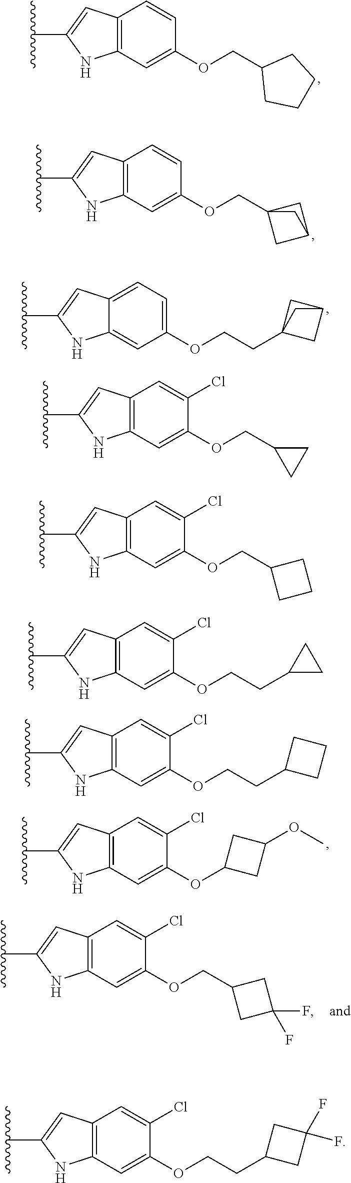

In some embodiments of a compound of formula (I), or a stereoisomer or tautomer thereof, or a pharmaceutically acceptable salt of any of the foregoing, the moiety represented by

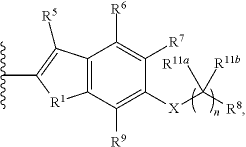

is selected from the group consisting of

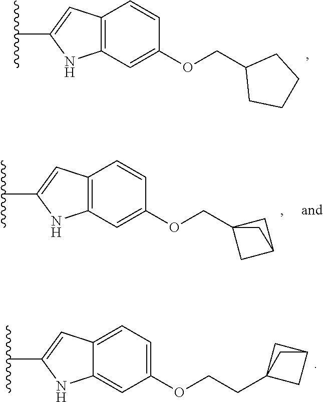 [SMILES: *c1cc2ccc(OCC3CCCC3)cc2[nH]1 ; *c1cc2ccc(OCC34CC(C3)C4)cc2[nH]1 ; *c1cc2ccc(OCCC34CC(C3)C4)cc2[nH]1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

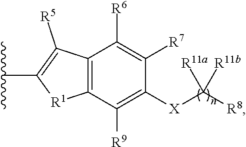

is

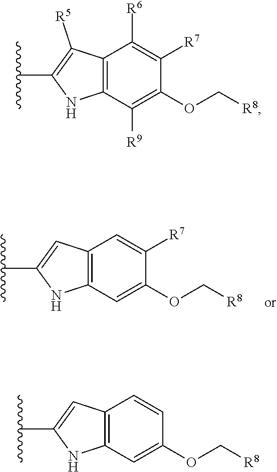

wherein R⁸ is C₃₋₁₀cycloakyl. In some embodiments, the moiety represented by

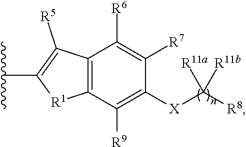

is selected from the group consisting of

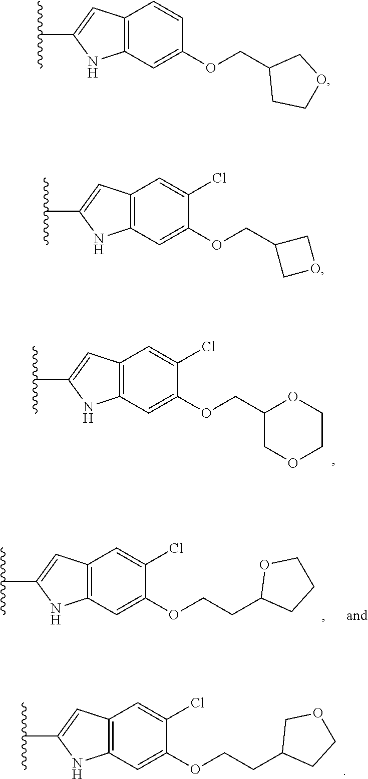 [SMILES: *c1cc2ccc(OCC3CCOC3)cc2[nH]1 ; *c1cc2cc(Cl)c(OCC3COC3)cc2[nH]1 ; *c1cc2cc(Cl)c(OCC3COCCO3)cc2[nH]1 ; *c1cc2cc(Cl)c(OCCC3CCCO3)cc2[nH]1 ; *c1cc2cc(Cl)c(OCCC3CCOC3)cc2[nH]1]

In some embodiments of a compound of formula (I), or a stereoisomer or tautomer thereof or a pharmaceutically acceptable salt of any of the foregoing, the moiety represented by

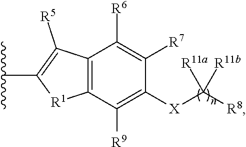

is

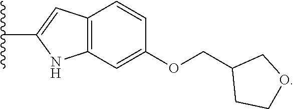 [SMILES: *c1cc2ccc(OCC3CCOC3)cc2[nH]1]

In some variations, this embodiment also applies to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, the moiety represented by

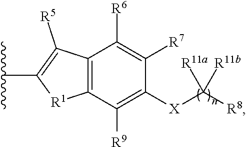

is

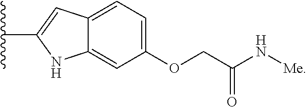 [SMILES: *c1cc2ccc(OCC(=O)NC)cc2[nH]1]

In some variations, this embodiment also applies to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

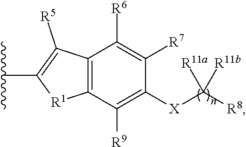

is

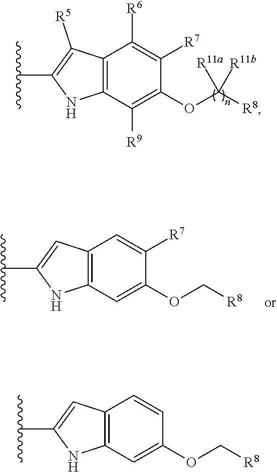

wherein n is 2; and R⁸ is C₁₋₆alkoxy, or —O—C₃₋₁₀cycloalkyl, wherein each R⁸ is optionally substituted with one or more R^{a}. In some embodiments, the moiety represented by

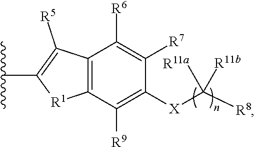

is selected from the group consisting of

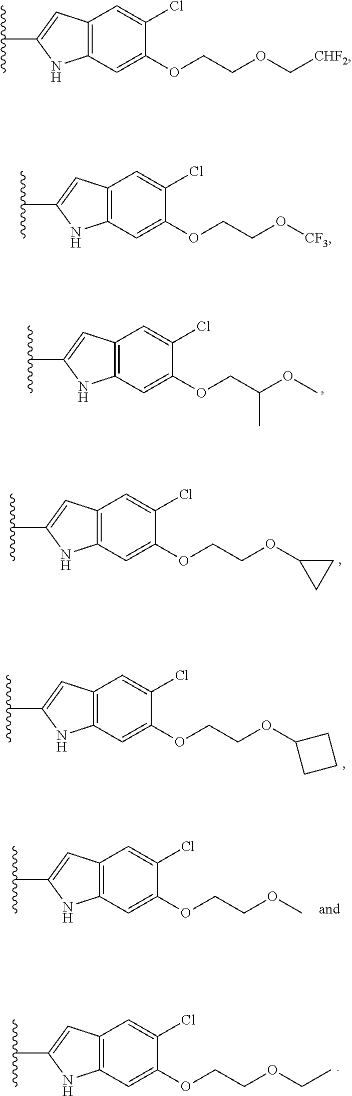

In some embodiments of a compound of formula (II), or a stereoisomer or tautomer thereof, or a pharmaceutically acceptable salt of any of the foregoing, the moiety represented by

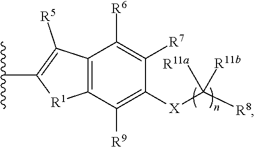

is

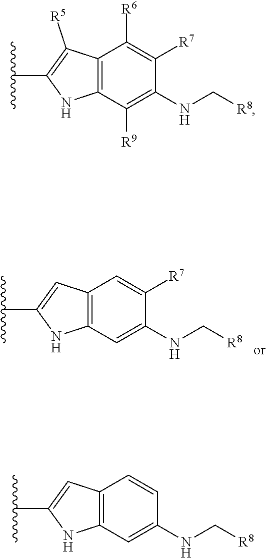

wherein R⁸ is 5-20 membered heteroaryl. In some embodiments of a compound of formula (I), or a stereoisomer or tautomer thereof, or a pharmaceutically acceptable salt of any of the foregoing, the moiety represented by

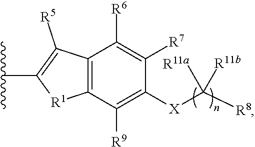

is selected from the group consisting of

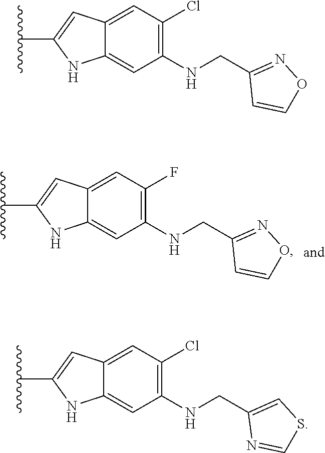 [SMILES: *c1cc2cc(Cl)c(NCc3ccon3)cc2[nH]1 ; *c1cc2cc(F)c(NCc3ccon3)cc2[nH]1 ; *c1cc2cc(Cl)c(NCc3cscn3)cc2[nH]1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

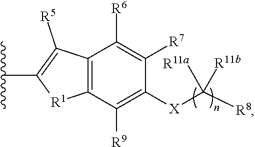

is

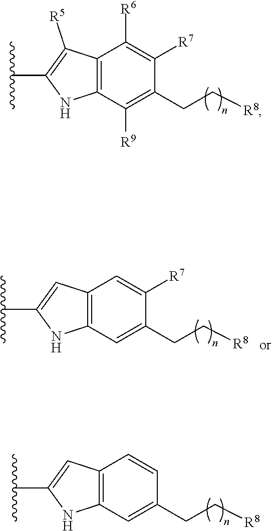

wherein n is 0 or 1, and R⁸ is 5-20 membered heteroaryl. In some embodiments, the moiety represented by

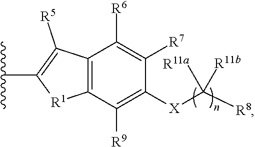

is selected from the group consisting of

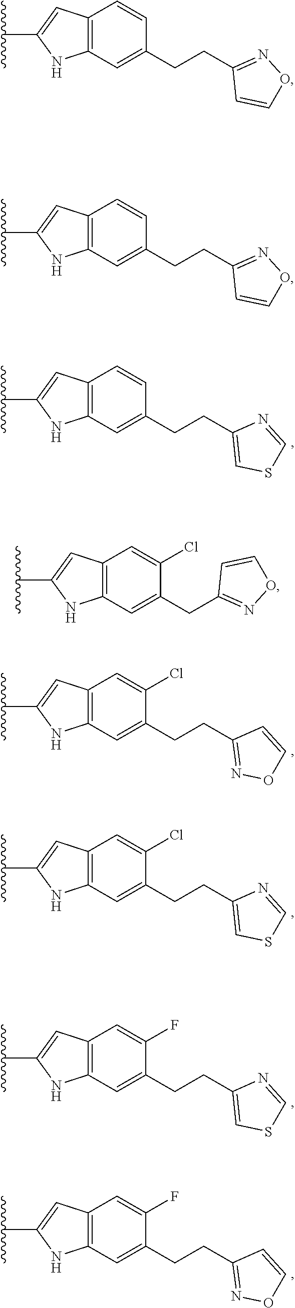

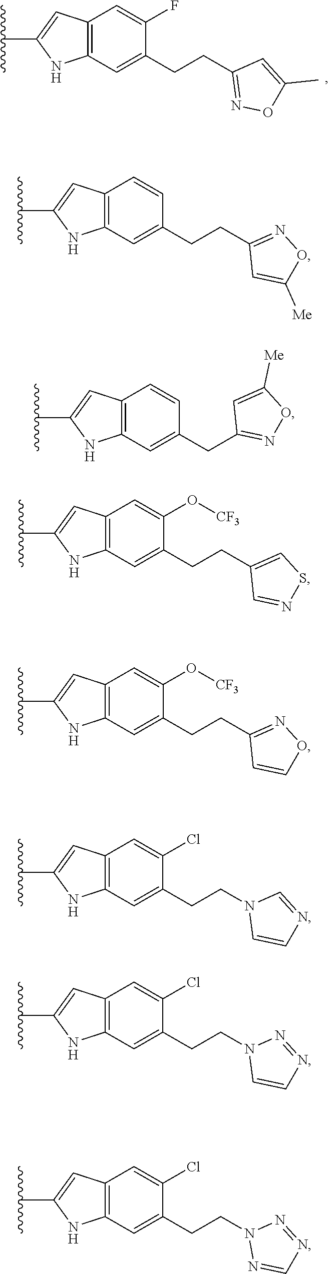

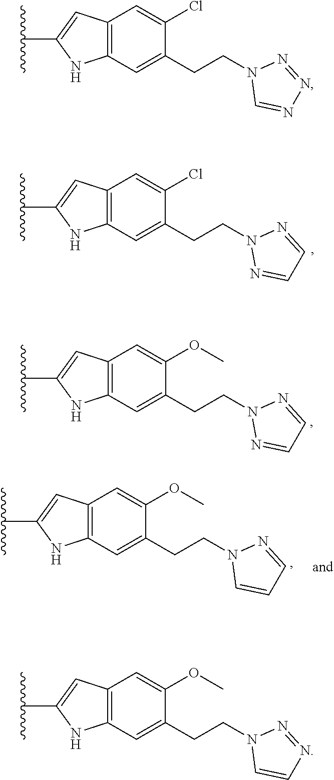 [SMILES: *c1cc2cc(Cl)c(CCn3cnnn3)cc2[nH]1 ; *c1cc2cc(Cl)c(CCn3nccn3)cc2[nH]1 ; *c1cc2cc(OC)c(CCn3nccn3)cc2[nH]1 ; *c1cc2cc(OC)c(CCn3cccn3)cc2[nH]1 ; *c1cc2cc(OC)c(CCn3ccnn3)cc2[nH]1]

In some embodiments of a compound of formula (I), or a stereoisomer or tautomer thereof, or a pharmaceutically acceptable salt of any of the foregoing, the moiety represented by

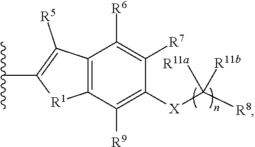

is selected from the group consisting of

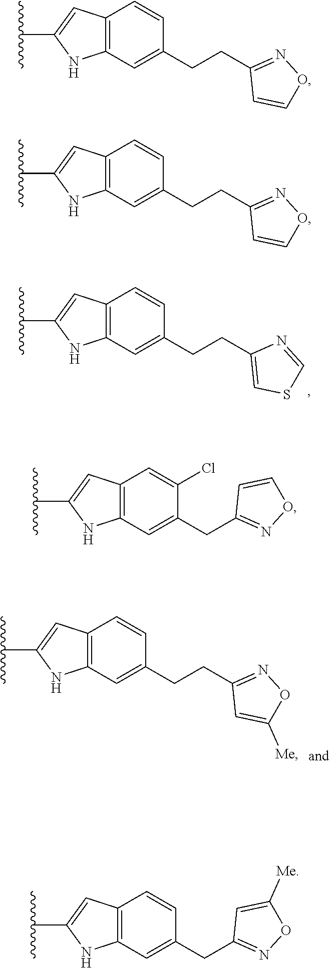 [SMILES: *c1cc2ccc(CCc3ccon3)cc2[nH]1 ; *c1cc2ccc(CCc3ccon3)cc2[nH]1 ; *c1cc2ccc(CCc3cscn3)cc2[nH]1 ; *c1cc2cc(Cl)c(Cc3ccon3)cc2[nH]1 ; *c1cc2ccc(CCc3cc(C)on3)cc2[nH]1 ; *c1cc2ccc(Cc3cc(C)on3)cc2[nH]1]

In some variations, this embodiment also applies to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

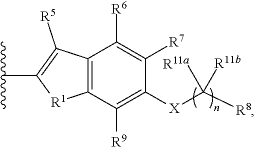

is

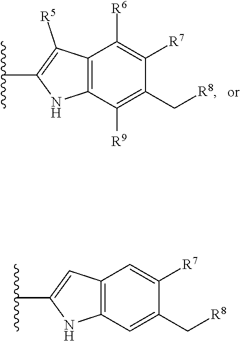

wherein R⁸ is —(R^{x})-5-20 membered heteroaryl); and R^{x} is —NH— or —O—. In some embodiments, the moiety represented by

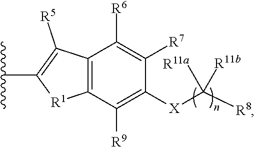

is selected from the group consisting of

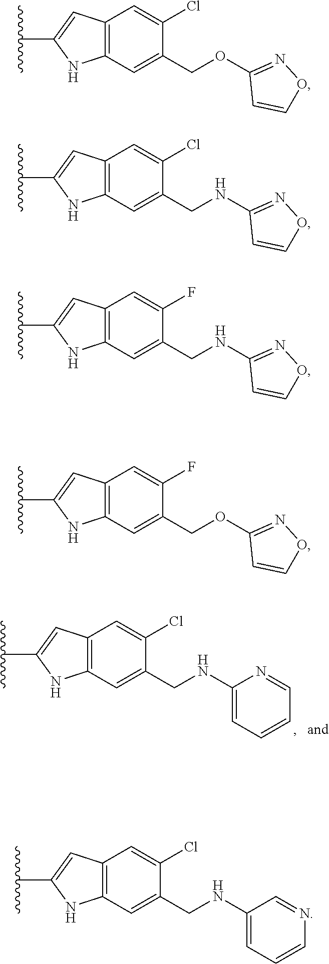 [SMILES: *c1cc2cc(Cl)c(COc3ccon3)cc2[nH]1 ; *c1cc2cc(Cl)c(CNc3ccon3)cc2[nH]1 ; *c1cc2cc(F)c(CNc3ccon3)cc2[nH]1 ; *c1cc2cc(F)c(COc3ccon3)cc2[nH]1 ; *c1cc2cc(Cl)c(CNc3ccccn3)cc2[nH]1 ; *c1cc2cc(Cl)c(CNc3cccnc3)cc2[nH]1]

In some embodiments of a compound of formula (II), or a stereoisomer or tautomer thereof, or a pharmaceutically acceptable salt of any of the foregoing, the moiety represented by

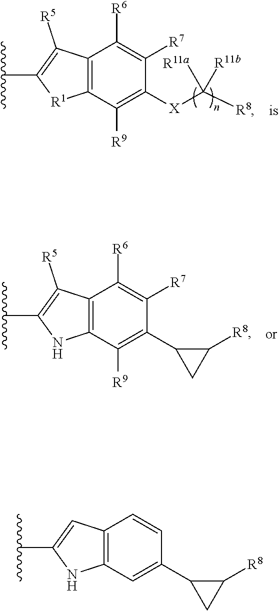

wherein R⁸ is C₆₋₂₀aryl. In some embodiments of a compound of formula (I), or a stereoisomer or tautomer thereof, or a pharmaceutically acceptable salt of any of the foregoing, the moiety represented by

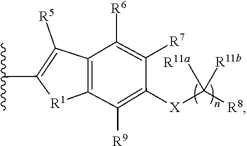

is

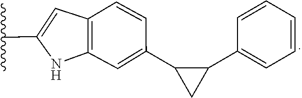 [SMILES: *c1cc2ccc(C3CC3c3ccccc3)cc2[nH]1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, the moiety represented by

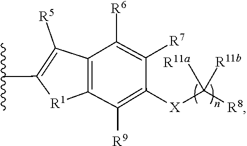

is

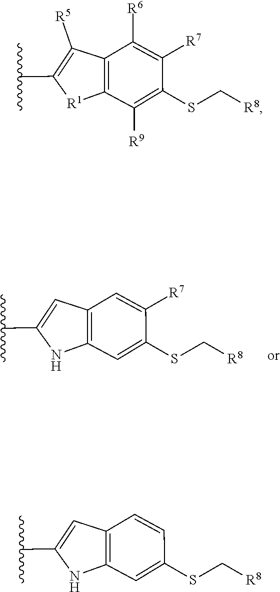

wherein R⁸ is 5-20 membered heteroaryl. In some embodiments of a compound of formula (I), or a stereoisomer or tautomer thereof, or a pharmaceutically acceptable salt of any of the foregoing, the moiety represented by

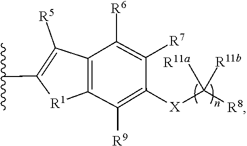

is selected from the group consisting of

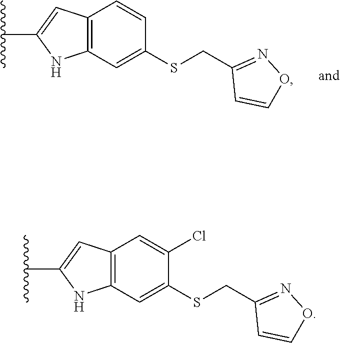 [SMILES: *c1cc2ccc(SCc3ccon3)cc2[nH]1 ; *c1cc2cc(Cl)c(SCc3ccon3)cc2[nH]1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or a stereoisomer or tautomer thereof, or a pharmaceutically acceptable salt of any of the foregoing, R¹ is —NH—, —O—, or —S—. In some embodiments, R¹ is —O—. In some embodiments, R¹ is —S—. In some embodiments, R¹ is —NH—. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), such as a compound of formula (I-A):

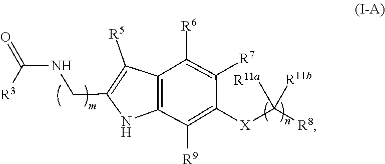

or a stereoisomer or tautomer thereof, or a pharmaceutically acceptable salt of any of the foregoing, wherein m, n, R³, R⁵, R⁶, R⁷, R⁸, R⁹, R^{11a}, R^{11b}, and X are as defined for a compound of formula (I). In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, m is an integer from 1-4. In some embodiments, m is an integer from 1-2. In some embodiments, m is 1. In some embodiments, m is 2. In some embodiments, m is 3. In some embodiments, m is 4. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, n is an integer from 0-4. In some embodiments, n is an integer from 0-2. In some embodiments, n is 0. In some embodiments, n is 1. In some embodiments, n is 2. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, m is an integer from 1-4, and n is an integer from 0-4. In some embodiments, m is an integer from 1-2, and n is an integer from 0-2. In some embodiments, m is 1, and n is 0. In some embodiments, m is 1, and n is 1. In some embodiments, m is 1, and n is 2. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, X is —O—, —S—, —C(R^{12a})(R^{12b})—, —N(H)—, or —N(C₁₋₆alkyl)-. In some embodiments, X is —O—, —S—, —C(R^{12a})(R^{12b})—, —N(H)—, or —N(C₁₋₃alkyl)-. In some embodiments, X is —O—. In some embodiments, X is —S—. In some embodiments, X is —C(R^{12a})(R^{12b})—. In some embodiments, X is —N(H)—. In some embodiments, X is —N(C₁₋₃alkyl)-. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, X is —CH₂—, —CH(C₁₋₆alkyl)-, or —C(C₁₋₆alkyl)₂-. In some embodiments, X is —CH₂—, —CH(C₁₋₃alkyl)-, or —C(C₁₋₃alkyl)₂-. In some embodiments, X is —CH₂—. In some embodiments, X is —CH(CH₃)—. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, X is —N(R¹²)—. In some embodiments, X is —NH—. In some embodiments, X is —NMe-. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}. In some embodiments, R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}. In some embodiments, R³ is C₁₋₃alkyl, C₁₋₃haloalkyl, C₃₋₆cycloalkyl, —N(R⁴)₂, or 4-6 membered heterocyclyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R³ is C₁₋₆alkyl, optionally substituted with one or more R^{a}. In some embodiments, R³ is C₁₋₃alkyl. In some embodiments, R³ is methyl, ethyl, or isopropyl. In some embodiments, R³ is methyl. In some embodiments, R³ is ethyl.

In some embodiments, R³ is isopropyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof. In some embodiments, R³ is selected from the group consisting of

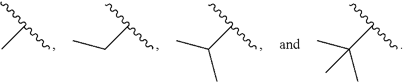

In some embodiments of a compound of formula (I′), or (I-A), or a stereoisomer or tautomer thereof, or a pharmaceutically acceptable salt of any of the foregoing, R³ is C₁₋₆alkyl, optionally substituted with one or more R^{a}; R^{a} is —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, 4-10 membered heterocyclyl, or C₃₋₁₀cycloalkyl, wherein the C₁₋₆ alkyl, C₁₋₆ alkoxy, 4-10 membered heterocyclyl or C₃₋₁₀cycloalkyl of R^{a} is optionally substituted with one or more R^{b}; and each R^{b} is, independently at each occurrence, —OH, halo, C₁₋₆alkyl or C₁₋₆alkoxy. In some embodiments, R³ is C₁₋₃alkyl, optionally substituted with one or more R^{a}; R^{a} is —OH, oxo, —CN, halo, C₁₋₃alkyl, C₁₋₃alkoxy, C₁₋₃haloalkyl, —O—C₁₋₃haloalkyl, —NH₂, —NH(C₁₋₃alkyl), —N(C₁₋₃alkyl)₂, 4-6 membered heterocyclyl, or C₃₋₆cycloalkyl, wherein the C₁₋₃alkyl, C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy. In some embodiments, R³ is C₁₋₃alkyl, optionally substituted with one or more R^{a}; R^{a} is —OH, halo, C₁₋₃alkoxy, 4-6 membered heterocyclyl, or C₃₋₆cycloalkyl, wherein the C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy. In some embodiments, R³ is selected from the group consisting of

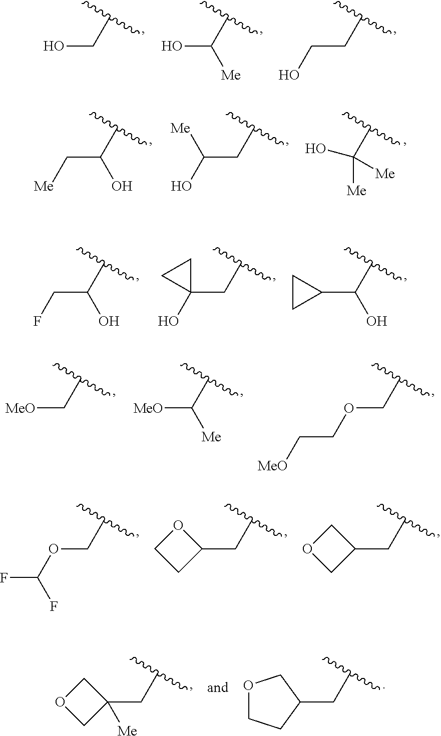

In some embodiments of a compound of formula (II), or a stereoisomer or tautomer thereof, or a pharmaceutically acceptable salt of any of the foregoing, R³ is C₁₋₆alkyl, optionally substituted with one or more R^{a}; R^{a} is D, —OH, oxo, —CN, halo, C₁₋₆ alkyl, C₁₋₆alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, 5-20 membered heteroaryl, C₃₋₁₀cycloalkyl, or —O—C₃₋₁₀cycloalkyl, wherein the C₁₋₆ alkyl, C₁₋₆alkoxy, 4-10 membered heterocyclyl, C₃₋₁₀cycloalkyl, or —O—C₃₋₁₀cycloalkyl of R^{a} is optionally substituted with one or more R^{b}; and each R^{b} is independently D, —OH, halo, C₁₋₆ alkyl, or C₁₋₆ alkoxy. In some embodiments, R³ is C₁₋₃alkyl, optionally substituted with one or more R^{a}; R^{a} is D, —OH, oxo, —CN, halo, C₁₋₃alkyl, C₁₋₃alkoxy, C₁₋₃haloalkyl, —O—C₁₋₃haloalkyl, —NH₂, —NH(C₁₋₃alkyl), —N(C₁₋₃alkyl)₂, —C(O)NH₂, —C(O)NH(C₁₋₃alkyl), —C(O)N(C₁₋₃alkyl)₂, 4-6 membered heterocyclyl, 5-10 membered heteroaryl, C₃₋₆cycloalkyl, or —O—C₃₋₆cycloalkyl, wherein the C₁₋₃alkyl, C₁₋₃alkoxy, 4-10 membered heterocyclyl, C₃₋₁₀cycloalkyl, or —O—C₃₋₁₀cycloalkyl of R^{a} is optionally substituted with one or more R^{b}; and each R^{b} is independently D, —OH, halo, C₁₋₃alkyl, or C₁₋₃alkoxy. In some embodiments, R³ is C₁₋₃alkyl, optionally substituted with one or more R^{a}; R^{a} is —OH, halo, C₁₋₃alkoxy, 4-6 membered heterocyclyl, or C₃₋₆cycloalkyl, wherein the C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy. In some embodiments, R³ is selected from the group consisting of

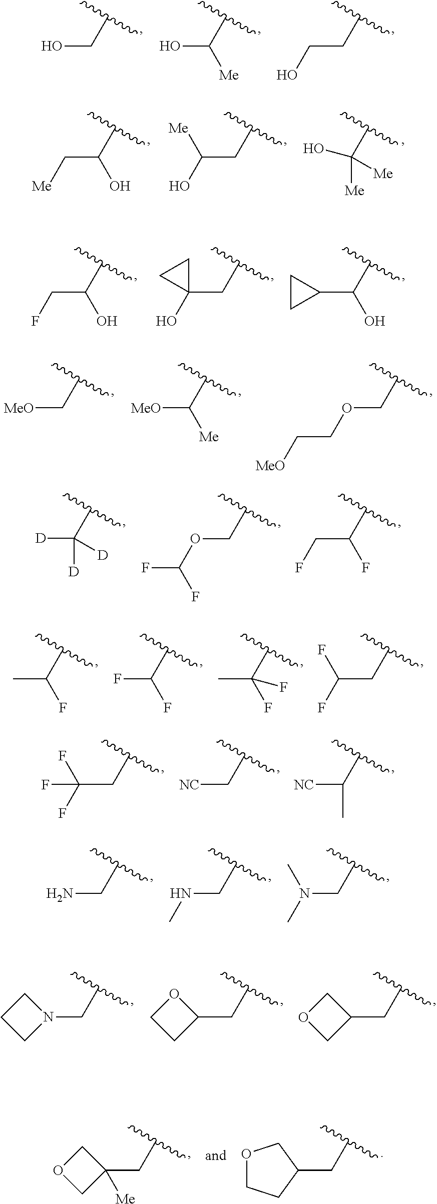

In some embodiments of a compound of formula (II), or a stereoisomer or tautomer thereof, or a pharmaceutically acceptable salt of any of the foregoing, R³ is C₁₋₆alkoxy optionally substituted with one or more R^{a}. In some embodiments, R³ is C₁₋₃alkoxy. In some embodiments, R³ is methoxy, ethoxy, or isopropoxy. In some embodiments, R³ is methoxy. In some embodiments, R³ is ethoxy. In some embodiments, R³ is isopropoxy.

In some embodiments of a compound of formula (II), or a stereoisomer or tautomer thereof, or a pharmaceutically acceptable salt of any of the foregoing, R³ is —C(O)C₁₋₆alkyl optionally substituted with one or more R^{a}. In some embodiments, R³ is —C(O)C₁₋₃alkyl. In some embodiments, R³ is —C(O)methyl, or —C(O)methylethyl. In some embodiments, R³ is —C(O)methyl.

In some embodiments of a compound of formula (I), or (I-A), or a stereoisomer or tautomer thereof, or a pharmaceutically acceptable salt of any of the foregoing, R³ is C₃₋₁₀cycloalkyl optionally substituted with one or more R^{a}, and R^{a} is, —OH, halo, C₁₋₆alkyl, C₁₋₆haloalkyl, or C₃₋₁₀cycloalkyl. In some embodiments, R³ is C₃₋₆cycloalkyl optionally substituted with one or more R^{a}, and R^{a} is, —OH, halo, C₁₋₃alkyl, C₁₋₃haloalkyl, or C₃₋₆cycloalkyl. In some embodiments, R³ is C₃₋₆cycloalkyl optionally substituted with one or more R^{a}, and R^{a} is C₁₋₃alkyl, or C₁₋₃haloalkyl. In some embodiments, R³ is C₃₋₆cycloalkyl optionally substituted with one or more C₁₋₃alkyl, or C₁₋₃ haloalkyl. In some embodiments, R³ is cyclopropyl, optionally substituted with one or more C₁₋₃alkyl, or C₁₋₃haloalkyl. In some embodiments, R³ is selected from the group consisting of

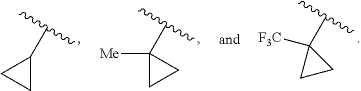

In some embodiments, R³ is C₃₋₆cycloalkyl optionally substituted with one or more R^{a}, and R^{a} is halo. In some embodiments, R³ is C₃₋₆cycloalkyl optionally substituted with one or more Br, Cl, I, or F. In some embodiments, R³ is cyclopropyl, optionally substituted with one or more Br, Cl, I, or F. In some embodiments, R³ is

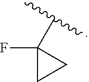 [SMILES: *C1(F)CC1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R³ is C₃₋₁₀cycloalkyl optionally substituted with one or more R^{a}; R^{a} is, independently at each occurrence, —OH, oxo, —CN, halo, C₁₋₆alkyl, C₁₋₆ alkoxy, C₁₋₆ haloalkyl, —O—C₁₋₆haloalkyl, —NH₂, —NH(C₁₋₆alkyl), —N(C₁₋₆ alkyl)₂, 4-10 membered heterocyclyl, or C₃₋₁₀cycloalkyl, wherein the C₁₋₆alkyl, C₁₋₆alkoxy, 4-10 membered heterocyclyl or C₃₋₁₀cycloalkyl of R^{a} is optionally substituted with one or more R^{b}; and each R^{b} is, independently at each occurrence, —OH, halo, C₁₋₆alkyl or C₁₋₆alkoxy. In some embodiments, R³ is C₃₋₆cycloalkyl optionally substituted with one or more R^{a}; R^{a} is, independently at each occurrence, —OH, oxo, —CN, halo, C₁₋₃alkyl, C₁₋₃alkoxy, C₁₋₃haloalkyl, —O—C₁₋₃haloalkyl, —NH₂, —NH(C₁₋₃alkyl), —N(C₁₋₃alkyl)₂, 4-6 membered heterocyclyl, or C₃₋₆cycloalkyl, wherein the C₁₋₃alkyl, C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy. In some embodiments, R³ is C₃₋₆cycloalkyl optionally substituted with one or more R^{a}; R^{a} is, independently at each occurrence, —OH, halo, C₁₋₃alkyl, C₁₋₃haloalkyl, wherein the C₁₋₃alkyl of R^{a} is optionally substituted with one or more R^{b}; and each R^{b} is, independently at each occurrence, —OH, or C₁₋₃alkoxy. In some embodiments, R³ is C₃₋₆cycloalkyl optionally substituted with one or more —OH, halo, C₁₋₃alkyl, C₁₋₃haloalkyl, wherein the C₁₋₃alkyl is optionally substituted with one or more —OH, or C₁₋₃alkoxy. In some embodiments, R³ is selected from the group consisting of

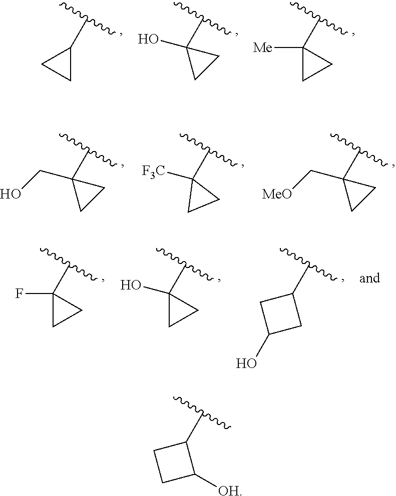

In some embodiments of a compound of formula (II), or a stereoisomer or tautomer thereof, or a pharmaceutically acceptable salt of any of the foregoing, R³ is C₃₋₆cycloalkyl optionally substituted with one or more —OH, halo, C₁₋₃alkyl, or C₁₋₃haloalkyl, wherein the C₁₋₃alkyl is optionally substituted with one or more D, —OH, or C₁₋₃alkoxy. In some embodiments, R³ is selected from the group consisting of

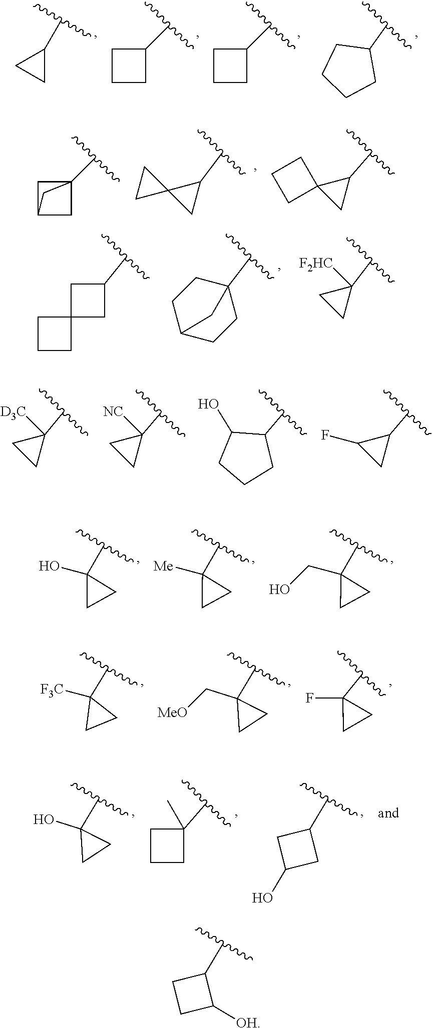

In some embodiments of a compound of formula (I), or (I-A), or a stereoisomer or tautomer thereof, or a pharmaceutically acceptable salt of any of the foregoing, R³ is —N(R⁴)₂, and each R⁴ is independently H, C₁₋₆alkyl, C₁₋₆haloalkyl, or C₃₋₁₀cycloalkyl. In some embodiments, R³ is —N(R⁴)₂, and each R⁴ is independently H, C₁₋₃alkyl, C₁₋₃haloalkyl, or C₃₋₆cycloalkyl. In some embodiments, R³ is —N(R⁴)₂, and each R⁴ is independently H, or C₁₋₃alkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R³ is 4-10 membered heterocyclyl optionally substituted with one or more R^{a}, and R^{a} is, —OH, halo, C₁₋₆alkyl, C₁₋₆haloalkyl, or C₃₋₁₀cycloalkyl. In some embodiments, R³ is 4-6 membered heterocyclyl, optionally substituted with one or more R^{a}, and R^{a} is, —OH, halo, C₁₋₃alkyl, C₁₋₃haloalkyl, or C₃₋₆cycloalkyl. In some embodiments, R³ is 4-6 membered heterocyclyl, optionally substituted with one or more R^{a}, R^{a} is halo, C₁₋₃alkyl, or C₁₋₃haloalkyl. In some embodiments, 4-6 membered heterocyclyl optionally substituted with one or more halo, C₁₋₃alkyl, or C₁₋₃haloalkyl. In some embodiments, R³ is azetidine, pyrrolidine, or piperidine, wherein each R³ is optionally substituted with one or more halo, C₁₋₃alkyl, or C₁₋₃haloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), or (I-A), or a stereoisomer or tautomer thereof, or a pharmaceutically acceptable salt of any of the foregoing, R³ is 4-10 membered heterocyclyl optionally substituted with one or more R^{a}; R^{a} is, independently at each occurrence, —OH, oxo, —CN, halo, C₁₋₆ alkyl, C₁₋₆ alkoxy, C₁₋₆ haloalkyl, —O—C₁₋₆ haloalkyl, —NH₂, —NH(C₁₋₆alkyl), —N(C₁₋₆ alkyl)₂, 4-10 membered heterocyclyl, or C₃₋₁₀cycloalkyl, wherein the C₁₋₆ alkyl, C₁₋₆alkoxy, 4-10 membered heterocyclyl or C₃₋₁₀cycloalkyl of R^{a} is optionally substituted with one or more R^{b}, and each R^{b} is, independently at each occurrence, —OH, halo, C₁₋₆alkyl or C₁₋₆alkoxy. In some embodiments, R³ is 4-6 membered heterocyclyl, optionally substituted with one or more R^{a}, and R^{a} is, —OH, halo, C₁₋₃alkyl, C₁₋₃haloalkyl, or C₃₋₆cycloalkyl. In some embodiments, R³ is 4-6 membered heterocyclyl, optionally substituted with one or more R^{a}; R^{a} is, independently at each occurrence, —OH, oxo, —CN, halo, C₁₋₃alkyl, C₁₋₃alkoxy, C₁₋₃haloalkyl, —O—C₁₋₃haloalkyl, —NH₂, —NH(C₁₋₃alkyl), —N(C₁₋₃alkyl)₂, 4-6 membered heterocyclyl, or C₃₋₆cycloalkyl, wherein the C₁₋₃alkyl, C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy. In some embodiments, R³ is 4-6 membered heterocyclyl optionally substituted with one or more R^{a}; R^{a} is, independently at each occurrence, —OH, halo, C₁₋₃alkyl, C₁₋₃haloalkyl, wherein the C₁₋₃alkyl of R^{a} is optionally substituted with one or more R^{b}; and each R^{b} is, independently at each occurrence, —OH, or C₁₋₃alkoxy. In some embodiments, R³ is 4-6 membered heterocyclyl optionally substituted with one or more R^{a}; R^{a} is, independently at each occurrence, —CN, halo, or C₁₋₃alkyl, wherein the C₁₋₃alkyl of R^{a} is optionally substituted with one or more R^{b}; and each R^{b} is OH. In some embodiments, R³ is 4-6 membered heterocyclyl optionally substituted with one or more —CN, halo, or C₁₋₃alkyl, wherein the C₁₋₃alkyl of R^{a} is optionally substituted with one or more OH. In some embodiments, R³ is selected from the group consisting of

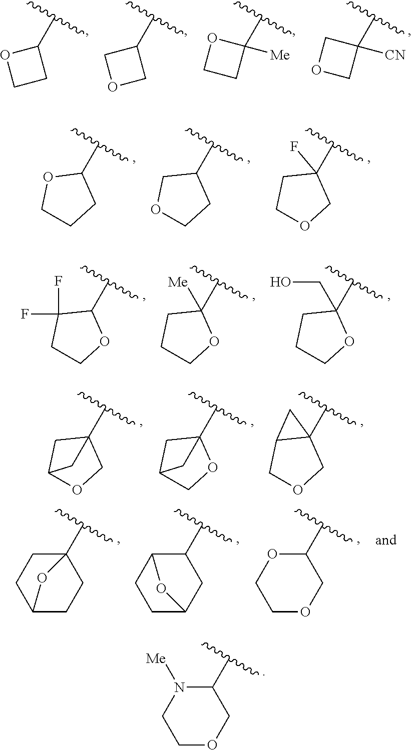

In some embodiments, R³ is selected from the group consisting of

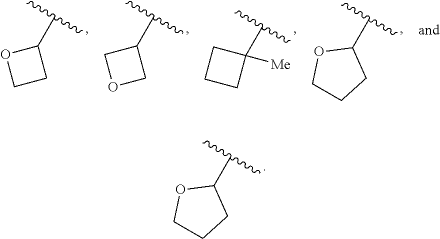

In some embodiments of a compound of formula (I), or (I-A), or a stereoisomer or tautomer thereof, or a pharmaceutically acceptable salt of any of the foregoing, R³ is —N(R⁴)₂, and both R⁴ are taken together with the N atom to which they are attached to form a 4-10 membered heterocyclyl optionally substituted with one or more R^{a}, and R^{a} is OH, halo, C₁₋₆alkyl, C₁₋₆haloalkyl, or C₃₋₁₀cycloalkyl. In some embodiments, R³ is —N(R⁴)₂, and both R⁴ are taken together with the N atom to which they are attached to form a 4-6 membered heterocyclyl optionally substituted with one or more R^{a}, and R^{a} is OH, halo, C₁₋₆alkyl, C₁₋₆haloalkyl, or C₃₋₁₀cycloalkyl. In some embodiments, R³ is —N(R⁴)₂, and both R⁴ are taken together with the N atom to which they are attached to form a 4-6 membered heterocyclyl optionally substituted with one or more R^{a}, and R^{a} is halo, C₁₋₃alkyl, or C₁₋₃haloalkyl. In some embodiments, R³ is —N(R⁴)₂, and both R⁴ are taken together with the N atom to which they are attached to form a 4-6 membered heterocyclyl optionally substituted with one or more halo, C₁₋₃alkyl, or C₁₋₃haloalkyl. In some embodiments, R³ is selected from the group consisting of

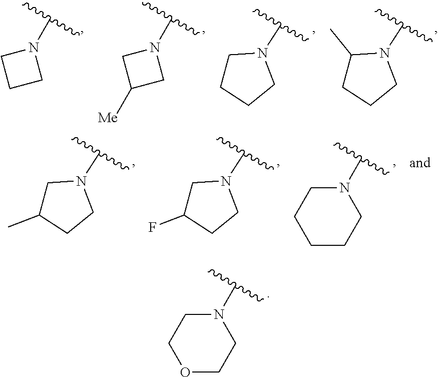

In some embodiments, R³ is selected from the group consisting of

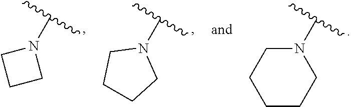 [SMILES: *N1CCC1 ; *N1CCCC1 ; *N1CCCCC1]

In some embodiments, R³ is

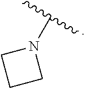 [SMILES: *N1CCC1]

In some embodiments, R³ is

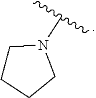 [SMILES: *N1CCCC1]

In some embodiments, R³ is

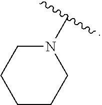 [SMILES: *N1CCCCC1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, R³ is —N(R⁴)₂, and both R⁴ are taken together with the N atom to which they are attached to form a 4-10 membered heterocyclyl optionally substituted with one or more R^{a}; R^{a} is OH, CN, halo, C₁₋₆alkyl, C₁₋₆ haloalkyl, C₁₋₆alkoxy, or C₃₋₁₀cycloalkyl; and the C₁₋₆ alkyl or R^{a} is optionally substituted with one or more OH. In some embodiments, R³ is selected from the group consisting of

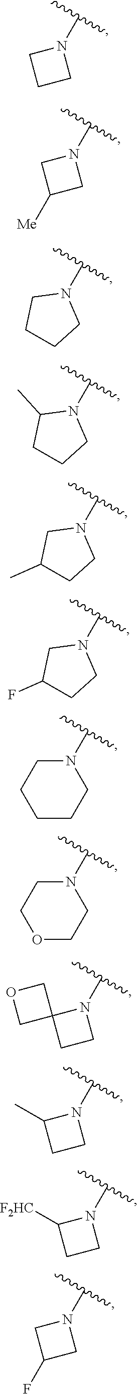

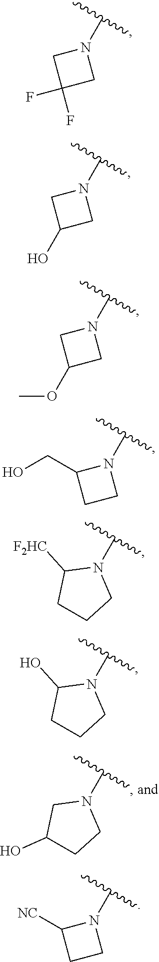

In some embodiments of a compound of formula (II), or a stereoisomer or tautomer thereof, or a pharmaceutically acceptable salt of any of the foregoing, R³ is 5-20 membered heteroaryl optionally substituted with one or more R^{a}, wherein when R³ is 6-10 membered heteroaryl, the 6-10 membered heteroaryl is unsubstituted. In some embodiments, R³ is 5 membered heteroaryl optionally substituted with one or more R^{a}. In some embodiments, R³ is 5 membered heteroaryl. In some embodiments, R³ is

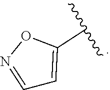 [SMILES: *c1ccno1]

In some embodiments of a compound of formula (II), or a stereoisomer or tautomer thereof, or a pharmaceutically acceptable salt of any of the foregoing, R³ is —C₁₋₆alkyl(5-20 membered heteroaryl), optionally substituted with one or more R^{a}. In some embodiments, R³ is —C₁₋₆alkyl(5 membered heteroaryl), optionally substituted with one or more R^{a}. In some embodiments, R³ is —C₁₋₆alkyl(5 membered heteroaryl). In some embodiments, R³ is

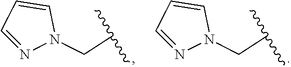

In some embodiments of a compound of formula (II), or a stereoisomer or tautomer thereof, or a pharmaceutically acceptable salt of any of the foregoing, R⁵ and R⁷ are each independently H, halo, C₁₋₆alkyl, C₁₋₆alkoxy, C₁₋₆thioalkyl, C₃₋₁₀cycloalkyl, C₁₋₆haloalkyl, or 5-20 membered heteroaryl, wherein the C₁₋₆alkyl, C₁₋₆alkoxy, or C₃₋₁₀cycloalkyl of R⁵ and R⁷ are each independently optionally substituted with one or more halo or CN. In some embodiments, R⁵ and R⁷ are each independently H, halo, C₁₋₃alkyl, C₁₋₃alkoxy, C₁₋₃thioalkyl, C₃₋₆cycloalkyl, C₁₋₃haloalkyl, or 5-10 membered heteroaryl, wherein the C₁₋₃alkyl, C₁₋₃alkoxy, or C₃₋₆cycloalkyl of R⁵ and R⁷ are each independently optionally substituted with one or more halo or CN.

In some embodiments of a compound of formula (I), or (I-A), or a stereoisomer or tautomer thereof, or a pharmaceutically acceptable salt of any of the foregoing, R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN. In some embodiments, R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN. In some embodiments, R⁵ and R⁷ are each independently H, halo, C₁₋₃alkyl, or C₁₋₃haloalkyl. In some embodiments, R⁵ and R⁷ are each independently H. In some embodiments, one of R⁵ and R⁷ is H, and the other of R⁵ and R⁷ is independently halo, C₁₋₃alkyl, or C₁₋₃haloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R⁶ and R⁹ are each independently H, or halo. In some embodiments, R⁶ and R⁹ are each independently H, F, or Cl. In some embodiments, R⁶ and R⁹ are each independently H. In some embodiments, R⁶ and R⁹ are each independently halo. In some embodiments, R⁶ and R⁹ are each independently F, or Cl. In some embodiments, one of R⁶ and R⁹ is H, and the other of R⁶ and R⁹ is halo. In some embodiments, one of R⁶ and R⁹ is H, and the other of R⁶ and R⁹ is F, or Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R⁸ is —C(O)N(R⁴)₂, C₃₋₁₀-cycloalkyl, C₆₋₂₀aryl, 3-10 membered heterocyclyl, or 5-20 membered heteroaryl, wherein each R⁸ is optionally substituted with one or more R^{a}, and R^{a} is OH, halo, C₁₋₆alkyl, C₁₋₆haloalkyl, or C₃₋₁₀cycloalkyl. In some embodiments, R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}, and R^{a} is OH, halo, C₁₋₃alkyl, C₁₋₃haloalkyl, or C₃₋₆cycloalkyl. In some embodiments, R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more OH, halo, C₁₋₃alkyl, C₁₋₃haloalkyl, or C₃₋₆cycloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R⁸ is —C(O)N(R⁴)₂. In some embodiments, R⁸ is —C(O)N(R⁴)₂, and each R⁴ is independently H, C₁₋₆alkyl, C₁₋₆haloalkyl, or C₃₋₁₀cycloalkyl. In some embodiments, R⁸ is —C(O)N(R⁴)₂, and each R⁴ is independently H, or C₁₋₆alkyl. In some embodiments, R⁸ is —C(O)NH₂, C(O)NHMe, or C(O)NMe₂. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, R⁸ is C₁₋₆alkoxy optionally substituted with one or more R^{a}. In some embodiments, R⁸ is C₁₋₆alkoxy, optionally substituted with one or more R^{a}, and R^{a} is halo. In some embodiments, R⁸ is C₁₋₃alkoxy. In some embodiments, R⁸ is C₁₋₃alkoxy, optionally substituted with one or more R^{a}, and R^{a} is halo. In some embodiments, R⁸ is selected from the group consisting of

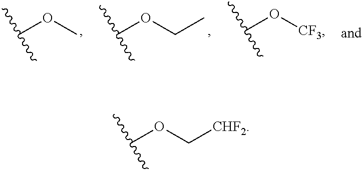

In some embodiments of a compound of formula (I), or (I-A), or a stereoisomer or tautomer thereof, or a pharmaceutically acceptable salt of any of the foregoing, R⁸ is C₃₋₁₀cycloalkyl. In some embodiments, R⁸ is C₃₋₁₀cycloalkyl, optionally substituted with one or more R^{a}, and R^{a} is OH, halo, C₁₋₆alkyl, C₁₋₆haloalkyl, or C₃₋₁₀cycloalkyl. In some embodiments, R⁸ is C₃₋₆cycloalkyl. In some embodiments, R⁸ is C₃₋₆cycloalkyl, optionally substituted with one or more R^{a}, and R^{a} is OH, halo, C₁₋₆alkyl, C₁₋₆haloalkyl, or C₃₋₁₀cycloalkyl. In some embodiments, R⁸ is selected from the group consisting of

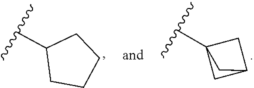 [SMILES: *C1CCCC1 ; *C12CC(C1)C2]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (II), or a stereoisomer or tautomer thereof, or a pharmaceutically acceptable salt of any of the foregoing, R⁸ is C₃₋₁₀cycloalkyl. In some embodiments, R⁸ is C₃₋₁₀cycloalkyl, optionally substituted with one or more R^{a}, and R^{a} is OH, halo, C₁₋₆alkyl, C₁₋₆haloalkyl, C₁₋₆alkoxy, or C₃₋₁₀cycloalkyl. In some embodiments, R⁸ is C₃₋₆cycloalkyl. In some embodiments, R⁸ is C₃₋₆cycloalkyl, optionally substituted with one or more R^{a}, and R^{a} is OH, halo, C₁₋₆alkyl, C₁₋₆haloalkyl, C₁₋₃alkoxy, or C₃₋₁₀cycloalkyl. In some embodiments, R⁸ is selected from the group consisting of

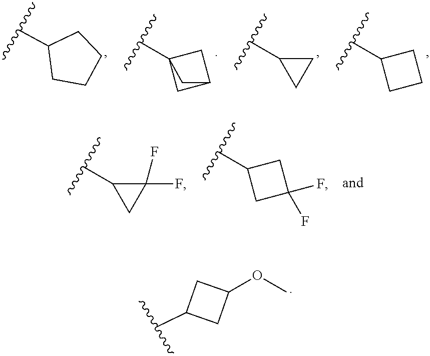

In some embodiments of a compound of formula (II), or a stereoisomer or tautomer thereof, or a pharmaceutically acceptable salt of any of the foregoing, R⁸ is —O—C₃₋₁₀cycloalkyl. In some embodiments, R⁸ is —O—C₃₋₁₀cycloalkyl, optionally substituted with one or more R^{a}. In some embodiments, R⁸ is —O—C₃₋₆cycloalkyl. In some embodiments, R⁸ is selected from the group consisting of

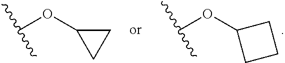 [SMILES: *OC1CC1 ; *OC1CCC1]

In some embodiments of a compound of formula (I), or (I-A), or a stereoisomer or tautomer thereof, or a pharmaceutically acceptable salt of any of the foregoing, R⁸ is C₆₋₁₂aryl. In some embodiments, R⁸ is C₆₋₁₂aryl, optionally substituted with one or more R^{a}, and R^{a} is OH, halo, C₁₋₆alkyl, C₁₋₆haloalkyl, or C₃₋₁₀cycloalkyl. In some embodiments, R⁸ is C₆₋₁₂aryl, optionally substituted with one or more —OH. In some embodiments, R^{a} is selected from the group consisting of

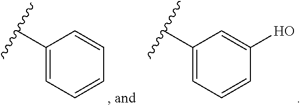 [SMILES: *c1ccccc1 ; *c1cccc(O)c1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R⁸ is 3-10 membered heterocyclyl. In some embodiments, R⁸ is 3-10 membered heterocyclyl, optionally substituted with one or more R^{a}, and R^{a} is OH, halo, C₁₋₆ alkyl, C₁₋₆ haloalkyl, or C₃₋₁₀cycloalkyl. In some embodiments, R⁸ is 3-6 membered heterocyclyl. In some embodiments, R⁸ is 3-6 membered heterocyclyl, optionally substituted with one or more R^{a}, and R^{a} is OH, halo, C₁₋₆alkyl, C₁₋₆haloalkyl, or C₃₋₁₀cycloalkyl. In some embodiments, R⁸ is

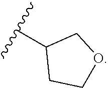 [SMILES: *C1CCOC1]

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof. In some embodiments, R⁸ is selected from the group consisting of

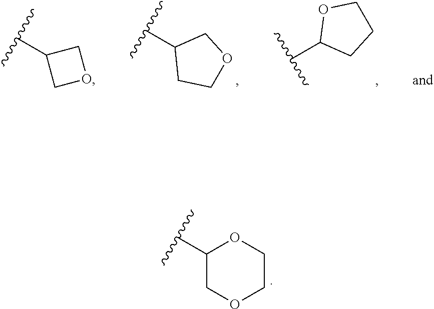 [SMILES: *C1COC1 ; *C1CCOC1 ; *C1CCCO1 ; *C1COCCO1]

In some embodiments of a compound of formula (I), or (I-A), or a stereoisomer or tautomer thereof, or a pharmaceutically acceptable salt of any of the foregoing, R⁸ is 5-20 membered heteroaryl. In some embodiments, R⁸ is 5-20 membered heteroaryl, optionally substituted with one or more R^{a}, and R^{a} is OH, halo, C₁₋₆ alkyl, C₁₋₆ haloalkyl, or C₃₋₁₀cycloalkyl. In some embodiments, R⁸ is 5-10 membered heteroaryl, optionally substituted with one or more halo, C₁₋₆ alkyl, C₁₋₆haloalkyl, or C₃₋₁₀cycloalkyl. In some embodiments, R⁸ is selected from the group consisting of

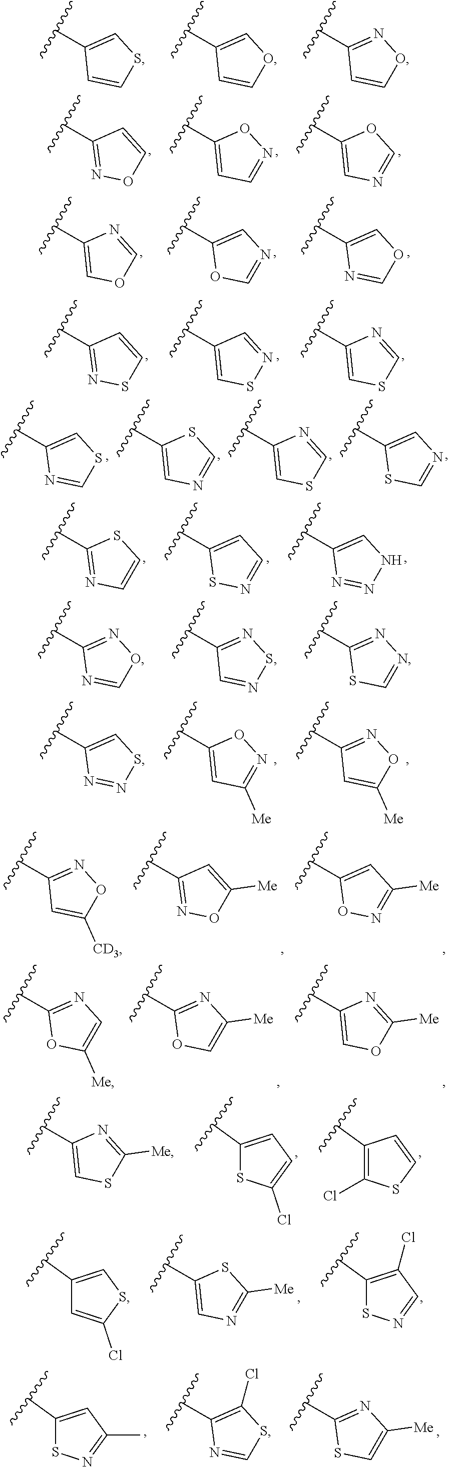

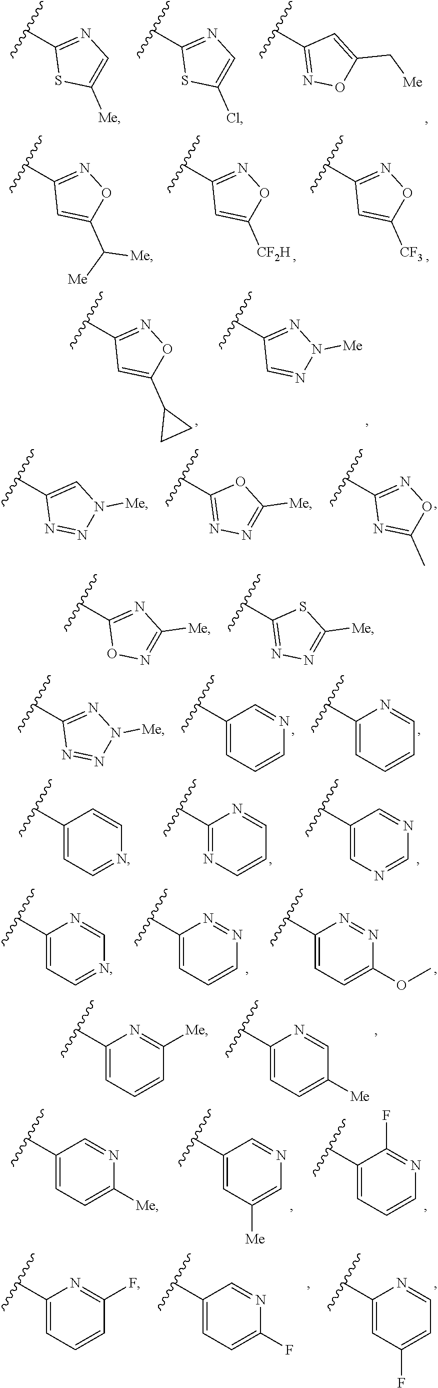

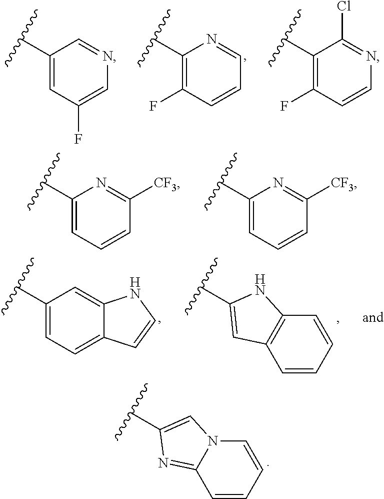

In some embodiments, R⁸ is selected from the group consisting of

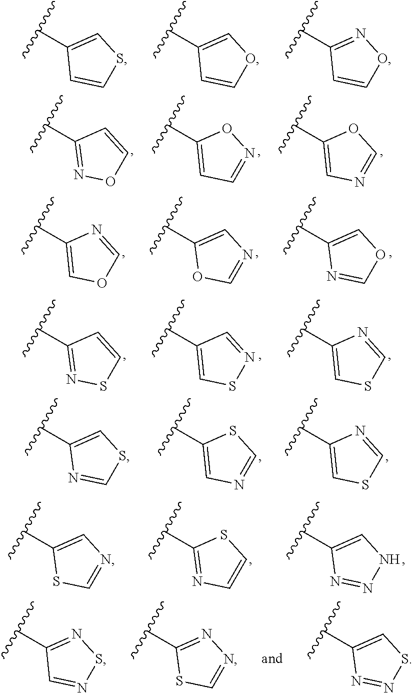

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, R^{11a} and R^{11b} are each, independently at each occurrence, H, or C₁₋₆alkyl. In some embodiments, R^{11a} and R^{11b} are each, independently at each occurrence, H, or C₁₋₃alkyl. In some embodiments, R^{11a} and R^{11b} are each, independently at each occurrence, H. In some embodiments, one of R^{11a} and R^{11b} is H, and the other of R^{11a} and R^{11b} is C₁₋₃alkyl. In some embodiments, one of R^{11a} and R^{11b} is H, and the other of R^{11a} and R^{11b} is methyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), or a stereoisomer or tautomer thereof, or a pharmaceutically acceptable salt of any of the foregoing, n is 1; one of R^{11a} and R^{11b} is H, or C₁₋₆alkyl; and the other of R^{11a} and R^{11b} is taken together with one of R^{12a} or R^{12b} to form a C₃₋₁₀cycloalkyl. In some embodiments, n is 1; one of R^{11a} and R^{11b} is H, or C₁₋₃alkyl; and the other of R^{11a} and R^{11b} is taken together with one of R^{12a} or R^{12b} to form a C₃₋₆cycloalkyl. In some embodiments, n is 1; one of R^{11a} and R^{11b} is H, or C₁₋₆alkyl; and the other of R^{11a} and R^{11b} is taken together with one of R^{12a} or R^{12b} to form cyclopropyl. In some embodiments, n is 1; one of R^{11a} and R^{11b} is H; and the other of R^{11a} and R^{11b} is taken together with one of R^{12a} or R^{12b} to form cyclopropyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-A1):

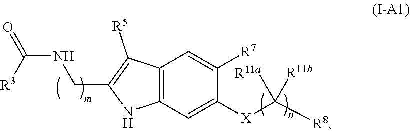

or a stereoisomer or tautomer thereof, or a pharmaceutically acceptable salt of any of the foregoing, wherein m, n, R³, R⁵, R⁷, R⁸, R^{11a}, R^{11b}, and X are as defined for a compound of formula (I). In some variations, m, n, R³, R⁵, R⁷, R⁸, R^{11a}, R^{11b}, and X of formula (I-A1) are as defined for a compound of formula (I′), or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ and R⁷ are each independently H, halo, C₁₋₆alkyl, C₃₋₁₀cycloalkyl or C₁₋₆haloalkyl, wherein the C₁₋₆alkyl of R⁵ and R⁷ are each independently optionally substituted with one or more halo, or CN. In some embodiments, R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN. In some embodiments, R⁵ and R⁷ are each independently H, halo, C₁₋₃alkyl, or C₁₋₃haloalkyl. In some embodiments, R⁵ and R⁷ are each independently H. In some embodiments, one of R⁵ and R⁷ is H, and the other of R⁵ and R⁷ is independently halo, C₁₋₃alkyl, or C₁₋₃haloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ and R⁷ are each H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ is H, and R⁷ is halo. In some embodiments, R⁵ is H, and R⁷ is F. In some embodiments, R⁵ is H, and R⁷ is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ is H, and R⁷ is C₁₋₆alkyl. In some embodiments, R⁵ is H, and R⁷ is C₁₋₃alkyl. In some embodiments, R⁵ is H, and R⁷ is methyl. In some embodiments, R⁵ is H, and R⁷ is ethyl. In some embodiments, R⁵ is H, and R⁷ is isopropyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ is H, and R⁷ is C₁₋₆haloalkyl. In some embodiments, R⁵ is H, and R⁷ is C₁₋₃haloalkyl. In some embodiments, R⁵ is H, and R⁷ is CF₃. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ is halo, and R⁷ is H. In some embodiments, R⁵ is F, and R⁷ is H. In some embodiments, R⁵ is Cl, and R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A1), or a stereoisomer or tautomer thereof, or a pharmaceutically acceptable salt of any of the foregoing, R⁵ is C₁₋₆alkyl, and R⁷ is H. In some embodiments, R⁵ is C₁₋₃alkyl, and R⁷ is H. In some embodiments, R⁵ is methyl, and R⁷ is H. In some embodiments, R⁵ is ethyl, and R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-A2):

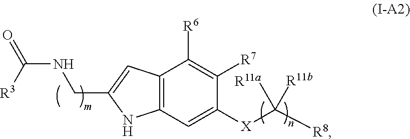

or a stereoisomer or tautomer thereof, or a pharmaceutically acceptable salt of any of the foregoing, wherein m, n, R³, R⁶, R⁷, R⁸, R^{11a}, R^{11b}, and X are as defined for a compound of formula (I). In some variations, m, n, R³, R⁶, R⁷, R⁸, R^{11a}, R^{11b}, and X of formula (I-A2) are as defined for a compound of formula (I′), or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is H, or halo; and R⁷ is H, halo, C₁₋₆alkyl, C₃₋₁₀cycloalkyl or C₁₋₆haloalkyl, wherein the C₁₋₆alkyl of R⁷ is optionally substituted with one or more halo, or CN. In some embodiments, R⁶ is H, or halo; and R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN. In some embodiments, R⁶ is H, or halo; and R⁷ is H, halo, C₁₋₃alkyl, or C₁₋₃haloalkyl. In some embodiments, R⁶ is H, or halo; and R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is H; and R⁷ is H, halo, C₁₋₆alkyl, C₃₋₁₀cycloalkyl or C₁₋₆haloalkyl, wherein the C₁₋₆alkyl of R⁷ is optionally substituted with one or more halo, or CN. In some embodiments, R⁶ is H; and R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN. In some embodiments, R⁶ is H; and R⁷ is H, halo, C₁₋₃alkyl, or C₁₋₃haloalkyl. In some embodiments, R⁶ is H; and R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is halo; and R⁷ is H, halo, C₁₋₆alkyl, C₃₋₁₀cycloalkyl or C₁₋₆haloalkyl, wherein the C₁₋₆alkyl of R⁷ is optionally substituted with one or more halo, or CN. In some embodiments, R⁶ is halo; and R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN. In some embodiments, R⁶ is halo; and R⁷ is H, halo, C₁₋₃alkyl, or C₁₋₃haloalkyl. In some embodiments, R⁶ is halo; and R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ and R⁷ are each H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is H, and R⁷ is halo. In some embodiments, R⁶ is H, and R⁷ is F. In some embodiments, R⁶ is H, and R⁷ is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is H, and R⁷ is C₁₋₆alkyl. In some embodiments, R⁶ is H, and R⁷ is C₁₋₃alkyl. In some embodiments, R⁶ is H, and R⁷ is methyl. In some embodiments, R⁶ is H, and R⁷ is ethyl. In some embodiments, R⁶ is H, and R⁷ is isopropyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is H, and R⁷ is C₁₋₆haloalkyl. In some embodiments, R⁶ is H, and R⁷ is C₁₋₃haloalkyl. In some embodiments, R⁶ is H, and R⁷ is CF₃. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ is halo, and R⁷ is H. In some embodiments, R⁶ is F, and R⁷ is H. In some embodiments, R⁶ is Cl, and R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A2), or a stereoisomer or tautomer thereof, or a pharmaceutically acceptable salt of any of the foregoing, R⁶ and R⁷ are each halo. In some embodiments, R⁶ is F and R⁷ is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-A3):

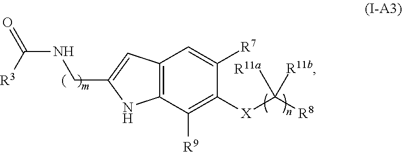

or a stereoisomer or tautomer thereof, or a pharmaceutically acceptable salt of any of the foregoing, wherein m, n, R³, R⁷, R⁸, R⁹, R^{11a}, R^{11b}, and X are as defined for a compound of formula (I). In some variations, m, n, R³, R⁷, R⁸, R⁹, R^{11a}, R^{11b}, and X of formula (I-A3) are as defined for a compound of formula (I′), or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is H, halo, C₁₋₆alkyl, C₃₋₁₀cycloalkyl or C₁₋₆haloalkyl, wherein the C₁₋₆alkyl of R⁷ is optionally substituted with one or more halo, or CN; and R⁹ is H, or halo. In some embodiments, R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN; and R⁹ is H, or halo. In some embodiments R⁷ is H, halo, C₁₋₃alkyl, or C₁₋₃haloalkyl; and R⁹ is H, or halo. In some embodiments, R⁷ is H; and R⁹ is H, or halo. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is H, halo, C₁₋₆alkyl, C₃₋₁₀cycloalkyl or C₁₋₆haloalkyl, wherein the C₁₋₆alkyl of R⁷ is optionally substituted with one or more halo, or CN; and R⁹ is H. In some embodiments, R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN; and R⁹ is H. In some embodiments, R⁷ is H, halo, C₁₋₃alkyl, or C₁₋₃haloalkyl; and R⁹ is H. In some embodiments, R⁷ is H; and R⁹ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is H, halo, C₁₋₆alkyl, C₃₋₁₀cycloalkyl or C₁₋₆haloalkyl, wherein the C₁₋₆alkyl of R⁷ is optionally substituted with one or more halo, or CN; and R⁹ is halo. In some embodiments, R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN; and R⁹ is halo. In some embodiments, R⁷ is H, halo, C₁₋₃alkyl, or C₁₋₃haloalkyl; and R⁹ is halo. In some embodiments, R⁷ is H; and R⁹ is halo. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ and R⁹ are each H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is halo; and R⁹ is H. In some embodiments, R⁷ is F; and R⁹ is H. In some embodiments, R⁷ is Cl; and R⁹ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is C₁₋₆alkyl; and R⁹ is H. In some embodiments, R⁷ is C₁₋₃alkyl; and R⁹ is H. In some embodiments, R⁷ is methyl; and R⁹ is H. In some embodiments, R⁷ is ethyl; and R⁹ is H. In some embodiments, R⁷ is isopropyl; and R⁹ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is C₁₋₆haloalkyl; and R⁹ is H. In some embodiments, R⁷ is C₁₋₃haloalkyl; and R⁹ is H. In some embodiments, R⁷ is CF₃; and R⁹ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ and R⁹ are each halo. In some embodiments, R⁷ is Cl and R⁹ is F. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A3), or a stereoisomer or tautomer thereof, or a pharmaceutically acceptable salt of any of the foregoing, R⁷ is H; and R⁹ is halo. In some embodiments, R⁷ is H; and R⁹ is F. In some embodiments, R⁷ is H; and R⁹ is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-A4):

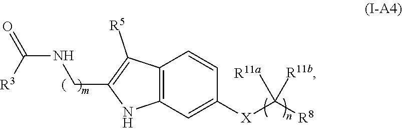

or a stereoisomer or tautomer thereof, or a pharmaceutically acceptable salt of any of the foregoing, wherein m, n, R³, R⁵, R⁸, R^{11a}, R^{11b}, and X are as defined for a compound of formula (I). In some variations, m, n, R³, R⁵, R⁸, R^{11a}, R^{11b}, and X of formula (I-A4) are as defined for a compound of formula (I′), or a stereoisomer or tautomer thereof, or a pharmaceutically acceptable salt of any of the foregoing, or any variation or embodiment thereof.

In some embodiments of a compound of formula (I), or (I-A), such as a compound of formula (I-A4), or a stereoisomer or tautomer thereof, or a pharmaceutically acceptable salt of any of the foregoing, R⁵ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A4), or a stereoisomer or tautomer thereof, or a pharmaceutically acceptable salt of any of the foregoing, R⁵ is C₁₋₆alkyl. In some embodiments, R⁵ is C₁₋₃alkyl. In some embodiments, R⁵ is methyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A4), or a stereoisomer or tautomer thereof, or a pharmaceutically acceptable salt of any of the foregoing, R⁵ is halo. In some embodiments, R⁵ is F. In some embodiments, R⁵ is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-A5):

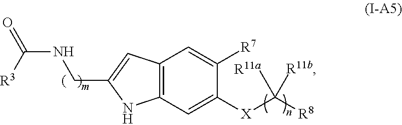

or a stereoisomer or tautomer thereof, or a pharmaceutically acceptable salt of any of the foregoing, wherein m, n, R², R³, R⁷, R⁸, R¹⁰, R^{11a}, R^{11b}, and X are as defined for a compound of formula (I). In some variations, m, n, R², R³, R⁷, R⁸, R¹⁰, R^{11a}, R^{11b}, and X of formula (I-A5) are as defined for a compound of formula (I′), or a stereoisomer or tautomer thereof, or a pharmaceutically acceptable salt of any of the foregoing, or any variation or embodiment thereof.

In some embodiments of a compound of formula (I), or (I-A), such as a compound of formula (I-A5), or a stereoisomer or tautomer thereof, or a pharmaceutically acceptable salt of any of the foregoing, R⁷ is H, halo, C₁₋₆ alkyl, C₃₋₁₀cycloalkyl or C₁₋₆ haloalkyl, wherein the C₁₋₆ alkyl of R⁷ is optionally substituted with one or more halo, or CN. In some embodiments, R⁷ is H, halo, C₁₋₃alkyl, C₃₋₆cycloalkyl or C₁₋₃haloalkyl, wherein the C₁₋₃alkyl of R⁷ is optionally substituted with one or more halo, or CN. In some embodiments, R⁷ is H, halo, C₁₋₃alkyl, or C₁₋₃haloalkyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A5), or a stereoisomer or tautomer thereof, or a pharmaceutically acceptable salt of any of the foregoing, R⁷ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A5), or a stereoisomer or tautomer thereof, or a pharmaceutically acceptable salt of any of the foregoing, R⁷ is halo. In some embodiments, R⁷ is F. In some embodiments, R⁷ is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A5), or a stereoisomer or tautomer thereof, or a pharmaceutically acceptable salt of any of the foregoing, R⁷ is C₁₋₆alkyl. In some embodiments, R⁷ is C₁₋₃alkyl. In some embodiments, R⁷ is methyl. In some embodiments, R⁷ is ethyl. In some embodiments, R⁷ is isopropyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), or (I-A), such as a compound of formula (I-A5), or a stereoisomer or tautomer thereof, or a pharmaceutically acceptable salt of any of the foregoing, R⁷ is C₁₋₆haloalkyl. In some embodiments, R⁷ is C₁₋₃haloalkyl. In some embodiments, R⁷ is CF₃. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-A6):

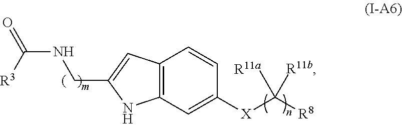

or a stereoisomer or tautomer thereof, or a pharmaceutically acceptable salt of any of the foregoing, wherein m, n, R³, R⁸, R¹⁰, R^{11a}, R^{11b}, and X are as defined for a compound of formula (I). In some variations, m, n, R³, R⁸, R¹⁰, R^{11a}, R^{11b}, and X of formula (I-A6) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-B):

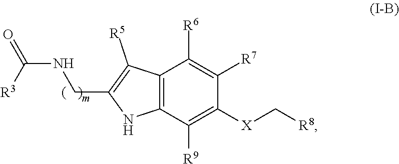

or a stereoisomer or tautomer thereof, or a pharmaceutically acceptable salt of any of the foregoing, wherein m, R², R³, R⁵, R⁶, R⁷, R⁸, R⁹, R¹⁰, and X are as defined for a compound of formula (I). In some variations, m, R², R³, R⁵, R⁶, R⁷, R⁸, R⁹, R¹⁰, and X of formula (I-B) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), (I-A), or (I-B), such as a compound of formula (I-B1):

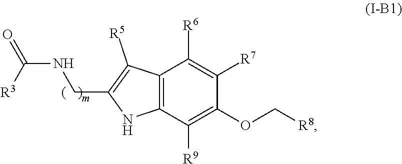

or a stereoisomer or tautomer thereof, or a pharmaceutically acceptable salt of any of the foregoing, wherein m, R³, R⁵, R⁶, R⁷, R⁸, and R⁹ are as defined for a compound of formula (I). In some variations, m, R³, R⁵, R⁶, R⁷, R⁸, and R⁹ of formula (I-B1) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), (I-A), or (I-B), such as a compound of formula (I-B2):

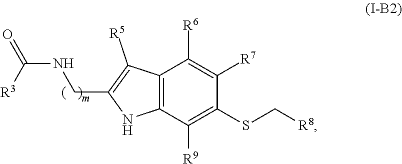

or a stereoisomer or tautomer thereof, or a pharmaceutically acceptable salt of any of the foregoing, wherein m, R³, R⁵, R⁶, R⁷, R⁸, and R⁹ are as defined for a compound of formula (I). In some variations, m, R³, R⁵, R⁶, R⁷, R⁸, and R⁹ of formula (I-B2) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), (I-A), or (I-B), such as a compound of formula (I-B3):

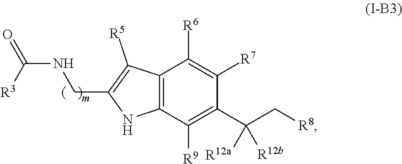

or a stereoisomer or tautomer thereof, or a pharmaceutically acceptable salt of any of the foregoing, wherein m, R³, R⁵, R⁶, R⁷, R⁸, and R⁹, are as defined for a compound of formula (I). In some variations, m, R³, R⁵, R⁶, R⁷, R⁸, and R⁹ of formula (I-B3) are as defined for a compound of formula (I′), or a stereoisomer or tautomer thereof, or a pharmaceutically acceptable salt of any of the foregoing, or any variation or embodiment thereof.

In some embodiments of a compound of formula (I), (I-A), or (I-B), such as a compound of formula (I-B3), or a stereoisomer or tautomer thereof, or a pharmaceutically acceptable salt of any of the foregoing, R⁴ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), (I-A), or (I-B), such as a compound of formula (I-B3), or a stereoisomer or tautomer thereof, or a pharmaceutically acceptable salt of any of the foregoing, R^{12a} and R^{12b} are each independently, H, or C₁₋₆alkyl. In some embodiments, R^{12a} and R^{12b} are each independently, H, or C₁₋₃alkyl. In some embodiments, R^{12a} and R^{12b} are each independently, H. In some embodiments, one of R^{12a} and R^{12b} is H, and the other of R^{12a} and R^{12b} is C₁₋₃alkyl. In some embodiments, one of R^{12a} and R^{12b} is H, and the other of R^{12a} and R^{12b} is methyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), (I-A), or (I-B), such as a compound of formula (I-B4):

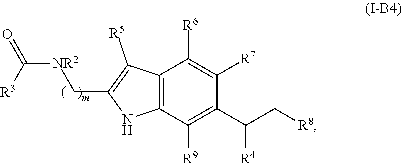

or a stereoisomer or tautomer thereof, or a pharmaceutically acceptable salt of any of the foregoing, wherein m, R³, R⁵, R⁶, R⁷, R⁸, and R⁹ are as defined elsewhere herein. In some variations, m, R³, R⁵, R⁶, R⁷, R⁸, and R⁹ of formula (I-B4) are as defined for a compound of formula (I′), or a stereoisomer or tautomer thereof, or a pharmaceutically acceptable salt of any of the foregoing, or any variation or embodiment thereof.

In some embodiments of a compound of formula (I), (I-A), or (I-B), such as a compound of formula (I-B4), or a stereoisomer or tautomer thereof, or a pharmaceutically acceptable salt of any of the foregoing, R⁴ is H. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), (I-A), or (I-B), such as a compound of formula (I-B4), or a stereoisomer or tautomer thereof, or a pharmaceutically acceptable salt of any of the foregoing, R⁴ is C₁₋₆alkyl. In some embodiments, R⁴ is C₁₋₃alkyl. In some embodiments, R⁴ is CH₃. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), such as a compound of formula (I-C):

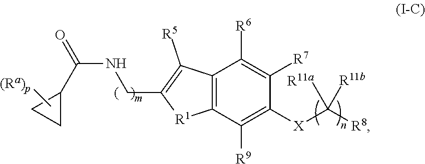

or a stereoisomer or tautomer thereof, or a pharmaceutically acceptable salt of any of the foregoing, wherein m, n, R^{a}, R¹, R⁵, R⁶, R⁷, R⁸, R⁹, R^{11a}, R^{11b}, and X are as defined for a compound of formula (I); and p is an integer from 0 to 5. In some variations, m, n, R^{a}, R¹, R⁵, R⁶, R⁷, R⁸, R⁹, R^{11a}, R^{11b}, and X of formula (I-C) are as defined for a compound of formula (I′), or a stereoisomer or tautomer thereof, or a pharmaceutically acceptable salt of any of the foregoing, or any variation or embodiment thereof.

In some embodiments of a compound of formula (I), such as a compound of formula (I-C), or a stereoisomer or tautomer thereof, or a pharmaceutically acceptable salt of any of the foregoing, p is 0 or 1. In some embodiments, p is 0. In some embodiments, p is 1. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), or (I-C), such as a compound of formula (I-C1):

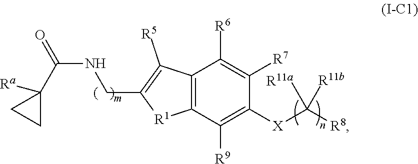

or a stereoisomer or tautomer thereof, or a pharmaceutically acceptable salt of any of the foregoing, wherein m, n, R^{a}, R¹, R⁵, R⁶, R⁷, R⁸, R⁹, R¹⁰, R^{1a}, R^{11b} and X are as defined for a compound of formula (I). In some variations, m, n, R^{a}, R¹, R⁵, R⁶, R⁷, R⁸, R⁹, R¹⁰, R^{1a}, R^{11b} and X of formula (I-C1) are as defined for a compound of formula (I′), or a stereoisomer or tautomer thereof, or a pharmaceutically acceptable salt of any of the foregoing, or any variation or embodiment thereof.

In some embodiments of a compound of formula (I), or (I-C), such as a compound of formula (I-C1), or a stereoisomer or tautomer thereof, or a pharmaceutically acceptable salt of any of the foregoing, R¹ is —NH—. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), (I-A), or (I-C), such as a compound of formula (I-C2):

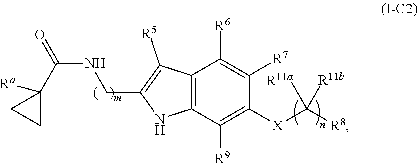

or a stereoisomer or tautomer thereof, or a pharmaceutically acceptable salt of any of the foregoing, wherein m, n, R^{a}, R⁵, R⁶, R⁷, R⁸, R⁹, R^{11a}, R^{11b}, and X are as defined for a compound of formula (I). In some variations, m, n, R^{a}, R⁵, R⁶, R⁷, R⁸, R⁹, R^{11a}, R^{11b}, and X of formula (I-C2) are as defined for a compound of formula (I′), or a stereoisomer or tautomer thereof, or a pharmaceutically acceptable salt of any of the foregoing, or any variation or embodiment thereof.

In some embodiments of a compound of formula (I), (I-A), or (I-C), such as a compound of formula (I-C2), or a stereoisomer or tautomer thereof, or a pharmaceutically acceptable salt of any of the foregoing, R^{a} is —OH, halo, C₁₋₆alkyl, C₁₋₆haloalkyl, or C₃₋₁₀cycloalkyl. In some embodiments, R^{a} is —OH, halo, C₁₋₃alkyl, C₁₋₃haloalkyl, or C₃₋₆cycloalkyl. In some embodiments, R^{a} is C₁₋₃alkyl, or C₁₋₃haloalkyl. In some embodiments, R^{a} is CH₃, or CF₃. In some embodiments, R^{a} is CH₃. In some embodiments, R^{a} is halo. In some embodiments, R^{a} is Br, Cl, F, or I. In some embodiments, R^{a} is Cl. In some embodiments, R^{a} is F. In some embodiments, R^{a} is Br. In some embodiments of a compound of formula (I), (I-A), or (I-C), such as a compound of formula (I-C2), or a stereoisomer or tautomer thereof, or a pharmaceutically acceptable salt of any of the foregoing, R^{a} is —OH, oxo, —CN, halo, C₁₋₆alkyl, C₁₋₆ alkoxy, C₁₋₆ haloalkyl, —O—C₁₋₆ haloalkyl, —NH₂, —NH(C₁₋₆alkyl), —N(C₁₋₆alkyl)₂, 4-10 membered heterocyclyl, or C₃₋₁₀cycloalkyl, wherein the C₁₋₆alkyl, C₁₋₆alkoxy, 4-10 membered heterocyclyl or C₃₋₁₀cycloalkyl of R^{a} is optionally substituted with one or more R^{b}; and each R^{b} is, independently at each occurrence, —OH, halo, C₁₋₆alkyl or C₁₋₆alkoxy. In some embodiments, R^{a} is —OH, oxo, —CN, halo, C₁₋₃alkyl, C₁₋₃alkoxy, C₁₋₃haloalkyl, —O—C₁₋₃haloalkyl, —NH₂, —NH(C₁₋₆alkyl), —N(C₁₋₆ alkyl)₂, 4-6 membered heterocyclyl, or C₃₋₆cycloalkyl, wherein the C₁₋₃alkyl, C₁₋₃alkoxy, 4-10 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy. In some embodiments, R^{a} is —OH, halo, C₁₋₃alkyl, C₁₋₃haloalkyl, wherein the C₁₋₃alkyl of R^{a} is optionally substituted with one or more R^{b}; and each R^{b} is, independently at each occurrence, —OH, or C₁₋₃alkoxy. In some embodiments, R^{a} is selected from the group consisting of

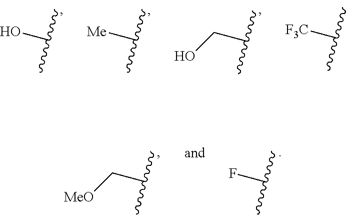

In some embodiments of a compound of formula (I), (I-A), or (I-C), such as a compound of formula (I-C2), or a stereoisomer or tautomer thereof, or a pharmaceutically acceptable salt of any of the foregoing, X is —O—, —S—, —C(R^{12a})(R^{12b})—, —N(H)—, or —N(C₁₋₆ alkyl)-. In some embodiments, X is —O—, —S—, —C(R^{12a})(R^{12b})—, —N(H)—, or —N(C₁₋₃alkyl)-. In some embodiments, X is —O—. In some embodiments, X is —S—. In some embodiments, X is —NH—. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), (I-A), or (I-C), such as a compound of formula (I-C3):

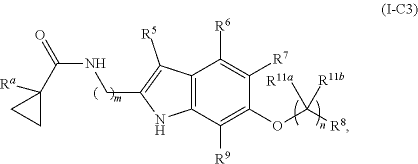

or a stereoisomer or tautomer thereof, or a pharmaceutically acceptable salt of any of the foregoing, wherein m, n, R^{a}, R⁵, R⁶, R⁷, R⁸, R⁹, R^{11a}, and R^{11b} are as defined for a compound of formula (I). In some variations, m, n, R^{a}, R⁵, R⁶, R⁷, R⁸, R⁹, R^{11a}, and R^{11b} of formula (I-C3) are as defined for a compound of formula (I′), or a stereoisomer or tautomer thereof, or a pharmaceutically acceptable salt of any of the foregoing, or any variation or embodiment thereof.

In some embodiments of a compound of formula (I), (I-A), or (I-C), such as a compound of formula (I-C2), or (I-C3), or a stereoisomer or tautomer thereof, or a pharmaceutically acceptable salt of any of the foregoing, X is —O—, and R^{a} is C₁₋₆alkyl. In some embodiments, X is —O—, and R^{a} is C₁₋₃alkyl. In some embodiments, X is —O—, and R^{a} is methyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), (I-A), or (I-C), such as a compound of formula (I-C2), or (I-C3), or a stereoisomer or tautomer thereof, or a pharmaceutically acceptable salt of any of the foregoing, X is —O—, and R^{a} is halo. In some embodiments, X is —O—, and R^{a} is Br, Cl, F, or I. In some embodiments, X is —O—, and R^{a} is F. In some embodiments, X is —O—, and R^{a} is Cl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I′), (I-A), or (I-C), such as a compound of formula (I-C2), or (I-C3), or a stereoisomer or tautomer thereof, or a pharmaceutically acceptable salt of any of the foregoing, X is —O—, and R^{a} is —OH, halo, C₁₋₆alkyl, C₁₋₆haloalkyl, wherein the C₁₋₆alkyl of R^{a} is optionally substituted with one or more R^{b}; and each R^{b} is, independently at each occurrence, —OH, or C₁₋₆alkoxy. In some embodiments, X is —O—, and R^{a} is —OH, halo, C₁₋₃alkyl, C₁₋₃haloalkyl, wherein the C₁₋₃alkyl of R^{a} is optionally substituted with one or more R^{b}; and each R^{b} is, independently at each occurrence, —OH, or C₁₋₃alkoxy.

In one aspect, provided herein is a compound of formula (I-C4):

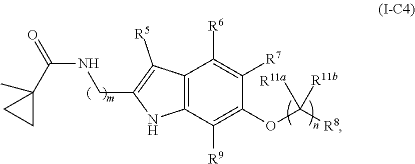

or a stereoisomer or tautomer thereof, or a pharmaceutically acceptable salt of any of the foregoing, wherein m, n, R⁵, R⁶, R⁷, R⁸, R⁹, R^{11a}, and R^{11b} are as defined for a compound of formula (I). In some variations, m, n, R⁵, R⁶, R⁷, R⁸, R⁹, R^{11a}, and R^{11b} of formula (I-C4) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I-C5):

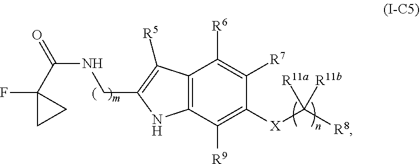

or a stereoisomer or tautomer thereof, or a pharmaceutically acceptable salt of any of the foregoing, wherein m, n, R⁵, R⁶, R⁷, R⁸, R⁹, R^{11a}, and R^{11b} are as defined for a compound of formula (I). In some variations, m, n, R⁵, R⁶, R⁷, R⁸, R⁹, R^{11a}, and R^{11b} of formula (I-C5) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), or (I-A), such as a compound of formula (I-D):

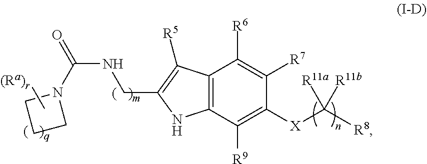

or a stereoisomer or tautomer thereof, or a pharmaceutically acceptable salt of any of the foregoing, wherein m, n, q, r, R^{a}, R⁵, R⁶, R⁷, R⁸, R⁹, R^{11a}, R^{11b} and X are as defined for a compound of formula (I); q is an integer from 1-7; and r is an integer from 0-18. In some variations, m, n, q, r, R^{a}, R⁵, R⁶, R⁷, R⁸, R⁹, R^{11a}, R^{11b} and X of formula (I-D) are as defined for a compound of formula (I′), or a stereoisomer or tautomer thereof, or a pharmaceutically acceptable salt of any of the foregoing, or any variation or embodiment thereof.

In some embodiments of a compound of formula (I), such as a compound of formula (I-D), or a stereoisomer or tautomer thereof, or a pharmaceutically acceptable salt of any of the foregoing, q is an integer from 1 to 3. In some embodiments, q is 1. In some embodiments, q is 2. In some embodiments, q is 3. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), (I-A), or (I-D), such as a compound of formula (I-D1):

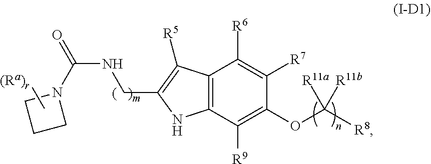

or a stereoisomer or tautomer thereof, or a pharmaceutically acceptable salt of any of the foregoing, wherein m, n, R^{a}, R⁵, R⁶, R⁷, R⁸, R⁹, R^{11a}, and R^{11b} are as defined for a compound of formula (I); and r is an integer from 0 to 6. In some variations, m, n, R^{a}, R⁵, R⁶, R⁷, R⁸, R⁹, R^{11a}, and R^{11b} of formula (I-D1) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), (I-A), or (I-D), such as a compound of formula (I-D2):

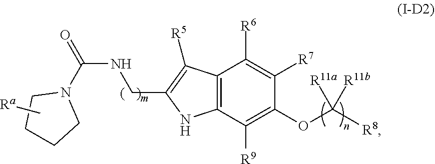

or a stereoisomer or tautomer thereof, or a pharmaceutically acceptable salt of any of the foregoing, wherein m, n, R^{a}, R⁵, R⁶, R⁷, R⁸, R⁹, R^{11a}, and R^{11b} are as defined for a compound of formula (I); and r is an integer from 0 to 8. In some variations m, n, R^{a}, R⁵, R⁶, R⁷, R⁸, R⁹, R^{11a}, and R^{11b} of formula (I-D2) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I), (I-A), or (I-D), such as a compound of formula (I-D3):

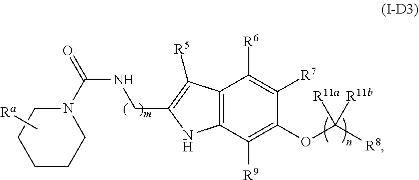

or a stereoisomer or tautomer thereof, or a pharmaceutically acceptable salt of any of the foregoing, wherein m, n, R^{a}, R⁵, R⁶, R⁷, R⁸, R⁹, R^{11a}, and R^{11b} are as defined for a compound of formula (I); and r is an integer from 0 to 10. In some variations, m, n, R^{a}, R⁵, R⁶, R⁷, R⁸, R⁹, R^{11a}, and R^{11b} of formula (I-D3) are as defined for a compound of formula (I′), or a stereoisomer or tautomer thereof, or a pharmaceutically acceptable salt of any of the foregoing, or any variation or embodiment thereof. In some variations, m, n, R^{a}, R⁵, R⁶, R⁷, R⁸, R⁹, R^{11a}, and R^{11b} of formula (I-D3) are as defined for a compound of formula (I′), or a stereoisomer or tautomer thereof, or a pharmaceutically acceptable salt of any of the foregoing, or any variation or embodiment thereof.

In one aspect, provided herein is a compound of formula (II), such as a compound of formula (I-D1):

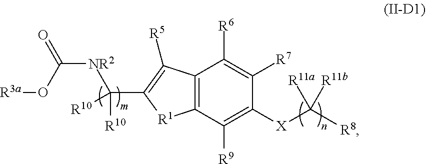

or a stereoisomer or tautomer thereof, or a pharmaceutically acceptable salt of any of the foregoing, wherein m, n, R⁵, R⁶, R⁷, R⁸, R⁹, R¹⁰, R^{11a}, and R^{11b} are as defined for a compound of formula (I); and R^{3a} is C₁₋₆alkyl.

In one aspect, provided is a compound of formula (I), such as a compound of formula (I-E):

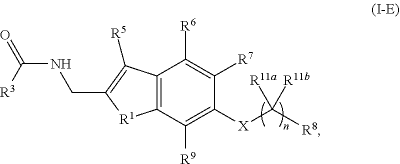

or a stereoisomer or tautomer thereof, or a pharmaceutically acceptable salt of any of the foregoing, wherein m, n, R¹, R³, R⁵, R⁶, R⁷, R⁸, R⁹, R^{11a}, R^{11b} and X are as defined elsewhere herein. In some variations, m, n, R¹, R³, R⁵, R⁶, R⁷, R⁸, R⁹, R^{11a}, R^{11b} and X of formula (I-E) are as defined for a compound of formula (I′), or a stereoisomer or tautomer thereof, or a pharmaceutically acceptable salt of any of the foregoing, or any variation or embodiment thereof.

In some embodiments of a compound of formula (I), such as a compound of formula (I-E), or a stereoisomer or tautomer thereof, or a pharmaceutically acceptable salt of any of the foregoing, n is an integer from 0 to 4. In some embodiments, n is 0. In some embodiments, n is 1. In some embodiments, n is 2. In some embodiments, n is 3. In some embodiments, n is 4. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided is a compound of formula (I), or (I-E), such as a compound of formula (I-E1):

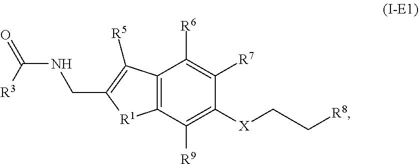

or a stereoisomer or tautomer thereof, or a pharmaceutically acceptable salt of any of the foregoing, wherein m, R³, R⁵, R⁶, R⁷, R⁸, R⁹, and X are as defined elsewhere herein. In some variations, m, R³, R⁵, R⁶, R⁷, R⁸, R⁹, and X of formula (I-E1) are as defined for a compound of formula (I′), or a stereoisomer or tautomer thereof, or a pharmaceutically acceptable salt of any of the foregoing, or any variation or embodiment thereof.

In one aspect, provided is a compound of formula (I-E2):

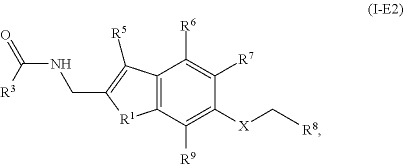

or a stereoisomer or tautomer thereof, or a pharmaceutically acceptable salt of any of the foregoing, wherein m, R³, R⁵, R⁶, R⁷, R⁸, R⁹, and X are as defined elsewhere herein. In some variations, m, R³, R⁵, R⁶, R⁷, R⁸, R⁹, and X of formula (I-E2) are as defined for a compound of formula (I′), or a stereoisomer or tautomer thereof, or a pharmaceutically acceptable salt of any of the foregoing, or any variation or embodiment thereof.

In one aspect, provided is a compound of formula (I-E3):

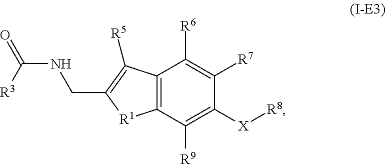

or a stereoisomer or tautomer thereof, or a pharmaceutically acceptable salt of any of the foregoing, wherein m, R³, R⁵, R⁶, R⁷, R⁸, R⁹, and X are as defined elsewhere herein. In some variations, m, R³, R⁵, R⁶, R⁷, R⁸, R⁹, and X of formula (I-E3) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I-F):

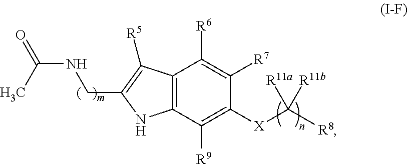

or a stereoisomer or tautomer thereof, or a pharmaceutically acceptable salt of any of the foregoing, wherein m, n, R⁵, R⁶, R⁷, R⁸, R⁹, R^{11a}, R^{11b}, and X are as defined for a compound of formula (I). In some variations, m, R³, R⁵, R⁶, R⁷, R⁸, R⁹, and X of formula (I-E3) are as defined for a compound of formula (I′), or a stereoisomer or tautomer thereof, or a pharmaceutically acceptable salt of any of the foregoing, or any variation or embodiment thereof.

In one aspect, provided herein is a compound of formula (I-G):

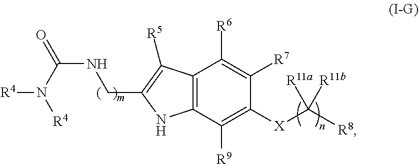

or a stereoisomer or tautomer thereof, or a pharmaceutically acceptable salt of any of the foregoing, wherein m, n, each R⁴, R⁵, R⁶, R⁷, R⁸, R⁹, R^{11a}, R^{11b}, and X are as defined for a compound of formula (I). In some variations, m, n, each R⁴, R⁵, R⁶, R⁷, R⁸, R⁹, R^{11a}, R^{11b}, and X of formula (I-G) are as defined for a compound of formula (I′), or a stereoisomer or tautomer thereof, or a pharmaceutically acceptable salt of any of the foregoing, or any variation or embodiment thereof.

In some embodiments of a compound of formula (I), such as a compound of formula (I-G), or a stereoisomer or tautomer thereof, or a pharmaceutically acceptable salt of any of the foregoing, each R⁴ is independently H, C₁₋₆alkyl, C₁₋₆haloalkyl, or C₃₋₁₀cycloalkyl. In some embodiments, each R⁴ is independently H, C₁₋₃alkyl, C₁₋₃haloalkyl, or C₃₋₆cycloalkyl. In some embodiments, each R⁴ is independently H, or C₁₋₃alkyl. In some embodiments, one R⁴ is H and the other R⁴ is C₁₋₃alkyl. In some embodiments, one R⁴ is H and the other R⁴ is methyl. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I-H):

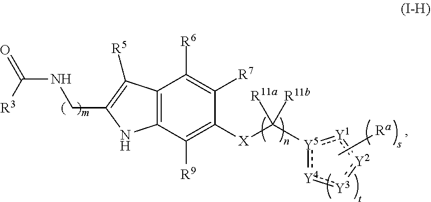

or a stereoisomer or tautomer thereof, or a pharmaceutically acceptable salt of any of the foregoing, wherein m, n, R³, R⁵, R⁶, R⁷, R⁹, R^{11a}, R^{11b}, R^{a} and X are as defined for a compound of formula (I); Y¹, Y², Y³, Y⁴, and Y⁵, are each independently —C—, —CH—, —CH₂—, —N—, —NH—, —S—, or —O—; s is an integer from 0-6; t is 1 or 2; and [symbol] represents a single or double bond. In some variations, m, n, R³, R⁵, R⁶, R⁷, R⁹, R^{11a}, R^{11b}, R^{a} and X of formula (I-H) are as defined for a compound of formula (I′), or a stereoisomer or tautomer thereof, or a pharmaceutically acceptable salt of any of the foregoing, or any variation or embodiment thereof.

In some embodiments of a compound of formula (I), such as a compound of formula (I-H), or a stereoisomer or tautomer thereof, or a pharmaceutically acceptable salt of any of the foregoing, m is an integer from 1 to 2; n is an integer from 0 to 2; X is —O—, —S—, —C(R^{12a})(R^{12b})—, —N(H)—, or —N(C₁₋₆alkyl)-; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₃alkyl, C₁₋₃haloalkyl, or C₃₋₆cycloalkyl, or both R⁴ are taken together with the N atom to which they are attached to form a 4-10 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN; R⁶ and R⁹ are each independently H, or halo; R^{11b}a and R^{11b} are each independently at each occurrence, H, or C₁₋₃alkyl, or taken together with one of R^{12a} or R^{12b} to form a C₃₋₆cycloalkyl; R^{12a} and R^{12b} are each independently, H, or C₁₋₃alkyl, or one of R^{12a} and R^{12b} is H, or C₁₋₃alkyl, and the other of R^{12a} and R^{12b} is taken together with R^{11b}a or R^{11b} to form a C₃₋₆cycloalkyl; each R^{a} is, independently at each occurrence, —OH, halo, C₁₋₃alkyl, C₁₋₃haloalkyl, or C₃₋₆cycloalkyl; Y¹, Y², Y³, Y⁴, and Y⁵, are each independently —C—, —CH—, —CH₂—, —N—, —NH—, —S—, or —O—; s is an integer from 0-6; t is 1 or 2; and [symbol] represents a single or double bond.

In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), such as a compound of formula (I-H), or a stereoisomer or tautomer thereof, or a pharmaceutically acceptable salt of any of the foregoing, m is an integer from 1 to 2; n is an integer from 0 to 2; X is —O—, —S—, —C(R^{12a})(R^{12b})—, —N(H)—, or —N(C₁₋₆alkyl)-; R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}; each R⁴ is independently H, C₁₋₃alkyl, C₁₋₃haloalkyl, or C₃₋₆cycloalkyl, or both R⁴ are taken together with the N atom to which they are attached to form a 4-6 membered heterocyclyl optionally substituted with one or more R^{a}; R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo or CN; R⁶ and R⁹ are each independently H, or halo; R^{11a} and R^{11b} are each independently at each occurrence, H or C₁₋₃alkyl, one of R^{11a} and R^{11b} is H or C₁₋₆alkyl, and the other of R^{11a} and R^{11b} is taken together with one of R^{12a} or R^{12b} to form a C₃₋₁₀cycloalkyl; R^{12a} and R^{12b} are each independently, H, or C₁₋₃alkyl, or one of R^{12a} and R^{12b} is H, or C₁₋₃alkyl, and the other of R^{12a} and R^{12b} is taken together with R^{11a} or R^{11b} to form a C₃₋₆cycloalkyl; each R^{a} is, independently at each occurrence, —OH, oxo, —CN, halo, C₁₋₃alkyl, C₁₋₃alkoxy, C₁₋₃haloalkyl, —O—C₁₋₃haloalkyl, —NH₂, —NH(C₁₋₃alkyl), —N(C₁₋₃alkyl)₂, 4-6 membered heterocyclyl, or C₃₋₆cycloalkyl, wherein the C₁₋₃alkyl, C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy; Y¹, Y², Y³, Y⁴, and Y⁵, are each independently —C—, —CH—, —CH₂—, —N—, —NH—, —S—, or —O—; s is an integer from 0-6; t is 1 or 2; and [symbol] represents a single or double bond.

In some embodiments of a compound of formula (I), such as a compound of formula (I-H), or a stereoisomer or tautomer thereof, or a pharmaceutically acceptable salt of any of the foregoing, the compound is compound of formula:

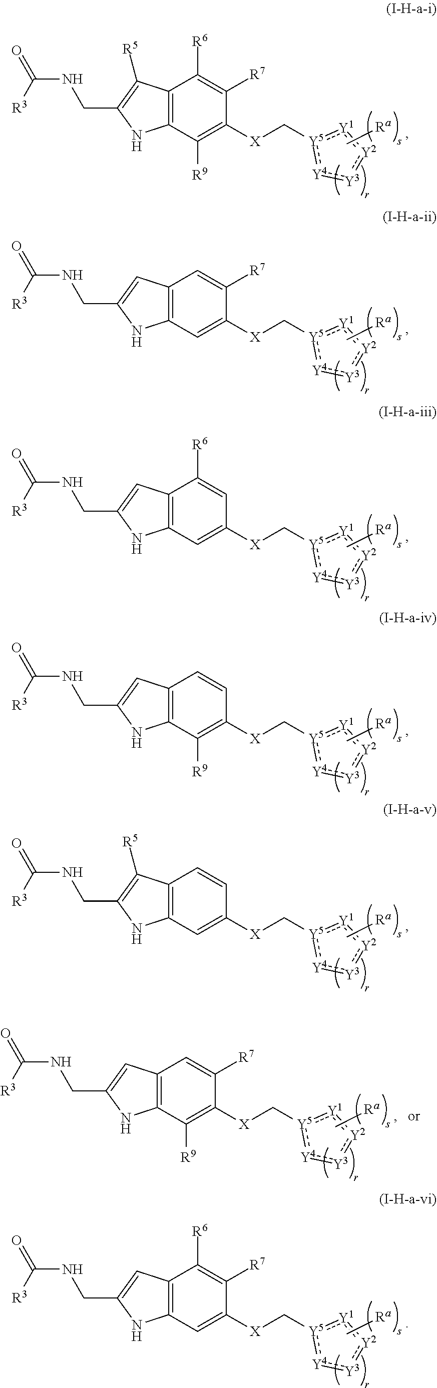

In some embodiments t is 0. In some embodiments, t is 1. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In some embodiments of a compound of formula (I), such as a compound of formula (I-H), or a stereoisomer or tautomer thereof, or a pharmaceutically acceptable salt of any of the foregoing, the compound is compound of formula:

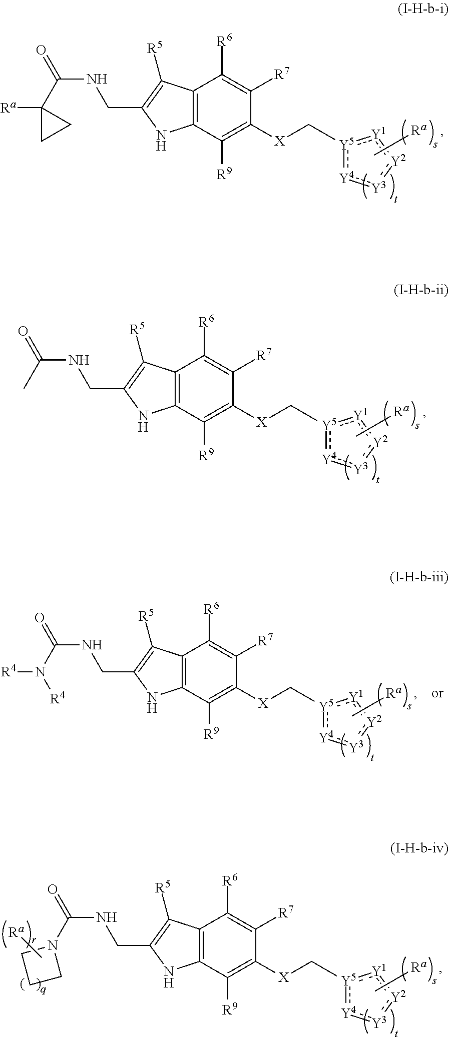

wherein q is an integer from 1-7; and r is an integer from 0-18. In some embodiments t is 0. In some embodiments, t is 1. In some embodiments, t is 1, and Y⁵ is —C—. In some embodiments, t is 1, Y⁵ is —C—, any one of Y¹, Y², Y³, and Y⁴ is —N—, —NH—, —S—, or —O— and the others of Y¹, Y², Y³, and Y⁴ are each independently —C—, —CH—, or —CH₂—. In some embodiments, t is 1, Y⁵ is —C—, any two of Y¹, Y², Y³, and Y⁴ is —N—, —NH—, —S—, or —O— and the others of Y¹, Y², Y³, and Y⁴ are each independently —C—, —CH—, or —CH₂—. In some variations, the embodiments provided herein also apply to any other applicable formula detailed herein, such as a compound of formula (I′) or (II) or a stereoisomer or tautomer thereof, or a pharmaceutically acceptable salt of any of the foregoing, or any variation or embodiment thereof.

In one aspect, provided herein is a compound of formula (I), such as a compound of formula (I-A):

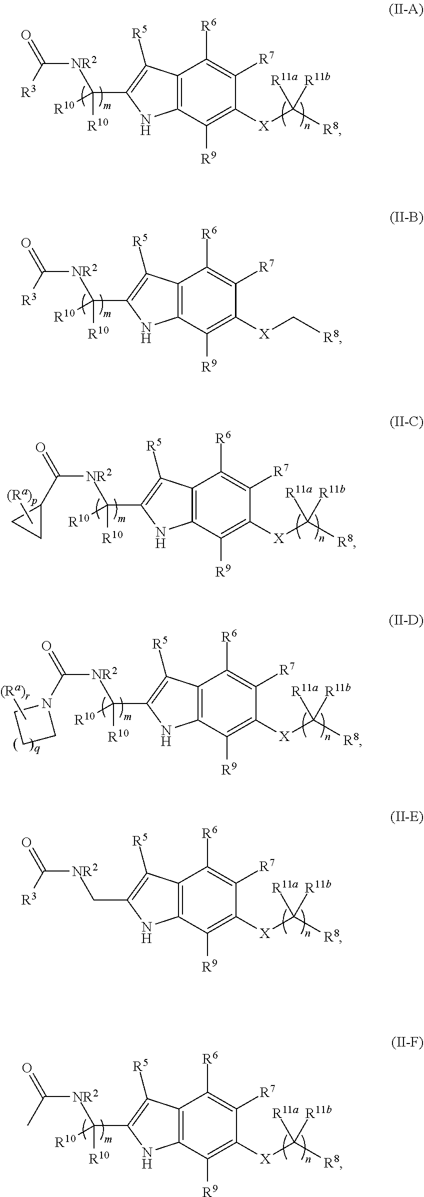

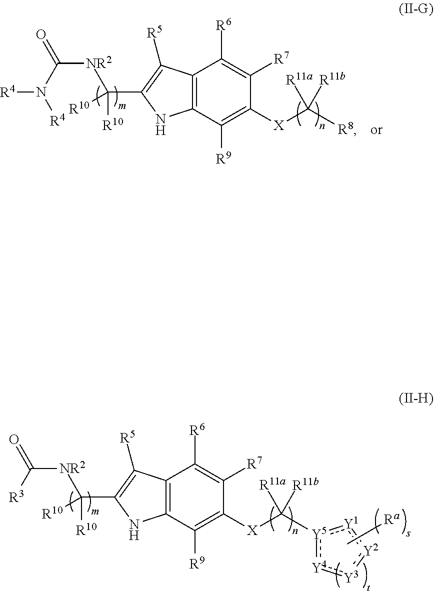

or a stereoisomer or tautomer thereof, or a pharmaceutically acceptable salt of any of the foregoing, or tautomer thereof, or a pharmaceutically acceptable salt of any of the foregoing, wherein m, n, p, q, r, s, t, R^{a}, R³, each R⁴, R⁵, R⁶, R⁷, R⁸, R⁹, R^{11a}, R^{11b}, X, Y¹, Y², Y³, Y⁴ and Y are as defined elsewhere herein for a compound of formula (II), (I-A), (I-B), (I-C), (I-D), (I-E), (I-F), or (I-G), (I-H).

In some embodiments of a compound of formula (I), or (I′) or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1. In some embodiments of a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-204 of Table 1. In some embodiments of a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-236 of Table 1. In some embodiments of a compound of formula (I′), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-317 of Table 1. In some embodiments of a compound of formula (II), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-669 of Table 1.

Compound Names included in Table 1 and for all intermediates and compounds were generated using ChemDraw® Professional software version 17.1.1.0 or Collaborative Drug Discovery Inc. (CDD) CDD Vault update #3.

A Knime workflow was created to retrieve structures from an internal ChemAxon Compound Registry, generate the canonical smiles using RDKit Canon SMILES node, remove the stereochemistry using ChemAxon/Infocom MolConverter node, and name the structure using ChemAxon/Infocom Naming node. The following denotes the version of the Knime Analytics Platform and extensions utilized in the workflow:
Knime Analytics Platform 4.2.2
RDKit Knime Integration 4.0.2.v202006261025 (this extension includes the RDKit Canon SMILES node)
ChemAxon/Infocom Marvin Extensions Feature 4.3.0v202100 (this extension includes the MolConverter node)
ChemAxon/Infocom JChem Extensions Feature 4.3.0v202100 (this extension includes the Naming node)

TABLE 1

| Cmpd | | |
|---|---|---|
| No. | Structure | IUPAC name |
| 1 | 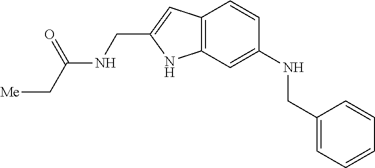 [SMILES: CCC(=O)NCc1cc2ccc(NCc3ccccc3)cc2[nH]1] | N-((6-(benzylamino)-1H-indol-2- yl)methyl)propionamide |
| 2 | 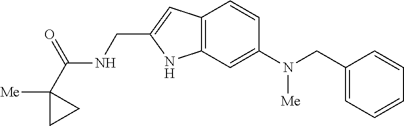 [SMILES: CN(Cc1ccccc1)c1ccc2cc(CNC(=O)C3(C)CC3)[nH]c2c1] | N-((6-(benzyl(methyl)amino)-1H-indol- 2-yl)methyl)-1-methylcyclopropane-1- carboxamide |
| 3 | 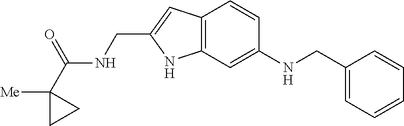 [SMILES: CC1(C(=O)NCc2cc3ccc(NCc4ccccc4)cc3[nH]2)CC1] | N-((6-(benzylamino)-1H-indol-2- yl)methyl)-1- methylcyclopropanecarboxamide |
| 4 | 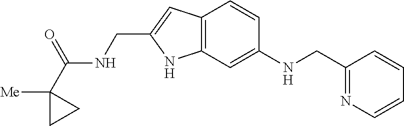 [SMILES: CC1(C(=O)NCc2cc3ccc(NCc4ccccn4)cc3[nH]2)CC1] | 1-methyl-N-((6-((pyridin-2- ylmethyl)amino)-1H-indol-2- yl)methyl)cyclopropane-1-carboxamide |
| 5 | 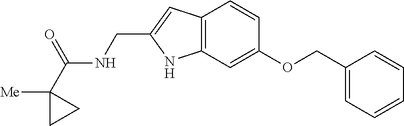 [SMILES: CC1(C(=O)NCc2cc3ccc(OCc4ccccc4)cc3[nH]2)CC1] | N-((6-(benzyloxy)-1H-indol-2- yl)methyl)-1- methylcyclopropanecarboxamide |
| 6 | 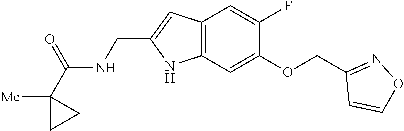 [SMILES: CC1(C(=O)NCc2cc3cc(F)c(OCc4ccon4)cc3[nH]2)CC1] | N-((5-fluoro-6-(isoxazol-3-ylmethoxy)- 1H-indol-2-yl) methyl)-1- methylcyclopropane-1-carboxamide |
| 7 | 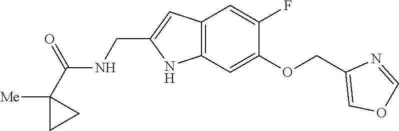 [SMILES: CC1(C(=O)NCc2cc3cc(F)c(OCc4cocn4)cc3[nH]2)CC1] | N-((5-fluoro-6-(oxazol-4-ylmethoxy)- 1H-indol-2-yl)methyl)-1- methylcyclopropane-1-carboxamide |
| 8 | 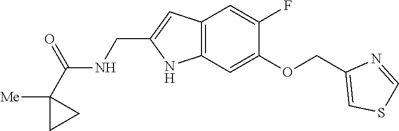 [SMILES: CC1(C(=O)NCc2cc3cc(F)c(OCc4cscn4)cc3[nH]2)CC1] | N-((5-fluoro-6-(thiazol-4-ylmethoxy)- 1H-indol-2-yl)methyl)-1- methylcyclopropane-1-carboxamide |
| 9 | 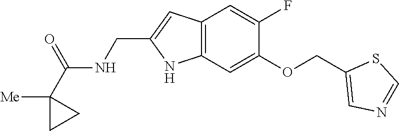 [SMILES: CC1(C(=O)NCc2cc3cc(F)c(OCc4cncs4)cc3[nH]2)CC1] | N-((5-fluoro-6-(thiazol-5-ylmethoxy)- 1H-indol-2-yl)methyl)-1- methylcyclopropane-1-carboxamide |
| 10 | 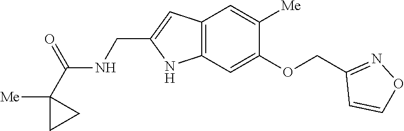 [SMILES: Cc1cc2cc(CNC(=O)C3(C)CC3)[nH]c2cc1OCc1ccon1] | N-((6-(isoxazol-3-ylmethoxy)-5- methyl-1H-indol-2-yl)methyl)-1- methylcyclopropane-1-carboxamide |
| 11 | 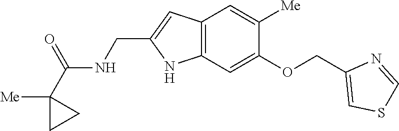 [SMILES: Cc1cc2cc(CNC(=O)C3(C)CC3)[nH]c2cc1OCc1cscn1] | 1-methyl-N-((5-methyl-6-(thiazol-4- ylmethoxy)-1H-indol-2- yl)methyl)cyclopropane-1-carboxamide |
| 12 | 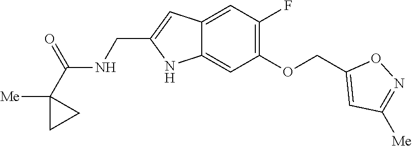 [SMILES: Cc1cc(COc2cc3[nH]c(CNC(=O)C4(C)CC4)cc3cc2F)on1] | N-((5-fluoro-6-((3-methylisoxazol-5- yl)methoxy)-1H-indol-2-yl)methyl)-1- methylcyclopropane-1-carboxamide |
| 13 | 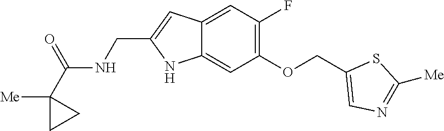 [SMILES: Cc1ncc(COc2cc3[nH]c(CNC(=O)C4(C)CC4)cc3cc2F)s1] | N-((5-fluoro-6-((2-methylthiazol-5- yl)methoxy)-1H-indol-2-yl)methyl)-1- methylcyclopropane-1-carboxamide |
| 14 | 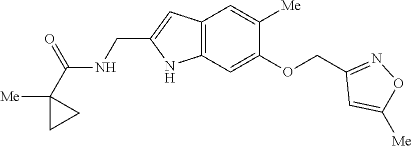 [SMILES: Cc1cc(COc2cc3[nH]c(CNC(=O)C4(C)CC4)cc3cc2C)no1] | 1-methyl-N-((5-methyl-6-((5- methylisoxazol-3-yl)methoxy)-1H- indol-2-yl)methyl)cyclopropane-1- carboxamide |
| 15 | 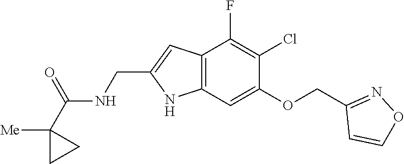 [SMILES: CC1(C(=O)NCc2cc3c(F)c(Cl)c(OCc4ccon4)cc3[nH]2)CC1] | N-((5-chloro-4-fluoro-6-(isoxazol-3- ylmethoxy)-1H-indol-2-yl)methyl)-1- methylcyclopropane-1-carboxamide |
| 16 | 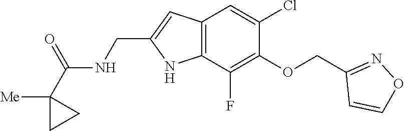 [SMILES: CC1(C(=O)NCc2cc3cc(Cl)c(OCc4ccon4)c(F)c3[nH]2)CC1] | N-((5-chloro-7-fluoro-6-(isoxazol-3- ylmethoxy)-1H-indol-2-yl)methyl)-1- methylcyclopropane-1-carboxamide |
| 17 | 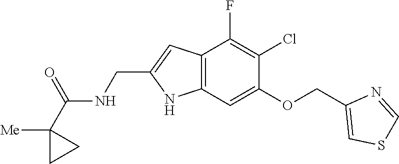 [SMILES: CC1(C(=O)NCc2cc3c(F)c(Cl)c(OCc4cscn4)cc3[nH]2)CC1] | N-((5-chloro-4-fluoro-6-(thiazol-4- ylmethoxy)-1H-indol-2-yl)methyl)-1- methylcyclopropane-1-carboxamide |
| 18 | 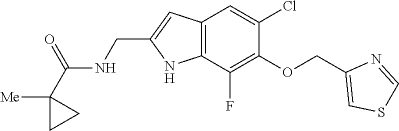 [SMILES: CC1(C(=O)NCc2cc3cc(Cl)c(OCc4cscn4)c(F)c3[nH]2)CC1] | N-((5-chloro-7-fluoro-6-(thiazol-4- ylmethoxy)-1H-indol-2-yl)methyl)-1- methylcyclopropane-1-carboxamide |
| 19 | 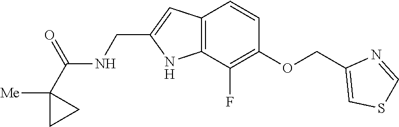 [SMILES: CC1(C(=O)NCc2cc3ccc(OCc4cscn4)c(F)c3[nH]2)CC1] | N-((7-fluoro-6-(thiazol-4-ylmethoxy)- 1H-indol-2-yl)methyl)-1- methylcyclopropane-1-carboxamide |
| 20 | 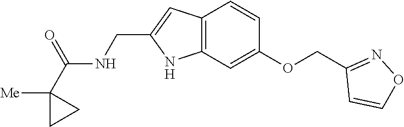 [SMILES: CC1(C(=O)NCc2cc3ccc(OCc4ccon4)cc3[nH]2)CC1] | N-((6-(isoxazol-3-ylmethoxy)-1H- indol-2-yl)methyl)-1- methylcyclopropane-1-carboxamide |
| 21 | 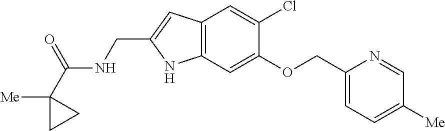 [SMILES: Cc1ccc(COc2cc3[nH]c(CNC(=O)C4(C)CC4)cc3cc2Cl)nc1] | N-((5-chloro-6-((5-methylpyridin-2- yl)methoxy)-1H-indol-2-yl)methyl)-1- methylcyclopropane-1-carboxamide |
| 22 | 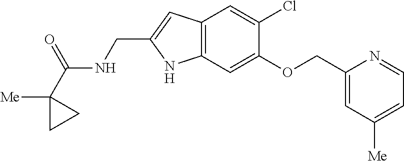 [SMILES: Cc1ccnc(COc2cc3[nH]c(CNC(=O)C4(C)CC4)cc3cc2Cl)c1] | N-((5-chloro-6-((4-methylpyridin-2- yl)methoxy)-1H-indol-2-yl)methyl)-1- methylcyclopropane-1-carboxamide |
| 23 | 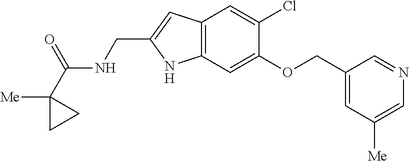 [SMILES: Cc1cncc(COc2cc3[nH]c(CNC(=O)C4(C)CC4)cc3cc2Cl)c1] | N-((5-chloro-6-((5-methylpyridin-3- yl)methoxy)-1H-indol-2-yl)methyl)-1- methylcyclopropane-1-carboxamide |
| 24 | 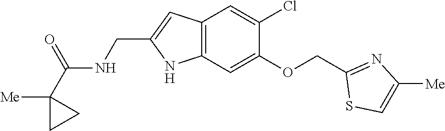 [SMILES: Cc1csc(COc2cc3[nH]c(CNC(=O)C4(C)CC4)cc3cc2Cl)n1] | N-((5-chloro-6-((4-methylthiazol-2- yl)methoxy)-1H-indol-2-yl)methyl)-1- methylcyclopropane-1-carboxamide |
| 25 | 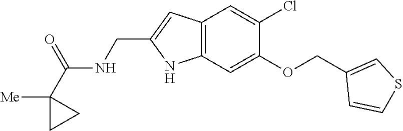 [SMILES: CC1(C(=O)NCc2cc3cc(Cl)c(OCc4ccsc4)cc3[nH]2)CC1] | N-((5-chloro-6-(thiophen-3- ylmethoxy)-1H-indol-2-yl)methyl)-1- methylcyclopropane-1-carboxamide |
| 26 | 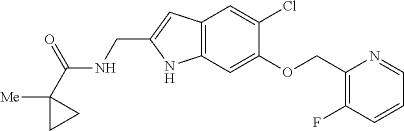 [SMILES: CC1(C(=O)NCc2cc3cc(Cl)c(OCc4ncccc4F)cc3[nH]2)CC1] | N-((5-chloro-6-((3-fluoropyridin-2- yl)methoxy)-1H-indol-2-yl)methyl)-1- methylcyclopropane-1-carboxamide |
| 27 | 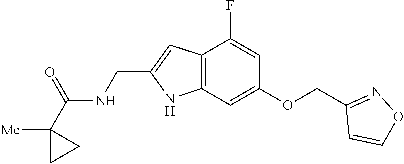 [SMILES: CC1(C(=O)NCc2cc3c(F)cc(OCc4ccon4)cc3[nH]2)CC1] | N-((4-fluoro-6-(isoxazol-3-ylmethoxy)- 1H-indol-2-yl)methyl)-1- methylcyclopropane-1-carboxamide |
| 28 | 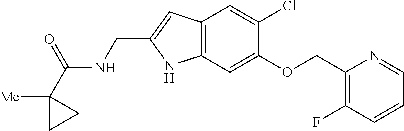 [SMILES: CC1(C(=O)NCc2cc3cc(Cl)c(OCc4ncccc4F)cc3[nH]2)CC1] | N-((5-chloro-6-((5-fluoropyridin-2- yl)methoxy)-1H-indol-2-yl)methyl)-1- methylcyclopropane-1-carboxamide |
| 29 | 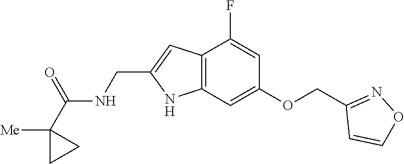 [SMILES: CC1(C(=O)NCc2cc3c(F)cc(OCc4ccon4)cc3[nH]2)CC1] | N-((4-fluoro-6-(thiazol-4-ylmethoxy)- 1H-indol-2-yl)methyl)-1- methylcyclopropane-1-carboxamide |
| 30 | 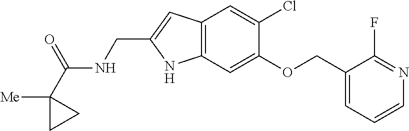 [SMILES: CC1(C(=O)NCc2cc3cc(Cl)c(OCc4cccnc4F)cc3[nH]2)CC1] | N-((5-chloro-6-((2-fluoropyridin-3- yl)methoxy)-1H-indol-2-yl)methyl)-1- methylcyclopropane-1-carboxamide |
| 31 | 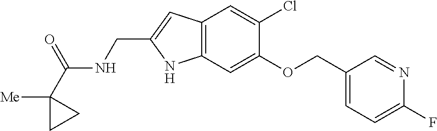 [SMILES: CC1(C(=O)NCc2cc3cc(Cl)c(OCc4ccc(F)nc4)cc3[nH]2)CC1] | N-((5-chloro-6-((6-fluoropyridin-3- yl)methoxy)-1H-indol-2-yl)methyl)-1- methylcyclopropane-1-carboxamide |
| 32 | 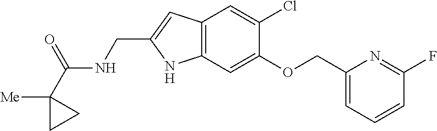 [SMILES: CC1(C(=O)NCc2cc3cc(Cl)c(OCc4cccc(F)n4)cc3[nH]2)CC1] | N-((5-chloro-6-((6-fluoropyridin-2- yl)methoxy)-1H-indol-2-yl)methyl)-1- methylcyclopropane-1-carboxamide |
| 33 | 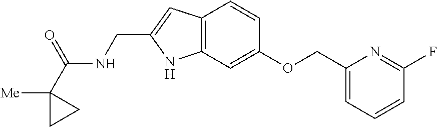 [SMILES: CC1(C(=O)NCc2cc3ccc(OCc4cccc(F)n4)cc3[nH]2)CC1] | N-((6-((6-fluoropyridin-2-yl)methoxy)- 1H-indol-2-yl)methyl)-1- methylcyclopropane-1-carboxamide |
| 34 | 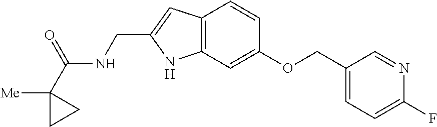 [SMILES: CC1(C(=O)NCc2cc3ccc(OCc4ccc(F)nc4)cc3[nH]2)CC1] | N-((6-((6-fluoropyridin-3-yl)methoxy)- 1H-indol-2-yl)methyl)-1- methylcyclopropane-1-carboxamide |
| 35 | 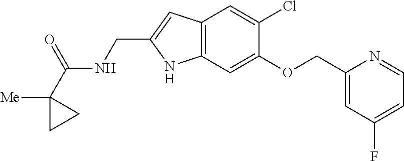 [SMILES: CC1(C(=O)NCc2cc3cc(Cl)c(OCc4cc(F)ccn4)cc3[nH]2)CC1] | N-((5-chloro-6-((4-fluoropyridin-2- yl)methoxy)-1H-indol-2-yl)methyl)-1- methylcyclopropane-1-carboxamide |
| 36 | 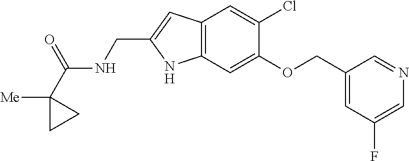 [SMILES: CC1(C(=O)NCc2cc3cc(Cl)c(OCc4cncc(F)c4)cc3[nH]2)CC1] | N-((5-chloro-6-((5-fluoropyridin-3- yl)methoxy)-1H-indol-2-yl)methyl)-1- methylcyclopropane-1-carboxamide |
| 37 | 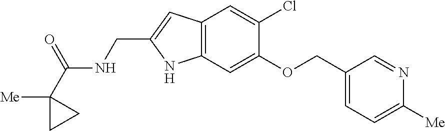 [SMILES: Cc1ccc(COc2cc3[nH]c(CNC(=O)C4(C)CC4)cc3cc2Cl)cn1] | N-((5-chloro-6-((6-methylpyridin-3- yl)methoxy)-1H-indol-2-yl)methyl)-1- methylcyclopropane-1-carboxamide |
| 38 | 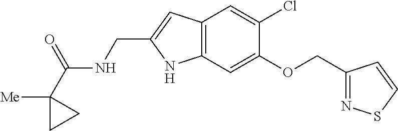 [SMILES: CC1(C(=O)NCc2cc3cc(Cl)c(OCc4ccsn4)cc3[nH]2)CC1] | N-((5-chloro-6-(isothiazol-3- ylmethoxy)-1H-indol-2-yl)methyl)-1- methylcyclopropane-1-carboxamide |
| 39 | 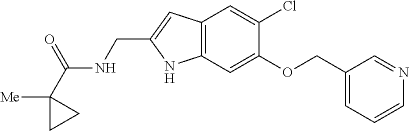 [SMILES: CC1(C(=O)NCc2cc3cc(Cl)c(OCc4cccnc4)cc3[nH]2)CC1] | N-((5-chloro-6-(pyridin-3-ylmethoxy)- 1H-indol-2-yl)methyl)-1- methylcyclopropane-1-carboxamide |
| 40 | 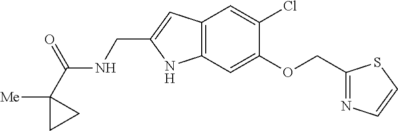 [SMILES: CC1(C(=O)NCc2cc3cc(Cl)c(OCc4nccs4)cc3[nH]2)CC1] | N-((5-chloro-6-(thiazol-2-ylmethoxy)- 1H-indol-2-yl)methyl)-1- methylcyclopropane-1-carboxamide |
| 41 | 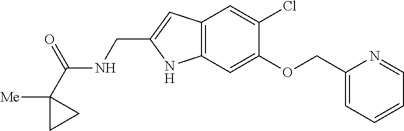 [SMILES: CC1(C(=O)NCc2cc3cc(Cl)c(OCc4ccccn4)cc3[nH]2)CC1] | N-((5-chloro-6-(pyridin-2-ylmethoxy)- 1H-indol-2-yl)methyl)-1- methylcyclopropane-1-carboxamide |
| 42 | 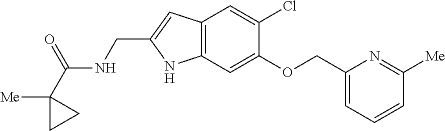 [SMILES: Cc1cccc(COc2cc3[nH]c(CNC(=O)C4(C)CC4)cc3cc2Cl)n1] | N-((5-chloro-6-((6-methylpyridin-2- yl)methoxy)-1H-indol-2-yl)methyl)-1- methylcyclopropane-1-carboxamide |
| 43 | 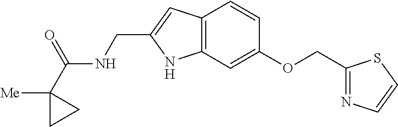 [SMILES: CC1(C(=O)NCc2cc3ccc(OCc4nccs4)cc3[nH]2)CC1] | 1-methyl-N-((6-(thiazol-2-ylmethoxy)- 1H-indol-2-yl)methyl)cyclopropane-1- carboxamide |
| 44 | 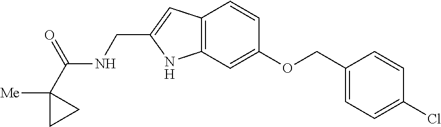 [SMILES: CC1(C(=O)NCc2cc3ccc(OCc4ccc(Cl)cc4)cc3[nH]2)CC1] | N-((6-((4-chlorobenzyl)oxy)-1H-indol- 2-yl)methyl)-1-methylcyclopropane-1- carboxamide |
| 45 | 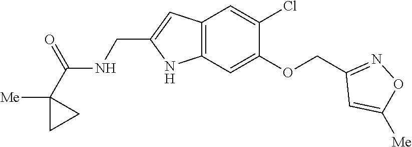 [SMILES: Cc1cc(COc2cc3[nH]c(CNC(=O)C4(C)CC4)cc3cc2Cl)no1] | N-((5-chloro-6-((5-methylisoxazol-3- yl)methoxy)-1H-indol-2-yl)methyl)-1- methylcyclopropane-1-carboxamide |
| 46 | 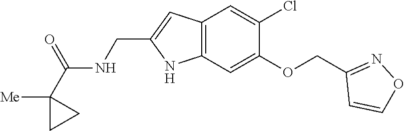 [SMILES: CC1(C(=O)NCc2cc3cc(Cl)c(OCc4ccon4)cc3[nH]2)CC1] | N-((5-chloro-6-(isoxazol-3-ylmethoxy)- 1H-indol-2-yl)methyl)-1- methylcyclopropane-1-carboxamide |
| 47 | 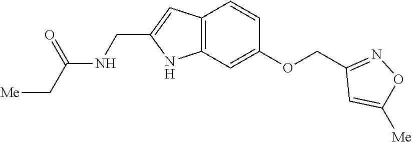 [SMILES: CCC(=O)NCc1cc2ccc(OCc3cc(C)on3)cc2[nH]1] | N-((6-((5-methylisoxazol-3- yl)methoxy)-1H-indol-2- yl)methyl)propionamide |
| 48 | 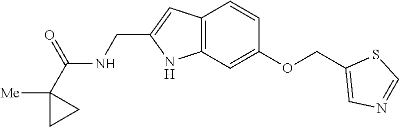 [SMILES: CC1(C(=O)NCc2cc3ccc(OCc4cncs4)cc3[nH]2)CC1] | 1-methyl-N-((6-(thiazol-5-ylmethoxy)- 1H-indol-2-yl)methyl)cyclopropane-1- carboxamide |
| 49 | 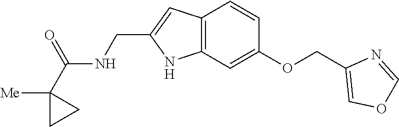 [SMILES: CC1(C(=O)NCc2cc3ccc(OCc4cocn4)cc3[nH]2)CC1] | 1-methyl-N-((6-(oxazol-4-ylmethoxy)- 1H-indol-2-yl)methyl)cyclopropane-1- carboxamide |
| 50 | 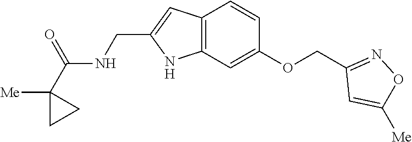 [SMILES: Cc1cc(COc2ccc3cc(CNC(=O)C4(C)CC4)[nH]c3c2)no1] | 1-methyl-N-((6-((5-methylisoxazol-3- yl)methoxy)-1H-indol-2- yl)methyl)cyclopropane-1-carboxamide |
| 51 | 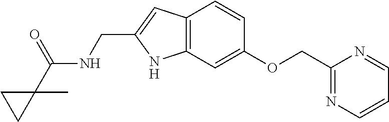 [SMILES: CC1(C(=O)NCc2cc3ccc(OCc4ncccn4)cc3[nH]2)CC1] | 1-methyl-N-((6-(pyrimidin-2- ylmethoxy)-1H-indol-2- yl)methyl)cyclopropane-1-carboxamide |
| 52 | 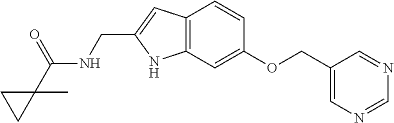 [SMILES: CC1(C(=O)NCc2cc3ccc(OCc4cncnc4)cc3[nH]2)CC1] | 1-methyl-N-((6-(pyrimidin-5- ylmethoxy)-1H-indol-2- yl)methyl)cyclopropane-1-carboxamide |
| 53 | 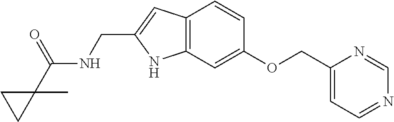 [SMILES: CC1(C(=O)NCc2cc3ccc(OCc4ccncn4)cc3[nH]2)CC1] | 1-methyl-N-((6-(pyrimidin-4- ylmethoxy)-1H-indol-2- yl)methyl)cyclopropane-1-carboxamide |
| 54 | 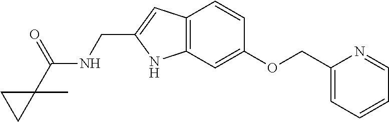 [SMILES: CC1(C(=O)NCc2cc3ccc(OCc4ccccn4)cc3[nH]2)CC1] | 1-methyl-N-((6-(pyridin-2-ylmethoxy)- 1H-indol-2-yl)methyl)cyclopropane-1- carboxamide |
| 55 | 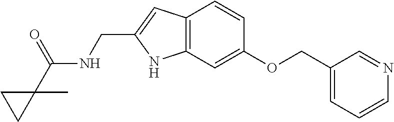 [SMILES: CC1(C(=O)NCc2cc3ccc(OCc4cccnc4)cc3[nH]2)CC1] | 1-methyl-N-((6-(pyridin-3-ylmethoxy)- 1H-indol-2-yl)methyl)cyclopropane-1- carboxamide |
| 56 | 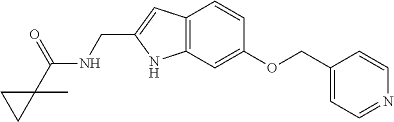 [SMILES: CC1(C(=O)NCc2cc3ccc(OCc4ccncc4)cc3[nH]2)CC1] | 1-methyl-N-((6-(pyridin-4-ylmethoxy)- 1H-indol-2-yl)methyl)cyclopropane-1- carboxamide |
| 57 | 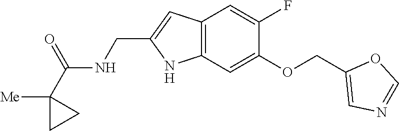 [SMILES: CC1(C(=O)NCc2cc3cc(F)c(OCc4cnco4)cc3[nH]2)CC1] | N-((5-fluoro-6-(oxazol-5-ylmethoxy)- 1H-indol-2-yl) methyl)-1- methylcyclopropane-1-carboxamide |
| 58 | 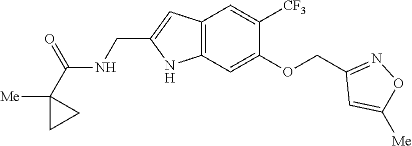 [SMILES: Cc1cc(COc2cc3[nH]c(CNC(=O)C4(C)CC4)cc3cc2C(F)(F)F)no1] | 1-methyl-N-((6-((5-methylisoxazol-3- yl)methoxy)-5-(trifluoromethyl)-1H- indol-2- yl)methyl)cyclopropanecarboxamide |
| 59 | 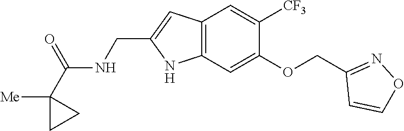 [SMILES: CC1(C(=O)NCc2cc3cc(C(F)(F)F)c(OCc4ccon4)cc3[nH]2)CC1] | N-((6-(isoxazol-3-ylmethoxy)-5- (trifluoromethyl)-1H-indol-2- yl)methyl)-1-methylcyclopropane-1- carboxamide |
| 60 | 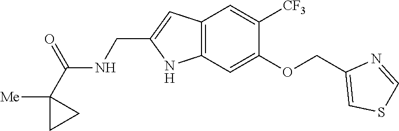 [SMILES: CC1(C(=O)NCc2cc3cc(C(F)(F)F)c(OCc4cscn4)cc3[nH]2)CC1] | 1-methyl-N-((6-(thiazol-4-ylmethoxy)- 5-(trifluoromethyl)-1H-indol-2- yl)methyl)cyclopropane-1-carboxamide |
| 61 | 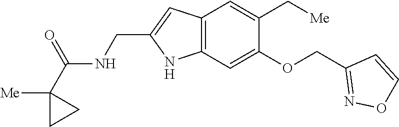 [SMILES: CCc1cc2cc(CNC(=O)C3(C)CC3)[nH]c2cc1OCc1ccon1] | N-((5-ethyl-6-(isoxazol-3-ylmethoxy)- 1H-indol-2-yl)methyl)-1- methylcyclopropane-1-carboxamide |
| 62 | 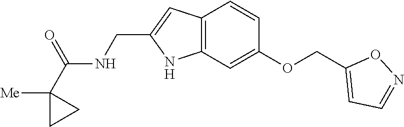 [SMILES: CC1(C(=O)NCc2cc3ccc(OCc4ccno4)cc3[nH]2)CC1] | N-((6-(isoxazol-5-ylmethoxy)-1H- indol-2-yl)methyl)-1- methylcyclopropanecarboxamide |
| 63 | 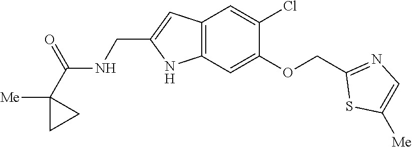 [SMILES: Cc1cnc(COc2cc3[nH]c(CNC(=O)C4(C)CC4)cc3cc2Cl)s1] | N-((5-chloro-6-((5-methylthiazol-2- yl)methoxy)-1H-indol-2-yl)methyl)-1- methylcyclopropane-1-carboxamide |
| 64 | 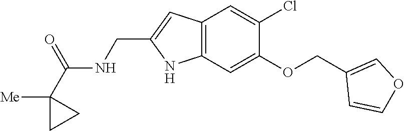 [SMILES: CC1(C(=O)NCc2cc3cc(Cl)c(OCc4ccoc4)cc3[nH]2)CC1] | N-((5-chloro-6-(furan-3-ylmethoxy)- 1H-indol-2-yl)methyl)-1- methylcyclopropane-1-carboxamide |
| 65 | 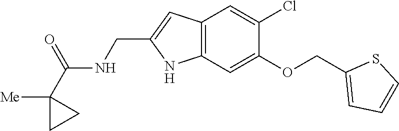 [SMILES: CC1(C(=O)NCc2cc3cc(Cl)c(OCc4cccs4)cc3[nH]2)CC1] | N-((5-chloro-6-(thiophen-2- ylmethoxy)-1H-indol-2-yl)methyl)-1- methylcyclopropane-1-carboxamide |
| 66 | 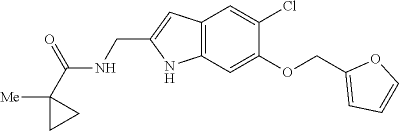 [SMILES: CC1(C(=O)NCc2cc3cc(Cl)c(OCc4ccco4)cc3[nH]2)CC1] | N-((5-chloro-6-(furan-2-ylmethoxy)- 1H-indol-2-yl)methyl)-1- methylcyclopropane-1-carboxamide |
| 67 | 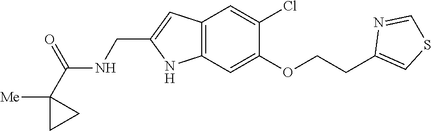 [SMILES: CC1(C(=O)NCc2cc3cc(Cl)c(OCCc4cscn4)cc3[nH]2)CC1] | N-((5-chloro-6-(2-(thiazol-4- yl)ethoxy)-1H-indol-2-yl)methyl)-1- methylcyclopropane-1-carboxamide |
| 68 | 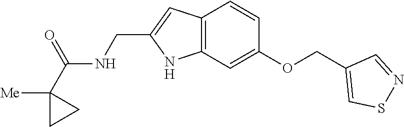 [SMILES: CC1(C(=O)NCc2cc3ccc(OCc4cnsc4)cc3[nH]2)CC1] | N-((6-(isothiazol-4-ylmethoxy)-1H- indol-2-yl)methyl)-1- methylcyclopropane-1-carboxamide |
| 69 | 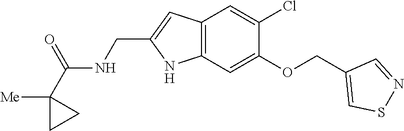 [SMILES: CC1(C(=O)NCc2cc3cc(Cl)c(OCc4cnsc4)cc3[nH]2)CC1] | N-((5-chloro-6-(isothiazol-4- ylmethoxy)-1H-indol-2-yl)methyl)-1- methylcyclopropane-1-carboxamide |
| 70 | 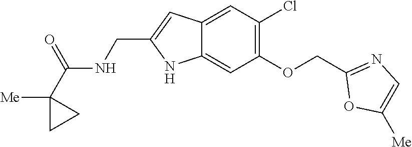 [SMILES: Cc1cnc(COc2cc3[nH]c(CNC(=O)C4(C)CC4)cc3cc2Cl)o1] | N-((5-chloro-6-((5-methyloxazol-2- yl)methoxy)-1H-indol-2-yl)methyl)-1- methylcyclopropane-1-carboxamide |
| 71 | 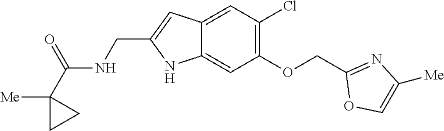 [SMILES: Cc1coc(COc2cc3[nH]c(CNC(=O)C4(C)CC4)cc3cc2Cl)n1] | N-((5-chloro-6-((4-methyloxazol-2- yl)methoxy)-1H-indol-2-yl)methyl)-1- methylcyclopropane-1-carboxamide |
| 72 | 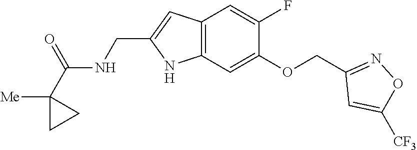 [SMILES: CC1(C(=O)NCc2cc3cc(F)c(OCc4cc(C(F)(F)F)on4)cc3[nH]2)CC1] | N-((5-fluoro-6-((5- (trifluoromethyl)isoxazol-3- yl)methoxy)-1H-indol-2-yl)methyl)-1- methylcyclopropane-1-carboxamide |
| 73 | 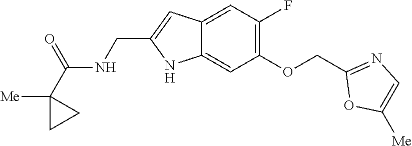 [SMILES: Cc1cnc(COc2cc3[nH]c(CNC(=O)C4(C)CC4)cc3cc2F)o1] | N-((5-fluoro-6-((5-methyloxazol-2- yl)methoxy)-1H-indol-2-yl)methyl)-1- methylcyclopropane-1-carboxamide |
| 74 | 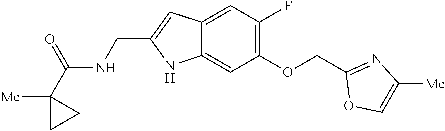 [SMILES: Cc1coc(COc2cc3[nH]c(CNC(=O)C4(C)CC4)cc3cc2F)n1] | N-((5-fluoro-6-((5-methyloxazol-2- yl)methoxy)-1H-indol-2-yl)methyl)-1- methylcyclopropane-1-carboxamide |
| 75 | 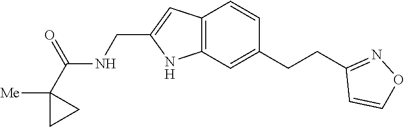 [SMILES: CC1(C(=O)NCc2cc3ccc(CCc4ccon4)cc3[nH]2)CC1] | N-((6-(2-(isoxazol-3-yl)ethyl)-1H- indol-2-yl)methyl)-1- methylcyclopropanecarboxamide |
| 76 | 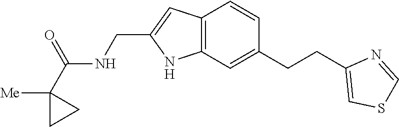 [SMILES: CC1(C(=O)NCc2cc3ccc(CCc4cscn4)cc3[nH]2)CC1] | 1-methyl-N-((6-(2-(thiazol-4-yl)ethyl)- 1H-indol-2-yl)methyl)cyclopropane-1- carboxamide |
| 77 | 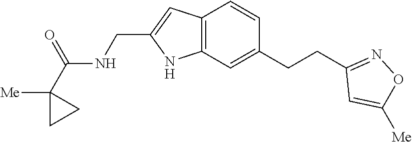 [SMILES: Cc1cc(CCc2ccc3cc(CNC(=O)C4(C)CC4)[nH]c3c2)no1] | 1-methyl-N-((6-(2-(5-methylisoxazol- 3-yl)ethyl)-1H-indol-2- yl)methyl)cyclopropane-1-carboxamide |
| 78 | 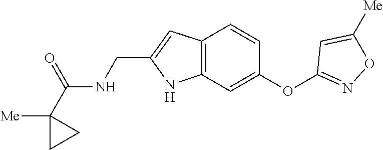 [SMILES: Cc1cc(Oc2ccc3cc(CNC(=O)C4(C)CC4)[nH]c3c2)no1] | 1-methyl-N-((6-((5-methylisoxazol-3- yl)oxy)-1H-indol-2- yl)methyl)cyclopropanecarboxamide |
| 79 | 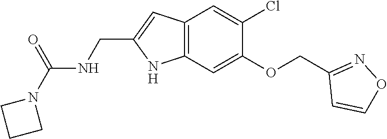 [SMILES: O=C(NCc1cc2cc(Cl)c(OCc3ccon3)cc2[nH]1)N1CCC1] | N-((5-chloro-6-(isoxazol-3-ylmethoxy)- 1H-indol-2-yl)methyl)azetidine-1- carboxamide |
| 80 | 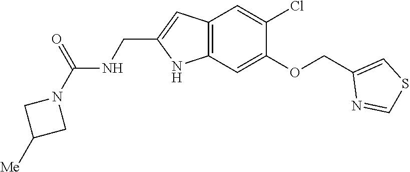 [SMILES: CC1CN(C(=O)NCc2cc3cc(Cl)c(OCc4cscn4)cc3[nH]2)C1] | N-((5-chloro-6-(thiazol-4-ylmethoxy)- 1H-indol-2-yl)methyl)-3- methylazetidine-1-carboxamide |
| 81 | 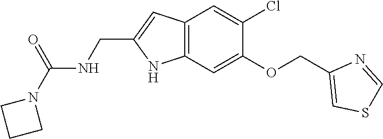 [SMILES: O=C(NCc1cc2cc(Cl)c(OCc3cscn3)cc2[nH]1)N1CCC1] | N-((5-chloro-6-(thiazol-4-ylmethoxy)- 1H-indol-2-yl)methyl)azetidine-1- carboxamide |
| 82 | 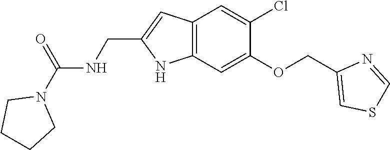 [SMILES: O=C(NCc1cc2cc(Cl)c(OCc3cscn3)cc2[nH]1)N1CCCC1] | N-((5-chloro-6-(thiazol-4-ylmethoxy)- 1H-indol-2-yl)methyl)pyrrolidine-1- carboxamide |
| 83 | 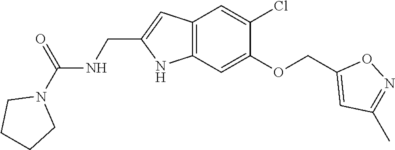 [SMILES: Cc1cc(COc2cc3[nH]c(CNC(=O)N4CCCC4)cc3cc2Cl)on1] | N-((5-chloro-6-((3-methylisoxazol-5- yl)methoxy)-1H-indol-2- yl)methyl)pyrrolidine-1-carboxamide |
| 84 | 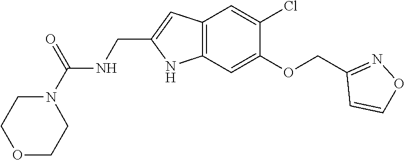 [SMILES: O=C(NCc1cc2cc(Cl)c(OCc3ccon3)cc2[nH]1)N1CCOCC1] | N-((5-chloro-6-(isoxazol-3-ylmethoxy)- 1H-indol-2-yl)methyl)morpholine-4- carboxamide |
| 85 | 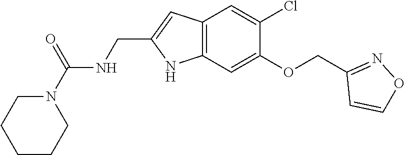 [SMILES: O=C(NCc1cc2cc(Cl)c(OCc3ccon3)cc2[nH]1)N1CCCCC1] | N-((5-chloro-6-(isoxazol-3-ylmethoxy)- 1H-indol-2-yl)methyl)piperidine-1- carboxamide |
| 86 | 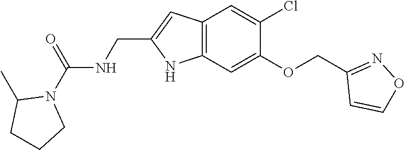 [SMILES: CC1CCCN1C(=O)NCc1cc2cc(Cl)c(OCc3ccon3)cc2[nH]1] | N-((5-chloro-6-(isoxazol-3-ylmethoxy)- 1H-indol-2-yl)methyl)-2- methylpyrrolidine-1-carboxamide |
| 87 | 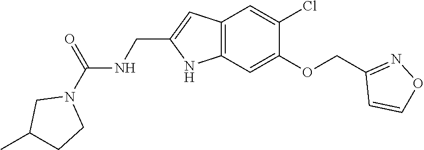 [SMILES: CC1CCN(C(=O)NCc2cc3cc(Cl)c(OCc4ccon4)cc3[nH]2)C1] | N-((5-chloro-6-(isoxazol-3-ylmethoxy)- 1H-indol-2-yl)methyl)-3- methylpyrrolidine-1-carboxamide |
| 88 | 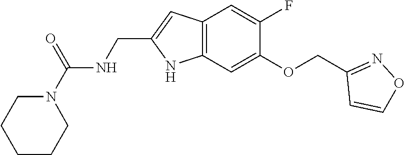 [SMILES: O=C(NCc1cc2cc(F)c(OCc3ccon3)cc2[nH]1)N1CCCCC1] | N-((5-fluoro-6-(isoxazol-3-ylmethoxy)- 1H-indol-2-yl)methyl)piperidine-1- carboxamide |
| 89 | 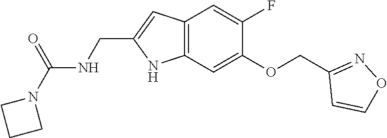 [SMILES: O=C(NCc1cc2cc(F)c(OCc3ccon3)cc2[nH]1)N1CCC1] | N-((5-fluoro-6-(isoxazol-3-ylmethoxy)- 1H-indol-2-yl)methyl)azetidine-1- carboxamide |
| 90 | 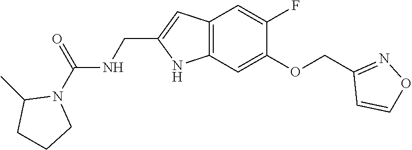 [SMILES: CC1CCCN1C(=O)NCc1cc2cc(F)c(OCc3ccon3)cc2[nH]1] | N-((5-fluoro-6-(isoxazol-3-ylmethoxy)- 1H-indol-2-yl)methyl)-2- methylpyrrolidine-1-carboxamide |
| 91 | 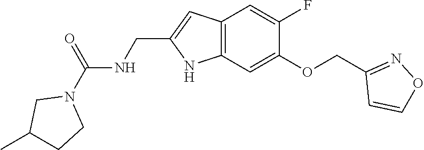 [SMILES: CC1CCN(C(=O)NCc2cc3cc(F)c(OCc4ccon4)cc3[nH]2)C1] | N-((5-fluoro-6-(isoxazol-3-ylmethoxy)- 1H-indol-2-yl)methyl)-3- methylpyrrolidine-1-carboxamide |
| 92 | 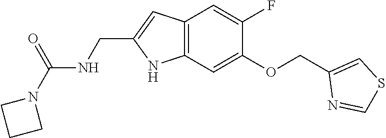 [SMILES: O=C(NCc1cc2cc(F)c(OCc3cscn3)cc2[nH]1)N1CCC1] | N-((5-fluoro-6-(thiazol-4-ylmethoxy)- 1H-indol-2-yl)methyl)azetidine-1- carboxamide |
| 93 | 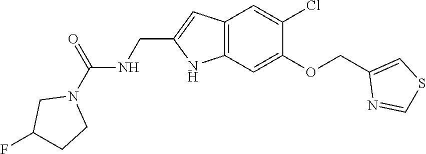 [SMILES: O=C(NCc1cc2cc(Cl)c(OCc3cscn3)cc2[nH]1)N1CCC(F)C1] | N-((5-chloro-6-(thiazol-4-ylmethoxy)- 1H-indol-2-yl)methyl)-3- fluoropyrrolidine-1-carboxamide |
| 94 | 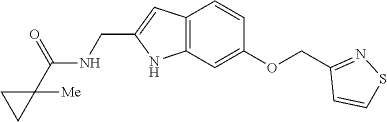 [SMILES: CC1(C(=O)NCc2cc3ccc(OCc4ccsn4)cc3[nH]2)CC1] | N-((6-(isothiazol-3-ylmethoxy)-1H- indol-2-yl)methyl)-1- methylcyclopropanecarboxamide |
| 95 | 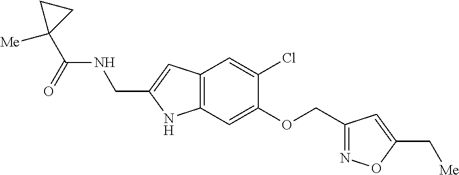 [SMILES: CCc1cc(COc2cc3[nH]c(CNC(=O)C4(C)CC4)cc3cc2Cl)no1] | (N-((5-chloro-6-((5-ethylisoxazol-3- yl)methoxy)-1H-indol-2-yl)methyl)-1- methylcyclopropanecarboxamide |
| 96 | 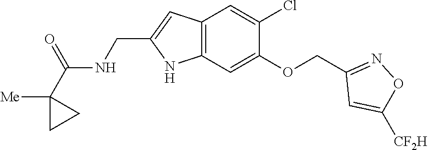 [SMILES: CC1(C(=O)NCc2cc3cc(Cl)c(OCc4cc(C(F)F)on4)cc3[nH]2)CC1] | N-((5-chloro-6-((5- (difluoromethyl)isoxazol-3- yl)methoxy)-1H-indol-2-yl)methyl)-1- methylcyclopropane-1-carboxamide |
| 97 | 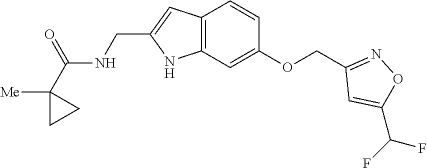 [SMILES: CC1(C(=O)NCc2cc3ccc(OCc4cc(C(F)F)on4)cc3[nH]2)CC1] | N-((6-((5-(difluoromethyl)isoxazol-3- yl)methoxy)-1H-indol-2-yl)methyl)-1- methylcyclopropane-1-carboxamide |
| 98 | 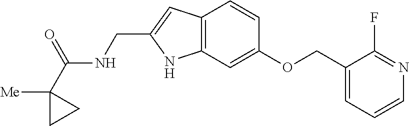 [SMILES: CC1(C(=O)NCc2cc3ccc(OCc4cccnc4F)cc3[nH]2)CC1] | N-((6-((2-fluoropyridin-3-yl)methoxy)- 1H-indol-2-yl)methyl)-1- methylcyclopropane-1-carboxamide formate |
| 99 | 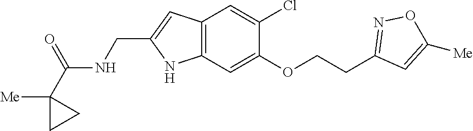 [SMILES: Cc1cc(CCOc2cc3[nH]c(CNC(=O)C4(C)CC4)cc3cc2Cl)no1] | N-((5-chloro-6-(2-(5-methylisoxazol-3- yl)ethoxy)-1H-indol-2-yl)methyl)-1- methylcyclopropane-1-carboxamide |
| 100 | 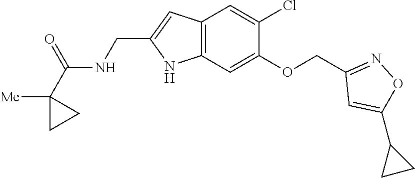 [SMILES: CC1(C(=O)NCc2cc3cc(Cl)c(OCc4cc(C5CC5)on4)cc3[nH]2)CC1] | N-((5-chloro-6-((5- cyclopropylisoxazol-3-yl)methoxy)- 1H-indol-2-yl)methyl)-1- methylcyclopropane-1-carboxamide |
| 101 | 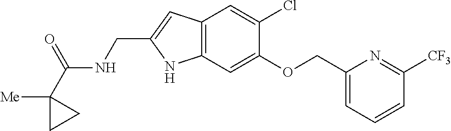 [SMILES: CC1(C(=O)NCc2cc3cc(Cl)c(OCc4cccc(C(F)(F)F)n4)cc3[nH]2)CC1] | N-((5-chloro-6-((6- (trifluoromethyl)pyridin-2- yl)methoxy)-1H-indol-2-yl)methyl)-1- methylcyclopropane-1-carboxamide |
| 102 | 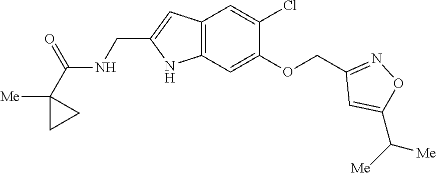 [SMILES: CC(C)c1cc(COc2cc3[nH]c(CNC(=O)C4(C)CC4)cc3cc2Cl)no1] | N-((5-chloro-6-((5-isopropylisoxazol-3- yl)methoxy)-1H-indol-2-yl)methyl)-1- methylcyclopropane-1-carboxamide |
| 103 | 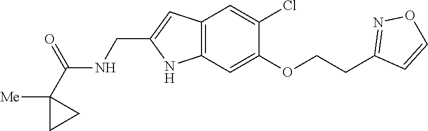 [SMILES: CC1(C(=O)NCc2cc3cc(Cl)c(OCCc4ccon4)cc3[nH]2)CC1] | N-((5-chloro-6-(2-(isoxazol-3- yl)ethoxy)-1H-indol-2-yl)methyl)-1- methylcyclopropane-1-carboxamide |
| 104 | 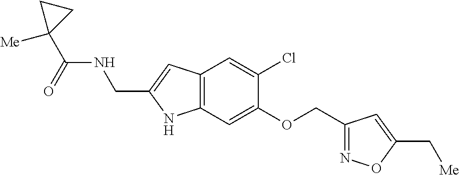 [SMILES: CCc1cc(COc2cc3[nH]c(CNC(=O)C4(C)CC4)cc3cc2Cl)no1] | N-((6-(isoxazol-3-ylmethoxy)-1H- indol-2-yl)methyl)pyrrolidine-1- carboxamide |
| 105 | 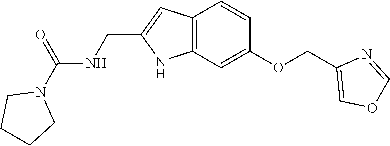 [SMILES: O=C(NCc1cc2ccc(OCc3cocn3)cc2[nH]1)N1CCCC1] | N-((6-(oxazol-4-ylmethoxy)-1H-indol- 2-yl)methyl)pyrrolidine-1-carboxamide |
| 106 | 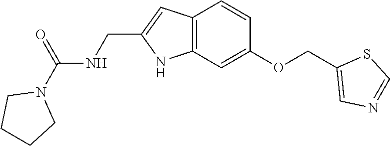 [SMILES: O=C(NCc1cc2ccc(OCc3cncs3)cc2[nH]1)N1CCCC1] | N-((6-(thiazol-5-ylmethoxy)-1H-indol- 2-yl)methyl)pyrrolidine-1-carboxamide |
| 107 | 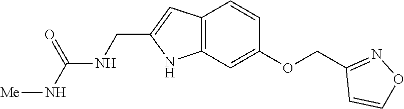 [SMILES: CNC(=O)NCc1cc2ccc(OCc3ccon3)cc2[nH]1] | 1-((6-(isoxazol-3-ylmethoxy)-1H-indol- 2-yl)methyl)-3-methylurea |
| 108 | 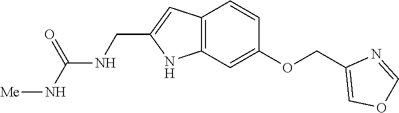 [SMILES: CNC(=O)NCc1cc2ccc(OCc3cocn3)cc2[nH]1] | 1-methyl-3-((6-(oxazol-4-ylmethoxy)- 1H-indol-2-yl)methyl)urea |
| 109 | 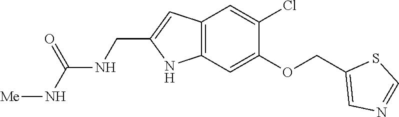 [SMILES: CNC(=O)NCc1cc2cc(Cl)c(OCc3cncs3)cc2[nH]1] | 1-((5-chloro-6-(thiazol-5-ylmethoxy)- 1H-indol-2-yl)methyl)-3-methylurea |
| 110 | 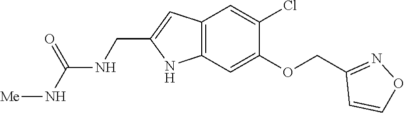 [SMILES: CNC(=O)NCc1cc2cc(Cl)c(OCc3ccon3)cc2[nH]1] | 1-((5-chloro-6-(isoxazol-3-ylmethoxy)- 1H-indol-2-yl)methyl)-3-methylurea |
| 111 | 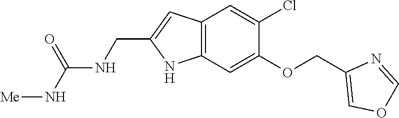 [SMILES: CNC(=O)NCc1cc2cc(Cl)c(OCc3cocn3)cc2[nH]1] | 1-((5-chloro-6-(oxazol-4-ylmethoxy)- 1H-indol-2-yl)methyl)-3-methylurea |
| 112 | 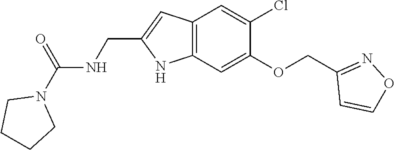 [SMILES: O=C(NCc1cc2cc(Cl)c(OCc3ccon3)cc2[nH]1)N1CCCC1] | N-((5-chloro-6-(isoxazol-3-ylmethoxy)- 1H-indol-2-yl)methyl)pyrrolidine-1- carboxamide |
| 113 | 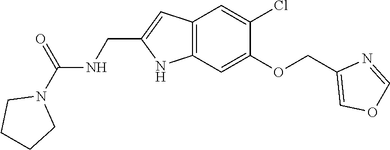 [SMILES: O=C(NCc1cc2cc(Cl)c(OCc3cocn3)cc2[nH]1)N1CCCC1] | N-((5-chloro-6-(oxazol-4-ylmethoxy)- 1H-indol-2-yl)methyl)pyrrolidine-1- carboxamide |
| 114 | 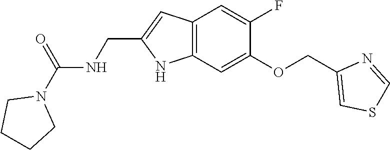 [SMILES: O=C(NCc1cc2cc(F)c(OCc3cscn3)cc2[nH]1)N1CCCC1] | N-((5-fluoro-6-(thiazol-4-ylmethoxy)- 1H-indol-2-yl)methyl)pyrrolidine-1- carboxamide |
| 115 | 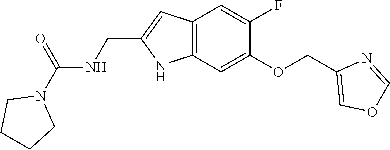 [SMILES: O=C(NCc1cc2cc(F)c(OCc3cocn3)cc2[nH]1)N1CCCC1] | N-((5-fluoro-6-(oxazol-4-ylmethoxy)- 1H-indol-2-yl)methyl)pyrrolidine-1- carboxamide |
| 116 | 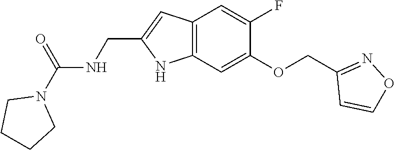 [SMILES: O=C(NCc1cc2cc(F)c(OCc3ccon3)cc2[nH]1)N1CCCC1] | N-((5-fluoro-6-(isoxazol-3-ylmethoxy)- 1H-indol-2-yl)methyl)pyrrolidine-1- carboxamide |
| 117 | 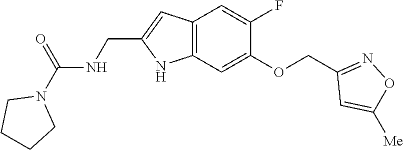 [SMILES: Cc1cc(COc2cc3[nH]c(CNC(=O)N4CCCC4)cc3cc2F)no1] | N-((5-fluoro-6-((5-methylisoxazol-3- yl)methoxy)-1H-indol-2- yl)methyl)pyrrolidine-1-carboxamide |
| 118 | 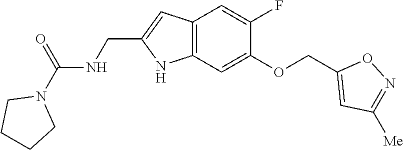 [SMILES: Cc1cc(COc2cc3[nH]c(CNC(=O)N4CCCC4)cc3cc2F)on1] | N-((5-fluoro-6-((3-methylisoxazol-5- yl)methoxy)-1H-indol-2- yl)methyl)pyrrolidine-1-carboxamide |
| 119 | 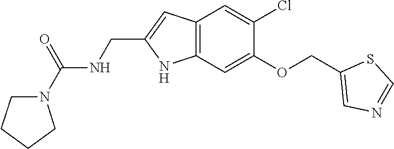 [SMILES: O=C(NCc1cc2cc(Cl)c(OCc3cncs3)cc2[nH]1)N1CCCC1] | N-((5-chloro-6-(thiazol-5-ylmethoxy)- 1H-indol-2-yl) methyl) pyrrolidine-1- carboxamide |
| 120 | 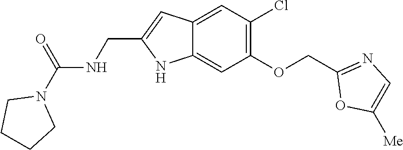 [SMILES: Cc1cnc(COc2cc3[nH]c(CNC(=O)N4CCCC4)cc3cc2Cl)o1] | N-((5-chloro-6-((5-methyloxazol-2- yl)methoxy)-1H-indol-2- yl)methyl)pyrrolidine-1-carboxamide |
| 121 | 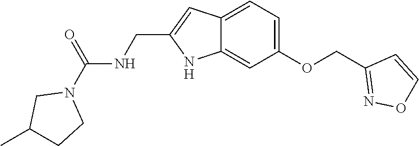 [SMILES: CC1CCN(C(=O)NCc2cc3ccc(OCc4ccon4)cc3[nH]2)C1] | N-((6-(isoxazol-3-ylmethoxy)-1H- indol-2-yl)methyl)-3- methylpyrrolidine-1-carboxamide |
| 122 | 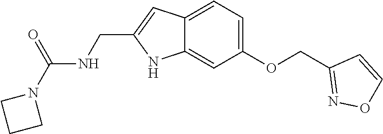 [SMILES: O=C(NCc1cc2ccc(OCc3ccon3)cc2[nH]1)N1CCC1] | N-((6-(isoxazol-3-ylmethoxy)-1H- indol-2-yl)methyl)azetidine-1- carboxamide |
| 123 | 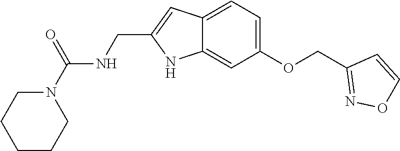 [SMILES: O=C(NCc1cc2ccc(OCc3ccon3)cc2[nH]1)N1CCCCC1] | N-((6-(isoxazol-3-ylmethoxy)-1H- indol-2-yl)methyl)piperidine-1- carboxamide |
| 124 | 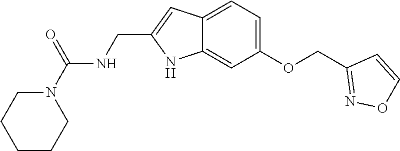 [SMILES: O=C(NCc1cc2ccc(OCc3ccon3)cc2[nH]1)N1CCCCC1] | N-((6-(isoxazol-3-ylmethoxy)-1H- indol-2-yl)methyl)-2- methylpyrrolidine-1-carboxamide |
| 125 | 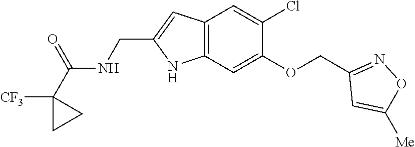 [SMILES: Cc1cc(COc2cc3[nH]c(CNC(=O)C4(C(F)(F)F)CC4)cc3cc2Cl)no1] | N-((5-chloro-6-((5-methylisoxazol-3- yl)methoxy)-1H-indol-2-yl)methyl)-1- (trifluoromethyl)cyclopropane-1- carboxamide |
| 126 | 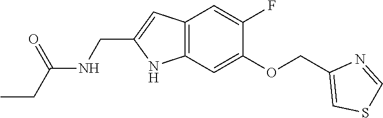 [SMILES: CCC(=O)NCc1cc2cc(F)c(OCc3cscn3)cc2[nH]1] | N-((5-fluoro-6-(thiazol-4-ylmethoxy)- 1H-indol-2-yl)methyl)propionamide |
| 127 | 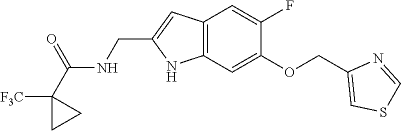 [SMILES: O=C(NCc1cc2cc(F)c(OCc3cscn3)cc2[nH]1)C1(C(F)(F)F)CC1] | N-((5-fluoro-6-(thiazol-4-ylmethoxy)- 1H-indol-2-yl)methyl)-1- (trifluoromethyl)cyclopropane-1- carboxamide |
| 128 | 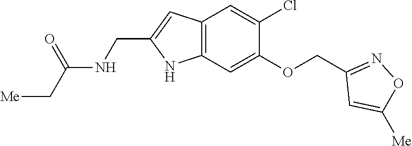 [SMILES: CCC(=O)NCc1cc2cc(Cl)c(OCc3cc(C)on3)cc2[nH]1] | N-((5-chloro-6-((5-methylisoxazol-3- yl)methoxy)-1H-indol-2- yl)methyl)propionamide |
| 129 | 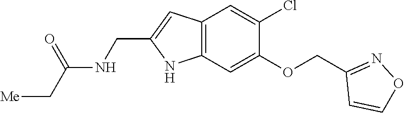 [SMILES: CCC(=O)NCc1cc2cc(Cl)c(OCc3ccon3)cc2[nH]1] | N-((5-chloro-6-(isoxazol-3-ylmethoxy)- 1H-indol-2-yl)methyl)propionamide |
| 130 | 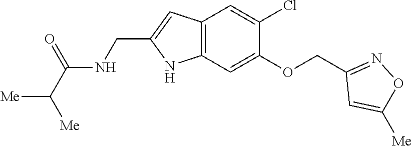 [SMILES: Cc1cc(COc2cc3[nH]c(CNC(=O)C(C)C)cc3cc2Cl)no1] | N-((5-chloro-6-((5-methylisoxazol-3- yl)methoxy)-1H-indol-2- yl)methyl)isobutyramide |
| 131 | 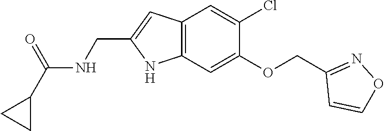 [SMILES: O=C(NCc1cc2cc(Cl)c(OCc3ccon3)cc2[nH]1)C1CC1] | N-((5-chloro-6-(isoxazol-3-ylmethoxy)- 1H-indol-2- yl)methyl)cyclopropanecarboxamide |
| 132 | 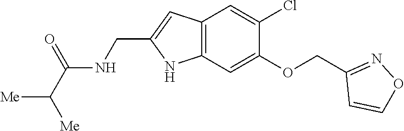 [SMILES: CC(C)C(=O)NCc1cc2cc(Cl)c(OCc3ccon3)cc2[nH]1] | N-((5-chloro-6-(isoxazol-3-ylmethoxy)- 1H-indol-2-yl)methyl)isobutyramide |
| 133 | 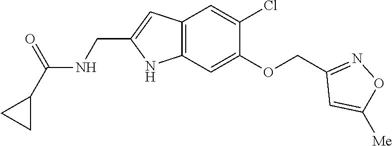 [SMILES: Cc1cc(COc2cc3[nH]c(CNC(=O)C4CC4)cc3cc2Cl)no1] | N-((5-chloro-6-((5-methylisoxazol-3- yl)methoxy)-1H-indol-2- yl)methyl)cyclopropanecarboxamide |
| 134 | 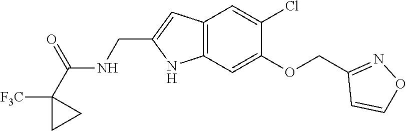 [SMILES: O=C(NCc1cc2cc(Cl)c(OCc3ccon3)cc2[nH]1)C1(C(F)(F)F)CC1] | N-((5-chloro-6-(isoxazol-3-ylmethoxy)- 1H-indol-2-yl)methyl)-1- (trifluoromethyl)cyclopropane-1- carboxamide |
| 135 | 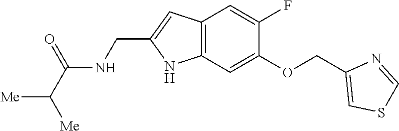 [SMILES: CC(C)C(=O)NCc1cc2cc(F)c(OCc3cscn3)cc2[nH]1] | N-((5-fluoro-6-(thiazol-4-ylmethoxy)- 1H-indol-2-yl)methyl)isobutyramide |
| 136 | 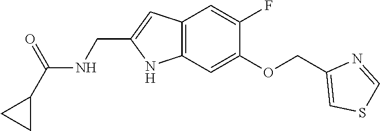 [SMILES: O=C(NCc1cc2cc(F)c(OCc3cscn3)cc2[nH]1)C1CC1] | N-((5-fluoro-6-(thiazol-4-ylmethoxy)- 1H-indol-2- yl)methyl)cyclopropanecarboxamide |
| 137 | 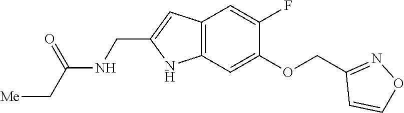 [SMILES: CCC(=O)NCc1cc2cc(F)c(OCc3ccon3)cc2[nH]1] | N-((5-fluoro-6-(isoxazol-3-ylmethoxy)- 1H-indol-2-yl)methyl)propionamide |
| 138 | 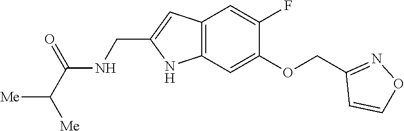 [SMILES: CC(C)C(=O)NCc1cc2cc(F)c(OCc3ccon3)cc2[nH]1] | N-((5-fluoro-6-(isoxazol-3-ylmethoxy)- 1H-indol-2-yl)methyl)isobutyramide |
| 139 | 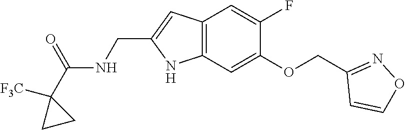 [SMILES: O=C(NCc1cc2cc(F)c(OCc3ccon3)cc2[nH]1)C1(C(F)(F)F)CC1] | N-((5-fluoro-6-(isoxazol-3-ylmethoxy)- 1H-indol-2-yl)methyl)-1- (trifluoromethyl)cyclopropane-1- carboxamide |
| 140 | 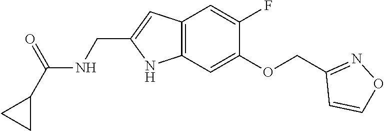 [SMILES: O=C(NCc1cc2cc(F)c(OCc3ccon3)cc2[nH]1)C1CC1] | N-((5-fluoro-6-(isoxazol-3-ylmethoxy)- 1H-indol-2- yl)methyl)cyclopropanecarboxamide |
| 141 | 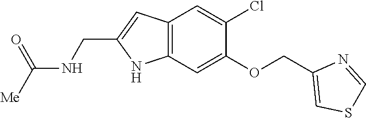 [SMILES: CC(=O)NCc1cc2cc(Cl)c(OCc3cscn3)cc2[nH]1] | N-((5-chloro-6-(thiazol-4-ylmethoxy)- 1H-indol-2-yl)methyl)acetamide |
| 142 | 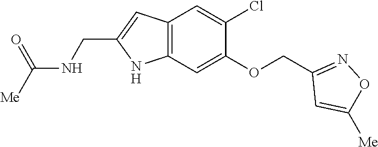 [SMILES: CC(=O)NCc1cc2cc(Cl)c(OCc3cc(C)on3)cc2[nH]1] | N-((5-chloro-6-((5-methylisoxazol-3- yl)methoxy)-1H-indol-2- yl)methyl)acetamide |
| 143 | 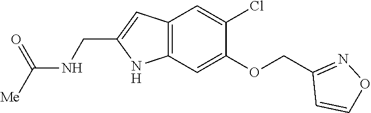 [SMILES: CC(=O)NCc1cc2cc(Cl)c(OCc3ccon3)cc2[nH]1] | N-((5-chloro-6-(isoxazol-3-ylmethoxy)- 1H-indol-2-yl)methyl)acetamide |
| 144 | 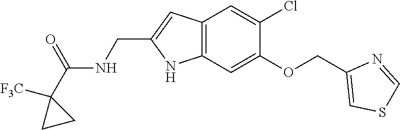 [SMILES: O=C(NCc1cc2cc(Cl)c(OCc3cscn3)cc2[nH]1)C1(C(F)(F)F)CC1] | N-((5-chloro-6-(thiazol-4-ylmethoxy)- 1H-indol-2-yl)methyl)-1- (trifluoromethyl)cyclopropane-1- carboxamide |
| 145 | 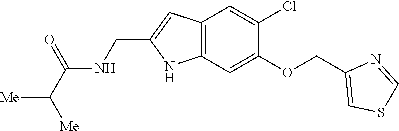 [SMILES: CC(C)C(=O)NCc1cc2cc(Cl)c(OCc3cscn3)cc2[nH]1] | N-((5-chloro-6-(thiazol-4-ylmethoxy)- 1H-indol-2-yl)methyl)isobutyramide |
| 146 | 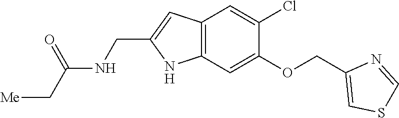 [SMILES: CCC(=O)NCc1cc2cc(Cl)c(OCc3cscn3)cc2[nH]1] | N-((5-chloro-6-(thiazol-4-ylmethoxy)- 1H-indol-2-yl)methyl)propionamide |
| 147 | 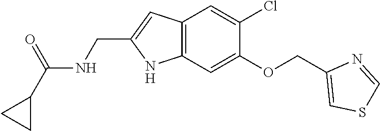 [SMILES: O=C(NCc1cc2cc(Cl)c(OCc3cscn3)cc2[nH]1)C1CC1] | N-((5-chloro-6-(thiazol-4-ylmethoxy)- 1H-indol-2- yl)methyl)cyclopropanecarboxamide |
| 148 | 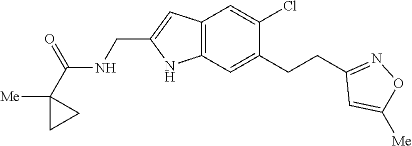 [SMILES: Cc1cc(CCc2cc3[nH]c(CNC(=O)C4(C)CC4)cc3cc2Cl)no1] | N-((5-chloro-6-(hydroxymethyl)-1H- indol-2-yl)methyl)-1- methylcyclopropanecarboxamide |
| 149 | 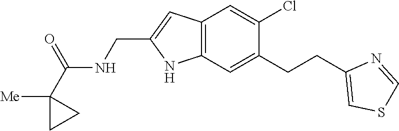 [SMILES: CC1(C(=O)NCc2cc3cc(Cl)c(CCc4cscn4)cc3[nH]2)CC1] | N-((5-chloro-6-(2-(thiazol-4-yl)ethyl)- 1H-indol-2-yl)methyl)-1- methylcyclopropane-1-carboxamide |
| 150 | 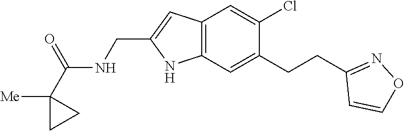 [SMILES: CC1(C(=O)NCc2cc3cc(Cl)c(CCc4ccon4)cc3[nH]2)CC1] | N-((5-chloro-6-(2-(isoxazol-3-yl)ethyl)- 1H-indol-2-yl)methyl)-1- methylcyclopropane-1-carboxamide |
| 151 | 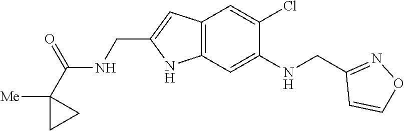 [SMILES: CC1(C(=O)NCc2cc3cc(Cl)c(NCc4ccon4)cc3[nH]2)CC1] | N-((5-chloro-6-((isoxazol-3- ylmethyl)amino)-1H-indol-2- yl)methyl)-1-methylcyclopropane-1- carboxamide |
| 152 | 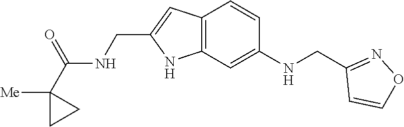 [SMILES: CC1(C(=O)NCc2cc3ccc(NCc4ccon4)cc3[nH]2)CC1] | N-((6-((isoxazol-3-ylmethyl)amino)- 1H-indol-2-yl)methyl)-1- methylcyclopropane-1-carboxamide |
| 153 | 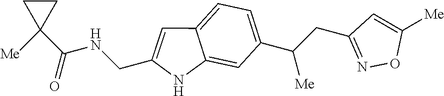 [SMILES: Cc1cc(CC(C)c2ccc3cc(CNC(=O)C4(C)CC4)[nH]c3c2)no1] | 1-methyl-N-((6-(1-(5-methylisoxazol- 3-yl) propan-2-yl)-1H-indol-2-yl) methyl) cyclopropane-1-carboxamide |
| 154 | 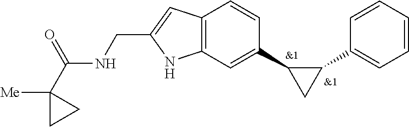 [SMILES: CC1(C(=O)NCc2cc3ccc([C@@H]4C[C@H]4c4ccccc4)cc3[nH]2)CC1] | trans-1-methyl-N-((6-(2- phenylcyclopropyl)-1H-indol-2- yl)methyl)cyclopropane-1-carboxamide |
| 155 | 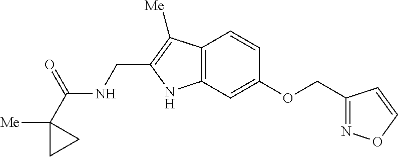 [SMILES: Cc1c(CNC(=O)C2(C)CC2)[nH]c2cc(OCc3ccon3)ccc12] | N-((6-(isoxazol-3-ylmethoxy)-3- methyl-1H-indol-2-yl)methyl)-1- methylcyclopropane-1-carboxamide |
| 156 | 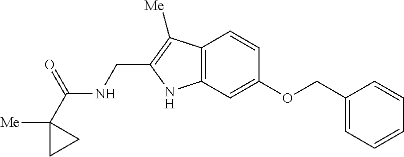 [SMILES: Cc1c(CNC(=O)C2(C)CC2)[nH]c2cc(OCc3ccccc3)ccc12] | N-((6-(benzyloxy)-3-methyl-1H-indol- 2-yl)methyl)-1-methylcyclopropane-1- carboxamide |
| 157 | 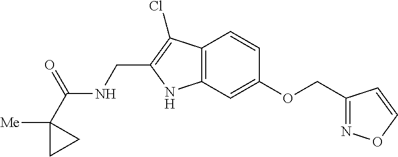 [SMILES: CC1(C(=O)NCc2[nH]c3cc(OCc4ccon4)ccc3c2Cl)CC1] | N-((3-chloro-6-(isoxazol-3-ylmethoxy)- 1H-indol-2-yl)methyl)-1- methylcyclopropane-1-carboxamide |
| 158 | 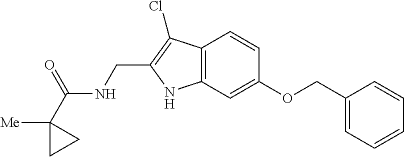 [SMILES: CC1(C(=O)NCc2[nH]c3cc(OCc4ccccc4)ccc3c2Cl)CC1] | N-((6-(benzyloxy)-3-chloro-1H-indol- 2-yl)methyl)-1-methylcyclopropane-1- carboxamide |
| 159 | 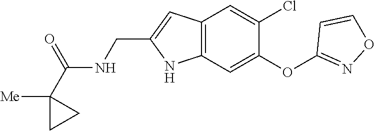 [SMILES: CC1(C(=O)NCc2cc3cc(Cl)c(Oc4ccon4)cc3[nH]2)CC1] | N-((5-chloro-6-(isoxazol-3-ylmethyl)- 1H-indol-2-yl)methyl)-1- methylcyclopropanecarboxamide |
| 160 | 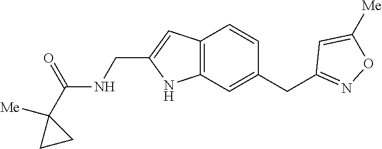 [SMILES: Cc1cc(Cc2ccc3cc(CNC(=O)C4(C)CC4)[nH]c3c2)no1] | 1-methyl-N-((6-((5-methylisoxazol-3- yl)methyl)-1H-indol-2- yl)methyl)cyclopropanecarboxamide |
| 161 | 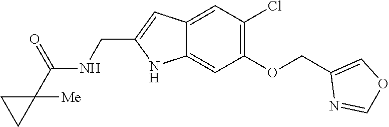 [SMILES: CC1(C(=O)NCc2cc3cc(Cl)c(OCc4cocn4)cc3[nH]2)CC1] | N-((5-chloro-6-(oxazol-4-ylmethoxy)- 1H-indol-2-yl)methyl)-1- methylcyclopropane-1-carboxamide |
| 162 | 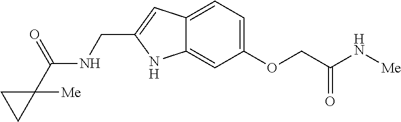 [SMILES: CNC(=O)COc1ccc2cc(CNC(=O)C3(C)CC3)[nH]c2c1] | 1-methyl-N-((6-(2-(methylamino)-2- oxoethoxy)-1H-indol-2- yl)methyl)cyclopropane-1-carboxamide |
| 163 | 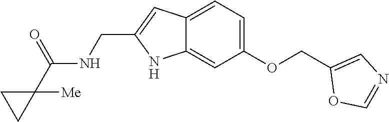 [SMILES: CC1(C(=O)NCc2cc3ccc(OCc4cnco4)cc3[nH]2)CC1] | 1-methyl-N-((6-(oxazol-5-ylmethoxy)- 1H-indol-2-yl)methyl)cyclopropane-1- carboxamide |
| 164 | 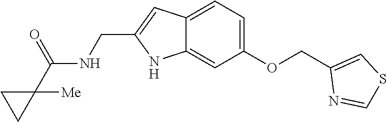 [SMILES: CC1(C(=O)NCc2cc3ccc(OCc4cscn4)cc3[nH]2)CC1] | 1-methyl-N-((6-(thiazol-4-ylmethoxy)- 1H-indol-2-yl)methyl)cyclopropane-1- carboxamide |
| 165 | 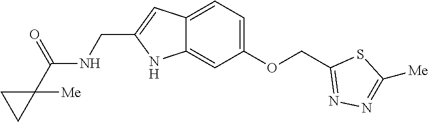 [SMILES: Cc1nnc(COc2ccc3cc(CNC(=O)C4(C)CC4)[nH]c3c2)s1] | 1-methyl-N-((6-((5-methyl-1,3,4- thiadiazol-2-yl)methoxy)-1H-indol-2- yl)methyl)cyclopropane-1-carboxamide |
| 166 | 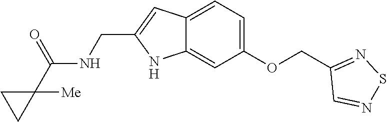 [SMILES: CC1(C(=O)NCc2cc3ccc(OCc4cnsn4)cc3[nH]2)CC1] | N-((6-((1,2,5-thiadiazol-3-yl)methoxy)- 1H-indol-2-yl)methyl)-1- methylcyclopropane-1-carboxamide |
| 167 | 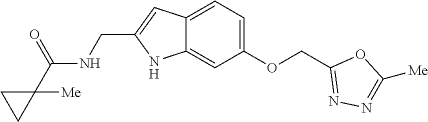 [SMILES: Cc1nnc(COc2ccc3cc(CNC(=O)C4(C)CC4)[nH]c3c2)o1] | 1-methyl-N-((6-((5-methyl-1,3,4- oxadiazol-2-yl)methoxy)-1H-indol-2- yl)methyl)cyclopropane-1-carboxamide |
| 168 | 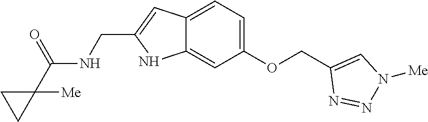 [SMILES: Cn1cc(COc2ccc3cc(CNC(=O)C4(C)CC4)[nH]c3c2)nn1] | 1-methyl-N-((6-((1-methyl-1H-1,2,3- triazol-4-yl)methoxy)-1H-indol-2- yl)methyl)cyclopropane-1-carboxamide |
| 169 | 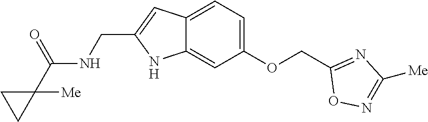 [SMILES: Cc1noc(COc2ccc3cc(CNC(=O)C4(C)CC4)[nH]c3c2)n1] | 1-methyl-N-((6-((3-methyl-1,2,4- oxadiazol-5-yl)methoxy)-1H-indol-2- yl)methyl)cyclopropane-1-carboxamide |
| 170 | 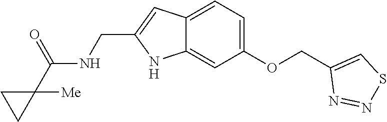 [SMILES: CC1(C(=O)NCc2cc3ccc(OCc4csnn4)cc3[nH]2)CC1] | N-((6-((1,2,3-thiadiazol-4-yl)methoxy)- 1H-indol-2-yl)methyl)-1- methylcyclopropane-1-carboxamide |
| 171 | 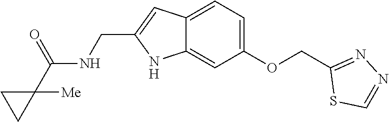 [SMILES: CC1(C(=O)NCc2cc3ccc(OCc4nncs4)cc3[nH]2)CC1] | N-((6-((1,3,4-thiadiazol-2-yl)methoxy)- 1H-indol-2-yl)methyl)-1- methylcyclopropane-1-carboxamide |
| 172 | 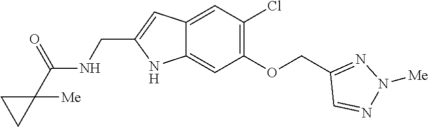 [SMILES: Cn1ncc(COc2cc3[nH]c(CNC(=O)C4(C)CC4)cc3cc2Cl)n1] | N-((5-chloro-6-((2-methyl-2H-1,2,3- triazol-4-yl)methoxy)-1H-indol-2- yl)methyl)-1-methylcyclopropane-1- carboxamide |
| 173 | 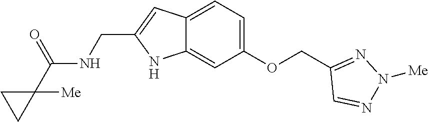 [SMILES: Cn1ncc(COc2ccc3cc(CNC(=O)C4(C)CC4)[nH]c3c2)n1] | 1-methyl-N-((6-((2-methyl-2H-1,2,3- triazol-4-yl)methoxy)-1H-indol-2- yl)methyl)cyclopropane-1-carboxamide |
| 174 | 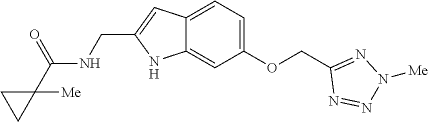 [SMILES: Cn1nnc(COc2ccc3cc(CNC(=O)C4(C)CC4)[nH]c3c2)n1] | 1-methyl-N-((6-((2-methyl-2H-tetrazol- 5-yl)methoxy)-1H-indol-2- yl)methyl)cyclopropane-1-carboxamide |
| 175 | 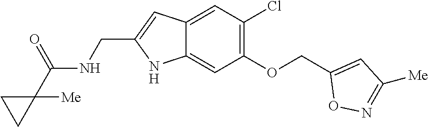 [SMILES: Cc1cc(COc2cc3[nH]c(CNC(=O)C4(C)CC4)cc3cc2Cl)on1] | N-((5-chloro-6-((3-methylisoxazol-5- yl)methoxy)-1H-indol-2-yl)methyl)-1- methylcyclopropane-1-carboxamide |
| 176 | 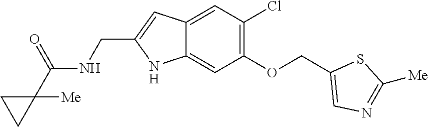 [SMILES: Cc1ncc(COc2cc3[nH]c(CNC(=O)C4(C)CC4)cc3cc2Cl)s1] | N-((5-chloro-6-((2-methylthiazol-5- yl)methoxy)-1H-indol-2-yl)methyl)-1- methylcyclopropane-1-carboxamide |
| 177 | 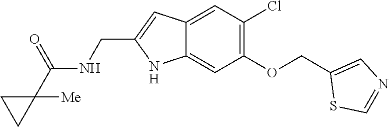 [SMILES: CC1(C(=O)NCc2cc3cc(Cl)c(OCc4cncs4)cc3[nH]2)CC1] | N-((5-chloro-6-(thiazol-5-ylmethoxy)- 1H-indol-2-yl)methyl)-1- methylcyclopropane-1-carboxamide |
| 178 | 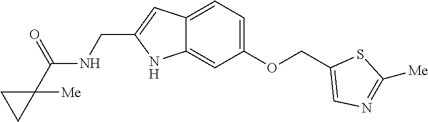 [SMILES: Cc1ncc(COc2ccc3cc(CNC(=O)C4(C)CC4)[nH]c3c2)s1] | 1-methyl-N-((6-((2-methylthiazol-5- yl)methoxy)-1H-indol-2- yl)methyl)cyclopropane-1-carboxamide |
| 179 | 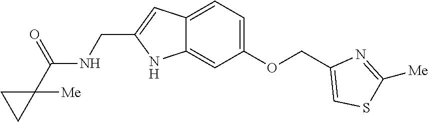 [SMILES: Cc1nc(COc2ccc3cc(CNC(=O)C4(C)CC4)[nH]c3c2)cs1] | 1-methyl-N-((6-((2-methylthiazol-4- yl)methoxy)-1H-indol-2- yl)methyl)cyclopropane-1-carboxamide |
| 180 | 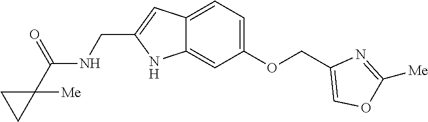 [SMILES: Cc1nc(COc2ccc3cc(CNC(=O)C4(C)CC4)[nH]c3c2)co1] | 1-methyl-N-((6-((2-methyloxazol-4- yl)methoxy)-1H-indol-2- yl)methyl)cyclopropane-1-carboxamide |
| 181 | 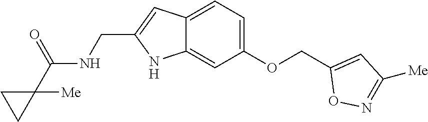 [SMILES: Cc1cc(COc2ccc3cc(CNC(=O)C4(C)CC4)[nH]c3c2)on1] | 1-methyl-N-((6-((3-methylisoxazol-5- yl)methoxy)-1H-indol-2- yl)methyl)cyclopropane-1-carboxamide |
| 182 | 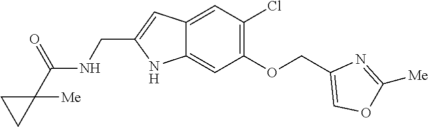 [SMILES: Cc1nc(COc2cc3[nH]c(CNC(=O)C4(C)CC4)cc3cc2Cl)co1] | N-((5-chloro-6-((2-methyloxazol-4- yl)methoxy)-1H-indol-2-yl)methyl)-1- methylcyclopropane-1-carboxamide |
| 183 | 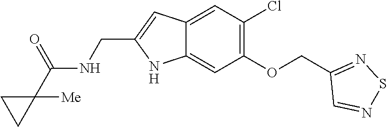 [SMILES: CC1(C(=O)NCc2cc3cc(Cl)c(OCc4cnsn4)cc3[nH]2)CC1] | N-((6-((1,2,5-thiadiazol-3-yl)methoxy)- 5-chloro-1H-indol-2-yl)methyl)-1- methylcyclopropane-1-carboxamide |
| 184 | 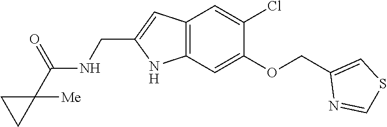 [SMILES: CC1(C(=O)NCc2cc3cc(Cl)c(OCc4cscn4)cc3[nH]2)CC1] | N-((5-chloro-6-(thiazol-4-ylmethoxy)- 1H-indol-2-yl)methyl)-1- methylcyclopropane-1-carboxamide |
| 185 | 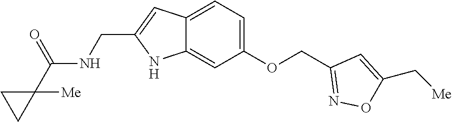 [SMILES: CCc1cc(COc2ccc3cc(CNC(=O)C4(C)CC4)[nH]c3c2)no1] | N-((6-((5-ethylisoxazol-3-yl)methoxy)- 1H-indol-2-yl)methyl)-1- methylcyclopropane-1-carboxamide |
| 186 | 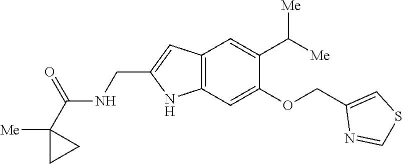 [SMILES: CC(C)c1cc2cc(CNC(=O)C3(C)CC3)[nH]c2cc1OCc1cscn1] | N-((5-isopropyl-6-(thiazol-4- ylmethoxy)-1H-indol-2-yl)methyl)-1- methylcyclopropane-1-carboxamide |
| 187 | 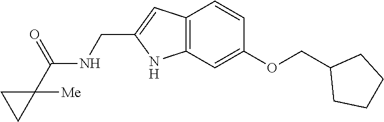 [SMILES: CC1(C(=O)NCc2cc3ccc(OCC4CCCC4)cc3[nH]2)CC1] | N-((6-(cyclopentylmethoxy)-1H-indol- 2-yl)methyl)-1-methylcyclopropane-1- carboxamide |
| 188 | 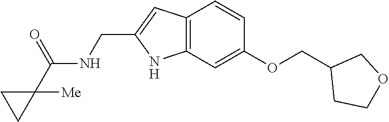 [SMILES: CC1(C(=O)NCc2cc3ccc(OCC4CCOC4)cc3[nH]2)CC1] | 1-methyl-N-((6-((tetrahydrofuran-3- yl)methoxy)-1H-indol-2- yl)methyl)cyclopropane-1-carboxamide |
| 189 | 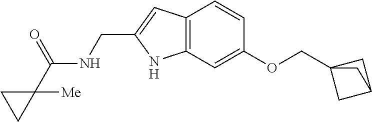 [SMILES: CC1(C(=O)NCc2cc3ccc(OCC45CC(C4)C5)cc3[nH]2)CC1] | N-((6-(bicyclo[1.1.1]pentan-1- ylmethoxy)-1H-indol-2-yl)methyl)-1- methylcyclopropane-1-carboxamide |
| 190 | 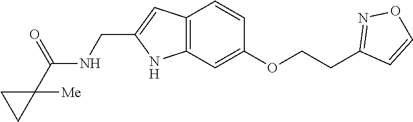 [SMILES: CC1(C(=O)NCc2cc3ccc(OCCc4ccon4)cc3[nH]2)CC1] | N-((6-(2-(isoxazol-3-yl)ethoxy)-1H- indol-2-yl)methyl)-1- methylcyclopropane-1-carboxamide |
| 191 | 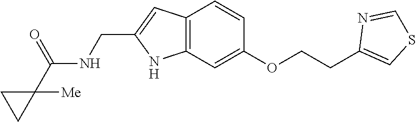 [SMILES: CC1(C(=O)NCc2cc3ccc(OCCc4cscn4)cc3[nH]2)CC1] | 1-methyl-N-((6-(2-(thiazol-4- yl)ethoxy)-1H-indol-2- yl)methyl)cyclopropane-1-carboxamide |
| 192 | 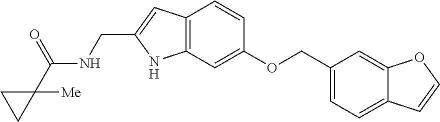 [SMILES: CC1(C(=O)NCc2cc3ccc(OCc4ccc5ccoc5c4)cc3[nH]2)CC1] | N-((6-(benzofuran-6-ylmethoxy)-1H- indol-2-yl)methyl)-1- methylcyclopropane-1-carboxamide |
| 193 | 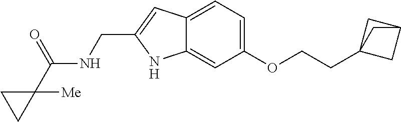 [SMILES: CC1(C(=O)NCc2cc3ccc(OCCC45CC(C4)C5)cc3[nH]2)CC1] | N-((6-(2-(bicyclo[1.1.1]pentan-1- yl)ethoxy)-1H-indol-2-yl)methyl)-1- methylcyclopropane-1-carboxamide |
| 194 | 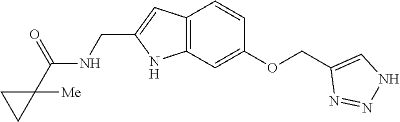 [SMILES: CC1(C(=O)NCc2cc3ccc(OCc4c[nH]nn4)cc3[nH]2)CC1] | N-((6-((1H-1,2,3-triazol-4- yl)methoxy)-1H-indol-2-yl)methyl)-1- methylcyclopropane-1-carboxamide |
| 195 | 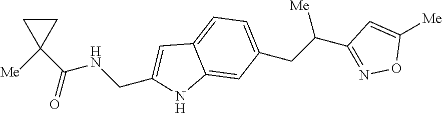 [SMILES: Cc1cc(C(C)Cc2ccc3cc(CNC(=O)C4(C)CC4)[nH]c3c2)no1] | 1-methyl-N-((6-(2-(5-methylisoxazol- 3-yl)propyl)-1H-indol-2- yl)methyl)cyclopropanecarboxamide |
| 196 | 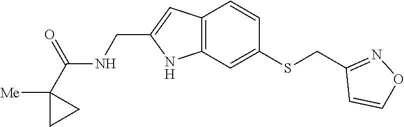 [SMILES: CC1(C(=O)NCc2cc3ccc(SCc4ccon4)cc3[nH]2)CC1] | N-((6-((isoxazol-3-ylmethyl)thio)-1H- indol-2-yl)methyl)-1- methylcyclopropanecarboxamide |
| 197 | 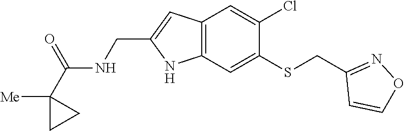 [SMILES: CC1(C(=O)NCc2cc3cc(Cl)c(SCc4ccon4)cc3[nH]2)CC1] | N-((5-chloro-6-((isoxazol-3- ylmethyl)thio)-1H-indol-2-yl)methyl)- 1-methylcyclo propanecarboxamide |
| 198 | 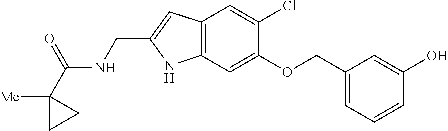 [SMILES: CC1(C(=O)NCc2cc3cc(Cl)c(OCc4cccc(O)c4)cc3[nH]2)CC1] | N-((5-chloro-6-((3-hydroxybenzyl) oxy)-1H-indol-2-yl) methyl)-1- methylcyclopropane-1-carboxamide |
| 199 | 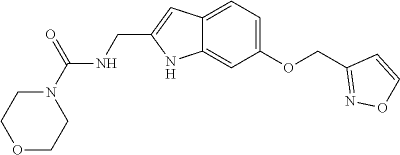 [SMILES: O=C(NCc1cc2ccc(OCc3ccon3)cc2[nH]1)N1CCOCC1] | N-((6-(isoxazol-3-ylmethoxy)-1H- indol-2-yl)methyl)morpholine-4- carboxamide |
| 200 | 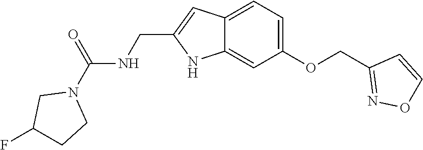 [SMILES: O=C(NCc1cc2ccc(OCc3ccon3)cc2[nH]1)N1CCC(F)C1] | 3-fluoro-N-((6-(isoxazol-3-ylmethoxy)- 1H-indol-2-yl)methyl)pyrrolidine-1- carboxamide |
| 201 | 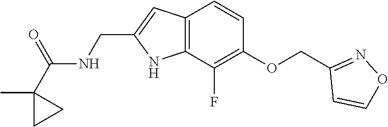 [SMILES: CC1(C(=O)NCc2cc3ccc(OCc4ccon4)c(F)c3[nH]2)CC1] | N-((7-fluoro-6-(isoxazol-3-ylmethoxy)- 1H-indol-2-yl)methyl)-1- methylcyclopropane-1-carboxamide |
| 202 | 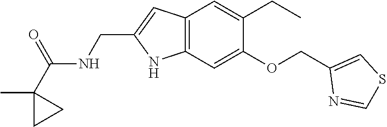 [SMILES: CCc1cc2cc(CNC(=O)C3(C)CC3)[nH]c2cc1OCc1cscn1] | N-((5-ethyl-6-(thiazol-4-ylmethoxy)- 1H-indol-2-yl)methyl)-1- methylcyclopropane-1-carboxamide |
| 203 | 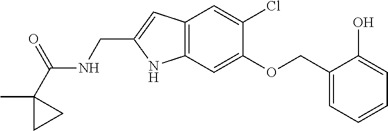 [SMILES: CC1(C(=O)NCc2cc3cc(Cl)c(OCc4ccccc4O)cc3[nH]2)CC1] | N-((5-chloro-6-((2- hydroxybenzyl)oxy)-1H-indol-2- yl)methyl)-1-methylcyclopropane-1- carboxamide |
| 204 | 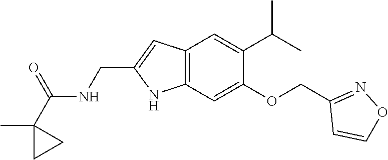 [SMILES: CC(C)c1cc2cc(CNC(=O)C3(C)CC3)[nH]c2cc1OCc1ccon1] | N-((5-isopropyl-6-(isoxazol-3- ylmethoxy)-1H-indol-2-yl)methyl)-1- methylcyclopropane-1-carboxamide |
| 205 | 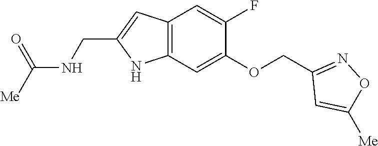 [SMILES: CC(=O)NCc1cc2cc(F)c(OCc3cc(C)on3)cc2[nH]1] | N-((5-fluoro-6-((5-methylisoxazol-3- yl)methoxy)-1H-indol-2- yl)methyl)acetamide |
| 206 | 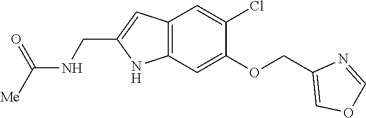 [SMILES: CC(=O)NCc1cc2cc(Cl)c(OCc3cocn3)cc2[nH]1] | N-((5-chloro-6-(oxazol-4-ylmethoxy)- 1H-indol-2-yl)methyl)acetamide |
| 207 | 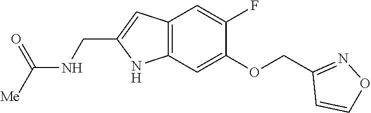 [SMILES: CC(=O)NCc1cc2cc(F)c(OCc3ccon3)cc2[nH]1] | N-((5-fluoro-6-(isoxazol-3-ylmethoxy)- 1H-indol-2-yl)methyl)acetamide |
| 208 | 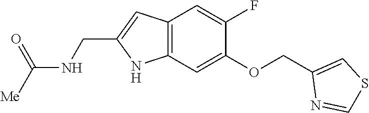 [SMILES: CC(=O)NCc1cc2cc(F)c(OCc3cscn3)cc2[nH]1] | N-((5-fluoro-6-(thiazol-4-ylmethoxy)- 1H-indol-2-yl)methyl)acetamide |
| 209 | 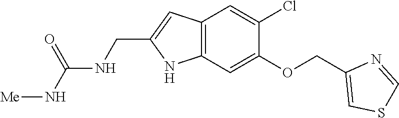 [SMILES: CNC(=O)NCc1cc2cc(Cl)c(OCc3cscn3)cc2[nH]1] | 1-((5-chloro-6-(thiazol-4-ylmethoxy)- 1H-indol-2-yl)methyl)-3-methylurea |
| 210 | 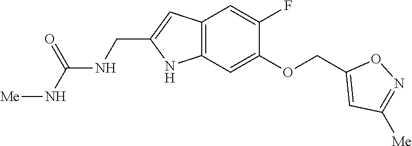 [SMILES: CNC(=O)NCc1cc2cc(F)c(OCc3cc(C)no3)cc2[nH]1] | 1-((5-fluoro-6-((3-methylisoxazol-5- yl)methoxy)-1H-indol-2-yl)methyl)-3- methylurea |
| 211 | 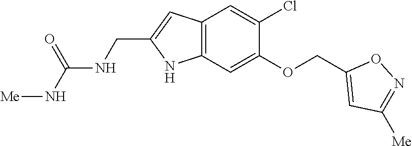 [SMILES: CNC(=O)NCc1cc2cc(Cl)c(OCc3cc(C)no3)cc2[nH]1] | 1-((5-chloro-6-((3-methylisoxazol-5- yl)methoxy)-1H-indol-2-yl)methyl)-3- methylurea |
| 212 | 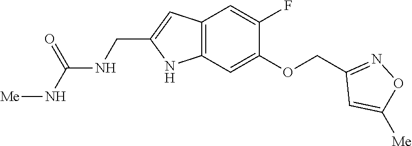 [SMILES: CNC(=O)NCc1cc2cc(F)c(OCc3cc(C)on3)cc2[nH]1] | 1-((5-fluoro-6-((5-methylisoxazol-3- yl)methoxy)-1H-indol-2-yl)methyl)-3- methylurea |
| 213 | 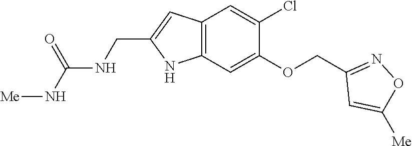 [SMILES: CNC(=O)NCc1cc2cc(Cl)c(OCc3cc(C)on3)cc2[nH]1] | 1-((5-chloro-6-((5-methylisoxazol-3- yl)methoxy)-1H-indol-2-yl)methyl)-3- methylurea |
| 214 | 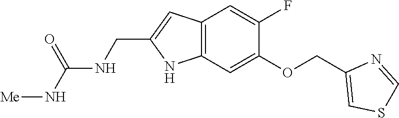 [SMILES: CNC(=O)NCc1cc2cc(F)c(OCc3cscn3)cc2[nH]1] | 1-((5-fluoro-6-(thiazol-4-ylmethoxy)- 1H-indol-2-yl)methyl)-3-methylurea |
| 215 | 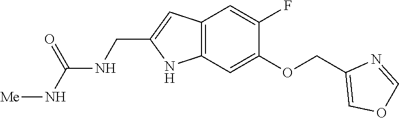 [SMILES: CNC(=O)NCc1cc2cc(F)c(OCc3cocn3)cc2[nH]1] | 1-((5-fluoro-6-(oxazol-4-ylmethoxy)- 1H-indol-2-yl)methyl)-3-methylurea |
| 216 | 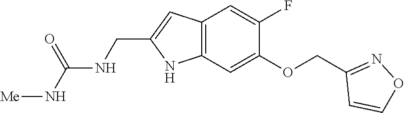 [SMILES: CNC(=O)NCc1cc2cc(F)c(OCc3ccon3)cc2[nH]1] | 1-((5-fluoro-6-(isoxazol-3-ylmethoxy)- 1H-indol-2-yl)methyl)-3-methylurea |
| 217 | 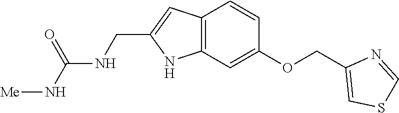 [SMILES: CNC(=O)NCc1cc2ccc(OCc3cscn3)cc2[nH]1] | 1-methyl-3-((6-(thiazol-4-ylmethoxy)- 1H-indol-2-yl)methyl)urea |
| 218 | 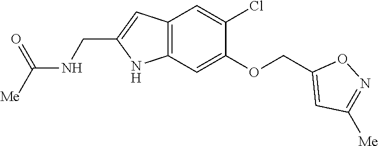 [SMILES: CC(=O)NCc1cc2cc(Cl)c(OCc3cc(C)no3)cc2[nH]1] | N-((5-chloro-6-((3-methylisoxazol-5- yl)methoxy)-1H-indol-2- yl)methyl)acetamide |
| 219 | 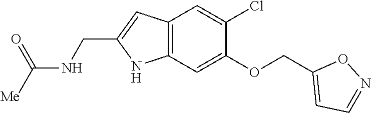 [SMILES: CC(=O)NCc1cc2cc(Cl)c(OCc3ccno3)cc2[nH]1] | N-((5-chloro-6-(isoxazol-5-ylmethoxy)- 1H-indol-2-yl)methyl)acetamide |
| 220 | 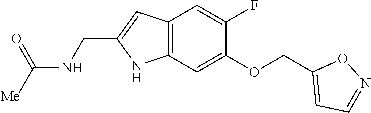 [SMILES: CC(=O)NCc1cc2cc(F)c(OCc3ccno3)cc2[nH]1] | N-((5-fluoro-6-(isoxazol-5-ylmethoxy)- 1H-indol-2-yl)methyl)acetamide |
| 221 | 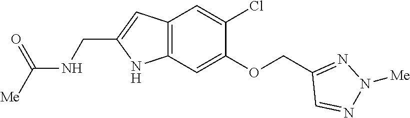 [SMILES: CC(=O)NCc1cc2cc(Cl)c(OCc3cnn(C)n3)cc2[nH]1] | N-((5-chloro-6-((2-methyl-2H-1,2,3- triazol-4-yl)methoxy)-1H-indol-2- yl)methyl)acetamide |
| 222 | 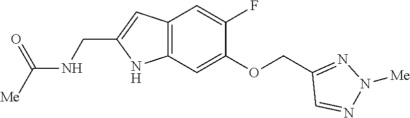 [SMILES: CC(=O)NCc1cc2cc(F)c(OCc3cnn(C)n3)cc2[nH]1] | N-((5-fluoro-6-((2-methyl-2H-1,2,3- triazol-4-yl)methoxy)-1H-indol-2- yl)methyl)acetamide |
| 223 | 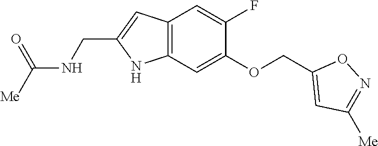 [SMILES: CC(=O)NCc1cc2cc(F)c(OCc3cc(C)no3)cc2[nH]1] | N-((5-fluoro-6-((3-methylisoxazol-5- yl)methoxy)-1H-indol-2- yl)methyl)acetamide |
| 224 | 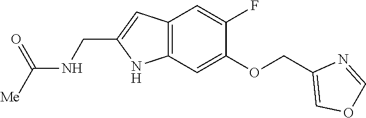 [SMILES: CC(=O)NCc1cc2cc(F)c(OCc3cocn3)cc2[nH]1] | N-((5-fluoro-6-(oxazol-4-ylmethoxy)- 1H-indol-2-yl)methyl)acetamide |
| 225 | 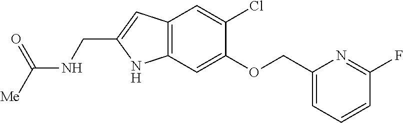 [SMILES: CC(=O)NCc1cc2cc(Cl)c(OCc3cccc(F)n3)cc2[nH]1] | N-((5-chloro-6-((6-fluoropyridin-2- yl)methoxy)-1H-indol-2- yl)methyl)acetamide |
| 226 | 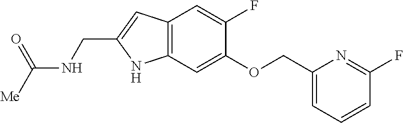 [SMILES: CC(=O)NCc1cc2cc(F)c(OCc3cccc(F)n3)cc2[nH]1] | N-((5-fluoro-6-((6-fluoropyridin-2- yl)methoxy)-1H-indol-2- yl)methyl)acetamide |
| 227 | 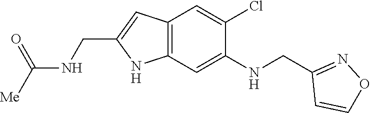 [SMILES: CC(=O)NCc1cc2cc(Cl)c(NCc3ccon3)cc2[nH]1] | N-((5-chloro-6-((isoxazol-3- ylmethyl)amino)-1H-indol-2- yl)methyl)acetamide |
| 228 | 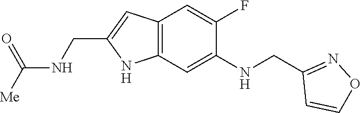 [SMILES: CC(=O)NCc1cc2cc(F)c(NCc3ccon3)cc2[nH]1] | N-((5-fluoro-6-((isoxazol-3- ylmethyl)amino)-1H-indol-2- yl)methyl)acetamide |
| 229 | 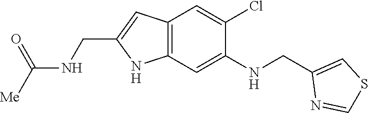 [SMILES: CC(=O)NCc1cc2cc(Cl)c(NCc3cscn3)cc2[nH]1] | N-((5-chloro-6-((thiazol-4- ylmethyl)amino)-1H-indol-2- yl)methyl)acetamide |
| 230 | 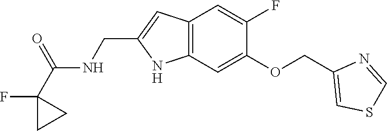 [SMILES: O=C(NCc1cc2cc(F)c(OCc3cscn3)cc2[nH]1)C1(F)CC1] | 1-fluoro-N-((5-fluoro-6-(thiazol-4- ylmethoxy)-1H-indol-2- yl)methyl)cyclopropane-1-carboxamide |
| 231 | 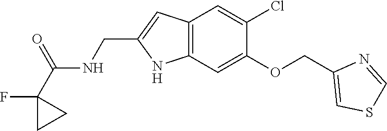 [SMILES: O=C(NCc1cc2cc(Cl)c(OCc3cscn3)cc2[nH]1)C1(F)CC1] | N-((5-chloro-6-(thiazol-4-ylmethoxy)- 1H-indol-2-yl)methyl)-1- fluorocyclopropane-1-carboxamide |
| 232 | 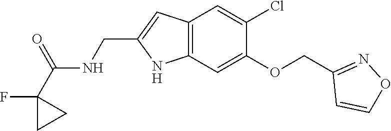 [SMILES: O=C(NCc1cc2cc(Cl)c(OCc3ccon3)cc2[nH]1)C1(F)CC1] | N-((5-chloro-6-(isoxazol-3-ylmethoxy)- 1H-indol-2-yl)methyl)-1- fluorocyclopropane-1-carboxamide |
| 233 | 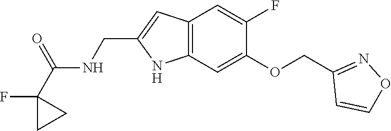 [SMILES: O=C(NCc1cc2cc(F)c(OCc3ccon3)cc2[nH]1)C1(F)CC1] | 1-fluoro-N-((5-fluoro-6-(isoxazol-3- ylmethoxy)-1H-indol-2- yl)methyl)cyclopropane-1-carboxamide |
| 234 | 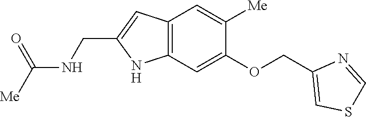 [SMILES: CC(=O)NCc1cc2cc(C)c(OCc3cscn3)cc2[nH]1] | N-((5-methyl-6-(thiazol-4-ylmethoxy)- 1H-indol-2-yl)methyl)acetamide |
| 235 | 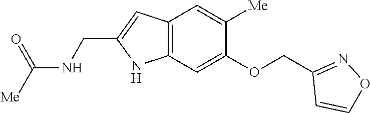 [SMILES: CC(=O)NCc1cc2cc(C)c(OCc3ccon3)cc2[nH]1] | N-((6-(isoxazol-3-ylmethoxy)-5- methyl-1H-indol-2- yl)methyl)acetamide |
| 236 | 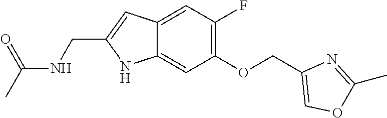 [SMILES: CC(=O)NCc1cc2cc(F)c(OCc3coc(C)n3)cc2[nH]1] | N-((5-fluoro-6-((2-methyloxazol-4- yl)methoxy)-1H-indol-2- yl)methyl)acetamide |
| 237 | 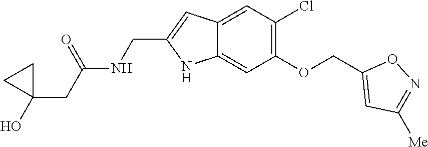 [SMILES: Cc1cc(COc2cc3[nH]c(CNC(=O)CC4(O)CC4)cc3cc2Cl)on1] | N-((5-chloro-6-((3-methylisoxazol-5- yl)methoxy)-1H-indol-2-yl)methyl)-2- (1-hydroxycyclopropyl)acetamide |
| 238 | 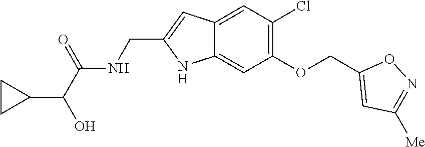 [SMILES: Cc1cc(COc2cc3[nH]c(CNC(=O)C(O)C4CC4)cc3cc2Cl)on1] | N-((5-chloro-6-((3-methylisoxazol-5- yl)methoxy)-1H-indol-2-yl)methyl)-2- cyclopropyl-2-hydroxyacetamide |
| 239 | 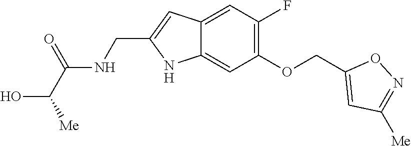 [SMILES: Cc1cc(COc2cc3[nH]c(CNC(=O)[C@H](C)O)cc3cc2F)on1] | (S)-N-((5-fluoro-6-((3-methylisoxazol- 5-yl)methoxy)-1H-indol-2-yl)methyl)- 2-hydroxypropanamide |
| 240 | 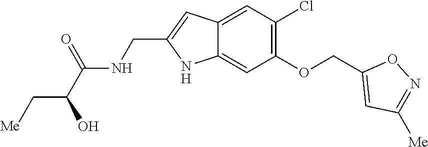 [SMILES: CC[C@H](O)C(=O)NCc1cc2cc(Cl)c(OCc3cc(C)no3)cc2[nH]1] | (S)-N-((5-chloro-6-((3-methylisoxazol- 5-yl)methoxy)-1H-indol-2-yl)methyl)- 2-hydroxybutanamide |
| 241 | 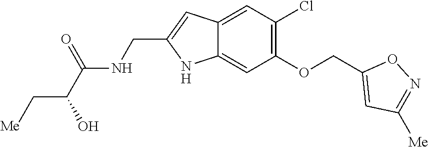 [SMILES: CC[C@@H](O)C(=O)NCc1cc2cc(Cl)c(OCc3cc(C)no3)cc2[nH]1] | (R)-N-((5-chloro-6-((3-methylisoxazol- 5-yl)methoxy)-1H-indol-2-yl)methyl)- 2-hydroxybutanamide |
| 242 | 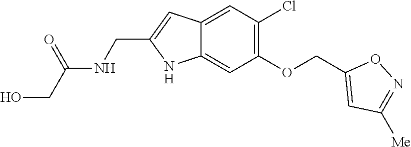 [SMILES: Cc1cc(COc2cc3[nH]c(CNC(=O)CO)cc3cc2Cl)on1] | N-((5-chloro-6-((3-methylisoxazol-5- yl)methoxy)-1H-indol-2-yl)methyl)-2- hydroxyacetamide |
| 243 | 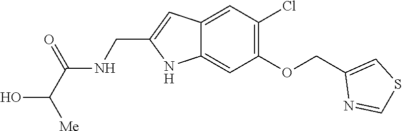 [SMILES: CC(O)C(=O)NCc1cc2cc(Cl)c(OCc3cscn3)cc2[nH]1] | N-((5-chloro-6-(thiazol-4-ylmethoxy)- 1H-indol-2-yl)methyl)-2- hydroxypropanamide |
| 244 | 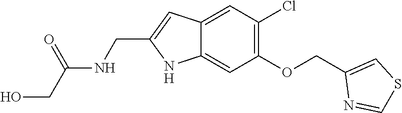 [SMILES: O=C(CO)NCc1cc2cc(Cl)c(OCc3cscn3)cc2[nH]1] | N-((5-chloro-6-(thiazol-4-ylmethoxy)- 1H-indol-2-yl)methyl)-2- hydroxyacetamide |
| 245 | 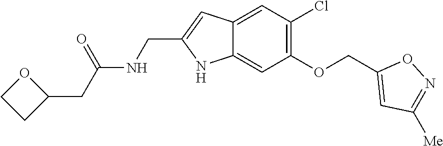 [SMILES: Cc1cc(COc2cc3[nH]c(CNC(=O)CC4CCO4)cc3cc2Cl)on1] | N-((5-chloro-6-((3-methylisoxazol-5- yl)methoxy)-1H-indol-2-yl)methyl)-2- (oxetan-2-yl)acetamide |
| 246 | 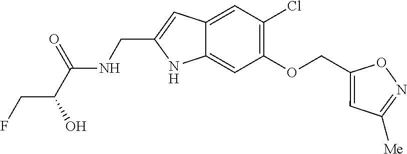 [SMILES: Cc1cc(COc2cc3[nH]c(CNC(=O)[C@H](O)CF)cc3cc2Cl)on1] | (S)-N-((5-chloro-6-((3-methylisoxazol- 5-yl)methoxy)-1H-indol-2-yl)methyl)- 3-fluoro-2-hydroxypropanamide |
| 247 | 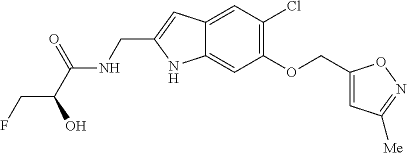 [SMILES: Cc1cc(COc2cc3[nH]c(CNC(=O)[C@@H](O)CF)cc3cc2Cl)on1] | (R)-N-((5-chloro-6-((3-methylisoxazol- 5-yl)methoxy)-1H-indol-2-yl)methyl)- 3-fluoro-2-hydroxypropanamide |
| 248 | 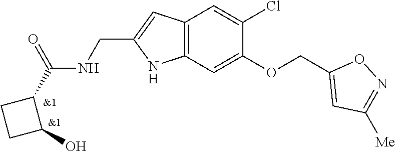 [SMILES: Cc1cc(COc2cc3[nH]c(CNC(=O)[C@H]4CC[C@@H]4O)cc3cc2Cl)on1] | (1R,2R)-N-((5-chloro-6-((3- methylisoxazol-5-yl)methoxy)-1H- indol-2-yl)methyl)-2- hydroxycyclobutane-1-carboxamide and (1S,2S)-N-((5-chloro-6-((3- methylisoxazol-5-yl)methoxy)-1H- indol-2-yl)methyl)-2- hydroxycyclobutane-1-carboxamide |
| 249 | 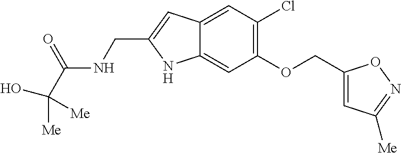 [SMILES: Cc1cc(COc2cc3[nH]c(CNC(=O)C(C)(C)O)cc3cc2Cl)on1] | N-((5-chloro-6-((3-methylisoxazol-5- yl)methoxy)-1H-indol-2-yl)methyl)-2- hydroxy-2-methylpropanamide |
| 250 | 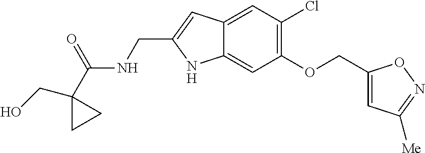 [SMILES: Cc1cc(COc2cc3[nH]c(CNC(=O)C4(CO)CC4)cc3cc2Cl)on1] | N-((5-chloro-6-((3-methylisoxazol-5- yl)methoxy)-1H-indol-2-yl)methyl)-1- (hydroxymethyl)cyclopropane-1- carboxamide |
| 251 | 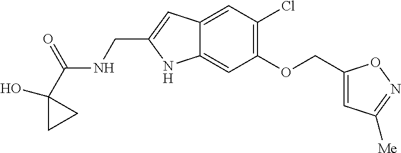 [SMILES: Cc1cc(COc2cc3[nH]c(CNC(=O)C4(O)CC4)cc3cc2Cl)on1] | N-((5-chloro-6-((3-methylisoxazol-5- yl)methoxy)-1H-indol-2-yl)methyl)-1- hydroxycyclopropane-1-carboxamide |
| 252 | 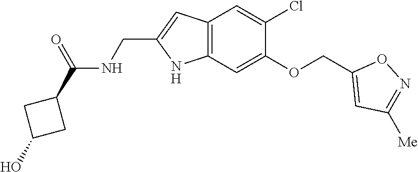 [SMILES: Cc1cc(COc2cc3[nH]c(CNC(=O)[C@H]4C[C@H](O)C4)cc3cc2Cl)on1] | (1R,3R)-N-((5-chloro-6-((3- methylisoxazol-5-yl)methoxy)-1H- indol-2-yl)methyl)-3- hydroxycyclobutane-1-carboxamide |
| 253 | 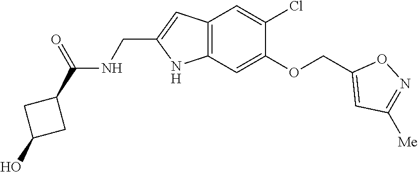 [SMILES: Cc1cc(COc2cc3[nH]c(CNC(=O)[C@H]4C[C@@H](O)C4)cc3cc2Cl)on1] | (1S,3S)-N-((5-chloro-6-((3- methylisoxazol-5-yl)methoxy)-1H- indol-2-yl)methyl)-3- hydroxycyclobutane-1-carboxamide |
| 254 | 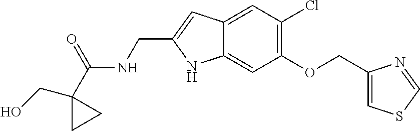 [SMILES: O=C(NCc1cc2cc(Cl)c(OCc3cscn3)cc2[nH]1)C1(CO)CC1] | N-((5-chloro-6-(thiazol-4-ylmethoxy)- 1H-indol-2-yl)methyl)-1- (hydroxymethyl)cyclopropane-1- carboxamide |
| 255 | 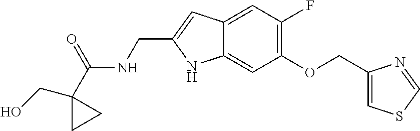 [SMILES: O=C(NCc1cc2cc(F)c(OCc3cscn3)cc2[nH]1)C1(CO)CC1] | N-((5-fluoro-6-(thiazol-4-ylmethoxy)- 1H-indol-2-yl)methyl)-1- (hydroxymethyl)cyclopropane-1- carboxamide |
| 256 | 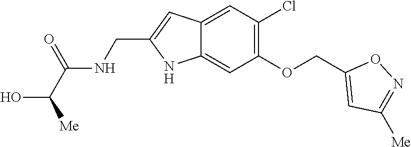 [SMILES: Cc1cc(COc2cc3[nH]c(CNC(=O)[C@@H](C)O)cc3cc2Cl)on1] | (R)-N-((5-chloro-6-((3-methylisoxazol- 5-yl)methoxy)-1H-indol-2-yl)methyl)- 2-hydroxypropanamide |
| 257 | 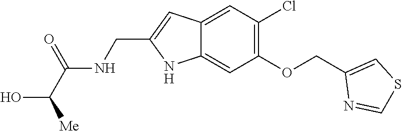 [SMILES: C[C@@H](O)C(=O)NCc1cc2cc(Cl)c(OCc3cscn3)cc2[nH]1] | (R)-N-((5-chloro-6-(thiazol-4- ylmethoxy)-1H-indol-2-yl)methyl)-2- hydroxypropanamide |
| 258 | 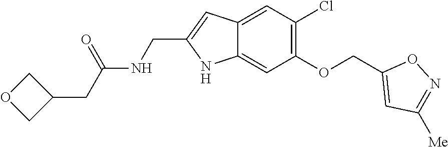 [SMILES: Cc1cc(COc2cc3[nH]c(CNC(=O)CC4COC4)cc3cc2Cl)on1] | N-((5-chloro-6-((3-methylisoxazol-5- yl)methoxy)-1H-indol-2-yl)methyl)-2- (oxetan-3-yl)acetamide |
| 259 | 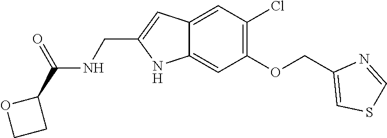 [SMILES: O=C(NCc1cc2cc(Cl)c(OCc3cscn3)cc2[nH]1)[C@H]1CCO1] | (R)-N-((5-chloro-6-(thiazol-4- ylmethoxy)-1H-indol-2- yl)methyl)oxetane-2-carboxamide |
| 260 | 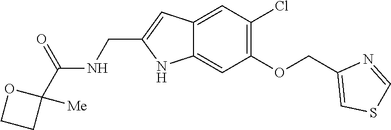 [SMILES: CC1(C(=O)NCc2cc3cc(Cl)c(OCc4cscn4)cc3[nH]2)CCO1] | N-((5-chloro-6-(thiazol-4-ylmethoxy)- 1H-indol-2-yl)methyl)-2- methyloxetane-2-carboxamide |
| 261 | 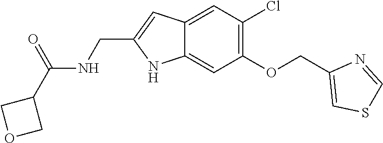 [SMILES: O=C(NCc1cc2cc(Cl)c(OCc3cscn3)cc2[nH]1)C1COC1] | N-((5-chloro-6-(thiazol-4-ylmethoxy)- 1H-indol-2-yl)methyl)oxetane-3- carboxamide |
| 262 | 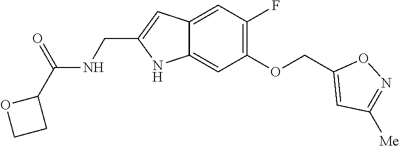 [SMILES: Cc1cc(COc2cc3[nH]c(CNC(=O)C4CCO4)cc3cc2F)on1] | N-((5-fluoro-6-((3-methylisoxazol-5- yl)methoxy)-1H-indol-2- yl)methyl)oxetane-2-carboxamide |
| 263 | 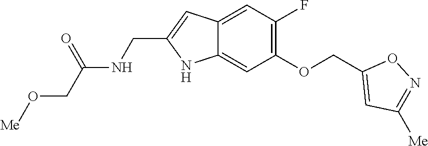 [SMILES: COCC(=O)NCc1cc2cc(F)c(OCc3cc(C)no3)cc2[nH]1] | N-((5-fluoro-6-((3-methylisoxazol-5- yl)methoxy)-1H-indol-2-yl)methyl)-2- methoxyacetamide |
| 264 | 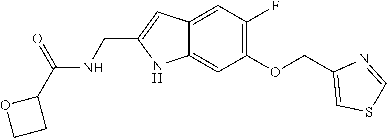 [SMILES: O=C(NCc1cc2cc(F)c(OCc3cscn3)cc2[nH]1)C1CCO1] | N-((5-fluoro-6-(thiazol-4-ylmethoxy)- 1H-indol-2-yl)methyl)oxetane-2- carboxamide |
| 265 | 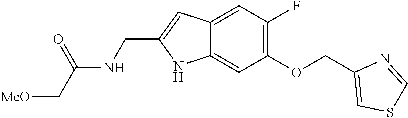 [SMILES: COCC(=O)NCc1cc2cc(F)c(OCc3cscn3)cc2[nH]1] | N-((5-fluoro-6-(thiazol-4-ylmethoxy)- 1H-indol-2-yl)methyl)-2- methoxyacetamide |
| 266 | 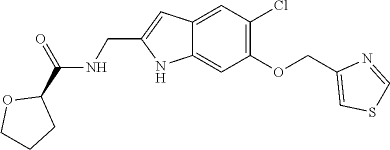 [SMILES: O=C(NCc1cc2cc(Cl)c(OCc3cscn3)cc2[nH]1)[C@H]1CCCO1] | (R)-N-((5-chloro-6-(thiazol-4- ylmethoxy)-1H-indol-2- yl)methyl)tetrahydrofuran-2- carboxamide |
| 267 | 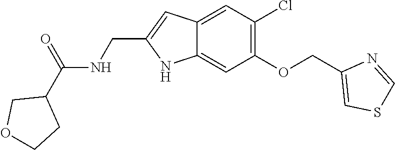 [SMILES: O=C(NCc1cc2cc(Cl)c(OCc3cscn3)cc2[nH]1)C1CCOC1] | N-((5-chloro-6-(thiazol-4-ylmethoxy)- 1H-indol-2-yl)methyl)tetrahydrofuran- 3-carboxamide |
| 268 | 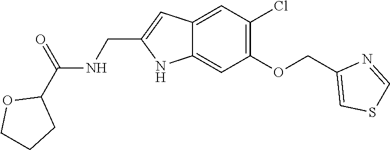 [SMILES: O=C(NCc1cc2cc(Cl)c(OCc3cscn3)cc2[nH]1)C1CCCO1] | N-((5-chloro-6-(thiazol-4-ylmethoxy)- 1H-indol-2-yl)methyl)tetrahydrofuran- 2-carboxamide |
| 269 | 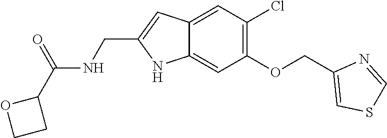 [SMILES: O=C(NCc1cc2cc(Cl)c(OCc3cscn3)cc2[nH]1)C1CCO1] | N-((5-chloro-6-(thiazol-4-ylmethoxy)- 1H-indol-2-yl)methyl)oxetane-2- carboxamide |
| 270 | 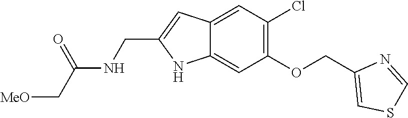 [SMILES: COCC(=O)NCc1cc2cc(Cl)c(OCc3cscn3)cc2[nH]1] | N-((5-chloro-6-(thiazol-4-ylmethoxy)- 1H-indol-2-yl)methyl)-2- methoxyacetamide |
| 271 | 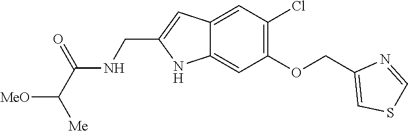 [SMILES: COC(C)C(=O)NCc1cc2cc(Cl)c(OCc3cscn3)cc2[nH]1] | N-((5-chloro-6-(thiazol-4-ylmethoxy)- 1H-indol-2-yl)methyl)-2- methoxypropanamide |
| 272 | 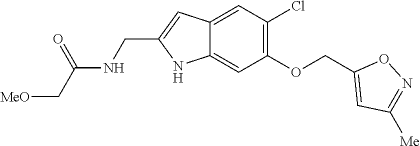 [SMILES: COCC(=O)NCc1cc2cc(Cl)c(OCc3cc(C)no3)cc2[nH]1] | N-((5-chloro-6-((3-methylisoxazol-5- yl)methoxy)-1H-indol-2-yl)methyl)-2- methoxyacetamide |
| 273 | 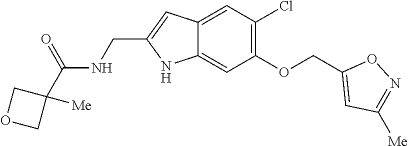 [SMILES: Cc1cc(COc2cc3[nH]c(CNC(=O)C4(C)COC4)cc3cc2Cl)on1] | N-((5-chloro-6-((3-methylisoxazol-5- yl)methoxy)-1H-indol-2-yl)methyl)-3- methyloxetane-3-carboxamide |
| 274 | 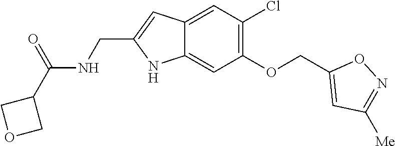 [SMILES: Cc1cc(COc2cc3[nH]c(CNC(=O)C4COC4)cc3cc2Cl)on1] | N-((5-chloro-6-((3-methylisoxazol-5- yl)methoxy)-1H-indol-2- yl)methyl)oxetane-3-carboxamide |
| 275 | 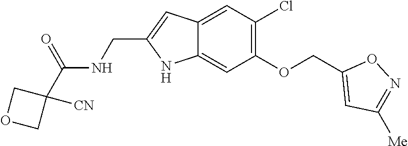 [SMILES: Cc1cc(COc2cc3[nH]c(CNC(=O)C4(C#N)COC4)cc3cc2Cl)on1] | N-((5-chloro-6-((3-methylisoxazol-5- yl)methoxy)-1H-indol-2-yl)methyl)-3- cyanooxetane-3-carboxamide |
| 276 | 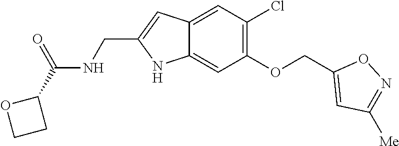 [SMILES: Cc1cc(COc2cc3[nH]c(CNC(=O)[C@@H]4CCO4)cc3cc2Cl)on1] | (S)-N-((5-chloro-6-((3-methylisoxazol- 5-yl)methoxy)-1H-indol-2- yl)methyl)oxetane-2-carboxamide |
| 277 | 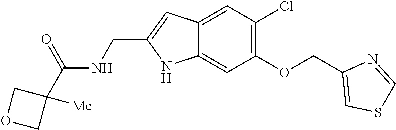 [SMILES: CC1(C(=O)NCc2cc3cc(Cl)c(OCc4cscn4)cc3[nH]2)COC1] | N-((5-chloro-6-(thiazol-4-ylmethoxy)- 1H-indol-2-yl)methyl)-3- methyloxetane-3-carboxamide |
| 278 | 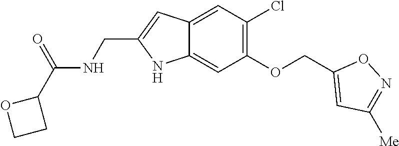 [SMILES: Cc1cc(COc2cc3[nH]c(CNC(=O)C4CCO4)cc3cc2Cl)on1] | N-((5-chloro-6-((3-methylisoxazol-5- yl)methoxy)-1H-indol-2- yl)methyl)oxetane-2-carboxamide |
| 279 | 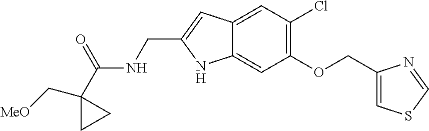 [SMILES: COCC1(C(=O)NCc2cc3cc(Cl)c(OCc4cscn4)cc3[nH]2)CC1] | N-((5-chloro-6-(thiazol-4-ylmethoxy)- 1H-indol-2-yl)methyl)-1- (methoxymethyl)cyclopropane-1- carboxamide |
| 280 | 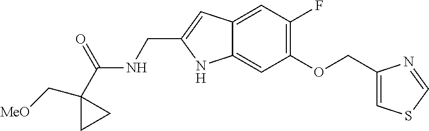 [SMILES: COCC1(C(=O)NCc2cc3cc(F)c(OCc4cscn4)cc3[nH]2)CC1] | N-((5-fluoro-6-(thiazol-4-ylmethoxy)- 1H-indol-2-yl)methyl)-1- (methoxymethyl)cyclopropane-1- carboxamide |
| 281 | 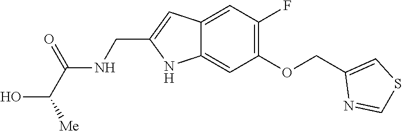 [SMILES: C[C@H](O)C(=O)NCc1cc2cc(F)c(OCc3cscn3)cc2[nH]1] | (S)-N-((5-fluoro-6-(thiazol-4- ylmethoxy)-1H-indol-2-yl)methyl)-2- hydroxypropanamide |
| 282 | 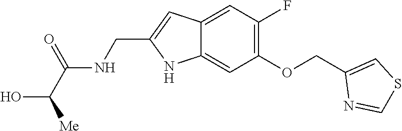 [SMILES: C[C@@H](O)C(=O)NCc1cc2cc(F)c(OCc3cscn3)cc2[nH]1] | (R)-N-((5-fluoro-6-(thiazol-4- ylmethoxy)-1H-indol-2-yl)methyl)-2- hydroxypropanamide |
| 283 | 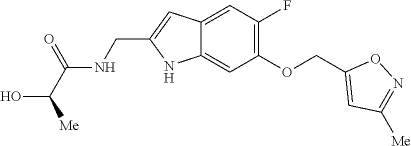 [SMILES: Cc1cc(COc2cc3[nH]c(CNC(=O)[C@@H](C)O)cc3cc2F)on1] | (R)-N-((5-fluoro-6-((3-methylisoxazol- 5-yl)methoxy)-1H-indol-2-yl)methyl)- 2-hydroxypropanamide |
| 284 | 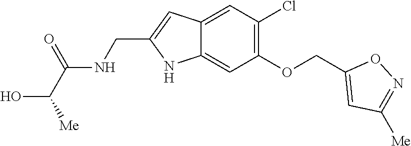 [SMILES: Cc1cc(COc2cc3[nH]c(CNC(=O)[C@H](C)O)cc3cc2Cl)on1] | (S)-N-((5-chloro-6-((3-methylisoxazol- 5-yl)methoxy)-1H-indol-2-yl)methyl)- 2-hydroxypropanamide |
| 285 | 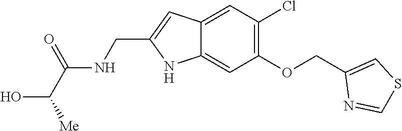 [SMILES: C[C@H](O)C(=O)NCc1cc2cc(Cl)c(OCc3cscn3)cc2[nH]1] | (S)-N-((5-chloro-6-(thiazol-4- ylmethoxy)-1H-indol-2-yl)methyl)-2- hydroxypropanamide |
| 286 | 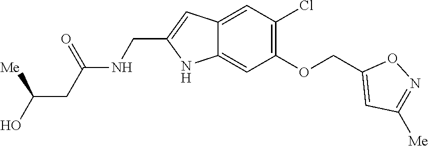 [SMILES: Cc1cc(COc2cc3[nH]c(CNC(=O)C[C@H](C)O)cc3cc2Cl)on1] | (S)-N-((5-chloro-6-((3-methylisoxazol- 5-yl)methoxy)-1H-indol-2-yl)methyl)- 3-hydroxybutanamide |
| 287 | 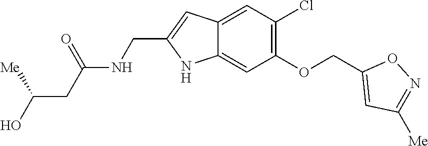 [SMILES: Cc1cc(COc2cc3[nH]c(CNC(=O)C[C@@H](C)O)cc3cc2Cl)on1] | (R)-N-((5-chloro-6-((3-methylisoxazol- 5-yl)methoxy)-1H-indol-2-yl)methyl)- 3-hydroxybutanamide |
| 288 | 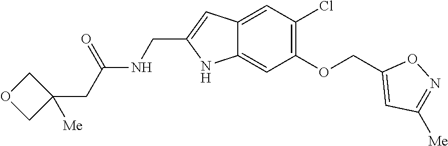 [SMILES: Cc1cc(COc2cc3[nH]c(CNC(=O)CC4(C)COC4)cc3cc2Cl)on1] | N-((5-chloro-6-((3-methylisoxazol-5- yl)methoxy)-1H-indol-2-yl)methyl)-2- (3-methyloxetan-3-yl)acetamide |
| 289 | 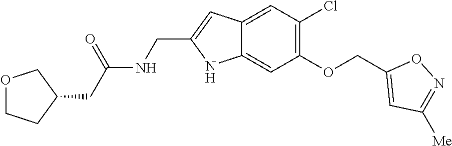 [SMILES: Cc1cc(COc2cc3[nH]c(CNC(=O)C[C@@H]4CCOC4)cc3cc2Cl)on1] | (S)-N-((5-chloro-6-((3-methylisoxazol- 5-yl)methoxy)-1H-indol-2-yl)methyl)- 2-(tetrahydrofuran-3-yl)acetamide |
| 290 | 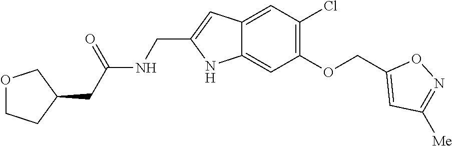 [SMILES: Cc1cc(COc2cc3[nH]c(CNC(=O)C[C@H]4CCOC4)cc3cc2Cl)on1] | (R)-N-((5-chloro-6-((3-methylisoxazol- 5-yl)methoxy)-1H-indol-2-yl)methyl)- 2-(tetrahydrofuran-3-yl)acetamide |
| 291 | 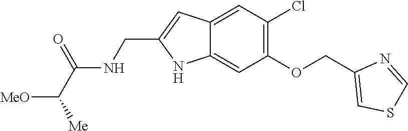 [SMILES: CO[C@@H](C)C(=O)NCc1cc2cc(Cl)c(OCc3cscn3)cc2[nH]1] | (S)-N-((5-chloro-6-(thiazol-4- ylmethoxy)-1H-indol-2-yl)methyl)-2- methoxypropanamide |
| 292 | 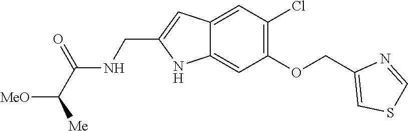 [SMILES: CO[C@H](C)C(=O)NCc1cc2cc(Cl)c(OCc3cscn3)cc2[nH]1] | (R)-N-((5-chloro-6-(thiazol-4- ylmethoxy)-1H-indol-2-yl)methyl)-2- methoxypropanamide |
| 293 | 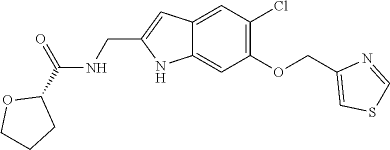 [SMILES: O=C(NCc1cc2cc(Cl)c(OCc3cscn3)cc2[nH]1)[C@@H]1CCCO1] | (S)-N-((5-chloro-6-(thiazol-4- ylmethoxy)-1H-indol-2- yl)methyl)tetrahydrofuran-2- carboxamide |
| 294 | 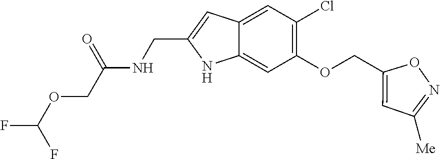 [SMILES: Cc1cc(COc2cc3[nH]c(CNC(=O)COC(F)F)cc3cc2Cl)on1] | N-((5-chloro-6-((3-methylisoxazol-5- yl)methoxy)-1H-indol-2-yl)methyl)-2- (difluoromethoxy)acetamide |
| 295 | 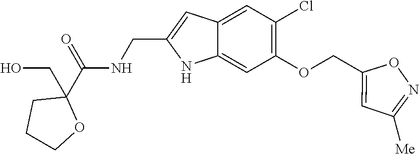 [SMILES: Cc1cc(COc2cc3[nH]c(CNC(=O)C4(CO)CCCO4)cc3cc2Cl)on1] | N-((5-chloro-6-((3-methylisoxazol-5- yl)methoxy)-1H-indol-2-yl)methyl)-2- (hydroxymethyl)tetrahydrofuran-2- carboxamide |
| 296 | 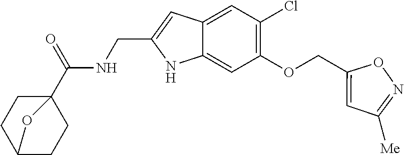 [SMILES: Cc1cc(COc2cc3[nH]c(CNC(=O)C45CCC(CC4)O5)cc3cc2Cl)on1] | N-((5-chloro-6-((3-methylisoxazol-5- yl)methoxy)-1H-indol-2-yl)methyl)-7- oxabicyclo[2.2.1]heptane-1- carboxamide |
| 297 | 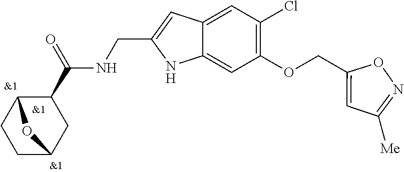 [SMILES: Cc1cc(COc2cc3[nH]c(CNC(=O)[C@H]4C[C@@H]5CC[C@H]4O5)cc3cc2Cl)on1] | (1R,2S,4S)-N-((5-chloro-6-((3- methylisoxazol-5-yl)methoxy)-1H- indol-2-yl)methyl)-7- oxabicyclo[2.2.1]heptane-2- carboxamide and (1S,2R,4R)-N-((5- chloro-6-((3-methylisoxazol-5- yl)methoxy)-1H-indol-2-yl)methyl)-7- oxabicyclo[2.2.1]heptane-2- carboxamide |
| 298 | 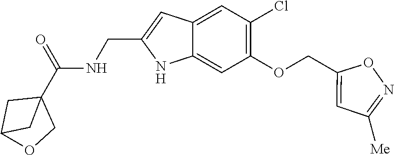 [SMILES: Cc1cc(COc2cc3[nH]c(CNC(=O)C45COC(C4)C5)cc3cc2Cl)on1] | N-((5-chloro-6-((3-methylisoxazol-5- yl)methoxy)-1H-indol-2-yl)methyl)-2- oxabicyclo[2.1.1]hexane-4- carboxamide |
| 299 | 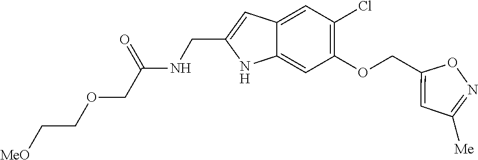 [SMILES: COCCOCC(=O)NCc1cc2cc(Cl)c(OCc3cc(C)no3)cc2[nH]1] | N-((5-chloro-6-((3-methylisoxazol-5- yl)methoxy)-1H-indol-2-yl)methyl)-2- (2-methoxyethoxy)acetamide |
| 300 | 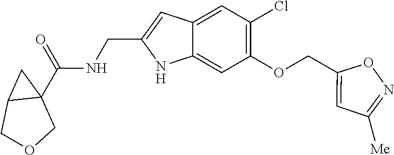 [SMILES: Cc1cc(COc2cc3[nH]c(CNC(=O)C45COCC4C5)cc3cc2Cl)on1] | N-((5-chloro-6-((3-methylisoxazol-5- yl)methoxy)-1H-indol-2-yl)methyl)-3- oxabicyclo[3.1.0]hexane-1- carboxamide |
| 301 | 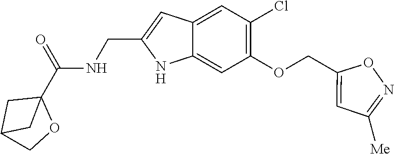 [SMILES: Cc1cc(COc2cc3[nH]c(CNC(=O)C45CC(CO4)C5)cc3cc2Cl)on1] | N-((5-chloro-6-((3-methylisoxazol-5- yl)methoxy)-1H-indol-2-yl)methyl)-2- oxabicyclo[2.1.1]hexane-1- carboxamide |
| 302 | 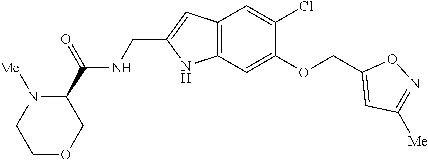 [SMILES: Cc1cc(COc2cc3[nH]c(CNC(=O)[C@H]4COCCN4C)cc3cc2Cl)on1] | (R)-N-((5-chloro-6-((3-methylisoxazol- 5-yl)methoxy)-1H-indol-2-yl)methyl)- 4-methylmorpholine-3-carboxamide |
| 303 | 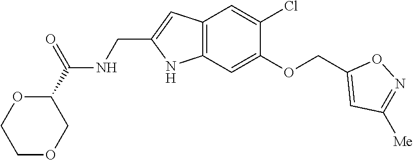 [SMILES: Cc1cc(COc2cc3[nH]c(CNC(=O)[C@@H]4COCCO4)cc3cc2Cl)on1] | (S)-N-((5-chloro-6-((3-methylisoxazol- 5-yl)methoxy)-1H-indol-2-yl)methyl)- 1,4-dioxane-2-carboxamide |
| 304 | 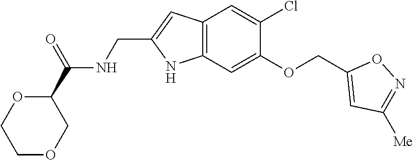 [SMILES: Cc1cc(COc2cc3[nH]c(CNC(=O)[C@H]4COCCO4)cc3cc2Cl)on1] | (R)-N-((5-chloro-6-((3-methylisoxazol- 5-yl)methoxy)-1H-indol-2-yl)methyl)- 1,4-dioxane-2-carboxamide |
| 305 | 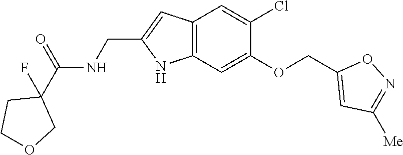 [SMILES: Cc1cc(COc2cc3[nH]c(CNC(=O)C4(F)CCOC4)cc3cc2Cl)on1] | N-((5-chloro-6-((3-methylisoxazol-5- yl)methoxy)-1H-indol-2-yl)methyl)-3- fluorotetrahydrofuran-3-carboxamide |
| 306 | 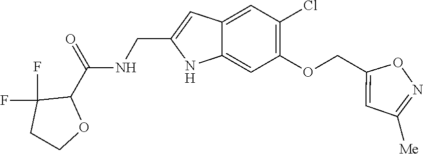 [SMILES: Cc1cc(COc2cc3[nH]c(CNC(=O)C4OCCC4(F)F)cc3cc2Cl)on1] | N-((5-chloro-6-((3-methylisoxazol-5- yl)methoxy)-1H-indol-2-yl)methyl)- 3,3-difluorotetrahydrofuran-2- carboxamide |
| 307 | 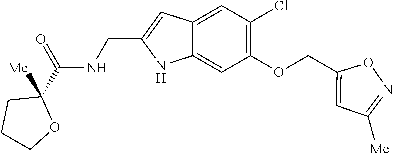 [SMILES: Cc1cc(COc2cc3[nH]c(CNC(=O)[C@@]4(C)CCCO4)cc3cc2Cl)on1] | (R)-N-((5-chloro-6-((3-methylisoxazol- 5-yl)methoxy)-1H-indol-2-yl)methyl)- 2-methyltetrahydrofuran-2- carboxamide |
| 308 | 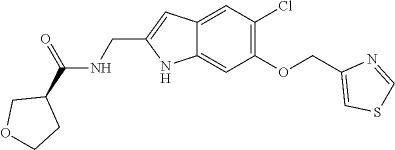 [SMILES: O=C(NCc1cc2cc(Cl)c(OCc3cscn3)cc2[nH]1)[C@H]1CCOC1] | (S)-N-((5-chloro-6-(thiazol-4- ylmethoxy)-1H-indol-2- yl)methyl)tetrahydrofuran-3- carboxamide |
| 309 | 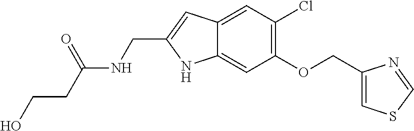 [SMILES: O=C(CCO)NCc1cc2cc(Cl)c(OCc3cscn3)cc2[nH]1] | N-((5-chloro-6-(thiazol-4-ylmethoxy)- 1H-indol-2-yl)methyl)-3- hydroxypropanamide |
| 310 | 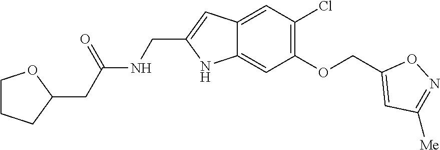 [SMILES: Cc1cc(COc2cc3[nH]c(CNC(=O)CC4CCCO4)cc3cc2Cl)on1] | N-((5-chloro-6-((3-methylisoxazol-5- yl)methoxy)-1H-indol-2-yl)methyl)-2- (tetrahydrofuran-2-yl)acetamide |
| 311 | 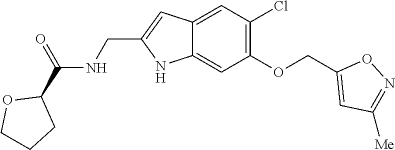 [SMILES: Cc1cc(COc2cc3[nH]c(CNC(=O)[C@H]4CCCO4)cc3cc2Cl)on1] | (R)-N-((5-chloro-6-((3-methylisoxazol- 5-yl)methoxy)-1H-indol-2- yl)methyl)tetrahydrofuran-2- carboxamide |
| 312 | 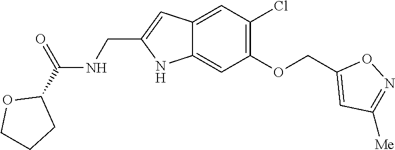 [SMILES: Cc1cc(COc2cc3[nH]c(CNC(=O)[C@@H]4CCCO4)cc3cc2Cl)on1] | (S)-N-((5-chloro-6-((3-methylisoxazol- 5-yl)methoxy)-1H-indol-2- yl)methyl)tetrahydrofuran-2- carboxamide |
| 313 | 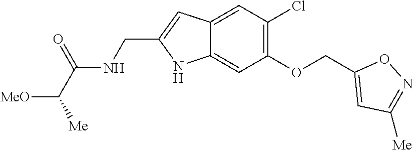 [SMILES: CO[C@@H](C)C(=O)NCc1cc2cc(Cl)c(OCc3cc(C)no3)cc2[nH]1] | (S)-N-((5-chloro-6-((3-methylisoxazol- 5-yl)methoxy)-1H-indol-2-yl)methyl)- 2-methoxypropanamide |
| 314 | 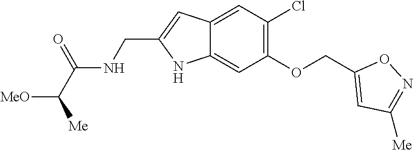 [SMILES: CO[C@H](C)C(=O)NCc1cc2cc(Cl)c(OCc3cc(C)no3)cc2[nH]1] | (R)-N-((5-chloro-6-((3-methylisoxazol- 5-yl)methoxy)-1H-indol-2-yl)methyl)- 2-methoxypropanamide |
| 315 | 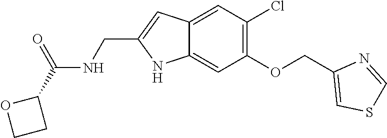 [SMILES: O=C(NCc1cc2cc(Cl)c(OCc3cscn3)cc2[nH]1)[C@@H]1CCO1] | (S)-N-((5-chloro-6-(thiazol-4- ylmethoxy)-1H-indol-2- yl)methyl)oxetane-2-carboxamide |
| 316 | 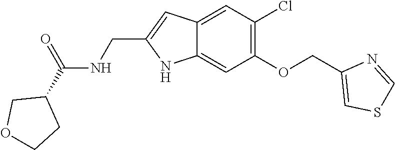 [SMILES: O=C(NCc1cc2cc(Cl)c(OCc3cscn3)cc2[nH]1)[C@@H]1CCOC1] | (R)-N-((5-chloro-6-(thiazol-4- ylmethoxy)-1H-indol-2- yl)methyl)tetrahydrofuran-3- carboxamide |
| 317 | 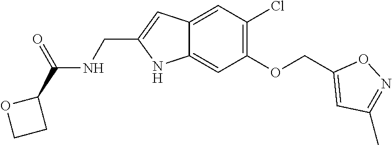 [SMILES: Cc1cc(COc2cc3[nH]c(CNC(=O)[C@H]4CCO4)cc3cc2Cl)on1] | (R)-N-((5-chloro-6-((3-methylisoxazol- 5-yl)methoxy)-1H-indol-2- yl)methyl)oxetane-2-carboxamide |
| 318 | 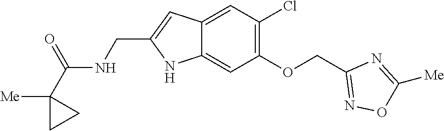 [SMILES: Cc1nc(COc2cc3[nH]c(CNC(=O)C4(C)CC4)cc3cc2Cl)no1] | N-((5-chloro-6-((5-methyl-1,2,4- oxadiazol-3-yl)methoxy)-1H-indol-2- yl)methyl)-1-methylcyclopropane-1- carboxamide |
| 319 | 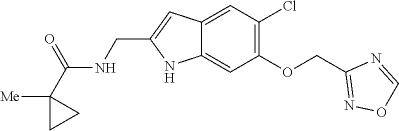 [SMILES: CC1(C(=O)NCc2cc3cc(Cl)c(OCc4ncon4)cc3[nH]2)CC1] | N-((6-((1,2,4-oxadiazol-3-yl)methoxy)- 5-chloro-1H-indol-2-yl)methyl)-1- methylcyclopropane-1-carboxamide |
| 320 | 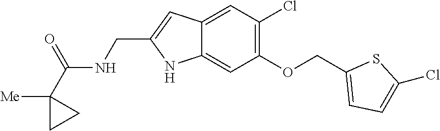 [SMILES: CC1(C(=O)NCc2cc3cc(Cl)c(OCc4ccc(Cl)s4)cc3[nH]2)CC1] | N-((5-chloro-6-((5-chlorothiophen-2- yl)methoxy)-1H-indol-2-yl)methyl)-1- methylcyclopropane-1-carboxamide |
| 321 | 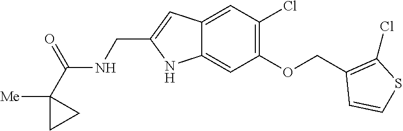 [SMILES: CC1(C(=O)NCc2cc3cc(Cl)c(OCc4ccsc4Cl)cc3[nH]2)CC1] | N-((5-chloro-6-((2-chlorothiophen-3- yl)methoxy)-1H-indol-2-yl)methyl)-1- methylcyclopropane-1-carboxamide |
| 322 | 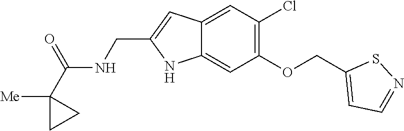 [SMILES: CC1(C(=O)NCc2cc3cc(Cl)c(OCc4ccns4)cc3[nH]2)CC1] | N-((5-chloro-6-(isothiazol-5- ylmethoxy)-1H-indol-2-yl)methyl)-1- methylcyclopropane-1-carboxamide |
| 323 | 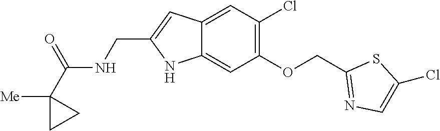 [SMILES: CC1(C(=O)NCc2cc3cc(Cl)c(OCc4ncc(Cl)s4)cc3[nH]2)CC1] | N-((5-chloro-6-((5-chlorothiazol-2- yl)methoxy)-1H-indol-2-yl)methyl)-1- methylcyclopropane-1-carboxamide |
| 324 | 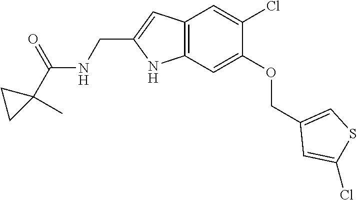 [SMILES: CC1(C(=O)NCc2cc3cc(Cl)c(OCc4csc(Cl)c4)cc3[nH]2)CC1] | N-((5-chloro-6-((5-chlorothiophen-3- yl)methoxy)-1H-indol-2-yl)methyl)-1- methylcyclopropane-1-carboxamide |
| 325 | 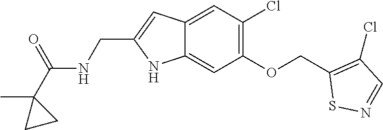 [SMILES: CC1(C(=O)NCc2cc3cc(Cl)c(OCc4sncc4Cl)cc3[nH]2)CC1] | N-((5-chloro-6-((4-chloroisothiazol-5- yl)methoxy)-1H-indol-2-yl)methyl)-1- methylcyclopropane-1-carboxamide |
| 326 | 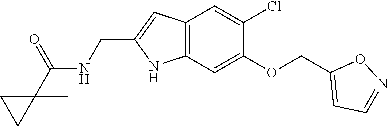 [SMILES: CC1(C(=O)NCc2cc3cc(Cl)c(OCc4ccno4)cc3[nH]2)CC1] | N-((5-chloro-6-(isoxazol-5-ylmethoxy)- 1H-indol-2-yl)methyl)-1- methylcyclopropane-1-carboxamide |
| 327 | 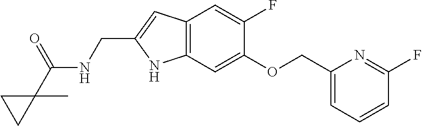 [SMILES: CC1(C(=O)NCc2cc3cc(F)c(OCc4cccc(F)n4)cc3[nH]2)CC1] | N-((5-fluoro-6-((6-fluoropyridin-2- yl)methoxy)-1H-indol-2-yl)methyl)-1- methylcyclopropane-1-carboxamide |
| 328 | 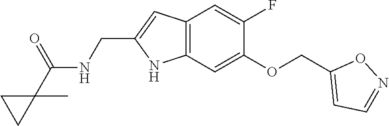 [SMILES: CC1(C(=O)NCc2cc3cc(F)c(OCc4ccno4)cc3[nH]2)CC1] | N-((5-fluoro-6-(isoxazol-5-ylmethoxy)- 1H-indol-2-yl)methyl)-1- methylcyclopropane-1-carboxamide |
| 329 | 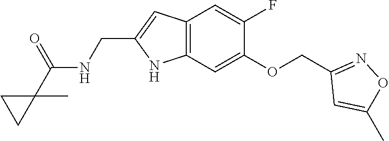 [SMILES: Cc1cc(COc2cc3[nH]c(CNC(=O)C4(C)CC4)cc3cc2F)no1] | N-((5-fluoro-6-((5-methylisoxazol-3- yl)methoxy)-1H-indol-2-yl)methyl)-1- methylcyclopropane-1-carboxamide |
| 330 | 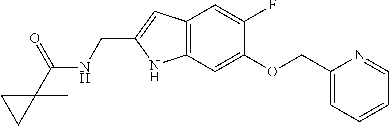 [SMILES: CC1(C(=O)NCc2cc3cc(F)c(OCc4ccccn4)cc3[nH]2)CC1] | N-((5-fluoro-6-(pyridin-2-ylmethoxy)- 1H-indol-2-yl)methyl)-1- methylcyclopropane-1-carboxamide |
| 331 | 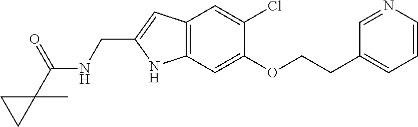 [SMILES: CC1(C(=O)NCc2cc3cc(Cl)c(OCCc4cccnc4)cc3[nH]2)CC1] | N-((5-chloro-6-(2-(pyridin-3- yl)ethoxy)-1H-indol-2-yl)methyl)-1- methylcyclopropane-1-carboxamide |
| 332 | 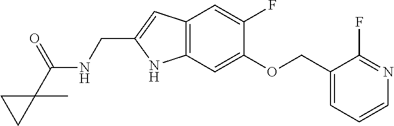 [SMILES: CC1(C(=O)NCc2cc3cc(F)c(OCc4cccnc4F)cc3[nH]2)CC1] | N-((5-fluoro-6-((2-fluoropyridin-3- yl)methoxy)-1H-indol-2-yl)methyl)-1- methylcyclopropane-1-carboxamide |
| 333 | 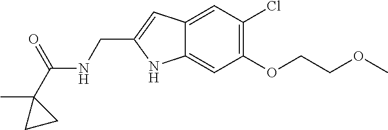 | N-((5-chloro-6-(2-methoxyethoxy)-1H- indol-2-yl)methyl)-1- methylcyclopropane-1-carboxamide |
| 334 | 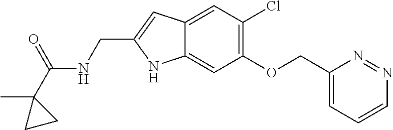 [SMILES: CC1(C(=O)NCc2cc3cc(Cl)c(OCc4cccnn4)cc3[nH]2)CC1] | N-((5-chloro-6-(pyridazin-3- ylmethoxy)-1H-indol-2-yl)methyl)-1- methylcyclopropane-1-carboxamide |
| 335 | 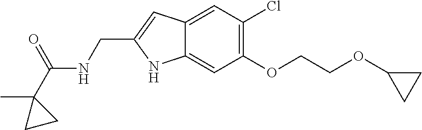 [SMILES: CC1(C(=O)NCc2cc3cc(Cl)c(OCCOC4CC4)cc3[nH]2)CC1] | N-((5-chloro-6-(2- cyclopropoxyethoxy)-1H-indol-2- yl)methyl)-1-methylcyclopropane-1- carboxamide |
| 336 | 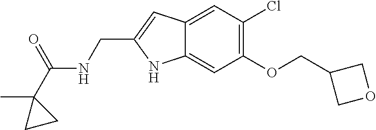 [SMILES: CC1(C(=O)NCc2cc3cc(Cl)c(OCC4COC4)cc3[nH]2)CC1] | N-((5-chloro-6-(oxetan-3-ylmethoxy)- 1H-indol-2-yl)methyl)-1- methylcyclopropane-1-carboxamide |
| 337 | 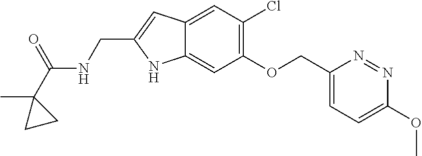 | N-((5-chloro-6-((6-methoxypyridazin- 3-yl)methoxy)-1H-indol-2-yl)methyl)- 1-methylcyclopropane-1-carboxamide |
| 338 | 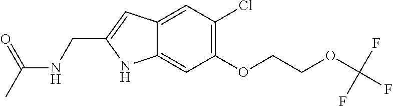 [SMILES: CC(=O)NCc1cc2cc(Cl)c(OCCOC(F)(F)F)cc2[nH]1] | N-((5-chloro-6-(2- (trifluoromethoxy)ethoxy)-1H-indol-2- yl)methyl)acetamide |
| 339 | 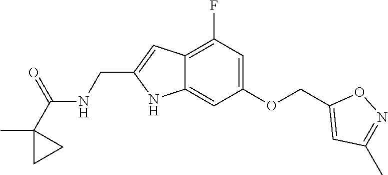 | N-((4-fluoro-6-((3-methylisoxazol-5- yl)methoxy)-1H-indol-2-yl)methyl)-1- methylcyclopropane-1-carboxamide |
| 340 | 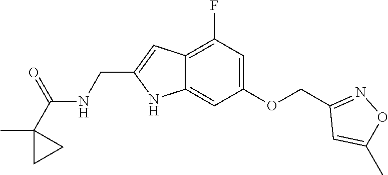 | N-((4-fluoro-6-((5-methylisoxazol-3- yl)methoxy)-1H-indol-2-yl)methyl)-1- methylcyclopropane-1-carboxamide |
| 341 | 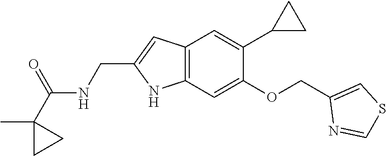 [SMILES: CC1(C(=O)NCc2cc3cc(C4CC4)c(OCc4cscn4)cc3[nH]2)CC1] | N-((5-cyclopropyl-6-(thiazol-4- ylmethoxy)-1H-indol-2-yl)methyl)-1- methylcyclopropane-1-carboxamide |
| 342 | 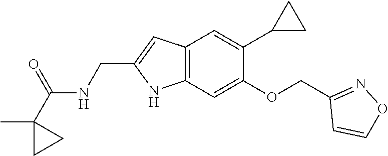 [SMILES: CC1(C(=O)NCc2cc3cc(C4CC4)c(OCc4ccon4)cc3[nH]2)CC1] | N-((5-cyclopropyl-6-(isoxazol-3- ylmethoxy)-1H-indol-2-yl)methyl)-1- methylcyclopropane-1-carboxamide |
| 343 | 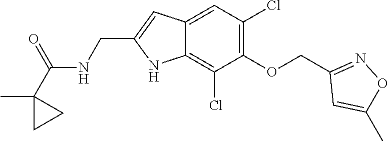 | N-((5,7-dichloro-6-((5-methylisoxazol- 3-yl)methoxy)-1H-indol-2-yl)methyl)- 1-methylcyclopropane-1-carboxamide |
| 344 | 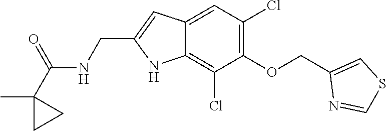 [SMILES: CC1(C(=O)NCc2cc3cc(Cl)c(OCc4cscn4)c(Cl)c3[nH]2)CC1] | N-((5,7-dichloro-6-(thiazol-4- ylmethoxy)-1H-indol-2-yl)methyl)-1- methylcyclopropane-1-carboxamide |
| 345 | 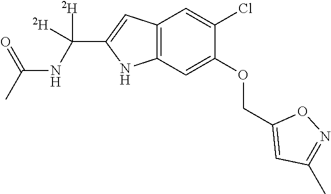 [SMILES: [2H]C([2H])(NC(C)=O)c1cc2cc(Cl)c(OCc3cc(C)no3)cc2[nH]1] | N-((5-chloro-6-((3-methylisoxazol-5- yl)methoxy)-1H-indol-2-yl)methyl- d2)acetamide |
| 346 | 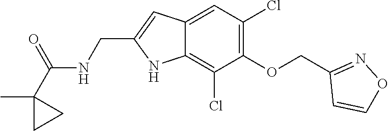 [SMILES: CC1(C(=O)NCc2cc3cc(Cl)c(OCc4ccon4)c(Cl)c3[nH]2)CC1] | N-((5,7-dichloro-6-(isoxazol-3- ylmethoxy)-1H-indol-2-yl)methyl)-1- methylcyclopropane-1-carboxamide |
| 347 | 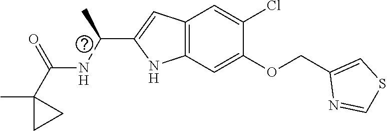 [SMILES: C[C@H](NC(=O)C1(C)CC1)c1cc2cc(Cl)c(OCc3cscn3)cc2[nH]1] | (S)-N-(1-(5-chloro-6-(thiazol-4- ylmethoxy)-1H-indol-2-yl)ethyl)-1- methylcyclopropane-1-carboxamide and (R)-N-(1-(5-chloro-6-(thiazol-4- ylmethoxy)-1H-indol-2-yl)ethyl)-1- methylcyclopropane-1-carboxamide |
| 348 | 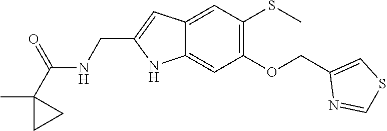 [SMILES: CSc1cc2cc(CNC(=O)C3(C)CC3)[nH]c2cc1OCc1cscn1] | 1-methyl-N-((5-(methylthio)-6-(thiazol- 4-ylmethoxy)-1H-indol-2- yl)methyl)cyclopropane-1-carboxamide |
| 349 | 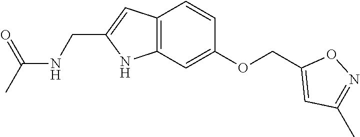 [SMILES: CC(=O)NCc1cc2ccc(OCc3cc(C)no3)cc2[nH]1] | N-((6-((3-methylisoxazol-5- yl)methoxy)-1H-indol-2- yl)methyl)acetamide |
| 350 | 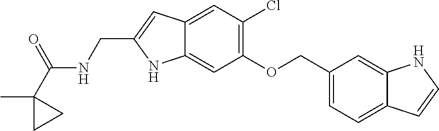 [SMILES: CC1(C(=O)NCc2cc3cc(Cl)c(OCc4ccc5cc[nH]c5c4)cc3[nH]2)CC1] | N-((6-((1H-indol-6-yl)methoxy)-5- chloro-1H-indol-2-yl)methyl)-1- methylcyclopropane-1-carboxamide |
| 351 | 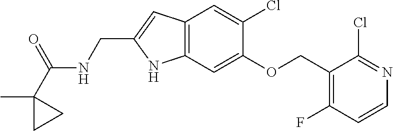 [SMILES: CC1(C(=O)NCc2cc3cc(Cl)c(OCc4c(F)ccnc4Cl)cc3[nH]2)CC1] | N-((5-chloro-6-((2-chloro-4- fluoropyridin-3-yl)methoxy)-1H-indol- 2-yl)methyl)-1-methylcyclopropane-1- carboxamide |
| 352 | 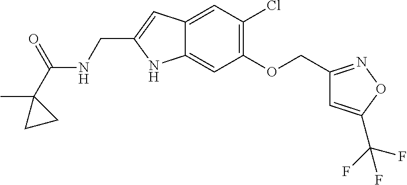 [SMILES: CC1(C(=O)NCc2cc3cc(Cl)c(OCc4cc(C(F)(F)F)on4)cc3[nH]2)CC1] | N-((5-chloro-6-((5- (trifluoromethyl)isoxazol-3- yl)methoxy)-1H-indol-2-yl)methyl)-1- methylcyclopropane-1-carboxamide |
| 353 | 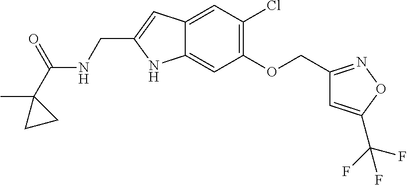 [SMILES: CC1(C(=O)NCc2cc3cc(Cl)c(OCc4cc(C(F)(F)F)on4)cc3[nH]2)CC1] | N-((5-chloro-6-((3,3- difluorocyclobutyl)methoxy)-1H-indol- 2-yl)methyl)-1-methylcyclopropane-1- carboxamide |
| 354 | 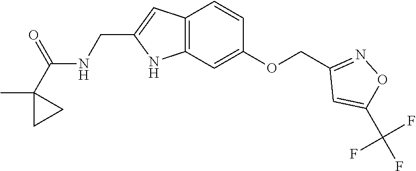 [SMILES: CC1(C(=O)NCc2cc3ccc(OCc4cc(C(F)(F)F)on4)cc3[nH]2)CC1] | 1-methyl-N-((6-((5- (trifluoromethyl)isoxazol-3- yl)methoxy)-1H-indol-2- yl)methyl)cyclopropane-1-carboxamide |
| 355 | 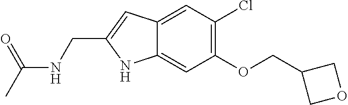 [SMILES: CC(=O)NCc1cc2cc(Cl)c(OCC3COC3)cc2[nH]1] | N-((5-chloro-6-(oxetan-3-ylmethoxy)- 1H-indol-2-yl)methyl)acetamide |
| 356 | 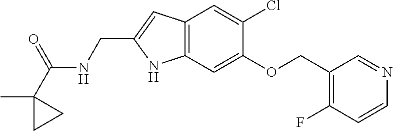 [SMILES: CC1(C(=O)NCc2cc3cc(Cl)c(OCc4cnccc4F)cc3[nH]2)CC1] | N-((5-chloro-6-((4-fluoropyridin-3- yl)methoxy)-1H-indol-2-yl)methyl)-1- methylcyclopropane-1-carboxamide |
| 357 | 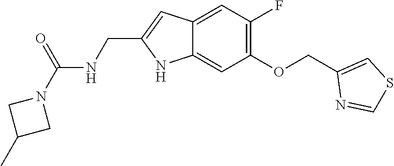 [SMILES: CC1CN(C(=O)NCc2cc3cc(F)c(OCc4cscn4)cc3[nH]2)C1] | N-((5-fluoro-6-(thiazol-4-ylmethoxy)- 1H-indol-2-yl)methyl)-3- methylazetidine-1-carboxamide |
| 358 | 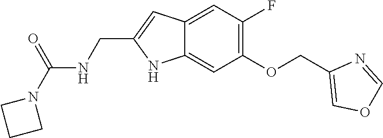 [SMILES: O=C(NCc1cc2cc(F)c(OCc3cocn3)cc2[nH]1)N1CCC1] | N-((5-fluoro-6-(oxazol-4-ylmethoxy)- 1H-indol-2-yl)methyl)azetidine-1- carboxamide |
| 359 | 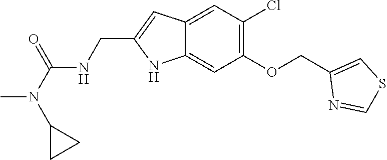 [SMILES: CN(C(=O)NCc1cc2cc(Cl)c(OCc3cscn3)cc2[nH]1)C1CC1] | 3-((5-chloro-6-(thiazol-4-ylmethoxy)- 1H-indol-2-yl)methyl)-1-cyclopropyl- 1-methylurea |
| 360 | 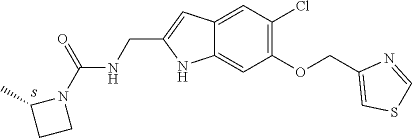 | (S)-N-((5-chloro-6-(thiazol-4- ylmethoxy)-1H-indol-2-yl)methyl)-2- methylazetidine-1-carboxamide |
| 361 | 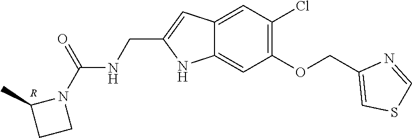 | (R)-N-((5-chloro-6-(thiazol-4- ylmethoxy)-1H-indol-2-yl)methyl)-2- methylazetidine-1-carboxamide |
| 362 | 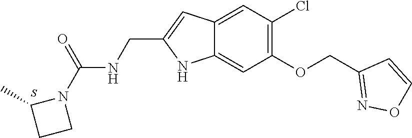 | (S)-N-((5-chloro-6-(isoxazol-3- ylmethoxy)-1H-indol-2-yl)methyl)-2- methylazetidine-1-carboxamide |
| 363 | 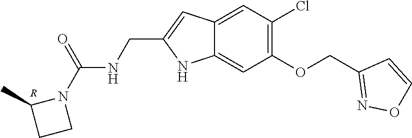 | (R)-N-((5-chloro-6-(isoxazol-3- ylmethoxy)-1H-indol-2-yl)methyl)-2- methylazetidine-1-carboxamide |
| 364 | 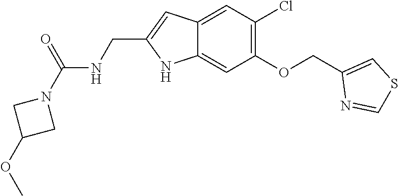 [SMILES: COC1CN(C(=O)NCc2cc3cc(Cl)c(OCc4cscn4)cc3[nH]2)C1] | N-((5-chloro-6-(thiazol-4-ylmethoxy)- 1H-indol-2-yl)methyl)-3- methoxyazetidine-1-carboxamide |
| 365 | 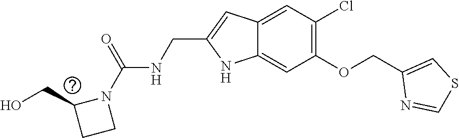 [SMILES: O=C(NCc1cc2cc(Cl)c(OCc3cscn3)cc2[nH]1)N1CC[C@H]1CO] | (S)-N-((5-chloro-6-(thiazol-4- ylmethoxy)-1H-indol-2-yl)methyl)-2- (hydroxymethyl)azetidine-1- carboxamide and (R)-N-((5-chloro-6- (thiazol-4-ylmethoxy)-1H-indol-2- yl)methyl)-2- (hydroxymethyl)azetidine-1- carboxamide |
| 366 | 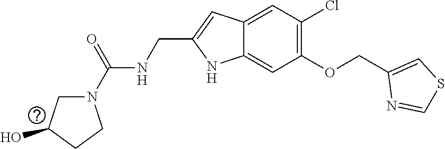 [SMILES: O=C(NCc1cc2cc(Cl)c(OCc3cscn3)cc2[nH]1)N1CC[C@@H](O)C1] | (R)-N-((5-chloro-6-(thiazol-4- ylmethoxy)-1H-indol-2-yl)methyl)-3- hydroxypyrrolidine-1-carboxamide and (S)-N-((5-chloro-6-(thiazol-4- ylmethoxy)-1H-indol-2-yl)methyl)-3- hydroxypyrrolidine-1-carboxamide |
| 367 | 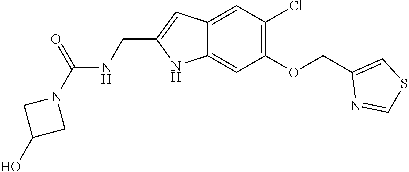 [SMILES: O=C(NCc1cc2cc(Cl)c(OCc3cscn3)cc2[nH]1)N1CC(O)C1] | N-((5-chloro-6-(thiazol-4-ylmethoxy)- 1H-indol-2-yl)methyl)-3- hydroxyazetidine-1-carboxamide |
| 368 | 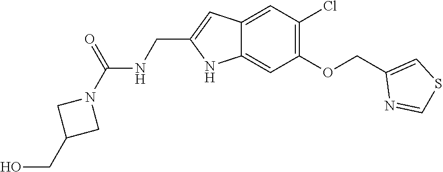 [SMILES: O=C(NCc1cc2cc(Cl)c(OCc3cscn3)cc2[nH]1)N1CC(CO)C1] | N-((5-chloro-6-(thiazol-4-ylmethoxy)- 1H-indol-2-yl)methyl)-3- (hydroxymethyl)azetidine-1- carboxamide |
| 369 | 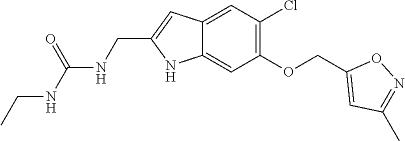 [SMILES: CCNC(=O)NCc1cc2cc(Cl)c(OCc3cc(C)no3)cc2[nH]1] | 1-((5-chloro-6-((3-methylisoxazol-5- yl)methoxy)-1H-indol-2-yl)methyl)-3- ethylurea |
| 370 | 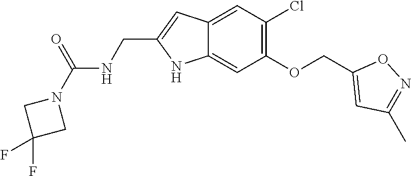 [SMILES: Cc1cc(COc2cc3[nH]c(CNC(=O)N4CC(F)(F)C4)cc3cc2Cl)on1] | N-((5-chloro-6-((3-methylisoxazol-5- yl)methoxy)-1H-indol-2-yl)methyl)- 3,3-difluoroazetidine-1-carboxamide |
| 371 | 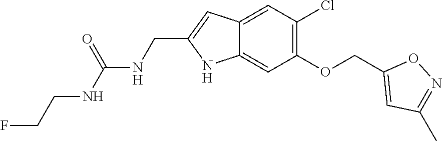 [SMILES: Cc1cc(COc2cc3[nH]c(CNC(=O)NCCF)cc3cc2Cl)on1] | 1-((5-chloro-6-((3-methylisoxazol-5- yl)methoxy)-1H-indol-2-yl)methyl)-3- (2-fluoroethyl)urea |
| 372 | 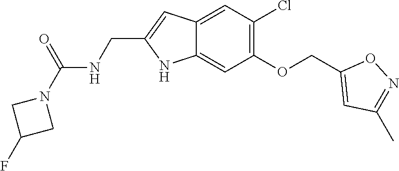 [SMILES: Cc1cc(COc2cc3[nH]c(CNC(=O)N4CC(F)C4)cc3cc2Cl)on1] | N-((5-chloro-6-((3-methylisoxazol-5- yl)methoxy)-1H-indol-2-yl)methyl)-3- fluoroazetidine-1-carboxamide |
| 373 | 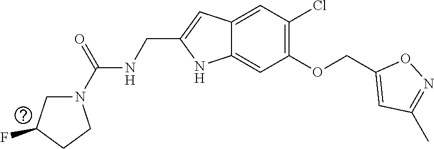 [SMILES: Cc1cc(COc2cc3[nH]c(CNC(=O)N4CC[C@@H](F)C4)cc3cc2Cl)on1] | (R)-N-((5-chloro-6-((3-methylisoxazol- 5-yl)methoxy)-1H-indol-2-yl)methyl)- 3-fluoropyrrolidine-1-carboxamide and (S)-N-((5-chloro-6-((3-methylisoxazol- 5-yl)methoxy)-1H-indol-2-yl)methyl)- 3-fluoropyrrolidine-1-carboxamide |
| 374 | 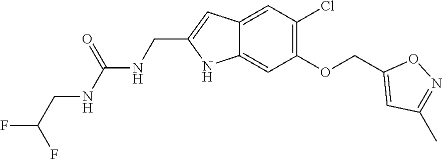 [SMILES: Cc1cc(COc2cc3[nH]c(CNC(=O)NCC(F)F)cc3cc2Cl)on1] | 1-((5-chloro-6-((3-methylisoxazol-5- yl)methoxy)-1H-indol-2-yl)methyl)-3- (2,2-difluoroethyl)urea |
| 375 | 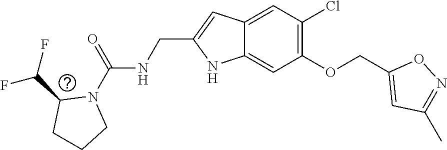 [SMILES: Cc1cc(COc2cc3[nH]c(CNC(=O)N4CCC[C@H]4C(F)F)cc3cc2Cl)on1] | (S)-N-((5-chloro-6-((3-methylisoxazol- 5-yl)methoxy)-1H-indol-2-yl)methyl)- 2-(difluoromethyl)pyrrolidine-1- carboxamide and (R)-N-((5-chloro-6- ((3-methylisoxazol-5-yl)methoxy)-1H- indol-2-yl)methyl)-2- (difluoromethyl)pyrrolidine-1- carboxamide |
| 376 | 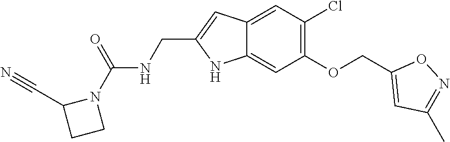 [SMILES: Cc1cc(COc2cc3[nH]c(CNC(=O)N4CCC4C#N)cc3cc2Cl)on1] | N-((5-chloro-6-((3-methylisoxazol-5- yl)methoxy)-1H-indol-2-yl)methyl)-2- cyanoazetidine-1-carboxamide |
| 377 | 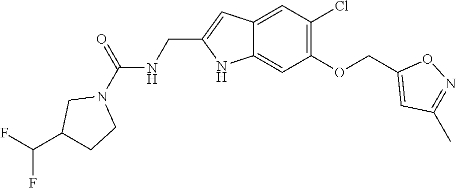 [SMILES: Cc1cc(COc2cc3[nH]c(CNC(=O)N4CCC(C(F)F)C4)cc3cc2Cl)on1] | N-((5-chloro-6-((3-methylisoxazol-5- yl)methoxy)-1H-indol-2-yl)methyl)-3- (difluoromethyl)pyrrolidine-1- carboxamide |
| 378 | 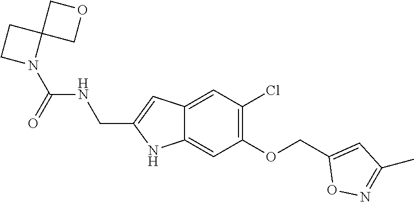 [SMILES: Cc1cc(COc2cc3[nH]c(CNC(=O)N4CCC45COC5)cc3cc2Cl)on1] | N-((5-chloro-6-((3-methylisoxazol-5- yl)methoxy)-1H-indol-2-yl)methyl)-6- oxa-1-azaspiro[3.3]heptane-1- carboxamide |
| 379 | 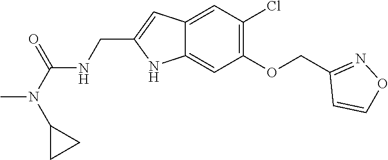 [SMILES: CN(C(=O)NCc1cc2cc(Cl)c(OCc3ccon3)cc2[nH]1)C1CC1] | 3-((5-chloro-6-(isoxazol-3-ylmethoxy)- 1H-indol-2-yl)methyl)-1-cyclopropyl- 1-methylurea |
| 380 | 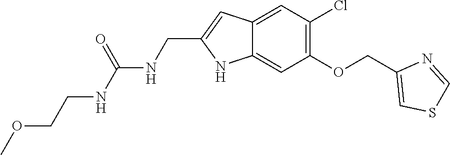 [SMILES: COCCNC(=O)NCc1cc2cc(Cl)c(OCc3cscn3)cc2[nH]1] | 1-((5-chloro-6-(thiazol-4-ylmethoxy)- 1H-indol-2-yl)methyl)-3-(2- methoxyethyl)urea |
| 381 | 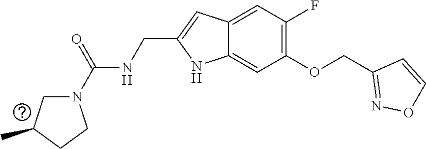 [SMILES: C[C@@H]1CCN(C(=O)NCc2cc3cc(F)c(OCc4ccon4)cc3[nH]2)C1] | (R)-N-((5-fluoro-6-(isoxazol-3- ylmethoxy)-1H-indol-2-yl)methyl)-3- methylpyrrolidine-1-carboxamide |
| 382 | 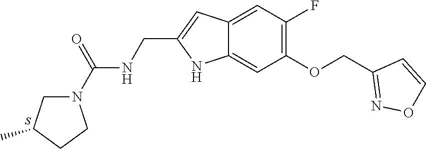 | (S)-N-((5-fluoro-6-(isoxazol-3- ylmethoxy)-1H-indol-2-yl)methyl)-3- methylpyrrolidine-1-carboxamide |
| 383 | 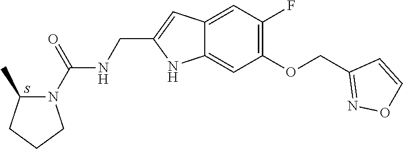 | (R)-N-((5-fluoro-6-(isoxazol-3- ylmethoxy)-1H-indol-2-yl)methyl)-2- methylpyrrolidine-1-carboxamide |
| 384 | 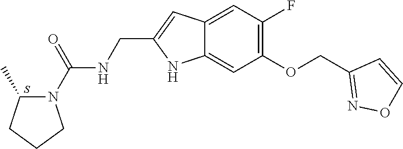 | (S)-N-((5-fluoro-6-(isoxazol-3- ylmethoxy)-1H-indol-2-yl)methyl)-2- methylpyrrolidine-1-carboxamide |
| 385 | 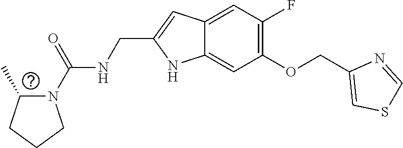 [SMILES: C[C@H]1CCCN1C(=O)NCc1cc2cc(F)c(OCc3cscn3)cc2[nH]1] | (S)-N-((5-fluoro-6-(thiazol-4- ylmethoxy)-1H-indol-2-yl)methyl)-2- methylpyrrolidine-1-carboxamide |
| 386 | 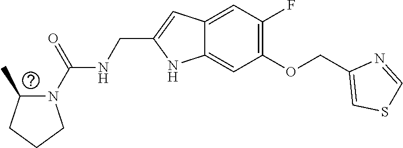 [SMILES: C[C@@H]1CCCN1C(=O)NCc1cc2cc(F)c(OCc3cscn3)cc2[nH]1] | (R)-N-((5-fluoro-6-(thiazol-4- ylmethoxy)-1H-indol-2-yl)methyl)-2- methylpyrrolidine-1-carboxamide |
| 387 | 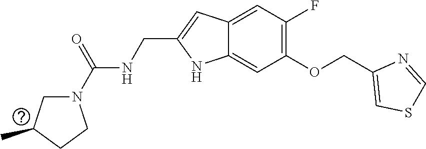 [SMILES: C[C@@H]1CCN(C(=O)NCc2cc3cc(F)c(OCc4cscn4)cc3[nH]2)C1] | (R)-N-((5-fluoro-6-(thiazol-4- ylmethoxy)-1H-indol-2-yl)methyl)-3- methylpyrrolidine-1-carboxamide |
| 388 | 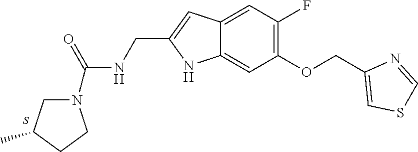 | (S)-N-((5-fluoro-6-(thiazol-4- ylmethoxy)-1H-indol-2-yl)methyl)-3- methylpyrrolidine-1-carboxamide |
| 389 | 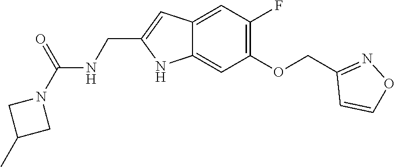 [SMILES: CC1CN(C(=O)NCc2cc3cc(F)c(OCc4ccon4)cc3[nH]2)C1] | N-((5-fluoro-6-(isoxazol-3-ylmethoxy)- 1H-indol-2-yl)methyl)-3- methylazetidine-1-carboxamide |
| 390 | 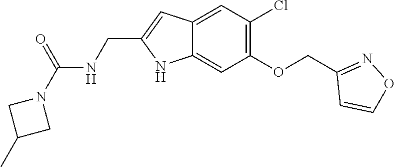 [SMILES: CC1CN(C(=O)NCc2cc3cc(Cl)c(OCc4ccon4)cc3[nH]2)C1] | N-((5-chloro-6-(isoxazol-3-ylmethoxy)- 1H-indol-2-yl)methyl)-3- methylazetidine-1-carboxamide |
| 391 | 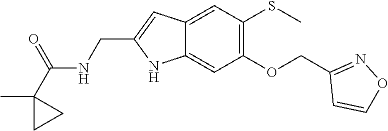 [SMILES: CSc1cc2cc(CNC(=O)C3(C)CC3)[nH]c2cc1OCc1ccon1] | N-((6-(isoxazol-3-ylmethoxy)-5- (methylthio)-1H-indol-2-yl)methyl)-1- methylcyclopropane-1-carboxamide |
| 392 | 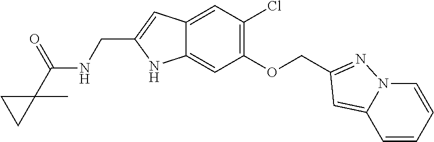 [SMILES: CC1(C(=O)NCc2cc3cc(Cl)c(OCc4cc5ccccn5n4)cc3[nH]2)CC1] | N-((5-chloro-6-(pyrazolo[1,5-a]pyridin- 2-ylmethoxy)-1H-indol-2-yl)methyl)-1- methylcyclopropane-1-carboxamide |
| 393 | 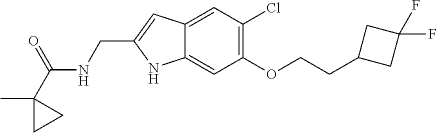 [SMILES: CC1(C(=O)NCc2cc3cc(Cl)c(OCCC4CC(F)(F)C4)cc3[nH]2)CC1] | N-((5-chloro-6-(2-(3,3- difluorocyclobutyl)ethoxy)-1H-indol-2- yl)methyl)-1-methylcyclopropane-1- carboxamide |
| 394 | 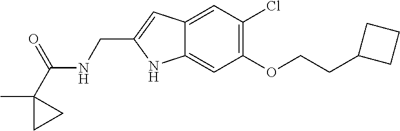 [SMILES: CC1(C(=O)NCc2cc3cc(Cl)c(OCCC4CCC4)cc3[nH]2)CC1] | N-((5-chloro-6-(2-cyclobutylethoxy)- 1H-indol-2-yl)methyl)-1- methylcyclopropane-1-carboxamide |
| 395 | 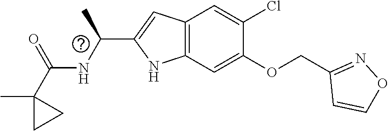 [SMILES: C[C@H](NC(=O)C1(C)CC1)c1cc2cc(Cl)c(OCc3ccon3)cc2[nH]1] | (S)-N-(1-(5-chloro-6-(isoxazol-3- ylmethoxy)-1H-indol-2-yl)ethyl)-1- methylcyclopropane-1-carboxamide and (R)-N-(1-(5-chloro-6-(isoxazol-3- ylmethoxy)-1H-indol-2-yl)ethyl)-1- methylcyclopropane-1-carboxamide |
| 396 | 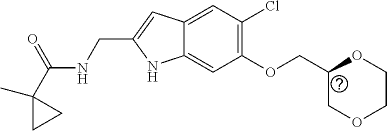 [SMILES: CC1(C(=O)NCc2cc3cc(Cl)c(OC[C@@H]4COCCO4)cc3[nH]2)CC1] | (S)-N-((6-((1,4-dioxan-2-yl)methoxy)- 5-chloro-1H-indol-2-yl)methyl)-1- methylcyclopropane-1-carboxamide and (R)-N-((6-((1,4-dioxan-2- yl)methoxy)-5-chloro-1H-indol-2- yl)methyl)-1-methylcyclopropane-1- carboxamide |
| 397 | 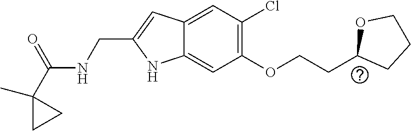 [SMILES: CC1(C(=O)NCc2cc3cc(Cl)c(OCC[C@@H]4CCCO4)cc3[nH]2)CC1] | (S)-N-((5-chloro-6-(2-(tetrahydrofuran- 2-yl)ethoxy)-1H-indol-2-yl)methyl)-1- methylcyclopropane-1-carboxamide and (R)-N-((5-chloro-6-(2- (tetrahydrofuran-2-yl)ethoxy)-1H- indol-2-yl)methyl)-1- methylcyclopropane-1-carboxamide |
| 398 | 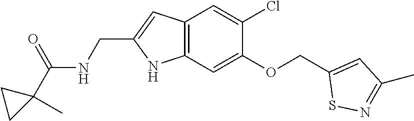 [SMILES: Cc1cc(COc2cc3[nH]c(CNC(=O)C4(C)CC4)cc3cc2Cl)sn1] | N-((5-chloro-6-((3-methylisothiazol-5- yl)methoxy)-1H-indol-2-yl)methyl)-1- methylcyclopropane-1-carboxamide |
| 399 | 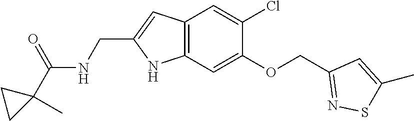 [SMILES: Cc1cc(COc2cc3[nH]c(CNC(=O)C4(C)CC4)cc3cc2Cl)ns1] | N-((5-chloro-6-((5-methylisothiazol-3- yl)methoxy)-1H-indol-2-yl)methyl)-1- methylcyclopropane-1-carboxamide |
| 400 | 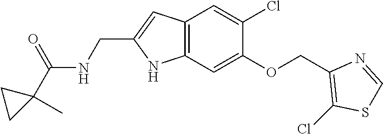 [SMILES: CC1(C(=O)NCc2cc3cc(Cl)c(OCc4ncsc4Cl)cc3[nH]2)CC1] | N-((5-chloro-6-((5-chlorothiazol-4- yl)methoxy)-1H-indol-2-yl)methyl)-1- methylcyclopropane-1-carboxamide |
| 401 | 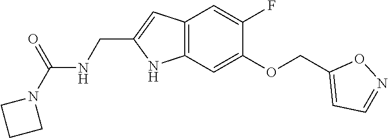 [SMILES: O=C(NCc1cc2cc(F)c(OCc3ccno3)cc2[nH]1)N1CCC1] | N-((5-fluoro-6-(isoxazol-5-ylmethoxy)- 1H-indol-2-yl)methyl)azetidine-1- carboxamide |
| 402 | 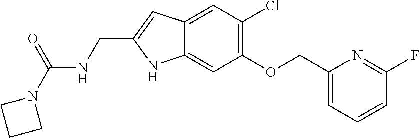 [SMILES: O=C(NCc1cc2cc(Cl)c(OCc3cccc(F)n3)cc2[nH]1)N1CCC1] | N-((5-chloro-6-((6-fluoropyridin-2- yl)methoxy)-1H-indol-2- yl)methyl)azetidine-1-carboxamide |
| 403 | 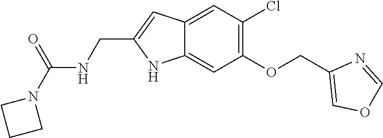 [SMILES: O=C(NCc1cc2cc(Cl)c(OCc3cocn3)cc2[nH]1)N1CCC1] | N-((5-chloro-6-(oxazol-4-ylmethoxy)- 1H-indol-2-yl)methyl)azetidine-1- carboxamide |
| 404 | 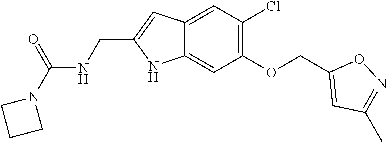 [SMILES: Cc1cc(COc2cc3[nH]c(CNC(=O)N4CCC4)cc3cc2Cl)on1] | N-((5-chloro-6-((3-methylisoxazol-5- yl)methoxy)-1H-indol-2- yl)methyl)azetidine-1-carboxamide |
| 405 | 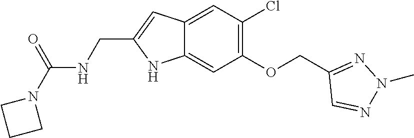 [SMILES: Cn1ncc(COc2cc3[nH]c(CNC(=O)N4CCC4)cc3cc2Cl)n1] | N-((5-chloro-6-((2-methyl-2H-1,2,3- triazol-4-yl)methoxy)-1H-indol-2- yl)methyl)azetidine-1-carboxamide |
| 406 | 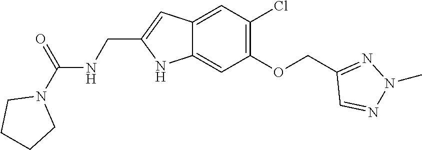 [SMILES: Cn1ncc(COc2cc3[nH]c(CNC(=O)N4CCCC4)cc3cc2Cl)n1] | N-((5-chloro-6-((2-methyl-2H-1,2,3- triazol-4-yl)methoxy)-1H-indol-2- yl)methyl)pyrrolidine-1-carboxamide |
| 407 | 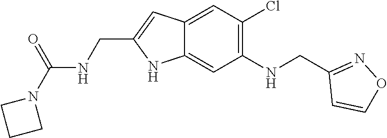 [SMILES: O=C(NCc1cc2cc(Cl)c(NCc3ccon3)cc2[nH]1)N1CCC1] | N-((5-chloro-6-((isoxazol-3- ylmethyl)amino)-1H-indol-2- yl)methyl)azetidine-1-carboxamide |
| 408 | 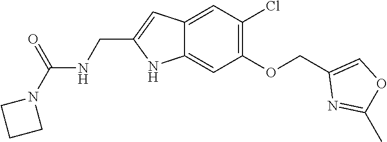 [SMILES: Cc1nc(COc2cc3[nH]c(CNC(=O)N4CCC4)cc3cc2Cl)co1] | N-((5-chloro-6-((2-methyloxazol-4- yl)methoxy)-1H-indol-2- yl)methyl)azetidine-1-carboxamide |
| 409 | 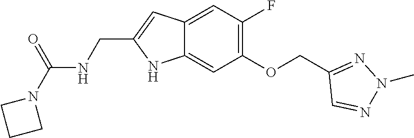 [SMILES: Cn1ncc(COc2cc3[nH]c(CNC(=O)N4CCC4)cc3cc2F)n1] | N-((5-fluoro-6-((2-methyl-2H-1,2,3- triazol-4-yl)methoxy)-1H-indol-2- yl)methyl)azetidine-1-carboxamide |
| 410 | 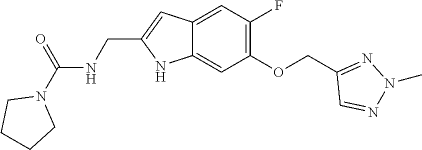 [SMILES: Cn1ncc(COc2cc3[nH]c(CNC(=O)N4CCCC4)cc3cc2F)n1] | N-((5-fluoro-6-((2-methyl-2H-1,2,3- triazol-4-yl)methoxy)-1H-indol-2- yl)methyl)pyrrolidine-1-carboxamide |
| 411 | 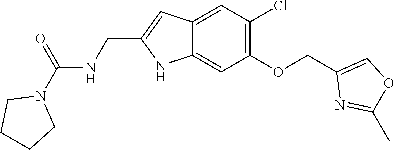 [SMILES: Cc1nc(COc2cc3[nH]c(CNC(=O)N4CCCC4)cc3cc2Cl)co1] | N-((5-chloro-6-((2-methyloxazol-4- yl)methoxy)-1H-indol-2- yl)methyl)pyrrolidine-1-carboxamide |
| 412 | 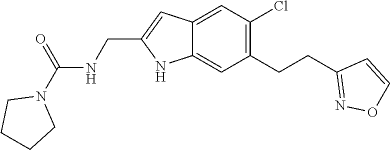 [SMILES: O=C(NCc1cc2cc(Cl)c(CCc3ccon3)cc2[nH]1)N1CCCC1] | N-((5-chloro-6-(2-(isoxazol-3-yl)ethyl)- 1H-indol-2-yl)methyl)pyrrolidine-1- carboxamide |
| 413 | 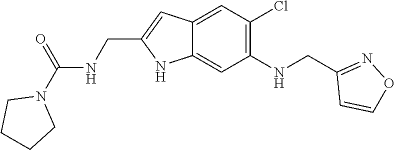 [SMILES: O=C(NCc1cc2cc(Cl)c(NCc3ccon3)cc2[nH]1)N1CCCC1] | N-((5-chloro-6-((isoxazol-3- ylmethyl)amino)-1H-indol-2- yl)methyl)pyrrolidine-1-carboxamide |
| 414 | 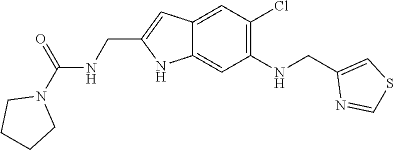 [SMILES: O=C(NCc1cc2cc(Cl)c(NCc3cscn3)cc2[nH]1)N1CCCC1] | N-((5-chloro-6-((thiazol-4- ylmethyl)amino)-1H-indol-2- yl)methyl)pyrrolidine-1-carboxamide |
| 415 | 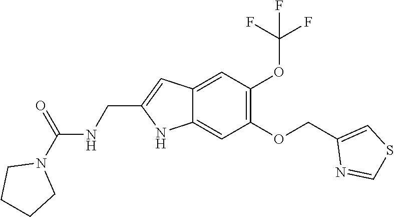 [SMILES: O=C(NCc1cc2cc(OC(F)(F)F)c(OCc3cscn3)cc2[nH]1)N1CCCC1] | N-((6-(thiazol-4-ylmethoxy)-5- (trifluoromethoxy)-1H-indol-2- yl)methyl)pyrrolidine-1-carboxamide |
| 416 | 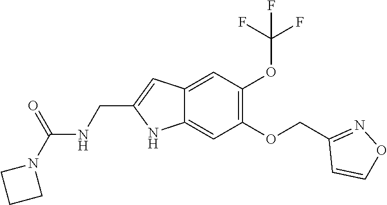 [SMILES: O=C(NCc1cc2cc(OC(F)(F)F)c(OCc3ccon3)cc2[nH]1)N1CCC1] | N-((6-(isoxazol-3-ylmethoxy)-5- (trifluoromethoxy)-1H-indol-2- yl)methyl)azetidine-1-carboxamide |
| 417 | 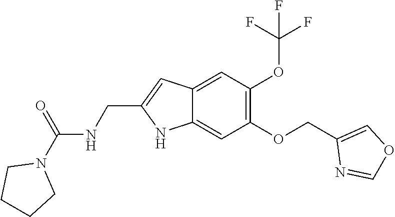 [SMILES: O=C(NCc1cc2cc(OC(F)(F)F)c(OCc3cocn3)cc2[nH]1)N1CCCC1] | N-((6-(oxazol-4-ylmethoxy)-5- (trifluoromethoxy)-1H-indol-2- yl)methyl)pyrrolidine-1-carboxamide |
| 418 | 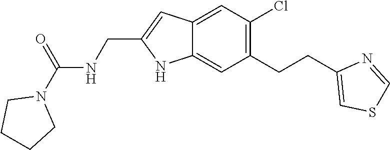 [SMILES: O=C(NCc1cc2cc(Cl)c(CCc3cscn3)cc2[nH]1)N1CCCC1] | N-((5-chloro-6-(2-(thiazol-4-yl)ethyl)- 1H-indol-2-yl)methyl)pyrrolidine-1- carboxamide |
| 419 | 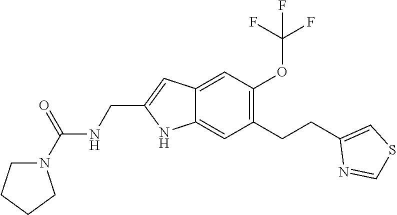 [SMILES: O=C(NCc1cc2cc(OC(F)(F)F)c(CCc3cscn3)cc2[nH]1)N1CCCC1] | N-((6-(2-(thiazol-4-yl)ethyl)-5- (trifluoromethoxy)-1H-indol-2- yl)methyl)pyrrolidine-1-carboxamide |
| 420 | 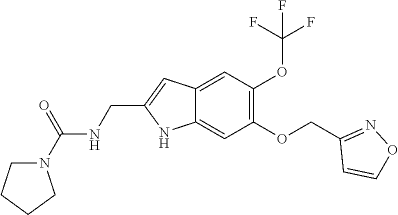 [SMILES: O=C(NCc1cc2cc(OC(F)(F)F)c(OCc3ccon3)cc2[nH]1)N1CCCC1] | N-((6-(isoxazol-3-ylmethoxy)-5- (trifluoromethoxy)-1H-indol-2- yl)methyl)pyrrolidine-1-carboxamide |
| 421 | 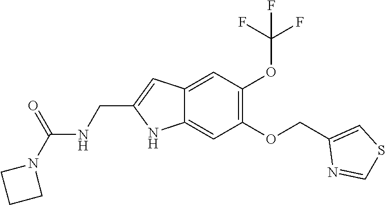 [SMILES: O=C(NCc1cc2cc(OC(F)(F)F)c(OCc3cscn3)cc2[nH]1)N1CCC1] | N-((6-(thiazol-4-ylmethoxy)-5- (trifluoromethoxy)-1H-indol-2- yl)methyl)azetidine-1-carboxamide |
| 422 | 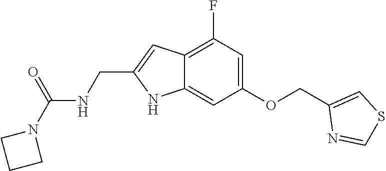 [SMILES: O=C(NCc1cc2c(F)cc(OCc3cscn3)cc2[nH]1)N1CCC1] | N-((4-fluoro-6-(thiazol-4-ylmethoxy)- 1H-indol-2-yl)methyl)azetidine-1- carboxamide |
| 423 | 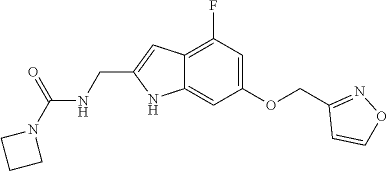 [SMILES: O=C(NCc1cc2c(F)cc(OCc3ccon3)cc2[nH]1)N1CCC1] | N-((4-fluoro-6-(isoxazol-3-ylmethoxy)- 1H-indol-2-yl)methyl)azetidine-1- carboxamide |
| 424 | 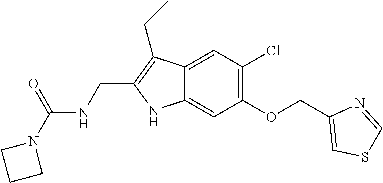 [SMILES: CCc1c(CNC(=O)N2CCC2)[nH]c2cc(OCc3cscn3)c(Cl)cc12] | N-((5-chloro-3-ethyl-6-(thiazol-4- ylmethoxy)-1H-indol-2- yl)methyl)azetidine-1-carboxamide |
| 425 | 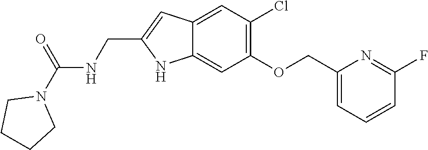 [SMILES: O=C(NCc1cc2cc(Cl)c(OCc3cccc(F)n3)cc2[nH]1)N1CCCC1] | N-((5-chloro-6-((6-fluoropyridin-2- yl)methoxy)-1H-indol-2- yl)methyl)pyrrolidine-1-carboxamide |
| 426 | 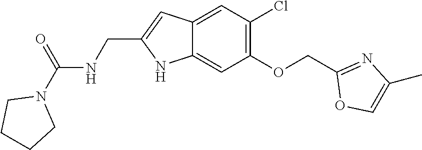 [SMILES: Cc1coc(COc2cc3[nH]c(CNC(=O)N4CCCC4)cc3cc2Cl)n1] | N-((5-chloro-6-((4-methyloxazol-2- yl)methoxy)-1H-indol-2- yl)methyl)pyrrolidine-1-carboxamide |
| 427 | 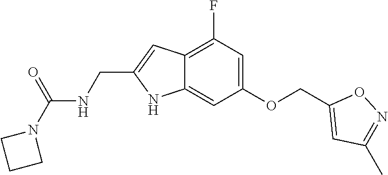 [SMILES: Cc1cc(COc2cc(F)c3cc(CNC(=O)N4CCC4)[nH]c3c2)on1] | N-((4-fluoro-6-((3-methylisoxazol-5- yl)methoxy)-1H-indol-2- yl)methyl)azetidine-1-carboxamide |
| 428 | 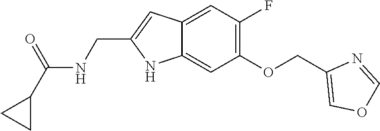 [SMILES: O=C(NCc1cc2cc(F)c(OCc3cocn3)cc2[nH]1)C1CC1] | N-((5-fluoro-6-(oxazol-4-ylmethoxy)- 1H-indol-2- yl)methyl)cyclopropanecarboxamide |
| 429 | 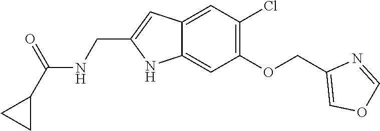 [SMILES: O=C(NCc1cc2cc(Cl)c(OCc3cocn3)cc2[nH]1)C1CC1] | N-((5-chloro-6-(oxazol-4-ylmethoxy)- 1H-indol-2- yl)methyl)cyclopropanecarboxamide |
| 430 | 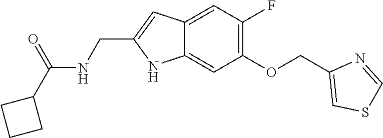 [SMILES: O=C(NCc1cc2cc(F)c(OCc3cscn3)cc2[nH]1)C1CCC1] | N-((5-fluoro-6-(thiazol-4-ylmethoxy)- 1H-indol-2- yl)methyl)cyclobutanecarboxamide |
| 431 | 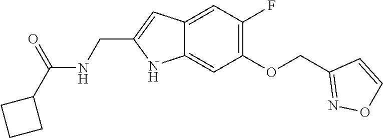 [SMILES: O=C(NCc1cc2cc(F)c(OCc3ccon3)cc2[nH]1)C1CCC1] | N-((5-fluoro-6-(isoxazol-3-ylmethoxy)- 1H-indol-2- yl)methyl)cyclobutanecarboxamide |
| 432 | 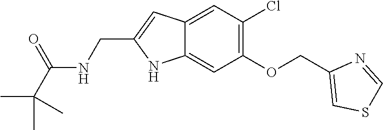 [SMILES: CC(C)(C)C(=O)NCc1cc2cc(Cl)c(OCc3cscn3)cc2[nH]1] | N-((5-chloro-6-(thiazol-4-ylmethoxy)- 1H-indol-2-yl)methyl)pivalamide |
| 433 | 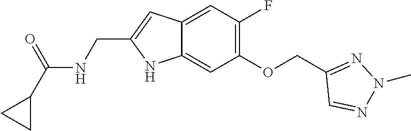 [SMILES: Cn1ncc(COc2cc3[nH]c(CNC(=O)C4CC4)cc3cc2F)n1] | N-((5-fluoro-6-((2-methyl-2H-1,2,3- triazol-4-yl)methoxy)-1H-indol-2- yl)methyl)cyclopropanecarboxamide |
| 434 | 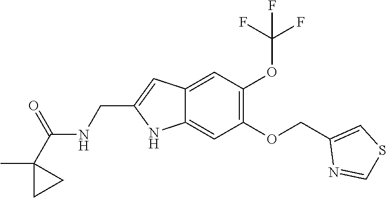 [SMILES: CC1(C(=O)NCc2cc3cc(OC(F)(F)F)c(OCc4cscn4)cc3[nH]2)CC1] | 1-methyl-N-((6-(thiazol-4-ylmethoxy)- 5-(trifluoromethoxy)-1H-indol-2- yl)methyl)cyclopropane-1-carboxamide |
| 435 | 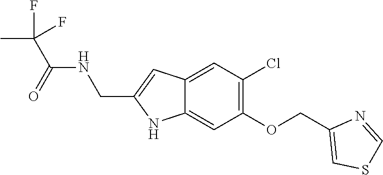 [SMILES: CC(F)(F)C(=O)NCc1cc2cc(Cl)c(OCc3cscn3)cc2[nH]1] | N-((5-chloro-6-(thiazol-4-ylmethoxy)- 1H-indol-2-yl)methyl)-2,2- difluoropropanamide |
| 436 | 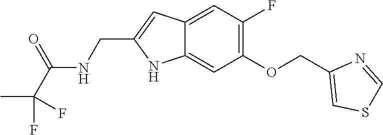 [SMILES: CC(F)(F)C(=O)NCc1cc2cc(F)c(OCc3cscn3)cc2[nH]1] | 2,2-difluoro-N-((5-fluoro-6-(thiazol-4- ylmethoxy)-1H-indol-2- yl)methyl)propanamide |
| 437 | 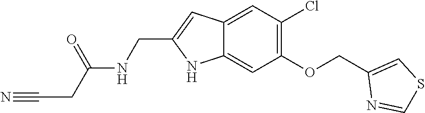 [SMILES: N#CCC(=O)NCc1cc2cc(Cl)c(OCc3cscn3)cc2[nH]1] | N-((5-chloro-6-(thiazol-4-ylmethoxy)- 1H-indol-2-yl)methyl)-2- cyanoacetamide |
| 438 | 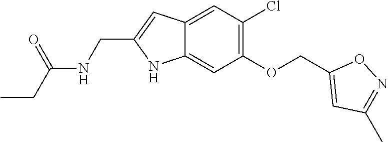 [SMILES: CCC(=O)NCc1cc2cc(Cl)c(OCc3cc(C)no3)cc2[nH]1] | N-((5-chloro-6-((3-methylisoxazol-5- yl)methoxy)-1H-indol-2- yl)methyl)propionamide |
| 439 | 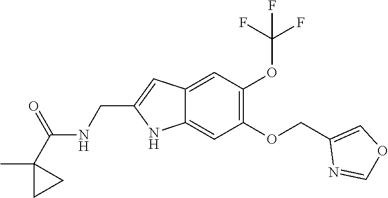 [SMILES: CC1(C(=O)NCc2cc3cc(OC(F)(F)F)c(OCc4cocn4)cc3[nH]2)CC1] | 1-methyl-N-((6-(oxazol-4-ylmethoxy)- 5-(trifluoromethoxy)-1H-indol-2- yl)methyl)cyclopropane-1-carboxamide |
| 440 | 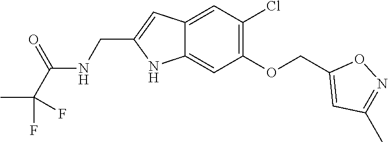 [SMILES: Cc1cc(COc2cc3[nH]c(CNC(=O)C(C)(F)F)cc3cc2Cl)on1] | N-((5-chloro-6-((3-methylisoxazol-5- yl)methoxy)-1H-indol-2-yl)methyl)- 2,2-difluoropropanamide |
| 441 | 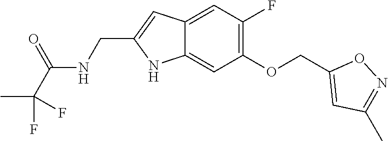 [SMILES: Cc1cc(COc2cc3[nH]c(CNC(=O)C(C)(F)F)cc3cc2F)on1] | 2,2-difluoro-N-((5-fluoro-6-((3- methylisoxazol-5-yl)methoxy)-1H- indol-2-yl)methyl)propanamide |
| 442 | 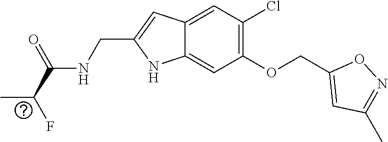 [SMILES: Cc1cc(COc2cc3[nH]c(CNC(=O)[C@H](C)F)cc3cc2Cl)on1] | (S)-N-((5-chloro-6-((3-methylisoxazol- 5-yl)methoxy)-1H-indol-2-yl)methyl)- 2-fluoropropanamide and (R)-N-((5- chloro-6-((3-methylisoxazol-5- yl)methoxy)-1H-indol-2-yl)methyl)-2- fluoropropanamide |
| 443 | 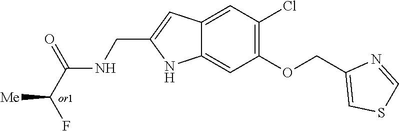 [SMILES: C[C@H](F)C(=O)NCc1cc2cc(Cl)c(OCc3cscn3)cc2[nH]1] | (S or R)-N-((5-chloro-6-(thiazol-4- ylmethoxy)-1H-indol-2-yl)methyl)-2- fluoropropanamide |
| 444 | 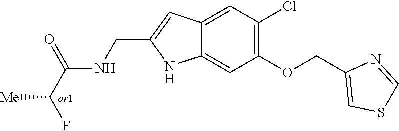 [SMILES: C[C@@H](F)C(=O)NCc1cc2cc(Cl)c(OCc3cscn3)cc2[nH]1] | (R or S)-N-((5-chloro-6-(thiazol-4- ylmethoxy)-1H-indol-2-yl)methyl)-2- fluoropropanamide |
| 445 | 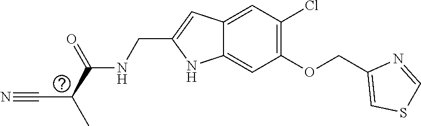 [SMILES: C[C@@H](C#N)C(=O)NCc1cc2cc(Cl)c(OCc3cscn3)cc2[nH]1] | (S)-N-((5-chloro-6-(thiazol-4- ylmethoxy)-1H-indol-2-yl)methyl)-2- cyanopropanamide and (R)-N-((5- chloro-6-(thiazol-4-ylmethoxy)-1H- indol-2-yl)methyl)-2- cyanopropanamide |
| 446 | 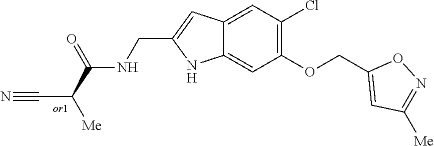 [SMILES: Cc1cc(COc2cc3[nH]c(CNC(=O)[C@@H](C)C#N)cc3cc2Cl)on1] | (R or S)-N-((5-chloro-6-((3- methylisoxazol-5-yl)methoxy)-1H- indol-2-yl)methyl)-2- cyanopropanamide |
| 447 | 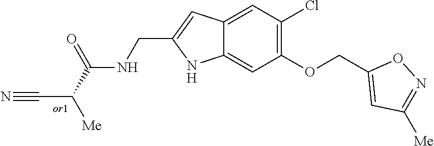 [SMILES: Cc1cc(COc2cc3[nH]c(CNC(=O)[C@H](C)C#N)cc3cc2Cl)on1] | (S or R)-N-((5-chloro-6-((3- methylisoxazol-5-yl)methoxy)-1H- indol-2-yl)methyl)-2- cyanopropanamide |
| 448 | 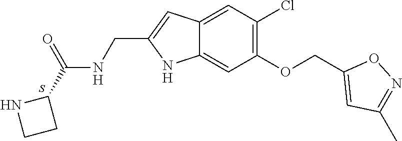 | (S)-N-((5-chloro-6-((3-methylisoxazol- 5-yl)methoxy)-1H-indol-2- yl)methyl)azetidine-2-carboxamide |
| 449 | 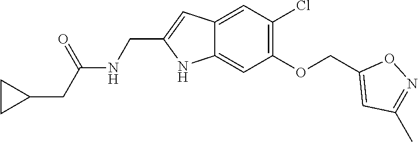 [SMILES: Cc1cc(COc2cc3[nH]c(CNC(=O)CC4CC4)cc3cc2Cl)on1] | N-((5-chloro-6-((3-methylisoxazol-5- yl)methoxy)-1H-indol-2-yl)methyl)-2- cyclopropylacetamide |
| 450 | 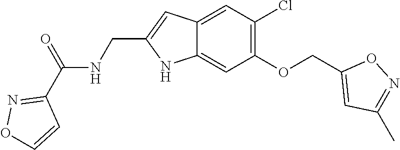 [SMILES: Cc1cc(COc2cc3[nH]c(CNC(=O)c4ccon4)cc3cc2Cl)on1] | N-((5-chloro-6-((3-methylisoxazol-5- yl)methoxy)-1H-indol-2- yl)methyl)isoxazole-3-carboxamide |
| 451 | 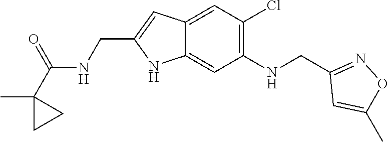 | N-((5-chloro-6-(((5-methylisoxazol-3- yl)methyl)amino)-1H-indol-2- yl)methyl)-1-methylcyclopropane-1- carboxamide |
| 452 | 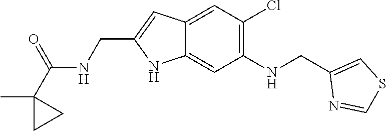 [SMILES: CC1(C(=O)NCc2cc3cc(Cl)c(NCc4cscn4)cc3[nH]2)CC1] | N-((5-chloro-6-((thiazol-4- ylmethyl)amino)-1H-indol-2- yl)methyl)-1-methylcyclopropane-1- carboxamide |
| 453 | 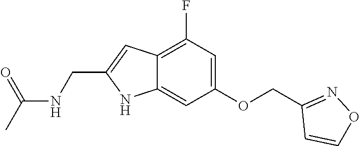 [SMILES: CC(=O)NCc1cc2c(F)cc(OCc3ccon3)cc2[nH]1] | N-((4-fluoro-6-(isoxazol-3-ylmethoxy)- 1H-indol-2-yl)methyl)acetamide |
| 454 | 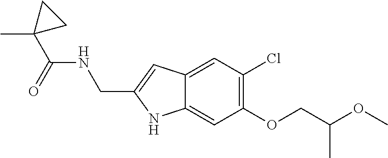 | N-((5-chloro-6-(2-methoxypropoxy)- 1H-indol-2-yl)methyl)-1- methylcyclopropane-1-carboxamide |
| 455 | 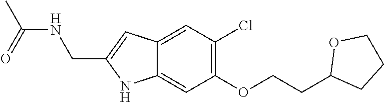 [SMILES: CC(=O)NCc1cc2cc(Cl)c(OCCC3CCCO3)cc2[nH]1] | N-((5-chloro-6-(2-(tetrahydrofuran-2- yl)ethoxy)-1H-indol-2- yl)methyl)acetamide |
| 456 | 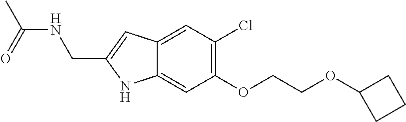 [SMILES: CC(=O)NCc1cc2cc(Cl)c(OCCOC3CCC3)cc2[nH]1] | N-((5-chloro-6-(2-cyclobutoxyethoxy)- 1H-indol-2-yl)methyl)acetamide |
| 457 | 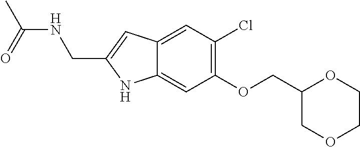 [SMILES: CC(=O)NCc1cc2cc(Cl)c(OCC3COCCO3)cc2[nH]1] | N-((6-((1,4-dioxan-2-yl)methoxy)-5- chloro-1H-indol-2-yl)methyl)acetamide |
| 458 | 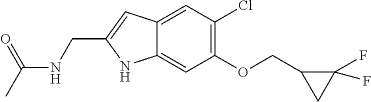 [SMILES: CC(=O)NCc1cc2cc(Cl)c(OCC3CC3(F)F)cc2[nH]1] | N-((5-chloro-6-((2,2- difluorocyclopropyl)methoxy)-1H- indol-2-yl)methyl)acetamide |
| 459 | 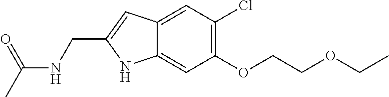 [SMILES: CCOCCOc1cc2[nH]c(CNC(C)=O)cc2cc1Cl] | N-((5-chloro-6-(2-ethoxyethoxy)-1H- indol-2-yl)methyl)acetamide |
| 460 | 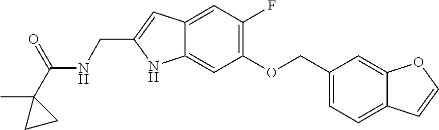 [SMILES: CC1(C(=O)NCc2cc3cc(F)c(OCc4ccc5ccoc5c4)cc3[nH]2)CC1] | N-((6-(benzofuran-6-ylmethoxy)-5- fluoro-1H-indol-2-yl)methyl)-1- methylcyclopropane-1-carboxamide |
| 461 | 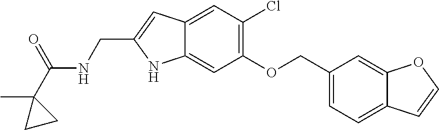 [SMILES: CC1(C(=O)NCc2cc3cc(Cl)c(OCc4ccc5ccoc5c4)cc3[nH]2)CC1] | N-((6-(benzofuran-6-ylmethoxy)-5- chloro-1H-indol-2-yl)methyl)-1- methylcyclopropane-1-carboxamide |
| 462 | 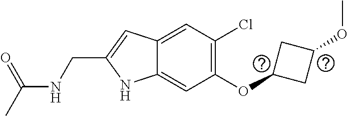 [SMILES: CO[C@H]1C[C@H](Oc2cc3[nH]c(CNC(C)=O)cc3cc2Cl)C1] | N-((5-chloro-6-((trans)-3- methoxycyclobutoxy)-1H-indol-2- yl)methyl)acetamide |
| 463 | 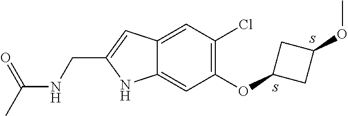 | N-((5-chloro-6-((cis)-3- methoxycyclobutoxy)-1H-indol-2- yl)methyl)acetamide |
| 464 | 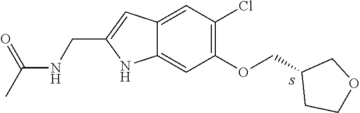 | (S)-N-((5-chloro-6-((tetrahydrofuran-3- yl)methoxy)-1H-indol-2- yl)methyl)acetamide |
| 465 | 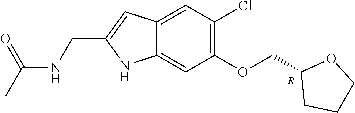 | (R)-N-((5-chloro-6-((tetrahydrofuran-2- yl)methoxy)-1H-indol-2- yl)methyl)acetamide |
| 466 | 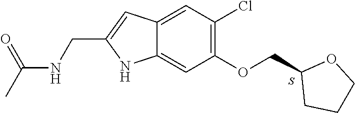 | (S)-N-((5-chloro-6-((tetrahydrofuran-2- yl)methoxy)-1H-indol-2- yl)methyl)acetamide |
| 467 | 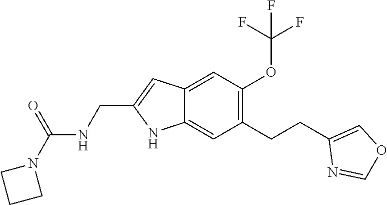 [SMILES: O=C(NCc1cc2cc(OC(F)(F)F)c(CCc3cocn3)cc2[nH]1)N1CCC1] | N-((6-(2-(oxazol-4-yl)ethyl)-5- (trifluoromethoxy)-1H-indol-2- yl)methyl)azetidine-1-carboxamide |
| 468 | 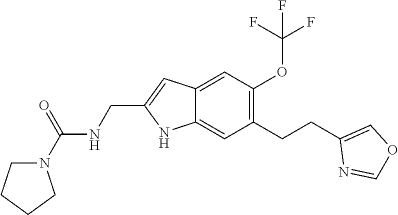 [SMILES: O=C(NCc1cc2cc(OC(F)(F)F)c(CCc3cocn3)cc2[nH]1)N1CCCC1] | N-((6-(2-(oxazol-4-yl)ethyl)-5- (trifluoromethoxy)-1H-indol-2- yl)methyl)pyrrolidine-1-carboxamide |
| 469 | 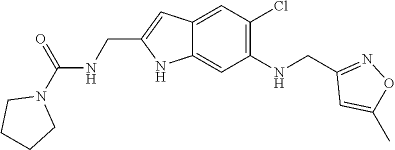 [SMILES: Cc1cc(CNc2cc3[nH]c(CNC(=O)N4CCCC4)cc3cc2Cl)no1] | N-((5-chloro-6-(((5-methylisoxazol-3- yl)methyl)amino)-1H-indol-2- yl)methyl)pyrrolidine-1-carboxamide |
| 470 | 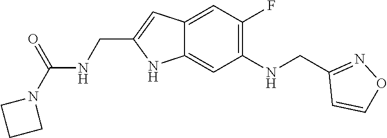 [SMILES: O=C(NCc1cc2cc(F)c(NCc3ccon3)cc2[nH]1)N1CCC1] | N-((5-fluoro-6-((isoxazol-3- ylmethyl)amino)-1H-indol-2- yl)methyl)azetidine-1-carboxamide |
| 471 | 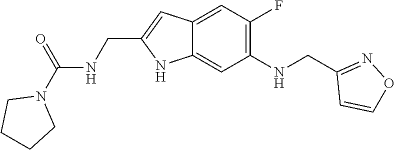 [SMILES: O=C(NCc1cc2cc(F)c(NCc3ccon3)cc2[nH]1)N1CCCC1] | N-((5-fluoro-6-((isoxazol-3- ylmethyl)amino)-1H-indol-2- yl)methyl)pyrrolidine-1-carboxamide |
| 472 | 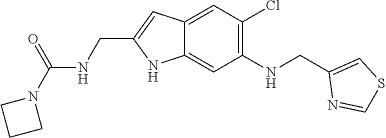 [SMILES: O=C(NCc1cc2cc(Cl)c(NCc3cscn3)cc2[nH]1)N1CCC1] | N-((5-chloro-6-((thiazol-4- ylmethyl)amino)-1H-indol-2- yl)methyl)azetidine-1-carboxamide |
| 473 | 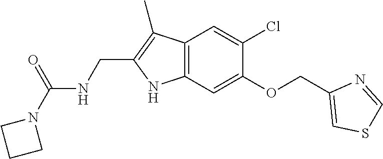 [SMILES: Cc1c(CNC(=O)N2CCC2)[nH]c2cc(OCc3cscn3)c(Cl)cc12] | N-((5-chloro-3-methyl-6-(thiazol-4- ylmethoxy)-1H-indol-2- yl)methyl)azetidine-1-carboxamide |
| 474 | 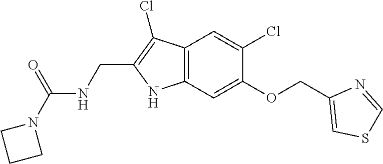 [SMILES: O=C(NCc1[nH]c2cc(OCc3cscn3)c(Cl)cc2c1Cl)N1CCC1] | N-((3,5-dichloro-6-(thiazol-4- ylmethoxy)-1H-indol-2- yl)methyl)azetidine-1-carboxamide |
| 475 | 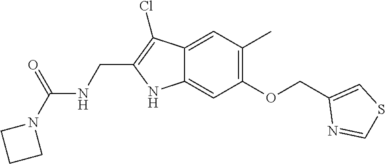 [SMILES: Cc1cc2c(Cl)c(CNC(=O)N3CCC3)[nH]c2cc1OCc1cscn1] | N-((3-chloro-5-methyl-6-(thiazol-4- ylmethoxy)-1H-indol-2- yl)methyl)azetidine-1-carboxamide |
| 476 | 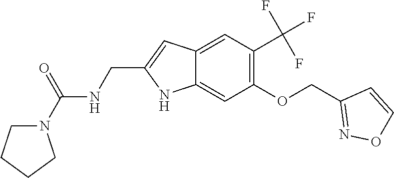 [SMILES: O=C(NCc1cc2cc(C(F)(F)F)c(OCc3ccon3)cc2[nH]1)N1CCCC1] | N-((6-(isoxazol-3-ylmethoxy)-5- (trifluoromethyl)-1H-indol-2- yl)methyl)pyrrolidine-1-carboxamide |
| 477 | 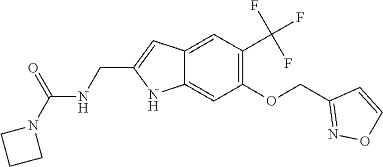 [SMILES: O=C(NCc1cc2cc(C(F)(F)F)c(OCc3ccon3)cc2[nH]1)N1CCC1] | N-((6-(isoxazol-3-ylmethoxy)-5- (trifluoromethyl)-1H-indol-2- yl)methyl)azetidine-1-carboxamide |
| 478 | 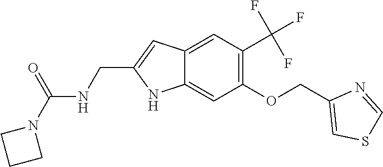 [SMILES: O=C(NCc1cc2cc(C(F)(F)F)c(OCc3cscn3)cc2[nH]1)N1CCC1] | N-((6-(thiazol-4-ylmethoxy)-5- (trifluoromethyl)-1H-indol-2- yl)methyl)azetidine-1-carboxamide |
| 479 | 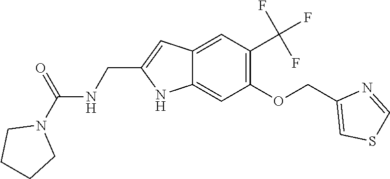 [SMILES: O=C(NCc1cc2cc(C(F)(F)F)c(OCc3cscn3)cc2[nH]1)N1CCCC1] | N-((6-(thiazol-4-ylmethoxy)-5- (trifluoromethyl)-1H-indol-2- yl)methyl)pyrrolidine-1-carboxamide |
| 480 | 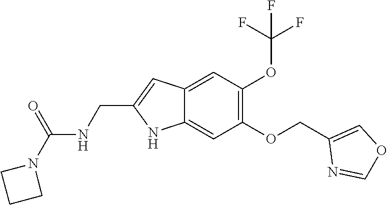 [SMILES: O=C(NCc1cc2cc(OC(F)(F)F)c(OCc3cocn3)cc2[nH]1)N1CCC1] | N-((6-(oxazol-4-ylmethoxy)-5- (trifluoromethoxy)-1H-indol-2- yl)methyl)azetidine-1-carboxamide |
| 481 | 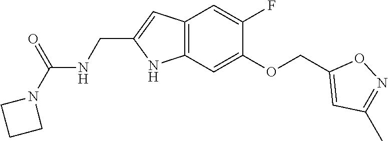 [SMILES: Cc1cc(COc2cc3[nH]c(CNC(=O)N4CCC4)cc3cc2F)on1] | N-((5-fluoro-6-((3-methylisoxazol-5- yl)methoxy)-1H-indol-2- yl)methyl)azetidine-1-carboxamide |
| 482 | 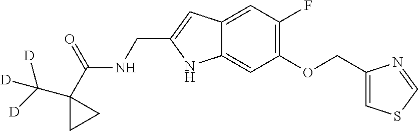 [SMILES: [2H]C([2H])([2H])C1(C(=O)NCc2cc3cc(F)c(OCc4cscn4)cc3[nH]2)CC1] | N-((5-fluoro-6-(thiazol-4-ylmethoxy)- 1H-indol-2-yl)methyl)-1-(methyl- d3)cyclopropane-1-carboxamide |
| 483 | 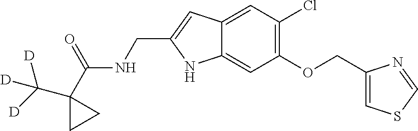 [SMILES: [2H]C([2H])([2H])C1(C(=O)NCc2cc3cc(Cl)c(OCc4cscn4)cc3[nH]2)CC1] | N-((5-chloro-6-(thiazol-4-ylmethoxy)- 1H-indol-2-yl)methyl)-1-(methyl- d3)cyclopropane-1-carboxamide |
| 484 | 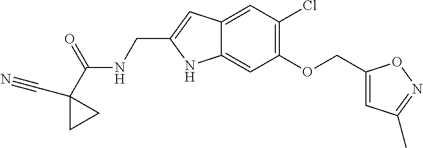 [SMILES: Cc1cc(COc2cc3[nH]c(CNC(=O)C4(C#N)CC4)cc3cc2Cl)on1] | N-((5-chloro-6-((3-methylisoxazol-5- yl)methoxy)-1H-indol-2-yl)methyl)-1- cyanocyclopropane-1-carboxamide |
| 485 | 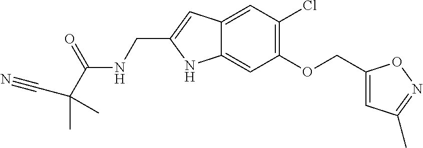 [SMILES: Cc1cc(COc2cc3[nH]c(CNC(=O)C(C)(C)C#N)cc3cc2Cl)on1] | N-((5-chloro-6-((3-methylisoxazol-5- yl)methoxy)-1H-indol-2-yl)methyl)-2- cyano-2-methylpropanamide |
| 486 | 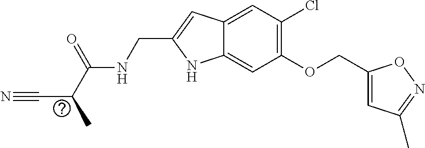 [SMILES: Cc1cc(COc2cc3[nH]c(CNC(=O)[C@@H](C)C#N)cc3cc2Cl)on1] | (S)-N-((5-chloro-6-((3-methylisoxazol- 5-yl)methoxy)-1H-indol-2-yl)methyl)- 2-cyanopropanamide and (R)-N-((5- chloro-6-((3-methylisoxazol-5- yl)methoxy)-1H-indol-2-yl)methyl)-2- cyanopropanamide |
| 487 | 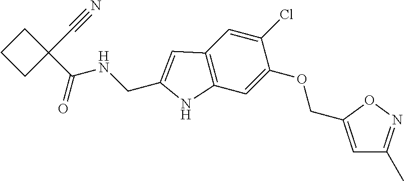 [SMILES: Cc1cc(COc2cc3[nH]c(CNC(=O)C4(C#N)CCC4)cc3cc2Cl)on1] | N-((5-chloro-6-((3-methylisoxazol-5- yl)methoxy)-1H-indol-2-yl)methyl)-1- cyanocyclobutane-1-carboxamide |
| 488 | 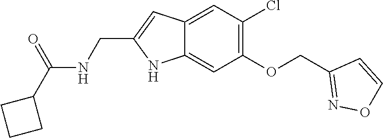 [SMILES: O=C(NCc1cc2cc(Cl)c(OCc3ccon3)cc2[nH]1)C1CCC1] | N-((5-chloro-6-(isoxazol-3-ylmethoxy)- 1H-indol-2- yl)methyl)cyclobutanecarboxamide |
| 489 | 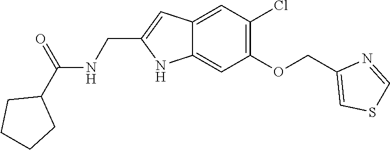 [SMILES: O=C(NCc1cc2cc(Cl)c(OCc3cscn3)cc2[nH]1)C1CCCC1] | N-((5-chloro-6-(thiazol-4-ylmethoxy)- 1H-indol-2- yl)methyl)cyclopentanecarboxamide |
| 490 | 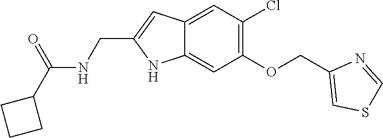 [SMILES: O=C(NCc1cc2cc(Cl)c(OCc3cscn3)cc2[nH]1)C1CCC1] | N-((5-chloro-6-(thiazol-4-ylmethoxy)- 1H-indol-2- yl)methyl)cyclobutanecarboxamide |
| 491 | 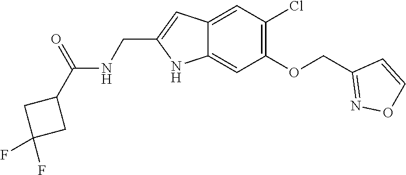 [SMILES: O=C(NCc1cc2cc(Cl)c(OCc3ccon3)cc2[nH]1)C1CC(F)(F)C1] | N-((5-chloro-6-(isoxazol-3-ylmethoxy)- 1H-indol-2-yl)methyl)-3,3- difluorocyclobutane-1-carboxamide |
| 492 | 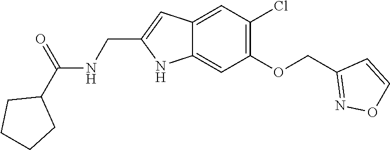 [SMILES: O=C(NCc1cc2cc(Cl)c(OCc3ccon3)cc2[nH]1)C1CCCC1] | N-((5-chloro-6-(isoxazol-3-ylmethoxy)- 1H-indol-2- yl)methyl)cyclopentanecarboxamide |
| 493 | 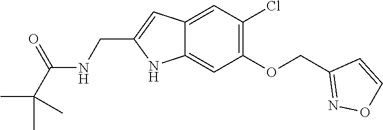 [SMILES: CC(C)(C)C(=O)NCc1cc2cc(Cl)c(OCc3ccon3)cc2[nH]1] | N-((5-chloro-6-(isoxazol-3-ylmethoxy)- 1H-indol-2-yl)methyl)pivalamide |
| 494 | 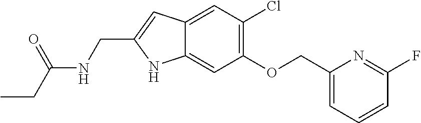 [SMILES: CCC(=O)NCc1cc2cc(Cl)c(OCc3cccc(F)n3)cc2[nH]1] | N-((5-chloro-6-((6-fluoropyridin-2- yl)methoxy)-1H-indol-2- yl)methyl)propionamide |
| 495 | 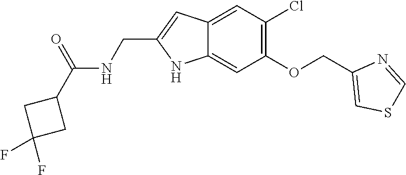 [SMILES: O=C(NCc1cc2cc(Cl)c(OCc3cscn3)cc2[nH]1)C1CC(F)(F)C1] | N-((5-chloro-6-(thiazol-4-ylmethoxy)- 1H-indol-2-yl)methyl)-3,3- difluorocyclobutane-1-carboxamide |
| 496 | 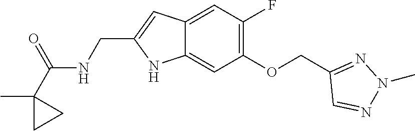 | N-((5-fluoro-6-((2-methyl-2H-1,2,3- triazol-4-yl)methoxy)-1H-indol-2- yl)methyl)-1-methylcyclopropane-1- carboxamide |
| 497 | 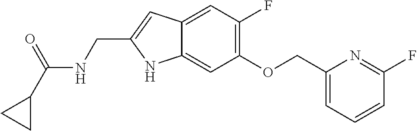 [SMILES: O=C(NCc1cc2cc(F)c(OCc3cccc(F)n3)cc2[nH]1)C1CC1] | N-((5-fluoro-6-((6-fluoropyridin-2- yl)methoxy)-1H-indol-2- yl)methyl)cyclopropanecarboxamide |
| 498 | 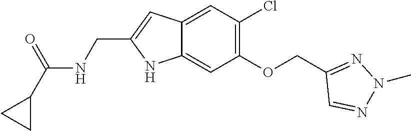 [SMILES: Cn1ncc(COc2cc3[nH]c(CNC(=O)C4CC4)cc3cc2Cl)n1] | N-((5-chloro-6-((2-methyl-2H-1,2,3- triazol-4-yl)methoxy)-1H-indol-2- yl)methyl)cyclopropanecarboxamide |
| 499 | 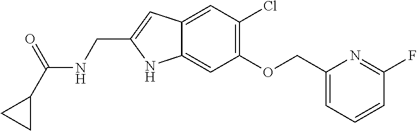 [SMILES: O=C(NCc1cc2cc(Cl)c(OCc3cccc(F)n3)cc2[nH]1)C1CC1] | N-((5-chloro-6-((6-fluoropyridin-2- yl)methoxy)-1H-indol-2- yl)methyl)cyclopropanecarboxamide |
| 500 | 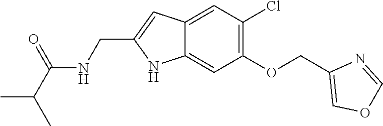 [SMILES: CC(C)C(=O)NCc1cc2cc(Cl)c(OCc3cocn3)cc2[nH]1] | N-((5-chloro-6-(oxazol-4-ylmethoxy)- 1H-indol-2-yl)methyl)isobutyramide |
| 501 | 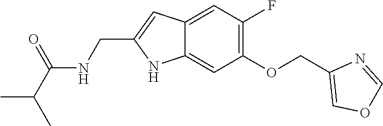 [SMILES: CC(C)C(=O)NCc1cc2cc(F)c(OCc3cocn3)cc2[nH]1] | N-((5-fluoro-6-(oxazol-4-ylmethoxy)- 1H-indol-2-yl)methyl)isobutyramide |
| 502 | 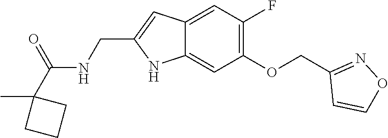 [SMILES: CC1(C(=O)NCc2cc3cc(F)c(OCc4ccon4)cc3[nH]2)CCC1] | N-((5-fluoro-6-(isoxazol-3-ylmethoxy)- 1H-indol-2-yl)methyl)-1- methylcyclobutane-1-carboxamide |
| 503 | 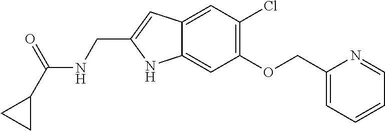 [SMILES: O=C(NCc1cc2cc(Cl)c(OCc3ccccn3)cc2[nH]1)C1CC1] | N-((5-chloro-6-(pyridin-2-ylmethoxy)- 1H-indol-2- yl)methyl)cyclopropanecarboxamide |
| 504 | 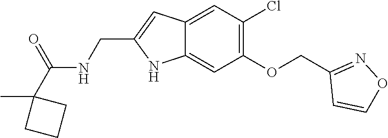 [SMILES: CC1(C(=O)NCc2cc3cc(Cl)c(OCc4ccon4)cc3[nH]2)CCC1] | N-((5-chloro-6-(isoxazol-3-ylmethoxy)- 1H-indol-2-yl)methyl)-1- methylcyclobutane-1-carboxamide |
| 505 | 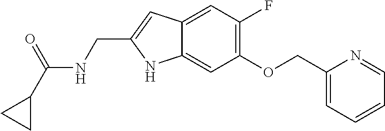 [SMILES: O=C(NCc1cc2cc(F)c(OCc3ccccn3)cc2[nH]1)C1CC1] | N-((5-fluoro-6-(pyridin-2-ylmethoxy)- 1H-indol-2- yl)methyl)cyclopropanecarboxamide |
| 506 | 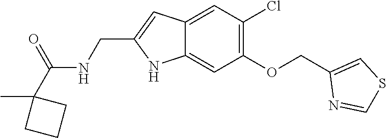 [SMILES: CC1(C(=O)NCc2cc3cc(Cl)c(OCc4cscn4)cc3[nH]2)CCC1] | N-((5-chloro-6-(thiazol-4-ylmethoxy)- 1H-indol-2-yl)methyl)-1- methylcyclobutane-1-carboxamide |
| 507 | 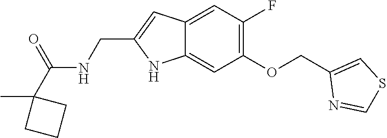 [SMILES: CC1(C(=O)NCc2cc3cc(F)c(OCc4cscn4)cc3[nH]2)CCC1] | N-((5-fluoro-6-(thiazol-4-ylmethoxy)- 1H-indol-2-yl)methyl)-1- methylcyclobutane-1-carboxamide |
| 508 | 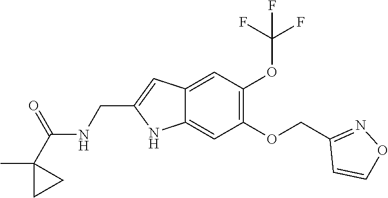 [SMILES: CC1(C(=O)NCc2cc3cc(OC(F)(F)F)c(OCc4ccon4)cc3[nH]2)CC1] | N-((6-(isoxazol-3-ylmethoxy)-5- (trifluoromethoxy)-1H-indol-2- yl)methyl)-1-methylcyclopropane-1- carboxamide |
| 509 | 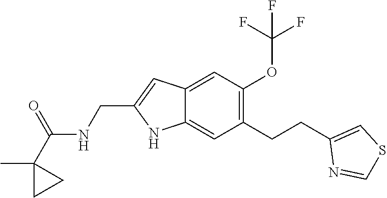 [SMILES: CC1(C(=O)NCc2cc3cc(OC(F)(F)F)c(CCc4cscn4)cc3[nH]2)CC1] | 1-methyl-N-((6-(2-(thiazol-4-yl)ethyl)- 5-(trifluoromethoxy)-1H-indol-2- yl)methyl)cyclopropane-1-carboxamide |
| 510 | 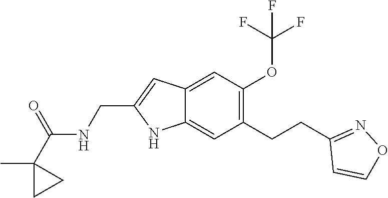 [SMILES: CC1(C(=O)NCc2cc3cc(OC(F)(F)F)c(CCc4ccon4)cc3[nH]2)CC1] | N-((6-(2-(isoxazol-3-yl)ethyl)-5- (trifluoromethoxy)-1H-indol-2- yl)methyl)-1-methylcyclopropane-1- carboxamide |
| 511 | 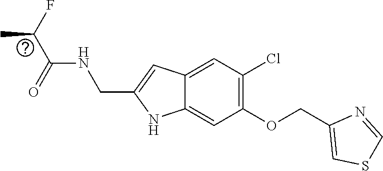 [SMILES: C[C@@H](F)C(=O)NCc1cc2cc(Cl)c(OCc3cscn3)cc2[nH]1] | (R)-N-((5-chloro-6-(thiazol-4- ylmethoxy)-1H-indol-2-yl)methyl)-2- fluoropropanamide and (S)-N-((5- chloro-6-(thiazol-4-ylmethoxy)-1H- indol-2-yl)methyl)-2- fluoropropanamide |
| 512 | 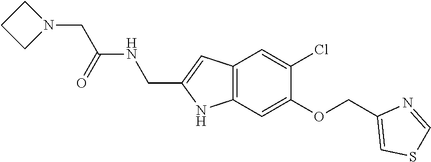 [SMILES: O=C(CN1CCC1)NCc1cc2cc(Cl)c(OCc3cscn3)cc2[nH]1] | 2-(azetidin-1-yl)-N-((5-chloro-6- (thiazol-4-ylmethoxy)-1H-indol-2- yl)methyl)acetamide |
| 513 | 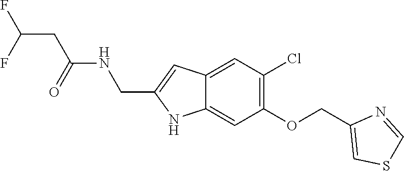 [SMILES: O=C(CC(F)F)NCc1cc2cc(Cl)c(OCc3cscn3)cc2[nH]1] | N-((5-chloro-6-(thiazol-4-ylmethoxy)- 1H-indol-2-yl)methyl)-3,3- difluoropropanamide |
| 514 | 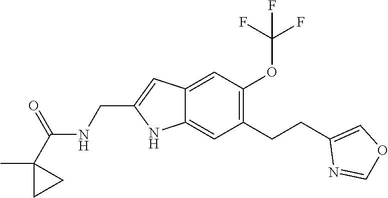 [SMILES: CC1(C(=O)NCc2cc3cc(OC(F)(F)F)c(CCc4cocn4)cc3[nH]2)CC1] | 1-methyl-N-((6-(2-(oxazol-4-yl)ethyl)- 5-(trifluoromethoxy)-1H-indol-2- yl)methyl)cyclopropane-1-carboxamide |
| 515 | 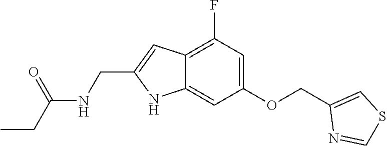 [SMILES: CCC(=O)NCc1cc2c(F)cc(OCc3cscn3)cc2[nH]1] | N-((4-fluoro-6-(thiazol-4-ylmethoxy)- 1H-indol-2-yl)methyl)propionamide |
| 516 | 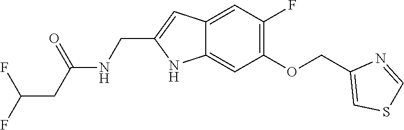 [SMILES: O=C(CC(F)F)NCc1cc2cc(F)c(OCc3cscn3)cc2[nH]1] | 3,3-difluoro-N-((5-fluoro-6-(thiazol-4- ylmethoxy)-1H-indol-2- yl)methyl)propanamide |
| 517 | 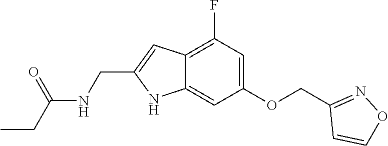 [SMILES: CCC(=O)NCc1cc2c(F)cc(OCc3ccon3)cc2[nH]1] | N-((4-fluoro-6-(isoxazol-3-ylmethoxy)- 1H-indol-2-yl)methyl)propionamide |
| 518 | 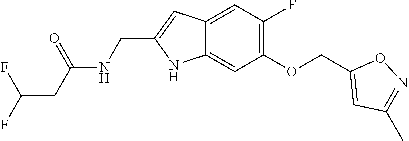 [SMILES: Cc1cc(COc2cc3[nH]c(CNC(=O)CC(F)F)cc3cc2F)on1] | 3,3-difluoro-N-((5-fluoro-6-((3- methylisoxazol-5-yl)methoxy)-1H- indol-2-yl)methyl)propanamide |
| 519 | 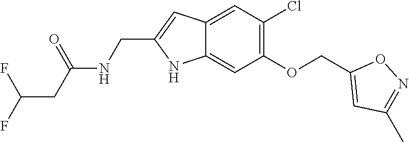 [SMILES: Cc1cc(COc2cc3[nH]c(CNC(=O)CC(F)F)cc3cc2Cl)on1] | N-((5-chloro-6-((3-methylisoxazol-5- yl)methoxy)-1H-indol-2-yl)methyl)- 3,3-difluoropropanamide |
| 520 | 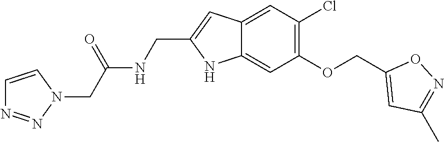 [SMILES: Cc1cc(COc2cc3[nH]c(CNC(=O)Cn4ccnn4)cc3cc2Cl)on1] | N-((5-chloro-6-((3-methylisoxazol-5- yl)methoxy)-1H-indol-2-yl)methyl)-2- (1H-1,2,3-triazol-1-yl)acetamide |
| 521 | 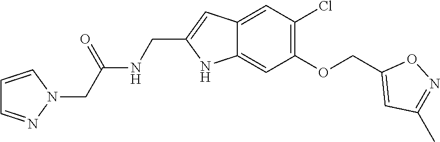 [SMILES: Cc1cc(COc2cc3[nH]c(CNC(=O)Cn4cccn4)cc3cc2Cl)on1] | N-((5-chloro-6-((3-methylisoxazol-5- yl)methoxy)-1H-indol-2-yl)methyl)-2- (1H-pyrazol-1-yl)acetamide |
| 522 | 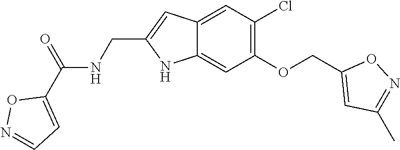 [SMILES: Cc1cc(COc2cc3[nH]c(CNC(=O)c4ccno4)cc3cc2Cl)on1] | N-((5-chloro-6-((3-methylisoxazol-5- yl)methoxy)-1H-indol-2- yl)methyl)isoxazole-5-carboxamide |
| 523 | 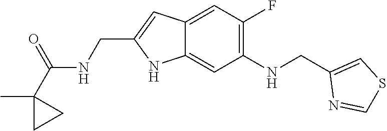 [SMILES: CC1(C(=O)NCc2cc3cc(F)c(NCc4cscn4)cc3[nH]2)CC1] | N-((5-fluoro-6-((thiazol-4- ylmethyl)amino)-1H-indol-2- yl)methyl)-1-methylcyclopropane-1- carboxamide |
| 524 | 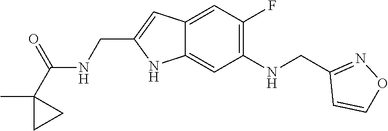 [SMILES: CC1(C(=O)NCc2cc3cc(F)c(NCc4ccon4)cc3[nH]2)CC1] | N-((5-fluoro-6-((isoxazol-3- ylmethyl)amino)-1H-indol-2- yl)methyl)-1-methylcyclopropane-1- carboxamide |
| 525 | 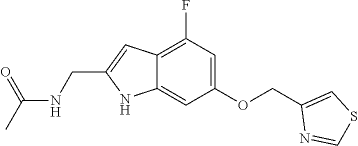 [SMILES: CC(=O)NCc1cc2c(F)cc(OCc3cscn3)cc2[nH]1] | N-((4-fluoro-6-(thiazol-4-ylmethoxy)- 1H-indol-2-yl)methyl)acetamide |
| 526 | 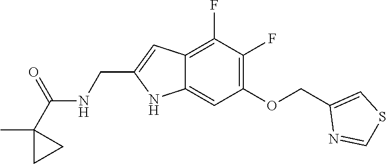 [SMILES: CC1(C(=O)NCc2cc3c(F)c(F)c(OCc4cscn4)cc3[nH]2)CC1] | N-((4,5-difluoro-6-(thiazol-4- ylmethoxy)-1H-indol-2-yl)methyl)-1- methylcyclopropane-1-carboxamide |
| 527 | 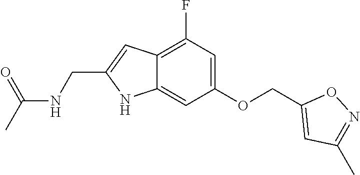 [SMILES: CC(=O)NCc1cc2c(F)cc(OCc3cc(C)no3)cc2[nH]1] | N-((4-fluoro-6-((3-methylisoxazol-5- yl)methoxy)-1H-indol-2- yl)methyl)acetamide |
| 528 | 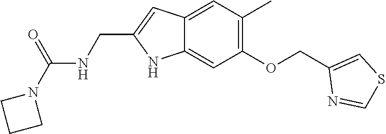 [SMILES: Cc1cc2cc(CNC(=O)N3CCC3)[nH]c2cc1OCc1cscn1] | N-((5-methyl-6-(thiazol-4-ylmethoxy)- 1H-indol-2-yl)methyl)azetidine-1- carboxamide |
| 529 | 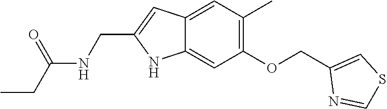 [SMILES: CCC(=O)NCc1cc2cc(C)c(OCc3cscn3)cc2[nH]1] | N-((5-methyl-6-(thiazol-4-ylmethoxy)- 1H-indol-2-yl)methyl)propionamide |
| 530 | 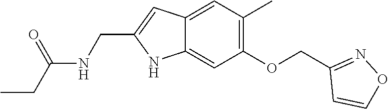 [SMILES: CCC(=O)NCc1cc2cc(C)c(OCc3ccon3)cc2[nH]1] | N-((6-(isoxazol-3-ylmethoxy)-5- methyl-1H-indol-2- yl)methyl)propionamide |
| 531 | 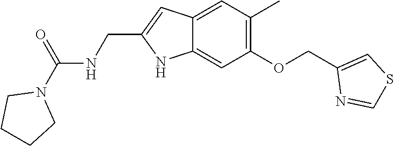 [SMILES: Cc1cc2cc(CNC(=O)N3CCCC3)[nH]c2cc1OCc1cscn1] | N-((5-methyl-6-(thiazol-4-ylmethoxy)- 1H-indol-2-yl)methyl)pyrrolidine-1- carboxamide |
| 532 | 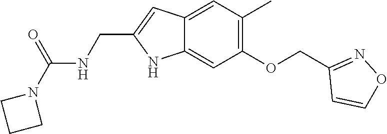 [SMILES: Cc1cc2cc(CNC(=O)N3CCC3)[nH]c2cc1OCc1ccon1] | N-((6-(isoxazol-3-ylmethoxy)-5- methyl-1H-indol-2-yl)methyl)azetidine- 1-carboxamide |
| 533 | 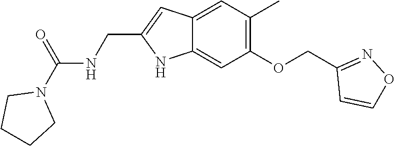 [SMILES: Cc1cc2cc(CNC(=O)N3CCCC3)[nH]c2cc1OCc1ccon1] | N-((6-(isoxazol-3-ylmethoxy)-5- methyl-1H-indol-2- yl)methyl)pyrrolidine-1-carboxamide |
| 534 | 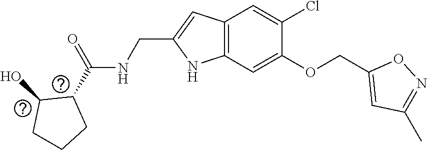 [SMILES: Cc1cc(COc2cc3[nH]c(CNC(=O)[C@@H]4CCC[C@H]4O)cc3cc2Cl)on1] | (1R,2R)-N-((5-chloro-6-((3- methylisoxazol-5-yl)methoxy)-1H- indol-2-yl)methyl)-2- hydroxycyclopentane-1-carboxamide and (1S,2S)-N-((5-chloro-6-((3- methylisoxazol-5-yl)methoxy)-1H- indol-2-yl)methyl)-2- hydroxycyclopentane-1-carboxamide |
| 535 | 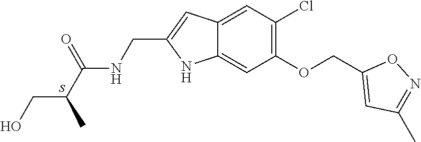 | (S)-N-((5-chloro-6-((3-methylisoxazol- 5-yl)methoxy)-1H-indol-2-yl)methyl)- 3-hydroxy-2-methylpropanamide |
| 536 | 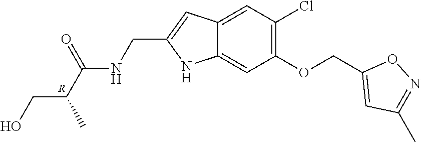 | (R)-N-((5-chloro-6-((3-methylisoxazol- 5-yl)methoxy)-1H-indol-2-yl)methyl)- 3-hydroxy-2-methylpropanamide |
| 537 | 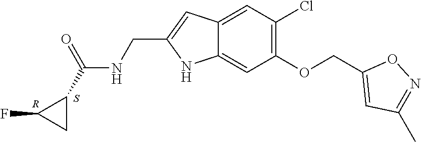 | (1S,2R)-N-((5-chloro-6-((3- methylisoxazol-5-yl)methoxy)-1H- indol-2-yl)methyl)-2- fluorocyclopropane-1-carboxamide |
| 538 | 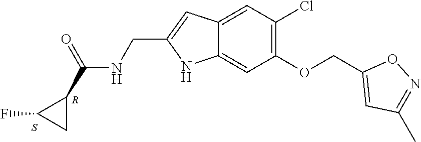 | (1R,2S)-N-((5-chloro-6-((3- methylisoxazol-5-yl)methoxy)-1H- indol-2-yl)methyl)-2- fluorocyclopropane-1-carboxamide |
| 539 | 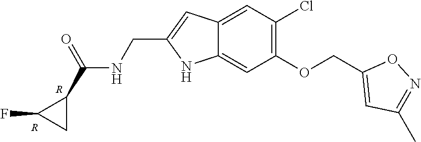 | (1R,2R)-N-((5-chloro-6-((3- methylisoxazol-5-yl)methoxy)-1H- indol-2-yl)methyl)-2- fluorocyclopropane-1-carboxamide |
| 540 | 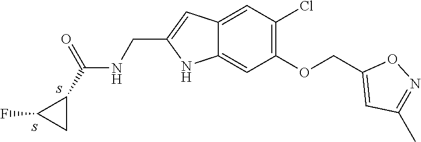 | (1S,2S)-N-((5-chloro-6-((3- methylisoxazol-5-yl)methoxy)-1H- indol-2-yl)methyl)-2- fluorocyclopropane-1-carboxamide |
| 541 | 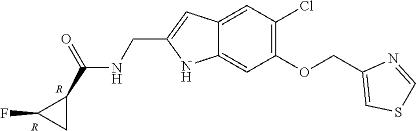 | (1R,2R)-N-((5-chloro-6-(thiazol-4- ylmethoxy)-1H-indol-2-yl)methyl)-2- fluorocyclopropane-1-carboxamide |
| 542 | 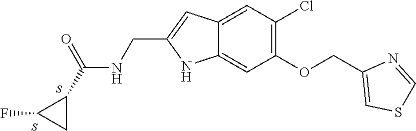 | (1S,2S)-N-((5-chloro-6-(thiazol-4- ylmethoxy)-1H-indol-2-yl)methyl)-2- fluorocyclopropane-1-carboxamide |
| 543 | 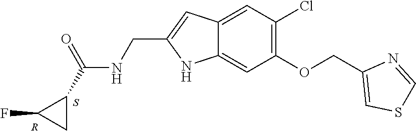 | (1S,2R)-N-((5-chloro-6-(thiazol-4- ylmethoxy)-1H-indol-2-yl)methyl)-2- fluorocyclopropane-1-carboxamide |
| 544 | 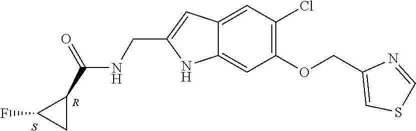 | (1R,2S)-N-((5-chloro-6-(thiazol-4- ylmethoxy)-1H-indol-2-yl)methyl)-2- fluorocyclopropane-1-carboxamide |
| 545 | 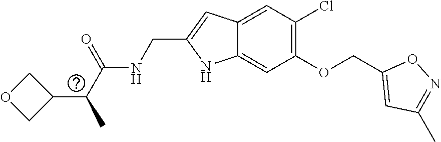 [SMILES: Cc1cc(COc2cc3[nH]c(CNC(=O)[C@@H](C)C4COC4)cc3cc2Cl)on1] | (S)-N-((5-chloro-6-((3-methylisoxazol- 5-yl)methoxy)-1H-indol-2-yl)methyl)- 2-(oxetan-3-yl)propenamide and (R)-N- ((5-chloro-6-((3-methylisoxazol-5- yl)methoxy)-1H-indol-2-yl)methyl)-2- (oxetan-3-yl)propanamide |
| 546 | 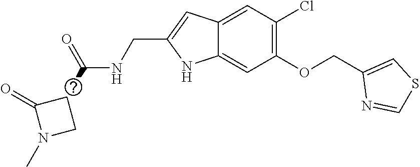 [SMILES: CN1C[C@H](C(=O)NCc2cc3cc(Cl)c(OCc4cscn4)cc3[nH]2)C1=O] | (R)-N-((5-chloro-6-(thiazol-4- ylmethoxy)-1H-indol-2-yl)methyl)-1- methyl-2-oxoazetidine-3-carboxamide and (S)-N-((5-chloro-6-(thiazol-4- ylmethoxy)-1H-indol-2-yl)methyl)-1- methyl-2-oxoazetidine-3-carboxamide |
| 547 | 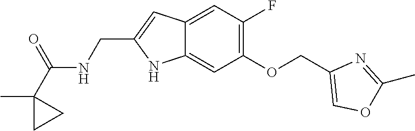 | N-((5-fluoro-6-((2-methyloxazol-4- yl)methoxy)-1H-indol-2-yl)methyl)-1- methylcyclopropane-1-carboxamide |
| 548 | 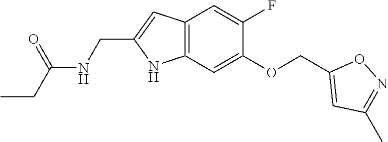 [SMILES: CCC(=O)NCc1cc2cc(F)c(OCc3cc(C)no3)cc2[nH]1] | N-((5-fluoro-6-((3-methylisoxazol-5- yl)methoxy)-1H-indol-2- yl)methyl)propionamide |
| 549 | 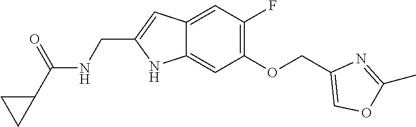 [SMILES: Cc1nc(COc2cc3[nH]c(CNC(=O)C4CC4)cc3cc2F)co1] | N-((5-fluoro-6-((2-methyloxazol-4- yl)methoxy)-1H-indol-2- yl)methyl)cyclopropanecarboxamide |
| 550 | 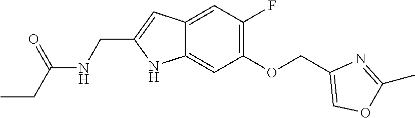 [SMILES: CCC(=O)NCc1cc2cc(F)c(OCc3coc(C)n3)cc2[nH]1] | N-((5-fluoro-6-((2-methyloxazol-4- yl)methoxy)-1H-indol-2- yl)methyl)propionamide |
| 551 | 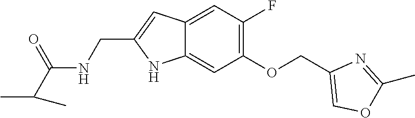 [SMILES: Cc1nc(COc2cc3[nH]c(CNC(=O)C(C)C)cc3cc2F)co1] | N-((5-fluoro-6-((2-methyloxazol-4- yl)methoxy)-1H-indol-2- yl)methyl)isobutyramide |
| 552 | 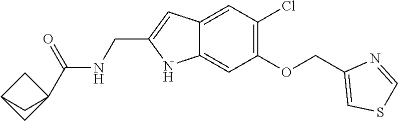 [SMILES: O=C(NCc1cc2cc(Cl)c(OCc3cscn3)cc2[nH]1)C12CC(C1)C2] | N-((5-chloro-6-(thiazol-4-ylmethoxy)- 1H-indol-2- yl)methyl)bicyclo[1.1.1]pentane-1- carboxamide |
| 553 | 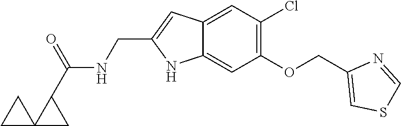 [SMILES: O=C(NCc1cc2cc(Cl)c(OCc3cscn3)cc2[nH]1)C1CC12CC2] | N-((5-chloro-6-(thiazol-4-ylmethoxy)- 1H-indol-2- yl)methyl)spiro[2.2]pentane-1- carboxamide |
| 554 | 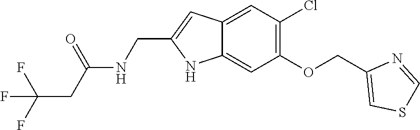 [SMILES: O=C(CC(F)(F)F)NCc1cc2cc(Cl)c(OCc3cscn3)cc2[nH]1] | N-((5-chloro-6-(thiazol-4-ylmethoxy)- 1H-indol-2-yl)methyl)-3,3,3- trifluoropropanamide |
| 555 | 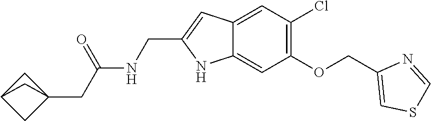 [SMILES: O=C(CC12CC(C1)C2)NCc1cc2cc(Cl)c(OCc3cscn3)cc2[nH]1] | 2-(bicyclo[1.1.1]pentan-1-yl)-N-((5- chloro-6-(thiazol-4-ylmethoxy)-1H- indol-2-yl)methyl)acetamide |
| 556 | 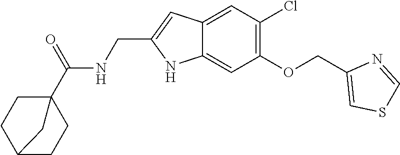 [SMILES: O=C(NCc1cc2cc(Cl)c(OCc3cscn3)cc2[nH]1)C12CCC(CC1)C2] | N-((5-chloro-6-(thiazol-4-ylmethoxy)- 1H-indol-2- yl)methyl)bicyclo[2.2.1]heptane-1- carboxamide |
| 557 | 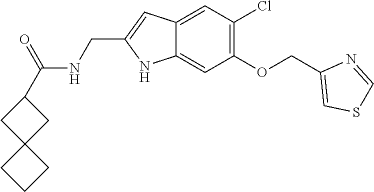 [SMILES: O=C(NCc1cc2cc(Cl)c(OCc3cscn3)cc2[nH]1)C1CC2(CCC2)C1] | N-((5-chloro-6-(thiazol-4-ylmethoxy)- 1H-indol-2- yl)methyl)spiro[3.3]heptane-2- carboxamide |
| 558 | 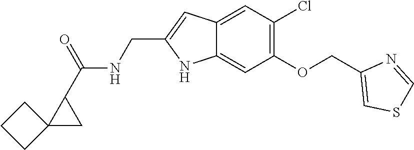 [SMILES: O=C(NCc1cc2cc(Cl)c(OCc3cscn3)cc2[nH]1)C1CC12CCC2] | N-((5-chloro-6-(thiazol-4-ylmethoxy)- 1H-indol-2- yl)methyl)spiro[2.3]hexane-1- carboxamide |
| 559 | 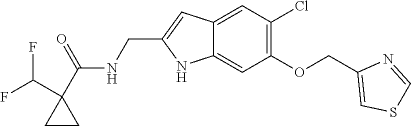 [SMILES: O=C(NCc1cc2cc(Cl)c(OCc3cscn3)cc2[nH]1)C1(C(F)F)CC1] | N-((5-chloro-6-(thiazol-4-ylmethoxy)- 1H-indol-2-yl)methyl)-1- (difluoromethyl)cyclopropane-1- carboxamide |
| 560 | 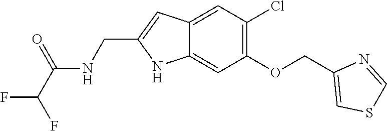 [SMILES: O=C(NCc1cc2cc(Cl)c(OCc3cscn3)cc2[nH]1)C(F)F] | N-((5-chloro-6-(thiazol-4-ylmethoxy)- 1H-indol-2-yl)methyl)-2,2- difluoroacetamide |
| 561 | 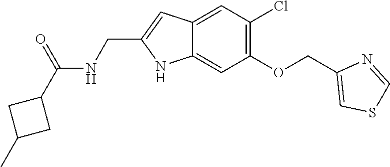 [SMILES: CC1CC(C(=O)NCc2cc3cc(Cl)c(OCc4cscn4)cc3[nH]2)C1] | N-((5-chloro-6-(thiazol-4-ylmethoxy)- 1H-indol-2-yl)methyl)-3- methylcyclobutane-1-carboxamide |
| 562 | 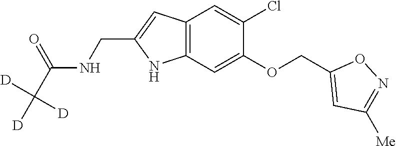 [SMILES: [2H]C([2H])([2H])C(=O)NCc1cc2cc(Cl)c(OCc3cc(C)no3)cc2[nH]1] | N-((5-chloro-6-((3-methylisoxazol-5- yl)methoxy)-1H-indol-2- yl)methyl)acetamide-2,2,2-d3 |
| 563 | 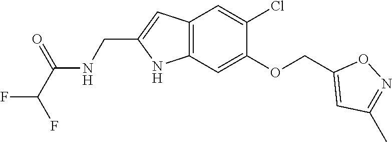 [SMILES: Cc1cc(COc2cc3[nH]c(CNC(=O)C(F)F)cc3cc2Cl)on1] | N-((5-chloro-6-((3-methylisoxazol-5- yl)methoxy)-1H-indol-2-yl)methyl)- 2,2-difluoroacetamide |
| 564 | 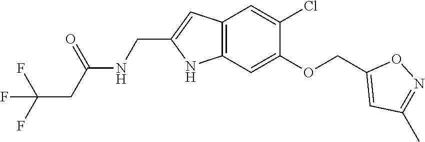 [SMILES: Cc1cc(COc2cc3[nH]c(CNC(=O)CC(F)(F)F)cc3cc2Cl)on1] | N-((5-chloro-6-((3-methylisoxazol-5- yl)methoxy)-1H-indol-2-yl)methyl)- 3,3,3-trifluoropropanamide |
| 565 | 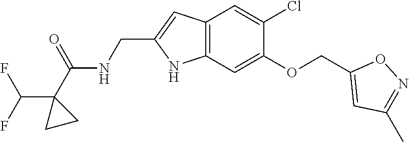 [SMILES: Cc1cc(COc2cc3[nH]c(CNC(=O)C4(C(F)F)CC4)cc3cc2Cl)on1] | N-((5-chloro-6-((3-methylisoxazol-5- yl)methoxy)-1H-indol-2-yl)methyl)-1- (difluoromethyl)cyclopropane-1- carboxamide |
| 566 | 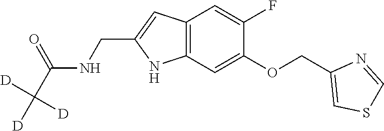 [SMILES: [2H]C([2H])([2H])C(=O)NCc1cc2cc(F)c(OCc3cscn3)cc2[nH]1] | N-((5-fluoro-6-(thiazol-4-ylmethoxy)- 1H-indol-2-yl)methyl)acetamide-2,2,2- d3 |
| 567 | 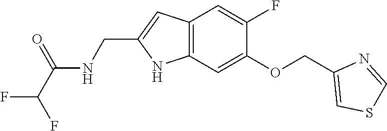 [SMILES: O=C(NCc1cc2cc(F)c(OCc3cscn3)cc2[nH]1)C(F)F] | 2,2-difluoro-N-((5-fluoro-6-(thiazol-4- ylmethoxy)-1H-indol-2- yl)methyl)acetamide |
| 568 | 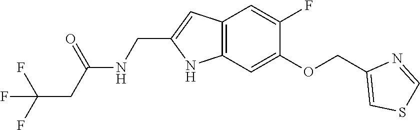 [SMILES: O=C(CC(F)(F)F)NCc1cc2cc(F)c(OCc3cscn3)cc2[nH]1] | 3,3,3-trifluoro-N-((5-fluoro-6-(thiazol- 4-ylmethoxy)-1H-indol-2- yl)methyl)propanamide |
| 569 | 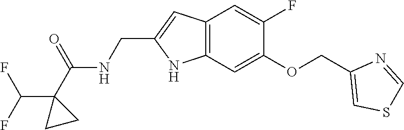 [SMILES: O=C(NCc1cc2cc(F)c(OCc3cscn3)cc2[nH]1)C1(C(F)F)CC1] | 1-(difluoromethyl)-N-((5-fluoro-6- (thiazol-4-ylmethoxy)-1H-indol-2- yl)methyl)cyclopropane-1-carboxamide |
| 570 | 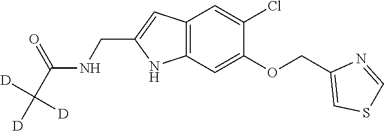 [SMILES: [2H]C([2H])([2H])C(=O)NCc1cc2cc(Cl)c(OCc3cscn3)cc2[nH]1] | N-((5-chloro-6-(thiazol-4-ylmethoxy)- 1H-indol-2-yl)methyl)acetamide-2,2,2- d3 |
| 571 | 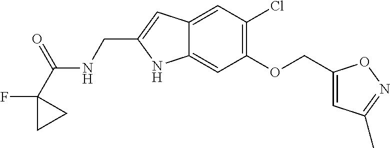 [SMILES: Cc1cc(COc2cc3[nH]c(CNC(=O)C4(F)CC4)cc3cc2Cl)on1] | N-((5-chloro-6-((3-methylisoxazol-5- yl)methoxy)-1H-indol-2-yl)methyl)-1- fluorocyclopropane-1-carboxamide |
| 572 | 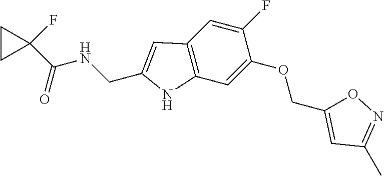 [SMILES: Cc1cc(COc2cc3[nH]c(CNC(=O)C4(F)CC4)cc3cc2F)on1] | 1-fluoro-N-((5-fluoro-6-((3- methylisoxazol-5-yl)methoxy)-1H- indol-2-yl)methyl)cyclopropane-1- carboxamide |
| 573 | 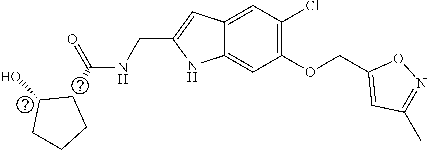 [SMILES: Cc1cc(COc2cc3[nH]c(CNC(=O)[C@@H]4CCC[C@@H]4O)cc3cc2Cl)on1] | (1R,2S)-N-((5-chloro-6-((3- methylisoxazol-5-yl)methoxy)-1H- indol-2-yl)methyl)-2- hydroxycyclopentane-1-carboxamide and (1S,2R)-N-((5-chloro-6-((3- methylisoxazol-5-yl)methoxy)-1H- indol-2-yl)methyl)-2- hydroxycyclopentane-1-carboxamide |
| 574 | 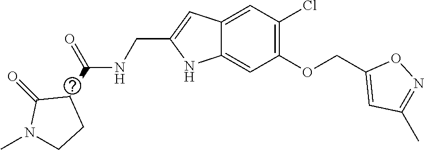 | (R)-N-((5-chloro-6-((3-methylisoxazol- 5-yl)methoxy)-1H-indol-2-yl)methyl)- 1-methyl-2-oxopyrrolidine-3- carboxamide and (S)-N-((5-chloro-6- ((3-methylisoxazol-5-yl)methoxy)-1H- indol-2-yl)methyl)-1-methyl-2- oxopyrrolidine-3-carboxamide |
| 575 | 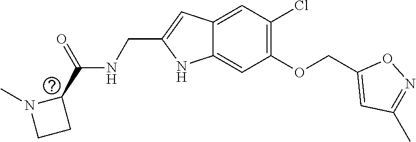 | (R)-N-((5-chloro-6-((3-methylisoxazol- 5-yl)methoxy)-1H-indol-2-yl)methyl)- 1-methylazetidine-2-carboxamide |
| 576 | 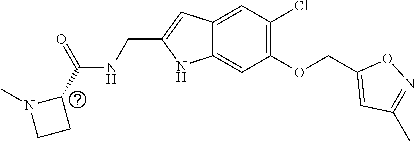 | (S)-N-((5-chloro-6-((3-methylisoxazol- 5-yl)methoxy)-1H-indol-2-yl)methyl)- 1-methylazetidine-2-carboxamide |
| 577 | 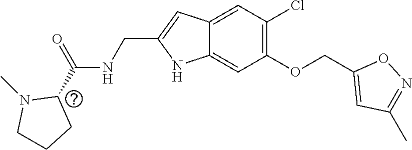 | (S)-N-((5-chloro-6-((3-methylisoxazol- 5-yl)methoxy)-1H-indol-2-yl)methyl)- 1-methylpyrrolidine-2-carboxamide |
| 578 | 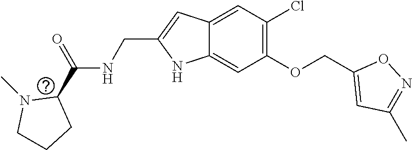 | (R)-N-((5-chloro-6-((3-methylisoxazol- 5-yl)methoxy)-1H-indol-2-yl)methyl)- 1-methylpyrrolidine-2-carboxamide |
| 579 | 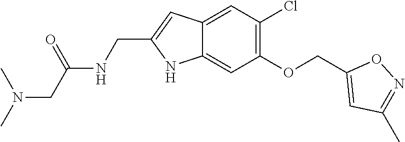 | N-((5-chloro-6-((3-methylisoxazol-5- yl)methoxy)-1H-indol-2-yl)methyl)-2- (dimethylamino)acetamide |
| 580 | 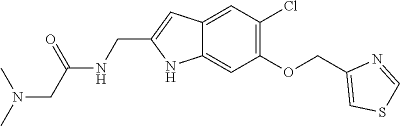 [SMILES: CN(C)CC(=O)NCc1cc2cc(Cl)c(OCc3cscn3)cc2[nH]1] | N-((5-chloro-6-(thiazol-4-ylmethoxy)- 1H-indol-2-yl)methyl)-2- (dimethylamino)acetamide |
| 581 | 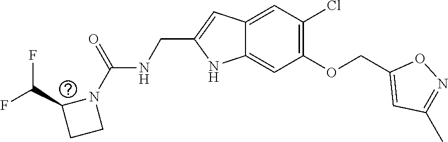 [SMILES: Cc1cc(COc2cc3[nH]c(CNC(=O)N4CC[C@H]4C(F)F)cc3cc2Cl)on1] | (S)-N-((5-chloro-6-((3-methylisoxazol- 5-yl)methoxy)-1H-indol-2-yl)methyl)- 2-(difluoromethyl)azetidine-1- carboxamide and (R)-N-((5-chloro-6- ((3-methylisoxazol-5-yl)methoxy)-1H- indol-2-yl)methyl)-2- (difluoromethyl)azetidine-1- carboxamide |
| 582 | 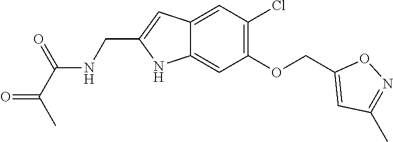 [SMILES: CC(=O)C(=O)NCc1cc2cc(Cl)c(OCc3cc(C)no3)cc2[nH]1] | N-((5-chloro-6-((3-methylisoxazol-5- yl)methoxy)-1H-indol-2-yl)methyl)-2- oxopropanamide |
| 583 | 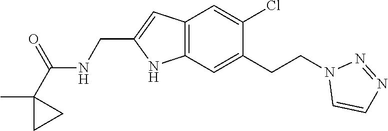 [SMILES: CC1(C(=O)NCc2cc3cc(Cl)c(CCn4ccnn4)cc3[nH]2)CC1] | N-((6-(2-(1H-1,2,3-triazol-1-yl)ethyl)- 5-chloro-1H-indol-2-yl)methyl)-1- methylcyclopropane-1-carboxamide |
| 584 | 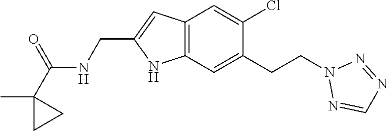 [SMILES: CC1(C(=O)NCc2cc3cc(Cl)c(CCn4ncnn4)cc3[nH]2)CC1] | N-((6-(2-(2H-tetrazol-2-yl)ethyl)-5- chloro-1H-indol-2-yl)methyl)-1- methylcyclopropane-1-carboxamide |
| 585 | 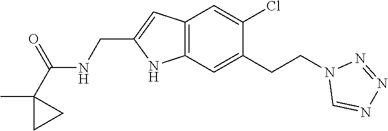 [SMILES: CC1(C(=O)NCc2cc3cc(Cl)c(CCn4cnnn4)cc3[nH]2)CC1] | N-((6-(2-(1H-tetrazol-1-yl)ethyl)-5- chloro-1H-indol-2-yl)methyl)-1- methylcyclopropane-1-carboxamide |
| 586 | 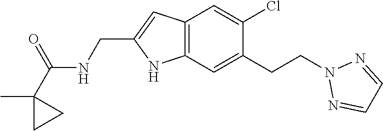 [SMILES: CC1(C(=O)NCc2cc3cc(Cl)c(CCn4nccn4)cc3[nH]2)CC1] | N-((6-(2-(2H-1,2,3-triazol-2-yl)ethyl)- 5-chloro-1H-indol-2-yl)methyl)-1- methylcyclopropane-1-carboxamide |
| 587 | 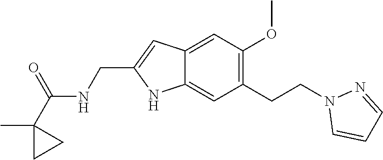 [SMILES: COc1cc2cc(CNC(=O)C3(C)CC3)[nH]c2cc1CCn1cccn1] | N-((6-(2-(1H-pyrazol-1-yl)ethyl)-5- methoxy-1H-indol-2-yl)methyl)-1- methylcyclopropane-1-carboxamide |
| 588 | 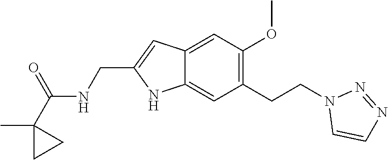 [SMILES: COc1cc2cc(CNC(=O)C3(C)CC3)[nH]c2cc1CCn1ccnn1] | N-((6-(2-(1H-1,2,3-triazol-1-yl)ethyl)- 5-methoxy-1H-indol-2-yl)methyl)-1- methylcyclopropane-1-carboxamide |
| 589 | 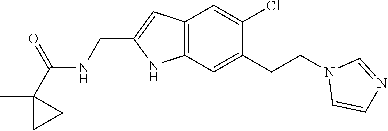 [SMILES: CC1(C(=O)NCc2cc3cc(Cl)c(CCn4ccnc4)cc3[nH]2)CC1] | N-((6-(2-(1H-imidazol-1-yl)ethyl)-5- chloro-1H-indol-2-yl)methyl)-1- methylcyclopropane-1-carboxamide |
| 590 | 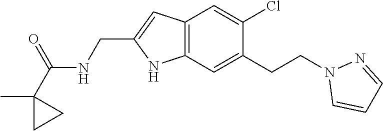 [SMILES: CC1(C(=O)NCc2cc3cc(Cl)c(CCn4cccn4)cc3[nH]2)CC1] | N-((6-(2-(1H-pyrazol-1-yl)ethyl)-5- chloro-1H-indol-2-yl)methyl)-1- methylcyclopropane-1-carboxamide |
| 591 | 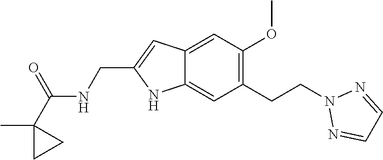 [SMILES: COc1cc2cc(CNC(=O)C3(C)CC3)[nH]c2cc1CCn1nccn1] | N-((6-(2-(2H-1,2,3-triazol-2-yl)ethyl)- 5-methoxy-1H-indol-2-yl)methyl)-1- methylcyclopropane-1-carboxamide |
| 592 | 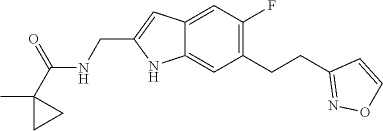 [SMILES: CC1(C(=O)NCc2cc3cc(F)c(CCc4ccon4)cc3[nH]2)CC1] | N-((5-fluoro-6-(2-(isoxazol-3-yl)ethyl)- 1H-indol-2-yl)methyl)-1- methylcyclopropane-1-carboxamide |
| 593 | 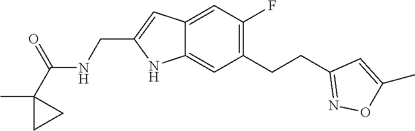 | N-((5-fluoro-6-(2-(5-methylisoxazol-3- yl)ethyl)-1H-indol-2-yl)methyl)-1- methylcyclopropane-1-carboxamide |
| 594 | 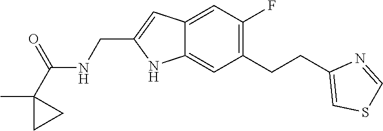 [SMILES: CC1(C(=O)NCc2cc3cc(F)c(CCc4cscn4)cc3[nH]2)CC1] | N-((5-fluoro-6-(2-(thiazol-4-yl)ethyl)- 1H-indol-2-yl)methyl)-1- methylcyclopropane-1-carboxamide |
| 595 | 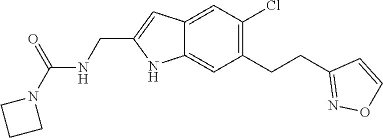 [SMILES: O=C(NCc1cc2cc(Cl)c(CCc3ccon3)cc2[nH]1)N1CCC1] | N-((5-chloro-6-(2-(isoxazol-3-yl)ethyl)- 1H-indol-2-yl)methyl)azetidine-1- carboxamide |
| 596 | 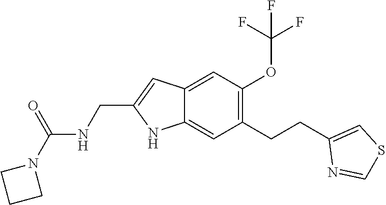 [SMILES: O=C(NCc1cc2cc(OC(F)(F)F)c(CCc3cscn3)cc2[nH]1)N1CCC1] | N-((6-(2-(thiazol-4-yl)ethyl)-5- (trifluoromethoxy)-1H-indol-2- yl)methyl)azetidine-1-carboxamide |
| 597 | 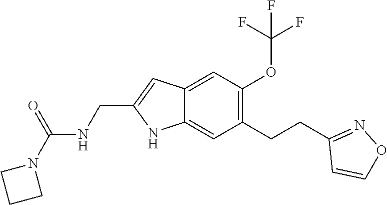 [SMILES: O=C(NCc1cc2cc(OC(F)(F)F)c(CCc3ccon3)cc2[nH]1)N1CCC1] | N-((6-(2-(isoxazol-3-yl)ethyl)-5- (trifluoromethoxy)-1H-indol-2- yl)methyl)azetidine-1-carboxamide |
| 598 | 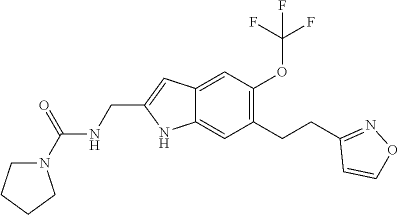 [SMILES: O=C(NCc1cc2cc(OC(F)(F)F)c(CCc3ccon3)cc2[nH]1)N1CCCC1] | N-((6-(2-(isoxazol-3-yl)ethyl)-5- (trifluoromethoxy)-1H-indol-2- yl)methyl)pyrrolidine-1-carboxamide |
| 599 | 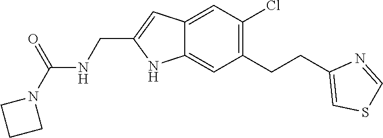 [SMILES: O=C(NCc1cc2cc(Cl)c(CCc3cscn3)cc2[nH]1)N1CCC1] | N-((5-chloro-6-(2-(thiazol-4-yl)ethyl)- 1H-indol-2-yl)methyl)azetidine-1- carboxamide |
| 600 | 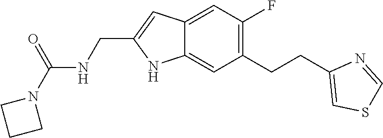 [SMILES: O=C(NCc1cc2cc(F)c(CCc3cscn3)cc2[nH]1)N1CCC1] | N-((5-fluoro-6-(2-(thiazol-4-yl)ethyl)- 1H-indol-2-yl)methyl)azetidine-1- carboxamide |
| 601 | 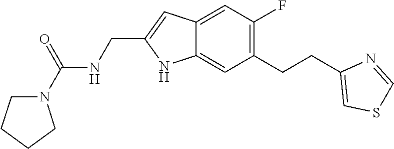 [SMILES: O=C(NCc1cc2cc(F)c(CCc3cscn3)cc2[nH]1)N1CCCC1] | N-((5-fluoro-6-(2-(thiazol-4-yl)ethyl)- 1H-indol-2-yl)methyl)pyrrolidine-1- carboxamide |
| 602 | 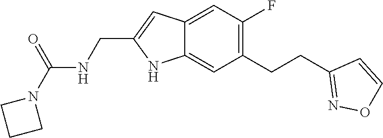 [SMILES: O=C(NCc1cc2cc(F)c(CCc3ccon3)cc2[nH]1)N1CCC1] | N-((5-fluoro-6-(2-(isoxazol-3-yl)ethyl)- 1H-indol-2-yl)methyl)azetidine-1- carboxamide |
| 603 | 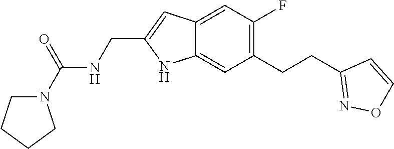 [SMILES: O=C(NCc1cc2cc(F)c(CCc3ccon3)cc2[nH]1)N1CCCC1] | N-((5-fluoro-6-(2-(isoxazol-3-yl)ethyl)- 1H-indol-2-yl)methyl)pyrrolidine-1- carboxamide |
| 604 | 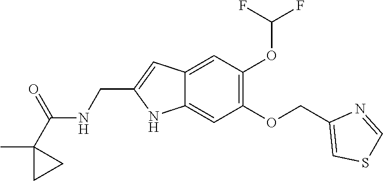 [SMILES: CC1(C(=O)NCc2cc3cc(OC(F)F)c(OCc4cscn4)cc3[nH]2)CC1] | N-((5-(difluoromethoxy)-6-(thiazol-4- ylmethoxy)-1H-indol-2-yl)methyl)-1- methylcyclopropane-1-carboxamide |
| 605 | 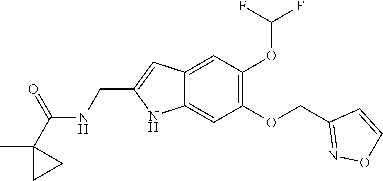 [SMILES: CC1(C(=O)NCc2cc3cc(OC(F)F)c(OCc4ccon4)cc3[nH]2)CC1] | N-((5-(difluoromethoxy)-6-(isoxazol-3- ylmethoxy)-1H-indol-2-yl)methyl)-1- methylcyclopropane-1-carboxamide |
| 606 | 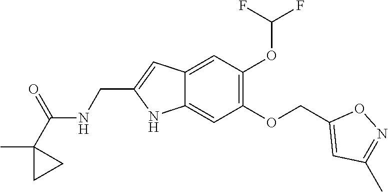 | N-((5-(difluoromethoxy)-6-((3- methylisoxazol-5-yl)methoxy)-1H- indol-2-yl)methyl)-1- methylcyclopropane-1-carboxamide |
| 607 | 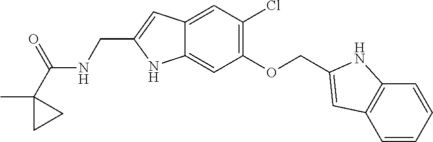 [SMILES: CC1(C(=O)NCc2cc3cc(Cl)c(OCc4cc5ccccc5[nH]4)cc3[nH]2)CC1] | N-((6-((1H-indol-2-yl)methoxy)-5- chloro-1H-indol-2-yl)methyl)-1- methylcyclopropane-1-carboxamide |
| 608 | 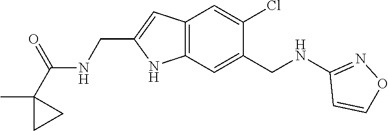 [SMILES: CC1(C(=O)NCc2cc3cc(Cl)c(CNc4ccon4)cc3[nH]2)CC1] | N-((5-chloro-6-((isoxazol-3- ylamino)methyl)-1H-indol-2- yl)methyl)-1-methylcyclopropane-1- carboxamide |
| 609 | 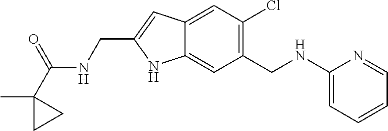 [SMILES: CC1(C(=O)NCc2cc3cc(Cl)c(CNc4ccccn4)cc3[nH]2)CC1] | N-((5-chloro-6-((pyridin-2- ylamino)methyl)-1H-indol-2- yl)methyl)-1-methylcyclopropane-1- carboxamide |
| 610 | 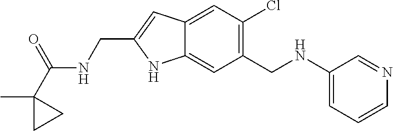 [SMILES: CC1(C(=O)NCc2cc3cc(Cl)c(CNc4cccnc4)cc3[nH]2)CC1] | N-((5-chloro-6-((pyridin-3- ylamino)methyl)-1H-indol-2- yl)methyl)-1-methylcyclopropane-1- carboxamide |
| 611 | 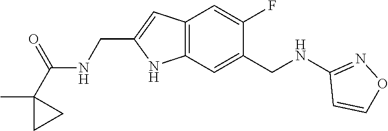 [SMILES: CC1(C(=O)NCc2cc3cc(F)c(CNc4ccon4)cc3[nH]2)CC1] | N-((5-fluoro-6-((isoxazol-3- ylamino)methyl)-1H-indol-2- yl)methyl)-1-methylcyclopropane-1- carboxamide |
| 612 | 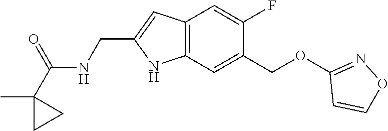 [SMILES: CC1(C(=O)NCc2cc3cc(F)c(COc4ccon4)cc3[nH]2)CC1] | N-((5-fluoro-6-((isoxazol-3- yloxy)methyl)-1H-indol-2-yl)methyl)- 1-methylcyclopropane-1-carboxamide |
| 613 | 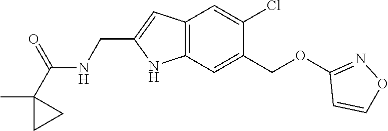 [SMILES: CC1(C(=O)NCc2cc3cc(Cl)c(COc4ccon4)cc3[nH]2)CC1] | N-((5-chloro-6-((isoxazol-3- yloxy)methyl)-1H-indol-2-yl)methyl)- 1-methylcyclopropane-1-carboxamide |
| 614 | 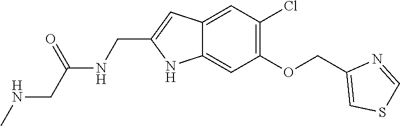 [SMILES: CNCC(=O)NCc1cc2cc(Cl)c(OCc3cscn3)cc2[nH]1] | N-((5-chloro-6-(thiazol-4-ylmethoxy)- 1H-indol-2-yl)methyl)-2- (methylamino)acetamide |
| 615 | 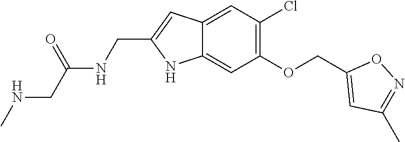 | N-((5-chloro-6-((3-methylisoxazol-5- yl)methoxy)-1H-indol-2-yl)methyl)-2- (methylamino)acetamide |
| 616 | 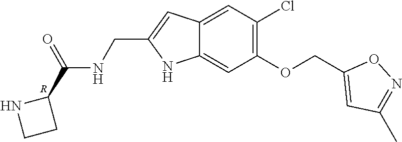 | (R)-N-((5-chloro-6-((3-methylisoxazol- 5-yl)methoxy)-1H-indol-2- yl)methyl)azetidine-2-carboxamide |
| 617 | 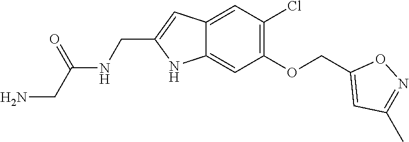 [SMILES: Cc1cc(COc2cc3[nH]c(CNC(=O)CN)cc3cc2Cl)on1] | 2-amino-N-((5-chloro-6-((3- methylisoxazol-5-yl)methoxy)-1H- indol-2-yl)methyl)acetamide |
| 618 | 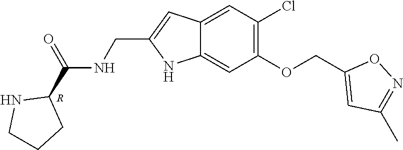 | (R)-N-((5-chloro-6-((3-methylisoxazol- 5-yl)methoxy)-1H-indol-2- yl)methyl)pyrrolidine-2-carboxamide |
| 619 | 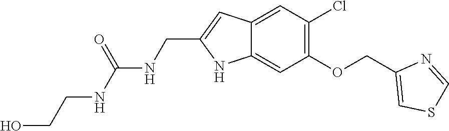 [SMILES: O=C(NCCO)NCc1cc2cc(Cl)c(OCc3cscn3)cc2[nH]1] | 1-((5-chloro-6-(thiazol-4-ylmethoxy)- 1H-indol-2-yl)methyl)-3-(2- hydroxyethyl)urea |
| 620 | 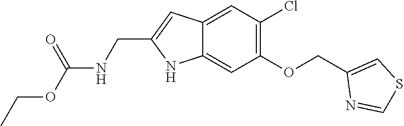 [SMILES: CCOC(=O)NCc1cc2cc(Cl)c(OCc3cscn3)cc2[nH]1] | ethyl ((5-chloro-6-(thiazol-4- ylmethoxy)-1H-indol-2- yl)methyl)carbamate |
| 621 | 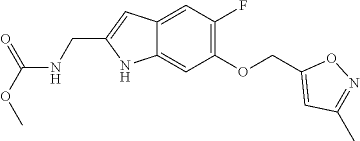 [SMILES: COC(=O)NCc1cc2cc(F)c(OCc3cc(C)no3)cc2[nH]1] | methyl ((5-fluoro-6-((3-methylisoxazol- 5-yl)methoxy)-1H-indol-2- yl)methyl)carbamate |
| 622 | 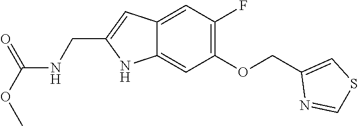 [SMILES: COC(=O)NCc1cc2cc(F)c(OCc3cscn3)cc2[nH]1] | methyl ((5-fluoro-6-(thiazol-4- ylmethoxy)-1H-indol-2- yl)methyl)carbamate |
| 623 | 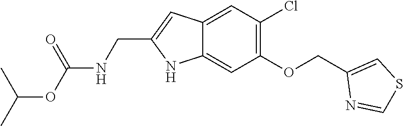 [SMILES: CC(C)OC(=O)NCc1cc2cc(Cl)c(OCc3cscn3)cc2[nH]1] | isopropyl ((5-chloro-6-(thiazol-4- ylmethoxy)-1H-indol-2- yl)methyl)carbamate |
| 624 | 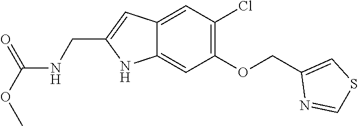 [SMILES: COC(=O)NCc1cc2cc(Cl)c(OCc3cscn3)cc2[nH]1] | methyl ((5-chloro-6-(thiazol-4- ylmethoxy)-1H-indol-2- yl)methyl)carbamate |
| 625 | 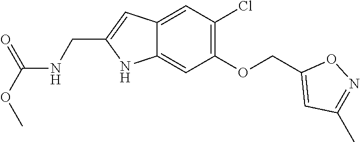 [SMILES: COC(=O)NCc1cc2cc(Cl)c(OCc3cc(C)no3)cc2[nH]1] | methyl ((5-chloro-6-((3- methylisoxazol-5-yl)methoxy)-1H- indol-2-yl)methyl)carbamate |
| 626 | 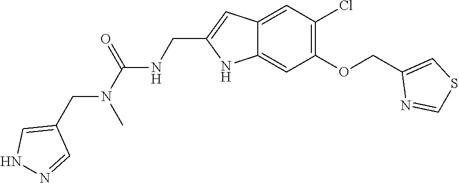 [SMILES: CN(Cc1cn[nH]c1)C(=O)NCc1cc2cc(Cl)c(OCc3cscn3)cc2[nH]1] | 1-((1H-pyrazol-4-yl)methyl)-3-((5- chloro-6-(thiazol-4-ylmethoxy)-1H- indol-2-yl)methyl)-1-methylurea |
| 627 | 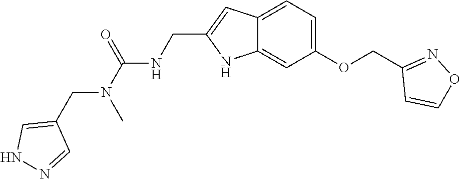 [SMILES: CN(Cc1cn[nH]c1)C(=O)NCc1cc2ccc(OCc3ccon3)cc2[nH]1] | 1-((1H-pyrazol-4-yl)methyl)-3-((6- (isoxazol-3-ylmethoxy)-1H-indol-2- yl)methyl)-1-methylurea |
| 628 | 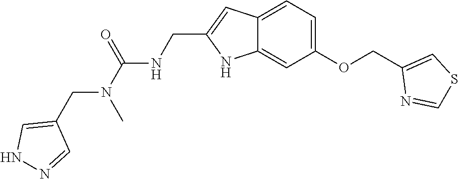 [SMILES: CN(Cc1cn[nH]c1)C(=O)NCc1cc2ccc(OCc3cscn3)cc2[nH]1] | 1-((1H-pyrazol-4-yl)methyl)-1-methyl- 3-((6-(thiazol-4-ylmethoxy)-1H-indol- 2-yl)methyl)urea |
| 629 | 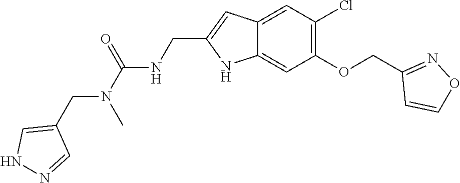 [SMILES: CN(Cc1cn[nH]c1)C(=O)NCc1cc2cc(Cl)c(OCc3ccon3)cc2[nH]1] | 1-((1H-pyrazol-4-yl)methyl)-3-((5- chloro-6-(isoxazol-3-ylmethoxy)-1H- indol-2-yl)methyl)-1-methylurea |
| 630 | 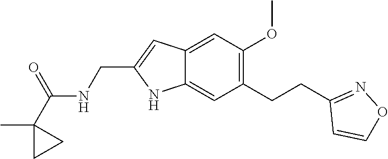 [SMILES: COc1cc2cc(CNC(=O)C3(C)CC3)[nH]c2cc1CCc1ccon1] | N-((6-(2-(isoxazol-3-yl)ethyl)-5- methoxy-1H-indol-2-yl)methyl)-1- methylcyclopropane-1-carboxamide |
| 631 | 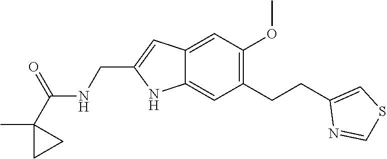 [SMILES: COc1cc2cc(CNC(=O)C3(C)CC3)[nH]c2cc1CCc1cscn1] | N-((5-methoxy-6-(2-(thiazol-4- yl)ethyl)-1H-indol-2-yl)methyl)-1- methylcyclopropane-1-carboxamide |
| 632 | 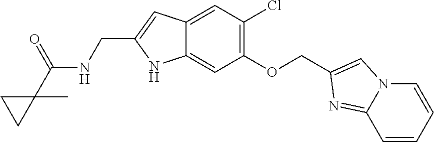 [SMILES: CC1(C(=O)NCc2cc3cc(Cl)c(OCc4cn5ccccc5n4)cc3[nH]2)CC1] | N-((5-chloro-6-(imidazo[1,2-a]pyridin- 2-ylmethoxy)-1H-indol-2-yl)methyl)-1- methylcyclopropane-1-carboxamide |
| 633 | 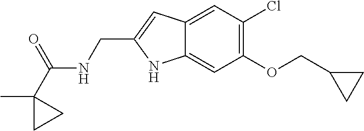 [SMILES: CC1(C(=O)NCc2cc3cc(Cl)c(OCC4CC4)cc3[nH]2)CC1] | N-((5-chloro-6-(cyclopropylmethoxy)- 1H-indol-2-yl)methyl)-1- methylcyclopropane-1-carboxamide |
| 634 | 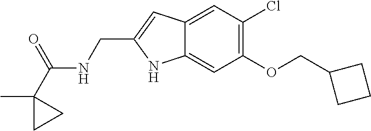 [SMILES: CC1(C(=O)NCc2cc3cc(Cl)c(OCC4CCC4)cc3[nH]2)CC1] | N-((5-chloro-6-(cyclobutylmethoxy)- 1H-indol-2-yl)methyl)-1- methylcyclopropane-1-carboxamide |
| 635 | 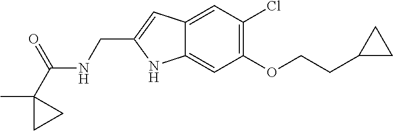 [SMILES: CC1(C(=O)NCc2cc3cc(Cl)c(OCCC4CC4)cc3[nH]2)CC1] | N-((5-chloro-6-(2-cyclopropylethoxy)- 1H-indol-2-yl)methyl)-1- methylcyclopropane-1-carboxamide |
| 636 | 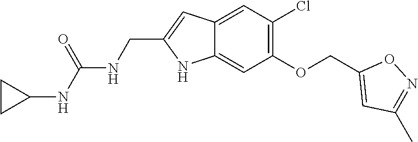 [SMILES: Cc1cc(COc2cc3[nH]c(CNC(=O)NC4CC4)cc3cc2Cl)on1] | 1-((5-chloro-6-((3-methylisoxazol-5- yl)methoxy)-1H-indol-2-yl) methyl)-3- cyclopropylurea |
| 637 | 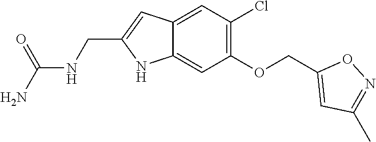 [SMILES: Cc1cc(COc2cc3[nH]c(CNC(N)=O)cc3cc2Cl)on1] | 1-((5-chloro-6-((3-methylisoxazol-5- yl)methoxy)-1H-indol-2-yl)methyl)urea |
| 638 | 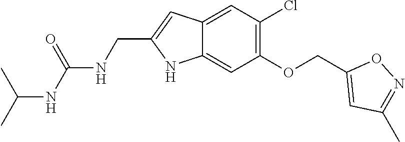 [SMILES: Cc1cc(COc2cc3[nH]c(CNC(=O)NC(C)C)cc3cc2Cl)on1] | 1-((5-chloro-6-((3-methylisoxazol-5- yl)methoxy)-1H-indol-2-yl)methyl)-3- isopropylurea |
| 639 | 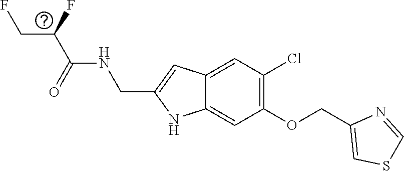 [SMILES: O=C(NCc1cc2cc(Cl)c(OCc3cscn3)cc2[nH]1)[C@H](F)CF] | (S)-N-((5-chloro-6-(thiazol-4- ylmethoxy)-1H-indol-2-yl)methyl)-2,3- difluoropropanamide and (R)-N-((5- chloro-6-(thiazol-4-ylmethoxy)-1H- indol-2-yl)methyl)-2,3- difluoropropanamide |
| 640 | 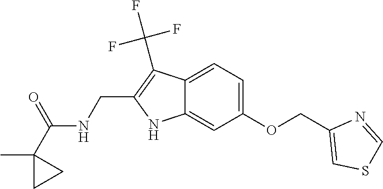 [SMILES: CC1(C(=O)NCc2[nH]c3cc(OCc4cscn4)ccc3c2C(F)(F)F)CC1] | 1-methyl-N-((6-(thiazol-4-ylmethoxy)- 3-(trifluoromethyl)-1H-indol-2- yl)methyl)cyclopropane-1-carboxamide |
| 641 | 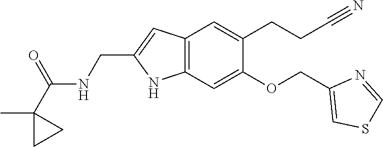 [SMILES: CC1(C(=O)NCc2cc3cc(CCC#N)c(OCc4cscn4)cc3[nH]2)CC1] | N-((5-(2-cyanoethyl)-6-(thiazol-4- ylmethoxy)-1H-indol-2-yl)methyl)-1- methylcyclopropane-1-carboxamide |
| 642 | 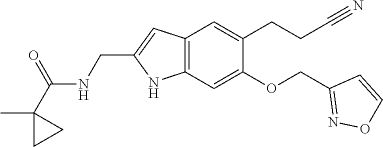 [SMILES: CC1(C(=O)NCc2cc3cc(CCC#N)c(OCc4ccon4)cc3[nH]2)CC1] | N-((5-(2-cyanoethyl)-6-(isoxazol-3- ylmethoxy)-1H-indol-2-yl)methyl)-1- methylcyclopropane-1-carboxamide |
| 643 | 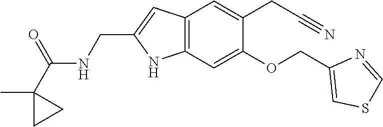 [SMILES: CC1(C(=O)NCc2cc3cc(CC#N)c(OCc4cscn4)cc3[nH]2)CC1] | N-((5-(cyanomethyl)-6-(thiazol-4- ylmethoxy)-1H-indol-2-yl)methyl)-1- methylcyclopropane-1-carboxamide |
| 644 | 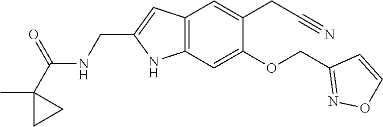 [SMILES: CC1(C(=O)NCc2cc3cc(CC#N)c(OCc4ccon4)cc3[nH]2)CC1] | N-((5-(cyanomethyl)-6-(isoxazol-3- ylmethoxy)-1H-indol-2-yl)methyl)-1- methylcyclopropane-1-carboxamide |
| 645 | 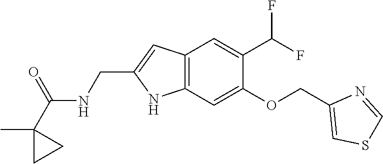 [SMILES: CC1(C(=O)NCc2cc3cc(C(F)F)c(OCc4cscn4)cc3[nH]2)CC1] | N-((5-(difluoromethyl)-6-(thiazol-4- ylmethoxy)-1H-indol-2-yl)methyl)-1- methylcyclopropane-1-carboxamide |
| 646 | 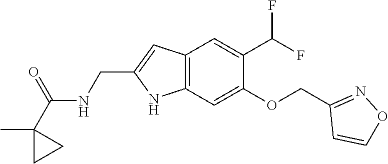 [SMILES: CC1(C(=O)NCc2cc3cc(C(F)F)c(OCc4ccon4)cc3[nH]2)CC1] | N-((5-(difluoromethyl)-6-(isoxazol-3- ylmethoxy)-1H-indol-2-yl)methyl)-1- methylcyclopropane-1-carboxamide |
| 647 | 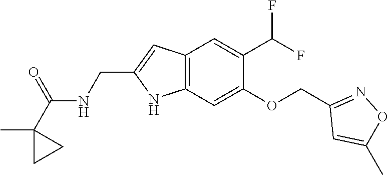 | N-((5-(difluoromethyl)-6-((5- methylisoxazol-3-yl)methoxy)-1H- indol-2-yl)methyl)-1- methylcyclopropane-1-carboxamide |
| 648 | 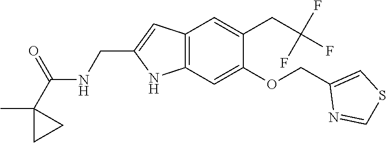 [SMILES: CC1(C(=O)NCc2cc3cc(CC(F)(F)F)c(OCc4cscn4)cc3[nH]2)CC1] | 1-methyl-N-((6-(thiazol-4-ylmethoxy)- 5-(2,2,2-trifluoroethyl)-1H-indol-2- yl)methyl)cyclopropane-1-carboxamide |
| 649 | 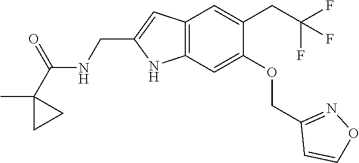 [SMILES: CC1(C(=O)NCc2cc3cc(CC(F)(F)F)c(OCc4ccon4)cc3[nH]2)CC1] | N-((6-(isoxazol-3-ylmethoxy)-5-(2,2,2- trifluoroethyl)-1H-indol-2-yl)methyl)- 1-methylcyclopropane-1-carboxamide |
| 650 | 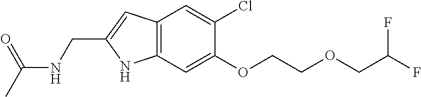 [SMILES: CC(=O)NCc1cc2cc(Cl)c(OCCOCC(F)F)cc2[nH]1] | N-((5-chloro-6-(2-(2,2- difluoroethoxy)ethoxy)-1H-indol-2- yl)methyl)acetamide |
| 651 | 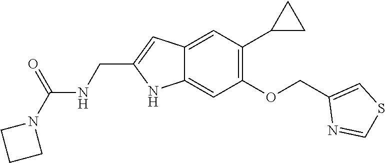 [SMILES: O=C(NCc1cc2cc(C3CC3)c(OCc3cscn3)cc2[nH]1)N1CCC1] | N-((5-cyclopropyl-6-(thiazol-4- ylmethoxy)-1H-indol-2- yl)methyl)azetidine-1-carboxamide |
| 652 | 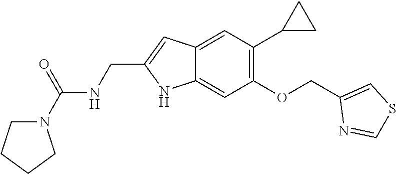 [SMILES: O=C(NCc1cc2cc(C3CC3)c(OCc3cscn3)cc2[nH]1)N1CCCC1] | N-((5-cyclopropyl-6-(thiazol-4- ylmethoxy)-1H-indol-2- yl)methyl)pyrrolidine-1-carboxamide |
| 653 | 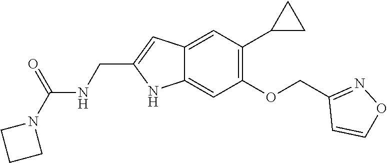 [SMILES: O=C(NCc1cc2cc(C3CC3)c(OCc3ccon3)cc2[nH]1)N1CCC1] | N-((5-cyclopropyl-6-(isoxazol-3- ylmethoxy)-1H-indol-2- yl)methyl)azetidine-1-carboxamide |
| 654 | 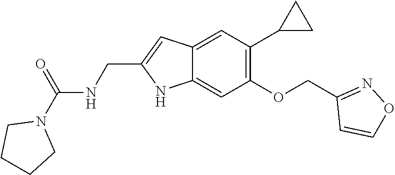 [SMILES: O=C(NCc1cc2cc(C3CC3)c(OCc3ccon3)cc2[nH]1)N1CCCC1] | N-((5-cyclopropyl-6-(isoxazol-3- ylmethoxy)-1H-indol-2- yl)methyl)pyrrolidine-1-carboxamide |
| 655 | 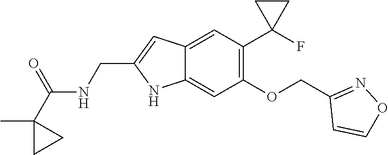 [SMILES: CC1(C(=O)NCc2cc3cc(C4(F)CC4)c(OCc4ccon4)cc3[nH]2)CC1] | N-((5-(1-fluorocyclopropyl)-6- (isoxazol-3-ylmethoxy)-1H-indol-2- yl)methyl)-1-methylcyclopropane-1- carboxamide |
| 656 | 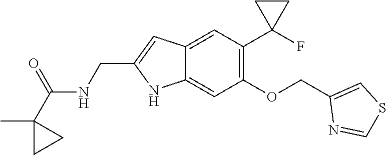 [SMILES: CC1(C(=O)NCc2cc3cc(C4(F)CC4)c(OCc4cscn4)cc3[nH]2)CC1] | N-((5-(1-fluorocyclopropyl)-6-(thiazol- 4-ylmethoxy)-1H-indol-2-yl)methyl)-1- methylcyclopropane-1-carboxamide |
| 657 | 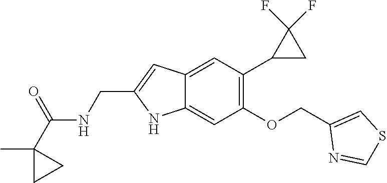 [SMILES: CC1(C(=O)NCc2cc3cc(C4CC4(F)F)c(OCc4cscn4)cc3[nH]2)CC1] | N-((5-(2,2-difluorocyclopropyl)-6- (thiazol-4-ylmethoxy)-1H-indol-2- yl)methyl)-1-methylcyclopropane-1- carboxamide |
| 658 | 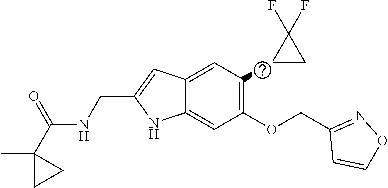 [SMILES: CC1(C(=O)NCc2cc3cc([C@@H]4CC4(F)F)c(OCc4ccon4)cc3[nH]2)CC1] | (S)-N-((5-(2,2-difluorocyclopropyl)-6- (isoxazol-3-ylmethoxy)-1H-indol-2- yl)methyl)-1-methylcyclopropane-1- carboxamide and (R)-N-((5-(2,2- difluorocyclopropyl)-6-(isoxazol-3- ylmethoxy)-1H-indol-2-yl)methyl)-1- methylcyclopropane-1-carboxamide |
| 659 | 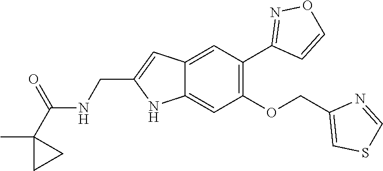 [SMILES: CC1(C(=O)NCc2cc3cc(-c4ccon4)c(OCc4cscn4)cc3[nH]2)CC1] | (5-chloro-6-(2-(isoxazol-3-yl)ethyl)- 1H-indol-2-yl)methanamine |
| 660 | 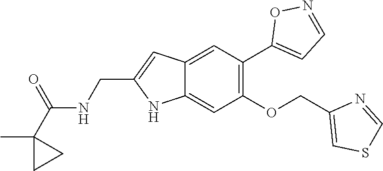 [SMILES: CC1(C(=O)NCc2cc3cc(-c4ccno4)c(OCc4cscn4)cc3[nH]2)CC1] | N-((5-(isoxazol-5-yl)-6-(thiazol-4- ylmethoxy)-1H-indol-2-yl)methyl)-1- methylcyclopropane-1-carboxamide |
| 661 | 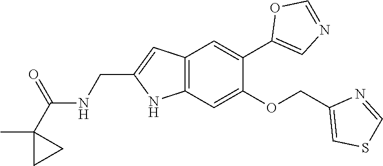 [SMILES: CC1(C(=O)NCc2cc3cc(-c4cnco4)c(OCc4cscn4)cc3[nH]2)CC1] | 1-methyl-N-((5-(oxazol-5-yl)-6- (thiazol-4-ylmethoxy)-1H-indol-2- yl)methyl)cyclopropane-1-carboxamide |
| 662 | 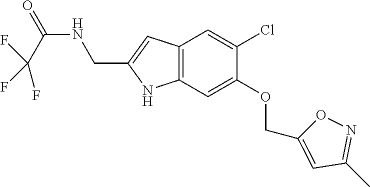 [SMILES: Cc1cc(COc2cc3[nH]c(CNC(=O)C(F)(F)F)cc3cc2Cl)on1] | N-((5-chloro-6-((3-methylisoxazol-5- yl)methoxy)-1H-indol-2-yl)methyl)- 2,2,2-trifluoroacetamide |
| 663 | 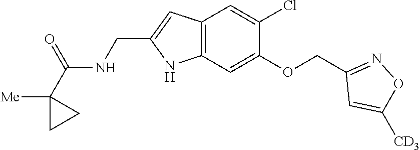 [SMILES: [2H]C([2H])([2H])c1cc(COc2cc3[nH]c(CNC(=O)C4(C)CC4)cc3cc2Cl)no1] | N-((5-chloro-6-((5-(methyl- d3)isoxazol-3-yl)methoxy)-1H-indol-2- yl)methyl)-1-methylcyclopropane-1- carboxamide |
| 664 | 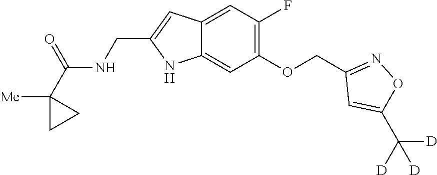 [SMILES: [2H]C([2H])([2H])c1cc(COc2cc3[nH]c(CNC(=O)C4(C)CC4)cc3cc2F)no1] | N-((5-fluoro-6-((5-(methyl-d3)isoxazol- 3-yl)methoxy)-1H-indol-2-yl)methyl)- 1-methylcyclopropane-1-carboxamide |
| 665 | 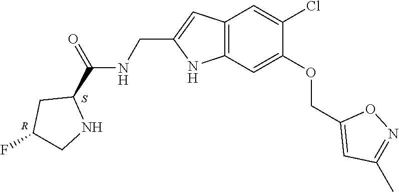 | (2S,4R)-N-((5-chloro-6-((3- methylisoxazol-5-yl)methoxy)-1H- indol-2-yl)methyl)-4-fluoropyrrolidine- 2-carboxamide |
| 666 | 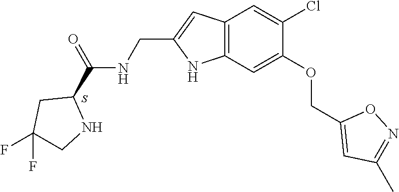 | (S)-N-((5-chloro-6-((3-methylisoxazol- 5-yl)methoxy)-1H-indol-2-yl)methyl)- 4,4-difluoropyrrolidine-2-carboxamide |
| 667 | 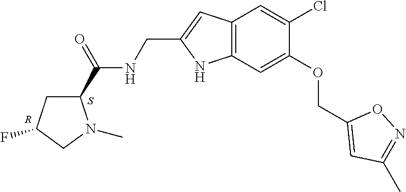 | (2S,4R)-N-((5-chloro-6-((3- methylisoxazol-5-yl)methoxy)-1H- indol-2-yl)methyl)-4-fluoro-1- methylpyrrolidine-2-carboxamide |
| 668 | 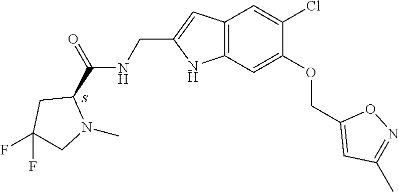 | (S)-N-((5-chloro-6-((3-methylisoxazol- 5-yl)methoxy)-1H-indol-2-yl)methyl)- 4,4-difluoro-1-methylpyrrolidine-2- carboxamide |
| 669 | 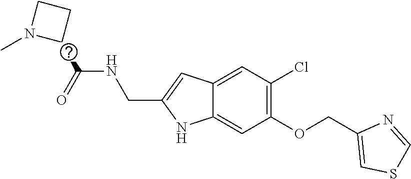 [SMILES: CN1CC[C@H]1C(=O)NCc1cc2cc(Cl)c(OCc3cscn3)cc2[nH]1] | (S)-N-((5-chloro-6-(thiazol-4- ylmethoxy)-1H-indol-2-yl)methyl)-1- methylazetidine-2-carboxamide and (R)-N-((5-chloro-6-(thiazol-4- ylmethoxy)-1H-indol-2-yl)methyl)-1- methylazetidine-2-carboxamide |

In some embodiments, provided herein is a compound of formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
N-((6-(benzylamino)-1H-indol-2-yl)methyl)propionamide;
N-((6-(benzyl(methyl)amino)-1H-indol-2-yl)methyl)-1-methylcyclopropane-1-carboxamide;
N-((6-(benzylamino)-1H-indol-2-yl)methyl)-1-methyl cyclopropanecarboxamide;
1-methyl-N-((6-((pyridin-2-ylmethyl)amino)-1H-indol-2-yl)methyl)cyclopropane-1-carboxamide;
N-((6-(benzyloxy)-1H-indol-2-yl)methyl)-1-methylcyclopropanecarboxamide;
N-((5-fluoro-6-(isoxazol-3-ylmethoxy)-1H-indol-2-yl) methyl)-1-methylcyclopropane-1-carboxamide;
N-((5-fluoro-6-(oxazol-4-ylmethoxy)-1H-indol-2-yl)methyl)-1-methylcyclopropane-1-carboxamide;
N-((5-fluoro-6-(thiazol-4-ylmethoxy)-1H-indol-2-yl)methyl)-1-methylcyclopropane-1-carboxamide;
N-((5-fluoro-6-(thiazol-5-ylmethoxy)-1H-indol-2-yl)methyl)-1-methylcyclopropane-1-carboxamide;
N-((6-(isoxazol-3-ylmethoxy)-5-methyl-1H-indol-2-yl)methyl)-1-methylcyclopropane-1-carboxamide;
1-methyl-N-((5-methyl-6-(thiazol-4-ylmethoxy)-1H-indol-2-yl)methyl)cyclopropane-1-carboxamide;
N-((5-fluoro-6-((3-methylisoxazol-5-yl)methoxy)-1H-indol-2-yl)methyl)-1-methylcyclopropane-1-carboxamide;
N-((5-fluoro-6-((2-methylthiazol-5-yl)methoxy)-1H-indol-2-yl)methyl)-1-methylcyclopropane-1-carboxamide;
1-methyl-N-((5-methyl-6-((5-methylisoxazol-3-yl)methoxy)-1H-indol-2-yl)methyl)cyclopropane-1-carboxamide;
N-((5-chloro-4-fluoro-6-(isoxazol-3-ylmethoxy)-1H-indol-2-yl)methyl)-1-methylcyclopropane-1-carboxamide;
N-((5-chloro-7-fluoro-6-(isoxazol-3-ylmethoxy)-1H-indol-2-yl)methyl)-1-methylcyclopropane-1-carboxamide;
N-((5-chloro-4-fluoro-6-(thiazol-4-ylmethoxy)-1H-indol-2-yl)methyl)-1-methylcyclopropane-1-carboxamide;
N-((5-chloro-7-fluoro-6-(thiazol-4-ylmethoxy)-1H-indol-2-yl)methyl)-1-methylcyclopropane-1-carboxamide;
N-((7-fluoro-6-(thiazol-4-ylmethoxy)-1H-indol-2-yl)methyl)-1-methylcyclopropane-1-carboxamide;
N-((6-(isoxazol-3-ylmethoxy)-1H-indol-2-yl)methyl)-1-methylcyclopropane-1-carboxamide;
N-((5-chloro-6-((5-methylpyridin-2-yl)methoxy)-1H-indol-2-yl)methyl)-1-methylcyclopropane-1-carboxamide;
N-((5-chloro-6-((4-methylpyridin-2-yl)methoxy)-1H-indol-2-yl)methyl)-1-methylcyclopropane-1-carboxamide;
N-((5-chloro-6-((5-methylpyridin-3-yl)methoxy)-1H-indol-2-yl)methyl)-1-methylcyclopropane-1-carboxamide;
N-((5-chloro-6-((4-methylthiazol-2-yl)methoxy)-1H-indol-2-yl)methyl)-1-methylcyclopropane-1-carboxamide;
N-((5-chloro-6-(thiophen-3-ylmethoxy)-1H-indol-2-yl)methyl)-1-methylcyclopropane-1-carboxamide;
N-((5-chloro-6-((3-fluoropyridin-2-yl)methoxy)-1H-indol-2-yl)methyl)-1-methylcyclopropane-1-carboxamide;
N-((4-fluoro-6-(isoxazol-3-ylmethoxy)-1H-indol-2-yl)methyl)-1-methylcyclopropane-1-carboxamide;
N-((5-chloro-6-((5-fluoropyridin-2-yl)methoxy)-1H-indol-2-yl)methyl)-1-methylcyclopropane-1-carboxamide;
N-((4-fluoro-6-(thiazol-4-ylmethoxy)-1H-indol-2-yl)methyl)-1-methylcyclopropane-1-carboxamide;
N-((5-chloro-6-((2-fluoropyridin-3-yl)methoxy)-1H-indol-2-yl)methyl)-1-methylcyclopropane-1-carboxamide;
N-((5-chloro-6-((6-fluoropyridin-3-yl)methoxy)-1H-indol-2-yl)methyl)-1-methylcyclopropane-1-carboxamide;
N-((5-chloro-6-((6-fluoropyridin-2-yl)methoxy)-1H-indol-2-yl)methyl)-1-methylcyclopropane-1-carboxamide;
N-((6-((6-fluoropyridin-2-yl)methoxy)-1H-indol-2-yl)methyl)-1-methylcyclopropane-1-carboxamide;
N-((6-((6-fluoropyridin-3-yl)methoxy)-1H-indol-2-yl)methyl)-1-methylcyclopropane-1-carboxamide;
N-((5-chloro-6-((4-fluoropyridin-2-yl)methoxy)-1H-indol-2-yl)methyl)-1-methylcyclopropane-1-carboxamide;
N-((5-chloro-6-((5-fluoropyridin-3-yl)methoxy)-1H-indol-2-yl)methyl)-1-methylcyclopropane-1-carboxamide;
N-((5-chloro-6-((6-methylpyridin-3-yl)methoxy)-1H-indol-2-yl)methyl)-1-methylcyclopropane-1-carboxamide;
N-((5-chloro-6-(isothiazol-3-ylmethoxy)-1H-indol-2-yl)methyl)-1-methylcyclopropane-1-carboxamide;
N-((5-chloro-6-(pyridin-3-ylmethoxy)-1H-indol-2-yl)methyl)-1-methylcyclopropane-1-carboxamide;
N-((5-chloro-6-(thiazol-2-ylmethoxy)-1H-indol-2-yl)methyl)-1-methylcyclopropane-1-carboxamide;
N-((5-chloro-6-(pyridin-2-ylmethoxy)-1H-indol-2-yl)methyl)-1-methylcyclopropane-1-carboxamide;
N-((5-chloro-6-((6-methylpyridin-2-yl)methoxy)-1H-indol-2-yl)methyl)-1-methylcyclopropane-1-carboxamide;
1-methyl-N-((6-(thiazol-2-ylmethoxy)-1H-indol-2-yl)methyl)cyclopropane-1-carboxamide;
N-((6-((4-chlorobenzyl)oxy)-1H-indol-2-yl)methyl)-1-methylcyclopropane-1-carboxamide;
N-((5-chloro-6-((5-methylisoxazol-3-yl)methoxy)-1H-indol-2-yl)methyl)-1-methylcyclopropane-1-carboxamide;
N-((5-chloro-6-(isoxazol-3-ylmethoxy)-1H-indol-2-yl)methyl)-1-methylcyclopropane-1-carboxamide;
N-((6-((5-methylisoxazol-3-yl)methoxy)-1H-indol-2-yl)methyl)propionamide;
1-methyl-N-((6-(thiazol-5-ylmethoxy)-1H-indol-2-yl)methyl)cyclopropane-1-carboxamide;
1-methyl-N-((6-(oxazol-4-ylmethoxy)-1H-indol-2-yl)methyl)cyclopropane-1-carboxamide;
1-methyl-N-((6-((5-methylisoxazol-3-yl)methoxy)-1H-indol-2-yl)methyl)cyclopropane-1-carboxamide;
1-methyl-N-((6-(pyrimidin-2-ylmethoxy)-1H-indol-2-yl)methyl)cyclopropane-1-carboxamide;
1-methyl-N-((6-(pyrimidin-5-ylmethoxy)-1H-indol-2-yl)methyl)cyclopropane-1-carboxamide;
1-methyl-N-((6-(pyrimidin-4-ylmethoxy)-1H-indol-2-yl)methyl)cyclopropane-1-carboxamide;
1-methyl-N-((6-(pyridin-2-ylmethoxy)-1H-indol-2-yl)methyl)cyclopropane-1-carboxamide;
1-methyl-N-((6-(pyridin-3-ylmethoxy)-1H-indol-2-yl)methyl)cyclopropane-1-carboxamide;
1-methyl-N-((6-(pyridin-4-ylmethoxy)-1H-indol-2-yl)methyl)cyclopropane-1-carboxamide;
N-((5-fluoro-6-(oxazol-5-ylmethoxy)-1H-indol-2-yl) methyl)-1-methylcyclopropane-1-carboxamide;
1-methyl-N-((6-((5-methylisoxazol-3-yl)methoxy)-5-(trifluoromethyl)-1H-indol-2-yl)methyl)cyclopropanecarboxamide;
N-((6-(isoxazol-3-ylmethoxy)-5-(trifluoromethyl)-1H-indol-2-yl)methyl)-1-methylcyclopropane-1-carboxamide;
1-methyl-N-((6-(thiazol-4-ylmethoxy)-5-(trifluoromethyl)-1H-indol-2-yl)methyl)cyclopropane-1-carboxamide;
N-((5-ethyl-6-(isoxazol-3-ylmethoxy)-1H-indol-2-yl)methyl)-1-methylcyclopropane-1-carboxamide;
N-((6-(isoxazol-5-ylmethoxy)-1H-indol-2-yl)methyl)-1-methylcyclopropanecarboxamide;
N-((5-chloro-6-((5-methylthiazol-2-yl)methoxy)-1H-indol-2-yl)methyl)-1-methylcyclopropane-1-carboxamide;
N-((5-chloro-6-(furan-3-ylmethoxy)-1H-indol-2-yl)methyl)-1-methylcyclopropane-1-carboxamide;
N-((5-chloro-6-(thiophen-2-ylmethoxy)-1H-indol-2-yl)methyl)-1-methylcyclopropane-1-carboxamide;
N-((5-chloro-6-(furan-2-ylmethoxy)-1H-indol-2-yl)methyl)-1-methylcyclopropane-1-carboxamide;
N-((5-chloro-6-(2-(thiazol-4-yl)ethoxy)-1H-indol-2-yl)methyl)-1-methylcyclopropane-1-carboxamide;
N-((6-(isothiazol-4-ylmethoxy)-1H-indol-2-yl)methyl)-1-methylcyclopropane-1-carboxamide;
N-((5-chloro-6-(isothiazol-4-ylmethoxy)-1H-indol-2-yl)methyl)-1-methylcyclopropane-1-carboxamide;
N-((5-chloro-6-((5-methyloxazol-2-yl)methoxy)-1H-indol-2-yl)methyl)-1-methylcyclopropane-1-carboxamide;
N-((5-chloro-6-((4-methyloxazol-2-yl)methoxy)-1H-indol-2-yl)methyl)-1-methylcyclopropane-1-carboxamide;
N-((5-fluoro-6-((5-(trifluoromethyl)isoxazol-3-yl)methoxy)-1H-indol-2-yl)methyl)-1-methylcyclopropane-1-carboxamide;
N-((5-fluoro-6-((5-methyloxazol-2-yl)methoxy)-1H-indol-2-yl)methyl)-1-methylcyclopropane-1-carboxamide;
N-((5-fluoro-6-((5-methyloxazol-2-yl)methoxy)-1H-indol-2-yl)methyl)-1-methylcyclopropane-1-carboxamide;
N-((6-(2-(isoxazol-3-yl)ethyl)-1H-indol-2-yl)methyl)-1-methylcyclopropanecarboxamide;
1-methyl-N-((6-(2-(thiazol-4-yl)ethyl)-1H-indol-2-yl)methyl)cyclopropane-1-carboxamide;
1-methyl-N-((6-(2-(5-methylisoxazol-3-yl)ethyl)-1H-indol-2-yl)methyl)cyclopropane-1-carboxamide;
1-methyl-N-((6-((5-methylisoxazol-3-yl)oxy)-1H-indol-2-yl)methyl)cyclopropanecarboxamide;
N-((5-chloro-6-(isoxazol-3-ylmethoxy)-1H-indol-2-yl)methyl)azetidine-1-carboxamide;
N-((5-chloro-6-(thiazol-4-ylmethoxy)-1H-indol-2-yl)methyl)-3-methylazetidine-1-carboxamide;
N-((5-chloro-6-(thiazol-4-ylmethoxy)-1H-indol-2-yl)methyl)azetidine-1-carboxamide;
N-((5-chloro-6-(thiazol-4-ylmethoxy)-1H-indol-2-yl)methyl)pyrrolidine-1-carboxamide;
N-((5-chloro-6-((3-methylisoxazol-5-yl)methoxy)-1H-indol-2-yl)methyl)pyrrolidine-1-carboxamide;
N-((5-chloro-6-(isoxazol-3-ylmethoxy)-1H-indol-2-yl)methyl)morpholine-4-carboxamide;
N-((5-chloro-6-(isoxazol-3-ylmethoxy)-1H-indol-2-yl)methyl)piperidine-1-carboxamide;
N-((5-chloro-6-(isoxazol-3-ylmethoxy)-1H-indol-2-yl)methyl)-2-methylpyrrolidine-1-carboxamide;
N-((5-chloro-6-(isoxazol-3-ylmethoxy)-1H-indol-2-yl)methyl)-3-methylpyrrolidine-1-carboxamide;
N-((5-fluoro-6-(isoxazol-3-ylmethoxy)-1H-indol-2-yl)methyl)piperidine-1-carboxamide;
N-((5-fluoro-6-(isoxazol-3-ylmethoxy)-1H-indol-2-yl)methyl)azetidine-1-carboxamide;
N-((5-fluoro-6-(isoxazol-3-ylmethoxy)-1H-indol-2-yl)methyl)-2-methylpyrrolidine-1-carboxamide;
N-((5-fluoro-6-(isoxazol-3-ylmethoxy)-1H-indol-2-yl)methyl)-3-methylpyrrolidine-1-carboxamide;
N-((5-fluoro-6-(thiazol-4-ylmethoxy)-1H-indol-2-yl)methyl)azetidine-1-carboxamide;
N-((5-chloro-6-(thiazol-4-ylmethoxy)-1H-indol-2-yl)methyl)-3-fluoropyrrolidine-1-carboxamide;
N-((6-(isothiazol-3-ylmethoxy)-1H-indol-2-yl)methyl)-1-methylcyclopropanecarboxamide; (N-((5-chloro-6-((5-ethylisoxazol-3-yl)methoxy)-1H-indol-2-yl)methyl)-1-methylcyclopropanecarboxamide;
N-((5-chloro-6-((5-(difluoromethyl)isoxazol-3-yl)methoxy)-1H-indol-2-yl)methyl)-1-methylcyclopropane-1-carboxamide;
N-((6-((5-(difluoromethyl)isoxazol-3-yl)methoxy)-1H-indol-2-yl)methyl)-1-methylcyclopropane-1-carboxamide;
N-((6-((2-fluoropyridin-3-yl)methoxy)-1H-indol-2-yl)methyl)-1-methylcyclopropane-1-carboxamide formate;
N-((5-chloro-6-(2-(5-methylisoxazol-3-yl)ethoxy)-1H-indol-2-yl)methyl)-1-methylcyclopropane-1-carboxamide;
N-((5-chloro-6-((5-cyclopropylisoxazol-3-yl)methoxy)-1H-indol-2-yl)methyl)-1-methylcyclopropane-1-carboxamide;
N-((5-chloro-6-((6-(trifluoromethyl)pyridin-2-yl)methoxy)-1H-indol-2-yl)methyl)-1-methylcyclopropane-1-carboxamide;
N-((5-chloro-6-((5-isopropylisoxazol-3-yl)methoxy)-1H-indol-2-yl)methyl)-1-methylcyclopropane-1-carboxamide;
N-((5-chloro-6-(2-(isoxazol-3-yl)ethoxy)-1H-indol-2-yl)methyl)-1-methylcyclopropane-1-carboxamide;
N-((6-(isoxazol-3-ylmethoxy)-1H-indol-2-yl)methyl)pyrrolidine-1-carboxamide;
N-((6-(oxazol-4-ylmethoxy)-1H-indol-2-yl)methyl)pyrrolidine-1-carboxamide;
N-((6-(thiazol-5-ylmethoxy)-1H-indol-2-yl)methyl)pyrrolidine-1-carboxamide;
1-((6-(isoxazol-3-ylmethoxy)-1H-indol-2-yl)methyl)-3-methylurea;
1-methyl-3-((6-(oxazol-4-ylmethoxy)-1H-indol-2-yl)methyl)urea;
1-((5-chloro-6-(thiazol-5-ylmethoxy)-1H-indol-2-yl)methyl)-3-methylurea;
1-((5-chloro-6-(isoxazol-3-ylmethoxy)-1H-indol-2-yl)methyl)-3-methylurea;
1-((5-chloro-6-(oxazol-4-ylmethoxy)-1H-indol-2-yl)methyl)-3-methylurea;
N-((5-chloro-6-(isoxazol-3-ylmethoxy)-1H-indol-2-yl)methyl)pyrrolidine-1-carboxamide;
N-((5-chloro-6-(oxazol-4-ylmethoxy)-1H-indol-2-yl)methyl)pyrrolidine-1-carboxamide;
N-((5-fluoro-6-(thiazol-4-ylmethoxy)-1H-indol-2-yl)methyl)pyrrolidine-1-carboxamide;
N-((5-fluoro-6-(oxazol-4-ylmethoxy)-1H-indol-2-yl)methyl)pyrrolidine-1-carboxamide;
N-((5-fluoro-6-(isoxazol-3-ylmethoxy)-1H-indol-2-yl)methyl)pyrrolidine-1-carboxamide;
N-((5-fluoro-6-((5-methylisoxazol-3-yl)methoxy)-1H-indol-2-yl)methyl)pyrrolidine-1-carboxamide;
N-((5-fluoro-6-((3-methylisoxazol-5-yl)methoxy)-1H-indol-2-yl)methyl)pyrrolidine-1-carboxamide;
N-((5-chloro-6-(thiazol-5-ylmethoxy)-1H-indol-2-yl) methyl) pyrrolidine-1-carboxamide;
N-((5-chloro-6-((5-methyloxazol-2-yl)methoxy)-1H-indol-2-yl)methyl)pyrrolidine-1-carboxamide;
N-((6-(isoxazol-3-ylmethoxy)-1H-indol-2-yl)methyl)-3-methylpyrrolidine-1-carboxamide;
N-((6-(isoxazol-3-ylmethoxy)-1H-indol-2-yl)methyl)azetidine-1-carboxamide;
N-((6-(isoxazol-3-ylmethoxy)-1H-indol-2-yl)methyl)piperidine-1-carboxamide;
N-((6-(isoxazol-3-ylmethoxy)-1H-indol-2-yl)methyl)-2-methylpyrrolidine-1-carboxamide;
N-((5-chloro-6-((5-methylisoxazol-3-yl)methoxy)-1H-indol-2-yl)methyl)-1-(trifluoromethyl)cyclopropane-1-carboxamide;
N-((5-fluoro-6-(thiazol-4-ylmethoxy)-1H-indol-2-yl)methyl)propionamide;
N-((5-fluoro-6-(thiazol-4-ylmethoxy)-1H-indol-2-yl)methyl)-1-(trifluoromethyl)cyclopropane-1-carboxamide;
N-((5-chloro-6-((5-methylisoxazol-3-yl)methoxy)-1H-indol-2-yl)methyl)propionamide;
N-((5-chloro-6-(isoxazol-3-ylmethoxy)-1H-indol-2-yl)methyl)propionamide;
N-((5-chloro-6-((5-methylisoxazol-3-yl)methoxy)-1H-indol-2-yl)methyl)isobutyramide;
N-((5-chloro-6-(isoxazol-3-ylmethoxy)-1H-indol-2-yl)methyl)cyclopropanecarboxamide;
N-((5-chloro-6-(isoxazol-3-ylmethoxy)-1H-indol-2-yl)methyl)isobutyramide;
N-((5-chloro-6-((5-methylisoxazol-3-yl)methoxy)-1H-indol-2-yl)methyl)cyclopropanecarboxamide;
N-((5-chloro-6-(isoxazol-3-ylmethoxy)-1H-indol-2-yl)methyl)-1-(trifluoromethyl)cyclopropane-1-carboxamide;
N-((5-fluoro-6-(thiazol-4-ylmethoxy)-1H-indol-2-yl)methyl)isobutyramide;
N-((5-fluoro-6-(thiazol-4-ylmethoxy)-1H-indol-2-yl)methyl)cyclopropanecarboxamide;
N-((5-fluoro-6-(isoxazol-3-ylmethoxy)-1H-indol-2-yl)methyl)propionamide;
N-((5-fluoro-6-(isoxazol-3-ylmethoxy)-1H-indol-2-yl)methyl)isobutyramide;
N-((5-fluoro-6-(isoxazol-3-ylmethoxy)-1H-indol-2-yl)methyl)-1-(trifluoromethyl)cyclopropane-1-carboxamide;
N-((5-fluoro-6-(isoxazol-3-ylmethoxy)-1H-indol-2-yl)methyl)cyclopropanecarboxamide;
N-((5-chloro-6-(thiazol-4-ylmethoxy)-1H-indol-2-yl)methyl)acetamide;
N-((5-chloro-6-((5-methylisoxazol-3-yl)methoxy)-1H-indol-2-yl)methyl)acetamide;
N-((5-chloro-6-(isoxazol-3-ylmethoxy)-1H-indol-2-yl)methyl)acetamide;
N-((5-chloro-6-(thiazol-4-ylmethoxy)-1H-indol-2-yl)methyl)-1-(trifluoromethyl)cyclopropane-1-carboxamide;
N-((5-chloro-6-(thiazol-4-ylmethoxy)-1H-indol-2-yl)methyl)isobutyramide;
N-((5-chloro-6-(thiazol-4-ylmethoxy)-1H-indol-2-yl)methyl)propionamide;
N-((5-chloro-6-(thiazol-4-ylmethoxy)-1H-indol-2-yl)methyl)cyclopropanecarboxamide;
N-((5-chloro-6-(hydroxymethyl)-1H-indol-2-yl)methyl)-1-methylcyclopropanecarboxamide;
N-((5-chloro-6-(2-(thiazol-4-yl)ethyl)-1H-indol-2-yl)methyl)-1-methylcyclopropane-1-carboxamide;
N-((5-chloro-6-(2-(isoxazol-3-yl)ethyl)-1H-indol-2-yl)methyl)-1-methylcyclopropane-1-carboxamide;
N-((5-chloro-6-((isoxazol-3-ylmethyl)amino)-1H-indol-2-yl)methyl)-1-methylcyclopropane-1-carboxamide;
N-((6-((isoxazol-3-ylmethyl)amino)-1H-indol-2-yl)methyl)-1-methylcyclopropane-1-carboxamide;
1-methyl-N-((6-(1-(5-methylisoxazol-3-yl) propan-2-yl)-1H-indol-2-yl) methyl) cyclopropane-1-carboxamide;
trans-1-methyl-N-((6-(2-phenylcyclopropyl)-1H-indol-2-yl)methyl)cyclopropane-1-carboxamide;
N-((6-(isoxazol-3-ylmethoxy)-3-methyl-1H-indol-2-yl)methyl)-1-methylcyclopropane-1-carboxamide;
N-((6-(benzyloxy)-3-methyl-1H-indol-2-yl)methyl)-1-methylcyclopropane-1-carboxamide;
N-((3-chloro-6-(isoxazol-3-ylmethoxy)-1H-indol-2-yl)methyl)-1-methylcyclopropane-1-carboxamide;
N-((6-(benzyloxy)-3-chloro-1H-indol-2-yl)methyl)-1-methylcyclopropane-1-carboxamide;
N-((5-chloro-6-(isoxazol-3-ylmethyl)-1H-indol-2-yl)methyl)-1-methylcyclopropanecarboxamide;
1-methyl-N-((6-((5-methylisoxazol-3-yl)methyl)-1H-indol-2-yl)methyl)cyclopropanecarboxamide;
N-((5-chloro-6-(oxazol-4-ylmethoxy)-1H-indol-2-yl)methyl)-1-methylcyclopropane-1-carboxamide;
1-methyl-N-((6-(2-(methylamino)-2-oxoethoxy)-1H-indol-2-yl)methyl)cyclopropane-1-carboxamide;
1-methyl-N-((6-(oxazol-5-ylmethoxy)-1H-indol-2-yl)methyl)cyclopropane-1-carboxamide;
1-methyl-N-((6-(thiazol-4-ylmethoxy)-1H-indol-2-yl)methyl)cyclopropane-1-carboxamide;
1-methyl-N-((6-((5-methyl-1,3,4-thiadiazol-2-yl)methoxy)-1H-indol-2-yl)methyl)cyclopropane-1-carboxamide;
N-((6-((1,2,5-thiadiazol-3-yl)methoxy)-1H-indol-2-yl)methyl)-1-methylcyclopropane-1-carboxamide;
1-methyl-N-((6-((5-methyl-1,3,4-oxadiazol-2-yl)methoxy)-1H-indol-2-yl)methyl)cyclopropane-1-carboxamide;
1-methyl-N-((6-((1-methyl-1H-1,2,3-triazol-4-yl)methoxy)-1H-indol-2-yl)methyl)cyclopropane-1-carboxamide;
1-methyl-N-((6-((3-methyl-1,2,4-oxadiazol-5-yl)methoxy)-1H-indol-2-yl)methyl)cyclopropane-1-carboxamide;
N-((6-((1,2,3-thiadiazol-4-yl)methoxy)-1H-indol-2-yl)methyl)-1-methylcyclopropane-1-carboxamide;
N-((6-((1,3,4-thiadiazol-2-yl)methoxy)-1H-indol-2-yl)methyl)-1-methylcyclopropane-1-carboxamide;
N-((5-chloro-6-((2-methyl-2H-1,2,3-triazol-4-yl)methoxy)-1H-indol-2-yl)methyl)-1-methylcyclopropane-1-carboxamide;
1-methyl-N-((6-((2-methyl-2H-1,2,3-triazol-4-yl)methoxy)-1H-indol-2-yl)methyl)cyclopropane-1-carboxamide;
1-methyl-N-((6-((2-methyl-2H-tetrazol-5-yl)methoxy)-1H-indol-2-yl)methyl)cyclopropane-1-carboxamide;
N-((5-chloro-6-((3-methylisoxazol-5-yl)methoxy)-1H-indol-2-yl)methyl)-1-methylcyclopropane-1-carboxamide;
N-((5-chloro-6-((2-methylthiazol-5-yl)methoxy)-1H-indol-2-yl)methyl)-1-methylcyclopropane-1-carboxamide;
N-((5-chloro-6-(thiazol-5-ylmethoxy)-1H-indol-2-yl)methyl)-1-methylcyclopropane-1-carboxamide;
1-methyl-N-((6-((2-methylthiazol-5-yl)methoxy)-1H-indol-2-yl)methyl)cyclopropane-1-carboxamide;
1-methyl-N-((6-((2-methylthiazol-4-yl)methoxy)-1H-indol-2-yl)methyl)cyclopropane-1-carboxamide;
1-methyl-N-((6-((2-methyloxazol-4-yl)methoxy)-1H-indol-2-yl)methyl)cyclopropane-1-carboxamide;
1-methyl-N-((6-((3-methylisoxazol-5-yl)methoxy)-1H-indol-2-yl)methyl)cyclopropane-1-carboxamide;
N-((5-chloro-6-((2-methyloxazol-4-yl)methoxy)-1H-indol-2-yl)methyl)-1-methylcyclopropane-1-carboxamide;
N-((6-((1,2,5-thiadiazol-3-yl)methoxy)-5-chloro-1H-indol-2-yl)methyl)-1-methylcyclopropane-1-carboxamide;
N-((5-chloro-6-(thiazol-4-ylmethoxy)-1H-indol-2-yl)methyl)-1-methylcyclopropane-1-carboxamide;
N-((6-((5-ethylisoxazol-3-yl)methoxy)-1H-indol-2-yl)methyl)-1-methylcyclopropane-1-carboxamide;
N-((5-isopropyl-6-(thiazol-4-ylmethoxy)-1H-indol-2-yl)methyl)-1-methylcyclopropane-1-carboxamide;
N-((6-(cyclopentylmethoxy)-1H-indol-2-yl)methyl)-1-methylcyclopropane-1-carboxamide;
1-methyl-N-((6-((tetrahydrofuran-3-yl)methoxy)-1H-indol-2-yl)methyl)cyclopropane-1-carboxamide;
N-((6-(bicyclo[1.1.1]pentan-1-ylmethoxy)-1H-indol-2-yl)methyl)-1-methylcyclopropane-1-carboxamide;
N-((6-(2-(isoxazol-3-yl)ethoxy)-1H-indol-2-yl)methyl)-1-methylcyclopropane-1-carboxamide;
1-methyl-N-((6-(2-(thiazol-4-yl)ethoxy)-1H-indol-2-yl)methyl)cyclopropane-1-carboxamide;
N-((6-(benzofuran-6-ylmethoxy)-1H-indol-2-yl)methyl)-1-methylcyclopropane-1-carboxamide;
N-((6-(2-(bicyclo[1.1.1]pentan-1-yl)ethoxy)-1H-indol-2-yl)methyl)-1-methylcyclopropane-1-carboxamide;
N-((6-((1H-1,2,3-triazol-4-yl)methoxy)-1H-indol-2-yl)methyl)-1-methylcyclopropane-1-carboxamide;
1-methyl-N-((6-(2-(5-methylisoxazol-3-yl)propyl)-1H-indol-2-yl)methyl)cyclopropanecarboxamide;
N-((6-((isoxazol-3-ylmethyl)thio)-1H-indol-2-yl)methyl)-1-methylcyclopropanecarboxamide;
N-((5-chloro-6-((isoxazol-3-ylmethyl)thio)-1H-indol-2-yl)methyl)-1-methylcyclopropanecarboxamide;
N-((5-chloro-6-((3-hydroxybenzyl) oxy)-1H-indol-2-yl) methyl)-1-methylcyclopropane-1-carboxamide;
N-((6-(isoxazol-3-ylmethoxy)-1H-indol-2-yl)methyl)morpholine-4-carboxamide;
3-fluoro-N-((6-(isoxazol-3-ylmethoxy)-1H-indol-2-yl)methyl)pyrrolidine-1-carboxamide;
N-((7-fluoro-6-(isoxazol-3-ylmethoxy)-1H-indol-2-yl)methyl)-1-methylcyclopropane-1-carboxamide;
N-((5-ethyl-6-(thiazol-4-ylmethoxy)-1H-indol-2-yl)methyl)-1-methylcyclopropane-1-carboxamide;
N-((5-chloro-6-((2-hydroxybenzyl)oxy)-1H-indol-2-yl)methyl)-1-methylcyclopropane-1-carboxamide;
N-((5-isopropyl-6-(isoxazol-3-ylmethoxy)-1H-indol-2-yl)methyl)-1-methylcyclopropane-1-carboxamide;
N-((5-fluoro-6-((5-methylisoxazol-3-yl)methoxy)-1H-indol-2-yl)methyl)acetamide;
N-((5-chloro-6-(oxazol-4-ylmethoxy)-1H-indol-2-yl)methyl)acetamide;
N-((5-fluoro-6-(isoxazol-3-ylmethoxy)-1H-indol-2-yl)methyl)acetamide;
N-((5-fluoro-6-(thiazol-4-ylmethoxy)-1H-indol-2-yl)methyl)acetamide;
1-((5-chloro-6-(thiazol-4-ylmethoxy)-1H-indol-2-yl)methyl)-3-methylurea;
1-((5-fluoro-6-((3-methylisoxazol-5-yl)methoxy)-1H-indol-2-yl)methyl)-3-methylurea;
1-((5-chloro-6-((3-methylisoxazol-5-yl)methoxy)-1H-indol-2-yl)methyl)-3-methylurea;
1-((5-fluoro-6-((5-methylisoxazol-3-yl)methoxy)-1H-indol-2-yl)methyl)-3-methylurea;
1-((5-chloro-6-((5-methylisoxazol-3-yl)methoxy)-1H-indol-2-yl)methyl)-3-methylurea;
1-((5-fluoro-6-(thiazol-4-ylmethoxy)-1H-indol-2-yl)methyl)-3-methylurea;
1-((5-fluoro-6-(oxazol-4-ylmethoxy)-1H-indol-2-yl)methyl)-3-methylurea;
1-((5-fluoro-6-(isoxazol-3-ylmethoxy)-1H-indol-2-yl)methyl)-3-methylurea;
1-methyl-3-((6-(thiazol-4-ylmethoxy)-1H-indol-2-yl)methyl)urea;
N-((5-chloro-6-((3-methylisoxazol-5-yl)methoxy)-1H-indol-2-yl)methyl)acetamide;
N-((5-chloro-6-(isoxazol-5-ylmethoxy)-1H-indol-2-yl)methyl)acetamide;
N-((5-fluoro-6-(isoxazol-5-ylmethoxy)-1H-indol-2-yl)methyl)acetamide;
N-((5-chloro-6-((2-methyl-2H-1,2,3-triazol-4-yl)methoxy)-1H-indol-2-yl)methyl)acetamide;
N-((5-fluoro-6-((2-methyl-2H-1,2,3-triazol-4-yl)methoxy)-1H-indol-2-yl)methyl)acetamide;
N-((5-fluoro-6-((3-methylisoxazol-5-yl)methoxy)-1H-indol-2-yl)methyl)acetamide;
N-((5-fluoro-6-(oxazol-4-ylmethoxy)-1H-indol-2-yl)methyl)acetamide;
N-((5-chloro-6-((6-fluoropyridin-2-yl)methoxy)-1H-indol-2-yl)methyl)acetamide;
N-((5-fluoro-6-((6-fluoropyridin-2-yl)methoxy)-1H-indol-2-yl)methyl)acetamide;
N-((5-chloro-6-((isoxazol-3-ylmethyl)amino)-1H-indol-2-yl)methyl)acetamide;
N-((5-fluoro-6-((isoxazol-3-ylmethyl)amino)-1H-indol-2-yl)methyl)acetamide;
N-((5-chloro-6-((thiazol-4-ylmethyl)amino)-1H-indol-2-yl)methyl)acetamide;
1-fluoro-N-((5-fluoro-6-(thiazol-4-ylmethoxy)-1H-indol-2-yl)methyl)cyclopropane-1-carboxamide;
N-((5-chloro-6-(thiazol-4-ylmethoxy)-1H-indol-2-yl)methyl)-1-fluorocyclopropane-1-carboxamide;
N-((5-chloro-6-(isoxazol-3-ylmethoxy)-1H-indol-2-yl)methyl)-1-fluorocyclopropane-1-carboxamide;
1-fluoro-N-((5-fluoro-6-(isoxazol-3-ylmethoxy)-1H-indol-2-yl)methyl)cyclopropane-1-carboxamide;
N-((5-methyl-6-(thiazol-4-ylmethoxy)-1H-indol-2-yl)methyl)acetamide;
N-((6-(isoxazol-3-ylmethoxy)-5-methyl-1H-indol-2-yl)methyl)acetamide; and
N-((5-fluoro-6-((2-methyloxazol-4-yl)methoxy)-1H-indol-2-yl)methyl)acetamide;
N-((5-chloro-6-((3-methylisoxazol-5-yl)methoxy)-1H-indol-2-yl)methyl)-2-(1-hydroxycyclopropyl)acetamide;
N-((5-chloro-6-((3-methylisoxazol-5-yl)methoxy)-1H-indol-2-yl)methyl)-2-cyclopropyl-2-hydroxyacetamide;
(S)—N-((5-fluoro-6-((3-methylisoxazol-5-yl)methoxy)-1H-indol-2-yl)methyl)-2-hydroxypropanamide;
(S)—N-((5-chloro-6-((3-methylisoxazol-5-yl)methoxy)-1H-indol-2-yl)methyl)-2-hydroxybutanamide;
(R)—N-((5-chloro-6-((3-methylisoxazol-5-yl)methoxy)-1H-indol-2-yl)methyl)-2-hydroxybutanamide;
N-((5-chloro-6-((3-methylisoxazol-5-yl)methoxy)-1H-indol-2-yl)methyl)-2-hydroxyacetamide;
N-((5-chloro-6-(thiazol-4-ylmethoxy)-1H-indol-2-yl)methyl)-2-hydroxypropanamide;
N-((5-chloro-6-(thiazol-4-ylmethoxy)-1H-indol-2-yl)methyl)-2-hydroxyacetamide;
N-((5-chloro-6-((3-methylisoxazol-5-yl)methoxy)-1H-indol-2-yl)methyl)-2-(oxetan-2-yl)acetamide;
(S)—N-((5-chloro-6-((3-methylisoxazol-5-yl)methoxy)-1H-indol-2-yl)methyl)-3-fluoro-2-hydroxypropanamide;
(R)—N-((5-chloro-6-((3-methylisoxazol-5-yl)methoxy)-1H-indol-2-yl)methyl)-3-fluoro-2-hydroxypropanamide;
(1R,2R)—N-((5-chloro-6-((3-methylisoxazol-5-yl)methoxy)-1H-indol-2-yl)methyl)-2-hydroxycyclobutane-1-carboxamide;
(1S,2S)—N-((5-chloro-6-((3-methylisoxazol-5-yl)methoxy)-1H-indol-2-yl)methyl)-2-hydroxycyclobutane-1-carboxamide
N-((5-chloro-6-((3-methylisoxazol-5-yl)methoxy)-1H-indol-2-yl)methyl)-2-hydroxy-2-methylpropanamide;
N-((5-chloro-6-((3-methylisoxazol-5-yl)methoxy)-1H-indol-2-yl)methyl)-1-(hydroxymethyl)cyclopropane-1-carboxamide;
N-((5-chloro-6-((3-methylisoxazol-5-yl)methoxy)-1H-indol-2-yl)methyl)-1-hydroxycyclopropane-1-carboxamide;
(1R,3R)—N-((5-chloro-6-((3-methylisoxazol-5-yl)methoxy)-1H-indol-2-yl)methyl)-3-hydroxycyclobutane-1-carboxamide;
(1S,3S)—N-((5-chloro-6-((3-methylisoxazol-5-yl)methoxy)-1H-indol-2-yl)methyl)-3-hydroxycyclobutane-1-carboxamide;
N-((5-chloro-6-(thiazol-4-ylmethoxy)-1H-indol-2-yl)methyl)-1-(hydroxymethyl)cyclopropane-1-carboxamide;
N-((5-fluoro-6-(thiazol-4-ylmethoxy)-1H-indol-2-yl)methyl)-1-(hydroxymethyl)cyclopropane-1-carboxamide;
(R)—N-((5-chloro-6-((3-methylisoxazol-5-yl)methoxy)-1H-indol-2-yl)methyl)-2-hydroxypropanamide;
(R)—N-((5-chloro-6-(thiazol-4-ylmethoxy)-1H-indol-2-yl)methyl)-2-hydroxypropanamide;
N-((5-chloro-6-((3-methylisoxazol-5-yl)methoxy)-1H-indol-2-yl)methyl)-2-(oxetan-3-yl)acetamide;
(R)—N-((5-chloro-6-(thiazol-4-ylmethoxy)-1H-indol-2-yl)methyl)oxetane-2-carboxamide;
N-((5-chloro-6-(thiazol-4-ylmethoxy)-1H-indol-2-yl)methyl)-2-methyloxetane-2-carboxamide;
N-((5-chloro-6-(thiazol-4-ylmethoxy)-1H-indol-2-yl)methyl)oxetane-3-carboxamide;
N-((5-fluoro-6-((3-methylisoxazol-5-yl)methoxy)-1H-indol-2-yl)methyl)oxetane-2-carboxamide;
N-((5-fluoro-6-((3-methylisoxazol-5-yl)methoxy)-1H-indol-2-yl)methyl)-2-methoxyacetamide;
N-((5-fluoro-6-(thiazol-4-ylmethoxy)-1H-indol-2-yl)methyl)oxetane-2-carboxamide;
N-((5-fluoro-6-(thiazol-4-ylmethoxy)-1H-indol-2-yl)methyl)-2-methoxyacetamide;
(R)—N-((5-chloro-6-(thiazol-4-ylmethoxy)-1H-indol-2-yl)methyl)tetrahydrofuran-2-carboxamide;
N-((5-chloro-6-(thiazol-4-ylmethoxy)-1H-indol-2-yl)methyl)tetrahydrofuran-3-carboxamide;
N-((5-chloro-6-(thiazol-4-ylmethoxy)-1H-indol-2-yl)methyl)tetrahydrofuran-2-carboxamide;
N-((5-chloro-6-(thiazol-4-ylmethoxy)-1H-indol-2-yl)methyl)oxetane-2-carboxamide;
N-((5-chloro-6-(thiazol-4-ylmethoxy)-1H-indol-2-yl)methyl)-2-methoxyacetamide;
N-((5-chloro-6-(thiazol-4-ylmethoxy)-1H-indol-2-yl)methyl)-2-methoxypropanamide;
N-((5-chloro-6-((3-methylisoxazol-5-yl)methoxy)-1H-indol-2-yl)methyl)-2-methoxyacetamide;
N-((5-chloro-6-((3-methylisoxazol-5-yl)methoxy)-1H-indol-2-yl)methyl)-3-methyloxetane-3-carboxamide;
N-((5-chloro-6-((3-methylisoxazol-5-yl)methoxy)-1H-indol-2-yl)methyl)oxetane-3-carboxamide;
N-((5-chloro-6-((3-methylisoxazol-5-yl)methoxy)-1H-indol-2-yl)methyl)-3-cyanooxetane-3-carboxamide;
(S)—N-((5-chloro-6-((3-methylisoxazol-5-yl)methoxy)-1H-indol-2-yl)methyl)oxetane-2-carboxamide;
N-((5-chloro-6-(thiazol-4-ylmethoxy)-1H-indol-2-yl)methyl)-3-methyloxetane-3-carboxamide;
N-((5-chloro-6-((3-methylisoxazol-5-yl)methoxy)-1H-indol-2-yl)methyl)oxetane-2-carboxamide;
N-((5-chloro-6-(thiazol-4-ylmethoxy)-1H-indol-2-yl)methyl)-1-(methoxymethyl)cyclopropane-1-carboxamide;
N-((5-fluoro-6-(thiazol-4-ylmethoxy)-1H-indol-2-yl)methyl)-1-(methoxymethyl)cyclopropane-1-carboxamide;
(S)—N-((5-fluoro-6-(thiazol-4-ylmethoxy)-1H-indol-2-yl)methyl)-2-hydroxypropanamide;
(R)—N-((5-fluoro-6-(thiazol-4-ylmethoxy)-1H-indol-2-yl)methyl)-2-hydroxypropanamide;
(R)—N-((5-fluoro-6-((3-methylisoxazol-5-yl)methoxy)-1H-indol-2-yl)methyl)-2-hydroxypropanamide;
(S)—N-((5-chloro-6-((3-methylisoxazol-5-yl)methoxy)-1H-indol-2-yl)methyl)-2-hydroxypropanamide;
(S)—N-((5-chloro-6-(thiazol-4-ylmethoxy)-1H-indol-2-yl)methyl)-2-hydroxypropanamide;
(S)—N-((5-chloro-6-((3-methylisoxazol-5-yl)methoxy)-1H-indol-2-yl)methyl)-3-hydroxybutanamide;
(R)—N-((5-chloro-6-((3-methylisoxazol-5-yl)methoxy)-1H-indol-2-yl)methyl)-3-hydroxybutanamide;
N-((5-chloro-6-((3-methylisoxazol-5-yl)methoxy)-1H-indol-2-yl)methyl)-2-(3-methyloxetan-3-yl)acetamide;
(S)—N-((5-chloro-6-((3-methylisoxazol-5-yl)methoxy)-1H-indol-2-yl)methyl)-2-(tetrahydrofuran-3-yl)acetamide;
(R)—N-((5-chloro-6-((3-methylisoxazol-5-yl)methoxy)-1H-indol-2-yl)methyl)-2-(tetrahydrofuran-3-yl)acetamide;
(S)—N-((5-chloro-6-(thiazol-4-ylmethoxy)-1H-indol-2-yl)methyl)-2-methoxypropanamide;
(R)—N-((5-chloro-6-(thiazol-4-ylmethoxy)-1H-indol-2-yl)methyl)-2-methoxypropanamide;
(S)—N-((5-chloro-6-(thiazol-4-ylmethoxy)-1H-indol-2-yl)methyl)tetrahydrofuran-2-carboxamide;
N-((5-chloro-6-((3-methylisoxazol-5-yl)methoxy)-1H-indol-2-yl)methyl)-2-(difluoromethoxy)acetamide;
N-((5-chloro-6-((3-methylisoxazol-5-yl)methoxy)-1H-indol-2-yl)methyl)-2-(hydroxymethyl)tetrahydrofuran-2-carboxamide;
N-((5-chloro-6-((3-methylisoxazol-5-yl)methoxy)-1H-indol-2-yl)methyl)-7-oxabicyclo[2.2.1]heptane-1-carboxamide;
(1R,2S,4S)—N-((5-chloro-6-((3-methylisoxazol-5-yl)methoxy)-1H-indol-2-yl)methyl)-7-oxabicyclo[2.2.1]heptane-2-carboxamide;
(1S,2R,4R)—N-((5-chloro-6-((3-methylisoxazol-5-yl)methoxy)-1H-indol-2-yl)methyl)-7-oxabicyclo[2.2.1]heptane-2-carboxamide;
N-((5-chloro-6-((3-methylisoxazol-5-yl)methoxy)-1H-indol-2-yl)methyl)-2-oxabicyclo[2.1.1]hexane-4-carboxamide;
N-((5-chloro-6-((3-methylisoxazol-5-yl)methoxy)-1H-indol-2-yl)methyl)-2-(2-methoxyethoxy)acetamide;
N-((5-chloro-6-((3-methylisoxazol-5-yl)methoxy)-1H-indol-2-yl)methyl)-3-oxabicyclo[3.1.0]hexane-1-carboxamide;
N-((5-chloro-6-((3-methylisoxazol-5-yl)methoxy)-1H-indol-2-yl)methyl)-2-oxabicyclo[2.1.1]hexane-1-carboxamide;
(R)—N-((5-chloro-6-((3-methylisoxazol-5-yl)methoxy)-1H-indol-2-yl)methyl)-4-methylmorpholine-3-carboxamide;
(S)—N-((5-chloro-6-((3-methylisoxazol-5-yl)methoxy)-1H-indol-2-yl)methyl)-1,4-dioxane-2-carboxamide;
(R)—N-((5-chloro-6-((3-methylisoxazol-5-yl)methoxy)-1H-indol-2-yl)methyl)-1,4-dioxane-2-carboxamide;
N-((5-chloro-6-((3-methylisoxazol-5-yl)methoxy)-1H-indol-2-yl)methyl)-3-fluorotetrahydrofuran-3-carboxamide;
N-((5-chloro-6-((3-methylisoxazol-5-yl)methoxy)-1H-indol-2-yl)methyl)-3,3-difluorotetrahydrofuran-2-carboxamide;
(R)—N-((5-chloro-6-((3-methylisoxazol-5-yl)methoxy)-1H-indol-2-yl)methyl)-2-methyltetrahydrofuran-2-carboxamide;
(S)—N-((5-chloro-6-(thiazol-4-ylmethoxy)-1H-indol-2-yl)methyl)tetrahydrofuran-3-carboxamide;
N-((5-chloro-6-(thiazol-4-ylmethoxy)-1H-indol-2-yl)methyl)-3-hydroxypropanamide;
N-((5-chloro-6-((3-methylisoxazol-5-yl)methoxy)-1H-indol-2-yl)methyl)-2-(tetrahydrofuran-2-yl)acetamide;
(R)—N-((5-chloro-6-((3-methylisoxazol-5-yl)methoxy)-1H-indol-2-yl)methyl)tetrahydrofuran-2-carboxamide;
(S)—N-((5-chloro-6-((3-methylisoxazol-5-yl)methoxy)-1H-indol-2-yl)methyl)tetrahydrofuran-2-carboxamide;
(S)—N-((5-chloro-6-((3-methylisoxazol-5-yl)methoxy)-1H-indol-2-yl)methyl)-2-methoxypropanamide;
(R)—N-((5-chloro-6-((3-methylisoxazol-5-yl)methoxy)-1H-indol-2-yl)methyl)-2-methoxypropanamide;
(S)—N-((5-chloro-6-(thiazol-4-ylmethoxy)-1H-indol-2-yl)methyl)oxetane-2-carboxamide;
(R)—N-((5-chloro-6-(thiazol-4-ylmethoxy)-1H-indol-2-yl)methyl)tetrahydrofuran-3-carboxamide;
N-((5-chloro-6-((5-methyl-1,2,4-oxadiazol-3-yl)methoxy)-1H-indol-2-yl)methyl)-1-methylcyclopropane-1-carboxamide;
N-((6-((1,2,4-oxadiazol-3-yl)methoxy)-5-chloro-1H-indol-2-yl)methyl)-1-methylcyclopropane-1-carboxamide;
N-((5-chloro-6-((5-chlorothiophen-2-yl)methoxy)-1H-indol-2-yl)methyl)-1-methylcyclopropane-1-carboxamide;
N-((5-chloro-6-((2-chlorothiophen-3-yl)methoxy)-1H-indol-2-yl)methyl)-1-methylcyclopropane-1-carboxamide;
N-((5-chloro-6-(isothiazol-5-ylmethoxy)-1H-indol-2-yl)methyl)-1-methylcyclopropane-1-carboxamide;
N-((5-chloro-6-((5-chlorothiazol-2-yl)methoxy)-1H-indol-2-yl)methyl)-1-methylcyclopropane-1-carboxamide;
N-((5-chloro-6-((5-chlorothiophen-3-yl)methoxy)-1H-indol-2-yl)methyl)-1-methylcyclopropane-1-carboxamide;
N-((5-chloro-6-((4-chloroisothiazol-5-yl)methoxy)-1H-indol-2-yl)methyl)-1-methylcyclopropane-1-carboxamide;
N-((5-chloro-6-(isoxazol-5-ylmethoxy)-1H-indol-2-yl)methyl)-1-methylcyclopropane-1-carboxamide;
N-((5-fluoro-6-((6-fluoropyridin-2-yl)methoxy)-1H-indol-2-yl)methyl)-1-methylcyclopropane-1-carboxamide;
N-((5-fluoro-6-(isoxazol-5-ylmethoxy)-1H-indol-2-yl)methyl)-1-methylcyclopropane-1-carboxamide;
N-((5-fluoro-6-((5-methylisoxazol-3-yl)methoxy)-1H-indol-2-yl)methyl)-1-methylcyclopropane-1-carboxamide;
N-((5-fluoro-6-(pyridin-2-ylmethoxy)-1H-indol-2-yl)methyl)-1-methylcyclopropane-1-carboxamide;
N-((5-chloro-6-(2-(pyridin-3-yl)ethoxy)-1H-indol-2-yl)methyl)-1-methylcyclopropane-1-carboxamide;
N-((5-fluoro-6-((2-fluoropyridin-3-yl)methoxy)-1H-indol-2-yl)methyl)-1-methylcyclopropane-1-carboxamide;
N-((5-chloro-6-(2-methoxyethoxy)-1H-indol-2-yl)methyl)-1-methylcyclopropane-1-carboxamide;
N-((5-chloro-6-(pyridazin-3-ylmethoxy)-1H-indol-2-yl)methyl)-1-methylcyclopropane-1-carboxamide;
N-((5-chloro-6-(2-cyclopropoxyethoxy)-1H-indol-2-yl)methyl)-1-methylcyclopropane-1-carboxamide;
N-((5-chloro-6-(oxetan-3-ylmethoxy)-1H-indol-2-yl)methyl)-1-methylcyclopropane-1-carboxamide;
N-((5-chloro-6-((6-methoxypyridazin-3-yl)methoxy)-1H-indol-2-yl)methyl)-1-methylcyclopropane-1-carboxamide;
N-((5-chloro-6-(2-(trifluoromethoxy)ethoxy)-1H-indol-2-yl)methyl)acetamide;
N-((4-fluoro-6-((3-methylisoxazol-5-yl)methoxy)-1H-indol-2-yl)methyl)-1-methylcyclopropane-1-carboxamide;
N-((4-fluoro-6-((5-methylisoxazol-3-yl)methoxy)-1H-indol-2-yl)methyl)-1-methylcyclopropane-1-carboxamide;
N-((5-cyclopropyl-6-(thiazol-4-ylmethoxy)-1H-indol-2-yl)methyl)-1-methylcyclopropane-1-carboxamide;
N-((5-cyclopropyl-6-(isoxazol-3-ylmethoxy)-1H-indol-2-yl)methyl)-1-methylcyclopropane-1-carboxamide;
N-((5,7-dichloro-6-((5-methylisoxazol-3-yl)methoxy)-1H-indol-2-yl)methyl)-1-methylcyclopropane-1-carboxamide;
N-((5,7-dichloro-6-(thiazol-4-ylmethoxy)-1H-indol-2-yl)methyl)-1-methylcyclopropane-1-carboxamide;
N-((5-chloro-6-((3-methylisoxazol-5-yl)methoxy)-1H-indol-2-yl)methyl-d2)acetamide;
N-((5,7-dichloro-6-(isoxazol-3-ylmethoxy)-1H-indol-2-yl)methyl)-1-methylcyclopropane-1-carboxamide;
(S)—N-(1-(5-chloro-6-(thiazol-4-ylmethoxy)-1H-indol-2-yl)ethyl)-1-methylcyclopropane-1-carboxamide;
(R)—N-(1-(5-chloro-6-(thiazol-4-ylmethoxy)-1H-indol-2-yl)ethyl)-1-methylcyclopropane-1-carboxamide;
1-methyl-N-((5-(methylthio)-6-(thiazol-4-ylmethoxy)-1H-indol-2-yl)methyl)cyclopropane-1-carboxamide;
N-((6-((3-methylisoxazol-5-yl)methoxy)-1H-indol-2-yl)methyl)acetamide;
N-((6-((1H-indol-6-yl)methoxy)-5-chloro-1H-indol-2-yl)methyl)-1-methylcyclopropane-1-carboxamide;
N-((5-chloro-6-((2-chloro-4-fluoropyridin-3-yl)methoxy)-1H-indol-2-yl)methyl)-1-methylcyclopropane-1-carboxamide;
N-((5-chloro-6-((5-(trifluoromethyl)isoxazol-3-yl)methoxy)-1H-indol-2-yl)methyl)-1-methylcyclopropane-1-carboxamide;
N-((5-chloro-6-((3,3-difluorocyclobutyl)methoxy)-1H-indol-2-yl)methyl)-1-methylcyclopropane-1-carboxamide;
1-methyl-N-((6-((5-(trifluoromethyl)isoxazol-3-yl)methoxy)-1H-indol-2-yl)methyl)cyclopropane-1-carboxamide;
N-((5-chloro-6-(oxetan-3-ylmethoxy)-1H-indol-2-yl)methyl)acetamide;
N-((5-chloro-6-((4-fluoropyridin-3-yl)methoxy)-1H-indol-2-yl)methyl)-1-methylcyclopropane-1-carboxamide;
N-((5-fluoro-6-(thiazol-4-ylmethoxy)-1H-indol-2-yl)methyl)-3-methylazetidine-1-carboxamide;
N-((5-fluoro-6-(oxazol-4-ylmethoxy)-1H-indol-2-yl)methyl)azetidine-1-carboxamide;
3-((5-chloro-6-(thiazol-4-ylmethoxy)-1H-indol-2-yl)methyl)-1-cyclopropyl-1-methylurea;
(S)—N-((5-chloro-6-(thiazol-4-ylmethoxy)-1H-indol-2-yl)methyl)-2-methylazetidine-1-carboxamide;
(R)—N-((5-chloro-6-(thiazol-4-ylmethoxy)-1H-indol-2-yl)methyl)-2-methylazetidine-1-carboxamide;
(S)—N-((5-chloro-6-(isoxazol-3-ylmethoxy)-1H-indol-2-yl)methyl)-2-methylazetidine-1-carboxamide;
(R)—N-((5-chloro-6-(isoxazol-3-ylmethoxy)-1H-indol-2-yl)methyl)-2-methylazetidine-1-carboxamide;
N-((5-chloro-6-(thiazol-4-ylmethoxy)-1H-indol-2-yl)methyl)-3-methoxyazetidine-1-carboxamide;
(S)—N-((5-chloro-6-(thiazol-4-ylmethoxy)-1H-indol-2-yl)methyl)-2-(hydroxymethyl)azetidine-1-carboxamide;
(R)—N-((5-chloro-6-(thiazol-4-ylmethoxy)-1H-indol-2-yl)methyl)-2-(hydroxymethyl)azetidine-1-carboxamide;
(R)—N-((5-chloro-6-(thiazol-4-ylmethoxy)-1H-indol-2-yl)methyl)-3-hydroxypyrrolidine-1-carboxamide;
(S)—N-((5-chloro-6-(thiazol-4-ylmethoxy)-1H-indol-2-yl)methyl)-3-hydroxypyrrolidine-1-carboxamide;
N-((5-chloro-6-(thiazol-4-ylmethoxy)-1H-indol-2-yl)methyl)-3-hydroxyazetidine-1-carboxamide;
N-((5-chloro-6-(thiazol-4-ylmethoxy)-1H-indol-2-yl)methyl)-3-(hydroxymethyl)azetidine-1-carboxamide;
1-((5-chloro-6-((3-methylisoxazol-5-yl)methoxy)-1H-indol-2-yl)methyl)-3-ethylurea;
N-((5-chloro-6-((3-methylisoxazol-5-yl)methoxy)-1H-indol-2-yl)methyl)-3,3-difluoroazetidine-1-carboxamide;
1-((5-chloro-6-((3-methylisoxazol-5-yl)methoxy)-1H-indol-2-yl)methyl)-3-(2-fluoroethyl)urea;
N-((5-chloro-6-((3-methylisoxazol-5-yl)methoxy)-1H-indol-2-yl)methyl)-3-fluoroazetidine-1-carboxamide; (R)—N-((5-chloro-6-((3-methylisoxazol-5-yl)methoxy)-1H-indol-2-yl)methyl)-3-fluoropyrrolidine-1-carboxamide; (S)—N-((5-chloro-6-((3-methylisoxazol-5-yl)methoxy)-1H-indol-2-yl)methyl)-3-fluoropyrrolidine-1-carboxamide;
1-((5-chloro-6-((3-methylisoxazol-5-yl)methoxy)-1H-indol-2-yl)methyl)-3-(2,2-difluoroethyl)urea;
(S)—N-((5-chloro-6-((3-methylisoxazol-5-yl)methoxy)-1H-indol-2-yl)methyl)-2-(difluoromethyl)pyrrolidine-1-carboxamide;
(R)—N-((5-chloro-6-((3-methylisoxazol-5-yl)methoxy)-1H-indol-2-yl)methyl)-2-(difluoromethyl)pyrrolidine-1-carboxamide;
N-((5-chloro-6-((3-methylisoxazol-5-yl)methoxy)-1H-indol-2-yl)methyl)-2-cyanoazetidine-1-carboxamide;
N-((5-chloro-6-((3-methylisoxazol-5-yl)methoxy)-1H-indol-2-yl)methyl)-3-(difluoromethyl)pyrrolidine-1-carboxamide;
N-((5-chloro-6-((3-methylisoxazol-5-yl)methoxy)-1H-indol-2-yl)methyl)-6-oxa-1-azaspiro[3.3]heptane-1-carboxamide;
3-((5-chloro-6-(isoxazol-3-ylmethoxy)-1H-indol-2-yl)methyl)-1-cyclopropyl-1-methylurea;
1-((5-chloro-6-(thiazol-4-ylmethoxy)-1H-indol-2-yl)methyl)-3-(2-methoxyethyl)urea;
(R)—N-((5-fluoro-6-(isoxazol-3-ylmethoxy)-1H-indol-2-yl)methyl)-3-methylpyrrolidine-1-carboxamide;
(S)—N-((5-fluoro-6-(isoxazol-3-ylmethoxy)-1H-indol-2-yl)methyl)-3-methylpyrrolidine-1-carboxamide;
(R)—N-((5-fluoro-6-(isoxazol-3-ylmethoxy)-1H-indol-2-yl)methyl)-2-methylpyrrolidine-1-carboxamide;
(S)—N-((5-fluoro-6-(isoxazol-3-ylmethoxy)-1H-indol-2-yl)methyl)-2-methylpyrrolidine-1-carboxamide;
(S)—N-((5-fluoro-6-(thiazol-4-ylmethoxy)-1H-indol-2-yl)methyl)-2-methylpyrrolidine-1-carboxamide;
(R)—N-((5-fluoro-6-(thiazol-4-ylmethoxy)-1H-indol-2-yl)methyl)-2-methylpyrrolidine-1-carboxamide;
(R)—N-((5-fluoro-6-(thiazol-4-ylmethoxy)-1H-indol-2-yl)methyl)-3-methylpyrrolidine-1-carboxamide;
(S)—N-((5-fluoro-6-(thiazol-4-ylmethoxy)-1H-indol-2-yl)methyl)-3-methylpyrrolidine-1-carboxamide;
N-((5-fluoro-6-(isoxazol-3-ylmethoxy)-1H-indol-2-yl)methyl)-3-methylazetidine-1-carboxamide;
N-((5-chloro-6-(isoxazol-3-ylmethoxy)-1H-indol-2-yl)methyl)-3-methylazetidine-1-carboxamide;
N-((6-(isoxazol-3-ylmethoxy)-5-(methylthio)-1H-indol-2-yl)methyl)-1-methylcyclopropane-1-carboxamide;
N-((5-chloro-6-(pyrazolo [1,5-a]pyridin-2-ylmethoxy)-1H-indol-2-yl)methyl)-1-methylcyclopropane-1-carboxamide;
N-((5-chloro-6-(2-(3,3-difluorocyclobutyl)ethoxy)-1H-indol-2-yl)methyl)-1-methylcyclopropane-1-carboxamide;
N-((5-chloro-6-(2-cyclobutylethoxy)-1H-indol-2-yl)methyl)-1-methylcyclopropane-1-carboxamide;
(S)—N-(1-(5-chloro-6-(isoxazol-3-ylmethoxy)-1H-indol-2-yl)ethyl)-1-methylcyclopropane-1-carboxamide;
(R)—N-(1-(5-chloro-6-(isoxazol-3-ylmethoxy)-1H-indol-2-yl)ethyl)-1-methylcyclopropane-1-carboxamide;
(S)—N-((6-((1,4-dioxan-2-yl)methoxy)-5-chloro-1H-indol-2-yl)methyl)-1-methylcyclopropane-1-carboxamide;
(R)—N-((6-((1,4-dioxan-2-yl)methoxy)-5-chloro-1H-indol-2-yl)methyl)-1-methylcyclopropane-1-carboxamide;
(S)—N-((5-chloro-6-(2-(tetrahydrofuran-2-yl)ethoxy)-1H-indol-2-yl)methyl)-1-methylcyclopropane-1-carboxamide;
(R)—N-((5-chloro-6-(2-(tetrahydrofuran-2-yl)ethoxy)-1H-indol-2-yl)methyl)-1-methylcyclopropane-1-carboxamide;
N-((5-chloro-6-((3-methylisothiazol-5-yl)methoxy)-1H-indol-2-yl)methyl)-1-methylcyclopropane-1-carboxamide;
N-((5-chloro-6-((5-methylisothiazol-3-yl)methoxy)-1H-indol-2-yl)methyl)-1-methylcyclopropane-1-carboxamide;
N-((5-chloro-6-((5-chlorothiazol-4-yl)methoxy)-1H-indol-2-yl)methyl)-1-methylcyclopropane-1-carboxamide;
N-((5-fluoro-6-(isoxazol-5-ylmethoxy)-1H-indol-2-yl)methyl)azetidine-1-carboxamide;
N-((5-chloro-6-((6-fluoropyridin-2-yl)methoxy)-1H-indol-2-yl)methyl)azetidine-1-carboxamide;
N-((5-chloro-6-(oxazol-4-ylmethoxy)-1H-indol-2-yl)methyl)azetidine-1-carboxamide;
N-((5-chloro-6-((3-methylisoxazol-5-yl)methoxy)-1H-indol-2-yl)methyl)azetidine-1-carboxamide;
N-((5-chloro-6-((2-methyl-2H-1,2,3-triazol-4-yl)methoxy)-1H-indol-2-yl)methyl)azetidine-1-carboxamide;
N-((5-chloro-6-((2-methyl-2H-1,2,3-triazol-4-yl)methoxy)-1H-indol-2-yl)methyl)pyrrolidine-1-carboxamide;
N-((5-chloro-6-((isoxazol-3-ylmethyl)amino)-1H-indol-2-yl)methyl)azetidine-1-carboxamide;
N-((5-chloro-6-((2-methyloxazol-4-yl)methoxy)-1H-indol-2-yl)methyl)azetidine-1-carboxamide;
N-((5-fluoro-6-((2-methyl-2H-1,2,3-triazol-4-yl)methoxy)-1H-indol-2-yl)methyl)azetidine-1-carboxamide;
N-((5-fluoro-6-((2-methyl-2H-1,2,3-triazol-4-yl)methoxy)-1H-indol-2-yl)methyl)pyrrolidine-1-carboxamide;
N-((5-chloro-6-((2-methyloxazol-4-yl)methoxy)-1H-indol-2-yl)methyl)pyrrolidine-1-carboxamide;
N-((5-chloro-6-(2-(isoxazol-3-yl)ethyl)-1H-indol-2-yl)methyl)pyrrolidine-1-carboxamide;
N-((5-chloro-6-((isoxazol-3-ylmethyl)amino)-1H-indol-2-yl)methyl)pyrrolidine-1-carboxamide;
N-((5-chloro-6-((thiazol-4-ylmethyl)amino)-1H-indol-2-yl)methyl)pyrrolidine-1-carboxamide;
N-((6-(thiazol-4-ylmethoxy)-5-(trifluoromethoxy)-1H-indol-2-yl)methyl)pyrrolidine-1-carboxamide;
N-((6-(isoxazol-3-ylmethoxy)-5-(trifluoromethoxy)-1H-indol-2-yl)methyl)azetidine-1-carboxamide;
N-((6-(oxazol-4-ylmethoxy)-5-(trifluoromethoxy)-1H-indol-2-yl)methyl)pyrrolidine-1-carboxamide;
N-((5-chloro-6-(2-(thiazol-4-yl)ethyl)-1H-indol-2-yl)methyl)pyrrolidine-1-carboxamide;
N-((6-(2-(thiazol-4-yl)ethyl)-5-(trifluoromethoxy)-1H-indol-2-yl)methyl)pyrrolidine-1-carboxamide;
N-((6-(isoxazol-3-ylmethoxy)-5-(trifluoromethoxy)-1H-indol-2-yl)methyl)pyrrolidine-1-carboxamide;
N-((6-(thiazol-4-ylmethoxy)-5-(trifluoromethoxy)-1H-indol-2-yl)methyl)azetidine-1-carboxamide;
N-((4-fluoro-6-(thiazol-4-ylmethoxy)-1H-indol-2-yl)methyl)azetidine-1-carboxamide;
N-((4-fluoro-6-(isoxazol-3-ylmethoxy)-1H-indol-2-yl)methyl)azetidine-1-carboxamide;
N-((5-chloro-3-ethyl-6-(thiazol-4-ylmethoxy)-1H-indol-2-yl)methyl)azetidine-1-carboxamide;
N-((5-chloro-6-((6-fluoropyridin-2-yl)methoxy)-1H-indol-2-yl)methyl)pyrrolidine-1-carboxamide;
N-((5-chloro-6-((4-methyloxazol-2-yl)methoxy)-1H-indol-2-yl)methyl)pyrrolidine-1-carboxamide;
N-((4-fluoro-6-((3-methylisoxazol-5-yl)methoxy)-1H-indol-2-yl)methyl)azetidine-1-carboxamide;
N-((5-fluoro-6-(oxazol-4-ylmethoxy)-1H-indol-2-yl)methyl)cyclopropanecarboxamide;
N-((5-chloro-6-(oxazol-4-ylmethoxy)-1H-indol-2-yl)methyl)cyclopropanecarboxamide;
N-((5-fluoro-6-(thiazol-4-ylmethoxy)-1H-indol-2-yl)methyl)cyclobutanecarboxamide;
N-((5-fluoro-6-(isoxazol-3-ylmethoxy)-1H-indol-2-yl)methyl)cyclobutanecarboxamide;
N-((5-chloro-6-(thiazol-4-ylmethoxy)-1H-indol-2-yl)methyl)pivalamide;
N-((5-fluoro-6-((2-methyl-2H-1,2,3-triazol-4-yl)methoxy)-1H-indol-2-yl)methyl)cyclopropanecarboxamide;
1-methyl-N-((6-(thiazol-4-ylmethoxy)-5-(trifluoromethoxy)-1H-indol-2-yl)methyl)cyclopropane-1-carboxamide;
N-((5-chloro-6-(thiazol-4-ylmethoxy)-1H-indol-2-yl)methyl)-2,2-difluoropropanamide;
2,2-difluoro-N-((5-fluoro-6-(thiazol-4-ylmethoxy)-1H-indol-2-yl)methyl)propanamide;
N-((5-chloro-6-(thiazol-4-ylmethoxy)-1H-indol-2-yl)methyl)-2-cyanoacetamide;
N-((5-chloro-6-((3-methylisoxazol-5-yl)methoxy)-1H-indol-2-yl)methyl)propionamide;
1-methyl-N-((6-(oxazol-4-ylmethoxy)-5-(trifluoromethoxy)-1H-indol-2-yl)methyl)cyclopropane-1-carboxamide;
N-((5-chloro-6-((3-methylisoxazol-5-yl)methoxy)-1H-indol-2-yl)methyl)-2,2-difluoropropanamide;
2,2-difluoro-N-((5-fluoro-6-((3-methylisoxazol-5-yl)methoxy)-1H-indol-2-yl)methyl)propanamide;
(S)—N-((5-chloro-6-((3-methylisoxazol-5-yl)methoxy)-1H-indol-2-yl)methyl)-2-fluoropropanamide;
(R)—N-((5-chloro-6-((3-methylisoxazol-5-yl)methoxy)-1H-indol-2-yl)methyl)-2-fluoropropanamide;
(S)—N-((5-chloro-6-(thiazol-4-ylmethoxy)-1H-indol-2-yl)methyl)-2-fluoropropanamide;
(R)—N-((5-chloro-6-(thiazol-4-ylmethoxy)-1H-indol-2-yl)methyl)-2-fluoropropanamide;
(S)—N-((5-chloro-6-(thiazol-4-ylmethoxy)-1H-indol-2-yl)methyl)-2-cyanopropanamide;
(R)—N-((5-chloro-6-(thiazol-4-ylmethoxy)-1H-indol-2-yl)methyl)-2-cyanopropanamide;
(R)—N-((5-chloro-6-((3-methylisoxazol-5-yl)methoxy)-1H-indol-2-yl)methyl)-2-cyanopropanamide;
(S)—N-((5-chloro-6-((3-methylisoxazol-5-yl)methoxy)-1H-indol-2-yl)methyl)-2-cyanopropanamide;
(S)—N-((5-chloro-6-((3-methylisoxazol-5-yl)methoxy)-1H-indol-2-yl)methyl)azetidine-2-carboxamide;
N-((5-chloro-6-((3-methylisoxazol-5-yl)methoxy)-1H-indol-2-yl)methyl)-2-cyclopropylacetamide;
N-((5-chloro-6-((3-methylisoxazol-5-yl)methoxy)-1H-indol-2-yl)methyl)isoxazole-3-carboxamide;
N-((5-chloro-6-(((5-methylisoxazol-3-yl)methyl)amino)-1H-indol-2-yl)methyl)-1-methylcyclopropane-1-carboxamide;
N-((5-chloro-6-((thiazol-4-ylmethyl)amino)-1H-indol-2-yl)methyl)-1-methylcyclopropane-1-carboxamide;
N-((4-fluoro-6-(isoxazol-3-ylmethoxy)-1H-indol-2-yl)methyl)acetamide;
N-((5-chloro-6-(2-methoxypropoxy)-1H-indol-2-yl)methyl)-1-methylcyclopropane-1-carboxamide;
N-((5-chloro-6-(2-(tetrahydrofuran-2-yl)ethoxy)-1H-indol-2-yl)methyl)acetamide;
N-((5-chloro-6-(2-cyclobutoxyethoxy)-1H-indol-2-yl)methyl)acetamide;
N-((6-((1,4-dioxan-2-yl)methoxy)-5-chloro-1H-indol-2-yl)methyl)acetamide;
N-((5-chloro-6-((2,2-difluorocyclopropyl)methoxy)-1H-indol-2-yl)methyl)acetamide;
N-((5-chloro-6-(2-ethoxyethoxy)-1H-indol-2-yl)methyl)acetamide;
N-((6-(benzofuran-6-ylmethoxy)-5-fluoro-1H-indol-2-yl)methyl)-1-methylcyclopropane-1-carboxamide;
N-((6-(benzofuran-6-ylmethoxy)-5-chloro-1H-indol-2-yl)methyl)-1-methylcyclopropane-1-carboxamide;
N-((5-chloro-6-((trans)-3-methoxycyclobutoxy)-1H-indol-2-yl)methyl)acetamide;
N-((5-chloro-6-((cis)-3-methoxycyclobutoxy)-1H-indol-2-yl)methyl)acetamide;
(S)—N-((5-chloro-6-((tetrahydrofuran-3-yl)methoxy)-1H-indol-2-yl)methyl)acetamide;
(R)—N-((5-chloro-6-((tetrahydrofuran-2-yl)methoxy)-1H-indol-2-yl)methyl)acetamide;
(S)—N-((5-chloro-6-((tetrahydrofuran-2-yl)methoxy)-1H-indol-2-yl)methyl)acetamide;
N-((6-(2-(oxazol-4-yl)ethyl)-5-(trifluoromethoxy)-1H-indol-2-yl)methyl)azetidine-1-carboxamide;
N-((6-(2-(oxazol-4-yl)ethyl)-5-(trifluoromethoxy)-1H-indol-2-yl)methyl)pyrrolidine-1-carboxamide;
N-((5-chloro-6-(((5-methylisoxazol-3-yl)methyl)amino)-1H-indol-2-yl)methyl)pyrrolidine-1-carboxamide;
N-((5-fluoro-6-((isoxazol-3-ylmethyl)amino)-1H-indol-2-yl)methyl)azetidine-1-carboxamide;
N-((5-fluoro-6-((isoxazol-3-ylmethyl)amino)-1H-indol-2-yl)methyl)pyrrolidine-1-carboxamide;
N-((5-chloro-6-((thiazol-4-ylmethyl)amino)-1H-indol-2-yl)methyl)azetidine-1-carboxamide;
N-((5-chloro-3-methyl-6-(thiazol-4-ylmethoxy)-1H-indol-2-yl)methyl)azetidine-1-carboxamide;
N-((3,5-dichloro-6-(thiazol-4-ylmethoxy)-1H-indol-2-yl)methyl)azetidine-1-carboxamide;
N-((3-chloro-5-methyl-6-(thiazol-4-ylmethoxy)-1H-indol-2-yl)methyl)azetidine-1-carboxamide;
N-((6-(isoxazol-3-ylmethoxy)-5-(trifluoromethyl)-1H-indol-2-yl)methyl)pyrrolidine-1-carboxamide;
N-((6-(isoxazol-3-ylmethoxy)-5-(trifluoromethyl)-1H-indol-2-yl)methyl)azetidine-1-carboxamide;
N-((6-(thiazol-4-ylmethoxy)-5-(trifluoromethyl)-1H-indol-2-yl)methyl)azetidine-1-carboxamide;
N-((6-(thiazol-4-ylmethoxy)-5-(trifluoromethyl)-1H-indol-2-yl)methyl)pyrrolidine-1-carboxamide;
N-((6-(oxazol-4-ylmethoxy)-5-(trifluoromethoxy)-1H-indol-2-yl)methyl)azetidine-1-carboxamide;
N-((5-fluoro-6-((3-methylisoxazol-5-yl)methoxy)-1H-indol-2-yl)methyl)azetidine-1-carboxamide;
N-((5-fluoro-6-(thiazol-4-ylmethoxy)-1H-indol-2-yl)methyl)-1-(methyl-d3)cyclopropane-1-carboxamide;
N-((5-chloro-6-(thiazol-4-ylmethoxy)-1H-indol-2-yl)methyl)-1-(methyl-d3)cyclopropane-1-carboxamide;
N-((5-chloro-6-((3-methylisoxazol-5-yl)methoxy)-1H-indol-2-yl)methyl)-1-cyanocyclopropane-1-carboxamide;
N-((5-chloro-6-((3-methylisoxazol-5-yl)methoxy)-1H-indol-2-yl)methyl)-2-cyano-2-methylpropanamide;
(S)—N-((5-chloro-6-((3-methylisoxazol-5-yl)methoxy)-1H-indol-2-yl)methyl)-2-cyanopropanamide;
(R)—N-((5-chloro-6-((3-methylisoxazol-5-yl)methoxy)-1H-indol-2-yl)methyl)-2-cyanopropanamide;
N-((5-chloro-6-((3-methylisoxazol-5-yl)methoxy)-1H-indol-2-yl)methyl)-1-cyanocyclobutane-1-carboxamide;
N-((5-chloro-6-(isoxazol-3-ylmethoxy)-1H-indol-2-yl)methyl)cyclobutanecarboxamide;
N-((5-chloro-6-(thiazol-4-ylmethoxy)-1H-indol-2-yl)methyl)cyclopentanecarboxamide;
N-((5-chloro-6-(thiazol-4-ylmethoxy)-1H-indol-2-yl)methyl)cyclobutanecarboxamide;
N-((5-chloro-6-(isoxazol-3-ylmethoxy)-1H-indol-2-yl)methyl)-3,3-difluorocyclobutane-1-carboxamide;
N-((5-chloro-6-(isoxazol-3-ylmethoxy)-1H-indol-2-yl)methyl)cyclopentanecarboxamide;
N-((5-chloro-6-(isoxazol-3-ylmethoxy)-1H-indol-2-yl)methyl)pivalamide;
N-((5-chloro-6-((6-fluoropyridin-2-yl)methoxy)-1H-indol-2-yl)methyl)propionamide;
N-((5-chloro-6-(thiazol-4-ylmethoxy)-1H-indol-2-yl)methyl)-3,3-difluorocyclobutane-1-carboxamide;
N-((5-fluoro-6-((2-methyl-2H-1,2,3-triazol-4-yl)methoxy)-1H-indol-2-yl)methyl)-1-methylcyclopropane-1-carboxamide;
N-((5-fluoro-6-((6-fluoropyridin-2-yl)methoxy)-1H-indol-2-yl)methyl)cyclopropanecarboxamide;
N-((5-chloro-6-((2-methyl-2H-1,2,3-triazol-4-yl)methoxy)-1H-indol-2-yl)methyl)cyclopropanecarboxamide;
N-((5-chloro-6-((6-fluoropyridin-2-yl)methoxy)-1H-indol-2-yl)methyl)cyclopropanecarboxamide;
N-((5-chloro-6-(oxazol-4-ylmethoxy)-1H-indol-2-yl)methyl)isobutyramide;
N-((5-fluoro-6-(oxazol-4-ylmethoxy)-1H-indol-2-yl)methyl)isobutyramide;
N-((5-fluoro-6-(isoxazol-3-ylmethoxy)-1H-indol-2-yl)methyl)-1-methylcyclobutane-1-carboxamide;
N-((5-chloro-6-(pyridin-2-ylmethoxy)-1H-indol-2-yl)methyl)cyclopropanecarboxamide;
N-((5-chloro-6-(isoxazol-3-ylmethoxy)-1H-indol-2-yl)methyl)-1-methylcyclobutane-1-carboxamide;
N-((5-fluoro-6-(pyridin-2-ylmethoxy)-1H-indol-2-yl)methyl)cyclopropanecarboxamide;
N-((5-chloro-6-(thiazol-4-ylmethoxy)-1H-indol-2-yl)methyl)-1-methylcyclobutane-1-carboxamide;
N-((5-fluoro-6-(thiazol-4-ylmethoxy)-1H-indol-2-yl)methyl)-1-methylcyclobutane-1-carboxamide;
N-((6-(isoxazol-3-ylmethoxy)-5-(trifluoromethoxy)-1H-indol-2-yl)methyl)-1-methylcyclopropane-1-carboxamide;
1-methyl-N-((6-(2-(thiazol-4-yl)ethyl)-5-(trifluoromethoxy)-1H-indol-2-yl)methyl)cyclopropane-1-carboxamide;
N-((6-(2-(isoxazol-3-yl)ethyl)-5-(trifluoromethoxy)-1H-indol-2-yl)methyl)-1-methylcyclopropane-1-carboxamide;
(R)—N-((5-chloro-6-(thiazol-4-ylmethoxy)-1H-indol-2-yl)methyl)-2-fluoropropanamide;
(S)—N-((5-chloro-6-(thiazol-4-ylmethoxy)-1H-indol-2-yl)methyl)-2-fluoropropanamide;
2-(azetidin-1-yl)-N-((5-chloro-6-(thiazol-4-ylmethoxy)-1H-indol-2-yl)methyl)acetamide;
N-((5-chloro-6-(thiazol-4-ylmethoxy)-1H-indol-2-yl)methyl)-3,3-difluoropropanamide;
1-methyl-N-((6-(2-(oxazol-4-yl)ethyl)-5-(trifluoromethoxy)-1H-indol-2-yl)methyl)cyclopropane-1-carboxamide;
N-((4-fluoro-6-(thiazol-4-ylmethoxy)-1H-indol-2-yl)methyl)propionamide;
3,3-difluoro-N-((5-fluoro-6-(thiazol-4-ylmethoxy)-1H-indol-2-yl)methyl)propanamide;
N-((4-fluoro-6-(isoxazol-3-ylmethoxy)-1H-indol-2-yl)methyl)propionamide;
3,3-difluoro-N-((5-fluoro-6-((3-methylisoxazol-5-yl)methoxy)-1H-indol-2-yl)methyl)propanamide;
N-((5-chloro-6-((3-methylisoxazol-5-yl)methoxy)-1H-indol-2-yl)methyl)-3,3-difluoropropanamide;
N-((5-chloro-6-((3-methylisoxazol-5-yl)methoxy)-1H-indol-2-yl)methyl)-2-(1H-1,2,3-triazol-1-yl)acetamide;
N-((5-chloro-6-((3-methylisoxazol-5-yl)methoxy)-1H-indol-2-yl)methyl)-2-(1H-pyrazol-1-yl)acetamide;
N-((5-chloro-6-((3-methylisoxazol-5-yl)methoxy)-1H-indol-2-yl)methyl)isoxazole-5-carboxamide;
N-((5-fluoro-6-((thiazol-4-ylmethyl)amino)-1H-indol-2-yl)methyl)-1-methylcyclopropane-1-carboxamide;
N-((5-fluoro-6-((isoxazol-3-ylmethyl)amino)-1H-indol-2-yl)methyl)-1-methylcyclopropane-1-carboxamide;
N-((4-fluoro-6-(thiazol-4-ylmethoxy)-1H-indol-2-yl)methyl)acetamide;
N-((4,5-difluoro-6-(thiazol-4-ylmethoxy)-1H-indol-2-yl)methyl)-1-methylcyclopropane-1-carboxamide;
N-((4-fluoro-6-((3-methylisoxazol-5-yl)methoxy)-1H-indol-2-yl)methyl)acetamide;
N-((5-methyl-6-(thiazol-4-ylmethoxy)-1H-indol-2-yl)methyl)azetidine-1-carboxamide;
N-((5-methyl-6-(thiazol-4-ylmethoxy)-1H-indol-2-yl)methyl)propionamide;
N-((6-(isoxazol-3-ylmethoxy)-5-methyl-1H-indol-2-yl)methyl)propionamide;
N-((5-methyl-6-(thiazol-4-ylmethoxy)-1H-indol-2-yl)methyl)pyrrolidine-1-carboxamide;
N-((6-(isoxazol-3-ylmethoxy)-5-methyl-1H-indol-2-yl)methyl)azetidine-1-carboxamide;
N-((6-(isoxazol-3-ylmethoxy)-5-methyl-1H-indol-2-yl)methyl)pyrrolidine-1-carboxamide;
(1R,2R)—N-((5-chloro-6-((3-methylisoxazol-5-yl)methoxy)-1H-indol-2-yl)methyl)-2-hydroxycyclopentane-1-carboxamide;
(1S,2S)—N-((5-chloro-6-((3-methylisoxazol-5-yl)methoxy)-1H-indol-2-yl)methyl)-2-hydroxycyclopentane-1-carboxamide;
(S)—N-((5-chloro-6-((3-methylisoxazol-5-yl)methoxy)-1H-indol-2-yl)methyl)-3-hydroxy-2-methylpropanamide;
(R)—N-((5-chloro-6-((3-methylisoxazol-5-yl)methoxy)-1H-indol-2-yl)methyl)-3-hydroxy-2-methylpropanamide;
(1S,2R)—N-((5-chloro-6-((3-methylisoxazol-5-yl)methoxy)-1H-indol-2-yl)methyl)-2-fluorocyclopropane-1-carboxamide;
(1R,2S)—N-((5-chloro-6-((3-methylisoxazol-5-yl)methoxy)-1H-indol-2-yl)methyl)-2-fluorocyclopropane-1-carboxamide;
(1R,2R)—N-((5-chloro-6-((3-methylisoxazol-5-yl)methoxy)-1H-indol-2-yl)methyl)-2-fluorocyclopropane-1-carboxamide;
(1S,2S)—N-((5-chloro-6-((3-methylisoxazol-5-yl)methoxy)-1H-indol-2-yl)methyl)-2-fluorocyclopropane-1-carboxamide;
(1R,2R)—N-((5-chloro-6-(thiazol-4-ylmethoxy)-1H-indol-2-yl)methyl)-2-fluorocyclopropane-1-carboxamide;
(1S,2S)—N-((5-chloro-6-(thiazol-4-ylmethoxy)-1H-indol-2-yl)methyl)-2-fluorocyclopropane-1-carboxamide;
(1S,2R)—N-((5-chloro-6-(thiazol-4-ylmethoxy)-1H-indol-2-yl)methyl)-2-fluorocyclopropane-1-carboxamide;
(1R,2S)—N-((5-chloro-6-(thiazol-4-ylmethoxy)-1H-indol-2-yl)methyl)-2-fluorocyclopropane-1-carboxamide;
(S)—N-((5-chloro-6-((3-methylisoxazol-5-yl)methoxy)-1H-indol-2-yl)methyl)-2-(oxetan-3-yl)propenamide;
(R)—N-((5-chloro-6-((3-methylisoxazol-5-yl)methoxy)-1H-indol-2-yl)methyl)-2-(oxetan-3-yl)propanamide;
(R)—N-((5-chloro-6-(thiazol-4-ylmethoxy)-1H-indol-2-yl)methyl)-1-methyl-2-oxoazetidine-3-carboxamide;
(S)—N-((5-chloro-6-(thiazol-4-ylmethoxy)-1H-indol-2-yl)methyl)-1-methyl-2-oxoazetidine-3-carboxamide;
N-((5-fluoro-6-((2-methyloxazol-4-yl)methoxy)-1H-indol-2-yl)methyl)-1-methylcyclopropane-1-carboxamide;
N-((5-fluoro-6-((3-methylisoxazol-5-yl)methoxy)-1H-indol-2-yl)methyl)propionamide;
N-((5-fluoro-6-((2-methyloxazol-4-yl)methoxy)-1H-indol-2-yl)methyl)cyclopropanecarboxamide;
N-((5-fluoro-6-((2-methyloxazol-4-yl)methoxy)-1H-indol-2-yl)methyl)propionamide;
N-((5-fluoro-6-((2-methyloxazol-4-yl)methoxy)-1H-indol-2-yl)methyl)isobutyramide;
N-((5-chloro-6-(thiazol-4-ylmethoxy)-1H-indol-2-yl)methyl)bicyclo[1.1.1]pentane-1-carboxamide;
N-((5-chloro-6-(thiazol-4-ylmethoxy)-1H-indol-2-yl)methyl)spiro[2.2]pentane-1-carboxamide;
N-((5-chloro-6-(thiazol-4-ylmethoxy)-1H-indol-2-yl)methyl)-3,3,3-trifluoropropanamide;
2-(bicyclo[1.1.1]pentan-1-yl)-N-((5-chloro-6-(thiazol-4-ylmethoxy)-1H-indol-2-yl)methyl)acetamide;
N-((5-chloro-6-(thiazol-4-ylmethoxy)-1H-indol-2-yl)methyl)bicyclo[2.2.1]heptane-1-carboxamide;
N-((5-chloro-6-(thiazol-4-ylmethoxy)-1H-indol-2-yl)methyl)spiro[3.3]heptane-2-carboxamide;
N-((5-chloro-6-(thiazol-4-ylmethoxy)-1H-indol-2-yl)methyl)spiro[2.3]hexane-1-carboxamide;
N-((5-chloro-6-(thiazol-4-ylmethoxy)-1H-indol-2-yl)methyl)-1-(difluoromethyl)cyclopropane-1-carboxamide;
N-((5-chloro-6-(thiazol-4-ylmethoxy)-1H-indol-2-yl)methyl)-2,2-difluoroacetamide;
N-((5-chloro-6-(thiazol-4-ylmethoxy)-1H-indol-2-yl)methyl)-3-methylcyclobutane-1-carboxamide;
N-((5-chloro-6-((3-methylisoxazol-5-yl)methoxy)-1H-indol-2-yl)methyl)acetamide-2,2,2-d3;
N-((5-chloro-6-((3-methylisoxazol-5-yl)methoxy)-1H-indol-2-yl)methyl)-2,2-difluoroacetamide;
N-((5-chloro-6-((3-methylisoxazol-5-yl)methoxy)-1H-indol-2-yl)methyl)-3,3,3-trifluoropropanamide;
N-((5-chloro-6-((3-methylisoxazol-5-yl)methoxy)-1H-indol-2-yl)methyl)-1-(difluoromethyl)cyclopropane-1-carboxamide;
N-((5-fluoro-6-(thiazol-4-ylmethoxy)-1H-indol-2-yl)methyl)acetamide-2,2,2-d3;
2,2-difluoro-N-((5-fluoro-6-(thiazol-4-ylmethoxy)-1H-indol-2-yl)methyl)acetamide;
3,3,3-trifluoro-N-((5-fluoro-6-(thiazol-4-ylmethoxy)-1H-indol-2-yl)methyl)propanamide;
1-(difluoromethyl)-N-((5-fluoro-6-(thiazol-4-ylmethoxy)-1H-indol-2-yl)methyl)cyclopropane-1-carboxamide;
N-((5-chloro-6-(thiazol-4-ylmethoxy)-1H-indol-2-yl)methyl)acetamide-2,2,2-d3;
N-((5-chloro-6-((3-methylisoxazol-5-yl)methoxy)-1H-indol-2-yl)methyl)-1-fluorocyclopropane-1-carboxamide;
1-fluoro-N-((5-fluoro-6-((3-methylisoxazol-5-yl)methoxy)-1H-indol-2-yl)methyl)cyclopropane-1-carboxamide; (1R,2S)—N-((5-chloro-6-((3-methylisoxazol-5-yl)methoxy)-1H-indol-2-yl)methyl)-2-hydroxycyclopentane-1-carboxamide; (1S,2R)—N-((5-chloro-6-((3-methylisoxazol-5-yl)methoxy)-1H-indol-2-yl)methyl)-2-hydroxycyclopentane-1-carboxamide;
(R)—N-((5-chloro-6-((3-methylisoxazol-5-yl)methoxy)-1H-indol-2-yl)methyl)-1-methyl-2-oxopyrrolidine-3-carboxamide;
(S)—N-((5-chloro-6-((3-methylisoxazol-5-yl)methoxy)-1H-indol-2-yl)methyl)-1-methyl-2-oxopyrrolidine-3-carboxamide;
(R)—N-((5-chloro-6-((3-methylisoxazol-5-yl)methoxy)-1H-indol-2-yl)methyl)-1-methylazetidine-2-carboxamide;
(S)—N-((5-chloro-6-((3-methylisoxazol-5-yl)methoxy)-1H-indol-2-yl)methyl)-1-methylazetidine-2-carboxamide;
(S)—N-((5-chloro-6-((3-methylisoxazol-5-yl)methoxy)-1H-indol-2-yl)methyl)-1-methylpyrrolidine-2-carboxamide;
(R)—N-((5-chloro-6-((3-methylisoxazol-5-yl)methoxy)-1H-indol-2-yl)methyl)-1-methylpyrrolidine-2-carboxamide;
N-((5-chloro-6-((3-methylisoxazol-5-yl)methoxy)-1H-indol-2-yl)methyl)-2-(dimethylamino)acetamide;
N-((5-chloro-6-(thiazol-4-ylmethoxy)-1H-indol-2-yl)methyl)-2-(dimethylamino)acetamide;
(S)—N-((5-chloro-6-((3-methylisoxazol-5-yl)methoxy)-1H-indol-2-yl)methyl)-2-(difluoromethyl)azetidine-1-carboxamide;
(R)—N-((5-chloro-6-((3-methylisoxazol-5-yl)methoxy)-1H-indol-2-yl)methyl)-2-(difluoromethyl)azetidine-1-carboxamide;
N-((5-chloro-6-((3-methylisoxazol-5-yl)methoxy)-1H-indol-2-yl)methyl)-2-oxopropanamide;
N-((6-(2-(1H-1,2,3-triazol-1-yl)ethyl)-5-chloro-1H-indol-2-yl)methyl)-1-methylcyclopropane-1-carboxamide;
N-((6-(2-(2H-tetrazol-2-yl)ethyl)-5-chloro-1H-indol-2-yl)methyl)-1-methylcyclopropane-1-carboxamide;
N-((6-(2-(1H-tetrazol-1-yl)ethyl)-5-chloro-1H-indol-2-yl)methyl)-1-methylcyclopropane-1-carboxamide;
N-((6-(2-(2H-1,2,3-triazol-2-yl)ethyl)-5-chloro-1H-indol-2-yl)methyl)-1-methylcyclopropane-1-carboxamide;
N-((6-(2-(1H-pyrazol-1-yl)ethyl)-5-methoxy-1H-indol-2-yl)methyl)-1-methylcyclopropane-1-carboxamide;
N-((6-(2-(1H-1,2,3-triazol-1-yl)ethyl)-5-methoxy-1H-indol-2-yl)methyl)-1-methylcyclopropane-1-carboxamide;
N-((6-(2-(1H-imidazol-1-yl)ethyl)-5-chloro-1H-indol-2-yl)methyl)-1-methylcyclopropane-1-carboxamide;
N-((6-(2-(1H-pyrazol-1-yl)ethyl)-5-chloro-1H-indol-2-yl)methyl)-1-methylcyclopropane-1-carboxamide;
N-((6-(2-(2H-1,2,3-triazol-2-yl)ethyl)-5-methoxy-1H-indol-2-yl)methyl)-1-methylcyclopropane-1-carboxamide;
N-((5-fluoro-6-(2-(isoxazol-3-yl)ethyl)-1H-indol-2-yl)methyl)-1-methylcyclopropane-1-carboxamide;
N-((5-fluoro-6-(2-(5-methylisoxazol-3-yl)ethyl)-1H-indol-2-yl)methyl)-1-methylcyclopropane-1-carboxamide;
N-((5-fluoro-6-(2-(thiazol-4-yl)ethyl)-1H-indol-2-yl)methyl)-1-methylcyclopropane-1-carboxamide;
N-((5-chloro-6-(2-(isoxazol-3-yl)ethyl)-1H-indol-2-yl)methyl)azetidine-1-carboxamide;
N-((6-(2-(thiazol-4-yl)ethyl)-5-(trifluoromethoxy)-1H-indol-2-yl)methyl)azetidine-1-carboxamide;
N-((6-(2-(isoxazol-3-yl)ethyl)-5-(trifluoromethoxy)-1H-indol-2-yl)methyl)azetidine-1-carboxamide;
N-((6-(2-(isoxazol-3-yl)ethyl)-5-(trifluoromethoxy)-1H-indol-2-yl)methyl)pyrrolidine-1-carboxamide;
N-((5-chloro-6-(2-(thiazol-4-yl)ethyl)-1H-indol-2-yl)methyl)azetidine-1-carboxamide;
N-((5-fluoro-6-(2-(thiazol-4-yl)ethyl)-1H-indol-2-yl)methyl)azetidine-1-carboxamide;
N-((5-fluoro-6-(2-(thiazol-4-yl)ethyl)-1H-indol-2-yl)methyl)pyrrolidine-1-carboxamide;
N-((5-fluoro-6-(2-(isoxazol-3-yl)ethyl)-1H-indol-2-yl)methyl)azetidine-1-carboxamide;
N-((5-fluoro-6-(2-(isoxazol-3-yl)ethyl)-1H-indol-2-yl)methyl)pyrrolidine-1-carboxamide;
N-((5-(difluoromethoxy)-6-(thiazol-4-ylmethoxy)-1H-indol-2-yl)methyl)-1-methylcyclopropane-1-carboxamide;
N-((5-(difluoromethoxy)-6-(isoxazol-3-ylmethoxy)-1H-indol-2-yl)methyl)-1-methylcyclopropane-1-carboxamide;
N-((5-(difluoromethoxy)-6-((3-methylisoxazol-5-yl)methoxy)-1H-indol-2-yl)methyl)-1-methylcyclopropane-1-carboxamide;
N-((6-((1H-indol-2-yl)methoxy)-5-chloro-1H-indol-2-yl)methyl)-1-methylcyclopropane-1-carboxamide;
N-((5-chloro-6-((isoxazol-3-ylamino)methyl)-1H-indol-2-yl)methyl)-1-methylcyclopropane-1-carboxamide;
N-((5-chloro-6-((pyridin-2-ylamino)methyl)-1H-indol-2-yl)methyl)-1-methylcyclopropane-1-carboxamide;
N-((5-chloro-6-((pyridin-3-ylamino)methyl)-1H-indol-2-yl)methyl)-1-methylcyclopropane-1-carboxamide;
N-((5-fluoro-6-((isoxazol-3-ylamino)methyl)-1H-indol-2-yl)methyl)-1-methylcyclopropane-1-carboxamide;
N-((5-fluoro-6-((isoxazol-3-yloxy)methyl)-1H-indol-2-yl)methyl)-1-methylcyclopropane-1-carboxamide;
N-((5-chloro-6-((isoxazol-3-yloxy)methyl)-1H-indol-2-yl)methyl)-1-methylcyclopropane-1-carboxamide;
N-((5-chloro-6-(thiazol-4-ylmethoxy)-1H-indol-2-yl)methyl)-2-(methylamino)acetamide;
N-((5-chloro-6-((3-methylisoxazol-5-yl)methoxy)-1H-indol-2-yl)methyl)-2-(methylamino)acetamide;
(R)—N-((5-chloro-6-((3-methylisoxazol-5-yl)methoxy)-1H-indol-2-yl)methyl)azetidine-2-carboxamide;
2-amino-N-((5-chloro-6-((3-methylisoxazol-5-yl)methoxy)-1H-indol-2-yl)methyl)acetamide;
(R)—N-((5-chloro-6-((3-methylisoxazol-5-yl)methoxy)-1H-indol-2-yl)methyl)pyrrolidine-2-carboxamide;
1-((5-chloro-6-(thiazol-4-ylmethoxy)-1H-indol-2-yl)methyl)-3-(2-hydroxyethyl)urea;
ethyl ((5-chloro-6-(thiazol-4-ylmethoxy)-1H-indol-2-yl)methyl)carbamate;
methyl ((5-fluoro-6-((3-methylisoxazol-5-yl)methoxy)-1H-indol-2-yl)methyl)carbamate;
methyl ((5-fluoro-6-(thiazol-4-ylmethoxy)-1H-indol-2-yl)methyl)carbamate;
isopropyl ((5-chloro-6-(thiazol-4-ylmethoxy)-1H-indol-2-yl)methyl)carbamate;
methyl ((5-chloro-6-(thiazol-4-ylmethoxy)-1H-indol-2-yl)methyl)carbamate;
methyl ((5-chloro-6-((3-methylisoxazol-5-yl)methoxy)-1H-indol-2-yl)methyl)carbamate;
1-((1H-pyrazol-4-yl)methyl)-3-((5-chloro-6-(thiazol-4-ylmethoxy)-1H-indol-2-yl)methyl)-1-methylurea;
1-((1H-pyrazol-4-yl)methyl)-3-((6-(isoxazol-3-ylmethoxy)-1H-indol-2-yl)methyl)-1-methylurea;
1-((1H-pyrazol-4-yl)methyl)-1-methyl-3-((6-(thiazol-4-ylmethoxy)-1H-indol-2-yl)methyl)urea;
1-((1H-pyrazol-4-yl)methyl)-3-((5-chloro-6-(isoxazol-3-ylmethoxy)-1H-indol-2-yl)methyl)-1-methylurea;
N-((6-(2-(isoxazol-3-yl)ethyl)-5-methoxy-1H-indol-2-yl)methyl)-1-methylcyclopropane-1-carboxamide;
N-((5-methoxy-6-(2-(thiazol-4-yl)ethyl)-1H-indol-2-yl)methyl)-1-methylcyclopropane-1-carboxamide;
N-((5-chloro-6-(imidazo[1,2-a]pyridin-2-ylmethoxy)-1H-indol-2-yl)methyl)-1-methylcyclopropane-1-carboxamide;
N-((5-chloro-6-(cyclopropylmethoxy)-1H-indol-2-yl)methyl)-1-methylcyclopropane-1-carboxamide;
N-((5-chloro-6-(cyclobutylmethoxy)-1H-indol-2-yl)methyl)-1-methylcyclopropane-1-carboxamide;
N-((5-chloro-6-(2-cyclopropylethoxy)-1H-indol-2-yl)methyl)-1-methylcyclopropane-1-carboxamide;
1-((5-chloro-6-((3-methylisoxazol-5-yl)methoxy)-1H-indol-2-yl) methyl)-3-cyclopropylurea;
1-((5-chloro-6-((3-methylisoxazol-5-yl)methoxy)-1H-indol-2-yl)methyl)urea;
1-((5-chloro-6-((3-methylisoxazol-5-yl)methoxy)-1H-indol-2-yl)methyl)-3-isopropylurea;
(S)—N-((5-chloro-6-(thiazol-4-ylmethoxy)-1H-indol-2-yl)methyl)-2,3-difluoropropanamide;
(R)—N-((5-chloro-6-(thiazol-4-ylmethoxy)-1H-indol-2-yl)methyl)-2,3-difluoropropanamide;
1-methyl-N-((6-(thiazol-4-ylmethoxy)-3-(trifluoromethyl)-1H-indol-2-yl)methyl)cyclopropane-1-carboxamide;
N-((5-(2-cyanoethyl)-6-(thiazol-4-ylmethoxy)-1H-indol-2-yl)methyl)-1-methylcyclopropane-1-carboxamide;
N-((5-(2-cyanoethyl)-6-(isoxazol-3-ylmethoxy)-1H-indol-2-yl)methyl)-1-methylcyclopropane-1-carboxamide;
N-((5-(cyanomethyl)-6-(thiazol-4-ylmethoxy)-1H-indol-2-yl)methyl)-1-methylcyclopropane-1-carboxamide;
N-((5-(cyanomethyl)-6-(isoxazol-3-ylmethoxy)-1H-indol-2-yl)methyl)-1-methylcyclopropane-1-carboxamide;
N-((5-(difluoromethyl)-6-(thiazol-4-ylmethoxy)-1H-indol-2-yl)methyl)-1-methylcyclopropane-1-carboxamide;
N-((5-(difluoromethyl)-6-(isoxazol-3-ylmethoxy)-1H-indol-2-yl)methyl)-1-methylcyclopropane-1-carboxamide;
N-((5-(difluoromethyl)-6-((5-methylisoxazol-3-yl)methoxy)-1H-indol-2-yl)methyl)-1-methylcyclopropane-1-carboxamide;
1-methyl-N-((6-(thiazol-4-ylmethoxy)-5-(2,2,2-trifluoroethyl)-1H-indol-2-yl)methyl)cyclopropane-1-carboxamide;
N-((6-(isoxazol-3-ylmethoxy)-5-(2,2,2-trifluoroethyl)-1H-indol-2-yl)methyl)-1-methylcyclopropane-1-carboxamide;
N-((5-chloro-6-(2-(2,2-difluoroethoxy)ethoxy)-1H-indol-2-yl)methyl)acetamide;
N-((5-cyclopropyl-6-(thiazol-4-ylmethoxy)-1H-indol-2-yl)methyl)azetidine-1-carboxamide;
N-((5-cyclopropyl-6-(thiazol-4-ylmethoxy)-1H-indol-2-yl)methyl)pyrrolidine-1-carboxamide;
N-((5-cyclopropyl-6-(isoxazol-3-ylmethoxy)-1H-indol-2-yl)methyl)azetidine-1-carboxamide;
N-((5-cyclopropyl-6-(isoxazol-3-ylmethoxy)-1H-indol-2-yl)methyl)pyrrolidine-1-carboxamide;
N-((5-(1-fluorocyclopropyl)-6-(isoxazol-3-ylmethoxy)-1H-indol-2-yl)methyl)-1-methylcyclopropane-1-carboxamide;
N-((5-(1-fluorocyclopropyl)-6-(thiazol-4-ylmethoxy)-1H-indol-2-yl)methyl)-1-methylcyclopropane-1-carboxamide;
N-((5-(2,2-difluorocyclopropyl)-6-(thiazol-4-ylmethoxy)-1H-indol-2-yl)methyl)-1-methylcyclopropane-1-carboxamide;
(S)—N-((5-(2,2-difluorocyclopropyl)-6-(isoxazol-3-ylmethoxy)-1H-indol-2-yl)methyl)-1-methylcyclopropane-1-carboxamide;
(R)—N-((5-(2,2-difluorocyclopropyl)-6-(isoxazol-3-ylmethoxy)-1H-indol-2-yl)methyl)-1-methylcyclopropane-1-carboxamide;
(5-chloro-6-(2-(isoxazol-3-yl)ethyl)-1H-indol-2-yl)methanamine;
N-((5-(isoxazol-5-yl)-6-(thiazol-4-ylmethoxy)-1H-indol-2-yl)methyl)-1-methylcyclopropane-1-carboxamide;
1-methyl-N-((5-(oxazol-5-yl)-6-(thiazol-4-ylmethoxy)-1H-indol-2-yl)methyl)cyclopropane-1-carboxamide;
N-((5-chloro-6-((3-methylisoxazol-5-yl)methoxy)-1H-indol-2-yl)methyl)-2,2,2-trifluoroacetamide;
N-((5-chloro-6-((5-(methyl-d3)isoxazol-3-yl)methoxy)-1H-indol-2-yl)methyl)-1-methylcyclopropane-1-carboxamide;
N-((5-fluoro-6-((5-(methyl-d3)isoxazol-3-yl)methoxy)-1H-indol-2-yl)methyl)-1-methylcyclopropane-1-carboxamide;
(2S,4R)—N-((5-chloro-6-((3-methylisoxazol-5-yl)methoxy)-1H-indol-2-yl)methyl)-4-fluoropyrrolidine-2-carboxamide;
(S)—N-((5-chloro-6-((3-methylisoxazol-5-yl)methoxy)-1H-indol-2-yl)methyl)-4,4-difluoropyrrolidine-2-carboxamide;
(2S,4R)—N-((5-chloro-6-((3-methylisoxazol-5-yl)methoxy)-1H-indol-2-yl)methyl)-4-fluoro-1-methylpyrrolidine-2-carboxamide;
(S)—N-((5-chloro-6-((3-methylisoxazol-5-yl)methoxy)-1H-indol-2-yl)methyl)-4,4-difluoro-1-methylpyrrolidine-2-carboxamide;
(S)—N-((5-chloro-6-(thiazol-4-ylmethoxy)-1H-indol-2-yl)methyl)-1-methylazetidine-2-carboxamide;
(R)—N-((5-chloro-6-(thiazol-4-ylmethoxy)-1H-indol-2-yl)methyl)-1-methylazetidine-2-carboxamide; or a stereoisomer or tautomer thereof, or a pharmaceutically acceptable salt of any of the foregoing.

In some embodiments, provided herein is a compound of formula (I′), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
N-((6-(benzylamino)-1H-indol-2-yl)methyl)propionamide;
N-((6-(benzyl(methyl)amino)-1H-indol-2-yl)methyl)-1-methylcyclopropane-1-carboxamide;
N-((6-(benzylamino)-1H-indol-2-yl)methyl)-1-methylcyclopropanecarboxamide;
1-methyl-N-((6-((pyridin-2-ylmethyl)amino)-1H-indol-2-yl)methyl)cyclopropane-1-carboxamide;
N-((6-(benzyloxy)-1H-indol-2-yl)methyl)-1-methylcyclopropanecarboxamide;
N-((5-fluoro-6-(isoxazol-3-ylmethoxy)-1H-indol-2-yl) methyl)-1-methylcyclopropane-1-carboxamide;
N-((5-fluoro-6-(oxazol-4-ylmethoxy)-1H-indol-2-yl)methyl)-1-methylcyclopropane-1-carboxamide;
N-((5-fluoro-6-(thiazol-4-ylmethoxy)-1H-indol-2-yl)methyl)-1-methylcyclopropane-1-carboxamide;
N-((5-fluoro-6-(thiazol-5-ylmethoxy)-1H-indol-2-yl)methyl)-1-methylcyclopropane-1-carboxamide;
N-((6-(isoxazol-3-ylmethoxy)-5-methyl-1H-indol-2-yl)methyl)-1-methylcyclopropane-1-carboxamide;
1-methyl-N-((5-methyl-6-(thiazol-4-ylmethoxy)-1H-indol-2-yl)methyl)cyclopropane-1-carboxamide;
N-((5-fluoro-6-((3-methylisoxazol-5-yl)methoxy)-1H-indol-2-yl)methyl)-1-methylcyclopropane-1-carboxamide;
N-((5-fluoro-6-((2-methylthiazol-5-yl)methoxy)-1H-indol-2-yl)methyl)-1-methylcyclopropane-1-carboxamide;
1-methyl-N-((5-methyl-6-((5-methylisoxazol-3-yl)methoxy)-1H-indol-2-yl)methyl)cyclopropane-1-carboxamide;
N-((5-chloro-4-fluoro-6-(isoxazol-3-ylmethoxy)-1H-indol-2-yl)methyl)-1-methylcyclopropane-1-carboxamide;
N-((5-chloro-7-fluoro-6-(isoxazol-3-ylmethoxy)-1H-indol-2-yl)methyl)-1-methylcyclopropane-1-carboxamide;
N-((5-chloro-4-fluoro-6-(thiazol-4-ylmethoxy)-1H-indol-2-yl)methyl)-1-methylcyclopropane-1-carboxamide;
N-((5-chloro-7-fluoro-6-(thiazol-4-ylmethoxy)-1H-indol-2-yl)methyl)-1-methylcyclopropane-1-carboxamide;
N-((7-fluoro-6-(thiazol-4-ylmethoxy)-1H-indol-2-yl)methyl)-1-methylcyclopropane-1-carboxamide;
N-((6-(isoxazol-3-ylmethoxy)-1H-indol-2-yl)methyl)-1-methylcyclopropane-1-carboxamide;
N-((5-chloro-6-((5-methylpyridin-2-yl)methoxy)-1H-indol-2-yl)methyl)-1-methylcyclopropane-1-carboxamide;
N-((5-chloro-6-((4-methylpyridin-2-yl)methoxy)-1H-indol-2-yl)methyl)-1-methylcyclopropane-1-carboxamide;
N-((5-chloro-6-((5-methylpyridin-3-yl)methoxy)-1H-indol-2-yl)methyl)-1-methylcyclopropane-1-carboxamide;
N-((5-chloro-6-((4-methylthiazol-2-yl)methoxy)-1H-indol-2-yl)methyl)-1-methylcyclopropane-1-carboxamide;
N-((5-chloro-6-(thiophen-3-ylmethoxy)-1H-indol-2-yl)methyl)-1-methylcyclopropane-1-carboxamide;
N-((5-chloro-6-((3-fluoropyridin-2-yl)methoxy)-1H-indol-2-yl)methyl)-1-methylcyclopropane-1-carboxamide;
N-((4-fluoro-6-(isoxazol-3-ylmethoxy)-1H-indol-2-yl)methyl)-1-methylcyclopropane-1-carboxamide;
N-((5-chloro-6-((5-fluoropyridin-2-yl)methoxy)-1H-indol-2-yl)methyl)-1-methylcyclopropane-1-carboxamide;
N-((4-fluoro-6-(thiazol-4-ylmethoxy)-1H-indol-2-yl)methyl)-1-methylcyclopropane-1-carboxamide;
N-((5-chloro-6-((2-fluoropyridin-3-yl)methoxy)-1H-indol-2-yl)methyl)-1-methylcyclopropane-1-carboxamide;
N-((5-chloro-6-((6-fluoropyridin-3-yl)methoxy)-1H-indol-2-yl)methyl)-1-methylcyclopropane-1-carboxamide;
N-((5-chloro-6-((6-fluoropyridin-2-yl)methoxy)-1H-indol-2-yl)methyl)-1-methylcyclopropane-1-carboxamide;
N-((6-((6-fluoropyridin-2-yl)methoxy)-1H-indol-2-yl)methyl)-1-methylcyclopropane-1-carboxamide;
N-((6-((6-fluoropyridin-3-yl)methoxy)-1H-indol-2-yl)methyl)-1-methylcyclopropane-1-carboxamide;
N-((5-chloro-6-((4-fluoropyridin-2-yl)methoxy)-1H-indol-2-yl)methyl)-1-methylcyclopropane-1-carboxamide;
N-((5-chloro-6-((5-fluoropyridin-3-yl)methoxy)-1H-indol-2-yl)methyl)-1-methylcyclopropane-1-carboxamide;
N-((5-chloro-6-((6-methylpyridin-3-yl)methoxy)-1H-indol-2-yl)methyl)-1-methylcyclopropane-1-carboxamide;
N-((5-chloro-6-(isothiazol-3-ylmethoxy)-1H-indol-2-yl)methyl)-1-methylcyclopropane-1-carboxamide;
N-((5-chloro-6-(pyridin-3-ylmethoxy)-1H-indol-2-yl)methyl)-1-methylcyclopropane-1-carboxamide;
N-((5-chloro-6-(thiazol-2-ylmethoxy)-1H-indol-2-yl)methyl)-1-methylcyclopropane-1-carboxamide;
N-((5-chloro-6-(pyridin-2-ylmethoxy)-1H-indol-2-yl)methyl)-1-methylcyclopropane-1-carboxamide;
N-((5-chloro-6-((6-methylpyridin-2-yl)methoxy)-1H-indol-2-yl)methyl)-1-methylcyclopropane-1-carboxamide;
1-methyl-N-((6-(thiazol-2-ylmethoxy)-1H-indol-2-yl)methyl)cyclopropane-1-carboxamide;
N-((6-((4-chlorobenzyl)oxy)-1H-indol-2-yl)methyl)-1-methylcyclopropane-1-carboxamide;
N-((5-chloro-6-((5-methylisoxazol-3-yl)methoxy)-1H-indol-2-yl)methyl)-1-methylcyclopropane-1-carboxamide;
N-((5-chloro-6-(isoxazol-3-ylmethoxy)-1H-indol-2-yl)methyl)-1-methylcyclopropane-1-carboxamide;
N-((6-((5-methylisoxazol-3-yl)methoxy)-1H-indol-2-yl)methyl)propionamide;
1-methyl-N-((6-(thiazol-5-ylmethoxy)-1H-indol-2-yl)methyl)cyclopropane-1-carboxamide;
1-methyl-N-((6-(oxazol-4-ylmethoxy)-1H-indol-2-yl)methyl)cyclopropane-1-carboxamide;
1-methyl-N-((6-((5-methylisoxazol-3-yl)methoxy)-1H-indol-2-yl)methyl)cyclopropane-1-carboxamide;
1-methyl-N-((6-(pyrimidin-2-ylmethoxy)-1H-indol-2-yl)methyl)cyclopropane-1-carboxamide;
1-methyl-N-((6-(pyrimidin-5-ylmethoxy)-1H-indol-2-yl)methyl)cyclopropane-1-carboxamide;
1-methyl-N-((6-(pyrimidin-4-ylmethoxy)-1H-indol-2-yl)methyl)cyclopropane-1-carboxamide;
1-methyl-N-((6-(pyridin-2-ylmethoxy)-1H-indol-2-yl)methyl)cyclopropane-1-carboxamide;
1-methyl-N-((6-(pyridin-3-ylmethoxy)-1H-indol-2-yl)methyl)cyclopropane-1-carboxamide;
1-methyl-N-((6-(pyridin-4-ylmethoxy)-1H-indol-2-yl)methyl)cyclopropane-1-carboxamide;
N-((5-fluoro-6-(oxazol-5-ylmethoxy)-1H-indol-2-yl) methyl)-1-methylcyclopropane-1-carboxamide;
1-methyl-N-((6-((5-methylisoxazol-3-yl)methoxy)-5-(trifluoromethyl)-1H-indol-2-yl)methyl)cyclopropanecarboxamide;
N-((6-(isoxazol-3-ylmethoxy)-5-(trifluoromethyl)-1H-indol-2-yl)methyl)-1-methylcyclopropane-1-carboxamide;
1-methyl-N-((6-(thiazol-4-ylmethoxy)-5-(trifluoromethyl)-1H-indol-2-yl)methyl)cyclopropane-1-carboxamide;
N-((5-ethyl-6-(isoxazol-3-ylmethoxy)-1H-indol-2-yl)methyl)-1-methylcyclopropane-1-carboxamide;
N-((6-(isoxazol-5-ylmethoxy)-1H-indol-2-yl)methyl)-1-methylcyclopropanecarboxamide;
N-((5-chloro-6-((5-methylthiazol-2-yl)methoxy)-1H-indol-2-yl)methyl)-1-methylcyclopropane-1-carboxamide;
N-((5-chloro-6-(furan-3-ylmethoxy)-1H-indol-2-yl)methyl)-1-methylcyclopropane-1-carboxamide;
N-((5-chloro-6-(thiophen-2-ylmethoxy)-1H-indol-2-yl)methyl)-1-methylcyclopropane-1-carboxamide;
N-((5-chloro-6-(furan-2-ylmethoxy)-1H-indol-2-yl)methyl)-1-methylcyclopropane-1-carboxamide;
N-((5-chloro-6-(2-(thiazol-4-yl)ethoxy)-1H-indol-2-yl)methyl)-1-methylcyclopropane-1-carboxamide;
N-((6-(isothiazol-4-ylmethoxy)-1H-indol-2-yl)methyl)-1-methylcyclopropane-1-carboxamide;
N-((5-chloro-6-(isothiazol-4-ylmethoxy)-1H-indol-2-yl)methyl)-1-methylcyclopropane-1-carboxamide;
N-((5-chloro-6-((5-methyloxazol-2-yl)methoxy)-1H-indol-2-yl)methyl)-1-methylcyclopropane-1-carboxamide;
N-((5-chloro-6-((4-methyloxazol-2-yl)methoxy)-1H-indol-2-yl)methyl)-1-methylcyclopropane-1-carboxamide;
N-((5-fluoro-6-((5-(trifluoromethyl)isoxazol-3-yl)methoxy)-1H-indol-2-yl)methyl)-1-methylcyclopropane-1-carboxamide;
N-((5-fluoro-6-((5-methyloxazol-2-yl)methoxy)-1H-indol-2-yl)methyl)-1-methylcyclopropane-1-carboxamide;
N-((5-fluoro-6-((5-methyloxazol-2-yl)methoxy)-1H-indol-2-yl)methyl)-1-methylcyclopropane-1-carboxamide;
N-((6-(2-(isoxazol-3-yl)ethyl)-1H-indol-2-yl)methyl)-1-methylcyclopropanecarboxamide;
1-methyl-N-((6-(2-(thiazol-4-yl)ethyl)-1H-indol-2-yl)methyl)cyclopropane-1-carboxamide;
1-methyl-N-((6-(2-(5-methylisoxazol-3-yl)ethyl)-1H-indol-2-yl)methyl)cyclopropane-1-carboxamide;
1-methyl-N-((6-((5-methylisoxazol-3-yl)oxy)-1H-indol-2-yl)methyl)cyclopropanecarboxamide;
N-((5-chloro-6-(isoxazol-3-ylmethoxy)-1H-indol-2-yl)methyl)azetidine-1-carboxamide;
N-((5-chloro-6-(thiazol-4-ylmethoxy)-1H-indol-2-yl)methyl)-3-methylazetidine-1-carboxamide;
N-((5-chloro-6-(thiazol-4-ylmethoxy)-1H-indol-2-yl)methyl)azetidine-1-carboxamide;
N-((5-chloro-6-(thiazol-4-ylmethoxy)-1H-indol-2-yl)methyl)pyrrolidine-1-carboxamide;
N-((5-chloro-6-((3-methylisoxazol-5-yl)methoxy)-1H-indol-2-yl)methyl)pyrrolidine-1-carboxamide;
N-((5-chloro-6-(isoxazol-3-ylmethoxy)-1H-indol-2-yl)methyl)morpholine-4-carboxamide;
N-((5-chloro-6-(isoxazol-3-ylmethoxy)-1H-indol-2-yl)methyl)piperidine-1-carboxamide;
N-((5-chloro-6-(isoxazol-3-ylmethoxy)-1H-indol-2-yl)methyl)-2-methylpyrrolidine-1-carboxamide;
N-((5-chloro-6-(isoxazol-3-ylmethoxy)-1H-indol-2-yl)methyl)-3-methylpyrrolidine-1-carboxamide;
N-((5-fluoro-6-(isoxazol-3-ylmethoxy)-1H-indol-2-yl)methyl)piperidine-1-carboxamide;
N-((5-fluoro-6-(isoxazol-3-ylmethoxy)-1H-indol-2-yl)methyl)azetidine-1-carboxamide;
N-((5-fluoro-6-(isoxazol-3-ylmethoxy)-1H-indol-2-yl)methyl)-2-methylpyrrolidine-1-carboxamide;
N-((5-fluoro-6-(isoxazol-3-ylmethoxy)-1H-indol-2-yl)methyl)-3-methylpyrrolidine-1-carboxamide;
N-((5-fluoro-6-(thiazol-4-ylmethoxy)-1H-indol-2-yl)methyl)azetidine-1-carboxamide;
N-((5-chloro-6-(thiazol-4-ylmethoxy)-1H-indol-2-yl)methyl)-3-fluoropyrrolidine-1-carboxamide;
N-((6-(isothiazol-3-ylmethoxy)-1H-indol-2-yl)methyl)-1-methylcyclopropanecarboxamide;
(N-((5-chloro-6-((5-ethylisoxazol-3-yl)methoxy)-1H-indol-2-yl)methyl)-1-methylcyclopropanecarboxamide;
N-((5-chloro-6-((5-(difluoromethyl)isoxazol-3-yl)methoxy)-1H-indol-2-yl)methyl)-1-methylcyclopropane-1-carboxamide;
N-((6-((5-(difluoromethyl)isoxazol-3-yl)methoxy)-1H-indol-2-yl)methyl)-1-methylcyclopropane-1-carboxamide;
N-((6-((2-fluoropyridin-3-yl)methoxy)-1H-indol-2-yl)methyl)-1-methylcyclopropane-1-carboxamide formate;
N-((5-chloro-6-(2-(5-methylisoxazol-3-yl)ethoxy)-1H-indol-2-yl)methyl)-1-methylcyclopropane-1-carboxamide;
N-((5-chloro-6-((5-cyclopropylisoxazol-3-yl)methoxy)-1H-indol-2-yl)methyl)-1-methylcyclopropane-1-carboxamide;
N-((5-chloro-6-((6-(trifluoromethyl)pyridin-2-yl)methoxy)-1H-indol-2-yl)methyl)-1-methylcyclopropane-1-carboxamide;
N-((5-chloro-6-((5-isopropylisoxazol-3-yl)methoxy)-1H-indol-2-yl)methyl)-1-methylcyclopropane-1-carboxamide;
N-((5-chloro-6-(2-(isoxazol-3-yl)ethoxy)-1H-indol-2-yl)methyl)-1-methylcyclopropane-1-carboxamide;
N-((6-(isoxazol-3-ylmethoxy)-1H-indol-2-yl)methyl)pyrrolidine-1-carboxamide;
N-((6-(oxazol-4-ylmethoxy)-1H-indol-2-yl)methyl)pyrrolidine-1-carboxamide;
N-((6-(thiazol-5-ylmethoxy)-1H-indol-2-yl)methyl)pyrrolidine-1-carboxamide;
1-((6-(isoxazol-3-ylmethoxy)-1H-indol-2-yl)methyl)-3-methylurea;
1-methyl-3-((6-(oxazol-4-ylmethoxy)-1H-indol-2-yl)methyl)urea;
1-((5-chloro-6-(thiazol-5-ylmethoxy)-1H-indol-2-yl)methyl)-3-methylurea;
1-((5-chloro-6-(isoxazol-3-ylmethoxy)-1H-indol-2-yl)methyl)-3-methylurea;
1-((5-chloro-6-(oxazol-4-ylmethoxy)-1H-indol-2-yl)methyl)-3-methylurea;
N-((5-chloro-6-(isoxazol-3-ylmethoxy)-1H-indol-2-yl)methyl)pyrrolidine-1-carboxamide;
N-((5-chloro-6-(oxazol-4-ylmethoxy)-1H-indol-2-yl)methyl)pyrrolidine-1-carboxamide;
N-((5-fluoro-6-(thiazol-4-ylmethoxy)-1H-indol-2-yl)methyl)pyrrolidine-1-carboxamide;
N-((5-fluoro-6-(oxazol-4-ylmethoxy)-1H-indol-2-yl)methyl)pyrrolidine-1-carboxamide;
N-((5-fluoro-6-(isoxazol-3-ylmethoxy)-1H-indol-2-yl)methyl)pyrrolidine-1-carboxamide;
N-((5-fluoro-6-((5-methylisoxazol-3-yl)methoxy)-1H-indol-2-yl)methyl)pyrrolidine-1-carboxamide;
N-((5-fluoro-6-((3-methylisoxazol-5-yl)methoxy)-1H-indol-2-yl)methyl)pyrrolidine-1-carboxamide;
N-((5-chloro-6-(thiazol-5-ylmethoxy)-1H-indol-2-yl) methyl) pyrrolidine-1-carboxamide;
N-((5-chloro-6-((5-methyloxazol-2-yl)methoxy)-1H-indol-2-yl)methyl)pyrrolidine-1-carboxamide;
N-((6-(isoxazol-3-ylmethoxy)-1H-indol-2-yl)methyl)-3-methylpyrrolidine-1-carboxamide;
N-((6-(isoxazol-3-ylmethoxy)-1H-indol-2-yl)methyl)azetidine-1-carboxamide;
N-((6-(isoxazol-3-ylmethoxy)-1H-indol-2-yl)methyl)piperidine-1-carboxamide;
N-((6-(isoxazol-3-ylmethoxy)-1H-indol-2-yl)methyl)-2-methylpyrrolidine-1-carboxamide;
N-((5-chloro-6-((5-methylisoxazol-3-yl)methoxy)-1H-indol-2-yl)methyl)-1-(trifluoromethyl)cyclopropane-1-carboxamide;
N-((5-fluoro-6-(thiazol-4-ylmethoxy)-1H-indol-2-yl)methyl)propionamide;
N-((5-fluoro-6-(thiazol-4-ylmethoxy)-1H-indol-2-yl)methyl)-1-(trifluoromethyl)cyclopropane-1-carboxamide;
N-((5-chloro-6-((5-methylisoxazol-3-yl)methoxy)-1H-indol-2-yl)methyl)propionamide;
N-((5-chloro-6-(isoxazol-3-ylmethoxy)-1H-indol-2-yl)methyl)propionamide;
N-((5-chloro-6-((5-methylisoxazol-3-yl)methoxy)-1H-indol-2-yl)methyl)isobutyramide;
N-((5-chloro-6-(isoxazol-3-ylmethoxy)-1H-indol-2-yl)methyl)cyclopropanecarboxamide;
N-((5-chloro-6-(isoxazol-3-ylmethoxy)-1H-indol-2-yl)methyl)isobutyramide;
N-((5-chloro-6-((5-methylisoxazol-3-yl)methoxy)-1H-indol-2-yl)methyl)cyclopropanecarboxamide;
N-((5-chloro-6-(isoxazol-3-ylmethoxy)-1H-indol-2-yl)methyl)-1-(trifluoromethyl)cyclopropane-1-carboxamide;
N-((5-fluoro-6-(thiazol-4-ylmethoxy)-1H-indol-2-yl)methyl)isobutyramide;
N-((5-fluoro-6-(thiazol-4-ylmethoxy)-1H-indol-2-yl)methyl)cyclopropanecarboxamide;
N-((5-fluoro-6-(isoxazol-3-ylmethoxy)-1H-indol-2-yl)methyl)propionamide;
N-((5-fluoro-6-(isoxazol-3-ylmethoxy)-1H-indol-2-yl)methyl)isobutyramide;
N-((5-fluoro-6-(isoxazol-3-ylmethoxy)-1H-indol-2-yl)methyl)-1-(trifluoromethyl)cyclopropane-1-carboxamide;
N-((5-fluoro-6-(isoxazol-3-ylmethoxy)-1H-indol-2-yl)methyl)cyclopropanecarboxamide;
N-((5-chloro-6-(thiazol-4-ylmethoxy)-1H-indol-2-yl)methyl)acetamide;
N-((5-chloro-6-((5-methylisoxazol-3-yl)methoxy)-1H-indol-2-yl)methyl)acetamide;
N-((5-chloro-6-(isoxazol-3-ylmethoxy)-1H-indol-2-yl)methyl)acetamide;
N-((5-chloro-6-(thiazol-4-ylmethoxy)-1H-indol-2-yl)methyl)-1-(trifluoromethyl)cyclopropane-1-carboxamide;
N-((5-chloro-6-(thiazol-4-ylmethoxy)-1H-indol-2-yl)methyl)isobutyramide;
N-((5-chloro-6-(thiazol-4-ylmethoxy)-1H-indol-2-yl)methyl)propionamide;
N-((5-chloro-6-(thiazol-4-ylmethoxy)-1H-indol-2-yl)methyl)cyclopropanecarboxamide;
N-((5-chloro-6-(hydroxymethyl)-1H-indol-2-yl)methyl)-1-methylcyclopropanecarboxamide;
N-((5-chloro-6-(2-(thiazol-4-yl)ethyl)-1H-indol-2-yl)methyl)-1-methylcyclopropane-1-carboxamide;
N-((5-chloro-6-(2-(isoxazol-3-yl)ethyl)-1H-indol-2-yl)methyl)-1-methylcyclopropane-1-carboxamide;
N-((5-chloro-6-((isoxazol-3-ylmethyl)amino)-1H-indol-2-yl)methyl)-1-methylcyclopropane-1-carboxamide;
N-((6-((isoxazol-3-ylmethyl)amino)-1H-indol-2-yl)methyl)-1-methylcyclopropane-1-carboxamide;
1-methyl-N-((6-(1-(5-methylisoxazol-3-yl) propan-2-yl)-1H-indol-2-yl) methyl) cyclopropane-1-carboxamide;
trans-1-methyl-N-((6-(2-phenylcyclopropyl)-1H-indol-2-yl)methyl)cyclopropane-1-carboxamide;
N-((6-(isoxazol-3-ylmethoxy)-3-methyl-1H-indol-2-yl)methyl)-1-methylcyclopropane-1-carboxamide;
N-((6-(benzyloxy)-3-methyl-1H-indol-2-yl)methyl)-1-methylcyclopropane-1-carboxamide;
N-((3-chloro-6-(isoxazol-3-ylmethoxy)-1H-indol-2-yl)methyl)-1-methylcyclopropane-1-carboxamide;
N-((6-(benzyloxy)-3-chloro-1H-indol-2-yl)methyl)-1-methylcyclopropane-1-carboxamide;
N-((5-chloro-6-(isoxazol-3-ylmethyl)-1H-indol-2-yl)methyl)-1-methylcyclopropanecarboxamide;
1-methyl-N-((6-((5-methylisoxazol-3-yl)methyl)-1H-indol-2-yl)methyl)cyclopropanecarboxamide;
N-((5-chloro-6-(oxazol-4-ylmethoxy)-1H-indol-2-yl)methyl)-1-methylcyclopropane-1-carboxamide;
1-methyl-N-((6-(2-(methylamino)-2-oxoethoxy)-1H-indol-2-yl)methyl)cyclopropane-1-carboxamide;
1-methyl-N-((6-(oxazol-5-ylmethoxy)-1H-indol-2-yl)methyl)cyclopropane-1-carboxamide;
1-methyl-N-((6-(thiazol-4-ylmethoxy)-1H-indol-2-yl)methyl)cyclopropane-1-carboxamide;
1-methyl-N-((6-((5-methyl-1,3,4-thiadiazol-2-yl)methoxy)-1H-indol-2-yl)methyl)cyclopropane-1-carboxamide;
N-((6-((1,2,5-thiadiazol-3-yl)methoxy)-1H-indol-2-yl)methyl)-1-methylcyclopropane-1-carboxamide;
1-methyl-N-((6-((5-methyl-1,3,4-oxadiazol-2-yl)methoxy)-1H-indol-2-yl)methyl)cyclopropane-1-carboxamide;
1-methyl-N-((6-((1-methyl-1H-1,2,3-triazol-4-yl)methoxy)-1H-indol-2-yl)methyl)cyclopropane-1-carboxamide;
1-methyl-N-((6-((3-methyl-1,2,4-oxadiazol-5-yl)methoxy)-1H-indol-2-yl)methyl)cyclopropane-1-carboxamide;
N-((6-((1,2,3-thiadiazol-4-yl)methoxy)-1H-indol-2-yl)methyl)-1-methylcyclopropane-1-carboxamide;
N-((6-((1,3,4-thiadiazol-2-yl)methoxy)-1H-indol-2-yl)methyl)-1-methylcyclopropane-1-carboxamide;
N-((5-chloro-6-((2-methyl-2H-1,2,3-triazol-4-yl)methoxy)-1H-indol-2-yl)methyl)-1-methylcyclopropane-1-carboxamide;
1-methyl-N-((6-((2-methyl-2H-1,2,3-triazol-4-yl)methoxy)-1H-indol-2-yl)methyl)cyclopropane-1-carboxamide;
1-methyl-N-((6-((2-methyl-2H-tetrazol-5-yl)methoxy)-1H-indol-2-yl)methyl)cyclopropane-1-carboxamide;
N-((5-chloro-6-((3-methylisoxazol-5-yl)methoxy)-1H-indol-2-yl)methyl)-1-methylcyclopropane-1-carboxamide;
N-((5-chloro-6-((2-methylthiazol-5-yl)methoxy)-1H-indol-2-yl)methyl)-1-methylcyclopropane-1-carboxamide;
N-((5-chloro-6-(thiazol-5-ylmethoxy)-1H-indol-2-yl)methyl)-1-methylcyclopropane-1-carboxamide;
1-methyl-N-((6-((2-methylthiazol-5-yl)methoxy)-1H-indol-2-yl)methyl)cyclopropane-1-carboxamide;
1-methyl-N-((6-((2-methylthiazol-4-yl)methoxy)-1H-indol-2-yl)methyl)cyclopropane-1-carboxamide;
1-methyl-N-((6-((2-methyloxazol-4-yl)methoxy)-1H-indol-2-yl)methyl)cyclopropane-1-carboxamide;
1-methyl-N-((6-((3-methylisoxazol-5-yl)methoxy)-1H-indol-2-yl)methyl)cyclopropane-1-carboxamide;
N-((5-chloro-6-((2-methyloxazol-4-yl)methoxy)-1H-indol-2-yl)methyl)-1-methylcyclopropane-1-carboxamide;
N-((6-((1,2,5-thiadiazol-3-yl)methoxy)-5-chloro-1H-indol-2-yl)methyl)-1-methylcyclopropane-1-carboxamide;
N-((5-chloro-6-(thiazol-4-ylmethoxy)-1H-indol-2-yl)methyl)-1-methylcyclopropane-1-carboxamide;
N-((6-((5-ethylisoxazol-3-yl)methoxy)-1H-indol-2-yl)methyl)-1-methylcyclopropane-1-carboxamide;
N-((5-isopropyl-6-(thiazol-4-ylmethoxy)-1H-indol-2-yl)methyl)-1-methylcyclopropane-1-carboxamide;
N-((6-(cyclopentylmethoxy)-1H-indol-2-yl)methyl)-1-methylcyclopropane-1-carboxamide;
1-methyl-N-((6-((tetrahydrofuran-3-yl)methoxy)-1H-indol-2-yl)methyl)cyclopropane-1-carboxamide;
N-((6-(bicyclo[1.1.1]pentan-1-ylmethoxy)-1H-indol-2-yl)methyl)-1-methylcyclopropane-1-carboxamide;
N-((6-(2-(isoxazol-3-yl)ethoxy)-1H-indol-2-yl)methyl)-1-methylcyclopropane-1-carboxamide;
1-methyl-N-((6-(2-(thiazol-4-yl)ethoxy)-1H-indol-2-yl)methyl)cyclopropane-1-carboxamide;
N-((6-(benzofuran-6-ylmethoxy)-1H-indol-2-yl)methyl)-1-methylcyclopropane-1-carboxamide;
N-((6-(2-(bicyclo[1.1.1]pentan-1-yl)ethoxy)-1H-indol-2-yl)methyl)-1-methylcyclopropane-1-carboxamide;
N-((6-((1H-1,2,3-triazol-4-yl)methoxy)-1H-indol-2-yl)methyl)-1-methylcyclopropane-1-carboxamide;
1-methyl-N-((6-(2-(5-methylisoxazol-3-yl)propyl)-1H-indol-2-yl)methyl)cyclopropanecarboxamide;
N-((6-((isoxazol-3-ylmethyl)thio)-1H-indol-2-yl)methyl)-1-methylcyclopropanecarboxamide;
N-((5-chloro-6-((isoxazol-3-ylmethyl)thio)-1H-indol-2-yl)methyl)-1-methylcyclopropanecarboxamide;
N-((5-chloro-6-((3-hydroxybenzyl) oxy)-1H-indol-2-yl) methyl)-1-methylcyclopropane-1-carboxamide;
N-((6-(isoxazol-3-ylmethoxy)-1H-indol-2-yl)methyl)morpholine-4-carboxamide;
3-fluoro-N-((6-(isoxazol-3-ylmethoxy)-1H-indol-2-yl)methyl)pyrrolidine-1-carboxamide;
N-((7-fluoro-6-(isoxazol-3-ylmethoxy)-1H-indol-2-yl)methyl)-1-methylcyclopropane-1-carboxamide;
N-((5-ethyl-6-(thiazol-4-ylmethoxy)-1H-indol-2-yl)methyl)-1-methylcyclopropane-1-carboxamide;
N-((5-chloro-6-((2-hydroxybenzyl)oxy)-1H-indol-2-yl)methyl)-1-methylcyclopropane-1-carboxamide;
N-((5-isopropyl-6-(isoxazol-3-ylmethoxy)-1H-indol-2-yl)methyl)-1-methylcyclopropane-1-carboxamide;
N-((5-fluoro-6-((5-methylisoxazol-3-yl)methoxy)-1H-indol-2-yl)methyl)acetamide;
N-((5-chloro-6-(oxazol-4-ylmethoxy)-1H-indol-2-yl)methyl)acetamide;
N-((5-fluoro-6-(isoxazol-3-ylmethoxy)-1H-indol-2-yl)methyl)acetamide;
N-((5-fluoro-6-(thiazol-4-ylmethoxy)-1H-indol-2-yl)methyl)acetamide;
1-((5-chloro-6-(thiazol-4-ylmethoxy)-1H-indol-2-yl)methyl)-3-methylurea;
1-((5-fluoro-6-((3-methylisoxazol-5-yl)methoxy)-1H-indol-2-yl)methyl)-3-methylurea;
1-((5-chloro-6-((3-methylisoxazol-5-yl)methoxy)-1H-indol-2-yl)methyl)-3-methylurea;
1-((5-fluoro-6-((5-methylisoxazol-3-yl)methoxy)-1H-indol-2-yl)methyl)-3-methylurea;
1-((5-chloro-6-((5-methylisoxazol-3-yl)methoxy)-1H-indol-2-yl)methyl)-3-methylurea;
1-((5-fluoro-6-(thiazol-4-ylmethoxy)-1H-indol-2-yl)methyl)-3-methylurea;
1-((5-fluoro-6-(oxazol-4-ylmethoxy)-1H-indol-2-yl)methyl)-3-methylurea;
1-((5-fluoro-6-(isoxazol-3-ylmethoxy)-1H-indol-2-yl)methyl)-3-methylurea;
1-methyl-3-((6-(thiazol-4-ylmethoxy)-1H-indol-2-yl)methyl)urea;
N-((5-chloro-6-((3-methylisoxazol-5-yl)methoxy)-1H-indol-2-yl)methyl)acetamide;
N-((5-chloro-6-(isoxazol-5-ylmethoxy)-1H-indol-2-yl)methyl)acetamide;
N-((5-fluoro-6-(isoxazol-5-ylmethoxy)-1H-indol-2-yl)methyl)acetamide;
N-((5-chloro-6-((2-methyl-2H-1,2,3-triazol-4-yl)methoxy)-1H-indol-2-yl)methyl)acetamide;
N-((5-fluoro-6-((2-methyl-2H-1,2,3-triazol-4-yl)methoxy)-1H-indol-2-yl)methyl)acetamide;
N-((5-fluoro-6-((3-methylisoxazol-5-yl)methoxy)-1H-indol-2-yl)methyl)acetamide;
N-((5-fluoro-6-(oxazol-4-ylmethoxy)-1H-indol-2-yl)methyl)acetamide;
N-((5-chloro-6-((6-fluoropyridin-2-yl)methoxy)-1H-indol-2-yl)methyl)acetamide;
N-((5-fluoro-6-((6-fluoropyridin-2-yl)methoxy)-1H-indol-2-yl)methyl)acetamide;
N-((5-chloro-6-((isoxazol-3-ylmethyl)amino)-1H-indol-2-yl)methyl)acetamide;
N-((5-fluoro-6-((isoxazol-3-ylmethyl)amino)-1H-indol-2-yl)methyl)acetamide;
N-((5-chloro-6-((thiazol-4-ylmethyl)amino)-1H-indol-2-yl)methyl)acetamide;
1-fluoro-N-((5-fluoro-6-(thiazol-4-ylmethoxy)-1H-indol-2-yl)methyl)cyclopropane-1-carboxamide;
N-((5-chloro-6-(thiazol-4-ylmethoxy)-1H-indol-2-yl)methyl)-1-fluorocyclopropane-1-carboxamide;
N-((5-chloro-6-(isoxazol-3-ylmethoxy)-1H-indol-2-yl)methyl)-1-fluorocyclopropane-1-carboxamide;
1-fluoro-N-((5-fluoro-6-(isoxazol-3-ylmethoxy)-1H-indol-2-yl)methyl)cyclopropane-1-carboxamide;
N-((5-methyl-6-(thiazol-4-ylmethoxy)-1H-indol-2-yl)methyl)acetamide;
N-((6-(isoxazol-3-ylmethoxy)-5-methyl-1H-indol-2-yl)methyl)acetamide; and
N-((5-fluoro-6-((2-methyloxazol-4-yl)methoxy)-1H-indol-2-yl)methyl)acetamide;
N-((5-chloro-6-((3-methylisoxazol-5-yl)methoxy)-1H-indol-2-yl)methyl)-2-(1-hydroxycyclopropyl)acetamide;
N-((5-chloro-6-((3-methylisoxazol-5-yl)methoxy)-1H-indol-2-yl)methyl)-2-cyclopropyl-2-hydroxyacetamide;
N-((5-fluoro-6-((3-methylisoxazol-5-yl)methoxy)-1H-indol-2-yl)methyl)-2-hydroxypropanamide;
N-((5-chloro-6-((3-methylisoxazol-5-yl)methoxy)-1H-indol-2-yl)methyl)-2-hydroxybutanamide;
N-((5-chloro-6-((3-methylisoxazol-5-yl)methoxy)-1H-indol-2-yl)methyl)-2-hydroxybutanamide;
N-((5-chloro-6-((3-methylisoxazol-5-yl)methoxy)-1H-indol-2-yl)methyl)-2-hydroxyacetamide;
N-((5-chloro-6-(thiazol-4-ylmethoxy)-1H-indol-2-yl)methyl)-2-hydroxypropanamide;
N-((5-chloro-6-(thiazol-4-ylmethoxy)-1H-indol-2-yl)methyl)-2-hydroxyacetamide;
N-((5-chloro-6-((3-methylisoxazol-5-yl)methoxy)-1H-indol-2-yl)methyl)-2-(oxetan-2-yl)acetamide;
N-((5-chloro-6-((3-methylisoxazol-5-yl)methoxy)-1H-indol-2-yl)methyl)-3-fluoro-2-hydroxypropanamide;
N-((5-chloro-6-((3-methylisoxazol-5-yl)methoxy)-1H-indol-2-yl)methyl)-2-hydroxycyclobutane-1-carboxamide;
N-((5-chloro-6-((3-methylisoxazol-5-yl)methoxy)-1H-indol-2-yl)methyl)-2-hydroxy-2-methylpropanamide;
N-((5-chloro-6-((3-methylisoxazol-5-yl)methoxy)-1H-indol-2-yl)methyl)-1-(hydroxymethyl)cyclopropane-1-carboxamide;
N-((5-chloro-6-((3-methylisoxazol-5-yl)methoxy)-1H-indol-2-yl)methyl)-1-hydroxycyclopropane-1-carboxamide;
N-((5-chloro-6-((3-methylisoxazol-5-yl)methoxy)-1H-indol-2-yl)methyl)-3-hydroxycyclobutane-1-carboxamide;
N-((5-chloro-6-(thiazol-4-ylmethoxy)-1H-indol-2-yl)methyl)-1-(hydroxymethyl)cyclopropane-1-carboxamide;
N-((5-fluoro-6-(thiazol-4-ylmethoxy)-1H-indol-2-yl)methyl)-1-(hydroxymethyl)cyclopropane-1-carboxamide;
N-((5-chloro-6-((3-methylisoxazol-5-yl)methoxy)-1H-indol-2-yl)methyl)-2-hydroxypropanamide;
N-((5-chloro-6-(thiazol-4-ylmethoxy)-1H-indol-2-yl)methyl)-2-hydroxypropanamide;
N-((5-chloro-6-((3-methylisoxazol-5-yl)methoxy)-1H-indol-2-yl)methyl)-2-(oxetan-3-yl)acetamide;
N-((5-chloro-6-(thiazol-4-ylmethoxy)-1H-indol-2-yl)methyl)oxetane-2-carboxamide;
N-((5-chloro-6-(thiazol-4-ylmethoxy)-1H-indol-2-yl)methyl)-2-methyloxetane-2-carboxamide;
N-((5-chloro-6-(thiazol-4-ylmethoxy)-1H-indol-2-yl)methyl)oxetane-3-carboxamide;
N-((5-fluoro-6-((3-methylisoxazol-5-yl)methoxy)-1H-indol-2-yl)methyl)oxetane-2-carboxamide;
N-((5-fluoro-6-((3-methylisoxazol-5-yl)methoxy)-1H-indol-2-yl)methyl)-2-methoxyacetamide;
N-((5-fluoro-6-(thiazol-4-ylmethoxy)-1H-indol-2-yl)methyl)oxetane-2-carboxamide;
N-((5-fluoro-6-(thiazol-4-ylmethoxy)-1H-indol-2-yl)methyl)-2-methoxyacetamide;
N-((5-chloro-6-(thiazol-4-ylmethoxy)-1H-indol-2-yl)methyl)tetrahydrofuran-2-carboxamide;
N-((5-chloro-6-(thiazol-4-ylmethoxy)-1H-indol-2-yl)methyl)tetrahydrofuran-3-carboxamide;
N-((5-chloro-6-(thiazol-4-ylmethoxy)-1H-indol-2-yl)methyl)tetrahydrofuran-2-carboxamide;
N-((5-chloro-6-(thiazol-4-ylmethoxy)-1H-indol-2-yl)methyl)oxetane-2-carboxamide;
N-((5-chloro-6-(thiazol-4-ylmethoxy)-1H-indol-2-yl)methyl)-2-methoxyacetamide;
N-((5-chloro-6-(thiazol-4-ylmethoxy)-1H-indol-2-yl)methyl)-2-methoxypropanamide;
N-((5-chloro-6-((3-methylisoxazol-5-yl)methoxy)-1H-indol-2-yl)methyl)-2-methoxyacetamide;
N-((5-chloro-6-((3-methylisoxazol-5-yl)methoxy)-1H-indol-2-yl)methyl)-3-methyloxetane-3-carboxamide;
N-((5-chloro-6-((3-methylisoxazol-5-yl)methoxy)-1H-indol-2-yl)methyl)oxetane-3-carboxamide;
N-((5-chloro-6-((3-methylisoxazol-5-yl)methoxy)-1H-indol-2-yl)methyl)-3-cyanooxetane-3-carboxamide;
N-((5-chloro-6-((3-methylisoxazol-5-yl)methoxy)-1H-indol-2-yl)methyl)oxetane-2-carboxamide;
N-((5-chloro-6-(thiazol-4-ylmethoxy)-1H-indol-2-yl)methyl)-3-methyloxetane-3-carboxamide;
N-((5-chloro-6-((3-methylisoxazol-5-yl)methoxy)-1H-indol-2-yl)methyl)oxetane-2-carboxamide;
N-((5-chloro-6-(thiazol-4-ylmethoxy)-1H-indol-2-yl)methyl)-1-(methoxymethyl)cyclopropane-1-carboxamide;
N-((5-fluoro-6-(thiazol-4-ylmethoxy)-1H-indol-2-yl)methyl)-1-(methoxymethyl)cyclopropane-1-carboxamide;
N-((5-fluoro-6-(thiazol-4-ylmethoxy)-1H-indol-2-yl)methyl)-2-hydroxypropanamide;
N-((5-fluoro-6-((3-methylisoxazol-5-yl)methoxy)-1H-indol-2-yl)methyl)-2-hydroxypropanamide;
N-((5-chloro-6-(thiazol-4-ylmethoxy)-1H-indol-2-yl)methyl)-2-hydroxypropanamide;
N-((5-chloro-6-((3-methylisoxazol-5-yl)methoxy)-1H-indol-2-yl)methyl)-3-hydroxybutanamide;
N-((5-chloro-6-((3-methylisoxazol-5-yl)methoxy)-1H-indol-2-yl)methyl)-2-(3-methyloxetan-3-yl)acetamide;
N-((5-chloro-6-((3-methylisoxazol-5-yl)methoxy)-1H-indol-2-yl)methyl)-2-(tetrahydrofuran-3-yl)acetamide;
N-((5-chloro-6-(thiazol-4-ylmethoxy)-1H-indol-2-yl)methyl)-2-methoxypropanamide;
N-((5-chloro-6-(thiazol-4-ylmethoxy)-1H-indol-2-yl)methyl)tetrahydrofuran-2-carboxamide;
N-((5-chloro-6-((3-methylisoxazol-5-yl)methoxy)-1H-indol-2-yl)methyl)-2-(difluoromethoxy)acetamide;
N-((5-chloro-6-((3-methylisoxazol-5-yl)methoxy)-1H-indol-2-yl)methyl)-2-(hydroxymethyl)tetrahydrofuran-2-carboxamide;
N-((5-chloro-6-((3-methylisoxazol-5-yl)methoxy)-1H-indol-2-yl)methyl)-7-oxabicyclo[2.2.1]heptane-1-carboxamide;
N-((5-chloro-6-((3-methylisoxazol-5-yl)methoxy)-1H-indol-2-yl)methyl)-7-oxabicyclo[2.2.1]heptane-2-carboxamide;
N-((5-chloro-6-((3-methylisoxazol-5-yl)methoxy)-1H-indol-2-yl)methyl)-7-oxabicyclo[2.2.1]heptane-2-carboxamide;
N-((5-chloro-6-((3-methylisoxazol-5-yl)methoxy)-1H-indol-2-yl)methyl)-2-oxabicyclo[2.1.1]hexane-4-carboxamide;
N-((5-chloro-6-((3-methylisoxazol-5-yl)methoxy)-1H-indol-2-yl)methyl)-2-(2-methoxyethoxy)acetamide;
N-((5-chloro-6-((3-methylisoxazol-5-yl)methoxy)-1H-indol-2-yl)methyl)-3-oxabicyclo[3.1.0]hexane-1-carboxamide;
N-((5-chloro-6-((3-methylisoxazol-5-yl)methoxy)-1H-indol-2-yl)methyl)-2-oxabicyclo[2.1.1]hexane-1-carboxamide;
N-((5-chloro-6-((3-methylisoxazol-5-yl)methoxy)-1H-indol-2-yl)methyl)-4-methylmorpholine-3-carboxamide;
N-((5-chloro-6-((3-methylisoxazol-5-yl)methoxy)-1H-indol-2-yl)methyl)-1,4-dioxane-2-carboxamide;
N-((5-chloro-6-((3-methylisoxazol-5-yl)methoxy)-1H-indol-2-yl)methyl)-3-fluorotetrahydrofuran-3-carboxamide;
N-((5-chloro-6-((3-methylisoxazol-5-yl)methoxy)-1H-indol-2-yl)methyl)-3,3-difluorotetrahydrofuran-2-carboxamide;
N-((5-chloro-6-((3-methylisoxazol-5-yl)methoxy)-1H-indol-2-yl)methyl)-2-methyltetrahydrofuran-2-carboxamide;
N-((5-chloro-6-(thiazol-4-ylmethoxy)-1H-indol-2-yl)methyl)tetrahydrofuran-3-carboxamide;
N-((5-chloro-6-(thiazol-4-ylmethoxy)-1H-indol-2-yl)methyl)-3-hydroxypropanamide;
N-((5-chloro-6-((3-methylisoxazol-5-yl)methoxy)-1H-indol-2-yl)methyl)-2-(tetrahydrofuran-2-yl)acetamide;
N-((5-chloro-6-((3-methylisoxazol-5-yl)methoxy)-1H-indol-2-yl)methyl)tetrahydrofuran-2-carboxamide;
N-((5-chloro-6-((3-methylisoxazol-5-yl)methoxy)-1H-indol-2-yl)methyl)-2-methoxypropanamide;
N-((5-chloro-6-(thiazol-4-ylmethoxy)-1H-indol-2-yl)methyl)oxetane-2-carboxamide;
N-((5-chloro-6-(thiazol-4-ylmethoxy)-1H-indol-2-yl)methyl)tetrahydrofuran-3-carboxamide;
N-({5-chloro-6-[(5-methyl-1,2,4-oxadiazol-3-yl)methoxy]-2-indolyl}methyl)1-methylcyclopropanecarboxamide;
N-({5-chloro-6-[(1,2,4-oxadiazol-3-yl)methoxy]-2-indolyl}methyl)1-methylcyclopropanecarboxamide;
N-({5-chloro-6-[(5-chloro-2-thienyl)methoxy]-2-indolyl}methyl)1-methylcyclopropanecarboxamide;
N-({5-chloro-6-[(2-chloro-3-thienyl)methoxy]-2-indolyl}methyl)1-methylcyclopropanecarboxamide;
N-({5-chloro-6-[(5-isothiazolyl)methoxy]-2-indolyl}methyl)1-methylcyclopropanecarboxamide;
N-({5-chloro-6-[(5-chloro-1,3-thiazol-2-yl)methoxy]-2-indolyl}methyl)1-methylcyclopropanecarboxamide;
N-({5-chloro-6-[(5-chloro-3-thienyl)methoxy]-2-indolyl}methyl)1-methylcyclopropanecarboxamide;
N-({5-chloro-6-[(4-chloro-5-isothiazolyl)methoxy]-2-indolyl}methyl)1-methylcyclopropanecarboxamide;
N-({5-chloro-6-[(5-isoxazolyl)methoxy]-2-indolyl}methyl)1-methylcyclopropanecarboxamide;
N-({5-fluoro-6-[(6-fluoro-2-pyridyl)methoxy]-2-indolyl}methyl)1-methylcyclopropanecarboxamide;
N-({5-fluoro-6-[(5-isoxazolyl)methoxy]-2-indolyl}methyl)1-methylcyclopropanecarboxamide;
N-({5-fluoro-6-[(5-methyl-3-isoxazolyl)methoxy]-2-indolyl}methyl)1-methylcyclopropanecarboxamide;
N-({5-fluoro-6-[(2-pyridyl)methoxy]-2-indolyl}methyl)1-methylcyclopropanecarboxamide;
N-({5-chloro-6-[2-(3-pyridyl)ethoxy]-2-indolyl}methyl)1-methylcyclopropanecarboxamide;
N-({5-fluoro-6-[(2-fluoro-3-pyridyl)methoxy]-2-indolyl}methyl)1-methylcyclopropanecarboxamide;
N-{[5-chloro-6-(2-methoxyethoxy)-2-indolyl]methyl}1-methylcyclopropanecarboxamide;
N-({5-chloro-6-[(3-pyridazinyl)methoxy]-2-indolyl}methyl)1-methylcyclopropanecarboxamide;
N-{[5-chloro-6-(2-cyclopropoxyethoxy)-2-indolyl]methyl}1-methylcyclopropanecarboxamide;
N-({5-chloro-6-[(3-oxetanyl)methoxy]-2-indolyl}methyl)1-methylcyclopropanecarboxamide;
N-({5-chloro-6-[(6-methoxy-3-pyridazinyl)methoxy]-2-indolyl}methyl)1-methylcyclopropanecarboxamide;
N-{[5-chloro-6-(2-trifluoromethoxyethoxy)-2-indolyl]methyl}acetamide;
N-({4-fluoro-6-[(3-methyl-5-isoxazolyl)methoxy]-2-indolyl}methyl)1-methylcyclopropanecarboxamide;
N-({4-fluoro-6-[(5-methyl-3-isoxazolyl)methoxy]-2-indolyl}methyl)1-methylcyclopropanecarboxamide;
N-({5-cyclopropyl-6-[(1,3-thiazol-4-yl)methoxy]-2-indolyl}methyl)1-methylcyclopropanecarboxamide;
N-({5-cyclopropyl-6-[(3-isoxazolyl)methoxy]-2-indolyl}methyl)1-methylcyclopropanecarboxamide;
N-({5,7-dichloro-6-[(5-methyl-3-isoxazolyl)methoxy]-2-indolyl}methyl)1-methylcyclopropanecarboxamide;
N-({5,7-dichloro-6-[(1,3-thiazol-4-yl)methoxy]-2-indolyl}methyl)1-methylcyclopropanecarboxamide;
N-({5-chloro-6-[(3-methyl-5-isoxazolyl)methoxy]-2-indolyl}(²H₂)methyl)acetamide;
N-({5,7-dichloro-6-[(3-isoxazolyl)methoxy]-2-indolyl}methyl)1-methylcyclopropanecarboxamide;
N-(1-{5-chloro-6-[(1,3-thiazol-4-yl)methoxy]-2-indolyl}ethyl)1-methylcyclopropanecarboxamide;
N-({5-(methylthio)-6-[(1,3-thiazol-4-yl)methoxy]-2-indolyl}methyl)1-methylcyclopropanecarboxamide;
N-({6-[(3-methyl-5-isoxazolyl)methoxy]-2-indolyl}methyl)acetamide;
N-({5-chloro-6-[(6-indolyl)methoxy]-2-indolyl}methyl)1-methylcyclopropanecarboxamide;
N-({5-chloro-6-[(2-chloro-4-fluoro-3-pyridyl)methoxy]-2-indolyl}methyl)1-methylcyclopropanecarboxamide;
N-[(5-chloro-6-{[5-(trifluoromethyl)-3-isoxazolyl]methoxy}-2-indolyl)methyl]1-methylcyclopropanecarboxamide;
N-({5-chloro-6-[(3,3-difluorocyclobutyl)methoxy]-2-indolyl}methyl)1-methylcyclopropanecarboxamide;
N-[(6-{[5-(trifluoromethyl)-3-isoxazolyl]methoxy}-2-indolyl)methyl]1-methylcyclopropanecarboxamide;
N-({5-chloro-6-[(3-oxetanyl)methoxy]-2-indolyl}methyl)acetamide;
N-({5-chloro-6-[(4-fluoro-3-pyridyl)methoxy]-2-indolyl}methyl)1-methylcyclopropanecarboxamide;
N-({5-fluoro-6-[(1,3-thiazol-4-yl)methoxy]-2-indolyl}methyl)-3-methyl-1-azetidinecarboxamide;
N-({5-fluoro-6-[(1,3-oxazol-4-yl)methoxy]-2-indolyl}methyl)-1-azetidinecarboxamide;
3-({5-chloro-6-[(1,3-thiazol-4-yl)methoxy]-2-indolyl}methyl)-1-cyclopropyl-1-methylurea;
N-({5-chloro-6-[(1,3-thiazol-4-yl)methoxy]-2-indolyl}methyl)-2-methyl-1-azetidinecarboxamide;
N-({5-chloro-6-[(3-isoxazolyl)methoxy]-2-indolyl}methyl)-2-methyl-1-azetidinecarboxamide;
N-({5-chloro-6-[(1,3-thiazol-4-yl)methoxy]-2-indolyl}methyl)-3-methoxy-1-azetidinecarboxamide;
N-({5-chloro-6-[(1,3-thiazol-4-yl)methoxy]-2-indolyl}methyl)-2-(hydroxymethyl)-1-azetidinecarboxamide;
N-({5-chloro-6-[(1,3-thiazol-4-yl)methoxy]-2-indolyl}methyl)-3-hydroxy-1-pyrrolidinecarboxamide;
N-({5-chloro-6-[(1,3-thiazol-4-yl)methoxy]-2-indolyl}methyl)-3-hydroxy-1-azetidinecarboxamide;
N-({5-chloro-6-[(1,3-thiazol-4-yl)methoxy]-2-indolyl}methyl)-3-(hydroxymethyl)-1-azetidinecarboxamide;
3-({5-chloro-6-[(3-methyl-5-isoxazolyl)methoxy]-2-indolyl}methyl)-1-ethylurea;
N-({5-chloro-6-[(3-methyl-5-isoxazolyl)methoxy]-2-indolyl}methyl)-3,3-difluoro-1-azetidinecarboxamide;
3-({5-chloro-6-[(3-methyl-5-isoxazolyl)methoxy]-2-indolyl}methyl)-1-(2-fluoroethyl)urea;
N-({5-chloro-6-[(3-methyl-5-isoxazolyl)methoxy]-2-indolyl}methyl)-3-fluoro-1-azetidinecarboxamide;
N-({5-chloro-6-[(3-methyl-5-isoxazolyl)methoxy]-2-indolyl}methyl)-(R)-3-fluoro-1-pyrrolidinecarboxamide;
3-({5-chloro-6-[(3-methyl-5-isoxazolyl)methoxy]-2-indolyl}methyl)-1-(2,2-difluoroethyl)urea;
N-({5-chloro-6-[(3-methyl-5-isoxazolyl)methoxy]-2-indolyl}methyl)-(S)-2-(difluoromethyl)-1-pyrrolidinecarboxamide;
N-({5-chloro-6-[(3-methyl-5-isoxazolyl)methoxy]-2-indolyl}methyl)-2-cyano-1-azetidinecarboxamide;
N-({5-chloro-6-[(3-methyl-5-isoxazolyl)methoxy]-2-indolyl}methyl)-3-(difluoromethyl)-1-pyrrolidinecarboxamide;
N-({5-chloro-6-[(3-methyl-5-isoxazolyl)methoxy]-2-indolyl}methyl)-6-oxa-1-aza-1-spiro[3.3]heptanecarboxamide;
3-({5-chloro-6-[(3-isoxazolyl)methoxy]-2-indolyl}methyl)-1-cyclopropyl-1-methylurea;
3-({5-chloro-6-[(1,3-thiazol-4-yl)methoxy]-2-indolyl}methyl)-1-(2-methoxyethyl)urea;
N-({5-fluoro-6-[(3-isoxazolyl)methoxy]-2-indolyl}methyl)-3-methyl-1-pyrrolidinecarboxamide;
N-({5-fluoro-6-[(3-isoxazolyl)methoxy]-2-indolyl}methyl)-2-methyl-1-pyrrolidinecarboxamide;
N-({5-fluoro-6-[(1,3-thiazol-4-yl)methoxy]-2-indolyl}methyl)-2-methyl-1-pyrrolidinecarboxamide;
N-({5-fluoro-6-[(1,3-thiazol-4-yl)methoxy]-2-indolyl}methyl)-3-methyl-1-pyrrolidinecarboxamide;
N-({5-fluoro-6-[(3-isoxazolyl)methoxy]-2-indolyl}methyl)-3-methyl-1-azetidinecarboxamide;
N-({5-chloro-6-[(3-isoxazolyl)methoxy]-2-indolyl}methyl)-3-methyl-1-azetidinecarboxamide;
N-({6-[(3-isoxazolyl)methoxy]-5-(methylthio)-2-indolyl}methyl)1-methylcyclopropanecarboxamide;
N-({6-[(1,7a-diaza-2-indenyl)methoxy]-5-chloro-2-indolyl}methyl)1-methylcyclopropanecarboxamide;
N-({5-chloro-6-[2-(3,3-difluorocyclobutyl)ethoxy]-2-indolyl}methyl)1-methylcyclopropanecarboxamide;
N-{[5-chloro-6-(2-cyclobutylethoxy)-2-indolyl]methyl}1-methylcyclopropanecarboxamide;
N-(1-{5-chloro-6-[(3-isoxazolyl)methoxy]-2-indolyl}ethyl)1-methylcyclopropanecarboxamide;
N-({5-chloro-6-[(1,4-dioxan-2-yl)methoxy]-2-indolyl}methyl)1-methylcyclopropanecarboxamide;
N-({5-chloro-6-[2-(tetrahydro-2-furyl)ethoxy]-2-indolyl}methyl)1-methylcyclopropanecarboxamide;
N-({5-chloro-6-[(3-methyl-5-isothiazolyl)methoxy]-2-indolyl}methyl)1-methylcyclopropanecarboxamide;
N-({5-chloro-6-[(5-methyl-3-isothiazolyl)methoxy]-2-indolyl}methyl)1-methylcyclopropanecarboxamide;
N-({5-chloro-6-[(5-chloro-1,3-thiazol-4-yl)methoxy]-2-indolyl}methyl)1-methylcyclopropanecarboxamide;
N-({5-fluoro-6-[(5-isoxazolyl)methoxy]-2-indolyl}methyl)-1-azetidinecarboxamide;
N-({5-chloro-6-[(6-fluoro-2-pyridyl)methoxy]-2-indolyl}methyl)-1-azetidinecarboxamide;
N-({5-chloro-6-[(1,3-oxazol-4-yl)methoxy]-2-indolyl}methyl)-1-azetidinecarboxamide;
N-({5-chloro-6-[(3-methyl-5-isoxazolyl)methoxy]-2-indolyl}methyl)-1-azetidinecarboxamide;
N-({5-chloro-6-[(2-methyl-2H-1,2,3-triazol-4-yl)methoxy]-2-indolyl}methyl)-1-azetidinecarboxamide;
N-({5-chloro-6-[(2-methyl-2H-1,2,3-triazol-4-yl)methoxy]-2-indolyl}methyl)-1-pyrrolidinecarboxamide;
N-[(5-chloro-6-{[(3-isoxazolyl)methyl]amino}-2-indolyl)methyl]-1-azetidinecarboxamide;
N-({5-chloro-6-[(2-methyl-1,3-oxazol-4-yl)methoxy]-2-indolyl}methyl)-1-azetidinecarboxamide;
N-({5-fluoro-6-[(2-methyl-2H-1,2,3-triazol-4-yl)methoxy]-2-indolyl}methyl)-1-azetidinecarboxamide;
N-({5-fluoro-6-[(2-methyl-2H-1,2,3-triazol-4-yl)methoxy]-2-indolyl}methyl)-1-pyrrolidinecarboxamide;
N-({5-chloro-6-[(2-methyl-1,3-oxazol-4-yl)methoxy]-2-indolyl}methyl)-1-pyrrolidinecarboxamide;
N-({5-chloro-6-[2-(3-isoxazolyl)ethyl]-2-indolyl}methyl)-1-pyrrolidinecarboxamide;
N-[(5-chloro-6-{[(3-isoxazolyl)methyl]amino}-2-indolyl)methyl]-1-pyrrolidinecarboxamide;
N-[(5-chloro-6-{[(1,3-thiazol-4-yl)methyl]amino}-2-indolyl)methyl]-1-pyrrolidinecarboxamide;
N-({6-[(1,3-thiazol-4-yl)methoxy]-5-trifluoromethoxy-2-indolyl}methyl)-1-pyrrolidinecarboxamide;
N-({6-[(3-isoxazolyl)methoxy]-5-trifluoromethoxy-2-indolyl}methyl)-1-azetidinecarboxamide;
N-({6-[(1,3-oxazol-4-yl)methoxy]-5-trifluoromethoxy-2-indolyl}methyl)-1-pyrrolidinecarboxamide;
N-({5-chloro-6-[2-(1,3-thiazol-4-yl)ethyl]-2-indolyl}methyl)-1-pyrrolidinecarboxamide;
N-({6-[2-(1,3-thiazol-4-yl)ethyl]-5-trifluoromethoxy-2-indolyl}methyl)-1-pyrrolidinecarboxamide;
N-({6-[(3-isoxazolyl)methoxy]-5-trifluoromethoxy-2-indolyl}methyl)-1-pyrrolidinecarboxamide;
N-({6-[(1,3-thiazol-4-yl)methoxy]-5-trifluoromethoxy-2-indolyl}methyl)-1-azetidinecarboxamide;
N-({4-fluoro-6-[(1,3-thiazol-4-yl)methoxy]-2-indolyl}methyl)-1-azetidinecarboxamide;
N-({4-fluoro-6-[(3-isoxazolyl)methoxy]-2-indolyl}methyl)-1-azetidinecarboxamide;
N-({5-chloro-3-ethyl-6-[(1,3-thiazol-4-yl)methoxy]-2-indolyl}methyl)-1-azetidinecarboxamide;
N-({5-chloro-6-[(6-fluoro-2-pyridyl)methoxy]-2-indolyl}methyl)-1-pyrrolidinecarboxamide;
N-({5-chloro-6-[(4-methyl-1,3-oxazol-2-yl)methoxy]-2-indolyl}methyl)-1-pyrrolidinecarboxamide;
N-({4-fluoro-6-[(3-methyl-5-isoxazolyl)methoxy]-2-indolyl}methyl)-1-azetidinecarboxamide;
N-({5-fluoro-6-[(1,3-oxazol-4-yl)methoxy]-2-indolyl}methyl)cyclopropanecarboxamide;
N-({5-chloro-6-[(1,3-oxazol-4-yl)methoxy]-2-indolyl}methyl)cyclopropanecarboxamide;
N-({5-fluoro-6-[(1,3-thiazol-4-yl)methoxy]-2-indolyl}methyl)cyclobutanecarboxamide;
N-({5-fluoro-6-[(3-isoxazolyl)methoxy]-2-indolyl}methyl)cyclobutanecarboxamide;
N-({5-chloro-6-[(1,3-thiazol-4-yl)methoxy]-2-indolyl}methyl)2,2-dimethylpropionamide;
N-({5-fluoro-6-[(2-methyl-2H-1,2,3-triazol-4-yl)methoxy]-2-indolyl}methyl)cyclopropanecarboxamide;
N-({6-[(1,3-thiazol-4-yl)methoxy]-5-trifluoromethoxy-2-indolyl}methyl)1-methylcyclopropanecarboxamide;
N-({5-chloro-6-[(1,3-thiazol-4-yl)methoxy]-2-indolyl}methyl)2,2-difluoropropionamide;
N-({5-fluoro-6-[(1,3-thiazol-4-yl)methoxy]-2-indolyl}methyl)2,2-difluoropropionamide;
N-({5-chloro-6-[(1,3-thiazol-4-yl)methoxy]-2-indolyl}methyl)cyanoacetamide;
N-({5-chloro-6-[(3-methyl-5-isoxazolyl)methoxy]-2-indolyl}methyl)propionamide;
N-({6-[(1,3-oxazol-4-yl)methoxy]-5-trifluoromethoxy-2-indolyl}methyl)1-methylcyclopropanecarboxamide;
N-({5-chloro-6-[(3-methyl-5-isoxazolyl)methoxy]-2-indolyl}methyl)2,2-difluoropropionamide;
N-({5-fluoro-6-[(3-methyl-5-isoxazolyl)methoxy]-2-indolyl}methyl)2,2-difluoropropionamide;
N-({5-chloro-6-[(3-methyl-5-isoxazolyl)methoxy]-2-indolyl}methyl)(S)-2-fluoropropionamide;
N-({5-chloro-6-[(1,3-thiazol-4-yl)methoxy]-2-indolyl}methyl)2-fluoropropionamide;
N-({5-chloro-6-[(1,3-thiazol-4-yl)methoxy]-2-indolyl}methyl)(S)-2-cyanopropionamide;
N-({5-chloro-6-[(3-methyl-5-isoxazolyl)methoxy]-2-indolyl}methyl)(S)-2-cyanopropionamide;
N-({5-chloro-6-[(3-methyl-5-isoxazolyl)methoxy]-2-indolyl}methyl)(R)-2-cyanopropionamide;
N-({5-chloro-6-[(3-methyl-5-isoxazolyl)methoxy]-2-indolyl}methyl)-2-azetidinecarboxamide;
N-({5-chloro-6-[(3-methyl-5-isoxazolyl)methoxy]-2-indolyl}methyl)cyclopropylacetamide;
N-({5-chloro-6-[(3-methyl-5-isoxazolyl)methoxy]-2-indolyl}methyl)-3-isoxazolecarboxamide;
N-[(5-chloro-6-{1[(5-methyl-3-isoxazolyl)methyl]amino}-2-indolyl)methyl]1-methylcyclopropanecarboxamide;
N-[(5-chloro-6-{1[(1,3-thiazol-4-yl)methyl]amino}-2-indolyl)methyl]1-methylcyclopropanecarboxamide;
N-({4-fluoro-6-[(3-isoxazolyl)methoxy]-2-indolyl}methyl)acetamide;
N-{[5-chloro-6-(2-methoxypropoxy)-2-indolyl]methyl}1-methylcyclopropanecarboxamide;
N-({5-chloro-6-[2-(tetrahydro-2-furyl)ethoxy]-2-indolyl}methyl)acetamide;
N-{[5-chloro-6-(2-cyclobutoxyethoxy)-2-indolyl]methyl}acetamide;
N-({5-chloro-6-[(1,4-dioxan-2-yl)methoxy]-2-indolyl}methyl)acetamide;
N-({5-chloro-6-[(2,2-difluorocyclopropyl)methoxy]-2-indolyl}methyl)acetamide;
N-{[5-chloro-6-(2-ethoxyethoxy)-2-indolyl]methyl}acetamide;
N-({6-[(1-benzofuran-6-yl)methoxy]-5-fluoro-2-indolyl}methyl)1-methylcyclopropanecarboxamide;
N-({6-[(1-benzofuran-6-yl)methoxy]-5-chloro-2-indolyl}methyl)1-methylcyclopropanecarboxamide;
N-{[5-chloro-6-(3-methoxycyclobutoxy)-2-indolyl]methyl}acetamide;
N-({5-chloro-6-[(tetrahydro-3-furyl)methoxy]-2-indolyl}methyl)acetamide;
N-({5-chloro-6-[(tetrahydro-2-furyl)methoxy]-2-indolyl}methyl)acetamide;
N-({6-[2-(1,3-oxazol-4-yl)ethyl]-5-trifluoromethoxy-2-indolyl}methyl)-1-azetidinecarboxamide;
N-({6-[2-(1,3-oxazol-4-yl)ethyl]-5-trifluoromethoxy-2-indolyl}methyl)-1-pyrrolidinecarboxamide;
N-[(5-chloro-6-{[(5-methyl-3-isoxazolyl)methyl]amino}-2-indolyl)methyl]-1-pyrrolidinecarboxamide;
N-[(5-fluoro-6-{[(3-isoxazolyl)methyl]amino}-2-indolyl)methyl]-1-azetidinecarboxamide;
N-[(5-fluoro-6-{[(3-isoxazolyl)methyl]amino}-2-indolyl)methyl]-1-pyrrolidinecarboxamide;
N-[(5-chloro-6-{[(1,3-thiazol-4-yl)methyl]amino}-2-indolyl)methyl]-1-azetidinecarboxamide;
N-({5-chloro-3-methyl-6-[(1,3-thiazol-4-yl)methoxy]-2-indolyl}methyl)-1-azetidinecarboxamide;
N-({3,5-dichloro-6-[(1,3-thiazol-4-yl)methoxy]-2-indolyl}methyl)-1-azetidinecarboxamide;
N-({3-chloro-5-methyl-6-[(1,3-thiazol-4-yl)methoxy]-2-indolyl}methyl)-1-azetidinecarboxamide;
N-({6-[(3-isoxazolyl)methoxy]-5-(trifluoromethyl)-2-indolyl}methyl)-1-pyrrolidinecarboxamide;
N-({6-[(3-isoxazolyl)methoxy]-5-(trifluoromethyl)-2-indolyl}methyl)-1-azetidinecarboxamide;
N-({6-[(1,3-thiazol-4-yl)methoxy]-5-(trifluoromethyl)-2-indolyl}methyl)-1-azetidinecarboxamide;
N-({6-[(1,3-thiazol-4-yl)methoxy]-5-(trifluoromethyl)-2-indolyl}methyl)-1-pyrrolidinecarboxamide;
N-({6-[(1,3-oxazol-4-yl)methoxy]-5-trifluoromethoxy-2-indolyl}methyl)-1-azetidinecarboxamide;
N-({5-fluoro-6-[(3-methyl-5-isoxazolyl)methoxy]-2-indolyl}methyl)-1-azetidinecarboxamide;
N-({5-fluoro-6-[(1,3-thiazol-4-yl)methoxy]-2-indolyl}methyl)1-[(²H₃)methyl]cyclopropanecarboxamide;
N-({5-chloro-6-[(1,3-thiazol-4-yl)methoxy]-2-indolyl}methyl)1-[(²H₃)methyl]cyclopropanecarboxamide;
N-({5-chloro-6-[(3-methyl-5-isoxazolyl)methoxy]-2-indolyl}methyl)1-cyanocyclopropanecarboxamide;
N-({5-chloro-6-[(3-methyl-5-isoxazolyl)methoxy]-2-indolyl}methyl)2-cyano-2-methylpropionamide;
N-({5-chloro-6-[(3-methyl-5-isoxazolyl)methoxy]-2-indolyl}methyl)1-cyanocyclobutanecarboxamide;
N-({5-chloro-6-[(3-isoxazolyl)methoxy]-2-indolyl}methyl)cyclobutanecarboxamide;
N-({5-chloro-6-[(1,3-thiazol-4-yl)methoxy]-2-indolyl}methyl)cyclopentanecarboxamide;
N-({5-chloro-6-[(1,3-thiazol-4-yl)methoxy]-2-indolyl}methyl)cyclobutanecarboxamide;
N-({5-chloro-6-[(3-isoxazolyl)methoxy]-2-indolyl}methyl)3,3-difluorocyclobutanecarboxamide;
N-({5-chloro-6-[(3-isoxazolyl)methoxy]-2-indolyl}methyl)cyclopentanecarboxamide;
N-({5-chloro-6-[(3-isoxazolyl)methoxy]-2-indolyl}methyl)2,2-dimethylpropionamide;
N-({5-chloro-6-[(6-fluoro-2-pyridyl)methoxy]-2-indolyl}methyl)propionamide;
N-({5-chloro-6-[(1,3-thiazol-4-yl)methoxy]-2-indolyl}methyl)3,3-difluorocyclobutanecarboxamide;
N-({5-fluoro-6-[(2-methyl-2H-1,2,3-triazol-4-yl)methoxy]-2-indolyl}methyl)1-methylcyclopropanecarboxamide;
N-({5-fluoro-6-[(6-fluoro-2-pyridyl)methoxy]-2-indolyl}methyl)cyclopropanecarboxamide;
N-({5-chloro-6-[(2-methyl-2H-1,2,3-triazol-4-yl)methoxy]-2-indolyl}methyl)cyclopropanecarboxamide;
N-({5-chloro-6-[(6-fluoro-2-pyridyl)methoxy]-2-indolyl}methyl)cyclopropanecarboxamide;
N-({5-chloro-6-[(1,3-oxazol-4-yl)methoxy]-2-indolyl}methyl)2-methylpropionamide;
N-({5-fluoro-6-[(1,3-oxazol-4-yl)methoxy]-2-indolyl}methyl)2-methylpropionamide;
N-({5-fluoro-6-[(3-isoxazolyl)methoxy]-2-indolyl}methyl)1-methylcyclobutanecarboxamide;
N-({5-chloro-6-[(2-pyridyl)methoxy]-2-indolyl}methyl)cyclopropanecarboxamide;
N-({5-chloro-6-[(3-isoxazolyl)methoxy]-2-indolyl}methyl)1-methylcyclobutanecarboxamide;
N-({5-fluoro-6-[(2-pyridyl)methoxy]-2-indolyl}methyl)cyclopropanecarboxamide;
N-({5-chloro-6-[(1,3-thiazol-4-yl)methoxy]-2-indolyl}methyl)1-methylcyclobutanecarboxamide;
N-({5-fluoro-6-[(1,3-thiazol-4-yl)methoxy]-2-indolyl}methyl)1-methylcyclobutanecarboxamide;
N-({6-[(3-isoxazolyl)methoxy]-5-trifluoromethoxy-2-indolyl}methyl)1-methylcyclopropanecarboxamide;
N-({6-[2-(1,3-thiazol-4-yl)ethyl]-5-trifluoromethoxy-2-indolyl}methyl)1-methylcyclopropanecarboxamide;
N-({6-[2-(3-isoxazolyl)ethyl]-5-trifluoromethoxy-2-indolyl}methyl)1-methylcyclopropanecarboxamide;
N-({5-chloro-6-[(1,3-thiazol-4-yl)methoxy]-2-indolyl}methyl)(1-azetidinyl)acetamide;
N-({5-chloro-6-[(1,3-thiazol-4-yl)methoxy]-2-indolyl}methyl)3,3-difluoropropionamide;
N-({6-[2-(1,3-oxazol-4-yl)ethyl]-5-trifluoromethoxy-2-indolyl}methyl)1-methylcyclopropanecarboxamide;
N-({4-fluoro-6-[(1,3-thiazol-4-yl)methoxy]-2-indolyl}methyl)propionamide;
N-({5-fluoro-6-[(1,3-thiazol-4-yl)methoxy]-2-indolyl}methyl)3,3-difluoropropionamide;
N-({4-fluoro-6-[(3-isoxazolyl)methoxy]-2-indolyl}methyl)propionamide;
N-({5-fluoro-6-[(3-methyl-5-isoxazolyl)methoxy]-2-indolyl}methyl)3,3-difluoropropionamide;
N-({5-chloro-6-[(3-methyl-5-isoxazolyl)methoxy]-2-indolyl}methyl)3,3-difluoropropionamide;
N-({5-chloro-6-[(3-methyl-5-isoxazolyl)methoxy]-2-indolyl}methyl)(1H-1,2,3-triazol-1-yl)acetamide;
N-({5-chloro-6-[(3-methyl-5-isoxazolyl)methoxy]-2-indolyl}methyl)(1-pyrazolyl)acetamide;
N-({5-chloro-6-[(3-methyl-5-isoxazolyl)methoxy]-2-indolyl}methyl)-5-isoxazolecarboxamide;
N-[(5-fluoro-6-{1[(1,3-thiazol-4-yl)methyl]amino}-2-indolyl)methyl]1-methylcyclopropanecarboxamide;
N-[(5-fluoro-6-{1[(3-isoxazolyl)methyl]amino}-2-indolyl)methyl]1-methylcyclopropanecarboxamide;
N-({4-fluoro-6-[(1,3-thiazol-4-yl)methoxy]-2-indolyl}methyl)acetamide;
N-({4,5-difluoro-6-[(1,3-thiazol-4-yl)methoxy]-2-indolyl}methyl)1-methylcyclopropanecarboxamide;
N-({4-fluoro-6-[(3-methyl-5-isoxazolyl)methoxy]-2-indolyl}methyl)acetamide;
N-({5-methyl-6-[(1,3-thiazol-4-yl)methoxy]-2-indolyl}methyl)-1-azetidinecarboxamide;
N-({5-methyl-6-[(1,3-thiazol-4-yl)methoxy]-2-indolyl}methyl)propionamide;
N-({6-[(3-isoxazolyl)methoxy]-5-methyl-2-indolyl}methyl)propionamide;
N-({5-methyl-6-[(1,3-thiazol-4-yl)methoxy]-2-indolyl}methyl)-1-pyrrolidinecarboxamide;
N-({6-[(3-isoxazolyl)methoxy]-5-methyl-2-indolyl}methyl)-1-azetidinecarboxamide;
N-({6-[(3-isoxazolyl)methoxy]-5-methyl-2-indolyl}methyl)-1-pyrrolidinecarboxamide;
N-({5-chloro-6-[(3-methyl-5-isoxazolyl)methoxy]-2-indolyl}methyl)2-hydroxycyclopentanecarboxamide;
N-({5-chloro-6-[(3-methyl-5-isoxazolyl)methoxy]-2-indolyl}methyl)3-hydroxy-2-methylpropionamide;
N-({5-chloro-6-[(3-methyl-5-isoxazolyl)methoxy]-2-indolyl}methyl)2-fluorocyclopropanecarboxamide;
N-({5-chloro-6-[(1,3-thiazol-4-yl)methoxy]-2-indolyl}methyl)2-fluorocyclopropanecarboxamide;
N-({5-chloro-6-[(3-methyl-5-isoxazolyl)methoxy]-2-indolyl}methyl)2-(3-oxetanyl)propionamide;
N-({5-chloro-6-[(1,3-thiazol-4-yl)methoxy]-2-indolyl}methyl)-1-methyl-2-oxo-3-azetidinecarboxamide;
N-({5-fluoro-6-[(2-methyl-1,3-oxazol-4-yl)methoxy]-2-indolyl}methyl)1-methylcyclopropanecarboxamide;
N-({5-fluoro-6-[(3-methyl-5-isoxazolyl)methoxy]-2-indolyl}methyl)propionamide;
N-({5-fluoro-6-[(2-methyl-1,3-oxazol-4-yl)methoxy]-2-indolyl}methyl)cyclopropanecarboxamide;
N-({5-fluoro-6-[(2-methyl-1,3-oxazol-4-yl)methoxy]-2-indolyl}methyl)propionamide;
N-({5-fluoro-6-[(2-methyl-1,3-oxazol-4-yl)methoxy]-2-indolyl}methyl)2-methylpropionamide;
N-({5-chloro-6-[(1,3-thiazol-4-yl)methoxy]-2-indolyl}methyl)-bicyclo[1.1.1]pentane-1-carboxamide;
N-({5-chloro-6-[(1,3-thiazol-4-yl)methoxy]-2-indolyl}methyl)-1-spiro[2.2]pentanecarboxamide;
N-({5-chloro-6-[(1,3-thiazol-4-yl)methoxy]-2-indolyl}methyl)3,3,3-trifluoropropionamide;
N-({5-chloro-6-[(1,3-thiazol-4-yl)methoxy]-2-indolyl}methyl)(bicyclo[1.1.1]pent-1-yl)acetamide;
N-({5-chloro-6-[(1,3-thiazol-4-yl)methoxy]-2-indolyl}methyl)-1-norbornanecarboxamide;
N-({5-chloro-6-[(1,3-thiazol-4-yl)methoxy]-2-indolyl}methyl)-2-spiro[3.3]heptanecarboxamide;
N-({5-chloro-6-[(1,3-thiazol-4-yl)methoxy]-2-indolyl}methyl)-1-spiro[2.3]hexanecarboxamide;
N-({5-chloro-6-[(1,3-thiazol-4-yl)methoxy]-2-indolyl}methyl)1-(difluoromethyl)cyclopropanecarboxamide;
N-({5-chloro-6-[(1,3-thiazol-4-yl)methoxy]-2-indolyl}methyl)difluoroacetamide;
N-({5-chloro-6-[(1,3-thiazol-4-yl)methoxy]-2-indolyl}methyl)3-methylcyclobutanecarboxamide;
N-({5-chloro-6-[(3-methyl-5-isoxazolyl)methoxy]-2-indolyl}methyl)(2,2,2-²H₃)acetamide;
N-({5-chloro-6-[(3-methyl-5-isoxazolyl)methoxy]-2-indolyl}methyl)difluoroacetamide;
N-({5-chloro-6-[(3-methyl-5-isoxazolyl)methoxy]-2-indolyl}methyl)3,3,3-trifluoropropionamide;
N-({5-chloro-6-[(3-methyl-5-isoxazolyl)methoxy]-2-indolyl}methyl)1-(difluoromethyl)cyclopropanecarboxamide;
N-({5-fluoro-6-[(1,3-thiazol-4-yl)methoxy]-2-indolyl}methyl)(2,2,2-²H₃)acetamide;
N-({5-fluoro-6-[(1,3-thiazol-4-yl)methoxy]-2-indolyl}methyl)difluoroacetamide;
N-({5-fluoro-6-[(1,3-thiazol-4-yl)methoxy]-2-indolyl}methyl)3,3,3-trifluoropropionamide;
N-({5-fluoro-6-[(1,3-thiazol-4-yl)methoxy]-2-indolyl}methyl)1-(difluoromethyl)cyclopropanecarboxamide;
N-({5-chloro-6-[(1,3-thiazol-4-yl)methoxy]-2-indolyl}methyl)(2,2,2-²H₃)acetamide;
N-({5-chloro-6-[(3-methyl-5-isoxazolyl)methoxy]-2-indolyl}methyl)1-fluorocyclopropanecarboxamide;
N-({5-fluoro-6-[(3-methyl-5-isoxazolyl)methoxy]-2-indolyl}methyl)1-fluorocyclopropanecarboxamide;
N-({5-chloro-6-[(3-methyl-5-isoxazolyl)methoxy]-2-indolyl}methyl)-1-methyl-2-oxo-3-pyrrolidinecarboxamide;
N-({5-chloro-6-[(3-methyl-5-isoxazolyl)methoxy]-2-indolyl}methyl)-1-methyl-2-azetidinecarboxamide;
N-({5-chloro-6-[(3-methyl-5-isoxazolyl)methoxy]-2-indolyl}methyl)-(S)-1-methyl-2-pyrrolidinecarboxamide;
N-({5-chloro-6-[(3-methyl-5-isoxazolyl)methoxy]-2-indolyl}methyl)-(R)-1-methyl-2-pyrrolidinecarboxamide;
N-({5-chloro-6-[(3-methyl-5-isoxazolyl)methoxy]-2-indolyl}methyl)(dimethylamino)acetamide;
N-({5-chloro-6-[(1,3-thiazol-4-yl)methoxy]-2-indolyl}methyl)(dimethylamino)acetamide;
N-({5-chloro-6-[(3-methyl-5-isoxazolyl)methoxy]-2-indolyl}methyl)-2-(difluoromethyl)-1-azetidinecarboxamide;
N-({5-chloro-6-[(3-methyl-5-isoxazolyl)methoxy]-2-indolyl}methyl)pyruvamide;
N-({5-chloro-6-[2-(1H-1,2,3-triazol-1-yl)ethyl]-2-indolyl}methyl)1-methylcyclopropanecarboxamide;
N-({5-chloro-6-[2-(2H-tetraazol-2-yl)ethyl]-2-indolyl}methyl)1-methylcyclopropanecarboxamide;
N-({5-chloro-6-[2-(1H-tetraazol-1-yl)ethyl]-2-indolyl}methyl)1-methylcyclopropanecarboxamide;
N-({5-chloro-6-[2-(2H-1,2,3-triazol-2-yl)ethyl]-2-indolyl}methyl)1-methylcyclopropanecarboxamide;
N-({5-methoxy-6-[2-(1-pyrazolyl)ethyl]-2-indolyl}methyl)1-methylcyclopropanecarboxamide;
N-({5-methoxy-6-[2-(1H-1,2,3-triazol-1-yl)ethyl]-2-indolyl}methyl)1-methylcyclopropanecarboxamide;
N-({5-chloro-6-[2-(1-imidazolyl)ethyl]-2-indolyl}methyl)1-methylcyclopropanecarboxamide;
N-({5-chloro-6-[2-(1-pyrazolyl)ethyl]-2-indolyl}methyl)1-methylcyclopropanecarboxamide;
N-({5-methoxy-6-[2-(2H-1,2,3-triazol-2-yl)ethyl]-2-indolyl}methyl)1-methylcyclopropanecarboxamide;
N-({5-fluoro-6-[2-(3-isoxazolyl)ethyl]-2-indolyl}methyl)1-methylcyclopropanecarboxamide;
N-({5-fluoro-6-[2-(5-methyl-3-isoxazolyl)ethyl]-2-indolyl}methyl)1-methylcyclopropanecarboxamide;
N-({5-fluoro-6-[2-(1,3-thiazol-4-yl)ethyl]-2-indolyl}methyl)1-methylcyclopropanecarboxamide;
N-({5-chloro-6-[2-(3-isoxazolyl)ethyl]-2-indolyl}methyl)-1-azetidinecarboxamide;
N-({6-[2-(1,3-thiazol-4-yl)ethyl]-5-trifluoromethoxy-2-indolyl}methyl)-1-azetidinecarboxamide;
N-({6-[2-(3-isoxazolyl)ethyl]-5-trifluoromethoxy-2-indolyl}methyl)-1-azetidinecarboxamide;
N-({6-[2-(3-isoxazolyl)ethyl]-5-trifluoromethoxy-2-indolyl}methyl)-1-pyrrolidinecarboxamide;
N-({5-chloro-6-[2-(1,3-thiazol-4-yl)ethyl]-2-indolyl}methyl)-1-azetidinecarboxamide;
N-({5-fluoro-6-[2-(1,3-thiazol-4-yl)ethyl]-2-indolyl}methyl)-1-azetidinecarboxamide;
N-({5-fluoro-6-[2-(1,3-thiazol-4-yl)ethyl]-2-indolyl}methyl)-1-pyrrolidinecarboxamide;
N-({5-fluoro-6-[2-(3-isoxazolyl)ethyl]-2-indolyl}methyl)-1-azetidinecarboxamide;
N-({5-fluoro-6-[2-(3-isoxazolyl)ethyl]-2-indolyl}methyl)-1-pyrrolidinecarboxamide;
N-({5-difluoromethoxy-6-[(1,3-thiazol-4-yl)methoxy]-2-indolyl}methyl)1-methylcyclopropanecarboxamide;
N-({5-difluoromethoxy-6-[(3-isoxazolyl)methoxy]-2-indolyl}methyl)1-methylcyclopropanecarboxamide;
N-({5-difluoromethoxy-6-[(3-methyl-5-isoxazolyl)methoxy]-2-indolyl}methyl)1-methylcyclopropanecarboxamide;
N-({5-chloro-6-[(2-indolyl)methoxy]-2-indolyl}methyl)1-methylcyclopropanecarboxamide;
N-({5-chloro-6-[(3-isoxazolylamino)methyl]-2-indolyl}methyl)1-methylcyclopropanecarboxamide;
N-({5-chloro-6-[(2-pyridylamino)methyl]-2-indolyl}methyl)1-methylcyclopropanecarboxamide;
N-({5-chloro-6-[(3-pyridylamino)methyl]-2-indolyl}methyl)1-methylcyclopropanecarboxamide;
N-({5-fluoro-6-[(3-isoxazolylamino)methyl]-2-indolyl}methyl)1-methylcyclopropanecarboxamide;
N-({5-fluoro-6-[(3-isoxazolyloxy)methyl]-2-indolyl}methyl)1-methylcyclopropanecarboxamide;
N-({5-chloro-6-[(3-isoxazolyloxy)methyl]-2-indolyl}methyl)1-methylcyclopropanecarboxamide;
N-({5-chloro-6-[(1,3-thiazol-4-yl)methoxy]-2-indolyl}methyl)(methylamino)acetamide;
N-({5-chloro-6-[(3-methyl-5-isoxazolyl)methoxy]-2-indolyl}methyl)(methylamino)acetamide;
N-({5-chloro-6-[(3-methyl-5-isoxazolyl)methoxy]-2-indolyl}methyl)aminoacetamide;
N-({5-chloro-6-[(3-methyl-5-isoxazolyl)methoxy]-2-indolyl}methyl)-(R)-2-pyrrolidinecarboxamide;
2-[3-({5-chloro-6-[(1,3-thiazol-4-yl)methoxy]-2-indolyl}methyl)ureido]ethanol;
ethyl ({5-chloro-6-[(1,3-thiazol-4-yl)methoxy]-2-indolyl}methyl)carbamate;
methyl ({5-fluoro-6-[(3-methyl-5-isoxazolyl)methoxy]-2-indolyl}methyl)carbamate;
methyl ({5-fluoro-6-[(1,3-thiazol-4-yl)methoxy]-2-indolyl}methyl)carbamate;
isopropyl ({5-chloro-6-[(1,3-thiazol-4-yl)methoxy]-2-indolyl}methyl)carbamate;
methyl ({5-chloro-6-[(1,3-thiazol-4-yl)methoxy]-2-indolyl}methyl)carbamate;
methyl ({5-chloro-6-[(3-methyl-5-isoxazolyl)methoxy]-2-indolyl}methyl)carbamate;
3-({5-chloro-6-[(1,3-thiazol-4-yl)methoxy]-2-indolyl}methyl)-1-methyl-1-[(4-pyrazolyl)methyl]urea;
3-({6-[(3-isoxazolyl)methoxy]-2-indolyl}methyl)-1-methyl-1-[(4-pyrazolyl)methyl]urea;
1-methyl-1-[(4-pyrazolyl)methyl]-3-({6-[(1,3-thiazol-4-yl)methoxy]-2-indolyl}methyl)urea;
3-({5-chloro-6-[(3-isoxazolyl)methoxy]-2-indolyl}methyl)-1-methyl-1-[(4-pyrazolyl)methyl]urea;
N-({6-[2-(3-isoxazolyl)ethyl]-5-methoxy-2-indolyl}methyl)1-methylcyclopropanecarboxamide;
N-({5-methoxy-6-[2-(1,3-thiazol-4-yl)ethyl]-2-indolyl}methyl)1-methylcyclopropanecarboxamide;
N-({6-[(1,3a-diaza-2-indenyl)methoxy]-5-chloro-2-indolyl}methyl)1-methylcyclopropanecarboxamide;
N-{[5-chloro-6-(cyclopropylmethoxy)-2-indolyl]methyl}1-methylcyclopropanecarboxamide;
N-{[5-chloro-6-(cyclobutylmethoxy)-2-indolyl]methyl}1-methylcyclopropanecarboxamide;
N-{[5-chloro-6-(2-cyclopropylethoxy)-2-indolyl]methyl}1-methylcyclopropanecarboxamide;
3-({5-chloro-6-[(3-methyl-5-isoxazolyl)methoxy]-2-indolyl}methyl)-1-cyclopropylurea;
1-({5-chloro-6-[(3-methyl-5-isoxazolyl)methoxy]-2-indolyl}methyl)urea;
3-({5-chloro-6-[(3-methyl-5-isoxazolyl)methoxy]-2-indolyl}methyl)-1-isopropylurea;
N-({5-chloro-6-[(1,3-thiazol-4-yl)methoxy]-2-indolyl}methyl)(S)-2,3-difluoropropionamide;
N-({6-[(1,3-thiazol-4-yl)methoxy]-3-(trifluoromethyl)-2-indolyl}methyl)1-methylcyclopropanecarboxamide;
N-({5-(2-cyanoethyl)-6-[(1,3-thiazol-4-yl)methoxy]-2-indolyl}methyl)1-methylcyclopropanecarboxamide;
N-({5-(2-cyanoethyl)-6-[(3-isoxazolyl)methoxy]-2-indolyl}methyl)1-methylcyclopropanecarboxamide;
N-({5-(cyanomethyl)-6-[(1,3-thiazol-4-yl)methoxy]-2-indolyl}methyl)1-methylcyclopropanecarboxamide;
N-({5-(cyanomethyl)-6-[(3-isoxazolyl)methoxy]-2-indolyl}methyl)1-methylcyclopropanecarboxamide;
N-({5-(difluoromethyl)-6-[(1,3-thiazol-4-yl)methoxy]-2-indolyl}methyl)1-methylcyclopropanecarboxamide;
N-({5-(difluoromethyl)-6-[(3-isoxazolyl)methoxy]-2-indolyl}methyl)1-methylcyclopropanecarboxamide;
N-({5-(difluoromethyl)-6-[(5-methyl-3-isoxazolyl)methoxy]-2-indolyl}methyl)1-methylcyclopropanecarboxamide;
N-({6-[(1,3-thiazol-4-yl)methoxy]-5-(2,2,2-trifluoroethyl)-2-indolyl}methyl)1-methylcyclopropanecarboxamide;
N-({6-[(3-isoxazolyl)methoxy]-5-(2,2,2-trifluoroethyl)-2-indolyl}methyl)1-methylcyclopropanecarboxamide;
N-({5-chloro-6-[2-(2,2-difluoroethoxy)ethoxy]-2-indolyl}methyl)acetamide;
N-({5-cyclopropyl-6-[(1,3-thiazol-4-yl)methoxy]-2-indolyl}methyl)-1-azetidinecarboxamide;
N-({5-cyclopropyl-6-[(1,3-thiazol-4-yl)methoxy]-2-indolyl}methyl)-1-pyrrolidinecarboxamide;
N-({5-cyclopropyl-6-[(3-isoxazolyl)methoxy]-2-indolyl}methyl)-1-azetidinecarboxamide;
N-({5-cyclopropyl-6-[(3-isoxazolyl)methoxy]-2-indolyl}methyl)-1-pyrrolidinecarboxamide;
N-({5-(1-fluorocyclopropyl)-6-[(3-isoxazolyl)methoxy]-2-indolyl}methyl)1-methylcyclopropanecarboxamide;
N-({5-(1-fluorocyclopropyl)-6-[(1,3-thiazol-4-yl)methoxy]-2-indolyl}methyl)1-methylcyclopropanecarboxamide;
N-({5-(2,2-difluorocyclopropyl)-6-[(1,3-thiazol-4-yl)methoxy]-2-indolyl}methyl)1-methylcyclopropanecarboxamide;
N-({5-(2,2-difluorocyclopropyl)-6-[(3-isoxazolyl)methoxy]-2-indolyl}methyl)1-methylcyclopropanecarboxamide;
N-({5-(3-isoxazolyl)-6-[(1,3-thiazol-4-yl)methoxy]-2-indolyl}methyl)1-methylcyclopropanecarboxamide;
N-({5-(5-isoxazolyl)-6-[(1,3-thiazol-4-yl)methoxy]-2-indolyl}methyl)1-methylcyclopropanecarboxamide;
N-({5-(1,3-oxazol-5-yl)-6-[(1,3-thiazol-4-yl)methoxy]-2-indolyl}methyl)1-methylcyclopropanecarboxamide;
N-({5-chloro-6-[(3-methyl-5-isoxazolyl)methoxy]-2-indolyl}methyl)trifluoroacetamide;
N-{[5-chloro-6-({5-[(²H₃)methyl]-3-isoxazolyl}methoxy)-2-indolyl]methyl}1-methylcyclopropanecarboxamide;
N-{[5-fluoro-6-({5-[(²H₃)methyl]-3-isoxazolyl}methoxy)-2-indolyl]methyl}1-methylcyclopropanecarboxamide;
N-({5-chloro-6-[(3-methyl-5-isoxazolyl)methoxy]-2-indolyl}methyl)-(2S,4R)-4-fluoro-2-pyrrolidinecarboxamide;
N-({5-chloro-6-[(3-methyl-5-isoxazolyl)methoxy]-2-indolyl}methyl)-(S)-4,4-difluoro-2-pyrrolidinecarboxamide;
N-({5-chloro-6-[(3-methyl-5-isoxazolyl)methoxy]-2-indolyl}methyl)-4-fluoro-1-methyl-2-pyrrolidinecarboxamide;
N-({5-chloro-6-[(3-methyl-5-isoxazolyl)methoxy]-2-indolyl}methyl)-(S)-4,4-difluoro-1-methyl-2-pyrrolidinecarboxamide; and
N-({5-chloro-6-[(1,3-thiazol-4-yl)methoxy]-2-indolyl}methyl)-1-methyl-2-azetidinecarboxamide.

### Methods of Treatment

Provided herein is a method of modulating SLC6A19 in a cell, comprising exposing the cell to (i) a composition comprising an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients. In some embodiments, modulating SLC6A19 comprises inhibition of SLC6A19.

Provided herein is a method of modulating SLC6A19 in a cell, comprising exposing the cell to (i) an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients. In some embodiments, modulating SLC6A19 comprises inhibition of SLC6A19.

Provided herein is a method of inhibiting SLC6A19 in a cell, comprising exposing the cell to (i) a composition comprising an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients.

Provided herein is a method of inhibiting SLC6A19 in a cell, comprising exposing the cell to (i) an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients.

Provided herein is a method of reducing systemic amino acid levels in an individual in need thereof, comprising administering to the individual (i) a composition comprising an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the amino acid is phenylalanine, tyrosine, glutamine, glycine, leucine, isoleucine, or valine. In some embodiments, the systemic phenylalanine, tyrosine, glutamine, glycine, leucine isoleucine, or valine levels in the individual is reduced upon treatment. In some embodiments, the phenylalanine, tyrosine, glutamine, glycine, leucine, isoleucine, or valine level is reduced at least 10%, at least 20%, at least 30% or at least 50% upon administration of the compound. In some embodiments, the amino acid is phenylalanine, tyrosine, glutamine, or glycine. In some embodiments, the systemic phenylalanine, tyrosine, glutamine, or glycine levels in the individual is reduced upon treatment. In some embodiments, the phenylalanine, tyrosine, glutamine, or glycine level is reduced at least 10%, at least 20%, at least 30% or at least 50% upon administration of the compound.

Provided herein is a method of reducing systemic amino acid levels in an individual in need thereof, comprising administering to the individual (i) an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients. In some embodiments, the amino acid is phenylalanine, tyrosine, glutamine, glycine, leucine, isoleucine, or valine. In some embodiments, the systemic phenylalanine, tyrosine, glutamine, glycine, leucine isoleucine, or valine levels in the individual is reduced upon treatment. In some embodiments, the phenylalanine, tyrosine, glutamine, glycine, leucine, isoleucine, or valine level is reduced at least 10%, at least 20%, at least 30% or at least 50% upon administration of the compound. In some embodiments, the amino acid is phenylalanine, tyrosine, glutamine, or glycine. In some embodiments, the systemic phenylalanine, tyrosine, glutamine, or glycine levels in the individual is reduced upon treatment. In some embodiments, the phenylalanine, tyrosine, glutamine, or glycine level is reduced at least 10%, at least 20%, at least 30% or at least 50% upon administration of the compound.

Provided herein is a method of treating a SLC6A19-mediated disease, disorder, or condition in an individual in need thereof, comprising administering to the individual (i) a composition comprising an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients. In some embodiments, the disease, disorder, or condition is responsive to SLC6A19 inhibition. In some embodiments, the SLC6A19-mediated disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders. In some embodiments, the SLC6A19-mediated disease, disorder, or condition is phenylketonuria (PKU), chronic kidney disease (CKD), metabolic syndrome, or metabolic diseases. In some embodiments, the SLC6A19-mediated disease, disorder, or condition is phenylketonuria (PKU). In some embodiments, the SLC6A19-mediated disease, disorder, or condition is chronic kidney disease (CKD). In some embodiments, the SLC6A19-mediated disease, disorder, or condition is a metabolic disease. In some embodiments, the SLC6A19-mediated disease, disorder, or condition is metabolic syndrome. In some embodiments, the SLC6A19-mediated disease, disorder, or condition is associated with abnormal levels of amino acids. In some embodiments, the SLC6A19-mediated disease, disorder, or condition is associated with a genetic defect in phenylalanine hydroxylase.

Provided herein is a method of treating a SLC6A19-mediated disease, disorder, or condition in an individual in need thereof, comprising administering to the individual (i) an effective amount of a compound of formula (I) or (I′), or any variation or embodiment thereof, or a stereoisomer or tautomer thereof, or a pharmaceutically acceptable salt of any of the foregoing, or (ii) a pharmaceutical composition, comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients. In some embodiments, the disease, disorder, or condition is responsive to SLC6A19 inhibition. In some embodiments, the SLC6A19-mediated disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders. In some embodiments, the SLC6A19-mediated disease, disorder, or condition is phenylketonuria (PKU), chronic kidney disease (CKD), metabolic syndrome, or metabolic diseases. In some embodiments, the SLC6A19-mediated disease, disorder, or condition is phenylketonuria (PKU). In some embodiments, the SLC6A19-mediated disease, disorder, or condition is chronic kidney disease (CKD). In some embodiments, the SLC6A19-mediated disease, disorder, or condition is a metabolic disease. In some embodiments, the SLC6A19-mediated disease, disorder, or condition is metabolic syndrome. In some embodiments, the SLC6A19-mediated disease, disorder, or condition is associated with abnormal levels of amino acids. In some embodiments, the SLC6A19-mediated disease, disorder, or condition is associated with a genetic defect in phenylalanine hydroxylase.

Provided herein is a method of treating a SLC6A19-mediated disease, disorder, or condition in an individual in need thereof, comprising administering to the individual (i) a compound of formula (I′), or any variation or embodiment thereof, or a stereoisomer or tautomer thereof, or a pharmaceutically acceptable salt of any of the foregoing, or (ii) a pharmaceutical composition comprising a compound of formula (I′), or any variation or embodiment thereof, or a stereoisomer or tautomer thereof, or a pharmaceutically acceptable salt of any of the foregoing, and one or more pharmaceutically acceptable excipients. In some embodiments, the disease, disorder, or condition is responsive to SLC6A19 inhibition. In some embodiments, the SLC6A19-mediated disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders. In some embodiments, the SLC6A19-mediated disease, disorder, or condition is phenylketonuria (PKU), chronic kidney disease (CKD), metabolic syndrome, or metabolic diseases. In some embodiments, the SLC6A19-mediated disease, disorder, or condition is phenylketonuria (PKU). In some embodiments, the SLC6A19-mediated disease, disorder, or condition is chronic kidney disease (CKD). In some embodiments, the SLC6A19-mediated disease, disorder, or condition is a metabolic disease. In some embodiments, the SLC6A19-mediated disease, disorder, or condition is metabolic syndrome. In some embodiments, the SLC6A19-mediated disease, disorder, or condition is associated with abnormal levels of amino acids. In some embodiments, the SLC6A19-mediated disease, disorder, or condition is associated with a genetic defect in phenylalanine hydroxylase. In some embodiments, the individual is a human.

In some embodiments, the compounds provided herein increase the lifespan of the individual. In some embodiments, the lifespan is increased at least 5, at least 10, or at least 20 years upon treatment.

In some embodiments, the compounds provided herein inhibit SLC6A19 at a concentration of less than 10 μM, less than 1 μM, less than 0.5 μM, less than 0.1 μM, less that 0.010 μM, or less that 0.001 μM. In some embodiments, the compounds provided herein inhibit SLC6A19 at a concentration of 1-10 μM, 0.01 to 1 μM, or 0.01 to 10 μM.

In some embodiments, the compounds have an IC₅₀ of less than 10 nM, less than 10 μM, less than 1 μM, less than 0.5 μM, or less than 0.1 μM. In some embodiments, the compounds provided herein have an IC₅₀ of 1 to 10 nM, 1 to 10 μM, 0.01 to 1 μM, 0.01 to 10 μM, 0.001 to 0.01 μM or 0.001 to 0.010 μM.

In some embodiments, the individual receiving treatment is a juvenile human or an infant. In some embodiments, the individual is less than 10 years old, less than 9 years old, less than 8 years old, less than 7 years old, less than 6 years old, less than 5 years old, less than 4 years old, less than 3 years old, less than 2 years old, or less than one year old. In some embodiments, the individual is a human.

In some embodiments, the individual has abnormal levels of amino acids. In some embodiments, the SLC6A19-mediated disease, disorder, or condition is associated with a genetic defect in phenylalanine hydroxylase. In some embodiments, the individual is a human.

In some embodiments of the foregoing, the administration is oral administration.

All methods described herein with reference to formula (I) or (I′), or a pharmaceutically acceptable salt of any of the foregoing, are also hereby described and embraced for any one of the other formulas detailed herein such as formula (II), the same as if each and every embodiment were specifically and individually listed. And, all methods described herein with reference to a compound of formula (I) or (I′), or a stereoisomer or tautomer thereof, or a pharmaceutically acceptable salt of any of the foregoing, such as embodiments related to m, n, R², R³, R⁵, R⁶, R⁷, R⁸, R⁹, R¹⁰, R^{11a}, R^{11b}, and X also apply to formula (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing

Provided herein is a compound of formula (II), or a stereoisomer or tautomer thereof, or a pharmaceutically acceptable salt of any of the foregoing, or a pharmaceutical composition comprising a compound of formula (II), for use in modulating SLC6A19 in a cell. In some embodiments, modulating SLC6A19 comprises inhibition of SLC6A19. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

Provided herein is a compound of formula (II), or a stereoisomer or tautomer thereof, or a pharmaceutically acceptable salt of any of the foregoing, or a pharmaceutical composition comprising a compound of formula (II), for use in inhibiting SLC6A19 in a cell. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B31), (I-B32), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (IF, (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

Provided herein is a compound of formula (II), or a stereoisomer or tautomer thereof, or a pharmaceutically acceptable salt of any of the foregoing, or a pharmaceutical composition comprising a compound of formula (II), for use in reducing systemic phenylalanine, tyrosine, glutamine, or glycine levels in an individual in need thereof. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B31), (I-B32), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

Provided herein is a compound of formula (II), or a stereoisomer or tautomer thereof, or a pharmaceutically acceptable salt of any of the foregoing, or a pharmaceutical composition comprising a compound of formula (II), for use in treating a SLC6A19-mediated disease, disorder, or condition in an individual in need thereof. In some embodiments, the disease, disorder, or condition is responsive to SLC6A19 inhibition. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

Provided herein is the use of a compound of formula (II), or a stereoisomer or tautomer thereof, or a pharmaceutically acceptable salt of any of the foregoing, or a pharmaceutical composition comprising a compound of formula (II), in the manufacture of a medicament for use in inhibiting SLC6A19 in a cell. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

Provided herein is the use of a compound of formula (II), or a stereoisomer or tautomer thereof, or a pharmaceutically acceptable salt of any of the foregoing, or a pharmaceutical composition comprising a compound of formula (II), in the manufacture of a medicament for use in reducing systemic phenylalanine, tyrosine, glutamine, or glycine levels in an individual in need thereof. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B31), (I-B32), (I-B33), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

Provided herein is the use of a compound of formula (II), or a stereoisomer or tautomer thereof, or a pharmaceutically acceptable salt of any of the foregoing, or a pharmaceutical composition comprising a compound of formula (II), in the manufacture of a medicament for use in treating a SLC6A19-mediated disease, disorder or condition in an individual in need thereof. In some embodiments, the disease, disorder, or condition is responsive to SLC6A19 inhibition. In some variations, the use also applies to any other applicable formula detailed herein, such as a compound of formula (I′), (I), (I-A), (I-A1), (I-A2), (I-A3), (I-A4), (I-A5), (I-A6), (I-B), (I-B1), (I-B2), (I-B3), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing, or any variation or embodiment thereof.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) a composition comprising an effective amount of a compound of formula (I) or (I′), or any variation or embodiment thereof, or a stereoisomer or tautomer thereof, or a pharmaceutically acceptable salt of any of the foregoing, and (ii) instructions for use in treating an SLC6A19-mediated disease, disorder, or condition in an individual in need thereof. Also provided herein are kits, comprising (i) a pharmaceutical composition comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients; and (ii) instructions for use in treating an SLC6A19-mediated disease, disorder, or condition in an individual in need thereof. In some embodiments, the individual is a human. In some embodiments, the disease, disorder, or condition is responsive to SLC6A19 inhibition.

Provided herein are kits, comprising (i) an effective amount of a compound of formula (I) or (I′), or any variation or embodiment thereof, or a stereoisomer or tautomer thereof, or a pharmaceutically acceptable salt of any of the foregoing, and (ii) instructions for use in treating an SLC6A19-mediated disease, disorder, or condition in an individual in need thereof. Also provided herein are kits, comprising (i) a pharmaceutical composition comprising an effective amount of a compound of formula (I) or (I′), or any variation or embodiment thereof, or a stereoisomer or tautomer thereof, or a pharmaceutically acceptable salt of any of the foregoing, and one or more pharmaceutically acceptable excipients; and (ii) instructions for use in treating an SLC6A19-mediated disease, disorder, or condition in an individual in need thereof. In some embodiments, the individual is a human. In some embodiments, the disease, disorder, or condition is responsive to SLC6A19 inhibition.

Provided herein are kits, comprising (i) a compound of formula (I′), or any variation or embodiment thereof, or a stereoisomer or tautomer thereof, or a pharmaceutically acceptable salt of any of the foregoing, and (ii) instructions for use in treating an SLC6A19-mediated disease, disorder, or condition in an individual in need thereof. Also provided herein are kits, comprising (i) a pharmaceutical composition comprising a compound of formula (I′), or any variation or embodiment thereof, or a stereoisomer or tautomer thereof, or a pharmaceutically acceptable salt of any of the foregoing, and one or more pharmaceutically acceptable excipients; and (ii) instructions for use in treating an SLC6A19-mediated disease, disorder, or condition in an individual in need thereof. In some embodiments, the individual is a human. In some embodiments, the disease, disorder, or condition is responsive to SLC6A19 inhibition.

Articles of manufacture are also provided, wherein the article of manufacture comprises a compound of formula (I) or (I′), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising a pharmaceutical composition comprising a compound of formula (I) or (I′), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

All kits, described herein with reference to formula (I) or (I′), or a pharmaceutically acceptable salt of any of the foregoing, are also hereby described and embraced for any one of the other formulas detailed herein such as formula (II), the same as if each and every embodiment were specifically and individually listed. And, all kits described herein with reference to a compound of formula (I) or (I′), or a stereoisomer or tautomer thereof, or a pharmaceutically acceptable salt of any of the foregoing, such as embodiments related to m, n, R², R³, R⁵, R⁶, R⁷, R⁸, R⁹, R¹⁰, R^{11a}, R^{11b}, and X also apply to formula (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

### Methods of Preparing

The present disclosure further provides processes for preparing the compounds of present invention. In some aspects, provided herein are processes of preparing a compound of (I′), (I), (I-A), (I-A1), (I-A2), (I-A3), (I-A4), (I-A5), (I-A6), (I-B), (I-B1), (I-B2), (I-B3), (I-B4), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), or (I-H), or a stereoisomer or tautomer thereof, or a pharmaceutically acceptable salt of any of the foregoing.

In some embodiments, a process for preparing a compound of formula (I) or (I′), or a stereoisomer or tautomer thereof, or a pharmaceutically acceptable salt of any of the foregoing, comprises:
(a) reacting a compound of formula (I′-1):

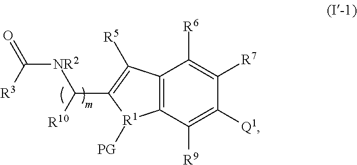

or a stereoisomer or tautomer thereof, or a pharmaceutically acceptable salt of any of the foregoing, wherein:
Q¹ is halo, boronic acid, or boronic ester;
PG is absent, or a protecting group;
m is an integer from 1 to 4;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
the C₁₋₆ alkyl of R⁵ and R⁷ are each independently optionally substituted with one or more halo or CN;
R⁶ and R⁹ are each independently H, or halo;
R¹⁰ is H; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (I′-2):

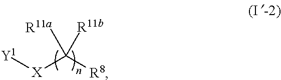

or a stereoisomer or tautomer thereof, or a pharmaceutically acceptable salt of any of the foregoing, wherein:
Y¹ is H, hydrazone, boronic acid, boronic ester, or halo;
n is an integer from 0 to 4;
X is —O—, —S—, —C(R^{12a})(R^{12b})—, —N(H)—, or —N(C₁₋₆ alkyl)-;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
in the presence of one or more coupling reagent, to provide a compound of formula (I′-3):

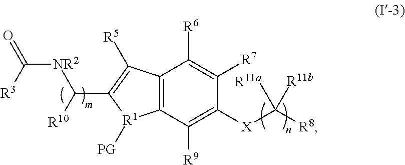

or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆ alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
   the C₁₋₆ alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
followed by,
(b) optionally contacting the compound of formula (I′-3) with a deprotecting agent;
to provide a compound of formula (I′).

In some embodiments, the protecting group is absent.

In some embodiments, the protecting group is an alkyl protecting group. In some embodiments, the protecting group is a tert-butoxy group. In some embodiments, the protecting group is an allyl protecting group. In some embodiments, the protecting group is a propenyl group. In some embodiments, the protecting group is an alkoxycarbonyl group. In some embodiments, the protecting group is a Boc group. In some embodiments, the protecting group is a sulfonyl group. In some embodiments, the protecting group is SO₂Ph.

In some embodiments, the compound of formula (I′-1) is a boronic acid, and the compound of formula (I′-2) is an alcohol. In some embodiments the reaction is an Ullman-type coupling. In some embodiments, the one or more coupling reagents comprises one or more reagents selected from the group consisting of a copper (II) acetate and pyridine.

In some embodiments, the compound of formula (I′-1) is an aryl halide and the compound of formula (I′-2) is an amine. In some embodiments, the one or more coupling reagents comprises one or more reagents selected from the group consisting of a palladium catalyst, a phosphine ligand, and a base. In some embodiments, the palladium catalyst is t-BuXPhos Pd-G3. In some embodiments, the phosphine ligand is t-BuXPhos. In some embodiments, the base is Cs₂CO₃.

In some embodiments, the compound of formula (I′-1) is a boronic acid, and the compound of formula (I′-2) is a hydrazone. In some embodiments the hydrazone is a tosyl hydrazone. In some embodiments, the one or more coupling reagents comprises a base. In some embodiments, the base is cesium carbonate.

In some embodiments, the compound of formula (I′-1) is an aryl halide, and the compound of formula (I′-2) is a boronic ester. In some embodiments, the compound of formula (I′-1) is a boronic ester, and the compound of formula (I′-2) is an aryl halide. In some embodiments the reaction is a Suzuki coupling. In some embodiments, the one or more coupling reagents comprises a catalyst. In some embodiments, the catalyst is Pd(dppf)Cl₂·CH₂Cl₂ or Pd(PPh₃)₄. In some embodiments, the one or more coupling reagents further comprises a base. In some embodiments, the base is potassium carbonate, or sodium carbonate. In some embodiments, the reaction further comprises a hydrogenation or cycloaddition to provide a compound of formula (I) or (I′).

In some embodiments, the deprotecting agent comprises an acid. In some embodiments the acid is HCl, TFA, or barbituric acid. In some embodiments, the deprotecting agent comprises tetrakis(triphenylphosphine)palladium(0). In some embodiments, the deprotecting agent comprises tetrakis(triphenylphosphine)palladium(0) and barbituric acid.

In some embodiments, a process for preparing a compound of formula (I) or (I′), or a stereoisomer or tautomer thereof, or a pharmaceutically acceptable salt of any of the foregoing, comprises:
reacting a compound of formula (II′-1):

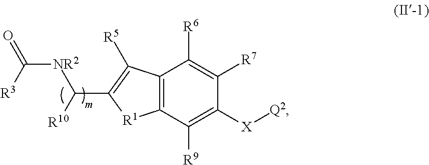

or a stereoisomer or tautomer thereof, or a pharmaceutically acceptable salt of any of the foregoing, wherein:
Q² is H, or (═O);
m is an integer from 1 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆ alkyl of R⁵ and R⁷ are each independently optionally substituted with one or more halo or CN;
R⁶ and R⁹ are each independently H, or halo;
R¹⁰ is H;
R^{12a} and R^{12b} are each independently, H, or C₁₋₆alkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (II′-2):

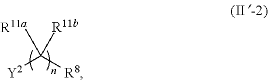

or a stereoisomer or tautomer thereof, or a pharmaceutically acceptable salt of any of the foregoing, wherein:
Y² is OH, (═O), halo, triphenylphosphonium salt, or sulfonic ester;
n is an integer from 0 to 4;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆ alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
in the presence of one or more coupling reagent, to provide a compound of formula (I′).

In some embodiments, the compound of formula (II-1) is an amine and the compound of formula (II-2) is an aldehyde or ketone. In some embodiments, the reaction is a reductive amination. In some embodiments, the one or more coupling reagents comprises a reducing reagent. In some embodiments, the reducing reagent is NaBH₃CN.

In some embodiments, the compounds of formula (II′-1) is a phenol, or thiol, and the compound of formula (II′-2) is an alkyl halide, or sulfonic ester. In some embodiments, the sulfonic ester is a mesylate or tosylate. In some embodiments, the reaction is an alkylation. In some embodiments, the one or more coupling reagents comprises an inorganic base. In some embodiments, the inorganic base is K₂CO₃ or Cs₂CO₃.

In some embodiments, the compound of formula (II′-1) is a phenol, and the compound of formula (II′-2) is an alcohol. In some embodiments, the reaction is a Mitsunobu reaction. In some embodiments, the one or more coupling reagents comprises a trialkylphosphine. In some embodiments, the trialkylphosphine is PBu₃. In some embodiments, the one or more coupling reagents further comprises an azodicarboxylate ester. In some embodiments, the azodicarboxylate ester is tetramethylazodicarboxamide.

In some embodiments, the compound of formula (II′-1) is an aldehyde or ketone, and the compound of formula (II′-2) is a triphenylphosphonium salt. In some embodiments the reaction is a Wittig reaction. In some embodiments, the one or more coupling reagents comprises a base. In some embodiments, the base is NaH.

In some embodiments, the compound of formula (II′-1) is a phenol, and the compound of formula (II′-2) is a sulfonic ester. In some embodiments, the sulfonic ester is a mesylate of tosylate.

In some embodiments, the one or more coupling reagents comprises an inorganic base. In some embodiments, the inorganic base is cesium carbonate. In some embodiments, the inorganic base is K₂CO₃ or Cs₂CO₃.

In some embodiments, the compound of formula (II′-1) is an aldehyde or ketone, and the compound of formula (II′-2) is a triphenylphosphonium salt or ylide. In some embodiments, the reaction further comprises a hydrogenation or cycloaddition step to provide a compound of formula (I) or (I′).

In some embodiments, the compounds of formula (II′-1) is an amine, and the compound of formula (II-2) is an alkyl halide. In some embodiments, the reaction is an alkylation. In some embodiments, the one or more coupling reagents comprises an organic base. In some embodiments, the organic base is DIEA.

In some embodiments, the one or more reaction further comprises a hydrogenation step or cyclopropanation step to derive a compound formula (I) or (I′).

In some embodiments, a process for preparing a compound of formula (I) or (I′), or a stereoisomer or tautomer thereof, or a pharmaceutically acceptable salt of any of the foregoing, comprises:
reacting a compound of formula (III′-1):

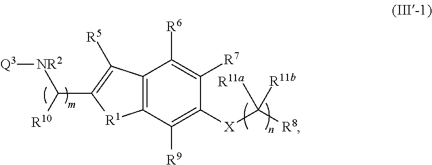

or a stereoisomer or tautomer thereof, or a pharmaceutically acceptable salt of any of the foregoing, wherein:
Q³ is H;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
the C₁₋₆ alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (III′-2):

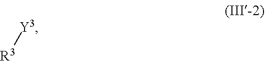

or a stereoisomer or tautomer thereof, or a pharmaceutically acceptable salt of any of the foregoing, wherein:
Y³ is —C(O)OH, or —C(O)-halo;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a}; and
each R^{a} is, independently at each occurrence, —OH, halo, C₁₋₆alkyl, C₁₋₆haloalkyl, or C₃₋₁₀cycloalkyl;
in the presence of one or more coupling reagent, to provide a compound of formula (I′).

In some embodiments, the compound of formula (III-1) is an amine, and the compound of formula (III-2) is a carboxylic acid. In some embodiments, the one or more coupling reagents comprises HATU. In some embodiments, the one or more coupling reagents further comprises a base. In some embodiments, the base is a tertiary amine. In some embodiments the base is DIEA. In some embodiments, the one or more coupling reagents comprises EDCI, HOBt and a tertiary amine base such as DIEA.

In some embodiments, the compound of formula (III′-1) is an amine, and the compound of formula (III′-2) is an acyl halide. In some embodiments, the one or more coupling reagents comprises a base. In some embodiments, the one or more coupling reagents comprises an organic base. In some embodiments, the organic base is a tertiary amine. In some embodiments the tertiary amine is DIEA.

In some embodiments, a process for preparing a compound of formula (I′), or a stereoisomer or tautomer thereof, or a pharmaceutically acceptable salt of any of the foregoing, comprises:
(a) reacting a compound of formula (IV′-1):

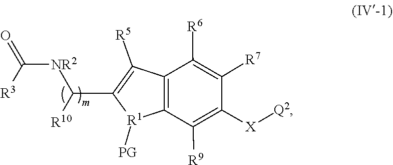

or a stereoisomer or tautomer thereof, or a pharmaceutically acceptable salt of any of the foregoing, wherein:
PG is absent or a protecting group;
Q² is H, or (═O);
m is an integer from 1 to 4;
X is —O—, —S—, —C(R^{12a})(R^{12b})—, —N(H)—, or —N(C₁₋₆ alkyl)-;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
   the C₁₋₆ alkyl of R⁵ and R⁷ are each independently optionally substituted with one or more halo or CN;
R⁶ and R⁹ are each independently H, or halo;
R¹⁰ is H;
R^{12a} and R^{12b} are each independently, H, or C₁₋₆alkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (II′-2):

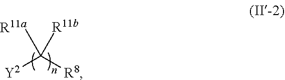

or a stereoisomer or tautomer thereof, or a pharmaceutically acceptable salt of any of the foregoing, wherein:
Y² is OH, (═O), halo, triphenylphosphonium salt, or sulfonic ester;
n is an integer from 0 to 4; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
in the presence of one or more coupling reagents, to provide a compound of formula (I′-3):

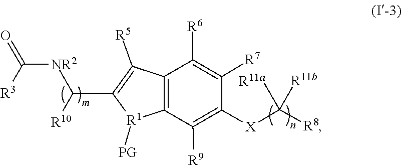

or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆ alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
the C₁₋₆ alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
followed by,
(b) optionally contacting the compound of formula (I′-3) with a deprotecting agent;
to provide a compound of formula (I′).

In some embodiments, the compound of formula (II′-1) is an amine and the compound of formula (II′-2) is an aldehyde or ketone. In some embodiments, the reaction is a reductive amination. In some embodiments, the one or more coupling reagents comprises a reducing reagent. In some embodiments, the reducing reagent is NaBH₃CN.

In some embodiments, the compounds of formula (II′-1) is a phenol, or thiol, and the compound of formula (II′-2) is an alkyl halide, or sulfonic ester. In some embodiments, the sulfonic ester is a mesylate or tosylate. In some embodiments, the reaction is an alkylation. In some embodiments, the one or more coupling reagents comprises an inorganic base. In some embodiments, the inorganic base is K₂CO₃ or Cs₂CO₃.

In some embodiments, the protecting group is absent.

In some embodiments, the protecting group is an alkyl protecting group. In some embodiments, the protecting group is a tert-butoxy group. In some embodiments, the protecting group is an allyl protecting group. In some embodiments, the protecting group is a propenyl group. In some embodiments, the protecting group is an alkoxycarbonyl group. In some embodiments, the protecting group is a Boc group. In some embodiments, the protecting group is a sulfonyl group. In some embodiments, the protecting group is SO₂Ph. In some embodiments, the protecting group is an alkyl sulfonate. In some embodiments, the protecting group is a mesylate or tosylate.

In some embodiments, the compound of formula (IV′-1) is a phenol, and the compound of formula (II′-2) is an alcohol. In some embodiments, the reaction is a Mitsunobu reaction. In some embodiments, the one or more coupling reagents comprises a trialkylphosphine. In some embodiments, the trialkylphosphine is PBu₃. In some embodiments, the one or more coupling reagents further comprises an azodicarboxylate ester. In some embodiments, the azodicarboxylate ester is tetramethylazodicarboxamide.

In some embodiments, the compound of formula (IV′-1) is an aldehyde or ketone, and the compound of formula (II′-2) is a triphenylphosphonium salt. In some embodiments the reaction is a Wittig reaction. In some embodiments, the one or more coupling reagents comprises a base. In some embodiments, the base is NaH.

In some embodiments, the compound of formula (IV′-1) is a phenol, and the compound of formula (II′-2) is a sulfonic ester. In some embodiments, the sulfonic ester is a mesylate of tosylate. In some embodiments, the one or more coupling reagents comprises an inorganic base. In some embodiments, the inorganic base is cesium carbonate. In some embodiments, the inorganic base is K₂CO₃ or Cs₂CO₃.

In some embodiments, the compound of formula (IV′-1) is an aldehyde or ketone, and the compound of formula (II′-2) is a triphenylphosphonium salt or ylide. In some embodiments, the reaction further comprises a hydrogenation or cycloaddition step to provide a compound of formula (I) or (I′).

In some embodiments, the compounds of formula (IV′-1) is an amine, and the compound of formula (II-2) is an alkyl halide. In some embodiments, the reaction is an alkylation. In some embodiments, the one or more coupling reagents comprises an organic base. In some embodiments, the organic base is DIEA.

In some embodiments, the deprotecting agent comprises an acid. In some embodiments the acid is HCl, TFA, or barbituric acid. In some embodiments, the deprotecting agent comprises tetrakis(triphenylphosphine)palladium(0). In some embodiments, the deprotecting agent comprises tetrakis(triphenylphosphine)palladium(0) and barbituric acid. In some embodiments, the deprotecting agent comprises Mg.

In some embodiments, the one or more reaction further comprises a hydrogenation step or cyclopropanation step to derive a compound formula (I) or (I′).

All methods, described herein with reference to formula (I) or (I′), or a pharmaceutically acceptable salt of any of the foregoing, are also hereby described and embraced for any one of the other formulas detailed herein such as formula (II), the same as if each and every embodiment were specifically and individually listed. And, all methods described herein with reference to formula (I) or (I′), or a stereoisomer or tautomer thereof, or a pharmaceutically acceptable salt of any of the foregoing, such as embodiments related to m, n, R², R³, R⁵, R⁶, R⁷, R⁸, R⁹, R¹⁰, R^{11a}, R^{11b}, and X also apply to formula (I-A), (I-A1), (I-A2), (I-A3), (I-A4), (I-A5), (I-A6), (I-B), (I-B1), (I-B2), (I-B3), (I-B34), (I-C), (I-C1), (I-C2), (I-C3), (I-C4), (I-C5), (I-D), (I-D1), (I-D2), (I-D3), (I-E), (I-E1), (I-E2), (I-E3), (I-F), (I-G), (I-H), (II-A), (II-B), (II-C), (II-D), (II-D1), (II-D2), (II-E), (II-G), (II-H), or a stereoisomer or tautomer thereof, or a pharmaceutically acceptable salt of any of the foregoing.

### Enumerated Embodiments

Embodiment 1. A compound of formula (I):

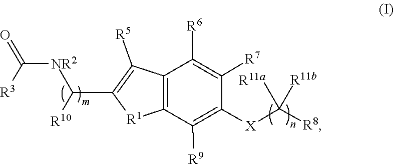

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H; R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl.

Embodiment 2. The compound of embodiment 1, or a stereoisomer or tautomer thereof, or a pharmaceutically acceptable salt of any of the foregoing, wherein the compound is a compound of formula (I-A):

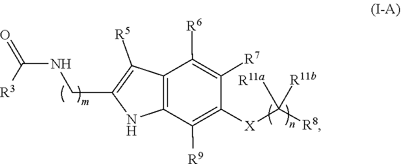

Embodiment 3. The compound of embodiment 1, or embodiment 2, or a stereoisomer or tautomer thereof, or a pharmaceutically acceptable salt of any of the foregoing, wherein m is an integer from 1-2.

Embodiment 4. The compound of any one of embodiments 1-3, or a stereoisomer or tautomer thereof, or a pharmaceutically acceptable salt of any of the foregoing, wherein m is 1.

Embodiment 5. The compound of any one of embodiments 1-4, or a stereoisomer or tautomer thereof, or a pharmaceutically acceptable salt of any of the foregoing, wherein n is an integer from 0-2.

Embodiment 6. The compound of any one of embodiments 1-5, or a stereoisomer or tautomer thereof, or a pharmaceutically acceptable salt of any of the foregoing, wherein n is 1.

Embodiment 7. The compound of any one of embodiments 1-5, or a stereoisomer or tautomer thereof, or a pharmaceutically acceptable salt of any of the foregoing, wherein n is 0.

Embodiment 8. The compound of any one of embodiments 1-6, or a stereoisomer or tautomer thereof, or a pharmaceutically acceptable salt of any of the foregoing, wherein m is 1 and n is 1.

Embodiment 9. The compound of any one of embodiments 1-5, or a stereoisomer or tautomer thereof, or a pharmaceutically acceptable salt of any of the foregoing, wherein m is 1 and n is 1.

Embodiment 10. The compound of any one of embodiments 1-9, or a stereoisomer or tautomer thereof, or a pharmaceutically acceptable salt of any of the foregoing, wherein R³ is C₁₋₃ alkyl, C₁₋₃haloalkyl, C₃₋₆cycloalkyl, —N(R⁴)₂, or 4-6 membered heterocyclyl, wherein each R³ is optionally substituted with one or more R^{a}.

Embodiment 11. The compound of any one of embodiments 1-10, or a stereoisomer or tautomer thereof, or a pharmaceutically acceptable salt of any of the foregoing, wherein R³ is C₁₋₃alkyl.

Embodiment 12. The compound of any one of embodiments 1-11, or a stereoisomer or tautomer thereof, or a pharmaceutically acceptable salt of any of the foregoing, wherein R³ is methyl, ethyl, or isopropyl.

Embodiment 13. The compound of any one of embodiments 1-10, or a stereoisomer or tautomer thereof, or a pharmaceutically acceptable salt of any of the foregoing, wherein R³ is C₃₋₆cycloalkyl optionally substituted with one or more R^{a}.

Embodiment 14. The compound of any one of embodiments 1-10, or 13, or a stereoisomer or tautomer thereof, or a pharmaceutically acceptable salt of any of the foregoing, wherein R³ is C₃₋₆cycloalkyl optionally substituted with one or more C₁₋₃alkyl, or C₁₋₃haloalkyl.

Embodiment 15. The compound of any one of embodiments 1-10, 13, or 14 or a stereoisomer or tautomer thereof, or a pharmaceutically acceptable salt of any of the foregoing, wherein R³ is selected from the group consisting of

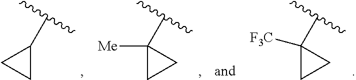

Embodiment 16. The compound of any one of embodiments 1-10, or a stereoisomer or tautomer thereof, or a pharmaceutically acceptable salt of any of the foregoing, wherein R³ is —N(R⁴)₂, and each R⁴ is independently H, C₁₋₃alkyl, C₁₋₃haloalkyl, or C₃₋₆cycloalkyl.

Embodiment 17. The compound of any one of embodiments 1-10, or 16, or a stereoisomer or tautomer thereof, or a pharmaceutically acceptable salt of any of the foregoing, wherein R³ is —N(R⁴)₂, and each R⁴ is independently H, or C₁₋₃alkyl.

Embodiment 18. The compound of any one of embodiments 1-10, or a stereoisomer or tautomer thereof, or a pharmaceutically acceptable salt of any of the foregoing, wherein R³ is 4-6 membered heterocyclyl, optionally substituted with one or more R^{a}.

Embodiment 19. The compound of any one of embodiments 1-10, or 18, or a stereoisomer or tautomer thereof, or a pharmaceutically acceptable salt of any of the foregoing, wherein R³ is —N(R⁴)₂, and both R⁴ are taken together with the N atom to which they are attached to form a 4-6 membered heterocyclyl optionally substituted with one or more R^{a}.

Embodiment 20. The compound of any one of embodiments 1-10, 18, or 19, or a stereoisomer or tautomer thereof, or a pharmaceutically acceptable salt of any of the foregoing, wherein R³ is —N(R⁴)₂, and both R⁴ are taken together with the N atom to which they are attached to form a 4-6 membered heterocyclyl optionally substituted with one or more halo, C₁₋₃alkyl, or C₁₋₃haloalkyl.

Embodiment 21. The compound of any one of embodiments 1-10, or 18-20, or a stereoisomer or tautomer thereof, or a pharmaceutically acceptable salt of any of the foregoing, wherein R³ is selected from the group consisting of

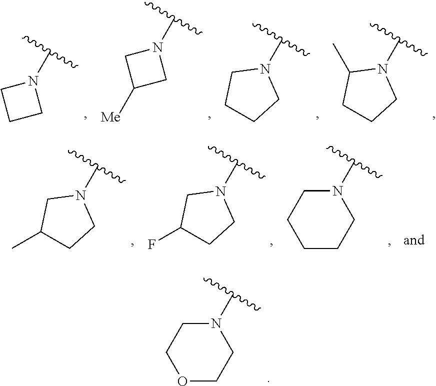

Embodiment 22. The compound of any one of embodiments 1-21, or a stereoisomer or tautomer thereof, or a pharmaceutically acceptable salt of any of the foregoing, wherein R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN.

Embodiment 23. The compound of any one of embodiments 1-22, or a stereoisomer or tautomer thereof, or a pharmaceutically acceptable salt of any of the foregoing, wherein R⁵ and R⁷ are each independently H, halo, C₁₋₃alkyl, or C₁₋₃haloalkyl.

Embodiment 24. The compound of any one of embodiments 1-23, or a stereoisomer or tautomer thereof, or a pharmaceutically acceptable salt of any of the foregoing, wherein R⁵ and R⁷ are each independently H.

Embodiment 25. The compound of any one of embodiments 1-23, or a stereoisomer or tautomer thereof, or a pharmaceutically acceptable salt of any of the foregoing, wherein one of R⁵ and R⁷ is H, and the other of R⁵ and R⁷ is independently halo, C₁₋₃alkyl, or C₁₋₃haloalkyl.

Embodiment 26. The compound of any one of embodiments 1-25, or a stereoisomer or tautomer thereof, or a pharmaceutically acceptable salt of any of the foregoing, wherein R⁶ and R⁹ are each independently H, or halo.

Embodiment 27. The compound of any one of embodiments 1-26, or a stereoisomer or tautomer thereof, or a pharmaceutically acceptable salt of any of the foregoing, wherein R⁶ and R⁹ are each independently H, F, or Cl.

Embodiment 28. The compound of any one of embodiments 1-27, or a stereoisomer or tautomer thereof, or a pharmaceutically acceptable salt of any of the foregoing, wherein R⁶ and R⁹ are each independently H.

Embodiment 29. The compound of any one of embodiments 1-27, or a stereoisomer or tautomer thereof, or a pharmaceutically acceptable salt of any of the foregoing, wherein one of R⁶ and R⁹ is H, and the other of R⁶ and R⁹ is F, or Cl.

Embodiment 30. The compound of any one of embodiments 1-29, or a stereoisomer or tautomer thereof, or a pharmaceutically acceptable salt of any of the foregoing, wherein R⁸ is —C(O)N(R⁴)₂, C₃₋₆cycloalkyl, C₆₋₁₂aryl, 3-6 membered heterocyclyl, or 5-10 membered heteroaryl, wherein each R⁸ is optionally substituted with one or more R^{a}.

Embodiment 31. The compound of any one of embodiments 1-30, or a stereoisomer or tautomer thereof, or a pharmaceutically acceptable salt of any of the foregoing, wherein R⁸ is —C(O)N(R⁴)₂.

Embodiment 32. The compound of any one of embodiments 1-31, or a stereoisomer or tautomer thereof, or a pharmaceutically acceptable salt of any of the foregoing, wherein R⁸ is —C(O)N(R⁴)₂, and each R⁴ is independently H, C₁₋₆alkyl, C₁₋₆haloalkyl, or C₃₋₁₀cycloalkyl.

Embodiment 33. The compound of any one of embodiments 1-32, or a stereoisomer or tautomer thereof, or a pharmaceutically acceptable salt of any of the foregoing, wherein R⁸ is —C(O)N(R⁴)₂, and each R⁴ is independently H, or C₁₋₆alkyl.

Embodiment 34. The compound of any one of embodiments 1-30, or a stereoisomer or tautomer thereof, or a pharmaceutically acceptable salt of any of the foregoing, wherein R⁸ is C₃₋₆cycloalkyl optionally substituted with one or more R^{a}.

Embodiment 35. The compound of any one of embodiments 1-30, or 34, or a stereoisomer or tautomer thereof, or a pharmaceutically acceptable salt of any of the foregoing, wherein R⁸ is selected from the group consisting of

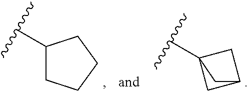 [SMILES: *C1CCCC1 ; *C12CC(C1)C2]

Embodiment 36. The compound of any one of embodiments 1-30, or a stereoisomer or tautomer thereof, or a pharmaceutically acceptable salt of any of the foregoing, wherein R⁸ is C₆₋₁₂aryl, optionally substituted with one or more R^{a}.

Embodiment 37. The compound of any one of embodiments 1-30, or 36, or a stereoisomer or tautomer thereof, or a pharmaceutically acceptable salt of any of the foregoing, wherein R⁸ is C₆₋₁₂aryl, optionally substituted with one or more —OH.

Embodiment 38. The compound of any one of embodiments 1-30, 36, or 37, or a stereoisomer or tautomer thereof, or a pharmaceutically acceptable salt of any of the foregoing, wherein R⁸ is selected from the group consisting of

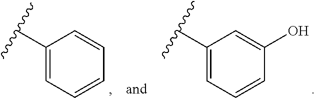 [SMILES: *c1ccccc1 ; *c1cccc(O)c1]

Embodiment 39. The compound of any one of embodiments 1-30, or a stereoisomer or tautomer thereof, or a pharmaceutically acceptable salt of any of the foregoing, wherein R⁸ is 3-6 membered heterocyclyl, optionally substituted with one or more R^{a}.

Embodiment 40. The compound of any one of embodiments 1-30, or 39, or a stereoisomer or tautomer thereof, or a pharmaceutically acceptable salt of any of the foregoing, wherein R⁸ is

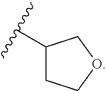 [SMILES: *C1CCOC1]

Embodiment 41. The compound of any one of embodiments 1-30, or a stereoisomer or tautomer thereof, or a pharmaceutically acceptable salt of any of the foregoing, wherein R⁸ is 5-10 membered heteroaryl, optionally substituted with one or more R^{a}.

Embodiment 42. The compound of any one of embodiments 1-30, or 41, or a stereoisomer or tautomer thereof, or a pharmaceutically acceptable salt of any of the foregoing, wherein R⁸ is 5-10 membered heteroaryl, optionally substituted with one or more halo, C₁₋₆alkyl, C₁₋₆haloalkyl, or C₃₋₁₀cycloalkyl.

Embodiment 43. The compound of any one of embodiments 1-30, 41, or 42, or a stereoisomer or tautomer thereof, or a pharmaceutically acceptable salt of any of the foregoing, wherein R is selected from the group consisting of

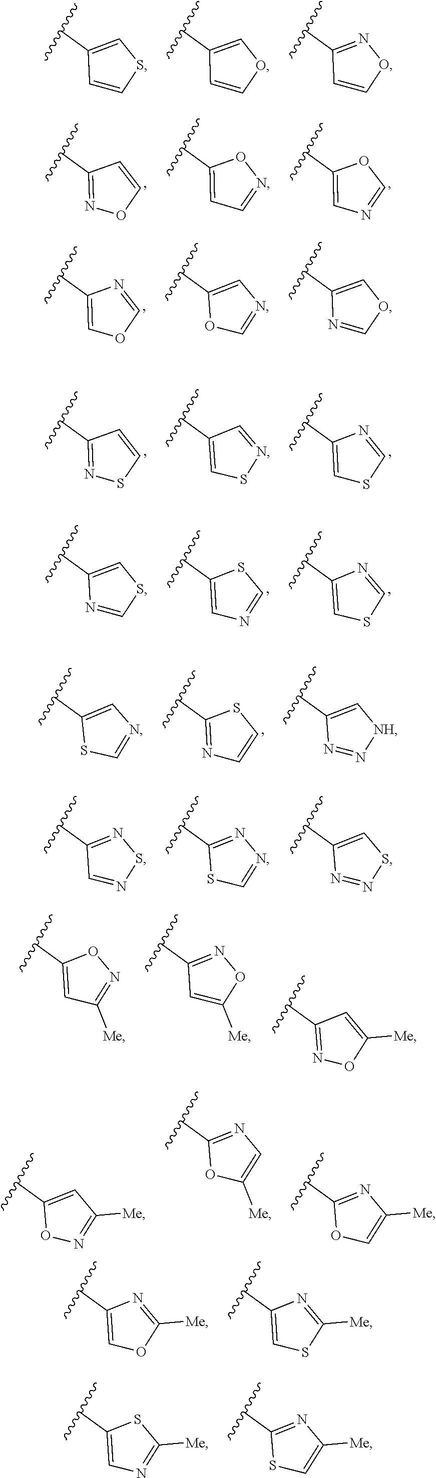

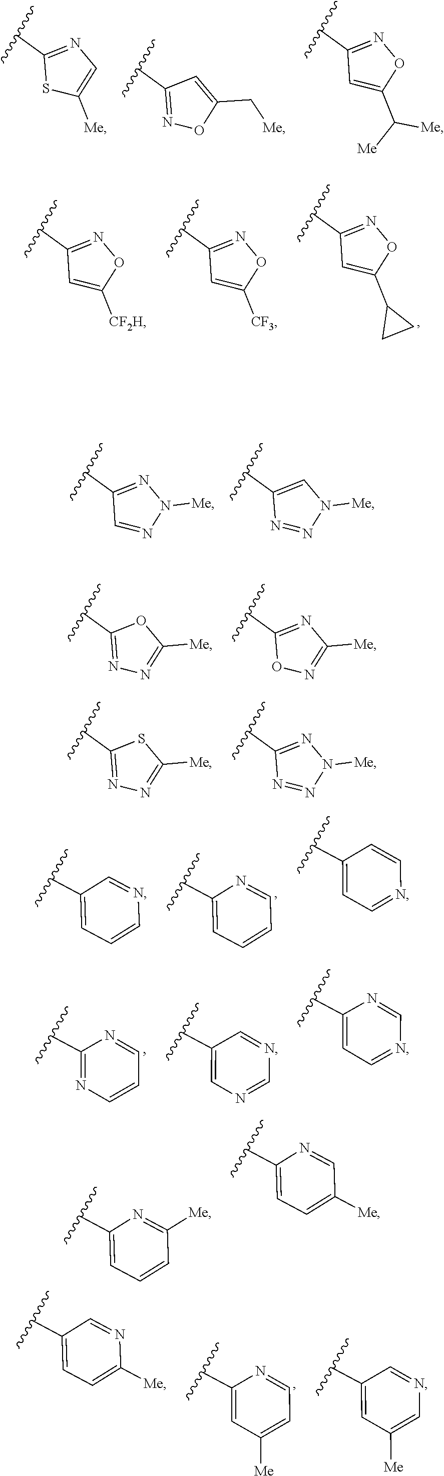

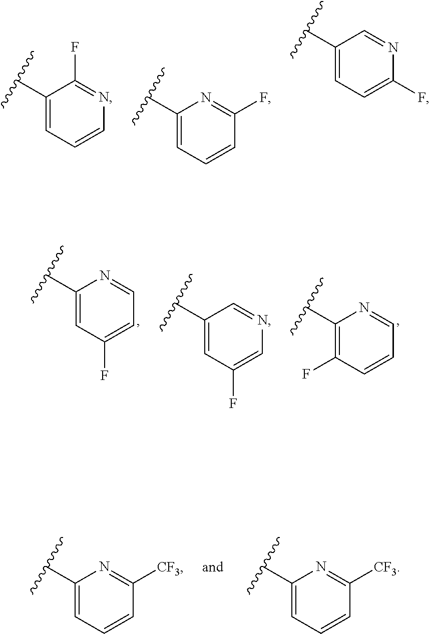

Embodiment 44. The compound of any one of embodiments 1-43, or a stereoisomer or tautomer thereof, or a pharmaceutically acceptable salt of any of the foregoing, wherein R^{11a} and R^{11b} are each, independently at each occurrence, H.

Embodiment 45. The compound of any one of embodiments 1-43, or a stereoisomer or tautomer thereof, or a pharmaceutically acceptable salt of any of the foregoing, wherein one of R^{11a} and R^{11b} is H, and the other of R^{11a} and R^{11b} is C₁₋₃alkyl.

Embodiment 46. The compound of any one of embodiments 1-43, or 45, or a stereoisomer or tautomer thereof, or a pharmaceutically acceptable salt of any of the foregoing, wherein one of R^{11a} and R^{11b} is H, and the other of R^{11a} and R^{11b} is methyl.

Embodiment 47. The compound of any one of embodiments 1-46, or a stereoisomer or tautomer thereof, or a pharmaceutically acceptable salt of any of the foregoing, wherein X is —O—.

Embodiment 48. The compound of any one of embodiments 1-46, or a stereoisomer or tautomer thereof, or a pharmaceutically acceptable salt of any of the foregoing, wherein X is —S—.

Embodiment 49. The compound of any one of embodiments 1-46, or a stereoisomer or tautomer thereof, or a pharmaceutically acceptable salt of any of the foregoing, wherein X is —C(R^{12a})(R^{12b})—.

Embodiment 50. The compound of any one of embodiments 1-46, or 49, or a stereoisomer or tautomer thereof, or a pharmaceutically acceptable salt of any of the foregoing, wherein X is —CH₂—, or —CH(C₁₋₆alkyl)-.

Embodiment 51. The compound of any one of embodiments 1-46, or a stereoisomer or tautomer thereof, or a pharmaceutically acceptable salt of any of the foregoing, wherein X is —N(R¹²)—.

Embodiment 52. The compound of any one of embodiments 1-46, or 51, or a stereoisomer or tautomer thereof, or a pharmaceutically acceptable salt of any of the foregoing, wherein X is —NH—.

Embodiment 53. The compound of any one of embodiments 1-46, wherein, n is 1, one of R^{12a} and R^{12b} is H, or C₁₋₆alkyl, and the other of R^{12a} and R^{12b} is taken together with R^{11a} or R^{11b} to form a C₃₋₁₀cycloalkyl.

Embodiment 54. The compound of any one of embodiments 1-10, 13-15, or 22-53, or a stereoisomer or tautomer thereof, or a pharmaceutically acceptable salt of any of the foregoing, wherein the compound is of formula (I-C):

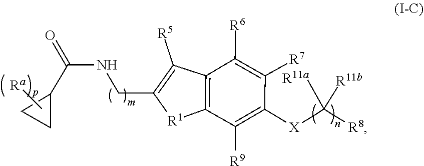

or a stereoisomer or tautomer thereof, or a pharmaceutically acceptable salt of any of the foregoing, wherein
p is an integer from 0-5.

Embodiment 55. The compound of any one of embodiments 1-10, 13-15, or 22-53, or a stereoisomer or tautomer thereof, or a pharmaceutically acceptable salt of any of the foregoing, wherein the compound is of formula (I-D):

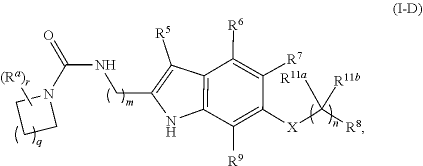

or a stereoisomer or tautomer thereof, or a pharmaceutically acceptable salt of any of the foregoing, wherein
q is an integer from 0-7; and
r is an integer from 0-19.

Embodiment 56. The compound of embodiment 1, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from Table 1.

Embodiment 57. A process for preparing a compound of formula (I), as defined in any one of embodiments 1-56, or a stereoisomer or tautomer thereof, or a pharmaceutically acceptable salt of any of the foregoing, wherein the process comprises:
(a) reacting a compound of formula (I-1):

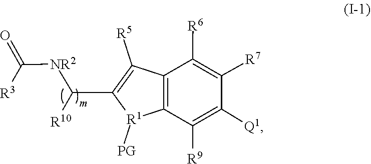

or a stereoisomer or tautomer thereof, or a pharmaceutically acceptable salt of any of the foregoing, wherein:
Q¹ is halo, boronic acid, or boronic ester;
PG is absent, or a protecting group;
m is an integer from 1 to 4;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R¹⁰ is H; and
each R^{a} is, independently at each occurrence, —OH, halo, C₁₋₆alkyl, C₁₋₆haloalkyl, or C₃₋₁₀cycloalkyl;
with a compound of formula (I-2):

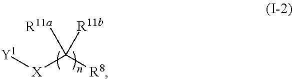

or a stereoisomer or tautomer thereof, or a pharmaceutically acceptable salt of any of the foregoing, wherein:
Y¹ is H, hydrazone, boronic acid, boronic ester, or halo;
n is an integer from 0 to 4;
X is —O—, —S—, —C(R^{12a})(R^{12b})—, —N(H)—, or —N(C₁₋₆alkyl)-;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl;
in the presence of one or more coupling reagent, to provide a compound of formula (I-3):

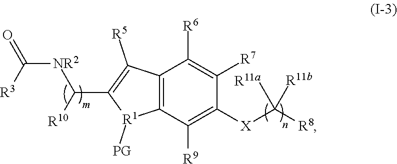

or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is, independently at each occurrence, —OH, halo, C₁₋₆alkyl, C₁₋₆haloalkyl, or C₃₋₁₀cycloalkyl;
followed by,
(b) optionally contacting the compound of formula (I-3) with a deprotecting agent;
to provide a compound of formula (I).

Embodiment 58. The process of embodiment 57, wherein the protecting group is a tert-butoxy group.

Embodiment 59. The process of embodiment 57, or embodiment 58, wherein the one or more coupling reagents comprises one or more reagents selected from the group consisting of a palladium catalyst, a phosphine ligand, and a base.

Embodiment 60. The process of any one of embodiments 57-59, wherein the deprotecting agent comprises an acid.

Embodiment 61. A process for preparing a compound of formula (I), as defined in any one of embodiments 1-56, or a stereoisomer or tautomer thereof, or a pharmaceutically acceptable salt of any of the foregoing, wherein the process comprises:
reacting a compound of formula (II-1):

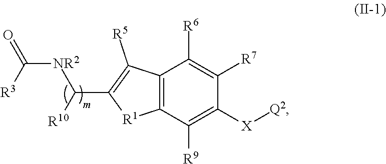

or a stereoisomer or tautomer thereof, or a pharmaceutically acceptable salt of any of the foregoing, wherein:
Q² is H, or (═O);
m is an integer from 1 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R¹⁰ is H;
R^{12a} and R^{12b} are each independently, H, or C₁₋₆alkyl; and
each R^{a} is, independently at each occurrence, —OH, halo, C₁₋₆alkyl, C₁₋₆haloalkyl, or C₃₋₁₀cycloalkyl;
with a compound of formula (II-2):

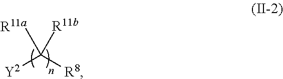

or a stereoisomer or tautomer thereof, or a pharmaceutically acceptable salt of any of the foregoing, wherein:
Y² is OH, (═O), halo, triphenylphosphonium salt, or sulfonic ester;
n is an integer from 0 to 4;
X is —O—, —S—, —C(R^{12a})(R^{12b})—, —N(H)—, or —N(C₁₋₆alkyl)-;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl; and
each R^{a} is, independently at each occurrence, —OH, halo, C₁₋₆alkyl, C₁₋₆haloalkyl, or C₃₋₁₀cycloalkyl;
in the presence of one or more coupling reagent, to provide a compound of formula (I).

Embodiment 62. The process of embodiments 60, wherein the compound of formula (II-1) is an amine, the compound of formula (II-2) is an aldehyde or ketone, and the reaction is a reductive amination.

Embodiment 63. The process of embodiments 60, wherein the compound of formula (II-1) is a phenol, or thiol, the compound of formula (II-2) is an alkyl halide or sulfonic ester and the reaction is an alkylation reaction.

Embodiment 64. A process for preparing a compound of formula (I), as defined in any one of embodiments 1-56, or a stereoisomer or tautomer thereof, or a pharmaceutically acceptable salt of any of the foregoing, comprises:
reacting a compound of formula (III-1):

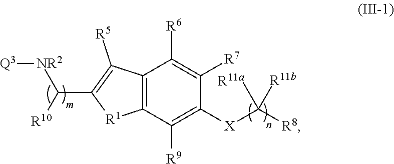

or a stereoisomer or tautomer thereof, or a pharmaceutically acceptable salt of any of the foregoing, wherein:
Q³ is H;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl;
with a compound of formula (III-2):

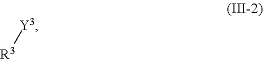

or a stereoisomer or tautomer thereof, or a pharmaceutically acceptable salt of any of the foregoing, wherein:
Y³ is —C(O)OH, or —C(O)-halo;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a}; and
each R^{a} is, independently at each occurrence, —OH, halo, C₁₋₆alkyl, C₁₋₆haloalkyl, or C₃₋₁₀cycloalkyl;
in the presence of one or more coupling reagent, to provide a compound of formula (I).

Embodiment 65. The process of embodiment 64, wherein the one or more coupling reagents comprises one or more reagents selected from the group consisting of HATU and DIEA.

Embodiment 66. A pharmaceutical composition comprising (i) a compound of any one of embodiments 1-56, or a stereoisomer or tautomer thereof, or a pharmaceutically acceptable salt of any of the foregoing, and (ii) one or more pharmaceutically acceptable excipients.

Embodiment 67. A method of modulating SLC6A19 in a cell, comprising exposing the cell to a composition comprising an effective amount of a compound of any one or embodiments 1-56, or a stereoisomer or tautomer thereof, or a pharmaceutically acceptable salt of any of the foregoing, or a pharmaceutical composition of embodiment 66.

Embodiment 68. A method of inhibiting SLC6A19 in a cell, comprising exposing the cell to a composition comprising an effective amount of a compound of any one or embodiments 1-56, or a stereoisomer or tautomer thereof, or a pharmaceutically acceptable salt of any of the foregoing, or a pharmaceutical composition of embodiment 66.

Embodiment 69. A method of reducing systemic phenylalanine, tyrosine, glutamine, or glycine levels in an individual in need thereof, comprising administering to the individual an effective amount of a compound of any one of embodiments 1-56, or a stereoisomer or tautomer thereof, or a pharmaceutically acceptable salt of any of the foregoing, or a pharmaceutical composition of embodiment 66.

Embodiment 70. A method of treating a SLC6A19-mediated disease, disorder, or condition in an individual in need thereof, comprising administering to the individual an effective amount of a compound of any one of embodiments 1-56, or a stereoisomer or tautomer thereof, or a pharmaceutically acceptable salt of any of the foregoing, or a pharmaceutical composition of embodiment 66.

Embodiment 71. The method of embodiment 70, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

Embodiment 72. The method of embodiment 70 or embodiment 71, wherein the disease, disorder, or condition is selected from the group consisting of phenylketonuria (PKU), chronic kidney disease (CKD), metabolic syndrome, and metabolic diseases.

Embodiment 73. The method of embodiment 70, wherein the disease, disorder, or condition is associated with abnormal levels of amino acids.

Embodiment 74. The method of embodiment 70, or embodiment 74, wherein the disease, disorder, or condition is associated with a genetic defect in phenylalanine hydroxylase.

Embodiment 75. A kit, comprising (i) a compound of any one of embodiments 1-56, or a stereoisomer or tautomer thereof, or a pharmaceutically acceptable salt of any of the foregoing, or a pharmaceutical composition of embodiment 66, and (ii) instructions for use in treating an SLC6A19-mediated disease, disorder, or condition in an individual in need thereof.

Embodiment 76. The kit of embodiment 75, wherein the disease, disorder, or condition is associated with abnormal levels of amino acids.

Embodiment 77. The kit of embodiment 75, or embodiment 76, wherein the disease, disorder, or condition is associated with a genetic defect in phenylalanine hydroxylase.

Embodiment 78. The kit of embodiment 75, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

Embodiment 79. The kit of embodiment 75, wherein the individual has a genetic defect in phenylalanine hydroxylase.

Embodiment 14B. The compound of any one of embodiments 1-10, or 13, or a stereoisomer or tautomer thereof, or a pharmaceutically acceptable salt of any of the foregoing, wherein R³ is C₃₋₆cycloalkyl optionally substituted with one or more halo, C₁₋₃alkyl, or C₁₋₃haloalkyl.

Embodiment 15B. The compound of any one of embodiments 1-10, 13, or 14 or a stereoisomer or tautomer thereof, or a pharmaceutically acceptable salt of any of the foregoing, wherein R³ is selected from the group consisting of

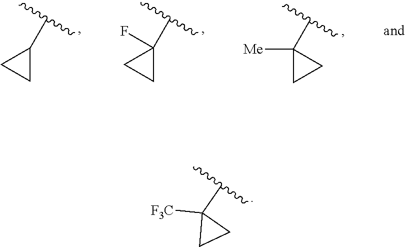

Embodiment 56B. The compound of any one of embodiments 1-29, 43-55, or 14B-15B, or a stereoisomer or tautomer thereof, or a pharmaceutically acceptable salt of any of the foregoing, wherein the compound is of formula (I-H):

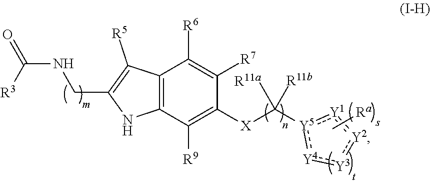

or a stereoisomer or tautomer thereof, or a pharmaceutically acceptable salt of any of the foregoing, wherein
s is an integer from 0-6;
t is 1 or 2; and
[symbol] represents a single or double bond.

Embodiment 57B. The compound of embodiment 56B, or a stereoisomer or tautomer thereof, or a pharmaceutically acceptable salt of any of the foregoing, wherein the compound is of formula:

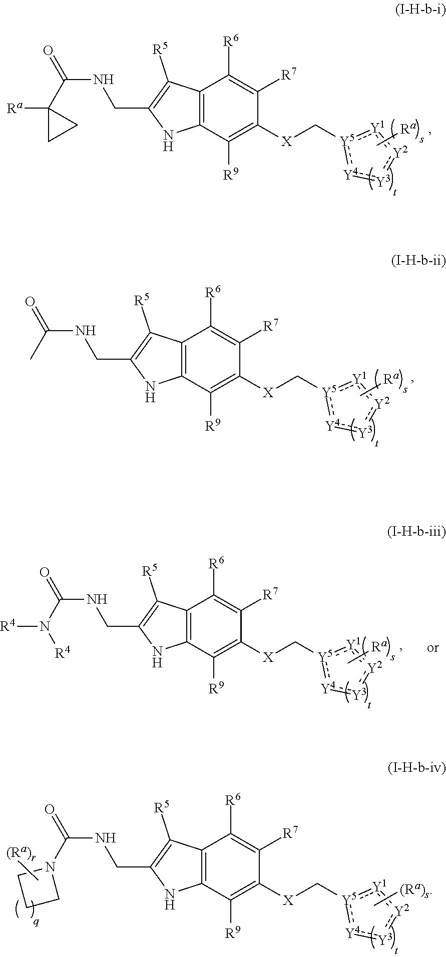

Embodiment 63B. A process for preparing a compound of formula (I), as defined in any one of embodiments 1-58, 14B-15B, or 57B-58B, or a stereoisomer or tautomer thereof, or a pharmaceutically acceptable salt of any of the foregoing, comprises:
(a) reacting a compound of formula (IV-1):

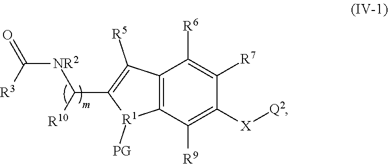

or a stereoisomer or tautomer thereof, or a pharmaceutically acceptable salt of any of the foregoing, wherein:
PG is absent or a protecting group;
Q² is H, or (═O);
m is an integer from 1 to 4;
X is —O—, —S—, —C(R^{12a})(R^{12b})—, —N(H)—, or —N(C₁₋₆alkyl)-;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
the C₁₋₆alkyl of R⁵ and R⁷ are each independently optionally substituted with one or more halo, or CN;
R⁶ and R⁹ are each independently H, or halo;
R¹⁰ is H;
R^{12a} and R^{12b} are each independently, H, or C₁₋₆alkyl; and
each R^{a} is, independently at each occurrence, —OH, halo, C₁₋₆alkyl, C₁₋₆haloalkyl, or C₃₋₁₀cycloalkyl;
with a compound of formula (II-2):

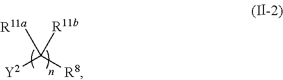

or a stereoisomer or tautomer thereof, or a pharmaceutically acceptable salt of any of the foregoing, wherein:
Y² is OH, (═O), halo, triphenylphosphonium salt, or sulfonic ester;
n is an integer from 0 to 4;
X is —O—, —S—, —C(R^{12a})(R^{12b})—, —N(H)—, or —N(C₁₋₆alkyl)-;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl; and
each R^{a} is, independently at each occurrence, —OH, halo, C₁₋₆alkyl, C₁₋₆haloalkyl, or C₃₋₁₀cycloalkyl;
in the presence of one or more coupling reagents, to provide a compound of formula (I-3):

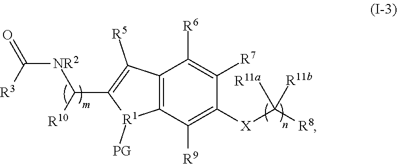

or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
the C₁₋₆alkyl of R⁵ and R⁷ are each independently optionally substituted with one or more halo, or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is, independently at each occurrence, —OH, halo, C₁₋₆alkyl, C₁₋₆haloalkyl, or C₃₋₁₀cycloalkyl;
followed by,
(b) optionally contacting the compound of formula (I-3) with a deprotecting agent;
to provide a compound of formula (I).

Embodiment 64B. The process of embodiment 63B, wherein the protecting group is absent.

Embodiment 65B. The process of embodiment 63B, wherein the protecting group is a mesylate or tosylate.

Embodiment 66B. The process of any one of embodiments 63B-65B, wherein the compound of formula (IV-1) is an amine, the compound of formula (II-2) is an aldehyde or ketone, and the reaction is a reductive amination.

Embodiment 67B. The process of any one of embodiments 63B-65B, wherein the compound of formula (IV-1) is a phenol, or thiol, the compound of formula (II-2) is an alkyl halide or sulfonic ester and the reaction is an alkylation reaction.

Embodiment 68B. The process of embodiment 63B or 65B-67B, wherein the deprotecting agent comprises Mg.

Embodiment 1C. A compound of formula (I′):

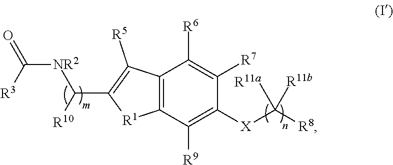

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆ alkoxy, C₃₋₁₀cycloalkyl, or C₁₋₆haloalkyl, wherein
   the C₁₋₆ alkyl of R⁵ and R⁷ are each independently optionally substituted with one or more halo or CN;
R⁶ and R⁹ are each independently H or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy.

Embodiment 2C. The compound of embodiment 1C, wherein the compound is a compound of formula (I):

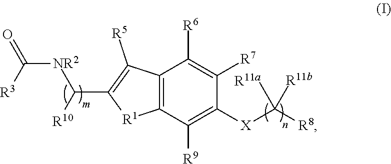

or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₃₋₁₀cycloalkyl or C₁₋₆haloalkyl, wherein
   the C₁₋₆alkyl of R⁵ and R⁷ are each independently optionally substituted with one or more halo, or CN;
R⁶ and R⁹ are each independently H, or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is, independently at each occurrence, —OH, halo, C₁₋₆alkyl, C₁₋₆haloalkyl, or C₃₋₁₀cycloalkyl.

Embodiment 3C. The compound of embodiment 1C, or a stereoisomer or tautomer thereof, or a pharmaceutically acceptable salt of any of the foregoing, wherein the compound is a compound of formula (I-A):

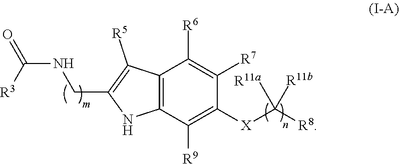

Embodiment 4C. The compound of embodiment 1C, or embodiment 2C, or a stereoisomer or tautomer thereof, or a pharmaceutically acceptable salt of any of the foregoing, wherein m is an integer from 1-2.

Embodiment 5C. The compound of any of embodiments 1C-4C, or a stereoisomer or tautomer thereof, or a pharmaceutically acceptable salt of any of the foregoing, wherein m is 1.

Embodiment 6C. The compound of any of embodiments 1C-5C, or a stereoisomer or tautomer thereof, or a pharmaceutically acceptable salt of any of the foregoing, wherein n is an integer from 0-2.

Embodiment 7C. The compound of any of embodiments 1C-6C, or a stereoisomer or tautomer thereof, or a pharmaceutically acceptable salt of any of the foregoing, wherein n is 1.

Embodiment 8C. The compound of any of embodiments 1C-6C, or a stereoisomer or tautomer thereof, or a pharmaceutically acceptable salt of any of the foregoing, wherein n is 0.

Embodiment 9C. The compound of any of embodiments 1C-7C, or a stereoisomer or tautomer thereof, or a pharmaceutically acceptable salt of any of the foregoing, wherein m is 1 and n is 1.

Embodiment 10C. The compound of any of embodiments 1C-6C, or a stereoisomer or tautomer thereof, or a pharmaceutically acceptable salt of any of the foregoing, wherein m is 1 and n is 1.

Embodiment 11C. The compound of any of embodiments 1C-10C, or a stereoisomer or tautomer thereof, or a pharmaceutically acceptable salt of any of the foregoing, wherein R³ is C₁₋₃alkyl, C₁₋₃haloalkyl, C₃₋₆cycloalkyl, —N(R⁴)₂, or 4-6 membered heterocyclyl, wherein each R³ is optionally substituted with one or more R^{a}.

Embodiment 12C. The compound of any of embodiments 1C-11C, or a stereoisomer or tautomer thereof, or a pharmaceutically acceptable salt of any of the foregoing, wherein R³ is C₁₋₃alkyl.

Embodiment 13C. The compound of any of embodiments 1C-12C, or a stereoisomer or tautomer thereof, or a pharmaceutically acceptable salt of any of the foregoing, wherein R³ is methyl, ethyl, or isopropyl.

Embodiment 14C. The compound of any of embodiments 1C-11C, or a stereoisomer or tautomer thereof, or a pharmaceutically acceptable salt of any of the foregoing, wherein R³ is C₁₋₃alkyl, optionally substituted with one or more R^{a}; a is —OH, halo, C₁₋₃alkoxy, 4-6 membered heterocyclyl, or C₃₋₆cycloalkyl, wherein the C₁₋₃alkoxy, 4-6 membered heterocyclyl or C₃₋₆cycloalkyl of R^{a} is optionally substituted with one or more R^{b}; and each R^{b} is, independently at each occurrence, —OH, halo, C₁₋₃alkyl or C₁₋₃alkoxy.

Embodiment 15C. The compound of any of embodiments 1C-11C, or 14C, or a stereoisomer or tautomer thereof, or a pharmaceutically acceptable salt of any of the foregoing, wherein R³ is selected from the group consisting of

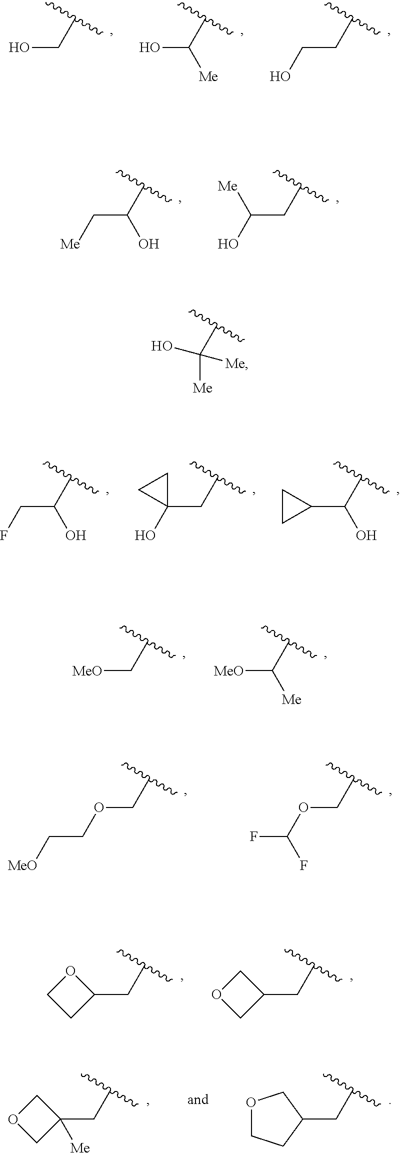

Embodiment 16C. The compound of any of embodiments 1C-11C, or a stereoisomer or tautomer thereof, or a pharmaceutically acceptable salt of any of the foregoing, wherein R³ is C₃₋₆cycloalkyl optionally substituted with one or more R^{a}.

Embodiment 17C. The compound of any of embodiments 1C-11C, or 16C, or a stereoisomer or tautomer thereof, or a pharmaceutically acceptable salt of any of the foregoing, wherein R³ is C₃₋₆cycloalkyl optionally substituted with one or more —OH, halo, C₁₋₃alkyl, C₁₋₃haloalkyl, wherein the C₁₋₃alkyl is optionally substituted with one or more —OH, or C₁₋₃alkoxy.

Embodiment 18C. The compound of any of embodiments 1C-11C, 16C, or 17C or a stereoisomer or tautomer thereof, or a pharmaceutically acceptable salt of any of the foregoing, wherein R³ is selected from the group consisting of

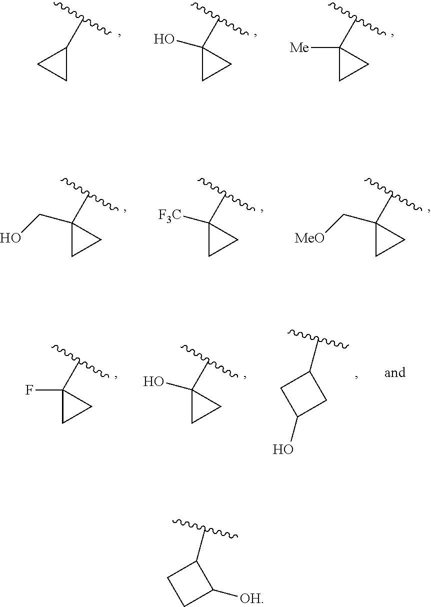

Embodiment 19C. The compound of any of embodiments 1C-11C, or a stereoisomer or tautomer thereof, or a pharmaceutically acceptable salt of any of the foregoing, wherein R³ is —N(R⁴)₂, and each R⁴ is independently H, C₁₋₃alkyl, C₁₋₃haloalkyl, or C₃₋₆cycloalkyl.

Embodiment 20C. The compound of any of embodiments 1C-11C, or 19C, or a stereoisomer or tautomer thereof, or a pharmaceutically acceptable salt of any of the foregoing, wherein R³ is —N(R⁴)₂, and each R⁴ is independently H, or C₁₋₃alkyl.

Embodiment 21C. The compound of any of embodiments 1C-11C, or a stereoisomer or tautomer thereof, or a pharmaceutically acceptable salt of any of the foregoing, wherein R³ is 4-6 membered heterocyclyl, optionally substituted with one or more R^{a}.

Embodiment 22C. The compound of any of embodiments 1C-11C, or 21C, or a stereoisomer or tautomer thereof, or a pharmaceutically acceptable salt of any of the foregoing, wherein R³ is —N(R⁴)₂, and both R⁴ are taken together with the N atom to which they are attached to form a 4-6 membered heterocyclyl optionally substituted with one or more R^{a}.

Embodiment 23C. The compound of any of embodiments 1C-11C, 21C, or 22C, or a stereoisomer or tautomer thereof, or a pharmaceutically acceptable salt of any of the foregoing, wherein R³ is —N(R⁴)₂, and both R⁴ are taken together with the N atom to which they are attached to form a 4-6 membered heterocyclyl optionally substituted with one or more halo, C₁₋₃alkyl, or C₁₋₃haloalkyl.

Embodiment 24C. The compound of any of embodiments 1C-11C, or 21C-23C, or a stereoisomer or tautomer thereof, or a pharmaceutically acceptable salt of any of the foregoing, wherein R³ is selected from the group consisting of

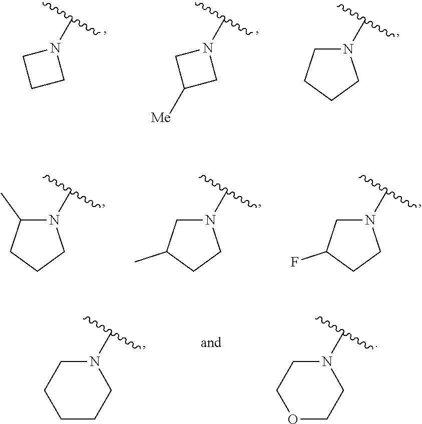

Embodiment 25C. The compound of any of embodiments 1C-11C, or 21C, or a stereoisomer or tautomer thereof, or a pharmaceutically acceptable salt of any of the foregoing, wherein R³ is 4-6 membered heterocyclyl optionally substituted with one or more —CN, halo, or C₁₋₃alkyl, wherein the C₁₋₃alkyl of R^{a} is optionally substituted with one or more OH.

Embodiment 26C. The compound of any of embodiments 1C-11C, 21C, or 25C, or a stereoisomer or tautomer thereof, or a pharmaceutically acceptable salt of any of the foregoing, wherein R³ is selected from the group consisting of

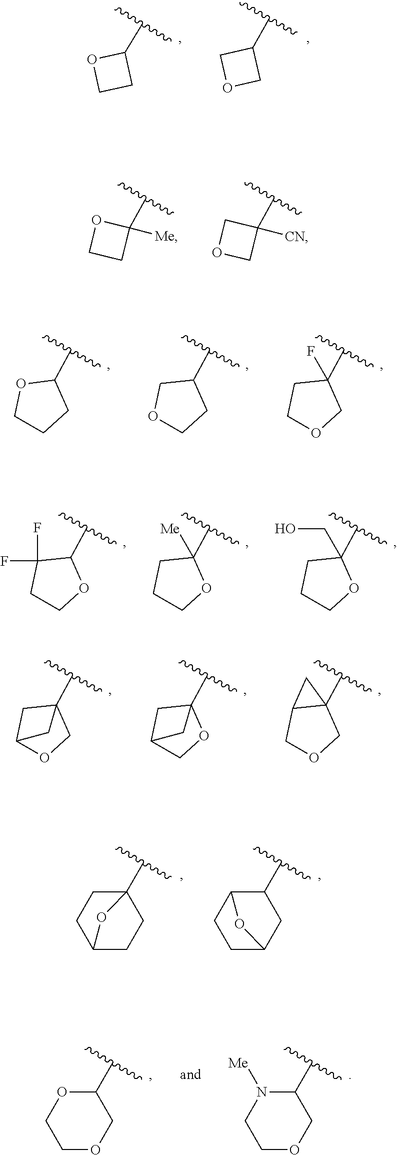

Embodiment 27C. The compound of any of embodiments 1C-26C, or a stereoisomer or tautomer thereof, or a pharmaceutically acceptable salt of any of the foregoing, wherein R⁵ and R⁷ are each independently H, halo, C₁₋₃alkyl, C₃₋₆cycloalkyl or C₁₋₃haloalkyl, wherein the C₁₋₃alkyl of R⁵ and R⁷ are each independently optionally substituted with one or more halo, or CN.

Embodiment 28C. The compound of any of embodiments 1C-27C, or a stereoisomer or tautomer thereof, or a pharmaceutically acceptable salt of any of the foregoing, wherein R⁵ and R⁷ are each independently H, halo, C₁₋₃alkyl, or C₁₋₃haloalkyl.

Embodiment 29C. The compound of any of embodiments 1C-28C, or a stereoisomer or tautomer thereof, or a pharmaceutically acceptable salt of any of the foregoing, wherein R⁵ and R⁷ are each independently H.

Embodiment 30C. The compound of any of embodiments 1C-28C, or a stereoisomer or tautomer thereof, or a pharmaceutically acceptable salt of any of the foregoing, wherein one of R⁵ and R⁷ is H, and the other of R⁵ and R⁷ is independently halo, C₁₋₃alkyl, or C₁₋₃haloalkyl.

Embodiment 31C. The compound of any of embodiments 1C-30C, or a stereoisomer or tautomer thereof, or a pharmaceutically acceptable salt of any of the foregoing, wherein R⁶ and R⁹ are each independently H, or halo.

Embodiment 32C. The compound of any of embodiments 1C-31C, or a stereoisomer or tautomer thereof, or a pharmaceutically acceptable salt of any of the foregoing, wherein R⁶ and R⁹ are each independently H, F, or Cl.

Embodiment 33C. The compound of any of embodiments 1C-32C, or a stereoisomer or tautomer thereof, or a pharmaceutically acceptable salt of any of the foregoing, wherein R⁶ and R⁹ are each independently H.

Embodiment 34C. The compound of any of embodiments 1C-32C, or a stereoisomer or tautomer thereof, or a pharmaceutically acceptable salt of any of the foregoing, wherein one of R⁶ and R⁹ is H, and the other of R⁶ and R⁹ is F, or Cl.

Embodiment 35C. The compound of any of embodiments 1C-34C, or a stereoisomer or tautomer thereof, or a pharmaceutically acceptable salt of any of the foregoing, wherein R⁸ is —C(O)N(R₄)₂, C₃₋₆cycloalkyl, C₆₋₁₂aryl, 3-6 membered heterocyclyl, or 5-10 membered heteroaryl, wherein each R⁸ is optionally substituted with one or more R^{a}.

Embodiment 36C. The compound of any of embodiments 1C-35C, or a stereoisomer or tautomer thereof, or a pharmaceutically acceptable salt of any of the foregoing, wherein R⁸ is —C(O)N(R₄)₂.

Embodiment 37C. The compound of any of embodiments 1C-36C, or a stereoisomer or tautomer thereof, or a pharmaceutically acceptable salt of any of the foregoing, wherein R⁸ is —C(O)N(R₄)₂, and each R⁴ is independently H, C₁₋₆alkyl, C₁₋₆haloalkyl, or C₃₋₁₀cycloalkyl.

Embodiment 38C. The compound of any of embodiments 1C-37C, or a stereoisomer or tautomer thereof, or a pharmaceutically acceptable salt of any of the foregoing, wherein R⁸ is —C(O)N(R₄)₂, and each R⁴ is independently H, or C₁₋₆alkyl.

Embodiment 39C. The compound of any of embodiments 1C-35C, or a stereoisomer or tautomer thereof, or a pharmaceutically acceptable salt of any of the foregoing, wherein R⁸ is C₃₋₆cycloalkyl optionally substituted with one or more R^{a}.

Embodiment 40C. The compound of any of embodiments 1C-35C, or 39C, or a stereoisomer or tautomer thereof, or a pharmaceutically acceptable salt of any of the foregoing, wherein R⁸ is selected from the group consisting of

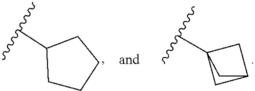 [SMILES: *C1CCCC1 ; *C12CC(C1)C2]

Embodiment 41C. The compound of any of embodiments 1C-35C, or a stereoisomer or tautomer thereof, or a pharmaceutically acceptable salt of any of the foregoing, wherein R⁸ is C₆₋₁₂aryl, optionally substituted with one or more R^{a}.

Embodiment 42C. The compound of any of embodiments 1C-35C, or 41C, or a stereoisomer or tautomer thereof, or a pharmaceutically acceptable salt of any of the foregoing, wherein R⁸ is C₆₋₁₂aryl, optionally substituted with one or more —OH.

Embodiment 43C. The compound of any of embodiments 1C-35C, 41C, or 42C, or a stereoisomer or tautomer thereof, or a pharmaceutically acceptable salt of any of the foregoing, wherein R⁸ is selected from the group consisting of

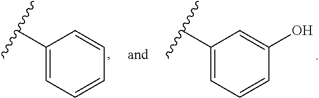 [SMILES: *c1ccccc1 ; *c1cccc(O)c1]

Embodiment 44C. The compound of any of embodiments 1C-35C, or a stereoisomer or tautomer thereof, or a pharmaceutically acceptable salt of any of the foregoing, wherein R⁸ is 3-6 membered heterocyclyl, optionally substituted with one or more R^{a}.

Embodiment 45C. The compound of any of embodiments 1C-35C, or 44C, or a stereoisomer or tautomer thereof, or a pharmaceutically acceptable salt of any of the foregoing, wherein R⁸ is

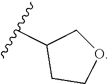 [SMILES: *C1CCOC1]

Embodiment 46C. The compound of any of embodiments 1C-35C, or a stereoisomer or tautomer thereof, or a pharmaceutically acceptable salt of any of the foregoing, wherein R⁸ is 5-10 membered heteroaryl, optionally substituted with one or more R^{a}.

Embodiment 47C. The compound of any of embodiments 1C-35C, or 46C, or a stereoisomer or tautomer thereof, or a pharmaceutically acceptable salt of any of the foregoing, wherein R⁸ is 5-10 membered heteroaryl, optionally substituted with one or more halo, C₁₋₆alkyl, C₁₋₆haloalkyl, or C₃₋₁₀cycloalkyl.

Embodiment 48C. The compound of any of embodiments 1C-35C, 46C, or 47C, or a stereoisomer or tautomer thereof, or a pharmaceutically acceptable salt of any of the foregoing, wherein R is selected from the group consisting of

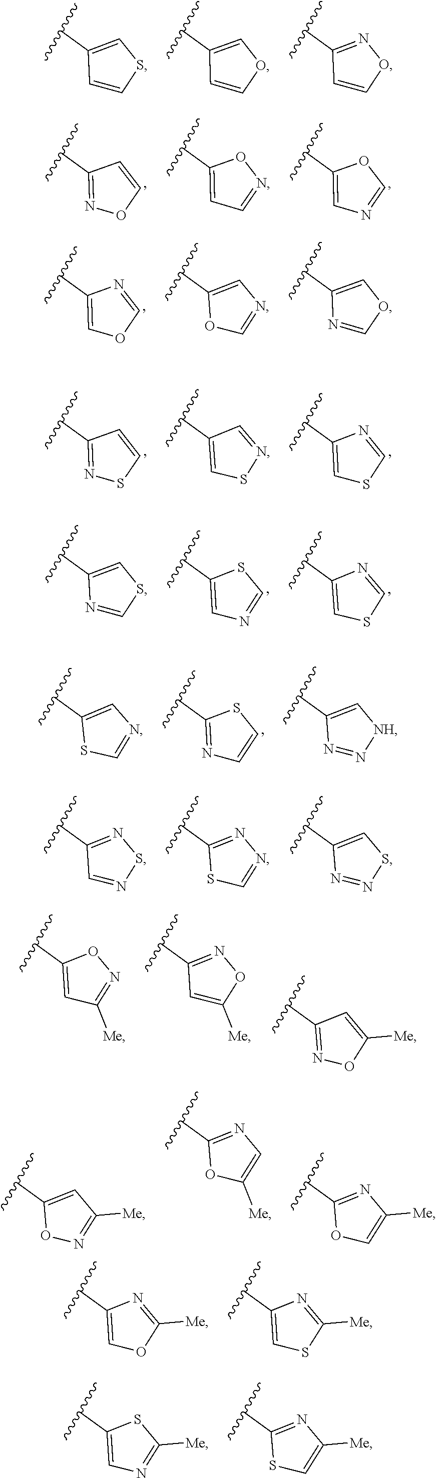

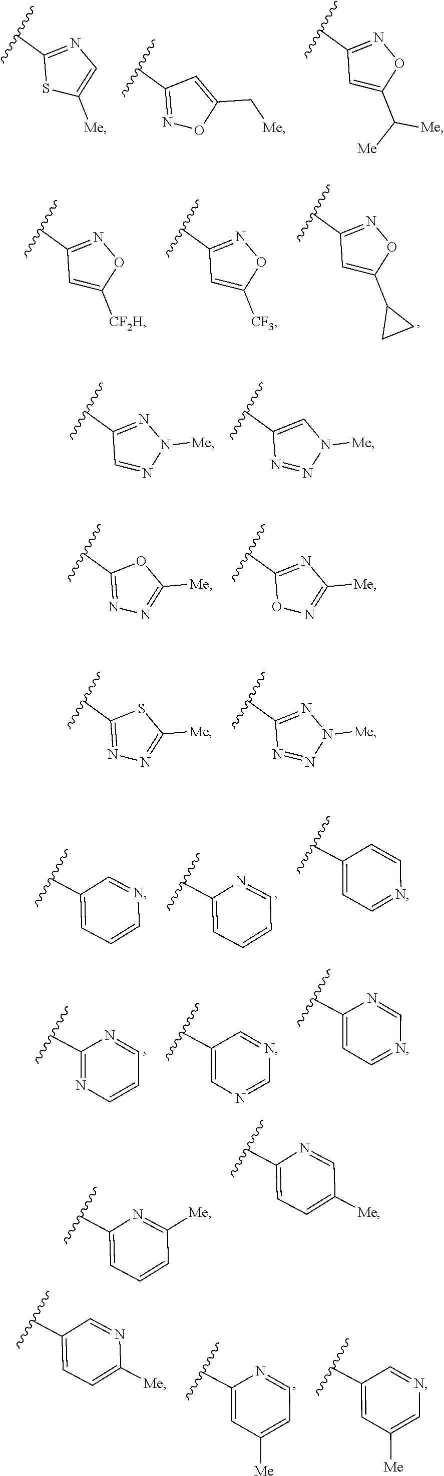

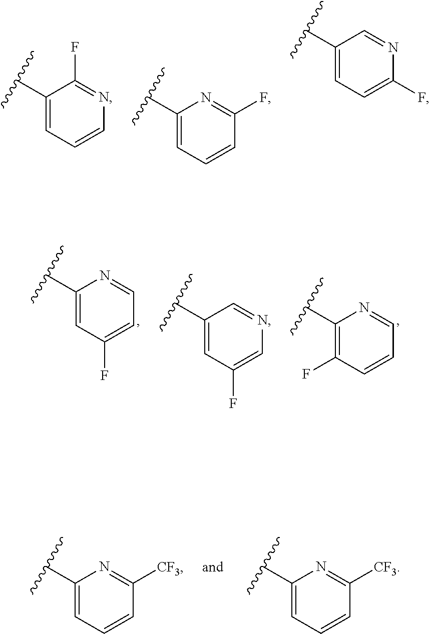

Embodiment 49C. The compound of any of embodiments 1C-48C, or a stereoisomer or tautomer thereof, or a pharmaceutically acceptable salt of any of the foregoing, wherein R^{11a} and R^{11b} are each, independently at each occurrence, H.

Embodiment 50C. The compound of any of embodiments 1C-48C, or a stereoisomer or tautomer thereof, or a pharmaceutically acceptable salt of any of the foregoing, wherein one of R^{11a} and R^{11b} is H, and the other of R^{11a} and R^{11b} is C₁₋₃alkyl.

Embodiment 51C. The compound of any of embodiments 1C-48C, or 50C, or a stereoisomer or tautomer thereof, or a pharmaceutically acceptable salt of any of the foregoing, wherein one of R^{11a} and R^{11b} is H, and the other of R^{11a} and R^{11b} is methyl.

Embodiment 52C. The compound of any of embodiments 1C-51C, or a stereoisomer or tautomer thereof, or a pharmaceutically acceptable salt of any of the foregoing, wherein X is —O—.

Embodiment 53C. The compound of any of embodiments 1C-51C, or a stereoisomer or tautomer thereof, or a pharmaceutically acceptable salt of any of the foregoing, wherein X is —S—.

Embodiment 54C. The compound of any of embodiments 1C-51C, or a stereoisomer or tautomer thereof, or a pharmaceutically acceptable salt of any of the foregoing, wherein X is —C(R^{12a})(R^{12b})—.

Embodiment 55C. The compound of any of embodiments 1C-51C, or 54C, or a stereoisomer or tautomer thereof, or a pharmaceutically acceptable salt of any of the foregoing, wherein X is —CH₂—, or —CH(C₁₋₆alkyl)-.

Embodiment 56C. The compound of any of embodiments 1C-51C, or a stereoisomer or tautomer thereof, or a pharmaceutically acceptable salt of any of the foregoing, wherein X is —N(R¹²)—.

Embodiment 57C. The compound of any of embodiments 1C-51C, or 56C, or a stereoisomer or tautomer thereof, or a pharmaceutically acceptable salt of any of the foregoing, wherein X is —NH—.

Embodiment 58C. The compound of any of embodiments 1C-51C, wherein, n is 1, one of R^{12a} and R^{12b} is H, or C₁₋₆alkyl, and the other of R^{12a} and R^{12b} is taken together with R^{11a} or R^{11b} to form a C₃₋₁₀cycloalkyl.

Embodiment 59C. The compound of any of embodiments 1C-11C, 16C-18C, or 27C-58C, or a stereoisomer or tautomer thereof, or a pharmaceutically acceptable salt of any of the foregoing, wherein the compound is of formula (I-C):

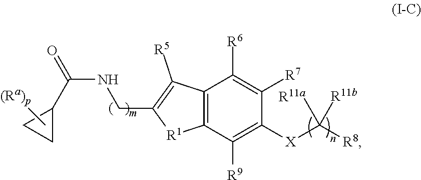

or a stereoisomer or tautomer thereof, or a pharmaceutically acceptable salt of any of the foregoing, wherein
p is an integer from 0-5.

Embodiment 60C. The compound of any of embodiments 1C-11C, 16C-18C, or 27C-58C, or a stereoisomer or tautomer thereof, or a pharmaceutically acceptable salt of any of the foregoing, wherein the compound is of formula (I-D):

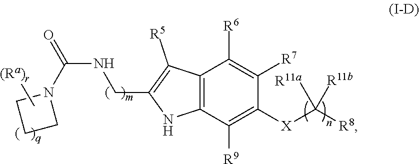

or a stereoisomer or tautomer thereof, or a pharmaceutically acceptable salt of any of the foregoing, wherein
q is an integer from 0-7; and
r is an integer from 0-19.

Embodiment 61C. The compound of any of embodiments 1C-34C, or 48C-60C, or a stereoisomer or tautomer thereof, or a pharmaceutically acceptable salt of any of the foregoing, wherein the compound is of formula (I-H):

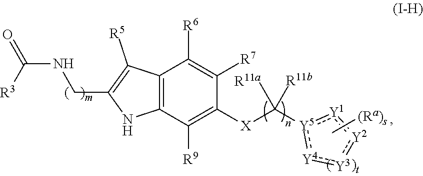

or a stereoisomer or tautomer thereof, or a pharmaceutically acceptable salt of any of the foregoing, wherein
s is an integer from 0-6;
t is 1 or 2; and
[symbol] represents a single or double bond.

Embodiment 62C. The compound of embodiment 61C, or a stereoisomer or tautomer thereof, or a pharmaceutically acceptable salt of any of the foregoing, wherein the compound is of formula:

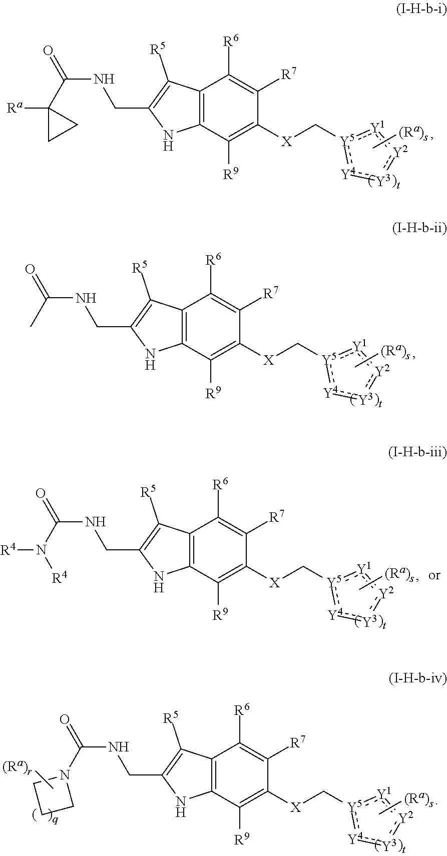

Embodiment 63C. The compound of embodiment 1C, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from Compounds 1-317 of Table 1.

Embodiment 64C. A process for preparing a compound of formula (I′), as defined in any of embodiments 1C-63C, or a stereoisomer or tautomer thereof, or a pharmaceutically acceptable salt of any of the foregoing, wherein the process comprises:
(a) reacting a compound of formula (I′-1):

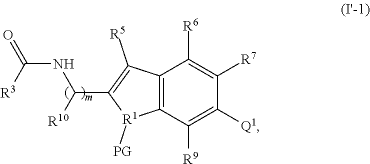

or a stereoisomer or tautomer thereof, or a pharmaceutically acceptable salt of any of the foregoing, wherein:
Q¹ is halo, boronic acid, or boronic ester;
PG is absent, or a protecting group;
m is an integer from 1 to 4;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
the C₁₋₆ alkyl of R⁵ and R⁷ are each independently optionally substituted with one or more halo or CN;
R⁶ and R⁹ are each independently H, or halo;
R¹⁰ is H; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (I′-2):

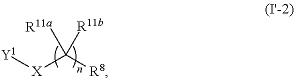

or a stereoisomer or tautomer thereof, or a pharmaceutically acceptable salt of any of the foregoing, wherein:
Y¹ is H, hydrazone, boronic acid, boronic ester, or halo;
n is an integer from 0 to 4;
X is —O—, —S—, —C(R^{12a})(R^{12b})—, —N(H)—, or —N(C₁₋₆ alkyl)-;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
in the presence of one or more coupling reagent, to provide a compound of formula (I′-3):

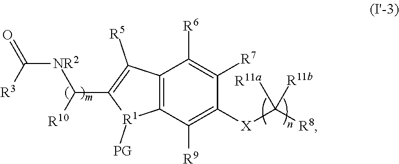

or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
   the C₁₋₆alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
followed by,
(b) optionally contacting the compound of formula (I′-3) with a deprotecting agent;
to provide a compound of formula (I′).

Embodiment 65C. The process of embodiment 64C, wherein the protecting group is a tert-butoxy group.

Embodiment 66C. The process of embodiment 64C, or 65C, wherein the one or more coupling reagents comprises one or more reagents selected from the group consisting of a palladium catalyst, a phosphine ligand, and a base.

Embodiment 67C. The process of any of embodiments 64C-66C, wherein the deprotecting agent comprises an acid.

Embodiment 68C. A process for preparing a compound of formula (I′), as defined in any of embodiments 1C-63C or a stereoisomer or tautomer thereof, or a pharmaceutically acceptable salt of any of the foregoing, comprises:
(a) reacting a compound of formula (IV′-1):

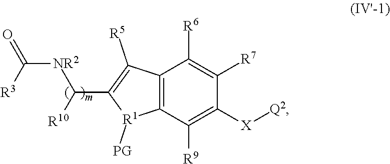

or a stereoisomer or tautomer thereof, or a pharmaceutically acceptable salt of any of the foregoing, wherein:
PG is absent or a protecting group;
Q² is H, or (═O);
m is an integer from 1 to 4;
X is —O—, —S—, —C(R^{12a})(R^{12b})—, —N(H)—, or —N(C₁₋₆ alkyl)-;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
   the C₁₋₆ alkyl of R⁵ and R⁷ are each independently optionally substituted with one or more halo or CN;
R⁶ and R⁹ are each independently H, or halo;
R¹⁰ is H;
R^{12a} and R^{12b} are each independently, H, or C₁₋₆alkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (II′-2):

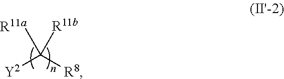

or a stereoisomer or tautomer thereof, or a pharmaceutically acceptable salt of any of the foregoing, wherein:
Y² is OH, (═O), halo, triphenylphosphonium salt, or sulfonic ester;
n is an integer from 0 to 4; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
in the presence of one or more coupling reagents, to provide a compound of formula (I′-3):

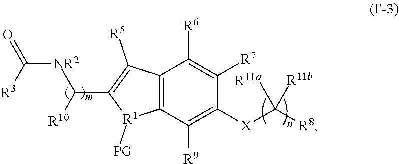

or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆ alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆ alkyl, C₁₋₆alkoxy, C₃₋₁₀cycloalkyl, or C₁₋₆haloalkyl, wherein
the C₁₋₆ alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
   the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
followed by,
(b) optionally contacting the compound of formula (I′-3) with a deprotecting agent;
to provide a compound of formula (I′).

Embodiment 69C. The process of embodiment 68C, wherein the protecting group is absent.

Embodiment 70C. The process of embodiment 68C, wherein the protecting group is a mesylate or tosylate.

Embodiment 71C. The process of any of embodiments 68C-70C, wherein the compound of formula (IV′-1) is an amine, the compound of formula (II′-2) is an aldehyde or ketone, and the reaction is a reductive amination.

Embodiment 72C. The process of any of embodiments 68C-70C, wherein the compound of formula (IV′-1) is a phenol, or thiol, the compound of formula (II′-2) is an alkyl halide or sulfonic ester and the reaction is an alkylation reaction.

Embodiment 73C. The process of embodiment 68C or 70C-72C, wherein the deprotecting agent comprises Mg.

Embodiment 74C. A process for preparing a compound of formula (I′), as defined in any of embodiments 1C-63C, or a stereoisomer or tautomer thereof, or a pharmaceutically acceptable salt of any of the foregoing, comprises:
reacting a compound of formula (III′-1):

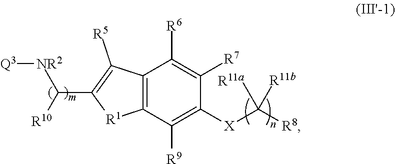

or a stereoisomer or tautomer thereof, or a pharmaceutically acceptable salt of any of the foregoing, wherein:
Q³ is H;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is independently —OH, oxo, —CN, halo, C₁₋₆ alkyl, C₁₋₆ alkoxy, C₁₋₆ haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆ alkyl, C₁₋₆ alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆ alkyl, or C₁₋₆ alkoxy;
with a compound of formula (III′-2):

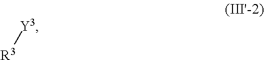

or a stereoisomer or tautomer thereof, or a pharmaceutically acceptable salt of any of the foregoing, wherein:
Y³ is —C(O)OH, or —C(O)-halo;
R³ is C₁₋₆alkyl, C₁₋₆haloalkyl, C₃₋₁₀cycloalkyl, —N(R⁴)₂, or 4-10 membered heterocyclyl, wherein
each R³ is optionally substituted with one or more R^{a}; and
each R^{a} is, independently at each occurrence, —OH, halo, C₁₋₆alkyl, C₁₋₆ haloalkyl, or C₃₋₁₀cycloalkyl;
in the presence of one or more coupling reagent, to provide a compound of formula (I′).

Embodiment 75C. The process of embodiment 74C, wherein the one or more coupling reagents comprises one or more reagents selected from the group consisting of HATU and DIEA.

Embodiment 76C. A pharmaceutical composition comprising (i) a compound of any of embodiments 1C-63C, or a stereoisomer or tautomer thereof, or a pharmaceutically acceptable salt of any of the foregoing, and (ii) one or more pharmaceutically acceptable excipients.

Embodiment 77C. A method of modulating SLC6A19 in a cell, comprising exposing the cell to a composition comprising an effective amount of a compound of any of embodiments 1C-63C, or a stereoisomer or tautomer thereof, or a pharmaceutically acceptable salt of any of the foregoing, or a pharmaceutical composition of embodiment 76C.

Embodiment 78C. A method of inhibiting SLC6A19 in a cell, comprising exposing the cell to a composition comprising an effective amount of a compound of any of embodiments 1C-63C, or a stereoisomer or tautomer thereof, or a pharmaceutically acceptable salt of any of the foregoing, or a pharmaceutical composition of embodiment 76C.

Embodiment 79C. A method of reducing systemic phenylalanine, tyrosine, glutamine, or glycine levels in an individual in need thereof, comprising administering to the individual an effective amount of a compound of any of embodiments 1C-63C, or a stereoisomer or tautomer thereof, or a pharmaceutically acceptable salt of any of the foregoing, or a pharmaceutical composition of embodiment 76C.

Embodiment 80C. A method of treating a SLC6A19-mediated disease, disorder, or condition in an individual in need thereof, comprising administering to the individual, a compound of any of embodiments 1C-63C, or a stereoisomer or tautomer thereof, or a pharmaceutically acceptable salt of any of the foregoing, or a pharmaceutical composition of embodiment 76C.

Embodiment 81C. The method of embodiment 80C, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

Embodiment 82C. The method of embodiment 80C or 81C, wherein the disease, disorder, or condition is selected from the group consisting of phenylketonuria (PKU), chronic kidney disease (CKD), metabolic syndrome, and metabolic diseases.

Embodiment 83C. The method of embodiment 80C, wherein the disease, disorder, or condition is associated with abnormal levels of amino acids.

Embodiment 84C. The method of embodiment 80C or 81C, wherein the disease, disorder, or condition is associated with a genetic defect in phenylalanine hydroxylase.

Embodiment 85C. The method of any of embodiments 80C-84C, wherein an effective amount of the compound is administered.

Embodiment 86C. The method of any of embodiments 80C-85C, wherein the individual is a human.

Embodiment 87C. A kit, comprising (i) a compound of any of embodiments 1C-63C, or a stereoisomer or tautomer thereof, or a pharmaceutically acceptable salt of any of the foregoing, or a pharmaceutical composition of embodiment 76C, and (ii) instructions for use in treating an SLC6A19-mediated disease, disorder, or condition in an individual in need thereof.

Embodiment 88C. The kit of embodiment 87C, wherein the disease, disorder, or condition is associated with abnormal levels of amino acids.

Embodiment 89C. The kit of embodiment 87C, or embodiment 88C, wherein the disease, disorder, or condition is associated with a genetic defect in phenylalanine hydroxylase.

Embodiment 90C. The kit of embodiment 87C, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

Embodiment 91C. The kit of embodiment 87C, wherein the individual has a genetic defect in phenylalanine hydroxylase.

### EXAMPLES

The following synthetic reaction schemes, which are detailed in the Schemes and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

In some embodiments, provided herein is a process for preparing a compound of formula (I′), or a stereoisomer or tautomer thereof, or a pharmaceutically acceptable salt of any of the foregoing according to any of Scheme 1-Scheme 34.

### Synthetic Examples

As depicted in the Schemes and Examples below, in certain exemplary embodiments, compounds of formula (I), or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

### Schemes

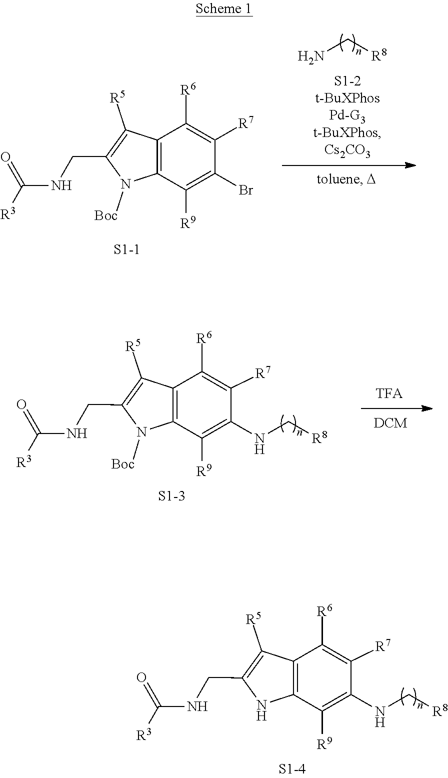

Compounds of formula S1-4 can be prepared according to Scheme 1. Buchwald coupling of aryl bromide S1-1 with amine S1-2 using a palladium catalyst such as t-BuXPhos Pd-G3, a phosphine ligand such as t-BuXPhos, and a base such as Cs₂CO₃ in an aprotic solvent such as toluene gives amine S1-3. Removal of the Boc protecting group with a protic acid such as TFA in a solvent such as DCM gives compounds of formula S1-4.

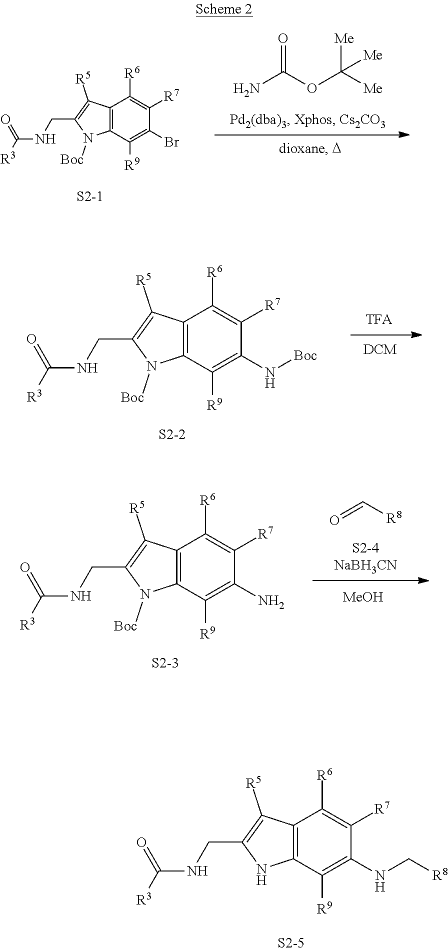

Compounds of formula S2-5 can be prepared according to Scheme 2. Buchwald coupling of aryl bromide S2-1 with tert-butyl carbamate using a palladium catalyst such as Pd₂(dba)₃, a phosphine ligand such as XPhos, and a base such as Cs₂CO₃ in an aprotic solvent such as dioxane gives amine S2-2. Removal of the Boc protecting group with a protic acid such as TFA in a solvent such as DCM gives amine S2-3. Reductive amination with aldehyde S2-4 using a reducing agent such as NaBH₃CN in a solvent such as MeOH gives compounds of formula S2-5.

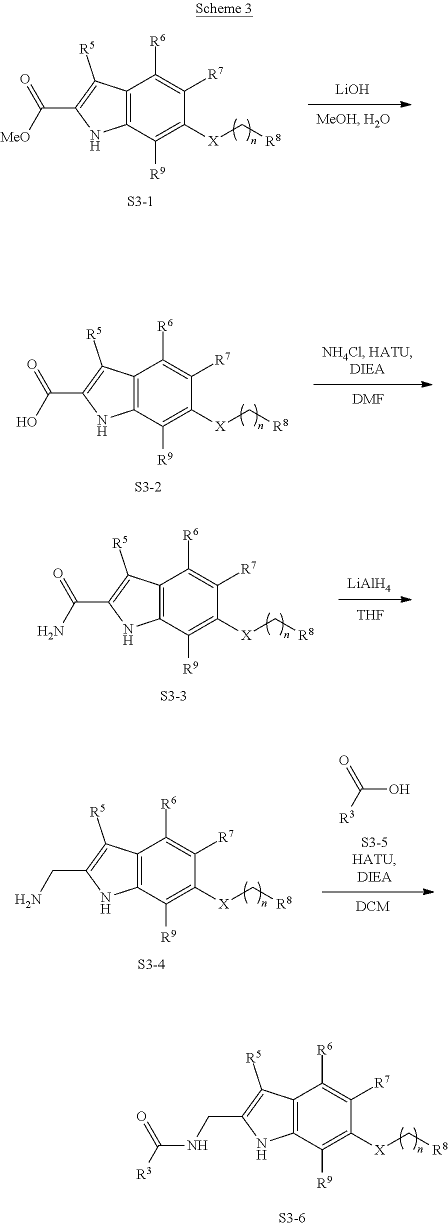

Compounds of formula S3-6 can be prepared according to Scheme 3. Hydrolysis of ester S3-1 with a base such as LiOH in solvents such MeOH and water affords acid S3-2. Amide coupling with ammonium chloride, a coupling reagent such as HATU, and a tertiary amine base such as DIEA in an aprotic solvent such as DMF gives amide S3-3, which can be reduced with a hydride reducing agent such as LiAlH₄ in an aprotic solvent such as THF to give primary amine S3-4. Amide coupling with carboxylic acid S3-5, a coupling reagent such as HATU, and a tertiary amine base such as DIEA in an aprotic solvent such as DCM gives compounds of formula S3-6.

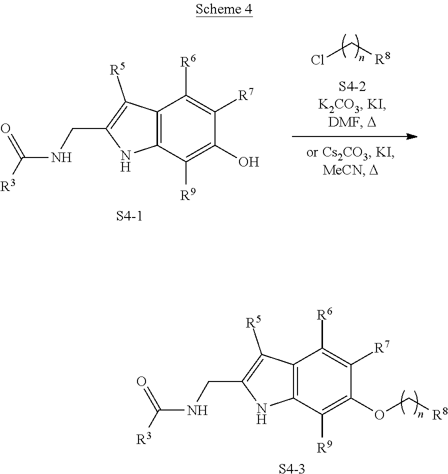

Compounds of formula S4-3 can be prepared according to Scheme 4. Alkylation of phenol S4-1 with alkyl chloride S4-2 using an inorganic base such as K₂CO₃ or Cs₂CO₃, and KI, in an aprotic solvent such as DMF or acetonitrile gives compounds of formula S4-3.

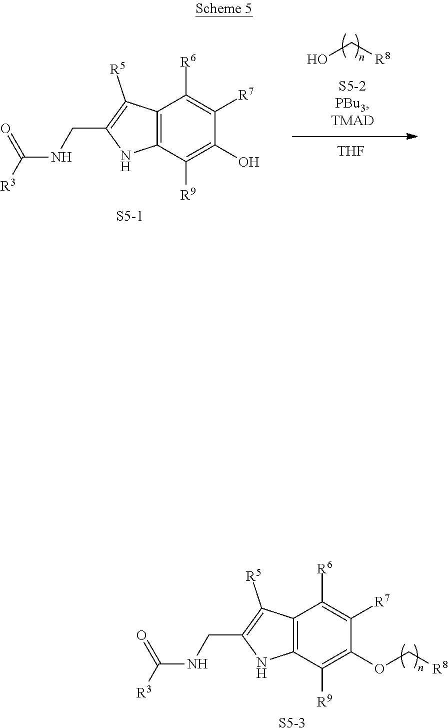

Compounds of formula S5-3 can be prepared according to Scheme 5. Mitsunobu reaction of phenol S5-1 with alcohol S5-2 using a trialkylphosphine such as PBu₃ and an azodicarboxylate ester such as tetramethylazodicarboxamide in an aprotic solvent such as THF gives compounds of formula S5-3, which may require additional deprotection or functional group manipulation using standard conditions to arrive at the final compounds.

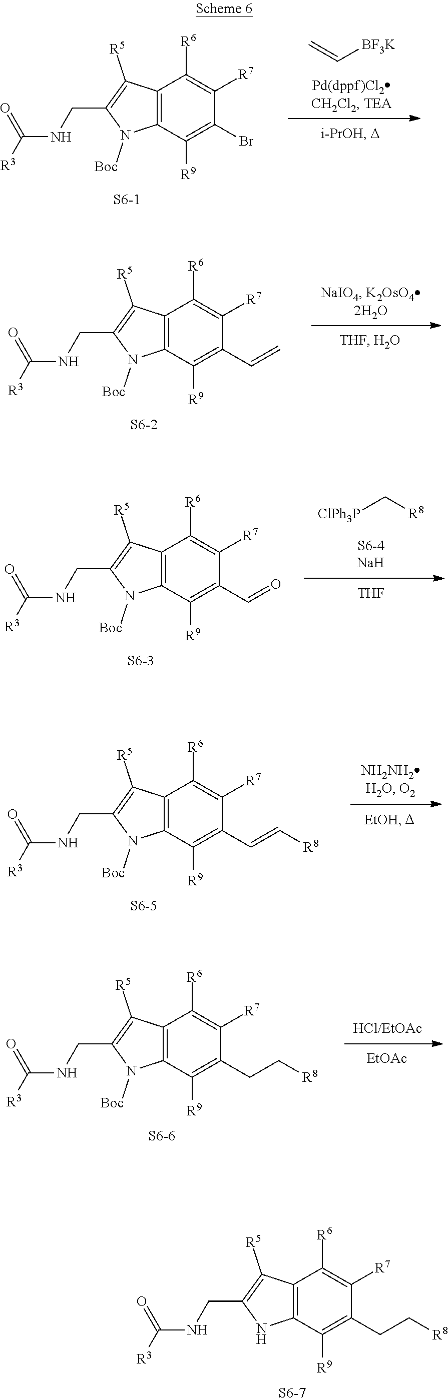

Compounds of formula S6-7 can be prepared according to Scheme 6. Suzuki coupling of aryl bromide S6-1 with potassium vinyltrifluoroborate using a palladium catalyst such as Pd(dppf)Cl₂·CH₂Cl₂ and a base such as TEA in a solvent such as isopropanol affords alkene S6-2. Oxidative cleavage with potassium osmate and sodium periodate in solvents such as THF and water gives aldehyde S6-3. Subsequent Wittig reaction with triphenylphosphonium salt S6-4 and a base such as NaH in an aprotic solvent such as THF affords S6-5 which can be reduced with hydrazine and oxygen in a solvent such as ethanol. Removal of the Boc protecting group with an acid such as HCl in a solvent such as EtOAc gives compounds of formula S6-7.

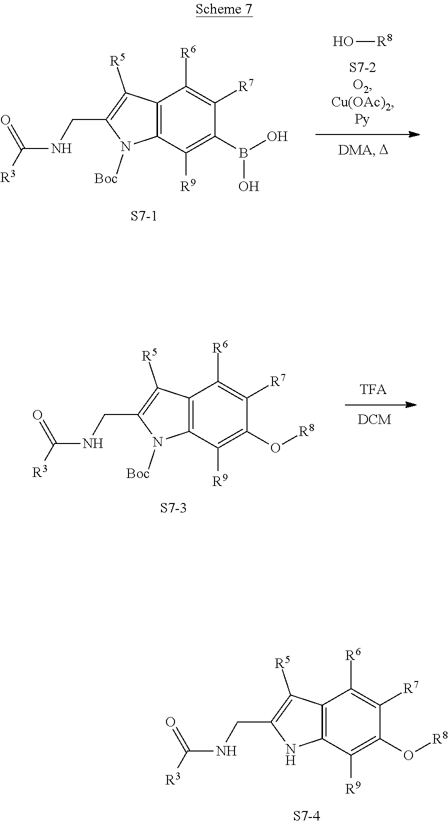

Compounds of formula S7-4 can be prepared according to Scheme 7. Ullmann-type coupling of a boronic acid such as S7-1 with an alcohol such as S7-2 under an oxygen atmosphere, in the presence of copper (II) acetate and pyridine, generates S7-3. Boc-group removal using a protic acid such as trifluoroacetic acid in an aprotic solvent such as DCM provides compounds of formula S7-4.

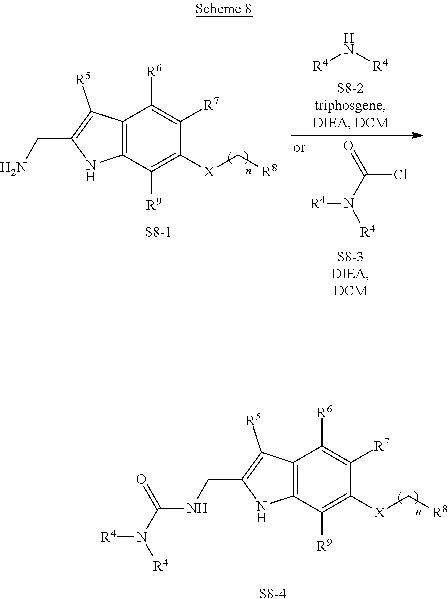

Compounds of formula S8-4 can be prepared with the two approaches shown in Scheme 8. Treatment of an amine such as S8-1 with triphosgene and a tertiary amine base such as DIEA, followed by reaction with a second amine such as S8-2, gives compounds of formula S8-4. Alternatively, amine S8-1 can be coupled with a carbamoyl chloride such as S8-3 in the presence of a tertiary amine base as DIEA to give compounds of formula S8-4, which may require additional deprotection or functional group manipulation using standard conditions to arrive at the final compounds.

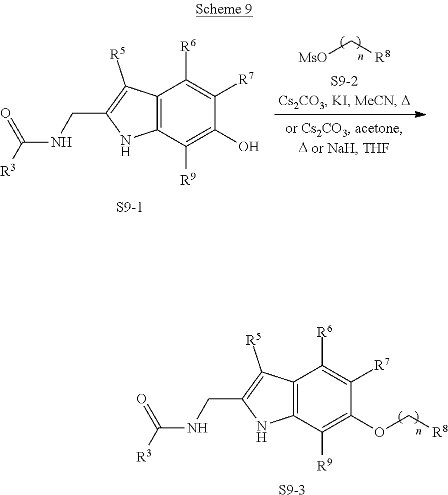

Compounds of formula S9-3 can be prepared according to Scheme 9. Heating indole S9-1 with an alkyl sulfonate ester such as S9-2 in the presence of potassium iodide and cesium carbonate in an aprotic solvent such as acetonitrile gives compounds of formula S9-3. Alternative conditions to couple S9-1 with S9-2 involve use of cesium carbonate in hot acetone. Additional alternative reaction conditions involve the use of sodium hydride as a base in THF.

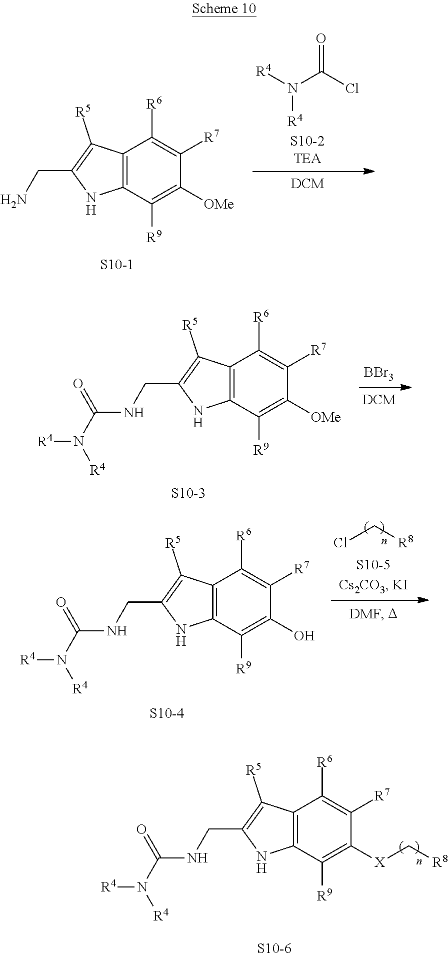

Compounds of formula S10-6 can be prepared according to Scheme 10. Amine S10-1 is converted to urea S10-3 upon reaction with carbamoyl chloride S10-2 using triethyl amine as a base. Methyl ether cleavage using a Lewis acid such as BBr₃ in an aprotic solvent such as DCM gives S10-4. Alkylation of S10-4 with an alkyl halide such as S10-5 in the presence of cesium carbonate and potassium iodide in DMF at elevated temperature gives compounds of formula S10-6.

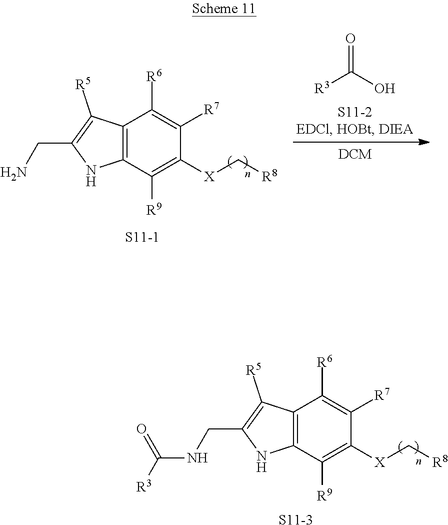

Compounds of formula S11-3 may be prepared according to Scheme 11. Coupling of amine S11-1 with a carboxylic acid such as S11-2 in the presence of EDCI, HOBt and a tertiary amine base such as DIEA in an aprotic solvent such as DCM gives compounds of formula S11-3.

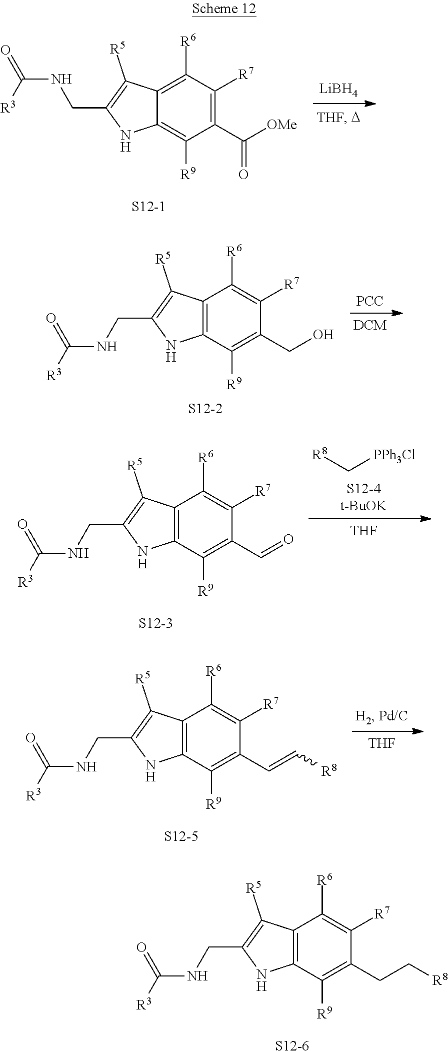

Compounds of formula S12-6 can be prepared according to Scheme 12. Reduction of ester S12-1 with lithium borohydride in THF gives alcohol S12-2. Treatment of S12-2 with an oxidizing agent such as PCC in DCM gives aldehyde S12-3. Reaction of S12-3 with an ylide derived from a phosphonium salt such as S12-4 and potassium tert-butoxide base gives olefin S12-5. Hydrogenation of S12-5 in the presence of palladium on carbon gives compounds of formula S12-6.

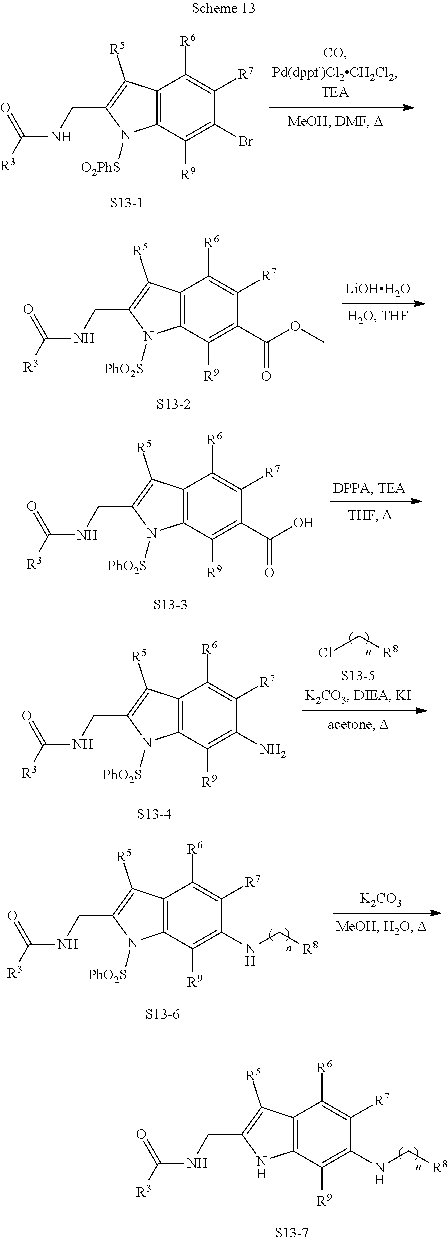

Compounds of formula S13-7 can be prepared according to Scheme 13. Heating indole S13-1 with a catalyst such as Pd(dppf)Cl₂·CH₂Cl₂, a tertiary amine base such as TEA, in DMF and MeOH under a CO atmosphere gives methyl ester S13-2. Ester hydrolysis gives acid S13-3, which undergoes a Curtius rearrangement upon heating with diphenylphosphoryl azide and TEA to give amine S13-4. Alkylation of S13-4 with an alkyl halide such as S13-5 can be achieved by heating in the presence of potassium carbonate, DIEA, and potassium iodide in acetone to give S13-6. Cleavage of the indole protecting group occurs upon heating S13-6 with potassium carbonate in a mixed solvent of methanol and water to give compounds of formula S13-7.

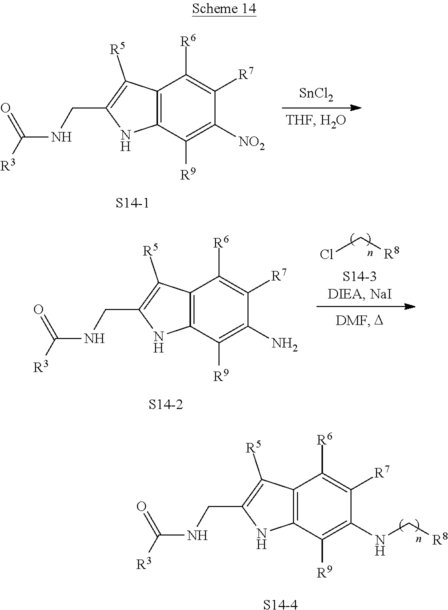

Compounds of formula S14-4 can be prepared according to Scheme 14. Nitro group reduction under the action of tin (II) chloride in THF and water gives S14-2. Alkylation of amine S14-2 with alkyl halide S14-3 occurs upon heating with DIEA and sodium iodide in an aprotic solvent such as DMF to give compounds of formula S14-4.

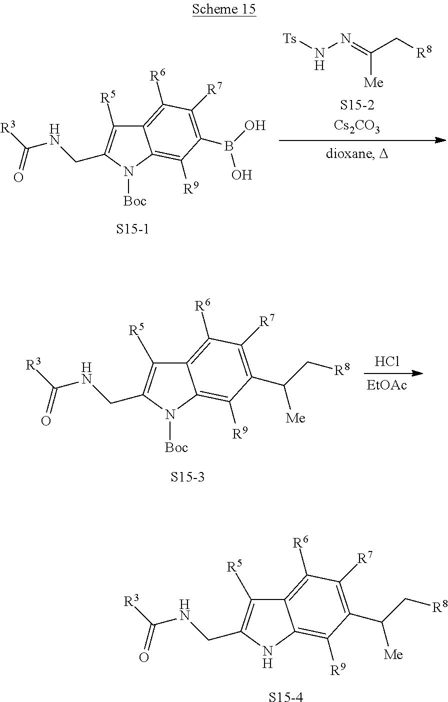

Compounds of formula S15-4 can be prepared according to Scheme 15. Heating a mixture of boronic acid S15-1, a tosyl hydrazone such as S15-2, and a base such as cesium carbonate in dioxane gives S15-3. Boc group cleavage occurs upon treatment with a protic acid such as HCl in an aprotic solvent such as EtOAc to give compounds of formula S15-4.

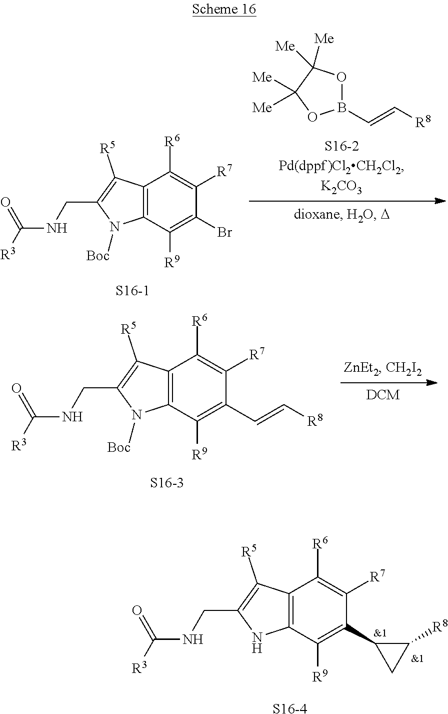

Compounds of formula S16-4 can be prepared according to Scheme 16. Suzuki coupling of S16-1 with a boronate ester such as S16-2 with a catalyst such as Pd(dppf)Cl₂·CH₂Cl₂ and a base such as potassium carbonate in aqueous dioxane gives olefin S16-3. Cyclopropanation occurs upon treatment with diethyl zinc and diiodomethane in DCM to give compounds of formula S16-4.

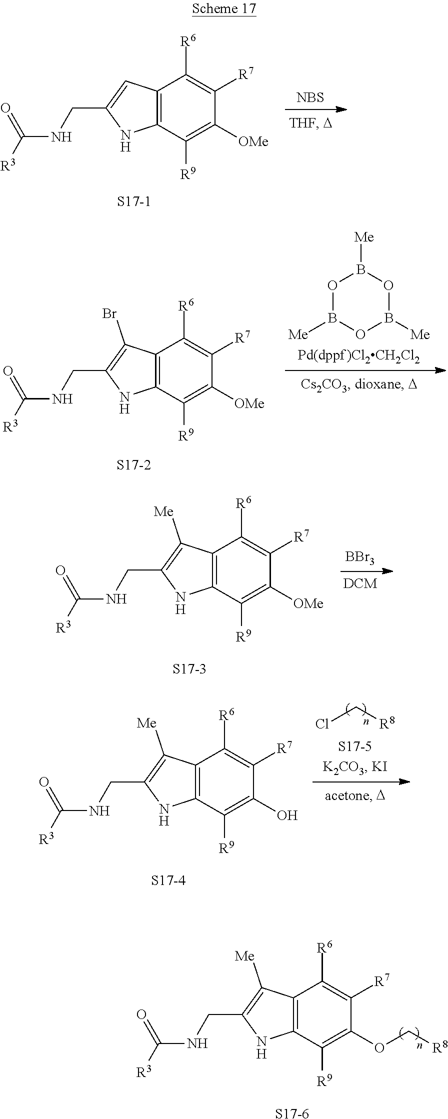

Compounds of formula S17-6 can be prepared according to Scheme 17. Treatment of indole S17-1 with NBS in hot THF gives S17-2. Suzuki coupling with trimethylboroxine with a catalyst such as Pd(dppf)Cl₂·CH₂Cl₂ and a base such as cesium carbonate in dioxane gives S17-3. Methyl ether cleavage upon treatment with BBr₃ gives S17-4. Alkylation occurs upon heating S17-4 in a mixture of alkyl halide S17-5, potassium carbonate, and potassium iodide in acetone to give compounds of formula S17-6.

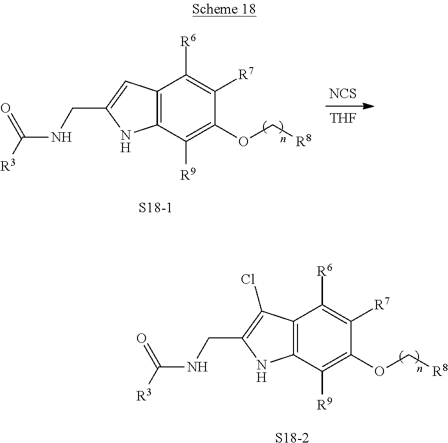

Compounds of formula S18-2 can be prepared according to Scheme 18. Treatment of indole S18-1 with N-chlorosuccinimide in THF gives compounds of formula S18-2.

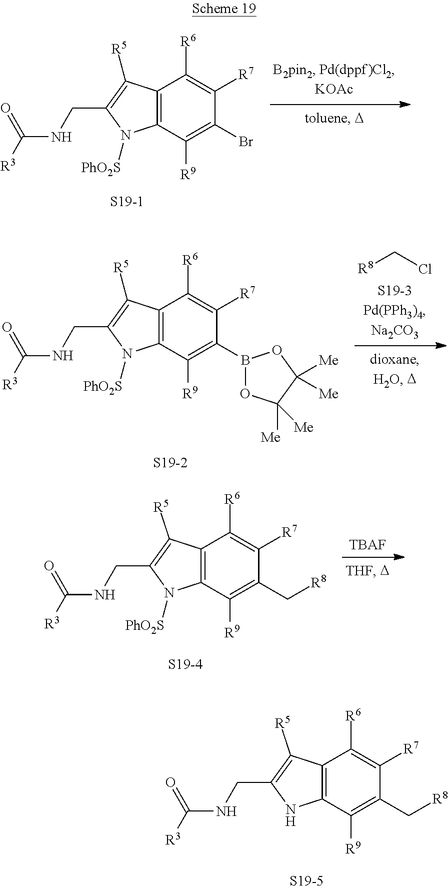

Compounds of formula S19-5 may be prepared according to Scheme 19. Heating a mixture of indole S19-1, bis(pinacolato)diboron, a palladium catalyst such as Pd(dppf)Cl₂·CH₂Cl₂, and potassium carbonate in aqueous dioxane gives boronate ester S19-2. Suzuki coupling of S19-2 and an alkyl halide such as S19-3, using a palladium catalyst such as palladium-tetrakis(triphenylphosphine) and a base such as sodium carbonate in aqueous dioxane gives S19-4. Cleavage of the benzenesulfonamide protecting group occurs upon heating with TBAF in THF to give compounds of formula S19-5.

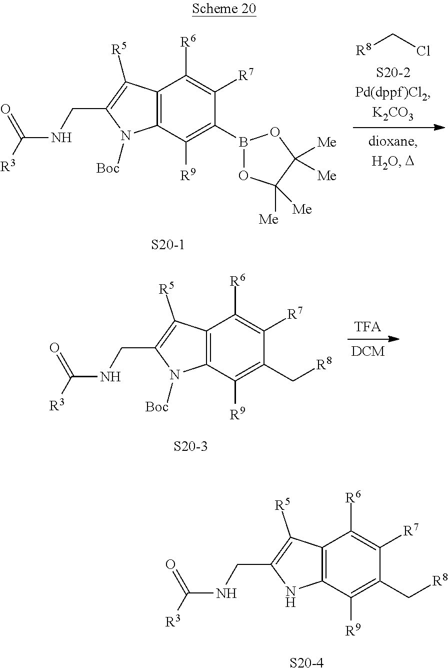

Compounds of formula S20-4 can be prepared according to Scheme 20. Boc-protected indole S20-1 may undergo Suzuki coupling with alkyl chloride S20-2 according to conditions described in Scheme 19, using a base such as potassium carbonate to give S20-3. Boc group cleavage occurs upon treatment with TFA in an aprotic solvent such as DCM to give compounds of formula S20-4.

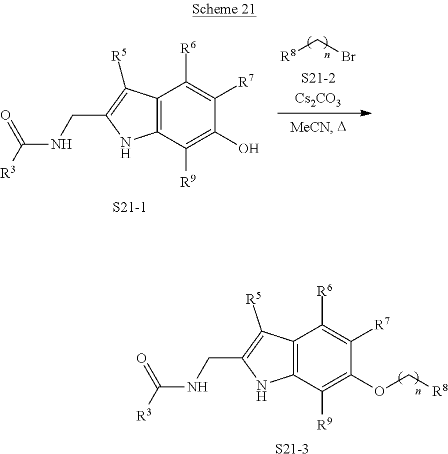

Compounds of formula S21-3 can be prepared according to Scheme 21. Alkylation of indole S21-1 with an alkyl halide such as S21-2 occurs upon heating with cesium carbonate in acetonitrile to give compounds of formula S21-3.

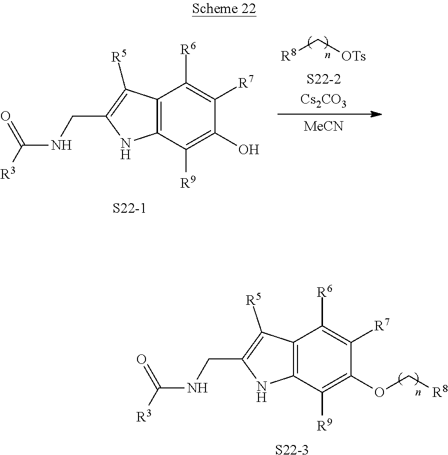

Compounds of formula S22-3 can be prepared according to Scheme 22. Reaction of indole S22-1 with sulfonate ester such as S22-2 in the presence of cesium carbonate gives compounds of formula S22-3.

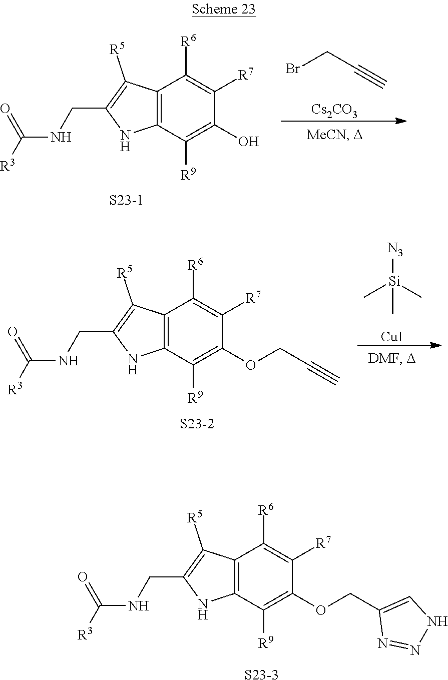

Compounds of formula S23-3 can be prepared according to Scheme 23. Heating indole S23-1 with a propargyl halide such as propargyl bromide in the presence of cesium carbonate gives alkyne S23-2. A copper iodide-mediated reaction with azidotrimethylsilane gives compounds of formula S23-3.

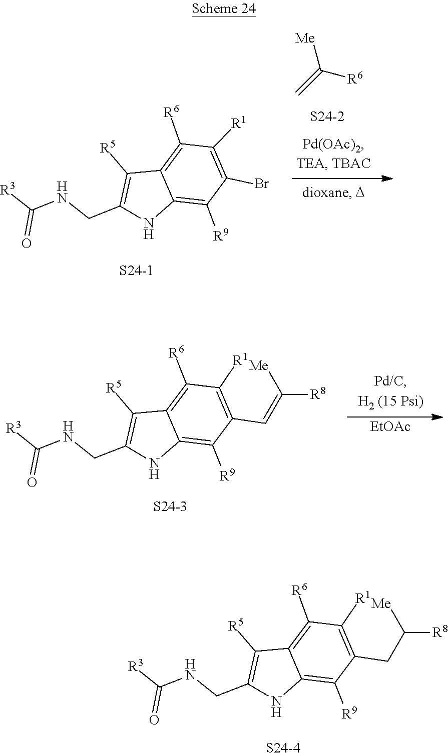

Compounds of formula S24-4 can be prepared according to Scheme 24. Heating a mixture of S24-1 and olefin S24-2 with palladium acetate, triethylamine, and tetrabutylammonium chloride gives olefin S24-3. Alkene hydrogenation with a catalyst such as Pd/C in EtOAc solvent gives compounds of formula S24-4.

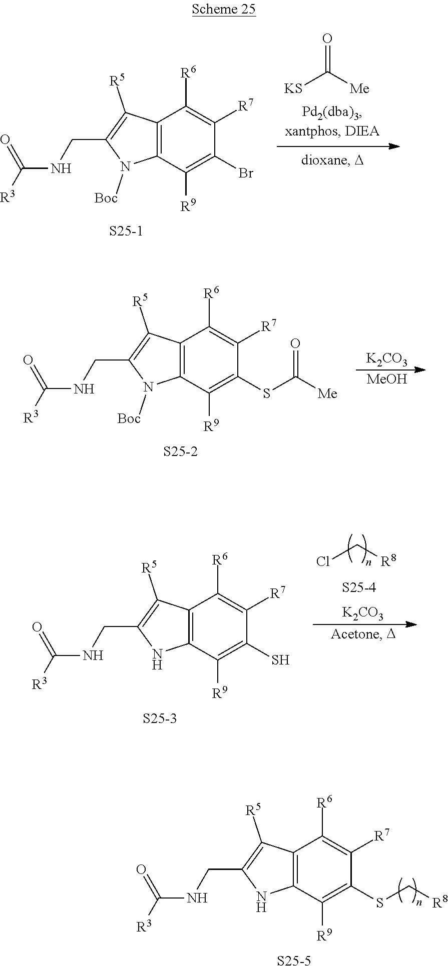

Compounds of formula S25-5 can be prepared according to Scheme 25. Coupling of potassium thioacetate with indole S25-1 using a catalyst derived from tris(dibenzylideneacetone)dipalladium(0) and Xantphos, with DIEA as base, gives thiol ester S25-2. Thioester cleavage upon reaction of S25-2 with methanol and potassium carbonate gives S25-3. Alkylation with alkyl halide S25-4 under conditions previously described gives compounds of formula S25-5.

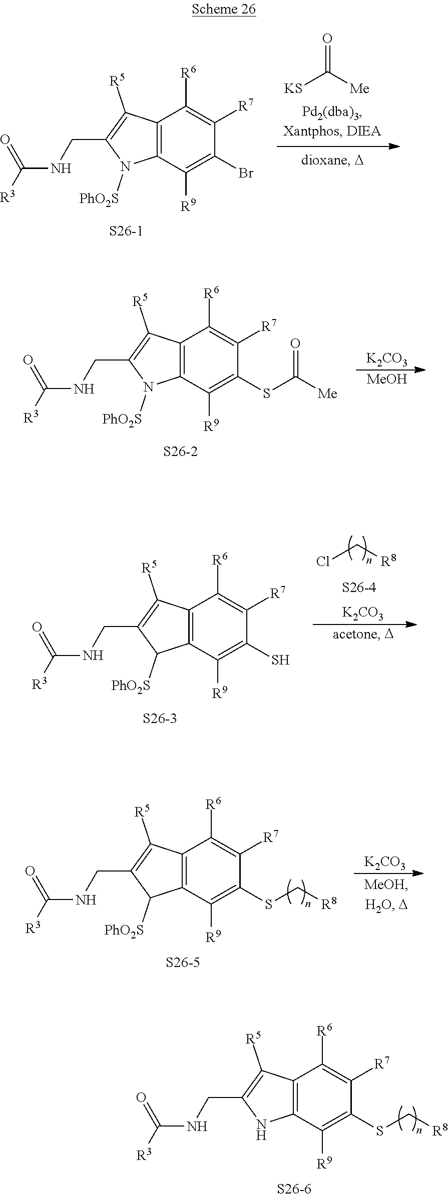

Compounds of formula S26-6 can be prepared according to Scheme 26. Coupling of potassium thioacetate with indole S26-1 using a catalyst derived from tris(dibenzylideneacetone)dipalladium(0) and Xantphos, with DIEA as base, gives thiol ester S26-2. Thioester cleavage upon reaction of S26-2 with methanol and potassium carbonate gives S26-3. Alkylation with alkyl halide S26-4 under conditions previously described gives S26-5. Cleavage of the benzenesulfonamide protecting group occurs upon heating with potassium carbonate in methanol and water to give compounds of the formula S26-6.

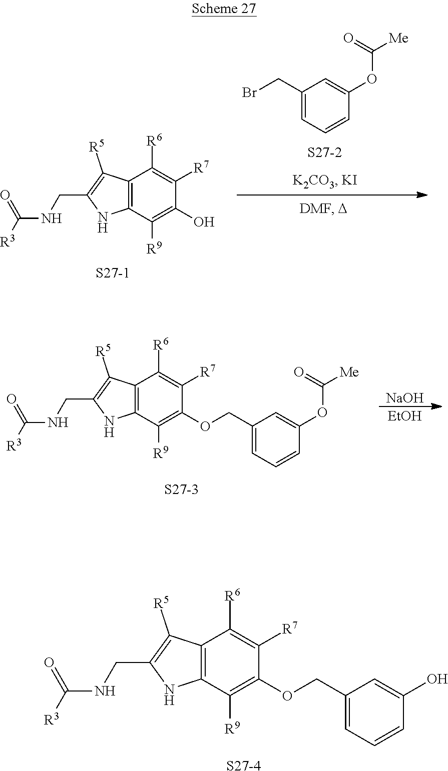

Compounds of formula S27-3 can be prepared according to Scheme 27. Alkylation of indole S27-1 with a benzylic bromide such as S27-2 under conditions previously described gives S27-3. Ester hydrolysis with NaOH in ethanol gives compounds of formula S27-4.

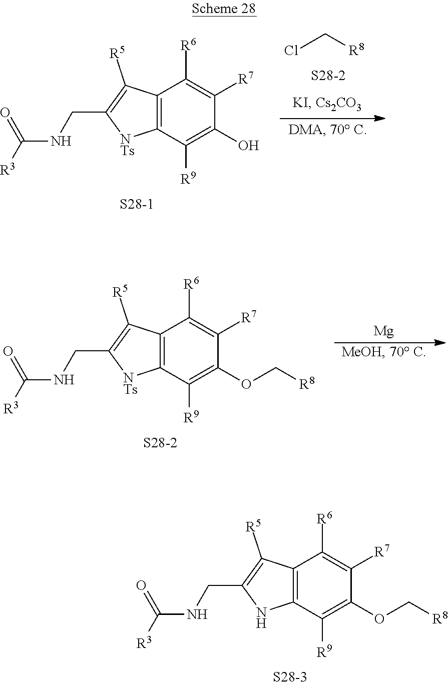

Compounds of formula S28-3 can be prepared according to Scheme 28. Heating a solution of indole S28-1 and a benzylic chloride such as S28-2 in DMA in the presence of potassium iodide and cesium carbonate gives S28-2. Removal of the N-tosyl group occurs upon heating with magnesium in methanol to give compounds of formula S28-3.

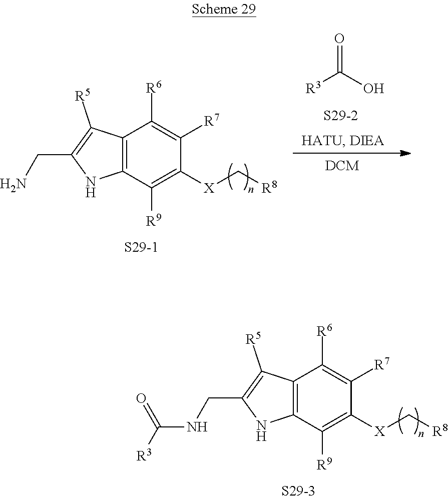

Compounds of formula S29-3 may be prepared according to Scheme 29. Coupling of amine S29-1 with a carboxylic acid such as S29-2 in the presence of HATU and a tertiary amine base such as DIEA in an aprotic solvent such as DCM gives compounds of formula S29-3.

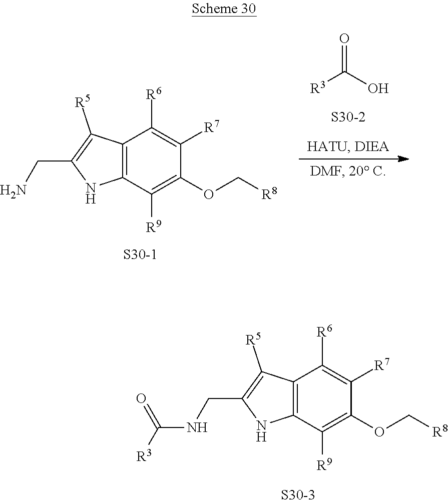

Compounds of formula S30-3 may be prepared according to Scheme 30. Coupling of amine S30-1 with a carboxylic acid such as S30-2 in the presence of HATU and a tertiary amine base such as DIEA in an aprotic solvent such as DMF gives compounds of formula S30-3, which may require additional deprotection or functional group manipulation using standard conditions to arrive at the final compounds.

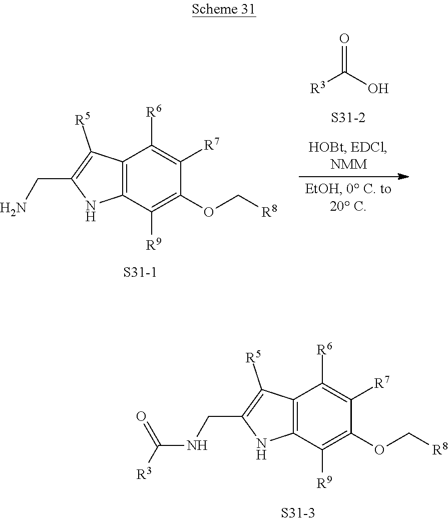

Compounds of formula S31-3 may be prepared according to Scheme 31. Coupling of amine S31-1 with a carboxylic acid such as S31-2 in the presence of HOBt, EDCI and a tertiary amine base such as NMM in a solvent such as EtOH gives compounds of formula S31-3, which may require additional deprotection or functional group manipulation using standard conditions to arrive at the final compounds.

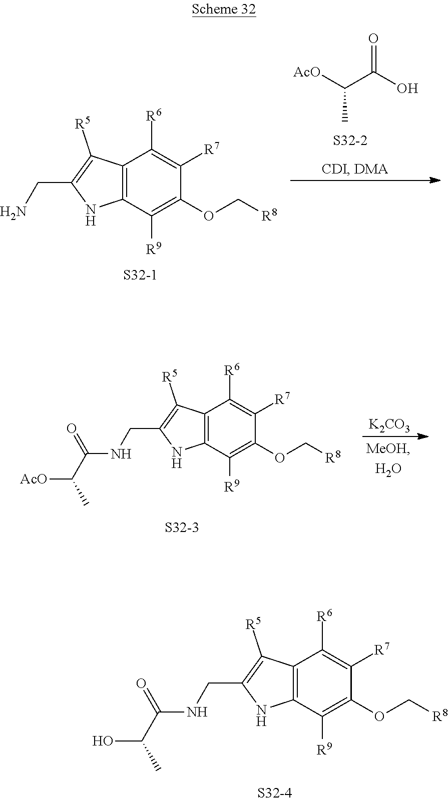

Compounds of formula S32-4 may be prepared according to Scheme 32. Coupling of amine S32-1 with a carboxylic acid such as S32-2 in the presence of CDI in a solvent such as DMA gives compounds of formula S32-3. Cleavage of the ester of S32-3 occurs upon treatment with potassium carbonate in a solvent such as methanol and water to give compounds of formula S32-4.

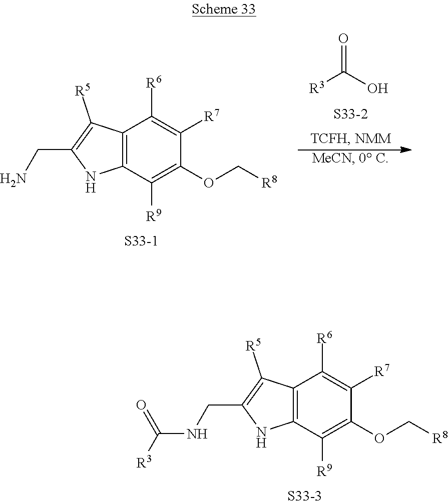

Compounds of formula S33-3 may be prepared according to Scheme 33. Coupling of amine S33-1 with a carboxylic acid such as S33-2 in the presence of TCFH and an amine base such as NMI in a solvent such as MeCN gives compounds of formula S33-3.

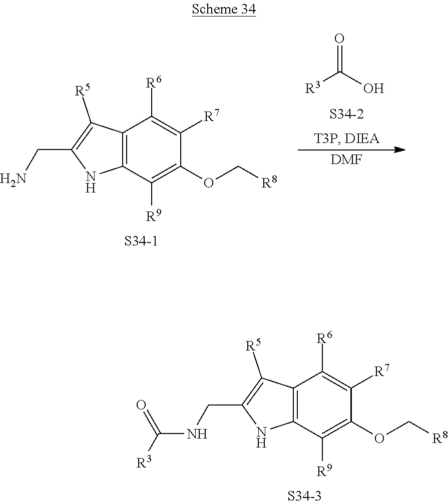

Compounds of formula S34-3 may be prepared according to Scheme 34. Coupling of amine S34-1 with a carboxylic acid such as S34-2 in the presence of T3P and an amine base such as DIEA in a solvent such as DMF gives compounds of formula S34-3, which may require additional deprotection or functional group manipulation using standard conditions to arrive at the final compounds.

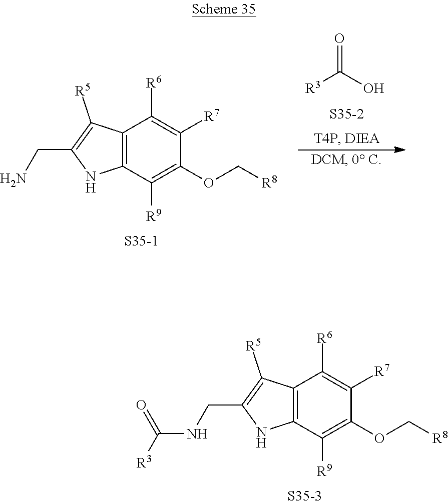

Compounds of formula S35-3 may be prepared according to Scheme 35. Coupling of amine S35-1 with a carboxylic acid such as S35-2 in the presence of T4P and an amine base such as DIEA in a solvent such as DCM gives compounds of formula S35-3, which may require additional deprotection or functional group manipulation using standard conditions to arrive at the final compounds.

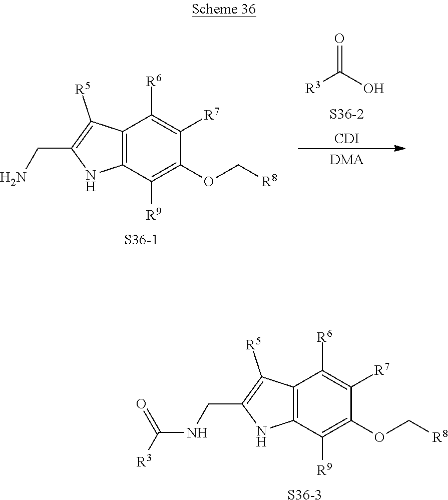

Compounds of formula S36-3 may be prepared according to Scheme 36. Coupling of amine S36-1 with a carboxylic acid such as S36-2 in the presence of CDI in a solvent such as DMA gives compounds of formula S36-3.

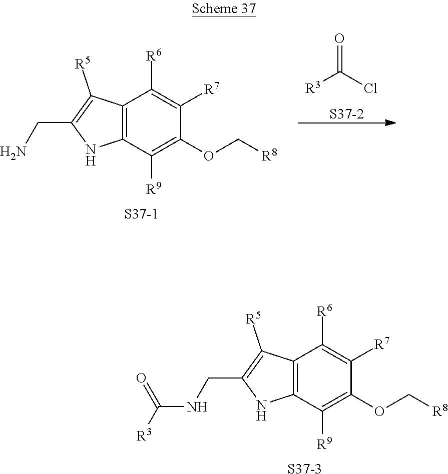

Compounds of formula S37-3 may be prepared according to Scheme 37. Coupling of amine S37-1 with an acid chloride such as S37-2 in the presence of a base such as DIEA in a solvent such as DCM gives compounds of formula S37-3.

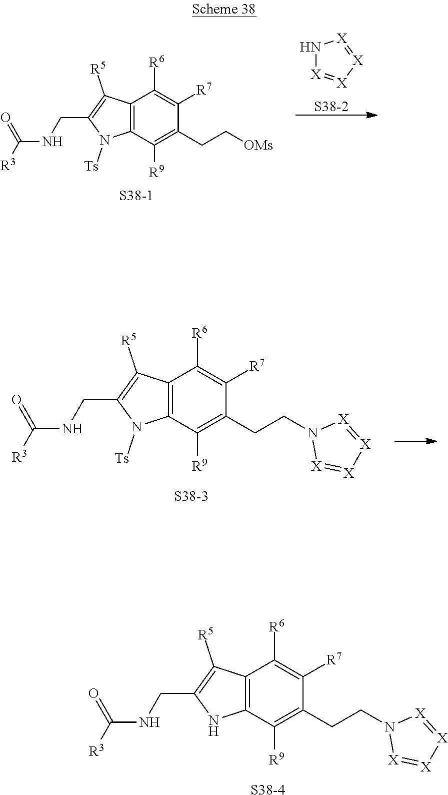

Compounds of formula S38-4 may be prepared according to Scheme 38. Treatment of an amide such as S38-1 with an aza heterocycle such as S38-2 in the presence of a base such as K₂CO₃ in a solvent such as DMF gives compounds of formula S38-3. Deprotection of the indole nitrogen with a reagent such as TBAF in a solvent such as THF affords compounds of the formula S38-4.

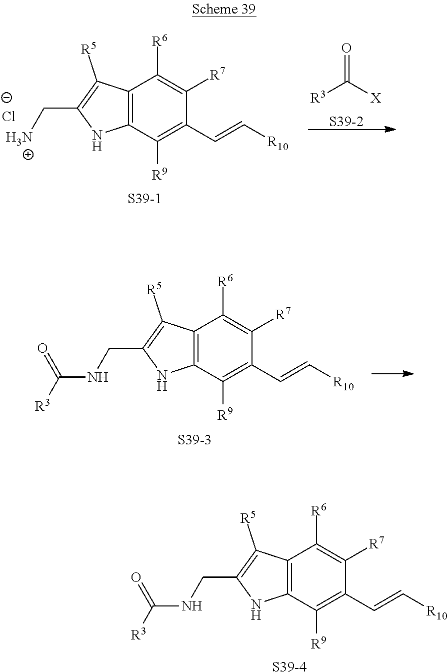

Compounds of formula S39-4 may be prepared according to Scheme 39. Treatment of an amine such as S39-1, which may be the free base or a salt form, with a reagent such as S39-2 in the presence of a reagent such as HATU and/or a base such as DIEA in a solvent such as DCM or THF gives compounds of formula S39-3. Hydrogenation of the olefin in the presence of a catalyst such as Pd/C in a solvent such as THF affords compounds of the formula S39-4.

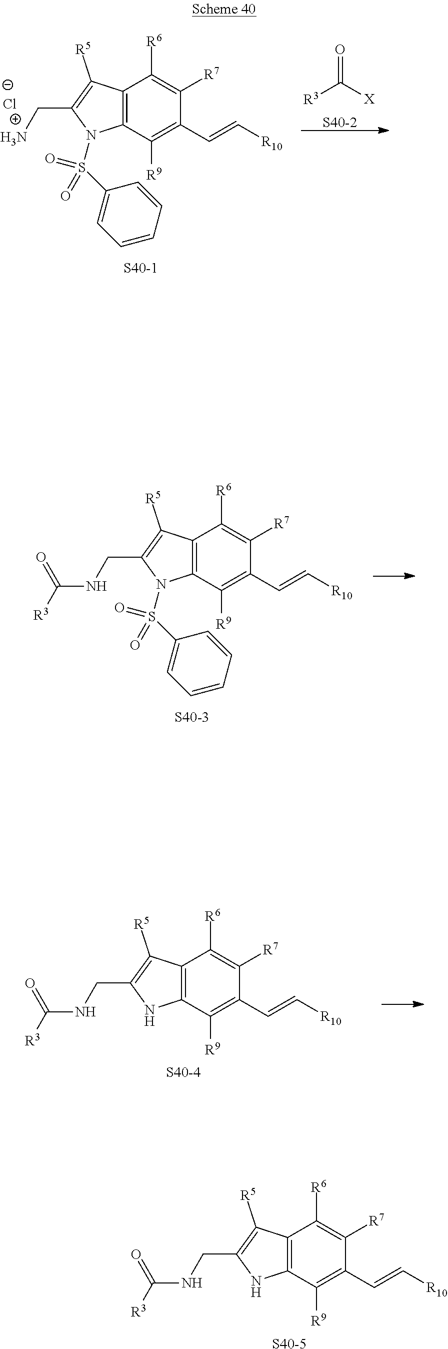

Compounds of formula S40-5 may be prepared according to Scheme 40. Treatment of an amine hydrochloride salt such as S40-1 with a reagent such as S40-2 in the presence of a reagent such as HATU and a base such as DIEA in a solvent such as DMF gives compounds of formula S40-3. Deprotection of the indole nitrogen using a reagent such as TBAF in a solvent such as THF gives compounds of the formula S40-4. Hydrogenation of the olefin in the presence of a catalyst such as Pd/C in a solvent such as THF affords compounds of the formula S40-5.

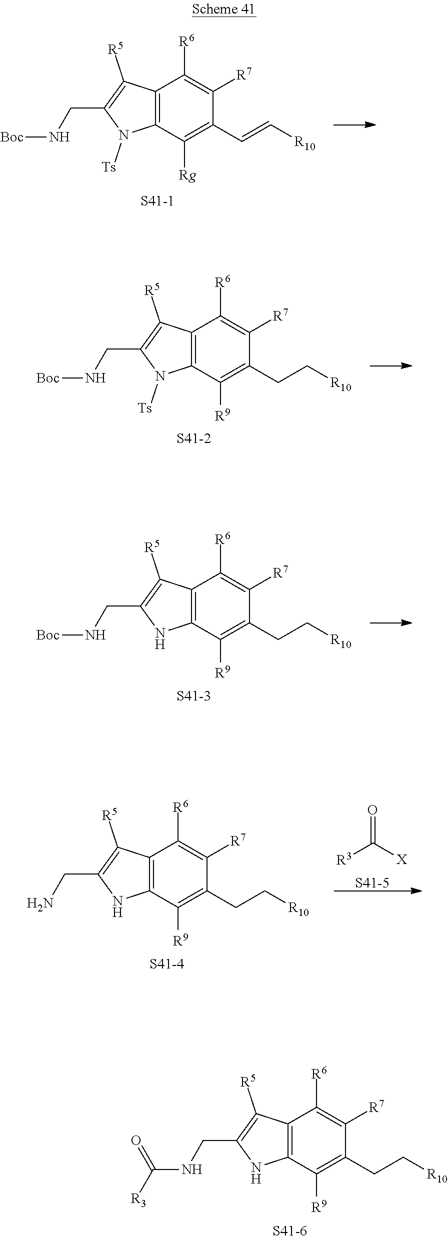

Compounds of formula S41-6 may be prepared according to Scheme 41. Hydrogenation of an olefin such as S41-1 in the presence of a catalyst such as Pd/C in a solvent such as THF gives compounds of formula S41-2. Deprotection of the indole nitrogen using a reagent such as TBAF in a solvent such as THF gives compounds of the formula S41-3. Deprotection of the amine with a reagent such as HCl in a solvent such as EtOAc gives compounds of the formula S41-4. Reaction of the amine with reagents such as S41-5 gives compounds of the formula S41-6.

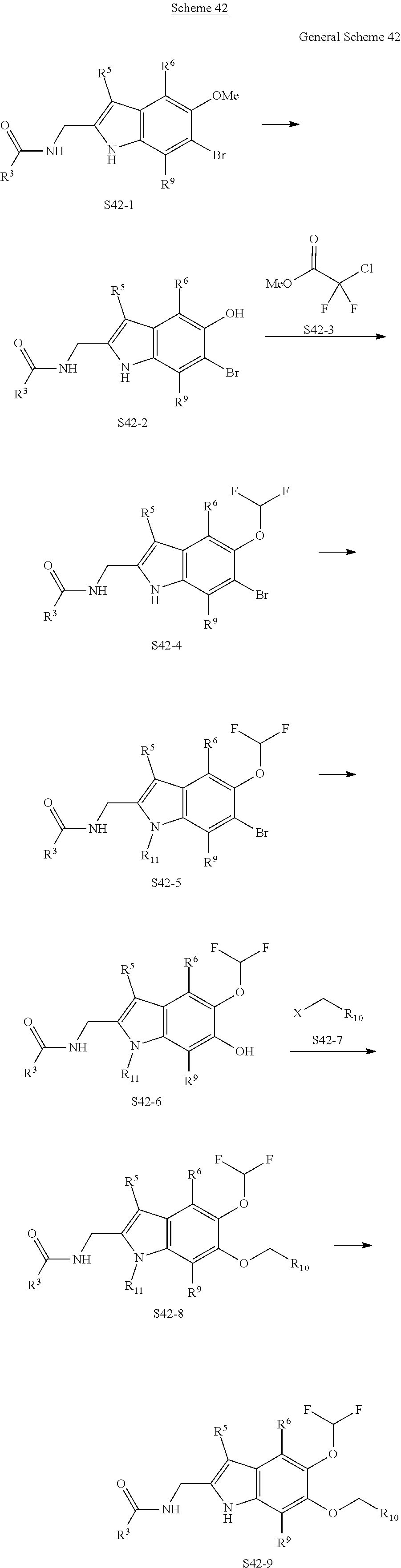

Compounds of formula S42-9 may be prepared according to Scheme 42. Demethylation of compounds such as S42-1 using reagents such as BBr₃ in a solvent such as DCM gives compounds of the formula S42-2. Installation of the difluoromethyl group using a reagent such as S42-3 gives compounds of the formula S42-4. Protection of the indole nitrogen using a reagent such as benzenesulfonyl chloride or tosyl chloride in a solvent such as DMA gives compounds of the formula S42-5. Installation of the hydroxyl group using a reagent such as KOH in the presence of a catalyst such as Pd₂(dba)₃, a ligand such as t-BuXPhos, a base such as Cs₂CO₃, and a mixed solvent system such as dioxane/water gives compounds of the formula S42-6. Alkylation of the hydroxyl group with a reagent such as S42-7 under Mitsunobu conditions using reagents such as PBu₃ and TMAD in a solvent such as THF or, alternatively, in the presence of a base such as Cs₂CO₃ and/or DIEA and a reagent such as KI in a solvent such as DMF gives compounds of the formula S42-8. Deprotection of the indole nitrogen using reagents such as TBAF in a solvent such as THF gives compounds of the formula S42-9.

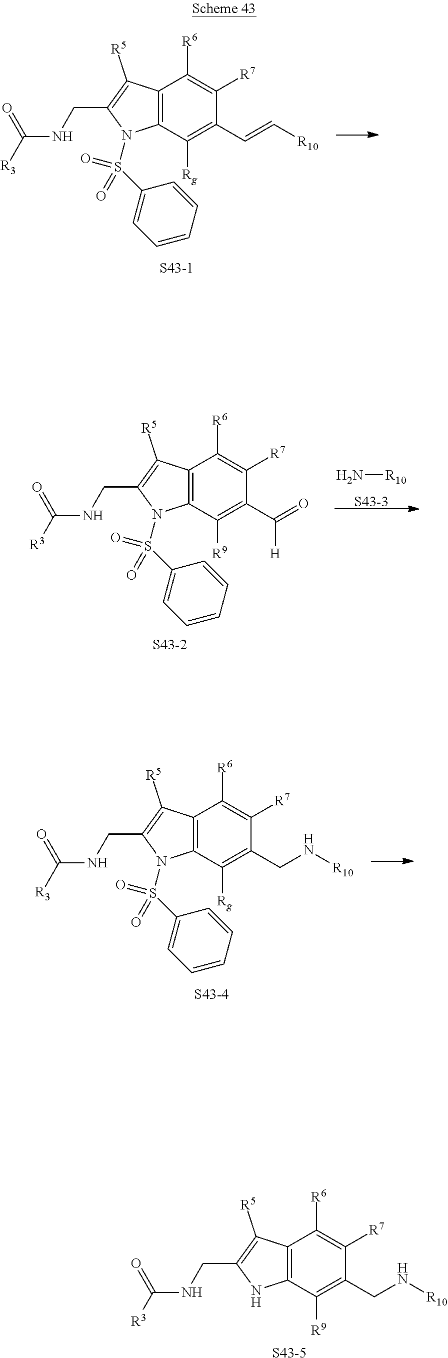

Compounds of formula S43-5 may be prepared according to Scheme 43. Treatment of compounds such as S43-1 with reagents such as i-PrMgCl·LiCl and DMF in a solvent such as THF gives compounds of the formula S43-2. Reductive amination using a reagent such as S43-3 in the presence of reagents such as Et₃SiH and TFA in a solvent system such as DCM and MeCN gives compounds of the formula S43-4. Deprotection of the indole nitrogen using a reagent such as KOH in a solvent system such as DMA and acetonitrile gives compounds of the formula S43-5.

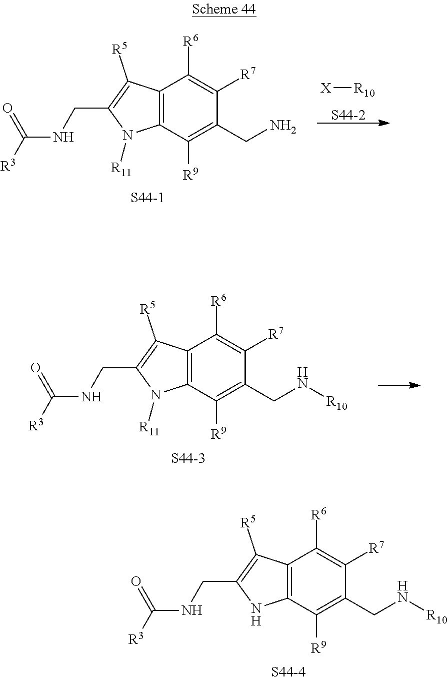

Compounds of formula S44-4 may be prepared according to Scheme 44. Reaction of protected indoles such as S44-1 with reagents such as S44-2, which may be a heterocycle bearing a leaving group such as bromo or nonaflate, under either cross coupling conditions using a catalyst such as Pd₂(dba)₃, a ligand such as BINAP, and a base such as t-BuONa in a solvent such as toluene or SnAr conditions using a solvent such as DMA gives compounds of the formula S44-3. Deprotection of the indole nitrogen using a reagent such as TBAF in a solvent such as THF gives compounds of the formula S44-4.

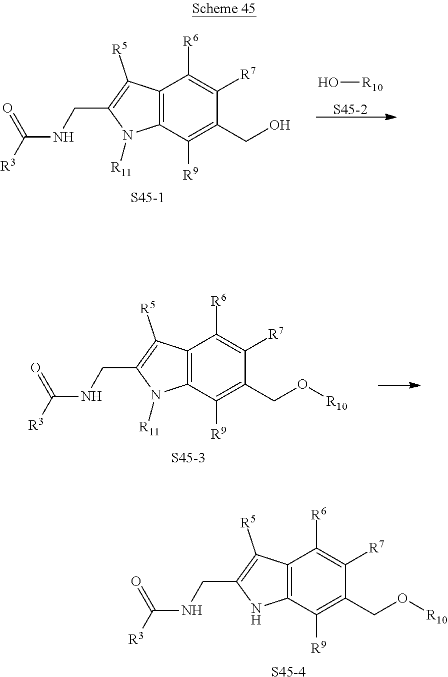

Compounds of formula S45-4 may be prepared according to Scheme 45. Reaction of protected indoles such as S45-1 with reagents such as S44-2 under Mitsunobu conditions using reagents such as PBu₃ and TMAD in a solvent such as THF gives compounds of the formula S45-3. Deprotection of the indole nitrogen using a reagent such as TBAF in a solvent such as THF gives compounds of the formula S45-4.

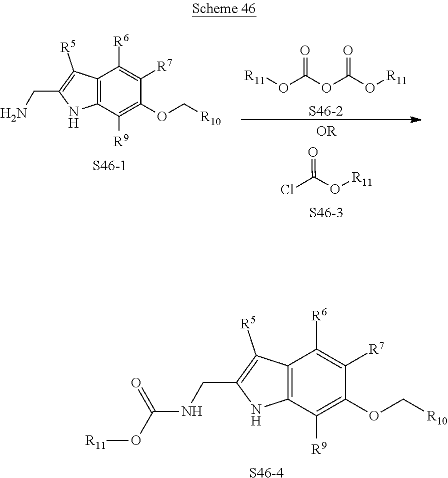

Compounds of formula S46-4 may be prepared according to Scheme 46. Reaction of compounds such as S46-1 with reagents such as S46-2 or S46-3 in the presence of bases such as DIEA, TEA, and/or NaHCO₃ in a solvent such system such as DCM or THF/H₂O gives compounds of the formula S46-4.

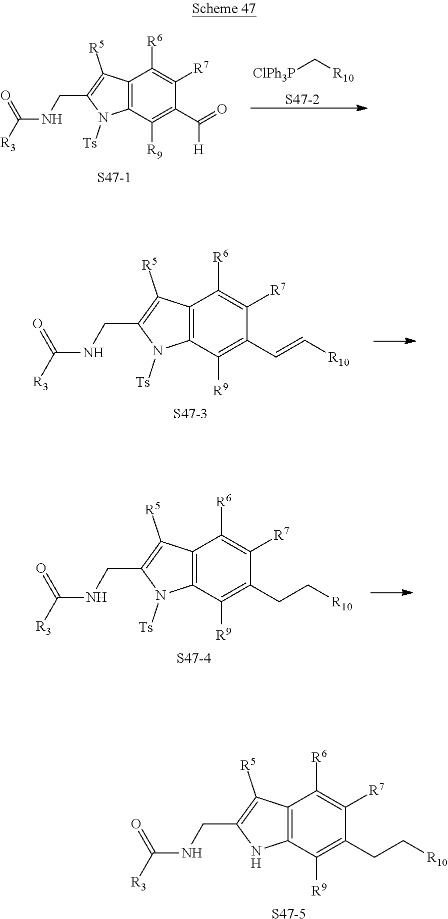

Compounds of formula S47-5 may be prepared according to Scheme 47. Reaction of compounds such as S47-1 with reagents such as S47-2 in the presence of reagents such as sodium in a solvent such as THF gives compounds of the formula S47-3. Hydrogenation of the olefin in the presence of a catalyst such as Pd/C in a solvent such as THF gives compounds of the formula S47-4. Deprotection of the indole nitrogen using a reagent such as TBAF in a solvent such as THF gives compounds of the formula S47-5.

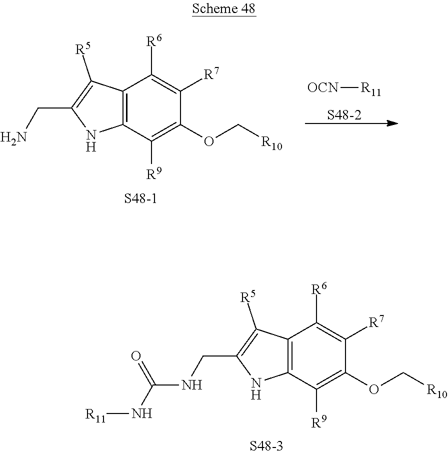

Compounds of formula S48-3 may be prepared according to Scheme 48. Reaction of compounds such as S48-1 with reagents such as S48-2 in the presence of a base such as DIEA in a solvent such as DCM gives compounds of the formula S48-3.

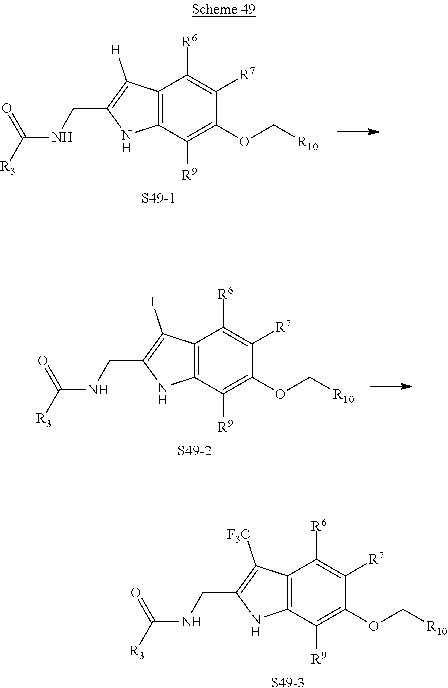

Compounds of formula S49-3 may be prepared according to Scheme 49. Reaction of compounds such as S49-1 with reagents such as NIS in a solvent such as DCM gives compounds of the formula S49-2. Treatment with reagents such as [Ph₂SCF₃]⁺[OTf]⁻ and copper in a solvent such as DMF gives compounds of the formula S49-3.

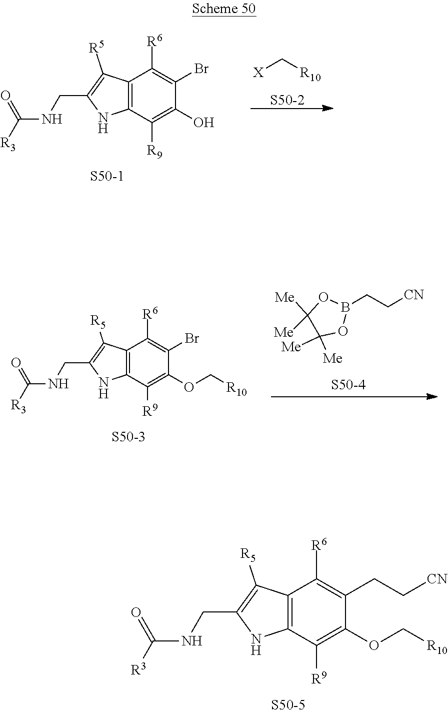

Compounds of formula S50-5 may be prepared according to Scheme 50. Reaction of compounds such as S50-1 with reagents such as S50-2 in the presence of a base such as K₂CO₃ and a reagent such as KI in a solvent such as DMF gives compounds of the formula S50-3. Cross coupling with reagents such as S50-4 under the action of a catalyst such as CataCXiumPdG2 and a base such as Cs₂CO₃ in a solvent system such as 1,4-dioxane/water gives compounds of the formula S50-5.

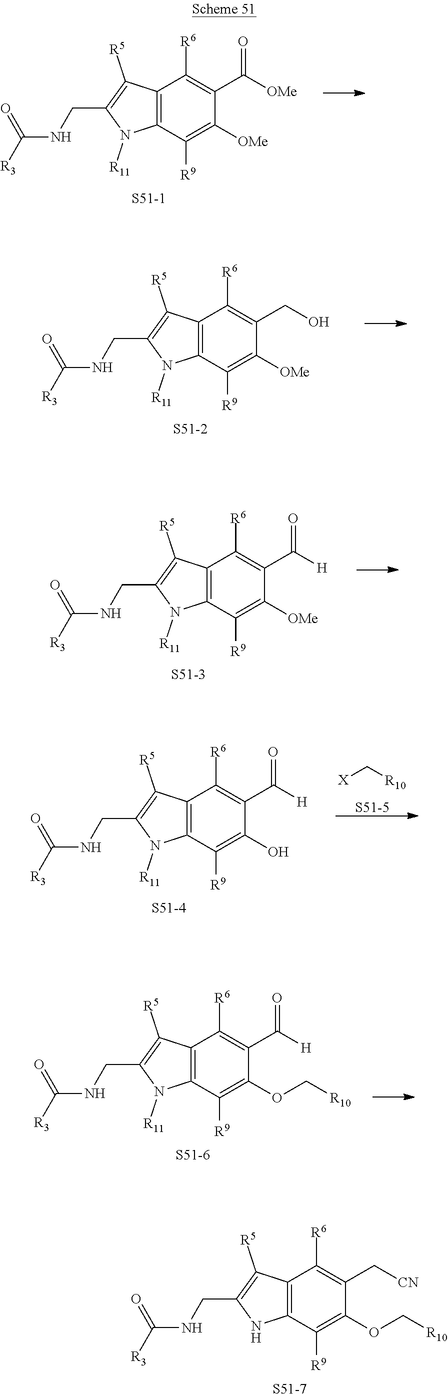

Compounds of formula S51-7 may be prepared according to Scheme 51. Reaction of compounds such as S51-1 with reagents such as LiBH₄ in a solvent such as THF gives compounds of the formula S51-2. Oxidation of the alcohol with reagents such as PCC in solvents such as DCM gives compounds of the formula S51-3. Demethylation using reagents such as BBr₃ in a solvent such as DCM gives compounds of the formula S51-4. Alkylation of the alcohol using reagents such as S51-5 in the presence of a base such as K₂CO₃ and/or DIEA and a reagent such as KI in a solvent such as DMF gives compounds of the formula S51-6. Treatment with a reagent such as TosMIC in the presence of a base such as t-BuOK in a solvent such as THF followed by heating with a reagent such as MeOH gives compounds of the formula S51-7.

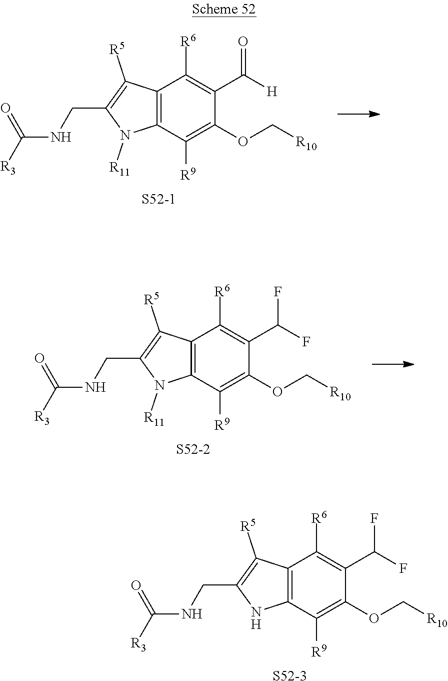

Compounds of formula S52-3 may be prepared according to Scheme 52. Reaction of compounds such as S52-1 with reagents such as BAST in a solvent such as DCM gives compounds of the formula S52-2. Deprotection of the indole nitrogen using a reagent such as TBAF in a solvent such as THF gives compounds of the formula S52-3.

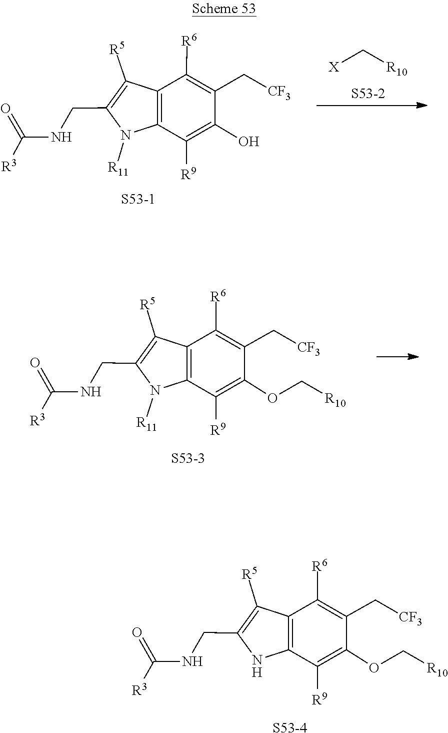

Compounds of formula S53-4 may be prepared according to Scheme 53. Alkylation of compounds such as S53-1 with reagents such as S53-2 in the presence of a base such as Cs₂CO₃ and a reagent such as KI in a solvent such as acetonitrile gives compounds of the formula S53-3. Deprotection of the indole nitrogen using a reagent such as KOH in a solvent system such as EtOH/water gives compounds of the formula S53-4.

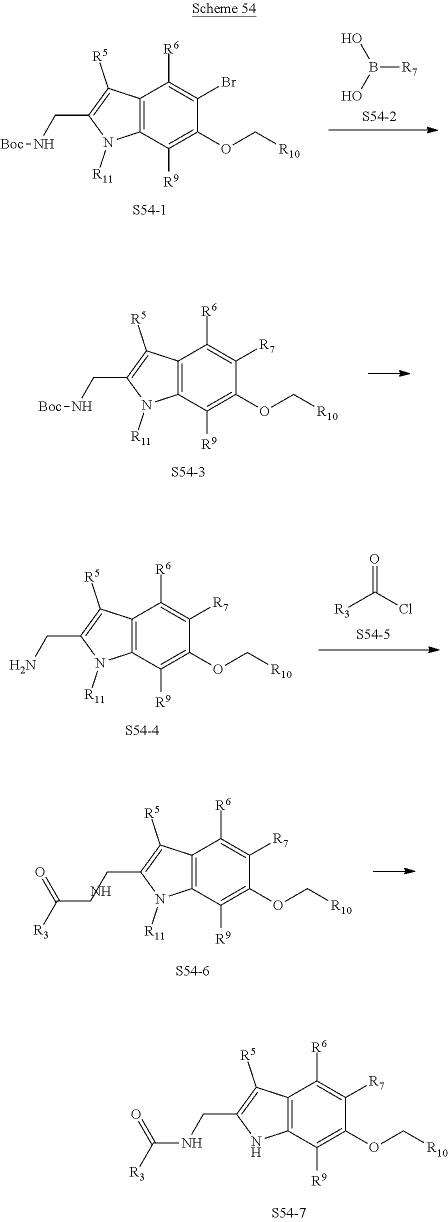

Compounds of formula S54-7 may be prepared according to Scheme 54. Suzuki reaction of compounds such as S54-1 with reagents such as S54-2 in the presence of a catalyst such as cataCXium A Pd G2 and a base such as Cs₂CO₃ in a solvent system such as 2-methylbutan-2-ol and water gives compounds of the formula S54-3. Deprotection of the amine nitrogen using a reagent such as HCl in a solvent such as EtOAc gives compounds of the formula S54-4. Acylation of the amine with a reagent such as S54-5 in the presence of a base such as DIEA in a solvent such as THF gives compounds of the formula S54-6. Deprotection of the indole nitrogen using a reagent such as Mg in a solvent system such as MeOH gives compounds of the formula S54-7.

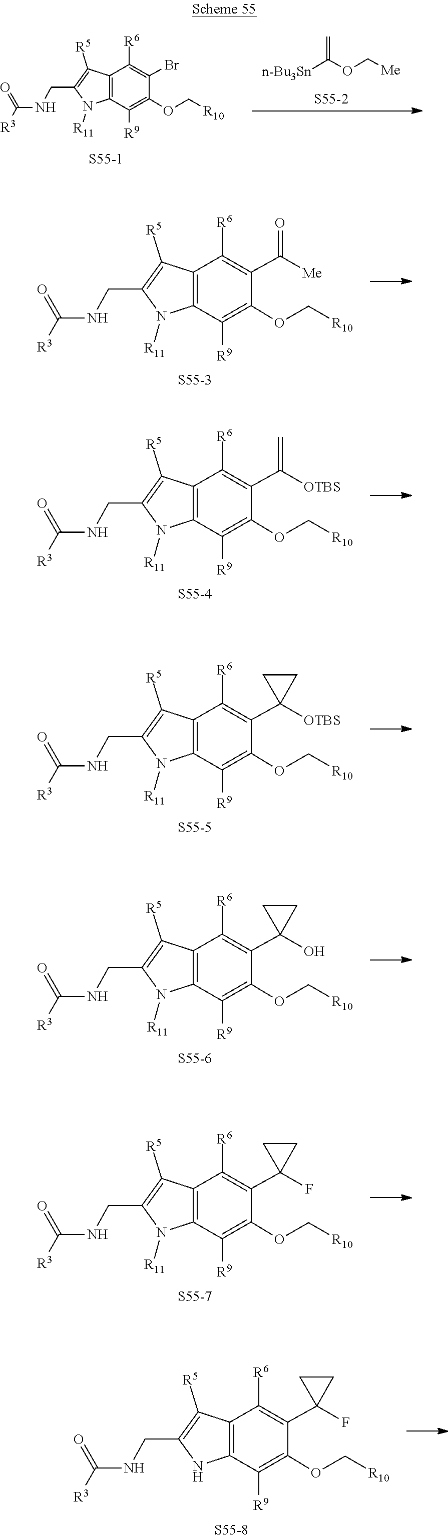

Compounds of formula S55-8 may be prepared according to Scheme 55. Stille reaction of compounds such as S55-1 with reagents such as S55-2 in the presence of a catalyst such as Pd(PPh₃)₂Cl₂ in a solvent such as dioxane gives compounds of the formula S55-3. Formation of the silyl enol ether using a reagent such as TBSOTf and a base such as TEA in a solvent such as DCM gives compounds of the formula S55-4. Cyclopropanation of the olefin with reagents such as ZnEt₂ and CH₂I₂ in a solvent such as DCM gives compounds of the formula S55-5. Deprotection of the silyl ether using a reagent such as TBAF in a solvent such as THF gives compounds of the formula S54-6. Fluorination using a reagent such as DAST in a solvent such as DCM gives compounds of the formula S55-7. Deprotection of the indole nitrogen using reagents such as TBAF in a solvent such as THF gives compounds of the formula S55-8.

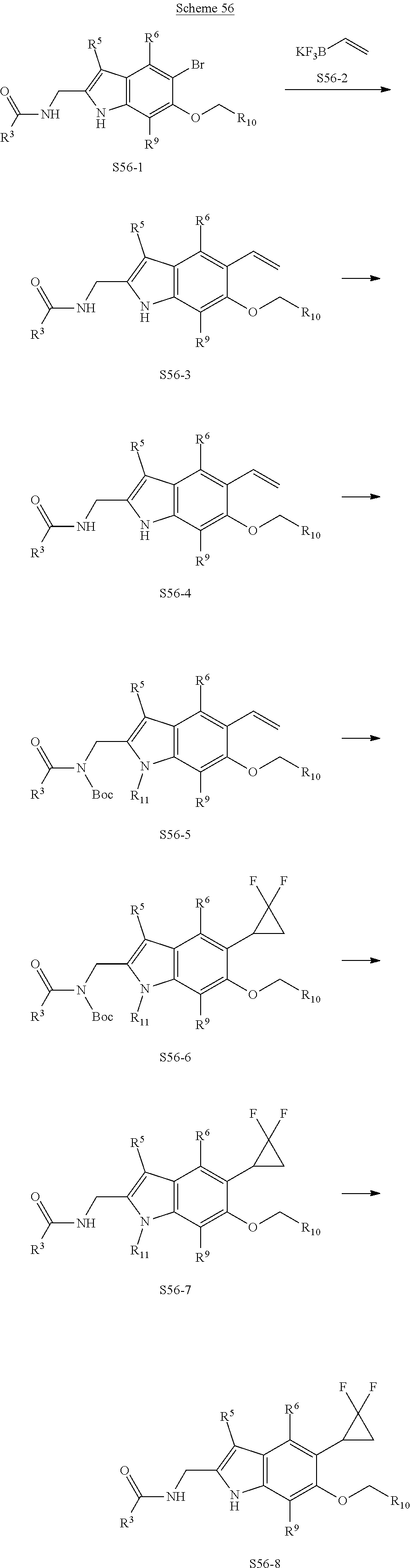

Compounds of formula S56-8 may be prepared according to Scheme 56. Suzuki reaction of compounds such as S56-1 with reagents such as S56-2 in the presence of a catalyst such as Pd(dppf)₂Cl and a base such as TEA in a solvent such as isopropanol gives compounds of the formula S56-3. Protection of the indole nitrogen using a reagent such as benzenesulfonyl chloride and a base such as NaH in a solvent such as DMA gives compounds of the formula S56-4. Protection of the amide nitrogen with a reagent such as Boc₂O and a base such as NaH in a solvent such as DMA gives compounds of the formula S56-5. Cyclopropanation of the olefin using reagents such as TMSBrF₂ and TBAB in a solvent such as toluene gives compounds of the formula S56-6. Deprotection of the amide nitrogen using a reagent such as HCl in a solvent such as ethyl acetate gives compounds of the formula S56-7. Deprotection of the indole nitrogen using reagents such as KOH in a solvent system such as MeOH and water gives compounds of the formula S55-8.

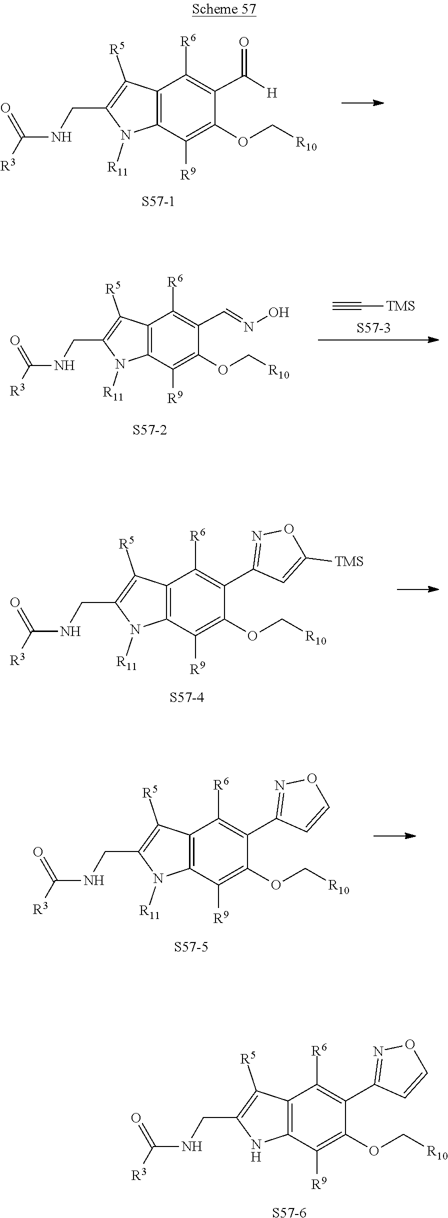

Compounds of formula S57-6 may be prepared according to Scheme 57. Oxime formation using a compound such as S57-1 with reagents such as hydroxylamine hydrochloride and sodium acetate in a solvent system such as ethanol and water gives compounds of the formula S57-2. Heterocycle formation using reagents such as S57-3, NCS, and HCl and a base such as TEA in a solvent such as DMF gives compounds of the formula S57-4. Deprotection of the heterocycle with a reagent such as K₂CO₃ in a solvent such as MeOH gives compounds of the formula S57-5. Deprotection of the indole nitrogen using reagents such as K₂CO₃ in a solvent system such as MeOH and water gives compounds of the formula S57-6.

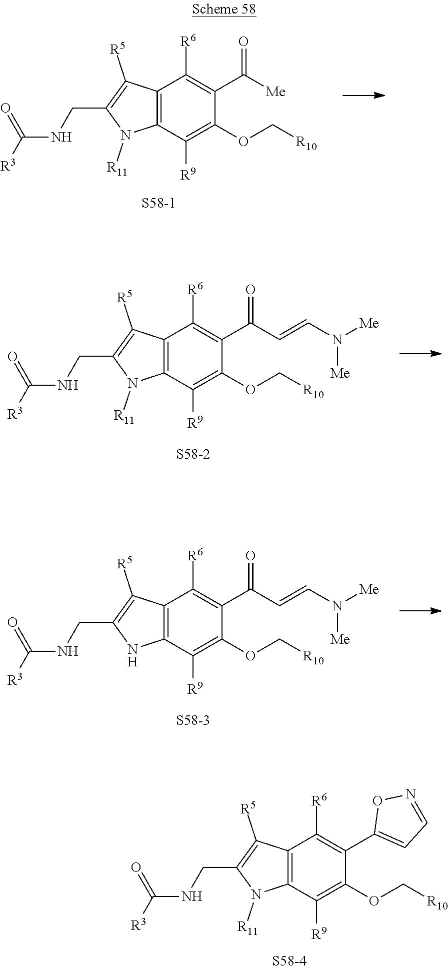

Compounds of formula S58-4 may be prepared according to Scheme 58. Condensation of DMF with a compound such as S58-1 in a solvent such as DMA gives compounds of the formula S58-2. Deprotection of the indole nitrogen using reagents such as TBAF in a solvent such as THF gives compounds of the formula S58-3. Heterocycle formation using reagents such as hydroxylamine hydrochloride in a solvent such as ethanol gives compounds of the formula S58-4.

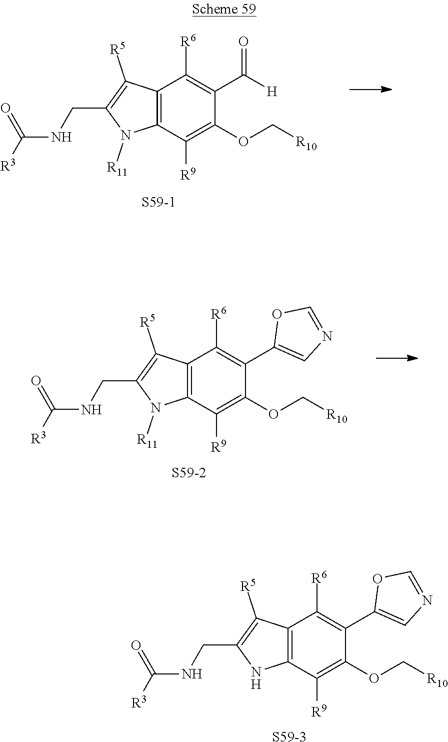

Compounds of formula S59-3 may be prepared according to Scheme 59. Reaction of compounds such as S59-1 with reagents such as TosMIC and a base such as t-BuOK in a solvent such as THF followed by heating in a solvent such as methanol gives compounds of the formula S59-2. Deprotection of the indole nitrogen using reagents such as TBAF in a solvent such as THF gives compounds of the formula S58-3.

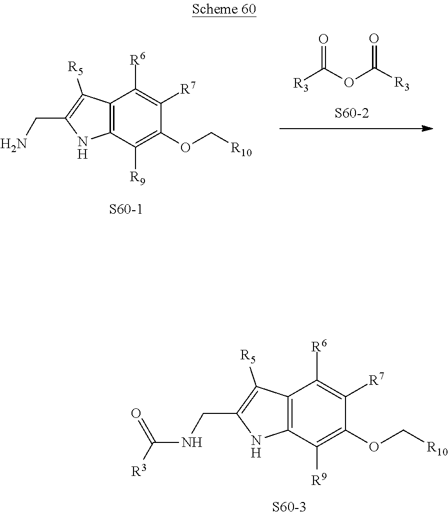

Compounds of formula S60-3 may be prepared according to Scheme 60. Reaction of compounds such as S60-1 with reagents such as S60-2 and a base such as TEA in a solvent such as DCM gives compounds of the formula S59-3.

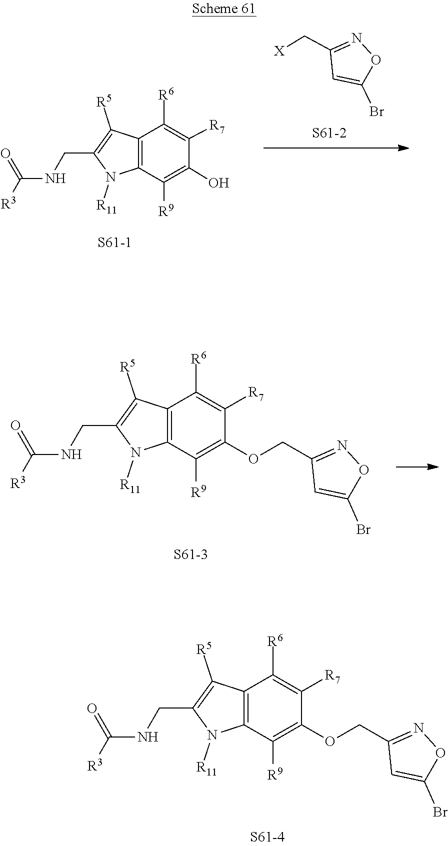

Compounds of formula S61-4 may be prepared according to Scheme 61. Alkylation of compounds such as S61-1 with reagents such as S61-2 and a base such as Cs₂CO₃ in a solvent such as DMF gives compounds of the formula S61-3. Suzuki reaction with a reagent such as (methyl-d₃)boronic acid using a catalyst such as Pd(dppf)Cl₂ and a base such as K₃PO₄ in a solvent system such as dioxane and water gives compounds of the formula S61-4.

Abbreviations used are those conventional in the art and are in accordance with the Periodic Table of the Elements, CAS version, Handbook of Chemistry and Physics, 75^{th} Ed. The following examples are intended to be illustrative only and not limiting in any way.

| | | | |
|---|---|---|---|
| ° C. | degrees Celsius | Cs₂CO₃ | cesium carbonate |
| μL | microliter | d | deuterated (NMR |
| [M + XX]⁺ | observed mass | | solvents) |
| AC₅₀ | half-maximal activity | d | doublet (NMR) |
| | concentration | dd | doublet of doublets |
| ACN | acetonitrile | | (NMR) |
| app | apparent (NMR) | DABCO | 1,4- |
| Boc₂O | Di-tert-butyl dicarbonate | | diazobicyclo[2.2.2]octane |
| BH₃•THF | borane-tetrahydrofuran | DCM | dichloromethane |
| | complex | DIAD | diisopropyl |
| BBr₃ | boron tribromide | | azodicarboxylate |
| calcd | calculated | DMAP | 4-Dimethylaminopyridine |
| Cbz-Cl | benzyl chloroformate | DPPA | Diphenyl phosphoryl |
| CDI | Carbonyldiimidazole | | azide |
| CO₂ | carbon dioxide | DMF | N,N-dimethylformamide |
| EC₅₀ | half-maximal effective | M | molarity or molar |
| | concentration | MS | mass spectrometry |
| EDCI | 1-Ethyl-3-(3- | MsCl | methanesulfonyl chloride |
| | dimethylaminopropyl)car- | Ms₂O | methanesulfonic |
| | bodiimide | | anhydride |
| ESI | electrospray ionization | MTBE | methyl tert-butyl ether |
| EtOAc | ethyl acetate | n/a | not applicable |
| EtOH | ethanol | NBS | N-bromosuccinimide |
| eq | equivalents | NCS | N-chlorosuccinimide |
| g | grams | NIS | N-iodosuccinimide |
| h | hours | NH₄ | ammonium |
| H | hydrogen | NH₄OH | ammonium hydroxide |
| HATU | 1- | NH₄HCO₃ | ammonium bicarbonate |
| | [Bis(dimethylamino)meth- | Na₂SO₄ | sodium sulfate |
| | ylene]-1H-1,2,3- | NaBH₃CN | sodium cyanoborohydride |
| | triazolo[4,5-b]pyridinium | NMI | N-methylimidazole |
| | 3-oxid | NMM | N-methylmorpholine |
| | hexafluorophosphate | NMR | nuclear magnetic |
| HCl | hydrochloric acid | | resonance |
| HOBt | hydroxybenzotriazole | NaOH | sodium hydroxide |
| HPLC | high-performance liquid | PCC | Pyridinium |
| | chromatography | | chlorochromate |
| IC₅₀ | half-maximal inhibitory | PCy₃ | Tricyclohexylphosphine |
| | concentration | Pd₂(dba)₃ | Tris(dibenzylideneacetone) |
| In vacuo | in a vacuum | | dipalladium(0) |
| IUPAC | International Union of | PdCl₂(dppf) | [1,1′- |
| | Pure and Applied | | Bis(diphenylphosphino)fer- |
| | Chemistry | | rocene]dichloropalladi- |
| MHz | megahertz | | um(II) |
| J | J-coupling value (NMR) | pH | potential of hydrogen |
| K₂CO₃ | potassium carbonate | PPh₃ | triphenyl phosphine |
| LDA | lithium diisopropylamide | s | singlet (NMR) |
| LiHMDS | lithium | SFC | super fluid |
| | bis(trimethylsilyl)amide | | chromatography |
| MeOH | Methanol | t | triplet (NMR) |
| MeCN | acetonitrile | T3P | Propanephosphonic acid |
| m | multiplet (NMR) | | anhydride |
| mg | milligrams | TBAB | tetrabutylammonium |
| min | minutes | | bromide |
| mL | milliliter | TBAF | tetrabutylammonium |
| mmol | millimole | | fluoride |
| mM | millimolar | TEA | triethylamine |
| TFA | trifluoroacetic acid | TsCl | p-toluenesulfonyl chloride |
| TCFH | N,N,N′,N′- | wt. % | weight percent |
| | tetramethylchloro- | XPhos | Dicyclohexyl[2′,4′,6′- |
| | formamidinium | | tris(propan-2-y1)[1,1′- |
| | hexafluorophosphate | | biphenyl]-2-yl]phosphane |
| THF | Tetrahydrofuran | Xantphos | (9,9-Dimethyl-9H- |
| TMAD | Tetramethylazodicarbox- | | xanthene-4,5- |
| | amide | | diyl)bis(diphenylphosphane) |
| TMSCl | trimethylsilyl chloride | | |

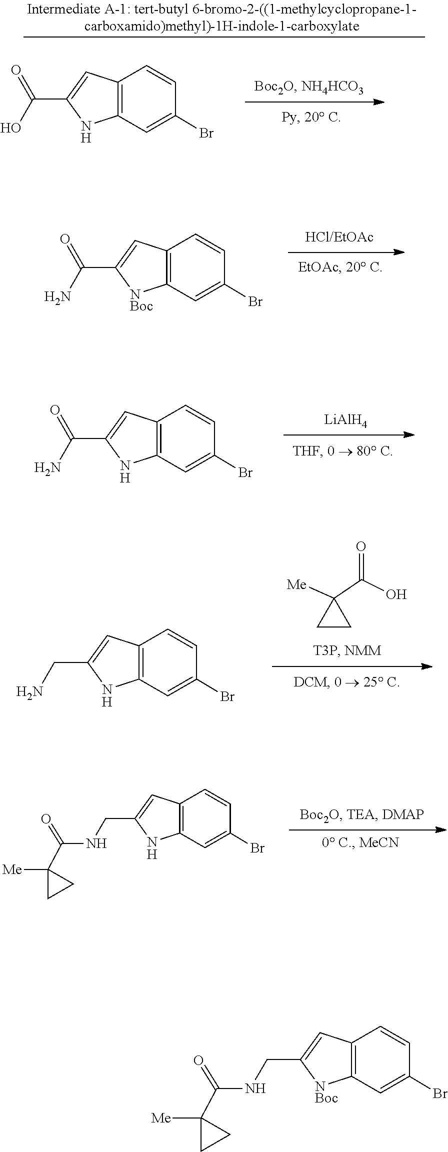

### Step 1: tert-butyl 6-bromo-2-carbamoyl-1H-indole-1-carboxylate

Two parallel reactions were carried out. To a solution of 6-bromo-1H-indole-2-carboxylic acid (7.30 g, 30.4 mmol, 1.00 eq) and pyridine (4.90 mL, 60.8 mmol, 2.00 eq) was added Boc₂O (13.3 g, 60.8 mmol, 13.9 mL, 2.00 eq) followed by NH₄HCO₃ (4.81 g, 60.8 mmol, 2.00 eq). The resulting mixture was stirred at 25° C. for 12 h, after which the two parallel reactions were combined. The resulting mixture was quenched by addition of water (30 mL) at 0° C., and then diluted with water (40 mL) and extracted with ethyl acetate (2×80 mL). The combined organic layers were washed with brine (2×80 mL), dried over anhydrous Na₂SO₄, filtered, and concentrated under reduced pressure. The resulting residue was purified by column chromatography to give tert-butyl 6-bromo-2-carbamoyl-1H-indole-1-carboxylate. LC-MS (ESI) m/z calcd for C₁₄H₁₅BrN₂O₃: 338.0; found 282.9 [M−tBu+H]⁺.

### Step 2: 6-bromo-1H-indole-2-carboxamide

Two parallel reactions were carried out. To a solution of tert-butyl 6-bromo-2-carbamoyl-1H-indole-1-carboxylate (5.00 g, 14.7 mmol, 1.00 eq) in EtOAc (20 mL) was added HCl/EtOAc (4 M, 80 mL, 320 mmol, 21.7 eq), and the resulting mixture was stirred at 25° C. for 12 h. The two parallel reactions were then combined. The mixture was concentrated under reduced pressure to give 6-bromo-1H-indole-2-carboxamide, which was used without further purification. LC-MS (ESI) m/z calcd for C₉H₇BrN₂O: 237.97; found 239.0 [M+H]⁺.

### Step 3: (6-bromo-1H-indol-2-yl)methanamine

To a solution of 6-bromo-1H-indole-2-carboxamide (2.00 g, 8.37 mmol, 1.00 eq) in THF (50 mL) under N₂ at 0° C. was added LiAlH₄ (1.59 g, 41.8 mmol, 5.00 eq) and the resulting mixture was stirred at 0° C. After 0.5 h the reaction was heated to 80° C. and stirred for 1 h. The reaction mixture was quenched by dropwise addition of water (1.6 mL), 15% aqueous NaOH (1.6 mL), and water (3.2 mL). The resulting mixture was filtered then washed with THF (2×20 mL). The filtrate was concentrated under reduced pressure to give (6-bromo-1H-indol-2-yl)methanamine, which was used without further purification. LC-MS (ESI) m/z calcd for C₉H₉BrN₂: 223.99; found 225.1 [M+H]⁺.

### Step 4: N-((6-bromo-1H-indol-2-yl)methyl)-1-methylcyclopropane-1-carboxamide

To a solution of (6-bromo-1H-indol-2-yl)methanamine (1.88 g, 8.35 mmol, 1.00 eq) and 1-methylcyclopropanecarboxylic acid (836 mg, 8.35 mmol, 1.00 eq) in DCM (20 mL) at 0° C. was added NMM (2.75 mL, 25.1 mmol, 3.00 eq) followed by T3P (9.93 mL, 16.7 mmol, 50% purity, 2.00 eq). The resulting mixture was stirred at 25° C. for 0.5 h. The reaction was poured into water (10 mL) and stirred for 2 mins. The aqueous phase was extracted with dichloromethane (3×30 mL). The combined organic phase was washed with brine (2×30 mL), dried over anhydrous Na₂SO₄, filtered, and concentrated under reduced pressure. The resulting residue was purified by column chromatography to give N-((6-bromo-1H-indol-2-yl)methyl)-1-methylcyclopropane-1-carboxamide. LC-MS (ESI) m/z calcd for C₁₄H₁₅BrN₂O: 306.04; found 307.0 [M+H]⁺.

### Step 5: tert-butyl 6-bromo-2-((1-methylcyclopropane-1-carboxamido)methyl)-1H-indole-1-carboxylate

To a solution of N-((6-bromo-1H-indol-2-yl)methyl)-1-methylcyclopropane-1-carboxamide (950 mg, 3.09 mmol, 1.00 eq) in MeCN (10 mL) at 0° C. was added DMAP (3 mg, 309 μmol, 0.10 eq), Boc₂O (1.07 mL, 4.64 mmol, 1.50 eq) and TEA (860 μL, 6.19 mmol, 2.00 eq) and the resulting mixture was stirred at 0° C. for 4 h. The reaction was poured into water (10 mL) and stirred for 5 min. The aqueous phase was extracted with ethyl acetate (2×15 mL). The combined organic phase was washed with brine (2×15 mL), dried over anhydrous Na₂SO₄, filtered, and concentrated under reduced pressure. The crude residue was purified by column chromatography to give tert-butyl 6-bromo-2-((1-methylcyclopropane-1-carboxamido)methyl)-1H-indole-1-carboxylate. LC-MS (ESI) m/z calcd for C₁₉H₂₃BrN₂O₃: 406.09; found 407.0 [M+H]⁺.

The following compounds in Table B-1 were synthesized using procedures similar to Intermediate A-1 using the appropriate starting materials.

TABLE B-1

| Inter- | | | | LCMS, |
|---|---|---|---|---|
| mediate | | | Exact mass | found |
| No. | Structure | IUPAC name | (g/mol) | [M + H]⁺ |
| B-1-1 | 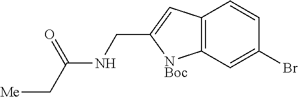 [SMILES: CCC(=O)NCc1cc2ccc(Br)cc2n1C(=O)OC(C)(C)C] | tert-butyl 6-bromo-2- (propionamidomethyl)- 1H-indole-1- carboxylate | 380.1 | 381.0 |

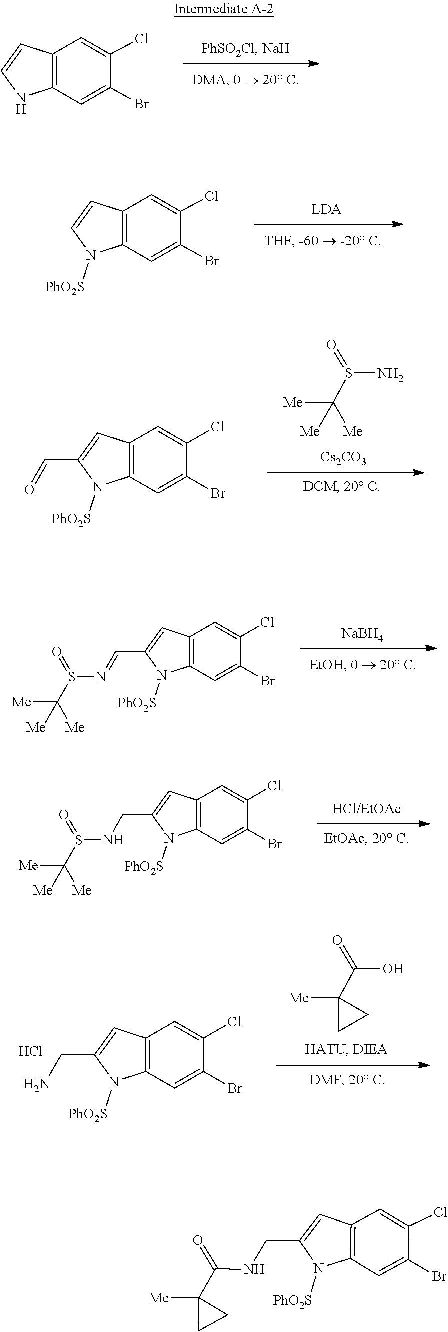

### Step 1: 6-bromo-5-chloro-1-(phenylsulfonyl)-1H-indole

To a solution of NaH (781 mg, 19.5 mmol, 60% in mineral oil, 1.50 eq) in DMA (20 mL) was added 6-bromo-5-chloro-1H-indole (3.00 g, 13.0 mmol, 1.00 eq) in DMA (10 mL) at 0° C. under N₂. The mixture was stirred at 0° C. for 0.5 h, whereupon benzenesulfonyl chloride (3.45 g, 19.5 mmol, 1.50 eq) was added. The mixture was allowed to warm to 20° C. and stir for 1.5 h. The reaction mixture was poured into water (40 mL) and stirred for 3 mins. The aqueous phase was extracted with ethyl acetate (3×40 mL). The combined organic phases were dried over anhydrous Na₂SO₄, filtered, and concentrated in vacuo. The residue was purified by silica gel chromatography to give 6-bromo-5-chloro-1-(phenylsulfonyl)-1H-indole. LC-MS (ESI) m/z calcd for C₁₄H₉BrClNO₂S: 368.92; found: 370.0 [M+H]⁺.

### Step 2: 6-bromo-5-chloro-1-(phenylsulfonyl)-1H-indole-2-carbaldehyde

Two reactions were carried out in parallel as described. To a solution of 6-bromo-5-chloro-1-(phenylsulfonyl)-1H-indole (3.45 g, 9.31 mmol, 1.00 eq) in THF (30 mL) was added LDA (2 M in THF, 6.98 mL, 14.0 mmol, 1.50 eq) at −60° C., and the mixture was stirred at −60° C. for 0.5 h. DMF (1.07 mL, 13.9 mmol, 1.50 eq) was then added. The reaction mixture was allowed to warm to 20° C. and stirred for 1 h. The two reactions were then combined for work up. The combined reaction mixture was poured into water (50 mL) and stirred for 2 min. The aqueous phase was extracted with ethyl acetate (3×50 mL). The combined organic phases were dried over anhydrous Na₂SO₄, filtered and concentrated in vacuo. The crude product was triturated with MTBE (50 mL) at 20° C. for 30 min to give 6-bromo-5-chloro-1-(phenylsulfonyl)-1H-indole-2-carbaldehyde. LC-MS (ESI) m/z calcd for C₁₅H₉BrClNO₃S: 396.92; found: 397.9 [M+H]⁺.

### Step 3: (E)-N-((6-bromo-5-chloro-1-(phenylsulfonyl)-1H-indol-2-yl)methylene)-2-methylpropane-2-sulfinamide

Two reactions were carried out in parallel as described. To a solution of 6-bromo-5-chloro-1-(phenylsulfonyl)-1H-indole-2-carbaldehyde (2.90 g, 7.27 mmol, 1.00 eq) in DCM (30 mL) were added 2-methylpropane-2-sulfinamide (1.76 g, 14.5 mmol, 2.00 eq) and Cs₂CO₃ (4.74 g, 14.5 mmol, 2.00 eq). The mixture was stirred at 20° C. for 2 h. The two reactions were then combined, and the combined reaction mixture was filtered and concentrated in vacuo. The residue was purified by silica gel chromatography to give (E)-N-((6-bromo-5-chloro-1-(phenylsulfonyl)-1H-indol-2-yl)methylene)-2-methylpropane-2-sulfinamide. LC-MS (ESI) m/z calcd for C₁₉H₁₈BrClN₂O₃S₂: 499.96; found: 501.0 [M+H]⁺.

### Step 4: N-((6-bromo-5-chloro-1-(phenylsulfonyl)-1H-indol-2-yl)methyl)-2-methylpropane-2-sulfinamide

Two reactions were carried out in parallel as described. To a solution of (E)-N-((6-bromo-5-chloro-1-(phenylsulfonyl)-1H-indol-2-yl)methylene)-2-methylpropane-2-sulfinamide (2.90, 5.78 mmol, 1.00 eq) in EtOH (30 mL) was added NaBH₄ (437 mg, 11.5 mmol, 2.00 eq) at 0° C. under N₂. The mixture was allowed to warm to 20° C. and stir for 1 h. The two reactions were combined for work up, and the combined reaction mixture was poured into water (30 mL) and stirred for 2 min. The aqueous phase was extracted with ethyl acetate (3×30 mL). The combined organic phase was washed with brine (30 mL), dried over anhydrous Na₂SO₄, filtered and concentrated in vacuum to give N-((6-bromo-5-chloro-1-(phenylsulfonyl)-1H-indol-2-yl)methyl)-2-methylpropane-2-sulfinamide, which was used without further purification. LC-MS (ESI) m/z calcd for C₁₉H₂₀BrClN₂O₃S₂: 501.98; found: 503.0 [M+H]⁺.

### Step 5: (6-bromo-5-chloro-1-(phenylsulfonyl)-1H-indol-2-yl)methanamine hydrochloride

To a solution of N-((6-bromo-5-chloro-1-(phenylsulfonyl)-1H-indol-2-yl)methyl)-2-methylpropane-2-sulfinamide (3.50 g, 6.95 mmol, 1.00 eq) in EtOAc (30 mL) was added HCl (4 M in EtOAc, 30 mL, 120 mmol, 17.3 eq). The mixture was stirred at 20° C. for 1 h. The reaction mixture was filtered and the filter cake was washed with EtOAc (20 mL), dried under vacuum to give (6-bromo-5-chloro-1-(phenylsulfonyl)-1H-indol-2-yl)methanamine hydrochloride, which was used without further purification. LC-MS (ESI) m/z calcd for C₁₅H₁₂BrClN₂O₂S: 397.95; found: 398.9 [M+H]⁺.

### Step 6: N-((6-bromo-5-chloro-1-(phenylsulfonyl)-1H-indol-2-yl)methyl)-1-methylcyclopropanecarboxamide

To a solution of 1-methylcyclopropanecarboxylic acid (689 mg, 6.88 mmol, 1.20 eq) in DMF (20 mL) was added HATU (3.27 g, 8.60 mmol, 1.50 eq), and the mixture was stirred at 20° C. for 0.5 h, whereupon DIEA (4 mL, 22.9 mmol, 4.00 eq) and (6-bromo-5-chloro-1-(phenylsulfonyl)-1H-indol-2-yl)methanamine hydrochloride (2.50 g, 5.73 mmol, 1.00 eq) were added. The resulting mixture was stirred at 20° C. for 1 h. The reaction mixture was poured into water (40 mL) and stirred for 2 min. The aqueous phase was extracted with ethyl acetate (3×40 mL). The combined organic phases were washed with brine, dried over anhydrous Na₂SO₄, filtered, and concentrated under reduced pressure. Purification by silica gel chromatography gave N-((6-bromo-5-chloro-1-(phenylsulfonyl)-1H-indol-2-yl)methyl)-1-methylcyclopropanecarboxamide, Intermediate A-2. LC-MS (ESI) m/z calcd for C₂₀H₁₈BrClN₂O₃S: 479.99; found: 481.0 [M+H]⁺.

The following compounds in Table B-2 were synthesized using procedures similar to Intermediate A-2 using the appropriate starting materials.

TABLE B-2

| | | | | LCMS, |
|---|---|---|---|---|
| Intermediate | | | Exact mass | found |
| No. | Structure | IUPAC name | (g/mol) | [M + H]⁺ |
| B-2-1 | 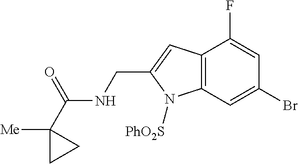 | N-((6-bromo-4- fluoro-1- (phenylsulfonyl)- 1H-indol-2- yl)methyl)-1- methylcyclopropane- 1-carboxamide | 464.02 | 465.1 |
| B-2-2 | 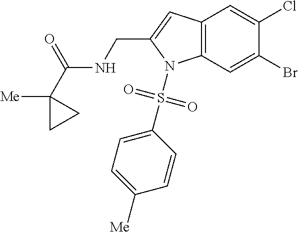 [SMILES: Cc1ccc(S(=O)(=O)n2c(CNC(=O)C3(C)CC3)cc3cc(Cl)c(Br)cc32)cc1] | N-((6-bromo-5- chloro-1-tosyl-1H- indol-2-yl)methyl)- 1- methylcyclopropane- 1-carboxamide | 494.01 | 495.0 |

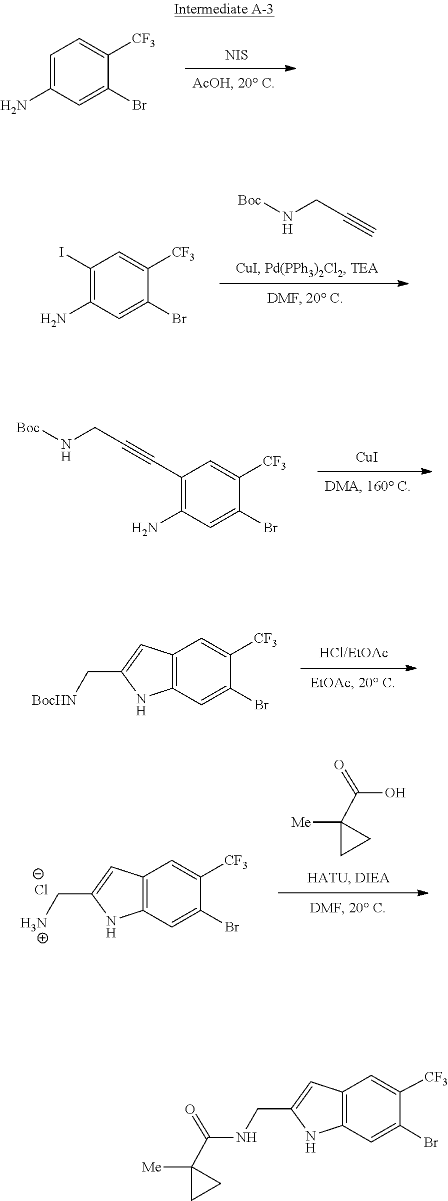

### Step 1: 5-bromo-2-iodo-4-(trifluoromethyl)aniline

Two reactions were carried out in parallel as described. To a solution of 3-bromo-4-(trifluoromethyl)aniline (50.0 g, 208 mmol, 1.00 eq) in AcOH (500 mL) was added NIS (46.9 g, 208 mmol, 1.00 eq), and the mixture was stirred for 2 h. The two reactions were then combined for work up. The combined reaction mixture was poured into water (500 mL) and stirred for 5 min. The mixture was adjusted to pH 7 by addition of saturated aqueous Na₂CO₃, the aqueous phase was extracted with ethyl acetate (500 mL×2). The combined organic phase was washed with brine (500 mL×2), dried over anhydrous Na₂SO₄, filtered and concentrated in vacuo. Purification by silica gel chromatography gave 5-bromo-2-iodo-4-(trifluoromethyl)aniline. LC-MS (ESI) m/z calcd for C₇H₄BrF₃IN: 364.85; found 365.8 [M+H]⁺.

### Step 2: tert-butyl (3-(2-amino-4-bromo-5-(trifluoromethyl)phenyl)prop-2-yn-1-yl)carbamate

Three reactions were carried out in parallel as described. To a solution of 5-bromo-2-iodo-4-(trifluoromethyl)aniline (47.0 g, 128 mmol, 1.00 eq) and tert-butyl prop-2-yn-1-ylcarbamate (23.9 g, 154 mmol, 1.20 eq) in DMF (400 mL) were added TEA (90.0 mL, 642 mmol, 5.00 eq), CuI (4.90 g, 25.7 mmol, 0.20 eq), and Pd(PPh₃)₂Cl₂ (9.00 g, 12.8 mmol, 0.10 eq) under N₂, and the resulting mixture was stirred for 16 h. Three reactions were then combined for work up. The combined reaction mixture was poured into ice-water (500 mL) and stirred for 5 min. The aqueous phase was extracted with ethyl acetate (3×500 mL). The combined organic phase was washed with brine (2×700 mL), dried with anhydrous Na₂SO₄, filtered and concentrated in vacuo. Purification by silica gel chromatography gave tert-butyl (3-(2-amino-4-bromo-5-(trifluoromethyl)phenyl)prop-2-yn-1-yl)carbamate. LC-MS (ESI) m/z calcd for C₁₅H₁₆BrF₃N₂O₂: 392.03; found 393.1 [M+H]⁺.

### Step 3: tert-butyl ((6-bromo-5-(trifluoromethyl)-1H-indol-2-yl)methyl)carbamate

Three reactions were carried out in parallel as described. To a solution of tert-butyl (3-(2-amino-4-bromo-5-(trifluoromethyl)phenyl)prop-2-yn-1-yl)carbamate (23.0 g, 58.5 mmol, 1.00 eq) in DMA (200 mL) was added CuI (5.60 g, 29.3 mmol, 0.50 eq), and the resulting mixture was heated to 160° C. and stirred for 1.5 h. After allowing the reaction mixtures to cool to room temperature, the three reactions were combined for work up. The combined mixture was poured into water (150 mL) and stirred for 5 min. The aqueous phase was extracted with ethyl acetate (3×150 mL). The combined organic phase was washed with brine (2×150 mL), dried over anhydrous Na₂SO₄, filtered, and concentrated under reduced pressure. Purification by silica gel chromatography gave tert-butyl ((6-bromo-5-(trifluoromethyl)-1H-indol-2-yl)methyl)carbamate. LC-MS (ESI) m/z calcd for C₁₅H₁₆BrF₃N₂O₂: 392.03; found 337.0 [M−tBu+H]⁺.

### Step 4: (6-bromo-5-(trifluoromethyl)-1H-indol-2-yl)methanamine hydrochloride

Two reactions were carried out in parallel as described. To a solution of tert-butyl ((6-bromo-5-(trifluoromethyl)-1H-indol-2-yl)methyl)carbamate (17.0 g, 43.2 mmol, 1.00 eq) in EtOAc (30 mL) was added 4M HCl in EtOAc (245 mL, 980 mmol, 22.6 eq), and the mixture was stirred at room temperature for 2 h. The two reactions were then combined for work up. The combined reaction mixture was concentrated under reduced pressure to give (6-bromo-5-(trifluoromethyl)-1H-indol-2-yl)methanamine hydrochloride, which was used without further purification. LC-MS (ESI) m/z: [M+H]⁺ calcd for C₁₀H₈BrF₃N₂: 291.98; found 276.0 [M−NH₂]⁺.

### Step 5: N-((6-bromo-5-(trifluoromethyl)-1H-indol-2-yl)methyl)-1-methylcyclopropanecarboxamide

Two reactions were carried out in parallel as described. To a solution of 1-methylcyclopropanecarboxylic acid (4.56 g, 45.5 mmol, 1.00 eq) in DMF (100 mL) was added HATU (26.0 g, 68.3 mmol, 1.50 eq), and the mixture was stirred for 10 min. The reaction mixture was then cooled to 0° C., and 6-Bromo-5-(trifluoromethyl)-1H-indol-2-yl)methanamine hydrochloride (15.0 g, 45.5 mmol, 1.00 eq) and DIEA (32 mL, 182 mmol, 4.00 eq) were added. The resulting mixture was allowed to warm to room temperature and stir for 1 h. The two reactions were then combined for work up. The combined mixture was poured into water (100 mL) and stirred for 5 min. The aqueous phase was extracted with ethyl acetate (2×200 mL). The combined organic phase was washed with brine (2×300 mL), dried over anhydrous Na₂SO₄, filtered, and concentrated in vacuo. Purification by silica gel chromatography gave N-((6-bromo-5-(trifluoromethyl)-1H-indol-2-yl)methyl)-1-methylcyclopropanecarboxamide (Intermediate A-3). LC-MS (ESI) m/z calcd for C₁₅H₁₄BrF₃N₂O: 374.02; found 375.1 [M+H]⁺.

The following compounds in Table B-3 were synthesized using procedures similar to Intermediate A-3 using the appropriate starting materials.

TABLE B-3

| | | | Exact | LCMS, |
|---|---|---|---|---|
| Intermediate | | | mass | found |
| No. | Structure | IUPAC name | (g/mol) | [M + H]⁺ |
| B-3-1 | 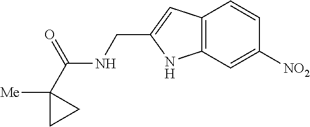 [SMILES: CC1(C(=O)NCc2cc3ccc([N+](=O)[O-])cc3[nH]2)CC1] | 1-methyl-N-((6-nitro- 1H-indol-2- yl)methyl)cyclopropane- 1-carboxamide | 273.11 | 274.2 |
| B-3-2 | 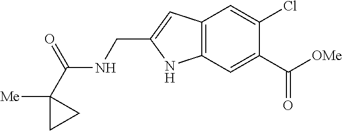 [SMILES: COC(=O)c1cc2[nH]c(CNC(=O)C3(C)CC3)cc2cc1Cl] | methyl 5-chloro-2-((1- methylcyclopropane-1- carboxamido)methyl)- 1H-indole-6-carboxylate | 320.09 | 321.1 |
| B-3-3 | 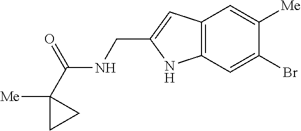 [SMILES: Cc1cc2cc(CNC(=O)C3(C)CC3)[nH]c2cc1Br] | N-((6-bromo-5-methyl- 1H-indol-2-yl)methyl)- 1-methylcyclopropane- 1-carboxamide | 320.05 | 321.1 |
| B-3-4 | 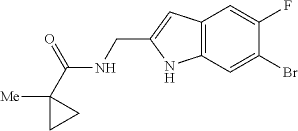 [SMILES: CC1(C(=O)NCc2cc3cc(F)c(Br)cc3[nH]2)CC1] | N-((6-bromo-5-fluoro- 1H-indol-2-yl)methyl)- 1-methylcyclopropane- 1-carboxamide | 324.03 | 325.0 |
| B-3-5 | 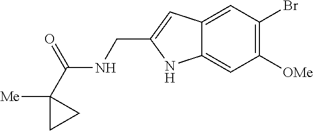 [SMILES: COc1cc2[nH]c(CNC(=O)C3(C)CC3)cc2cc1Br] | N-((5-bromo-6- methoxy-1H-indol-2- yl)methyl)-1- methylcyclopropane-1- carboxamide | 336.05 | 337.0 |

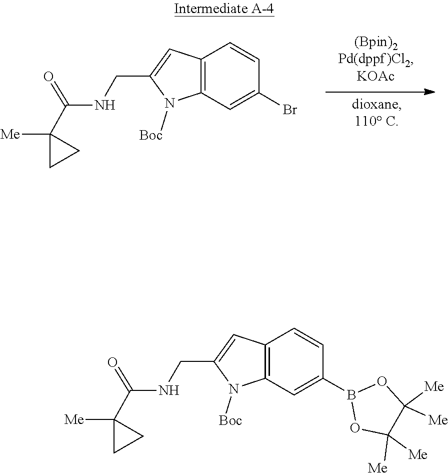 [SMILES: CC(C)(C)OC(=O)n1c(CNC(=O)C2(C)CC2)cc2ccc(Br)cc21 ; CC(C)(C)OC(=O)n1c(CNC(=O)C2(C)CC2)cc2ccc(B3OC(C)(C)C(C)(C)O3)cc21]

### Step 1: tert-butyl 2-((1-methylcyclopropanecarboxamido)methyl)-6-(4,4,5,5-tetramethyl-1,3,2-dioxaborolan-2-yl)-1H-indole-1-carboxylate

To a solution of tert-butyl 6-bromo-2-((1-methylcyclopropanecarboxamido)methyl)-1H-indole-1-carboxylate (1.00 g, 2.46 mmol, 1.00 eq) and 4,4,5,5-tetramethyl-2-(4,4,5,5-tetramethyl-1,3,2-dioxaborolan-2-yl)-1,3,2-dioxaborolane (748 mg, 2.95 mmol, 1.20 eq) in dioxane (10 mL) were added KOAc (723 mg, 7.37 mmol, 3.00 eq) and Pd(dppf)Cl₂ (359 mg, 491 μmol, 0.20 eq) under N₂, and the mixture was stirred at 110° C. for 1 h. The mixture was allowed to cool to room temperature before it was filtered, poured into water (15 mL) and stirred for 5 min. The aqueous phase was extracted with ethyl acetate (2×10 mL). The combined organic phases were washed with brine (2×10 mL), dried over anhydrous Na₂SO₄, filtered and concentrated under reduced pressure. The residue was purified by silica gel chromatography to give tert-butyl 2-((1-methylcyclopropanecarboxamido)methyl)-6-(4,4,5,5-tetramethyl-1,3,2-dioxaborolan-2-yl)-1H-indole-1-carboxylate, Intermediate A-4. LC-MS (ESI) m/z calcd for C₂₅H₃₅BN₂O₅: 454.26; found: 455.3 [M+H]⁺.

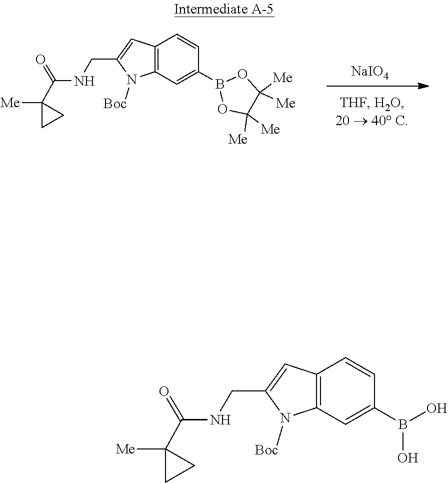 [SMILES: CC(C)(C)OC(=O)n1c(CNC(=O)C2(C)CC2)cc2ccc(B3OC(C)(C)C(C)(C)O3)cc21 ; "NaIO4" ; CC(C)(C)OC(=O)n1c(CNC(=O)C2(C)CC2)cc2ccc(B(O)O)cc21]

### Step 1: (1-(tert-butoxycarbonyl)-2-((1-methylcyclopropanecarboxamido)methyl)-1H-indol-6-yl)boronic acid

To a mixture of tert-butyl 2-((1-methylcyclopropanecarboxamido)methyl)-6-(4,4,5,5-tetramethyl-1,3,2-dioxaborolan-2-yl)-1H-indole-1-carboxylate (1.10 g, 2.42 mmol, 1.00 eq) in THF (9 mL) and H₂O (1 mL) was added NaIO₄ (2.07 g, 9.68 mmol, 4.00 eq) in one portion at 20° C. The mixture was then stirred at 40° C. for 16 h. The mixture was allowed to cool to room temperature, then was filtered and concentrated in vacuo to give (1-(tert-butoxycarbonyl)-2-((1-methylcyclopropanecarboxamido)methyl)-1H-indol-6-yl)boronic acid, Intermediate A-5. LC-MS (ESI) m/z calcd for C₁₉H₂₅BN₂O₅: 372.19; found 373.2 [M+H]⁺.

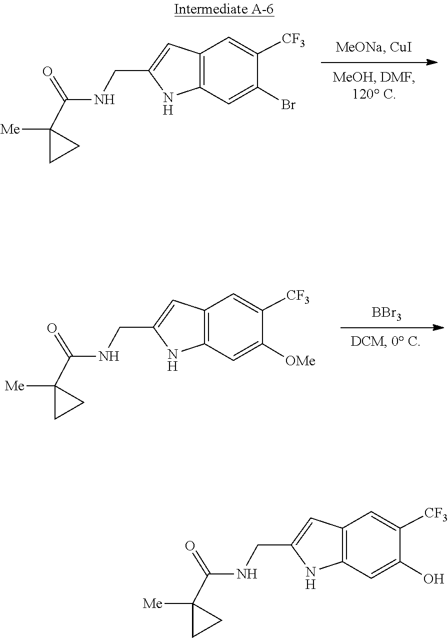 [SMILES: CC1(C(=O)NCc2cc3cc(C(F)(F)F)c(Br)cc3[nH]2)CC1 ; COc1cc2[nH]c(CNC(=O)C3(C)CC3)cc2cc1C(F)(F)F ; "BBr3" ; CC1(C(=O)NCc2cc3cc(C(F)(F)F)c(O)cc3[nH]2)CC1]

### Step 1: N-((6-methoxy-5-(trifluoromethyl)-1H-indol-2-yl)methyl)-1-methylcyclopropane-1-carboxamide

Three reactions were carried out in parallel as described. To a mixture of N-((6-bromo-5-(trifluoromethyl)-1H-indol-2-yl)methyl)-1-methylcyclopropane-1-carboxamide (9.10 g, 24.3 mmol, 1.00 eq) and CH₃ONa (88.0 g, 485 mmol, 30% in MeOH, 20.0 eq) in DMF (85 mL) and MeOH (85 mL) was added CuI (11.6 g, 60.6 mmol, 2.50 eq) at 20° C. under N₂. The mixture then heated to 120° C. and allowed to stir for 2 h. After cooling to room temperature, the three reactions were combined for work up, poured into water (100 mL), and stirred for 5 min. The mixture was filtered and the aqueous phase was extracted with ethyl acetate (3×300 mL). The combined organic phases were washed with brine (2×300 mL), dried over anhydrous Na₂SO₄, filtered, and concentrated under reduced pressure. The residue was purified by silica gel chromatography to give N-((6-methoxy-5-(trifluoromethyl)-1H-indol-2-yl)methyl)-1-methylcyclopropane-1-carboxamide. LC-MS (ESI) m/z calcd for C₁₆H₁₇F₃N₂O₂: 326.12; found: 327.2 [M+H]⁺.

### Step 2: N-((6-hydroxy-5-(trifluoromethyl)-1H-indol-2-yl)methyl)-1-methylcyclopropane-1-carboxamide

Four reactions were carried out in parallel as described. To a mixture of N-((6-methoxy-5-(trifluoromethyl)-1H-indol-2-yl)methyl)-1-methylcyclopropane-1-carboxamide (5.00 g, 15.3 mmol, 1.00 eq) in DCM (70 mL) was added BBr₃ (61.3 mmol, 5.95 mL, 4.00 eq) dropwise at 0° C. under N₂, and the mixture was stirred at 0° C. for 2 h. Four reactions were combined for work up. The mixture was poured into water (100 mL) at 0° C., adjusted to pH 7 by addition of saturated aqueous NaHCO₃, then extracted with ethyl acetate (3×100 mL). The combined organic phases were washed with brine (200 mL), dried over Na₂SO₄, filtered and concentrated under reduced pressure. The residue was purified by silica gel chromatography to give N-((6-hydroxy-5-(trifluoromethyl)-1H-indol-2-yl)methyl)-1-methylcyclopropane-1-carboxamide, Intermediate A-6. LC-MS (ESI) m/z calcd for C₁₅H₁₅F₃N₂O₂: 312.11; found: 313.1 [M+H]⁺.

The following compounds in Table B-6 were synthesized using procedures similar to Intermediate A-6 using the appropriate starting materials.

TABLE B-6

| | | | Exact | LCMS, |
|---|---|---|---|---|
| Intermediate | | | mass | found |
| No. | Structure | IUPAC name | (g/mol) | [M + H]⁺ |
| B-6-1 | 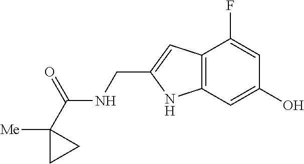 [SMILES: CC1(C(=O)NCc2cc3c(F)cc(O)cc3[nH]2)CC1] | N-((4-fluoro-6- hydroxy-1H-indol-2- yl)methyl)-1- methylcyclopropane- 1-carboxamide | 262.11 | 263.2 |

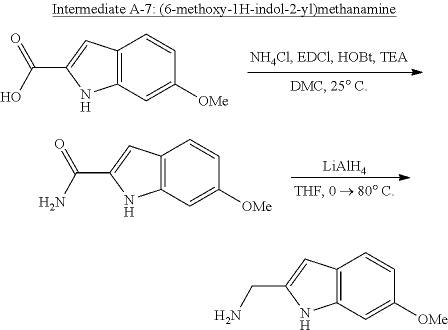

### Step 1: 6-methoxy-1H-indole-2-carboxamide

To a mixture of 6-methoxy-1H-indole-2-carboxylic acid (10 g, 52.3 mmol, 1.00 eq) in DCM (100 mL) was added 1-(3-Dimethylaminopropyl)-3-ethylcarbodiimide hydrochloride (EDCI, 12.0 g, 62.7 mmol, 1.20 eq) and 1-Hydroxybenzotriazole (8.48 g, 62.7 mmol, 1.20 eq) in one portion at 25° C. under N₂. The mixture was stirred at 25° C. for 10 min. TEA (78.5 mmol, 10.9 mL, 1.50 eq) and NH₄Cl (4.20 g, 78.4 mmol, 1.50 eq) were then added, and the mixture was stirred at 25° C. for 2 h. The reaction mixture was poured into water (50 mL) and stirred for 5 min. The aqueous phase was extracted with ethyl acetate (2×50 mL). The combined organic phases were washed with brine (2×50 mL), dried over anhydrous Na₂SO₄, filtered, and concentrated under reduced pressure to give a residue. The residue was purified by silica gel chromatography to give 6-methoxy-1H-indole-2-carboxamide. LC-MS (ESI) m/z calcd for C₁₀H₁₀N₂O₂: 190.07; found 191.1 [M+H]⁺.

### Step 2: (6-methoxy-1H-indol-2-yl)methanamine

To a mixture of 6-methoxy-1H-indole-2-carboxamide (3.00 g, 15.7 mmol, 1.00 eq) in THF (60 mL) was carefully added LiAlH₄ (4.19 g, 110 mmol, 7.00 eq) portion-wise at 0° C. under N₂. After the addition, the reaction mixture was allowed to warm to 25° C. and stirred for 30 min. Then the mixture was heated to 80° C. and allowed to stir for 16 h. The reaction mixture was cooled to 0° C., then was quenched by addition of water (4.2 mL), diluted with water (12 mL) and aq. NaOH (4.2 mL, 15% w/w), filtered and extracted with ethyl acetate (2×30 mL). The combined organic layers were washed with brine (2×30 mL), dried over anhydrous Na₂SO₄, filtered, and concentrated under reduced pressure to give (6-methoxy-1H-indol-2-yl)methanamine (Intermediate A-7) which was used further purification. LC-MS (ESI) m/z calcd for C₁₀H₁₂N₂O: 176.09; found 177.1 [M+H]⁺.

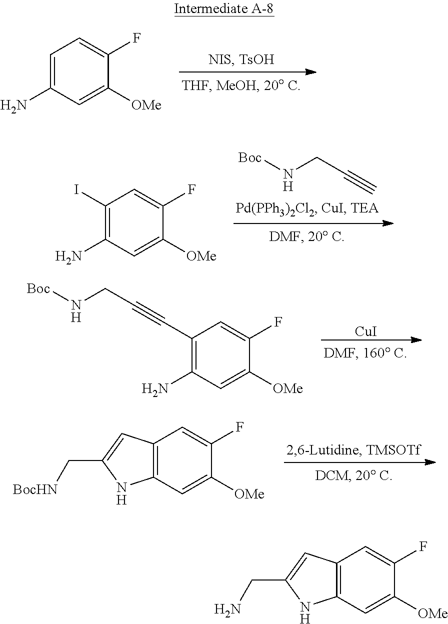

### Step 1: 4-fluoro-2-iodo-5-methoxyaniline

To a solution of 4-fluoro-3-methoxyaniline (30.0 g, 212 mmol, 1.00 eq) in THF (250 mL) and MeOH (250 mL) were added N-iodosuccinimide (38.2 g, 170 mmol, 0.80 eq) and 4-methylbenzenesulfonic acid (36.6 g, 212 mmol, 1.00 eq), and the mixture was stirred at 20° C. for 3 h. To the mixture was added water (700 mL) followed by extraction with ethyl acetate (2×600 mL). The combined organic phases were washed with brine (500 mL), dried over anhydrous sodium sulfate, filtered, and concentrated under reduced pressure. The residue was purified by silica gel chromatography to give 4-fluoro-2-iodo-5-methoxyaniline, which was used without further purification. LC-MS (ESI) m/z calcd for C₇H₇FlNO: 266.96; found: 268.0 [M+H]⁺.

### Step 2: tert-butyl (3-(2-amino-5-fluoro-4-methoxyphenyl) prop-2-yn-1-yl) carbamate

To a solution of 4-fluoro-2-iodo-5-methoxyaniline (33.0 g, 123 mmol, 1.00 eq) and tert-butyl prop-2-yn-1-ylcarbamate (21.1 g, 135 mmol, 1.10 eq) in DMF (160 mL) were added CuI (2.35 g, 12.3 mmol, 0.10 eq), TEA (86 mL, 617 mmol, 5.00 eq) and Pd(PPh₃)₂Cl₂ (4.34 g, 6.18 mmol, 0.05 eq) under N₂. The reaction mixture was stirred at 20° C. for 6 h under N₂. The reaction mixture was quenched by addition of water (500 mL) and extracted with ethyl acetate (3×300 mL). The combined organic layers were washed with brine (500 mL), dried over anhydrous sodium sulfate, filtered and concentrated under reduced pressure. The residue was purified by silica gel chromatography to give tert-butyl (3-(2-amino-5-fluoro-4-methoxyphenyl) prop-2-yn-1-yl) carbamate, which was used without further purification. LC-MS (ESI) m/z calcd for C₁₅H₁₉FN₂O₃: 294.14; found: 295.3 [M+H]⁺.

### Step 3: tert-butyl ((5-fluoro-6-methoxy-1H-indol-2-yl) methyl) carbamate

To a solution of tert-butyl (3-(2-amino-5-fluoro-4-methoxyphenyl) prop-2-yn-1-yl) carbamate (10.0 g, 33.9 mmol, 1.00 eq) in DMF (100 mL) was added CuI (3.24 g, 16.9 mmol, 0.50 eq) under N₂. The reaction mixture was heated to 160° C. and stirred for 0.5 h under N₂. After allowing the mixture to cool to room temperature, the mixture was poured into water (200 mL) and stirred for 5 min. The resulting suspension was filtered, the filter cake was washed with water (100 mL) to remove DMF, then washed with ethyl acetate (100 mL), dried over anhydrous sodium sulfate, filtered, and concentrated under reduced pressure. The residue was purified by silica gel chromatography to give tert-butyl ((5-fluoro-6-methoxy-1H-indol-2-yl) methyl) carbamate. LC-MS (ESI) m/z calcd for C₁₅H₁₉FN₂O₃: 294.14; found: 295.1 [M+H]⁺.

### Step 4: (5-fluoro-6-methoxy-1H-indol-2-yl) methanamine

To a solution of tert-butyl ((5-fluoro-6-methoxy-1H-indol-2-yl) methyl) carbamate (3.60 g, 12.2 mmol, 1.00 eq) in DCM (40 mL) were added 2,6-lutidine (7.12 mL, 61.1 mmol, 5.00 eq) and TMSOTf (6.63 mL, 36.7 mmol, 3.00 eq) at 20° C. The mixture was stirred at 20° C. for 2 h. The mixture was concentrated under reduced pressure to give 5-fluoro-6-methoxy-1H-indol-2-yl) methanamine (Intermediate A-8) which was used without further purification. LC-MS (ESI) m/z: [M+H]⁺ calcd for C₁₀H₁₁FN₂O: 194.09; found: 178.2 [M−NH₂]⁺.

The following compounds in Table B-8 were synthesized using procedures similar to Intermediate A-8 using the appropriate starting materials.

TABLE B-8

| | | | Exact | LCMS, |
|---|---|---|---|---|
| Intermediate | | | mass | found |
| No. | Structure | IUPAC name | (g/mol) | [M + H]⁺ |
| B-8-1 | 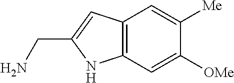 [SMILES: COc1cc2[nH]c(CN)cc2cc1C] | (6-methoxy-5- methyl-1H- indol-2- yl)methanamine | 190.11 | 191.1 |
| B-8-2 | 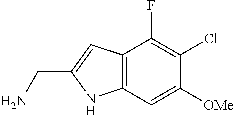 [SMILES: COc1cc2[nH]c(CN)cc2c(F)c1Cl] | (5-chloro-4- fluoro-6- methoxy-1H- indol-2- yl)methanamine | 228.05 | 229.3 |
| B-8-3 | 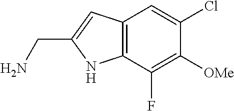 [SMILES: COc1c(Cl)cc2cc(CN)[nH]c2c1F] | (5-chloro-7- fluoro-6- methoxy-1H- indol-2- yl)methanamine | 228.05 | 212.1 [M − NH₂]⁺ |
| B-8-4 | 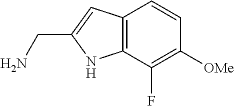 [SMILES: COc1ccc2cc(CN)[nH]c2c1F] | (7-fluoro-6- methoxy-1H- indol-2- yl)methanamine | 194.09 | 195.0 |
| B-8-5 | 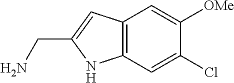 [SMILES: COc1cc2cc(CN)[nH]c2cc1Cl] | (6-chloro-5- methoxy-1H- indol-2- yl)methanamine | 210.06 | 211.1 |

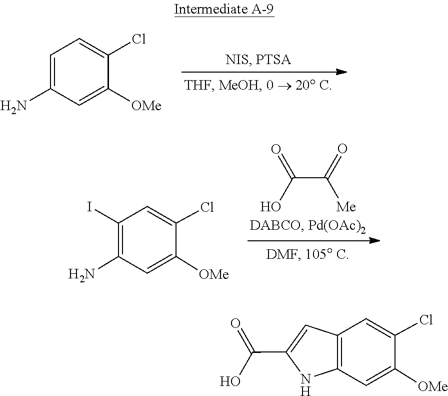 [SMILES: COc1cc(N)ccc1Cl ; COc1cc(N)c(I)cc1Cl ; CC(=O)C(=O)O ; COc1cc2[nH]c(C(=O)O)cc2cc1Cl]

### Step 1: 4-chloro-2-iodo-5-methoxyaniline

Three reactions were carried out in parallel as described. To a solution of 4-chloro-3-methoxyaniline (33.0 g, 209 mmol, 1.00 eq) in THF (330 mL) and MeOH (330 mL) were added N-iodosuccinimide (37.6 g, 167 mmol, 0.80 eq) and p-toluene sulfonic acid (36.0 g, 209 mmol, 1.00 eq) portion-wise at 0° C. The reaction mixture was then allowed to warm to 20° C. and stirred for 1 h, at which point the 3 reactions were combined for work up. The combined reaction mixture was poured into ice-water (500 mL) and stirred for 10 min. The aqueous phase was extracted with ethyl acetate (3×300 mL). The combined organic layers were washed with brine (500 mL), dried over anhydrous Na₂SO₄, filtered, and concentrated under reduced pressure to give a residue. The residue was purified by column chromatography to give 4-chloro-2-iodo-5-methoxyaniline. LC-MS (ESI) m/z calcd for C₇H₇ClINO: 282.93; found: 284.0 [M+H]⁺.

### Step 2: 5-chloro-6-methoxy-1H-indole-2-carboxylic acid

To a mixture of compound 4-chloro-2-iodo-5-methoxyaniline (40.0 g, 141 mmol, 1.00 eq), 2-oxopropanoic acid (37.2 g, 423 mmol, 3.00 eq) and DABCO (47.0 g, 423 mmol, 3.00 eq) in DMF (400 mL) was degassed and purged with N₂ 3 times, then Pd(OAc)₂ (3.17 g, 14.1 mmol, 0.10 eq) was added to the mixture in one portion at 25° C. under a N₂ atmosphere. The reaction mixture was then heated to 105° C. and stirred for 3 h. After cooling the mixture to room temperature, the reaction was poured into ice-water (500 mL) and stirred for 10 min. The aqueous phase was extracted with ethyl acetate (3×500 mL). The combined organic layers were washed with brine (500 mL), dried over anhydrous Na₂SO₄, filtered, and concentrated under reduced temperature to give a residue. The residue was triturated with MTBE at 20° C. for 30 min and filtered. The filter cake was collected to give 5-chloro-6-methoxy-1H-indole-2-carboxylic acid (Intermediate A-9). LC-MS (ESI) m/z calcd for C₁₀H₈ClNO₃: 225.02; found: 226.0 [M+H]⁺.

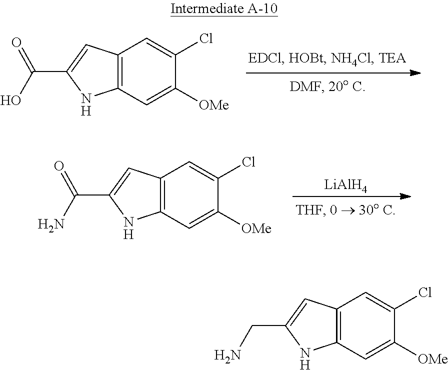 [SMILES: COc1cc2[nH]c(C(=O)O)cc2cc1Cl ; COc1cc2[nH]c(C(N)=O)cc2cc1Cl ; "LiAlH4" ; COc1cc2[nH]c(CN)cc2cc1Cl]

### Step 1: 5-chloro-6-methoxy-1H-indole-2-carboxamide

To a solution of 5-chloro-6-methoxy-1H-indole-2-carboxylic acid (15.8 g, 70 mmol, 1.00 eq) in DMF (160 mL) were added EDCI (16.1 g, 84 mmol, 1.20 eq) and HOBt (11.0 g, 84 mmol, 1.20 eq). The mixture was stirred at 20° C. for 0.5 h, then NH₄Cl (5.62 g, 105 mmol, 1.50 eq) and triethylamine (105 mmol, 14.6 mL, 1.50 eq) were added portion-wise. The reaction mixture was then stirred at 20° C. for 16 h, then was quenched with water (300 mL) and stirred for 10 min. The aqueous phase was extracted with ethyl acetate (3×100 mL). The combined organic layers were washed with brine (200 mL), dried over anhydrous Na₂SO₄, filtered, and concentrated under reduced pressure to give a residue. The residue was triturated with methyl tert-butyl ether (200 mL) at 20° C. for 30 min and filtered. The filter cake was collected to give 5-chloro-6-methoxy-1H-indole-2-carboxamide. LC-MS (ESI) m/z calcd for C₁₀H₉ClN₂O₂: 224.04; found: 224.9 [M+H]⁺.

### Step 2: (5-chloro-6-methoxy-1H-indol-2-yl) methanamine

Two reactions were carried out in parallel as described. To a solution of 5-chloro-6-methoxy-1H-indole-2-carboxamide (23.0 g, 102 mmol) in THF (700 mL) was added LiAlH₄ (31.1 g, 819 mmol, 8.00 eq) portion-wise and carefully at 0° C. under N₂. After the addition was complete, the mixture was allowed to warm to 30° C. and stirred for 16 h. After cooling, 2 reactions were combined for work up. The combined reaction mixture was cooled to 0° C. and quenched by dropwise addition of water (31 mL), followed by slow addition of 15% aq. NaOH (31 mL) and water (93 mL). The mixture was then allowed to warm to 20° C. and stir 0.5 h, then filtered to remove the precipitate. The filtrate was concentrated under reduced pressure before it was dissolved in water (30 mL) and ethyl acetate (60 mL). The pH was adjusted to 2 by addition of 6M HCl, and the mixture was extracted with ethyl acetate (3×60 mL). The aqueous phase was then adjusted to pH 11 by addition of 4 M NaOH, and the mixture was extracted with ethyl acetate (3×60 mL). The combined organic layers were washed with brine (100 mL), dried over Na₂SO₄, filtered, and concentrated under reduced pressure to give Intermediate A-10, (5-chloro-6-methoxy-1H-indol-2-yl) methanamine. LC-MS (ESI) m/z calcd for C₁₀H₁₁ClN₂O: 210.06; found: 211.1 [M+H]⁺.

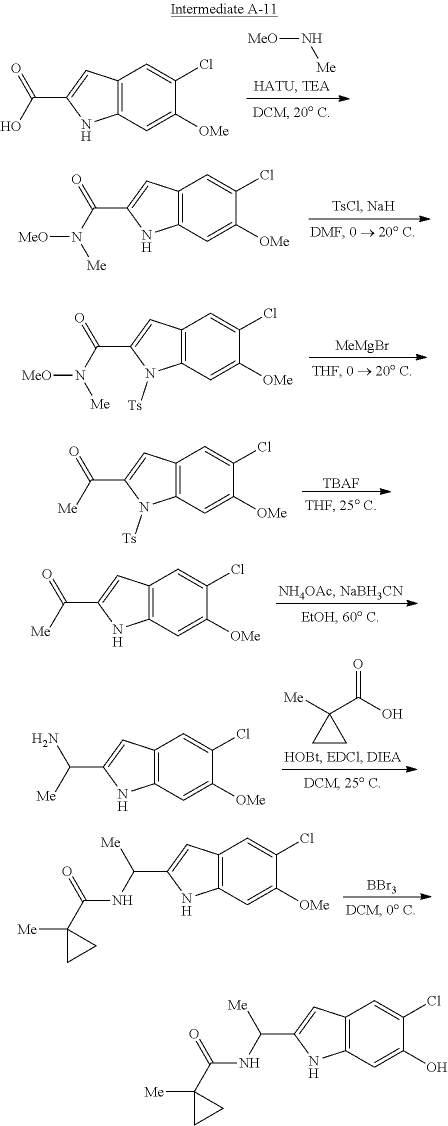

### Step 1: 5-chloro-N,6-dimethoxy-N-methyl-1H-indole-2-carboxamide

To a solution of 5-chloro-6-methoxy-1H-indole-2-carboxylic acid (5.00 g, 15.5 mmol, 70% purity, 1.00 eq) in DCM (50 mL) were added N-methoxymethanamine, HCl salt (2.27 g, 23.2 mmol, 1.50 eq, HCl), TEA (4.71 g, 46.5 mmol, 3.00 eq) and HATU (8.85 g, 23.2 mmol, 1.50 eq). The mixture was stirred for 0.5 h, then was quenched by addition of water (50 mL) and extracted with ethyl acetate (3×15 mL). The combined organic layers were washed with brine (3×15 mL), dried over Na₂SO₄, filtered, and concentrated under reduced pressure. The crude product was triturated with ethyl acetate to give 5-chloro-N,6-dimethoxy-N-methyl-1H-indole-2-carboxamide. LC-MS (ESI) m/z calcd for C₁₂H₁₃ClN₂O₃: 268.06; found: 269.1[M+H]⁺.

### Step 2: 5-chloro-N,6-dimethoxy-N-methyl-1-tosyl-1H-indole-2-carboxamide

To a solution of 5-chloro-N,6-dimethoxy-N-methyl-1H-indole-2-carboxamide (2.50 g, 9.30 mmol, 1.00 eq) in DMF (25 mL) at 0° C. was added NaH (446 mg, 11.1 mmol, 60% in mineral oil, 1.20 eq), and the mixture was stirred for 30 min. TsCl (2.13 g, 11.1 mmol, 1.20 eq) was then added, and the resulting mixture was stirred at 20° C. for 1 h. The reaction mixture cooled to 0° C. and quenched by addition of ice water (30 mL) and stirred for 2 min. The mixture was then extracted with ethyl acetate (3×10 mL). The combined organic layers were washed with brine (15 mL), dried over Na₂SO₄, filtered, and concentrated under reduced pressure. The crude product was triturated with ethyl acetate to give 5-chloro-N,6-dimethoxy-N-methyl-1-tosyl-1H-indole-2-carboxamide. LC-MS (ESI) m/z calcd for C₁₉H₁₉ClN₂O₅S: 422.07; found: 423.1 [M+H]⁺.

### Step 3: 1-(5-chloro-6-methoxy-1-tosyl-1H-indol-2-yl)ethan-1-one

To a solution of 5-chloro-N,6-dimethoxy-N-methyl-1-tosyl-1H-indole-2-carboxamide (1.50 g, 3.55 mmol, 1.00 eq) in THF (15 mL) at 0° C. was added MeMgBr (3 M in Et₂O, 11.8 mL, 35.4 mmol, 10.0 eq). The resulting mixture was stirred at 20° C. for 16 h. The reaction mixture was cooled to 0° C., quenched by addition of ice water (10 mL), and stirred for 2 min. The reaction mixture was then extracted with ethyl acetate (3×5 mL). The combined organic layers were washed with brine (5 mL), dried over Na₂SO₄, filtered, and concentrated under reduced pressure. The crude product was triturated with ethyl acetate to give 1-(5-chloro-6-methoxy-1-tosyl-1H-indol-2-yl)ethan-1-one (1.00 g). LC-MS (ESI) m/z calcd for C₁₈H₁₆ClNO₄S: 377.05; found: 378.1 [M+H]⁺.

### Step 4: 1-(5-chloro-6-methoxy-1H-indol-2-yl)ethan-1-one

To a solution of 1-(5-chloro-6-methoxy-1-tosyl-1H-indol-2-yl)ethan-1-one (1.00 g, 2.65 mmol, 1.00 eq) in THF (1 mL) was added TBAF (1 M in THF, 13.2 mL, 13.2 mmol, 5.00 eq). The mixture was stirred for 1 h, then was quenched by addition of water (10 mL) and extracted with ethyl acetate (3×5 mL). The combined organic layers were washed with sat. aq. NH₄Cl (3×5 mL), dried over Na₂SO₄, filtered, and concentrated under reduced pressure. The crude product was triturated with ethyl acetate to give 1-(5-chloro-6-methoxy-1H-indol-2-yl)ethan-1-one. LC-MS (ESI) m/z calcd for C₁₁H₁₀ClNO₂: 223.04; found: 224.1 [M+H]⁺.

### Step 5: 1-(5-chloro-6-methoxy-1H-indol-2-yl)ethan-1-amine

To a solution of 1-(5-chloro-6-methoxy-1H-indol-2-yl)ethan-1-one (330 mg, 1.48 mmol, 1.00 eq) in EtOH (16 mL) were added NH₄OAc (1.14 g, 14.7 mmol, 10.0 eq) and NaBH₃CN (463 mg, 7.38 mmol, 5.00 eq). The mixture was heated to 60° C. and stirred for 16 h. The reaction mixture was allowed to cool to RT, then was quenched by addition of water (20 mL) and extracted with ethyl acetate (3×20 mL). The combined organic layers were washed with brine (20 mL), dried over Na₂SO₄, filtered, and concentrated under reduced pressure. The crude product was triturated with ethyl acetate to give 1-(5-chloro-6-methoxy-1H-indol-2-yl)ethan-1-amine. LC-MS (ESI) m/z calcd for C₁₁H₁₃ClN₂O: 224.07; found: 208.2 [M−NH₂]⁺.

### Step 6: N-(1-(5-chloro-6-methoxy-1H-indol-2-yl)ethyl)-1-methylcyclopropane-1-carboxamide

To a solution of 1-methylcyclopropane-1-carboxylic acid (178 mg, 1.78 mmol, 1.00 eq) in DCM (4 mL) were added HOBt (264 mg, 1.96 mmol, 1.10 eq) and EDCI (375 mg, 1.96 mmol, 1.10 eq). The mixture was stirred for 0.5 h, then 1-(5-chloro-6-methoxy-1H-indol-2-yl)ethan-1-amine (400 mg, 1.78 mmol, 1.00 eq) and DIEA (460 mg, 3.56 mmol, 2.00 eq) were added. The resulting mixture was stirred for 1 h. The reaction mixture was cooled to 0° C., then was quenched by addition of H₂O (20 mL) and stirred for 2 min. The mixture was then extracted with ethyl acetate (3×10 mL). The combined organic layers were washed with brine (10 mL), dried over Na₂SO₄, filtered, and concentrated under reduced pressure. Purification by prep-HPLC gave N-(1-(5-chloro-6-methoxy-1H-indol-2-yl)ethyl)-1-methylcyclopropane-1-carboxamide. LC-MS (ESI) m/z calcd for C₁₆H₁₉ClN₂O₂: 306.11; found: 307.1 [M+H]⁺.

### Step 7: N-(1-(5-chloro-6-hydroxy-1H-indol-2-yl)ethyl)-1-methylcyclopropane-1-carboxamide

To a solution of N-(1-(5-chloro-6-methoxy-1H-indol-2-yl)ethyl)-1-methylcyclopropane-1-carboxamide (200 mg, 0.65 mmol, 1.00 eq) in DCM (2 mL) at 0° C. was added BBr₃ (653 mg, 2.61 mmol, 4.00 eq), and the resulting mixture was stirred at 0° C. for 3 h. The mixture was then diluted with water (20 mL) and adjusted to pH 7 by addition of sat. aq. NaHCO₃. The mixture was extracted with ethyl acetate (3×10 mL), the combined organic extracts were dried over Na₂SO₄, and concentrated under reduced pressure to give N-(1-(5-chloro-6-hydroxy-1H-indol-2-yl)ethyl)-1-methylcyclopropane-1-carboxamide (Intermediate A-11). LC-MS (ESI) m/z calcd for C₁₅H₁₇ClN₂O₂: 292.10; found: 293.2 [M+H]⁺.

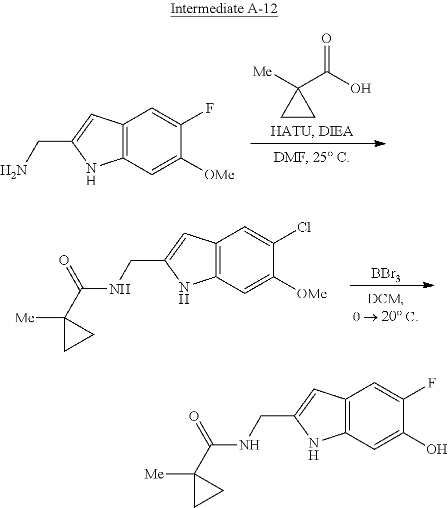 [SMILES: COc1cc2[nH]c(CN)cc2cc1F ; CC1(C(=O)O)CC1 ; COc1cc2[nH]c(CNC(=O)C3(C)CC3)cc2cc1Cl ; "BBr3" ; CC1(C(=O)NCc2cc3cc(F)c(O)cc3[nH]2)CC1]

### Step 1: N-((5-fluoro-6-methoxy-1H-indol-2-yl) methyl)-1-methylcyclopropane-1-carboxamide

To a solution of 1-methylcyclopropane-1-carboxylic acid (1.86 g, 18.5 mmol, 1.50 eq) in DMF (25 mL) was added HATU (7.05 g, 18.5 mmol, 1.50 eq), and the mixture was stirred at 20° C. for 1 h, whereupon (5-fluoro-6-methoxy-1H-indol-2-yl) methanamine (2.37 g, 12.3 mmol, 1.00 eq) and DIEA (6.46 mL, 37.1 mmol, 3.00 eq) as a solution in DMF (10 mL) was added. The reaction mixture was stirred at 20° C. for 3 h. Water (40 mL) was then added, and the resultant mixture was extracted with ethyl acetate (2×20 mL). The combined organic phases were washed with brine (40 mL), dried over anhydrous sodium sulfate, filtered, and concentrated under reduced pressure. The residue was purified by silica gel chromatography to give N-((5-fluoro-6-methoxy-1H-indol-2-yl) methyl)-1-methylcyclopropane-1-carboxamide. LC-MS (ESI) m/z calcd for C₁₅H₁₇FN₂O₂: 276.13; found: 277.2 [M+H]⁺.

### Step 2: N-((5-fluoro-6-hydroxy-1H-indol-2-yl) methyl)-1-methylcyclopropane-1-carboxamide

To a solution of N-((5-fluoro-6-methoxy-1H-indol-2-yl)methyl)-1-methylcyclopropane-1-carboxamide (1.80 g, 6.51 mmol, 1.00 eq) in DCM (30 mL) was added BBr₃ (2.51 mL, 26.1 mmol, 4.00 eq) dropwise at 0° C. The reaction mixture was stirred at 20° C. for 2 h. The reaction mixture was quenched by saturated aqueous NaHCO₃ solution (30 mL) and extracted with ethyl acetate (2×20 mL). The combined organic phases were washed with brine (20 mL), dried over anhydrous sodium sulfate, filtered, and concentrated under reduced pressure. The crude product was triturated with ethyl acetate (20 mL) at 20° C. for 20 mins then filtered to give N-((5-fluoro-6-hydroxy-H-indol-2-yl) methyl)-1-methylcyclopropane-1-carboxamide, Intermediate A-12. LC-MS (ESI) m/z calcd for C₁₄H₁₅FN₂O₂: 262.11; found: 263.1 [M+H]⁺.

The following compounds in Table B-12 were synthesized using procedures similar to Intermediate A-12 using the appropriate starting materials.

TABLE B-12

| Inter- | | | Exact | LCMS, |
|---|---|---|---|---|
| mediate | | | mass | found |
| No. | Structure | IUPAC name | (g/mol) | [M + H]⁺ |
| B-12-1 | 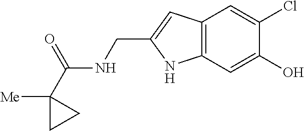 [SMILES: CC1(C(=O)NCc2cc3cc(Cl)c(O)cc3[nH]2)CC1] | N-((5-chloro-6- hydroxy-1H-indol-2- yl)methyl)-1- methylcyclopropane-1- carboxamide | 278.08 | 279.1 |
| B-12-2 | 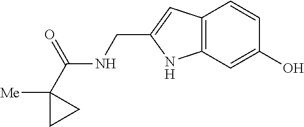 [SMILES: CC1(C(=O)NCc2cc3ccc(O)cc3[nH]2)CC1] | N-((6-hydroxy-1H- indol-2-yl)methyl)-1- methylcyclopropane-1- carboxamide | 244.12 | 245.1 |
| B-12-3 | 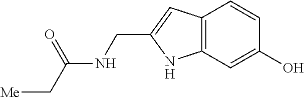 [SMILES: CCC(=O)NCc1cc2ccc(O)cc2[nH]1] | N-((6-hydroxy-1H- indol-2- yl)methyl)propionamide | 218.11 | 219.1 |
| B-12-4 | 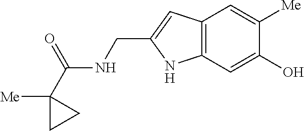 [SMILES: Cc1cc2cc(CNC(=O)C3(C)CC3)[nH]c2cc1O] | N-((6-hydroxy-5- methyl-1H-indol-2- yl)methyl)-1- methylcyclopropane-1- carboxamide | 258.14 | 259.2 |
| B-12-5 | 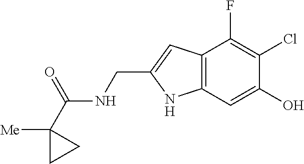 [SMILES: CC1(C(=O)NCc2cc3c(F)c(Cl)c(O)cc3[nH]2)CC1] | N-((5-chloro-4-fluoro- 6-hydroxy-1H-indol-2- yl)methyl)-1- methylcyclopropane-1- carboxamide | 296.07 | 297.2 |
| B-12-6 | 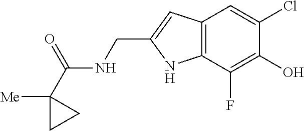 [SMILES: CC1(C(=O)NCc2cc3cc(Cl)c(O)c(F)c3[nH]2)CC1] | N-((5-chloro-7-fluoro- 6-hydroxy-1H-indol-2- yl)methyl)-1- methylcyclopropane-1- carboxamide | 296.07 | 297.1 |
| B-12-7 | 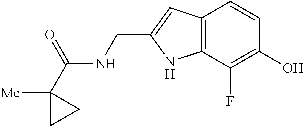 [SMILES: CC1(C(=O)NCc2cc3ccc(O)c(F)c3[nH]2)CC1] | N-((7-fluoro-6- hydroxy-1H-indol-2- yl)methyl)-1- methylcyclopropane-1- carboxamide | 262.11 | 263.1 |
| B-12-8 | 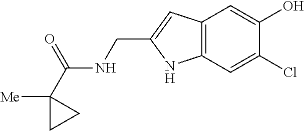 [SMILES: CC1(C(=O)NCc2cc3cc(O)c(Cl)cc3[nH]2)CC1] | N-((6-chloro-5- hydroxy-1H-indol-2- yl)methyl)-1- methylcyclopropane-1- carboxamide | 278.08 | 279.2 |
| B-12-9 | 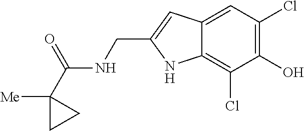 [SMILES: CC1(C(=O)NCc2cc3cc(Cl)c(O)c(Cl)c3[nH]2)CC1] | N-((5,7-dichloro-6- hydroxy-1H-indol-2- yl)methyl)-1- methylcyclopropane-1- carboxamide | 312.04 | 313.1 |

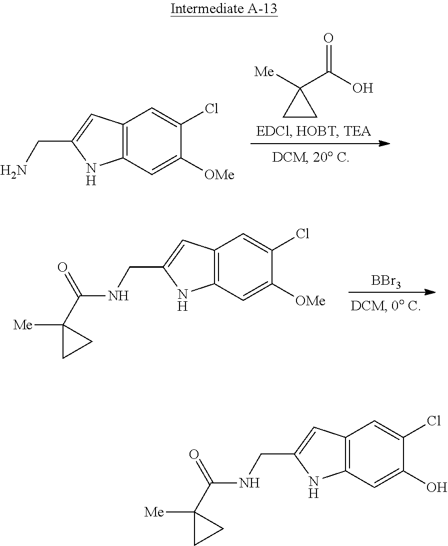 [SMILES: COc1cc2[nH]c(CN)cc2cc1Cl ; CC1(C(=O)O)CC1 ; COc1cc2[nH]c(CNC(=O)C3(C)CC3)cc2cc1Cl ; "BBr3" ; CC1(C(=O)NCc2cc3cc(Cl)c(O)cc3[nH]2)CC1]

### Step 1: N-((5-chloro-6-methoxy-1H-indol-2-yl)methyl)-1-methylcyclopropane-1-carboxamide

To a solution of 1-methylcyclopropanecarboxylic acid (5.13 g, 51.2 mmol, 1.20 eq) in DCM (100 mL) were added EDCI, 9.83 g, 51.2 mmol, 1.2 eq) and HOBt (6.93 g, 51.2 mmol, 1.2 eq). The mixture was stirred at 20° C. for 0.5 h under N₂, then triethylamine (128 mmol, 17.8 mL, 3.00 eq) and (5-chloro-6-methoxy-1H-indol-2-yl)methanamine (9.00 g, 42.7 mmol, 1.00 eq) were added portion-wise. The reaction mixture was then stirred at 20° C. for 1 h. The reaction mixture was quenched by addition of H₂O (200 mL) and then extracted with ethyl acetate (2×200 mL). The combined organic layers were washed with brine (150 mL), dried over Na₂SO₄, filtered, and concentrated under reduced pressure to give a residue. The residue was triturated with MTBE (80 mL) at 20° C. for 30 min and then filtered. The filter cake was collected to give N-((5-chloro-6-methoxy-1H-indol-2-yl)methyl)-1-methylcyclopropane-1-carboxamide. LC-MS (ESI) m/z calcd for C₁₅H₁₇ClN₂O₂: 292.10; found: 293.1 [M+H]⁺.

### Step 2: N-((5-chloro-6-hydroxy-1H-indol-2-yl)methyl)-1-methylcyclopropane-1-carboxamide

To a solution of N-((5-chloro-6-methoxy-1H-indol-2-yl)methyl)-1-methylcyclopropane-1-carboxamide (9.90 g, 33.8 mmol, 1.00 eq) in DCM (20 mL) was dropwise added BBr₃ (203 mmol 19.6 mL, 6.00 eq) at 0° C. under N₂. The mixture was stirred at 0° C. for 1 h, then was poured into ice-water (100 mL) and stirred for 5 mins. The aqueous phase was adjusted to pH 8 by addition of saturated aqueous NaHCO₃, then extracted with ethyl acetate (3×100 mL). The combined organic phases were washed with brine (100 mL), dried over anhydrous Na₂SO₄, filtered, and concentrated under reduced pressure. The residue was purified by silica gel column chromatography and then further purified by trituration with methanol (2×20 mL) at 20° C. for 15 min. The product was isolated by filtration to give N-((5-chloro-6-hydroxy-1H-indol-2-yl)methyl)-1-methylcyclopropane-1-carboxamide, Intermediate A-13. LC-MS (ESI) m/z calcd for C₁₄H₁₅ClN₂O₂: 278.08; found: 279.1 [M+H]⁺.

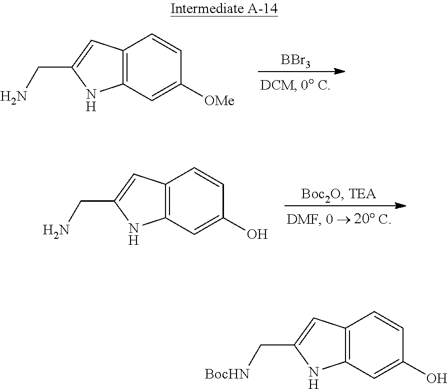 [SMILES: COc1ccc2cc(CN)[nH]c2c1 ; "BBr3" ; NCc1cc2ccc(O)cc2[nH]1 ; "Boc2O, TEA" ; CC(C)(C)OC(=O)NCc1cc2ccc(O)cc2[nH]1]

### Step 1. 2-(aminomethyl)-1H-indol-6-ol

Four reactions were carried out in parallel. To a solution of (6-methoxy-1H-indol-2-yl)methanamine (4.25 g, 24.1 mmol, 1.00 eq) in dichloromethane (150 mL) was drop-wise added BBr₃ (96.5 mmol, 9.3 mL, 4.00 eq) at 0° C., and the reaction mixture was maintained at 0° C. for 2 h. The four reactions were combined and poured into water (60 mL) carefully at 0° C., then adjusted to pH=7 with saturated aqueous NaHCO₃. The solution was extracted with ethyl acetate (3×150 mL). The combined organic phases were washed with brine (200 mL), dried over Na₂SO₄, filtered, and concentrated under reduced pressure to give 2-(aminomethyl)-1H-indol-6-ol, which was used without further purification. LC-MS (ESI) m/z calcd for C₉H₁₀N₂O: 162.08; found: 146.1 [M−NH₂]⁺.

### Step 2. tert-butyl ((6-hydroxy-1H-indol-2-yl)methyl)carbamate

To a solution of 2-(aminomethyl)-1H-indol-6-ol (9.5 g, 80% purity, 37.4 mmol, 1.00 eq) in N,N-dimethylformamide (50 mL) was added Boc₂O (37.5 mmol, 8.6 mL, 1.00 eq) and triethylamine (140.6 mmol, 19.6 mL, 3.75 eq) at 0° C. under N₂. After the addition, the reaction was allowed to warm to 20° C. and stirred for 2 h. The reaction mixture was then quenched by water (80 mL) at 20° C. and extracted with ethyl acetate (2×100 mL). The combined organic layers were washed with brine (2×100 mL), dried over anhydrous Na₂SO₄, filtered, and concentrated under reduced pressure to give a residue. The residue was purified by silica gel chromatography to give tert-butyl ((6-hydroxy-1H-indol-2-yl)methyl)carbamate (Intermediate A-14). LC-MS (ESI) m/z calcd for C₁₄H₁₈N₂O₃: 262.13; found: 263.1 [M+H]⁺; ¹H NMR (400 MHz, DMSO-d₆) δ 10.43 (s, 1H), 8.78 (s, 1H), 7.11-7.27 (m, 2H), 6.68 (d, J=1.6 Hz, 1H), 6.46 (dd, J=8.4, 2.0 Hz, 1H), 6.04 (s, 1H), 4.17 (d, J=5.6 Hz, 2H), 1.40 (s, 9H).

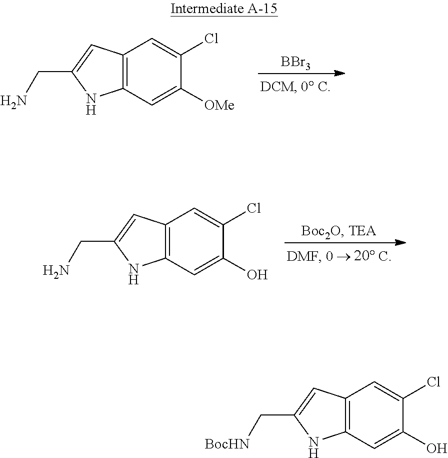 [SMILES: COc1cc2[nH]c(CN)cc2cc1Cl ; "BBr3" ; NCc1cc2cc(Cl)c(O)cc2[nH]1 ; "Boc2O, TEA" ; CC(C)(C)OC(=O)NCc1cc2cc(Cl)c(O)cc2[nH]1]

### Step 1: 2-(aminomethyl)-5-chloro-1H-indol-6-ol

To a solution of (5-chloro-6-methoxy-1H-indol-2-yl) methanamine (2.50 g, 11.9 mmol, 1.00 eq) in dichloromethane (50 mL) was added dropwise BBr₃ (47.5 mmol, 4.6 mL, 4.00 eq) at 0° C., and the reaction mixture was stirred at 0° C. for 2 h. The mixture was poured into water (15 mL) at 0° C. and adjusted to pH=7 by addition of saturated aqueous NaHCO₃, then extracted with ethyl acetate (3×20 mL). The combined organic phases were washed with brine (20 mL), dried over Na₂SO₄, filtered, and concentrated under reduced pressure to give 2-(aminomethyl)-5-chloro-1H-indol-6-ol, which was used without further purification. LC-MS (ESI) m/z calcd for C₉H₉ClN₂O: 196.04; found: 197.1[M+H]⁺.

### Step 2: tert-butyl ((5-chloro-6-hydroxy-1H-indol-2-yl)methyl) carbamate

To a solution of 2-(aminomethyl)-5-chloro-1H-indol-6-ol (2.30 g, 9.36 mmol, 80% purity) in DMF (20 mL) were added Boc₂O (7.49 mmol, 1.72 mL, 0.80 eq) and TEA (28.1 mmol, 3.90 mL, 3.00 eq) at 0° C. under N₂. The reaction mixture was then stirred at 20° C. for 1 h. The mixture was poured into water (20 mL) and stirred for 5 min. The aqueous phase was extracted with ethyl acetate (2×20 mL). The combined organic phases were washed with brine (2×20 mL), dried over anhydrous Na₂SO₄, filtered, and concentrated under reduced pressure. The residue was purified by silica gel chromatography to give tert-butyl ((5-chloro-6-hydroxy-1H-indol-2-yl)methyl) carbamate, Intermediate A-15. LC-MS (ESI) m/z calcd for C₁₄H₁₇ClN₂O₃: 296.09; found: 297.1 [M+H]⁺.

The following compounds in Table B-15 were synthesized using procedures similar to Intermediate A-15 using the appropriate starting materials.

TABLE B-15

| Inter- | | | Exact | LCMS, |
|---|---|---|---|---|
| mediate | | | mass | found |
| No. | Structure | IUPAC name | (g/mol) | [M + H]⁺ |
| B-15-1 | 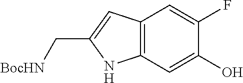 [SMILES: CC(C)(C)OC(=O)NCc1cc2cc(F)c(O)cc2[nH]1] | tert-butyl ((5-fluoro- 6-hydroxy-1H- indol-2- yl)methyl)carbamate | 280.12 | 281.4 |

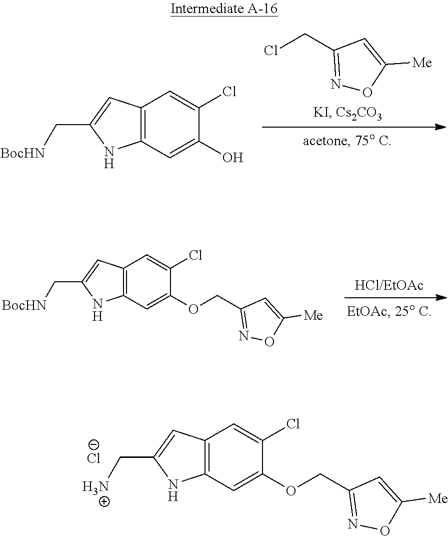 [SMILES: CC(C)(C)OC(=O)NCc1cc2cc(Cl)c(O)cc2[nH]1 ; Cc1cc(CCl)no1 ; Cc1cc(COc2cc3[nH]c(CNC(=O)OC(C)(C)C)cc3cc2Cl)no1 ; [Cl-] ; Cc1cc(COc2cc3[nH]c(C[NH3+])cc3cc2Cl)no1]

### Step 1: tert-butyl ((5-chloro-6-((5-methylisoxazol-3-yl)methoxy)-1H-indol-2-yl)methyl)carbamate

A mixture of tert-butyl ((5-chloro-6-hydroxy-1H-indol-2-yl)methyl)carbamate (3.00 g, 10.1 mmol, 1.00 eq), 3-(chloromethyl)-5-methyl-isoxazole (1.20 g, 9.10 mmol, 0.90 eq), Cs₂CO₃ (3.29 g, 10.1 mmol, 1.00 eq) and KI (168 mg, 1.01 mmol, 0.10 eq) in acetone (30 mL) was degassed and purged with N₂ 3 times, then was stirred at 75° C. for 16 h under N₂ atmosphere. The mixture was allowed to cool to room temperature, then was poured into water (50 mL) and extracted with EtOAc (2×50 mL). The organic layer was washed with brine (100 mL) and dried over Na₂SO₄, and concentrated under reduced pressure. The resultant residue was washed with MTBE (30 mL) then isolated by filtration to give tert-butyl ((5-chloro-6-((5-methylisoxazol-3-yl)methoxy)-1H-indol-2-yl)methyl)carbamate, which was used without further purification. LC-MS (ESI) m/z calcd for C₁₉H₂₂ClN₃O₄: 391.13; found: 392.3 [M+H]⁺.

### Step 2: (5-chloro-6-((5-methylisoxazol-3-yl)methoxy)-1H-indol-2-yl)methanamine hydrochloride

To a solution of tert-butyl ((5-chloro-6-((5-methylisoxazol-3-yl)methoxy)-1H-indol-2-yl)methyl)carbamate (2.78 g, 7.09 mmol, 1.00 eq) in EtOAc (20 mL) was added 4 M HCl in EtOAc (120 mmol, 30 mL, 16.9 eq) and the mixture was stirred at 20° C. for 1 h. The reaction mixture was filtered and the resulting solid was washed with MTBE (30 mL) then isolated by filtration to give (5-chloro-6-((5-methylisoxazol-3-yl)methoxy)-1H-indol-2-yl)methanamine hydrochloride, Intermediate A-16. LC-MS (ESI) m/z calcd for C₁₄H₁₄ClN₃O₂: 291.08; found: 275.1 [M−NH₂]⁺.

The following compounds in Table B-16 were synthesized using procedures similar to Intermediate A-16 using the appropriate starting materials.

TABLE B-16

| Inter- | | | Exact | LCMS, |
|---|---|---|---|---|
| mediate | | | mass | found |
| No. | Structure | IUPAC name | (g/mol) | [M + H]⁺ |
| B-16-1 | 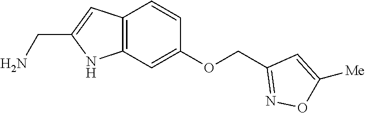 [SMILES: Cc1cc(COc2ccc3cc(CN)[nH]c3c2)no1] | (6-((5-methylisoxazol-3- yl)methoxy)-1H-indol-2- yl)methanamine | 257.12 | 241.1 [M − NH₂]⁺ |
| B-16-2 | 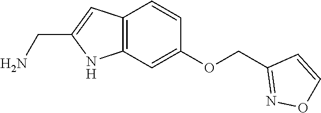 [SMILES: NCc1cc2ccc(OCc3ccon3)cc2[nH]1] | (6-(isoxazol-3- ylmethoxy)-1H-indol-2- yl)methanamine | 243.10 | 244.1 |
| B-16-3 | 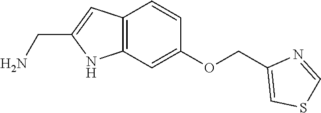 [SMILES: NCc1cc2ccc(OCc3cscn3)cc2[nH]1] | (6-(thiazol-4-ylmethoxy)- 1H-indol-2- yl)methanamine | 259.08 | 260.1 |
| B-16-4 | 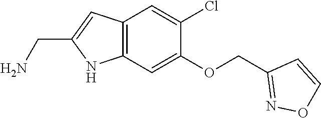 [SMILES: NCc1cc2cc(Cl)c(OCc3ccon3)cc2[nH]1] | (5-chloro-6-(isoxazol-3- ylmethoxy)-1H-indol-2- yl)methanamine | 277.06 | 261.1 [M − NH₂]⁺ |
| B-16-5 | 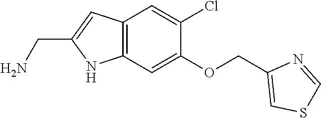 [SMILES: NCc1cc2cc(Cl)c(OCc3cscn3)cc2[nH]1] | (5-chloro-6-(thiazol-4- ylmethoxy)-1H-indol-2- yl)methanamine | 293.04 | 277.0 [M − NH₂]⁺ |
| B-16-6 | 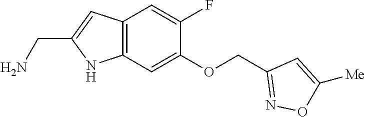 [SMILES: Cc1cc(COc2cc3[nH]c(CN)cc3cc2F)no1] | (5-fluoro-6-((5- methylisoxazol-3- yl)methoxy)-1H-indol-2- yl)methanamine | 275.11 | 259.1 [M − NH₂]⁺ |
| B-16-7 | 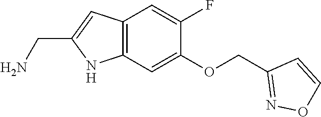 [SMILES: NCc1cc2cc(F)c(OCc3ccon3)cc2[nH]1] | (5-fluoro-6-(isoxazol-3- ylmethoxy)-1H-indol-2- yl)methanamine | 261.09 | 254.3 [M − NH₂]⁺ |
| B-16-8 | 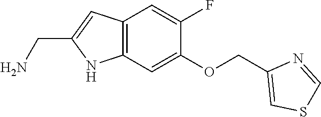 [SMILES: NCc1cc2cc(F)c(OCc3cscn3)cc2[nH]1] | (5-fluoro-6-(thiazol-4- ylmethoxy)-1H-indol-2- yl)methanamine | 277.07 | 261.2 [M − NH₂]⁺ |
| B-16-9 | 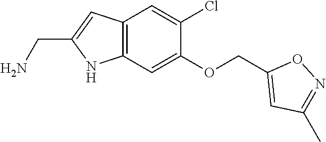 [SMILES: Cc1cc(COc2cc3[nH]c(CN)cc3cc2Cl)on1] | (5-chloro-6-((3- methylisoxazol-5- yl)methoxy)-1H-indol-2- yl)methanamine | 291.08 | 292.1 |
| B-16-10 | 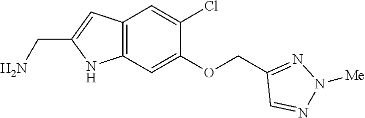 [SMILES: Cn1ncc(COc2cc3[nH]c(CN)cc3cc2Cl)n1] | (5-chloro-6-((2-methyl- 2H-1,2,3-triazol-4- yl)methoxy)-1H-indol-2- yl)methanamine | 291.09 | 275.1 [M − NH₂]⁺ |
| B-16-11 | 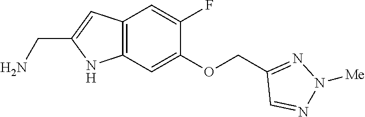 [SMILES: Cn1ncc(COc2cc3[nH]c(CN)cc3cc2F)n1] | (5-fluoro-6-((2-methyl- 2H-1,2,3-triazol-4- yl)methoxy)-1H-indol-2- yl)methanamine | 275.12 | 259.1 [M − NH₂]⁺ |
| B-16-12 | 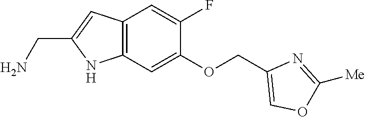 [SMILES: Cc1nc(COc2cc3[nH]c(CN)cc3cc2F)co1] | (5-fluoro-6-((2- methyloxazol-4- yl)methoxy)-1H-indol-2- yl)methanamine | 275.11 | 258.9 [M − NH₂]⁺ |

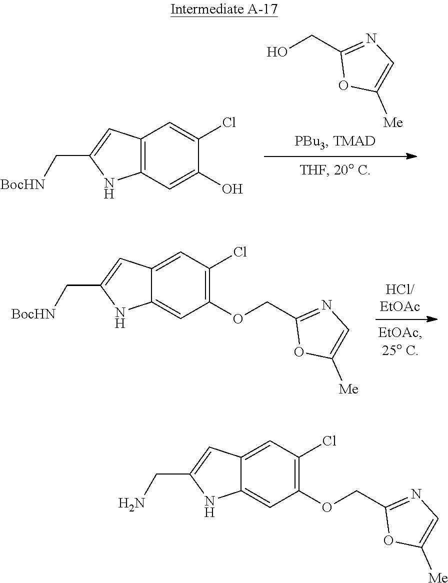 [SMILES: CC(C)(C)OC(=O)NCc1cc2cc(Cl)c(O)cc2[nH]1 ; Cc1cnc(CO)o1 ; Cc1cnc(COc2cc3[nH]c(CNC(=O)OC(C)(C)C)cc3cc2Cl)o1 ; Cc1cnc(COc2cc3[nH]c(CN)cc3cc2Cl)o1]

### Step 1: tert-butyl((5-chloro-6-((5-methyloxazol-2-yl)methoxy)-1H-indol-2-yl)methyl) carbamate

To a solution of tert-butyl ((5-chloro-6-hydroxy-1H-indol-2-yl)methyl)carbamate (550 mg, 1.85 mmol, 1.00 eq) and (5-methyloxazol-2-yl)methanol (489 mg, 2.59 mmol, 60% purity, 1.40 eq) in THF (15 mL) were added tributylphosphine (2.3 mL, 9.27 mmol, 5.00 eq) and N,N,N,N-tetramethylazodicarboxamide (1.60 g, 9.27 mmol, 5.00 eq). The mixture was stirred for 16 h. The reaction mixture was quenched by addition of water (10 mL) and extracted with EtOAc (2×5 mL). The combined organic phase was washed with brine (10 mL), dried over anhydrous sodium sulfate, filtered, and concentrated under reduced pressure. Purification by silica gel chromatography gave tert-butyl ((5-chloro-6-((5-methyloxazol-2-yl)methoxy)-1H-indol-2-yl)methyl)carbamate. LC-MS (ESI) m/z calcd for C₁₉H₂₂ClN₃O₄: 391.13; found: 392.3 [M+H]⁺.

### Step 2: (5-chloro-6-((5-methyloxazol-2-yl)methoxy)-1H-indol-2-yl)methanamine hydrochloride

A solution of tert-butyl ((5-chloro-6-((5-methyloxazol-2-yl)methoxy)-1H-indol-2-yl)methyl) carbamate (300 mg, 382 μmol, 50% purity, 1.00 eq) in HCl/EtOAc (4 M, 5 mL) was stirred at for 2 h. The mixture was concentrated under reduced pressure to give (5-chloro-6-((5-methyloxazol-2-yl)methoxy)-1H-indol-2-yl)methanamine hydrochloride (Intermediate A-17). LC-MS (ESI) m/z calcd for C₁₄H₁₄ClN₃O₂: 291.08; found: 292.2 [M+H]⁺.

The following compounds in Table B-17 were synthesized using procedures similar to Intermediate A-17 using the appropriate starting materials.

TABLE B-17

| Inter- | | | Exact | LCMS, |
|---|---|---|---|---|
| mediate | | | mass | found |
| No. | Structure | IUPAC name | (g/mol) | [M + H]⁺ |
| B-17-1 | 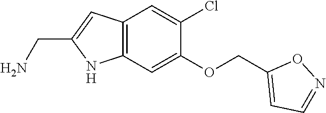 [SMILES: NCc1cc2cc(Cl)c(OCc3ccno3)cc2[nH]1] | (5-chloro-6- (isoxazol-5- ylmethoxy)-1H- indol-2- yl)methanamine | 277.06 | 261.1 [M − NH₂]⁺ |
| B-17-2 | 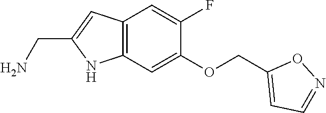 [SMILES: NCc1cc2cc(F)c(OCc3ccno3)cc2[nH]1] | (5-fluoro-6- (isoxazol-5- ylmethoxy)-1H- indol-2- yl)methanamine | 261.09 | 245.2 [M − NH₂]⁺ |
| B-17-3 | 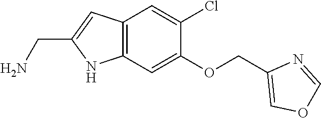 [SMILES: NCc1cc2cc(Cl)c(OCc3cocn3)cc2[nH]1] | (5-chloro-6- (oxazol-4- ylmethoxy)-1H- indol-2- yl)methanamine | 277.06 | 278.0 |
| B-17-4 | 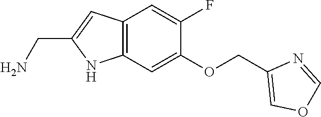 [SMILES: NCc1cc2cc(F)c(OCc3cocn3)cc2[nH]1] | (5-fluoro-6- (oxazol-4- ylmethoxy)-1H- indol-2- yl)methanamine | 261.09 | 245.1 [M − NH₂]⁺ |
| B-17-5 | 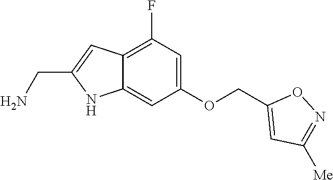 [SMILES: Cc1cc(COc2cc(F)c3cc(CN)[nH]c3c2)on1] | (4-fluoro-6-((3- methylisoxazol- 5-yl)methoxy)- 1H-indol-2- yl)methanamine | 275.11 | 245.1 [M − NH₂]⁺ |

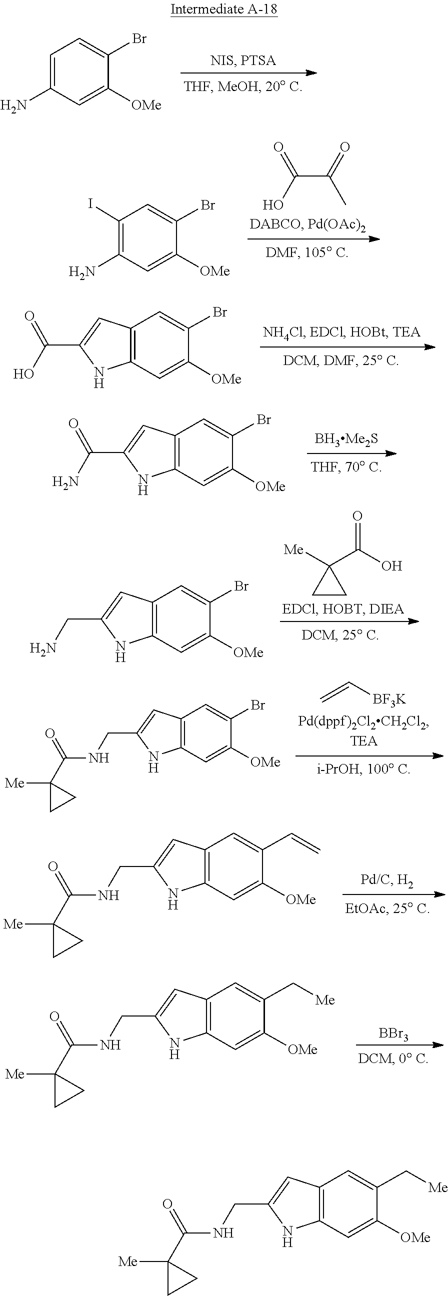

### Step 1: 4-bromo-2-iodo-5-methoxyaniline

Two parallel reactions were carried out. To a solution of 4-bromo-3-methoxyaniline (25.0 g, 124 mmol, 1.00 eq) in THF (250 mL) and MeOH (250 mL) at 0° C. were added NIS (22.3 g, 99.0 mmol, 0.80 eq) and 4-methylbenzenesulfonic acid (21.3 g, 124 mmol, 1.00 eq). The mixture was then stirred at 20° C. for 1 h. The two reactions were combined for worked up. The combined reaction mixture was poured into ice water (1 L) and stirred for 10 min. The aqueous phase was extracted with ethyl acetate (3×500 mL). The combined organic layers were washed with brine (1 L), dried over anhydrous Na₂SO₄, filtered, and concentrated under reduced pressure. Purification by column chromatography gave 4-bromo-2-iodo-5-methoxyaniline. LC-MS (ESI) m/z calcd for C₇H₇BrINO: 326.88; found: 327.9 [M+H]⁺.

### Step 2: 5-bromo-6-methoxy-1H-indole-2-carboxylic acid

To a solution of 4-bromo-2-iodo-5-methoxyaniline (32.8 g, 100 mmol, 1.00 eq) in DMF (164 mL) were added 2-oxopropanoic acid (26.4 g, 300 mmol, 21.1 mL, 3.00 eq), DABCO (33.7 g, 300 mmol, 33.0 mL, 3.00 eq) and Pd(OAc)₂ (2.25 g, 10.0 mmol, 0.10 eq). The mixture was heated to 105° C. and stirred for 5 h. After allowing the mixture to cool to room temperature, it was poured into ice water (500 mL) and stirred for 10 min. The aqueous phase was extracted with ethyl acetate (3×500 mL). The combined organic layers were washed with brine (500 mL), dried over anhydrous Na₂SO₄, filtered, and concentrated under reduced pressure. Purification by silica gel chromatography gave 5-bromo-6-methoxy-1H-indole-2-carboxylic acid. LC-MS (ESI) m/z calcd for C₁₀H₈BrNO₃: 268.97; found: 269.9 [M+H]⁺.

### Step 3: 5-bromo-6-methoxy-1H-indole-2-carboxamide

To a solution of 5-bromo-6-methoxy-1H-indole-2-carboxylic acid (13.6 g, 50.5 mmol, 1.00 eq) in DCM (140 mL) and DMF (28 mL) were added HOBt (8.18 g, 60.6 mmol, 1.20 eq) and EDCI (11.6 g, 60.6 mmol, 1.20 eq). The mixture was stirred for 30 min, then NH₄Cl (5.40 g, 101 mmol, 2.00 eq) and TEA (101 mmol, 14.1 mL, 2.00 eq) were added. The mixture was stirred for 16 h. The reaction was poured into ice-water (250 mL) and stirred for 10 min. The aqueous phase was extracted with ethyl acetate (3×100 mL). The combined organic layers were washed with brine (2×100 mL), dried over anhydrous Na₂SO₄, filtered, and concentrated under reduced pressure. The crude product was triturated with MTBE to give 5-bromo-6-methoxy-1H-indole-2-carboxamide. LC-MS (ESI) m/z calcd for C₁₀H₉BrN₂O₂: 267.98; found: 269.0 [M+H]⁺.

### Step 4: (5-bromo-6-methoxy-1H-indol-2-yl) methanamine

To a solution of 5-bromo-6-methoxy-1H-indole-2-carboxamide (11.4 g, 42.4 mmol, 1.00 eq) in THF (228 mL) was added BH₃-Me₂S (10 M in dimethylsulfide, 21.2 mL, 212 mmol, 5.00 eq). The mixture was stirred at 70° C. for 16 h. The reaction mixture was cooled to 0° C. and quenched by dropwise addition of MeOH (50 mL), then adjusted to pH 2 with 1N HCl and extracted with EtOAc (2×10 mL). The aqueous layer was adjusted to pH 8 with sat. aq. NaHCO₃, then extracted with EtOAc (3×100 mL). The combined organic layers were washed with brine (100 mL), dried over Na₂SO₄, filtered, and concentrated under reduced pressure. The crude product was triturated with MTBE to give (5-bromo-6-methoxy-1H-indol-2-yl) methanamine. LC-MS (ESI) m/z calcd for C₁₀H₁₁BrN₂O: 254.01; found: 238.0 [M−NH₂]⁺.

### Step 5: N-((5-bromo-6-methoxy-1H-indol-2-yl) methyl)-1-methylcyclopropanecarboxamide

To a solution of 1-methylcyclopropanecarboxylic acid (1.30 g, 13.0 mmol, 1.00 eq) in DCM (35 mL) were added EDCI (2.98 g, 15.5 mmol, 1.20 eq) and HOBt (2.10 g, 15.5 mmol, 1.20 eq). The mixture was stirred for 30 min, then (5-bromo-6-methoxy-1H-indol-2-yl) methanamine (3.30 g, 12.9 mmol, 1.00 eq) and TEA (38.8 mmol, 5.40 mL, 3.00 eq) were added. The resulting mixture was stirred for 16 h. The reaction mixture was quenched by addition of H₂O (50 mL), and then extracted with EtOAc (3×50 mL). The combined organic layers were washed with brine (50 mL), dried over Na₂SO₄, filtered, and concentrated under reduced pressure. Purification by column chromatography gave N-((5-bromo-6-methoxy-1H-indol-2-yl) methyl)-1-methylcyclopropanecarboxamide. LC-MS (ESI) m/z calcd for C₁₅H₁₇BrN₂O₂: 336.05; found: 337.0 [M+H]⁺.

### Step 6: N-((6-methoxy-5-vinyl-1H-indol-2-yl) methyl)-1-methylcyclopropane-1-carboxamide

A mixture of potassium vinyltrifluoroborate (500 mg, 3.74 mmol, 1.50 eq), N-((5-bromo-6-methoxy-1H-indol-2-yl)methyl)-1-methylcyclopropane-1-carboxamide (840 mg, 2.49 mmol, 1.00 eq), Pd(dppf)₂Cl₂·CH₂Cl₂ (203 mg, 249 μmol, 0.10 eq), TEA (1.04 mL, 7.47 mmol, 3.00 eq) in i-PrOH (10 mL) and was degassed and purged with N₂ three times, and then the mixture was stirred at 100° C. for 12 h under N₂ atmosphere. The reaction mixture was poured into ice-water (10 mL) and stirred for 2 min. The aqueous phase was extracted with ethyl acetate (3×20 mL). The combined organic phase was washed with brine (2×10 mL), dried with anhydrous Na₂SO₄, filtered, and concentrated under reduced pressure. Purification by column chromatography gave N-((6-methoxy-5-vinyl-1H-indol-2-yl) methyl)-1-methylcyclopropane-1-carboxamide. LC-MS (ESI) m/z calcd for C₁₇H₂₀N₂O₂: 284.15; found: 285.2 [M+H]⁺.

### Step 7: N-((5-ethyl-6-methoxy-1H-indol-2-yl) methyl)-1-methylcyclopropane-1-carboxamide

Three reactions were carried out in parallel. To a solution of N-((6-methoxy-5-vinyl-1H-indol-2-yl)methyl)-1-methylcyclopropane-1-carboxamide (150 mg, 527 μmol, 1.00 eq) in EtOAc (1 mL) was added Pd/C (100 mg, 528 μmol, 10% w/w, 1.00 eq) under N₂, then the suspension was degassed under vacuum and purged with H₂ several times. The mixture was then stirred under H₂ (15 psi) for 0.5 h. Three reactions were combined and filtered through Celite. The filtrate was concentrated under reduced pressure. Purification by prep-TLC gave N-((5-ethyl-6-methoxy-1H-indol-2-yl) methyl)-1-methylcyclopropane-1-carboxamide. LC-MS (ESI) m/z calcd for C₁₇H₂₂N₂O₂: 286.17; found: 287.3 [M+H]⁺.

### Step 8: N-((5-ethyl-6-hydroxy-1H-indol-2-yl) methyl)-1-methylcyclopropane-1-carboxamide

To a solution of N-((5-ethyl-6-methoxy-1H-indol-2-yl) methyl)-1-methylcyclopropane-1-carboxamide (250 mg, 873 μmol, 1.00 eq) in DCM (3 mL) at 0° C. was added BBr₃ (3.49 mmol, 336 μL, 4.00 eq) dropwise under N₂. The mixture was stirred at 0° C. for 2 h. The reaction mixture was poured into ice water (5 mL) and stirred for 2 min. The aqueous phase was adjusted to pH 8 by addition of sat. aq. NaHCO₃ and extracted with ethyl acetate (3×10 mL). The combined organic phase was washed with brine (10 mL), dried over anhydrous Na₂SO₄, filtered, and concentrated under reduced pressure. Purification by prep-TLC gave N-((5-ethyl-6-hydroxy-1H-indol-2-yl) methyl)-1-methylcyclopropane-1-carboxamide (Intermediate A-18). LC-MS (ESI) m/z calcd for C₁₆H₂₀N₂O₂: 272.15; found: 273.3 [M+H]⁺.

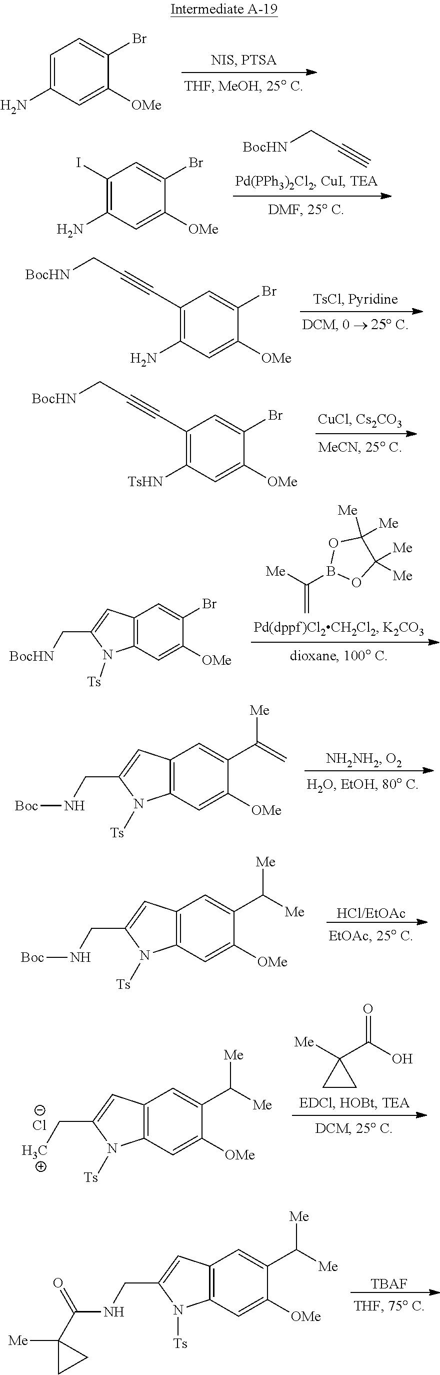

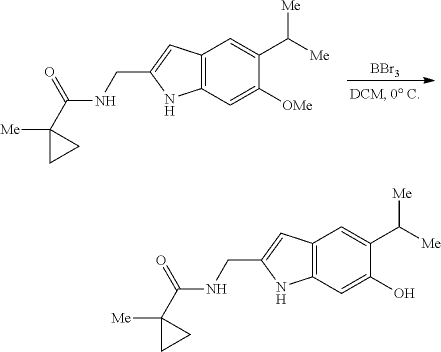

### Step 1: 4-bromo-2-iodo-5-methoxyaniline

Two reactions were carried out in parallel. To a solution of 4-bromo-3-methoxyaniline (50.0 g, 247 mmol, 1.00 eq) in THF (250 mL) and MeOH (250 mL) were added NIS (44.5 g, 198 mmol, 0.80 eq) and PTSA (42.6 g, 247 mmol, 1.00 eq). The mixture was stirred for 2 h. The two reactions were combined, poured into water (500 mL), and stirred for 5 min. The aqueous phase was extracted with ethyl acetate (3×1 L). The combined organic phase was washed with brine (2×1 L), dried over anhydrous Na₂SO₄, filtered, and concentrated under reduced pressure. Purification by column chromatography gave 4-bromo-2-iodo-5-methoxyaniline. LC-MS (ESI) m/z calcd for C₇H₇BrINO: 326.88; found: 327.9 [M+H]⁺.

### Step 2: tert-butyl (3-(2-amino-5-bromo-4-methoxyphenyl)prop-2-yn-1-yl)carbamate

2 reactions were carried out in parallel: To a solution of tert-butyl prop-2-yn-1-ylcarbamate (18.0 g, 116 mmol, 1.00 eq) and 4-bromo-2-iodo-5-methoxyaniline (38.0 g, 116 mmol, 1.00 eq) in DMF (400 mL) were added TEA (58.6 g, 579 mmol, 80.6 mL, 5.00 eq), CuI (2.21 g, 11.6 mmol, 0.10 eq) and dichlorobis(triphenylphosphine)palladium(II) (4.00 g, 5.79 mmol, 0.05 eq) under N₂, then the mixture was stirred for 12 h. The two reactions were combined, poured into water (400 mL), and stirred for 5 min. The aqueous phase was extracted with ethyl acetate (3×800 mL). The combined organic phase was washed with brine (2×500 mL), dried over anhydrous Na₂SO₄, filtered, and concentrated under reduced pressure. Purification by column chromatography gave tert-butyl (3-(2-amino-5-bromo-4-methoxyphenyl)prop-2-yn-1-yl)carbamate. LC-MS (ESI) m/z calcd for C₁₅H₁₉BrN₂O₃: 354.06; found: 299.1 [M−t-Bu]⁺.

### Step 3: tert-butyl (3-(5-bromo-4-methoxy-2-((4-methylphenyl)sulfonamido)phenyl)prop-2-yn-1-yl)carbamate

To a mixture of tert-butyl (3-(2-amino-5-bromo-4-methoxyphenyl)prop-2-yn-1-yl)carbamate (48.0 g, 135 mmol, 1.00 eq) and pyridine (21.4 g, 270 mmol, 21.8 mL, 2.00 eq) in DCM (480 mL) at 0° C. was added 4-methylbenzenesulfonyl chloride (28.3 g, 149 mmol, 1.10 eq). The mixture was then stirred at 25° C. for 16 h. The reaction mixture was quenched by addition of H₂O (300 mL), and then extracted with ethyl acetate (3×500 mL). The combined organic layers were washed brine (300 mL), dried over Na₂SO₄, filtered, and concentrated under reduced pressure. The residue was triturated with methyl tert-butyl ether (20 mL) to give tert-butyl (3-(5-bromo-4-methoxy-2-((4-methylphenyl)sulfonamido)phenyl)prop-2-yn-1-yl)carbamate. LC-MS (ESI) m/z: calcd for C₂₂H₂₅BrN₂O₅S: 508.07; found: 453.1 [M−t-Bu+H]⁺.

### Step 4: tert-butyl ((5-bromo-6-methoxy-1-tosyl-1H-indol-2-yl)methyl)carbamate

To a solution of tert-butyl (3-(5-bromo-4-methoxy-2-((4-methylphenyl)sulfonamido)phenyl)prop-2-yn-1-yl)carbamate (48.0 g, 94.0 mmol, 1.00 eq) in MeCN (500 mL) were added CuCl (1.40 g, 14.1 mmol, 338 μL, 0.15 eq) and Cs₂CO₃ (4.61 g, 14.1 mmol, 0.15 eq). The mixture was stirred for 12 h under N₂. The reaction mixture was poured into ice water (20 mL) and stirred for 2 min. The aqueous phase was extracted with ethyl acetate (3×1 L). The combined organic phase was washed with brine (2×1 L), dried over anhydrous Na₂SO₄, filtered, and concentrated under reduced pressure. Purification by column chromatography gave tert-butyl ((5-bromo-6-methoxy-1-tosyl-1H-indol-2-yl)methyl)carbamate. LC-MS (ESI) m/z calcd for C₂₂H₂₅BrN₂O₅S: 508.07; found: 531.0 [M+Na]⁺.

### Step 5: tert-butyl ((6-methoxy-5-(prop-1-en-2-yl)-1-tosyl-1H-indol-2-yl)methyl)carbamate

A mixture of tert-butyl ((5-bromo-6-methoxy-1-tosyl-1H-indol-2-yl)methyl)carbamate (1.40 g, 2.75 mmol, 1.00 eq), vinylboronic acid pinacol ester (923 mg, 5.50 mmol, 2.00 eq), and K₂CO₃ (949 mg, 6.87 mmol, 2.50 eq) in dioxane (16 mL) and H₂O (4 mL) was degassed and purged with N₂ 3 times, then added Pd(dppf)Cl₂·CH₂Cl₂ (224 mg, 274 μmol, 0.10 eq) and then the mixture was heated to 100° C. and stirred for 16 h under N₂. The reaction mixture was allowed to cool to RT, then was diluted with H₂O (20 mL) and extracted with ethyl acetate (3×20 mL). The combined organic layers were washed with brine (20 mL), dried over Na₂SO₄, filtered, and concentrated under reduced pressure. Purification by column chromatography gave tert-butyl ((6-methoxy-5-(prop-1-en-2-yl)-1-tosyl-1H-indol-2-yl)methyl)carbamate. LC-MS (ESI) m/z calcd for C₂₅H₃₀N₂O₅S: 470.19; found: 471.1 [M+H]⁺.

### Step 6: tert-butyl ((5-isopropyl-6-methoxy-1-tosyl-1H-indol-2-yl)methyl)carbamate

To a solution of tert-butyl ((6-methoxy-5-(prop-1-en-2-yl)-1-tosyl-1H-indol-2-yl)methyl)carbamate (900 mg, 1.91 mmol, 1.00 eq) in EtOH (10 mL) was added NH₂NH₂—H₂O (3.04 g, 48.5 mmol, 2.95 mL, 80% w/w, 25.4 eq) under O₂ (15 psi). The mixture was heated to 80° C. and stirred for 4 h. The reaction mixture was allowed to cool to room temperature, then was quenched by addition of H₂O (15 mL) and extracted with ethyl acetate (3×15 mL). The combined organic layers were washed with brine (15 mL), dried over Na₂SO₄, filtered, and concentrated under reduced pressure. Purification by column chromatography gave tert-butyl ((5-isopropyl-6-methoxy-1-tosyl-1H-indol-2-yl)methyl)carbamate. LC-MS (ESI) m/z calcd for C₂₅H₃₂N₂O₅S: 472.2; found: 495.1 [M+Na]⁺.

### Step 7: (5-isopropyl-6-methoxy-1-tosyl-1H-indol-2-yl)methanamine hydrochloride

To a solution of tert-butyl ((5-isopropyl-6-methoxy-1-tosyl-1H-indol-2-yl)methyl)carbamate (760 mg, 1.61 mmol, 1.00 eq) in ethyl acetate (8 mL) was added HCl (4 M in ethyl acetate, 10 mL, 40 mmol, 24.8 eq). The mixture was stirred for 2 h. The reaction mixture was concentrated in vacuo to give (5-isopropyl-6-methoxy-1-tosyl-1H-indol-2-yl)methanamine hydrochloride. LC-MS (ESI) m/z calcd for C₂₀H₂₄N₂O₃S: 372.15; found: 356.1 [M−NH₃]⁺.

### Step 8: N-((5-isopropyl-6-methoxy-1-tosyl-1H-indol-2-yl)methyl)-1-methylcyclopropane-1-carboxamide

To a solution of 1-methylcyclopropane-1-carboxylic acid (209 mg, 2.09 mmol, 1.20 eq) in DCM (8 mL) were added EDCI (401 mg, 2.09 mmol, 1.20 eq) and HOBt (282 mg, 2.09 mmol, 1.20 eq). The mixture was stirred for 0.5 h under N₂, then TEA (529 mg, 5.24 mmol, 728 μL, 3.00 eq) and (5-isopropyl-6-methoxy-1-tosyl-1H-indol-2-yl)methanamine hydrochloride (650 mg, 1.75 mmol, 1.00 eq) were added portionwise. The reaction mixture was stirred for 1 h, then was quenched by addition of H₂O (20 mL) and extracted with ethyl acetate (3×20 mL). The combined organic layers were washed with brine (30 mL), dried over Na₂SO₄, filtered, and concentrated under reduced pressure. Purification by column chromatography gave N-((5-isopropyl-6-methoxy-1-tosyl-1H-indol-2-yl)methyl)-1-methylcyclopropane-1-carboxamide. LC-MS (ESI) m/z calcd for C₂₅H₃₀N₂O₄S: 454.19; found: 455.1 [M+H]⁺.

### Step 9: N-((5-isopropyl-6-methoxy-1H-indol-2-yl)methyl)-1-methylcyclopropane-1-carboxamide

To a solution of N-((5-isopropyl-6-methoxy-1-tosyl-1H-indol-2-yl)methyl)-1-methylcyclopropane-1-carboxamide (630 mg, 1.39 mmol, 1.00 eq) in THF (6 mL) was added TBAF (1 M in THF, 4.16 mL, 4.16 mmol, 3.00 eq). The mixture was then heated to 75° C. and stirred for 16 h. The reaction mixture was allowed to cool to room temperature, then was quenched by addition of H₂O (15 mL) and extracted with ethyl acetate (3×15 mL). The combined organic layers were washed with brine (20 mL), dried over Na₂SO₄, filtered, and concentrated under reduced pressure. Purification by column chromatography gave N-((5-isopropyl-6-methoxy-1H-indol-2-yl)methyl)-1-methylcyclopropane-1-carboxamide. LC-MS (ESI) m/z calcd for C₁₈H₂₄N₂O₂: 300.18; found: 301.1 [M+H]⁺.

### Step 10: N-((6-hydroxy-5-isopropyl-1H-indol-2-yl)methyl)-1-methylcyclopropane-1-carboxamide

A mixture of N-((5-isopropyl-6-methoxy-1H-indol-2-yl)methyl)-1-methylcyclopropane-1-carboxamide (340 mg, 1.13 mmol, 1.00 eq) in DCM (5 mL) at 0° C. was added BBr₃ (1.42 g, 5.66 mmol, 545 μL, 5.00 eq), and the resulting mixture was stirred at 0° C. for 2 h under N₂ atmosphere. The reaction mixture was quenched at 0° C. by addition of sat. aq. NaHCO₃, then was extracted with ethyl acetate (3×10 mL). The combined organic layers were washed with brine (20 mL), dried over Na₂SO₄, filtered, and concentrated under reduced pressure. Purification by column chromatography gave N-((6-hydroxy-5-isopropyl-1H-indol-2-yl)methyl)-1-methylcyclopropane-1-carboxamide (Intermediate A-19). LC-MS (ESI) m/z calcd for C₁₈H₂₄N₂O₂: 286.17; found: 287.2 [M+H]⁺.

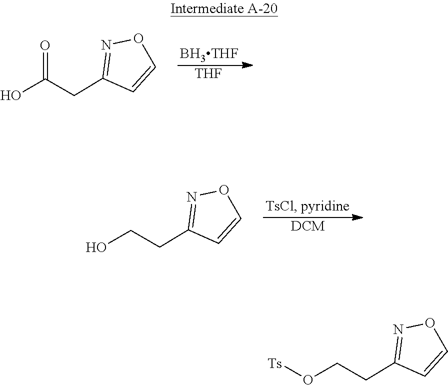

### Step 1: 2-(isoxazole-3-yl)ethan-1-ol

To a solution of BH₃-THF (3.90 mL, 1 M, 3.90 mmol, 2.00 eq) in THF (4 mL) was added 2-(isoxazole-3-yl)acetic acid (248 mg, 1.95 mmol, 1.00 eq) under N₂, and the resulting solution stirred for 18 h. The reaction was concentrated before MeOH was added. The resulting solution was concentrated under reduced pressure to give 2-(isoxazole-3-yl)ethan-1-ol, which was used in next step without further purification.

### Step 2: 2-(isoxazole-3-yl)ethyl 4-methylbenzenesulfonate

To a solution of 2-(isoxazole-3-yl)ethan-1-ol (221 mg, 1.95 mmol, 1.00 eq) in DCM (4 mL) were added pyridine (198 uL, 2.34 mmol, 1.20 eq) and TsCl (409 mg, 2.15 mmol, 1.10 eq), and the solution was stirred at room temperature for 1 h. The reaction was quenched by addition of water (5 mL) and extracted with DCM (3×5 mL). The combined organic phase was washed with brine (5 mL), dried over anhydrous Na₂SO₄, filtered, and concentrated in vacuo to give 2-(isoxazole-3-yl)ethyl 4-methylbenzenesulfonate (Intermediate A-20) which was used in next step without further purification. LC-MS (ESI) m/z calcd for C₁₂H₁₃NO₄S: 267.1; found 268.1 [M+H]⁺.

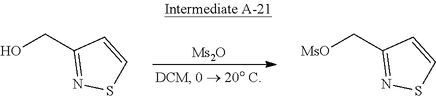 [SMILES: "Ms2O" ; OCc1ccsn1 ; CS(=O)(=O)OCc1ccsn1]

### Step 1. Isothiazol-3-ylmethyl methanesulfonate

To a solution of isothiazol-3-ylmethanol (300 mg, 2.61 mmol, 1.00 eq) in DCM (10 mL) was added TEA (527 mg, 5.21 mmol, 2.00 eq) and Ms₂O (681 mg, 3.91 mmol, 1.50 eq) at 0° C. The reaction mixture was stirred at 20° C. for 2 h. The reaction mixture was diluted with water (30 mL) and extracted with DCM (3×10 mL). The combined organic layers were washed with brine (10 mL), dried over Na₂SO₄, filtered, and concentrated under reduced pressure to give isothiazol-3-ylmethyl methanesulfonate (Intermediate A-21), which was used without further purification.

The following compounds in Table B-21 were synthesized using procedures similar to Intermediate A-21 using the appropriate starting materials.

| Inter- | | | Exact | LCMS, |
|---|---|---|---|---|
| mediate | | | mass | found |
| No. | Structure | IUPAC name | (g/mol) | [M + H]⁺ |
| B-21-1 | 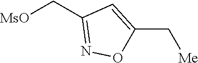 [SMILES: CCc1cc(COS(C)(=O)=O)no1] | (5-ethylisoxazol-3-yl)methyl methanesulfonate | 205.04 | 206.1 |
| B-21-2 | 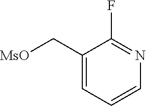 [SMILES: CS(=O)(=O)OCc1cccnc1F] | (2-fluoropyridin-3-yl)methyl methanesulfonate | 205.02 | 206.1 |
| B-21-3 | 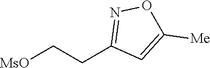 [SMILES: Cc1cc(CCOS(C)(=O)=O)no1] | 2-(5-methylisoxazol-3-yl)ethyl methanesulfonate | 205.04 | 206.1 |
| B-21-4 | 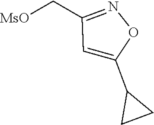 [SMILES: CS(=O)(=O)OCc1cc(C2CC2)on1] | (5-cyclopropylisoxazol-3-yl)methyl methanesulfonate | 217.04 | 218.1 |
| B-21-5 | 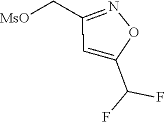 [SMILES: CS(=O)(=O)OCc1cc(C(F)F)on1] | (5-(difluoromethyl)isoxazole-3- yl)methyl methanesulfonate | 227.01 | 228.1 |
| B-21-6 | 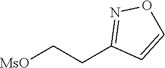 [SMILES: CS(=O)(=O)OCCc1ccon1] | 2-(isoxazole-3-yl)ethyl methanesulfonate | 191.03 | 192.2 |

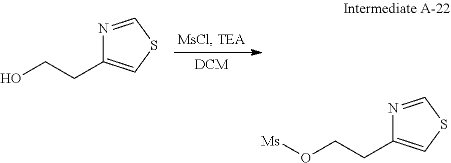 [SMILES: OCCc1cscn1 ; CS(=O)(=O)OCCc1cscn1]

### 2-(thiazol-4-yl)ethyl methanesulfonate

To a solution of 2-(thiazol-4-yl)ethan-1-ol (151 mg, 1.17 mmol, 1.00 eq) in DCM (3 mL) were added TEA (163 μL, 1.17 mmol, 1.00 eq) and MsCl (161 mg, 1.40 mmol, 1.20 eq). The reaction mixture was stirred at RT for 18 h. The reaction mixture was diluted with water (30 mL) and extracted with DCM (3×10 mL). The combined organic layers were washed with brine (10 mL), dried over Na₂SO₄, filtered, and concentrated under reduced pressure to give 2-(thiazol-4-yl)ethyl methanesulfonate (Intermediate A-22), which was used without further purification.

The following compounds in Table B-22 were synthesized using procedures similar to Intermediate A-22 using the appropriate starting materials.

TABLE B-22

| | | | Exact | LCMS, |
|---|---|---|---|---|
| | | | mass | found |
| Compound No. | Structure | IUPAC name | (g/mol) | [M + H]⁺ |
| B-22-1 | 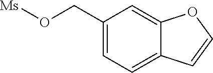 [SMILES: CS(=O)(=O)OCc1ccc2ccoc2c1] | benzofuran-6- ylmethyl methanesulfonate | 226.0 | N/A* |
| B-22-2 | 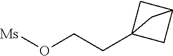 [SMILES: CS(=O)(=O)OCCC12CC(C1)C2] | 2- (bicyclo[1.1.1]pentan- 1-yl)ethyl methanesulfonate | 190.1 | N/A* |
| B-22-3 | 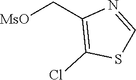 [SMILES: CS(=O)(=O)OCc1ncsc1Cl] | (5-chlorothiazol-4- yl)methyl methanesulfonate | 226.95 | N/A* |
| B-22-4 | 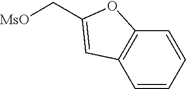 [SMILES: CS(=O)(=O)OCc1cc2ccccc2o1] | benzofuran-2- ylmethyl methanesulfonate | 226.03 | N/A* |

*No ionization observed in LCMS

### Step 1. 3-((chlorotriphenyl-λ5-phosphanyl)methyl)isoxazole

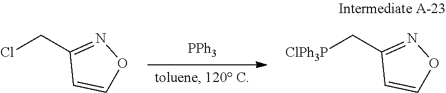

To a solution of 3-(chloromethyl)isoxazole (400 mg, 3.40 mmol, 1.00 eq) in toluene (5 mL) was added PPh₃ (1.21 g, 4.59 mmol, 1.35 eq). The reaction mixture was stirred at 120° C. for 7 h. The reaction mixture was filtered and concentrated under reduced pressure to give a residue which was triturated with MTBE (5 mL) at 25° C. for 20 min to give 3-((chlorotriphenylphosphoranyl)methyl)isoxazole, Intermediate A-23.

The following compounds in Table B-23 were synthesized using procedures similar to Intermediate A-23 using the appropriate starting materials.

TABLE B-23

| Intermediate | | | Exact mass | LCMS, found |
|---|---|---|---|---|
| No. | Structure | IUPAC name | (g/mol) | [M]⁺ |
| B-23-1 | 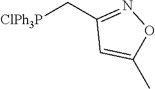 | 3-((chlorotriphenyl-λ5- phosphaneyl)methyl)- 5-methylisoxazole | 358.14 | 358.2 |

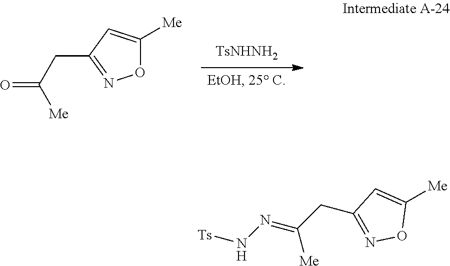 [SMILES: "TsNHNH2" ; CC(=O)Cc1cc(C)on1 ; "EtOH, 25° C." ; C/C(Cc1cc(C)on1)=N\NS(=O)(=O)c1ccc(C)cc1]

### Step 1: 4-methyl-N′-(1-(5-methylisoxazol-3-yl) propan-2-ylidene) benzenesulfonohydrazide

To a mixture of 1-(5-methylisoxazol-3-yl) propan-2-one (66.9 mg, 359 μmol, 1.00 eq) in EtOH (5 mL) was added p-toluenesulfonyl hydrazide (50.0 mg, 359 μmol, 1.00 eq) in one portion at 25° C. under N₂ and the resulting mixture was stirred for 4 h. The reaction mixture was quenched by addition of H₂O (30 mL) and extracted with ethyl acetate (3×10 mL). The combined organic layers were dried over Na₂SO₄, filtered, and concentrated under reduced pressure to give a residue. The residue was purified by silica gel chromatography to give 4-methyl-N′-(1-(5-methylisoxazol-3-yl) propan-2-ylidene) benzenesulfonohydrazide, Intermediate A-24. LC-MS (ESI) m/z calcd for C₁₄H₁₇N₃O₃S: 307.1; found 308.1 [M+H]⁺.

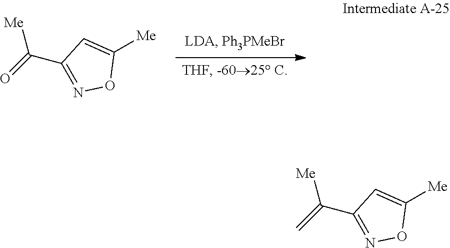

### Step 1: 5-methyl-3-(prop-1-en-2-yl)isoxazole

To a solution of methyl(triphenyl)phosphonium bromide (12.8 g, 35.9 mmol, 1.50 eq) in THF (100 mL) at −68° C. was added LDA (2 M in THF, 18.0 mL, 36.0 mmol, 1.50 eq), and the mixture was stirred for 1 h under N₂ atmosphere. 1-(5-methylisoxazol-3-yl)ethanone (3.00 g, 23.9 mmol, 1.00 eq) was then added to the reaction mixture at −68° C., and the reaction mixture was stirred at 25° C. for 15 h. The reaction mixture was quenched by dropwise addition sat. aq. NH₄Cl (200 mL) and extracted with ethyl acetate (100 mL×3). The combined organic layers were washed with brine (100 mL), dried over Na₂SO₄, filtered, and concentrated under reduced pressure. Purification by silica gel chromatography gave 5-methyl-3-(prop-1-en-2-yl)isoxazole (Intermediate A-25).

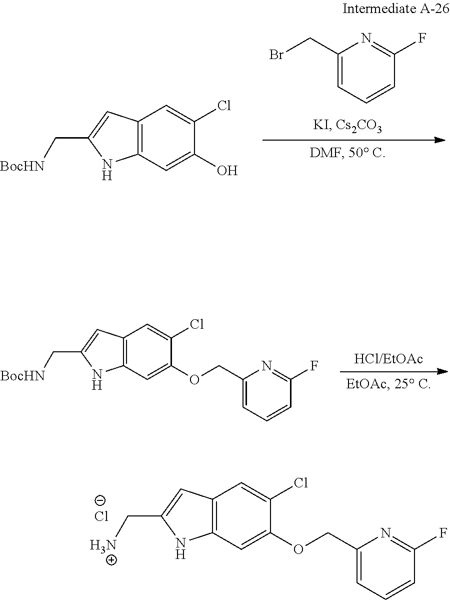

### Step 1: tert-butyl ((5-chloro-6-((6-fluoropyridin-2-yl)methoxy)-1H-indol-2-yl)methyl)carbamate

To a mixture of tert-butyl ((5-chloro-6-hydroxy-1H-indol-2-yl)methyl)carbamate (1.30 g, 4.38 mmol, 1.00 eq), 2-(bromomethyl)-6-fluoropyridine (832 mg, 4.38 mmol, 1.00 eq) in DMF (10 mL) were added KI (50.9 mg, 306 μmol, 0.07 eq) and K₂CO₃ (605 mg, 4.38 mmol, 1.00 eq) in one portion at 25° C., then the reaction mixture was heated and stirred at 50° C. for 1 h under N₂ atmosphere. After cooling to the room temperature, the reaction mixture was quenched by addition of H₂O (50 mL) and then extracted with ethyl acetate (3×30 mL). The combined organic layers were washed with brine (1×20 mL), dried over Na₂SO₄, filtered, and concentrated under reduced pressure to give a residue. The residue was purified by column chromatography to give tert-butyl ((5-chloro-6-((6-fluoropyridin-2-yl)methoxy)-1H-indol-2-yl)methyl)carbamate: LC-MS (ESI) m/z calcd for C₂₀H₂₁ClFN₃O₃: 405.13; found: 406.2 [M+H]⁺.

### Step 2: (5-chloro-6-((6-fluoropyridin-2-yl)methoxy)-1H-indol-2-yl)methanamine hydrochloride

To a solution of tert-butyl ((5-chloro-6-((6-fluoropyridin-2-yl)methoxy)-1H-indol-2-yl)methyl)carbamate (1.50 g, 3.70 mmol, 1.00 eq) in ethyl acetate (5 mL) was added HCl in ethyl acetate (4 M, 20 mL, 80 mmol) in portions at 25° C. The reaction mixture was stirred at 25° C. for 2 h. The reaction mixture was concentrated under reduced pressure to give (5-chloro-6-((6-fluoropyridin-2-yl)methoxy)-1H-indol-2-yl)methanamine hydrochloride (Intermediate A-26), which was used directly without further purification. LC-MS (ESI) m/z calcd for C₁₅H₁₃ClFN₃O: 305.07; found: 289.1 [M−NH₂]⁺.

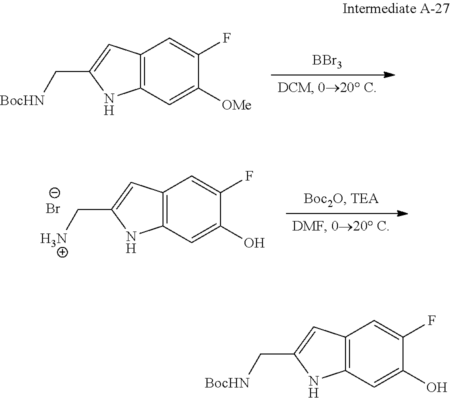

### Step 1: 2-(aminomethyl)-5-fluoro-1H-indol-6-ol hydrobromide

A 1 L three-necked flask equipped with a stir bar was charged with tert-butyl ((5-fluoro-6-methoxy-1H-indol-2-yl)methyl)carbamate (20.0 g, 71.3 mmol, 1.00 eq) and DCM (200 mL), the solution was degassed under vacuum and purged with N₂ three times. The mixture was cooled to 0° C., BBr₃ (17.1 mL, 178 mmol, 2.50 eq) was added dropwise under N₂. After the addition, the reaction mixture was allowed to warm to 20° C. and stirred for 5 h. The precipitated solids were filtered, and the filter cake was washed with DCM (4×100 mL) to remove excess BBr₃. The filter cake was collected and triturated with MTBE (200 mL) at 20° C. for 10 min. The mixture was once again filtered, and the filter cake was collected to give 2-(aminomethyl)-5-fluoro-1H-indol-6-ol hydrobromide. LC-MS (ESI) m/z calcd for C₉H₉FN₂O: 180.07; found: 164.3 [M−NH₂]⁺.

### Step 2: tert-butyl ((5-fluoro-6-hydroxy-1H-indol-2-yl)methyl)carbamate

To a solution of 2-(aminomethyl)-5-fluoro-1H-indol-6-ol hydrobromide (25.0 g, 138 mmol, 1.00 eq) in DMF (250 mL) were added TEA (70 mL, 502 mmol, 3.62 eq) and Boc₂O (25.5 mL, 111 mmol, 0.80 eq) in portions at 0° C. After the addition, the reaction was allowed to warm to 20° C. and stirred for 2 h. The mixture was quenched by addition of H₂O (200 mL) and extracted with EtOAc (3×300 ml). The combined organic layers were dried over Na₂SO₄, filtered, and concentrated under reduced pressure. Purification by column chromatography gave tert-butyl ((5-fluoro-6-hydroxy-1H-indol-2-yl)methyl)carbamate (Intermediate A-27). LC-MS (ESI) m/z calcd for C₁₄H₁₇FN₂O₃: 280.12; found: 281.1 [M+H]⁺.

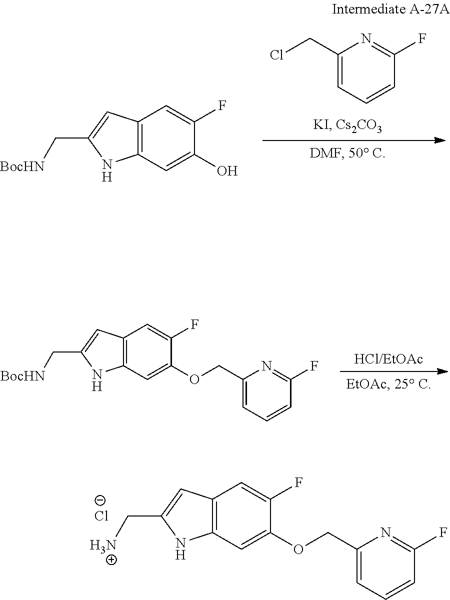

### Step 1: tert-butyl ((5-fluoro-6-((6-fluoropyridin-2-yl)methoxy)-1H-indol-2-yl)methyl)carbamate

To a mixture of tert-butyl ((5-fluoro-6-hydroxy-1H-indol-2-yl)methyl)carbamate (500 mg, 1.78 mmol, 1.00 eq) and 2-(bromomethyl)-6-fluoro-pyridine (338 mg, 1.78 mmol, 1.00 eq) in DMF (5 mL) were added KI (29.5 mg, 178 μmol, 0.10 eq) and K₂CO₃ (492 mg, 3.56 mmol, 2.00 eq) in one portion. The mixture was then purged with N₂ 3 times, then heated and stirred at 50° C. for 1 h. The reaction mixture was allowed to cool to room temperature, then was poured into water (5 mL). The aqueous phase was extracted with ethyl acetate (3×10 mL). The combined organic phase was washed with brine (2×10 mL), dried over Na₂SO₄, filtered, and concentrated under reduced pressure. Purification by column chromatography gave tert-butyl ((5-fluoro-6-((6-fluoropyridin-2-yl)methoxy)-1H-indol-2-yl)methyl)carbamate. LC-MS (ESI) m/z calcd for C₂₀H₂₁F₂N₃O₃: 389.16; found: 390.2 [M+H]⁺.

### Step 2: (5-fluoro-6-((6-fluoropyridin-2-yl)methoxy)-1H-indol-2-yl)methanamine hydrochloride

To a solution of tert-butyl ((5-fluoro-6-((6-fluoropyridin-2-yl)methoxy)-1H-indol-2-yl)methyl) carbamate (150 mg, 385 μmol, 1.00 eq) in EtOAc (0.5 mL) was added HCl/EtOAc (4 M, 2 mL, 8.00 mmol), and the reaction mixture was stirred at 25° C. for 1 h. The mixture was concentrated under reduced pressure to give (5-fluoro-6-((6-fluoropyridin-2-yl)methoxy)-1H-indol-2-yl)methanamine hydrochloride (Intermediate A-27A), which was used directly without further purification. LC-MS (ESI) m/z calcd for C₁₅H₁₃F₂N₃O: 305.07; found 289.10; found: 273.1 [M−NH₂]⁺.

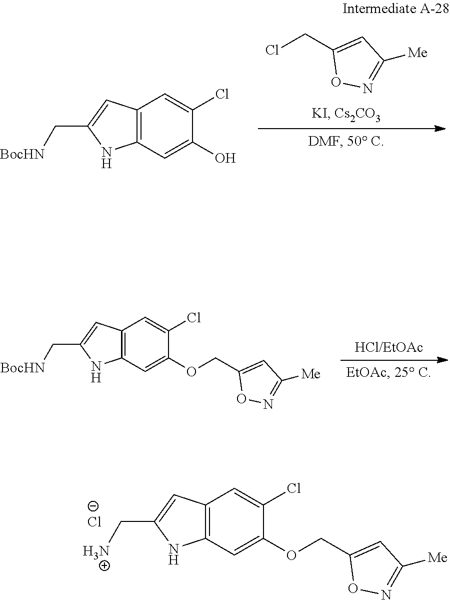

### Step 1: tert-butyl ((5-chloro-6-((3-methylisoxazol-5-yl)methoxy)-1H-indol-2-yl)methyl)carbamate

To a mixture of tert-butyl ((5-chloro-6-hydroxy-1H-indol-2-yl)methyl)carbamate (0.50 g, 1.68 mmol, 1.00 eq) and 5-(chloromethyl)-3-methylisoxazole (222 mg, 1.68 mmol, 1.00 eq) in DMF (5 mL) were added Cs₂CO₃ (1.65 g, 5.05 mmol, 3.00 eq) and KI (55.9 mg, 337 μmol, 0.20 eq) in one portion at 20° C. The reaction mixture was heated to 50° C. and stirred for 1 h. The mixture was allowed to cool to room temperature, then was quenched by addition of water (20 mL) and then extracted with ethyl acetate (3×20 mL). The combined organic phase was washed with brine (20 mL), dried over anhydrous sodium sulfate, filtered, and concentrated under reduced pressure. Purification by column chromatography gave tert-butyl ((5-chloro-6-((3-methylisoxazol-5-yl)methoxy)-1H-indol-2-yl)methyl)carbamate. LC-MS (ESI) m/z calcd for C₁₉H₂₂ClN₃O₄: 391.13: found: 392.1 [M+H]⁺.

### Step 2: (5-chloro-6-((3-methylisoxazol-5-yl)methoxy)-1H-indol-2-yl)methanamine hydrochloride

To a 0° C. solution of tert-butyl ((5-chloro-6-((3-methylisoxazol-5-yl)methoxy)-1H-indol-2-yl)methyl)carbamate (500 mg, 1.28 mmol, 1.00 eq) in EtOAc (5 mL) was added HCl/EtOAc (4 M, 5 mL, 20.0 mmol) in portions. After the addition was complete, the reaction mixture was allowed to warm to 20° C. and stir for 1 h. The reaction mixture was concentrated under reduced pressure to give (5-chloro-6-((3-methylisoxazol-5-yl)methoxy)-1H-indol-2-yl)methanamine hydrochloride (Intermediate A-28) which was used directly without further purification. LC-MS (ESI) m/z calcd for C₁₄H₁₄ClN₃O₂: 291.08: found: 275.2 [M−NH₂]⁺.

The following compounds in Table B-28 were synthesized using procedures similar to Intermediate A-28 using the appropriate starting materials.

TABLE B-28

| Intermediate | | | Exact mass | LCMS, |
|---|---|---|---|---|
| No. | Structure | IUPAC name | (g/mol) | found [M]⁺ |
| B-28-1 | 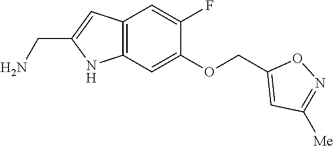 [SMILES: Cc1cc(COc2cc3[nH]c(CN)cc3cc2F)on1] | (5-fluoro-6-((3- methylisoxazol- 5-yl)methoxy)- 1H-indol-2- yl)methanamine | 275.11 | 259.2 [M − NH₂]⁺ |

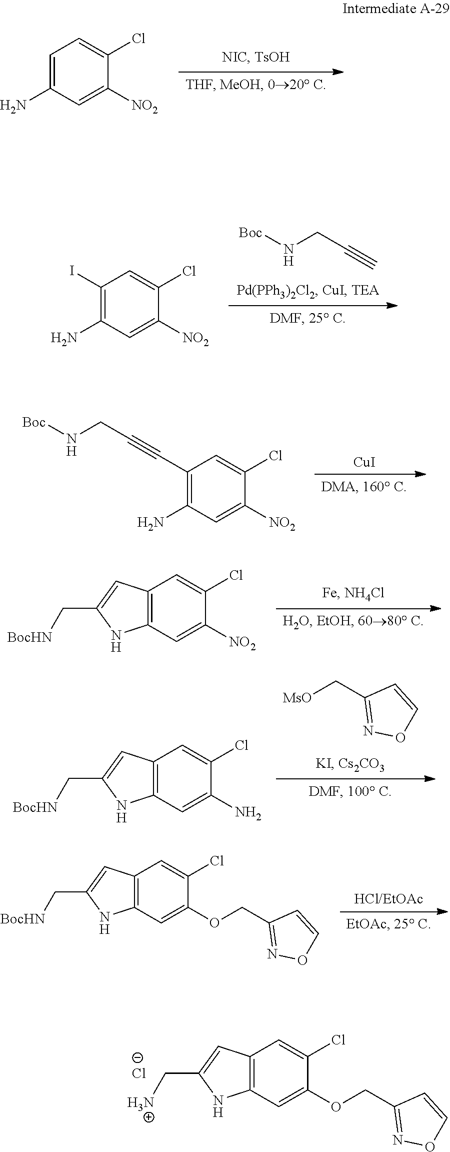

### Step 1: 4-chloro-2-iodo-5-nitroaniline

To a 0° C. solution of 4-chloro-3-nitroaniline (12.5 g, 72.4 mmol, 1.00 eq) in THF (130 mL) and MeOH (130 mL) were added NIS (13.0 g, 57.9 mmol, 0.80 eq) and PTSA monohydrate (13.8 g, 72.4 mmol, 1.00 eq) in portions. After the addition, the reaction mixture was allowed to warm to 25° C. and stir for 1 h. The mixture was poured into saturated aq. Na₂S₂O₃ (100 mL) and stirred for 3 min. The aqueous phase was then extracted with ethyl acetate (3×100 mL). The combined organic layers were washed with brine (3×100 mL), dried over anhydrous Na₂SO₄, filtered, and concentrated under reduced pressure. Purification by column chromatography gave 4-chloro-2-iodo-5-nitroaniline. LC-MS (ESI) m/z calcd for C₆H₄ClIN₂O₂: 297.90: found: 298.9 [M+H]⁺.

### Step 2: tert-butyl (3-(2-amino-5-chloro-4-nitrophenyl)prop-2-yn-1-yl)carbamate

To a mixture of tert-butyl prop-2-yn-1-ylcarbamate (5.74 g, 36.9 mmol, 1.20 eq) and 4-chloro-2-iodo-5-nitro-aniline (9.20 g, 30.8 mmol, 1.00 eq) in DMF (100 mL) was added CuI (1.17 g, 6.16 mmol, 0.20 eq) in one portion at 25° C. The system was degassed and then charged with nitrogen three times. Pd(PPh₃)₂Cl₂ (2.16 g, 3.08 mmol, 0.10 eq), TEA (154 mmol, 21.4 mL, 5.00 eq) were then added under N₂. The reaction mixture was stirred at 25° C. for 16 h. The mixture was poured into water (50 mL) and stirred for 3 min. The aqueous phase was then extracted with ethyl acetate (3×50 mL). The combined organic layers were washed with brine (3×50 mL), dried over anhydrous Na₂SO₄, filtered, and concentrated under reduced pressure. Purification by column chromatography gave tert-butyl (3-(2-amino-5-chloro-4-nitrophenyl)prop-2-yn-1-yl)carbamate. LC-MS (ESI) m/z calcd for C₁₄H₁₆ClN₃O₄: 325.08; found: 326.2 [M+H]⁺.

### Step 3: tert-butyl ((5-chloro-6-nitro-1H-indol-2-yl)methyl)carbamate

To a solution of tert-butyl (3-(2-amino-5-chloro-4-nitrophenyl)prop-2-yn-1-yl)carbamate (6.40 g, 19.6 mmol, 1.00 eq) in DMA (70 mL) was added CuI (1.87 g, 9.82 mmol, 0.50 eq) in one portion at 25° C. The reaction mixture was heated to 160° C. and stirred for 1 h. The mixture was allowed to cool to room temperature, then was poured into water (70 mL) and stirred for 3 min. The aqueous phase was extracted with DCM (3×100 mL). The combined organic layers were washed with brine (300 mL), dried over anhydrous Na₂SO₄, filtered, and concentrated under reduced pressure. Purification by column chromatography gave tert-butyl ((5-chloro-6-nitro-1H-indol-2-yl)methyl)carbamate. LC-MS (ESI) m/z calcd for C₁₄H₁₆ClN₃O₄: 325.08; found: 326.2 [M+H]⁺.

### Step 4: tert-butyl ((6-amino-5-chloro-1H-indol-2-yl)methyl)carbamate

To a solution of tert-butyl ((5-chloro-6-nitro-1H-indol-2-yl)methyl)carbamate (3.80 g, 11.6 mmol, 1.00 eq) in H₂O (10 mL) and EtOH (50 mL) was added NH₄Cl (2.50 g, 46.6 mmol, 4.00 eq) in one portion at 25° C. The reaction mixture was then heated to 60° C., and Fe (6.51 g, 116 mmol, 10.0 eq) was added portionwise. After the addition was complete, the reaction mixture was heated to 80° C. and stirred for 1 h. The mixture was allowed cool to room temperature, then was filtered through a pad of Celite. The filtrate was concentrated in vacuo, diluted with H₂O (30 mL), and extracted with ethyl acetate (2×40 mL). The combined organic phase was washed with brine (2×30 mL), dried over anhydrous Na₂SO₄, filtered, and concentrated under reduced pressure. Purification by column chromatography gave tert-butyl ((6-amino-5-chloro-1H-indol-2-yl)methyl)carbamate. LC-MS (ESI) m/z calcd for C₁₄H₁₈ClN₃O₂: 295.11; found: 296.1 [M+H]⁺.

### Step 5: tert-butyl ((5-chloro-6-((isoxazol-3-ylmethyl)amino)-1H-indol-2-yl)methyl)carbamate

To a mixture of tert-butyl ((6-amino-5-chloro-1H-indol-2-yl)methyl)carbamate (500 mg, 1.69 mmol, 1.00 eq) and isoxazol-3-ylmethyl methanesulfonate (329 mg, 1.86 mmol, 1.10 eq) in DMF (5 mL) were added KI (28.0 mg, 169 μmol, 0.10 eq) and K₂CO₃ (584 mg, 4.23 mmol, 2.50 eq). The system was degassed and charged with nitrogen three times. The reaction mixture was then heated to 100° C. and stirred for 3 h. The reaction mixture was allowed to cool to 20° C., then was quenched by addition of water (15 mL) and extracted with ethyl acetate (3×20 mL). The combined organic phase was washed with brine (20 mL), dried over anhydrous sodium sulfate, filtered, and concentrated under reduced pressure. Purification by column chromatography gave tert-butyl ((5-chloro-6-((isoxazol-3-ylmethyl)amino)-1H-indol-2-yl)methyl)carbamate. LC-MS (ESI) m/z calcd for C₁₈H₂₁ClN₄O₃: 376.13; found: 377.1 [M+H]⁺.

### Step 6: 2-(aminomethyl)-5-chloro-N-(isoxazol-3-ylmethyl)-1H-indol-6-amine hydrochloride

To a mixture of tert-butyl ((5-chloro-6-((isoxazol-3-ylmethyl)amino)-1H-indol-2-yl)methyl)carbamate (600 mg, 1.59 mmol, 1.00 eq) in EtOAc (1 mL) was added HCl in EtOAc (4 M, 5 mL, 20.0 mmol) portionwise. The reaction mixture was stirred at 25° C. for 2 h, then was concentrated under reduced pressure to give 2-(aminomethyl)-5-chloro-N-(isoxazol-3-ylmethyl)-1H-indol-6-amine hydrochloride (Intermediate A-29), which was used directly without further purification. LC-MS (ESI) m/z calcd for C₁₃H₁₃ClN₄O: 276.08; found: 260.1 [M−NH₂]⁺.

The following compounds in Table B-29 were synthesized using procedures similar to Intermediate A-29 using the appropriate starting materials.

TABLE B-29

| Intermediate | | | Exact mass | LCMS, |
|---|---|---|---|---|
| No. | Structure | IUPAC name | (g/mol) | found [M]⁺ |
| B-29-1 | 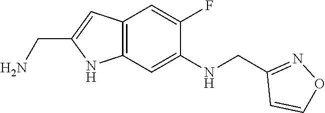 [SMILES: NCc1cc2cc(F)c(NCc3ccon3)cc2[nH]1] | 2-(aminomethyl)-5- fluoro-N-(isoxazol- 3-ylmethyl)-1H- indol-6-amine | 260.11 | 244.2 [M − NH₂]⁺ |
| B-29-2 | 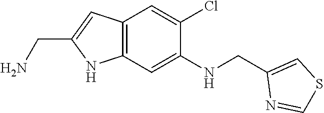 [SMILES: NCc1cc2cc(Cl)c(NCc3cscn3)cc2[nH]1] | 2-(aminomethyl)-5- chloro-N-(thiazol-4- ylmethyl)-1H-indol- 6-amine | 292.05 | 293.2 |
| B-29-3 | 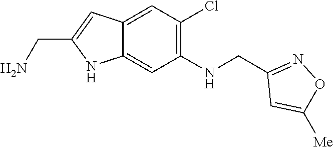 [SMILES: Cc1cc(CNc2cc3[nH]c(CN)cc3cc2Cl)no1] | 2-(aminomethyl)-5- chloro-N-((5- methylisoxazol-3- yl)methyl)-1H- indol-6-amine | 290.09 | 291.0 |
| B-29-4 | 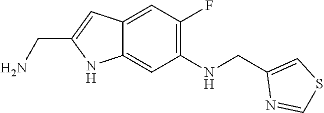 [SMILES: NCc1cc2cc(F)c(NCc3cscn3)cc2[nH]1] | 2-(aminomethyl)-5- fluoro-N-(thiazol-4- ylmethyl)-1H-indol- 6-amine | 276.08 | 277.1 |

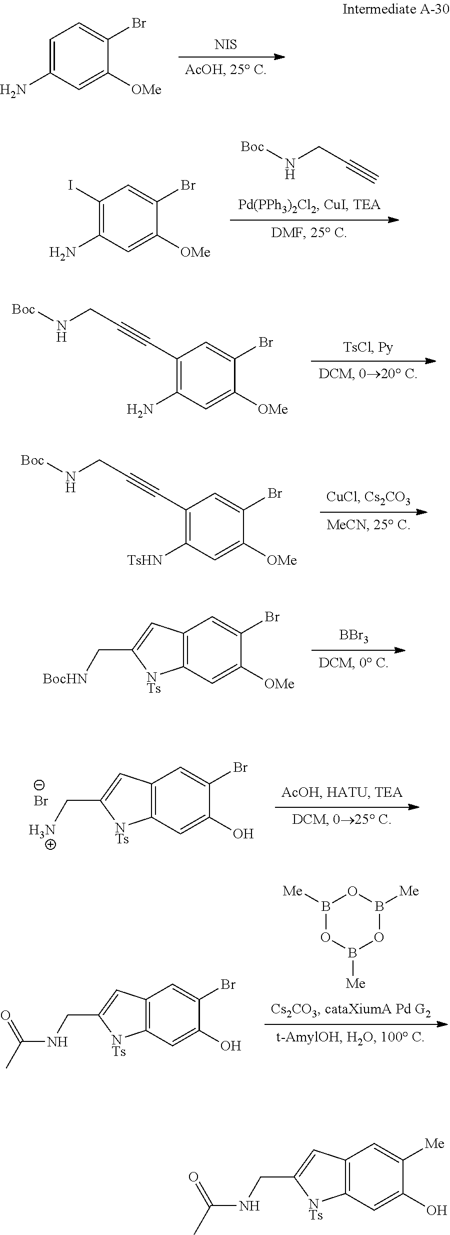

### Step 1: 4-bromo-2-iodo-5-methoxyaniline

Two reactions were carried out in parallel. To a solution of 4-bromo-3-methoxyaniline (100 g, 494 mmol, 1.00 eq) in AcOH (500 mL) was added NIS (111 g, 494 mmol, 1.00 eq) in portions at 25° C., then the reaction mixture was for 2 h, whereupon the two reaction mixtures were combined. H₂O (1 L) was added dropwise to the combined reaction mixture at 25° C. The mixture was then cooled to −5° C. and stirred for 1 h. The resulting solid was filtered, and the filter cake was dried to give 4-bromo-2-iodo-5-methoxyaniline, which was used directly without further purification. LC-MS (ESI) m/z calcd for C₇H₇BrINO: 326.88; found: 327.9 [M+H]⁺.

### Step 2: tert-butyl (3-(2-amino-5-bromo-4-methoxyphenyl)prop-2-yn-1-yl)carbamate

Three reactions were carried out in parallel. To a solution of 4-bromo-2-iodo-5-methoxyaniline (102 g, 311 mmol, 1.00 eq) in DMF (300 mL) were added tert-butyl N-prop-2-ynylcarbamate (48.2 g, 311 mmol, 1.00 eq) and TEA (1.56 mol, 216 mL, 5.00 eq). The resulting mixture was degassed with N₂ three times. Pd(PPh₃)₂Cl₂ (4.37 g, 6.22 mmol, 0.02 eq) and CuI (5.92 g, 31.1 mmol, 0.1 eq) were then added under N₂, and the reaction mixture was stirred at 25° C. for 12 h. The three reaction mixtures were then combined, poured into water (1.5 L), and stirred for 10 min. The aqueous phase was extracted with ethyl acetate (3×500 mL). The combined organic phases were washed with brine (3×500 mL), dried over anhydrous Na₂SO₄, filtered, and concentrated under reduced pressure. The residue was dissolved with dichloromethane (200 mL) and, with rapid stirring, hexane (2000 mL) was added dropwise. After the addition was complete, the mixture was stirred at 25° C. for 1 h. The resulting solids were filtered and the filter cake was collected to give tert-butyl (3-(2-amino-5-bromo-4-methoxyphenyl)prop-2-yn-1-yl)carbamate, which was used without further purification. LC-MS (ESI) m/z calcd for C₁₅H₁₉BrN₂O₃: 354.06; found: 299.2 [M−t-Bu+H]⁺.

### Step 3: tert-butyl (3-(5-bromo-4-methoxy-2-((4-methylphenyl) sulfonamido) phenyl) prop-2-yn-1-yl) carbamate

Two reactions were carried out in parallel. To a 0° C. mixture of tert-butyl (3-(2-amino-5-bromo-4-methoxyphenyl)prop-2-yn-1-yl)carbamate (100 g, 281 mmol, 1.00 eq) and pyridine (563 mmol, 45.4 mL, 2.00 eq) in dichloromethane (500 mL) was added TsCl (59.0 g, 309 mmol, 1.10 eq) dropwise. The reaction mixture was allowed to warm to 20° C. and stir for 12 h. The two reaction mixtures were then combined, and the resulting mixture was quenched by addition of H₂O (1 L) and extracted with dichloromethane (3×1 L). The combined organic layers were washed with brine (2×500 mL), dried over Na₂SO₄, filtered, and concentrated under reduced pressure. The residue was triturated with MTBE (200 mL) at 25° C. for 30 min, then filtered, and the filter cake was collected to give tert-butyl (3-(5-bromo-4-methoxy-2-((4-methylphenyl) sulfonamido) phenyl) prop-2-yn-1-yl) carbamate. LC-MS (ESI) m/z calcd for C22H₂₅BrN₂O₅: 508.07; found: 453.0 [M−tBu+H]⁺.

### Step 4: tert-butyl ((5-bromo-6-methoxy-1-tosyl-1H-indol-2-yl)methyl)carbamate

Two reactions were carried out in parallel. To a solution of give tert-butyl (3-(5-bromo-4-methoxy-2-((4-methylphenyl) sulfonamido) phenyl) prop-2-yn-1-yl) carbamate (100 g, 196 mmol, 1.00 eq) in MeCN (500 mL) were added CuCl (2.92 g, 29.4 mmol, 0.15 eq) and Cs₂CO₃ (9.59 g, 29.4 mmol, 0.15 eq), and the reaction mixture was stirred at 25° C. for 12 h. The 2 reaction mixtures were combined, poured into ice water (1 L), and stirred for 10 min. The aqueous phase was extracted with ethyl acetate (3×1 L). The combined organic phase was washed with brine (2×1 L), dried with anhydrous Na₂SO₄, filtered, and concentrated under reduced pressure. The crude product was triturated with methyl tert-butyl ether (200 mL) at 20° C. for 30 min and the mixture was filtered. The filter cake was collected and dried under reduced pressure to give tert-butyl ((5-bromo-6-methoxy-1-tosyl-1H-indol-2-yl)methyl) carbamate. LC-MS (ESI) m/z calcd for C₂₂H₂₅BrN₂O₅S: 508.07; found: 453.0 [M−tBu+H]⁺.

### Step 5: 2-(aminomethyl)-5-bromo-1-tosyl-1H-indol-6-ol hydrobromide

To a 0° C. solution of tert-butyl ((5-bromo-6-methoxy-1-tosyl-1H-indol-2-yl)methyl)carbamate (10.0 g, 19.6 mmol, 1.00 eq) in dichloromethane (120 mL) was added BBr₃ (78.5 mmol, 7.57 mL, 4.00 eq) dropwise under N₂. Then the reaction mixture was stirred at 0° C. for 1 h. The reaction mixture was added to MeOH (80 mL) dropwise at 0° C., and the mixture was allowed to warm to 20° C. and stir for 1 h. The mixture was then concentrated under reduced pressure. The crude product was triturated with methyl tert-butyl ether (30 mL) at 25° C. for 10 min, then was filtered. The filter cake was washed with MTBE (10 mL) and dried under reduced pressure to give 2-(aminomethyl)-5-bromo-1-tosyl-1H-indol-6-ol hydrobromide. LC-MS (ESI) m/z calcd for C₁₆H₁₅BrN₂O₃S: 394.00; found: 378.0 [M−NH₂]⁺.

### Step 6: N-((5-bromo-6-hydroxy-1-tosyl-1H-indol-2-yl)methyl)acetamide

To a 0° C. solution of AcOH (126 mg, 2.10 mmol, 1.0 eq) in DMF (10 mL) were added TEA (637 mg, 6.30 mmol, 3.00 eq) and HATU (878 mg, 2.31 mmol, 1.10 eq) in portions under N₂. The reaction mixture was stirred at 0° C. for 0.5 h. To the reaction mixture was then added 2-(aminomethyl)-5-bromo-1-tosyl-1H-indol-6-ol hydrobromide (1.00 g, 2.10 mmol, 1.00 eq) portionwise at 0° C. After the addition was complete, the reaction mixture was allowed to warm to 25° C. and stir for 1.5 h. The reaction mixture was poured into water (30 mL) and stirred for 5 min. The aqueous phase was extracted with ethyl acetate (3×30 mL). The combined organic phase was washed with brine (2×20 mL), dried over anhydrous Na₂SO₄, filtered, and concentrated under reduced pressure. Purification by column chromatography gave N-((5-bromo-6-hydroxy-1-tosyl-1H-indol-2-yl)methyl)acetamide. LC-MS (ESI) m/z calcd for C₁₈H₁₇BrN₂O₄S: 436.01; found: 437.1 [M+H]⁺.

### Step 7: N-((6-hydroxy-5-methyl-1-tosyl-1H-indol-2-yl)methyl)acetamide

Two reactions were carried out in parallel. To a solution of N-((5-bromo-6-hydroxy-1-tosyl-1H-indol-2-yl)methyl)acetamide (1.00 g, 2.29 mmol, 1.00 eq) and 2,4,6-trimethyl-1,3,5,2,4,6-trioxatriborinane (1.44 g, 11.4 mmol, 5.00 eq) in 2-methyl-2-butanol (5 mL) and H₂O (1 mL) were added Cs₂CO₃ (1.49 g, 4.57 mmol, 2.00 eq) and chloro[(di(1-adamantyl)-N-butylphosphine)-2-(2-aminobiphenyl)]palladium(II) (305 mg, 457 μmol, 0.20 eq) under N₂. The resulting mixture was degassed and charged with nitrogen three times, then was heated to 100° C. and stirred for 12 h. The two reaction mixtures were then combined and allowed to cool to room temperature. The combined reaction mixture was poured into water (30 mL) and stirred for 5 min. The aqueous phase was extracted with ethyl acetate (3×20 mL). The combined organic phases were washed with brine (2×20 mL), dried over anhydrous Na₂SO₄, filtered, and concentrated under reduced pressure. The crude product was triturated with methyl tert-butyl ether (3 mL) at 25° C. for 10 min, then filtered. The filter cake was washed with MTBE (10 mL) and dried in vacuo to give N-((6-hydroxy-5-methyl-1-tosyl-1H-indol-2-yl)methyl)acetamide (Intermediate A-30). LC-MS (ESI) m/z calcd for C₁₉H₂₀N₂O₄S: 372.11; found: 373.2 [M+H]⁺.

The following compounds in Table B-30 were synthesized using procedures similar to Intermediate A-30 using the appropriate starting materials.

TABLE B-30

| Intermediate | | | Exact mass | LCMS, |
|---|---|---|---|---|
| No. | Structure | IUPAC name | (g/mol) | found [M]⁺ |
| B-30-1 | 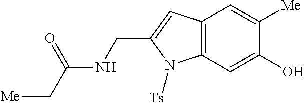 [SMILES: CCC(=O)NCc1cc2cc(C)c(O)cc2n1S(=O)(=O)c1ccc(C)cc1] | N-((6-hydroxy-5- methyl-1-tosyl-1H- indol-2- yl)methyl)propionamide | 386.13 | 387.2 |

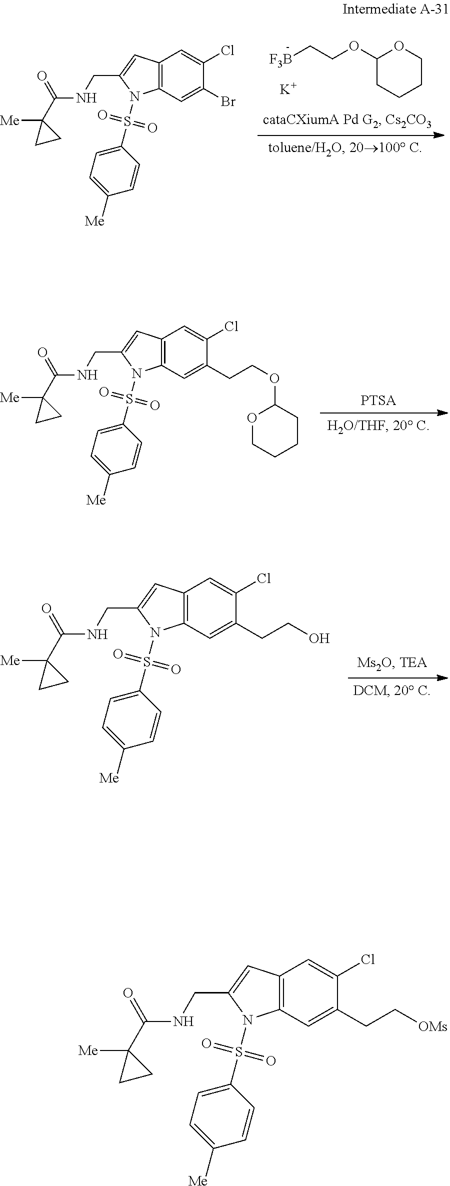

### Step 1: N-((5-chloro-6-(2-((tetrahydro-2H-pyran-2-yl)oxy)ethyl)-1-tosyl-1H-indol-2-yl)methyl)-1-methylcyclopropane-1-carboxamide

To a mixture of N-((6-bromo-5-chloro-1-tosyl-1H-indol-2-yl)methyl)-1-methylcyclopropane-1-carboxamide (1.00 g, 2.02 mmol, 1.00 eq) and potassium trifluoro(2-((tetrahydro-2H-pyran-2-yl)oxy)ethyl)borate (571 mg, 2.42 mmol, 1.20 eq) in H₂O (1 mL) and toluene (10 mL) was added Cs₂CO₃ (1.31 g, 4.03 mmol, 2.00 eq) and [2-(2-aminophenyl)phenyl]-chloro-palladium;bis(1-adamantyl)-butyl-phosphane (134 mg, 201 μmol, 0.10 eq) in one portion at 20° C. under N₂. The reaction system was degassed and then charged with nitrogen three times. The reaction mixture was heated to 100° C. and stirred at 100° C. for 16 h. After cooling to room temperature, the reaction mixture was quenched by addition of H₂O (30 mL) at 20° C., then the aqueous phase was extracted with ethyl acetate (3×10 mL). The combined organic layers were washed with brine (30 mL), dried over Na₂SO₄, filtered, and concentrated under reduced pressure to give a residue. The residue was purified by column chromatography to give N-((5-chloro-1-(phenylsulfonyl)-6-(2-((tetrahydro-2H-pyran-2-yl)oxy)ethyl)-1H-indol-2-yl)methyl)-1-methylcyclopropane-1-carboxamide. LC-MS (ESI) m/z calcd for C₂₈H₃₃ClN₂O₅S: 544.18; found 545.4 [M+H]⁺.

### Step 2: N-((5-chloro-6-(2-hydroxyethyl)-1-tosyl-1H-indol-2-yl)methyl)-1-methylcyclopropane-1-carboxamide

To a mixture of N-((5-chloro-6-(2-((tetrahydro-2H-pyran-2-yl)oxy)ethyl)-1-tosyl-1H-indol-2-yl)methyl)-1-methylcyclopropane-1-carboxamide (970 mg, 1.78 mmol, 1.00 eq) in THF (10 mL) and H₂O (1 mL) was added 4-methylbenzenesulfonic acid (PTSA, 1.53 g, 8.90 mmol, 5.00 eq) in one portion at 20° C. under N₂. The reaction mixture was stirred at 20° C. for 5 h. The reaction mixture was quenched by addition of H₂O (10 mL) at 20° C., then extracted with ethyl acetate (3×10 mL). The combined organic layers were washed with brine (20 mL), dried over Na₂SO₄, filtered, and concentrated under reduced pressure to give N-((5-chloro-6-(2-hydroxyethyl)-1-tosyl-1H-indol-2-yl)methyl)-1-methylcyclopropane-1-carboxamide, which was used for next step directly without of further purification. LC-MS (ESI) m/z calcd for C₂₃H₂₅ClN₂O₄S: 460.12; found 461.2 [M+H]⁺.

### Step 3: 2-(5-chloro-2-((1-methylcyclopropane-1-carboxamido)methyl)-1-tosyl-1H-indol-6-yl)ethyl methanesulfonate

To a mixture of N-((5-chloro-6-(2-hydroxyethyl)-1-tosyl-1H-indol-2-yl)methyl)-1-methylcyclopropane-1-carboxamide (460 mg, 997 μmol, 1.00 eq) in DCM (10 mL) was added TEA (302 mg, 2.99 mmol, 3.00 eq) and methylsulfonyl methanesulfonate (Ms₂O, 260 mg, 1.50 mmol, 1.50 eq) in one portion at 20° C. under N₂. The reaction mixture was stirred at 20° C. for 30 mins. The reaction mixture was quenched by addition of H₂O (10 mL) at 20° C., then extracted with ethyl acetate (3×10 mL). The combined organic layers were washed with brine (20 mL), dried over Na₂SO₄, filtered, and concentrated under reduced pressure to give 2-(5-chloro-2-((1-methylcyclopropane-1-carboxamido)methyl)-1-tosyl-1H-indol-6-yl)ethyl methanesulfonate, which was used for next step directly without of further purification. LC-MS (ESI) m/z calcd for C₂₄H₂₇ClN₂O₆S₂: 538.10; found 539.2 [M+H]⁺.

The following compounds in Table B-31 were synthesized using procedures similar to Intermediate A-31 using the appropriate starting materials.

TABLE B-31

| | | | Exact | LCMS, |
|---|---|---|---|---|
| Intermediate | | | mass | found |
| No. | Structure | IUPAC name | (g/mol) | [M]⁺ |
| B-31-1 | 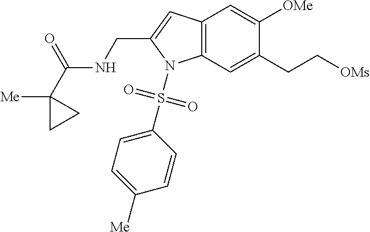 [SMILES: COc1cc2cc(CNC(=O)C3(C)CC3)n(S(=O)(=O)c3ccc(C)cc3)c2cc1CCOS(C)(=O)=O] | 2-(5-methoxy-2-((1- methylcyclopropane-1- carboxamido)methyl)- 1-tosyl-1H-indol-6- yl)ethyl methanesulfonate | 534.15 | 535.2 |

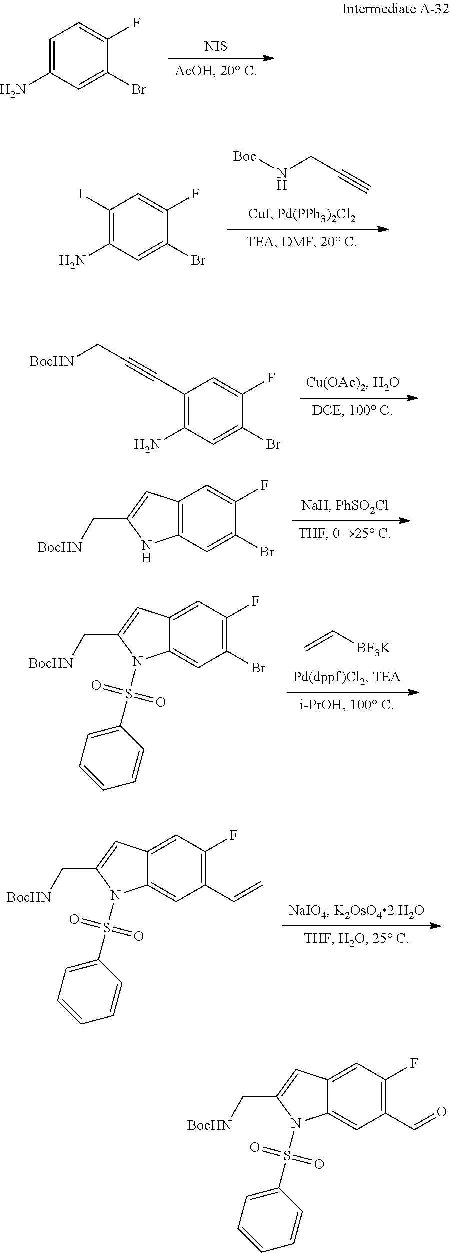

### Step 1: 5-bromo-4-fluoro-2-iodoaniline

To a solution of 3-bromo-4-fluoroaniline (45.0 g, 237 mmol, 1.00 eq) in AcOH (400 mL) was added NIS (53.3 g, 237 mmol, 1.00 eq) in one portion at 20° C. under N₂. The reaction mixture was stirred at 20° C. for 1 h. The reaction mixture was quenched with H₂O (500 mL), then extracted with EtOAc (3×100 ml). The combined organic layers were washed with water (3×100 mL), dried over Na₂SO₄, filtered, and concentrated under reduced pressure to give a residue. The residue was triturated with Petroleum ether at 20° C. for 15 min. Then filtered and the filter cake was collected to give 5-bromo-4-fluoro-2-iodoaniline. LC-MS (ESI) m/z calcd for C₆H₄BrFIN: 314.86; found: 315.9 [M+H]⁺.

### Step 2: tert-butyl (3-(2-amino-4-bromo-5-fluorophenyl)prop-2-yn-1-yl)carbamate

To a mixture of 5-bromo-4-fluoro-2-iodoaniline (29.0 g, 91.8 mmol, 1.00 eq) and tert-butyl N-prop-2-ynylcarbamate (15.7 g, 101 mmol, 1.10 eq) in DMF (250 mL) were added TEA (46.5 g, 459 mmol, 63.9 mL, 5.00 eq), CuI (1.75 g, 9.18 mmol, 0.10 eq) and Pd(PPh₃)₂Cl₂ (1.93 g, 2.75 mmol, 0.03 eq) in one portion at 20° C. under N₂. The reaction mixture was stirred at 20° C. for 6 h. The reaction mixture was quenched with H₂O (500 mL), extracted with EtOAc (3×100 mL). The combined organic layers were washed with water (3×100 mL), dried over Na₂SO₄, filtered, and concentrated under reduced pressure to give a residue. The residue was purified by column chromatography to give tert-butyl (3-(2-amino-4-bromo-5-fluorophenyl)prop-2-yn-1-yl)carbamate. LC-MS (ESI) m/z calcd for C₁₄H₁₆BrFN₂O₂: 342.04; found: 286.9 [M−tBu+H]⁺.

### Step 3: tert-butyl ((6-bromo-5-fluoro-1H-indol-2-yl)methyl)carbamate

To a solution of tert-butyl (3-(2-amino-4-bromo-5-fluorophenyl)prop-2-yn-1-yl)carbamate (23.3 g, 67.9 mmol, 1.00 eq) in 1,2-dichloroethane (260 mL) was added Cu(OAc)₂·H₂O (6.78 g, 34.0 mmol, 6.78 mL, 0.50 eq) in one portion at 20° C. under N₂. The reaction mixture was heated to 100° C. for 3 h. After cooling to room temperature, the reaction mixture was quenched with H₂O (100 mL), extracted with EtOAc (3×150 mL). The combined organic layers were washed with water (3×150 mL), dried over Na₂SO₄, filtered and concentrated under reduced pressure to give a residue. The residue was purified by column chromatography to give tert-butyl ((6-bromo-5-fluoro-1H-indol-2-yl)methyl)carbamate. LC-MS (ESI) m/z calcd for C₁₄H₁₆BrFN₂O₂: 342.04; found: 286.9 [M−tBu+H]⁺.

### Step 4: tert-butyl ((6-bromo-5-fluoro-1-(phenylsulfonyl)-1H-indol-2-yl)methyl)carbamate

To a solution of NaH (2.27 g, 56.8 mmol, 60% purity, 2.50 eq) in THF (100 mL) was added a solution of tert-butyl ((6-bromo-5-fluoro-1H-indol-2-yl)methyl)carbamate (7.80 g, 22.7 mmol, 1.00 eq) in THF (100 mL) at 0° C. under N₂ atmosphere. The reaction mixture was stirred at 0° C. for 1 h. Then benzenesulfonyl chloride (5.22 g, 29.5 mmol, 1.30 eq) was added dropwise at 0° C. After the addition, the reaction mixture was warmed to 25° C. for 2 h. The reaction mixture was quenched by addition of sat. aq. NH₄Cl solution (50 mL) at 0° C., then extracted with EtOAc (3×50 mL). The combined organic layers were washed with brine (100 mL), dried over Na₂SO₄, filtered, and concentrated under reduced pressure to give a residue. The residue was purified by column chromatography to give tert-butyl ((6-bromo-5-fluoro-1-(phenylsulfonyl)-1H-indol-2-yl)methyl)carbamate. LC-MS (ESI) m/z calcd for C₂₀H₂₀BrFN₂O₄S: 482.03; found: 383.0 [M−Boc+H]⁺.

### Step 5: tert-butyl ((5-fluoro-1-(phenylsulfonyl)-6-vinyl-1H-indol-2-yl)methyl)carbamate

A mixture of tert-butyl ((6-bromo-5-fluoro-1-(phenylsulfonyl)-1H-indol-2-yl)methyl)carbamate (4.00 g, 8.28 mmol, 1.00 eq), trifluoro(vinyl)-λ⁴-borane, potassium salt (1.66 g, 12.4 mmol, 1.50 eq) and TEA (2.51 g, 24.8 mmol, 3.00 eq) in i-PrOH (40 mL) was degassed and purged with N₂ three times, and then Pd(dppf)Cl₂ (675 mg, 827 μmol, 0.10 eq) was added under N₂. The reaction mixture was then heated and stirred at 100° C. for 4 h under N₂ atmosphere. After cooling to room temperature, the reaction mixture was quenched by addition of H₂O (50 mL), extracted with EtOAc (3×50 mL). The combined organic layers were washed with brine (50 mL), dried over Na₂SO₄, filtered, and concentrated under reduced pressure to give a residue. The residue was purified by column chromatography to give tert-butyl ((5-fluoro-1-(phenylsulfonyl)-6-vinyl-1H-indol-2-yl)methyl)carbamate. LC-MS (ESI) m/z calcd for C₂₂H₂₃FN₂O₄S: 430.14; found: 453.2 [M+Na]⁺.

### Step 6: tert-butyl ((5-fluoro-6-formyl-1-(phenylsulfonyl)-1H-indol-2-yl)methyl)carbamate

To a solution of tert-butyl ((5-fluoro-1-(phenylsulfonyl)-6-vinyl-1H-indol-2-yl)methyl)carbamate (4.00 g, 9.29 mmol, 1.00 eq) in THF (50 mL) and H₂O (10 mL) were added NaIO₄ (7.95 g, 37.1 mmol, 4.00 eq) and potassium osmate dihydrate (342 mg, 929 μmol, 0.10 eq) in one portion. The reaction mixture was stirred at 25° C. for 0.5 h. The reaction mixture was quenched by addition of H₂O (50 mL), then extracted with EtOAc (3×50 mL). The combined organic layers were washed with brine (50 mL), dried over Na₂SO₄, filtered, and concentrated under reduced pressure to give a residue. The residue was purified by column chromatography to give tert-butyl ((5-fluoro-6-formyl-1-(phenylsulfonyl)-1H-indol-2-yl)methyl)carbamate. LC-MS (ESI) m/z calcd for C₂₁H₂₁FN₂O₅S: 432.12; found: 496.2 [M+Na+MeCN]⁺.

The following compounds in Table B-32 were synthesized using procedures similar to Intermediate A-32 using the appropriate starting materials.

TABLE B-32

| Intermediate | | | Exact mass | LCMS, |
|---|---|---|---|---|
| No. | Structure | IUPAC name | (g/mol) | found [M]⁺ |
| B-32-1 | 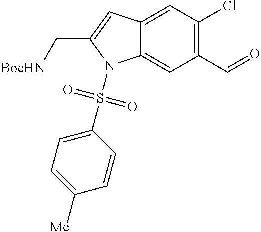 [SMILES: Cc1ccc(S(=O)(=O)n2c(CNC(=O)OC(C)(C)C)cc3cc(Cl)c(C=O)cc32)cc1] | tert-butyl ((5- chloro-6-formyl-1- tosyl-1H-indol-2- yl)methyl)carbamate | 462.10 | 407.0 [M − tBu + Na]⁺ |
| B-32-1 | 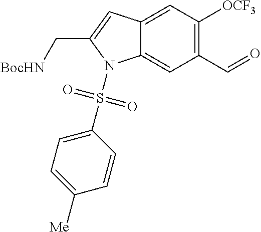 [SMILES: Cc1ccc(S(=O)(=O)n2c(CNC(=O)OC(C)(C)C)cc3cc(OC(F)(F)F)c(C=O)cc32)cc1] | tert-butyl ((6- formyl-1-tosyl-5- (trifluoromethoxy)- 1H-indol-2- yl)methyl)carbamate | 512.12 | 457.1 [M − tBu + Na]⁺ |

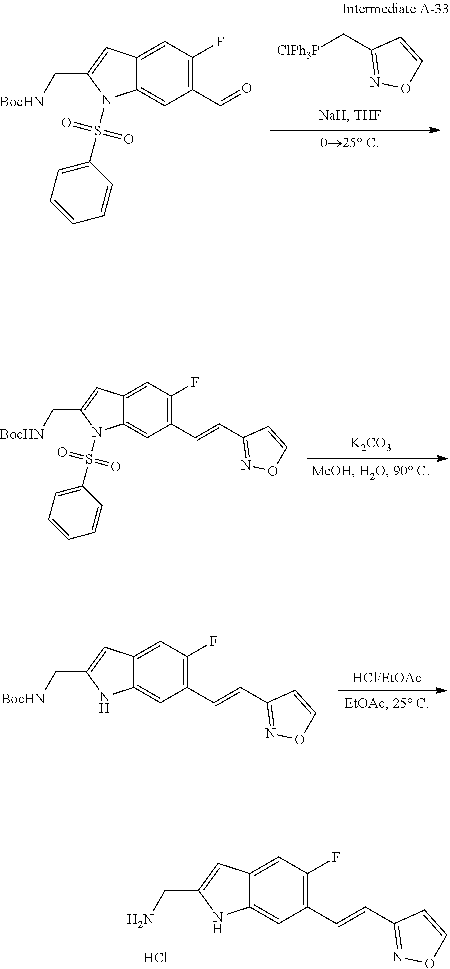

### Step 1: tert-butyl (E)-((5-fluoro-6-(2-(isoxazol-3-yl)vinyl)-1-(phenylsulfonyl)-1H-indol-2-yl)methyl)carbamate

To a solution of NaH (240 mg, 6.01 mmol, 60.0% purity, 1.30 eq) in THF (2 mL) was degassed and purged with N₂ for 3 times and cooled to 0° C., then chloro-(isoxazol-3-ylmethyl)-triphenyl-phosphane (1.76 g, 4.62 mmol, 1.00 eq) in THF (15 mL) was added dropwise at 0° C. under N₂ atmosphere. After the addition, the reaction mixture was stirred at 0° C. for 1 h. Then tert-butyl ((5-fluoro-6-formyl-1-(phenylsulfonyl)-1H-indol-2-yl)methyl)carbamate (2.00 g, 4.62 mmol, 1.00 eq) in THF (18 mL) was added dropwise at 0° C. After the addition, the reaction mixture was warmed and stirred at 20° C. for 3 h. The reaction mixture was quenched by sat. aq. NH₄Cl solution (15 mL) and extracted with EtOAc (3×20 mL). The combined organic layers were washed with brine (15 mL), dried over Na₂SO₄, filtered and concentrated under reduced pressure to give a residue. The residue was purified by column chromatography to give tert-butyl (E)-((5-fluoro-6-(2-(isoxazol-3-yl)vinyl)-1-(phenylsulfonyl)-1H-indol-2-yl)methyl)carbamate. LC-MS (ESI) m/z calcd for C₂₅H₂₄FN₃O₅S: 497.14; found: 498.2 [M+H]⁺.

### Step 2: tert-butyl (E)-((5-fluoro-6-(2-(isoxazol-3-yl)vinyl)-1H-indol-2-yl)methyl)carbamate

To a solution of tert-butyl (E)-((5-fluoro-6-(2-(isoxazol-3-yl)vinyl)-1-(phenylsulfonyl)-1H-indol-2-yl)methyl)carbamate (1.30 g, 2.61 mmol, 1.00 eq) in MeOH (10 mL) and H₂O (2 mL) was added K₂CO₃ (1.08 g, 7.84 mmol, 3.00 eq) in one portion. The reaction mixture was heated and stirred at 90° C. for 12 h. After cooling to room temperature, the reaction mixture was quenched by addition of H₂O (10 mL), extracted with EtOAc (3×15 mL). The combined organic layers were washed with brine (10 mL), dried over Na₂SO₄, filtered, and concentrated under reduced pressure to give a residue. The residue was purified by column chromatography to give tert-butyl (E)-((5-fluoro-6-(2-(isoxazol-3-yl)vinyl)-1H-indol-2-yl)methyl)carbamate. LC-MS (ESI) m/z calcd for C₁₉H₂₀FN₃O₃: 357.15; found: 358.1 [M+H]⁺.

### Step 3: (E)-(5-fluoro-6-(2-(isoxazol-3-yl)vinyl)-1H-indol-2-yl)methanamine hydrochloride

To a solution of tert-butyl (E)-((5-fluoro-6-(2-(isoxazol-3-yl)vinyl)-1H-indol-2-yl)methyl)carbamate (850 mg, 2.38 mmol, 1.00 eq) in EtOAc (8 mL) was added HCl/EtOAc (4 M in EtOAc, 10 mL, 16.8 eq) in portions. The reaction mixture was stirred at 25° C. for 1 h. The reaction mixture was concentrated under reduced pressure directly to give (E)-(5-fluoro-6-(2-(isoxazol-3-yl)vinyl)-1H-indol-2-yl)methanamine hydrochloride which was used for next step directly without of further purification. LC-MS (ESI) m/z calcd for C₁₄H₁₂FN₃O: 257.10; found: 241.1 [M−NH₂+H]⁺.

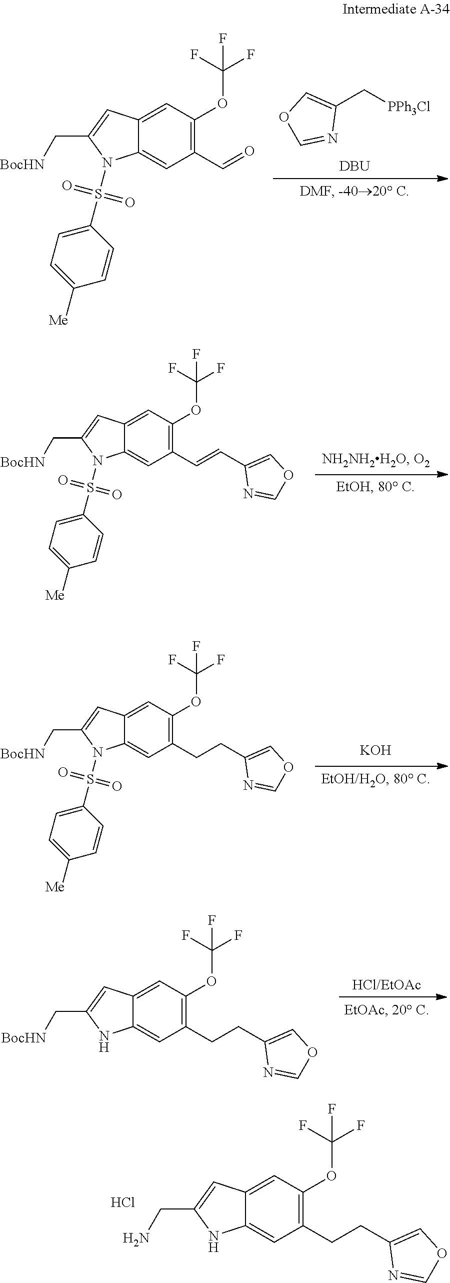

### Step 1: tert-butyl (E)-((6-(2-(oxazol-4-yl)vinyl)-1-tosyl-5-(trifluoromethoxy)-1H-indol-2-yl)methyl)carbamate

A mixture of tert-butyl ((6-formyl-1-tosyl-5-(trifluoromethoxy)-1H-indol-2-yl)methyl)carbamate (500 mg, 975 μmol, 1.00 eq), 4-((chlorotriphenyl-15-phosphanyl)methyl)oxazole (555 mg, 1.46 mmol, 1.50 eq) in DMF (0.5 mL) was degassed and purged with N₂ three times, then the mixture was cooled to −40° C. DBU (222 mg, 1.46 mmol, 1.50 eq) was added dropwise at −40° C., after the addition, the reaction mixture was stirred at 20° C. for 4 h under N₂ atmosphere. The reaction mixture was quenched by addition of H₂O (10 mL), extracted with EtOAc (3×10 mL). The combined organic layers were washed with brine (5 mL), dried over Na₂SO₄, filtered, and concentrated under reduced pressure to give a residue. The residue was purified by column chromatography to give tert-butyl (E)-((6-(2-(oxazol-4-yl)vinyl)-1-tosyl-5-(trifluoromethoxy)-1H-indol-2-yl)methyl)carbamate. LC-MS (ESI) m/z calcd for C₂₇H₂₆F₃N₃O₆S: 577.15; found 578.2 [M+H]⁺.

### Step 2: tert-butyl ((6-(2-(oxazol-4-yl)ethyl)-1-tosyl-5-(trifluoromethoxy)-1H-indol-2-yl)methyl)carbamate

To a solution of tert-butyl (E)-((6-(2-(oxazol-4-yl)vinyl)-1-tosyl-5-(trifluoromethoxy)-1H-indol-2-yl)methyl)carbamate (545 mg, 943 μmol, 1.00 eq) in EtOH (5 mL) was added NH₂NH₂—H₂O (1.31 g, 20.9 mmol, 80.0% purity, 20.0 eq) dropwise, then the mixture was degassed and purged with O₂ three times. The reaction mixture was heated and stirred at 80° C. for 3 h under O₂ (15 Psi) atmosphere. After cooling to room temperature, the reaction mixture was quenched by addition of H₂O (5 mL), extracted with EtOAc (3×10 mL). The combined organic layers were washed with brine (10 mL), dried over Na₂SO₄, filtered and concentrated under reduced pressure to give a residue. The residue was purified by to give tert-butyl ((6-(2-(oxazol-4-yl)ethyl)-1-tosyl-5-(trifluoromethoxy)-1H-indol-2-yl)methyl)carbamate. LC-MS (ESI) m/z calcd for C₂₇H₂₈F₃N₃O₆S: 579.17; found 602.3 [M+Na]⁺.

### Step 3: tert-butyl ((6-(2-(oxazol-4-yl)ethyl)-5-(trifluoromethoxy)-1H-indol-2-yl)methyl)carbamate

To a solution of tert-butyl ((6-(2-(oxazol-4-yl)ethyl)-1-tosyl-5-(trifluoromethoxy)-1H-indol-2-yl)methyl)carbamate (320 mg, 552 μmol, 1.00 eq) in EtOH (3 mL) and H₂O (0.6 mL) was added KOH (155 mg, 2.76 mmol, 5.00 eq) in one portion. The reaction mixture was heated and stirred at 80° C. for 1 h. After cooling to room temperature, the reaction mixture was quenched by addition H₂O (10 mL) and extracted with EtOAc (3×15 mL). The combined organic layers were washed with brine (10 mL), dried over Na₂SO₄, filtered, and concentrated under reduced pressure to give a residue. The residue was purified by column chromatography to give tert-butyl ((6-(2-(oxazol-4-yl)ethyl)-5-(trifluoromethoxy)-1H-indol-2-yl)methyl)carbamate. LC-MS (ESI) m/z calcd for C₂₀H₂₂F₃N₃O₄: 425.16; found 426.1 [M+H]⁺.

### Step 4: (6-(2-(oxazol-4-yl)ethyl)-5-(trifluoromethoxy)-1H-indol-2-yl)methanamine hydrochloride

To a solution of tert-butyl ((6-(2-(oxazol-4-yl)ethyl)-5-(trifluoromethoxy)-1H-indol-2-yl)methyl)carbamate (195 mg, 458 μmol, 1.00 eq) in EtOAc (1.5 mL) was added HCl/EtOAc (4 M, 3 mL, 26.1 eq) in portions at 0° C. The reaction mixture was stirred at 20° C. for 1 h. The reaction mixture was concentrated under reduced pressure directly to give (6-(2-(oxazol-4-yl)ethyl)-5-(trifluoromethoxy)-1H-indol-2-yl)methanamine hydrochloride, which was used for next step directly without of further purification. LC-MS (ESI) m/z calcd for C₁₅H₁₄F₃N₃O₂: 325.10; found 326.0 [M+H]⁺.

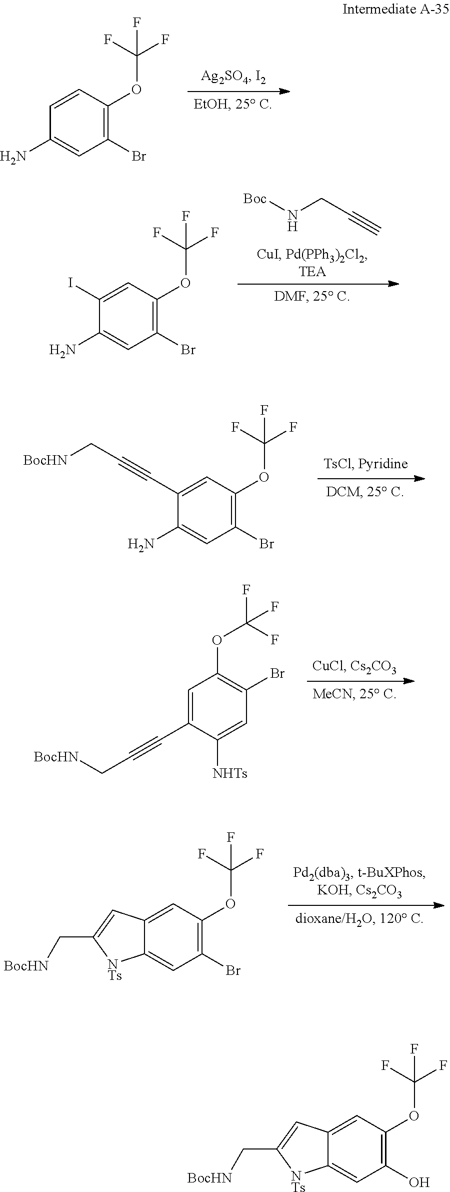

### Step 1: 5-bromo-2-iodo-4-(trifluoromethoxy)aniline

To a solution of 3-bromo-4-(trifluoromethoxy)aniline (25.0 g, 97.6 mmol, 1.00 eq) in EtOH (250 mL) were added 12 (136 mmol, 27.5 mL, 1.40 eq) and Ag₂SO₄ (45.6 g, 146 mmol, 1.50 eq) in portions at 25° C., the reaction mixture was stirred at 25° C. for 1 h. The reaction mixture was poured into sat. aq. Na₂SO₃ solution (200 mL) and stirred for 2 mins. The aqueous phase was extracted with ethyl acetate (3×200 mL). The combined organic phase was washed with brine (2×150 mL), dried with anhydrous Na₂SO₄, filtered, and concentrated under reduced pressure to give a residue. The residue was purified by column to give 5-bromo-2-iodo-4-(trifluoromethoxy)aniline. LC-MS (ESI) m/z calcd for C₇H₄BrF₃INO: 380.85; found: 381.8 [M+H]⁺.

### Step 2: tert-butyl (3-(2-amino-4-bromo-5-(trifluoromethoxy)phenyl)prop-2-yn-1-yl)carbamate

To a mixture of 5-bromo-2-iodo-4-(trifluoromethoxy)aniline (27.0 g, 70.7 mmol, 1.00 eq) and tert-butyl N-prop-2-ynylcarbamate (10.9 g, 70.7 mmol, 1.00 eq) in DMF (300 mL) was added TEA (353 mmol, 49.2 mL, 5.00 eq) in one portion, the system was degassed and purged with N₂ for 3 times. Then CuI (1.35 g, 7.07 mmol, 0.10 eq) and Pd(PPh₃)₂Cl₂ (1.49 g, 2.12 mmol, 0.03 eq) were added in one portion, and the reaction mixture was stirred at 25° C. for 1 h. The reaction mixture was quenched by addition of H₂O (300 mL) and extracted with EtOAc (3×100 mL). The combined organic layers were washed with brine (150 mL), dried over Na₂SO₄, filtered, and concentrated under reduced pressure to give a residue. The residue was purified by column chromatography to give tert-butyl (3-(2-amino-4-bromo-5-(trifluoromethoxy)phenyl)prop-2-yn-1-yl)carbamate. LC-MS (ESI) m/z calcd for C₁₅H₁₆BrF₃N₂O₃: 408.03; found: 409.1 [M+H]⁺.

### Step 3: tert-butyl (3-(4-bromo-2-((4-methylphenyl)sulfonamido)-5-(trifluoromethoxy)phenyl)prop-2-yn-1-yl)carbamate

To a solution of tert-butyl (3-(2-amino-4-bromo-5-(trifluoromethoxy)phenyl)prop-2-yn-1-yl)carbamate (11.0 g, 26.8 mmol, 1.00 eq) in DCM (110 mL) was added pyridine (53.7 mmol, 4.34 mL, 2.00 eq) and 4-methylbenzenesulfonyl chloride (TsCl, 5.64 g, 29.5 mmol, 1.10 eq) in one portion. The reaction mixture was stirred at 25° C. for 12 h. The reaction mixture was quenched by addition H₂O (150 mL), extracted with EtOAc (3×50 mL). The combined organic layers were washed with brine (100 mL), dried over Na₂SO₄, filtered, and concentrated under reduced pressure to give a residue. The residue was purified by column chromatography to give tert-butyl (3-(4-bromo-2-((4-methylphenyl)sulfonamido)-5-(trifluoromethoxy)phenyl)prop-2-yn-1-yl)carbamate. LC-MS (ESI) m/z calcd for C₂₂H₂₂BrF₃N₂O₅S: 562.04; found: 585.1 [M+Na]⁺.

### Step 4: tert-butyl ((6-bromo-1-tosyl-5-(trifluoromethoxy)-1H-indol-2-yl)methyl)carbamate

To a solution of tert-butyl (3-(4-bromo-2-((4-methylphenyl)sulfonamido)-5-(trifluoromethoxy)phenyl)prop-2-yn-1-yl)carbamate (10.0 g, 17.7 mmol, 1.00 eq) in MeCN (125 mL) were added CuCl (357 mg, 2.66 mmol, 0.15 eq) and Cs₂CO₃ (867 mg, 2.66 mmol, 0.15 eq) in one portion. The system was degassed and then charged with N₂ for three times. The reaction mixture was stirred at 25° C. for 4 h. Four parallel reactions were combined together for work up. The reaction mixture was quenched by addition of H₂O (150 mL), extracted with EtOAc (3×50 mL). The combined organic layers were washed with brine (100 mL) dried over Na₂SO₄, filtered, and concentrated under reduced pressure to give a residue. The residue was triturated with MTBE (20 mL) at 25° C. for 30 mins and filtered, and then the filter cake was collected and concentrated under reduced pressure to give tert-butyl ((6-bromo-1-tosyl-5-(trifluoromethoxy)-1H-indol-2-yl)methyl)carbamate.

### Step 5: tert-butyl ((6-hydroxy-1-tosyl-5-(trifluoromethoxy)-1H-indol-2-yl)methyl)carbamate

To a solution of tert-butyl ((6-bromo-1-tosyl-5-(trifluoromethoxy)-1H-indol-2-yl)methyl)carbamate (2.00 g, 3.55 mmol, 1.00 eq) in H₂O (4 mL) and dioxane (20 mL) were added KOH (796 mg, 14.2 mmol, 4.00 eq) and Cs₂CO₃ (2.31 g, 7.10 mmol, 2.00 eq) in one portion. The system was degassed and then charged with N₂ three times. Then t-BuXPhos (150 mg, 355 μmol, 0.10 eq) and Pd₂(dba)₃ (325 mg, 355 μmol, 0.10 eq) were added. The reaction mixture was heated and stirred at 120° C. for 2.5 h. After cooling to room temperature, the reaction mixture was quenched by addition of H₂O (30 mL) and extracted with EtOAc (3×10 mL). The combined organic layers were washed with brine (15 mL), dried over Na₂SO₄, filtered, and concentrated under reduced pressure to give a residue. The residue was purified by column chromatography to give tert-butyl ((6-hydroxy-1-tosyl-5-(trifluoromethoxy)-1H-indol-2-yl)methyl)carbamate. LC-MS (ESI) m/z calcd for C₂₂H₂₃F₃N₂O₆S: 500.12; found: 445.0 [M−tBu+H]⁺.

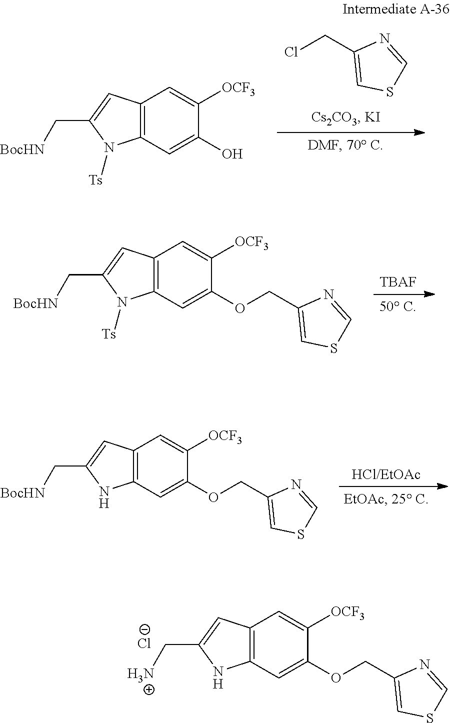

### Step 1: tert-butyl ((6-(thiazol-4-ylmethoxy)-1-tosyl-5-(trifluoromethoxy)-1H-indol-2-yl)methyl)carbamate

To a solution of tert-butyl ((6-hydroxy-1-tosyl-5-(trifluoromethoxy)-1H-indol-2-yl)methyl)carbamate (500 mg, 999 μmol, 1.00 eq) in DMF (5 mL) was added Cs₂CO₃ (651 mg, 2.00 mmol, 2.00 eq), KI (16.5 mg, 99.9 μmol, 0.10 eq) and 4-(chloromethyl)thiazole (160 mg, 1.20 mmol, 1.20 eq). The reaction mixture was heated to 70° C. for 1 h. After cooling to room temperature, the reaction mixture was quenched by addition H₂O (5 mL), extracted with EtOAc (3×5 mL). The combined organic layers were washed with brine (5 mL), dried over Na₂SO₄, filtered, and concentrated under reduced pressure. Purification by column chromatography gave tert-butyl ((6-(thiazol-4-ylmethoxy)-1-tosyl-5-(trifluoromethoxy)-1H-indol-2-yl)methyl)carbamate. LC-MS (ESI) m/z calcd for C₂₆H₂₆F₃N₃O₆S₂: 597.12; found: 542.1 [M−tBu+H]⁺.

### Step 2: tert-butyl ((6-(thiazol-4-ylmethoxy)-5-(trifluoromethoxy)-1H-indol-2-yl)methyl)carbamate

To tert-butyl ((6-(thiazol-4-ylmethoxy)-1-tosyl-5-(trifluoromethoxy)-1H-indol-2-yl)methyl)carbamate (500 mg, 836 μmol, 1.00 eq) was added TBAF (1 M in THF, 5 mL, 5.98 eq) dropwise at 20° C. The resulting reaction mixture was heated to 50° C. and stirred for 1 h. After cooling to room temperature, the reaction mixture was quenched by addition sat. aq. NH₄Cl (10 mL), extracted with EtOAc (3×5 mL). The combined organic phase was washed with brine (5 mL), dried over Na₂SO₄, filtered, and concentrated under reduced pressure to give tert-butyl ((6-(thiazol-4-ylmethoxy)-5-(trifluoromethoxy)-1H-indol-2-yl)methyl)carbamate, which was used directly in next step without of further purification. LC-MS (ESI) m/z calcd C₁₉H₂₀F₃N₃O₄S: 443.11; found: 444.1 [M+H]⁺.

### Step 3: (6-(thiazol-4-ylmethoxy)-5-(trifluoromethoxy)-1H-indol-2-yl)methanamine

To a solution of tert-butyl ((6-(thiazol-4-ylmethoxy)-5-(trifluoromethoxy)-1H-indol-2-yl)methyl)carbamate (400 mg, 902 μmol, 1.00 eq) in EtOAc (2 mL) was added HCl/EtOAc (4 M, 10 mL, 44.3 eq) in portions. The resulting reaction mixture was stirred at 25° C. for 1 h. The reaction mixture was concentrated in vacuo. The resulting residue was triturated with EtOAc (10 mL) at 25° C. for 30 mins then filtered, the filter cake was collected to give (6-(thiazol-4-ylmethoxy)-5-(trifluoromethoxy)-1H-indol-2-yl)methanamine hydrochloride. LC-MS (ESI) m/z calcd for C₁₄H₁₂F₃N₃O₂S: 343.06; found: 344.0 [M+H]⁺.

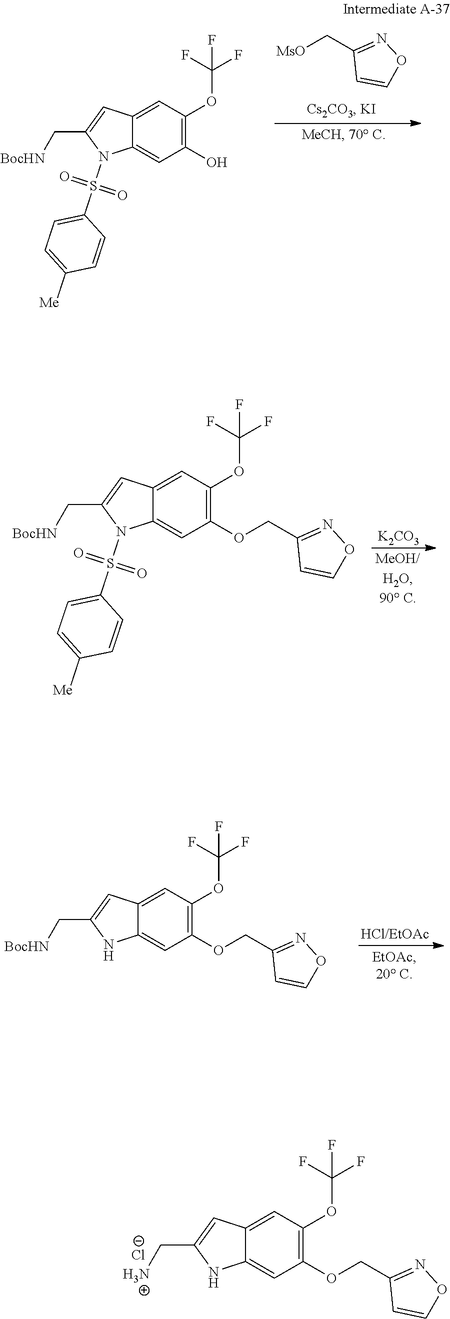

### Step 1: tert-butyl ((6-(isoxazol-3-ylmethoxy)-1-tosyl-5-(trifluoromethoxy)-1H-indol-2-yl)methyl)carbamate

To a solution of isoxazol-3-ylmethyl methanesulfonate (212 mg, 1.20 mmol, 1.20 eq) and tert-butyl ((6-hydroxy-1-tosyl-5-(trifluoromethoxy)-1H-indol-2-yl)methyl)carbamate (500 mg, 999 mol, 1.00 eq) in MeCN (10 mL) was added Cs₂CO₃ (651 mg, 2.00 mmol, 2.00 eq) and KI (166 mg, 999 μmol, 1.00 eq) in one portion. The reaction mixture was heated and stirred at 70° C. for 2 h. After cooling to room temperature, the reaction mixture was quenched by addition of H₂O (20 mL), extracted with EtOAc (3×25 mL). The combined organic layers were dried over Na₂SO₄, filtered, and concentrated under reduced pressure to dryness to give a residue. The residue was purified by column chromatography to give tert-butyl ((6-(isoxazol-3-ylmethoxy)-1-tosyl-5-(trifluoromethoxy)-1H-indol-2-yl)methyl)carbamate. LC-MS (ESI) m/z calcd for C₂₆H₂₆F₃N₃O₇S: 581.14; found: 604.1 [M+Na]⁺.

### Step 2: tert-butyl ((6-(isoxazol-3-ylmethoxy)-5-(trifluoromethoxy)-1H-indol-2-yl)methyl)carbamate

To a solution of tert-butyl ((6-(isoxazol-3-ylmethoxy)-1-tosyl-5-(trifluoromethoxy)-1H-indol-2-yl)methyl)carbamate (520 mg, 894 μmol, 1.00 eq) in MeOH (9 mL) and H₂O (3 mL) was added K₂CO₃ (247.15 mg, 1.79 mmol, 2.00 eq) in one portion at 20° C. Then the reaction mixture was heated and stirred at 90° C. for 16 h. After cooling to room temperature, the reaction was quenched by addition of H₂O (20 mL), then extracted with EtOAc (3×30 mL). The combined organic layers were dried over Na₂SO₄, filtered, and concentrated under reduced pressure to dryness to give a residue. The residue was purified by column chromatography to give tert-butyl ((6-(isoxazol-3-ylmethoxy)-5-(trifluoromethoxy)-1H-indol-2-yl)methyl)carbamate. LC-MS (ESI) m/z calcd for C₁₉H₂₀F₃N₃O₅: 427.14; found: 428.1 [M+H]⁺.

### Step 3: (6-(isoxazol-3-ylmethoxy)-5-(trifluoromethoxy)-1H-indol-2-yl)methanamine hydrochloride

To a solution of tert-butyl ((6-(isoxazol-3-ylmethoxy)-5-(trifluoromethoxy)-1H-indol-2-yl)methyl)carbamate (290 mg, 679 μmol, 1.00 eq) in EtOAc (5 mL) was added HCl/EtOAc (4 M, 4.83 mL, 28.5 eq) in portions at 0° C. The reaction mixture was stirred at 20° C. for 30 min. The reaction mixture was concentrated under reduced pressure to give (6-(isoxazol-3-ylmethoxy)-5-(trifluoromethoxy)-1H-indol-2-yl)methanamine hydrochloride, which was used for next step directly without of further purification. LC-MS (ESI) m/z calcd for C₁₄H₁₃ClF₃N₃O₃: 327.08; found: 310.9 [M−NH₂+H]⁺.

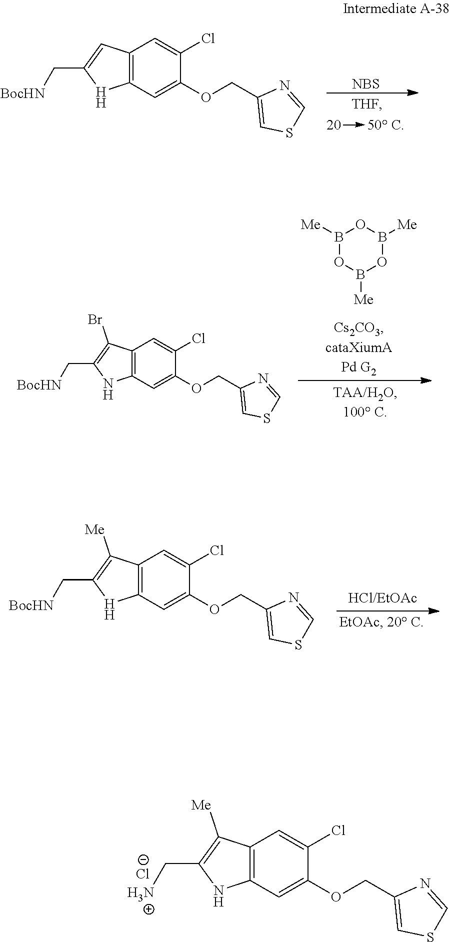

### Step 1: tert-butyl ((5-chloro-6-(thiazol-4-ylmethoxy)-1H-indol-2-yl)methyl)carbamate

To a solution of tert-butyl ((5-chloro-6-(thiazol-4-ylmethoxy)-1H-indol-2-yl) methyl) carbamate (500 mg, 1.27 mmol, 1.00 eq) in THF (5 mL) was added NBS (237 mg, 1.33 mmol, 1.05 eq) in one portion at 20° C., then the reaction mixture was heated to 50° C. and stirred at 50° C. for 2 h. After cooling to room temperature, the reaction mixture was quenched by addition of sat. aq. Na₂S₂O₃ solution (15 mL) at 20° C., then the aqueous phase was extracted with ethyl acetate (3×30 ml). The combined organic phases were washed with brine (2×20 mL), dried over Na₂SO₄, filtered, and concentrated under reduced pressure to give tert-butyl ((3-bromo-5-chloro-6-(thiazol-4-ylmethoxy)-1H-indol-2-yl) methyl) carbamate, which was used for next step directly without of further purification. LC-MS (ESI) m/z calcd for C₁₈H₁₉BrClN₃O₃S: 471.00; found 472.0 [M+H]⁺.

### Step 2: tert-butyl ((5-chloro-3-methyl-6-(thiazol-4-ylmethoxy)-1H-indol-2-yl) methyl) carbamate

To a mixture of 2,4,6-trimethyl-1,3,5,2,4,6-trioxatriborinane (4.86 mmol, 1.36 mL, 5.00 eq) and tert-butyl ((3-bromo-5-chloro-6-(thiazol-4-ylmethoxy)-1H-indol-2-yl) methyl) carbamate (460 mg, 972 μmol, 1.00 eq) in H₂O (1 mL) and 2-methylbutan-2-ol (5 mL) was added Cs₂CO₃ (634 mg, 1.95 mmol, 2.00 eq) in one portion, and the reaction system was degassed and then charged with nitrogen three times. CataCXiumA Pd G2 (65.0 mg, 97.3 μmol, 0.10 eq) was then added in one portion. The reaction mixture was heated to 100° C. and stirred at 100° C. for 12 h. After cooling to room temperature, the reaction mixture was quenched by addition of H₂O (15 mL) and extracted with ethyl acetate (3×30 ml). The combined organic phases were washed with brine (2×20 mL), dried over Na₂SO₄, filtered, and concentrated under reduced pressure to give a residue. The residue was purified by column chromatography to give tert-butyl ((5-chloro-3-methyl-6-(thiazol-4-ylmethoxy)-1H-indol-2-yl) methyl) carbamate. LC-MS (ESI) m/z calcd for C₁₉H₂₂ClN₃O₃S: 407.11; found 408.1 [M+H]⁺.

### Step 3: (5-chloro-3-methyl-6-(thiazol-4-ylmethoxy)-1H-indol-2-yl) methanamine hydrochloride

To a solution of tert-butyl ((5-chloro-3-methyl-6-(thiazol-4-ylmethoxy)-1H-indol-2-yl) methyl) carbamate (450 mg, 1.10 mmol, 1.00 eq) in ethyl acetate (0.2 mL) was added HCl (4 M in EtOAc, 2.76 mL, 10.0 eq) in one portion, then the reaction mixture was stirred at 20° C. for 1 h. The reaction mixture was concentrated under reduced pressure directly to give (5-chloro-3-methyl-6-(thiazol-4-ylmethoxy)-1H-indol-2-yl) methanamine hydrochloride, which was used for next step directly without further purification. LC-MS (ESI) m/z calcd for C₁₉H₂₂ClN₃O₃S: 307.05; found 291.1 [M−NH₂+H]⁺.

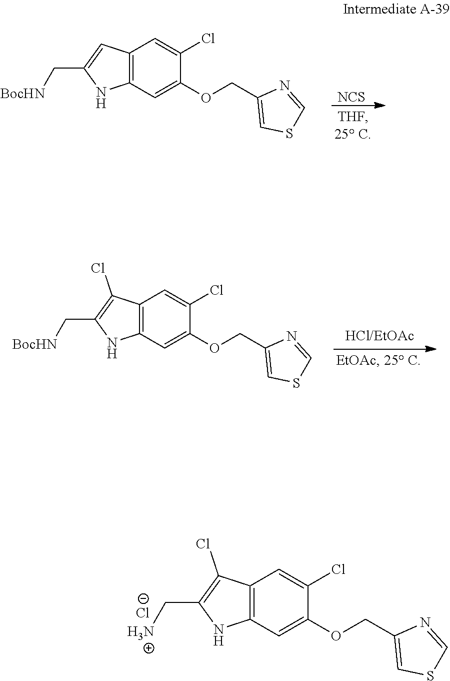

### Step 1: tert-butyl ((3,5-dichloro-6-(thiazol-4-ylmethoxy)-1H-indol-2-yl)methyl)carbamate

To a solution of tert-butyl ((5-chloro-6-(thiazol-4-ylmethoxy)-1H-indol-2-yl) methyl) carbamate (200 mg, 507 μmol, 1.00 eq) in THF (3 mL) was added 1-chloropyrrolidine-2,5-dione (NCS, 101 mg, 761 μmol, 1.50 eq) in portions at 25° C. The reaction mixture was stirred at 25° C. for 2 h. The reaction mixture was quenched by addition of H₂O (10 mL), extracted with ethyl acetate (3×15 mL). The combined organic phase was washed with brine (10 mL), dried over Na₂SO₄, filtered, and concentrated under reduced pressure. Purification by column chromatography gave tert-butyl ((3,5-dichloro-6-(thiazol-4-ylmethoxy)-1H-indol-2-yl)methyl) carbamate (200 mg). LC-MS (ESI) m/z calcd for C₁₈H₁₉Cl₂N₃O₃S: 427.05; found: 428.2 [M+H]⁺.

### Step 2: (3,5-dichloro-6-(thiazol-4-ylmethoxy)-1H-indol-2-yl)methanamine

To a solution of tert-butyl ((3,5-dichloro-6-(thiazol-4-ylmethoxy)-1H-indol-2-yl) methyl) carbamate (200 mg, 466 μmol, 1.00 eq) in ethyl acetate (2 mL) was added HCl/ethyl acetate (4M, 350 μL, 3.00 eq) dropwise. The reaction mixture was stirred at 25° C. for 1 h. The reaction mixture was concentrated under reduced pressure to give (3,5-dichloro-6-(thiazol-4-ylmethoxy)-1H-indol-2-yl) methanamine hydrochloride which was used directly in next step without further purification. LC-MS (ESI) m/z calcd for C₁₃H₁₁Cl₂N₃OS: 327.00; found: 311.2 [M−NH₂+H]⁺.

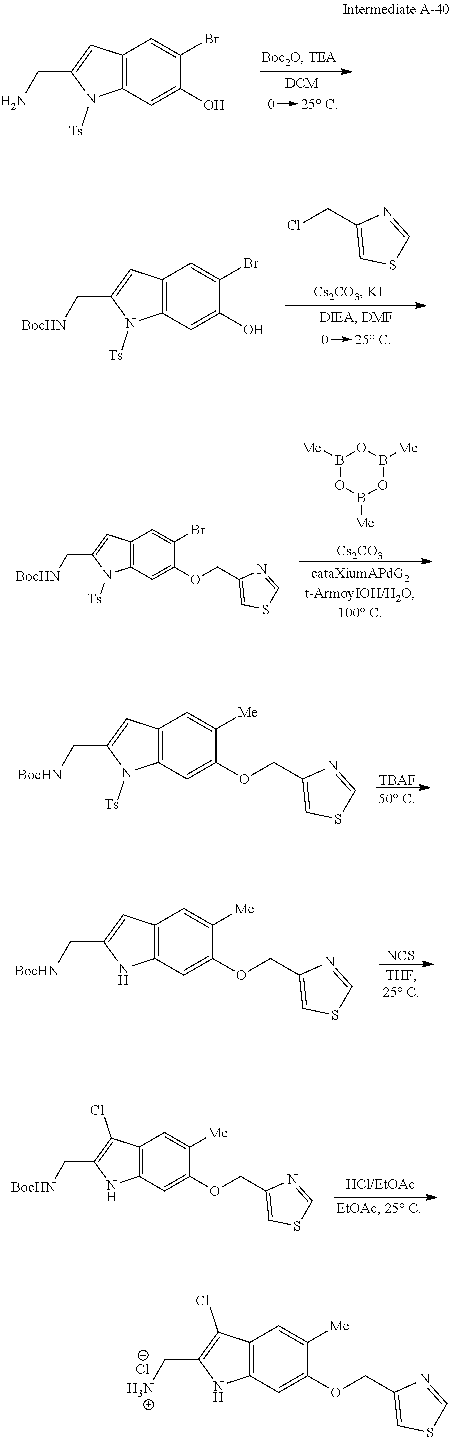

### Step 1: tert-butyl ((5-bromo-6-hydroxy-1-tosyl-1H-indol-2-yl)methyl)carbamate

To a solution of 2-(aminomethyl)-5-bromo-1-tosyl-1H-indol-6-ol (3.70 g, 7.77 mmol, 1.00 eq, HBr salt) in DCM (37 mL) was added TEA (1.18 g, 11.6 mmol, 1.50 eq) and tert-butoxycarbonyl tert-butyl carbonate (1.36 g, 6.22 mmol, 0.80 eq) in portions at 0° C. Then the reaction mixture was allowed to warm to 25° C. and stirred at 25° C. for 2 h. The reaction mixture was poured into water (15 mL) and stirred for 2 min. The aqueous phase was extracted with ethyl acetate (3×25 mL). The combined organic phase was washed with brine (2×15 mL), dried over anhydrous Na₂SO₄, filtered, and concentrated under reduced pressure to give a residue. Purification by column chromatography gave tert-butyl ((5-bromo-6-hydroxy-1-tosyl-1H-indol-2-yl)methyl)carbamate. LC-MS (ESI) m/z calcd for C₂₁H₂₃BrN₂O₅S: 494.05; found: 517.0 [M+Na]⁺.

### Step 2: tert-butyl ((5-bromo-6-(thiazol-4-ylmethoxy)-1-tosyl-1H-indol-2-yl)methyl)carbamate

To a solution of tert-butyl ((5-bromo-6-hydroxy-1-tosyl-1H-indol-2-yl)methyl)carbamate (2.70 g, 5.45 mmol, 1.00 eq) in DMF (27 mL) was added a solution of 4-(chloromethyl)thiazole (1.20 g, 7.09 mmol, 1.30 eq, HCl salt) and DIEA (704 mg, 5.45 mmol, 949 μL, 1.00 eq) in DMF (5 mL) dropwise at 25° C. Then KI (90.4 mg, 545 μmol, 0.10 eq) and Cs₂CO₃ (3.55 g, 10.9 mmol, 2.00 eq) were added. The reaction mixture was heated to 70° C. and stirred at 70° C. for 2 h. After cooling to room temperature, the reaction mixture was poured into H₂O (30 mL). The aqueous phase was extracted with ethyl acetate (3×20 mL). The combined organic phase was washed with brine (2×15 mL), dried over anhydrous Na₂SO₄, filtered, and concentrated under reduced pressure to give a residue. Purification by column chromatography gave tert-butyl ((5-bromo-6-(thiazol-4-ylmethoxy)-1-tosyl-1H-indol-2-yl)methyl)carbamate. LC-MS (ESI) m/z calcd for C₂₅H₂₆BrN₃O₅S₂: 591.05; found: 536.0 [M−tBu+H⁺].

### Step 3: tert-butyl ((5-methyl-6-(thiazol-4-ylmethoxy)-1-tosyl-1H-indol-2-yl)methyl)carbamate

To a mixture of tert-butyl ((5-bromo-6-(thiazol-4-ylmethoxy)-1-tosyl-1H-indol-2-yl)methyl)carbamate (1.00 g, 1.69 mmol, 1.00 eq) and 2,4,6-trimethyl-1,3,5,2,4,6-trioxatriborinane (2.12 g, 8.44 mmol, 5.00 eq) in H₂O (4 mL) and 2-methylbutan-2-ol (20 mL) was added Cs₂CO₃ (1.10 g, 3.38 mmol, 2.00 eq). The system was degassed and purged with N₂ three times. CataXiumA Pd G2 (113 mg, 168 μmol, 0.100 eq) was added, then the reaction mixture was heated to 100° C. and stirred for 12 h. After cooling to room temperature, the reaction mixture was poured into water (30 mL) and stirred for 2 min. The aqueous phase was extracted with ethyl acetate (3×30 mL). The combined organic phase was washed with brine (2×20 mL), dried over anhydrous Na₂SO₄, filtered, and concentrated under reduced pressure to give a residue. Purification by column chromatography gave tert-butyl ((5-methyl-6-(thiazol-4-ylmethoxy)-1-tosyl-1H-indol-2-yl)methyl)carbamate. LC-MS (ESI) m/z calcd C₂₆H₂₉N₃O₅S₂: 527.15; found: 472.1 [M−tBu+H]⁺.

### Step 4: tert-butyl ((5-methyl-6-(thiazol-4-ylmethoxy)-1H-indol-2-yl)methyl)carbamate

A solution of tert-butyl ((5-methyl-6-(thiazol-4-ylmethoxy)-1H-indol-2-yl)methyl)carbamate (1.10 g, 2.08 mmol, 1.00 eq) in TBAF (1.00 M in THF, 41.7 mL, 20.0 eq) was heated to 50° C. and for 12 h. After cooling to room temperature, the reaction mixture was poured into water (30 mL). The aqueous phase was extracted with ethyl acetate (3×20 mL). The combined organic phase was washed with sat. aq. NH₄Cl solution (3×15 mL), dried over anhydrous Na₂SO₄, filtered, and concentrated under reduced pressure to give a residue. Purification by column chromatography gave tert-butyl ((5-methyl-6-(thiazol-4-ylmethoxy)-1H-indol-2-yl) methyl) carbamate. LC-MS (ESI) m/z calcd for C₁₉H₂₃N₃O₃S: 373.15; found: 374.1 [M+H]⁺.

### Step 5: tert-butyl ((3-chloro-5-methyl-6-(thiazol-4-ylmethoxy)-1H-indol-2-yl)methyl)carbamate

To a solution of tert-butyl ((5-methyl-6-(thiazol-4-ylmethoxy)-1H-indol-2-yl)methyl)carbamate (300 mg, 803 μmol, 1.00 eq) in THF (3 mL) was added 1-chloropyrrolidine-2,5-dione (NCS, 161 mg, 1.20 mmol, 1.50 eq) in portions at 25° C. The reaction mixture was stirred at 25° C. for 0.5 h. The reaction mixture was quenched by addition of H₂O (2 mL), extracted with ethyl acetate (3×5 mL). The combined organic phase was dried over anhydrous Na₂SO₄, filtered, and concentrated under reduced pressure to give a residue. Purification by column chromatography gave tert-butyl ((3-chloro-5-methyl-6-(thiazol-4-ylmethoxy)-1H-indol-2-yl)methyl)carbamate. LC-MS (ESI) m/z calcd for C₁₉H₂₂ClN₃O₃S: 407.11; found: 408.3 [M+H]⁺.

### Step 6: (3-chloro-5-methyl-6-(thiazol-4-ylmethoxy)-1H-indol-2-yl)methanamine hydrochloride

To a solution of tert-butyl ((3-chloro-5-methyl-6-(thiazol-4-ylmethoxy)-1H-indol-2-yl)methyl)carbamate (180 mg, 441 μmol, 1.00 eq) in EtOAc (0.50 mL) was added HCl (4.00 M in EtOAc, 2.00 mL, 18.1 eq) in portions, then the reaction mixture was stirred at 25° C. for 0.5 h. The reaction mixture was concentrated under reduced pressure to give (3-chloro-5-methyl-6-(thiazol-4-ylmethoxy)-1H-indol-2-yl)methanamine hydrochloride which was used for next step directly without further purification. LC-MS (ESI) m/z calcd for C₁₄H₁₄ClN₃OS: 307.05; found: 291.2 [M−NH₂]⁺.

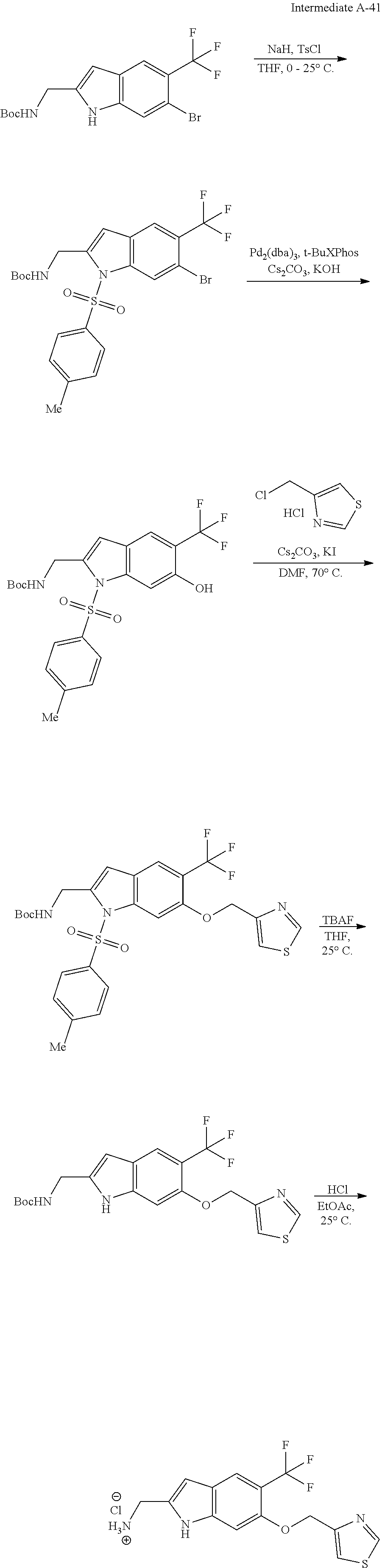

### Step 1: tert-butyl ((6-bromo-1-tosyl-5-(trifluoromethyl)-1H-indol-2-yl)methyl)carbamate

Two reactions were carried out in parallel. To a solution of tert-butyl ((6-bromo-5-(trifluoromethyl)-1H-indol-2-yl)methyl)carbamate (1.50 g, 3.81 mmol, 1.00 eq) in THF (15 mL) at 0° C. was added NaH (305 mg, 7.63 mmol, 2.00 eq) in portions. After 30 min at 0° C. 4-methylbenzenesulfonyl chloride (872 mg, 4.58 mmol, 1.20 eq) was added in portions. The reaction mixture was allowed to warm to 25° C. After 30 min the two reactions were combined. The reaction mixture was quenched by addition of H₂O (20 mL) at 0° C. The solution was extracted with ethyl acetate (3×20 mL). The combined organic layers were washed with brine (60 mL), dried over anhydrous Na₂SO₄, filtered, and concentrated under reduced pressure. Trituration with petroleum ether (20 mL) at 25° C. for 30 min followed by filtration gave tert-butyl ((6-bromo-1-tosyl-5-(trifluoromethyl)-1H-indol-2-yl)methyl)carbamate. LC-MS (ESI) m/z calcd for C₂₂H₂₂BrF₃N₂O₄S: 546.0; found: 430.1 [M−NHBoc]⁺.

### Step 2: tert-butyl ((6-hydroxy-1-tosyl-5-(trifluoromethyl)-1H-indol-2-yl)methyl)carbamate

Two reactions were carried out in parallel. To a solution of tert-butyl ((6-bromo-1-tosyl-5-(trifluoromethyl)-1H-indol-2-yl)methyl)carbamate (1.60 g, 2.92 mmol, 1.00 eq) in H₂O (3.6 mL) and dioxane (18 mL) was added Cs₂CO₃ (1.90 g, 5.85 mmol, 2.00 eq), KOH (655 mg, 11.6 mmol, 4.00 eq), Pd₂(dba)₃ (267 mg, 292 μmol, 0.10 eq), and ditert-butyl-[2-(2,4,6-triisopropylphenyl) phenyl]phosphane (124 mg, 292 μmol, 0.10 eq). The reaction mixture was degassed and purged with N₂ three times then heated to 100° C. After 2 h the two reactions were cooled to 25° C. and combined for work up. The reaction mixture was quenched with H₂O (20 mL). The solution was extracted with ethyl acetate (3×20 mL). The combined organic layers were washed with brine (60 mL), dried over anhydrous Na₂SO₄, filtered, and concentrated under reduced pressure. Purification by column chromatography gave tert-butyl ((6-hydroxy-1-tosyl-5-(trifluoromethyl)-1H-indol-2-yl)methyl)carbamate. LC-MS (ESI) m/z calcd for C₂₂H₂₃F₃N₂O₅S: 484.1; found: 368.1 [M−NHBoc]⁺.

### Step 3: tert-butyl ((6-(thiazol-4-ylmethoxy)-1-tosyl-5-(trifluoromethyl)-1H-indol-2-yl)methyl)carbamate

To a solution of tert-butyl N-[[6-hydroxy-1-(p-tolylsulfonyl)-5-(trifluoromethyl)indol-2-yl]methyl]carbamate (270 mg, 557 μmol, 1.00 eq) in DMF (3.00 mL) were added 4-(chloromethyl)thiazole (113 mg, 668 μmol, 1.20 eq, HCl), KI (6.48 mg, 39.0 μmol, 0.07 eq), and Cs₂CO₃ (363 mg, 1.11 mmol, 2.00 eq). The reaction mixture was heated to 70° C. After 1 h the reaction mixture was quenched with H₂O (5 mL) at 25° C. The solution was extracted with ethyl acetate (3×5 mL). The combined organic layers were washed with brine (5 mL), dried over anhydrous Na₂SO₄, filtered, and concentrated under reduced pressure. Purification by prep-TLC gave tert-butyl N-[[1-(p-tolylsulfonyl)-6-(thiazol-4-ylmethoxy)-5-(trifluoromethyl)indol-2-yl]methyl]carbamate. LC-MS (ESI) m/z calcd for C₂₆H₂₆F₃N₃O₅S₂: 581.1; found: 582.3 [M+H]⁺.

### Step 4: tert-butyl ((6-(thiazol-4-ylmethoxy)-5-(trifluoromethyl)-1H-indol-2-yl)methyl)carbamate

To a solution of tert-butyl N-[[1-(p-tolylsulfonyl)-6-(thiazol-4-ylmethoxy)-5-(trifluoromethyl)indol-2-yl]methyl]carbamate (200 mg, 343 μmol, 1.00 eq) in THF (2.00 mL) was added TBAF (1 M in THF, 3.44 mL, 10.0 eq) in portions at 25° C. After 3 h the reaction mixture was poured into water (5 mL). After 2 min the solution was extracted with ethyl acetate (2×5 mL). The combined organic layers were washed with brine (2×5 mL), dried over anhydrous Na₂SO₄, filtered, and concentrated under reduced pressure. Purification by prep-TLC gave tert-butyl N-[[6-(thiazol-4-ylmethoxy)-5-(trifluoromethyl)-1H-indol-2-yl]methyl]carbamate. LC-MS (ESI) m/z calcd for C₁₉H₂₀F₃N₃O₃S: 427.1; found: 428.2 [M+H]⁺.

### Step 5: (6-(thiazol-4-ylmethoxy)-5-(trifluoromethyl)-1H-indol-2-yl)methanamine hydrochloride

To a solution of tert-butyl N-[[6-(thiazol-4-ylmethoxy)-5-(trifluoromethyl)-1H-indol-2-yl]methyl]carbamate (110 mg, 257 μmol, 1.00 eq) in EtOAc (2.00 mL) was added HCl in ethyl acetate (4 M, 10 mL, 155 eq) dropwise. The reaction mixture was stirred at 25° C. for 1 h. The reaction mixture was directly concentrated under reduced pressure to give [6-(thiazol-4-ylmethoxy)-5-(trifluoromethyl)-1H-indol-2-yl]methanamine hydrochloride. LC-MS (ESI) m/z calcd for C₁₄H₁₂F₃N₃OS: 327.1; found: 328.0 [M+H]⁺.

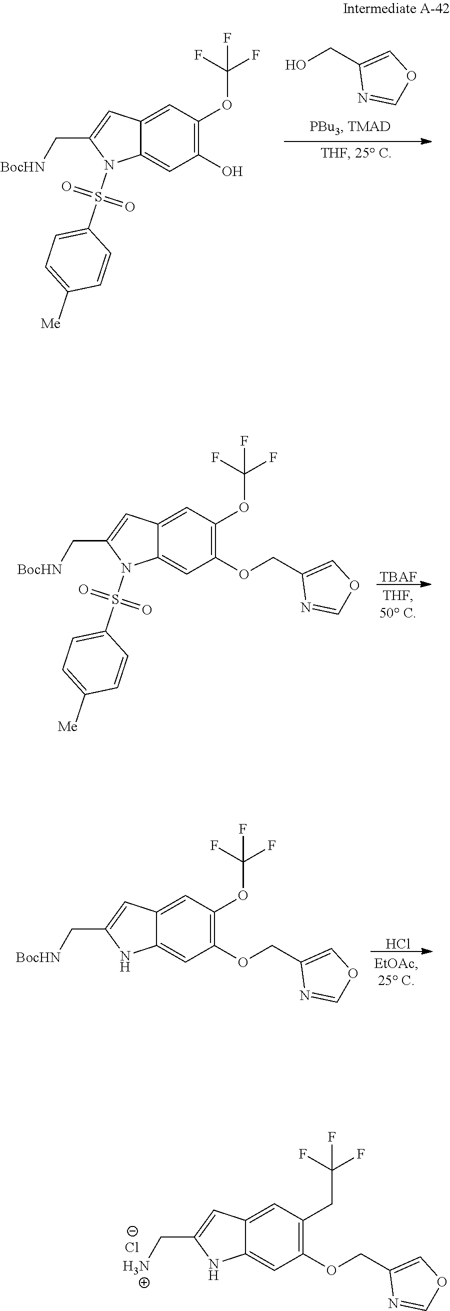

### Step 1: tert-butyl ((6-(oxazol-4-ylmethoxy)-1-tosyl-5-(trifluoromethoxy)-1H-indol-2-yl)methyl)carbamate

To a solution of tert-butyl N-[[6-hydroxy-1-(p-tolylsulfonyl)-5-(trifluoromethoxy)indol-2-yl]methyl]carbamate (700 mg, 1.40 mmol, 1.00 eq) in THF (10 mL) was added oxazol-4-ylmethanol (138 mg, 1.40 mmol, 1.00 eq), (3E)-3-(dimethylcarbamoylimino)-1,1-dimethylurea (TMAD, 722 mg, 4.20 mmol, 3.00 eq) and tributylphosphine (848 mg, 4.20 mmol, 1.04 mL, 3.00 eq) in portions. The reaction mixture was stirred at 25° C. for 16 h. The reaction mixture was quenched by addition of H₂O (30 mL) at 25° C. and extracted with ethyl acetate (3×30 mL). The combined organic phase was washed with brine (3×30 mL), dried over anhydrous Na₂SO₄, filtered, and concentrated under reduced pressure. Purification by column chromatography gave tert-butyl ((6-(oxazol-4-ylmethoxy)-1-tosyl-5-(trifluoromethoxy)-1H-indol-2-yl)methyl)carbamate. LC-MS (ESI) m/z calcd for C₂₆H₂₆F₃N₃O₇S: 581.14; found: 604.2 [M+Na]⁺.

### Step 2: tert-butyl ((6-(oxazol-4-ylmethoxy)-5-(trifluoromethoxy)-1H-indol-2-yl)methyl)carbamate

To a solution of tert-butyl ((6-(oxazol-4-ylmethoxy)-1-tosyl-5-(trifluoromethoxy)-1H-indol-2-yl)methyl)carbamate (300 mg, 515 μmol, 1.00 eq) in THF (1 mL) was added TBAF (1 M in THF, 2.58 mL, 5.00 eq) in portions at 25° C. under an atmosphere of N₂. The reaction mixture was heated and stirred at 50° C. for 2 h. The reaction mixture was allowed to cool to room temperature and quenched by addition of H₂O (10 mL), extracted with ethyl acetate (3×10 mL). The combined organic phase was washed with brine (3×10 mL), dried over anhydrous Na₂SO₄, filtered, and concentrated under reduced pressure. Purification by silica gel chromatography gave tert-butyl ((6-(oxazol-4-ylmethoxy)-5-(trifluoromethoxy)-1H-indol-2-yl)methyl)carbamate. LC-MS (ESI) m/z calcd for C₁₉H₂₀F₃N₃O₅: 427.14; found: 428.2 [M+H]⁺.

### Step 3: (6-(oxazol-4-ylmethoxy)-5-(trifluoromethoxy)-1H-indol-2-yl)methanamine hydrochloride

To a solution of tert-butyl ((6-(oxazol-4-ylmethoxy)-5-(trifluoromethoxy)-1H-indol-2-yl)methyl)carbamate (200 mg, 467 μmol, 1.00 eq) in EtOAc (1.5 mL) was added HCl (4 M in EtOAc, 10 mL, 85.4 eq) in portions. The reaction mixture was stirred at 25° C. for 1 h. Concentration under reduced pressure gave [6-(oxazol-4-ylmethoxy)-5-(trifluoromethoxy)-1H-indol-2-yl]methanamine hydrochloride, which was used without further purification. LC-MS (ESI) m/z calcd for C₁₄H₁₂F₃N₃O₃: 327.08; found: 311.0 [M−NH₂]⁺.

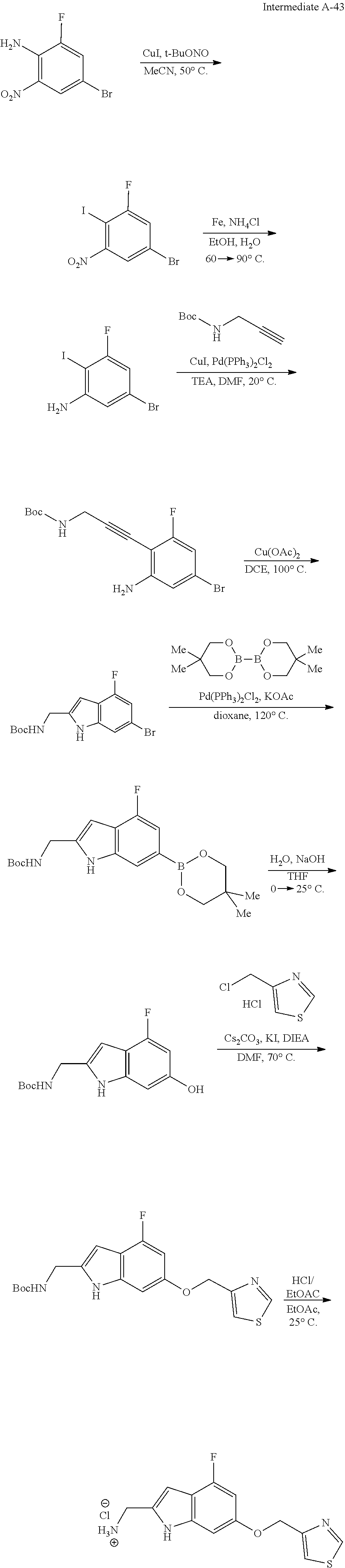

### Step 1: 5-bromo-1-fluoro-2-iodo-3-nitrobenzene

To a solution of 4-bromo-2-fluoro-6-nitroaniline (5.00 g, 21.2 mmol, 1.00 eq) in MeCN (5 mL) was added CuI (6.08 g, 31.9 mmol, 1.50 eq) and tert-butyl nitrite (3.29 g, 31.9 mmol, 1.50 eq) under N₂ atmosphere. The reaction mixture was degassed and purged with N₂ three times, then heated to 50° C. for 16 h under N₂ atmosphere. After cooling to room temperature, the reaction mixture was quenched with water (20 mL), extracted with ethyl acetate (2×30 mL). The combined organic phase was washed with brine (60 mL), dried over anhydrous Na₂SO₄, filtered, and concentrated under reduced pressure. Purification by column chromatography gave 5-bromo-1-fluoro-2-iodo-3-nitrobenzene.

### Step 2: 5-bromo-3-fluoro-2-iodoaniline

To a solution of 5-bromo-1-fluoro-2-iodo-3-nitrobenzene (4.00 g, 11.5 mmol, 1.00 eq) in EtOH (40 mL) and H₂O (10 mL) was added NH₄Cl (2.47 g, 46.2 mmol, 4.00 eq) at 20° C. before being heated to 60° C. Then, Fe (6.46 g, 115 mmol, 10.0 eq) was added in portions. The reaction mixture was heated to 90° C. for 1 h under N₂ atmosphere. After cooling to room temperature, the reaction mixture was filtered through Celite, and the filtrate was collected and diluted with water (10 mL). The aqueous phase was extracted with ethyl acetate (2×30 mL). The combined organic phase was washed with brine (60 mL), dried over anhydrous Na₂SO₄, filtered, and concentrated under reduced pressure. Purification by column chromatography gave 5-bromo-3-fluoro-2-iodoaniline. LC-MS (ESI) m/z calcd for C₆H₄BrFIN: 314.86; found: 315.9 [M+H]⁺.

### Step 3: tert-butyl (3-(2-amino-4-bromo-6-fluorophenyl)prop-2-yn-1-yl)carbamate

To a mixture of 5-bromo-3-fluoro-2-iodoaniline (6.50 g, 20.5 mmol, 1.00 eq) and tert-butyl N-prop-2-ynylcarbamate (4.79 g, 30.8 mmol, 1.50 eq) in DMF (65 mL) was added TEA (10.4 g, 102 mmol, 5.00 eq). The resulting mixture was degassed and purged with N₂ three times. Then, CuI (783 mg, 4.12 mmol, 0.20 eq) and Pd(PPh₃)₂Cl₂ (1.44 g, 2.06 mmol, 0.10 eq) were added 20° C. under N₂, the reaction mixture was degassed and purged with N₂ 3 times. The reaction mixture was stirred at 25° C. for 16 h. H₂O (10 mL) was added to quench before being extracted with ethyl acetate (3×20 mL). The combined organic phase was washed with brine (60 mL), dried over Na₂SO₄, filtered, and concentrated under reduced pressure. Purification by column chromatography gave tert-butyl (3-(2-amino-4-bromo-6-fluorophenyl)prop-2-yn-1-yl)carbamate (4.50 g). LC-MS (ESI) m/z calcd C₁₄H₁₆BrFN₂O₂: 342.04; found: 287.0 [M−tBu+H]⁺.

### Step 4: tert-butyl ((6-bromo-4-fluoro-1H-indol-2-yl)methyl)carbamate

To a solution of tert-butyl (3-(2-amino-4-bromo-6-fluorophenyl)prop-2-yn-1-yl)carbamate (2.30 g, 6.70 mmol, 1.00 eq) in 1,2-dichloroethane (23 mL) was added Cu(OAc)₂ (608 mg, 3.35 mmol, 0.50 eq). The reaction mixture was heated and stirred at 100° C. for 16 h. After cooling to room temperature, the reaction mixture was filtrated through celite and the filtrate concentrated under reduced pressure. The resulting residue was purified by column chromatography to give tert-butyl ((6-bromo-4-fluoro-1H-indol-2-yl)methyl)carbamate. LC-MS (ESI) m/z calcd for C₁₄H₁₆BrFN₂O₂: 342.04; found: 287.0 [M−tBu+H]⁺.

### Step 5: tert-butyl ((6-(5,5-dimethyl-1,3,2-dioxaborinan-2-yl)-4-fluoro-1H-indol-2-yl)methyl)carbamate

To a solution of tert-butyl ((6-bromo-4-fluoro-1H-indol-2-yl)methyl)carbamate (2.00 g, 5.83 mmol, 1.00 eq) in dioxane (20 mL) was added 2-(5,5-dimethyl-1,3,2-dioxaborinan-2-yl)-5,5-dimethyl-1,3,2-dioxaborinane (3.95 g, 17.4 mmol, 3.00 eq) and KOAc (1.26 g, 12.8 mmol, 2.20 eq). The reaction mixture was degassed and purged with N₂ three times before Pd(PPh₃)₂Cl₂ (818 mg, 1.17 mmol, 0.20 eq) was added. The reaction mixture was heated and stirred at 120° C. for 2 h. After cooling to room temperature, the reaction mixture was filtered, and the filtrate was concentrated under reduced pressure. Purification by column chromatography gave tert-butyl ((6-(5,5-dimethyl-1,3,2-dioxaborinan-2-yl)-4-fluoro-1H-indol-2-yl)methyl)carbamate.

### Step 6: tert-butyl ((4-fluoro-6-hydroxy-1H-indol-2-yl)methyl)carbamate

To a mixture of tert-butyl N-((6-(5,5-dimethyl-1,3,2-dioxaborinan-2-yl)-4-fluoro-1H-indol-2-yl)methyl)carbamate (2.10 g, 5.58 mmol, 1.00 eq) and NaOH (446 mg, 11.2 mmol, 2.00 eq) in THF (20 mL) was added H₂O₂ (6.33 g, 55.8 mmol, 30% purity, 10.0 eq) dropwise at 0° C. The reaction mixture was warmed to 25° C. and stirred for 16 h. The reaction mixture was quenched by sat. aq. Na₂S₂O₃ solution (30 mL), then extracted with ethyl acetate (2×30 mL). The combined organic phase was washed with brine (2×25 mL), dried over Na₂SO₄, filtered, and concentrated under reduced pressure. Purification by column chromatography gave tert-butyl N-((4-fluoro-6-hydroxy-1H-indol-2-yl)methyl)carbamate. LC-MS (ESI) m/z calcd for C₁₄H₁₇FN₂O₃: 280.12; found: 281.1 [M+H]⁺.

### Step 7: tert-butyl ((4-fluoro-6-(thiazol-4-ylmethoxy)-1H-indol-2-yl)methyl)carbamate

To a mixture of tert-butyl N-((4-fluoro-6-hydroxy-1H-indol-2-yl)methyl)carbamate (400 mg, 1.43 mmol, 1.00 eq), 4-(chloromethyl)thiazole (242 mg, 1.43 mmol, 1.00 eq, HCl) and DIEA (184 mg, 1.43 mmol, 1.00 eq) in DMF (5 mL) was added Cs₂CO₃ (930 mg, 2.85 mmol, 2.00 eq) and KI (118 mg, 713 μmol, 0.50 eq), the reaction mixture was heated and stirred at 70° C. for 1 h. After cooling to room temperature, the reaction mixture was poured into water (15 mL) and stirred for 5 min. The aqueous phase was extracted with ethyl acetate (2×10 mL). The combined organic phase was washed with brine (2×10 mL), dried over anhydrous Na₂SO₄, filtered, and concentrated under reduced pressure. Purification by column chromatography gave tert-butyl N-((4-fluoro-6-(thiazol-4-ylmethoxy)-1H-indol-2-yl)methyl)carbamate. LC-MS (ESI) m/z calcd for C₁₈H₂₀FN₃O₃S: 377.12; found: 378.1 [M+H]⁺.

### Step 8: (4-fluoro-6-(thiazol-4-ylmethoxy)-1H-indol-2-yl)methanamine hydrochloride

To a solution of tert-butyl N-((4-fluoro-6-(thiazol-4-ylmethoxy)-1H-indol-2-yl)methyl)carbamate (370 mg, 980 μmol, 1.00 eq) in EtOAc (0.5 mL) was added HCl/EtOAc (4 M in EtOAc, 10.5 mL, 43.0 eq) dropwise at 0° C. The reaction mixture was stirred at 25° C. for 1 h. The reaction mixture was concentrated under reduced pressure to give [4-fluoro-6-(thiazol-4-ylmethoxy)-1H-indol-2-yl]methanamine hydrochloride (300 mg), which was used directly without further purification. LC-MS (ESI) m/z calcd for C₁₃H₁₂FN₃OS: 277.07; found: 261.0 [M−NH₂]⁺.

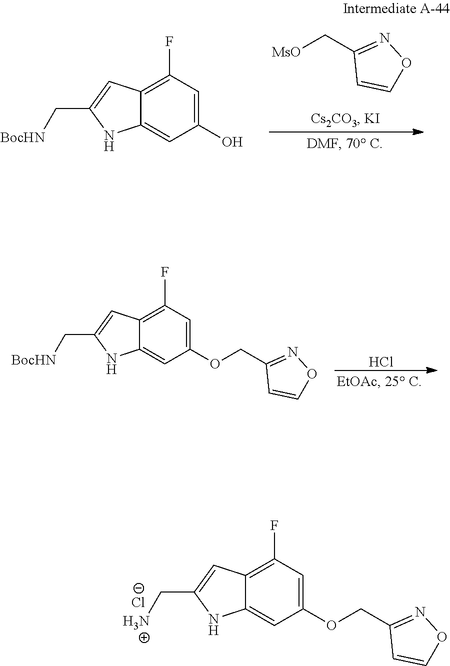 [SMILES: CC(C)(C)OC(=O)NCc1cc2c(F)cc(O)cc2[nH]1 ; CS(=O)(=O)OCc1ccon1 ; CC(C)(C)OC(=O)NCc1cc2c(F)cc(OCc3ccon3)cc2[nH]1 ; Cl ; [Cl-] ; [NH3+]Cc1cc2c(F)cc(OCc3ccon3)cc2[nH]1]

### Step 1: tert-butyl ((4-fluoro-6-(isoxazol-3-ylmethoxy)-1H-indol-2-yl)methyl)carbamate

To a mixture of tert-butyl ((4-fluoro-6-hydroxy-1H-indol-2-yl)methyl)carbamate (660 mg, 1.88 mmol, 1.00 eq) and isoxazol-3-ylmethyl methanesulfonate (400 mg, 2.26 mmol, 1.20 eq) in DMF (7 mL) was added KI (469 mg, 2.83 mmol, 1.50 eq) and Cs₂CO₃ (1.84 g, 5.65 mmol, 3.00 eq). The reaction mixture was warmed to 70° C. and stirred for 1 h. The reaction mixture was allowed to cool to room temperature and quenched with H₂O (15 mL). The aqueous phase was extracted with ethyl acetate (2×10 mL). The combined organic layers were washed with brine (20 mL), dried over anhydrous Na₂SO₄, filtered, and concentrated under reduced pressure. Purification by column chromatography gave tert-butyl ((4-fluoro-6-(isoxazol-3-ylmethoxy)-1H-indol-2-yl)methyl)carbamate. LC-MS (ESI) m/z calcd for C₁₈H₂₀FN₃O₄: 361.14; found: 362.2 [M+H]⁺.

### Step 2: (4-fluoro-6-(isoxazol-3-ylmethoxy)-1H-indol-2-yl)methanamine hydrochloride

To a solution of tert-butyl ((4-fluoro-6-(isoxazol-3-ylmethoxy)-1H-indol-2-yl)methyl)carbamate (380 mg, 1.05 mmol, 1.00 eq) in ethyl acetate (1 mL) was added HCl (4.00 M in EtOAc, 2 mL) dropwise. The reaction mixture was stirred at 25° C. for 1 h. Concentration of the reaction mixture under reduced pressure gave (4-fluoro-6-(isoxazol-3-ylmethoxy)-1H-indol-2-yl)methanamine hydrochloride, which was used without further purification. LC-MS (ESI) m/z calcd for C₁₃H₁₂FN₃O₂: 261.09; found: 245.0 [M−NH₂]⁺.

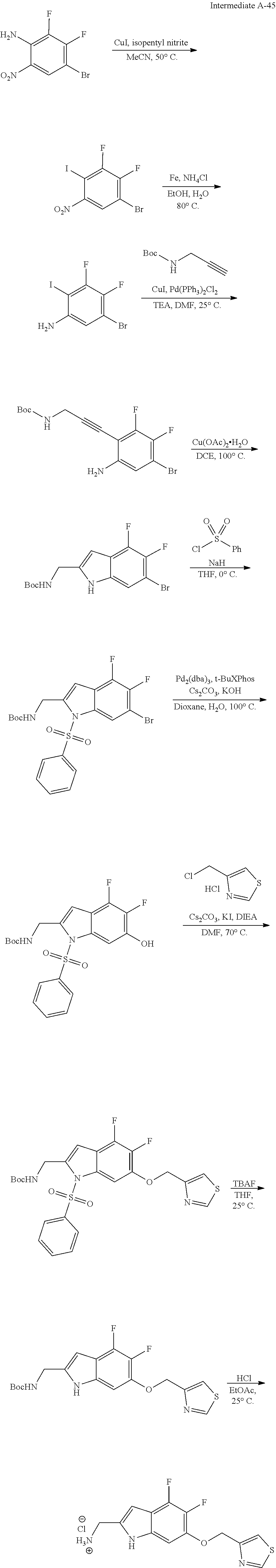

### Step 1: 1-bromo-2,3-difluoro-4-iodo-5-nitrobenzene

To a suspension of 4-bromo-2,3-difluoro-6-nitro-aniline (10.0 g, 39.5 mmol, 1.00 eq) in MeCN (100 mL) was added CuI (11.2 g, 59.2 mmol, 1.50 eq) and isopentyl nitrite (59.2 mmol, 7.98 mL, 1.50 eq). The reaction mixture was warmed to 50° C. and stirred under an atmosphere of N₂ for 16 h. The reaction mixture was allowed to cool to room temperature and was poured into water (100 mL) and stirred for 2 min. The aqueous phase was extracted with ethyl acetate (2×80 mL). The combined organic phase was washed with brine (2×80 mL), dried over anhydrous Na₂SO₄, filtered, and concentrated under reduced pressure. Purification by column chromatography gave 1-bromo-2,3-difluoro-4-iodo-5-nitrobenzene.

### Step 2: 5-bromo-3,4-difluoro-2-iodoaniline

To a solution of 1-bromo-2,3-difluoro-4-iodo-5-nitrobenzene (7.00 g, 19.2 mmol, 1.00 eq) in EtOH (70.0 mL) and H₂O (10.0 mL) was added NH₄Cl (4.12 g, 76.9 mmol, 4.00 eq) and the reaction mixture was warmed to 80° C. and stirred. Fe (10.7 g, 192 mmol, 10 eq) was added in portions and the reaction mixture was stirred at 80° C. for 1 h. The reaction mixture was allowed to cool to room temperature and was then filtered through Celite. The filter cake was washed with ethyl acetate (5×50 mL). The filtrate was concentrated under reduced pressure. Purification by column chromatography gave 5-bromo-3,4-difluoro-2-iodoaniline. LC-MS (ESI) m/z calcd for C₆H₃BrF₂IN: 332.85; found 333.8 [M+H]⁺.

### Step 3: tert-butyl (3-(6-amino-4-bromo-2,3-difluorophenyl)prop-2-yn-1-yl)carbamate

A mixture of 5-bromo-3,4-difluoro-2-iodoaniline (3.50 g, 10.4 mmol, 1.00 eq), tert-butyl N-prop-2-ynylcarbamate (2.44 g, 15.7 mmol, 1.50 eq) and TEA (52.4 mmol, 7.29 mL, 5.00 eq) in DMF (35.0 mL) was degassed and purged with N₂ three times, then CuI (399 mg, 2.10 mmol, 0.20 eq) and Pd(PPh₃)₂Cl₂ (735 mg, 1.05 mmol, 0.10 eq) were added. The reaction mixture was stirred at 25° C. for 16 h under an atmosphere of N₂. The reaction mixture was allowed to cool to room temperature and filtered through Celite. The filter cake was washed with ethyl acetate (5×50 mL). The filtrate was concentrated under reduced pressure. Purification by column chromatography gave tert-butyl (3-(6-amino-4-bromo-2,3-difluorophenyl)prop-2-yn-1-yl)carbamate. LC-MS (ESI) m/z calcd for C₁₄H₁₅BrF₂N₂O₂: 360.03; found 304.9 [M−tBu+2H]⁺.

### Step 4: tert-butyl ((6-bromo-4,5-difluoro-1H-indol-2-yl)methyl)carbamate

To a solution of tert-butyl (3-(6-amino-4-bromo-2,3-difluorophenyl)prop-2-yn-1-yl)carbamate (2.20 g, 6.09 mmol, 1.00 eq) in 1,1-dichloroethane (22.0 mL) was added Cu(OAc)₂·H₂O (608 mg, 3.05 mmol, 0.50 eq) in one portion. The reaction mixture was warmed to 100° C. and stirred for 6 h. The reaction mixture was allowed to cool to room temperature and was filtered through Celite. The filtrate was concentrated under reduced pressure. Purification by column chromatography gave tert-butyl ((6-bromo-4,5-difluoro-1H-indol-2-yl)methyl)carbamate. LC-MS (ESI) m/z calcd for C₁₄H₁₅BrF₂N₂O₂: 360.03; found 304.9 [M−tBu+2H]⁺.

### Step 5: tert-butyl ((6-bromo-4,5-difluoro-1-(phenylsulfonyl)-1H-indol-2-yl)methyl)carbamate

To a solution of tert-butyl ((6-bromo-4,5-difluoro-1H-indol-2-yl)methyl)carbamate (1.00 g, 2.77 mmol, 1.00 eq) in THF (1.00 mL) cooled to 0° C. was added NaH (221 mg, 5.54 mmol, 60% w/w, 2.00 eq) in portions. The reaction mixture was stirred at 0° C. for 0.5 h under an atmosphere of N₂. Benzenesulfonyl chloride (586 mg, 3.32 mmol, 1.20 eq) was added dropwise, and the reaction mixture was stirred at 0° C. for 1 h. The reaction mixture was cooled to 0° C., quenched by addition of water (20 mL), and stirred for 2 min. The aqueous phase was extracted with ethyl acetate (2×20 mL). The combined organic phase was washed with brine (2×20 mL), dried over anhydrous Na₂SO₄, filtered, and concentrated under reduced pressure. Purification by column chromatography gave tert-butyl ((6-bromo-4,5-difluoro-1-(phenylsulfonyl)-1H-indol-2-yl)methyl)carbamate. LC-MS (ESI) m/z calcd for C₂₀H₁₉BrF₂N₂O₄S: 500.02; found 523.0 [M+Na]⁺.

### Step 6: tert-butyl ((4,5-difluoro-6-hydroxy-1-(phenylsulfonyl)-1H-indol-2-yl) methyl) carbamate

To a mixture of tert-butyl ((6-bromo-4,5-difluoro-1-(phenylsulfonyl)-1H-indol-2-yl)methyl)carbamate (800 mg, 1.60 mmol, 1.00 eq) in dioxane (10 mL) and H₂O (2 mL) were added Cs₂CO₃ (1.04 g, 3.19 mmol, 2.00 eq) and KOH (358 mg, 6.38 mmol, 4.00 eq) in one portion, then the reaction mixture was degassed and purged with N₂ 3 times. Tris(dibenzylideneacetone)dipalladium (Pd₂(dba)₃, 146 mg, 159 μmol, 0.1 eq) and ditert-butyl-[2-(2,4,6-triisopropylphenyl)phenyl]phosphane (t-BuXPhos, 67.7 mg, 159 μmol, 0.1 eq) were added in one portion, and the reaction mixture was heated to 100° C. and stirred for 2 h. The reaction mixture was allowed to cool to room temperature and was then filtered through Celite. The filtrate was concentrated under reduced pressure. Purification by column chromatography gave tert-butyl ((4,5-difluoro-6-hydroxy-1-(phenylsulfonyl)-1H-indol-2-yl) methyl) carbamate. LC-MS (ESI) m/z calcd for C₂₀H₂₀F₂N₂O₅S: 438.11; found 461.1 [M+Na]⁺.

### Step 7: tert-butyl ((4,5-difluoro-1-(phenylsulfonyl)-6-(thiazol-4-ylmethoxy)-1H-indol-2-yl) methyl) carbamate

To a mixture of 4-(chloromethyl)thiazole hydrochloride (107 mg, 629 μmol, 1.20 eq, HCl salt) and tert-butyl ((4,5-difluoro-6-hydroxy-1-(phenylsulfonyl)-1H-indol-2-yl) methyl) carbamate (230 mg, 524 μmol, 1.00 eq) in DMF (2 mL) were added Cs₂CO₃ (170 mg, 524 μmol, 1.00 eq), DIEA (1.05 mmol, 182 μL, 2.00 eq) and KI (6.10 mg, 36.7 μmol, 0.07 eq) in one portion. The reaction mixture was heated to 70° C. and was stirred for 2 h. The reaction mixture was allowed to cool to room temperature before it was poured into water (10 mL) and stirred for 2 mins. The aqueous phase was extracted with ethyl acetate (2×10 mL). The combined organic phase was washed with brine (2×10 mL), dried over anhydrous Na₂SO₄, filtered, and concentrated under reduced pressure. Purification by prep-TLC gave tert-butyl ((4,5-difluoro-1-(phenylsulfonyl)-6-(thiazol-4-ylmethoxy)-1H-indol-2-yl) methyl) carbamate. LC-MS (ESI) m/z calcd for C₂₄H₂₃F₂N₃O₅S₂: 535.10; found 558.2 [M+Na]⁺.

### Step 8: tert-butyl ((4,5-difluoro-6-(thiazol-4-ylmethoxy)-1H-indol-2-yl)methyl)carbamate

To a solution of tert-butyl ((4,5-difluoro-1-(phenylsulfonyl)-6-(thiazol-4-ylmethoxy)-1H-indol-2-yl) methyl) carbamate (220 mg, 410 μmol, 1.00 eq) in THF (2 mL) was added TBAF (1 M in THF, 4.11 mL, 10.0 eq) dropwise. The reaction mixture was stirred at 25° C. for 1 h. The reaction mixture was poured into water (5 mL) and stirred for 2 mins. The aqueous phase was extracted with ethyl acetate (2×10 mL). The combined organic phase was washed with sat. aq. NH₄Cl solution (5×10 mL), dried over anhydrous Na₂SO₄, and filtered. Concentration under reduced pressure gave tert-butyl ((4,5-difluoro-6-(thiazol-4-ylmethoxy)-1H-indol-2-yl)methyl)carbamate, which was used without further purification. LC-MS (ESI) m/z calcd for C₁₈H₁₉F₂N₃O₃S: 395.11; found 396.1 [M+H]⁺.

### Step 9: (4,5-difluoro-6-(thiazol-4-ylmethoxy)-1H-indol-2-yl) methanamine hydrochloride

To a solution of tert-butyl ((4,5-difluoro-6-(thiazol-4-ylmethoxy)-1H-indol-2-yl)methyl)carbamate (150 mg, 379 μmol, 1.00 eq) in EtOAc (2 mL) was added HCl (4 M in EtOAc, 20 mL, 210 eq) dropwise. The reaction mixture was stirred at 25° C. for 1 h. Concentration of the reaction mixture under reduced pressure gave (4,5-difluoro-6-(thiazol-4-ylmethoxy)-1H-indol-2-yl) methanamine hydrochloride, which was used without further purification. LC-MS (ESI) m/z calcd for C₁₃H₁₁F₂N₃OS: 295.06; found 317.9 [M+Na]⁺.

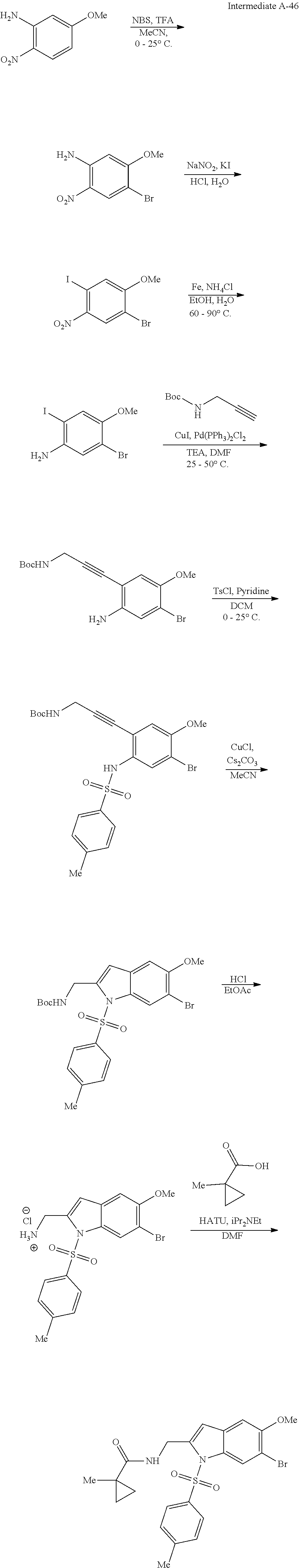

### Step 1: 4-bromo-5-methoxy-2-nitroaniline

To a solution of 5-methoxy-2-nitroaniline (6.20 g, 36.9 mmol, 1.00 eq) in MeCN (90 mL) were added NBS (6.56 g, 36.9 mmol, 1.00 eq) and TFA (4.20 g, 36.9 mmol, 1.00 eq) at 0° C. and the reaction mixture was warmed to 25° C. After 16 h, water (100 mL) was added, and the mixture was extracted with EtOAc (3×30 mL). The combined organic layers were washed with water (3×30 mL), dried over anhydrous Na₂SO₄, filtered, concentrated under reduced pressure. Purification by column chromatography gave 4-bromo-5-methoxy-2-nitroaniline. LC-MS (ESI) m/z calcd for C₇H₇BrN₂O₃: 245.96; found: 246.9 [M+H]⁺.

### Step 2: 5-bromo-6-methoxy-1H-indole-2-carboxylic acid

To a solution of 4-bromo-5-methoxy-2-nitroaniline (7.15 g, 28.9 mmol, 1.00 eq) in HCl (12 M aqueous, 73.0 mL, 30.2 eq) were added NaNO₂ (2.60 g, 37.6 mmol, 1.30 eq) and H₂O (37.5 mL) at 0° C. A solution of KI (14.4 g, 86.8 mmol, 3.00 eq) in H₂O (10 mL) was then added dropwise and the reaction mixture was warmed to 25° C. After 6 h, water (120 mL) was added, and was adjusted to pH=8 by addition of aqueous NaOH (4 M). The resulting mixture was extracted with EtOAc (3×60 mL). The combined organic layers were washed with water (3×60 mL), dried over anhydrous Na₂SO₄, filtered, and concentrated under reduced pressure. Purification by silica gel chromatography gave 1-bromo-4-iodo-2-methoxy-5-nitrobenzene.

### Step 3: 5-bromo-2-iodo-4-methoxyaniline

To a solution of 1-bromo-4-iodo-2-methoxy-5-nitrobenzene (8.40 g, 23.5 mmol, 1.00 eq) in EtOH (100 mL) and H₂O (25 mL) were added Fe (3.93 g, 70.4 mmol, 3.00 eq) and NH₄Cl (6.28 g, 117 mmol, 5.00 eq) at 60° C. The reaction mixture was then heated to 90° C. for 1 h and cooled to room temperature. Water (100 mL) was then added, and the mixture was extracted with EtOAc (3×50 mL). The combined organic layers were washed with water (3×50 mL), dried over anhydrous Na₂SO₄, filtered, and concentrated under reduced pressure. Purification by column chromatography gave 5-bromo-2-iodo-4-methoxyaniline. LC-MS (ESI) m/z calcd for C₇H₇BrINO: 326.88; found: 327.9 [M+H]⁺.

### Step 4: tert-butyl (3-(2-amino-4-bromo-5-methoxyphenyl)prop-2-yn-1-yl)carbamate

To a solution of 5-bromo-2-iodo-4-methoxyaniline (8.49 g, 25.9 mmol, 1.00 eq) and tert-butyl N-prop-2-ynylcarbamate (6.03 g, 38.9 mmol, 1.50 eq) in DMF (250 mL) were added CuI (0.99 g, 5.2 mmol. 0.2 eq), TEA (13.1 g, 130 mmol, 5 eq) and Pd(PPh₃)₂Cl₂ (1.82 g, 2.59 mmol, 0.10 eq). The reaction mixture was heated to 50° C. for 2 h. The reaction mixture was then cooled to room temperature and quenched with water (200 mL). The mixture was then extracted with EtOAc (3×500 mL). The combined organic layers were washed with water (3×50 mL), dried over anhydrous Na₂SO₄, filtered, and concentrated under reduced pressure. Purification by silica gel chromatography gave tert-butyl (3-(2-amino-4-bromo-5-methoxyphenyl)prop-2-yn-1-yl)carbamate. LC-MS (ESI) m/z calcd for C₁₅H₁₉BrN₂O₃: 354.06; found: 299.1 [M−tBu+H]⁺.

### Step 5: tert-butyl (3-(4-bromo-5-methoxy-2-((4-methylphenyl)sulfonamido)phenyl)prop-2-yn-1-yl)carbamate

To a mixture of tert-butyl (3-(2-amino-4-bromo-5-methoxyphenyl)prop-2-yn-1-yl)carbamate (7.25 g, 20.4 mmol, 1.00 eq) and pyridine (3.23 g, 40.8 mmol, 2.00 eq) in DCM (75 mL) was added 4-methylbenzenesulfonyl chloride (4.28 g, 22.5 mmol, 1.10 eq) at 0° C. The reaction mixture was warmed to room temperature. After 16 h, the reaction mixture was quenched water (100 mL) and the resulting mixture was extracted with EtOAc (3×30 mL). The combined organic layers were washed with water (3×30 mL), dried over anhydrous Na₂SO₄, filtered, and concentrated under reduced pressure. Purification by column chromatography gave tert-butyl (3-(4-bromo-5-methoxy-2-((4-methylphenyl)sulfonamido)phenyl)prop-2-yn-1-yl)carbamate.

### Step 6: tert-butyl ((6-bromo-5-methoxy-1-tosyl-1H-indol-2-yl)methyl)carbamate

To a solution of tert-butyl (3-(4-bromo-5-methoxy-2-((4-methylphenyl) sulfonamido) phenyl) prop-2-yn-1-yl) carbamate (13.0 g, 25.5 mmol, 1.00 eq) in MeCN (130 mL) were added CuCl (0.38 g, 3.83 mmol, 0.15 eq) and Cs₂CO₃ (1.25 g, 3.83 mmol, 0.15 eq). The reaction mixture was sparged with nitrogen and stirred at room temperature and stirred for 3 h. The reaction mixture was quenched with the addition of water (400 mL) and the resulting mixture was extracted with EtOAc (3×200 mL). The combined organic layers were washed with brine (200 mL), dried over anhydrous Na₂SO₄, filtered, and concentrated under reduced pressure. Purification by column chromatography gave tert-butyl ((6-bromo-5-methoxy-1-tosyl-1H-indol-2-yl)methyl)carbamate. LC-MS (ESI) m/z calcd for C₂₂H₂₅BrN₂O₅S: 508.07; found: 453.1 [M−tBu+H]⁺.

### Step 7: (6-bromo-5-methoxy-1-tosyl-1H-indol-2-yl)methanamine

To a solution of tert-butyl ((6-bromo-5-methoxy-1-tosyl-1H-indol-2-yl)methyl)carbamate (5.8 g, 11.3 mmol, 1.00 eq) in EtOAc was added HCl (4 M in EtOAc, 50 mL, 17.5 eq). The reaction was stirred at 25° C. for 1 h and concentrated under reduced pressure to give (6-bromo-5-methoxy-1-tosyl-1H-indol-2-yl)methanamine hydrochloride. LC-MS (ESI) m/z calcd for C₁₇H₁₇BrN₂O₃S: 408.1; found: 391.9 [M−NH₂+H]⁺.

### Step 8: N-((6-bromo-5-methoxy-1-tosyl-1H-indol-2-yl)methyl)-1-methylcyclopropane-1-carboxamide

To a solution of 1-methylcyclopropanecarboxylic acid (1.37 g, 13.6 mmol, 1.20 eq) in DMF (50 mL) was added HATU (6.50 g, 17.1 mmol, 1.50 eq). The reaction mixture was stirred for 30 min and DIPEA (5.89 g, 45.5 mmol, 4.00 eq) and (6-bromo-5-methoxy-1-tosyl-1H-indol-2-yl)methanamine hydrochloride (5.08 g, 11.4 mmol, 1.00 eq) were added and the resulting reaction mixture was stirred for 1 h. The reaction mixture was quenched with water (40 mL) and the mixture was extracted with EtOAc (3×50 mL). The combined organic layers were washed with brine (50 mL), dried over anhydrous Na₂SO₄, filtered, and concentrated under reduced pressure. Purification by column chromatography gave N-((6-bromo-5-methoxy-1-tosyl-1H-indol-2-yl)methyl)-1-methylcyclopropane-1-carboxamide. LC-MS (ESI) m/z calcd for C₂₂H₂₃BrN₂O₄S: 490.06; found: 493.1 [M+H]⁺.

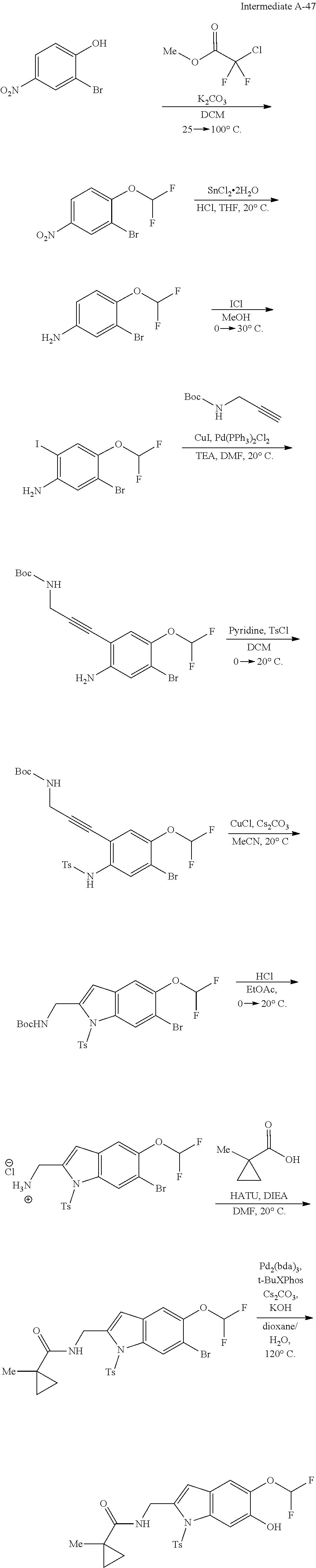

### Step 1: 2-bromo-1-(difluoromethoxy)-4-nitrobenzene

To a solution of 2-bromo-4-nitrophenol (7.70 g, 35.3 mmol, 1.00 eq) in DMF (100 mL) was added K₂CO₃ (4.88 g, 35.3 mmol, 1.00 eq). The resulting reaction mixture was stirred at 25° C. After 15 min, methyl 2-chloro-2,2-difluoro-acetate (6.64 g, 45.9 mmol, 1.30 eq) was added and the reaction mixture was heated to 100° C. and for 1 h. After cooling to room temperature, the reaction mixture was quenched with water (150 mL) and adjusted to pH 7 with 1 N HCl (50 mL), the aqueous phase was extracted with ethyl acetate (2×80 mL). The combined organic phase was washed with brine (50 mL), dried over Na₂SO₄, filtered, and concentrated under reduced pressure. Purification by column chromatography gave 2-bromo-1-(difluoromethoxy)-4-nitrobenzene.

### Step 2: 3-bromo-4-(difluoromethoxy)aniline

A mixture of 2-bromo-1-(difluoromethoxy)-4-nitrobenzene (6.00 g, 22.3 mmol, 1.00 eq) and SnCl₂·2H₂O (25.2 g, 112 mmol, 5.00 eq) in THF (10 mL) was added to HCl (12 M in H₂O, 60 mL, 32.2 eq) in portions. Then the reaction mixture was stirred at 20° C. for 2 h. The reaction mixture was poured into water (50 mL), slowly and adjusted to pH 7 with sat. aq. NaHCO₃ solution, then the aqueous phase extracted with ethyl acetate (3×50 mL). The combined organic phase was washed with brine (50 mL), dried over Na₂SO₄, filtered, and concentrated under reduced pressure. Purification by column chromatography gave 3-bromo-4-(difluoromethoxy) aniline. LC-MS (ESI) m/z calcd for C₇H₆BrF₂NO: 236.96; found: 238.0 [M+H]⁺.

### Step 3: 5-bromo-4-(difluoromethoxy)-2-iodoaniline

To a solution of 3-bromo-4-(difluoromethoxy) aniline (8.00 g, 33.6 mmol, 1.00 eq) in MeOH (80 mL) was added ICl (6.00 g, 36.9 mmol, 1.89 mL, 1.10 eq) in portions at 0° C. Then the reaction mixture was heated to 30° C. for 2 h. After cooling to room temperature, the reaction mixture was poured into water (60 mL) and stirred for 2 min. The aqueous phase was extracted with ethyl acetate (3×100 mL). The combined organic phase was washed with brine (2×100 mL), dried over anhydrous Na₂SO₄, filtered, and concentrated under reduced pressure to give a residue. Purification by column chromatography to give 5-bromo-4-(difluoromethoxy)-2-iodoaniline. LC-MS (ESI) m/z calcd for C₇H₅BrF₂INO: 362.86; found: 363.9 [M+H]⁺.

### Step 4: tert-butyl (3-(2-amino-4-bromo-5-(difluoromethoxy)phenyl)prop-2-yn-1-yl)carbamate

To a mixture of tert-butyl prop-2-yn-1-ylcarbamate (1.28 g, 8.24 mmol, 1.00 eq) and 5-bromo-4-(difluoromethoxy)-2-iodoaniline (3.00 g, 8.24 mmol, 1.00 eq) in DMF (10 mL) was added TEA (41.2 mmol, 5.74 mL, 5.00 eq). The reaction mixture was degassed and purged with N₂ three times, then CuI (157.00 mg, 824 μmol, 0.1 eq) and Pd(PPh₃)₂Cl₂ (174 mg, 247 μmol, 0.03 eq) were added under N₂ atmosphere. The reaction mixture was stirred at 20° C. for 1 h before being poured into water (20 mL) and stirred for 5 min. The aqueous phase was extracted with ethyl acetate (3×30 mL). The combined organic phase was washed with brine (2×20 mL), dried over anhydrous Na₂SO₄, filtered, and concentrated under reduced pressure. Purification by column chromatography gave tert-butyl (3-(2-amino-4-bromo-5-(difluoromethoxy)phenyl)prop-2-yn-1-yl)carbamate. LC-MS (ESI) m/z calcd for C₁₈H₁₇BrF₂N₂O: 390.04; found: 335.0 [M−t-Bu+H]⁺.

### Step 5: tert-butyl (3-(4-bromo-5-(difluoromethoxy)-2-((4-methylphenyl)sulfonamido)phenyl)prop-2-yn-1-yl)carbamate

A mixture of tert-butyl (3-(2-amino-4-bromo-5-(difluoromethoxy)phenyl)prop-2-yn-1-yl)carbamate (2.70 g, 6.90 mmol, 1.00 eq) and pyridine (13.8 mmol, 1.11 mL, 2.00 eq) in DCM (27 mL) was brought to 0° C. before 4-methylbenzenesulfonyl chloride (1.45 g, 7.59 mmol, 1.10 eq) was added in portions. The reaction was then allowed to warm to 20° C. and stirred for 12 h. The reaction mixture was quenched with H₂O (30 ml) at 25° C., and the aqueous phase was extracted with ethyl acetate (3×30 mL). The combined organic phase was washed with brine (30 mL), dried over Na₂SO₄, filtered, and concentrated under reduced pressure. Purification by column chromatography gave tert-butyl (3-(4-bromo-5-(difluoromethoxy)-2-((4-methylphenyl) sulfonamido) phenyl) prop-2-yn-1-yl) carbamate (3.60 g). LC-MS (ESI) m/z calcd for C₂₂H₂₃BrF₂N₂O₅S: 544.05; found: 445.0 [M−Boc+H]⁺.

### Step 6: tert-butyl ((6-bromo-5-(difluoromethoxy)-1-tosyl-1H-indol-2-yl)methyl)carbamate

To a solution of tert-butyl (3-(4-bromo-5-(difluoromethoxy)-2-((4-methylphenyl) sulfonamido) phenyl) prop-2-yn-1-yl) carbamate (3.60 g, 6.60 mmol, 1.00 eq) in MeCN (36 mL) were added Cs₂CO₃ (323 mg, 990 μmol, 0.150 eq) and CuCl (98.0 mg, 990 μmol, 0.15 eq) in one portion under N₂ atmosphere. The reaction mixture was degassed and purged with N₂ three times and then stirred at 20° C. for 12 h under N₂ atmosphere. The reaction mixture was poured into water (20 mL) and stirred for 2 min. The aqueous phase was extracted with ethyl acetate (3×25 mL). The combined organic phase was washed with brine (2×20 mL), dried over anhydrous Na₂SO₄, filtered, and concentrated under reduced pressure. Purification by column chromatography gave tert-butyl ((6-bromo-5-(difluoromethoxy)-1-tosyl-1H-indol-2-yl) methyl) carbamate. LC-MS (ESI) m/z calcd for C₂₂H₂₃BrF₂N₂O₅S: 544.05; found: 428.1 [M−NH₂Boc+H]⁺.

### Step 7: tert-butyl ((6-bromo-5-(difluoromethoxy)-1-tosyl-1H-indol-2-yl)methyl)carbamate hydrochloride

To a solution of tert-butyl ((6-bromo-5-(difluoromethoxy)-1-tosyl-1H-indol-2-yl) methyl) carbamate (2.80 g, 5.13 mmol, 1.00 eq) in EtOAc (8 mL) was added HCl/EtOAc (4 M in EtOAc, 30 mL, 23.3 eq) at 0° C. dropwise. After the addition, the reaction was allowed to warm to 20° C. and stirred for 0.5 h. The reaction mixture was concentrated under reduced pressure to give (6-bromo-5-(difluoromethoxy)-1-tosyl-1H-indol-2-yl) methanamine hydrochloride, which was used without of further purification. LC-MS (ESI) m/z calcd for C₂₂H₂₃BrF₂N₂O₅S: 544.05; found: 428.0 [M−NH₂]⁺.

### Step 8: N-((6-bromo-5-(difluoromethoxy)-1-tosyl-1H-indol-2-yl)methyl)-1-methylcyclopropane-1-carboxamide

To a solution of 1-methylcyclopropane-1-carboxylic acid (616 mg, 6.15 mmol, 1.20 eq) in DMF (20 mL) was added HATU (2.92 g, 7.69 mmol, 1.50 eq). The reaction mixture was stirred at 25° C. for 0.5 h. Then, DIEA (20.5 mmol, 3.57 mL, 4.00 eq) and (6-bromo-5-(difluoromethoxy)-1-tosyl-1H-indol-2-yl) methanamine hydrochloride (2.47 g, 5.13 mmol, 1.00 eq) were added in portions. The resulting reaction mixture was stirred at 20° C. for 1 h. The reaction mixture was quenched by addition of H₂O (30 mL), and the aqueous phase was extracted with ethyl acetate (3×20 mL). The combined organic phase was washed with brine (20 mL), dried over Na₂SO₄, filtered, and concentrated under reduced pressure. The resulting residue was purified by column chromatography to give N-((6-bromo-5-(difluoromethoxy)-1-tosyl-1H-indol-2-yl) methyl)-1-methylcyclopropane-1-carboxamide. LC-MS (ESI) m/z calcd for C₂₂H₂₁BrF₂N₂O₄S: 526.04; found: 527.2 [M+H]⁺.

### Step 9: N-((5-(difluoromethoxy)-6-hydroxy-1-tosyl-1H-indol-2-yl)methyl)-1-methylcyclopropane-1-carboxamide

To a solution of N-((6-bromo-5-(difluoromethoxy)-1-tosyl-1H-indol-2-yl) methyl)-1-methylcyclopropane-1-carboxamide (1.30 g, 2.47 mmol, 1.00 eq) in dioxane (12 mL) and H₂O (3 mL) was added Cs₂CO₃ (1.61 g, 4.93 mmol, 2.00 eq) and KOH (553 mg, 9.86 mmol, 4.00 eq) in one portion. The resulting reaction mixture was degassed and purged with N₂ three times before Pd₂(dba)₃ (226 mg, 247 μmol, 0.1 eq) and t-BuXPhos (105 mg, 247 μmol, 0.1 eq) were added. The reaction mixture was then heated to 120° C. and for 1 h under N₂ atmosphere. After cooling to room temperature, the reaction mixture was poured into H₂O (20 mL). The aqueous phase was extracted with ethyl acetate (3×20 mL). The combined organic phase was washed with brine (20 mL), dried over anhydrous Na₂SO₄, filtered, and concentrated under reduced pressure. The resulting residue was purified by column chromatography to give N-((5-(difluoromethoxy)-6-hydroxy-1-tosyl-1H-indol-2-yl)methyl)-1-methylcyclopropane-1-carboxamide. LC-MS (ESI) m/z calcd for C₂₂H₂₂F₂N₂O₅S: 464.12; found: 465.2 [M+H]⁺.

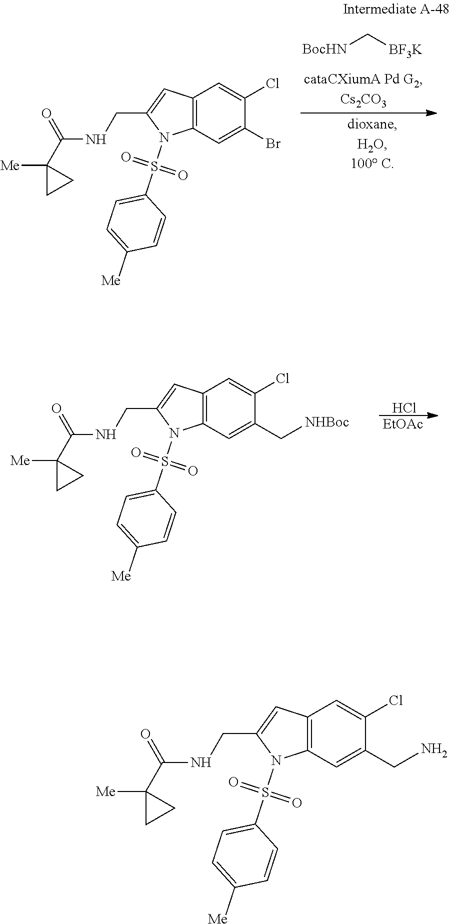

### Step 1: tert-butyl ((5-chloro-2-((1-methylcyclopropane-1-carboxamido)methyl)-1-tosyl-1H-indol-6-yl)methyl)carbamate

To a mixture of N-((6-bromo-5-chloro-1-tosyl-1H-indol-2-yl)methyl)-1-methylcyclopropane-1-carboxamide (3.00 g, 6.05 mmol, 1.00 eq) and tert-butyl ((trifluoro-λ⁴-boraneyl)methyl)carbamate, potassium salt (2.20 g, 9.08 mmol, 1.50 eq) in dioxane (16 mL) and water (4 mL), was added Cs₂CO₃ (4.00 g, 12.1 mmol, 2.00 eq). The mixture was degassed and purged with N₂ three times. CataCXiumA Pd G2 (405 mg, 605 μmol, 0.10 eq) was added and the resulting reaction mixture was heated to 100° C. for 16 h. The reaction mixture was cooled to 25° C. and quenched with water (15 mL) and filtered. The filtrate was extracted with EtOAc (3×30 mL). The combined organic layers were washed with brine (15 mL), dried over anhydrous Na₂SO₄, filtered, and concentrated under reduced pressure. The resulting crude residue was triturated with MTBE (20 mL) at 25° C. for 10 min to tert-butyl ((5-chloro-2-((1-methylcyclopropane-1-carboxamido)methyl)-1-tosyl-1H-indol-6-yl)methyl)carbamate. LC-MS (ESI) m/z calcd for C₂₇H₃₂ClN₃O₅S: 545.18; found: 490.1 [M−tBu+H]⁺.

### Step 2: N-((6-(aminomethyl)-5-chloro-1-tosyl-1H-indol-2-yl)methyl)-1-methylcyclopropane-1-carboxamide

To a solution of tert-butyl ((5-chloro-2-((1-methylcyclopropane-1-carboxamido)methyl)-1-tosyl-1H-indol-6-yl)methyl)carbamate (2.80 g, 5.13 mmol, 1.00 eq) in EtOAc (3 mL) was added HCl (4 M in EtOAc, 10 mL). The reaction mixture was stirred at 25° C. for 1 h and the reaction mixture was then quenched with the addition of water (15 mL) and adjusted to pH 7 by the addition of aqueous NaOH (4 M). The resulting mixture was extracted with EtOAc (3×30 mL). The combined organic layers were washed with brine (20 mL), dried over anhydrous Na₂SO₄, filtered, and concentrated under reduced pressure to give N-((6-(aminomethyl)-5-chloro-1-tosyl-1H-indol-2-yl)methyl)-1-methylcyclopropane-1-carboxamide. LC-MS (ESI) m/z calcd for C₂₂H₂₄ClN₃O₃S: 445.12; found: 429.1 [M−NH₂]⁺.

The following compounds in Table B-48 were synthesized using procedures similar to Intermediate A-48 using the appropriate starting materials.

TABLE B-48

| Intermediate | | | Exact mass | LCMS, |
|---|---|---|---|---|
| No. | Structure | IUPAC name | (g/mol) | found [M]⁺ |
| B-48-1 | 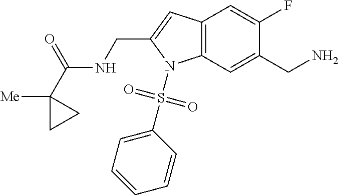 [SMILES: CC1(C(=O)NCc2cc3cc(F)c(CN)cc3n2S(=O)(=O)c2ccccc2)CC1] | N-((6- (aminomethyl)-5- fluoro-1- (phenylsulfonyl)- 1H-indol-2- yl)methyl)-1- methylcyclopropane- 1-carboxamide | 415.14 | 399.2 [M − NH₂ + H]⁺ |

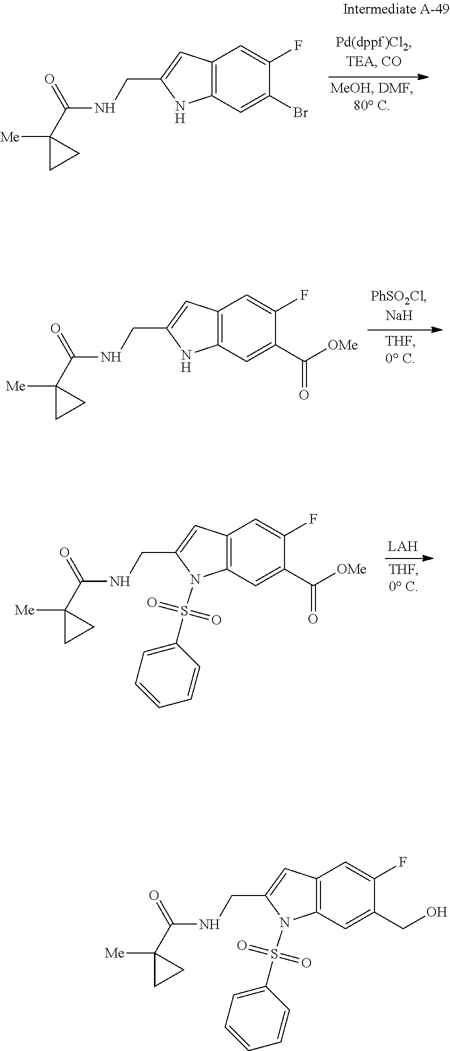 [SMILES: CC1(C(=O)NCc2cc3cc(F)c(Br)cc3[nH]2)CC1 ; COC(=O)c1cc2[nH]c(CNC(=O)C3(C)CC3)cc2cc1F ; COC(=O)c1cc2c(cc1F)cc(CNC(=O)C1(C)CC1)n2S(=O)(=O)c1ccccc1 ; CC1(C(=O)NCc2cc3cc(F)c(CO)cc3n2S(=O)(=O)c2ccccc2)CC1]

### Step 1: methyl 5-fluoro-2-((1-methylcyclopropane-1-carboxamido)methyl)-1H-indole-6-carboxylate

To a solution of N-((6-bromo-5-fluoro-1H-indol-2-yl)methyl)-1-methylcyclopropane-1-carboxamide (3.50 g, 10.7 mmol, 1.00 eq) in MeOH (40 mL) and DMF (8 mL) was added TEA (26.9 mmol, 3.75 mL, 2.50 eq) and (Pd(dppf)Cl₂·CH₂Cl₂ (878 mg, 1.08 mmol, 0.10 eq). The suspension was degassed and purged with CO three times. The reaction mixture was heated to 80° C. After 16 h at 30 Psi CO, the reaction mixture was diluted with H₂O (100 mL) at 25° C. The solution was extracted with ethyl acetate (2×70 mL). The combined organic layers were washed with brine (2×100 mL), dried over anhydrous Na₂SO₄, filtered, and concentrated under reduced pressure. Purification by column chromatography gave methyl 5-fluoro-2-((1-methylcyclopropane-1-carboxamido)methyl)-1H-indole-6-carboxylate. LC-MS (ESI) m/z calcd for C₁₆H₁₇FN₂O₃: 304.1; found 305.1 [M+H]⁺.

### Step 2: methyl 5-fluoro-2-((1-methylcyclopropane-1-carboxamido)methyl)-1-(phenylsulfonyl)-1H-indole-6-carboxylate

To a solution of NaH (157 mg, 3.94 mmol, 2.40 eq) in THF (4 mL) at 0° C. was added a solution of methyl 5-fluoro-2-((1-methylcyclopropane-1-carboxamido)methyl)-1H-indole-6-carboxylate (500 mg, 1.64 mmol, 1.00 eq) in THF (1 mL) dropwise. After 30 min at 0° C. benzenesulfonyl chloride (2.14 mmol, 273 μL, 1.30 eq) was added dropwise. After 2.5 h at 0° C., the reaction was quenched by addition of saturated aqueous NH₄Cl (10 mL) slowly at 0° C. The solution was extracted with ethyl acetate (3×10 mL). The combined organic layers were washed with brine (10 mL), dried over anhydrous Na₂SO₄, filtered, and concentrated under reduced pressure. Purification by column chromatography gave methyl 5-fluoro-2-((1-methylcyclopropane-1-carboxamido)methyl)-1-(phenylsulfonyl)-1H-indole-6-carboxylate. LC-MS (ESI) m/z calcd for C₂₂H₂₁FN₂O₅S: 444.1; found 445.2 [M+H]⁺.

### Step 3: N-((5-fluoro-6-(hydroxymethyl)-1-(phenylsulfonyl)-1H-indol-2-yl)methyl)-1-methylcyclopropane-1-carboxamide

To a solution of methyl 5-fluoro-2-((1-methylcyclopropane-1-carboxamido)methyl)-1-(phenylsulfonyl)-1H-indole-6-carboxylate (300 mg, 674 μmol, 1.00 eq) in THF (2 mL) at 0° C. was added LAH (76.0 mg, 2.02 mmol, 3.00 eq) in portions at 0° C. under N₂ atmosphere. After 2 h at 0° C., the reaction was quenched by successive dropwise addition of H₂O (0.077 mL), aqueous NaOH solution (15%), and H₂O (0.2 mL). After 15 minutes at 0° C., the reaction mixture was filtered. The filtrate was dried over anhydrous Na₂SO₄, filtered, and concentrated under reduced pressure to give N-((5-fluoro-6-(hydroxymethyl)-1-(phenylsulfonyl)-1H-indol-2-yl)methyl)-1-methylcyclopropane-1-carboxamide (200 mg), which was used directly without further purification. LC-MS (ESI) m/z calcd for C₂₁H₂₁FN₂O₄S: 416.1; found 417.2 [M+H]⁺.

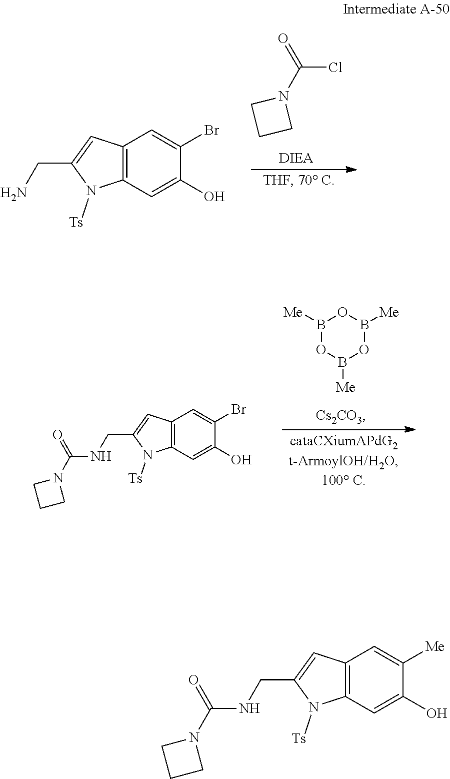 [SMILES: Cc1ccc(S(=O)(=O)n2c(CN)cc3cc(Br)c(O)cc32)cc1 ; O=C(Cl)N1CCC1 ; CB1OB(C)OB(C)O1 ; Cc1ccc(S(=O)(=O)n2c(CNC(=O)N3CCC3)cc3cc(Br)c(O)cc32)cc1 ; Cc1ccc(S(=O)(=O)n2c(CNC(=O)N3CCC3)cc3cc(C)c(O)cc32)cc1]

### Step 1: N-((5-bromo-6-hydroxy-1-tosyl-1H-indol-2-yl)methyl)azetidine-1-carboxamide

To a solution of 2-(aminomethyl)-5-bromo-1-tosyl-1H-indol-6-ol (3.00 g, 6.30 mmol, 1.00 eq, HBr salt) in THF (30 mL) was added DIEA (25.2 mmol, 4.39 mL, 4.00 eq) dropwise at 25° C., followed by addition of azetidine-1-carbonyl chloride (979 mg, 8.19 mmol, 1.30 eq). The resulting reaction mixture was heated and stirred at 70° C. for 1 h. After cooling to room temperature, the reaction mixture was poured into H₂O (5 mL), extracted with ethyl acetate (3×10 mL). The combined organic phase was washed with brine (2×10 mL), dried over anhydrous Na₂SO₄, filtered, and concentrated under reduced pressure. The resulting residue was triturated with MTBE (30 mL) at 30° C. for 15 min. The residue was filtered and the filter cake was collected to give N-((5-bromo-6-hydroxy-1-tosyl-1H-indol-2-yl)methyl)azetidine-1-carboxamide. LC-MS (ESI) m/z calcd for C₂₀H₂₀BrN₃O₄S: 477.04; found: 478.1 [M+H]⁺.

### Step 2: N-((6-hydroxy-5-methyl-1-tosyl-1H-indol-2-yl)methyl)azetidine-1-carboxamide

To a mixture of 2,4,6-trimethyl-1,3,5,2,4,6-trioxatriborinane (18.8 mmol, 2.63 mL, 5.00 eq) and N-((5-bromo-6-hydroxy-1-tosyl-1H-indol-2-yl)methyl)azetidine-1-carboxamide (1.80 g, 3.76 mmol, 1.00 eq) in H₂O (4 mL) and 2-methylbutan-2-ol (20 mL) was added cataCXium A Pd G2 (252 mg, 376 μmol, 0.10 eq) and Cs₂CO₃ (2.45 g, 7.53 mmol, 2.00 eq). The system was degassed under vacuum and purged with N₂ three times, and then the reaction mixture was heated and stirred at 100° C. for 12 h. After cooling to room temperature, the reaction mixture was poured into water (10 mL) and stirred for 2 min. The aqueous phase was extracted with ethyl acetate (3×20 mL). The combined organic phase was washed with brine (2×10 mL), dried over anhydrous Na₂SO₄, filtered, and concentrated under reduced pressure. Purification by column chromatography gave N-((6-hydroxy-5-methyl-1-tosyl-1H-indol-2-yl)methyl)azetidine-1-carboxamide. LC-MS (ESI) m/z calcd for C₂₁H₂₃N₃O₄S: 413.14; found: 414.2 [M+H]⁺.

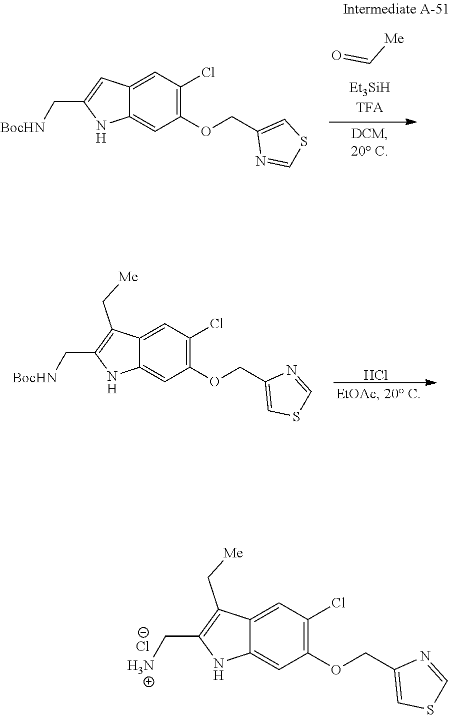

### Step 1: tert-butyl ((5-chloro-3-ethyl-6-(thiazol-4-ylmethoxy)-1H-indol-2-yl)methyl)carbamate

To a solution of Et₃SiH (159 mg, 1.37 mmol, 218 μL, 3.00 eq) in dichloromethane (1 mL) was added trifluoroacetic acid (78.1 mg, 685 μmol, 1.50 eq) in one portion at 20° C., the reaction mixture was degassed and purged with N₂ three times. The reaction mixture was stirred at 20° C. for 30 mins, then tert-butyl ((5-chloro-6-(thiazol-4-ylmethoxy)-1H-indol-2-yl) methyl) carbamate (180 mg, 456 μmol, 1.00 eq) and acetaldehyde (5 M in THF, 137.10 μL, 1.50 eq) were added in portions. The reaction mixture was stirred for 2 h at 20° C. The reaction mixture was cooled to 0° C., adjusted to pH 10 with 4 M aq. NaOH, then warmed to 20° C. and stirred for 10 mins. The aqueous phase was extracted with ethyl acetate (3×15 mL). The combined organic layers were dried over anhydrous Na₂SO₄, filtered and concentrated under reduced pressure to give a residue. The residue was purified by prep-TLC to give tert-butyl ((5-chloro-3-ethyl-6-(thiazol-4-ylmethoxy)-1H-indol-2-yl) methyl) carbamate. LC-MS (ESI) m/z calcd for C₂₀H₂₄ClN₃O₃S: 421.12; found: 422.2 [M+H]⁺.

### Step 2: (5-chloro-3-ethyl-6-(thiazol-4-ylmethoxy)-1H-indol-2-yl) methanamine hydrochloride

To a solution of tert-butyl ((5-chloro-3-ethyl-6-(thiazol-4-ylmethoxy)-1H-indol-2-yl) methyl) carbamate (100 mg, 237 μmol, 1.00 eq) in ethyl acetate (1 mL) was added HCl (4 M in EtOAc, 59.2 μL, 1.00 eq) in portions at 20° C. The reaction mixture was stirred at 20° C. for 2 h. The reaction mixture was concentrated under reduced pressure to give (5-chloro-3-ethyl-6-(thiazol-4-ylmethoxy)-1H-indol-2-yl) methanamine hydrochloride, which was used for next step directly without further purification. LC-MS (ESI) m/z calcd for C₁₅H₁₆ClN₃OS: 321.07; found: 305.1 [M−NH₂]⁺.

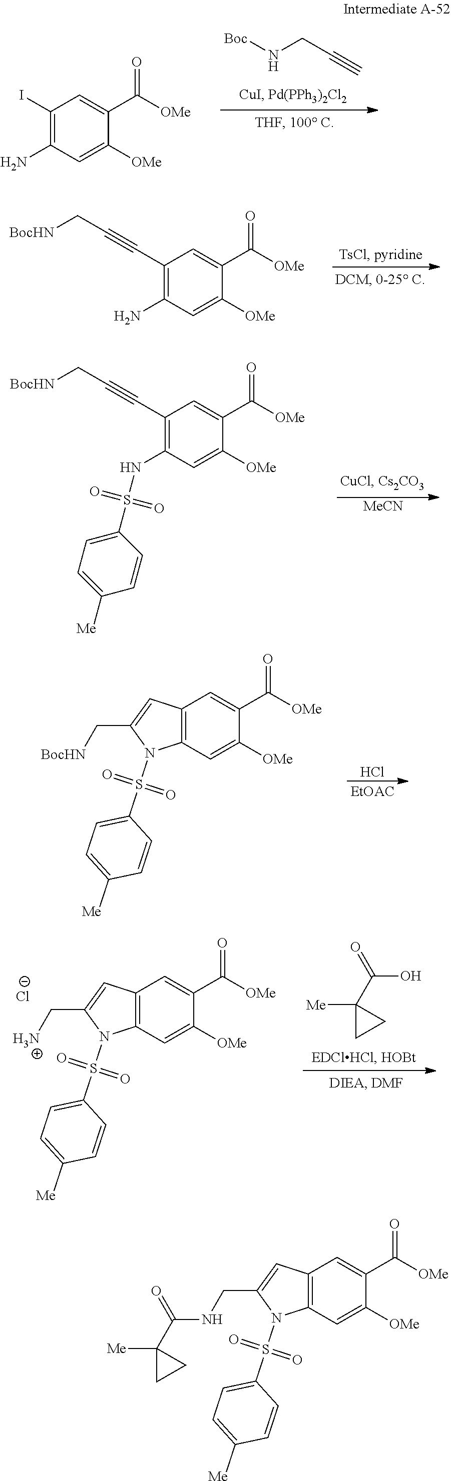

### Step 1: methyl 4-amino-5-(3-((tert-butoxycarbonyl)amino)prop-1-yn-1-yl)-2-methoxybenzoate

To a mixture of methyl 4-amino-5-iodo-2-methoxy-benzoate (110 g, 358 mmol, 1.00 eq) and tert-butyl N-prop-2-ynylcarbamate (55.6 g, 358 mmol, 1.00 eq) in THF (1 L) were added CuI (34.1 g, 179 mmol, 0.5 eq) and Pd(PPh₃)₂Cl₂ (25.1 g, 35.8 mmol, 0.1 eq). The reaction mixture was heated 100° C. for 16 h. The reaction mixture was cooled to room temperature and poured into water (1 L). The resulting mixture was then extracted with EtOAc (3×1 L). The combined organic layers were washed with brine (2×1 L), dried over anhydrous Na₂SO₄, filtered, and concentrated under reduced pressure. Purification by column chromatography gave methyl 4-amino-5-(3-((tert-butoxycarbonyl)amino)prop-1-yn-1-yl)-2-methoxybenzoate. LC-MS (ESI) m/z calcd for C₁₇H₂₂N₂O₅: 334.15; found: 279.2 [M−tBu+H]⁺.

### Step 2: methyl 5-(3-((tert-butoxycarbonyl)amino)prop-1-yn-1-yl)-2-methoxy-4-((4-methylphenyl)sulfonamido)benzoate

To a mixture of methyl 4-amino-5-(3-((tert-butoxycarbonyl)amino)prop-1-yn-1-yl)-2-methoxybenzoate (34.0 g, 101 mmol, 1.00 eq) and pyridine (16.4 mL, 203 mmol, 2.00 eq) in DCM (200 mL) was added 4-methylbenzenesulfonyl chloride (21.3 g, 111 mmol, 1.10 eq) at 0° C. The reaction mixture was warmed to 25° C. was stirred for 16 h before is was quenched by the addition of water (100 mL). The resulting mixture was extracted with EtOAc (3×150 mL). The combined organic layers were washed with brine (3×50 mL), dried over anhydrous Na₂SO₄, filtered, and concentrated under reduced pressure. The resulting crude residue was triturated with EtOAc (50 mL) at 25° C. for 20 min to give methyl 5-(3-((tert-butoxycarbonyl)amino)prop-1-yn-1-yl)-2-methoxy-4-((4-methylphenyl)sulfonamido)benzoate. LC-MS (ESI) m/z calcd for C₂₄H₂₈N₂O₇S: 488.16; found: 433.2 [M−tBu+H]⁺.

### Step 3: methyl 2-(((tert-butoxycarbonyl)amino)methyl)-6-methoxy-1-tosyl-1H-indole-5-carboxylate

To a solution of methyl 5-(3-((tert-butoxycarbonyl)amino)prop-1-yn-1-yl)-2-methoxy-4-((4-methylphenyl)sulfonamido)benzoate (10.6 g, 21.7 mmol, 1.00 eq) in MeCN (100 mL) were added Cs₂CO₃ (1.06 g, 3.25 mmol, 0.15 eq) and CuCl (322 mg, 3.25 mmol, 0.15 eq). The reaction mixture was stirred at 25° C. for 16 h. The reaction mixture was then filtered, and the filtrate was concentrated in vacuo. Purification by column chromatography gave methyl 2-(((tert-butoxycarbonyl)amino)methyl)-6-methoxy-1-tosyl-1H-indole-5-carboxylate. LC-MS (ESI) m/z calcd for C₂₄H₂₈N₂O₇S: 488.16; found: 489.2 [M+H]⁺.

### Step 4. (6-methoxy-5-(methoxycarbonyl)-1-tosyl-1H-indol-2-yl)methanaminium chloride

To a solution of methyl 2-(((tert-butoxycarbonyl)amino)methyl)-6-methoxy-1-tosyl-1H-indole-5-carboxylate (20.0 g, 40.9 mmol, 1.00 eq) in EtOAc (20 mL) was added HCl (4 M in EtOAc, 100 mL, 40.9 mmol, 1.00 eq). The reaction mixture was concentrated in vacuo to give (6-methoxy-5-(methoxycarbonyl)-1-tosyl-1H-indol-2-yl)methanaminium chloride. LC-MS (ESI) m/z calcd for C₁₉H₂₀N₂O₅S: 388.1; found: 389.2 [M+H]⁺.

### Step 5: methyl 2-(((tert-butoxycarbonyl)amino)methyl)-6-methoxy-1-tosyl-1H-indole-5-carboxylate

To a solution of 1-methylcyclopropanecarboxylic acid (3.06 g, 30.6 mmol, 1.00 eq) in DMF (130 mL) were added EDCI (7.62 g, 39.7 mmol, 1.3 eq) and HOBt (5.37 g, 39.7 mmol, 1.3 eq) and DIEA (11.9 g, 91.8 mmol, 3.00 eq). The reaction mixture was stirred for 30 min, then (6-methoxy-5-(methoxycarbonyl)-1-tosyl-1H-indol-2-yl)methanaminium chloride (13.0 g, 30.6 mmol, 1.00 eq) and DIEA (7.91 g, 61.1 mmol, 2.00 eq) were added and the reaction mixture was stirred at 25° C. for 16 h. The reaction mixture was poured into water (100 mL) and the mixture was stirred for 5 min and extracted with EtOAc (2×150 mL). The combined organic layers were washed with brine (3×150 mL), dried over anhydrous Na₂SO₄, filtered, and concentrated under reduced pressure. The resulting crude residue was triturated with EtOAc (30 mL) at 25° C. for 10 min to give methyl 2-(((tert-butoxycarbonyl)amino)methyl)-6-methoxy-1-tosyl-1H-indole-5-carboxylate. LC-MS (ESI) m/z calcd for C₂₄H₂₆N₂O₆S: 470.15; found: 471.2 [M+H]⁺.

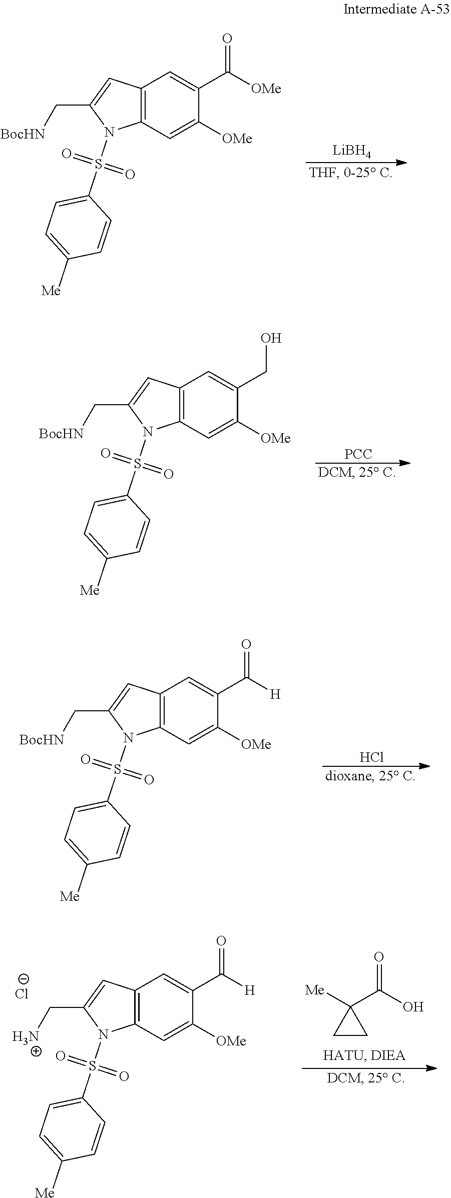

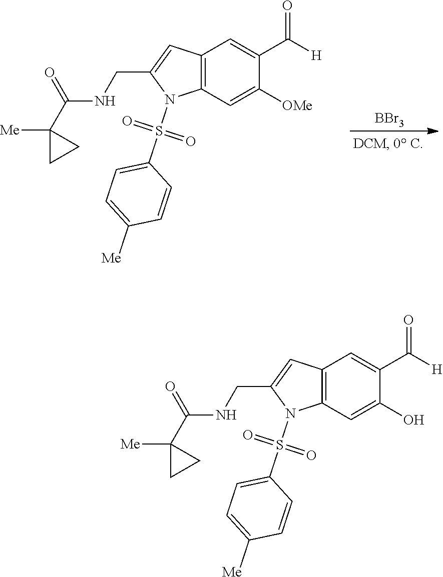 [SMILES: COc1cc2c(cc1C=O)cc(CNC(=O)C1(C)CC1)n2S(=O)(=O)c1ccc(C)cc1 ; "BBr3" ; Cc1ccc(S(=O)(=O)n2c(CNC(=O)C3(C)CC3)cc3cc(C=O)c(O)cc32)cc1]

### Step 1: tert-butyl ((5-(hydroxymethyl)-6-methoxy-1-tosyl-1H-indol-2-yl)methyl)carbamate

To a solution of 2-(((tert-butoxycarbonyl)amino)methyl)-6-methoxy-1-tosyl-1H-indole-5-carboxylate (3.60 g, 7.37 mmol, 1.00 eq) in THF (18 mL) was added LiBH₄ (2 M in THF, 22.1 mL, 6.00 eq) dropwise at 0° C. The reaction was allowed to warm to 25° C. After 16 h the reaction mixture was poured into saturated aqueous NH₄Cl (50 mL) slowly at 0° C. The solution was extracted with EtOAc (3×50 mL). The combined organic layers were washed with brine (50 mL), dried over anhydrous Na₂SO₄, filtered, and concentrated under reduced pressure. Purification by column chromatography gave tert-butyl ((5-(hydroxymethyl)-6-methoxy-1-tosyl-1H-indol-2-yl)methyl)carbamate (2.73 g). LC-MS (ESI) m/z calcd for C₂₃H₂₈N₂O₆S: 460.2; found 483.1 [M+Na]⁺.

### Step 2: tert-butyl ((5-formyl-6-methoxy-1-tosyl-1H-indol-2-yl)methyl)carbamate

To a solution of tert-butyl ((5-(hydroxymethyl)-6-methoxy-1-tosyl-1H-indol-2-yl)methyl)carbamate (3.18 g, 6.90 mmol, 1.00 eq) in DCM (32 mL) was added PCC (2.23 g, 10.4 mmol, 1.50 eq) at 25° C. After 1 h the reaction mixture was filtered through Celite. The filtrate was quenched by saturated aqueous Na₂SO₃ (30 mL) at 25° C. The solution was extracted with EtOAc (3×50 mL). The combined organic layers were washed with brine (50 mL), dried over anhydrous Na₂SO₄, filtered, and concentrated under reduced pressure. Purification by column chromatography gave tert-butyl ((5-formyl-6-methoxy-1-tosyl-1H-indol-2-yl)methyl)carbamate (2.69 g). LC-MS (ESI) m/z calcd for C₂₃H₂₆N₂O₆S: 458.2; found 459.1 [M+H]⁺.

### Step 3: 2-(aminomethyl)-6-methoxy-1-tosyl-1H-indole-5-carbaldehyde hydrochloride

To a solution of tert-butyl ((5-formyl-6-methoxy-1-tosyl-1H-indol-2-yl)methyl)carbamate (700 mg, 1.53 mmol, 1.00 eq) in dioxane (3 mL) was added HCl/dioxane (4 M, 10 mL, 26.2 eq) dropwise at 0° C. The reaction was warmed to 25° C. and stirred for 1 h. The reaction mixture was concentrated under reduced pressure to give 2-(aminomethyl)-6-methoxy-1-tosyl-1H-indole-5-carbaldehyde hydrochloride (670 mg, HCl salt), which was used directly without further purification. LC-MS (ESI) m/z calcd for C₁₈H₁₈N₂O₄S: 358.1; found 342.1 [M−NH₂]⁺.

### Step 4: N-((5-formyl-6-methoxy-1-tosyl-1H-indol-2-yl)methyl)-1-methylcyclopropane-1-carboxamide

To a solution of 2-(aminomethyl)-6-methoxy-1-tosyl-1H-indole-5-carbaldehyde hydrochloride (620 mg, 1.57 mmol, 1.00 eq, HCl) in DCM (6 mL) was added 1-methylcyclopropanecarboxylic acid (189 mg, 1.88 mmol, 1.20 eq), HATU (776 mg, 2.04 mmol, 1.30 eq), and DIEA (1.22 g, 9.42 mmol, 6.00 eq) in one portion. The reaction mixture was stirred at 25° C. After 1 h, the reaction was quenched by addition of H₂O (15 mL). The solution was extracted with EtOAc (3×15 mL). The combined organic layers were washed with brine (15 mL), dried over anhydrous Na₂SO₄, filtered, and concentrated under reduced pressure. Purification by column chromatography gave N-((5-formyl-6-methoxy-1-tosyl-1H-indol-2-yl)methyl)-1-methylcyclopropane-1-carboxamide (290 mg). LC-MS (ESI) m/z calcd for C₂₃H₂₄N₂O₅S: 440.1; found 441.1 [M+H]⁺.

### Step 5: N-((5-formyl-6-hydroxy-1-tosyl-1H-indol-2-yl)methyl)-1-methylcyclopropane-1-carboxamide

To a solution of N-((5-formyl-6-methoxy-1-tosyl-1H-indol-2-yl)methyl)-1-methylcyclopropane-1-carboxamide (300 mg, 681 μmol, 1.00 eq) in DCM (5 mL) was added BBr₃ (171 mg, 681 μmol, 1.00 eq) dropwise at 0° C. The reaction mixture was stirred at 0° C. After 2 h the reaction mixture was poured into H₂O (10 mL) slowly at 25° C. under N₂ atmosphere. The pH was adjusted to 7-8 with saturated aqueous Na₂CO₃. The mixture was extracted with EtOAc (3×20 mL). The combined organic layers were washed with brine (20 mL), dried over anhydrous Na₂SO₄, filtered, and concentrated under reduced pressure. Purification by column chromatography gave N-((5-formyl-6-hydroxy-1-tosyl-1H-indol-2-yl)methyl)-1-methylcyclopropane-1-carboxamide (200 mg). LC-MS (ESI) m/z calcd for C₂₂H₂₂N₂O₅S: 426.1; found 427.1 [M+H]⁺.

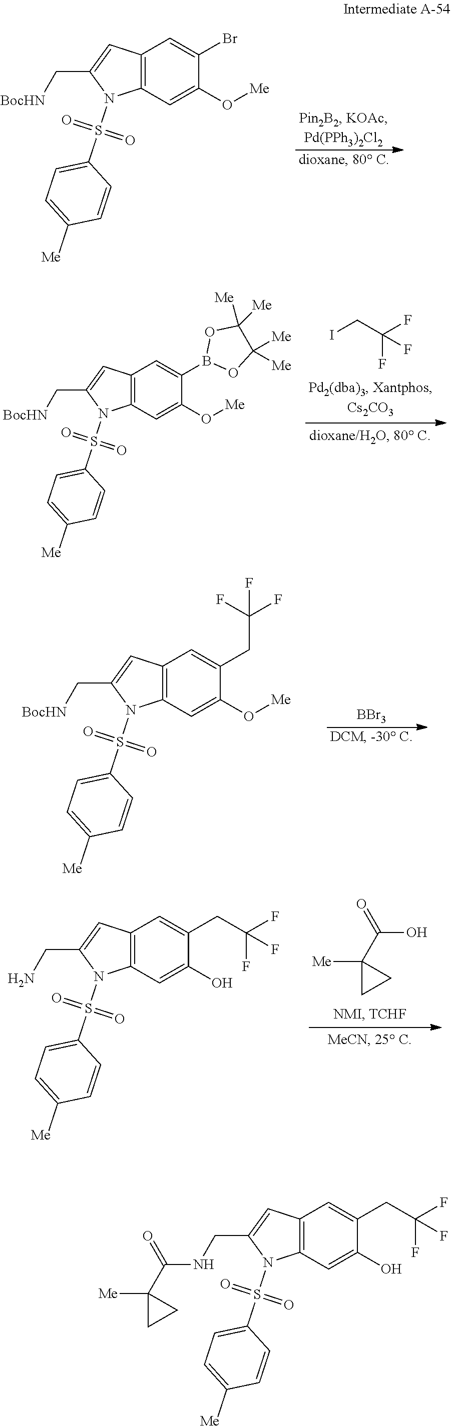

### Step 1: tert-butyl ((6-methoxy-5-(4,4,5,5-tetramethyl-1,3,2-dioxaborolan-2-yl)-1-tosyl-1H-indol-2-yl)methyl)carbamate

To a mixture of tert-butyl ((5-bromo-6-methoxy-1-tosyl-1H-indol-2-yl)methyl)carbamate (17.0 g, 33.37 mmol, 1.00 eq) and Pin₂B₂ (25.4 g, 100 mmol, 3.00 eq) in dioxane (200 mL) was added KOAc (8.19 g, 83.4 mmol, 2.50 eq) and Pd(PPh₃)₂Cl₂ (1.17 g, 1.67 mmol, 0.05 eq). The reaction mixture was heated to 80° C. and stirred for 10 h under an atmosphere of N₂. The reaction mixture was allowed to cool to room temperature, diluted with water (450 mL), and extracted with EtOAc (3×150 mL). The combined organic phase was washed with brine (150 mL), dried over Na₂SO₄, filtered, and concentrated under reduced pressure. Purification by column chromatography gave tert-butyl N-[[6-methoxy-1-(p-tolylsulfonyl)-5-(4,4,5,5-tetramethyl-1,3,2-dioxaborolan-2-yl)indol-2-yl]methyl]carbamate. LC-MS (ESI) m/z calcd for C₂₈H₃₇BN₂O₇S: 556.24; found: 501.1 [M−tBu+2H]⁺.

### Step 2: tert-butyl ((6-methoxy-1-tosyl-5-(2,2,2-trifluoroethyl)-1H-indol-2-yl)methyl)carbamate

To six parallel mixtures of tert-butyl N-[[6-methoxy-1-(p-tolylsulfonyl)-5-(4,4,5,5-tetramethyl-1,3,2-dioxaborolan-2-yl)indol-2-yl]methyl]carbamate (0.60 g, 1.08 mmol, 1.00 eq) and 1,1,1-trifluoro-2-iodo-ethane (4.53 g, 21.6 mmol, 20.0 eq) in 1,4-dioxane (5 mL) and H₂O (1 mL) were added Cs₂CO₃ (703 mg, 2.16 mmol, 2.00 eq), Pd₂(dba)₃ (99.0 mg, 108 μmol, 0.10 eq) and Xantphos (125 mg, 216 μmol, 0.20 eq). The reaction mixtures were degassed and purged with N₂ three times, then warmed to 80° C. with microwave irradiation and stirred for 16 h. The six reaction mixtures were then combined. The combined mixture was diluted with water (50 mL), extracted with EtOAc (3×50 mL). The combined organic phase was washed with brine (50 mL), dried over Na₂SO₄, filtered, and concentrated under reduced pressure. Purification by column chromatography gave tert-butyl ((6-methoxy-1-tosyl-5-(2,2,2-trifluoroethyl)-1H-indol-2-yl)methyl)carbamate.

### Step 3: 2-(aminomethyl)-1-tosyl-5-(2,2,2-trifluoroethyl)-1H-indol-6-ol

To a solution of tert-butyl ((6-methoxy-1-tosyl-5-(2,2,2-trifluoroethyl)-1H-indol-2-yl)methyl)carbamate (1.50 g, 2.93 mmol, 1.00 eq) in DCM (30 mL) cooled to −30° C. was added BBr₃ (2.20 g, 8.78 mmol, 3.00 eq) dropwise. The reaction mixture was stirred at −30° C. for 2 h. The reaction mixture was warmed to 0° C. and MeOH (50 mL) was added dropwise under an atmosphere of N₂. The resulting mixture was stirred for 30 min and then concentrated under reduced pressure. The residue was triturated with MTBE/EtOAc=2/1 (2×30 mL) and then filtered. Collection of the filter cake gave 2-(aminomethyl)-1-tosyl-5-(2,2,2-trifluoroethyl)-1H-indol-6-ol. LC-MS (ESI) m/z calcd for C₁₈H₁₇F₃N₂O₃S: 398.09; found: 382.0 [M−NH₂+H]⁺.

### Step 4: N-((6-hydroxy-1-tosyl-5-(2,2,2-trifluoroethyl)-1H-indol-2-yl)methyl)-1-methylcyclopropane-1-carboxamide

To a mixture of 2-(aminomethyl)-1-tosyl-5-(2,2,2-trifluoroethyl)-1H-indol-6-ol (800 mg, 2.01 mmol, 1.00 eq) and 1-methylcyclopropanecarboxylic acid (241 mg, 2.41 mmol, 1.20 eq) in MeCN (15 mL) cooled to 0° C. was added 1-methyl-1H-imidazole (NMI, 824 mg, 10.0 mmol, 5.00 eq) and N-(chloro(dimethylamino)methylene)-N-methylmethanaminium hexafluorophosphate(V) (TCFH, 676 mg, 2.41 mmol, 1.20 eq) in portions. The reaction mixture was allowed to warm to 25° C. and stirred for 1 h. The reaction mixture was diluted with water (60 mL), extracted with EtOAc (3×20 mL). The combined organic phase was washed with brine (20 mL), dried over Na₂SO₄, filtered, and concentrated under reduced pressure. Purification by column chromatography gave N-((6-hydroxy-1-tosyl-5-(2,2,2-trifluoroethyl)-1H-indol-2-yl)methyl)-1-methylcyclopropane-1-carboxamide. LC-MS (ESI) m/z calcd for C₂₃H₂₃F₃N₂O₄S: 480.13; found: 481.2 [M+H]⁺.

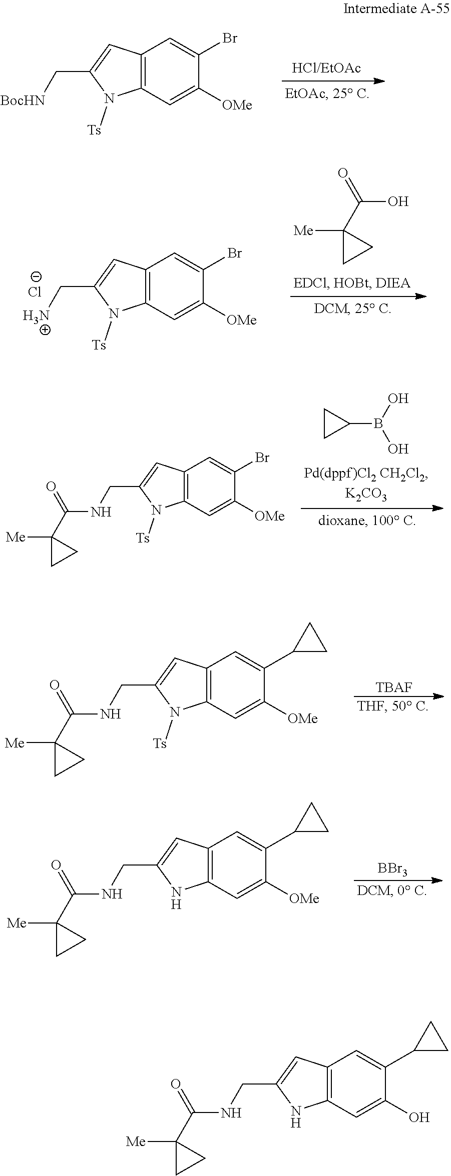

### Step 1: (5-bromo-6-methoxy-1-tosyl-1H-indol-2-yl)methanamine hydrochloride

To a solution of tert-butyl ((5-bromo-6-methoxy-1-tosyl-1H-indol-2-yl)methyl)carbamate (5.00 g, 9.82 mmol, 1.00 eq) in EtOAc (10 mL) was added HCl/EtOAc (4 M in EtOAc, 50 mL, 20.4 eq). The resulting reaction mixture was stirred at 25° C. for 1 h. The reaction mixture was concentrated under reduced pressure to give (5-bromo-6-methoxy-1-tosyl-1H-indol-2-yl)methanamine hydrochloride, which was used for next step directly without of further purification. LC-MS (ESI) m/z calcd for C₁₇H₁₇BrN₂O₃S: 408.01; found: 392.0 [M−NH₂]⁺.

### Step 2: N-((5-bromo-6-methoxy-1-tosyl-1H-indol-2-yl)methyl)-1-methylcyclopropane-1-carboxamide

To a solution of 1-methylcyclopropanecarboxylic acid (1.16 g, 11.6 mmol, 1.20 eq) in DCM (50 mL) was added EDCI (2.22 g, 11.6 mmol, 1.20 eq) and HOBt (1.56 g, 11.6 mmol, 1.20 eq). The reaction mixture was stirred at 25° C. for 0.5 h. Then, DIEA (4.99 g, 38.6 mmol, 4.00 eq) and (5-bromo-6-methoxy-1-tosyl-1H-indol-2-yl)methanamine hydrochloride (4.30 g, 9.65 mmol, 1.00 eq, HCl salt) were added. The reaction mixture was stirred at 25° C. for 1 h. The reaction was quenched with H₂O (20 mL), and the aqueous phase was extracted with ethyl acetate (3×30 mL). The combined organic layers were washed with brine (20 mL), dried over anhydrous Na₂SO₄, filtered, and concentrated under reduced pressure. The resulting residue was triturated with ethyl acetate (10 ml) at 25° C. for 10 mins and filtered. The filter cake was collected to give N-((5-bromo-6-methoxy-1-tosyl-1H-indol-2-yl)methyl)-1-methylcyclopropane-1-carboxamide. LC-MS (ESI) m/z calcd C₂₂H₂₃BrN₂O₄S: 490.06; found: 491.1 [M+H]⁺.

### Step 3: N-((5-cyclopropyl-6-methoxy-1-tosyl-1H-indol-2-yl)methyl)-1-methylcyclopropane-1-carboxamide

To a mixture of cyclopropylboronic acid (1.31 g, 15.3 mmol, 5.00 eq) and N-((5-bromo-6-methoxy-1-tosyl-1H-indol-2-yl)methyl)-1-methylcyclopropane-1-carboxamide (1.50 g, 3.05 mmol, 1.00 eq) in dioxane (15 mL) as added cyclopentyl(diphenyl)phosphane dichloromethane dichloropalladium iron (499 mg, 611 μmol, 0.20 eq) and K₂CO₃ (1.27 g, 9.16 mmol, 3.00 eq) under N₂ atmosphere. The resulting reaction mixture was heated to 100° C. and stirred for 12 h. After cooling to room temperature, the reaction mixture was poured into H₂O (10 mL), the aqueous phase was extracted with ethyl acetate (3×20 mL). The combined organic phase was washed with brine (2×10 mL), dried over anhydrous Na₂SO₄, filtered, and concentrated under reduced pressure. Purification by column chromatography gave N-((5-cyclopropyl-6-methoxy-1-tosyl-1H-indol-2-yl)methyl)-1-methylcyclopropane-1-carboxamide. LC-MS (ESI) m/z calcd for C₂₅H₂₈N₂O₄S: 452.18; found: 453.2 [M+H]⁺.

### Step 4: N-((5-cyclopropyl-6-methoxy-1H-indol-2-yl)methyl)-1-methylcyclopropane-1-carboxamide

To a solution of N-((5-cyclopropyl-6-methoxy-1-tosyl-1H-indol-2-yl)methyl)-1-methylcyclopropane-1-carboxamide (1.20 g, 2.65 mmol, 1.00 eq) in THF (2 mL) was added TBAF (1 M in THF, 53 mL, 20.0 eq) dropwise at 25° C., then the reaction mixture was heated to 50° C. and stirred for 12 h. After cooling to room temperature, the reaction mixture was poured into ice-water (8 mL), the aqueous phase was extracted with ethyl acetate (3×15 mL). The combined organic phase was washed with saturated aqueous NH₄Cl solution (3×15 mL), dried over anhydrous Na₂SO₄, filtered, and concentrated under reduced pressure. Purification by column chromatography gave N-((5-cyclopropyl-6-methoxy-1H-indol-2-yl)methyl)-1-methylcyclopropane-1-carboxamide. LC-MS (ESI) m/z calcd for C₁₈H₂₂N₂O₂: 298.17; found: 299.2 [M+H]⁺.

### Step 5: N-((5-cyclopropyl-6-hydroxy-1H-indol-2-yl)methyl)-1-methylcyclopropane-1-carboxamide

To a solution of N-((5-cyclopropyl-6-methoxy-1H-indol-2-yl)methyl)-1-methylcyclopropane-1-carboxamide (280 mg, 938 μmol, 1.00 eq) in DCM (3 mL) was added BBr₃ (940 mg, 3.75 mmol, 362 μL, 4.00 eq) dropwise at 0° C. under N₂. The reaction mixture was stirred at 0° C. for 0.5 h before being poured into ice-water (5 mL) and stirred for 2 mins. The aqueous phase was basified with saturated aqueous NaHCO₃ solution to pH 8 and extracted with ethyl acetate (10 mL×3). The combined organic phase was washed with brine (10 mL), dried over anhydrous Na₂SO₄, filtered, and concentrated under reduced pressure. Purification by column chromatography gave N-((5-cyclopropyl-6-hydroxy-1H-indol-2-yl)methyl)-1-methylcyclopropane-1-carboxamide. LC-MS (ESI) m/z calcd for C₁₇H₂₀N₂O₂: 284.15; found: 285.2 [M+H]⁺.

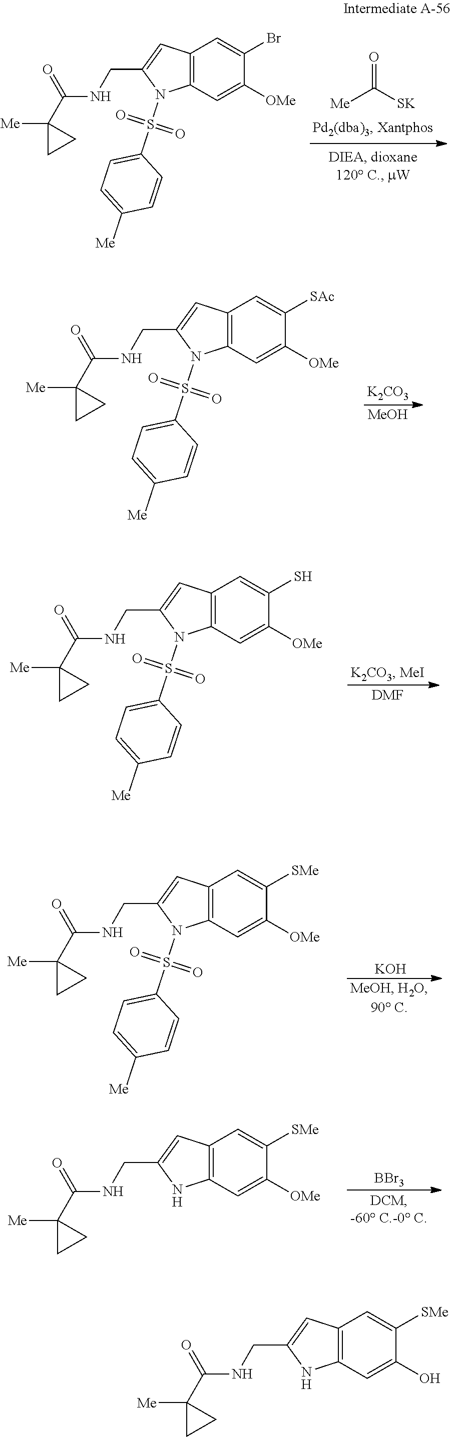

### Step 1: S-(6-methoxy-2-((1-methylcyclopropane-1-carboxamido)methyl)-1-tosyl-1H-indol-5-yl) ethanethioate

To a mixture of N-((5-bromo-6-methoxy-1-tosyl-1H-indol-2-yl)methyl)-1-methylcyclopropane-1-carboxamide (1.10 g, 2.24 mmol, 1.00 eq) and potassium ethanethioate (1.36 g, 11.9 mmol, 5.3 eq) in dioxane (11 mL) was added DIEA (578 mg, 4.48 mmol, 2.00 eq). The mixture was degassed and purged with N₂ three times. Pd₂(dba)₃ (204 mg, 223 μmol, 0.1 eq) and Xantphos (259 mg, 447 μmol, 0.2 eq) were added and the reaction was heated to 120° C. under microwave and stirred for 3 h. The reaction mixture was cooled to room temperature and quenched with water (50 mL). The resulting mixture was extracted with EtOAc (2×30 mL). The combined organic layers were washed with brine (50 mL), dried over anhydrous Na₂SO₄, filtered, and concentrated under reduced pressure. Purification by column chromatography to give S-(6-methoxy-2-((1-methylcyclopropane-1-carboxamido)methyl)-1-tosyl-1H-indol-5-yl) ethanethioate. LC-MS (ESI) m/z calcd for C₂₄H₂₆N₂O₅S₂: 486.13; found: 487.3 [M+H]⁺.

### Step 2: N-((5-mercapto-6-methoxy-1-tosyl-1H-indol-2-yl)methyl)-1-methylcyclopropane-1-carboxamide

To a solution of S-(6-methoxy-2-((1-methylcyclopropane-1-carboxamido)methyl)-1-tosyl-1H-indol-5-yl) ethanethioate (2.26 g, 4.63 mmol, 1.00 eq) in MeOH (20 mL) was added K₂CO₃ (1.93 g, 13.9 mmol, 3.00 eq) and the reaction was stirred at 25° C. for 1 h. The reaction mixture was quenched with water (30 mL). The resulting mixture was extracted with EtOAc (2×30 mL). The combined organic layers were washed with brine (30 mL), dried over anhydrous Na₂SO₄, filtered, and concentrated under reduced pressure to give N-((5-mercapto-6-methoxy-1-tosyl-1H-indol-2-yl)methyl)-1-methylcyclopropane-1-carboxamide. LC-MS (ESI) m/z calcd for C₂₂H₂₄N₂O₄S₂: 444.12; found: 445.1 [M+H]⁺.

### Step 3: N-((6-methoxy-5-(methylthio)-1-tosyl-1H-indol-2-yl)methyl)-1-methylcyclopropane-1-carboxamide

To a solution of N-((5-mercapto-6-methoxy-1-tosyl-1H-indol-2-yl)methyl)-1-methylcyclopropane-1-carboxamide (1.80 g, 4.05 mmol, 1.00 eq) in DMF (13 mL) were added K₂CO₃ (1.12 g, 8.10 mmol, 2.00 eq) and Mel (689 mg, 4.86 mmol, 1.2 eq), and the reaction mixture was stirred at 25° C. for 1 h. The reaction mixture was quenched with water (20 mL). The resulting mixture was extracted with EtOAc (2×20 mL). The combined organic layers were washed with brine (20 mL), dried over anhydrous Na₂SO₄, filtered, and concentrated under reduced pressure. Purification by column chromatography to give N-((6-methoxy-5-(methylthio)-1-tosyl-1H-indol-2-yl)methyl)-1-methylcyclopropane-1-carboxamide. LC-MS (ESI) m/z calcd for C₂₃H₂₆N₂O₄S₂: 458.13; found: 459.4 [M+H]⁺.

### Step 4: N-((6-methoxy-5-(methylthio)-1H-indol-2-yl)methyl)-1-methylcyclopropane-1-carboxamide

To a solution of N-((6-methoxy-5-(methylthio)-1-tosyl-1H-indol-2-yl)methyl)-1-methylcyclopropane-1-carboxamide (1.20 g, 2.62 mmol, 1.00 eq) in MeOH (12 mL) and H₂O (4 mL) was added KOH (734 mg, 13.0 mmol, 2.00 eq) and the reaction mixture was heated to 90° C. for 16 h. The reaction mixture was cooled to 25° C. and quenched with water (20 mL). The resulting mixture was extracted with EtOAc (2×20 mL). The combined organic layers were washed with brine (20 mL), dried over anhydrous Na₂SO₄, filtered, and concentrated under reduced pressure to give N-((6-methoxy-5-(methylthio)-1H-indol-2-yl)methyl)-1-methylcyclopropane-1-carboxamide. LC-MS (ESI) m/z calcd for C₁₆H₂₀N₂O₂S₂: 304.12; found: 305.0 [M+H]⁺.

### Step 5: N-((6-hydroxy-5-(methylthio)-1H-indol-2-yl)methyl)-1-methylcyclopropane-1-carboxamide

To a solution N-((6-methoxy-5-(methylthio)-1H-indol-2-yl)methyl)-1-methylcyclopropane-1-carboxamide (0.75 g, 2.46 mmol, 1.00 eq) in DCM (8 mL) was added BBr₃ (2.47 g, 9.86 mmol, 4.00 eq) dropwise at −60° C. The reaction was warmed to 0° C. and stirred for 1 h. The reaction mixture was quenched with water (20 mL) and was adjusted to pH 8 with the addition of sat. aq. NaHCO₃. The mixture was extracted with EtOAc (2×30 mL). The combined organic layers were washed with brine (30 mL), dried over anhydrous Na₂SO₄, filtered, and concentrated under reduced pressure. Purification by column chromatography gave N-((6-hydroxy-5-(methylthio)-1H-indol-2-yl)methyl)-1-methylcyclopropane-1-carboxamide. LC-MS (ESI) m/z calcd for C₁₅H₁₈N₂O₂S: 290.11; found: 291.3 [M+H]⁺.

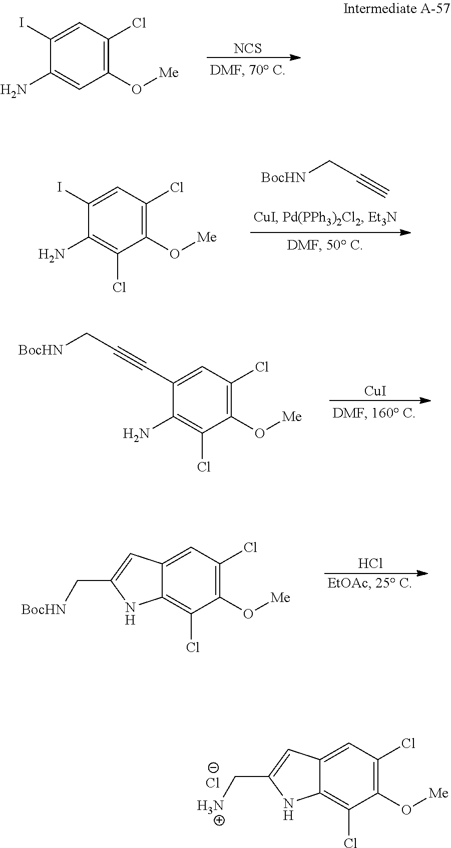

### Step 1: 2,4-dichloro-6-iodo-3-methoxyaniline

To a solution of 4-chloro-2-iodo-5-methoxyaniline (5.00 g, 17.6 mmol, 1.00 eq) in DMF (10 mL) was added NCS (2.36 g, 17.6 mmol, 1.00 eq) in portions. The reaction mixture was warmed to 70° C. and stirred for 2 h. The reaction mixture was allowed to cool to room temperature and poured into H₂O (30 mL) and stirred for 5 min. The aqueous phase was extracted with ethyl acetate (2×30 mL). The combined organic phase was washed with brine (2×30 mL), dried over anhydrous Na₂SO₄, filtered, and concentrated under reduced pressure. Purification by column chromatography gave 2,4-dichloro-6-iodo-3-methoxyaniline. LC-MS (ESI) m/z calcd for C₇H₆Cl₂INO: 316.89; found: 317.9 [M+H]⁺.

### Step 2: tert-butyl (3-(2-amino-3,5-dichloro-4-methoxyphenyl)prop-2-yn-1-yl)carbamate

To a solution of 2,4-dichloro-6-iodo-3-methoxyaniline (3.50 g, 11.0 mmol, 1.00 eq) and tert-butyl prop-2-yn-1-ylcarbamate (4.27 g, 27.5 mmol, 2.50 eq) in DMF (20 mL) was added CuI (419 mg, 2.20 mmol, 0.2 eq), Pd(PPh₃)₂Cl₂ (772 mg, 1.10 mmol, 0.1 eq) and TEA (55.0 mmol, 7.66 mL, 5.0 eq). The reaction mixture was degassed and charged with nitrogen three times. The reaction mixture was warmed to 50° C. and stirred for 16 h. The reaction mixture was allowed to cool to room temperature and was poured into H₂O (100 mL) and stirred for 5 min. The aqueous phase was extracted with ethyl acetate (2×30 mL) and the combined organic phase was washed with brine (2×30 mL), dried over anhydrous Na₂SO₄, filtered, and concentrated under reduced pressure. Purification by column chromatography gave (tert-butyl (3-(2-amino-3,5-dichloro-4-methoxyphenyl)prop-2-yn-1-yl)carbamate. LC-MS (ESI) m/z calcd for C₁₅H₁₈Cl₂N₂O₃: 344.07; found: 289.1 [M−t-Bu+2H]⁺.

### Step 3: tert-butyl ((5,7-dichloro-6-methoxy-1H-indol-2-yl)methyl)carbamate

To a solution of tert-butyl (3-(2-amino-3,5-dichloro-4-methoxyphenyl)prop-2-yn-1-yl)carbamate (1.50 g, 4.35 mmol, 1.0 eq) in DMF (20 mL) was added CuI (82.7 mg, 434 μmol, 0.1 eq). The reaction mixture was heated to 160° C. and stirred for 1 h. The reaction mixture was allowed to cool to room temperature, poured into H₂O (100 mL), and stirred for 5 min. The aqueous phase was extracted with ethyl acetate (2×30 mL). The combined organic phase was washed with brine (2×10 mL), dried over anhydrous Na₂SO₄, filtered, and concentrated under reduced pressure. Purification by column chromatography gave tert-butyl ((5,7-dichloro-6-methoxy-1H-indol-2-yl)methyl)carbamate. LC-MS (ESI) m/z calcd for C₁₅H₁₈Cl₂N₂O₃: 344.07; found: 289.0 [M−t-Bu+2H]⁺.

### Step 4: (5,7-dichloro-6-methoxy-1H-indol-2-yl)methanamine hydrochloride

To a solution of tert-butyl ((5,7-dichloro-6-methoxy-1H-indol-2-yl)methyl)carbamate (1.00 g, 2.90 mmol, 1.00 eq) in EtOAc (10 mL) was added HCl (4 M solution in EtOAc, 10 mL, 13.8 eq) in portions. The reaction mixture was stirred at 25° C. for 1 h. Concentration of the reaction mixture under reduced pressure gave (5,7-dichloro-6-methoxy-1H-indol-2-yl)methanamine hydrochloride, which was used without further purification. LC-MS (ESI) m/z calcd for C₁₀H₁₀Cl₂N₂O: 244.02; found: 228.0 [M−NH₂]⁺.

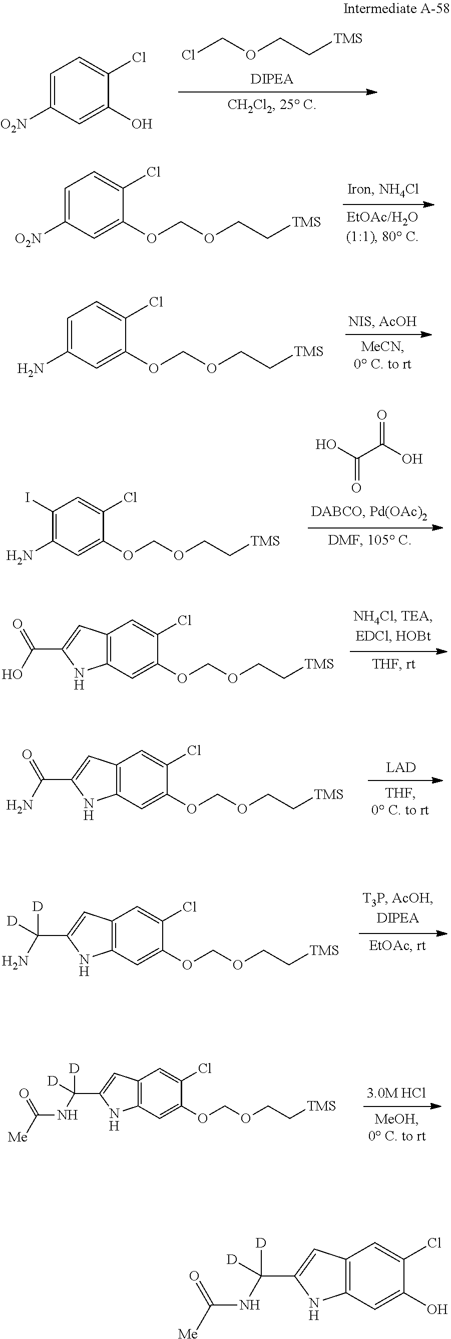

### Step 1: (2-((2-chloro-5-nitrophenoxy)methoxy)ethyl)trimethylsilane

To a solution of 2-chloro-5-nitrophenol (5.0 g, 28.8 mmol, 1.0 eq) in methylene chloride (28.8 mL) was added DIPEA (10.0 mL, 57.6 mmol, 2.0 eq) and (2-(chloromethoxy)ethyl)trimethylsilane sequentially at room temperature. After 16 h, sat. aq. NH₄Cl (28.8 mL) was added and the organic layer was separated. The aqueous layer was further extracted with methylene chloride (3×10 mL). The combined organic layers were dried over anhydrous Na₂SO₄, filtered, and concentrated under reduced pressure to obtain (2-((2-chloro-5-nitrophenoxy)methoxy)ethyl)trimethylsilane, which was used without further purification. LC-MS (ESI) m/z calcd for C₁₂H₁₈ClNO₄Si: 303.1; found: 304.1 [M+H]⁺.

### Step 2: 4-chloro-3-((2-(trimethylsilyl)ethoxy)methoxy)aniline

To a solution of crude (2-((2-chloro-5-nitrophenoxy)methoxy)ethyl)trimethylsilane from the previous step in EtOAc/water solution (1:1, 40 mL) was added iron (8.05 g, 144.1 mmol, 5 eq) and ammonium chloride (7.7 g, 144.1 mmol, 5.0 eq). A reflux condenser was fitted, and the reaction mixture was heated to reflux for 2 hours. After cooling to room temperature, the reaction mixture was filtered the remove all solids. To the resulting biphasic mixture was added sat. aq. sodium bicarbonate solution with vigorous stirring until gas evolution ceased. The aqueous phase was extracted with EtOAc (3×15 mL). The combined organic layers were dried over anhydrous Na₂SO₄, filtered, and concentrated under reduced pressure to give 4-chloro-3-((2-(trimethylsilyl)ethoxy)methoxy)aniline, which was used without further purification. LC-MS (ESI) m/z calcd for C₁₂H₂₀ClNO₂Si: 273.1; found: 274.1 [M+H]⁺.

### Step 3: 4-chloro-2-iodo-5-((2-(trimethylsilyl)ethoxy)methoxy)aniline

To a solution of 4-chloro-3-((2-(trimethylsilyl)ethoxy)methoxy)aniline from the previous step in MeCN (50 mL) was added acetic acid (2 drops) and NIS (7.1 g, 31.6 mmol, 1.2 eq) at 0° C. The reaction mixture was allowed to slowly warm to room temperature. After 1 h, sat. aq. NaHCO₃ (50 mL) was added. The aqueous phase was extracted with EtOAc (3×20 mL). The combined organic layers were dried over anhydrous Na₂SO₄, filtered, and concentrated under reduced pressure. Purification by column chromatography gave 4-chloro-2-iodo-5-((2-(trimethylsilyl)ethoxy)methoxy)aniline. LC-MS (ESI) m/z calcd for C₁₂H₁₉ClINO₂Si: 399.0; found: 400.1 [M+H]⁺.

### Step 4: 5-chloro-6-((2-(trimethylsilyl)ethoxy)methoxy)-1H-indole-2-carboxylic acid

To a solution of 4-chloro-2-iodo-5-((2-(trimethylsilyl)ethoxy)methoxy)aniline (2.0 g, 5.0 mmol, 1.0 eq) in DMF (15 mL) was added pyruvic acid (1.3 g, 15.0 mmol, 3 eq), DABCO (1.7 g, 15.0 mmol, 3 eq), and palladium acetate (56 mg, 0.25 mmol, 5 mol %, 0.05 eq). The sealed vessel was evacuated and purge with N₂ four times then heated to 105° C. for 16 hours. After cooling to room temperature, the reaction mixture was poured into ice water (120 mL) and extracted with EtOAc (4×50 mL). The combined organic layers were dried over anhydrous Na₂SO₄, filtered, and concentrated under reduced pressure to give 5-chloro-6-((2-(trimethylsilyl)ethoxy)methoxy)-1H-indole-2-carboxylic acid, which was used without further purification. LC-MS (ESI) m/z calcd for C₁₅H₂₀ClNO₄Si: 341.1; found: 340.2 [M−H]⁻.

### Step 5: 5-chloro-6-((2-(trimethylsilyl)ethoxy)methoxy)-1H-indole-2-carboxamide

To a solution of 5-chloro-6-((2-(trimethylsilyl)ethoxy)methoxy)-1H-indole-2-carboxylic acid in THF (12.6 mL) was added EDCI (1.2 g, 6.0 mmol, 1.2 eq) and HOBt (1.2 g, 6.0 mmol, 1.2 eq) sequentially. The resulting mixture was stirred for 30 minutes before ammonium chloride (404.4 mg, 7.56 mmol, 1.5 eq) and TEA (1.05 mL 7.56 mmol, 1.5 eq) were added sequentially. After 16 h the reaction was quenched with sat. aq. NH₄Cl (20 mL). The aqueous phase was extracted with EtOAc (3×10 mL). The combined organic layers were dried over anhydrous Na₂SO₄, filtered, and concentrated under reduced pressure to give 5-chloro-6-((2-(trimethylsilyl)ethoxy)methoxy)-1H-indole-2-carboxamide, which was used without further purification. LC-MS (ESI) m/z calcd for C₁₅H₂₁ClN₂O₃Si: 340.1; found: 341.2 [M+H]⁺.

### Step 6: (5-chloro-6-((2-(trimethylsilyl)ethoxy)methoxy)-1H-indol-2-yl)methan-d₂-amine

To a solution of 5-chloro-6-((2-(trimethylsilyl)ethoxy)methoxy)-1H-indole-2-carboxamide (606 mg, 1.8 mmol, 1.0 eq) in THF (6 mL) was added lithium aluminum deuteride (223.9 mg, 5.3 mmol, 3.0 eq) in three equal portions at 0° C. The reaction was allowed to warm slowly to room temperature. After 16 h diethyl ether (6 mL) was added, and the mixture cooled to 0° C. To the cooled mixture, water (0.25 mL) was added dropwise. The reaction mixture was further diluted with 15% aq. sodium hydroxide (0.25 mL) and water (0.75 mL). The mixture was warmed to room temperature and stirred for 15 min. Magnesium sulfate was added and stirring was continued for 15 min. Solids were then removed by filtration, and the aqueous phase was extracted with EtOAc (2×5 mL). The combined organic layers were dried over anhydrous Na₂SO₄, filtered, and concentrated under reduced pressure to give (5-chloro-6-((2-(trimethylsilyl)ethoxy)methoxy)-1H-indol-2-yl)methan-d₂-amine, which was used without further purification. LC-MS (ESI) m/z calcd for C₁₅H₂₁D₂ClN₂O₂Si: 328.1; found: 329.2 [M+H]⁺.

### Step 7: N-((5-chloro-6-((2-(trimethylsilyl)ethoxy)methoxy)-1H-indol-2-yl)methyl-d₂)acetamide

To a solution of (5-chloro-6-((2-(trimethylsilyl)ethoxy)methoxy)-1H-indol-2-yl)methan-d₂-amine (675 mg, 2.1 mmol, 1.0 eq) in EtOAc (3 mL) was added DIPEA (3.6 mL, 20.5 mmol, 10.0 eq), acetic acid (0.47 mL, 8.2 mmol, 4.0 eq), and 50% T₃P in EtOAc (6.1 mL, 10.3 mmol, 5.0 eq) sequentially. After 16 h, sat. aqueous NaHCO₃ (6 mL) was added. The aqueous phase was extracted using EtOAc (3×2 mL). The combined organic layers were dried over anhydrous Na₂SO₄, filtered, and concentrated under reduced pressure. Purification by column chromatography gave N-((5-chloro-6-((2-(trimethylsilyl)ethoxy)methoxy)-1H-indol-2-yl)methyl-d₂)acetamide. LC-MS (ESI) m/z calcd for C₁₇H₂₃D₂ClN₂O₃Si: 370.1; found: 371.2 [M+H]⁺.

### Step 8: N-((5-chloro-6-hydroxy-1H-indol-2-yl)methyl-d₂)acetamide

To a solution of N-((5-chloro-6-((2-(trimethylsilyl)ethoxy)methoxy)-1H-indol-2-yl)methyl-d₂)acetamide (260 mg, 0.7 mmol, 1.0 eq) in MeOH (2.6 mL) was added 3 M HCl in MeOH (2.3 mL, 7.0 mmol, 10.0 eq) dropwise at 0° C. The reaction mixture was allowed to slowly warm to room temperature. After 1 hour, the reaction mixture was concentrated under reduced pressure. The resulting crude material was dissolved in EtOAc (5 mL) and sat. NaHCO₃ (5 mL) and stirred vigorously for 10 minutes. The aqueous phase was extracted with EtOAc (3×2 mL) to give N-((5-chloro-6-hydroxy-1H-indol-2-yl)methyl-d₂)acetamide, which was used without further purification. LC-MS (ESI) m/z calcd for C₁₁H₉D₂ClN₂O₂: 240.1; found: 240.7 [M+H]⁺.

### Example 1: (Compound 1)

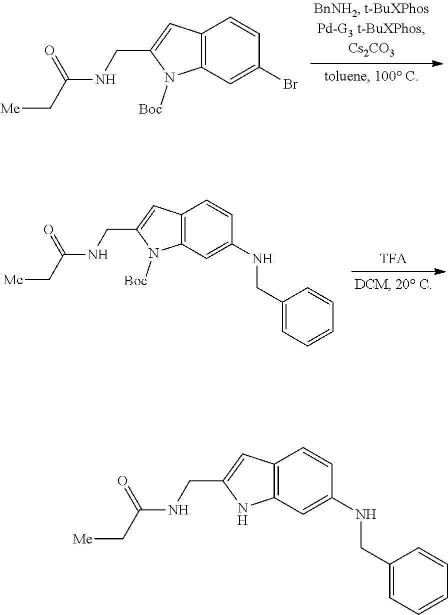 [SMILES: CCC(=O)NCc1cc2ccc(Br)cc2n1C(=O)OC(C)(C)C ; CCC(=O)NCc1cc2ccc(NCc3ccccc3)cc2n1C(=O)OC(C)(C)C ; CCC(=O)NCc1cc2ccc(NCc3ccccc3)cc2[nH]1]

### Step 1: tert-butyl 6-(benzylamino)-2-(propionamidomethyl)-1H-indole-1-carboxylate

Three reactions were carried out in parallel. To a solution of tert-butyl 6-bromo-2-(propionamidomethyl)-1H-indole-1-carboxylate (200 mg, 525 μmol, 1.00 eq) and phenylmethanamine (114 μL, 1.05 mmol, 2.00 eq) in toluene (2 mL) were added Cs₂CO₃ (342 mg, 1.05 mmol, 2.00 eq), [2-(2-aminophenyl)phenyl]-methylsulfonyloxy-palladium; di-tert-butyl-[2-(2,4,6-triisopropylphenyl)phenyl]phosphane (t-BuXPhos Pd-G3, 41.7 mg, 52.5 μmol, 0.10 eq) and di-tert-butyl-[2-(2,4,6-triisopropylphenyl)phenyl]phosphane (t-BuXPhos, 22.3 mg, 52.5 μmol, 0.10 eq) under N₂, and the mixture was stirred at 100° C. for 12 h. The three parallel reactions were combined for work up. After cooling to room temperature, the combined reaction mixture was poured into water (5 mL) and stirred for 5 min. The aqueous phase was extracted with ethyl acetate (2×10 mL). The combined organic phase was washed with brine (2×10 mL), dried over anhydrous Na₂SO₄, filtered, and concentrated under reduced pressure. The residue was purified by column chromatography to give tert-butyl 6-(benzylamino)-2-(propionamidomethyl)-1H-indole-1-carboxylate. LC-MS (ESI) m/z calcd for C₂₄H₂₉N₃O₃: 407.2; found 408.3 [M+H]⁺.

### Step 2: N-((6-(benzylamino)-1H-indol-2-yl)methyl)propionamide

To a solution of tert-butyl 6-(benzylamino)-2-(propionamidomethyl)-1H-indole-1-carboxylate (130 mg, 319 μmol, 1.00 eq) in DCM (1 mL) was added TFA (1.00 mL, 13.5 mmol, 42.3 eq), and the mixture was stirred at 20° C. for 2 h. The mixture was then concentrated under reduced pressure and purified by prep-HPLC to give N-((6-(benzylamino)-1H-indol-2-yl)methyl)propionamide (Compound 1). LC-MS (ESI) m/z calcd for C₁₉H₂₁N₃O: 307.2; found 308.1 [M+H]⁺. ¹H NMR (400 MHz, DMSO-d₆) δ 11.20 (s, 1H), 8.30 (t, J=5.6 Hz, 1H), 7.51 (d, J=8.0 Hz, 1H), 7.48-7.44 (m, 2H), 7.40 (s, 4H), 7.01 (d, J=5.6 Hz, 1H), 6.27 (s, 1H), 4.53 (s, 2H), 4.38 (d, J=5.6 Hz, 2H), 2.18-2.12 (m, 2H), 1.03 (t, J=8.0 Hz, 3H).

The following compounds in Table T-1 were synthesized using procedures similar to Compound 1 using the appropriate starting materials.

TABLE T-1

| | | | Exact | LCMS, |
|---|---|---|---|---|
| Compound | | | mass | found |
| No. | Structure | IUPAC name | (g/mol) | [M + H]⁺ |
| 2 | 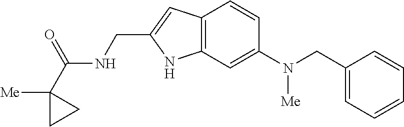 [SMILES: CN(Cc1ccccc1)c1ccc2cc(CNC(=O)C3(C)CC3)[nH]c2c1] | N-((6- (benzyl(methyl)amino)- 1H-indol-2-yl)methyl)- 1-methylcyclopropane- 1-carboxamide | 347.20 | 348.2 |

### Example 2: (Compound 3)

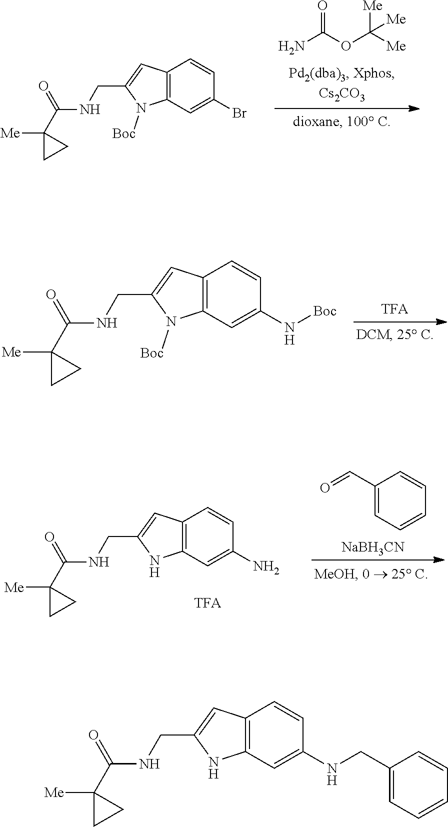

### Step 1: tert-butyl 6-((tert-butoxycarbonyl)amino)-2-((1-methylcyclopropanecarboxamido)methyl)-1H-indole-1-carboxylate

To a solution of tert-butyl 6-bromo-2-((1-methylcyclopropanecarboxamido)methyl)-1H-indole-1-carboxylate (500 mg, 1.23 mmol, 1.00 eq) and tert-butyl carbamate (216 mg, 1.84 mmol, 1.50 eq) in dioxane (6 mL) were added Cs₂CO₃ (780 mg, 2.46 mmol, 2.00 eq), XPhos (117 mg, 246 μmol, 0.20 eq) and Pd₂(dba)₃ (225 mg, 246 μmol, 0.20 eq) under N₂, and the resulting mixture was heated to 100° C. and stirred for 12 h. The reaction mixture was allowed to cool to room temperature, then was poured into water (10 mL) and stirred for 2 min. The aqueous phase was extracted with ethyl acetate (3×15 mL). The combined organic phase was washed with brine (2×15 mL), dried over anhydrous Na₂SO₄, filtered, and concentrated under reduced pressure. The residue was purified by column chromatography to give tert-butyl 6-((tert-butoxycarbonyl)amino)-2-((1-methylcyclopropanecarboxamido)methyl)-1H-indole-1-carboxylate. LC-MS (ESI) m/z calcd for C₂₄H₃₃N₃O₅: 443.2; found 444.3 [M+H]⁺.

### Step 2: N-((6-amino-1H-indol-2-yl)methyl)-1-methylcyclopropanecarboxamide 2,2,2-trifluoroacetic acid salt

To a solution of tert-butyl 6-((tert-butoxycarbonyl)amino)-2-((1-methylcyclopropanecarboxamido)methyl)-1H-indole-1-carboxylate (500 mg, 1.13 mmol, 1.00 eq) in DCM (0.5 mL) was added TFA (5.00 mL, 67.5 mmol, 59.9 eq), and the mixture was stirred at 25° C. for 2 h. The mixture was then concentrated under reduced pressure to give N-((6-amino-1H-indol-2-yl)methyl)-1-methylcyclopropanecarboxamide trifluoroacetic acid salt. LC-MS (ESI) m/z calcd for C₁₄H₁₇N₃O: 243.1; found 244.1 [M+H]⁺.

### Step 3: N-((6-(benzylamino)-1H-indol-2-yl)methyl)-1-methylcyclopropanecarboxamide

A solution of N-((6-amino-1H-indol-2-yl)methyl)-1-methylcyclopropanecarboxamide (200 mg, 288 μmol, 35% purity, 1.00 eq) and benzaldehyde (26.2 μL, 259 μmol, 0.90 eq) in MeOH (2 mL) was added NaBH₃CN (54 mg, 863 μmol, 3.00 eq) at 0° C., then the mixture was stirred at 25° C. for 1 h. The mixture was then poured into water (5 mL). The aqueous phase was extracted with ethyl acetate (10 mL×3). The combined organic phase was washed with brine (10 mL×2), dried over anhydrous Na₂SO₄, filtered, and concentrated under reduced pressure. The residue was purified by prep-HPLC to give N-((6-(benzylamino)-1H-indol-2-yl)methyl)-1-methylcyclopropanecarboxamide (Compound 3). LC-MS (ESI) m/z calcd for C₂₁H₂₃N₃O: 333.2; found 334.2 [M+H]⁺. ¹H NMR (400 MHz, DMSO-d₆) δ 11.05 (s, 1H), 8.04 (s, 1H), 7.46-7.45 (m, 3H), 7.38-7.35 (m, 4H), 6.94 (s, 1H), 6.21 (s, 1H), 4.50-4.48 (m, 2H), 4.38 (d, J=5.6 Hz, 2H), 1.29 (s, 3H), 1.00-0.97 (m, 2H), 0.54-0.52 (m, 2H).

The following compounds in Table T-2 were synthesized using procedures similar to Compound 3 using the appropriate starting materials.

TABLE T-2

| | | | Exact | LCMS, |
|---|---|---|---|---|
| Compound | | | mass | found |
| No. | Structure | IUPAC name | (g/mol) | [M + H]⁺ |
| 4 | 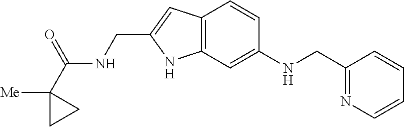 [SMILES: CC1(C(=O)NCc2cc3ccc(NCc4ccccn4)cc3[nH]2)CC1] | 1-methyl-N-((6- ((pyridin-2- ylmethyl)amino)-1H- indol-2- yl)methyl)cyclopropane- 1-carboxamide | 334.18 | 335.2 |

### Example 3: (Compound 5)

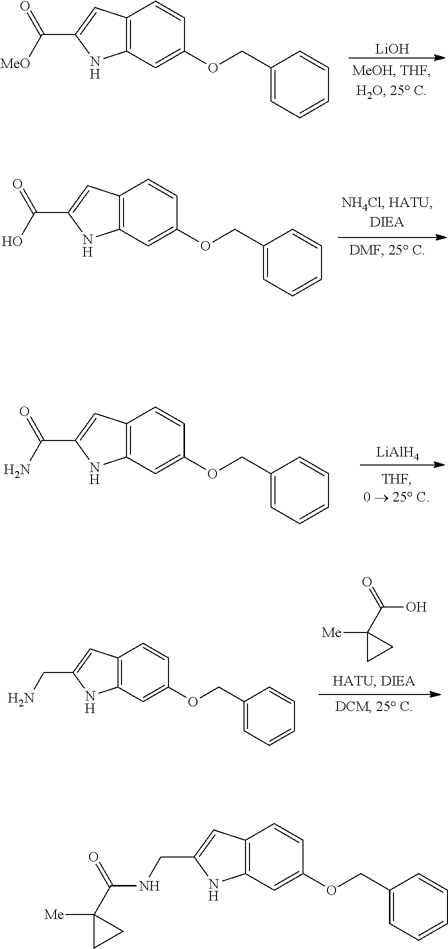

### Step 1: 6-(benzyloxy)-1H-indole-2-carboxylic acid

To a solution of methyl 6-(benzyloxy)-1H-indole-2-carboxylate (500 mg, 1.78 mmol, 1.00 eq) in MeOH (20 mL) were added LiOH·H₂O (298 mg, 7.11 mmol, 4.00 eq), H₂O (10 mL) and THF (10 mL). The mixture was stirred at 25° C. for 16 h. The reaction mixture was concentrated under reduced pressure, then was diluted with H₂O (20 mL), adjusted to pH 5 by addition of 1M aq. HCl, and filtered to give 6-(benzyloxy)-1H-indole-2-carboxylic acid. LC-MS (ESI) m/z: calcd for C₁₆H₁₃NO₃: 267.1; found 268.1 [M+H]⁺.

### Step 2: 6-(benzyloxy)-1H-indole-2-carboxamide

Two reactions were carried out in parallel. To a solution of 6-(benzyloxy)-1H-indole-2-carboxylic acid (200 mg, 748 μmol, 1.00 eq) in DMF (2 mL) were added NH₄Cl (120 mg, 2.24 mmol, 3.00 eq), HATU (370 mg, 973 μmol, 1.30 eq) and DIEA (391 μL, 2.24 mmol, 3.00 eq). The resulting mixture was stirred at 25° C. for 16 h. The reaction mixture was quenched by addition of H₂O (20 mL) and extracted with EtOAc (3×15 mL). The combined organic layers were washed with brine (10 mL), dried over Na₂SO₄, filtered, and concentrated under reduced pressure. Purification by column chromatography gave 6-(benzyloxy)-1H-indole-2-carboxamide. LC-MS (ESI) m/z calcd for C₁₆H₁₄N₂O₂: 266.1; found 267.2 [M+H]⁺.

### Step 3: (6-(benzyloxy)-1H-indol-2-yl)methanamine

To a solution of 6-(benzyloxy)-1H-indole-2-carboxamide (350 mg, 1.31 mmol, 1.00 eq) in THF (7 mL) was added LiAlH₄ (399 mg, 10.5 mmol, 8.00 eq) at 0° C. under N₂ atmosphere. The mixture was stirred at 25° C. for 16 h. The reaction mixture was quenched by dropwise addition of water (0.5 mL), 15% aqueous NaOH (0.5 mL), and water (1.5 mL). The resulting mixture was filtered and washed with THF (2×20 mL). The filtrate was concentrated under reduced pressure and purified by column chromatography to give (6-(benzyloxy)-1H-indol-2-yl)methanamine. LC-MS (ESI) m/z calcd for C₁₆H₁₆N₂O: 252.1; found 253.0 [M+H]⁺.

### Step 4: N-((6-(benzyloxy)-1H-indol-2-yl)methyl)-1-methylcyclopropanecarboxamide

To a solution of (6-(benzyloxy)-1H-indol-2-yl)methanamine (110 mg, 436 μmol, 1.00 eq) in DCM (1 mL) were added 1-methylcyclopropanecarboxylic acid (52 mg, 523 μmol, 1.20 eq), DIEA (380 μL, 2.18 mmol, 5.00 eq) and HATU (216 mg, 567 μmol, 1.30 eq). The mixture was stirred at 25° C. for 1 h, then was concentrated under reduced pressure. Purification by prep-HPLC gave N-((6-(benzyloxy)-1H-indol-2-yl)methyl)-1-methylcyclopropanecarboxamide (Compound 5). LC-MS (ESI) m/z calcd for C₂₁H₂₂N₂O₂: 334.2; found 335.2 [M+H]⁺. ¹H NMR (400 MHz, DMSO-d₆) δ 10.55 (s, 1H), 7.97 (t, J=5.6 Hz, 1H), 7.45 (d, J=5.6 Hz, 2H), 7.40-7.36 (m, 2H), 7.31-7.29 (m, 2H), 6.94 (d, J=2.0 Hz, 1H), 6.69-6.66 (m, 1H), 6.10 (s, 1H), 5.08 (s, 2H), 4.34 (d, J=6.0 Hz, 2H), 1.28 (s, 3H), 1.00-0.98 (m, 2H), 0.53-0.51 (m, 2H).

### Example 4: (Compound 6)

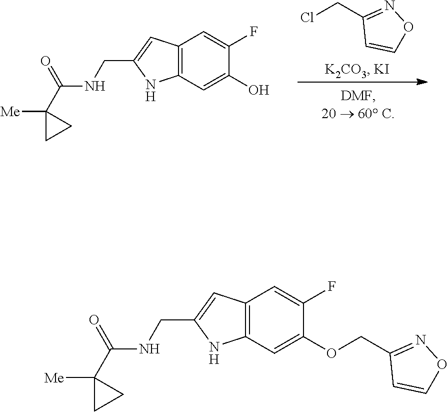

### Step 1: N-((5-fluoro-6-(isoxazol-3-ylmethoxy)-1H-indol-2-yl)methyl)-1-methylcyclopropane-1-carboxamide

To a solution of N-((5-fluoro-6-hydroxy-1H-indol-2-yl)methyl)-1-methylcyclopropane-1-carboxamide (100 mg, 381 μmol, 1.00 eq) in DMF (2 mL) were added 3-(chloromethyl)isoxazole (44.8 mg, 291 μmol, 0.80 eq), KI (63.2 mg, 381 μmol, 1.00 eq) and K₂CO₃ (105 mg, 762 μmol, 2.00 eq). The mixture was heated to 60° C. and stirred for 2 h. After allowing the mixture to cool to room temperature, the residue was poured into water (10 mL) and stirred for 3 min. The aqueous phase was extracted with ethyl acetate (3×10 mL). The combined organic phase was washed with brine (20 mL), dried over anhydrous sodium sulfate, filtered, and concentrated under reduced pressure. Purification by prep-HPLC gave N-((5-fluoro-6-(isoxazol-3-ylmethoxy)-1H-indol-2-yl)methyl)-1-methylcyclopropane-1-carboxamide (Compound 6). LC-MS (ESI) m/z calcd for C₁₈H₁₈FN₃O₃: 343.1; found 344.1 [M+H]⁺. ¹H NMR (400 MHz, DMSO-d₆) δ 10.73 (s, 1H), 8.94 (d, J=1.6 Hz, 1H), 8.01-7.98 (m, 1H), 7.26-7.22 (m, 1H), 7.15 (d, J=7.2 Hz, 1H), 6.70 (s, 1H), 6.12 (s, 1H), 5.25 (s, 2H), 4.34 (d, J=5.6 Hz, 2H), 1.28 (s, 3H), 1.00-0.97 (m, 2H), 0.53-0.51 (m, 2H).

The following compounds in Table T-3 were synthesized using procedures similar to Compound 6 using the appropriate starting materials.

TABLE T-3

| | | | Exact | LCMS, |
|---|---|---|---|---|
| Compound | | | mass | found |
| No. | Structure | IUPAC name | (g/mol) | [M + H]⁺ |
| 7 | 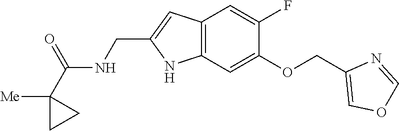 [SMILES: CC1(C(=O)NCc2cc3cc(F)c(OCc4cocn4)cc3[nH]2)CC1] | N-((5-fluoro-6-(oxazol- 4-ylmethoxy)-1H-indol- 2-yl)methyl)-1- methylcyclopropane-1- carboxamide | 343.13 | 344.1 |
| 8 | 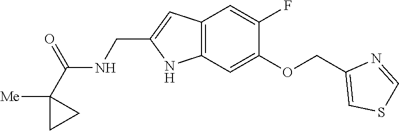 [SMILES: CC1(C(=O)NCc2cc3cc(F)c(OCc4cscn4)cc3[nH]2)CC1] | N-((5-fluoro-6-(thiazol- 4-ylmethoxy)-1H-indol- 2-yl)methyl)-1- methylcyclopropane-1- carboxamide | 359.11 | 360.1 |
| 9 | 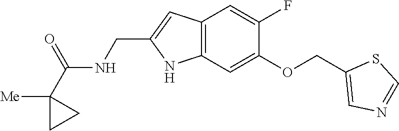 [SMILES: CC1(C(=O)NCc2cc3cc(F)c(OCc4cncs4)cc3[nH]2)CC1] | N-((5-fluoro-6-(thiazol- 5-ylmethoxy)-1H-indol- 2-yl)methyl)-1- methylcyclopropane-1- carboxamide | 359.11 | 360.0 |
| 10 | 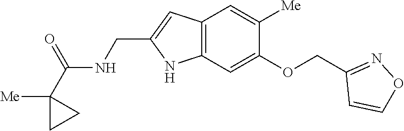 [SMILES: Cc1cc2cc(CNC(=O)C3(C)CC3)[nH]c2cc1OCc1ccon1] | N-((6-(isoxazol-3- ylmethoxy)-5-methyl- 1H-indol-2-yl)methyl)- 1-methylcyclopropane- 1-carboxamide | 339.16 | 340.2 |
| 11 | 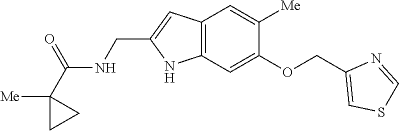 [SMILES: Cc1cc2cc(CNC(=O)C3(C)CC3)[nH]c2cc1OCc1cscn1] | 1-methyl-N-((5-methyl- 6-(thiazol-4- ylmethoxy)-1H-indol-2- yl)methyl)cyclopropane- 1-carboxamide | 355.14 | 356.1 |
| 12 | 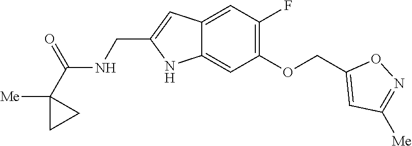 [SMILES: Cc1cc(COc2cc3[nH]c(CNC(=O)C4(C)CC4)cc3cc2F)on1] | N-((5-fluoro-6-((3- methylisoxazol-5- yl)methoxy)-1H-indol- 2-yl)methyl)-1- methylcyclopropane-1- carboxamide | 357.15 | 358.1 |
| 13 | 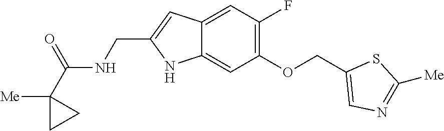 [SMILES: Cc1ncc(COc2cc3[nH]c(CNC(=O)C4(C)CC4)cc3cc2F)s1] | N-((5-fluoro-6-((2- methylthiazol-5- yl)methoxy)-1H-indol- 2-yl)methyl)-1- methylcyclopropane-1- carboxamide | 373.13 | 374.1 |
| 14 | 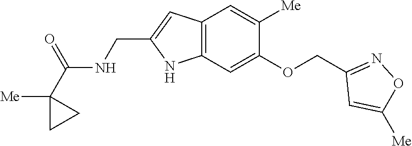 [SMILES: Cc1cc(COc2cc3[nH]c(CNC(=O)C4(C)CC4)cc3cc2C)no1] | 1-methyl-N-((5-methyl- 6-((5-methylisoxazol-3- yl)methoxy)-1H-indol- 2- yl)methyl)cyclopropane- 1-carboxamide | 353.17 | 354.2 |
| 15 | 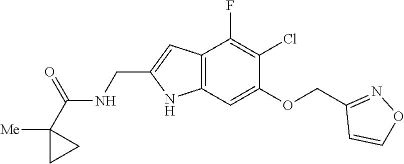 [SMILES: CC1(C(=O)NCc2cc3c(F)c(Cl)c(OCc4ccon4)cc3[nH]2)CC1] | N-((5-chloro-4-fluoro-6- (isoxazol-3-ylmethoxy)- 1H-indol-2-yl)methyl)- 1-methylcyclopropane- 1-carboxamide | 377.09 | 378.0 |
| 16 | 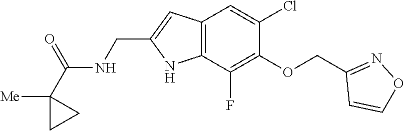 [SMILES: CC1(C(=O)NCc2cc3cc(Cl)c(OCc4ccon4)c(F)c3[nH]2)CC1] | N-((5-chloro-7-fluoro-6- (isoxazol-3-ylmethoxy)- 1H-indol-2-yl)methyl)- 1-methylcyclopropane- 1-carboxamide | 377.09 | 378.1 |
| 17 | 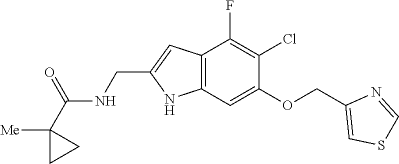 [SMILES: CC1(C(=O)NCc2cc3c(F)c(Cl)c(OCc4cscn4)cc3[nH]2)CC1] | N-((5-chloro-4-fluoro-6- (thiazol-4-ylmethoxy)- 1H-indol-2-yl)methyl)- 1-methylcyclopropane- 1-carboxamide | 393.07 | 393.9 |
| 18 | 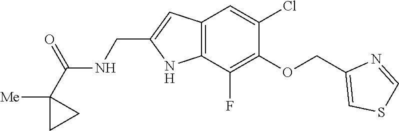 [SMILES: CC1(C(=O)NCc2cc3cc(Cl)c(OCc4cscn4)c(F)c3[nH]2)CC1] | N-((5-chloro-7-fluoro-6- (thiazol-4-ylmethoxy)- 1H-indol-2-yl)methyl)- 1-methylcyclopropane- 1-carboxamide | 393.07 | 394.0 |
| 19 | 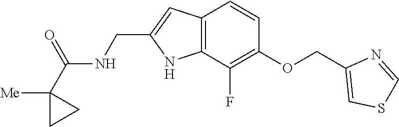 [SMILES: CC1(C(=O)NCc2cc3ccc(OCc4cscn4)c(F)c3[nH]2)CC1] | N-((7-fluoro-6-(thiazol- 4-ylmethoxy)-1H-indol- 2-yl)methyl)-1- methylcyclopropane-1- carboxamide | 359.11 | 360.1 |
| 201 | 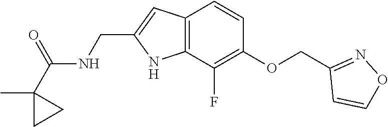 [SMILES: CC1(C(=O)NCc2cc3ccc(OCc4ccon4)c(F)c3[nH]2)CC1] | N-((7-fluoro-6- (isoxazol-3-ylmethoxy)- 1H-indol-2-yl)methyl)- 1-methylcyclopropane- 1-carboxamide | 343.13 | 344.2 |
| 318 | 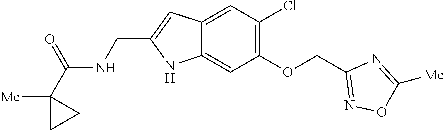 [SMILES: Cc1nc(COc2cc3[nH]c(CNC(=O)C4(C)CC4)cc3cc2Cl)no1] | N-((5-chloro-6-((5- methyl-1,2,4-oxadiazol- 3-yl)methoxy)-1H- indol-2-yl)methyl)-1- methylcyclopropane-1- carboxamide | 374.11 | 375.2 |
| 319 | 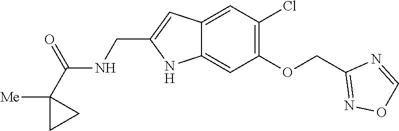 [SMILES: CC1(C(=O)NCc2cc3cc(Cl)c(OCc4ncon4)cc3[nH]2)CC1] | N-((6-((1,2,4-oxadiazol- 3-yl)methoxy)-5-chloro- 1H-indol-2-yl)methyl)- 1-methylcyclopropane- 1-carboxamide | 360.10 | 361.2 |
| 320 | 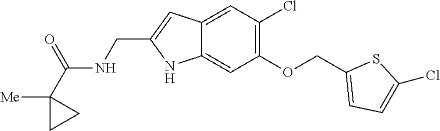 [SMILES: CC1(C(=O)NCc2cc3cc(Cl)c(OCc4ccc(Cl)s4)cc3[nH]2)CC1] | N-((5-chloro-6-((5- chlorothiophen-2- yl)methoxy)-1H-indol- 2-yl)methyl)-1- methylcyclopropane-1- carboxamide | 408.05 | 409.2 |
| 321 | 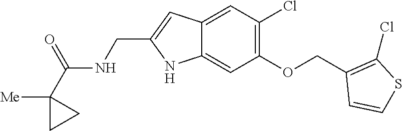 [SMILES: CC1(C(=O)NCc2cc3cc(Cl)c(OCc4ccsc4Cl)cc3[nH]2)CC1] | N-((5-chloro-6-((2- chlorothiophen-3- yl)methoxy)-1H-indol- 2-yl)methyl)-1- methylcyclopropane-1- carboxamide | 408.05 | 409.2 |
| 322 | 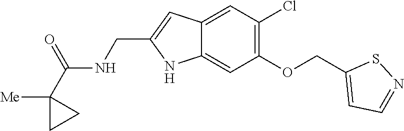 [SMILES: CC1(C(=O)NCc2cc3cc(Cl)c(OCc4ccns4)cc3[nH]2)CC1] | N-((5-chloro-6- (isothiazol-5- ylmethoxy)-1H-indol-2- yl)methyl)-1- methylcyclopropane-1- carboxamide | 375.08 | 376.2 |
| 323 | 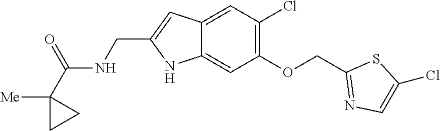 [SMILES: CC1(C(=O)NCc2cc3cc(Cl)c(OCc4ncc(Cl)s4)cc3[nH]2)CC1] | N-((5-chloro-6-((5- chlorothiazol-2- yl)methoxy)-1H-indol- 2-yl)methyl)-1- methylcyclopropane-1- carboxamide | 409.04 | 410.2 |
| 324 | 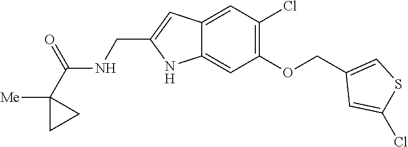 [SMILES: CC1(C(=O)NCc2cc3cc(Cl)c(OCc4csc(Cl)c4)cc3[nH]2)CC1] | N-((5-chloro-6-((5- chlorothiophen-3- yl)methoxy)-1H-indol- 2-yl)methyl)-1- methylcyclopropane-1- carboxamide | 408.05 | 409.1 |
| 325 | 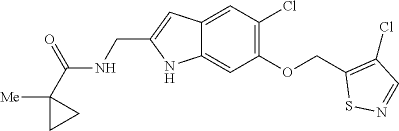 [SMILES: CC1(C(=O)NCc2cc3cc(Cl)c(OCc4sncc4Cl)cc3[nH]2)CC1] | N-((5-chloro-6-((4- chloroisothiazol-5- yl)methoxy)-1H-indol- 2-yl)methyl)-1- methylcyclopropane-1- carboxamide | 409.04 | 410.1 |

### Example 5: (Compound 20)

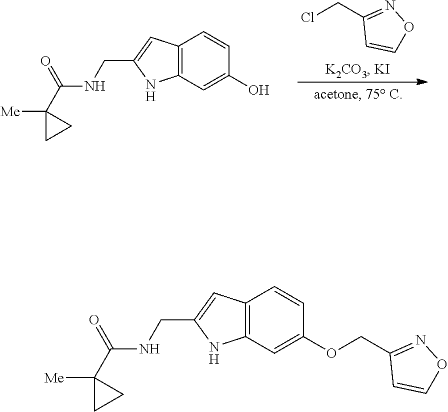

### Step 1: N-((6-(isoxazol-3-ylmethoxy)-1H-indol-2-yl)methyl)-1-methylcyclopropane-1-carboxamide

To a solution of N-((6-hydroxy-1H-indol-2-yl)methyl)-1-methylcyclopropane-1-carboxamide (100 mg, 409 μmol, 1.00 eq) and 3-(chloromethyl)isoxazole (49 mg, 417 μmol, 1.02 eq) in acetone (5 mL) were added Cs₂CO₃ (133 mg, 409 μmol, 1.00 eq) and KI (5 mg, 28.6 μmol, 0.07 eq), and the mixture was heated to 75° C. and stirred for 16 h. The reaction mixture was then poured into water (10 mL) and stirred for 10 min. The aqueous phase was extracted with ethyl acetate (2×20 mL). The combined organic phase was washed with brine (2×20 mL), dried over anhydrous Na₂SO₄, filtered, and concentrated under reduced pressure. Purification by prep-HPLC gave N-((6-(isoxazol-3-ylmethoxy)-1H-indol-2-yl)methyl)-1-methylcyclopropane-1-carboxamide (Compound 20). LC-MS (ESI) m/z calcd for C₁₈H₁₉N₃O₃: 325.14; found 326.1 [M+H]⁺. ¹H NMR (400 MHz, DMSO-d₆) δ 10.62 (s, 1H), 8.93 (d, J=1.6 Hz, 1H), 7.97 (m, 1H), 7.32 (d, J=8.8 Hz, 1H), 6.98 (d, J=2.0 Hz, 1H), 6.69-6.67 (m, 2H), 6.12 (s, 1H), 5.19 (s, 2H), 4.35 (d, J=5.6 Hz, 2H), 1.29 (s, 3H), 1.00-0.98 (m, 2H), 0.54-0.51 (m, 2H).

The following compounds in Table T-4 were synthesized using procedures similar to Compound 20 using the appropriate starting materials.

TABLE T-4

| | | | Exact | LCMS, |
|---|---|---|---|---|
| Cmpd | | | mass | found |
| No. | Structure | IUPAC name | (g/mol) | [M + H]⁺ |
| 21 | 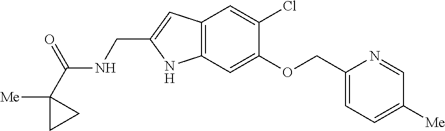 [SMILES: Cc1ccc(COc2cc3[nH]c(CNC(=O)C4(C)CC4)cc3cc2Cl)nc1] | N-((5-chloro-6-((5- methylpyridin-2-yl)methoxy)- 1H-indol-2-yl)methyl)-1- methylcyclopropane-1- carboxamide | 383.14 | 384.0 |

| | | | | |
|---|---|---|---|---|
| 22 | 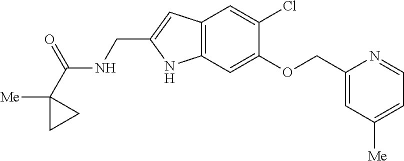 [SMILES: Cc1ccnc(COc2cc3[nH]c(CNC(=O)C4(C)CC4)cc3cc2Cl)c1] | N-((5-chloro-6-((4- methylpyridin-2-yl)methoxy)- 1H-indol-2-yl)methyl)-1- methylcyclopropane-1- carboxamide | 383.14 | 384.2 |

| | | | | |
|---|---|---|---|---|
| 23 | 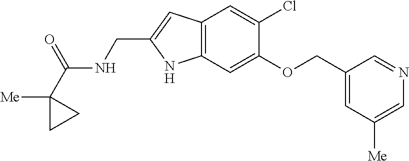 [SMILES: Cc1cncc(COc2cc3[nH]c(CNC(=O)C4(C)CC4)cc3cc2Cl)c1] | N-((5-chloro-6-((5- methylpyridin-3-yl)methoxy)- 1H-indol-2-yl)methyl)-1- methylcyclopropane-1- carboxamide | 383.14 | 384.0 |

| | | | | |
|---|---|---|---|---|
| 24 | 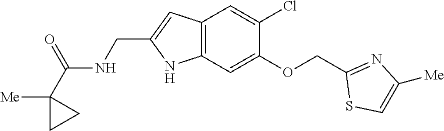 [SMILES: Cc1csc(COc2cc3[nH]c(CNC(=O)C4(C)CC4)cc3cc2Cl)n1] | N-((5-chloro-6-((4- methylthiazol-2-yl)methoxy)- 1H-indol-2-yl)methyl)-1- methylcyclopropane-1- carboxamide | 389.10 | 389.9 |

| | | | | |
|---|---|---|---|---|
| 25 | 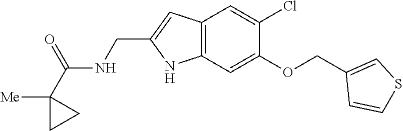 [SMILES: CC1(C(=O)NCc2cc3cc(Cl)c(OCc4ccsc4)cc3[nH]2)CC1] | N-((5-chloro-6-(thiophen-3- ylmethoxy)-1H-indol-2- yl)methyl)-1- methylcyclopropane-1- carboxamide | 374.09 | 374.9 |

| | | | | |
|---|---|---|---|---|
| 26 | 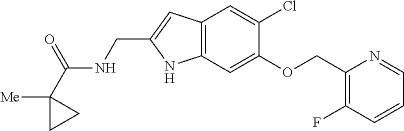 [SMILES: CC1(C(=O)NCc2cc3cc(Cl)c(OCc4ncccc4F)cc3[nH]2)CC1] | N-((5-chloro-6-((3- fluoropyridin-2-yl)methoxy)- 1H-indol-2-yl)methyl)-1- methylcyclopropane-1- carboxamide | 387.11 | 388.1 |
| 27 | 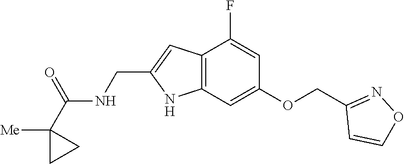 [SMILES: CC1(C(=O)NCc2cc3c(F)cc(OCc4ccon4)cc3[nH]2)CC1] | N-((4-fluoro-6-(isoxazol-3- ylmethoxy)-1H-indol-2- yl)methyl)-1- methylcyclopropane-1- carboxamide | 343.13 | 344.0 |
| 28 | 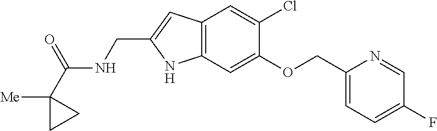 [SMILES: CC1(C(=O)NCc2cc3cc(Cl)c(OCc4ccc(F)cn4)cc3[nH]2)CC1] | N-((5-chloro-6-((5- fluoropyridin-2-yl)methoxy)- 1H-indol-2-yl)methyl)-1- methylcyclopropane-1- carboxamide | 387.11 | 388.1 |
| 29 | 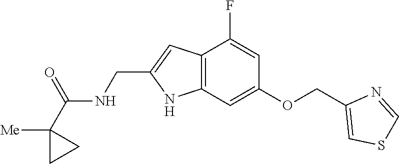 [SMILES: CC1(C(=O)NCc2cc3c(F)cc(OCc4cscn4)cc3[nH]2)CC1] | N-((4-fluoro-6-(thiazol-4- ylmethoxy)-1H-indol-2- yl)methyl)-1- methylcyclopropane-1- carboxamide | 359.11 | 360.0 |
| 30 | 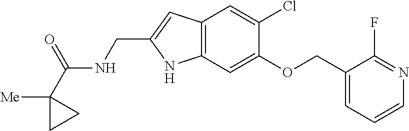 [SMILES: CC1(C(=O)NCc2cc3cc(Cl)c(OCc4cccnc4F)cc3[nH]2)CC1] | N-((5-chloro-6-((2- fluoropyridin-3-yl)methoxy)- 1H-indol-2-yl)methyl)-1- methylcyclopropane-1- carboxamide | 387.11 | 388.0 |
| 31 | 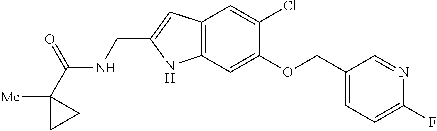 [SMILES: CC1(C(=O)NCc2cc3cc(Cl)c(OCc4ccc(F)nc4)cc3[nH]2)CC1] | N-((5-chloro-6-((6- fluoropyridin-3-yl)methoxy)- 1H-indol-2-yl)methyl)-1- methylcyclopropane-1- carboxamide | 387.11 | 388.1 |
| 32 | 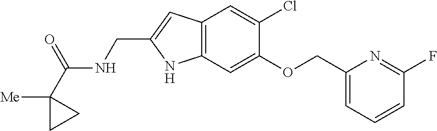 [SMILES: CC1(C(=O)NCc2cc3cc(Cl)c(OCc4cccc(F)n4)cc3[nH]2)CC1] | N-((5-chloro-6-((6- fluoropyridin-2-yl)methoxy)- 1H-indol-2-yl)methyl)-1- methylcyclopropane-1- carboxamide | 387.11 | 388.1 |
| 33 | 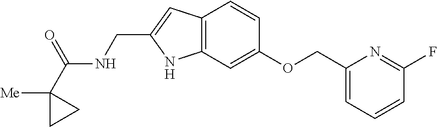 [SMILES: CC1(C(=O)NCc2cc3ccc(OCc4cccc(F)n4)cc3[nH]2)CC1] | N-((6-((6-fluoropyridin-2- yl)methoxy)-1H-indol-2- yl)methyl)-1- methylcyclopropane-1- carboxamide | 353.15 | 354.2 |
| 34 | 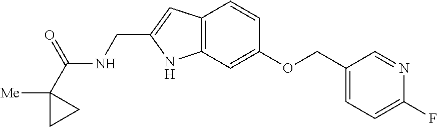 [SMILES: CC1(C(=O)NCc2cc3ccc(OCc4ccc(F)nc4)cc3[nH]2)CC1] | N-((6-((6-fluoropyridin-3- yl)methoxy)-1H-indol-2- yl)methyl)-1- methylcyclopropane-1- carboxamide | 353.15 | 354.1 |
| 35 | 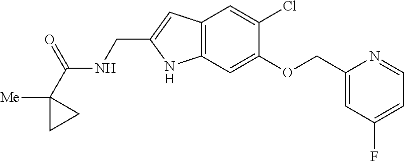 [SMILES: CC1(C(=O)NCc2cc3cc(Cl)c(OCc4cc(F)ccn4)cc3[nH]2)CC1] | N-((5-chloro-6-((4- fluoropyridin-2-yl)methoxy)- 1H-indol-2-yl)methyl)-1- methylcyclopropane-1- carboxamide | 387.12 | 388.1 |
| 36 | 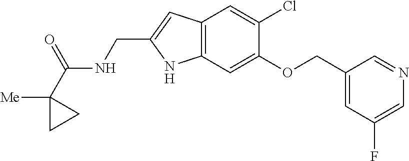 [SMILES: CC1(C(=O)NCc2cc3cc(Cl)c(OCc4cncc(F)c4)cc3[nH]2)CC1] | N-((5-chloro-6-((5- fluoropyridin-3-yl)methoxy)- 1H-indol-2-yl)methyl)-1- methylcyclopropane-1- carboxamide | 387.12 | 388.2 |
| 37 | 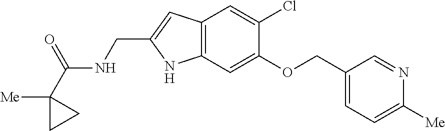 [SMILES: Cc1ccc(COc2cc3[nH]c(CNC(=O)C4(C)CC4)cc3cc2Cl)cn1] | N-((5-chloro-6-((6- methylpyridin-3-yl)methoxy)- 1H-indol-2-yl)methyl)-1- methylcyclopropane-1- carboxamide | 383.14 | 384.1 |
| 38 | 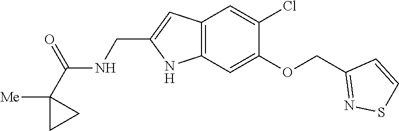 [SMILES: CC1(C(=O)NCc2cc3cc(Cl)c(OCc4ccsn4)cc3[nH]2)CC1] | N-((5-chloro-6-(isothiazol-3- ylmethoxy)-1H-indol-2- yl)methyl)-1- methylcyclopropane-1- carboxamide | 375.08 | 376.0 |
| 39 | 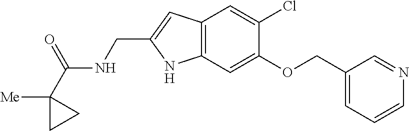 [SMILES: CC1(C(=O)NCc2cc3cc(Cl)c(OCc4cccnc4)cc3[nH]2)CC1] | N-((5-chloro-6-(pyridin-3- ylmethoxy)-1H-indol-2- yl)methyl)-1- methylcyclopropane-1- carboxamide | 369.12 | 369.8 |
| 40 | 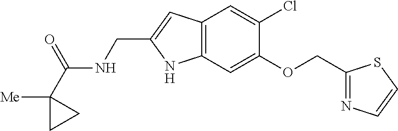 [SMILES: CC1(C(=O)NCc2cc3cc(Cl)c(OCc4nccs4)cc3[nH]2)CC1] | N-((5-chloro-6-(thiazol-2- ylmethoxy)-1H-indol-2- yl)methyl)-1- methylcyclopropane-1- carboxamide | 375.08 | 375.8 |
| 41 | 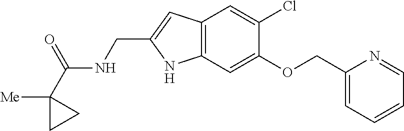 [SMILES: CC1(C(=O)NCc2cc3cc(Cl)c(OCc4ccccn4)cc3[nH]2)CC1] | N-((5-chloro-6-(pyridin-2- ylmethoxy)-1H-indol-2- yl)methyl)-1- methylcyclopropane-1- carboxamide | 369.12 | 369.8 |
| 42 | 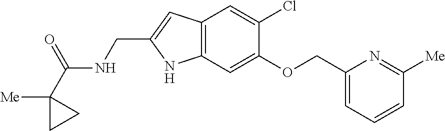 [SMILES: Cc1cccc(COc2cc3[nH]c(CNC(=O)C4(C)CC4)cc3cc2Cl)n1] | N-((5-chloro-6-((6- methylpyridin-2-yl)methoxy)- 1H-indol-2-yl)methyl)-1- methylcyclopropane-1- carboxamide | 383.14 | 383.8 |
| 43 | 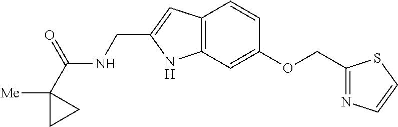 [SMILES: CC1(C(=O)NCc2cc3ccc(OCc4nccs4)cc3[nH]2)CC1] | 1-methyl-N-((6-(thiazol-2- ylmethoxy)-1H-indol-2- yl)methyl)cyclopropane-1- carboxamide | 341.12 | 342.1 |
| 44 | 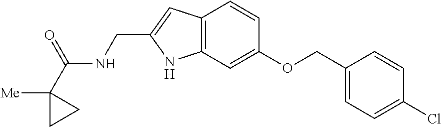 [SMILES: CC1(C(=O)NCc2cc3ccc(OCc4ccc(Cl)cc4)cc3[nH]2)CC1] | N-((6-((4-chlorobenzyl)oxy)- 1H-indol-2-yl)methyl)-1- methylcyclopropane-1- carboxamide | 368.13 | 369.0 |
| 45 | 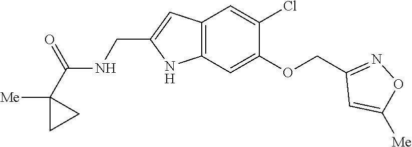 [SMILES: Cc1cc(COc2cc3[nH]c(CNC(=O)C4(C)CC4)cc3cc2Cl)no1] | N-((5-chloro-6-((5- methylisoxazol-3-yl)methoxy)- 1H-indol-2-yl)methyl)-1- methylcyclopropane-1- carboxamide | 373.12 | 374.1 |
| 46 | 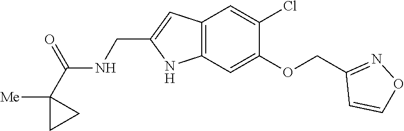 [SMILES: CC1(C(=O)NCc2cc3cc(Cl)c(OCc4ccon4)cc3[nH]2)CC1] | N-((5-chloro-6-(isoxazol-3- ylmethoxy)-1H-indol-2- yl)methyl)-1- methylcyclopropane-1- carboxamide | 359.10 | 360.0 |
| 47 | 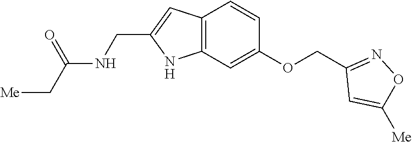 [SMILES: CCC(=O)NCc1cc2ccc(OCc3cc(C)on3)cc2[nH]1] | N-((6-((5-methylisoxazol-3- yl)methoxy)-1H-indol-2- yl)methyl)propionamide | 313.14 | 314.0 |
| 48 | 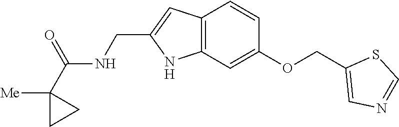 [SMILES: CC1(C(=O)NCc2cc3ccc(OCc4cncs4)cc3[nH]2)CC1] | 1-methyl-N-((6-(thiazol-5- ylmethoxy)-1H-indol-2- yl)methyl)cyclopropane-1- carboxamide | 341.12 | 342.0 |
| 49 | 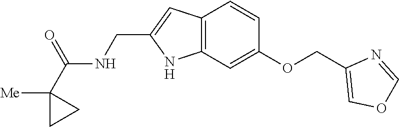 [SMILES: CC1(C(=O)NCc2cc3ccc(OCc4cocn4)cc3[nH]2)CC1] | 1-methyl-N-((6-(oxazol-4- ylmethoxy)-1H-indol-2- yl)methyl)cyclopropane-1- carboxamide | 325.14 | 326.1 |
| 50 | 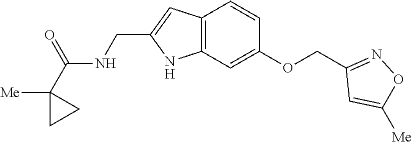 [SMILES: Cc1cc(COc2ccc3cc(CNC(=O)C4(C)CC4)[nH]c3c2)no1] | 1-methyl-N-((6-((5- methylisoxazol-3-yl)methoxy)- 1H-indol-2- yl)methyl)cyclopropane-1- carboxamide | 339.16 | 340.2 |
| 51 | 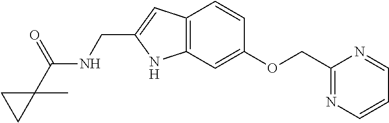 [SMILES: CC1(C(=O)NCc2cc3ccc(OCc4ncccn4)cc3[nH]2)CC1] | 1-methyl-N-((6-(pyrimidin-2- ylmethoxy)-1H-indol-2- yl)methyl)cyclopropane-1- carboxamide | 336.16 | 337.3 |
| 52 | 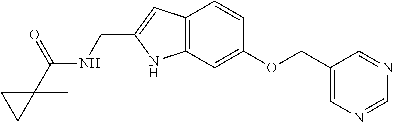 [SMILES: CC1(C(=O)NCc2cc3ccc(OCc4cncnc4)cc3[nH]2)CC1] | 1-methyl-N-((6-(pyrimidin-5- ylmethoxy)-1H-indol-2- yl)methyl)cyclopropane-1- carboxamide | 336.16 | 337.2 |
| 53 | 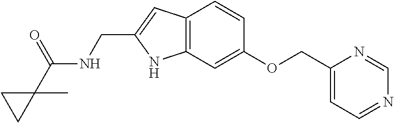 [SMILES: CC1(C(=O)NCc2cc3ccc(OCc4ccncn4)cc3[nH]2)CC1] | 1-methyl-N-((6-(pyrimidin-4- ylmethoxy)-1H-indol-2- yl)methyl)cyclopropane-1- carboxamide | 336.16 | 337.2 |
| 54 | 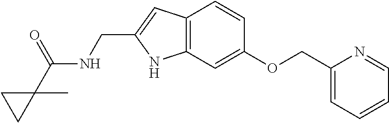 [SMILES: CC1(C(=O)NCc2cc3ccc(OCc4ccccn4)cc3[nH]2)CC1] | 1-methyl-N-((6-(pyridin-2- ylmethoxy)-1H-indol-2- yl)methyl)cyclopropane-1- carboxamide | 335.12 | 335.7 |
| 55 | 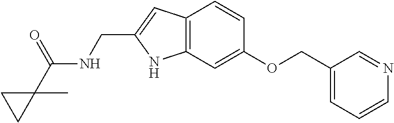 [SMILES: CC1(C(=O)NCc2cc3ccc(OCc4cccnc4)cc3[nH]2)CC1] | 1-methyl-N-((6-(pyridin-3- ylmethoxy)-1H-indol-2- yl)methyl)cyclopropane-1- carboxamide | 335.16 | 336.13 |
| 56 | 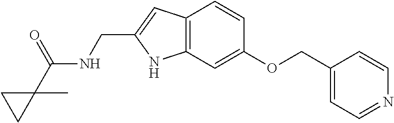 [SMILES: CC1(C(=O)NCc2cc3ccc(OCc4ccncc4)cc3[nH]2)CC1] | 1-methyl-N-((6-(pyridin-4- ylmethoxy)-1H-indol-2- yl)methyl)cyclopropane-1- carboxamide | 335.16 | 336.13 |
| 326 | 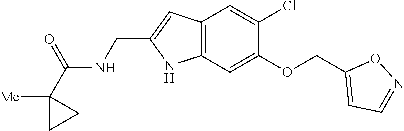 [SMILES: CC1(C(=O)NCc2cc3cc(Cl)c(OCc4ccno4)cc3[nH]2)CC1] | N-((5-chloro-6-(isoxazol-5- ylmethoxy)-1H-indol-2- yl)methyl)-1- methylcyclopropane-1- carboxamide | 359.10 | 360.5 |
| 327 | 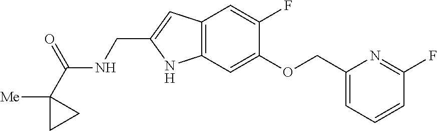 [SMILES: CC1(C(=O)NCc2cc3cc(F)c(OCc4cccc(F)n4)cc3[nH]2)CC1] | N-((5-fluoro-6-((6- fluoropyridin-2-yl)methoxy)- 1H-indol-2-yl)methyl)-1- methylcyclopropane-1- carboxamide | 371.14 | 372.2 |
| 328 | 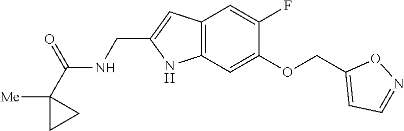 [SMILES: CC1(C(=O)NCc2cc3cc(F)c(OCc4ccno4)cc3[nH]2)CC1] | N-((5-fluoro-6-(isoxazol-5- ylmethoxy)-1H-indol-2- yl)methyl)-1- methylcyclopropane-1- carboxamide | 343.13 | 344.2 |
| 329 | 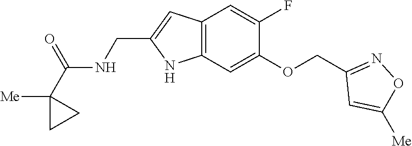 [SMILES: Cc1cc(COc2cc3[nH]c(CNC(=O)C4(C)CC4)cc3cc2F)no1] | N-((5-fluoro-6-((5- methylisoxazol-3-yl)methoxy)- 1H-indol-2-yl)methyl)-1- methylcyclopropane-1- carboxamide | 357.15 | 358.2 |
| 330 | 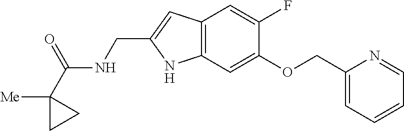 [SMILES: CC1(C(=O)NCc2cc3cc(F)c(OCc4ccccn4)cc3[nH]2)CC1] | N-((5-fluoro-6-(pyridin-2- ylmethoxy)-1H-indol-2- yl)methyl)-1- methylcyclopropane-1- carboxamide | 353.15 | 353.2 |
| 331 | 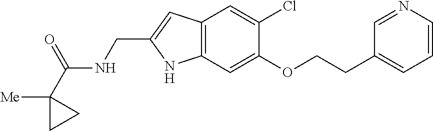 [SMILES: CC1(C(=O)NCc2cc3cc(Cl)c(OCCc4cccnc4)cc3[nH]2)CC1] | N-((5-chloro-6-(2-(pyridin-3- yl)ethoxy)-1H-indol-2- yl)methyl)-1- methylcyclopropane-1- carboxamide | 383.14 | 384.2 |
| 332 | 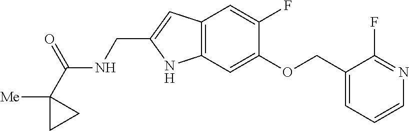 [SMILES: CC1(C(=O)NCc2cc3cc(F)c(OCc4cccnc4F)cc3[nH]2)CC1] | N-((5-fluoro-6-((2- fluoropyridin-3-yl)methoxy)- 1H-indol-2-yl)methyl)-1- methylcyclopropane-1- carboxamide | 371.14 | 372.2 |
| 333 | 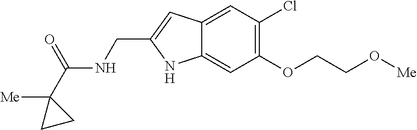 [SMILES: COCCOc1cc2[nH]c(CNC(=O)C3(C)CC3)cc2cc1Cl] | N-((5-chloro-6-(2- methoxyethoxy)-1H-indol-2- yl)methyl)-1- methylcyclopropane-1- carboxamide | 336.12 | 337.2 |
| 334 | 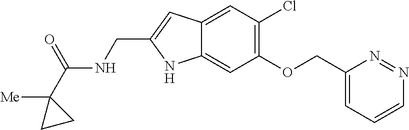 [SMILES: CC1(C(=O)NCc2cc3cc(Cl)c(OCc4cccnn4)cc3[nH]2)CC1] | N-((5-chloro-6-(pyridazin-3- ylmethoxy)-1H-indol-2- yl)methyl)-1- methylcyclopropane-1- carboxamide | 370.12 | 371.2 |
| 335 | 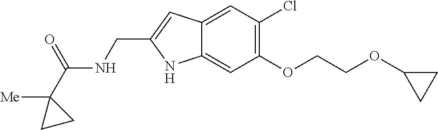 [SMILES: CC1(C(=O)NCc2cc3cc(Cl)c(OCCOC4CC4)cc3[nH]2)CC1] | N-((5-chloro-6-(2- cyclopropoxyethoxy)-1H- indol-2-yl)methyl)-1- methylcyclopropane-1- carboxamide | 362.14 | 385.2 [M + Na]⁺ |
| 336 | 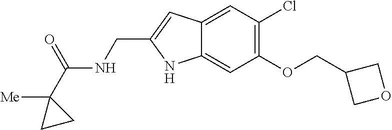 [SMILES: CC1(C(=O)NCc2cc3cc(Cl)c(OCC4COC4)cc3[nH]2)CC1] | N-((5-chloro-6-(oxetan-3- ylmethoxy)-1H-indol-2- yl)methyl)-1- methylcyclopropane-1- carboxamide | 348.12 | 348.9 |
| 337 | 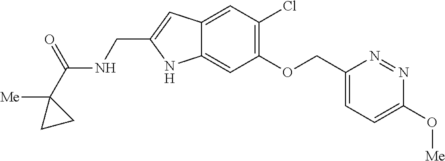 [SMILES: COc1ccc(COc2cc3[nH]c(CNC(=O)C4(C)CC4)cc3cc2Cl)nn1] | N-((5-chloro-6-((6- methoxypyridazin-3- yl)methoxy)-1H-indol-2- yl)methyl)-1- methylcyclopropane-1- carboxamide | 400.13 | 401.2 |
| 338 | 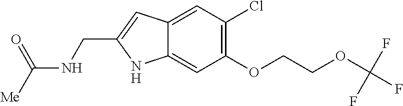 [SMILES: CC(=O)NCc1cc2cc(Cl)c(OCCOC(F)(F)F)cc2[nH]1] | N-((5-chloro-6-(2- (trifluoromethoxy)ethoxy)-1H- indol-2-yl)methyl)acetamide | 350.06 | 292.0 [M − C₂H₄NO + H]⁺ |
| 339 | 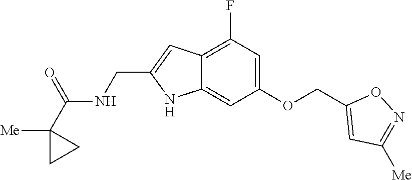 [SMILES: Cc1cc(COc2cc(F)c3cc(CNC(=O)C4(C)CC4)[nH]c3c2)on1] | N-((4-fluoro-6-((3- methylisoxazol-5-yl)methoxy)- 1H-indol-2-yl)methyl)-1- methylcyclopropane-1- carboxamide | 357.15 | 358.3 |
| 340 | 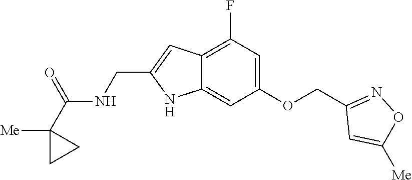 [SMILES: Cc1cc(COc2cc(F)c3cc(CNC(=O)C4(C)CC4)[nH]c3c2)no1] | N-((4-fluoro-6-((5- methylisoxazol-3-yl)methoxy)- 1H-indol-2-yl)methyl)-1- methylcyclopropane-1- carboxamide | 357.15 | 358.3 |
| 341 | 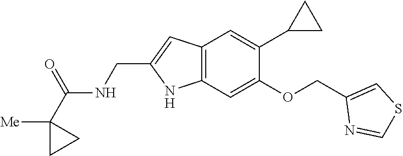 [SMILES: CC1(C(=O)NCc2cc3cc(C4CC4)c(OCc4cscn4)cc3[nH]2)CC1] | N-((5-cyclopropyl-6-(thiazol-4- ylmethoxy)-1H-indol-2- yl)methyl)-1- methylcyclopropane-1- carboxamide | 381.15 | 382.2 |
| 342 | 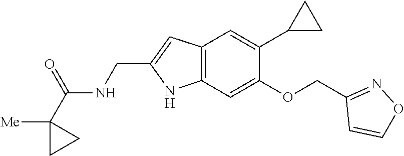 [SMILES: CC1(C(=O)NCc2cc3cc(C4CC4)c(OCc4ccon4)cc3[nH]2)CC1] | N-((5-cyclopropyl-6-(isoxazol- 3-ylmethoxy)-1H-indol-2- yl)methyl)-1- methylcyclopropane-1- carboxamide | 365.17 | 366.1 |
| 343 | 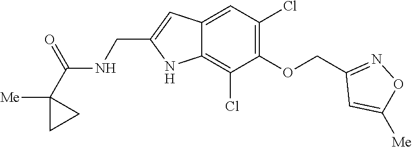 [SMILES: Cc1cc(COc2c(Cl)cc3cc(CNC(=O)C4(C)CC4)[nH]c3c2Cl)no1] | N-((5,7-dichloro-6-((5- methylisoxazol-3-yl)methoxy)- 1H-indol-2-yl)methyl)-1- methylcyclopropane-1- carboxamide | 407.08 | 408.1 |
| 344 | 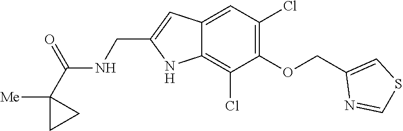 [SMILES: CC1(C(=O)NCc2cc3cc(Cl)c(OCc4cscn4)c(Cl)c3[nH]2)CC1] | N-((5,7-dichloro-6-(thiazol-4- ylmethoxy)-1H-indol-2- yl)methyl)-1- methylcyclopropane-1- carboxamide | 409.04 | 410.0 |
| 345 | 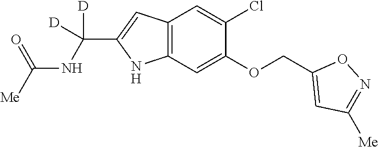 [SMILES: [2H]C([2H])(NC(C)=O)c1cc2cc(Cl)c(OCc3cc(C)no3)cc2[nH]1] | N-((5-chloro-6-((3- methylisoxazol-5-yl)methoxy)- 1H-indol-2-yl)methyl- d2)acetamide | 335.10 | 336.1 |
| 346 | 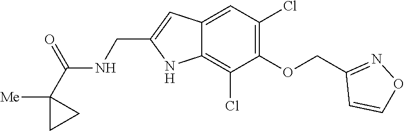 [SMILES: CC1(C(=O)NCc2cc3cc(Cl)c(OCc4ccon4)c(Cl)c3[nH]2)CC1] | N-((5,7-dichloro-6-(isoxazol-3- ylmethoxy)-1H-indol-2- yl)methyl)-1- methylcyclopropane-1- carboxamide | 393.06 | 394.1 |
| 347 | 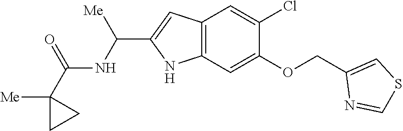 [SMILES: CC(NC(=O)C1(C)CC1)c1cc2cc(Cl)c(OCc3cscn3)cc2[nH]1] | N-(1-(5-chloro-6-(thiazol-4- ylmethoxy)-1H-indol-2- yl)ethyl)-1- methylcyclopropane-1- carboxamide | 389.10 | 390.2 |
| 348 | 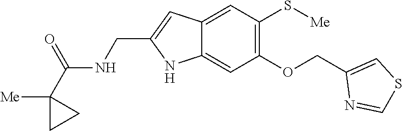 [SMILES: CSc1cc2cc(CNC(=O)C3(C)CC3)[nH]c2cc1OCc1cscn1] | 1-methyl-N-((5-(methylthio)-6- (thiazol-4-ylmethoxy)-1H- indol-2- yl)methyl)cyclopropane-1- carboxamide | 387.11 | 388.1 |

### Example 6: (Compound 57)

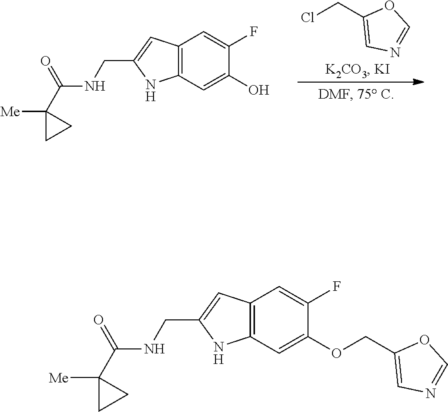

### Step 1: N-((5-fluoro-6-(oxazol-5-ylmethoxy)-1H-indol-2-yl)methyl)-1-methylcyclopropane-1-carboxamide

To a solution of N-((5-fluoro-6-hydroxy-1H-indol-2-yl)methyl)-1-methylcyclopropane-1-carboxamide (100 mg, 381 μmol, 1.00 eq) in DMF (2 mL) were added K₂CO₃ (158 mg, 1.14 mmol, 3.00 eq), KI (63.0 mg, 381 μmol, 1.00 eq) and 5-(chloromethyl)oxazole (76 mg, 495 μmol, 1.30 eq). The mixture was heated to 75° C. and stirred for 2 h. The reaction mixture was allowed to cool to room temperature, then was poured into water (10 mL). The aqueous phase was extracted with ethyl acetate (5×10 mL), and the combined organic phases were washed with brine (10 mL), dried over anhydrous sodium sulfate, filtered, and concentrated under reduced pressure. Purification by prep-HPLC gave N-((5-fluoro-6-(oxazol-5-ylmethoxy)-1H-indol-2-yl)methyl)-1-methylcyclopropane-1-carboxamide (Compound 57). LC-MS (ESI) m/z calcd for C₁₈H₁₈FN₃O₃: 343.13; found 344.1 [M+H]⁺. ¹H NMR (400 MHz, DMSO-d₆) δ 9.11 (s, 1H), 7.89 (t, J=6.4 Hz, 1H), 7.21 (d, J=12.0 Hz, 1H), 7.15 (s, 1H), 6.96 (d, J=1.6 Hz, 1H), 6.37 (t, J=5.6 Hz, 1H), 6.23 (s, 1H), 5.14 (s, 2H), 4.45 (d, J=2.8 Hz, 2H), 1.56 (s, 3H), 1.32-1.25 (m, 2H), 0.66-0.63 (m, 2H).

### Example 7: (Compound 58)

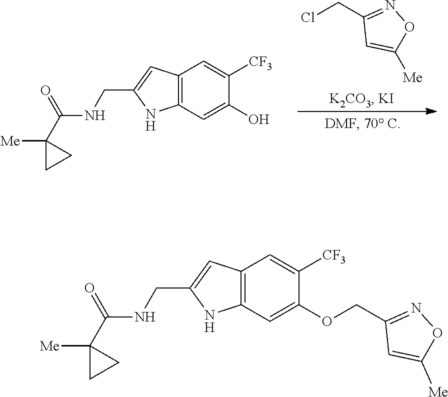

### Step 1: 1-methyl-N-((6-((5-methylisoxazol-3-yl)methoxy)-5-(trifluoromethyl)-1H-indol-2-yl)methyl)cyclopropanecarboxamide

To a solution of N-((6-hydroxy-5-(trifluoromethyl)-1H-indol-2-yl)methyl)-1-methylcyclopropanecarboxamide (80 mg, 256 μmol, 1.00 eq) and 3-(chloromethyl)-5-methylisoxazole (34 mg, 256 μmol, 1.00 eq) in DMF (1 mL) were added Cs₂CO₃ (125 mg, 384 μmol, 1.50 eq) and KI (5 mg, 25.6 μmol, 0.10 eq). The resulting mixture was heated to 70° C. and stirred for 2 h. The mixture was allowed to cool to room temperature, then was poured into water (10 mL) and stirred for 5 min. The aqueous phase was extracted with ethyl acetate (2×20 mL). The combined organic phase was washed with brine (2×20 mL), dried over anhydrous Na₂SO₄, filtered, and concentrated under reduced pressure. The mixture was purified by prep-HPLC to give 1-methyl-N-((6-((5-methylisoxazol-3-yl)methoxy)-5-(trifluoromethyl)-1H-indol-2-yl)methyl)cyclopropanecarboxamide (Compound 58). LC-MS (ESI) m/z calcd for C₂₀H₂₀F₃N₃O₃: 407.15; found 406.0 [M−H]-. ¹H NMR (400 MHz, DMSO-d₆) δ 11.06 (s, 1H), 8.05 (t, J=5.6 Hz, 1H), 7.74 (s, 1H), 7.22 (s, 1H), 6.27 (d, J=1.0 Hz, 1H), 6.24 (s, 1H), 5.25 (s, 2H), 4.37 (d, J=5.6 Hz, 2H), 2.41 (s, 3H), 1.29 (s, 3H), 1.01-0.99 (m, 2H), 0.55-0.53 (m, 2H).

The following compounds in Table T-5 were synthesized using procedures similar to Compound 58 using the appropriate starting materials.

TABLE T-5

| | | | Exact | LCMS, |
|---|---|---|---|---|
| Cmpd | | | mass | found |
| No. | Structure | IUPAC name | (g/mol) | [M + H]⁺ |
| 59 | 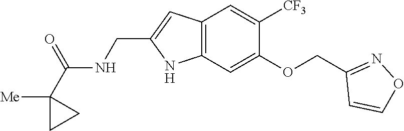 [SMILES: CC1(C(=O)NCc2cc3cc(C(F)(F)F)c(OCc4ccon4)cc3[nH]2)CC1] | N-((6-(isoxazol-3-ylmethoxy)- 5-(trifluoromethyl)-1H-indol-2- yl)methyl)-1- methylcyclopropane-1- carboxamide | 393.13 | 392.0 [M − H]⁻ |
| 60 | 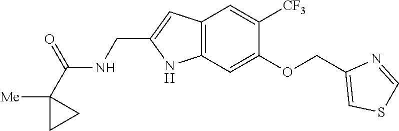 [SMILES: CC1(C(=O)NCc2cc3cc(C(F)(F)F)c(OCc4cscn4)cc3[nH]2)CC1] | N-((6-(thiazol-4-ylmethoxy)-5- (trifluoromethyl)-1H-indol-2- yl)methyl)-1-methyl- cyclopropane-1-carboxamide | 409.11 | 408.0 [M − H]⁻ |
| 61 | 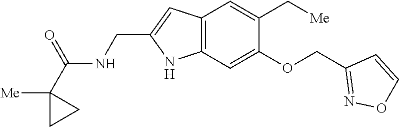 [SMILES: CCc1cc2cc(CNC(=O)C3(C)CC3)[nH]c2cc1OCc1ccon1] | N-((5-ethyl-6-(isoxazol-3- ylmethoxy)-1H-indol-2- yl)methyl)-1- methylcyclopropane-1- carboxamide | 353.17 | 354.0 |
| 202 | 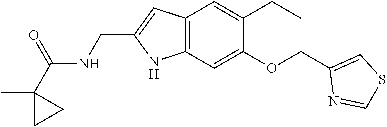 [SMILES: CCc1cc2cc(CNC(=O)C3(C)CC3)[nH]c2cc1OCc1cscn1] | N-((5-ethyl-6-(thiazol-4- ylmethoxy)-1H-indol-2- yl)methyl)-1- methylcyclopropane-1- carboxamide | 369.15 | 370.0 |

### Example 8: (Compound 62)

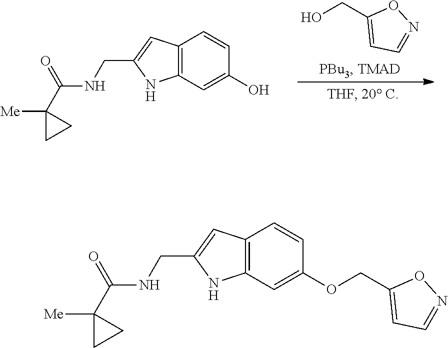

### Step 1: N-((6-(isoxazol-5-ylmethoxy)-1H-indol-2-yl)methyl)-1-methylcyclopropanecarboxamide

To a solution of N-((6-hydroxy-1H-indol-2-yl)methyl)-1-methylcyclopropanecarboxamide (150 mg, 614 μmol, 1.00 eq) and isoxazol-5-ylmethanol (212 mg, 2.15 mmol, 3.50 eq) in THF (3 mL) were added tributylphosphine (455 μL, 1.84 mmol, 3.00 eq) and TMAD (317 mg, 1.84 mmol, 3.00 eq) under N₂. The resulting mixture was stirred for 16 h, then was quenched by addition of water (10 mL) and extracted with ethyl acetate (2×5 mL). The combined organic phase was washed with brine (10 mL), dried over anhydrous sodium sulfate, filtered, and concentrated under reduced pressure. Purification by prep-TLC gave N-((6-(isoxazol-5-ylmethoxy)-H-indol-2-yl)methyl)-1-methylcyclopropanecarboxamide (Compound 62). LC-MS (ESI) m/z calcd for C₁₈H₉N₃O₃: 325.14; found 326.1 [M+H]⁺. ¹H NMR (400 MHz, DMSO-d₆) δ 10.62 (s, 1H), 8.57 (s, 1H), 7.98 (t, J=5.6 Hz, 1H), 7.32 (d, J=8.4 Hz, 1H), 6.98 (d, J=2.0 Hz, 1H), 6.70-6.67 (m, 1H), 6.59 (s, 1H), 6.12 (s, 1H), 5.26 (s, 2H), 4.35 (d, J=5.6 Hz, 2H), 1.29 (s, 3H), 1.00-0.98 (m, 2H), 0.54-0.51 (m, 2H).

The following compounds in Table T-6 were synthesized using procedures similar to Compound 62 using the appropriate starting materials.

TABLE T-6

| Com- | | | Exact | LCMS, |
|---|---|---|---|---|
| pound | | | mass | found |
| No. | Structure | IUPAC name | (g/mol) | [M + H]⁺ |
| 63 | 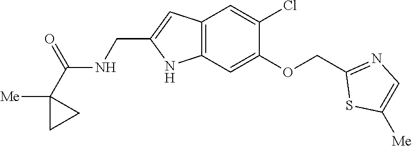 [SMILES: Cc1cnc(COc2cc3[nH]c(CNC(=O)C4(C)CC4)cc3cc2Cl)s1] | N-((5-chloro-6-((5- methylthiazol-2- yl)methoxy)-1H-indol-2- yl)methyl)-1- methylcyclopropane-1- carboxamide | 389.10 | 390.1 |
| 64 | 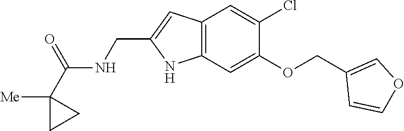 [SMILES: CC1(C(=O)NCc2cc3cc(Cl)c(OCc4ccoc4)cc3[nH]2)CC1] | N-((5-chloro-6-(furan-3- ylmethoxy)-1H-indol-2- yl)methyl)-1- methylcyclopropane-1- carboxamide | 358.11 | 359.1 |
| 65 | 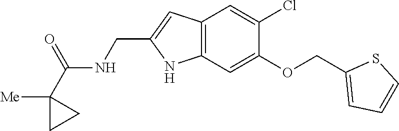 [SMILES: CC1(C(=O)NCc2cc3cc(Cl)c(OCc4cccs4)cc3[nH]2)CC1] | N-((5-chloro-6-(thiophen-2- ylmethoxy)-1H-indol-2- yl)methyl)-1- methylcyclopropane-1- carboxamide | 374.09 | 375.0 |
| 66 | 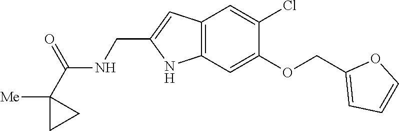 [SMILES: CC1(C(=O)NCc2cc3cc(Cl)c(OCc4ccco4)cc3[nH]2)CC1] | N-((5-chloro-6-(furan-2- ylmethoxy)-1H-indol-2- yl)methyl)-1- methylcyclopropane-1- carboxamide | 358.11 | 359.0 |
| 67 | 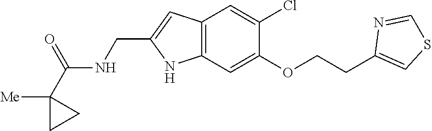 [SMILES: CC1(C(=O)NCc2cc3cc(Cl)c(OCCc4cscn4)cc3[nH]2)CC1] | N-((5-chloro-6-(2-(thiazol- 4-yl)ethoxy)-1H-indol-2- yl)methyl)-1- methylcyclopropane-1- carboxamide | 389.10 | 390.0 |
| 68 | 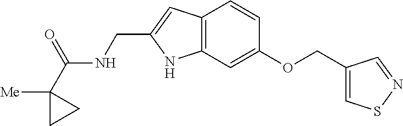 [SMILES: CC1(C(=O)NCc2cc3ccc(OCc4cnsc4)cc3[nH]2)CC1] | N-((6-(isothiazol-4- ylmethoxy)-1H-indol-2- yl)methyl)-1- methylcyclopropane-1- carboxamide | 341.12 | 342.1 |
| 69 | 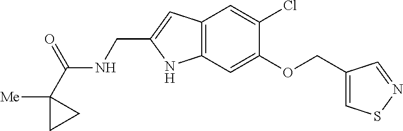 [SMILES: CC1(C(=O)NCc2cc3cc(Cl)c(OCc4cnsc4)cc3[nH]2)CC1] | N-((5-chloro-6-(isothiazol- 4-ylmethoxy)-1H-indol-2- yl)methyl)-1- methylcyclopropane-1- carboxamide | 375.08 | 376.1 |
| 70 | 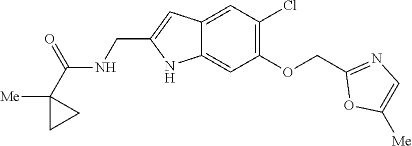 [SMILES: Cc1cnc(COc2cc3[nH]c(CNC(=O)C4(C)CC4)cc3cc2Cl)o1] | N-((5-chloro-6-((5- methyloxazol-2- yl)methoxy)-1H-indol-2- yl)methyl)-1- methylcyclopropane-1- carboxamide | 373.12 | 374.1 |
| 71 | 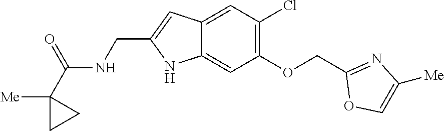 [SMILES: Cc1coc(COc2cc3[nH]c(CNC(=O)C4(C)CC4)cc3cc2Cl)n1] | N-((5-chloro-6-((4- methyloxazol-2- yl)methoxy)-1H-indol-2- yl)methyl)-1- methylcyclopropane-1- carboxamide | 373.12 | 374.1 |
| 72 | 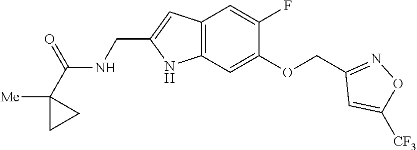 [SMILES: CC1(C(=O)NCc2cc3cc(F)c(OCc4cc(C(F)(F)F)on4)cc3[nH]2)CC1] | N-((5-fluoro-6-((5- (trifluoromethyl)isoxazol-3- yl)methoxy)-1H-indol-2- yl)methyl)-1- methylcyclopropane-1- carboxamide | 411.12 | 412.0 |
| 73 | 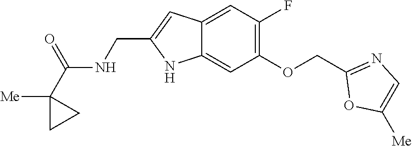 [SMILES: Cc1cnc(COc2cc3[nH]c(CNC(=O)C4(C)CC4)cc3cc2F)o1] | N-((5-fluoro-6-((5- methyloxazol-2- yl)methoxy)-1H-indol-2- yl)methyl)-1- methylcyclopropane-1- carboxamide | 357.15 | 358.0 |
| 74 | 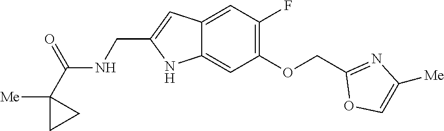 [SMILES: Cc1coc(COc2cc3[nH]c(CNC(=O)C4(C)CC4)cc3cc2F)n1] | N-((5-fluoro-6-((5- methyloxazol-2- yl)methoxy)-1H-indol-2- yl)methyl)-1- methylcyclopropane-1- carboxamide | 357.15 | 358.2 |
| 349 | 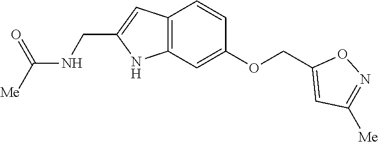 [SMILES: CC(=O)NCc1cc2ccc(OCc3cc(C)no3)cc2[nH]1] | N-((6-((3-methylisoxazol-5- yl)methoxy)-1H-indol-2- yl)methyl)acetamide | 299.13 | 300.1 |
| 350 | 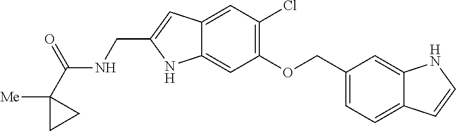 [SMILES: CC1(C(=O)NCc2cc3cc(Cl)c(OCc4ccc5cc[nH]c5c4)cc3[nH]2)CC1] | N-((6-((1H-indol-6- yl)methoxy)-5-chloro-1H- indol-2-yl)methyl)-1- methylcyclopropane-1- carboxamide | 407.01 | 408.8 |
| 351 | 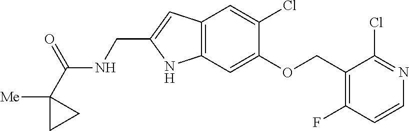 [SMILES: CC1(C(=O)NCc2cc3cc(Cl)c(OCc4c(F)ccnc4Cl)cc3[nH]2)CC1] | N-((5-chloro-6-((2-chloro-4- fluoropyridin-3- yl)methoxy)-1H-indol-2- yl)methyl)-1- methylcyclopropane-1- carboxamide | 421.08 | 422.1 |
| 352 | 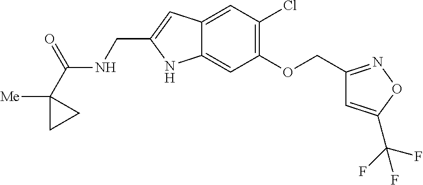 [SMILES: CC1(C(=O)NCc2cc3cc(Cl)c(OCc4cc(C(F)(F)F)on4)cc3[nH]2)CC1] | N-((5-chloro-6-((5- (trifluoromethyl)isoxazol-3- yl)methoxy)-1H-indol-2- yl)methyl)-1- methylcyclopropane-1- carboxamide | 427.09 | 428.1 |
| 353 | 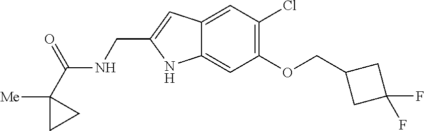 [SMILES: CC1(C(=O)NCc2cc3cc(Cl)c(OCC4CC(F)(F)C4)cc3[nH]2)CC1] | N-((5-chloro-6-((3,3- difluorocyclobutyl)methoxy)- 1H-indol-2-yl)methyl)-1- methylcyclopropane-1- carboxamide | 382.13 | 383.1 |
| 354 | 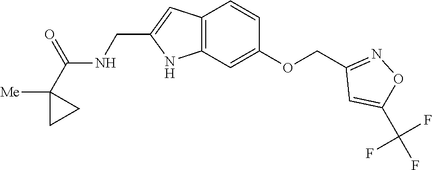 [SMILES: CC1(C(=O)NCc2cc3ccc(OCc4cc(C(F)(F)F)on4)cc3[nH]2)CC1] | 1-methyl-N-((6-((5- (trifluoromethyl)isoxazol-3- yl)methoxy)-1H-indol-2- yl)methyl)cyclopropane-1- carboxamide | 393.13 | 394.0 |
| 355 | 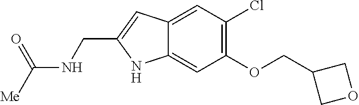 [SMILES: CC(=O)NCc1cc2cc(Cl)c(OCC3COC3)cc2[nH]1] | N-((5-chloro-6-(oxetan-3- ylmethoxy)-1H-indol-2- yl)methyl)acetamide | 308.09 | 309.0 |
| 356 | 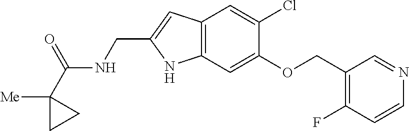 [SMILES: CC1(C(=O)NCc2cc3cc(Cl)c(OCc4cnccc4F)cc3[nH]2)CC1] | N-((5-chloro-6-((4- fluoropyridin-3- yl)methoxy)-1H-indol-2- yl)methyl)-1- methylcyclopropane-1- carboxamide | 387.11 | 388.3 |

### Example 9: (Compound 75)

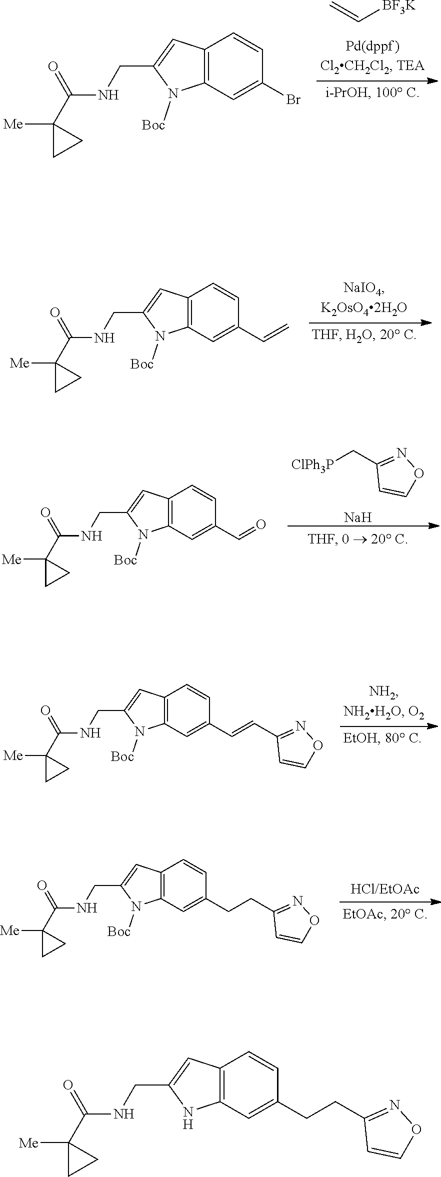

### Step 1: tert-butyl 2-((1-methylcyclopropanecarboxamido)methyl)-6-vinyl-1H-indole-1-carboxylate

To a solution of tert-butyl 6-bromo-2-((1-methylcyclopropanecarboxamido)methyl)-1H-indole-1-carboxylate (1.00 g, 2.46 mmol, 1.00 eq), potassium vinyltrifluoroborate (658 mg, 4.91 mmol, 2.00 eq) in i-PrOH (10 mL) were added TEA (1.03 mL, 7.37 mmol, 3.00 eq) and Pd(dppf)Cl₂·CH₂Cl₂ (200 mg, 245 μmol, 0.10 eq) under N₂ atmosphere. The system was degassed and then charged with nitrogen three times. The reaction mixture was heated to 100° C. and stirred for 1 h. The reaction mixture was allowed to cool to room temperature, then was quenched by addition of water (20 mL) and extracted with ethyl acetate (3×20 mL). The combined organic layers were dried over anhydrous Na₂SO₄, filtered, and concentrated under reduced pressure. Purification by column chromatography gave tert-butyl 2-((1-methylcyclopropanecarboxamido)methyl)-6-vinyl-1H-indole-1-carboxylate. LC-MS (ESI) m/z calcd for C₂₁H₂₆N₂O₃: 354.19; found 355.2 [M+H]⁺.

### Step 2: tert-butyl 6-formyl-2-((1-methylcyclopropanecarboxamido)methyl)-1H-indole-1-carboxylate

To a solution of tert-butyl 2-((1-methylcyclopropanecarboxamido)methyl)-6-vinyl-1H-indole-1-carboxylate (800 mg, 2.26 mmol, 1.00 eq) in THF (10 mL) and H₂O (1 mL) were added NaIO₄ (1.93 g, 9.03 mmol, 4.00 eq) and potassium osmate(VI) dihydrate (41.6 mg, 113 μmol, 0.05 eq). The reaction mixture was stirred for 1 h, then was quenched by addition of saturated aqueous Na₂S₂O₃ solution (50 mL) and extracted with ethyl acetate (3×45 mL). The combined organic layers were dried over anhydrous Na₂SO₄, filtered, and concentrated under reduced pressure. Purification by column chromatography gave tert-butyl 6-formyl-2-((1-methylcyclopropanecarboxamido)methyl)-1H-indole-1-carboxylate. LC-MS (ESI) m/z calcd for C₂₀H₂₄N₂O₄: 356.17; found 357.1 [M+H]⁺.

### Step 3: (E)-tert-butyl 6-(2-(isoxazol-3-yl)vinyl)-2-((1-methylcyclopropanecarboxamido)methyl)-1H-indole-1-carboxylate

To a solution of 3-((chlorotriphenylphosphoranyl)methyl)isoxazole (575 mg, 1.52 mmol, 1.50 eq) in THF (4 mL) was added NaH (101 mg, 2.53 mmol, 60% in mineral oil, 2.50 eq) portionwise at 0° C., followed by tert-butyl 6-formyl-2-((1-methylcyclopropanecarboxamido)methyl)-1H-indole-1-carboxylate (360 mg, 1.01 mmol, 1.00 eq). The resulting reaction mixture was stirred at 20° C. for 3 h. The reaction mixture was quenched by addition of water (20 mL) and extracted with ethyl acetate (3×25 mL). The combined organic layers were dried over anhydrous Na₂SO₄, filtered, and concentrated under reduced pressure. Purification by column chromatography gave (E)-tert-butyl 6-(2-(isoxazol-3-yl)vinyl)-2-((1-methylcyclopropanecarboxamido)methyl)-1H-indole-1-carboxylate. LC-MS (ESI) m/z calcd for C₂₄H₂₇N₃O₄: 421.20; found 422.2 [M+H]⁺.

### Step 4: tert-butyl 6-(2-(isoxazol-3-yl)ethyl)-2-((1-methylcyclopropanecarboxamido)methyl)-1H-indole-1-carboxylate

To a solution of (E)-tert-butyl 6-(2-(isoxazol-3-yl)vinyl)-2-((1-methylcyclopropanecarboxamido)methyl)-1H-indole-1-carboxylate (200 mg, 474 μmol, 1.00 eq) in EtOH (3 mL) was added NH₂NH₂—H₂O (730 mg, 14.2 mmol, 30.1 eq) under 02 (15 psi) atmosphere. The reaction mixture was then heated to 80° C. and stirred for 16 h. The reaction mixture was allowed to cool to room temperature, then was quenched by addition of water (15 mL) and extracted with ethyl acetate (3×15 mL). The combined organic layers were dried over anhydrous Na₂SO₄, filtered, and concentrated under reduced pressure to give tert-butyl 6-(2-(isoxazol-3-yl)ethyl)-2-((1-methylcyclopropanecarboxamido)methyl)-1H-indole-1-carboxylate (200 mg), which was used without further purification. LC-MS (ESI) m/z calcd for C₂₄H₂₉N₃O₄: 423.22; found 424.2 [M+H]⁺.

### Step 5: N-((6-(2-(isoxazol-3-yl)ethyl)-1H-indol-2-yl)methyl)-1-methylcyclopropanecarboxamide

To a solution of tert-butyl 6-(2-(isoxazol-3-yl)ethyl)-2-((1-methylcyclopropanecarboxamido)methyl)-1H-indole-1-carboxylate (200 mg, 472 μmol, 1.00 eq) in EtOAc (0.5 mL) was added 4M HCl in EtOAc (1.0 mL, 4.00 mmol, 8.47 eq). The reaction mixture was stirred for 0.5 h, then was quenched by addition of saturation aqueous NaHCO₃ (10 mL) and extracted with ethyl acetate (3×15 mL). The combined organic layers were dried over anhydrous Na₂SO₄, filtered, and concentrated under reduced pressure. Purification by prep-TLC gave N-((6-(2-(isoxazol-3-yl)ethyl)-1H-indol-2-yl)methyl)-1-methylcyclopropanecarboxamide (Compound 75). LC-MS (ESI) m/z calcd for C₁₉H₂₁N₃O₂: 323.16; found 324.2 [M+H]⁺. ¹H NMR (400 MHz, DMSO-d₆) δ 10.65 (s, 1H), 8.75 (s, 1H), 7.98 (t, J=5.6 Hz, 1H), 7.33 (d, J=8.0 Hz, 1H), 7.16 (s, 1H), 6.84 (d, J=7.2 Hz, 1H), 6.46 (d, J=1.6 Hz, 1H), 6.13 (s, 1H), 4.37 (d, J=5.6 Hz, 2H), 3.00-2.94 (m, 4H), 1.29 (s, 3H), 1.00-0.98 (m, 2H), 0.54-0.51 (m, 2H).

The following compounds in Table T-7 were synthesized using procedures similar to Compound 75 using the appropriate starting materials.

TABLE T-7

| Com- | | | Exact | LCMS, |
|---|---|---|---|---|
| pound | | | mass | found |
| No. | Structure | IUPAC name | (g/mol) | [M + H]⁺ |
| 76 | 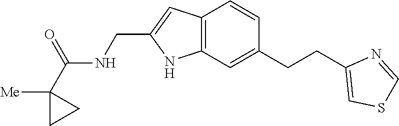 [SMILES: CC1(C(=O)NCc2cc3ccc(CCc4cscn4)cc3[nH]2)CC1] | 1-methyl-N-((6-(2- (thiazol-4-yl)ethyl)-1H- indol-2- yl)methyl)cyclopropane- 1-carboxamide | 339.14 | 340.2 |
| 77 | 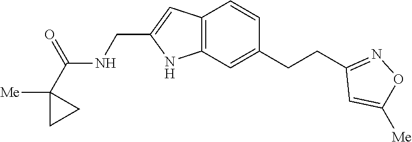 [SMILES: Cc1cc(CCc2ccc3cc(CNC(=O)C4(C)CC4)[nH]c3c2)no1] | 1-methyl-N-((6-(2-(5- methylisoxazol-3- yl)ethyl)-1H-indol-2- yl)methyl)cyclopropane- 1-carboxamide | 337.18 | 338.2 |

### Example 10: (Compound 78)

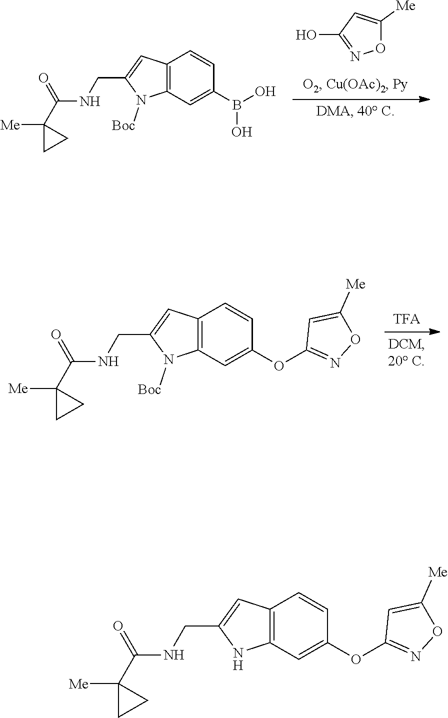 [SMILES: CC(C)(C)OC(=O)n1c(CNC(=O)C2(C)CC2)cc2ccc(B(O)O)cc21 ; Cc1cc(O)no1 ; Cc1cc(Oc2ccc3cc(CNC(=O)C4(C)CC4)n(C(=O)OC(C)(C)C)c3c2)no1 ; Cc1cc(Oc2ccc3cc(CNC(=O)C4(C)CC4)[nH]c3c2)no1]

### Step 1: tert-butyl 2-((1-methylcyclopropanecarboxamido)methyl)-6-((5-methylisoxazol-3-yl)oxy)-1H-indole-1-carboxylate

To a solution of (1-(tert-butoxycarbonyl)-2-((1-methylcyclopropanecarboxamido)methyl)-1H-indol-6-yl)boronic acid (150 mg, 403 μmol, 1.00 eq) in DMA (2 mL) were added 5-methylisoxazol-3-ol (60 mg, 604 μmol, 1.50 eq), pyridine (165 μL, 2.01 mmol, 5.00 eq), Cu(OAc)₂ (183 mg, 1.01 mmol, 2.50 eq) and 4 Å molecular sieves (200 mg). The mixture was heated to 40° C. and stirred for 16 h under 02. The reaction mixture was allowed to cool to room temperature, then was filtered, poured into water (10 mL) and stirred for 2 min. The aqueous phase was extracted with ethyl acetate (2×10 mL). The combined organic phase was washed with brine (2×10 mL), dried over anhydrous Na₂SO₄, filtered, and concentrated under reduced pressure. Purification by prep-HPLC gave tert-butyl 2-((1-methylcyclopropanecarboxamido)methyl)-6-((5-methylisoxazol-3-yl)oxy)-1H-indole-1-carboxylate. LC-MS (ESI) m/z calcd for C₂₃H₂₇N₃O₅: 425.20; found 326.1 [M−Boc+H]⁺.

### Step 2: 1-methyl-N-((6-((5-methylisoxazol-3-yl)oxy)-1H-indol-2-yl)methyl)cyclopropanecarboxamide

To a solution of tert-butyl 2-((1-methylcyclopropanecarboxamido)methyl)-6-((5-methylisoxazol-3-yl)oxy)-1H-indole-1-carboxylate (30 mg, 70.5 μmol, 1.00 eq) in DCM (0.8 mL) was added TFA (0.2 mL), and the mixture was stirred for 0.5 h. The mixture was concentrated in vacuo and purified by prep-HPLC to give 1-methyl-N-((6-((5-methylisoxazol-3-yl)oxy)-1H-indol-2-yl)methyl)cyclopropanecarboxamide (Compound 78). LC-MS (ESI) m/z calcd for C₁₈H₁₉N₃O₃: 325.14; found 326.0 [M+H]⁺. ¹H NMR (400 MHz, DMSO-d₆) δ 10.97 (s, 1H), 8.06 (t, J=6.0 Hz, 1H), 7.63 (s, 1H), 7.49 (d, J=8.4 Hz, 1H), 7.23 (s, 1H), 7.10 (d, J=8.4 Hz, 1H), 6.23 (s, 1H), 4.40 (d, J=6.0 Hz, 2H), 2.13 (s, 3H), 1.30 (s, 3H), 1.01-0.99 (m, 2H), 0.55-0.52 (m, 2H)

### Example 11: (Compound 81)

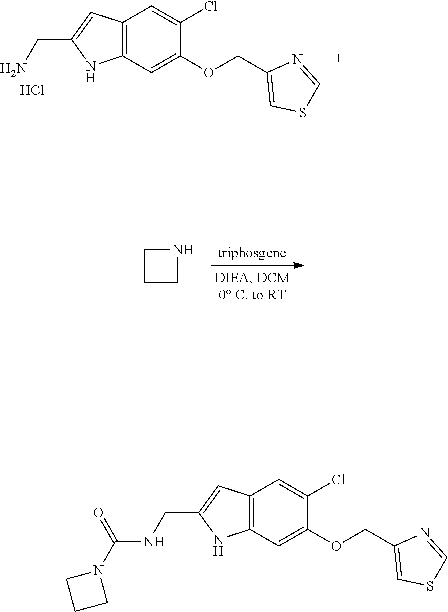

### Step 1: N-((5-chloro-6-(thiazol-4-ylmethoxy)-1H-indol-2-yl)methyl)azetidine-1-carboxamide

A 100 mL three-necked flask equipped with a stir bar was charged with triphosgene (76 mg, 404 μmol, 0.50 eq) and DCM (1.5 mL). The solution was degassed with N₂ three times and cooled to 0° C., then a solution of (5-chloro-6-(thiazol-4-ylmethoxy)-1H-indol-2-yl)methanamine hydrochloride (150 mg, 510 μmol, 1.00 eq), azetidine (68.9 μL, 1.02 mmol, 2.00 eq) and DIEA (355 μL, 2.04 mmol, 4.00 eq) in DCM (1.5 mL) was added dropwise slowly at 0° C. The resulting mixture was allowed to warm to room temperature, then was stirred for 16 h. The reaction mixture was quenched by addition of H₂O (10 mL), then was extracted with EtOAc (3×10 mL). The combined organic layers were washed with brine (5 mL), dried over anhydrous Na₂SO₄, filtered, and concentrated under reduced pressure. Purification by prep-HPLC gave N-((5-chloro-6-(thiazol-4-ylmethoxy)-1H-indol-2-yl)methyl)azetidine-1-carboxamide (Compound 81). LC-MS (ESI) m/z calcd for C₁₇H₁₇ClN₄O₂S: 376.08; found 377.1 [M+H]⁺. ¹H NMR (400 MHz, DMSO-d₆) δ 10.78 (s, 1H), 9.15 (s, 1H), 7.76 (s, 1H), 7.49 (s, 1H), 7.19 (s, 1H), 6.75 (t, J=5.6 Hz, 1H), 6.12 (s, 1H), 5.26 (s, 2H), 4.26 (d, J=6.0 Hz, 2H), 3.82 (t, J=7.6 Hz, 4H), 2.18-2.08 (m, 2H).

The following compounds in Table T-8 were synthesized using procedures similar to Compound 81 using the appropriate starting materials.

TABLE T-8

| Com- | | | Exact | LCMS, |
|---|---|---|---|---|
| pound | | | mass | found |
| No. | Structure | IUPAC name | (g/mol) | [M + H]⁺ |
| 82 | 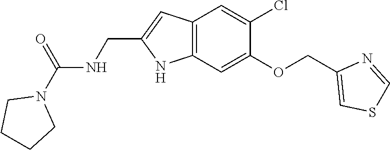 [SMILES: O=C(NCc1cc2cc(Cl)c(OCc3cscn3)cc2[nH]1)N1CCCC1] | N-((5-chloro-6-(thiazol-4- ylmethoxy)-1H-indol-2- yl)methyl)pyrrolidine-1- carboxamide | 390.09 | 391.1 |
| 83 | 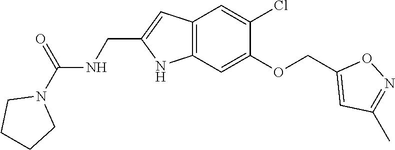 [SMILES: Cc1cc(COc2cc3[nH]c(CNC(=O)N4CCCC4)cc3cc2Cl)on1] | N-((5-chloro-6-((3- methylisoxazol-5- yl)methoxy)-1H-indol-2- yl)methyl)pyrrolidine-1- carboxamide | 388.13 | 389.0 |
| 84 | 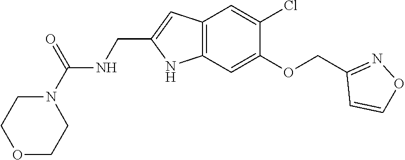 [SMILES: O=C(NCc1cc2cc(Cl)c(OCc3ccon3)cc2[nH]1)N1CCOCC1] | N-((5-chloro-6-(isoxazol-3- ylmethoxy)-1H-indol-2- yl)methyl)morpholine-4- carboxamide | 390.11 | 391.1 |
| 85 | 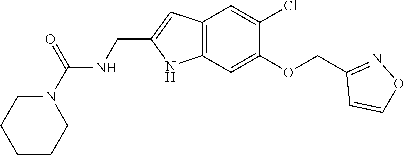 [SMILES: O=C(NCc1cc2cc(Cl)c(OCc3ccon3)cc2[nH]1)N1CCCCC1] | N-((5-chloro-6-(isoxazol-3- ylmethoxy)-1H-indol-2- yl)methyl)piperidine-1- carboxamide | 388.13 | 389.1 |
| 86 | 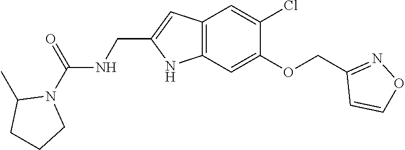 [SMILES: CC1CCCN1C(=O)NCc1cc2cc(Cl)c(OCc3ccon3)cc2[nH]1] | N-((5-chloro-6-(isoxazol-3- ylmethoxy)-1H-indol-2- yl)methyl)-2- methylpyrrolidine-1- carboxamide | 388.13 | 389.1 |
| 87 | 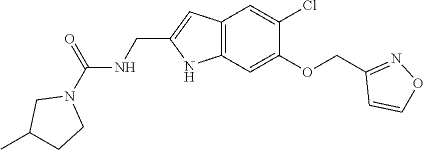 [SMILES: CC1CCN(C(=O)NCc2cc3cc(Cl)c(OCc4ccon4)cc3[nH]2)C1] | N-((5-chloro-6-(isoxazol-3- ylmethoxy)-1H-indol-2- yl)methyl)-3- methylpyrrolidine-1- carboxamide | 388.13 | 389.1 |
| 88 | 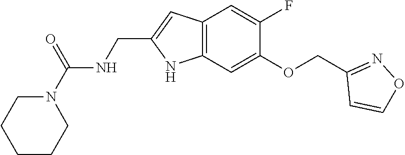 [SMILES: O=C(NCc1cc2cc(F)c(OCc3ccon3)cc2[nH]1)N1CCCCC1] | N-((5-fluoro-6-(isoxazol-3- ylmethoxy)-1H-indol-2- yl)methyl)piperidine-1- carboxamide | 372.16 | 373.0 |
| 89 | 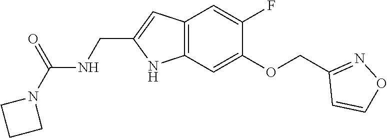 [SMILES: O=C(NCc1cc2cc(F)c(OCc3ccon3)cc2[nH]1)N1CCC1] | N-((5-fluoro-6-(isoxazol-3- ylmethoxy)-1H-indol-2- yl)methyl)azetidine-1- carboxamide | 344.13 | 345.0 |
| 90 | 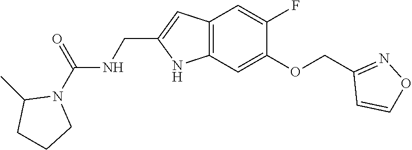 [SMILES: CC1CCCN1C(=O)NCc1cc2cc(F)c(OCc3ccon3)cc2[nH]1] | N-((5-fluoro-6-(isoxazol-3- ylmethoxy)-1H-indol-2- yl)methyl)-2- methylpyrrolidine-1- carboxamide | 372.16 | 373.0 |
| 91 | 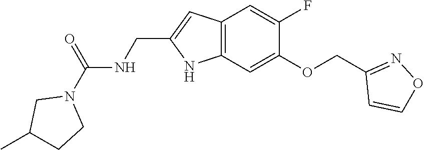 [SMILES: CC1CCN(C(=O)NCc2cc3cc(F)c(OCc4ccon4)cc3[nH]2)C1] | N-((5-fluoro-6-(isoxazol-3- ylmethoxy)-1H-indol-2- yl)methyl)-3- methylpyrrolidine-1- carboxamide | 372.16 | 373.2 |
| 92 | 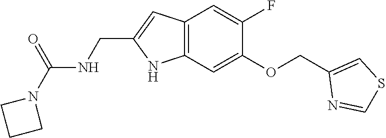 [SMILES: O=C(NCc1cc2cc(F)c(OCc3cscn3)cc2[nH]1)N1CCC1] | N-((5-fluoro-6-(thiazol-4- ylmethoxy)-1H-indol-2- yl)methyl)azetidine-1- carboxamide | 360.11 | 361.1 |
| 93 | 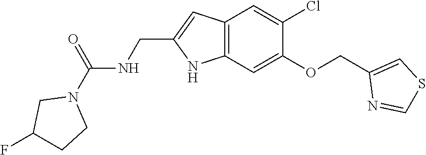 [SMILES: O=C(NCc1cc2cc(Cl)c(OCc3cscn3)cc2[nH]1)N1CCC(F)C1] | N-((5-chloro-6-(thiazol-4- ylmethoxy)-1H-indol-2- yl)methyl)-3- fluoropyrrolidine-1- carboxamide | 408.08 | 409.1 |
| 200 | 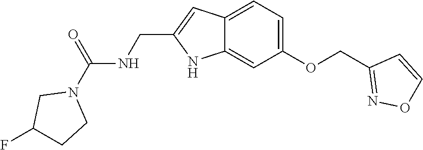 [SMILES: O=C(NCc1cc2ccc(OCc3ccon3)cc2[nH]1)N1CCC(F)C1] | 3-fluoro-N-((6-(isoxazol-3- ylmethoxy)-1H-indol-2- yl)methyl)pyrrolidine-1- carboxamide | 358.14 | 358.8 |
| 357 | 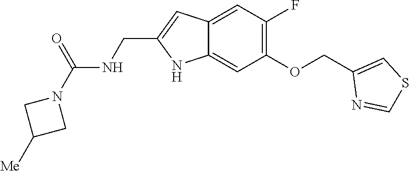 [SMILES: CC1CN(C(=O)NCc2cc3cc(F)c(OCc4cscn4)cc3[nH]2)C1] | N-((5-fluoro-6-(thiazol-4- ylmethoxy)-1H-indol-2- yl)methyl)-3- methylazetidine-1- carboxamide | 374.12 | 375.2 |
| 358 | 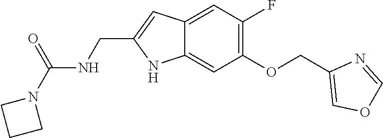 [SMILES: O=C(NCc1cc2cc(F)c(OCc3cocn3)cc2[nH]1)N1CCC1] | N-((5-fluoro-6-(oxazol-4- ylmethoxy)-1H-indol-2- yl)methyl)azetidine-1- carboxamide | 344.13 | 345.1 |
| 359 | 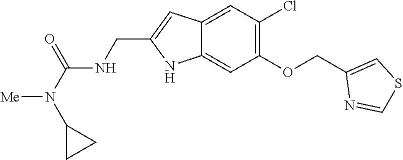 [SMILES: CN(C(=O)NCc1cc2cc(Cl)c(OCc3cscn3)cc2[nH]1)C1CC1] | 3-((5-chloro-6-(thiazol-4- ylmethoxy)-1H-indol-2- yl)methyl)-1-cyclopropyl- 1-methylurea | 390.09 | 391.2 |
| 360 | 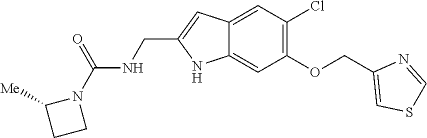 [SMILES: C[C@H]1CCN1C(=O)NCc1cc2cc(Cl)c(OCc3cscn3)cc2[nH]1] | (S)-N-((5-chloro-6-(thiazol- 4-ylmethoxy)-1H-indol-2- yl)methyl)-2- methylazetidine-1- carboxamide | 390.09 | 391.3 |
| 361 | 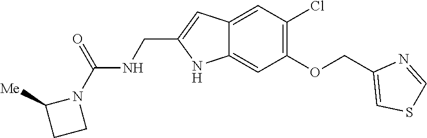 [SMILES: C[C@@H]1CCN1C(=O)NCc1cc2cc(Cl)c(OCc3cscn3)cc2[nH]1] | (R)-N-((5-chloro-6- (thiazol-4-ylmethoxy)-1H- indol-2-yl)methyl)-2- methylazetidine-1- carboxamide | 390.09 | 391.3 |
| 362 | 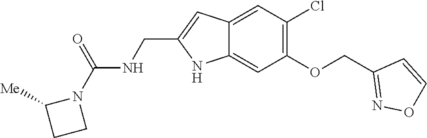 [SMILES: C[C@H]1CCN1C(=O)NCc1cc2cc(Cl)c(OCc3ccon3)cc2[nH]1] | (S)-N-((5-chloro-6- (isoxazol-3-ylmethoxy)- 1H-indol-2-yl)methyl)-2- methylazetidine-1- carboxamide | 374.11 | 375.3 |
| 363 | 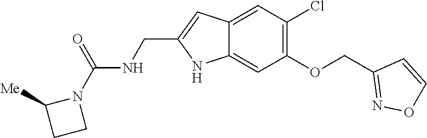 [SMILES: C[C@@H]1CCN1C(=O)NCc1cc2cc(Cl)c(OCc3ccon3)cc2[nH]1] | (R)-N-((5-chloro-6- (isoxazol-3-ylmethoxy)- 1H-indol-2-yl)methyl)-2- methylazetidine-1- carboxamide | 374.11 | 375.3 |
| 364 | 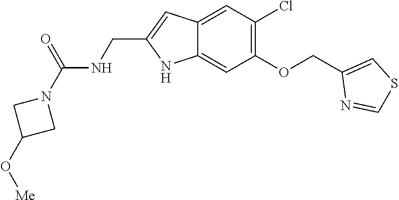 [SMILES: COC1CN(C(=O)NCc2cc3cc(Cl)c(OCc4cscn4)cc3[nH]2)C1] | N-((5-chloro-6-(thiazol-4- ylmethoxy)-1H-indol-2- yl)methyl)-3- methoxyazetidine-1- carboxamide | 406.09 | 407.1 |
| 365 | 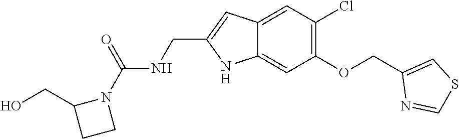 [SMILES: O=C(NCc1cc2cc(Cl)c(OCc3cscn3)cc2[nH]1)N1CCC1CO] | N-((5-chloro-6-(thiazol-4- ylmethoxy)-1H-indol-2- yl)methyl)-2- (hydroxymethyl)azetidine- 1-carboxamide | 406.09 | 407.2 |
| 366 | 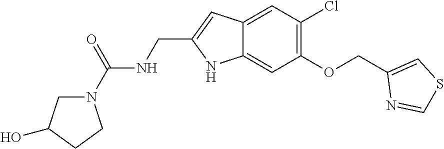 [SMILES: O=C(NCc1cc2cc(Cl)c(OCc3cscn3)cc2[nH]1)N1CCC(O)C1] | N-((5-chloro-6-(thiazol-4- ylmethoxy)-1H-indol-2- yl)methyl)-3- hydroxypyrrolidine-1- carboxamide | 406.09 | 407.2 |
| 367 | 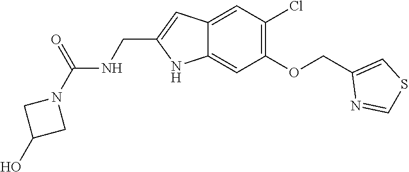 [SMILES: O=C(NCc1cc2cc(Cl)c(OCc3cscn3)cc2[nH]1)N1CC(O)C1] | N-((5-chloro-6-(thiazol-4- ylmethoxy)-1H-indol-2- yl)methyl)-3- hydroxyazetidine-1- carboxamide | 392.07 | 393.1 |
| 368 | 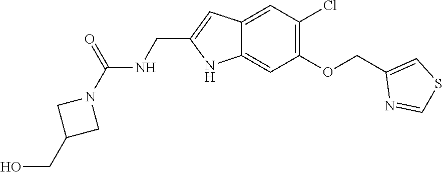 [SMILES: O=C(NCc1cc2cc(Cl)c(OCc3cscn3)cc2[nH]1)N1CC(CO)C1] | N-((5-chloro-6-(thiazol-4- ylmethoxy)-1H-indol-2- yl)methyl)-3- (hydroxymethyl)azetidine- 1-carboxamide | 406.09 | 407.2 |
| 369 | 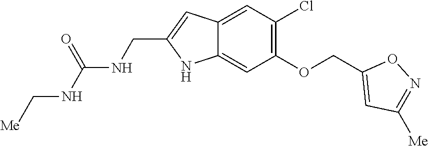 [SMILES: CCNC(=O)NCc1cc2cc(Cl)c(OCc3cc(C)no3)cc2[nH]1] | 1-((5-chloro-6-((3- methylisoxazol-5- yl)methoxy)-1H-indol-2- yl)methyl)-3-ethylurea | 362.11 | 363.1 |
| 370 | 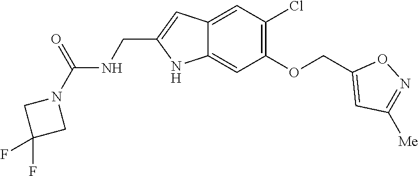 [SMILES: Cc1cc(COc2cc3[nH]c(CNC(=O)N4CC(F)(F)C4)cc3cc2Cl)on1] | N-((5-chloro-6-((3- methylisoxazol-5- yl)methoxy)-1H-indol-2- yl)methyl)-3,3- difluoroazetidine-1- carboxamide | 410.10 | 411.2 |
| 371 | 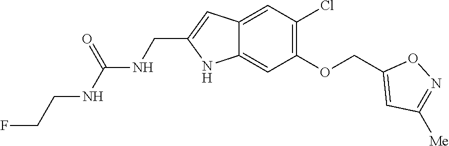 [SMILES: Cc1cc(COc2cc3[nH]c(CNC(=O)NCCF)cc3cc2Cl)on1] | 1-((5-chloro-6-((3- methylisoxazol-5- yl)methoxy)-1H-indol-2- yl)methyl)-3-(2- fluoroethyl)urea | 380.11 | 381.2 |
| 372 | 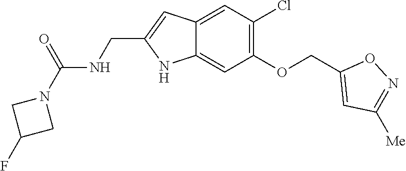 [SMILES: Cc1cc(COc2cc3[nH]c(CNC(=O)N4CC(F)C4)cc3cc2Cl)on1] | N-((5-chloro-6-((3- methylisoxazol-5- yl)methoxy)-1H-indol-2- yl)methyl)-3- fluoroazetidine-1- carboxamide | 392.11 | 393.2 |
| 373 | 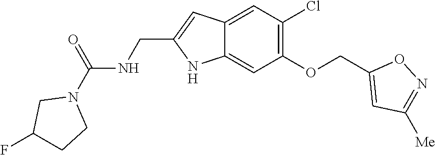 [SMILES: Cc1cc(COc2cc3[nH]c(CNC(=O)N4CCC(F)C4)cc3cc2Cl)on1] | N-((5-chloro-6-((3- methylisoxazol-5- yl)methoxy)-1H-indol-2- yl)methyl)-3- fluoropyrrolidine-1- carboxamide | 406.12 | 407.2 |
| 374 | 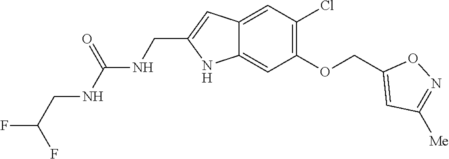 [SMILES: Cc1cc(COc2cc3[nH]c(CNC(=O)NCC(F)F)cc3cc2Cl)on1] | 1-((5-chloro-6-((3- methylisoxazol-5- yl)methoxy)-1H-indol-2- yl)methyl)-3-(2,2- difluoroethyl)urea | 398.10 | 399.2 |
| 375 | 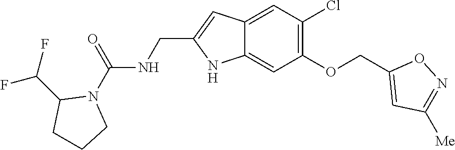 [SMILES: Cc1cc(COc2cc3[nH]c(CNC(=O)N4CCCC4C(F)F)cc3cc2Cl)on1] | N-((5-chloro-6-((3- methylisoxazol-5- yl)methoxy)-1H-indol-2- yl)methyl)-2- (difluoromethyl)pyrrolidine- 1-carboxamide | 438.13 | 439.0 |
| 376 | 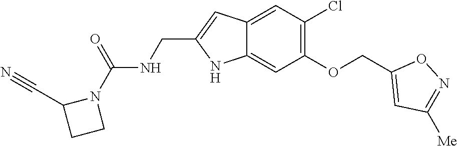 [SMILES: Cc1cc(COc2cc3[nH]c(CNC(=O)N4CCC4C#N)cc3cc2Cl)on1] | N-((5-chloro-6-((3- methylisoxazol-5- yl)methoxy)-1H-indol-2- yl)methyl)-2- cyanoazetidine-1- carboxamide | 399.11 | 400.2 |
| 377 | 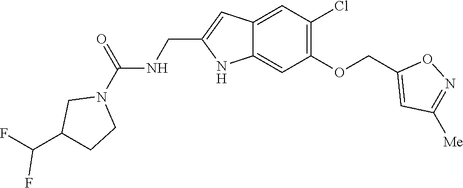 [SMILES: Cc1cc(COc2cc3[nH]c(CNC(=O)N4CCC(C(F)F)C4)cc3cc2Cl)on1] | N-((5-chloro-6-((3- methylisoxazol-5- yl)methoxy)-1H-indol-2- yl)methyl)-3- (difluoromethyl)pyrrolidine- 1-carboxamide | 438.13 | 439.2 |
| 378 | 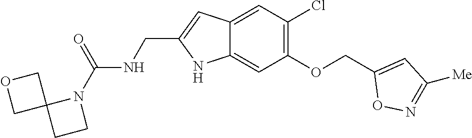 [SMILES: Cc1cc(COc2cc3[nH]c(CNC(=O)N4CCC45COC5)cc3cc2Cl)on1] | N-((5-chloro-6-((3- methylisoxazol-5- yl)methoxy)-1H-indol-2- yl)methyl)-6-oxa-1- azaspiro[3.3]heptane-1- carboxamide | 416.13 | 417.2 |
| 379 | 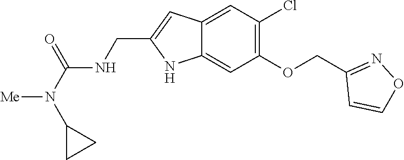 [SMILES: CN(C(=O)NCc1cc2cc(Cl)c(OCc3ccon3)cc2[nH]1)C1CC1] | 3-((5-chloro-6-(isoxazol-3- ylmethoxy)-1H-indol-2- yl)methyl)-1-cyclopropyl- 1-methylurea | 374.11 | 375.2 |
| 380 | 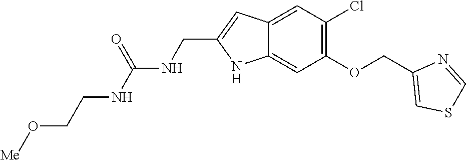 [SMILES: COCCNC(=O)NCc1cc2cc(Cl)c(OCc3cscn3)cc2[nH]1] | 1-((5-chloro-6-(thiazol-4- ylmethoxy)-1H-indol-2- yl)methyl)-3-(2- methoxyethyl)urea | 394.09 | 395.1 |
| 381 | 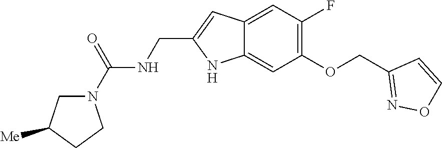 [SMILES: C[C@@H]1CCN(C(=O)NCc2cc3cc(F)c(OCc4ccon4)cc3[nH]2)C1] | (R)-N-((5-fluoro-6- (isoxazol-3-ylmethoxy)- 1H-indol-2-yl)methyl)-3- methylpyrrolidine-1- carboxamide | 372.16 | 373.4 |
| 382 | 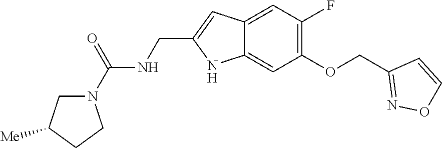 [SMILES: C[C@H]1CCN(C(=O)NCc2cc3cc(F)c(OCc4ccon4)cc3[nH]2)C1] | (S)-N-((5-fluoro-6- (isoxazol-3-ylmethoxy)- 1H-indol-2-yl)methyl)-3- methylpyrrolidine-1- carboxamide | 372.16 | 373.4 |
| 383 | 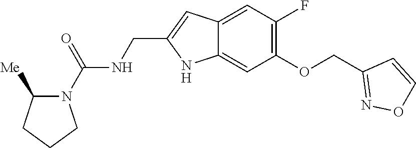 [SMILES: C[C@@H]1CCCN1C(=O)NCc1cc2cc(F)c(OCc3ccon3)cc2[nH]1] | (R)-N-((5-fluoro-6- (isoxazol-3-ylmethoxy)- 1H-indol-2-yl)methyl)-2- methylpyrrolidine-1- carboxamide | 372.16 | 373.4 |
| 384 | 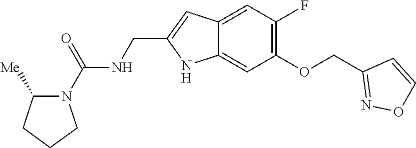 [SMILES: C[C@H]1CCCN1C(=O)NCc1cc2cc(F)c(OCc3ccon3)cc2[nH]1] | (S)-N-((5-fluoro-6- (isoxazol-3-ylmethoxy)- 1H-indol-2-yl)methyl)-2- methylpyrrolidine-1- carboxamide | 372.16 | 373.3 |
| 385 | 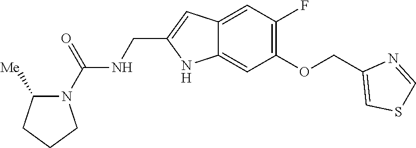 [SMILES: C[C@H]1CCCN1C(=O)NCc1cc2cc(F)c(OCc3cscn3)cc2[nH]1] | (S)-N-((5-fluoro-6-(thiazol- 4-ylmethoxy)-1H-indol-2- yl)methyl)-2- methylpyrrolidine-1- carboxamide | 388.14 | 389.1 |
| 386 | 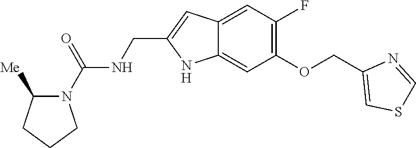 [SMILES: C[C@@H]1CCCN1C(=O)NCc1cc2cc(F)c(OCc3cscn3)cc2[nH]1] | (R)-N-((5-fluoro-6-(thiazol- 4-ylmethoxy)-1H-indol-2- yl)methyl)-2- methylpyrrolidine-1- carboxamide | 388.14 | 389.4 |
| 387 | 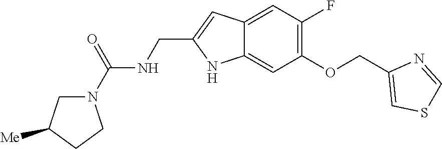 [SMILES: C[C@@H]1CCN(C(=O)NCc2cc3cc(F)c(OCc4cscn4)cc3[nH]2)C1] | (R)-N-((5-fluoro-6-(thiazol- 4-ylmethoxy)-1H-indol-2- yl)methyl)-3- methylpyrrolidine-1- carboxamide | 388.14 | 389.1 |
| 388 | 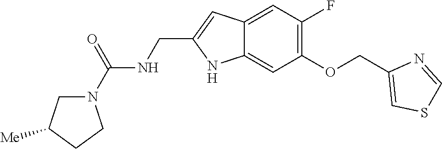 [SMILES: C[C@H]1CCN(C(=O)NCc2cc3cc(F)c(OCc4cscn4)cc3[nH]2)C1] | (S)-N-((5-fluoro-6-(thiazol- 4-ylmethoxy)-1H-indol-2- yl)methyl)-3- methylpyrrolidine-1- carboxamide | 388.14 | 389.1 |
| 389 | 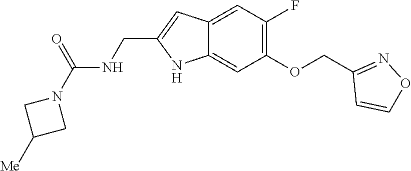 [SMILES: CC1CN(C(=O)NCc2cc3cc(F)c(OCc4ccon4)cc3[nH]2)C1] | N-((5-fluoro-6-(isoxazol-3- ylmethoxy)-1H-indol-2- yl)methyl)-3- methylazetidine-1- carboxamide | 358.14 | 359.2 |
| 390 | 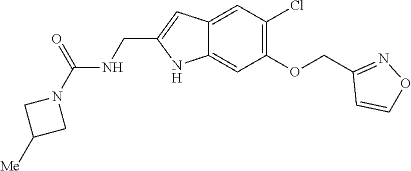 [SMILES: CC1CN(C(=O)NCc2cc3cc(Cl)c(OCc4ccon4)cc3[nH]2)C1] | N-((5-chloro-6-(isoxazol-3- ylmethoxy)-1H-indol-2- yl)methyl)-3- methylazetidine-1- carboxamide | 374.11 | 375.2 |
| 79 | 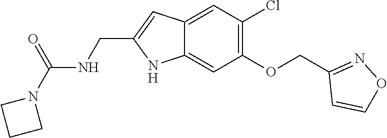 [SMILES: O=C(NCc1cc2cc(Cl)c(OCc3ccon3)cc2[nH]1)N1CCC1] | N-((5-chloro-6-(isoxazol-3- ylmethoxy)-1H-indol-2- yl)methyl)azetidine-1- carboxamide | 360.10 | 361.2 |
| 80 | 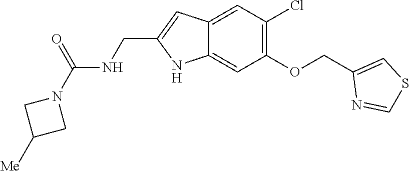 [SMILES: CC1CN(C(=O)NCc2cc3cc(Cl)c(OCc4cscn4)cc3[nH]2)C1] | N-((5-chloro-6-(thiazol-4- ylmethoxy)-1H-indol-2- yl)methyl)-3- methylazetidine-1- carboxamide | 390.09 | 391.2 |

### Example 12: (Compound 94)

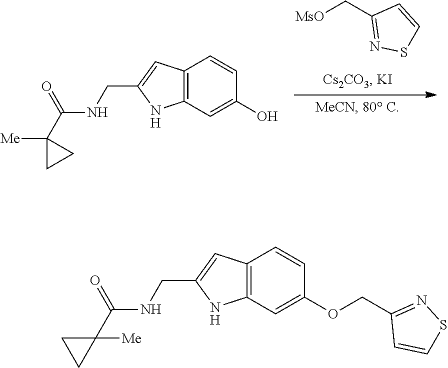 [SMILES: CS(=O)(=O)OCc1ccsn1 ; CC1(C(=O)NCc2cc3ccc(O)cc3[nH]2)CC1 ; CC1(C(=O)NCc2cc3ccc(OCc4ccsn4)cc3[nH]2)CC1]

### Step 1: N-((6-(isothiazol-3-ylmethoxy)-1H-indol-2-yl)methyl)-1-methylcyclopropanecarboxamide

To a mixture of isothiazol-3-ylmethyl methanesulfonate (120 mg, 621 μmol, 1.00 eq) and N-((6-hydroxy-1H-indol-2-yl)methyl)-1-methylcyclopropanecarboxamide (152 mg, 622 μmol, 1.00 eq) in MeCN (5 mL) were added KI (206 mg, 1.24 mmol, 2.00 eq) and Cs₂CO₃ (405 mg, 1.24 mmol, 2.00 eq). The reaction mixture was heated to 80° C. and stirred for 6 h. The mixture was allowed to cool to room temperature, then was quenched by water (30 mL) and extracted with ethyl acetate (3×10 mL). The combined organic layers were washed with brine (10 mL), dried over Na₂SO₄, filtered, and concentrated under reduced pressure. Purification by prep-HPLC gave N-((6-(isothiazol-3-ylmethoxy)-1H-indol-2-yl)methyl)-1-methylcyclopropanecarboxamide (Compound 94). LC-MS (ESI) m/z calcd for C₁₈H₁₉N₃O₂S: 341.12; found 342.1 [M+H]⁺. ¹H NMR (400 MHz, DMSO-d₆) δ 10.59 (s, 1H), 9.07 (d, J=4.8 Hz, 1H), 7.97 (t, J=5.6 Hz, 1H), 7.45 (d, J=4.4 Hz, 1H), 7.31 (d, J=8.4 Hz, 1H), 6.96 (d, J=2.0 Hz, 1H), 6.70-6.67 (m, 1H), 6.11 (s, 1H), 5.22 (s, 2H), 4.34 (d, J=5.6 Hz, 2H), 1.28 (s, 3H), 1.00-0.98 (m, 2H), 0.54-0.51 (m, 2H).

The following compounds in Table T-8.1 were synthesized using procedures similar to Compound 94 using the appropriate starting materials.

TABLE T-8.1

| Com- | | | Exact | LCMS, |
|---|---|---|---|---|
| pound | | | mass | found |
| No. | Structure | IUPAC name | (g/mol) | [M + H]⁺ |
| 391 | 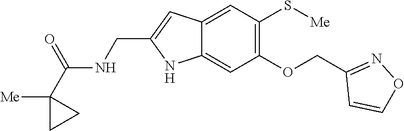 [SMILES: CSc1cc2cc(CNC(=O)C3(C)CC3)[nH]c2cc1OCc1ccon1] | N-((6-(isoxazol-3- ylmethoxy)-5- (methylthio)-1H-indol-2- yl)methyl)-1- methylcyclopropane-1- carboxamide | 371.13 | 372.1 |
| 392 | 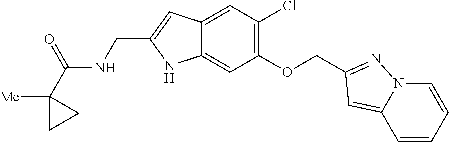 [SMILES: CC1(C(=O)NCc2cc3cc(Cl)c(OCc4cc5ccccn5n4)cc3[nH]2)CC1] | N-((5-chloro-6- (pyrazolo[1,5-a]pyridin-2- ylmethoxy)-1H-indol-2- yl)methyl)-1- methylcyclopropane-1- carboxamide | 408.14 | 409.1 |
| 393 | 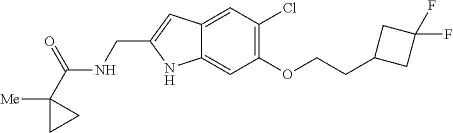 [SMILES: CC1(C(=O)NCc2cc3cc(Cl)c(OCCC4CC(F)(F)C4)cc3[nH]2)CC1] | N-((5-chloro-6-(2-(3,3- difluorocyclobutyl)ethoxy)- 1H-indol-2-yl)methyl)-1- methylcyclopropane-1- carboxamide | 396.14 | 397.1 |
| 394 | 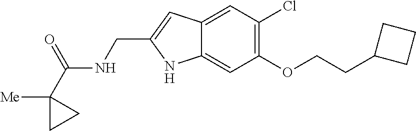 [SMILES: CC1(C(=O)NCc2cc3cc(Cl)c(OCCC4CCC4)cc3[nH]2)CC1] | N-((5-chloro-6-(2- cyclobutylethoxy)-1H- indol-2-yl)methyl)-1- methylcyclopropane-1- carboxamide | 360.16 | 361.2 |
| 395 | 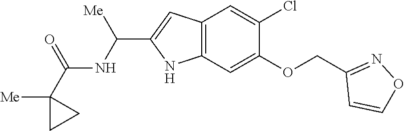 [SMILES: CC(NC(=O)C1(C)CC1)c1cc2cc(Cl)c(OCc3ccon3)cc2[nH]1] | N-(1-(5-chloro-6- (isoxazol-3-ylmethoxy)- 1H-indol-2-yl)ethyl)-1- methylcyclopropane-1- carboxamide | 373.12 | 374.1 |

### Example 13: (Compound 95)

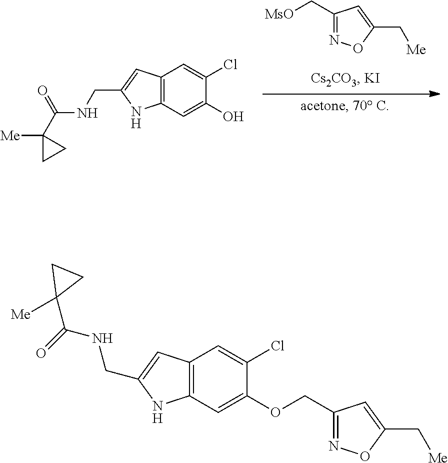 [SMILES: CCc1cc(COS(C)(=O)=O)no1 ; CC1(C(=O)NCc2cc3cc(Cl)c(O)cc3[nH]2)CC1 ; CCc1cc(COc2cc3[nH]c(CNC(=O)C4(C)CC4)cc3cc2Cl)no1]

### Step 1: (N-((5-chloro-6-((5-ethylisoxazol-3-yl)methoxy)-1H-indol-2-yl)methyl)-1-methylcyclopropanecarboxamide

To a mixture of (5-ethylisoxazol-3-yl)methyl methanesulfonate (132 mg, 645 μmol, 1.00 eq) and N-((5-chloro-6-hydroxy-1H-indol-2-yl)methyl)-1-methylcyclopropanecarboxamide (300 mg, 645 μmol, 60% purity, 1.00 eq) in acetone (1 mL) were added Cs₂CO₃ (210 mg, 645 μmol, 1.00 eq) and KI (7.50 mg, 45.2 μmol, 0.07 eq). The mixture was heated to 70° C. and stirred for 4 h. The reaction mixture was allowed to cool to RT, then was poured into water (3 mL) and stirred for 5 min. The aqueous phase was extracted with ethyl acetate (2×5 mL). The combined organic phase was washed with brine (2×5 mL), dried over anhydrous Na₂SO₄, filtered, and concentrated under reduced pressure. Purification by prep-HPLC gave N-((5-chloro-6-((5-ethylisoxazol-3-yl)methoxy)-1H-indol-2-yl)methyl)-1-methylcyclopropanecarboxamide (Compound 95). LC-MS (ESI) m/z calcd for C₂₀H₂₂ClN₃O₃: 387.13; found 388.2 [M+H]⁺. ¹H NMR (400 MHz, DMSO-d₆) δ 10.82 (s, 1H), 8.02 (t, J=5.6 Hz, 1H), 7.50 (s, 1H), 7.18 (s, 1H), 6.36 (s, 1H), 6.12 (s, 1H), 5.19 (s, 2H), 4.35 (d, J=5.6 Hz, 2H), 2.80-2.74 (m, 2H), 1.29 (s, 3H), 1.22 (t, J=7.6 Hz, 3H), 1.01-0.98 (m, 2H), 0.54-0.53 (m, 2H).

### Example 14: (Compound 96)

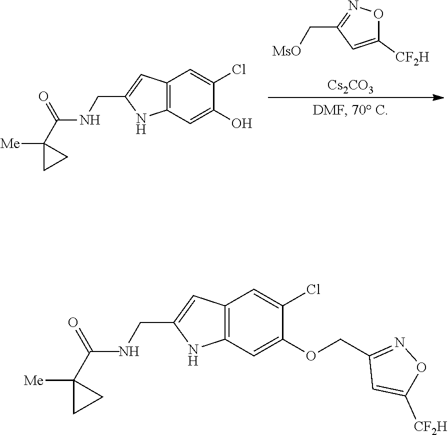

### Step 1: N-((5-chloro-6-((5-(difluoromethyl)isoxazol-3-yl)methoxy)-1H-indol-2-yl)methyl)-1-methylcyclopropane-1-carboxamide

To a solution of (5-(difluoromethyl)isoxazol-3-yl)methyl methanesulfonate (100 mg, 441 μmol, 1.23 eq) and N-((5-chloro-6-hydroxy-1H-indol-2-yl)methyl)-1-methylcyclopropane-1-carboxamide (100 mg, 358 μmol, 1.00 eq) in DMF (2 mL) was added Cs₂CO₃ (233 mg, 717 μmol, 2.00 eq). The resulting mixture was heated to 70° C. and stirred for 1 h. The mixture was then allowed to cool to room temperature, diluted with water (5 mL) and extracted with ethyl acetate (2×5 mL). The combined organic phase was washed with brine (10 mL), dried over anhydrous Na₂SO₄, filtered, and concentrated under reduced pressure. Purification by prep-HPLC gave N-((5-chloro-6-((5-(difluoromethyl)isoxazol-3-yl)methoxy)-1H-indol-2-yl)methyl)-1-methylcyclopropane-1-carboxamide (Compound 96). LC-MS (ESI) m/z calcd for C₁₉H₁₈ClF₂N₃O₃: 409.10; found 409.9 [M+H]⁺. ¹H NMR (400 MHz, DMSO-d₆) δ 10.85 (s, 1H), 8.02 (t, J=5.6 Hz, 1H), 7.51 (s, 1H), 7.50-7.23 (m, 1H), 7.19 (s, 1H), 7.10 (s, 1H), 6.13 (s, 1H), 5.32 (s, 2H), 4.34 (d, J=5.6 Hz, 2H), 1.28 (s, 3H), 1.00-0.98 (m, 2H), 0.54-0.52 (m, 2H).

The following compounds in Table T-9 were synthesized using procedures similar to Compound 96 using the appropriate starting materials.

TABLE T-9

| Com- | | | Exact | LCMS, |
|---|---|---|---|---|
| pound | | | mass | found |
| No. | Structure | IUPAC name | (g/mol) | [M + H]⁺ |
| 97 | 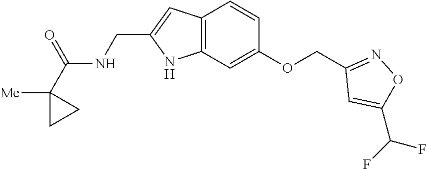 [SMILES: CC1(C(=O)NCc2cc3ccc(OCc4cc(C(F)F)on4)cc3[nH]2)CC1] | N-((6-((5- (difluoromethyl)isoxazol- 3-yl)methoxy)-1H- indol-2-yl)methyl)-1- methylcyclopropane-1- carboxamide | 375.14 | 376.0 |
| 396 | 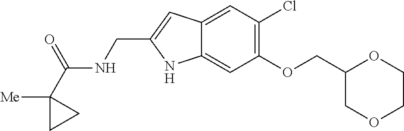 [SMILES: CC1(C(=O)NCc2cc3cc(Cl)c(OCC4COCCO4)cc3[nH]2)CC1] | N-((6-((1,4-dioxan-2- yl)methoxy)-5-chloro- 1H-indol-2-yl)methyl)-1- methylcyclopropane-1- carboxamide | 378.13 | 379.2 |
| 397 | 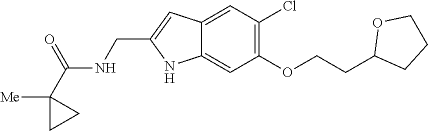 [SMILES: CC1(C(=O)NCc2cc3cc(Cl)c(OCCC4CCCO4)cc3[nH]2)CC1] | N-((5-chloro-6-(2- (tetrahydrofuran-2- yl)ethoxy)-1H-indol-2- yl)methyl)-1- methylcyclopropane-1- carboxamide | 376.16 | 377.3 |
| 398 | 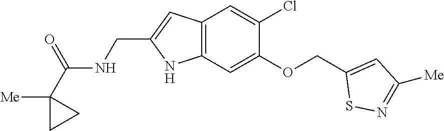 [SMILES: Cc1cc(COc2cc3[nH]c(CNC(=O)C4(C)CC4)cc3cc2Cl)sn1] | N-((5-chloro-6-((3- methylisothiazol-5- yl)methoxy)-1H-indol-2- yl)methyl)-1- methylcyclopropane-1- carboxamide | 389.10 | 390.2 |
| 399 | 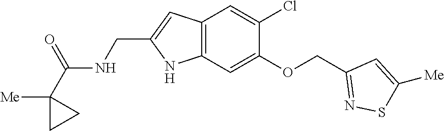 [SMILES: Cc1cc(COc2cc3[nH]c(CNC(=O)C4(C)CC4)cc3cc2Cl)ns1] | N-((5-chloro-6-((5- methylisothiazol-3- yl)methoxy)-1H-indol-2- yl)methyl)-1- methylcyclopropane-1- carboxamide | 389.10 | 390.2 |
| 400 | 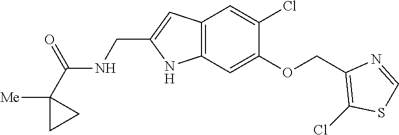 [SMILES: CC1(C(=O)NCc2cc3cc(Cl)c(OCc4ncsc4Cl)cc3[nH]2)CC1] | N-((5-chloro-6-((5- chlorothiazol-4- yl)methoxy)-1H-indol-2- yl)methyl)-1- methylcyclopropane-1- carboxamide | 409.04 | 410.1 |

### Example 15: (Compound 98)

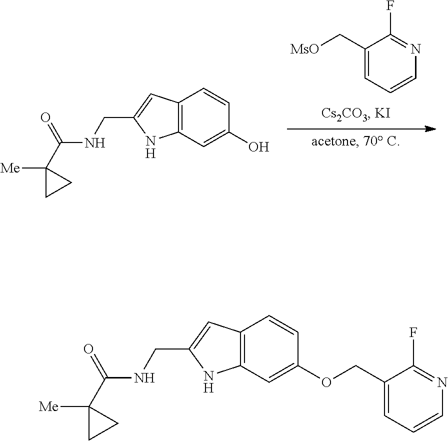 [SMILES: CC1(C(=O)NCc2cc3ccc(O)cc3[nH]2)CC1 ; CS(=O)(=O)OCc1cccnc1F ; CC1(C(=O)NCc2cc3ccc(OCc4cccnc4F)cc3[nH]2)CC1]

### Step 1: N-((6-((2-fluoropyridin-3-yl)methoxy)-1H-indol-2-yl)methyl)-1-methylcyclopropane-1-carboxamide formate

To a solution of (2-fluoropyridin-3-yl)methyl methanesulfonate (150 mg, 614 μmol, 1.00 eq) and N-((6-hydroxy-1H-indol-2-yl)methyl)-1-methylcyclopropane-1-carboxamide (126 mg, 614 μmol, 1.00 eq) in acetone (3 mL) were added Cs₂CO₃ (200 mg, 614 μmol, 1.00 eq) and KI (7.14 mg, 42.9 μmol, 0.07 eq). The mixture was heated to 70° C. and stirred for 4 h. The reaction mixture was allowed to cool to room temperature, then was poured into water (5 mL) and stirred for 3 min. The aqueous phase was extracted with ethyl acetate (3×10 mL). The combined organic phase was washed with brine (4×10 mL) dried over anhydrous Na₂SO₄, filtered, and concentrated under reduced pressure. Purification by prep-HPLC gave N-((6-((2-fluoropyridin-3-yl)methoxy)-1H-indol-2-yl)methyl)-1-methylcyclopropane-1-carboxamide formate (Compound 98). LC-MS (ESI) m/z calcd for C₂₀H₂₀FN₃O₂: 353.15; found 354.1 [M+H]⁺. ¹H NMR (400 MHz, DMSO-d₆) δ 10.58 (s, 1H), 8.20 (d, J=4.8 Hz, 1H), 8.08 (d, J=2.0 Hz, 1H), 7.98 (t, J=5.6 Hz, 1H), 7.40-7.38 (m, 1H), 7.32 (d, J=8.6 Hz, 1H), 6.97 (d, J=2.0 Hz, 1H), 6.70-6.68 (m, 1H), 6.11 (s, 1H), 5.13 (s, 2H), 4.34 (d, J=5.6 Hz, 2H), 1.28 (s, 3H), 1.00-0.97 (m, 2H), 0.53-0.51 (m, 2H).

### Example 16: (Compound 99)

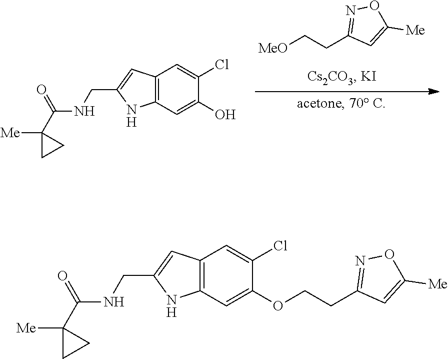 [SMILES: COCCc1cc(C)on1 ; CC1(C(=O)NCc2cc3cc(Cl)c(O)cc3[nH]2)CC1 ; Cc1cc(CCOc2cc3[nH]c(CNC(=O)C4(C)CC4)cc3cc2Cl)no1]

### Step 1: N-((5-chloro-6-(2-(5-methylisoxazol-3-yl)ethoxy)-1H-indol-2-yl)methyl)-1-methylcyclopropane-1-carboxamide

To a solution of 2-(5-methylisoxazol-3-yl)ethyl methanesulfonate (150 mg, 497 μmol, 68% purity, 1.00 eq) and N-((5-chloro-6-hydroxy-1H-indol-2-yl)methyl)-1-methylcyclopropane-1-carboxamide (138 mg, 497 μmol, 1.00 eq) in acetone (2 mL) were added Cs₂CO₃ (161 mg, 497 μmol, 1.00 eq) and KI (5.78 mg, 347 μmol, 0.07 eq). The mixture was heated to 70° C. and stirred for 4 h. The reaction mixture was allowed to cool to room temperature, then was poured into water (5 mL) and stirred for 10 min. The aqueous phase was extracted with ethyl acetate (2×5 mL). The combined organic phase was washed with brine (2×5 mL), dried over anhydrous Na₂SO₄, filtered, and concentrated under reduced pressure. Purification by prep-HPLC gave N-((5-chloro-6-(2-(5-methylisoxazol-3-yl)ethoxy)-1H-indol-2-yl)methyl)-1-methylcyclopropane-1-carboxamide (Compound 99). LC-MS (ESI) m/z calcd for C₂₀H₂₂ClN₃O₂: 387.13; found 388.2 [M+H]⁺. ¹H NMR (400 MHz, DMSO-d₆) δ 10.73 (s, 1H), 8.02 (t, J=5.6 Hz, 1H), 7.47 (s, 1H), 7.07 (s, 1H), 6.28 (s, 1H), 6.11 (s, 1H), 4.35 (d, J=6.0 Hz, 2H), 4.25 (t, J=6.4 Hz, 2H), 3.08 (t, J=6.4 Hz, 2H), 2.37 (s, 3H), 1.28 (s, 3H), 1.02-0.97 (m, 2H), 0.56-0.50 (m, 2H).

The following compounds in Table T-10 were synthesized using procedures similar to Compound 99 using the appropriate starting materials.

TABLE T-10

| Com- | | | Exact | LCMS, |
|---|---|---|---|---|
| pound | | | mass | found |
| No. | Structure | IUPAC name | (g/mol) | [M + H]⁺ |
| 100 | 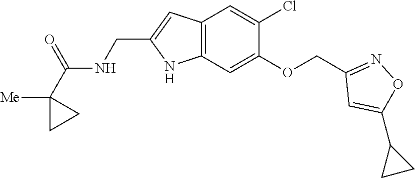 [SMILES: CC1(C(=O)NCc2cc3cc(Cl)c(OCc4cc(C5CC5)on4)cc3[nH]2)CC1] | N-((5-chloro-6-((5- cyclopropylisoxazol-3- yl)methoxy)-1H-indol- 2-yl)methyl)-1- methylcyclopropane-1- carboxamide | 399.13 | 400.1 |
| 101 | 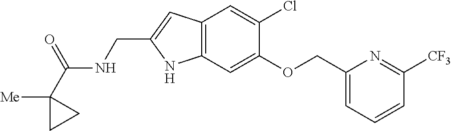 [SMILES: CC1(C(=O)NCc2cc3cc(Cl)c(OCc4cccc(C(F)(F)F)n4)cc3[nH]2)CC1] | N-((5-chloro-6-((6- (trifluoromethyl)pyridin- 2-yl)methoxy)-1H- indol-2-yl)methyl)-1- methylcyclopropane-1- carboxamide | 437.11 | 438.1 |
| 102 | 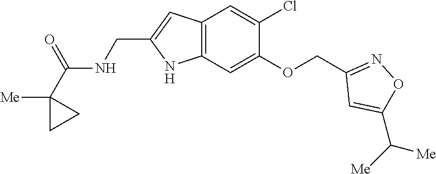 [SMILES: CC(C)c1cc(COc2cc3[nH]c(CNC(=O)C4(C)CC4)cc3cc2Cl)no1] | N-((5-chloro-6-((5- isopropylisoxazol-3- yl)methoxy)-1H-indol- 2-yl)methyl)-1- methylcyclopropane-1- carboxamide | 401.15 | 402.1 |

### Example 17: (Compound 103)

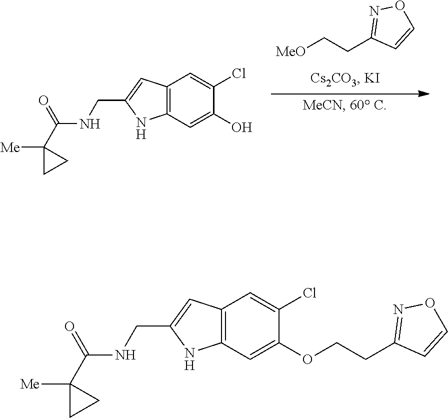 [SMILES: COCCc1ccon1 ; CC1(C(=O)NCc2cc3cc(Cl)c(O)cc3[nH]2)CC1 ; CC1(C(=O)NCc2cc3cc(Cl)c(OCCc4ccon4)cc3[nH]2)CC1]

### Step 1: N-((5-chloro-6-(2-(isoxazol-3-yl)ethoxy)-1H-indol-2-yl)methyl)-1-methylcyclopropane-1-carboxamide

To a mixture of 2-(isoxazol-3-yl)ethyl methanesulfonate (100 mg, 523 μmol, 1.00 eq) and N-((5-chloro-6-hydroxy-1H-indol-2-yl)methyl)-1-methylcyclopropanecarboxamide (150 mg, 538 μmol, 1.00 eq) in MeCN (4 mL) were added Cs₂CO₃ (210 mg, 646 μmol, 1.20 eq) and KI (89 mg, 538 μmol, 1.00 eq). The reaction was heated to 60° C. and stirred for 1 h. The reaction mixture was allowed to cool to room temperature, then was quenched by addition of water (5 mL) and extracted with EtOAc (3×5 mL). The combined organic layers were washed with brine (3×10 mL), dried over Na₂SO₄, filtered, and concentrated under reduced pressure. Purification by prep-HPLC gave N-((5-chloro-6-(2-(isoxazol-3-yl) ethoxy)-1H-indol-2-yl)methyl)-1-methylcyclopropanecarboxamide (Compound 103). LC-MS (ESI) m/z calcd for C₁₉H₂₀ClN₃O₃: 373.12; found 374.0 [M+H]⁺. ¹H NMR (400 MHz, DMSO-d₆) δ 10.73 (s, 1H), 8.84 (s, 1H), 8.02 (t, J=6.0 Hz, 1H), 7.47 (s, 1H), 7.08 (s, 1H), 6.65 (d, J=1.6 Hz, 1H), 6.11 (s, 1H), 4.35 (d, J=5.6 Hz, 2H), 4.29 (t, J=6.4 Hz, 2H), 3.17 (t, J=6.4 Hz, 2H), 1.29 (s, 3H), 1.00-0.98 (m, 2H), 0.54-0.52 (m, 2H).

### Example 18: (Compound 104)

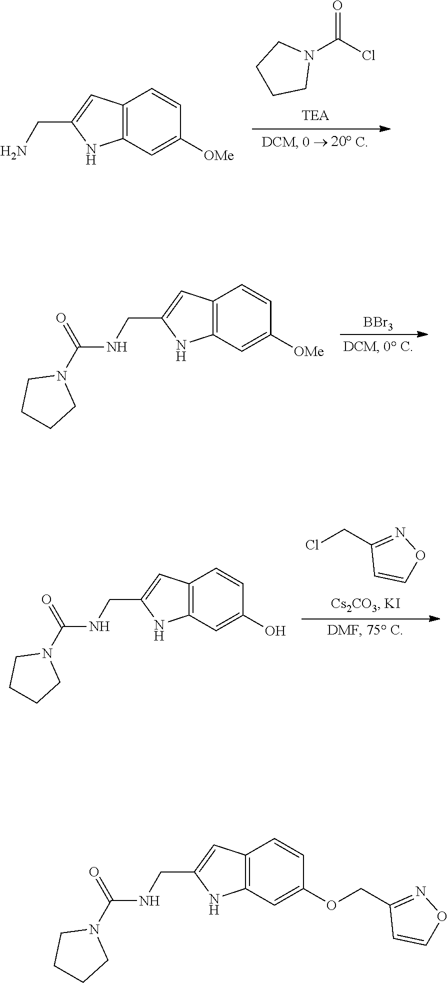

### Step 1: N-((6-methoxy-1H-indol-2-yl)methyl)pyrrolidine-1-carboxamide

To a solution of (6-methoxy-1H-indol-2-yl)methanamine (600 mg, 3.40 mmol, 1.00 eq) and TEA (948 μL, 6.81 mmol, 2.00 eq) in DCM (6 mL) at 0° C. was added pyrrolidine-1-carbonyl chloride (600 μL, 5.11 mmol, 1.50 eq). The reaction was then allowed to warm to 20° C. and stirred for 1 h, then was poured into water (10 mL) and stirred for 5 min. The aqueous phase was extracted with ethyl acetate (2×10 mL). The combined organic phase was washed with brine (2×10 mL), dried over anhydrous Na₂SO₄, filtered, and concentrated under reduced pressure. Purification by column chromatography gave N-((6-methoxy-1H-indol-2-yl)methyl)pyrrolidine-1-carboxamide. LC-MS (ESI) m/z calcd for C₁₅H₁₉N₃O₂: 273.15; found 274.1 [M+H]⁺.

### Step 2: N-((6-hydroxy-1H-indol-2-yl)methyl)pyrrolidine-1-carboxamide

To a solution of N-((6-methoxy-1H-indol-2-yl)methyl)pyrrolidine-1-carboxamide (720 mg, 2.63 mmol, 1.00 eq) in DCM (20 mL) at 0° C. was added BBr₃ (10.5 mmol, 1.02 mL, 4.00 eq) dropwise, and the mixture was stirred at 0° C. for 2 h. The reaction mixture was poured into water (10 mL) and stirred for 5 min. The aqueous phase was extracted with ethyl acetate (2×20 mL). The combined organic phase was washed with brine (2×20 mL), dried over anhydrous Na₂SO₄, filtered, and concentrated under reduced pressure. Purification by column chromatography gave N-((6-hydroxy-1H-indol-2-yl)methyl)pyrrolidine-1-carboxamide. LC-MS (ESI) m/z calcd for C₁₄H₁₇N₃O₂: 259.13; found 260.1 [M+H]⁺.

### Step 3: N-((6-(isoxazol-3-ylmethoxy)-1H-indol-2-yl)methyl)pyrrolidine-1-carboxamide

To a solution of N-((6-hydroxy-1H-indol-2-yl)methyl)pyrrolidine-1-carboxamide (100 mg, 386 μmol, 1.00 eq) and 3-(chloromethyl)isoxazole (55 mg, 462 μmol, 1.20 eq) in DMF (1 mL) were added Cs₂CO₃ (126 mg, 386 μmol, 1.00 eq) and KI (5 mg, 27.0 μmol, 0.07 eq), and the mixture was heated to 75° C. and stirred for 16 h. The reaction mixture was allowed to cool to RT, then was poured into water (5 mL) and stirred for 10 min. The aqueous phase was extracted with ethyl acetate (2×10 mL). The combined organic phase was washed with brine (2×10 mL), dried over anhydrous Na₂SO₄, filtered, and concentrated under reduced pressure. Purification by prep-HPLC gave N-((6-(isoxazol-3-ylmethoxy)-1H-indol-2-yl)methyl)pyrrolidine-1-carboxamide (Compound 104). LC-MS (ESI) m/z calcd for C₁₈H₂₀N₄O₃: 340.15; found 341.1 [M+H]⁺. ¹H NMR (400 MHz, DMSO-d₆) δ 9.45 (s, 1H), 8.40 (s, 1H), 7.42 (d, J=8.0 Hz, 1H), 6.93 (s, 1H), 6.81-6.78 (m, 1H), 6.51 (s, 1H), 6.21 (s, 1H), 5.22 (s, 2H), 4.74-4.71 (m, 1H), 4.42 (d, J=6.0 Hz, 2H), 3.35 (s, 4H), 1.93-1.90 (m, 4H).

The following compounds in Table T-11 were synthesized using procedures similar to Compounds 103 and 104 using the appropriate starting materials.

TABLE T-11

| Com- | | | Exact | LCMS, |
|---|---|---|---|---|
| pound | | | mass | found |
| No. | Structure | IUPAC name | (g/mol) | [M + H]⁺ |
| 105 | 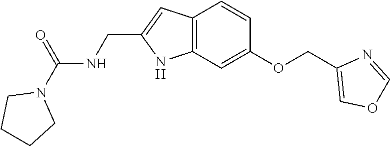 [SMILES: O=C(NCc1cc2ccc(OCc3cocn3)cc2[nH]1)N1CCCC1] | N-((6-(oxazol-4- ylmethoxy)-1H-indol- 2- yl)methyl)pyrrolidine- 1-carboxamide | 340.15 | 341.2 |
| 106 | 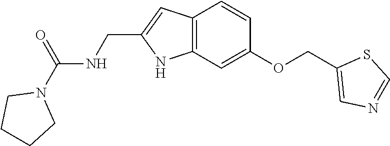 [SMILES: O=C(NCc1cc2ccc(OCc3cncs3)cc2[nH]1)N1CCCC1] | N-((6-(thiazol-5- ylmethoxy)-1H-indol- 2- yl)methyl)pyrrolidine- 1-carboxamide | 356.13 | 357.1 |
| 107 | 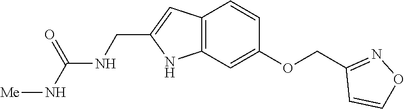 [SMILES: CNC(=O)NCc1cc2ccc(OCc3ccon3)cc2[nH]1] | 1-((6-(isoxazol-3- ylmethoxy)-1H-indol- 2-yl)methyl)-3- methylurea | 300.12 | 301.0 |
| 108 | 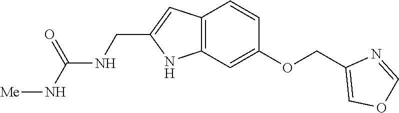 [SMILES: CNC(=O)NCc1cc2ccc(OCc3cocn3)cc2[nH]1] | 1-methyl-3-((6- (oxazol-4- ylmethoxy)-1H-indol- 2-yl)methyl)urea | 300.12 | 300.9 |
| 109 | 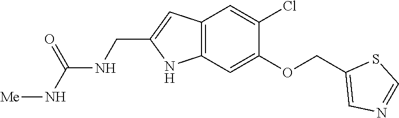 [SMILES: CNC(=O)NCc1cc2cc(Cl)c(OCc3cncs3)cc2[nH]1] | 1-((5-chloro-6- (thiazol-5- ylmethoxy)-1H-indol- 2-yl)methyl)-3- methylurea | 350.06 | 351.0 |
| 110 | 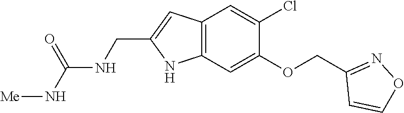 [SMILES: CNC(=O)NCc1cc2cc(Cl)c(OCc3ccon3)cc2[nH]1] | 1-((5-chloro-6- (isoxazol-3- ylmethoxy)-1H-indol- 2-yl)methyl)-3- methylurea | 334.08 | 334.8 |
| 111 | 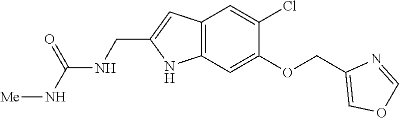 [SMILES: CNC(=O)NCc1cc2cc(Cl)c(OCc3cocn3)cc2[nH]1] | 1-((5-chloro-6- (oxazol-4- ylmethoxy)-1H-indol- 2-yl)methyl)-3- methylurea | 334.08 | 334.8 |
| 112 | 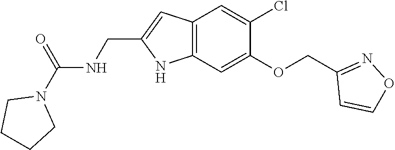 [SMILES: O=C(NCc1cc2cc(Cl)c(OCc3ccon3)cc2[nH]1)N1CCCC1] | N-((5-chloro-6- (isoxazol-3- ylmethoxy)-1H-indol- 2- yl)methyl)pyrrolidine- 1-carboxamide | 374.11 | 375.1 |
| 113 | 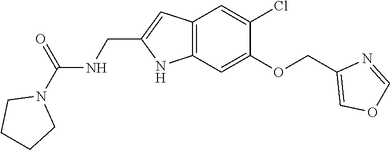 [SMILES: O=C(NCc1cc2cc(Cl)c(OCc3cocn3)cc2[nH]1)N1CCCC1] | N-((5-chloro-6- (oxazol-4- ylmethoxy)-1H-indol- 2- yl)methyl)pyrrolidine- 1-carboxamide | 374.11 | 375.2 |
| 114 | 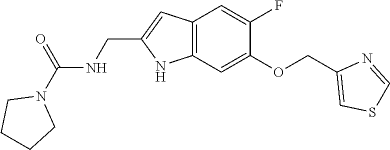 [SMILES: O=C(NCc1cc2cc(F)c(OCc3cscn3)cc2[nH]1)N1CCCC1] | N-((5-fluoro-6- (thiazol-4- ylmethoxy)-1H-indol- 2- yl)methyl)pyrrolidine- 1-carboxamide | 374.12 | 375.0 |
| 115 | 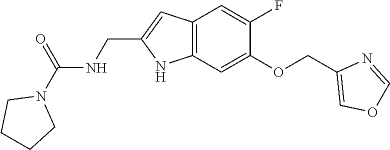 [SMILES: O=C(NCc1cc2cc(F)c(OCc3cocn3)cc2[nH]1)N1CCCC1] | N-((5-fluoro-6- (oxazol-4- ylmethoxy)-1H-indol- 2- yl)methyl)pyrrolidine- 1-carboxamide | 358.14 | 359.0 |
| 116 | 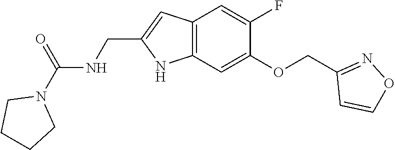 [SMILES: O=C(NCc1cc2cc(F)c(OCc3ccon3)cc2[nH]1)N1CCCC1] | N-((5-fluoro-6- (isoxazol-3- ylmethoxy)-1H-indol- 2- yl)methyl)pyrrolidine- 1-carboxamide | 358.14 | 359.1 |
| 117 | 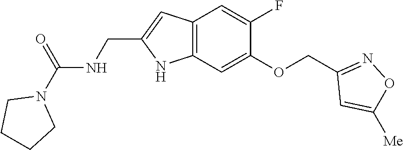 [SMILES: Cc1cc(COc2cc3[nH]c(CNC(=O)N4CCCC4)cc3cc2F)no1] | N-((5-fluoro-6-((5- methylisoxazol-3- yl)methoxy)-1H- indol-2- yl)methyl)pyrrolidine- 1-carboxamide | 372.16 | 373.0 |
| 118 | 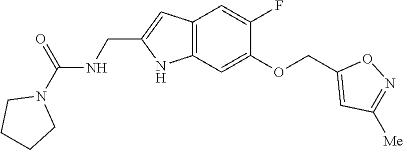 [SMILES: Cc1cc(COc2cc3[nH]c(CNC(=O)N4CCCC4)cc3cc2F)on1] | N-((5-fluoro-6-((3- methylisoxazol-5- yl)methoxy)-1H- indol-2- yl)methyl)pyrrolidine- 1-carboxamide | 372.16 | 373.0 |

### Example 19: (Compound 119)

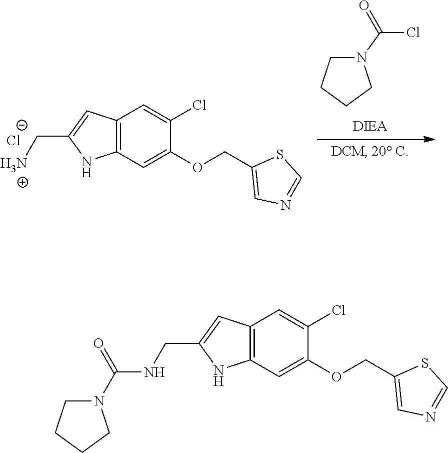 [SMILES: [Cl-] ; [NH3+]Cc1cc2cc(Cl)c(OCc3cncs3)cc2[nH]1 ; O=C(Cl)N1CCCC1 ; O=C(NCc1cc2cc(Cl)c(OCc3cncs3)cc2[nH]1)N1CCCC1]

### Step 1: N-((5-chloro-6-(thiazol-5-ylmethoxy)-1H-indol-2-yl) methyl) pyrrolidine-1-carboxamide

To a solution of pyrrolidine-1-carbonyl chloride (85 μL, 736 μmol, 1.50 eq) and (5-chloro-6-(thiazol-5-ylmethoxy)-1H-indol-2-yl) methanamine hydrogen chloride (180 mg, 491 μmol, 1.00 eq) in DCM (3 mL) was added DIEA (260 μL, 1.47 mmol, 3.00 eq), and the resulting mixture was stirred for 4 h. The reaction mixture was poured into water (10 mL) and stirred for 5 min. The aqueous phase was extracted with ethyl acetate (2×20 mL). The combined organic phase was washed with brine (2×20 mL), dried over anhydrous Na₂SO₄, filtered, and concentrated under reduced pressure. Purification by prep-HPLC to give N-((5-chloro-6-(thiazol-5-ylmethoxy)-1H-indol-2-yl) methyl) pyrrolidine-1-carboxamide (Compound 119). LC-MS (ESI) m/z calcd for C₁₈H₁₉ClN₄O₂S: 390.09; found 391.1 [M+H]⁺. ¹H NMR (400 MHz, DMSO-d₆) δ 9.63 (s, 1H), 8.83 (s, 1H), 7.90 (s, 1H), 7.53 (s, 1H), 6.97 (s, 1H), 6.17 (s, 1H), 5.34 (s, 2H), 4.78 (d, J=4.8 Hz, 1H), 4.41 (d, J=5.2 Hz, 2H), 3.36 (s, 4H), 1.93 (t, J=6.4 Hz, 4H).

The following compounds in Table T-12 were synthesized using procedures similar to Compound 119 using the appropriate starting materials.

TABLE T-12

| Com- | | | Exact | LCMS, |
|---|---|---|---|---|
| pound | | | mass | found |
| No. | Structure | IUPAC name | (g/mol) | [M + H]⁺ |
| 120 | 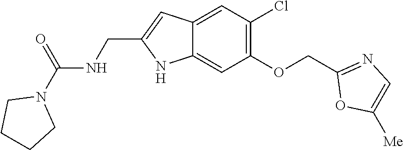 [SMILES: Cc1cnc(COc2cc3[nH]c(CNC(=O)N4CCCC4)cc3cc2Cl)o1] | N-((5-chloro-6-((5- methyloxazol-2- yl)methoxy)-1H- indol-2- yl)methyl)pyrrolidine- 1-carboxamide | 388.13 | 389.0 |
| 121 | 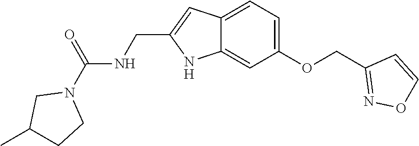 [SMILES: CC1CCN(C(=O)NCc2cc3ccc(OCc4ccon4)cc3[nH]2)C1] | N-((6-(isoxazol-3- ylmethoxy)-1H-indol- 2-yl)methyl)-3- methylpyrrolidine-1- carboxamide | 354.17 | 354.9 |
| 122 | 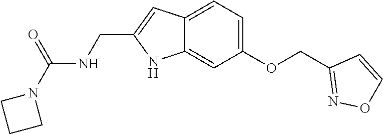 [SMILES: O=C(NCc1cc2ccc(OCc3ccon3)cc2[nH]1)N1CCC1] | N-((6-(isoxazol-3- ylmethoxy)-1H-indol- 2-yl)methyl)azetidine- 1-carboxamide | 326.14 | 326.8 |
| 123 | 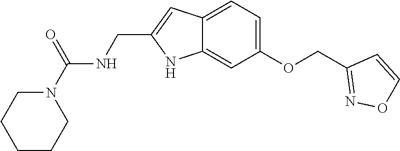 [SMILES: O=C(NCc1cc2ccc(OCc3ccon3)cc2[nH]1)N1CCCCC1] | N-((6-(isoxazol-3- ylmethoxy)-1H-indol- 2- yl)methyl)piperidine- 1-carboxamide | 354.17 | 354.8 |
| 124 | 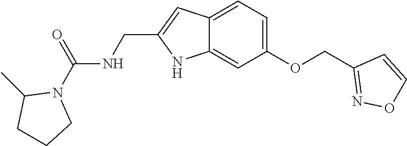 [SMILES: CC1CCCN1C(=O)NCc1cc2ccc(OCc3ccon3)cc2[nH]1] | N-((6-(isoxazol-3- ylmethoxy)-1H-indol- 2-yl)methyl)-2- methylpyrrolidine-1- carboxamide | 354.17 | 354.7 |
| 199 | 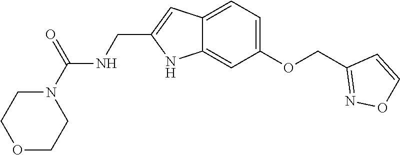 [SMILES: O=C(NCc1cc2ccc(OCc3ccon3)cc2[nH]1)N1CCOCC1] | N-((6-(isoxazol-3- ylmethoxy)-1H-indol- 2- yl)methyl)morpholine- 4-carboxamide | 356.14 | 356.8 |
| 401 | 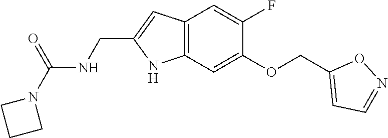 [SMILES: O=C(NCc1cc2cc(F)c(OCc3ccno3)cc2[nH]1)N1CCC1] | N-((5-fluoro-6- (isoxazol-5- ylmethoxy)-1H-indol- 2-yl)methyl)azetidine- 1-carboxamide | 344.13 | 345.2 |
| 402 | 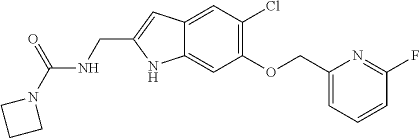 [SMILES: O=C(NCc1cc2cc(Cl)c(OCc3cccc(F)n3)cc2[nH]1)N1CCC1] | N-((5-chloro-6-((6- fluoropyridin-2- yl)methoxy)-1H- indol-2- yl)methyl)azetidine-1- carboxamide | 388.11 | 389.2 |
| 403 | 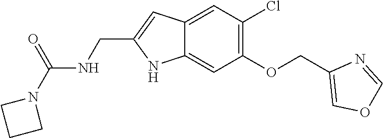 [SMILES: O=C(NCc1cc2cc(Cl)c(OCc3cocn3)cc2[nH]1)N1CCC1] | N-((5-chloro-6- (oxazol-4-ylmethoxy)- 1H-indol-2- yl)methyl)azetidine-1- carboxamide | 360.10 | 361.2 |
| 404 | 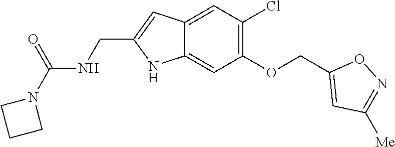 [SMILES: Cc1cc(COc2cc3[nH]c(CNC(=O)N4CCC4)cc3cc2Cl)on1] | N-((5-chloro-6-((3- methylisoxazol-5- yl)methoxy)-1H- indol-2- yl)methyl)azetidine-1- carboxamide | 374.11 | 375.2 |
| 405 | 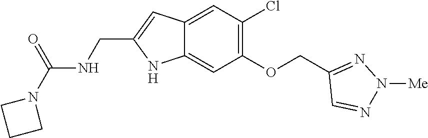 [SMILES: Cn1ncc(COc2cc3[nH]c(CNC(=O)N4CCC4)cc3cc2Cl)n1] | N-((5-chloro-6-((2- methyl-2H-1,2,3- triazol-4-yl)methoxy)- 1H-indol-2- yl)methyl)azetidine-1- carboxamide | 374.13 | 375.1 |
| 406 | 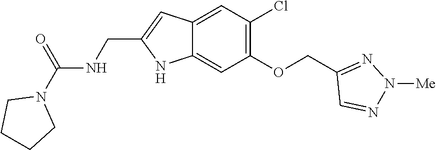 [SMILES: Cn1ncc(COc2cc3[nH]c(CNC(=O)N4CCCC4)cc3cc2Cl)n1] | N-((5-chloro-6-((2- methyl-2H-1,2,3- triazol-4-yl)methoxy)- 1H-indol-2- yl)methyl)pyrrolidine- 1-carboxamide | 388.14 | 389.1 |
| 407 | 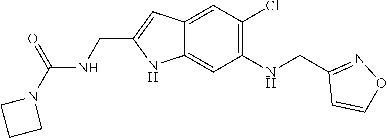 [SMILES: O=C(NCc1cc2cc(Cl)c(NCc3ccon3)cc2[nH]1)N1CCC1] | N-((5-chloro-6- ((isoxazol-3- ylmethyl)amino)-1H- indol-2- yl)methyl)azetidine-1- carboxamide | 359.11 | 360.1 |
| 408 | 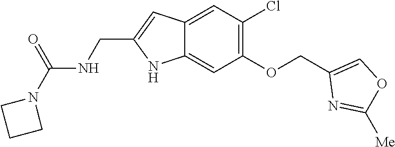 [SMILES: Cc1nc(COc2cc3[nH]c(CNC(=O)N4CCC4)cc3cc2Cl)co1] | N-((5-chloro-6-((2- methyloxazol-4- yl)methoxy)-1H- indol-2- yl)methyl)azetidine-1- carboxamide | 374.11 | 375.2 |
| 409 | 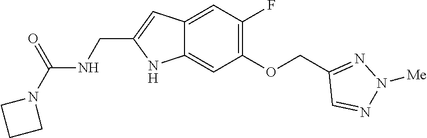 [SMILES: Cn1ncc(COc2cc3[nH]c(CNC(=O)N4CCC4)cc3cc2F)n1] | N-((5-fluoro-6-((2- methyl-2H-1,2,3- triazol-4-yl)methoxy)- 1H-indol-2- yl)methyl)azetidine-1- carboxamide | 358.16 | 359.1 |
| 410 | 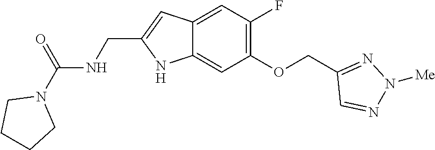 [SMILES: Cn1ncc(COc2cc3[nH]c(CNC(=O)N4CCCC4)cc3cc2F)n1] | N-((5-fluoro-6-((2- methyl-2H-1,2,3- triazol-4-yl)methoxy)- 1H-indol-2- yl)methyl)pyrrolidine- 1-carboxamide | 372.12 | 373.4 |
| 411 | 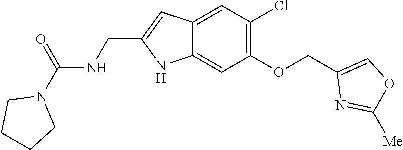 [SMILES: Cc1nc(COc2cc3[nH]c(CNC(=O)N4CCCC4)cc3cc2Cl)co1] | N-((5-chloro-6-((2- methyloxazol-4- yl)methoxy)-1H- indol-2- yl)methyl)pyrrolidine- 1-carboxamide | 388.13 | 389.3 |
| 412 | 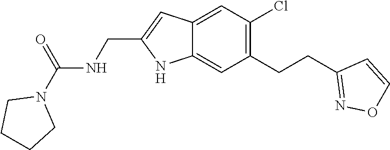 [SMILES: O=C(NCc1cc2cc(Cl)c(CCc3ccon3)cc2[nH]1)N1CCCC1] | N-((5-chloro-6-(2- (isoxazol-3-yl)ethyl)- 1H-indol-2- yl)methyl)pyrrolidine- 1-carboxamide | 372.14 | 373.1 |
| 413 | 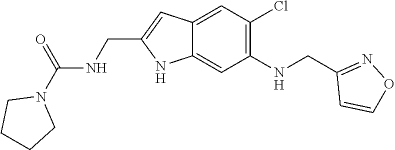 [SMILES: O=C(NCc1cc2cc(Cl)c(NCc3ccon3)cc2[nH]1)N1CCCC1] | N-((5-chloro-6- ((isoxazol-3- ylmethyl)amino)-1H- indol-2- yl)methyl)pyrrolidine- 1-carboxamide | 373.13 | 374.1 |
| 414 | 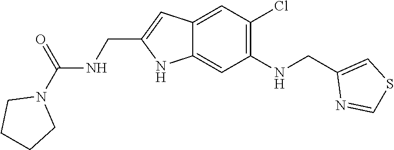 [SMILES: O=C(NCc1cc2cc(Cl)c(NCc3cscn3)cc2[nH]1)N1CCCC1] | N-((5-chloro-6- ((thiazol-4- ylmethyl)amino)-1H- indol-2- yl)methyl)pyrrolidine- 1-carboxamide | 389.11 | 390.1 |
| 415 | 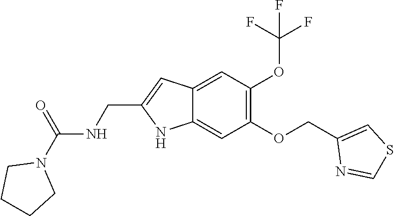 [SMILES: O=C(NCc1cc2cc(OC(F)(F)F)c(OCc3cscn3)cc2[nH]1)N1CCCC1] | N-((6-(thiazol-4- ylmethoxy)-5- (trifluoromethoxy)- 1H-indol-2- yl)methyl)pyrrolidine- 1-carboxamide | 440.11 | 441.1 |
| 416 | 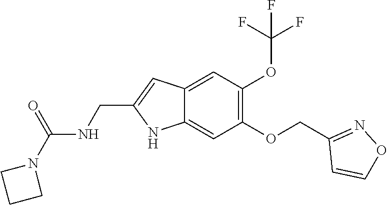 [SMILES: O=C(NCc1cc2cc(OC(F)(F)F)c(OCc3ccon3)cc2[nH]1)N1CCC1] | N-((6-(isoxazol-3- ylmethoxy)-5- (trifluoromethoxy)- 1H-indol-2- yl)methyl)azetidine-1- carboxamide | 410.12 | 411.1 |
| 417 | 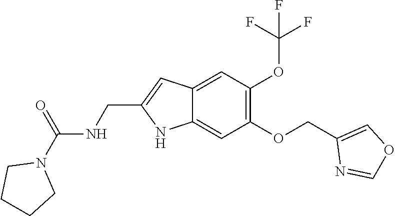 [SMILES: O=C(NCc1cc2cc(OC(F)(F)F)c(OCc3cocn3)cc2[nH]1)N1CCCC1] | N-((6-(oxazol-4- ylmethoxy)-5- (trifluoromethoxy)- 1H-indol-2- yl)methyl)pyrrolidine- 1-carboxamide | 424.14 | 425.3 |
| 418 | 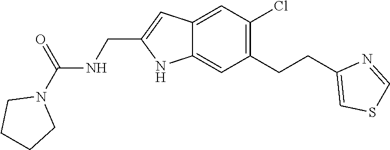 [SMILES: O=C(NCc1cc2cc(Cl)c(CCc3cscn3)cc2[nH]1)N1CCCC1] | N-((5-chloro-6-(2- (thiazol-4-yl)ethyl)- 1H-indol-2- yl)methyl)pyrrolidine- 1-carboxamide | 388.11 | 389.3 |
| 419 | 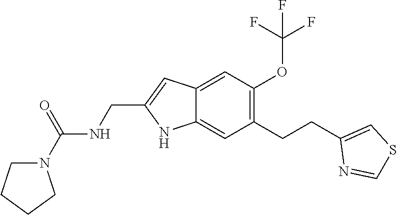 [SMILES: O=C(NCc1cc2cc(OC(F)(F)F)c(CCc3cscn3)cc2[nH]1)N1CCCC1] | N-((6-(2-(thiazol-4- yl)ethyl)-5- (trifluoromethoxy)- 1H-indol-2- yl)methyl)pyrrolidine- 1-carboxamide | 438.13 | 439.1 |
| 420 | 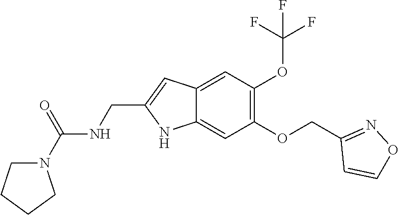 [SMILES: O=C(NCc1cc2cc(OC(F)(F)F)c(OCc3ccon3)cc2[nH]1)N1CCCC1] | N-((6-(isoxazol-3- ylmethoxy)-5- (trifluoromethoxy)- 1H-indol-2- yl)methyl)pyrrolidine- 1-carboxamide | 424.14 | 425.1 |
| 421 | 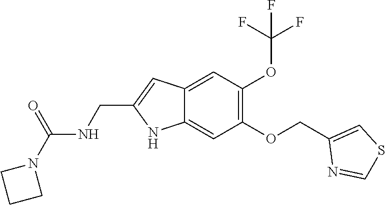 [SMILES: O=C(NCc1cc2cc(OC(F)(F)F)c(OCc3cscn3)cc2[nH]1)N1CCC1] | N-((6-(thiazol-4- ylmethoxy)-5- (trifluoromethoxy)- 1H-indol-2- yl)methyl)azetidine-1- carboxamide | 426.10 | 427.1 |
| 422 | 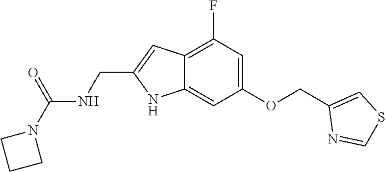 [SMILES: O=C(NCc1cc2c(F)cc(OCc3cscn3)cc2[nH]1)N1CCC1] | N-((4-fluoro-6- (thiazol-4-ylmethoxy)- 1H-indol-2- yl)methyl)azetidine-1- carboxamide | 360.11 | 361.1 |
| 423 | 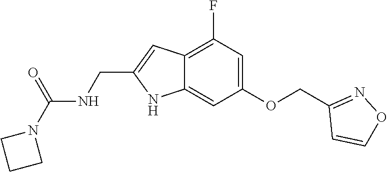 [SMILES: O=C(NCc1cc2c(F)cc(OCc3ccon3)cc2[nH]1)N1CCC1] | N-((4-fluoro-6- (isoxazol-3- ylmethoxy)-1H-indol- 2-yl)methyl)azetidine- 1-carboxamide | 344.13 | 345.1 |
| 424 | 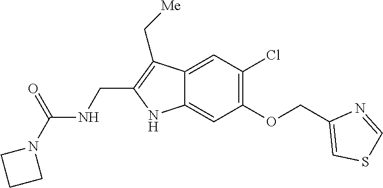 [SMILES: CCc1c(CNC(=O)N2CCC2)[nH]c2cc(OCc3cscn3)c(Cl)cc12] | N-((5-chloro-3-ethyl- 6-(thiazol-4- ylmethoxy)-1H-indol- 2-yl)methyl)azetidine- 1-carboxamide | 404.11 | 403.1 [M − H]⁻ |
| 425 | 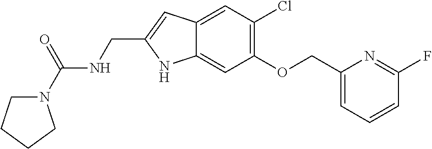 [SMILES: O=C(NCc1cc2cc(Cl)c(OCc3cccc(F)n3)cc2[nH]1)N1CCCC1] | N-((5-chloro-6-((6- fluoropyridin-2- yl)methoxy)-1H- indol-2- yl)methyl)pyrrolidine- 1-carboxamide | 402.13 | 403.1 |
| 426 | 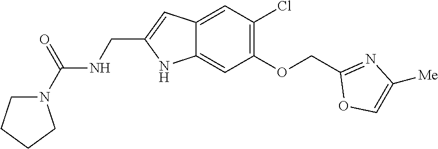 [SMILES: Cc1coc(COc2cc3[nH]c(CNC(=O)N4CCCC4)cc3cc2Cl)n1] | N-((5-chloro-6-((4- methyloxazol-2- yl)methoxy)-1H- indol-2- yl)methyl)pyrrolidine- 1-carboxamide | 388.13 | 389.2 |
| 427 | 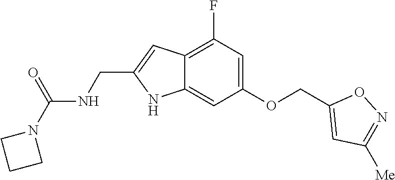 [SMILES: Cc1cc(COc2cc(F)c3cc(CNC(=O)N4CCC4)[nH]c3c2)on1] | N-((4-fluoro-6-((3- methylisoxazol-5- yl)methoxy)-1H- indol-2- yl)methyl)azetidine-1- carboxamide | 358.14 | 359.2 |

### Example 20: (Compound 125)

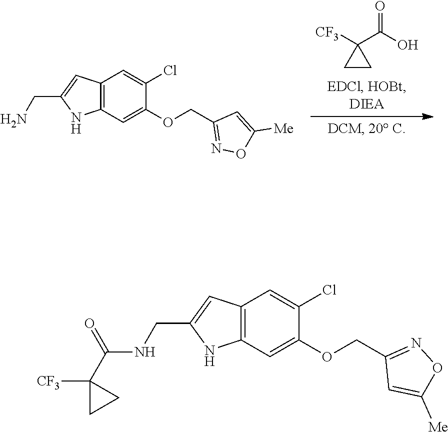 [SMILES: O=C(O)C1(C(F)(F)F)CC1 ; Cc1cc(COc2cc3[nH]c(CN)cc3cc2Cl)no1 ; Cc1cc(COc2cc3[nH]c(CNC(=O)C4(C(F)(F)F)CC4)cc3cc2Cl)no1]

### Step 1: N-((5-chloro-6-((5-methylisoxazol-3-yl)methoxy)-1H-indol-2-yl)methyl)-1-(trifluoromethyl)cyclopropane-1-carboxamide

To a solution of 1-(trifluoromethyl)cyclopropanecarboxylic acid (47.0 mg, 304 μmol, 1.00 eq) in DCM (1 mL) were added HOBt (45.0 mg, 335 μmol, 1.10 eq) and EDCI (64.0 mg, 335 μmol, 1.10 eq) under N₂. The mixture was stirred for 0.5 h, then (5-chloro-6-((5-methylisoxazol-3-yl)methoxy)-1H-indol-2-yl)methanamine (100 mg, 305 μmol, 1.00 eq) and DIEA (106 μL, 609 μmol, 2.00 eq) were added, and the resulting mixture and was stirred for 1.5 h. The reaction was quenched with addition of water (5 mL), then extracted with ethyl acetate (3×10 mL). The combined organic phases were washed with brine, dried over anhydrous Na₂SO₄, filtered, and concentrated under reduced pressure. Purification by prep-HPLC gave N-((5-chloro-6-((5-methylisoxazol-3-yl)methoxy)-1H-indol-2-yl)methyl)-1-(trifluoromethyl)cyclopropane-1-carboxamide (Compound 125). LC-MS (ESI) m/z calcd for C₁₉H₁₇ClF₃N₃O₃: 427.09; found 428.1 [M+H]⁺. ¹H NMR (400 MHz, DMSO-d₆) δ 10.90 (s, 1H), 8.27 (t, J=5.6 Hz, 1H), 7.50 (s, 1H), 7.15 (s, 1H), 6.33 (s, 1H), 6.12 (s, 1H), 5.20 (s, 2H), 4.36 (d, J=5.6 Hz, 2H), 2.41 (s, 3H), 1.37-1.35 (m, 2H), 1.25-1.22 (m, 2H).

The following compounds in Table T-12.1 were synthesized using procedures similar to Compound 125 using the appropriate starting materials.

TABLE T-12.1

| Com- | | | Exact | LCMS, |
|---|---|---|---|---|
| pound | | | mass | found |
| No. | Structure | IUPAC name | (g/mol) | [M + H]⁺ |
| 205 | 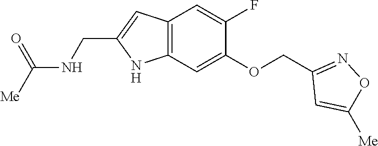 [SMILES: CC(=O)NCc1cc2cc(F)c(OCc3cc(C)on3)cc2[nH]1] | N-((5-fluoro-6-((5- methylisoxazol-3- yl)methoxy)-1H- indol-2- yl)methyl)acetamide | 317.12 | 318.2 |
| 206 | 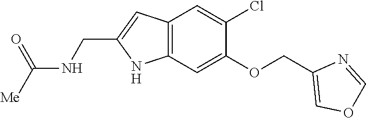 [SMILES: CC(=O)NCc1cc2cc(Cl)c(OCc3cocn3)cc2[nH]1] | N-((5-chloro-6- (oxazol-4- ylmethoxy)-1H-indol- 2- yl)methyl)acetamide | 319.07 | 320.1 |
| 207 | 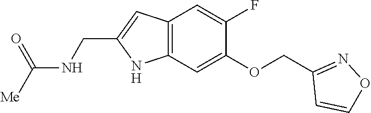 [SMILES: CC(=O)NCc1cc2cc(F)c(OCc3ccon3)cc2[nH]1] | N-((5-fluoro-6- (isoxazol-3- ylmethoxy)-1H-indol- 2- yl)methyl)acetamide | 303.10 | 304.2 |
| 208 | 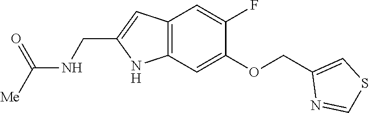 [SMILES: CC(=O)NCc1cc2cc(F)c(OCc3cscn3)cc2[nH]1] | N-((5-fluoro-6- (thiazol-4- ylmethoxy)-1H-indol- 2- yl)methyl)acetamide | 319.08 | 320.1 |

### Example 21: (Compound 126)

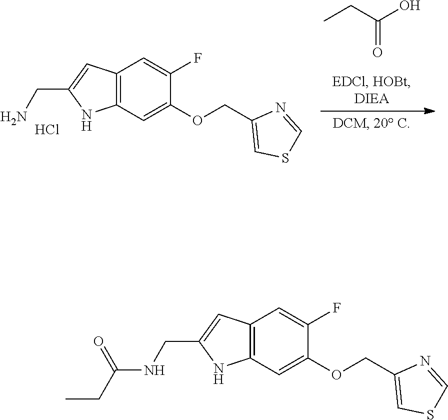

### Step 1: N-((5-fluoro-6-(thiazol-4-ylmethoxy)-1H-indol-2-yl)methyl)propionamide

To a solution of propionic acid (23.6 mg, 318 μmol, 1.00 eq) in DCM (1.5 mL) were added HOBt (47.3 mg, 350 μmol, 1.10 eq) and EDCI (67.2 mg, 350 μmol, 1.10 eq). The mixture was stirred for 0.5 h, then DIEA (111 μL, 637 μmol, 2.00 eq) and (5-fluoro-6-(thiazol-4-ylmethoxy)-1H-indol-2-yl)methanamine hydrochloride (100 mg, 318 μmol, 1.00 eq) were added, and the resulting mixture was stirred for 1 h. The reaction mixture was then poured into H₂O (3 mL). The aqueous phase was extracted with ethyl acetate (3×3 mL), and the combined organic phase was washed with brine (2×3 mL), dried with anhydrous Na₂SO₄, filtered, and concentrated under reduced pressure. Purification by prep-HPLC gave N-((5-fluoro-6-(thiazol-4-ylmethoxy)-1H-indol-2-yl)methyl)propionamide (Compound 126). LC-MS (ESI) m/z calcd for C₁₆H₁₆FN₃O₂S: 333.09; found 334.0 [M+H]⁺. ¹H NMR (400 MHz, DMSO-d₆) δ 10.80 (s, 1H), 9.14 (s, 1H), 8.21 (t, J=5.2 Hz, 1H), 7.77 (s, 1H), 7.24 (d, J=12.0 Hz, 1H), 7.14 (d, J=7.2 Hz, 1H), 6.16 (d, J=1.2 Hz, 1H), 5.25 (s, 2H), 4.33 (d, J=5.6 Hz, 2H), 2.14 (q, J=7.6 Hz, 2H), 1.03 (t, J=7.6 Hz, 3H).

The following compounds in Table T-13 were synthesized using procedures similar to Compounds 125 and 126 using the appropriate starting materials.

TABLE T-13

| Com- | | | Exact | LCMS, |
|---|---|---|---|---|
| pound | | | mass | found |
| No. | Structure | IUPAC name | (g/mol) | [M + H]⁺ |
| 127 | 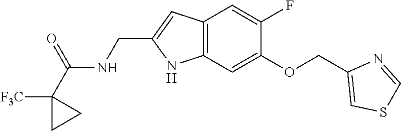 [SMILES: O=C(NCc1cc2cc(F)c(OCc3cscn3)cc2[nH]1)C1(C(F)(F)F)CC1] | N-((5-fluoro-6-(thiazol- 4-ylmethoxy)-1H-indol- 2-yl)methyl)-1- (trifluoromethyl) cyclopropane-1- carboxamide | 413.08 | 413.9 |
| 128 | 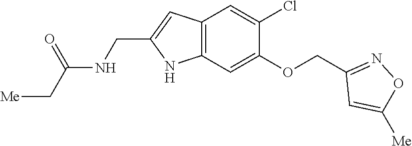 [SMILES: CCC(=O)NCc1cc2cc(Cl)c(OCc3cc(C)on3)cc2[nH]1] | N-((5-chloro-6-((5- methylisoxazol-3- yl)methoxy)-1H-indol- 2- yl)methyl)propionamide | 347.10 | 348.1 |
| 129 | 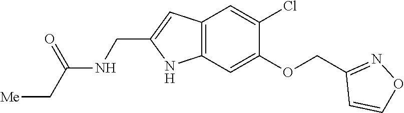 [SMILES: CCC(=O)NCc1cc2cc(Cl)c(OCc3ccon3)cc2[nH]1] | N-((5-chloro-6- (isoxazol-3-ylmethoxy)- 1H-indol-2- yl)methyl)propionamide | 333.09 | 334.1 |
| 130 | 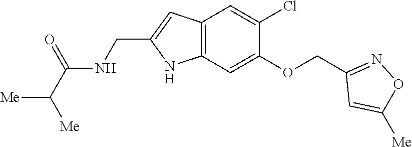 [SMILES: Cc1cc(COc2cc3[nH]c(CNC(=O)C(C)C)cc3cc2Cl)no1] | N-((5-chloro-6-((5- methylisoxazol-3- yl)methoxy)-1H-indol- 2- yl)methyl)isobutyramide | 361.12 | 334.1 |
| 131 | 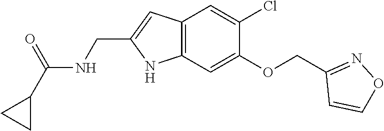 [SMILES: O=C(NCc1cc2cc(Cl)c(OCc3ccon3)cc2[nH]1)C1CC1] | N-((5-chloro-6- (isoxazol-3-ylmethoxy)- 1H-indol-2- yl)methyl)cyclopropane carboxamide | 345.09 | 346.1 |
| 132 | 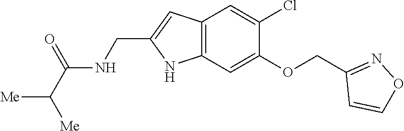 [SMILES: CC(C)C(=O)NCc1cc2cc(Cl)c(OCc3ccon3)cc2[nH]1] | N-((5-chloro-6- (isoxazol-3-ylmethoxy)- 1H-indol-2- yl)methyl)isobutyramide | 347.10 | 348.2 |
| 133 | 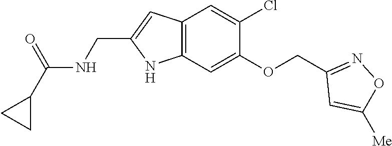 [SMILES: Cc1cc(COc2cc3[nH]c(CNC(=O)C4CC4)cc3cc2Cl)no1] | N-((5-chloro-6-((5- methylisoxazol-3- yl)methoxy)-1H-indol- 2- yl)methyl)cyclopropane carboxamide | 359.10 | 360.1 |
| 134 | 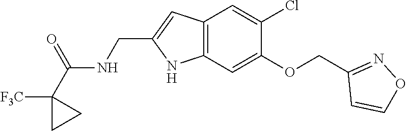 [SMILES: O=C(NCc1cc2cc(Cl)c(OCc3ccon3)cc2[nH]1)C1(C(F)(F)F)CC1] | N-((5-chloro-6- (isoxazol-3-ylmethoxy)- 1H-indol-2-yl)methyl)- 1-(trifluoromethyl) cyclopropane- 1-carboxamide | 413.08 | 414.0 |
| 135 | 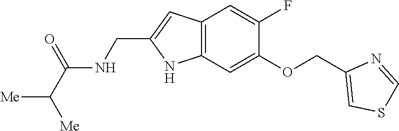 [SMILES: CC(C)C(=O)NCc1cc2cc(F)c(OCc3cscn3)cc2[nH]1] | N-((5-fluoro-6-(thiazol- 4-ylmethoxy)-1H-indol- 2- yl)methyl)isobutyramide | 347.11 | 348.0 |
| 136 | 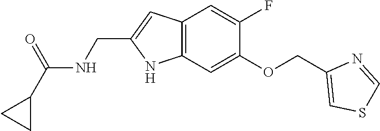 [SMILES: O=C(NCc1cc2cc(F)c(OCc3cscn3)cc2[nH]1)C1CC1] | N-((5-fluoro-6-(thiazol- 4-ylmethoxy)-1H-indol- 2- yl)methyl)cyclopropane carboxamide | 345.09 | 346.1 |
| 137 | 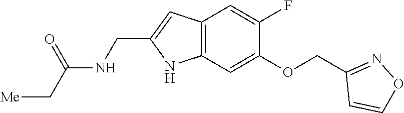 [SMILES: CCC(=O)NCc1cc2cc(F)c(OCc3ccon3)cc2[nH]1] | N-((5-fluoro-6- (isoxazol-3-ylmethoxy)- 1H-indol-2- yl)methyl)propionamide | 317.12 | 318.2 |
| 138 | 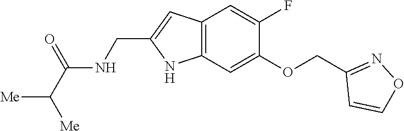 [SMILES: CC(C)C(=O)NCc1cc2cc(F)c(OCc3ccon3)cc2[nH]1] | N-((5-fluoro-6- (isoxazol-3-ylmethoxy)- 1H-indol-2- yl)methyl)isobutyramide | 331.13 | 332.2 |
| 139 | 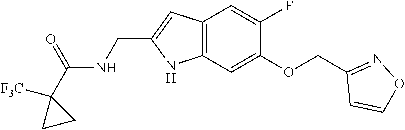 [SMILES: O=C(NCc1cc2cc(F)c(OCc3ccon3)cc2[nH]1)C1(C(F)(F)F)CC1] | N-((5-fluoro-6- (isoxazol-3-ylmethoxy)- 1H-indol-2-yl)methyl)- 1-(trifluoromethyl) cyclopropane- 1-carboxamide | 397.10 | 398.0 |
| 140 | 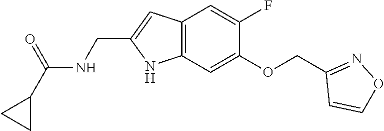 [SMILES: O=C(NCc1cc2cc(F)c(OCc3ccon3)cc2[nH]1)C1CC1] | N-((5-fluoro-6- (isoxazol-3-ylmethoxy)- 1H-indol-2- yl)methyl)cyclopropane carboxamide | 329.12 | 330.0 |
| 141 | 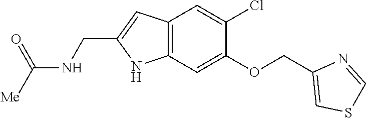 [SMILES: CC(=O)NCc1cc2cc(Cl)c(OCc3cscn3)cc2[nH]1] | N-((5-chloro-6-(thiazol- 4-ylmethoxy)-1H-indol- 2-yl)methyl)acetamide | 335.05 | 336.1 |
| 142 | 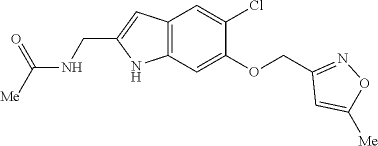 [SMILES: CC(=O)NCc1cc2cc(Cl)c(OCc3cc(C)on3)cc2[nH]1] | N-((5-chloro-6-((5- methylisoxazol-3- yl)methoxy)-1H-indol- 2-yl)methyl)acetamide | 333.09 | 334.0 |
| 143 | 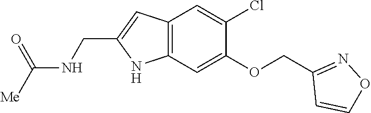 [SMILES: CC(=O)NCc1cc2cc(Cl)c(OCc3ccon3)cc2[nH]1] | N-((5-chloro-6- (isoxazol-3-ylmethoxy)- 1H-indol-2- yl)methyl)acetamide | 319.07 | 320.0 |
| 144 | 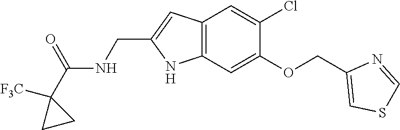 [SMILES: O=C(NCc1cc2cc(Cl)c(OCc3cscn3)cc2[nH]1)C1(C(F)(F)F)CC1] | N-((5-chloro-6-(thiazol- 4-ylmethoxy)-1H-indol- 2-yl)methyl)-1- (trifluoromethyl) cyclopropane-1- carboxamide | 429.05 | 429.9 |
| 145 | 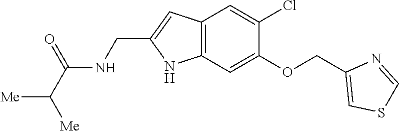 [SMILES: CC(C)C(=O)NCc1cc2cc(Cl)c(OCc3cscn3)cc2[nH]1] | N-((5-chloro-6-(thiazol- 4-ylmethoxy)-1H-indol- 2- yl)methyl)isobutyramide | 363.08 | 364.0 |
| 146 | 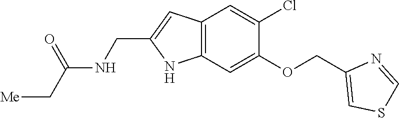 [SMILES: CCC(=O)NCc1cc2cc(Cl)c(OCc3cscn3)cc2[nH]1] | N-((5-chloro-6-(thiazol- 4-ylmethoxy)-1H-indol- 2- yl)methyl)propionamide | 349.07 | 350.1 |
| 147 | 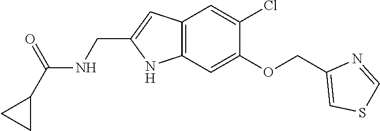 [SMILES: O=C(NCc1cc2cc(Cl)c(OCc3cscn3)cc2[nH]1)C1CC1] | N-((5-chloro-6-(thiazol- 4-ylmethoxy)-1H-indol- 2- yl)methyl)cyclopropane carboxamide | 361.07 | 361.9 |
| 237 | 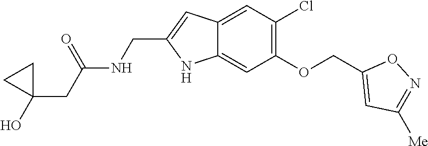 [SMILES: Cc1cc(COc2cc3[nH]c(CNC(=O)CC4(O)CC4)cc3cc2Cl)on1] | N-((5-chloro-6-((3- methylisoxazol-5- yl)methoxy)-1H-indol- 2-yl)methyl)-2-(1- hydroxycyclopropyl) acetamide | 389.11 | 390.1 |
| 238 | 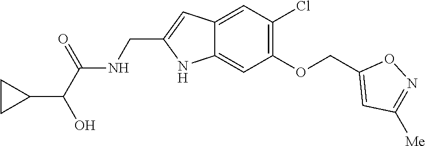 [SMILES: Cc1cc(COc2cc3[nH]c(CNC(=O)C(O)C4CC4)cc3cc2Cl)on1] | N-((5-chloro-6-((3- methylisoxazol-5- yl)methoxy)-1H-indol- 2-yl)methyl)-2- cyclopropyl-2- hydroxyacetamide | 389.11 | 390.0 |
| 239 | 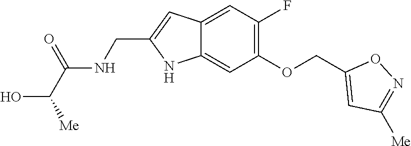 [SMILES: Cc1cc(COc2cc3[nH]c(CNC(=O)[C@H](C)O)cc3cc2F)on1] | (S)-N-((5-fluoro-6-((3- methylisoxazol-5- yl)methoxy)-1H-indol- 2-yl)methyl)-2- hydroxypropanamide | 347.13 | 348.3 |
| 240 | 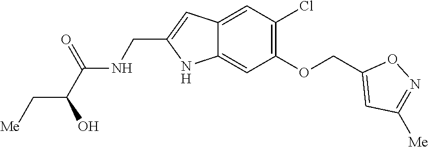 [SMILES: CC[C@H](O)C(=O)NCc1cc2cc(Cl)c(OCc3cc(C)no3)cc2[nH]1] | (S)-N-((5-chloro-6-((3- methylisoxazol-5- yl)methoxy)-1H-indol- 2-yl)methyl)-2- hydroxybutanamide | 377.11 | 378.1 |
| 241 | 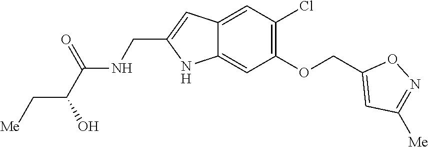 [SMILES: CC[C@@H](O)C(=O)NCc1cc2cc(Cl)c(OCc3cc(C)no3)cc2[nH]1] | (R)-N-((5-chloro-6-((3- methylisoxazol-5- yl)methoxy)-1H-indol- 2-yl)methyl)-2- hydroxybutanamide | 377.11 | 378.2 |
| 242 | 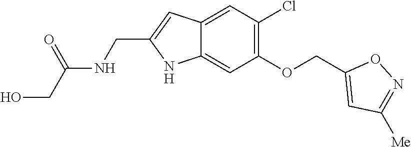 [SMILES: Cc1cc(COc2cc3[nH]c(CNC(=O)CO)cc3cc2Cl)on1] | N-((5-chloro-6-((3- methylisoxazol-5- yl)methoxy)-1H-indol- 2-yl)methyl)-2- hydroxyacetamide | 349.08 | 350.1 |
| 243 | 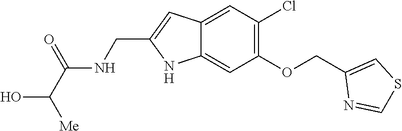 [SMILES: CC(O)C(=O)NCc1cc2cc(Cl)c(OCc3cscn3)cc2[nH]1] | N-((5-chloro-6-(thiazol- 4-ylmethoxy)-1H-indol- 2-yl)methyl)-2- hydroxypropanamide | 365.06 | 366.0 |
| 244 | 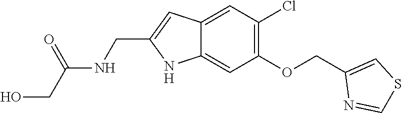 [SMILES: O=C(CO)NCc1cc2cc(Cl)c(OCc3cscn3)cc2[nH]1] | N-((5-chloro-6-(thiazol- 4-ylmethoxy)-1H-indol- 2-yl)methyl)-2- hydroxyacetamide | 351.04 | 352.0 |
| 245 | 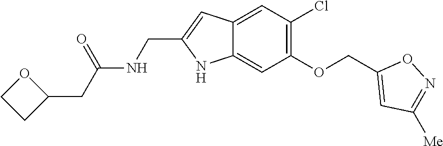 [SMILES: Cc1cc(COc2cc3[nH]c(CNC(=O)CC4CCO4)cc3cc2Cl)on1] | N-((5-chloro-6-((3- methylisoxazol-5- yl)methoxy)-1H-indol- 2-yl)methyl)-2-(oxetan- 2-yl)acetamide | 389.11 | 390.3 |
| 428 | 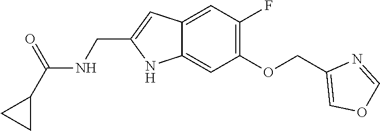 [SMILES: O=C(NCc1cc2cc(F)c(OCc3cocn3)cc2[nH]1)C1CC1] | N-((5-fluoro-6-(oxazol- 4-ylmethoxy)-1H-indol- 2- yl)methyl)cyclopropane carboxamide | 329.12 | 330.1 |
| 429 | 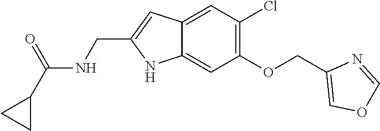 [SMILES: O=C(NCc1cc2cc(Cl)c(OCc3cocn3)cc2[nH]1)C1CC1] | N-((5-chloro-6-(oxazol- 4-ylmethoxy)-1H-indol- 2- yl)methyl)cyclopropane carboxamide | 345.09 | 346.1 |
| 430 | 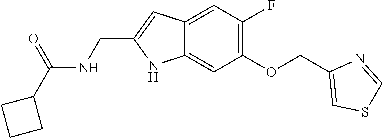 [SMILES: O=C(NCc1cc2cc(F)c(OCc3cscn3)cc2[nH]1)C1CCC1] | N-((5-fluoro-6-(thiazol- 4-ylmethoxy)-1H-indol- 2-yl)methyl) cyclobutanecarboxamide | 359.11 | 360.2 |
| 431 | 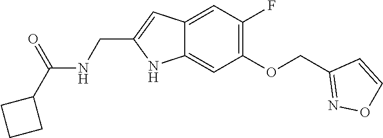 [SMILES: O=C(NCc1cc2cc(F)c(OCc3ccon3)cc2[nH]1)C1CCC1] | N-((5-fluoro-6- (isoxazol-3-ylmethoxy)- 1H-indol-2-yl)methyl) cyclobutanecarboxamide | 343.13 | 344.1 |
| 432 | 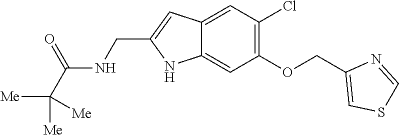 [SMILES: CC(C)(C)C(=O)NCc1cc2cc(Cl)c(OCc3cscn3)cc2[nH]1] | N-((5-chloro-6-(thiazol- 4-ylmethoxy)-1H-indol- 2-yl)methyl)pivalamide | 377.10 | 378.2 |
| 433 | 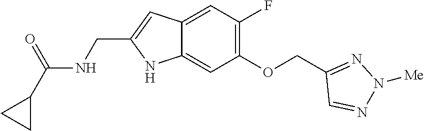 [SMILES: Cn1ncc(COc2cc3[nH]c(CNC(=O)C4CC4)cc3cc2F)n1] | N-((5-fluoro-6-((2- methyl-2H-1,2,3-triazol- 4-yl)methoxy)-1H- indol-2- yl)methyl)cyclopropane carboxamide | 343.14 | 344.3 |
| 434 | 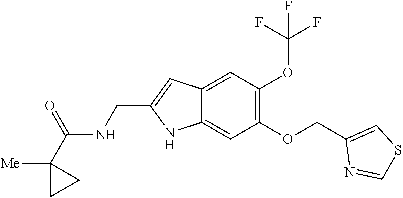 [SMILES: CC1(C(=O)NCc2cc3cc(OC(F)(F)F)c(OCc4cscn4)cc3[nH]2)CC1] | 1-methyl-N-((6-(thiazol- 4-ylmethoxy)-5- (trifluoromethoxy)-1H- indol-2- yl)methyl)cyclopropane- 1-carboxamide | 425.10 | 426.3 |
| 435 | 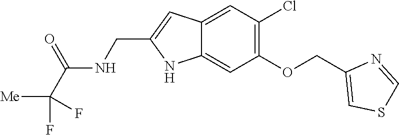 [SMILES: CC(F)(F)C(=O)NCc1cc2cc(Cl)c(OCc3cscn3)cc2[nH]1] | N-((5-chloro-6-(thiazol- 4-ylmethoxy)-1H-indol- 2-yl)methyl)-2,2- difluoropropanamide | 385.05 | 386.0 |
| 436 | 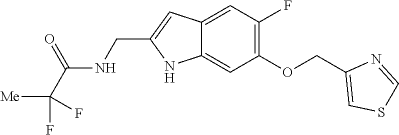 [SMILES: CC(F)(F)C(=O)NCc1cc2cc(F)c(OCc3cscn3)cc2[nH]1] | 2,2-difluoro-N-((5- fluoro-6-(thiazol-4- ylmethoxy)-1H-indol-2- yl)methyl)propanamide | 369.08 | 370.1 |
| 437 | 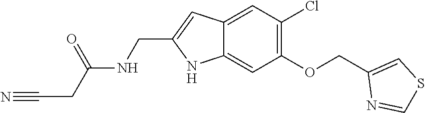 [SMILES: N#CCC(=O)NCc1cc2cc(Cl)c(OCc3cscn3)cc2[nH]1] | N-((5-chloro-6-(thiazol- 4-ylmethoxy)-1H-indol- 2-yl)methyl)-2- cyanoacetamide | 360.05 | 361.1 |
| 438 | 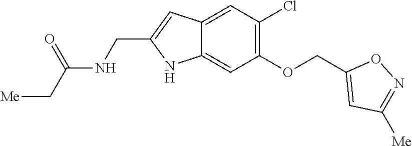 [SMILES: CCC(=O)NCc1cc2cc(Cl)c(OCc3cc(C)no3)cc2[nH]1] | N-((5-chloro-6-((3- methylisoxazol-5- yl)methoxy)-1H-indol- 2- yl)methyl)propionamide | 347.10 | 348.2 |
| 439 | 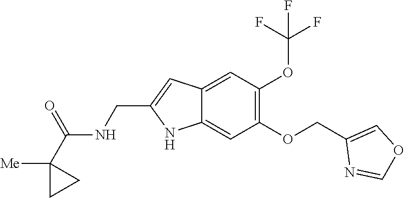 [SMILES: CC1(C(=O)NCc2cc3cc(OC(F)(F)F)c(OCc4cocn4)cc3[nH]2)CC1] | 1-methyl-N-((6-(oxazol- 4-ylmethoxy)-5- (trifluoromethoxy)-1H- indol-2- yl)methyl)cyclopropane- 1-carboxamide | 409.12 | 410.3 |
| 440 | 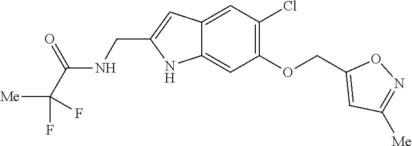 [SMILES: Cc1cc(COc2cc3[nH]c(CNC(=O)C(C)(F)F)cc3cc2Cl)on1] | N-((5-chloro-6-((3- methylisoxazol-5- yl)methoxy)-1H-indol- 2-yl)methyl)-2,2- difluoropropanamide | 383.08 | 384.1 |
| 441 | 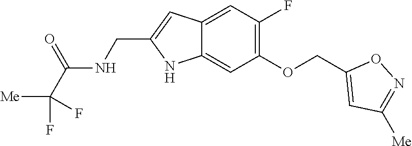 [SMILES: Cc1cc(COc2cc3[nH]c(CNC(=O)C(C)(F)F)cc3cc2F)on1] | 2,2-difluoro-N-((5- fluoro-6-((3- methylisoxazol-5- yl)methoxy)-1H-indol- 2- yl)methyl)propanamide | 367.11 | 368.1 |
| 442 | 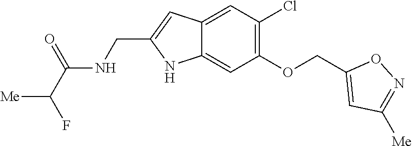 [SMILES: Cc1cc(COc2cc3[nH]c(CNC(=O)C(C)F)cc3cc2Cl)on1] | N-((5-chloro-6-((3- methylisoxazol-5- yl)methoxy)-1H-indol- 2-yl)methyl)-2- fluoropropanamide | 365.09 | 366.2 |
| 443 | 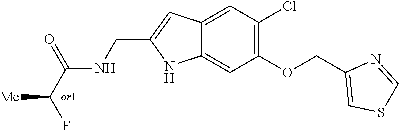 [SMILES: C[C@H](F)C(=O)NCc1cc2cc(Cl)c(OCc3cscn3)cc2[nH]1] | (S or R)-N-((5-chloro-6- (thiazol-4-ylmethoxy)- 1H-indol-2-yl)methyl)- 2-fluoropropanamide (SFC column: (S,S)- WHELK-O1, first eluting isomer) | 367.06 | 368.0 |
| 444 | 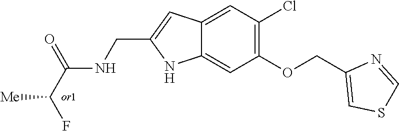 [SMILES: C[C@@H](F)C(=O)NCc1cc2cc(Cl)c(OCc3cscn3)cc2[nH]1] | (R or S)-N-((5-chloro-6- (thiazol-4-ylmethoxy)- 1H-indol-2-yl)methyl)- 2-fluoropropanamide (SFC column: (S,S)- WHELK-O1, second eluting isomer) | 367.06 | 368.1 |
| 445 | 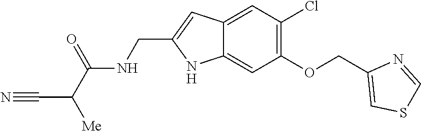 [SMILES: CC(C#N)C(=O)NCc1cc2cc(Cl)c(OCc3cscn3)cc2[nH]1] | N-((5-chloro-6-(thiazol- 4-ylmethoxy)-1H-indol- 2-yl)methyl)-2- cyanopropanamide | 374.06 | 375.1 |
| 446 | 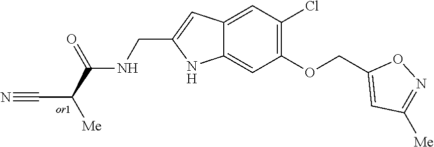 [SMILES: Cc1cc(COc2cc3[nH]c(CNC(=O)[C@@H](C)C#N)cc3cc2Cl)on1] | (R or S)-N-((5-chloro-6- ((3-methylisoxazol-5- yl)methoxy)-1H-indol- 2-yl)methyl)-2- cyanopropanamide (SFC column: Chiralcel OJ-3, first eluting isomer) | 372.10 | 373.0 |
| 447 | 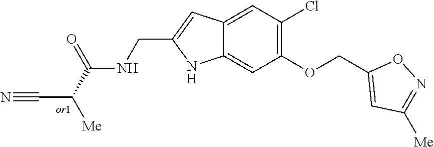 [SMILES: Cc1cc(COc2cc3[nH]c(CNC(=O)[C@H](C)C#N)cc3cc2Cl)on1] | (S or R)-N-((5-chloro-6- ((3-methylisoxazol-5- yl)methoxy)-1H-indol- 2-yl)methyl)-2- cyanopropanamide (SFC column: Chiralcel OJ-3, second eluting isomer) | 372.10 | 373.0 |
| 448 | 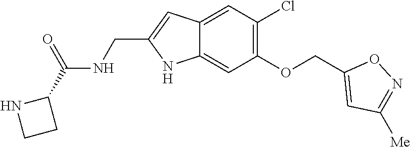 [SMILES: Cc1cc(COc2cc3[nH]c(CNC(=O)[C@@H]4CCN4)cc3cc2Cl)on1] | (S)-N-((5-chloro-6-((3- methylisoxazol-5- yl)methoxy)-1H-indol- 2-yl)methyl)azetidine-2- carboxamide | 374.11 | 375.0 |
| 449 | 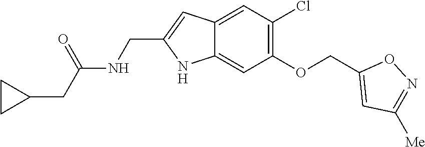 [SMILES: Cc1cc(COc2cc3[nH]c(CNC(=O)CC4CC4)cc3cc2Cl)on1] | N-((5-chloro-6-((3- methylisoxazol-5- yl)methoxy)-1H-indol- 2-yl)methyl)-2- cyclopropylacetamide | 373.12 | 374.1 |
| 450 | 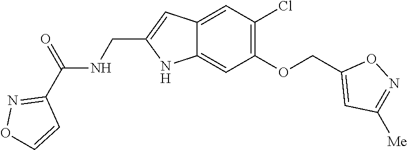 [SMILES: Cc1cc(COc2cc3[nH]c(CNC(=O)c4ccon4)cc3cc2Cl)on1] | N-((5-chloro-6-((3- methylisoxazol-5- yl)methoxy)-1H-indol- 2-yl)methyl)isoxazole- 3-carboxamide | 386.08 | 387.2 |
| 451 | 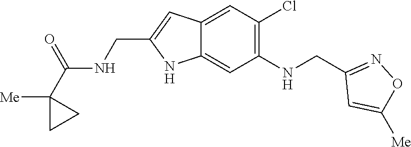 [SMILES: Cc1cc(CNc2cc3[nH]c(CNC(=O)C4(C)CC4)cc3cc2Cl)no1] | N-((5-chloro-6-(((5- methylisoxazol-3- yl)methyl)amino)-1H- indol-2-yl)methyl)-1- methylcyclopropane-1- carboxamide | 372.14 | 373.3 |
| 452 | 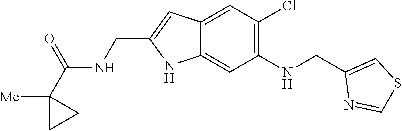 [SMILES: CC1(C(=O)NCc2cc3cc(Cl)c(NCc4cscn4)cc3[nH]2)CC1] | N-((5-chloro-6-((thiazol- 4-ylmethyl)amino)-1H- indol-2-yl)methyl)-1- methylcyclopropane-1- carboxamide | 374.10 | 375.2 |
| 453 | 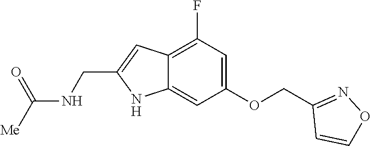 [SMILES: CC(=O)NCc1cc2c(F)cc(OCc3ccon3)cc2[nH]1] | N-((4-fluoro-6- (isoxazol-3-ylmethoxy)- 1H-indol-2- yl)methyl)acetamide | 303.10 | 304.1 |

### Example 22: (Compound 148)

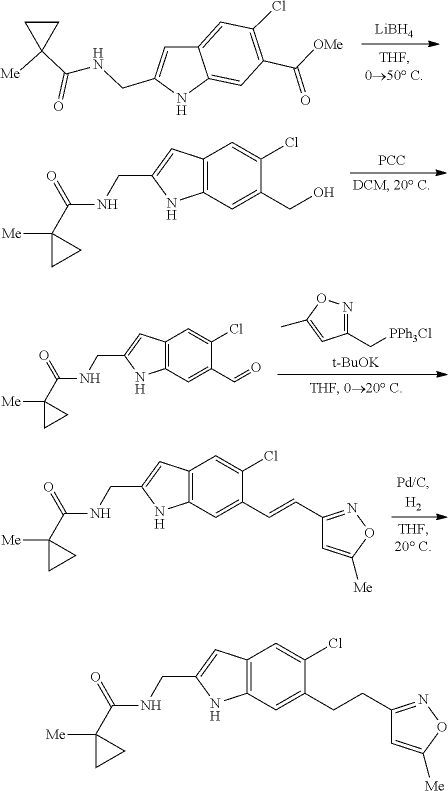

### Step 1: N-((5-chloro-6-(hydroxymethyl)-1H-indol-2-yl)methyl)-1-methylcyclopropanecarboxamide

Two reactions were carried out in parallel. To a solution of methyl 5-chloro-2-((1-methylcyclopropanecarboxamido)methyl)-1H-indole-6-carboxylate (1.00 g, 3.12 mmol, 1.00 eq) in THF (10 mL) at 0° C. was added LiBH₄ (4 M in THF, 4.68 mL, 18.7 mmol, 6.00 eq) dropwise under N₂. The mixture was stirred at 0° C. for 0.5 h, then was heated to 50° C. and stirred for 12 h. The two parallel reactions were combined, poured into water (10 mL) and stirred for 2 min. The aqueous phase was extracted with ethyl acetate (3×20 mL). The combined organic phase was washed with brine (2×10 mL), dried with anhydrous Na₂SO₄, filtered, and concentrated under reduced pressure. Purification by column chromatography gave N-((5-chloro-6-(hydroxymethyl)-1H-indol-2-yl)methyl)-1-methylcyclopropanecarboxamide. LC-MS (ESI) m/z calcd for C₁₅H₁₇ClN₂O₂: 292.10; found 293.2 [M+H]⁺.

### Step 2: N-((5-chloro-6-formyl-1H-indol-2-yl)methyl)-1-methylcyclopropanecarboxamide

To a solution of N-((5-chloro-6-(hydroxymethyl)-1H-indol-2-yl)methyl)-1-methylcyclopropanecarboxamide (500 mg, 1.71 mmol, 1.00 eq) in DCM (5 mL) was added PCC (552 mg, 2.56 mmol, 1.50 eq), and the mixture was stirred for 1 h. The reaction mixture was poured into ice-water (5 mL) and stirred for 2 min. The aqueous phase was extracted with ethyl acetate (3×15 mL). The combined organic phase was washed with brine (2×10 mL), dried over anhydrous Na₂SO₄, filtered, and concentrated under reduced pressure. The crude product was triturated with MTBE (5 mL) at 20° C. for 10 min and then filtered. The filter cake was collected to give N-((5-chloro-6-formyl-1H-indol-2-yl)methyl)-1-methylcyclopropanecarboxamide. LC-MS (ESI) m/z calcd for C₁₅H₁₅ClN₂O₂: 290.08; found 291.1 [M+H]⁺.

### Step 3: (E)-N-((5-chloro-6-(2-(5-methylisoxazol-3-yl)vinyl)-1H-indol-2-yl)methyl)-1-methylcyclopropanecarboxamide

To a solution of 3-((chlorotriphenylphosphoranyl)methyl)-5-methylisoxazole (634 mg, 1.61 mmol, 1.30 eq) in THF (6 mL) at 0° C. was added t-BuOK (1 M in THF, 2.23 mL, 2.23 mmol, 1.80 eq) dropwise. N-((5-chloro-6-formyl-1H-indol-2-yl)methyl)-1-methylcyclopropanecarboxamide (360 mg, 1.24 mmol, 1.00 eq) was then added, and the resulting mixture was stirred at 20° C. for 1 h. The mixture was quenched by pouring into ice water (5 mL) and stirred for 2 min. The aqueous phase was extracted with ethyl acetate (3×15 mL). The combined organic phase was washed with brine (2×10 mL), dried with anhydrous Na₂SO₄, filtered, and concentrated under reduced pressure. The crude material was purified by column chromatography followed by trituration with MTBE (3 mL at 20° C. for 5 min). The mixture was then filtered, and the filter cake was collected to give (E)-N-((5-chloro-6-(2-(5-methylisoxazol-3-yl)vinyl)-1H-indol-2-yl)methyl)-1-methylcyclopropanecarboxamide. LC-MS (ESI) m/z calcd for C₂₀H₂₀ClN₃O₂: 369.12; found 370.2 [M+H]⁺.

### Step 4: N-((5-chloro-6-(2-(5-methylisoxazol-3-yl)ethyl)-1H-indol-2-yl)methyl)-1-methylcyclopropanecarboxamide

To a solution of (E)-N-((5-chloro-6-(2-(5-methylisoxazol-3-yl)vinyl)-1H-indol-2-yl)methyl)-1-methylcyclopropanecarboxamide (240 mg, 649 μmol, 1.00 eq) in THF (3 mL) was added Pd/C (80 mg, 649 μmol, 10% by weight, 1.00 eq) under N₂. The suspension was degassed under vacuum and purged with H₂ several times, then was stirred under H₂ (15 psi) for 20 min. The reaction mixture was filtered through Celite, and the filtrate was concentrated under reduced pressure. Purification by prep-HPLC gave N-((5-chloro-6-(2-(5-methylisoxazol-3-yl)ethyl)-1H-indol-2-yl)methyl)-1-methylcyclopropanecarboxamide (Compound 148). LC-MS (ESI) m/z calcd for C₂₀H₂₂ClN₃O₂: 371.14; found 372.1 [M+H]⁺. ¹H NMR (400 MHz, DMSO-d₆) δ 10.89 (s, 1H), 8.03 (t, J=5.6 Hz, 1H), 7.50 (s, 1H), 7.28 (s, 1H), 6.17 (s, 1H), 6.15 (s, 1H), 4.37 (d, J=5.6 Hz, 2H), 3.07-3.03 (m, 2H), 2.89-2.85 (m, 2H), 2.35 (s, 3H), 1.29 (s, 3H), 1.01-0.98 (m, 2H), 0.55-0.52 (m, 2H).

The following compounds in Table T-14 were synthesized using procedures similar to Compound 148 using the appropriate starting materials.

TABLE T-14

| | | | Exact | LCMS, |
|---|---|---|---|---|
| Compound | | | mass | found |
| No. | Structure | IUPAC name | (g/mol) | [M + H]⁺ |
| 149 | 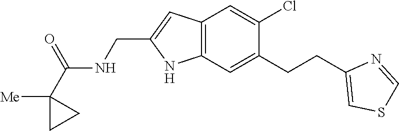 [SMILES: CC1(C(=O)NCc2cc3cc(Cl)c(CCc4cscn4)cc3[nH]2)CC1] | N-((5-chloro-6-(2- (thiazol-4-yl)ethyl)- 1H-indol-2- yl)methyl)-1- methylcyclopropane- 1-carboxamide | 373.10 | 374.1 |
| 150 | 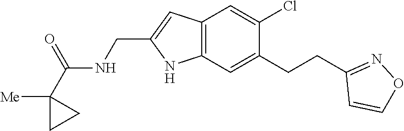 [SMILES: CC1(C(=O)NCc2cc3cc(Cl)c(CCc4ccon4)cc3[nH]2)CC1] | N-((5-chloro-6-(2- (isoxazol-3- yl)ethyl)-1H-indol- 2-yl)methyl)-1- methylcyclopropane- 1-carboxamide | 357.12 | 358.1 |

### Example 23: (Compound 151)

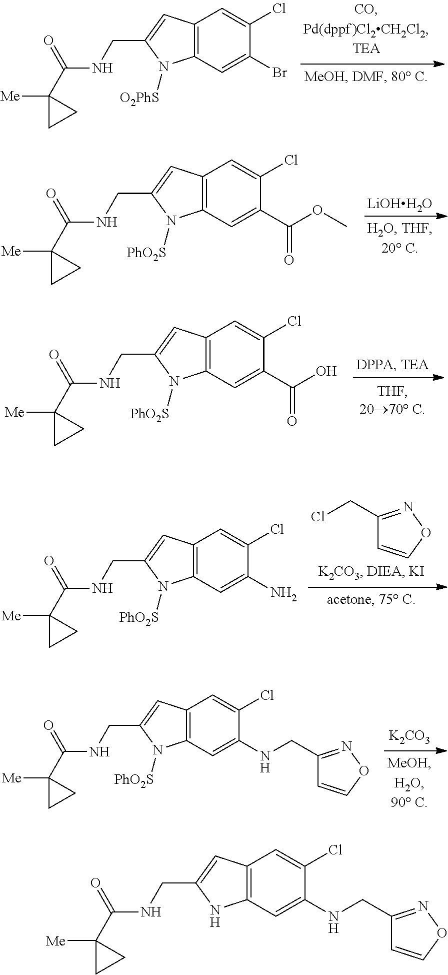

### Step 1: methyl 5-chloro-2-((1-methylcyclopropane-1-carboxamido)methyl)-1-(phenylsulfonyl)-1H-indole-6-carboxylate

To a solution of N-((6-bromo-5-chloro-1-(phenylsulfonyl)-1H-indol-2-yl)methyl)-1-methylcyclopropane-1-carboxamide (3.40 g, 7.06 mmol, 1.00 eq) in MeOH (30 mL) and DMF (6 mL) were added TEA (9.88 mmol, 1.38 mL, 1.40 eq) and Pd(dppf)Cl₂·CH₂Cl₂ (576 mg, 705 μmol, 0.10 eq) under N₂. The reaction was pressurized to 30 psi with CO and stirred for 16 h at 80° C. The reaction mixture was poured into ice water (40 mL) and stirred for 3 min. The aqueous phase was extracted with ethyl acetate (3×30 mL). The combined organic phase was washed with brine (30 mL), dried over anhydrous Na₂SO₄, filtered, and concentrated under reduced pressure. Purification by column chromatography gave methyl 5-chloro-2-((1-methylcyclopropane-1-carboxamido)methyl)-1-(phenylsulfonyl)-1H-indole-6-carboxylate. LC-MS (ESI) m/z calcd for C₂₂H₂₁ClN₂O₅S: 460.09; found 460.9 [M+H]⁺.

### Step 2: 5-chloro-2-((1-methylcyclopropane-1-carboxamido)methyl)-1-(phenylsulfonyl)-1H-indole-6-carboxylic acid

To a solution of methyl 5-chloro-2-((1-methylcyclopropane-1-carboxamido)methyl)-1-(phenylsulfonyl)-1H-indole-6-carboxylate (2.70 g, 5.86 mmol, 1.00 eq) in H₂O (10 mL) and THF (20 mL) was added LiOH·H₂O (295 mg, 7.03 mmol, 1.20 eq), and the mixture was stirred at for 16 h. The reaction mixture was then diluted with water (30 mL) and extracted with dichloromethane (2×25 mL). The aqueous phase was adjusted to pH 5-6 by addition of saturated aqueous citric acid, then was extracted with dichloromethane (2×25 mL). The combined organic phase was concentrated in vacuo to give 5-chloro-2-((1-methylcyclopropane-1-carboxamido)methyl)-1-(phenylsulfonyl)-1H-indole-6-carboxylic acid, which was used without further purification. LC-MS (ESI) m/z calcd for C₂₁H₁₉ClN₂05S: 446.07; found 446.9 [M+H]⁺.

### Step 3: N-((6-amino-5-chloro-1-(phenylsulfonyl)-1H-indol-2-yl)methyl)-1-methylcyclopropane-1-carboxamide

To a solution of 5-chloro-2-((1-methylcyclopropane-1-carboxamido)methyl)-1-(phenylsulfonyl)-1H-indole-6-carboxylic acid (1.89 g, 4.23 mmol, 1.00 eq) in THF (20 mL) were added TEA (6.34 mmol, 883 μL, 1.50 eq) and DPPA (1.01 mL, 4.65 mmol, 1.10 eq). The mixture was stirred for 3 h at RT, then was heated to 70° C. and stirred for 1 h. H₂O (7.6 mL) was then added and the resulting mixture was stirred at 70° C. for 3 h. The reaction mixture was allowed to cooled to room temperature, then was poured into ice water (20 mL) and stirred for 3 min. The aqueous phase was extracted with ethyl acetate (3×30 mL). The combined organic phase was washed with brine (40 mL), dried over anhydrous Na₂SO₄, filtered, and concentrated under reduced pressure. Purification by column chromatography gave N-((6-amino-5-chloro-1-(phenylsulfonyl)-1H-indol-2-yl)methyl)-1-methylcyclopropane-1-carboxamide. LC-MS (ESI) m/z calcd for C₂₀H₂₀ClN₃O₃S: 417.09; found 418.2 [M+H]⁺.

### Step 4: N-((5-chloro-6-((isoxazol-3-ylmethyl)amino)-1-(phenylsulfonyl)-1H-indol-2-yl)methyl)-1-methylcyclopropane-1-carboxamide

Two reactions were carried out in parallel. To a solution of N-((6-amino-5-chloro-1-(phenylsulfonyl)-1H-indol-2-yl)methyl)-1-methylcyclopropane-1-carboxamide (100 mg, 260 μmol, 1.00 eq) in acetone (5 mL) were added 3-(chloromethyl)isoxazole (61.3 mg, 521 μmol, 2.00 eq), K₂CO₃ (90.0 mg, 652 μmol, 2.50 eq), DIEA (67.0 mg, 521 μmol, 2.00 eq) and KI (21.0 mg, 130 μmol, 0.50 eq). The mixture was heated to 75° C. and stirred for 16 h. The two reactions were allowed to cool to RT, then were combined, poured into ice water (10 mL), and stirred for 3 min. The aqueous phase was extracted with ethyl acetate (3×10 mL). The combined organic phase was washed with brine (10 mL), dried over anhydrous Na₂SO₄, filtered, and concentrated under reduced pressure. Purification by prep-TLC gave N-((5-chloro-6-((isoxazol-3-ylmethyl)amino)-1-(phenylsulfonyl)-1H-indol-2-yl)methyl)-1-methylcyclopropane-1-carboxamide. LC-MS (ESI) m/z calcd for C₂₄H₂₃ClN₄04S: 498.11; found 499.1 [M+H]⁺.

### Step 5: N-((5-chloro-6-((isoxazol-3-ylmethyl)amino)-1H-indol-2-yl)methyl)-1-methylcyclopropane-1-carboxamide

To a solution of N-((5-chloro-6-((isoxazol-3-ylmethyl)amino)-1-(phenylsulfonyl)-1H-indol-2-yl)methyl)-1-methylcyclopropane-1-carboxamide (280 mg, 561 μmol, 1.00 eq) in MeOH (3 mL) and H₂O (1 mL) was added K₂CO₃ (370 mg, 2.68 mmol, 4.78 eq). The mixture was heated to 90° C. and stirred for 16 h. The reaction mixture was then poured into ice water (5 mL) and stirred for 3 min. The aqueous phase was extracted with ethyl acetate (3×10 mL). The combined organic phase was washed with brine (5 mL), dried over anhydrous Na₂SO₄, filtered, and concentrated under reduced pressure. Purification by prep-HPLC gave N-((5-chloro-6-((isoxazol-3-ylmethyl)amino)-1H-indol-2-yl)methyl)-1-methylcyclopropane-1-carboxamide (Compound 151). LC-MS (ESI) m/z calcd for C₁₈H₁₉ClN₄O₂: 358.12; found 359.1 [M+H]⁺. ¹H NMR (400 MHz, DMSO-d₆) δ 10.49 (s, 1H), 8.78 (d, J=1.6 Hz, 1H), 7.91 (t, J=5.6 Hz, 1H), 7.35 (s, 1H), 6.62 (s, 1H), 6.47 (s, 1H), 5.99 (s, 1H), 5.65 (t, J=6.2 Hz, 1H), 4.45 (d, J=6.4 Hz, 2H), 4.29 (d, J=6.4 Hz, 2H), 1.27 (s, 3H), 1.00-0.96 (m, 2H), 0.52-0.50 (m, 2H).

### Example 24: (Compound 152)

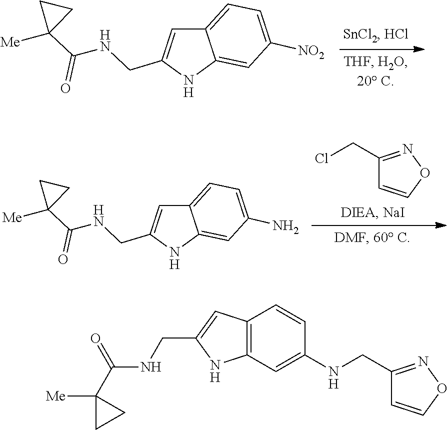 [SMILES: CC1(C(=O)NCc2cc3ccc([N+](=O)[O-])cc3[nH]2)CC1 ; CC1(C(=O)NCc2cc3ccc(N)cc3[nH]2)CC1 ; ClCc1ccon1 ; CC1(C(=O)NCc2cc3ccc(NCc4ccon4)cc3[nH]2)CC1]

### Step 1: N-((6-amino-1H-indol-2-yl)methyl)-1-methylcyclopropane-1-carboxamide

To a solution of 1-methyl-N-((6-nitro-1H-indol-2-yl)methyl)cyclopropane-1-carboxamide (900 mg, 3.29 mmol, 1.00 eq) in saturated aqueous HCl (4 mL) and THF (1 mL) was added SnCl₂ (16.4 mmol, 3.12 g, 5.00 eq) in one portion. The mixture was stirred at 20° C. for 1 h under N₂. The reaction mixture was then quenched by addition H₂O (10 mL) and extracted with EtOAc (3×10 mL). The combined organic layers were dried over Na₂SO₄, filtered, and concentrated under reduced pressure. Purification by column chromatography gave N-((6-amino-1H-indol-2-yl)methyl)-1-methylcyclopropane-1-carboxamide. LC-MS (ESI) m/z calcd for C₁₄H₁₇N₃O: 243.14; found 244.2 [M+H]⁺.

### Step 2: N-((6-((isoxazol-3-ylmethyl)amino)-1H-indol-2-yl)methyl)-1-methylcyclopropane-1-carboxamide

To a solution of 3-(chloromethyl)isoxazole (38.6 mg, 328 μmol, 0.80 eq) in DMF (1 mL) were added DIEA (822 μmol, 143 uL, 2.00 eq), followed by NaI (123 mg, 822 μmol, 2.00 eq), and then N-((6-amino-1H-indol-2-yl)methyl)-1-methylcyclopropane-1-carboxamide (100 mg, 411 μmol, 1.00 eq). The resulting mixture was heated to 60° C. and stirred for 2 h. The reaction mixture was allowed to cool to room temperature, then was quenched by addition H₂O (5 mL) and extracted with DCM (3×5 mL). The combined organic layers were dried over Na₂SO₄, filtered, and concentrated under reduced pressure. Purification by prep-HPLC gave N-((6-((isoxazol-3-ylmethyl)amino)-1H-indol-2-yl)methyl)-1-methylcyclopropane-1-carboxamide (Compound 152). LC-MS (ESI) m/z calcd for C₁₈H₂₀N₄O₂: 324.16; found 325.2 [M+H]⁺. ¹H NMR (400 MHz, DMSO-d₆) δ 10.25 (s, 1H), 8.76 (s, 1H), 7.87 (t, J=5.6 Hz, 1H), 7.11 (d, J=8.4 Hz, 1H), 6.47 (d, J=15.2 Hz, 2H), 6.44-6.41 (m, 1H), 5.94 (s, 1H), 5.82 (t, J=6.0 Hz, 1H), 4.32-4.28 (m, 4H), 1.27 (s, 3H), 0.99-0.94 (m, 2H), 0.52-0.49 (m, 2H).

### Example 25: (Compound 153)

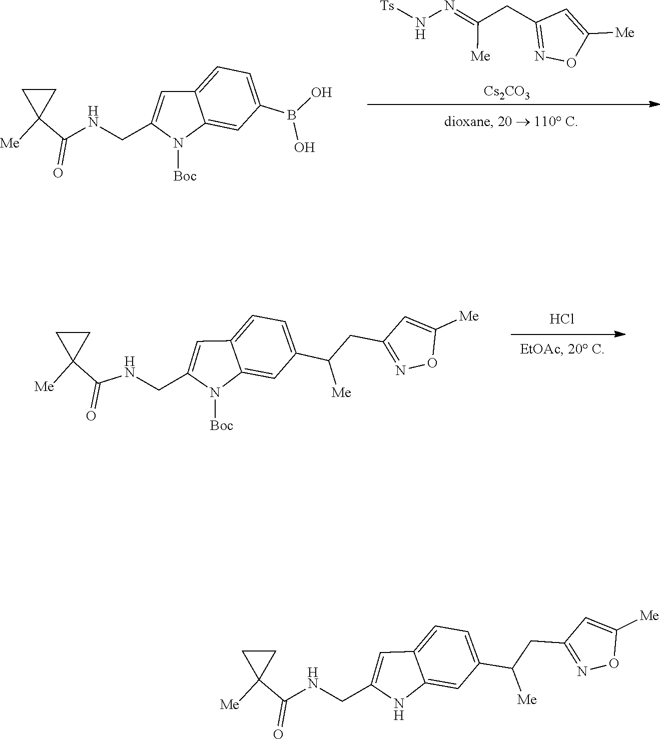 [SMILES: C/C(Cc1cc(C)on1)=N\NS(=O)(=O)c1ccc(C)cc1 ; CC(C)(C)OC(=O)n1c(CNC(=O)C2(C)CC2)cc2ccc(B(O)O)cc21 ; "Cs2CO3" ; Cc1cc(CC(C)c2ccc3cc(CNC(=O)C4(C)CC4)n(C(=O)OC(C)(C)C)c3c2)no1 ; Cl ; Cc1cc(CC(C)c2ccc3cc(CNC(=O)C4(C)CC4)[nH]c3c2)no1]

### Step 1: tert-butyl 2-((1-methylcyclopropane-1-carboxamido) methyl)-6-(1-(5-methylisoxazol-3-yl) propan-2-yl)-1H-indole-1-carboxylate

To a mixture of (1-(tert-butoxycarbonyl)-2-((1-methylcyclopropane-1-carboxamido) methyl)-1H-indol-6-yl)boronic acid (150 mg, 402 μmol, 1.00 eq) and 4-methyl-N′-(1-(5-methylisoxazol-3-yl)propan-2-ylidene)benzenesulfonohydrazide (123 mg, 402 μmol, 1.00 eq) in dioxane (10 mL) was added Cs₂CO₃ (328 mg, 1.01 mmol, 2.50 eq) in one portion at 20° C. under N₂. The resulting mixture was heated to 110° C. and stirred for 1 h. After allowing the mixture to cool to room temperature, the reaction was quenched by addition of H₂O (20 mL) and extracted with ethyl acetate (3×20 mL). The combined organic layers were dried over Na₂SO₄, filtered, and concentrated under reduced pressure to give tert-butyl 2-((1-methylcyclopropane-1-carboxamido)methyl)-6-(1-(5-methylisoxazol-3-yl)propan-2-yl)-1H-indole-1-carboxylate, which was used without further purification. LC-MS (ESI) m/z calcd for C₂₆H₃₃N₃O₄: 451.25; found 452.3 [M+H]⁺.

### Step 2: 1-methyl-N-((6-(1-(5-methylisoxazol-3-yl) propan-2-yl)-1H-indol-2-yl) methyl) cyclopropane-1-carboxamide

To a solution of tert-butyl 2-((1-methylcyclopropane-1-carboxamido) methyl)-6-(1-(5-methylisoxazol-3-yl) propan-2-yl)-1H-indole-1-carboxylate (80.0 mg, 177 μmol, 1.00 eq) in ethyl acetate (EtOAc, 1 mL) was added HCl (4M in EtOAc, 2 mL, 8.00 mmol, 8 eq) in one portion under N₂. The mixture was stirred for 0.5 h, then was diluted with H₂O (30 mL) and extracted with ethyl acetate (3×10 mL). The combined organic layers were dried over Na₂SO₄, filtered, and concentrated under reduced pressure. Purification by prep-TLC gave 1-methyl-N-((6-(1-(5-methylisoxazol-3-yl) propan-2-yl)-1H-indol-2-yl) methyl) cyclopropane-1-carboxamide (Compound 153). LC-MS (ESI) m/z calcd for C₂₁H₂₅N₃O₂: 351.19; found 352.2 [M+H]⁺. ¹H NMR (400 MHz, DMSO-d₆) δ 10.62 (s, 1H), 7.98 (t, J=5.6 Hz, 1H), 7.32 (d, J=8.0 Hz, 1H), 7.17 (s, 1H), 6.87 (d, J=9.6 Hz, 1H), 6.12 (s, 1H), 5.95 (s, 1H), 4.37 (t, J=6.4 Hz, 2H), 3.10-3.15 (m, 1H), 2.91-2.82 (m, 2H), 2.28 (s, 3H), 1.28 (s, 3H), 1.22 (d, J=6.8 Hz, 3H), 0.97-0.99 (m, 2H), 0.51-0.53 (m, 2H).

### Example 26: (Compound 154)

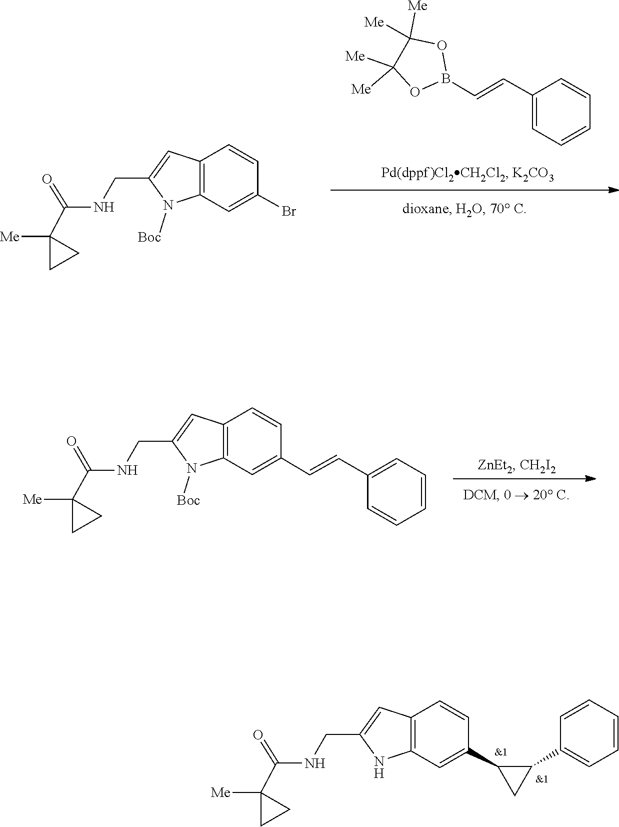 [SMILES: CC(C)(C)OC(=O)n1c(CNC(=O)C2(C)CC2)cc2ccc(Br)cc21 ; CC1(C)OB(/C=C/c2ccccc2)OC1(C)C ; CC(C)(C)OC(=O)n1c(CNC(=O)C2(C)CC2)cc2ccc(/C=C/c3ccccc3)cc21 ; CC1(C(=O)NCc2cc3ccc([C@@H]4C[C@H]4c4ccccc4)cc3[nH]2)CC1]

### Step 1: tert-butyl (E)-2-((1-methylcyclopropane-1-carboxamido)methyl)-6-styryl-1H-indole-1-carboxylate

To a solution of tert-butyl 6-bromo-2-((1-methylcyclopropane-1-carboxamido)methyl)-1H-indole-1-carboxylate (500 mg, 1.23 mmol, 1.00 eq) and (E)-4,4,5,5-tetramethyl-2-styryl-1,3,2-dioxaborolane (395 mg, 1.72 mmol, 1.40 eq) in dioxane (0.4 mL) and H₂O (0.1 mL) were added K₂CO₃ (237 mg, 1.72 mmol, 1.40 eq) and Pd(dppf)Cl₂·CH₂Cl₂ (100 mg, 122 μmol, 0.1 eq) under N₂ and the resulting mixture was heated to 70° C. and stirred for 5 h. Upon cooling to room temperature, the reaction mixture was quenched by addition H₂O (10 mL) and extracted with EtOAc (3×15 mL). The combined organic layers were dried over Na₂SO₄, filtered, and concentrated under reduced pressure. Purification by column chromatography gave tert-butyl (E)-2-((1-methylcyclopropane-1-carboxamido)methyl)-6-styryl-1H-indole-1-carboxylate (500 mg). LC-MS (ESI) m/z calcd for C₂₇H₃₀N₂O₃: 430.23; found 431.3 [M+H]⁺.

### Step 2: trans-1-methyl-N-((6-(2-phenylcyclopropyl)-1H-indol-2-yl)methyl)cyclopropane-1-carboxamide

To a solution of ZnEt₂ (1 M in hexanes, 6.16 mL, 6.16 mmol, 10.0 eq) in DCM (3 mL) at 0° C. under a nitrogen atmosphere was added dropwise CH₂I₂ (6.16 mmol, 496 μL, 10.0 eq). After 0.5 h, tert-butyl (E)-2-((1-methylcyclopropane-1-carboxamido)methyl)-6-styryl-1H-indole-1-carboxylate (265 mg, 615 μmol, 1.00 eq) was added. The mixture was allowed to warm to room temperature and stirred for 3 h. The reaction mixture was poured into a saturated aq. NH₄Cl solution (10 mL) at 0° C., then was extracted with EtOAc (3×15 mL). The combined organic layers were dried over Na₂SO₄, filtered, and concentrated under reduced pressure. Purification by column chromatography, followed by additional purification by prep-HPLC gave trans-1-methyl-N-((6-(2-phenylcyclopropyl)-1H-indol-2-yl)methyl)cyclopropane-1-carboxamide (Compound 154). LC-MS (ESI) m/z calcd for C₂₃H₂₄N₂O: 344.19; found 345.3 [M+H]⁺. ¹H NMR (400 MHz, DMSO-d₆) δ 10.61 (s, 1H), 7.98-8.00 (t, J=5.6 Hz, 1H), 7.34-7.32 (m, 1H), 7.27-7.25 (m, 2H), 7.17-7.15 (m, 4H), 6.77 (d, J=8.0 Hz, 1H), 6.14 (s, 1H), 4.40-4.37 (m, 2H), 2.24-2.22 (m, 1H), 2.13-2.10 (m, 1H), 1.44-1.41 (m, 2H), 1.29 (s, 3H), 1.00-0.98 (m, 2H), 0.54-0.52 (m, 2H).

### Example 27: (Compound 155)

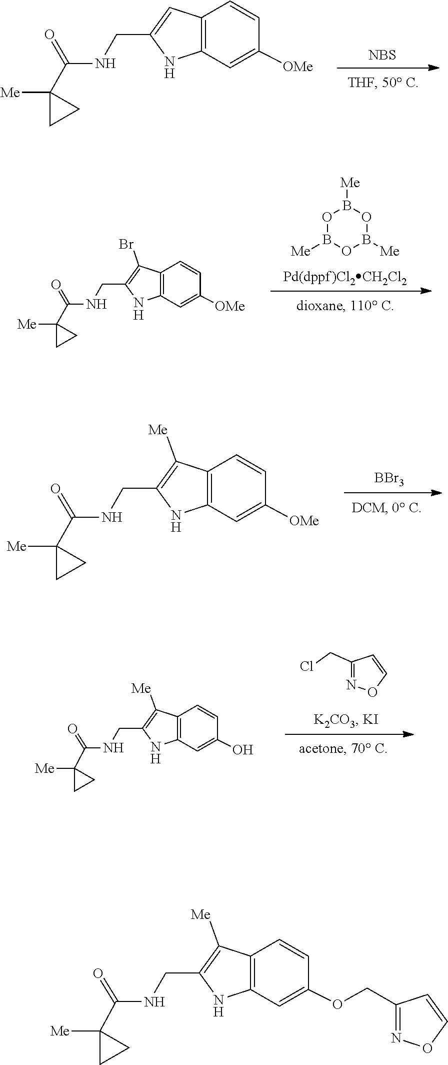

### Step 1: N-((3-bromo-6-methoxy-1H-indol-2-yl)methyl)-1-methylcyclopropane-1-carboxamide

To a solution of N-((6-methoxy-1H-indol-2-yl)methyl)-1-methylcyclopropane-1-carboxamide (3.20 g, 12.3 mmol, 1.00 eq) in THF (40 mL) was added NBS (2.20 g, 12.3 mmol, 1.00 eq), and the mixture was heated to 50° C. and stirred for 0.5 h. The reaction mixture was quenched by addition of saturated aq. NaHSO₃ (10 mL) and extracted with EtOAc (3×15 ml). The organic layer was dried over Na₂SO₄, filtered, and concentrated under reduced pressure. Purification by column chromatography gave N-((3-bromo-6-methoxy-1H-indol-2-yl)methyl)-1-methylcyclopropane-1-carboxamide. LC-MS (ESI) m/z calcd for C₁₅H₁₇BrN₂O₂: 336.05; found: 337.1 [M+H]⁺.

### Step 2: N-((6-methoxy-3-methyl-1H-indol-2-yl)methyl)-1-methylcyclopropane-1-carboxamide

To a solution of N-((3-bromo-6-methoxy-1H-indol-2-yl)methyl)-1-methylcyclopropane-1-carboxamide (700 mg, 2.08 mmol, 1.00 eq) and trimethylboroxine (8.30 mmol, 2.32 mL, 50% by weight, 4.00 eq) in dioxane (10 mL) were added Pd(dppf)Cl₂·CH₂Cl₂ (169 mg, 207 μmol, 0.1 eq) and Cs₂CO₃ (2.03 g, 6.23 mmol, 3.00 eq) under N₂. The mixture was heated to 110° C. and stirred for 1 h. The reaction mixture allowed to cool to room temperature, then was quenched by addition of H₂O (10 mL) and extracted with EtOAc (3×15 ml). The combined organic layers were dried over Na₂SO₄, filtered, and concentrated under reduced pressure. Purification by column chromatography gave N-((6-methoxy-3-methyl-1H-indol-2-yl)methyl)-1-methylcyclopropane-1-carboxamide. LC-MS (ESI) m/z calcd for C₁₆H₂₀N₂O₂: 272.15; found: 273.2 [M+H]⁺.

### Step 3: N-((6-hydroxy-3-methyl-1H-indol-2-yl)methyl)-1-methylcyclopropane-1-carboxamide

To a solution of N-((6-methoxy-3-methyl-1H-indol-2-yl)methyl)-1-methylcyclopropane-1-carboxamide (800 mg, 2.94 mmol, 1.00 eq) in DCM (10 mL) at 0° C. was added BBr₃ (11.7 mmol, 1.10 mL, 4.00 eq) dropwise, and the mixture was stirred at 0° C. for 2 h. The reaction mixture was then poured into water (50 mL) and stirred for 5 min. The aqueous phase was adjusted to pH 8 by addition of NaHCO₃, then was extracted with ethyl acetate (2×200 mL). The combined organic phase was washed with brine (2×200 mL), dried over anhydrous Na₂SO₄, filtered, and concentrated under reduced pressure. Purification by column chromatography gave N-((6-hydroxy-3-methyl-1H-indol-2-yl)methyl)-1-methylcyclopropane-1-carboxamide. LC-MS (ESI) m/z calcd for C₁₅H₁₈N₂O₂: 258.14; found: 259.2 [M+H]⁺.

### Step 4: N-((6-(isoxazol-3-ylmethoxy)-3-methyl-1H-indol-2-yl)methyl)-1-methylcyclopropane-1-carboxamide

To a solution of N-((6-hydroxy-3-methyl-1H-indol-2-yl)methyl)-1-methylcyclopropane-1-carboxamide (530 mg, 2.05 mmol, 1.00 eq) and 3-(chloromethyl)isoxazole (41.3 μL, 2.46 mmol, 1.20 eq) in acetone (0.5 mL) were added KI (23.8 mg, 143 μmol, 0.07 eq) and Cs₂CO₃ (668 mg, 2.05 mmol, 1.00 eq) under N₂, and the resulting mixture was heated to 70° C. and stirred for 12 h. The reaction mixture was allowed to cool to room temperature, then was quenched by addition of H₂O (10 mL) and extracted with EtOAc (3×15 mL). The combined organic layers were dried over Na₂SO₄, filtered, and concentrated under reduced pressure. Purification by prep-HPLC gave N-((6-(isoxazol-3-ylmethoxy)-3-methyl-1H-indol-2-yl)methyl)-1-methylcyclopropane-1-carboxamide (Compound 155). LC-MS (ESI) m/z calcd for C₁₉H₂₁N₃O₃: 339.16; found: 340.3 [M+H]⁺. ¹H NMR (400 MHz, DMSO-d₆) δ 8.76 (s, 1H), 8.40 (s, 1H), 7.38 (d, J=8.0 Hz, 1H), 6.88 (s, 1H), 6.82 (d, J=8.0 Hz, 1H), 6.51 (s, 1H), 6.25 (t, J=5.6 Hz, 1H), 5.23 (s, 2H), 4.46 (d, J=6.0 Hz, 2H), 2.27 (s, 3H), 1.31 (s, 3H), 1.27-1.25 (m, 2H), 0.63-0.61 (m, 2H).

The following compounds in Table T-15 were synthesized using procedures similar to Compound 155 using the appropriate starting materials.

TABLE T-15

| | | | Exact | LCMS, |
|---|---|---|---|---|
| Compound | | | mass | found |
| No. | Structure | IUPAC name | (g/mol) | [M + H]⁺ |
| 156 | 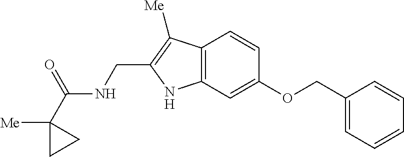 [SMILES: Cc1c(CNC(=O)C2(C)CC2)[nH]c2cc(OCc3ccccc3)ccc12] | N-((6-(benzyloxy)- 3-methyl-1H-indol- 2-yl)methyl)-1- methylcyclopropane- 1-carboxamide | 348.18 | 349.1 |

### Example 28: (Compound 157)

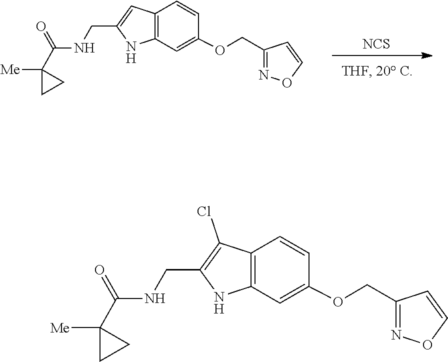 [SMILES: CC1(C(=O)NCc2cc3ccc(OCc4ccon4)cc3[nH]2)CC1 ; "NCS" ; CC1(C(=O)NCc2[nH]c3cc(OCc4ccon4)ccc3c2Cl)CC1]

### Step 1: N-((3-chloro-6-(isoxazol-3-ylmethoxy)-1H-indol-2-yl)methyl)-1-methylcyclopropane-1-carboxamide

To a solution of N-((6-(isoxazol-3-ylmethoxy)-1H-indol-2-yl)methyl)-1-methylcyclopropane-1-carboxamide (190 mg, 584 μmol, 1.00 eq) in THF (4 mL) was added NCS (78.0 mg, 584 μmol, 1.00 eq). The mixture was stirred for 10 min, then was quenched by addition of saturated aqueous NaHSO₃ (10 mL) and extracted with EtOAc (3×15 mL). The organic layer was dried over Na₂SO₄, filtered, and concentrated in vacuo. Purification by prep-HPLC gave N-((3-chloro-6-(isoxazol-3-ylmethoxy)-1H-indol-2-yl)methyl)-1-methylcyclopropane-1-carboxamide (Compound 157). LC-MS (ESI) m/z calcd for C₁₈H₁₈ClN₃O₃: 359.10; found 360.0 [M+H]⁺. ¹H NMR (400 MHz, DMSO-d₆) δ 10.88 (s, 1H), 8.94 (s, 1H), 7.99 (t, J=5.6 Hz, 1H), 7.30 (d, J=8.0 Hz, 1H), 7.06 (s, 1H), 6.83-6.79 (m, 1H), 6.69 (s, 1H), 5.23 (s, 2H), 4.39 (d, J=5.6 Hz, 2H), 1.28 (s, 3H), 1.02-0.99 (m, 2H), 0.54-0.52 (m, 2H).

The following compounds in Table T-16 were synthesized using procedures similar to Compound 157 using the appropriate starting materials.

TABLE T-16

| | | | Exact | LCMS, |
|---|---|---|---|---|
| Compound | | | mass | found |
| No. | Structure | IUPAC name | (g/mol) | [M + H]⁺ |
| 158 | 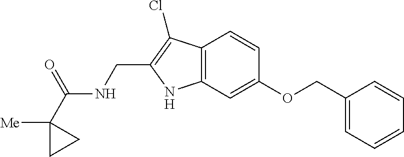 [SMILES: CC1(C(=O)NCc2[nH]c3cc(OCc4ccccc4)ccc3c2Cl)CC1] | N-((6-(benzyloxy)- 3-chloro-1H-indol-2- yl)methyl)-1- methylcyclopropane- 1-carboxamide | 368.13 | 369.2 |

### Example 29: (Compound 159)

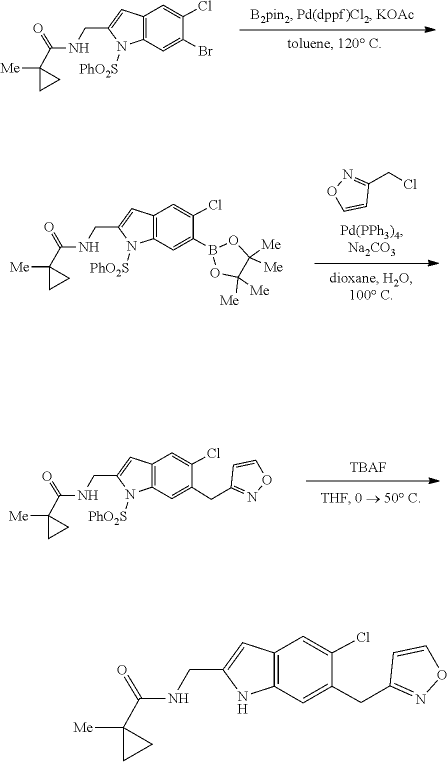

### Step 1: N-((5-chloro-1-(phenylsulfonyl)-6-(4,4,5,5-tetramethyl-1,3,2-dioxaborolan-2-yl)-1H-indol-2-yl)methyl)-1-methylcyclopropanecarboxamide

To a mixture of N-((6-bromo-5-chloro-1-(phenylsulfonyl)-1H-indol-2-yl)methyl)-1-methylcyclopropanecarboxamide (850 mg, 1.76 mmol, 1.00 eq), bis(pinacolato)diboron (672 mg, 2.65 mmol, 1.50 eq) and KOAc (433 mg, 4.41 mmol, 2.50 eq) in toluene (8 mL) under N₂ was added Pd(dppf)Cl₂ (288 mg, 352 μmol, 0.20 eq). The mixture was heated to 120° C. and stirred for 0.5 h. The mixture was allowed to cool to room temperature, then was poured into water (10 mL) and stirred for 2 min. The aqueous phase was extracted with ethyl acetate (2×10 mL). The combined organic phase was washed with brine (10 mL), dried over anhydrous Na₂SO₄, filtered, and concentrated under reduced pressure. Purification by column chromatography gave N-((5-chloro-1-(phenylsulfonyl)-6-(4,4,5,5-tetramethyl-1,3,2-dioxaborolan-2-yl)-1H-indol-2-yl)methyl)-1-methylcyclopropanecarboxamide. LC-MS (ESI) m/z calcd for C₂₆H₃₀BClN₂O₅S: 528.17; found: 529.3 [M+H]⁺.

### Step 2: N-((5-chloro-6-(isoxazol-3-ylmethyl)-1-(phenylsulfonyl)-1H-indol-2-yl)methyl)-1-methylcyclopropanecarboxamide

To a mixture of N-((5-chloro-1-(phenylsulfonyl)-6-(4,4,5,5-tetramethyl-1,3,2-dioxaborolan-2-yl)-1H-indol-2-yl)methyl)-1-methylcyclopropanecarboxamide (800 mg, 1.51 mmol, 1.00 eq), 3-(chloromethyl)isoxazole (213 mg, 1.82 mmol, 1.20 eq) and Na₂CO₃ (481 mg, 4.54 mmol, 3.00 eq) in 1,4-dioxane (10 mL) and H₂O (2 mL) under N₂ was added Pd(PPh₃)₄ (349 mg, 303 μmol, 0.20 eq). The mixture was heated to 100° C. and stirred for 1 h. The reaction mixture was allowed to cool to room temperature, then was poured into water (10 mL) and stirred for 5 min. The aqueous phase was extracted with ethyl acetate (2×10 mL). The combined organic phase was dried over anhydrous Na₂SO₄, filtered, and concentrated under reduced pressure. Purification by column chromatography gave N-((5-chloro-6-(isoxazol-3-ylmethyl)-1-(phenylsulfonyl)-1H-indol-2-yl)methyl)-1-methylcyclopropanecarboxamide. LC-MS (ESI) m/z calcd for C₂₄H₂₂ClN₃O₄S: 483.10; found: 484.1 [M+H]⁺.

### Step 3: N-((5-chloro-6-(isoxazol-3-ylmethyl)-1H-indol-2-yl)methyl)-1-methylcyclopropanecarboxamide

To a solution of N-((5-chloro-6-(isoxazol-3-ylmethyl)-1-(phenylsulfonyl)-1H-indol-2-yl)methyl)-1-methylcyclopropanecarboxamide (200 mg, 413 μmol, 1.00 eq) in THF (3 mL) at 0° C. was added TBAF (1 M in THF, 4.13 mL, 4.13 mmol, 10.0 eq) dropwise. The mixture was then heated to 50° C. and stirred for 2 h. The reaction mixture was allowed to cool to room temperature, then was poured into water (10 mL) and stirred for 2 min. The aqueous phase was extracted with ethyl acetate (3×10 mL). The combined organic phase was dried over anhydrous Na₂SO₄, filtered, and concentrated under reduced pressure. Purification by prep-HPLC gave N-((5-chloro-6-(isoxazol-3-ylmethyl)-1H-indol-2-yl)methyl)-1-methylcyclopropanecarboxamide (Compound 159). LC-MS (ESI) m/z calcd for C₁₈H₁₈ClN₃O₂: 343.11; found 344.0 [M+H]⁺. ¹H NMR (400 MHz, DMSO-d₆) δ 10.92 (s, 1H), 8.79 (d, J=1.6 Hz, 1H), 8.04 (t, J=5.6 Hz, 1H), 7.53 (s, 1H), 7.30 (s, 1H), 6.33 (s, 1H), 6.17 (s, 1H), 4.37 (d, J=5.6 Hz, 2H), 4.16 (s, 2H), 1.29 (s, 3H), 1.00-0.98 (m, 2H), 0.54-0.51 (m, 2H).

### Example 30: (Compound 160)

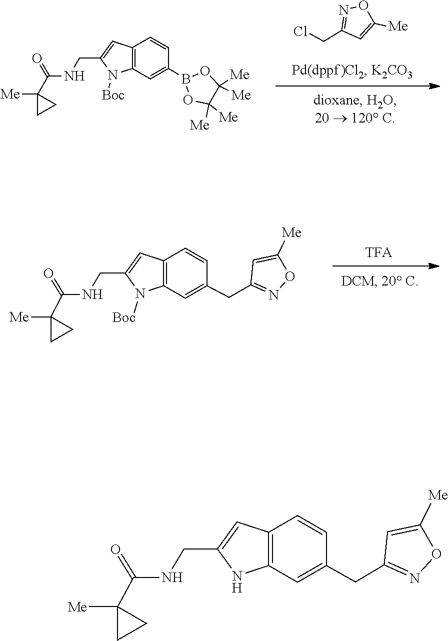 [SMILES: CC(C)(C)OC(=O)n1c(CNC(=O)C2(C)CC2)cc2ccc(B3OC(C)(C)C(C)(C)O3)cc21 ; Cc1cc(CCl)no1 ; Cc1cc(Cc2ccc3cc(CNC(=O)C4(C)CC4)n(C(=O)OC(C)(C)C)c3c2)no1 ; Cc1cc(Cc2ccc3cc(CNC(=O)C4(C)CC4)[nH]c3c2)no1]

### Step 1: tert-butyl 2-((1-methylcyclopropanecarboxamido)methyl)-6-((5-methylisoxazol-3-yl)methyl)-1H-indole-1-carboxylate

To a mixture of tert-butyl 2-((1-methylcyclopropanecarboxamido)methyl)-6-(4,4,5,5-tetramethyl-1,3,2-dioxaborolan-2-yl)-1H-indole-1-carboxylate (200 mg, 440 μmol, 1.00 eq) and 3-(chloromethyl)-5-methylisoxazole (86 mg, 660 μmol, 1.50 eq) in dioxane (1 mL) and H₂O (0.2 mL) were added K₂CO₃ (182 mg, 1.32 mmol, 3.00 eq) and Pd(dppf)Cl₂ (107 mg, 132 μmol, 0.30 eq) under N₂. The mixture was then heated to 120° C. and stirred for 4 h. The reaction mixture was allowed to cool to room temperature, then was poured into water (5 mL). The aqueous phase was extracted with ethyl acetate (2×10 mL). The combined organic phase was washed with brine (10 mL), dried over anhydrous sodium sulfate, filtered, and concentrated under reduced pressure. Purification by column chromatography gave tert-butyl 2-((1-methylcyclopropanecarboxamido)methyl)-6-((5-methylisoxazol-3-yl)methyl)-1H-indole-1-carboxylate. LC-MS (ESI) m/z calcd for C₂₄H₂₉N₃O₄: 423.22; found 424.1 [M+H]⁺.

### Step 2: 1-methyl-N-((6-((5-methylisoxazol-3-yl)methyl)-1H-indol-2-yl)methyl)cyclopropanecarboxamide

To a solution of tert-butyl 2-((1-methylcyclopropanecarboxamido)methyl)-6-((5-methylisoxazol-3-yl)methyl)-1H-indole-1-carboxylate (120 mg, 226 μmol, 1.00 eq) in DCM (3 mL) was added TFA (0.5 mL), and the mixture was stirred for 6 h. The reaction was then concentrated under reduced pressure and purified by prep-HPLC to give 1-methyl-N-((6-((5-methylisoxazol-3-yl)methyl)-1H-indol-2-yl)methyl)cyclopropanecarboxamide (Compound 160). LC-MS (ESI) m/z calcd for C₁₉H₂₁N₃O₂: 323.16; found 324.2 [M+H]⁺. ¹H NMR (400 MHz, DMSO-d₆) δ 10.74 (s, 1H), 7.99 (t, J=5.2 Hz, 1H), 7.35 (d, J=8.0 Hz, 1H), 7.22 (d, J=9.2 Hz, 1H), 6.87-6.84 (m, 1H), 6.17 (s, 1H), 5.94 (s, 1H), 4.39 (d, J=5.6 Hz, 2H), 3.95 (s, 2H), 2.32 (s, 3H), 1.30 (s, 3H), 1.03-1.01 (m, 2H), 0.54-0.51 (m, 2H).

### Example 31: (Compound 161)

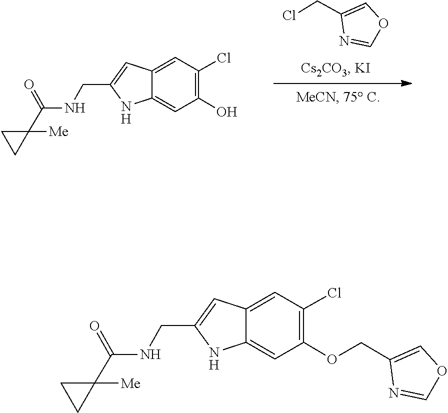 [SMILES: ClCc1cocn1 ; CC1(C(=O)NCc2cc3cc(Cl)c(O)cc3[nH]2)CC1 ; CC1(C(=O)NCc2cc3cc(Cl)c(OCc4cocn4)cc3[nH]2)CC1]

### Step 1: N-((5-chloro-6-(oxazol-4-ylmethoxy)-1H-indol-2-yl)methyl)-1-methylcyclopropane-1-carboxamide

To a mixture of 4-(chloromethyl)oxazole (23.2 mg, 197 μmol, 1.10 eq) and N-((5-chloro-6-hydroxy-1H-indol-2-yl)methyl)-1-methylcyclopropane-1-carboxamide (50.0 mg, 179 μmol, 1.00 eq) in MeCN (1 mL) were added KI (1.5 mg, 9 μmol, 0.05 eq) and Cs₂CO₃ (64.3 mg, 197 μmol, 1.10 eq). The reaction mixture was heated to 75° C. and stirred for 18 h. The mixture was allowed to cool to room temperature, was quenched by addition of water (5 mL), and was extracted with ethyl acetate (3×5 mL). The combined organic layers were washed with brine (10 mL), dried over Na₂SO₄, filtered, and concentrated under reduced pressure. Purification by prep-HPLC gave N-((5-chloro-6-(oxazol-4-ylmethoxy)-1H-indol-2-yl)methyl)-1-methylcyclopropane-1-carboxamide (Compound 161). LC-MS (ESI) m/z calcd for C₁₈H₁₈ClN₃O₃: 359.10; found 360.2 [M+H]⁺. ¹H NMR (500 MHz, CD₃CN) δ 9.29 (s, 1H) 7.93 (s, 1H), 7.83-7.81 (m, 1H), 7.40 (s, 1H), 7.09 (s, 1H), 6.12-6.09 (m, 1H), 4.96 (s, 2H), 4.32-4.30 (m, 2H), 1.19 (s, 3H), 0.98 (q, J=3.6 Hz, 2H), 0.49 (q, J=3.6 Hz, 2H).

The following compounds in Table T-17 were synthesized using procedures similar to Compound 161 using the appropriate starting materials.

TABLE T-17

| | | | Exact | LCMS, |
|---|---|---|---|---|
| Compound | | | mass | found |
| No. | Structure | IUPAC name | (g/mol) | [M + H]⁺ |
| 162 | 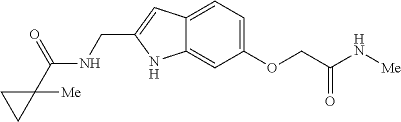 [SMILES: CNC(=O)COc1ccc2cc(CNC(=O)C3(C)CC3)[nH]c2c1] | 1-methyl-N-((6-(2- (methylamino)-2- oxoethoxy)-1H-indol-2- yl)methyl)cyclopropane- 1-carboxamide | 315.2 | 316.1 |
| 163 | 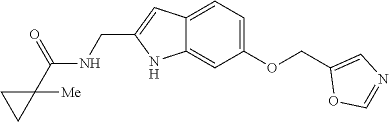 [SMILES: CC1(C(=O)NCc2cc3ccc(OCc4cnco4)cc3[nH]2)CC1] | 1-methyl-N-((6-(oxazol- 5-ylmethoxy)-1H-indol- 2- yl)methyl)cyclopropane- 1-carboxamide | 325.1 | 326.1 |
| 164 | 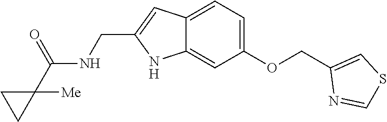 [SMILES: CC1(C(=O)NCc2cc3ccc(OCc4cscn4)cc3[nH]2)CC1] | 1-methyl-N-((6-(thiazol- 4-ylmethoxy)-1H-indol- 2- yl)methyl)cyclopropane- 1-carboxamide | 341.1 | 341.9 |
| 165 | 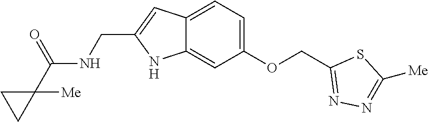 [SMILES: Cc1nnc(COc2ccc3cc(CNC(=O)C4(C)CC4)[nH]c3c2)s1] | 1-methyl-N-((6-((5- methyl-1,3,4-thiadiazol- 2-yl)methoxy)-1H- indol-2- yl)methyl)cyclopropane- 1-carboxamide | 356.1 | 357.5 |
| 166 | 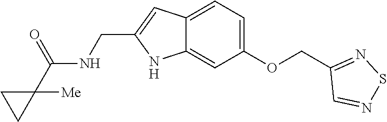 [SMILES: CC1(C(=O)NCc2cc3ccc(OCc4cnsn4)cc3[nH]2)CC1] | N-((6-((1,2,5-thiadiazol- 3-yl)methoxy)-1H- indol-2-yl)methyl)-1- methylcyclopropane-1- carboxamide | 342.1 | 343.4 |
| 167 | 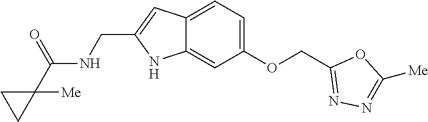 [SMILES: Cc1nnc(COc2ccc3cc(CNC(=O)C4(C)CC4)[nH]c3c2)o1] | 1-methyl-N-((6-((5- methyl-1,3,4-oxadiazol- 2-yl)methoxy)-1H- indol-2- yl)methyl)cyclopropane- 1-carboxamide | 340.2 | 341.2 |
| 168 | 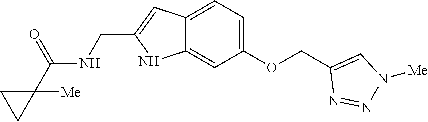 [SMILES: Cn1cc(COc2ccc3cc(CNC(=O)C4(C)CC4)[nH]c3c2)nn1] | 1-methyl-N-((6-((1- methyl-1H-1,2,3-triazol- 4-yl)methoxy)-1H- indol-2- yl)methyl)cyclopropane- 1-carboxamide | 339.2 | 339.8 |
| 169 | 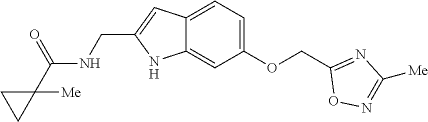 [SMILES: Cc1noc(COc2ccc3cc(CNC(=O)C4(C)CC4)[nH]c3c2)n1] | 1-methyl-N-((6-((3- methyl-1,2,4-oxadiazol- 5-yl)methoxy)-1H- indol-2- yl)methyl)cyclopropane- 1-carboxamide | 340.2 | 341.1 |
| 170 | 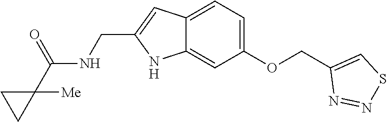 [SMILES: CC1(C(=O)NCc2cc3ccc(OCc4csnn4)cc3[nH]2)CC1] | N-((6-((1,2,3-thiadiazol- 4-yl)methoxy)-1H- indol-2-yl)methyl)-1- methylcyclopropane-1- carboxamide | 342.1 | 342.8 |
| 171 | 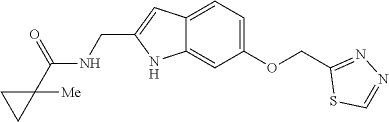 [SMILES: CC1(C(=O)NCc2cc3ccc(OCc4nncs4)cc3[nH]2)CC1] | N-((6-((1,3,4-thiadiazol- 2-yl)methoxy)-1H- indol-2-yl)methyl)-1- methylcyclopropane-1- carboxamide | 342.1 | 342.6 |
| 172 | 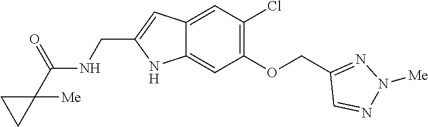 [SMILES: Cn1ncc(COc2cc3[nH]c(CNC(=O)C4(C)CC4)cc3cc2Cl)n1] | N-((5-chloro-6-((2- methyl-2H-1,2,3-triazol- 4-yl)methoxy)-1H- indol-2-yl)methyl)-1- methylcyclopropane-1- carboxamide | 373.1 | 374.3 |
| 173 | 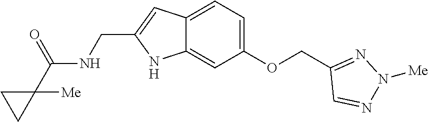 [SMILES: Cn1ncc(COc2ccc3cc(CNC(=O)C4(C)CC4)[nH]c3c2)n1] | 1-methyl-N-((6-((2- methyl-2H-1,2,3-triazol- 4-yl)methoxy)-1H- indol-2- yl)methyl)cyclopropane- 1-carboxamide | 339.2 | 340.2 |
| 174 | 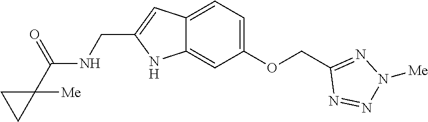 [SMILES: Cn1nnc(COc2ccc3cc(CNC(=O)C4(C)CC4)[nH]c3c2)n1] | 1-methyl-N-((6-((2- methyl-2H-tetrazol-5- yl)methoxy)-1H-indol- 2- yl)methyl)cyclopropane- 1-carboxamide | 340.2 | 341.2 |
| 175 | 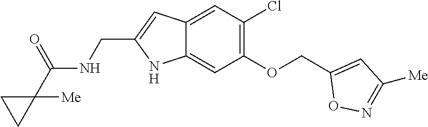 [SMILES: Cc1cc(COc2cc3[nH]c(CNC(=O)C4(C)CC4)cc3cc2Cl)on1] | N-((5-chloro-6-((3- methylisoxazol-5- yl)methoxy)-1H-indol- 2-yl)methyl)-1- methylcyclopropane-1- carboxamide | 373.1 | 374.2 |
| 176 | 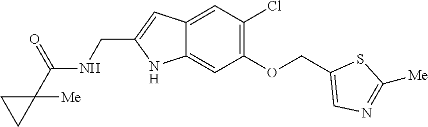 [SMILES: Cc1ncc(COc2cc3[nH]c(CNC(=O)C4(C)CC4)cc3cc2Cl)s1] | N-((5-chloro-6-((2- methylthiazol-5- yl)methoxy)-1H-indol- 2-yl)methyl)-1- methylcyclopropane-1- carboxamide | 389.1 | 390.4 |
| 177 | 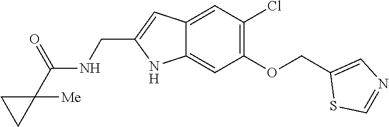 [SMILES: CC1(C(=O)NCc2cc3cc(Cl)c(OCc4cncs4)cc3[nH]2)CC1] | N-((5-chloro-6-(thiazol- 5-ylmethoxy)-1H-indol- 2-yl)methyl)-1- methylcyclopropane-1- carboxamide | 375.1 | 376.2 |
| 178 | 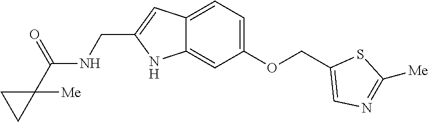 [SMILES: Cc1ncc(COc2ccc3cc(CNC(=O)C4(C)CC4)[nH]c3c2)s1] | 1-methyl-N-((6-((2- methylthiazol-5- yl)methoxy)-1H-indol- 2- yl)methyl)cyclopropane- 1-carboxamide | 355.1 | 355.8 |
| 179 | 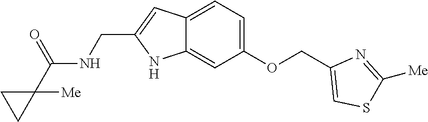 [SMILES: Cc1nc(COc2ccc3cc(CNC(=O)C4(C)CC4)[nH]c3c2)cs1] | 1-methyl-N-((6-((2- methylthiazol-4- yl)methoxy)-1H-indol- 2- yl)methyl)cyclopropane- 1-carboxamide | 355.1 | 356.5 |
| 180 | 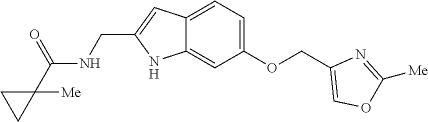 [SMILES: Cc1nc(COc2ccc3cc(CNC(=O)C4(C)CC4)[nH]c3c2)co1] | 1-methyl-N-((6-((2- methyloxazol-4- yl)methoxy)-1H-indol- 2- yl)methyl)cyclopropane- 1-carboxamide | 339.2 | 339.7 |
| 181 | 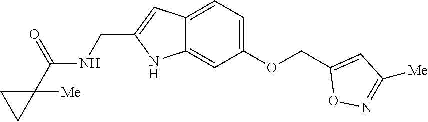 [SMILES: Cc1cc(COc2ccc3cc(CNC(=O)C4(C)CC4)[nH]c3c2)on1] | 1-methyl-N-((6-((3- methylisoxazol-5- yl)methoxy)-1H-indol- 2- yl)methyl)cyclopropane- 1-carboxamide | 339.2 | 339.7 |
| 182 | 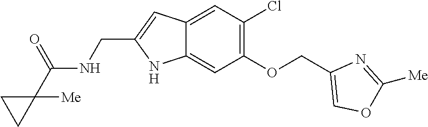 [SMILES: Cc1nc(COc2cc3[nH]c(CNC(=O)C4(C)CC4)cc3cc2Cl)co1] | N-((5-chloro-6-((2- methyloxazol-4- yl)methoxy)-1H-indol- 2-yl)methyl)-1- methylcyclopropane-1- carboxamide | 373.12 | 374.2 |
| 183 | 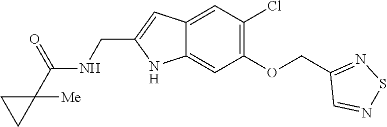 [SMILES: CC1(C(=O)NCc2cc3cc(Cl)c(OCc4cnsn4)cc3[nH]2)CC1] | N-((6-((1,2,5-thiadiazol- 3-yl)methoxy)-5-chloro- 1H-indol-2-yl)methyl)- 1-methylcyclopropane- 1-carboxamide | 376.1 | 377.1 |
| 184 | 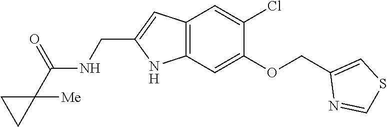 [SMILES: CC1(C(=O)NCc2cc3cc(Cl)c(OCc4cscn4)cc3[nH]2)CC1] | N-((5-chloro-6-(thiazol- 4-ylmethoxy)-1H-indol- 2-yl)methyl)-1- methylcyclopropane-1- carboxamide | 375.1 | 376.1 |
| 185 | 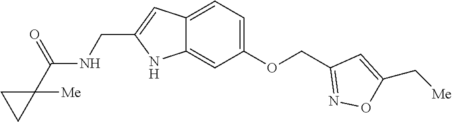 [SMILES: CCc1cc(COc2ccc3cc(CNC(=O)C4(C)CC4)[nH]c3c2)no1] | N-((6-((5-ethylisoxazol- 3-yl)methoxy)-1H- indol-2-yl)methyl)-1- methylcyclopropane-1- carboxamide | 353.2 | 354.2 |
| 186 | 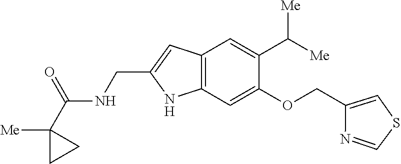 [SMILES: CC(C)c1cc2cc(CNC(=O)C3(C)CC3)[nH]c2cc1OCc1cscn1] | N-((5-isopropyl-6- (thiazol-4-ylmethoxy)- 1H-indol-2-yl)methyl)- 1-methylcyclopropane- 1-carboxamide | 383.17 | 384.2 |
| 204 | 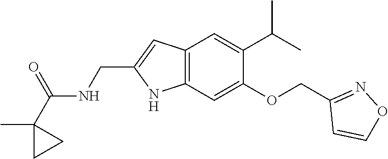 [SMILES: CC(C)c1cc2cc(CNC(=O)C3(C)CC3)[nH]c2cc1OCc1ccon1] | N-((5-isopropyl-6- (isoxazol-3-ylmethoxy)- 1H-indol-2-yl)methyl)- 1-methylcyclopropane- 1-carboxamide | 367.19 | 368.2 |

### Example 32: (Compound 187)

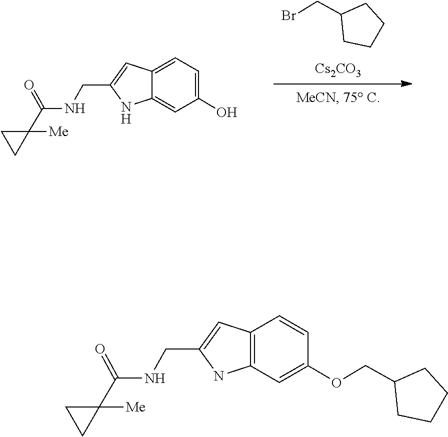

### Step 1: N-((6-(cyclopentylmethoxy)-1H-indol-2-yl)methyl)-1-methylcyclopropane-1-carboxamide

To a mixture of (bromomethyl)cyclopentane (30.4 mg, 186 μmol, 1.00 eq) and N-((6-hydroxy-1H-indol-2-yl)methyl)-1-methylcyclopropane-1-carboxamide (50.0 mg, 205 μmol, 1.10 eq) in MeCN (1 mL) was added Cs₂CO₃ (121 mg, 372 μmol, 2.00 eq). The reaction mixture was heated to 75° C. and stirred for 18 h. The mixture was allowed to cool to room temperature, was quenched by addition of water (5 mL), and was extracted with ethyl acetate (3×5 mL). The combined organic layers were washed with brine (10 mL), dried over Na₂SO₄, filtered, and concentrated under reduced pressure. Purification by prep-HPLC gave N-((6-(cyclopentylmethoxy)-1H-indol-2-yl)methyl)-1-methylcyclopropane-1-carboxamide (Compound 187). LC-MS (ESI) m/z calcd for C₂₀H₂₆N₂O₂: 326.20; found: 327.3 [M+H]⁺. ¹H NMR (500 MHz, MeOD) δ 7.20 (d, J=8.6 Hz, 1H), 6.75 (d, J=2.2 Hz, 1H), 6.53 (dd, J=8.6, 2.2 Hz, 1H), 6.09 (s, 1H), 4.36 (s, 2H), 3.74 (d, J=6.9 Hz, 2H), 2.26 (hept, J=7.4 Hz, 1H), 1.80-1.45 (m, 6H), 1.36-1.26 (m, 2H), 1.24 (s, 3H), 1.04 (q, J=3.8 Hz, 2H), 0.52 (q, J=3.8 Hz, 2H).

The following compounds in Table T-18 were synthesized using procedures similar to Compound 187 using the appropriate starting materials.

TABLE T-18

| | | | Exact | LCMS, |
|---|---|---|---|---|
| Compound | | | mass | found |
| No. | Structure | IUPAC name | (g/mol) | [M + H]⁺ |
| 188 | 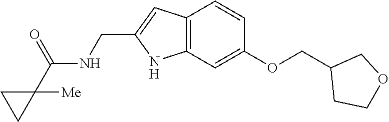 [SMILES: CC1(C(=O)NCc2cc3ccc(OCC4CCOC4)cc3[nH]2)CC1] | 1-methyl-N-((6- ((tetrahydrofuran-3- yl)methoxy)-1H-indol- 2- yl)methyl)cyclopropane- 1-carboxamide | 328.2 | 329.2 |
| 189 | 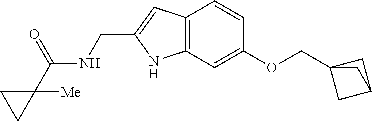 [SMILES: CC1(C(=O)NCc2cc3ccc(OCC45CC(C4)C5)cc3[nH]2)CC1] | N-((6- (bicyclo[1.1.1]pentan-1- ylmethoxy)-1H-indol-2- yl)methyl)-1- methylcyclopropane-1- carboxamide | 324.2 | 325.3 |

### Example 33: (Compound 190)

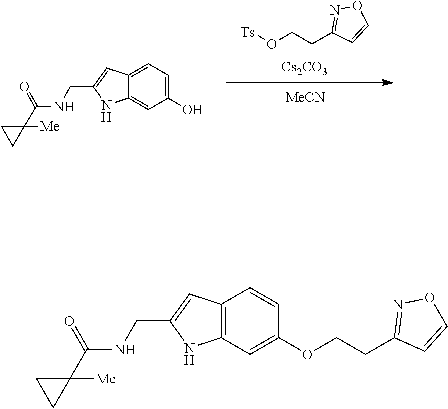 [SMILES: "MeCN" ; Cc1ccc(S(=O)(=O)OCCc2ccon2)cc1 ; CC1(C(=O)NCc2cc3ccc(O)cc3[nH]2)CC1 ; "Cs2CO3" ; CC1(C(=O)NCc2cc3ccc(OCCc4ccon4)cc3[nH]2)CC1]

### Step 1. N-((6-(2-(isoxazol-3-yl)ethoxy)-1H-indol-2-yl)methyl)-1-methylcyclopropane-1-carboxamide

A solution of N-((6-hydroxy-1H-indol-2-yl)methyl)-1-methylcyclopropane-1-carboxamide (119 mg, 488 μmol, 1.00 eq) in MeCN (1 mL) was added Cs₂CO₃ (238 mg, 732 μmol, 1.50 eq).

After 5 min, 2-(isoxazol-3-yl)ethyl 4-methylbenzenesulfonate (130 mg, 488 μmol, 1.00 eq) was added. The resulting mixture stirred at room temperature for 18 h. The reaction mixture was concentrated under reduced pressure and purified by prep-HPLC to give N-((6-(2-(isoxazol-3-yl)ethoxy)-1H-indol-2-yl)methyl)-1-methylcyclopropane-1-carboxamide (Compound 190). LC-MS (ESI) m/z calcd for C₁₉H₂₁N₃O₃: 339.16; found: 339.8 [M+H]. ¹H NMR (500 MHz, CD₃CN) 6 9.14 (s, 1H), 8.40 (d, J=1.6 Hz, 1H), 7.25 (d, J=8.5 Hz, 1H), 6.87 (s, 1H), 6.83 (d, J=2.3 Hz, 1H), 6.56 (dd, J=8.6, 2.3 Hz, 1H), 6.37 (d, J=1.6 Hz, 1H), 6.12-6.10 (m, 1H), 4.30 (d, J=5.9 Hz, 2H), 4.18 (t, J=6.4 Hz, 2H), 3.06 (t, J=6.4 Hz, 2H), 1.19 (s, 3H), 0.98 (q, J=3.6 Hz, 2H), 0.48 (q, J=3.6 Hz, 2H).

The following compounds in Table T-18.1 were synthesized using procedures similar to Compound 190 using the appropriate starting materials.

TABLE T-18.1

| | | | Exact | LCMS, |
|---|---|---|---|---|
| Compound | | | mass | found |
| No. | Structure | IUPAC name | (g/mol) | [M + H]⁺ |
| 454 | 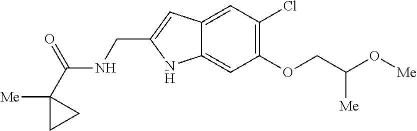 [SMILES: COC(C)COc1cc2[nH]c(CNC(=O)C3(C)CC3)cc2cc1Cl] | N-((5-chloro-6-(2- methoxypropoxy)-1H-indol-2- yl)methyl)-1- methylcyclopropane-1- carboxamide | 350.14 | |
| 455 | 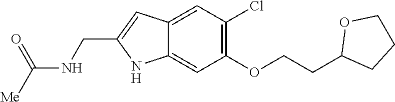 [SMILES: CC(=O)NCc1cc2cc(Cl)c(OCCC3CCCO3)cc2[nH]1] | N-((5-chloro-6-(2- (tetrahydrofuran-2-yl)ethoxy)- 1H-indol-2- yl)methyl)acetamide | 336.12 | 337.2 |
| 456 | 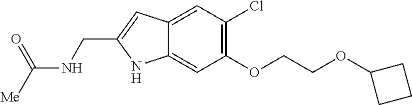 [SMILES: CC(=O)NCc1cc2cc(Cl)c(OCCOC3CCC3)cc2[nH]1] | N-((5-chloro-6-(2- cyclobutoxyethoxy)-1H-indol- 2-yl)methyl)acetamide | 336.12 | 337.2 |
| 457 | 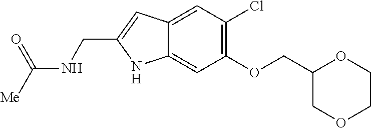 [SMILES: CC(=O)NCc1cc2cc(Cl)c(OCC3COCCO3)cc2[nH]1] | N-((6-((1,4-dioxan-2- yl)methoxy)-5-chloro-1H- indol-2-yl)methyl)acetamide | 338.10 | 339.6 |
| 458 | 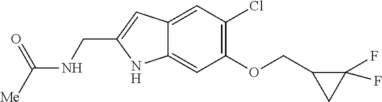 [SMILES: CC(=O)NCc1cc2cc(Cl)c(OCC3CC3(F)F)cc2[nH]1] | N-((5-chloro-6-((2,2- difluorocyclopropyl)methoxy)- 1H-indol-2- yl)methyl)acetamide | 328.08 | 329.1 |
| 459 | 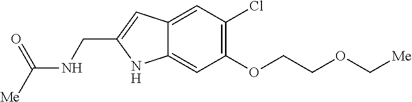 [SMILES: CCOCCOc1cc2[nH]c(CNC(C)=O)cc2cc1Cl] | N-((5-chloro-6-(2- ethoxyethoxy)-1H-indol-2- yl)methyl)acetamide | 310.11 | 311.2 |

### Example 34: (Compound 191)

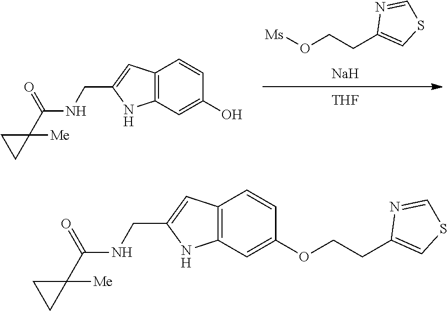

### 1-methyl-N-((6-(2-(thiazol-4-yl)ethoxy)-1H-indol-2-yl)methyl)cyclopropane-1-carboxamide

To a solution of N-((6-hydroxy-1H-indol-2-yl)methyl)-1-methylcyclopropane-1-carboxamide (239 mg, 980 μmol, 1.50 eq) in THF (2 mL) at 0° C. was added NaH (39.2 mg, 60 w/w %, 980 μmol, 1.50 eq). After 15 min, 2-(thiazol-4-yl)ethyl methanesulfonate (135 mg, 653 μmol, 1.00 eq) was added. After 16 h the reaction was quenched by addition of MeOH. The mixture was filtered, concentrated under reduced pressure, and purified by prep-HPLC to give 1-methyl-N-((6-(2-(thiazol-4-yl)ethoxy)-1H-indol-2-yl)methyl)cyclopropane-1-carboxamide (Compound 191). LC-MS (ESI) m/z calcd for C₁₉H₂₁N₃O₂S: 355.14; found 355.9 [M+H]⁺. ¹H NMR (500 MHz, CD₃CN) 6 9.10 (s, 1H), 8.64 (s, 1H), 7.63 (s, 1H), 7.26 (d, J=8.6 Hz, 1H), 6.83 (d, J=2.3 Hz, 1H), 6.59 (dd, J=8.6, 2.3 Hz, 1H), 6.13-6.09 (m, 1H), 4.30 (d, J=5.8 Hz, 2H), 4.11 (t, J=6.1 Hz, 2H), 3.27-3.21 (m, 2H), 1.19 (s, 3H), 0.98 (q, J=3.5 Hz, 2H), 0.48 (q, J=3.6 Hz, 2H).

### Example 35: (Compound 192)

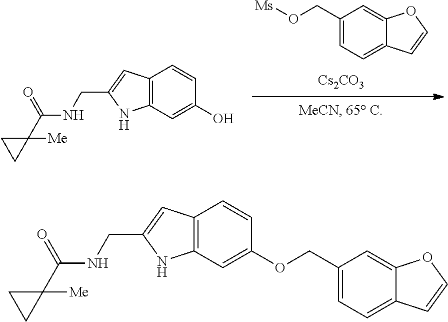 [SMILES: CS(=O)(=O)OCc1ccc2ccoc2c1 ; CC1(C(=O)NCc2cc3ccc(O)cc3[nH]2)CC1 ; "Cs2CO3" ; "MeCN, 65° C." ; CC1(C(=O)NCc2cc3ccc(OCc4ccc5ccoc5c4)cc3[nH]2)CC1]

### N-((6-(benzofuran-6-ylmethoxy)-1H-indol-2-yl)methyl)-1-methylcyclopropane-1-carboxamide

To a mixture of benzofuran-6-ylmethyl methanesulfonate (315 mg, 1.39 mmol, 1.00 eq) and N-((6-hydroxy-1H-indol-2-yl)methyl)-1-methylcyclopropane-1-carboxamide (341 mg, 1.39 mmol, 1.00 eq) in MeCN (3 mL) was added Cs₂CO₃ (681 mg, 2.09 mmol, 1.50 eq). The reaction mixture was heated to 65° C. and stirred for 18 h. The mixture was allowed to cool to room temperature, then was quenched with water (5 mL) and extracted with ethyl acetate (3×5 mL). The combined organic layers were washed with brine (10 mL), dried over Na₂SO₄, filtered, and concentrated under reduced pressure. Purification by prep-HPLC gave N-((6-(benzofuran-6-ylmethoxy)-1H-indol-2-yl)methyl)-1-methylcyclopropane-1-carboxamide (Compound 192). LC-MS (ESI) m/z calcd for C₂₃H₂₂N₂O₃: 374.16; found 374.9 [M+H]⁺. ¹H NMR (500 MHz, CD₃CN) δ 9.21 (s, 1H), 7.78 (d, J=2.2 Hz, 1H), 7.70-7.65 (m, 2H), 7.39 (d, J=8.6 Hz, 2H), 7.02 (s, 1H), 6.95 (s, 1H), 6.89 (s, 1H), 6.81-6.75 (m, 1H), 6.25-6.21 (m, 1H), 5.23 (s, 2H), 4.44-4.37 (m, 2H), 1.30 (s, 3H), 1.09 (q, J=4.6 Hz, 2H), 0.60 (q, J=3.8 Hz, 2H).

The following compounds in Table T-19 were synthesized using procedures similar to Compound 192 using the appropriate starting materials.

TABLE T-19

| | | | Exact | LCMS, |
|---|---|---|---|---|
| Compound | | | mass | found |
| No. | Structure | IUPAC name | (g/mol) | [M + H]⁺ |
| 193 | 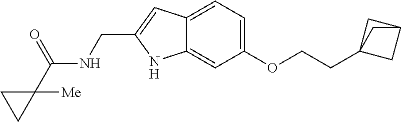 [SMILES: CC1(C(=O)NCc2cc3ccc(OCCC45CC(C4)C5)cc3[nH]2)CC1] | N-((6-(2- (bicyclo[1.1.1]pentan- 1-yl)ethoxy)-1H- indol-2-yl)methyl)-1- methylcyclopropane- 1-carboxamide | 338.2 | 339.6 |
| 460 | 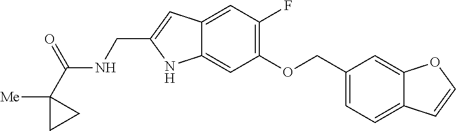 [SMILES: CC1(C(=O)NCc2cc3cc(F)c(OCc4ccc5ccoc5c4)cc3[nH]2)CC1] | N-((6-(benzofuran-6- ylmethoxy)-5-fluoro- 1H-indol-2- yl)methyl)-1- methylcyclopropane- 1-carboxamide | 392.15 | 393.0 |
| 461 | 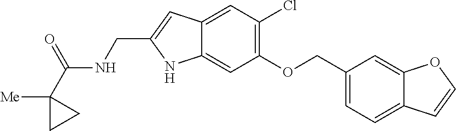 [SMILES: CC1(C(=O)NCc2cc3cc(Cl)c(OCc4ccc5ccoc5c4)cc3[nH]2)CC1] | N-((6-(benzofuran-6- ylmethoxy)-5-chloro- 1H-indol-2- yl)methyl)-1- methylcyclopropane- 1-carboxamide | 408.12 | 409.2 |
| 462 | 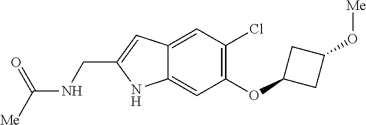 [SMILES: CO[C@H]1C[C@H](Oc2cc3[nH]c(CNC(C)=O)cc3cc2Cl)C1] | N-((5-chloro-6- ((trans)-3- methoxycyclobutoxy)- 1H-indol-2- yl)methyl)acetamide | 322.11 | 323.1 |
| 463 | 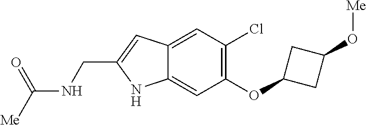 [SMILES: CO[C@H]1C[C@@H](Oc2cc3[nH]c(CNC(C)=O)cc3cc2Cl)C1] | N-((5-chloro-6-((cis)- 3- methoxycyclobutoxy)- 1H-indol-2- yl)methyl)acetamide | 322.11 | 323.2 |
| 464 | 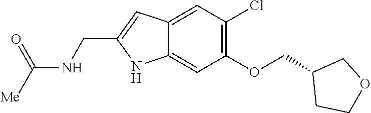 [SMILES: CC(=O)NCc1cc2cc(Cl)c(OC[C@H]3CCOC3)cc2[nH]1] | (S)-N-((5-chloro-6- ((tetrahydrofuran-3- yl)methoxy)-1H-indol- 2-yl)methyl)acetamide | 322.11 | 323.0 |
| 465 | 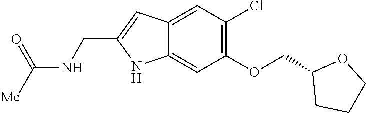 [SMILES: CC(=O)NCc1cc2cc(Cl)c(OC[C@H]3CCCO3)cc2[nH]1] | (R)-N-((5-chloro-6- ((tetrahydrofuran-2- yl)methoxy)-1H-indol- 2-yl)methyl)acetamide | 322.11 | 323.2 |
| 466 | 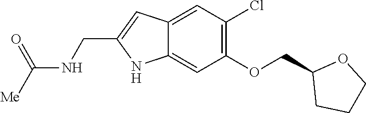 [SMILES: CC(=O)NCc1cc2cc(Cl)c(OC[C@@H]3CCCO3)cc2[nH]1] | (S)-N-((5-chloro-6- ((tetrahydrofuran-2- yl)methoxy)-1H-indol- 2-yl)methyl)acetamide | 322.11 | 323.2 |

### Example 36: (Compound 194)

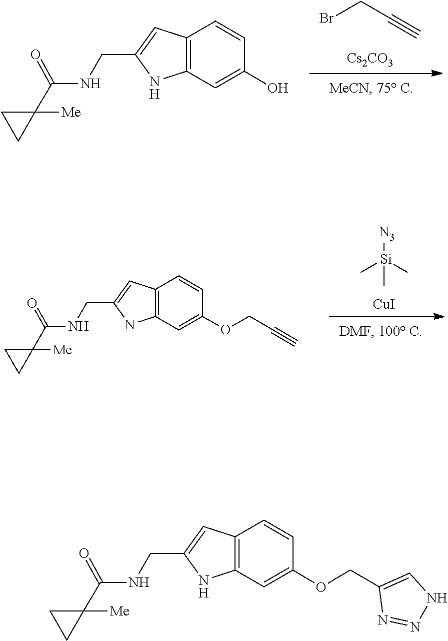

### Step 1: 1-methyl-N-((6-(prop-2-yn-1-yloxy)-1H-indol-2-yl)methyl)cyclopropane-1-carboxamide

To a solution of N-((6-hydroxy-1H-indol-2-yl)methyl)-1-methylcyclopropane-1-carboxamide (500 mg, 2.05 mmol, 1 eq) in MeCN (4 mL) were added Cs₂CO₃ (800 mg, 2.46 mmol, 1.20 eq) and 3-bromoprop-1-yne (268 mg, 335 μL, 80 w/v %, 2.25 mmol, 1.1 eq). The reaction mixture was stirred for 18 h at room temperature. The reaction mixture was quenched with water (10 mL) and extracted with ethyl acetate (3×10 mL). The combined organics were washed with brine (15 mL), dried over Na₂SO₄, filtered, and concentrated under reduced pressure to give 1-methyl-N-((6-(prop-2-yn-1-yloxy)-1H-indol-2-yl)methyl)cyclopropane-1-carboxamide, which was used without further purification. LC-MS (ESI) m/z calcd for C₁₇H₁₈N₂O₂: 282.1; found 283.2 [M+H]⁺.

### Step 2: N-((6-((1H-1,2,3-triazol-4-yl)methoxy)-1H-indol-2-yl)methyl)-1-methylcyclopropane-1-carboxamide

To a mixture of 1-methyl-N-((6-(prop-2-yn-1-yloxy)-1H-indol-2-yl)methyl)cyclopropane-1-carboxamide (578 mg, 2.05 mmol, 1 eq) and azidotrimethylsilane (354 mg, 404 μL, 3.07 mmol, 1.5 eq) in DMF (4 mL) was added CuI (19.5 mg, 102 μmol, 0.05 eq) under N₂. The reaction mixture was heated to 100° C. and stirred for 18 h. The mixture was allowed to cool to room temperature, then was filtered. Purification by prep-HPLC gave N-((6-((1H-1,2,3-triazol-4-yl)methoxy)-1H-indol-2-yl)methyl)-1-methylcyclopropane-1-carboxamide (Compound 194). LC-MS (ESI) m/z calcd for C₁₇H₁₉N₅O₂: 325.2; found 325.7 [M+H]⁺. ¹H NMR (500 MHz, CD₃CN) δ 9.25 (s, 1H), 7.79 (s, 1H), 7.36 (d, J=8.6 Hz, 1H), 7.01 (d, J=2.3 Hz, 1H), 6.96 (s, 1H), 6.72 (dd, J=8.6, 2.3 Hz, 1H), 6.23-6.19 (m, 1H), 5.18 (s, 2H), 4.40 (d, J=5.9 Hz, 2H), 1.28 (s, 3H), 1.07 (q, J=3.6 Hz, 2H), 0.58 (q, J=3.6 Hz, 2H).

### Example 37: (Compound 195)

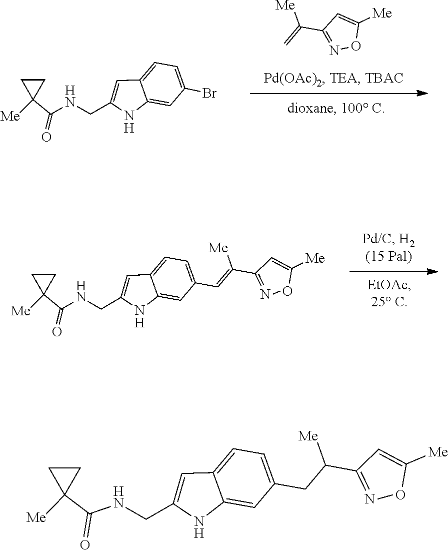

### Step 1: (E)-1-methyl-N-((6-(2-(5-methylisoxazol-3-yl)prop-1-en-1-yl)-1H-indol-2-yl)methyl)cyclopropanecarboxamide

Three reactions were carried out in parallel. To a solution of 5-methyl-3-(prop-1-en-2-yl)isoxazole (601 mg, 4.88 mmol, 5.00 eq) and N-((6-bromo-1H-indol-2-yl)methyl)-1-methylcyclopropanecarboxamide (300 mg, 976 μmol, 1.00 eq) in dioxane (8 mL) were added TEA (395 mg, 3.91 mmol, 4.00 eq), tetrabutylammonium chloride (298 mg, 1.07 mmol, 1.10 eq), and Pd(OAc)₂ (11.0 mg, 48.8 μmol, 0.05 eq) under N₂, and the resulting reaction mixture was heated to 100° C. and stirred under N₂ for 16 h. The three reactions were allowed to cool to room temperature and then were combined for work up. The combined reaction mixture was diluted with H₂O (50 mL) and extracted with ethyl acetate (3×30 mL). The combined organic layers were washed with brine (50 mL), dried over Na₂SO₄, filtered, and concentrated under reduced pressure. Purification by prep-HPLC gave (E)-1-methyl-N-((6-(2-(5-methylisoxazol-3-yl)prop-1-en-1-yl)-1H-indol-2-yl)methyl)cyclopropanecarboxamide. LC-MS (ESI) m/z calcd for C₂₁H₂₃N₃O₂: 349.18; found 350.1 [M+H]⁺.

### Step 2: 1-methyl-N-((6-(2-(5-methylisoxazol-3-yl)propyl)-1H-indol-2-yl)methyl)cyclopropanecarboxamide

To a solution of (E)-1-methyl-N-((6-(2-(5-methylisoxazol-3-yl)prop-1-en-1-yl)-1H-indol-2-yl)methyl)cyclopropanecarboxamide (75.0 mg, 214 μmol, 1.00 eq) in ethyl acetate (10 mL) was added Pd/C (300 mg, 10% by weight), and the reaction mixture was stirred for 16 h under H₂ (15 Psi). The reaction mixture was filtered through Celite and concentrated under reduced pressure. Purification by prep-HPLC gave 1-methyl-N-((6-(2-(5-methylisoxazol-3-yl)propyl)-1H-indol-2-yl)methyl)cyclopropanecarboxamide (Compound 195). LC-MS (ESI) m/z calcd for C₂₁H₂₅N₃O₂: 351.19; found: 352.3 [M+H]⁺. ¹H NMR (400 MHz, DMSO-d₆) δ 10.64 (s, 1H), 8.05-7.92 (m, 1H), 7.30 (d, J=7.6 Hz, 1H), 7.10 (s, 1H), 6.77 (d, J=8.0 Hz, 1H), 6.19 (s, 1H), 6.13 (s, 1H), 4.36 (d, J=5.2 Hz, 2H), 3.17-3.07 (m, 1H), 3.03-2.94 (m, 1H), 2.83-2.74 (m, 1H), 2.33 (s, 3H), 1.29 (s, 3H), 1.14 (d, J=6.0 Hz, 3H), 1.01-0.98 (m, 2H), 0.53-0.51 (m, 2H).

### Example 38: (Compound 196)

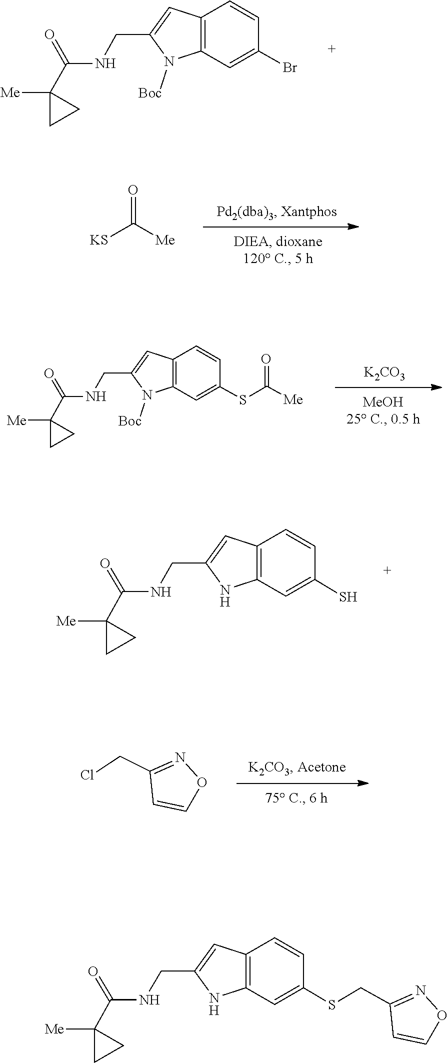

### Step 1: tert-butyl 6-(acetylthio)-2-((1-methylcyclopropanecarboxamido)methyl)-1H-indole-1-carboxylate

To a mixture of tert-butyl 6-bromo-2-((1-methylcyclopropanecarboxamido)methyl)-1H-indole-1-carboxylate (1.00 g, 2.46 mmol, 1.00 eq) and potassium ethanethioate (420 mg, 3.68 mmol, 1.50 eq) in dioxane (10 mL) were added DIEA (855 μL, 634 mg, 4.91 mmol, 2.00 eq), Xantphos (284 mg, 491 μmol, 0.20 eq), and Pd₂(dba)₃ (224 mg, 245 μmol, 0.10 eq) under N₂. The resulting mixture was heated to 120° C. and stirred for 5 h. The reaction mixture was allowed to cool to room temperature, then was quenched by addition of H₂O (20 mL) and extracted with EtOAc (3×20 mL). The combined organic layers were dried over Na₂SO₄, filtered, and concentrated under reduced pressure. Purification by column chromatography gave tert-butyl 6-(acetylthio)-2-((1-methylcyclopropanecarboxamido)methyl)-1H-indole-1-carboxylate. LC-MS (ESI) m/z calcd for C₂₁H₂₆N₂O₄S: 402.16; found: 403.3 [M+H]⁺.

### Step 2: N-((6-mercapto-1H-indol-2-yl)methyl)-1-methylcyclopropanecarboxamide

To a solution of tert-butyl 6-(acetylthio)-2-((1-methylcyclopropanecarboxamido)methyl)-1H-indole-1-carboxylate (230 mg, 571 μmol, 1.00 eq) in MeOH (1 mL) was added K₂CO₃ (236 mg, 1.71 mmol, 3.00 eq) under N₂. The mixture was stirred for 30 min, then was quenched by addition of H₂O (30 mL) and extracted with EtOAc (3×10 mL). The combined organic layers were dried over Na₂SO₄, filtered, and concentrated under reduced pressure. The residue was purified by column chromatography to give N-((6-mercapto-1H-indol-2-yl)methyl)-1-methylcyclopropanecarboxamide. LC-MS (ESI) m/z calcd for C₁₄H₁₆N₂OS: 260.10; found: 261.2 [M+H]⁺.

### Step 3: N-((6-((isoxazol-3-ylmethyl)thio)-1H-indol-2-yl)methyl)-1-methylcyclopropanecarboxamide

To a mixture of N-((6-mercapto-1H-indol-2-yl)methyl)-1-methylcyclopropanecarboxamide (137 mg, 526 μmol, 1.00 eq) and 3-(chloromethyl)isoxazole (61.8 mg, 526 μmol, 1.00 eq) in acetone (3 mL) was added K₂CO₃ (218 mg, 1.58 mmol, 3.00 eq) under N₂. The mixture was heated to 75° C. and stirred for 6 h. The reaction mixture was allowed to cool to room temperature, then was quenched by addition of H₂O (30 mL) and extracted with EtOAc (3×10 mL). The combined organic phase was dried over anhydrous Na₂SO₄, filtered, and concentrated under reduced pressure. Purification by prep-TLC gave N-((6-((isoxazol-3-ylmethyl)thio)-1H-indol-2-yl)methyl)-1-methylcyclopropanecarboxamide (Compound 196). LC-MS (ESI) m/z calcd for C₁₈H₁₉N₃O₂: 341.12; found: 342.1 [M+H]⁺. ¹H NMR (400 MHz, DMSO-d₆) δ 10.86 (s, 1H), 8.77 (d, J=1.6 Hz, 1H), 8.04-8.01 (m, 1H), 7.40-7.34 (m, 2H), 6.99-6.97 (m, 1H), 6.43 (d, J=1.6 Hz, 1H), 6.19 (d, J=6.4 Hz, 1H), 4.38 (d, J=6.0 Hz, 2H), 4.16 (s, 2H), 1.29 (s, 3H), 1.01-0.99 (m, 2H), 0.55-0.52 (m, 2H).

### Example 39: (Compound 197)

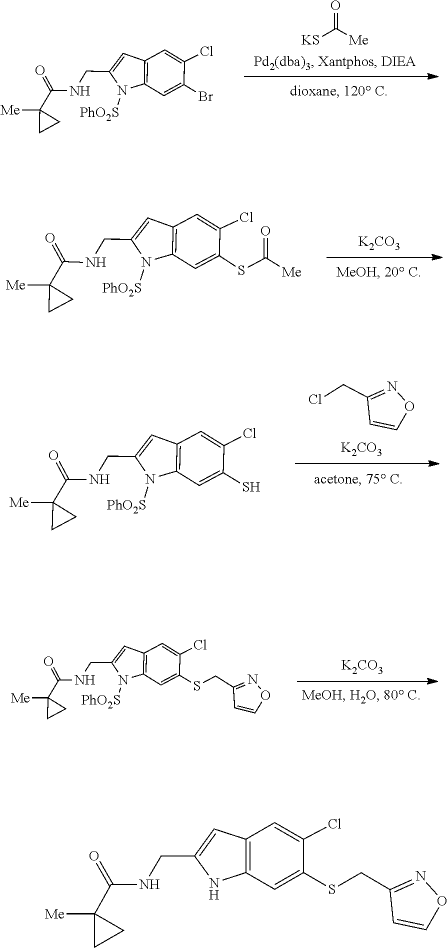

### Step 1: S-(5-chloro-2-((1-methylcyclopropanecarboxamido)methyl)-1-(phenylsulfonyl)-1H-indol-6-yl) ethanethioate

To a mixture of N-((6-bromo-5-chloro-1-(phenylsulfonyl)-1H-indol-2-yl)methyl)-1-methylcyclopropanecarboxamide (690 mg, 1.43 mmol, 1.00 eq) and potassium ethanethioate (245 mg, 2.15 mmol, 1.50 eq) in dioxane (10 mL) were added DIEA (498 μL, 370 mg, 2.86 mmol, 2.00 eq), Xantphos (165 mg, 286 μmol, 0.20 eq), and Pd₂(dba)₃ (131 mg, 143 μmol, 0.10 eq) under N₂. The mixture was heated to 120° C. and stirred for 5 h. The reaction mixture was allowed to cool to room temperature, then was quenched by addition of H₂O (20 mL) and extracted with EtOAc (3×10 mL). The combined organic layers were dried over Na₂SO₄, filtered, and concentrated under reduced pressure. Purification by column chromatography gave S-(5-chloro-2-((1-methylcyclopropanecarboxamido)methyl)-1-(phenylsulfonyl)-1H-indol-6-yl) ethanethioate. LC-MS (ESI) m/z calcd for C₂₂H₂₁ClN₂O₄S₂: 476.06; found: 477.2 [M+H]⁺.

### Step 2: N-((5-chloro-6-mercapto-1-(phenylsulfonyl)-1H-indol-2-yl)methyl)-1-methyl cyclopropanecarboxamide

To a solution of S-(5-chloro-2-((1-methylcyclopropanecarboxamido)methyl)-1-(phenylsulfonyl)-1H-indol-6-yl) ethanethioate (220 mg, 461 μmol, 1.00 eq) in MeOH (3 mL) was added K₂CO₃ (191 mg, 1.38 mmol, 3.00 eq) under N₂. The mixture was stirred for 0.5 h, then was quenched by addition of H₂O (20 mL) and extracted with EtOAc (3×10 mL). The combined organic layers were dried over Na₂SO₄, filtered, and concentrated under reduced pressure to give N-((5-chloro-6-mercapto-1-(phenylsulfonyl)-1H-indol-2-yl)methyl)-1-methylcyclopropanecarboxamide, which was used without further purification. LC-MS (ESI) m/z calcd for C₂₀H₁₉ClN₂O₃S₂: 434.04; found: 435.0 [M+H]⁺.

### Step 3: N-((5-chloro-6-((isoxazol-3-ylmethyl)thio)-1-(phenylsulfonyl)-1H-indol-2-yl)methyl)-1-methylcyclopropanecarboxamide

To a mixture of N-((5-chloro-6-mercapto-1-(phenylsulfonyl)-1H-indol-2-yl)methyl)-1-methylcyclopropanecarboxamide (200 mg, 459 μmol, 1.00 eq) and 3-(chloromethyl)isoxazole (54.0 mg, 459 μmol, 1.00 eq) in acetone (3 mL) was added K₂CO₃ (190 mg, 1.38 mmol, 3.00 eq) under N₂. The mixture was heated to 75° C. and stirred for 4 h. The reaction mixture was allowed to cool to room temperature, then was quenched by addition of H₂O (20 mL) and extracted with EtOAc (3×10 mL). The combined organic layers were dried over Na₂SO₄, filtered, and concentrated under reduced pressure to give N-((5-chloro-6-((isoxazol-3-ylmethyl)thio)-1-(phenylsulfonyl)-1H-indol-2-yl)methyl)-1-methylcyclopropanecarboxamide, which was used without further purification. LC-MS (ESI) m/z calcd for C₂₄H₂₂ClN₃O₄S₂: 515.07; found: 516.1 [M+H]⁺.

### Step 4: N-((5-chloro-6-((isoxazol-3-ylmethyl)thio)-1H-indol-2-yl)methyl)-1-methylcyclopropanecarboxamide

To a solution of N-((5-chloro-6-((isoxazol-3-ylmethyl)thio)-1-(phenylsulfonyl)-1H-indol-2-yl)methyl)-1-methylcyclopropanecarboxamide (200 mg, 387 μmol, 1.00 eq) in MeOH (3 mL) and H₂O (1 mL) was added K₂CO₃ (267 mg, 1.94 mmol, 5.00 eq) under N₂. The mixture was heated to 80° C. and stirred for 2 h. The reaction mixture was allowed to cool to room temperature, then was quenched by addition of H₂O (20 mL) and extracted with EtOAc (3×10 mL). The combined organic layers were dried over Na₂SO₄, filtered, and concentrated under reduced pressure. Purification by prep-HPLC gave N-((5-chloro-6-((isoxazol-3-ylmethyl)thio)-1H-indol-2-yl) methyl)-1-methylcyclopropanecarboxamide (Compound 197). LC-MS (ESI) m/z calcd for C₁₈H₁₈ClN₃O₂S: 375.08; found: 376.0 [M+H]⁺. ¹H NMR (400 MHz, DMSO-d₆) δ 11.03 (s, 1H), 8.79 (d, J=1.2 Hz, 1H), 8.06 (t, J=6.0 Hz, 1H), 7.58 (s, 1H), 7.43 (s, 1H), 6.50 (d, J=1.6 Hz, 1H), 6.18 (s, 1H), 4.37 (d, J=5.6 Hz, 2H), 4.23 (s, 2H), 1.29 (s, 3H), 1.00-0.98 (m, 2H), 0.55-0.52 (m, 2H).

### Example 40: (Compound 198)

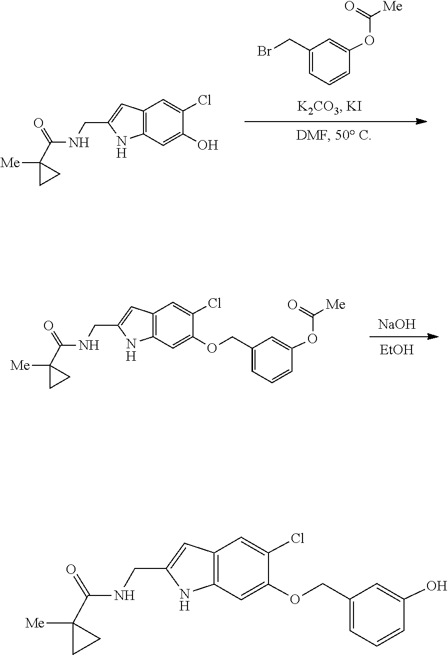 [SMILES: "EtOH" ; CC(=O)Oc1cccc(CBr)c1 ; CC1(C(=O)NCc2cc3cc(Cl)c(O)cc3[nH]2)CC1 ; CC(=O)Oc1cccc(COc2cc3[nH]c(CNC(=O)C4(C)CC4)cc3cc2Cl)c1 ; "NaOH" ; CC1(C(=O)NCc2cc3cc(Cl)c(OCc4cccc(O)c4)cc3[nH]2)CC1]

### Step 1: 3-(((5-chloro-2-((1-methylcyclopropane-1-carboxamido)methyl)-1H-indol-6-yl)oxy) methyl)phenyl acetate

A mixture of 3-(bromomethyl)phenyl acetate (430 mg, 1.88 mmol, 1.00 eq), N-[(5-chloro-6-hydroxy-1H-indol-2-yl)methyl]-1-methyl-cyclopropanecarboxamide (418 mg, 1.50 mmol, 0.80 eq), K₂CO₃ (778 mg, 5.63 mmol, 3.00 eq), and KI (467 mg, 2.82 mmol, 1.50 eq) in DMF (5 mL) was degassed and purged with N₂ three times. The mixture was then heated to 50° C. and stirred for 2 h under N₂ atmosphere. The reaction mixture was allowed to cool to room temperature, then was quenched by addition of H₂O (20 mL) and extracted with ethyl acetate (3×10 mL). The combined organic layers were dried over Na₂SO₄, filtered, and concentrated under reduced pressure. Purification by column chromatography gave 3-(((5-chloro-2-((1-methylcyclopropane-1-carboxamido)methyl)-1H-indol-6-yl)oxy)methyl)phenyl acetate. LC-MS (ESI) m/z calcd for C₂₃H₂₃ClN₂O₄: 426.13; found: 427.2 [M+H]⁺.

### Step 2: N-((5-chloro-6-((3-hydroxybenzyl) oxy)-1H-indol-2-yl) methyl)-1-methylcyclopropane-1-carboxamide

A mixture of 3-(((5-chloro-2-((1-methylcyclopropane-1-carboxamido)methyl)-1H-indol-6-yl)oxy)methyl)phenyl acetate (100 mg, 234 μmol, 1.00 eq), and NaOH (9.37 mg, 234 μmol, 1.00 eq) in ethyl alcohol (3 mL) was degassed and purged with N₂ three times, and the mixture was heated to 50° C. and stirred for 2 h under N₂ atmosphere. The reaction mixture was allowed to cool to room temperature, then was quenched by addition of H₂O (5 mL) and extracted with ethyl acetate (3×10 mL). The combined organic layers were dried over Na₂SO₄, filtered, and concentrated under reduced pressure. Purification by prep-HPLC gave N-((5-chloro-6-((3-hydroxybenzyl)oxy)-1H-indol-2-yl)methyl)-1-methylcyclopropane-1-carboxamide (Compound 198). LC-MS (ESI) m/z calcd for C₂₁H₂₁ClN₂O₃: 384.12; found 385.1 [M+H]⁺. ¹H NMR (400 MHz, DMSO-d₆) δ 10.74 (s, 1H), 9.44 (s, 1H), 8.01 (t, J=5.6 Hz, 1H), 7.49 (s, 1H), 7.17 (d, J=8.0 Hz, 1H), 7.07 (s, 1H), 6.89 (d, J=6.0 Hz, 2H), 6.71-6.68 (m, 1H), 6.11 (s, 1H), 5.08 (s, 2H), 4.34 (d, J=5.2 Hz, 2H), 1.28 (s, 3H), 1.00-0.97 (m, 2H), 0.54-0.51 (m, 2H).

The following compounds in Table T-20 were synthesized using procedures similar to Compound 198 using the appropriate starting materials.

TABLE T-20

| | | | Exact | LCMS, |
|---|---|---|---|---|
| Compound | | | mass | found |
| No. | Structure | IUPAC name | (g/mol) | [M + H]⁺ |
| 203 | 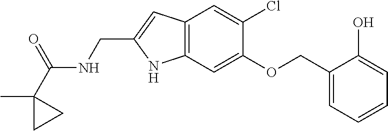 [SMILES: CC1(C(=O)NCc2cc3cc(Cl)c(OCc4ccccc4O)cc3[nH]2)CC1] | N-((5-chloro-6-((2- hydroxybenzyl)oxy)- 1H-indol-2- yl)methyl)-1- methylcyclopropane- 1-carboxamide | 384.12 | 385.1 |

### Example 41: (Compound 209)

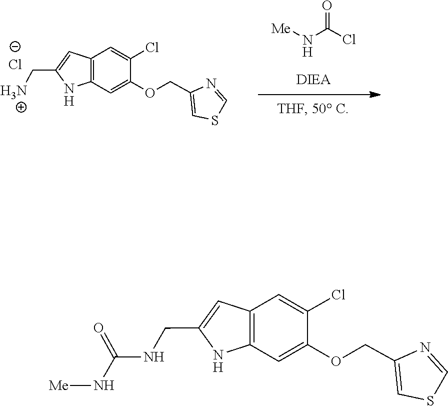 [SMILES: CNC(=O)Cl ; [Cl-] ; [NH3+]Cc1cc2cc(Cl)c(OCc3cscn3)cc2[nH]1 ; CNC(=O)NCc1cc2cc(Cl)c(OCc3cscn3)cc2[nH]1]

### Step 1: 1-[[5-chloro-6-(thiazol-4-ylmethoxy)-1H-indol-2-yl]methyl]-3-methyl-urea

To a solution of (5-chloro-6-(thiazol-4-ylmethoxy)-1H-indol-2-yl)methanamine hydrochloride (4.00 g, 12.1 mmol, 1.00 eq) in THF (40 mL) were added DIEA (36.3 mmol, 6.33 mL, 3.00 eq) and N-methylcarbamoyl chloride (2.27 g, 24.2 mmol, 2.00 eq). The reaction mixture was heated to 50° C. and stirred for 1 h. The mixture was allowed to cool to room temperature, then was poured into water (30 mL) and stirred for 10 min. The aqueous phase was extracted with ethyl acetate (2×30 mL). The combined organic phase was washed with brine (2×50 mL), dried over anhydrous Na₂SO₄, filtered, and concentrated under reduced pressure. The crude product was triturated with a mixture of MeOH (10 mL) and DMF (4 mL) at 50° C. for 30 m, then was further triturated with MeOH (5 mL) and filtered to give 1-[[5-chloro-6-(thiazol-4-ylmethoxy)-H-indol-2-yl]methyl]-3-methyl-urea (Compound 209). LC-MS (ESI) m/z calcd for C₁₅H₁₅ClN₄O₂S: 350.06; found: 351.1 [M+H]⁺. ¹H NMR (400 MHz, DMSO-d₆) δ 10.84 (s, 1H), 9.14 (s, 1H), 7.75 (s, 1H), 7.48 (s, 1H), 7.17 (s, 1H), 6.31 (t, J=5.2 Hz, 1H), 6.12 (s, 1H), 5.86 (d, J=4.4 Hz, 1H), 5.26 (s, 2H), 4.27 (d, J=5.6 Hz, 2H), 2.58 (d, J=4.8 Hz, 3H).

The following compounds in Table T-21 were synthesized using procedures similar to Compound 209 using the appropriate starting materials.

TABLE T-21

| Com- | | | Exact | LCMS, |
|---|---|---|---|---|
| pound | | | mass | found |
| No. | Structure | IUPAC name | (g/mol) | [M + H]⁺ |
| 210 | 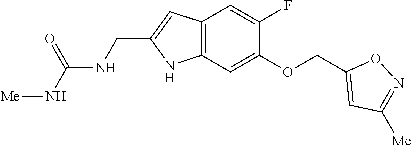 [SMILES: CNC(=O)NCc1cc2cc(F)c(OCc3cc(C)no3)cc2[nH]1] | 1-((5-fluoro-6-((3- methylisoxazol-5- yl)methoxy)-1H-indol-2- yl)methyl)-3-methylurea | 332.13 | 333.1 |
| 211 | 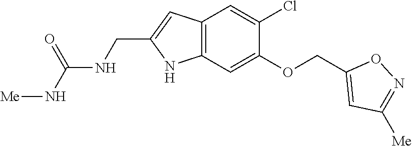 [SMILES: CNC(=O)NCc1cc2cc(Cl)c(OCc3cc(C)no3)cc2[nH]1] | 1-((5-chloro-6-((3- methylisoxazol-5- yl)methoxy)-1H-indol-2- yl)methyl)-3-methylurea | 348.10 | 349.1 |
| 212 | 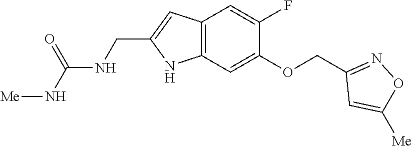 [SMILES: CNC(=O)NCc1cc2cc(F)c(OCc3cc(C)on3)cc2[nH]1] | 1-((5-fluoro-6-((5- methylisoxazol-3- yl)methoxy)-1H-indol-2- yl)methyl)-3-methylurea | 332.13 | 333.1 |
| 213 | 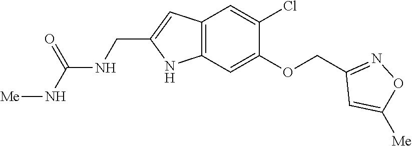 [SMILES: CNC(=O)NCc1cc2cc(Cl)c(OCc3cc(C)on3)cc2[nH]1] | 1-((5-chloro-6-((5- methylisoxazol-3- yl)methoxy)-1H-indol-2- yl)methyl)-3-methylurea | 348.10 | 349.2 |
| 214 | 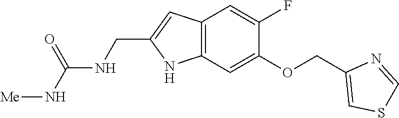 [SMILES: CNC(=O)NCc1cc2cc(F)c(OCc3cscn3)cc2[nH]1] | 1-((5-fluoro-6-(thiazol-4- ylmethoxy)-1H-indol-2- yl)methyl)-3-methylurea | 334.09 | 335.2 |
| 215 | 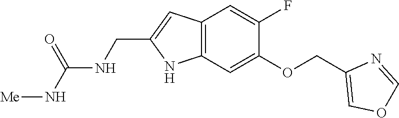 [SMILES: CNC(=O)NCc1cc2cc(F)c(OCc3cocn3)cc2[nH]1] | 1-((5-fluoro-6-(oxazol-4- ylmethoxy)-1H-indol-2- yl)methyl)-3-methylurea | 318.11 | 319.3 |
| 216 | 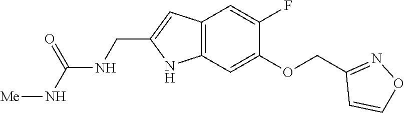 [SMILES: CNC(=O)NCc1cc2cc(F)c(OCc3ccon3)cc2[nH]1] | 1-((5-fluoro-6-(isoxazol- 3-ylmethoxy)-1H-indol- 2-yl)methyl)-3- methylurea | 318.11 | 319.3 |
| 217 | 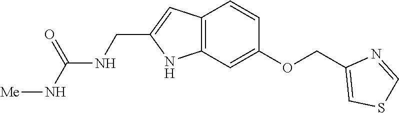 [SMILES: CNC(=O)NCc1cc2ccc(OCc3cscn3)cc2[nH]1] | 1-methyl-3-((6-(thiazol-4- ylmethoxy)-1H-indol-2- yl)methyl)urea | 316.10 | 317.0 |
| 467 | 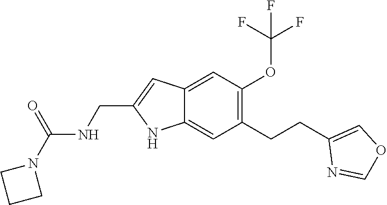 [SMILES: O=C(NCc1cc2cc(OC(F)(F)F)c(CCc3cocn3)cc2[nH]1)N1CCC1] | N-((6-(2-(oxazol-4- yl)ethyl)-5- (trifluoromethoxy)-1H- indol-2- yl)methyl)azetidine-1- carboxamide | 408.14 | 409.1 |
| 468 | 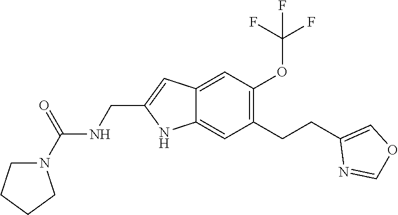 [SMILES: O=C(NCc1cc2cc(OC(F)(F)F)c(CCc3cocn3)cc2[nH]1)N1CCCC1] | N-((6-(2-(oxazol-4- yl)ethyl)-5- (trifluoromethoxy)-1H- indol-2- yl)methyl)pyrrolidine-1- carboxamide | 422.16 | 423.2 |
| 469 | 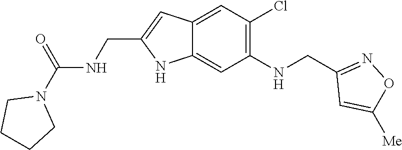 [SMILES: Cc1cc(CNc2cc3[nH]c(CNC(=O)N4CCCC4)cc3cc2Cl)no1] | N-((5-chloro-6-(((5- methylisoxazol-3- yl)methyl)amino)-1H- indol-2- yl)methyl)pyrrolidine-1- carboxamide | 387.15 | 388.3 |
| 470 | 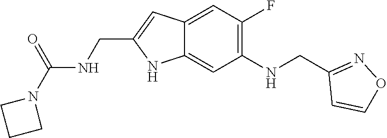 [SMILES: O=C(NCc1cc2cc(F)c(NCc3ccon3)cc2[nH]1)N1CCC1] | N-((5-fluoro-6-((isoxazol- 3-ylmethyl)amino)-1H- indol-2- yl)methyl)azetidine-1- carboxamide | 343.14 | 344.2 |
| 471 | 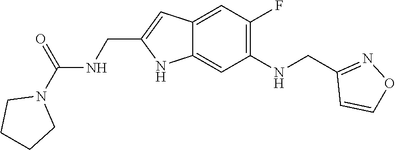 [SMILES: O=C(NCc1cc2cc(F)c(NCc3ccon3)cc2[nH]1)N1CCCC1] | N-((5-fluoro-6-((isoxazol- 3-ylmethyl)amino)-1H- indol-2- yl)methyl)pyrrolidine-1- carboxamide | 357.16 | 358.2 |
| 472 | 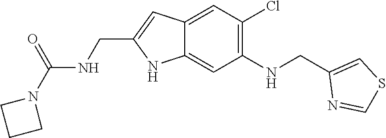 [SMILES: O=C(NCc1cc2cc(Cl)c(NCc3cscn3)cc2[nH]1)N1CCC1] | N-((5-chloro-6-((thiazol- 4-ylmethyl)amino)-1H- indol-2- yl)methyl)azetidine-1- carboxamide | 375.09 | 376.2 |
| 473 | 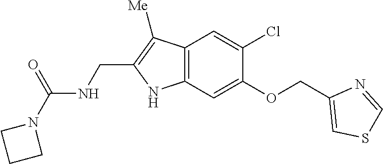 [SMILES: Cc1c(CNC(=O)N2CCC2)[nH]c2cc(OCc3cscn3)c(Cl)cc12] | N-((5-chloro-3-methyl-6- (thiazol-4-ylmethoxy)- 1H-indol-2- yl)methyl)azetidine-1- carboxamide | 390.09 | 391.3 |
| 474 | 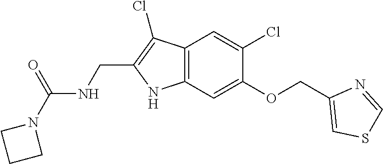 [SMILES: O=C(NCc1[nH]c2cc(OCc3cscn3)c(Cl)cc2c1Cl)N1CCC1] | N-((3,5-dichloro-6- (thiazol-4-ylmethoxy)- 1H-indol-2- yl)methyl)azetidine-1- carboxamide | 410.04 | 411.2 |
| 475 | 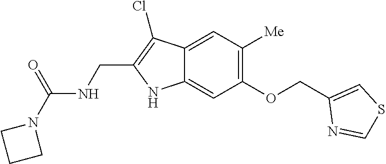 [SMILES: Cc1cc2c(Cl)c(CNC(=O)N3CCC3)[nH]c2cc1OCc1cscn1] | N-((3-chloro-5-methyl-6- (thiazol-4-ylmethoxy)- 1H-indol-2- yl)methyl)azetidine-1- carboxamide | 390.09 | 391.3 |
| 476 | 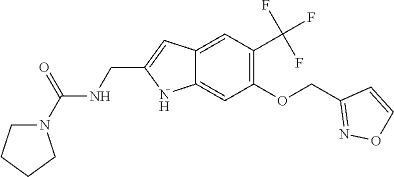 [SMILES: O=C(NCc1cc2cc(C(F)(F)F)c(OCc3ccon3)cc2[nH]1)N1CCCC1] | N-((6-(isoxazol-3- ylmethoxy)-5- (trifluoromethyl)-1H- indol-2- yl)methyl)pyrrolidine-1- carboxamide | 408.14 | 409.1 |
| 477 | 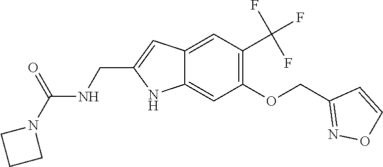 [SMILES: O=C(NCc1cc2cc(C(F)(F)F)c(OCc3ccon3)cc2[nH]1)N1CCC1] | N-((6-(isoxazol-3- ylmethoxy)-5- (trifluoromethyl)-1H- indol-2- yl)methyl)azetidine-1- carboxamide | 394.13 | 395.1 |
| 478 | 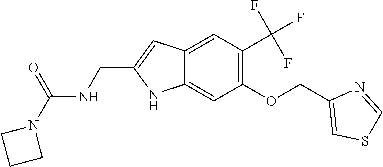 [SMILES: O=C(NCc1cc2cc(C(F)(F)F)c(OCc3cscn3)cc2[nH]1)N1CCC1] | N-((6-(thiazol-4- ylmethoxy)-5- (trifluoromethyl)-1H- indol-2- yl)methyl)azetidine-1- carboxamide | 410.10 | 411.3 |
| 479 | 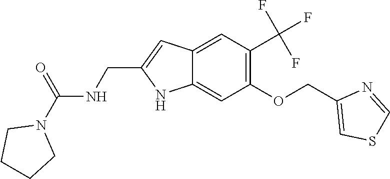 [SMILES: O=C(NCc1cc2cc(C(F)(F)F)c(OCc3cscn3)cc2[nH]1)N1CCCC1] | N-((6-(thiazol-4- ylmethoxy)-5- (trifluoromethyl)-1H- indol-2- yl)methyl)pyrrolidine-1- carboxamide | 424.12 | 425.3 |
| 480 | 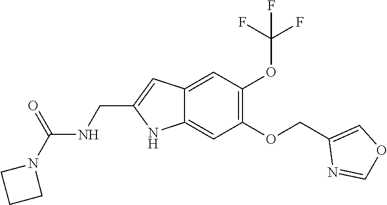 [SMILES: O=C(NCc1cc2cc(OC(F)(F)F)c(OCc3cocn3)cc2[nH]1)N1CCC1] | N-((6-(oxazol-4- ylmethoxy)-5- (trifluoromethoxy)-1H- indol-2- yl)methyl)azetidine-1- carboxamide | 410.12 | 411.3 |
| 481 | 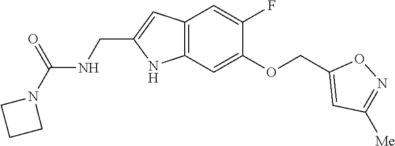 [SMILES: Cc1cc(COc2cc3[nH]c(CNC(=O)N4CCC4)cc3cc2F)on1] | N-((5-fluoro-6-((3- methylisoxazol-5- yl)methoxy)-1H-indol-2- yl)methyl)azetidine-1- carboxamide | 358.14 | 358.9 |

### Example 42: (Compound 218)

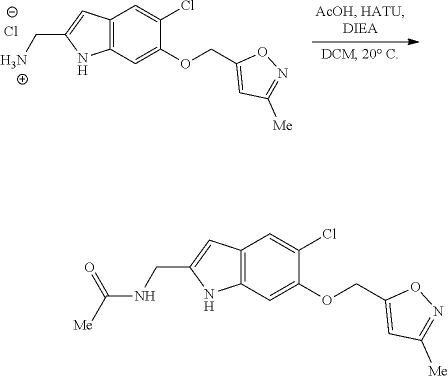 [SMILES: [Cl-] ; Cc1cc(COc2cc3[nH]c(C[NH3+])cc3cc2Cl)on1 ; CC(=O)NCc1cc2cc(Cl)c(OCc3cc(C)no3)cc2[nH]1]

### Step 1: N-((5-chloro-6-((3-methylisoxazol-5-yl)methoxy)-1H-indol-2-yl)methyl)acetamide

To a solution of AcOH (400 μmol, 19.1 μL, 1.10 eq) in DCM (1 mL) was added HATU (173 mg, 546 μmol, 1.50 eq). The reaction mixture was stirred at 20° C. for 0.5 h, then DIEA (1.45 mmol, 212 μL, 4.00 eq) and (5-chloro-6-((3-methylisoxazol-5-yl)methoxy)-1H-indol-2-yl)methanamine hydrochloride (120 mg, 364 μmol, 1.00 eq) was added. The reaction mixture was stirred at 20° C. for 1 h, then was quenched by addition of water (3 mL) and extracted with ethyl acetate (3×10 mL). The combined organic layers were dried over anhydrous Na₂SO₄, filtered, and concentrated under reduced pressure. Purification by prep-HPLC gave N-((5-chloro-6-((3-methylisoxazol-5-yl)methoxy)-1H-indol-2-yl)methyl)acetamide (Compound 218). LC-MS (ESI) m/z calcd for C₁₆H₁₆ClN₃O₃: 333.09; found: 334.3 [M+H]⁺. ¹H NMR (400 MHz, DMSO-d₆) δ 10.95 (s, 1H) 8.32 (t, J=5.2 Hz, 1H) 7.51 (s, 1H) 7.14 (s, 1H) 6.46 (s, 1H) 6.18 (s, 1H) 5.28 (s, 2H) 4.34 (d, J=5.2 Hz, 2H) 2.24 (s, 3H) 1.87 (s, 3H).

The following compounds in Table T-22 were synthesized using procedures similar to Compound 218 using the appropriate starting materials.

TABLE T-22

| Com- | | | Exact | LCMS, |
|---|---|---|---|---|
| pound | | | mass | found |
| No. | Structure | IUPAC name | (g/mol) | [M + H]⁺ |
| 219 | 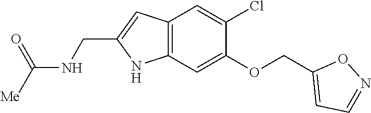 [SMILES: CC(=O)NCc1cc2cc(Cl)c(OCc3ccno3)cc2[nH]1] | N-((5-chloro-6-(isoxazol-5- ylmethoxy)-1H-indol-2- yl)methyl)acetamide | 319.07 | 320.1 |
| 220 | 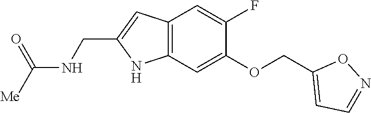 [SMILES: CC(=O)NCc1cc2cc(F)c(OCc3ccno3)cc2[nH]1] | N-((5-fluoro-6-(isoxazol-5- ylmethoxy)-1H-indol-2- yl)methyl)acetamide | 303.10 | 304.2 |
| 221 | 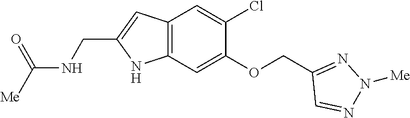 [SMILES: CC(=O)NCc1cc2cc(Cl)c(OCc3cnn(C)n3)cc2[nH]1] | N-((5-chloro-6-((2-methyl-2H- 1,2,3-triazol-4-yl)methoxy)- 1H-indol-2- yl)methyl)acetamide | 333.10 | 334.1 |
| 222 | 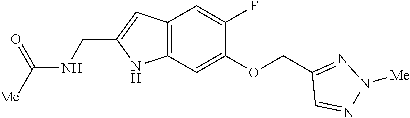 [SMILES: CC(=O)NCc1cc2cc(F)c(OCc3cnn(C)n3)cc2[nH]1] | N-((5-fluoro-6-((2-methyl-2H- 1,2,3-triazol-4-yl)methoxy)- 1H-indol-2- yl)methyl)acetamide | 317.13 | 318.1 |
| 223 | 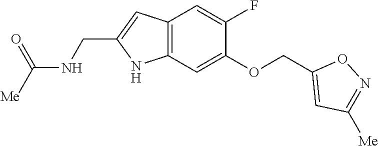 [SMILES: CC(=O)NCc1cc2cc(F)c(OCc3cc(C)no3)cc2[nH]1] | N-((5-fluoro-6-((3- methylisoxazol-5-yl)methoxy)- 1H-indol-2- yl)methyl)acetamide | 317.12 | 318.3 |
| 224 | 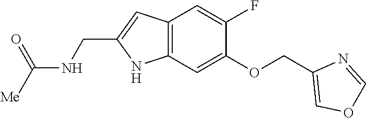 [SMILES: CC(=O)NCc1cc2cc(F)c(OCc3cocn3)cc2[nH]1] | N-((5-fluoro-6-(oxazol-4- ylmethoxy)-1H-indol-2- yl)methyl)acetamide | 303.10 | 304.1 |
| 225 | 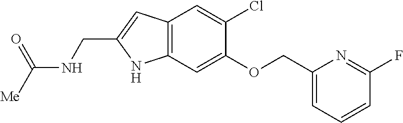 [SMILES: CC(=O)NCc1cc2cc(Cl)c(OCc3cccc(F)n3)cc2[nH]1] | N-((5-chloro-6-((6- fluoropyridin-2-yl)methoxy)- 1H-indol-2- yl)methyl)acetamide | 347.08 | 348.2 |
| 226 | 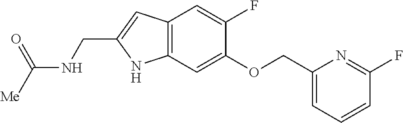 [SMILES: CC(=O)NCc1cc2cc(F)c(OCc3cccc(F)n3)cc2[nH]1] | N-((5-fluoro-6-((6- fluoropyridin-2-yl)methoxy)- 1H-indol-2- yl)methyl)acetamide | 331.11 | 332.3 |
| 227 | 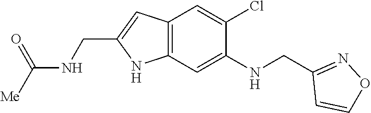 [SMILES: CC(=O)NCc1cc2cc(Cl)c(NCc3ccon3)cc2[nH]1] | N-((5-chloro-6-((isoxazol-3- ylmethyl)amino)-1H-indol-2- yl)methyl)acetamide | 318.09 | 319.2 |
| 228 | 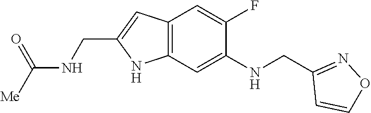 [SMILES: CC(=O)NCc1cc2cc(F)c(NCc3ccon3)cc2[nH]1] | N-((5-fluoro-6-((isoxazol-3- ylmethyl)amino)-1H-indol-2- yl)methyl)acetamide | 302.12 | 303.2 |
| 229 | 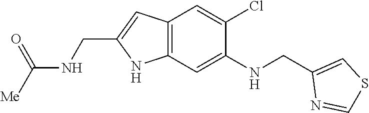 [SMILES: CC(=O)NCc1cc2cc(Cl)c(NCc3cscn3)cc2[nH]1] | N-((5-chloro-6-((thiazol-4- ylmethyl)amino)-1H-indol-2- yl)methyl)acetamide | 334.07 | 335.2 |
| 246 | 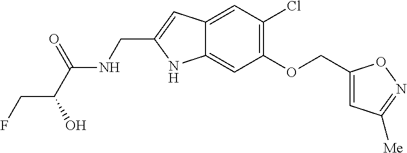 [SMILES: Cc1cc(COc2cc3[nH]c(CNC(=O)[C@H](O)CF)cc3cc2Cl)on1] | (S)-N-((5-chloro-6-((3- methylisoxazol-5-yl)methoxy)- 1H-indol-2-yl)methyl)-3- fluoro-2-hydroxypropanamide | 381.09 | 382.1 |
| 247 | 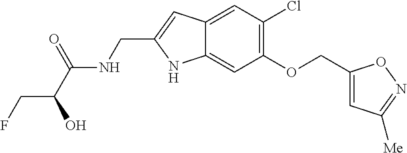 [SMILES: Cc1cc(COc2cc3[nH]c(CNC(=O)[C@@H](O)CF)cc3cc2Cl)on1] | (R)-N-((5-chloro-6-((3- methylisoxazol-5-yl)methoxy)- 1H-indol-2-yl)methyl)-3- fluoro-2-hydroxypropanamide | 381.09 | 382.1 |
| 248 | 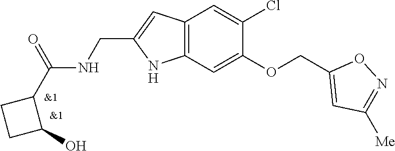 [SMILES: Cc1cc(COc2cc3[nH]c(CNC(=O)C4CC[C@@H]4O)cc3cc2Cl)on1] | (1R,2R)-N-((5-chloro-6-((3- methylisoxazol-5-yl)methoxy)- 1H-indol-2-yl)methyl)-2- hydroxycyclobutane-1- carboxamide and (1S,2S)-N- ((5-chloro-6-((3- methylisoxazol-5-yl)methoxy)- 1H-indol-2-yl)methyl)-2- hydroxycyclobutane-1- carboxamide | 389.11 | 390.2 |
| 249 | 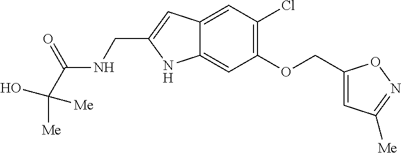 [SMILES: Cc1cc(COc2cc3[nH]c(CNC(=O)C(C)(C)O)cc3cc2Cl)on1] | N-((5-chloro-6-((3- methylisoxazol-5-yl)methoxy)- 1H-indol-2-yl)methyl)-2- hydroxy-2-methylpropanamide | 377.11 | 378.2 |
| 250 | 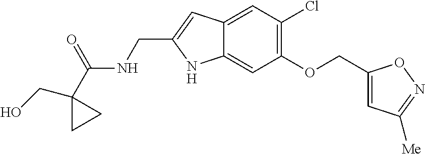 [SMILES: Cc1cc(COc2cc3[nH]c(CNC(=O)C4(CO)CC4)cc3cc2Cl)on1] | N-((5-chloro-6-((3- methylisoxazol-5-yl)methoxy)- 1H-indol-2-yl)methyl)-1- (hydroxymethyl)cyclopropane- 1-carboxamide | 389.11 | 390.2 |
| 251 | 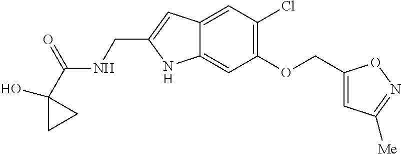 [SMILES: Cc1cc(COc2cc3[nH]c(CNC(=O)C4(O)CC4)cc3cc2Cl)on1] | N-((5-chloro-6-((3- methylisoxazol-5-yl)methoxy)- 1H-indol-2-yl)methyl)-1- hydroxycyclopropane-1- carboxamide | 375.10 | 376.2 |
| 252 | 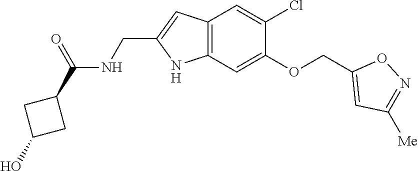 [SMILES: Cc1cc(COc2cc3[nH]c(CNC(=O)[C@H]4C[C@H](O)C4)cc3cc2Cl)on1] | (1r,3r)-N-((5-chloro-6-((3- methylisoxazol-5-yl)methoxy)- 1H-indol-2-yl)methyl)-3- hydroxycyclobutane-1- carboxamide | 389.11 | 390.2 |
| 253 | 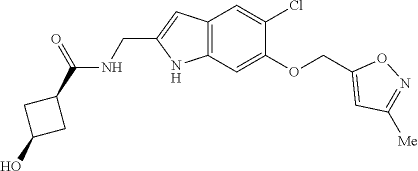 [SMILES: Cc1cc(COc2cc3[nH]c(CNC(=O)[C@H]4C[C@@H](O)C4)cc3cc2Cl)on1] | (1s,3s)-N-((5-chloro-6-((3- methylisoxazol-5-yl)methoxy)- 1H-indol-2-yl)methyl)-3- hydroxycyclobutane-1- carboxamide | 389.11 | 390.2 |
| 254 | 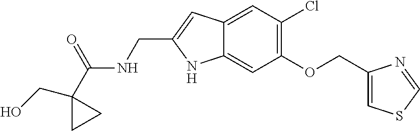 [SMILES: O=C(NCc1cc2cc(Cl)c(OCc3cscn3)cc2[nH]1)C1(CO)CC1] | N-((5-chloro-6-(thiazol-4- ylmethoxy)-1H-indol-2- yl)methyl)-1- (hydroxymethyl)cyclopropane- 1-carboxamide | 391.08 | 392.2 |
| 255 | 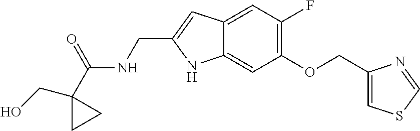 [SMILES: O=C(NCc1cc2cc(F)c(OCc3cscn3)cc2[nH]1)C1(CO)CC1] | N-((5-fluoro-6-(thiazol-4- ylmethoxy)-1H-indol-2- yl)methyl)-1- (hydroxymethyl)cyclopropane- 1-carboxamide | 375.11 | 376.1 |
| 256 | 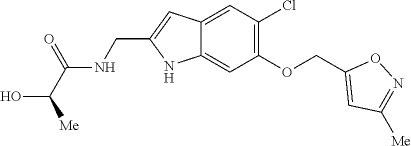 [SMILES: Cc1cc(COc2cc3[nH]c(CNC(=O)[C@@H](C)O)cc3cc2Cl)on1] | (R)-N-((5-chloro-6-((3- methylisoxazol-5-yl)methoxy)- 1H-indol-2-yl)methyl)-2- hydroxypropanamide | 363.10 | 364.1 |
| 257 | 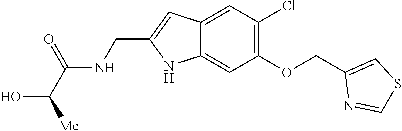 [SMILES: C[C@@H](O)C(=O)NCc1cc2cc(Cl)c(OCc3cscn3)cc2[nH]1] | (R)-N-((5-chloro-6-(thiazol-4- ylmethoxy)-1H-indol-2- yl)methyl)-2- hydroxypropanamide | 365.06 | 366.1 |
| 258 | 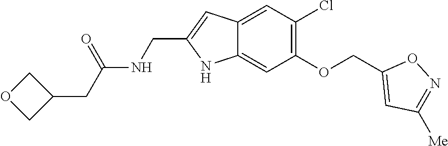 [SMILES: Cc1cc(COc2cc3[nH]c(CNC(=O)CC4COC4)cc3cc2Cl)on1] | N-((5-chloro-6-((3- methylisoxazol-5-yl)methoxy)- 1H-indol-2-yl)methyl)-2- (oxetan-3-yl)acetamide | 389.11 | 390.0 |
| 259 | 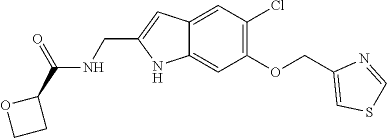 [SMILES: O=C(NCc1cc2cc(Cl)c(OCc3cscn3)cc2[nH]1)[C@H]1CCO1] | (R)-N-((5-chloro-6-(thiazol-4- ylmethoxy)-1H-indol-2- yl)methyl)oxetane-2- carboxamide | 377.06 | 378.1 |
| 260 | 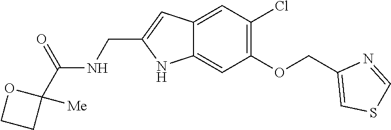 [SMILES: CC1(C(=O)NCc2cc3cc(Cl)c(OCc4cscn4)cc3[nH]2)CCO1] | N-((5-chloro-6-(thiazol-4- ylmethoxy)-1H-indol-2- yl)methyl)-2-methyloxetane-2- carboxamide | 391.08 | 391.9 |
| 261 | 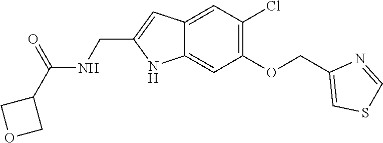 [SMILES: O=C(NCc1cc2cc(Cl)c(OCc3cscn3)cc2[nH]1)C1COC1] | N-((5-chloro-6-(thiazol-4- ylmethoxy)-1H-indol-2- yl)methyl)oxetane-3- carboxamide | 377.06 | 378.0 |
| 262 | 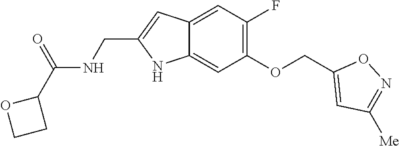 [SMILES: Cc1cc(COc2cc3[nH]c(CNC(=O)C4CCO4)cc3cc2F)on1] | N-((5-fluoro-6-((3- methylisoxazol-5-yl)methoxy)- 1H-indol-2-yl)methyl)oxetane- 2-carboxamide | 359.13 | 360.1 |
| 263 | 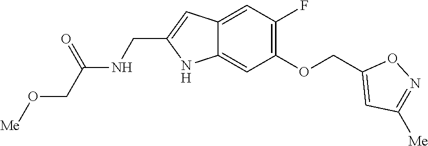 [SMILES: COCC(=O)NCc1cc2cc(F)c(OCc3cc(C)no3)cc2[nH]1] | N-((5-fluoro-6-((3- methylisoxazol-5-yl)methoxy)- 1H-indol-2-yl)methyl)-2- methoxyacetamide | 347.13 | 348.2 |
| 264 | 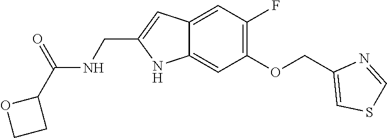 [SMILES: O=C(NCc1cc2cc(F)c(OCc3cscn3)cc2[nH]1)C1CCO1] | N-((5-fluoro-6-(thiazol-4- ylmethoxy)-1H-indol-2- yl)methyl)oxetane-2- carboxamide | 361.09 | 362.1 |
| 265 | 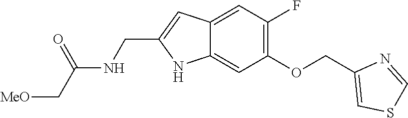 [SMILES: COCC(=O)NCc1cc2cc(F)c(OCc3cscn3)cc2[nH]1] | N-((5-fluoro-6-(thiazol-4- ylmethoxy)-1H-indol-2- yl)methyl)-2- methoxyacetamide | 349.09 | 350.1 |
| 266 | 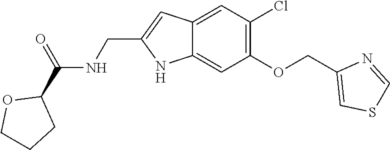 [SMILES: O=C(NCc1cc2cc(Cl)c(OCc3cscn3)cc2[nH]1)[C@H]1CCCO1] | (R)-N-((5-chloro-6-(thiazol-4- ylmethoxy)-1H-indol-2- yl)methyl)tetrahydrofuran-2- carboxamide | 391.08 | 392.1 |
| 267 | 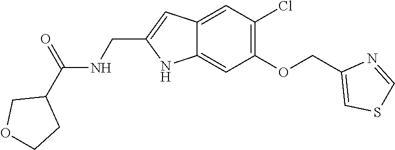 [SMILES: O=C(NCc1cc2cc(Cl)c(OCc3cscn3)cc2[nH]1)C1CCOC1] | N-((5-chloro-6-(thiazol-4- ylmethoxy)-1H-indol-2- yl)methyl)tetrahydrofuran-3- carboxamide | 391.08 | 392.2 |
| 268 | 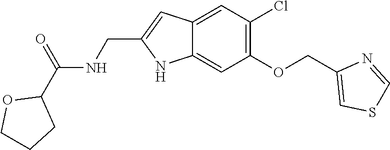 [SMILES: O=C(NCc1cc2cc(Cl)c(OCc3cscn3)cc2[nH]1)C1CCCO1] | N-((5-chloro-6-(thiazol-4- ylmethoxy)-1H-indol-2- yl)methyl)tetrahydrofuran-2- carboxamide | 391.08 | 392.1 |
| 269 | 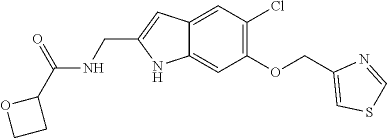 [SMILES: O=C(NCc1cc2cc(Cl)c(OCc3cscn3)cc2[nH]1)C1CCO1] | N-((5-chloro-6-(thiazol-4- ylmethoxy)-1H-indol-2- yl)methyl)oxetane-2- carboxamide | 377.06 | 378.0 |
| 270 | 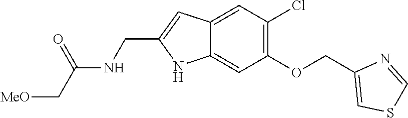 [SMILES: COCC(=O)NCc1cc2cc(Cl)c(OCc3cscn3)cc2[nH]1] | N-((5-chloro-6-(thiazol-4- ylmethoxy)-1H-indol-2- yl)methyl)-2- methoxyacetamide | 365.06 | 366.2 |
| 271 | 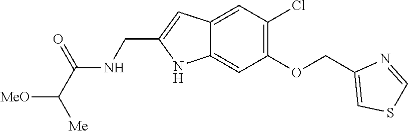 [SMILES: COC(C)C(=O)NCc1cc2cc(Cl)c(OCc3cscn3)cc2[nH]1] | N-((5-chloro-6-(thiazol-4- ylmethoxy)-1H-indol-2- yl)methyl)-2- methoxypropanamide | 379.08 | 380.2 |
| 482 | 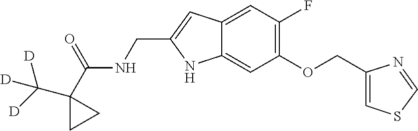 [SMILES: [2H]C([2H])([2H])C1(C(=O)NCc2cc3cc(F)c(OCc4cscn4)cc3[nH]2)CC1] | N-((5-fluoro-6-(thiazol-4- ylmethoxy)-1H-indol-2- yl)methyl)-1-(methyl- d3)cyclopropane-1- carboxamide | 362.13 | 363.3 |
| 483 | 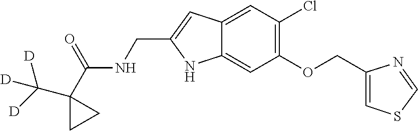 [SMILES: [2H]C([2H])([2H])C1(C(=O)NCc2cc3cc(Cl)c(OCc4cscn4)cc3[nH]2)CC1] | N-((5-chloro-6-(thiazol-4- ylmethoxy)-1H-indol-2- yl)methyl)-1-(methyl- d3)cyclopropane-1- carboxamide | 378.10 | 379.2 |
| 484 | 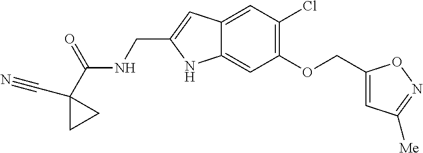 [SMILES: Cc1cc(COc2cc3[nH]c(CNC(=O)C4(C#N)CC4)cc3cc2Cl)on1] | N-((5-chloro-6-((3- methylisoxazol-5-yl)methoxy)- 1H-indol-2-yl)methyl)-1- cyanocyclopropane-1- carboxamide | 384.10 | 385.3 |
| 485 | 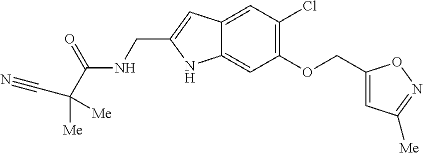 [SMILES: Cc1cc(COc2cc3[nH]c(CNC(=O)C(C)(C)C#N)cc3cc2Cl)on1] | N-((5-chloro-6-((3- methylisoxazol-5-yl)methoxy)- 1H-indol-2-yl)methyl)-2- cyano-2-methylpropanamide | 386.11 | 387.2 |
| 486 | 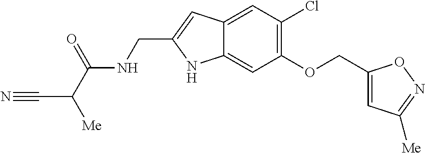 [SMILES: Cc1cc(COc2cc3[nH]c(CNC(=O)C(C)C#N)cc3cc2Cl)on1] | N-((5-chloro-6-((3- methylisoxazol-5-yl)methoxy)- 1H-indol-2-yl)methyl)-2- cyanopropanamide | 372.10 | 373.1 |
| 487 | 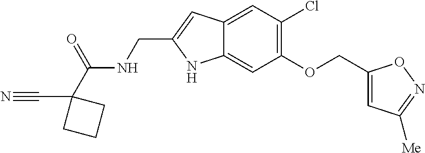 [SMILES: Cc1cc(COc2cc3[nH]c(CNC(=O)C4(C#N)CCC4)cc3cc2Cl)on1] | N-((5-chloro-6-((3- methylisoxazol-5-yl)methoxy)- 1H-indol-2-yl)methyl)-1- cyanocyclobutane-1- carboxamide | 398.11 | 399.2 |
| 488 | 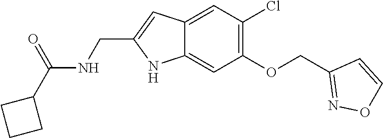 [SMILES: O=C(NCc1cc2cc(Cl)c(OCc3ccon3)cc2[nH]1)C1CCC1] | N-((5-chloro-6-(isoxazol-3- ylmethoxy)-1H-indol-2- yl)methyl) cyclobutanecarboxamide | 359.10 | 360.2 |
| 489 | 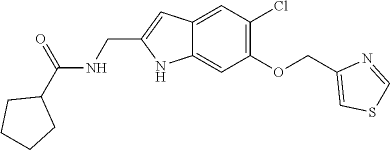 [SMILES: O=C(NCc1cc2cc(Cl)c(OCc3cscn3)cc2[nH]1)C1CCCC1] | N-((5-chloro-6-(thiazol-4- ylmethoxy)-1H-indol-2- yl)methyl) cyclopentanecarboxamide | 389.10 | 390.2 |
| 490 | 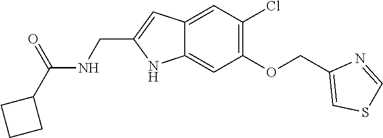 [SMILES: O=C(NCc1cc2cc(Cl)c(OCc3cscn3)cc2[nH]1)C1CCC1] | N-((5-chloro-6-(thiazol-4- ylmethoxy)-1H-indol-2- yl)methyl) cyclobutanecarboxamide | 375.08 | 376.1 |
| 491 | 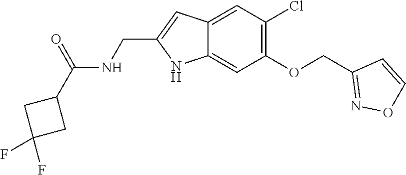 [SMILES: O=C(NCc1cc2cc(Cl)c(OCc3ccon3)cc2[nH]1)C1CC(F)(F)C1] | N-((5-chloro-6-(isoxazol-3- ylmethoxy)-1H-indol-2- yl)methyl)-3,3- difluorocyclobutane-1- carboxamide | 395.08 | 396.2 |
| 492 | 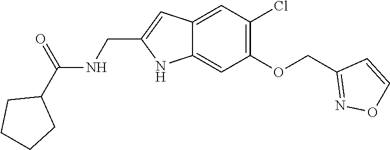 [SMILES: O=C(NCc1cc2cc(Cl)c(OCc3ccon3)cc2[nH]1)C1CCCC1] | N-((5-chloro-6-(isoxazol-3- ylmethoxy)-1H-indol-2- yl)methyl) cyclopentanecarboxamide | 373.12 | 374.2 |
| 493 | 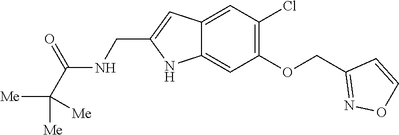 [SMILES: CC(C)(C)C(=O)NCc1cc2cc(Cl)c(OCc3ccon3)cc2[nH]1] | N-((5-chloro-6-(isoxazol-3- ylmethoxy)-1H-indol-2- yl)methyl)pivalamide | 361.12 | 362.2 |
| 494 | 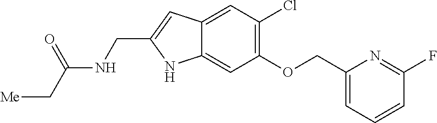 [SMILES: CCC(=O)NCc1cc2cc(Cl)c(OCc3cccc(F)n3)cc2[nH]1] | N-((5-chloro-6-((6- fluoropyridin-2-yl)methoxy)- 1H-indol-2- yl)methyl)propionamide | 361.10 | 362.1 |
| 495 | 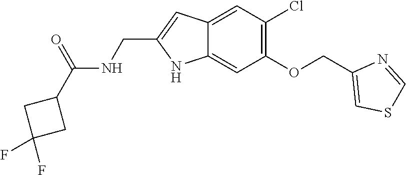 [SMILES: O=C(NCc1cc2cc(Cl)c(OCc3cscn3)cc2[nH]1)C1CC(F)(F)C1] | N-((5-chloro-6-(thiazol-4- ylmethoxy)-1H-indol-2- yl)methyl)-3,3- difluorocyclobutane-1- carboxamide | 411.06 | 412.1 |
| 496 | 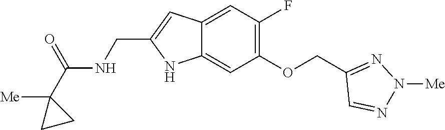 [SMILES: Cn1ncc(COc2cc3[nH]c(CNC(=O)C4(C)CC4)cc3cc2F)n1] | N-((5-fluoro-6-((2-methyl-2H- 1,2,3-triazol-4-yl)methoxy)- 1H-indol-2-yl)methyl)-1- methylcyclopropane-1- carboxamide | 357.16 | 358.2 |
| 497 | 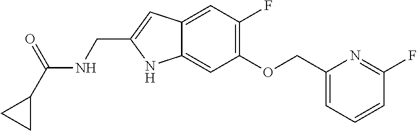 [SMILES: O=C(NCc1cc2cc(F)c(OCc3cccc(F)n3)cc2[nH]1)C1CC1] | N-((5-fluoro-6-((6- fluoropyridin-2-yl)methoxy)- 1H-indol-2- yl)methyl) cyclopropanecarboxamide | 357.13 | 358.3 |
| 498 | 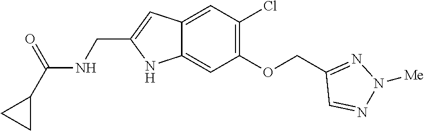 [SMILES: Cn1ncc(COc2cc3[nH]c(CNC(=O)C4CC4)cc3cc2Cl)n1] | N-((5-chloro-6-((2-methyl-2H- 1,2,3-triazol-4-yl)methoxy)- 1H-indol-2- yl)methyl) cyclopropanecarboxamide | 359.11 | 360.1 |
| 499 | 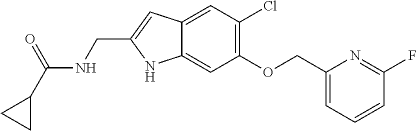 [SMILES: O=C(NCc1cc2cc(Cl)c(OCc3cccc(F)n3)cc2[nH]1)C1CC1] | N-((5-chloro-6-((6- fluoropyridin-2-yl)methoxy)- 1H-indol-2- yl)methyl) cyclopropanecarboxamide | 373.10 | 374.2 |
| 500 | 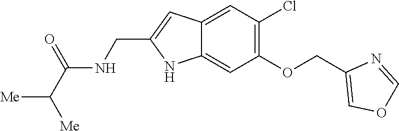 [SMILES: CC(C)C(=O)NCc1cc2cc(Cl)c(OCc3cocn3)cc2[nH]1] | N-((5-chloro-6-(oxazol-4- ylmethoxy)-1H-indol-2- yl)methyl)isobutyramide | 347.10 | 348.3 |
| 501 | 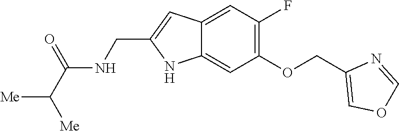 [SMILES: CC(C)C(=O)NCc1cc2cc(F)c(OCc3cocn3)cc2[nH]1] | N-((5-fluoro-6-(oxazol-4- ylmethoxy)-1H-indol-2- yl)methyl)isobutyramide | 331.13 | 332.1 |
| 502 | 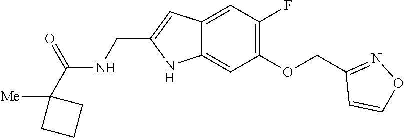 [SMILES: CC1(C(=O)NCc2cc3cc(F)c(OCc4ccon4)cc3[nH]2)CCC1] | N-((5-fluoro-6-(isoxazol-3- ylmethoxy)-1H-indol-2- yl)methyl)-1- methylcyclobutane-1- carboxamide | 357.15 | 358.3 |
| 503 | 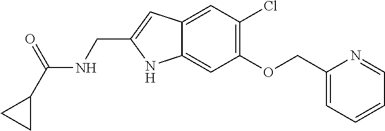 [SMILES: O=C(NCc1cc2cc(Cl)c(OCc3ccccn3)cc2[nH]1)C1CC1] | N-((5-chloro-6-(pyridin-2- ylmethoxy)-1H-indol-2- yl)methyl) cyclopropanecarboxamide | 355.11 | 356.3 |
| 504 | 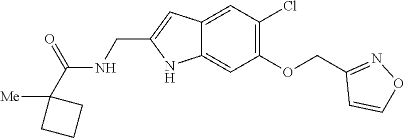 [SMILES: CC1(C(=O)NCc2cc3cc(Cl)c(OCc4ccon4)cc3[nH]2)CCC1] | N-((5-chloro-6-(isoxazol-3- ylmethoxy)-1H-indol-2- yl)methyl)-1- methylcyclobutane-1- carboxamide | 373.12 | 374.3 |
| 505 | 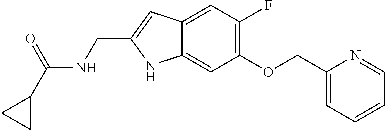 [SMILES: O=C(NCc1cc2cc(F)c(OCc3ccccn3)cc2[nH]1)C1CC1] | N-((5-fluoro-6-(pyridin-2- ylmethoxy)-1H-indol-2- yl)methyl) cyclopropanecarboxamide | 339.14 | 340.3 |
| 506 | 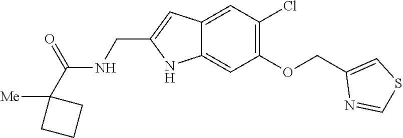 [SMILES: CC1(C(=O)NCc2cc3cc(Cl)c(OCc4cscn4)cc3[nH]2)CCC1] | N-((5-chloro-6-(thiazol-4- ylmethoxy)-1H-indol-2- yl)methyl)-1- methylcyclobutane-1- carboxamide | 389.10 | 390.3 |
| 507 | 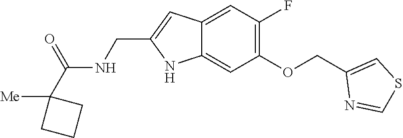 [SMILES: CC1(C(=O)NCc2cc3cc(F)c(OCc4cscn4)cc3[nH]2)CCC1] | N-((5-fluoro-6-(thiazol-4- ylmethoxy)-1H-indol-2- yl)methyl)-1- methylcyclobutane-1- carboxamide | 373.13 | 374.1 |
| 508 | 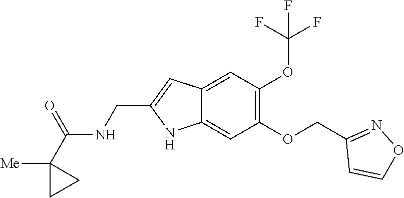 [SMILES: CC1(C(=O)NCc2cc3cc(OC(F)(F)F)c(OCc4ccon4)cc3[nH]2)CC1] | N-((6-(isoxazol-3-ylmethoxy)- 5-(trifluoromethoxy)-1H-indol- 2-yl)methyl)-1- methylcyclopropane-1- carboxamide | 409.12 | 410.2 |
| 509 | 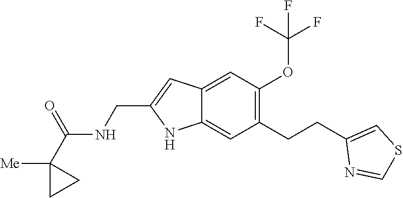 [SMILES: CC1(C(=O)NCc2cc3cc(OC(F)(F)F)c(CCc4cscn4)cc3[nH]2)CC1] | 1-methyl-N-((6-(2-(thiazol-4- yl)ethyl)-5-(trifluoromethoxy)- 1H-indol-2- yl)methyl)cyclopropane-1- carboxamide | 423.12 | 424.1 |
| 510 | 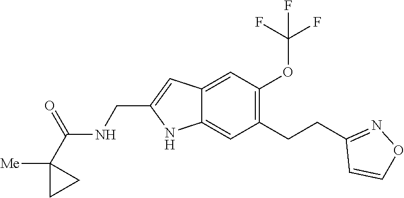 [SMILES: CC1(C(=O)NCc2cc3cc(OC(F)(F)F)c(CCc4ccon4)cc3[nH]2)CC1] | N-((6-(2-(isoxazol-3-yl)ethyl)- 5-(trifluoromethoxy)-1H-indol- 2-yl)methyl)-1- methylcyclopropane-1- carboxamide | 407.15 | 408.1 |
| 511 | 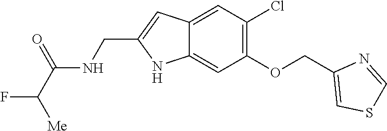 [SMILES: CC(F)C(=O)NCc1cc2cc(Cl)c(OCc3cscn3)cc2[nH]1] | N-((5-chloro-6-(thiazol-4- ylmethoxy)-1H-indol-2- yl)methyl)-2- fluoropropanamide | 367.06 | 368.2 |
| 512 | 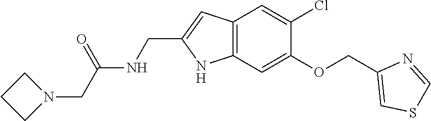 [SMILES: O=C(CN1CCC1)NCc1cc2cc(Cl)c(OCc3cscn3)cc2[nH]1] | 2-(azetidin-1-yl)-N-((5-chloro- 6-(thiazol-4-ylmethoxy)-1H- indol-2-yl)methyl)acetamide | 390.09 | 391.3 |
| 513 | 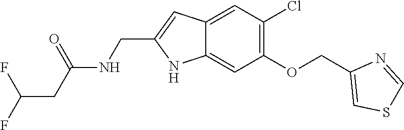 [SMILES: O=C(CC(F)F)NCc1cc2cc(Cl)c(OCc3cscn3)cc2[nH]1] | N-((5-chloro-6-(thiazol-4- ylmethoxy)-1H-indol-2- yl)methyl)-3,3- difluoropropanamide | 385.05 | 386.2 |
| 514 | 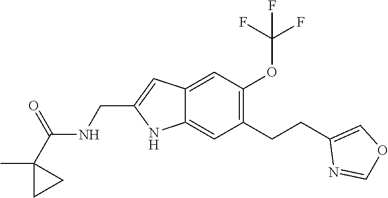 [SMILES: CC1(C(=O)NCc2cc3cc(OC(F)(F)F)c(CCc4cocn4)cc3[nH]2)CC1] | 1-methyl-N-((6-(2-(oxazol-4- yl)ethyl)-5-(trifluoromethoxy)- 1H-indol-2- yl)methyl)cyclopropane-1- carboxamide | 407.15 | 408.1 |
| 515 | 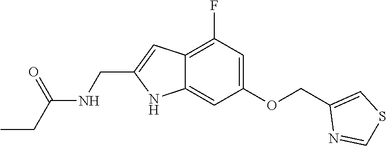 [SMILES: CCC(=O)NCc1cc2c(F)cc(OCc3cscn3)cc2[nH]1] | N-((4-fluoro-6-(thiazol-4- ylmethoxy)-1H-indol-2- yl)methyl)propionamide | 333.09 | 334.1 |
| 516 | 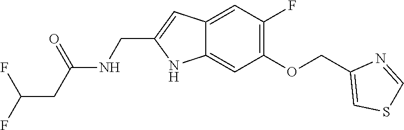 [SMILES: O=C(CC(F)F)NCc1cc2cc(F)c(OCc3cscn3)cc2[nH]1] | 3,3-difluoro-N-((5-fluoro-6- (thiazol-4-ylmethoxy)-1H- indol-2-yl)methyl)propanamide | 369.08 | 370.1 |
| 517 | 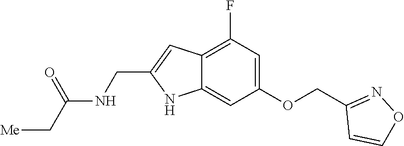 [SMILES: CCC(=O)NCc1cc2c(F)cc(OCc3ccon3)cc2[nH]1] | N-((4-fluoro-6-(isoxazol-3- ylmethoxy)-1H-indol-2- yl)methyl)propionamide | 317.12 | 318.1 |
| 518 | 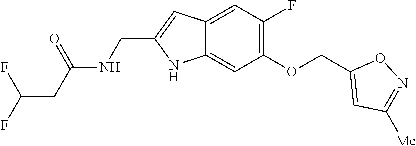 [SMILES: Cc1cc(COc2cc3[nH]c(CNC(=O)CC(F)F)cc3cc2F)on1] | 3,3-difluoro-N-((5-fluoro-6- ((3-methylisoxazol-5- yl)methoxy)-1H-indol-2- yl)methyl)propanamide | 367.11 | 368.1 |
| 519 | 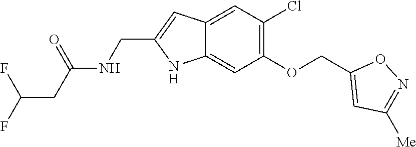 [SMILES: Cc1cc(COc2cc3[nH]c(CNC(=O)CC(F)F)cc3cc2Cl)on1] | N-((5-chloro-6-((3- methylisoxazol-5-yl)methoxy)- 1H-indol-2-yl)methyl)-3,3- difluoropropanamide | 383.08 | 384.1 |
| 520 | 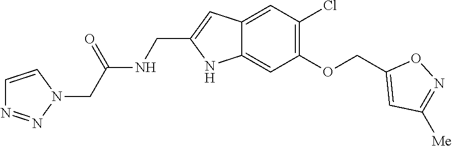 [SMILES: Cc1cc(COc2cc3[nH]c(CNC(=O)Cn4ccnn4)cc3cc2Cl)on1] | N-((5-chloro-6-((3- methylisoxazol-5-yl)methoxy)- 1H-indol-2-yl)methyl)-2-(1H- 1,2,3-triazol-1-yl)acetamide | 400.11 | 401.2 |
| 521 | 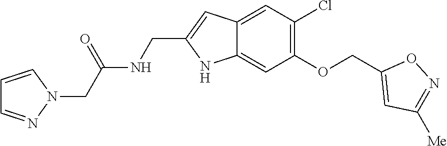 [SMILES: Cc1cc(COc2cc3[nH]c(CNC(=O)Cn4cccn4)cc3cc2Cl)on1] | N-((5-chloro-6-((3- methylisoxazol-5-yl)methoxy)- 1H-indol-2-yl)methyl)-2-(1H- pyrazol-1-yl)acetamide | 399.11 | 400.0 |
| 522 | 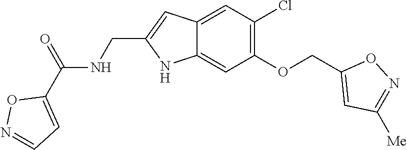 [SMILES: Cc1cc(COc2cc3[nH]c(CNC(=O)c4ccno4)cc3cc2Cl)on1] | N-((5-chloro-6-((3- methylisoxazol-5-yl)methoxy)- 1H-indol-2- yl)methyl)isoxazole-5- carboxamide | 386.08 | 387.1 |
| 523 | 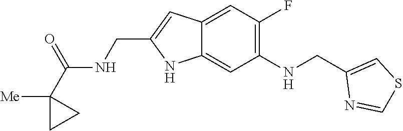 [SMILES: CC1(C(=O)NCc2cc3cc(F)c(NCc4cscn4)cc3[nH]2)CC1] | N-((5-fluoro-6-((thiazol-4- ylmethyl)amino)-1H-indol-2- yl)methyl)-1- methylcyclopropane-1- carboxamide | 358.13 | 359.1 |
| 524 | 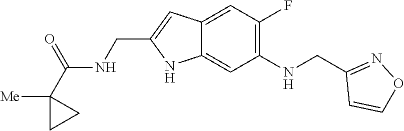 [SMILES: CC1(C(=O)NCc2cc3cc(F)c(NCc4ccon4)cc3[nH]2)CC1] | N-((5-fluoro-6-((isoxazol-3- ylmethyl)amino)-1H-indol-2- yl)methyl)-1- methylcyclopropane-1- carboxamide | 342.15 | 343.1 |
| 525 | 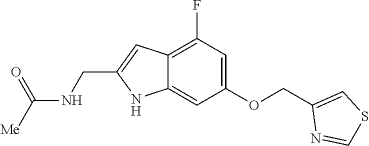 [SMILES: CC(=O)NCc1cc2c(F)cc(OCc3cscn3)cc2[nH]1] | N-((4-fluoro-6-(thiazol-4- ylmethoxy)-1H-indol-2- yl)methyl)acetamide | 319.08 | 320.1 |
| 526 | 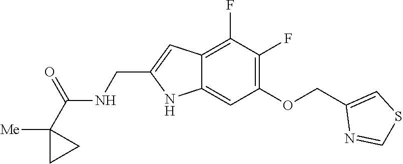 [SMILES: CC1(C(=O)NCc2cc3c(F)c(F)c(OCc4cscn4)cc3[nH]2)CC1] | N-((4,5-difluoro-6-(thiazol-4- ylmethoxy)-1H-indol-2- yl)methyl)-1- methylcyclopropane-1- carboxamide | 377.10 | 378.1 |
| 527 | 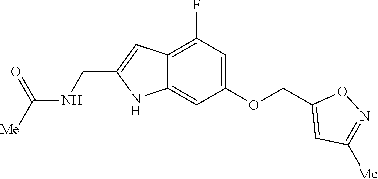 [SMILES: CC(=O)NCc1cc2c(F)cc(OCc3cc(C)no3)cc2[nH]1] | N-((4-fluoro-6-((3- methylisoxazol-5-yl)methoxy)- 1H-indol-2- yl)methyl)acetamide | 317.12 | 318.2 |

### Example 43: (Compound 230)

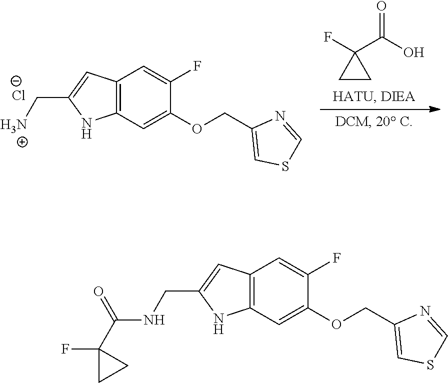 [SMILES: [Cl-] ; [NH3+]Cc1cc2cc(F)c(OCc3cscn3)cc2[nH]1 ; O=C(O)C1(F)CC1 ; O=C(NCc1cc2cc(F)c(OCc3cscn3)cc2[nH]1)C1(F)CC1]

### Step 1: 1-fluoro-N-((5-fluoro-6-(thiazol-4-ylmethoxy)-1H-indol-2-yl)methyl)cyclopropane-1-carboxamide

To a solution of (5-fluoro-6-(thiazol-4-ylmethoxy)-1H-indol-2-yl)methanamine hydrochloride (3.00 g, 9.56 mmol, 1.00 eq) in DCM (30 mL) were added 1-fluorocyclopropanecarboxylic acid (1.19 g, 11.4 mmol, 1.20 eq), DIEA (5.00 mL, 28.6 mmol, 3.00 eq) and HATU (4.73 g, 12.4 mmol, 1.30 eq). The reaction mixture was stirred at 20° C. for 2 h, then was quenched by addition of H₂O (50 mL) and extracted with EtOAc (3×50 mL). The combined organic layers were washed with brine (50 mL), dried over Na₂SO₄, filtered, and concentrated in vacuo. Purification by column chromatography gave a product which was further purified by trituration with EtOAc (5 mL) at 70° C. for 30 min. After cooling to room temperature, the product was filtered and the filter cake was washed with EtOAc (5 mL) and dried under reduced pressure to give 1-fluoro-N-((5-fluoro-6-(thiazol-4-ylmethoxy)-1H-indol-2-yl)methyl)cyclopropane-1-carboxamide (Compound 230). LC-MS (ESI) m/z calcd for C₁₇H₁₅F₂N₃O₂S: 363.09; found: 364.2 [M+H]⁺. ¹H NMR (400 MHz, DMSO-d₆) δ 10.79 (s, 1H), 9.14 (d, J=1.6 Hz, 1H), 8.83 (t, J=5.6 Hz, 1H), 7.78 (d, J=1.2 Hz, 1H), 7.25 (d, J=12.0 Hz, 1H), 7.17 (d, J=7.6 Hz, 1H), 6.17 (s, 1H), 5.25 (s, 2H), 4.44 (d, J=6.0 Hz, 2H), 1.36-1.25 (m, 2H), 1.23-1.17 (m, 2H).

The following compounds in Table T-23 were synthesized using procedures similar to Compound 230 using the appropriate starting materials.

TABLE T-23

| | | | Exact | LCMS, |
|---|---|---|---|---|
| Compound | | | mass | found |
| No. | Structure | IUPAC name | (g/mol) | [M + H]⁺ |
| 231 | 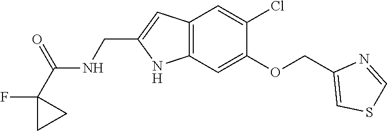 [SMILES: O=C(NCc1cc2cc(Cl)c(OCc3cscn3)cc2[nH]1)C1(F)CC1] | N-((5-chloro-6-(thiazol- 4-ylmethoxy)-1H-indol- 2-yl)methyl)-1- fluorocyclopropane-1- carboxamide | 379.06 | 380.2 |
| 232 | 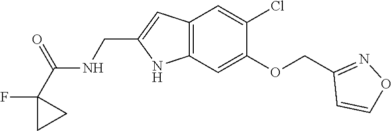 [SMILES: O=C(NCc1cc2cc(Cl)c(OCc3ccon3)cc2[nH]1)C1(F)CC1] | N-((5-chloro-6- (isoxazol-3-ylmethoxy)- 1H-indol-2-yl)methyl)- 1-fluorocyclopropane-1- carboxamide | 363.08 | 364.2 |
| 233 | 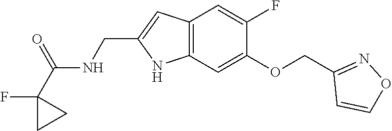 [SMILES: O=C(NCc1cc2cc(F)c(OCc3ccon3)cc2[nH]1)C1(F)CC1] | 1-fluoro-N-((5-fluoro-6- (isoxazol-3-ylmethoxy)- 1H-indol-2- yl)methyl)cyclopropane- 1-carboxamide | 347.11 | 348.2 |

### Example 44: (Compound 234)

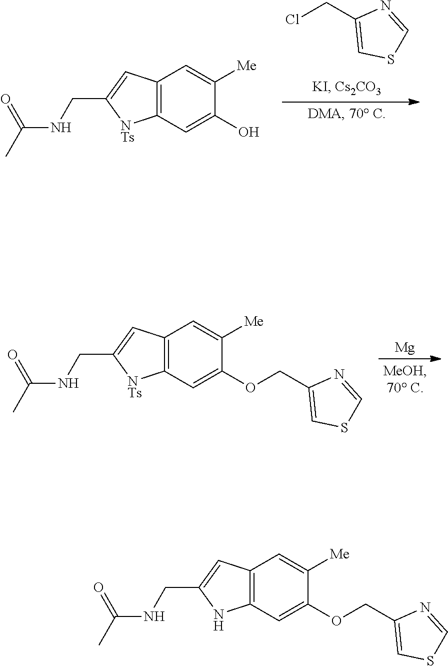

### Step 1: N-((5-methyl-6-(thiazol-4-ylmethoxy)-1-tosyl-1H-indol-2-yl)methyl)acetamide

To a solution of N-((6-hydroxy-5-methyl-1-tosyl-1H-indol-2-yl)methyl)acetamide (155 mg, 416 μmol, 1.00 eq) and 4-(chloromethyl)thiazole hydrochloride (106 mg, 624 μmol, 1.5 eq) in DMA (2 mL) were added KI (13.8 mg, 83.2 μmol, 0.20 eq) and Cs₂CO₃ (271 mg, 832 μmol, 2.00 eq). The reaction mixture was heated to 70° C. and stirred for 2 h under N₂. The mixture was allowed to cool to room temperature, then was quenched by addition of H₂O (10 mL) and extracted with ethyl acetate (3×20 mL). The combined organic layers were washed with brine (2×10 mL), dried over Na₂SO₄, filtered, and concentrated under reduced pressure. Purification by prep-TLC gave N-((5-methyl-6-(thiazol-4-ylmethoxy)-1-tosyl-1H-indol-2-yl)methyl)acetamide. LC-MS (ESI) m/z calcd for C₂₃H₂₃N₃O₄S₂: 469.11; found: 470.3 [M+H]⁺.

### Step 2: N-((5-methyl-6-(thiazol-4-ylmethoxy)-1H-indol-2-yl)methyl)acetamide

To a solution of N-((5-methyl-6-(thiazol-4-ylmethoxy)-1-tosyl-1H-indol-2-yl)methyl) acetamide (120 mg, 255 μmol, 1.00 eq) in MeOH (3 mL) was added Mg (120 mg, 4.94 mmol, 19.3 eq) in one portion under N₂. The mixture was degassed and charged with nitrogen three times. The reaction mixture was then heated to 70° C. and stirred for 6 h under N₂. The mixture was allowed to cool to room temperature, then was adjusted to pH 5 by addition of 1 M aq. HCl and stirred for 5 min. The mixture was then adjusted to pH 8 by addition of saturated aq. Na₂CO₃ solution and stirred for 5 min. The aqueous phase was extracted with dichloromethane (3×10 mL), and the combined organic layers were washed with brine (20 mL), dried over anhydrous Na₂SO₄, filtered, and concentrated under reduced pressure. Purification by prep-HPLC gave N-((5-methyl-6-(thiazol-4-ylmethoxy)1H-indol-2-yl)methyl)acetamide (Compound 234). LC-MS (ESI) m/z calcd for C₁₆H₁₇N₃O₂S: 315.10; found: 316.2 [M+H]⁺. ¹H NMR (400 MHz, DMSO-d₆) δ 10.58 (s, 1H), 9.14 (d, J=2.0 Hz, 1H), 8.27 (t, J=5.2 Hz, 1H), 7.75 (t, J=12.8 Hz, 1H), 7.18 (d, J=10.4 Hz, 1H), 6.96 (s, 1H), 6.09 (s, 1H), 5.20 (s, 2H), 4.34-4.28 (m, 2H), 2.21 (s, 3H), 1.86 (s, 3H).

The following compounds in Table T-24 were synthesized using procedures similar to Compound 234 using the appropriate starting materials.

TABLE T-24

| | | | Exact | LCMS, |
|---|---|---|---|---|
| Compound | | | mass | found |
| No. | Structure | IUPAC name | (g/mol) | [M + H]⁺ |
| 235 | 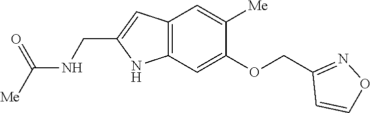 [SMILES: CC(=O)NCc1cc2cc(C)c(OCc3ccon3)cc2[nH]1] | N-((6-(isoxazol-3- ylmethoxy)-5-methyl- 1H-indol-2- yl)methyl)acetamide | 299.13 | 300.3 |
| 528 | 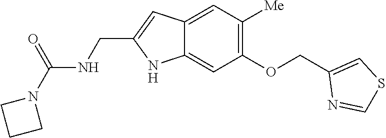 [SMILES: Cc1cc2cc(CNC(=O)N3CCC3)[nH]c2cc1OCc1cscn1] | N-((5-methyl-6- (thiazol-4-ylmethoxy)- 1H-indol-2- yl)methyl)azetidine-1- carboxamide | 356.13 | 357.1 |
| 529 | 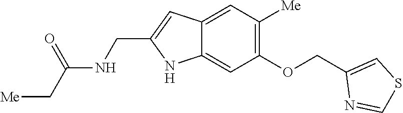 [SMILES: CCC(=O)NCc1cc2cc(C)c(OCc3cscn3)cc2[nH]1] | N-((5-methyl-6- (thiazol-4-ylmethoxy)- 1H-indol-2- yl)methyl)propionamide | 329.12 | 330.1 |
| 530 | 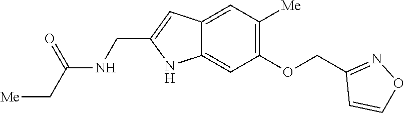 [SMILES: CCC(=O)NCc1cc2cc(C)c(OCc3ccon3)cc2[nH]1] | N-((6-(isoxazol-3- ylmethoxy)-5-methyl- 1H-indol-2- yl)methyl)propionamide | 313.14 | 314.2 |
| 531 | 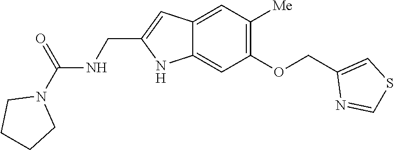 [SMILES: Cc1cc2cc(CNC(=O)N3CCCC3)[nH]c2cc1OCc1cscn1] | N-((5-methyl-6- (thiazol-4-ylmethoxy)- 1H-indol-2- yl)methyl)pyrrolidine- 1-carboxamide | 370.15 | 371.3 |
| 532 | 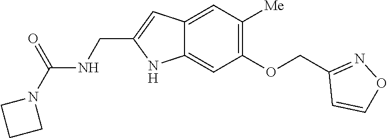 [SMILES: Cc1cc2cc(CNC(=O)N3CCC3)[nH]c2cc1OCc1ccon1] | N-((6-(isoxazol-3- ylmethoxy)-5-methyl- 1H-indol-2- yl)methyl)azetidine-1- carboxamide | 340.15 | 341.2 |
| 533 | 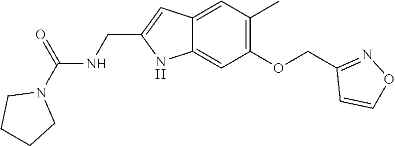 [SMILES: Cc1cc2cc(CNC(=O)N3CCCC3)[nH]c2cc1OCc1ccon1] | N-((6-(isoxazol-3- ylmethoxy)-5-methyl- 1H-indol-2- yl)methyl)pyrrolidine- 1-carboxamide | 354.17 | 355.2 |

### Example 45: (Compound 236)

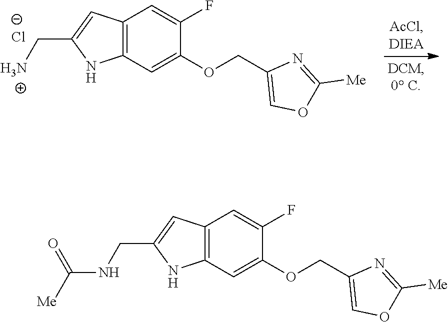 [SMILES: [Cl-] ; Cc1nc(COc2cc3[nH]c(C[NH3+])cc3cc2F)co1 ; CC(=O)NCc1cc2cc(F)c(OCc3coc(C)n3)cc2[nH]1]

### Step 1: N-((5-fluoro-6-((2-methyloxazol-4-yl)methoxy)-1H-indol-2-yl)methyl)acetamide

To a solution of (5-fluoro-6-((2-methyloxazol-4-yl)methoxy)-1H-indol-2-yl)methanamine hydrochloride (160 mg, 0.416 mmol, 1.0 equiv) and DIEA (0.36 mL, 2.1 mmol, 5.0 equiv) in DCM (0.8 mL) at 0° C. was added acetyl chloride (32.6 μL, 0.458 mmol, 1.1 equiv). The resulting mixture was stirred for 2 h then concentrated under reduced pressure. Purification by prep-HPLC gave N-((5-fluoro-6-((2-methyloxazol-4-yl)methoxy)-1H-indol-2-yl)methyl)acetamide (Compound 236). LC-MS (ESI) m/z calcd for C₁₆H₁₆FN₃O₃: 317.12; found: 318.2 [M+H]⁺. ¹H NMR (500 MHz, CD₃CN) 6 9.29 (s, 1H), 7.74 (s, 1H), 7.19 (d, J=12.0 Hz, 1H), 7.13 (d, J=7.3 Hz, 1H), 6.87 (s, 1H), 6.23-6.18 (m, 1H), 4.96 (s, 2H), 4.37 (d, J=5.9 Hz, 2H), 2.41 (s, 3H), 1.91 (s, 3H).

### Example 46: (Compound 272)

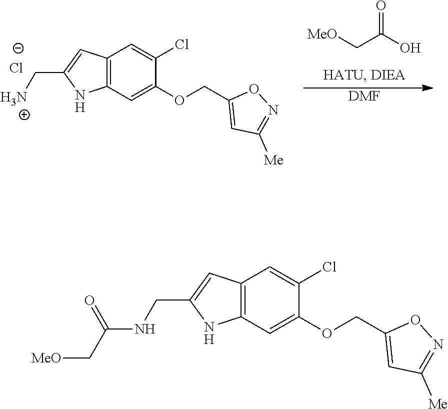

### Step 1: N-((5-chloro-6-((3-methylisoxazol-5-yl)methoxy)-1H-indol-2-yl)methyl)-2-methoxyacetamide

To a solution of 2-methoxyacetic acid (305 μmol, 23.4 μL, 1.00 eq) in DMF (1 mL) was added HATU (174 mg, 457 μmol, 1.50 eq). The mixture was stirred at 20° C. for 0.5 h, then DIEA (212 μL, 1.22 mmol, 4.00 eq) and (5-chloro-6-((3-methylisoxazol-5-yl)methoxy)-1H-indol-2-yl)methanamine hydrochloride (100 mg, 305 μmol, 1.00 eq, HCl salt) were added. The resulting mixture was stirred at 20° C. for 1 h, then was quenched by addition of H₂O (1 mL) and extracted with EtOAc (3×5 mL). The combined organic layers were washed with brine (5 mL), dried over Na₂SO₄, filtered, and concentrated under reduced pressure. Purification by prep-HPLC gave N-((5-chloro-6-((3-methylisoxazol-5-yl)methoxy)-1H-indol-2-yl)methyl)-2-methoxyacetamide (Compound 272). LC-MS (ESI) m/z calcd for C₁₇H₁₈ClN₃O₄: 363.10; found 364.1 [M+H]. ¹H NMR (400 MHz, CDCl₃) δ 9.01 (s, 1H), 7.55 (s, 1H), 7.13 (t, J=4.4 Hz, 1H), 6.93 (s, 1H), 6.24 (d, J=4.4 Hz, 2H), 5.19 (s, 2H), 4.50 (d, J=6.0 Hz, 2H), 3.96 (s, 2H), 3.41 (s, 3H), 2.31 (s, 3H).

The following compounds in Table T-25 were synthesized using procedures similar to Compound 272 using the appropriate starting materials.

TABLE T-25

| | | | Exact | LCMS, |
|---|---|---|---|---|
| Compound | | | mass | found |
| No. | Structure | IUPAC name | (g/mol) | [M + H]⁺ |
| 273 | 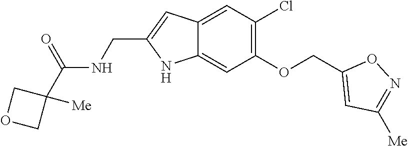 [SMILES: Cc1cc(COc2cc3[nH]c(CNC(=O)C4(C)COC4)cc3cc2Cl)on1] | N-((5-chloro-6-((3- methylisoxazol-5-yl)methoxy)- 1H-indol-2-yl)methyl)-3- methyloxetane-3-carboxamide | 389.11 | 390.2 |
| 274 | 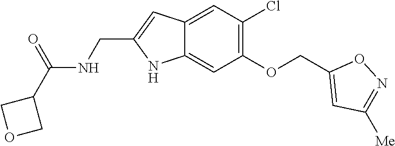 [SMILES: Cc1cc(COc2cc3[nH]c(CNC(=O)C4COC4)cc3cc2Cl)on1] | N-((5-chloro-6-((3- methylisoxazol-5-yl)methoxy)- 1H-indol-2-yl)methyl)oxetane- 3-carboxamide | 375.10 | 376.0 |
| 275 | 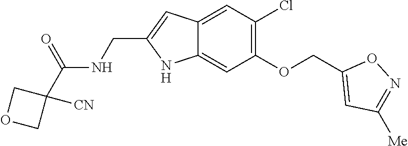 [SMILES: Cc1cc(COc2cc3[nH]c(CNC(=O)C4(C#N)COC4)cc3cc2Cl)on1] | N-((5-chloro-6-((3- methylisoxazol-5-yl)methoxy)- 1H-indol-2-yl)methyl)-3- cyanooxetane-3-carboxamide | 400.09 | 401.2 |
| 276 | 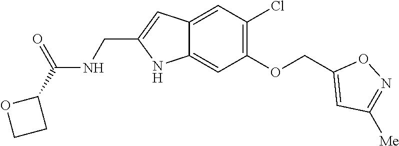 [SMILES: Cc1cc(COc2cc3[nH]c(CNC(=O)[C@@H]4CCO4)cc3cc2Cl)on1] | (S)-N-((5-chloro-6-((3- methylisoxazol-5-yl)methoxy)- 1H-indol-2-yl)methyl)oxetane- 2-carboxamide | 375.10 | 376.2 |
| 277 | 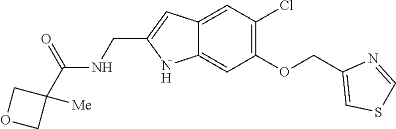 [SMILES: CC1(C(=O)NCc2cc3cc(Cl)c(OCc4cscn4)cc3[nH]2)COC1] | N-((5-chloro-6-(thiazol-4- ylmethoxy)-1H-indol-2- yl)methyl)-3-methyloxetane-3- carboxamide | 391.08 | 392.1 |
| 278 | 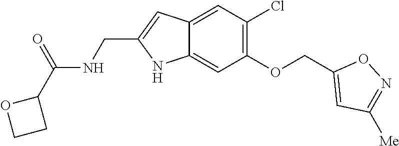 [SMILES: Cc1cc(COc2cc3[nH]c(CNC(=O)C4CCO4)cc3cc2Cl)on1] | N-((5-chloro-6-((3- methylisoxazol-5-yl)methoxy)- 1H-indol-2-yl)methyl)oxetane- 2-carboxamide | 375.10 | 376.1 |
| 279 | 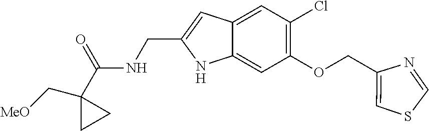 [SMILES: COCC1(C(=O)NCc2cc3cc(Cl)c(OCc4cscn4)cc3[nH]2)CC1] | N-((5-chloro-6-(thiazol-4- ylmethoxy)-1H-indol-2- yl)methyl)-1- (methoxymethyl)cyclopropane- 1-carboxamide | 405.09 | 406.1 |
| 280 | 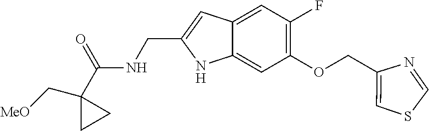 [SMILES: COCC1(C(=O)NCc2cc3cc(F)c(OCc4cscn4)cc3[nH]2)CC1] | N-((5-fluoro-6-(thiazol-4- ylmethoxy)-1H-indol-2- yl)methyl)-1- (methoxymethyl)cyclopropane- 1-carboxamide | 389.12 | 390.3 |
| 534 | 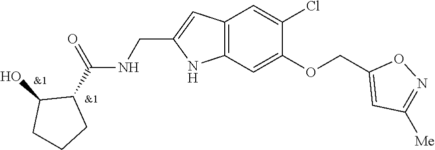 [SMILES: Cc1cc(COc2cc3[nH]c(CNC(=O)[C@@H]4CCC[C@H]4O)cc3cc2Cl)on1] | rac-(1R,2R)-N-((5-chloro-6- ((3-methylisoxazol-5- yl)methoxy)-1H-indol-2- yl)methyl)-2- hydroxycyclopentane-1- carboxamide | 403.13 | 404.3 |

### Example 47: (Compound 281)

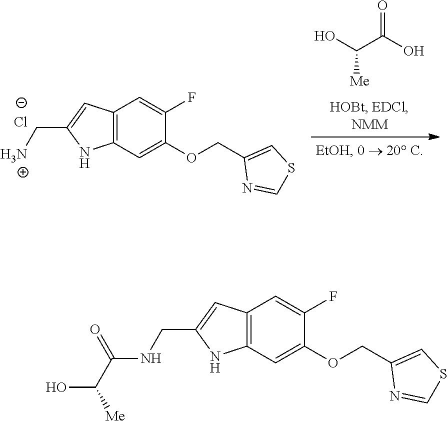 [SMILES: C[C@H](O)C(=O)O ; [Cl-] ; [NH3+]Cc1cc2cc(F)c(OCc3cscn3)cc2[nH]1 ; C[C@H](O)C(=O)NCc1cc2cc(F)c(OCc3cscn3)cc2[nH]1]

### Step 1: (S)—N-((5-fluoro-6-(thiazol-4-ylmethoxy)-1H-indol-2-yl)methyl)-2-hydroxypropanamide

To a solution of (2S)-2-hydroxypropanoic acid (57.4 mg, 637 μmol, 2.00 eq) in EtOH (1.0 mL) were added EDCI (91.6 mg, 478 μmol, 1.50 eq) and HOBt (6.46 mg, 47.8 μmol, 0.15 eq) in one portion at 0° C. The mixture was stirred at 0° C. for 15 min, then (5-fluoro-6-(thiazol-4-ylmethoxy)-1H-indol-2-yl)methanamine hydrochloride (100 mg, 319 μmol, 1.00 eq, HCl salt) and NMM (96.7 mg, 956 μmol, 3.00 eq) were added portionwise. After the addition was complete, the reaction mixture was allowed to warm to 20° C. and stir for 1 h. The mixture was quenched by addition of water (10 mL), extracted with EtOAc (3×10 mL). The combined organic layers were washed with water (3×10 mL), dried over Na₂SO₄, filtered, and concentrated under reduced pressure. Purification by prep-HPLC gave (S)—N-((5-fluoro-6-(thiazol-4-ylmethoxy)-1H-indol-2-yl)methyl)-2-hydroxypropanamide (Compound 281). LC-MS (ESI) m/z calcd for C₁₆H₁₆FN₃O₃S: 349.09; found 350.2 [M+H]⁺. ¹H NMR (400 MHz, DMSO-d₆) δ 10.77 (s, 1H), 9.14 (s, 1H), 8.11 (t, J=6 Hz, 1H), 7.77 (s, 1H), 7.26-7.23 (m, 1H), 7.16 (d, J=7.2 Hz, 1H), 6.14 (s, 1H), 5.25 (s, 2H), 4.42-4.32 (m, 2H), 4.04-3.99 (m, 1H), 1.23 (d, J=6.8 Hz, 3H).

The following compounds in Table T-26 were synthesized using procedures similar to Compound 281 using the appropriate starting materials.

TABLE T-26

| | | | Exact | LCMS, |
|---|---|---|---|---|
| Compound | | | mass | found |
| No. | Structure | IUPAC name | (g/mol) | [M + H]⁺ |
| 282 | 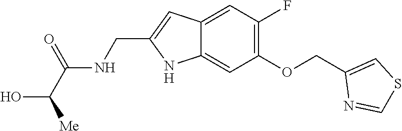 [SMILES: C[C@@H](O)C(=O)NCc1cc2cc(F)c(OCc3cscn3)cc2[nH]1] | (R)-N-((5-fluoro-6- (thiazol-4- ylmethoxy)-1H- indol-2-yl)methyl)- 2- hydroxypropanamide | 349.09 | 350.1 |
| 283 | 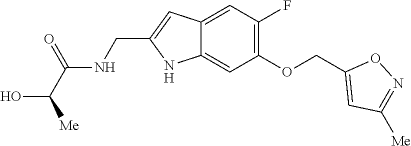 [SMILES: Cc1cc(COc2cc3[nH]c(CNC(=O)[C@@H](C)O)cc3cc2F)on1] | (R)-N-((5-fluoro-6- ((3-methylisoxazol- 5-yl)methoxy)-1H- indol-2-yl)methyl)- 2- hydroxypropanamide | 347.13 | 348.3 |
| 535 | 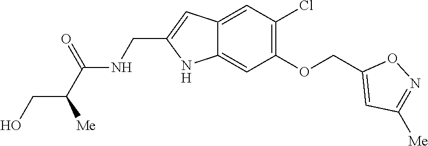 [SMILES: Cc1cc(COc2cc3[nH]c(CNC(=O)[C@@H](C)CO)cc3cc2Cl)on1] | (S)-N-((5-chloro-6- ((3-methylisoxazol- 5-yl)methoxy)-1H- indol-2-yl)methyl)- 3-hydroxy-2- methylpropanamide | 377.11 | 378.1 |
| 536 | 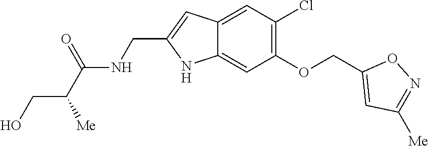 [SMILES: Cc1cc(COc2cc3[nH]c(CNC(=O)[C@H](C)CO)cc3cc2Cl)on1] | (R)-N-((5-chloro-6- ((3-methylisoxazol- 5-yl)methoxy)-1H- indol-2-yl)methyl)- 3-hydroxy-2- methylpropanamide | 377.11 | 378.2 |

### Example 48: (Compound 284)

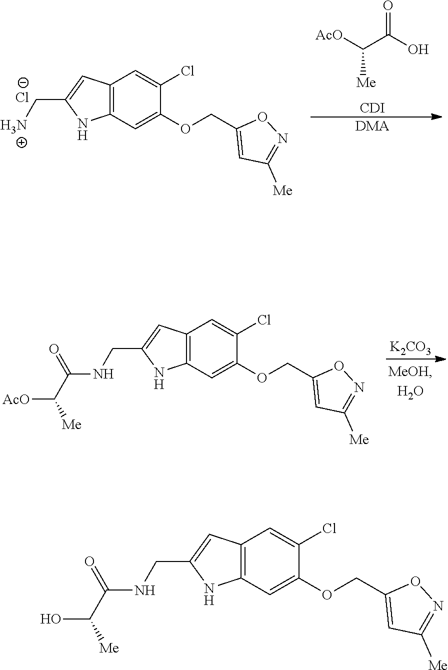

### Step 1: (S)-1-(((5-chloro-6-((3-methylisoxazol-5-yl)methoxy)-1H-indol-2-yl)methyl)amino)-1-oxopropan-2-yl acetate

To a solution of (S)-2-acetoxypropanoic acid (1.45 g, 10.9 mmol, 1.20 eq) in DMA (15 mL) was added CDI (1.78 g, 10.9 mmol, 1.20 eq) in one portion at 20° C. The mixture was stirred at 20° C. for 1 h, then (5-chloro-6-((3-methylisoxazol-5-yl)methoxy)-1H-indol-2-yl)methanamine hydrochloride (3.00 g, 9.14 mmol, 1.00 eq) was added in one portion. The reaction mixture was stirred for 2 h, then was quenched by addition of H₂O (60 mL). After the addition of H₂O, the mixture was stirred at 20° C. for 0.5 h, then filtered. The filter cake was collected and dried under reduced pressure to give (S)-1-(((5-chloro-6-((3-methylisoxazol-5-yl)methoxy)-1H-indol-2-yl)methyl)amino)-1-oxopropan-2-yl acetate, which was used without further purification. LC-MS (ESI) m/z calcd for C₁₉H₂₀ClN₃O₅: 405.11; found 406.2 [M+H]⁺.

### Step 2: (S)—N-((5-chloro-6-((3-methylisoxazol-5-yl)methoxy)-1H-indol-2-yl)methyl)-2-hydroxypropanamide

To a solution of (S)-1-(((5-chloro-6-((3-methylisoxazol-5-yl)methoxy)-1H-indol-2-yl)methyl)amino)-1-oxopropan-2-yl acetate (3.71 g, 9.14 mmol, 1.00 eq) in MeOH (18 mL) and H₂O (6 mL) was added K₂CO₃ (3.16 g, 22.8 mmol, 2.50 eq) in one portion at 20° C. The reaction mixture was stirred at 20° C. for 0.5 h, then was quenched by addition of H₂O (110 mL) and then stirred at 20° C. for an additional 0.5 h. The mixture was then filtered, and the filter cake was collected and dried under reduced pressure. The residue was then triturated with EtOH (18 mL) at 20° C. for 30 mins and isolated by filtration. Further purification by prep-HPLC gave (S)—N-((5-chloro-6-((3-methylisoxazol-5-yl)methoxy)-1H-indol-2-yl)methyl)-2-hydroxypropanamide (Compound 284). LC-MS (ESI) m/z calcd for C₁₇H₁₈ClN₃O₄: 363.10; found 364.0. H NMR (DMSO-d₆, 400 MHz) δ 10.90 (s, 1H), 8.15 (t, J=6.0 Hz, 1H), 7.50 (s, 1H), 7.16 (s, 1H), 6.46 (s, 1H), 6.15 (s, 1H), 5.53 (d, J=4.8 Hz, 1H), 5.27 (s, 2H), 4.42-4.32 (m, 2H), 4.03-3.98 (m, 1H), 2.24 (s, 3H), 1.23 (d, J=6.8 Hz, 3H).

The following compounds in Table T-27 were synthesized using procedures similar to Compound 284 using the appropriate starting materials.

TABLE T-27

| | | | Exact | LCMS, |
|---|---|---|---|---|
| Compound | | | mass | found |
| No. | Structure | IUPAC name | (g/mol) | [M + H]⁺ |
| 285 | 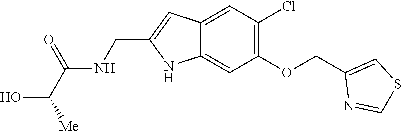 [SMILES: C[C@H](O)C(=O)NCc1cc2cc(Cl)c(OCc3cscn3)cc2[nH]1] | (S)-N-((5-chloro-6- (thiazol-4- ylmethoxy)-1H- indol-2-yl)methyl)- 2- hydroxypropanamide | 365.06 | 366.1 |

### Example 49: (Compound 286)

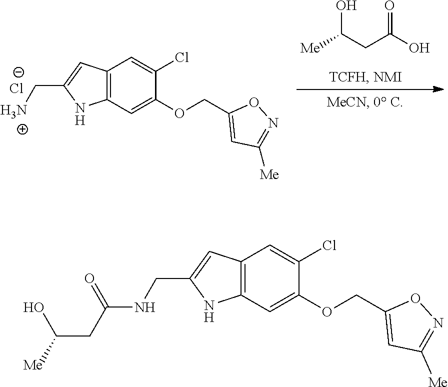 [SMILES: C[C@H](O)CC(=O)O ; [Cl-] ; Cc1cc(COc2cc3[nH]c(C[NH3+])cc3cc2Cl)on1 ; Cc1cc(COc2cc3[nH]c(CNC(=O)C[C@H](C)O)cc3cc2Cl)on1]

### Step 1: (S)—N-((5-chloro-6-((3-methylisoxazol-5-yl)methoxy)-1H-indol-2-yl)methyl)-3-hydroxybutanamide

To a mixture of (5-chloro-6-((3-methylisoxazol-5-yl)methoxy)-1H-indol-2-yl)methanamine hydrochloride (80 mg, 243 μmol, 1.00 eq) and (3S)-3-hydroxybutanoic acid (26.6 mg, 255 μmol, 1.05 eq) in acetonitrile (1 mL) at 0° C. were added 1-methylimidazole (80.0 mg, 975 μmol, 4.00 eq) and N-(chloro(dimethylamino)methylene)-N-methylmethanaminium hexafluorophosphate(V) (102 mg, 365 μmol, 1.50 eq) in portions. The mixture was stirred at 0° C. for 1 h, then was quenched at 0° C. by addition of H₂O (10 mL) and extracted with ethyl acetate (3×5 mL). The combined organic layers were dried over Na₂SO₄, filtered, and concentrated under reduced pressure. Purification by prep-HPLC gave (S)—N-((5-chloro-6-((3-methylisoxazol-5-yl)methoxy)-1H-indol-2-yl)methyl)-3-hydroxybutanamide (Compound 286). LC-MS (ESI) m/z calcd for C₁₈H₂₀ClN₃O₄: 377.11; found 378.2 [M+H]⁺. ¹H NMR (400 MHz, DMSO-d₆) δ 10.91 (s, 1H), 8.29 (t, J=4.8 Hz, 1H), 7.50 (s, 1H), 7.13 (s, 1H), 6.45 (s, 1H), 6.17 (s, 1H), 5.27 (s, 2H), 4.68 (d, J=4.4 Hz, 1H), 4.35 (d, J=5.6 Hz, 2H), 4.04-3.98 (m, 1H), 2.30-2.07 (m, 5H), 1.08 (d, J=6.4 Hz, 3H).

The following compounds in Table T-28 were synthesized using procedures similar to the procedure shown for Compound 286 using the appropriate starting materials.

TABLE T-28

| | | | Exact | LCMS, |
|---|---|---|---|---|
| Compound | | | mass | found |
| No. | Structure | IUPAC name | (g/mol) | [M + H]⁺ |
| 287 | 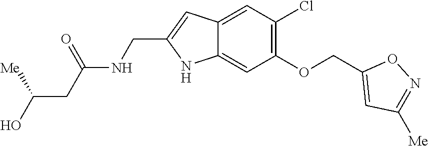 [SMILES: Cc1cc(COc2cc3[nH]c(CNC(=O)C[C@@H](C)O)cc3cc2Cl)on1] | (R)-N-((5-chloro-6-((3- methylisoxazol-5- yl)methoxy)-1H-indol-2- yl)methyl)-3- hydroxybutanamide | 377.11 | 378.1 |
| 288 | 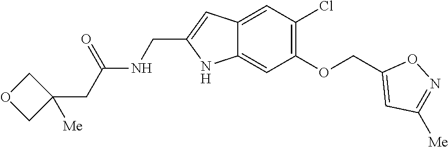 [SMILES: Cc1cc(COc2cc3[nH]c(CNC(=O)CC4(C)COC4)cc3cc2Cl)on1] | N-((5-chloro-6-((3- methylisoxazol-5- yl)methoxy)-1H-indol-2- yl)methyl)-2-(3- methyloxetan-3- yl)acetamide | 403.13 | 404.0 |
| 289 | 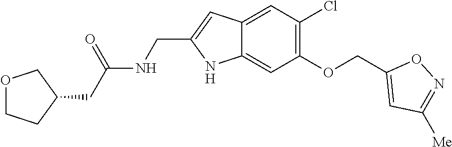 [SMILES: Cc1cc(COc2cc3[nH]c(CNC(=O)C[C@@H]4CCOC4)cc3cc2Cl)on1] | (S)-N-((5-chloro-6-((3- methylisoxazol-5- yl)methoxy)-1H-indol-2- yl)methyl)-2- (tetrahydrofuran-3- yl)acetamide | 403.13 | 404.2 |
| 290 | 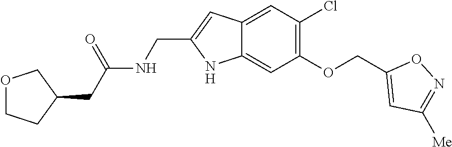 [SMILES: Cc1cc(COc2cc3[nH]c(CNC(=O)C[C@H]4CCOC4)cc3cc2Cl)on1] | (R)-N-((5-chloro-6-((3- methylisoxazol-5- yl)methoxy)-1H-indol-2- yl)methyl)-2- (tetrahydrofuran-3- yl)acetamide | 403.13 | 404.3 |
| 291 | 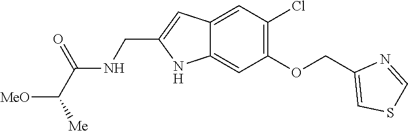 [SMILES: CO[C@@H](C)C(=O)NCc1cc2cc(Cl)c(OCc3cscn3)cc2[nH]1] | (S)-N-((5-chloro-6- (thiazol-4-ylmethoxy)-1H- indol-2-yl)methyl)-2- methoxypropanamide | 379.08 | 380.1 |
| 292 | 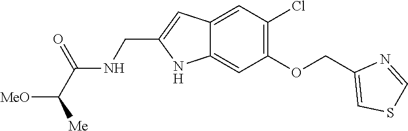 [SMILES: CO[C@H](C)C(=O)NCc1cc2cc(Cl)c(OCc3cscn3)cc2[nH]1] | (R)-N-((5-chloro-6- (thiazol-4-ylmethoxy)-1H- indol-2-yl)methyl)-2- methoxypropanamide | 379.08 | 380.1 |
| 293 | 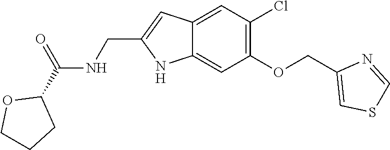 [SMILES: O=C(NCc1cc2cc(Cl)c(OCc3cscn3)cc2[nH]1)[C@@H]1CCCO1] | (S)-N-((5-chloro-6- (thiazol-4-ylmethoxy)-1H- indol-2- yl)methyl)tetrahydrofuran- 2-carboxamide | 391.08 | 392.1 |
| 537 | 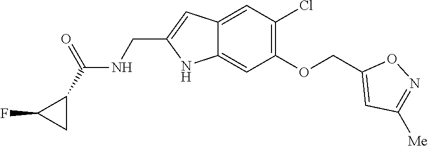 [SMILES: Cc1cc(COc2cc3[nH]c(CNC(=O)[C@@H]4C[C@H]4F)cc3cc2Cl)on1] | (1S,2R)-N-((5-chloro-6- ((3-methylisoxazol-5- yl)methoxy)-1H-indol-2- yl)methyl)-2- fluorocyclopropane-1- carboxamide | 377.09 | 378.0 |
| 538 | 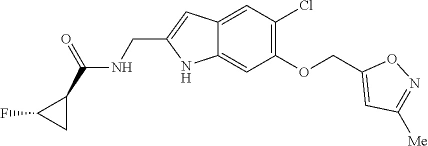 [SMILES: Cc1cc(COc2cc3[nH]c(CNC(=O)[C@H]4C[C@@H]4F)cc3cc2Cl)on1] | (1R,2S)-N-((5-chloro-6- ((3-methylisoxazol-5- yl)methoxy)-1H-indol-2- yl)methyl)-2- fluorocyclopropane-1- carboxamide | 377.09 | 378.2 |
| 539 | 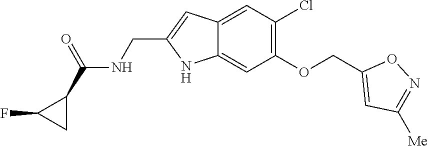 [SMILES: Cc1cc(COc2cc3[nH]c(CNC(=O)[C@H]4C[C@H]4F)cc3cc2Cl)on1] | (1R,2R)-N-((5-chloro-6- ((3-methylisoxazol-5- yl)methoxy)-1H-indol-2- yl)methyl)-2- fluorocyclopropane-1- carboxamide | 377.09 | 378.1 |
| 540 | 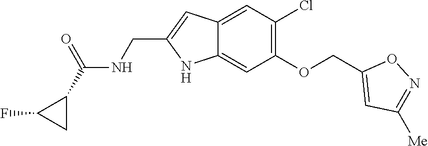 [SMILES: Cc1cc(COc2cc3[nH]c(CNC(=O)[C@@H]4C[C@@H]4F)cc3cc2Cl)on1] | (1S,2S)-N-((5-chloro-6- ((3-methylisoxazol-5- yl)methoxy)-1H-indol-2- yl)methyl)-2- fluorocyclopropane-1- carboxamide | 377.09 | 378.2 |
| 541 | 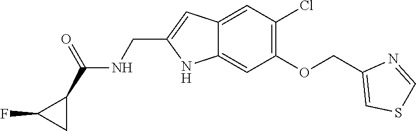 [SMILES: O=C(NCc1cc2cc(Cl)c(OCc3cscn3)cc2[nH]1)[C@H]1C[C@H]1F] | (1R,2R)-N-((5-chloro-6- (thiazol-4-ylmethoxy)-1H- indol-2-yl)methyl)-2- fluorocyclopropane-1- carboxamide | 379.06 | 380.0 |
| 542 | 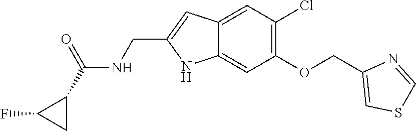 [SMILES: O=C(NCc1cc2cc(Cl)c(OCc3cscn3)cc2[nH]1)[C@@H]1C[C@@H]1F] | (1S,2S)-N-((5-chloro-6- (thiazol-4-ylmethoxy)-1H- indol-2-yl)methyl)-2- fluorocyclopropane-1- carboxamide | 379.06 | 380.2 |
| 543 | 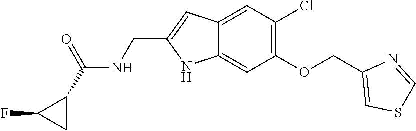 [SMILES: O=C(NCc1cc2cc(Cl)c(OCc3cscn3)cc2[nH]1)[C@@H]1C[C@H]1F] | (1S,2R)-N-((5-chloro-6- (thiazol-4-ylmethoxy)-1H- indol-2-yl)methyl)-2- fluorocyclopropane-1- carboxamide | 379.06 | 380.0 |
| 544 | 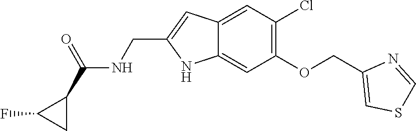 [SMILES: O=C(NCc1cc2cc(Cl)c(OCc3cscn3)cc2[nH]1)[C@H]1C[C@@H]1F] | (1R,2S)-N-((5-chloro-6- (thiazol-4-ylmethoxy)-1H- indol-2-yl)methyl)-2- fluorocyclopropane-1- carboxamide | 379.06 | 380.2 |
| 545 | 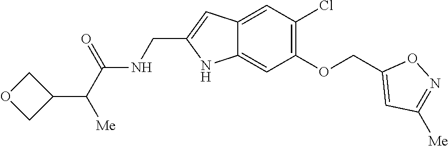 [SMILES: Cc1cc(COc2cc3[nH]c(CNC(=O)C(C)C4COC4)cc3cc2Cl)on1] | N-((5-chloro-6-((3- methylisoxazol-5- yl)methoxy)-1H-indol-2- yl)methyl)-2-(oxetan-3- yl)propanamide | 403.13 | 404.1 |
| 546 | 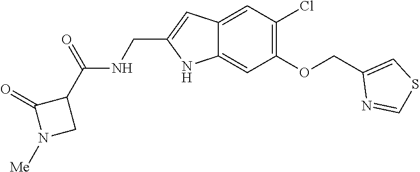 [SMILES: CN1CC(C(=O)NCc2cc3cc(Cl)c(OCc4cscn4)cc3[nH]2)C1=O] | N-((5-chloro-6-(thiazol-4- ylmethoxy)-1H-indol-2- yl)methyl)-1-methyl-2- oxoazetidine-3- carboxamide | 404.07 | 405.1 |

### Example 50: (Compound 294)

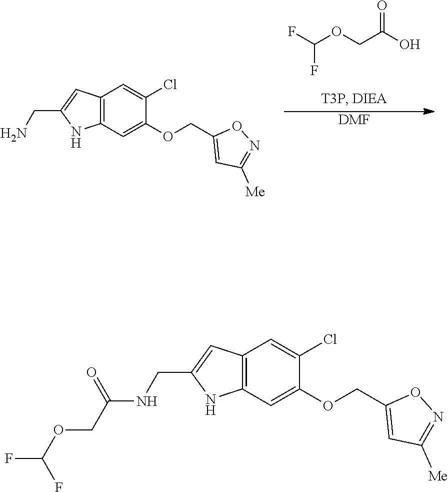

### Step 1: N-((5-chloro-6-((3-methylisoxazol-5-yl)methoxy)-1H-indol-2-yl)methyl)-2-(difluoromethoxy)acetamide

To a solution of (5-chloro-6-((3-methylisoxazol-5-yl)methoxy)-1H-indol-2-yl)methanamine (34.4 mg, 0.118 mmol, 1.0 eq.) and 2-(difluoromethoxy)acetic acid (0.022 g, 0.177 mmol, 1.5 eq.) in DMF (0.6 mL) were added DIEA (0.062 mL, 0.354 mmol, 3 eq.) and T3P (0.098 g, 50 w/w %, 0.153 mmol, 1.3 eq.). The reaction mixture was stirred at 20° C. for 30 min, then was diluted with MeCN (1.5 mL) and H₂O (1.5 mL). Purification by prep HPLC gave N-((5-chloro-6-((3-methylisoxazol-5-yl)methoxy)-1H-indol-2-yl)methyl)-2-(difluoromethoxy)acetamide (Compound 294). LC-MS (ESI) m/z calcd for C₁₇H₁₆ClF₂N₃O₄: 399.08; found 400.1 [M+H]⁺. ¹H NMR (500 MHz, DMSO) δ 10.90 (s, 1H), 8.48 (t, J=5.8 Hz, 1H), 7.45 (s, 1H), 7.08 (s, 1H), 6.70 (t, J=75.1 Hz, 1H), 6.39 (s, 1H), 6.14-6.10 (m, 1H), 5.21 (s, 2H), 4.35 (d, J=5.8 Hz, 2H), 4.29 (s, 2H), 2.17 (s, 3H).

The following compounds in Table T-29 were synthesized using procedures similar to Compound 294 using the appropriate starting materials.

TABLE T-29

| | | | Exact | LCMS, |
|---|---|---|---|---|
| Compound | | | mass | found |
| No. | Structure | IUPAC name | (g/mol) | [M + H]⁺ |
| 295 | 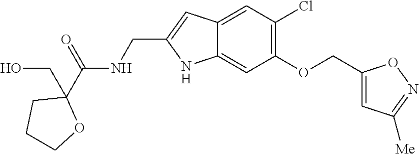 [SMILES: Cc1cc(COc2cc3[nH]c(CNC(=O)C4(CO)CCCO4)cc3cc2Cl)on1] | N-((5-chloro-6-((3-methylisoxazol- 5-yl)methoxy)-1H-indol-2- yl)methyl)-2- (hydroxymethyl)tetrahydrofuran-2- carboxamide | 419.12 | 420.2 |
| 296 | 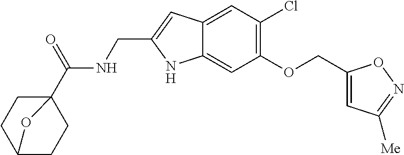 [SMILES: Cc1cc(COc2cc3[nH]c(CNC(=O)C45CCC(CC4)O5)cc3cc2Cl)on1] | N-((5-chloro-6-((3-methylisoxazol- 5-yl)methoxy)-1H-indol-2- yl)methyl)-7- oxabicyclo[2.2.1]heptane-1- carboxamide | 415.13 | 438.1 [M + Na]⁺ |
| 297 | 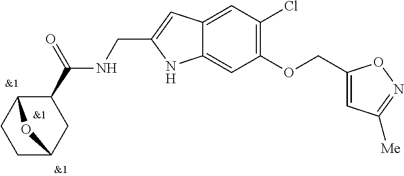 [SMILES: Cc1cc(COc2cc3[nH]c(CNC(=O)[C@H]4C[C@@H]5CC[C@H]4O5)cc3cc2Cl)on1] | (1R,2S,4S)-N-((5-chloro-6-((3- methylisoxazol-5-yl)methoxy)-1H- indol-2-yl)methyl)-7- oxabicyclo[2.2.1]heptane-2- carboxamide and (1S,2R,4R)-N-((5- chloro-6-((3-methylisoxazol-5- yl)methoxy)-1H-indol-2-yl)methyl)- 7-oxabicyclo[2.2.1]heptane-2- carboxamide | 415.13 | 416.2 |
| 298 | 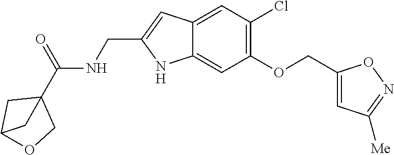 [SMILES: Cc1cc(COc2cc3[nH]c(CNC(=O)C45COC(C4)C5)cc3cc2Cl)on1] | N-((5-chloro-6-((3-methylisoxazol- 5-yl)methoxy)-1H-indol-2- yl)methyl)-2- oxabicyclo[2.1.1]hexane-4- carboxamide | 401.11 | 402.2 |
| 299 | 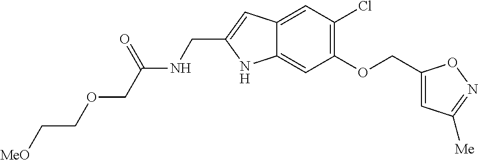 [SMILES: COCCOCC(=O)NCc1cc2cc(Cl)c(OCc3cc(C)no3)cc2[nH]1] | N-((5-chloro-6-((3-methylisoxazol- 5-yl)methoxy)-1H-indol-2- yl)methyl)-2-(2- methoxyethoxy)acetamide | 407.12 | 408.1 |
| 300 | 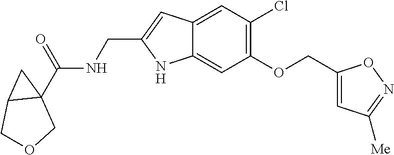 [SMILES: Cc1cc(COc2cc3[nH]c(CNC(=O)C45COCC4C5)cc3cc2Cl)on1] | N-((5-chloro-6-((3-methylisoxazol- 5-yl)methoxy)-1H-indol-2- yl)methyl)-3- oxabicyclo[3.1.0]hexane-1- carboxamide | 401.11 | 402.1 |
| 301 | 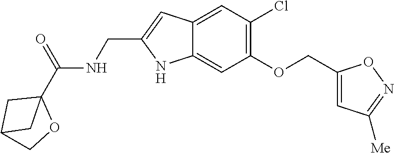 [SMILES: Cc1cc(COc2cc3[nH]c(CNC(=O)C45CC(CO4)C5)cc3cc2Cl)on1] | N-((5-chloro-6-((3-methylisoxazol- 5-yl)methoxy)-1H-indol-2- yl)methyl)-2- oxabicyclo[2.1.1]hexane-1- carboxamide | 401.11 | 402.1 |
| 302 | 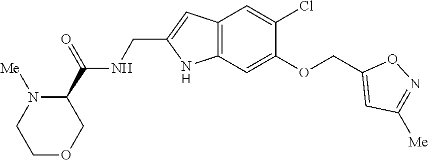 [SMILES: Cc1cc(COc2cc3[nH]c(CNC(=O)[C@H]4COCCN4C)cc3cc2Cl)on1] | (R)-N-((5-chloro-6-((3- methylisoxazol-5-yl)methoxy)-1H- indol-2-yl)methyl)-4- methylmorpholine-3-carboxamide | 418.14 | 419.2 |
| 303 | 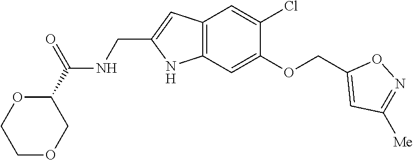 [SMILES: Cc1cc(COc2cc3[nH]c(CNC(=O)[C@@H]4COCCO4)cc3cc2Cl)on1] | (S)-N-((5-chloro-6-((3- methylisoxazol-5-yl)methoxy)-1H- indol-2-yl)methyl)-1,4-dioxane-2- carboxamide | 405.11 | 405.7 |
| 304 | 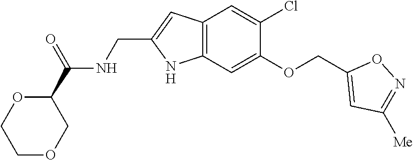 [SMILES: Cc1cc(COc2cc3[nH]c(CNC(=O)[C@H]4COCCO4)cc3cc2Cl)on1] | (R)-N-((5-chloro-6-((3- methylisoxazol-5-yl)methoxy)-1H- indol-2-yl)methyl)-1,4-dioxane-2- carboxamide | 405.11 | 405.8 |
| 305 | 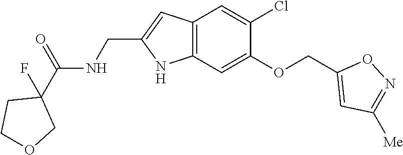 [SMILES: Cc1cc(COc2cc3[nH]c(CNC(=O)C4(F)CCOC4)cc3cc2Cl)on1] | N-((5-chloro-6-((3-methylisoxazol- 5-yl)methoxy)-1H-indol-2- yl)methyl)-3-fluorotetrahydrofuran- 3-carboxamide | 407.10 | 408.2 |
| 306 | 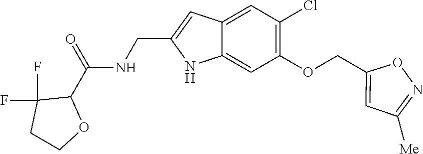 [SMILES: Cc1cc(COc2cc3[nH]c(CNC(=O)C4OCCC4(F)F)cc3cc2Cl)on1] | N-((5-chloro-6-((3-methylisoxazol- 5-yl)methoxy)-1H-indol-2- yl)methyl)-3,3- difluorotetrahydrofuran-2- carboxamide | 425.10 | 426.2 |
| 307 | 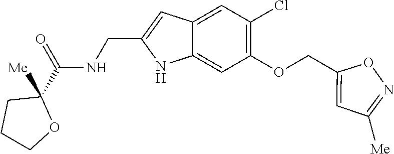 [SMILES: Cc1cc(COc2cc3[nH]c(CNC(=O)[C@@]4(C)CCCO4)cc3cc2Cl)on1] | (R)-N-((5-chloro-6-((3- methylisoxazol-5-yl)methoxy)-1H- indol-2-yl)methyl)-2- methyltetrahydrofuran-2- carboxamide | 403.13 | 404.2 |
| 547 | 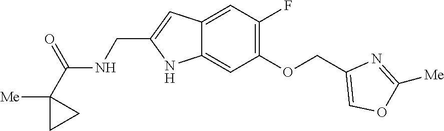 [SMILES: Cc1nc(COc2cc3[nH]c(CNC(=O)C4(C)CC4)cc3cc2F)co1] | N-((5-fluoro-6-((2-methyloxazol-4- yl)methoxy)-1H-indol-2-yl)methyl)- 1-methylcyclopropane-1- carboxamide | 357.15 | 358.0 |
| 548 | 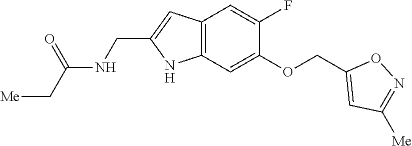 [SMILES: CCC(=O)NCc1cc2cc(F)c(OCc3cc(C)no3)cc2[nH]1] | N-((5-fluoro-6-((3-methylisoxazol- 5-yl)methoxy)-1H-indol-2- yl)methyl)propionamide | 331.13 | 332.5 |
| 549 | 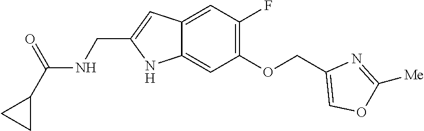 [SMILES: Cc1nc(COc2cc3[nH]c(CNC(=O)C4CC4)cc3cc2F)co1] | N-((5-fluoro-6-((2-methyloxazol-4- yl)methoxy)-1H-indol-2- yl)methyl)cyclopropanecarboxamide | 343.13 | 344.2 |
| 550 | 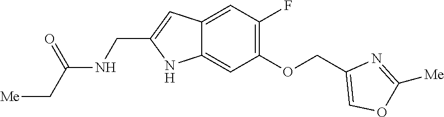 [SMILES: CCC(=O)NCc1cc2cc(F)c(OCc3coc(C)n3)cc2[nH]1] | N-((5-fluoro-6-((2-methyloxazol-4- yl)methoxy)-1H-indol-2- yl)methyl)propionamide | 331.13 | 332.2 |
| 551 | 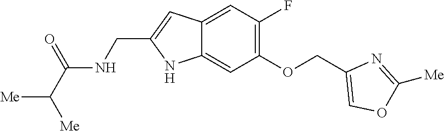 [SMILES: Cc1nc(COc2cc3[nH]c(CNC(=O)C(C)C)cc3cc2F)co1] | N-((5-fluoro-6-((2-methyloxazol-4- yl)methoxy)-1H-indol-2- yl)methyl)isobutyramide | 345.15 | 346.2 |
| 552 | 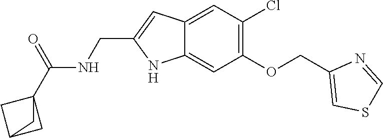 [SMILES: O=C(NCc1cc2cc(Cl)c(OCc3cscn3)cc2[nH]1)C12CC(C1)C2] | N-((5-chloro-6-(thiazol-4- ylmethoxy)-1H-indol-2- yl)methyl)bicyclo[1.1.1]pentane-1- carboxamide | 387.08 | 388.2 |
| 553 | 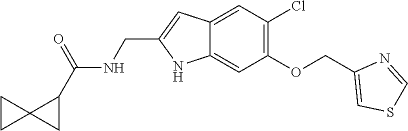 [SMILES: O=C(NCc1cc2cc(Cl)c(OCc3cscn3)cc2[nH]1)C1CC12CC2] | N-((5-chloro-6-(thiazol-4- ylmethoxy)-1H-indol-2- yl)methyl)spiro[2.2]pentane-1- carboxamide | 387.08 | 388.2 |
| 554 | 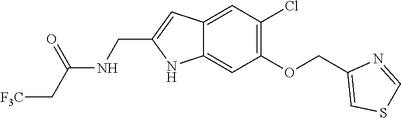 [SMILES: O=C(CC(F)(F)F)NCc1cc2cc(Cl)c(OCc3cscn3)cc2[nH]1] | N-((5-chloro-6-(thiazol-4- ylmethoxy)-1H-indol-2-yl)methyl)- 3,3,3-trifluoropropanamide | 403.04 | 404.1 |
| 555 | 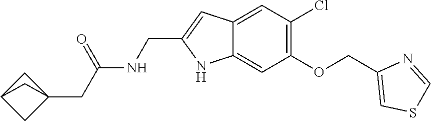 [SMILES: O=C(CC12CC(C1)C2)NCc1cc2cc(Cl)c(OCc3cscn3)cc2[nH]1] | 2-(bicyclo[1.1.1]pentan-1-yl)-N- ((5-chloro-6-(thiazol-4-ylmethoxy)- 1H-indol-2-yl)methyl)acetamide | 401.10 | 402.2 |
| 556 | 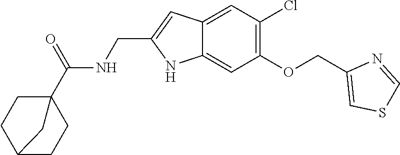 [SMILES: O=C(NCc1cc2cc(Cl)c(OCc3cscn3)cc2[nH]1)C12CCC(CC1)C2] | N-((5-chloro-6-(thiazol-4- ylmethoxy)-1H-indol-2- yl)methyl)bicyclo[2.2.1]heptane-1- carboxamide | 415.11 | 416.2 |
| 557 | 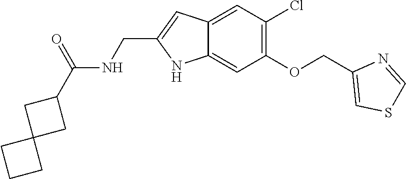 [SMILES: O=C(NCc1cc2cc(Cl)c(OCc3cscn3)cc2[nH]1)C1CC2(CCC2)C1] | N-((5-chloro-6-(thiazol-4- ylmethoxy)-1H-indol-2- yl)methyl)spiro[3.3]heptane-2- carboxamide | 415.11 | 416.2 |
| 558 | 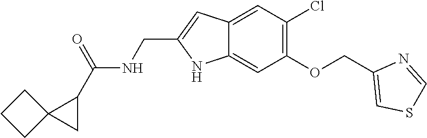 [SMILES: O=C(NCc1cc2cc(Cl)c(OCc3cscn3)cc2[nH]1)C1CC12CCC2] | N-((5-chloro-6-(thiazol-4- ylmethoxy)-1H-indol-2- yl)methyl)spiro[2.3]hexane-1- carboxamide | 401.10 | 402.2 |
| 559 | 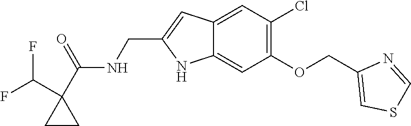 [SMILES: O=C(NCc1cc2cc(Cl)c(OCc3cscn3)cc2[nH]1)C1(C(F)F)CC1] | N-((5-chloro-6-(thiazol-4- ylmethoxy)-1H-indol-2-yl)methyl)- 1-(difluoromethyl)cyclopropane-1- carboxamide | 411.06 | 412.1 |
| 560 | 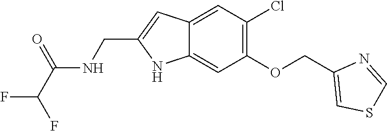 [SMILES: O=C(NCc1cc2cc(Cl)c(OCc3cscn3)cc2[nH]1)C(F)F] | N-((5-chloro-6-(thiazol-4- ylmethoxy)-1H-indol-2-yl)methyl)- 2,2-difluoroacetamide | 371.03 | 372.1 |
| 561 | 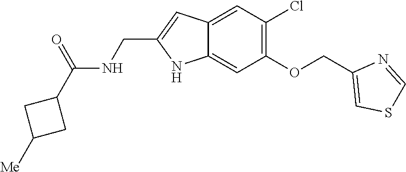 [SMILES: CC1CC(C(=O)NCc2cc3cc(Cl)c(OCc4cscn4)cc3[nH]2)C1] | N-((5-chloro-6-(thiazol-4- ylmethoxy)-1H-indol-2-yl)methyl)- 3-methylcyclobutane-1-carboxamide | 389.10 | 390.2 |
| 562 | 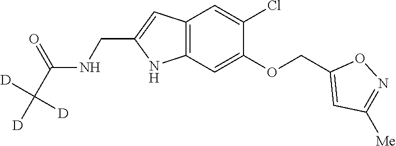 [SMILES: [2H]C([2H])([2H])C(=O)NCc1cc2cc(Cl)c(OCc3cc(C)no3)cc2[nH]1] | N-((5-chloro-6-((3-methylisoxazol- 5-yl)methoxy)-1H-indol-2- yl)methyl)acetamide-2,2,2-d3 | 336.11 | 337.1 |
| 563 | 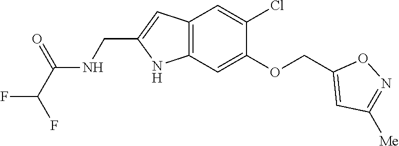 [SMILES: Cc1cc(COc2cc3[nH]c(CNC(=O)C(F)F)cc3cc2Cl)on1] | N-((5-chloro-6-((3-methylisoxazol- 5-yl)methoxy)-1H-indol-2- yl)methyl)-2,2-difluoroacetamide | 369.07 | 370.2 |
| 564 | 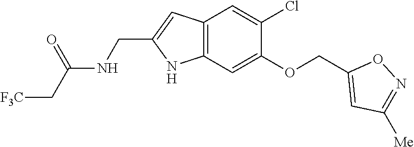 [SMILES: Cc1cc(COc2cc3[nH]c(CNC(=O)CC(F)(F)F)cc3cc2Cl)on1] | N-((5-chloro-6-((3-methylisoxazol- 5-yl)methoxy)-1H-indol-2- yl)methyl)-3,3,3- trifluoropropanamide | 401.08 | 402.2 |
| 565 | 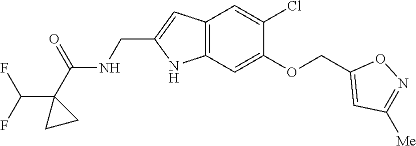 [SMILES: Cc1cc(COc2cc3[nH]c(CNC(=O)C4(C(F)F)CC4)cc3cc2Cl)on1] | N-((5-chloro-6-((3-methylisoxazol- 5-yl)methoxy)-1H-indol-2- yl)methyl)-1- (difluoromethyl)cyclopropane-1- carboxamide | 409.10 | 410.2 |
| 566 | 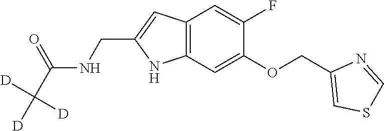 [SMILES: [2H]C([2H])([2H])C(=O)NCc1cc2cc(F)c(OCc3cscn3)cc2[nH]1] | N-((5-fluoro-6-(thiazol-4- ylmethoxy)-1H-indol-2- yl)methyl)acetamide-2,2,2-d3 | 322.10 | 345.1 [M + Na]⁺ |
| 567 | 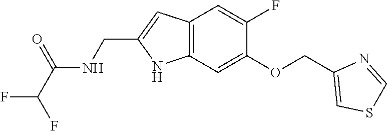 [SMILES: O=C(NCc1cc2cc(F)c(OCc3cscn3)cc2[nH]1)C(F)F] | 2,2-difluoro-N-((5-fluoro-6-(thiazol- 4-ylmethoxy)-1H-indol-2- yl)methyl)acetamide | 355.06 | 356.2 |
| 568 | 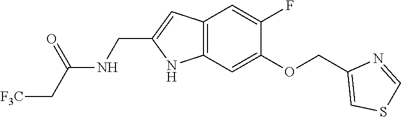 [SMILES: O=C(CC(F)(F)F)NCc1cc2cc(F)c(OCc3cscn3)cc2[nH]1] | 3,3,3-trifluoro-N-((5-fluoro-6- (thiazol-4-ylmethoxy)-1H-indol-2- yl)methyl)propanamide | 387.10 | 388.2 |
| 569 | 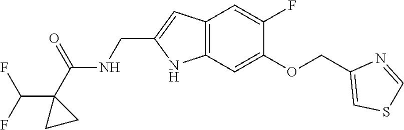 [SMILES: O=C(NCc1cc2cc(F)c(OCc3cscn3)cc2[nH]1)C1(C(F)F)CC1] | 1-(difluoromethyl)-N-((5-fluoro-6- (thiazol-4-ylmethoxy)-1H-indol-2- yl)methyl)cyclopropane-1- carboxamide | 395.09 | 396.2 |
| 570 | 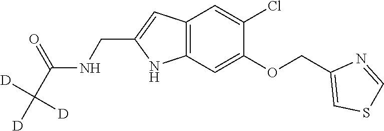 [SMILES: [2H]C([2H])([2H])C(=O)NCc1cc2cc(Cl)c(OCc3cscn3)cc2[nH]1] | N-((5-chloro-6-(thiazol-4- ylmethoxy)-1H-indol-2- yl)methyl)acetamide-2,2,2-d3 | 338.07 | 339.1 |
| 571 | 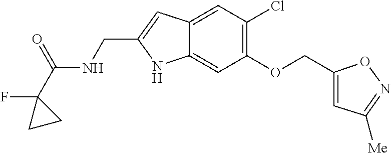 [SMILES: Cc1cc(COc2cc3[nH]c(CNC(=O)C4(F)CC4)cc3cc2Cl)on1] | N-((5-chloro-6-((3-methylisoxazol- 5-yl)methoxy)-1H-indol-2- yl)methyl)-1-fluorocyclopropane-1- carboxamide | 377.09 | 378.2 |
| 572 | 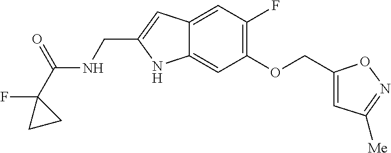 [SMILES: Cc1cc(COc2cc3[nH]c(CNC(=O)C4(F)CC4)cc3cc2F)on1] | 1-fluoro-N-((5-fluoro-6-((3- methylisoxazol-5-yl)methoxy)-1H- indol-2-yl)methyl)cyclopropane-1- carboxamide | 361.13 | 362.2 |
| 573 | 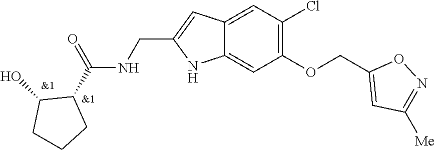 [SMILES: Cc1cc(COc2cc3[nH]c(CNC(=O)[C@@H]4CCC[C@@H]4O)cc3cc2Cl)on1] | rac-(1R,2S)-N-((5-chloro-6-((3- methylisoxazol-5-yl)methoxy)-1H- indol-2-yl)methyl)-2- hydroxycyclopentane-1- carboxamide | 403.13 | 404.1 |

### Example 51: (Compound 308)

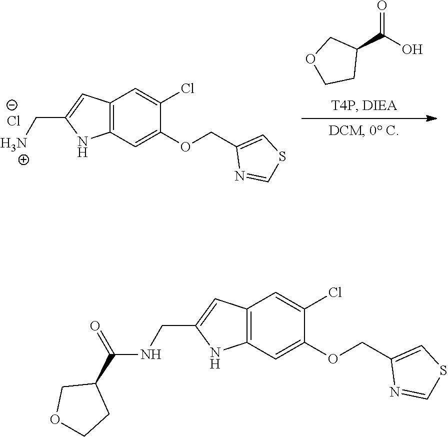 [SMILES: O=C(O)[C@H]1CCOC1 ; [Cl-] ; [NH3+]Cc1cc2cc(Cl)c(OCc3cscn3)cc2[nH]1 ; O=C(NCc1cc2cc(Cl)c(OCc3cscn3)cc2[nH]1)[C@H]1CCOC1]

### Step 1: (S)—N-((5-chloro-6-(thiazol-4-ylmethoxy)-1H-indol-2-yl)methyl)tetrahydrofuran-3-carboxamide

To a mixture of (5-chloro-6-(thiazol-4-ylmethoxy)-1H-indol-2-yl)methanamine hydrochloride (142 mg, 431 μmol, 1.00 eq) and (S)-tetrahydrofuran-3-carboxylic acid (50.0 mg, 431 mol, 1.00 eq) in DCM (4 mL) was added DIEA (167 mg, 1.29 mmol, 3.00 eq) dropwise at 20° C. The resulting mixture was cooled to 0° C. and then 2,4,6-tributyl-1,3,5,2,4,6-trioxatriphosphinane 2,4,6-trioxide (T4P, 372 mg, 517 μmol, 1.20 eq, 50% w/w in EtOAc) was added dropwise to the reaction mixture. The reaction mixture was stirred at 0° C. for 1 h, then was quenched by addition of water (10 mL) at 0° C. and extracted with DCM/isopropanol (3/1) (3×10 mL). The combined organic phase was washed with brine (10 mL), dried with anhydrous Na₂SO₄, filtered, and concentrated under reduced pressure. Purification by prep-HPLC gave (S)—N-((5-chloro-6-(thiazol-4-ylmethoxy)-1H-indol-2-yl)methyl)tetrahydrofuran-3-carboxamide (Compound 308). LC-MS (ESI) m/z calcd for C₁₈H₁₈ClN₃O₃S: 391.08; found 391.9 [M+H]⁺. ¹H NMR (400 MHz, DMSO-d₆) δ 10.92 (s, 1H), 9.15 (d, J=2.0 Hz, 1H), 8.42 (t, J=5.6 Hz, 1H), 7.76 (d, J=1.2 Hz, 1H), 7.51 (s, 1H), 7.17 (s, 1H), 6.17 (s, 1H), 5.27 (s, 2H), 4.36 (d, J=5.6 Hz, 2H), 3.86 (t, J=8.0 Hz, 1H), 3.78-3.71 (m, 1H), 3.69-3.65 (m, 2H), 3.01-2.99 (m, 1H), 2.04-2.00 (m, 2H).

The following compounds in Table T-30 were synthesized using procedures similar to Compound 308 using the appropriate starting materials.

TABLE T-30

| | | | Exact | LCMS, |
|---|---|---|---|---|
| Compound | | | mass | found |
| No. | Structure | IUPAC name | (g/mol) | [M + H]⁺ |
| 309 | 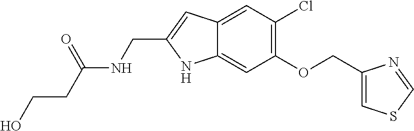 [SMILES: O=C(CCO)NCc1cc2cc(Cl)c(OCc3cscn3)cc2[nH]1] | N-((5-chloro-6-(thiazol-4- ylmethoxy)-1H-indol-2- yl)methyl)-3- hydroxypropanamide | 365.06 | 365.9 |
| 310 | 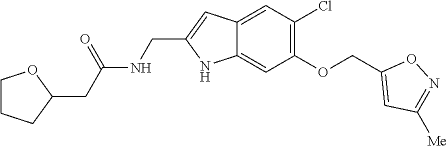 [SMILES: Cc1cc(COc2cc3[nH]c(CNC(=O)CC4CCCO4)cc3cc2Cl)on1] | N-((5-chloro-6-((3- methylisoxazol-5- yl)methoxy)-1H-indol-2- yl)methyl)-2- (tetrahydrofuran-2- yl)acetamide | 403.13 | 404.0 |
| 311 | 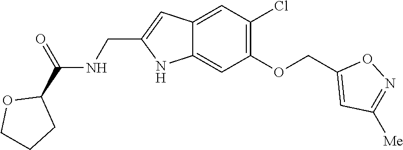 [SMILES: Cc1cc(COc2cc3[nH]c(CNC(=O)[C@H]4CCCO4)cc3cc2Cl)on1] | (R)-N-((5-chloro-6-((3- methylisoxazol-5- yl)methoxy)-1H-indol-2- yl)methyl)tetrahydrofuran- 2-carboxamide | 389.11 | 390.1 |
| 312 | 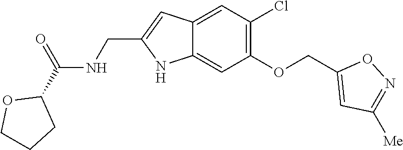 [SMILES: Cc1cc(COc2cc3[nH]c(CNC(=O)[C@@H]4CCCO4)cc3cc2Cl)on1] | (S)-N-((5-chloro-6-((3- methylisoxazol-5- yl)methoxy)-1H-indol-2- yl)methyl)tetrahydrofuran- 2-carboxamide | 389.11 | 390.2 |
| 313 | 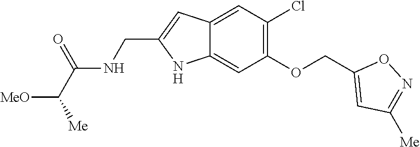 [SMILES: CO[C@@H](C)C(=O)NCc1cc2cc(Cl)c(OCc3cc(C)no3)cc2[nH]1] | (S)-N-((5-chloro-6-((3- methylisoxazol-5- yl)methoxy)-1H-indol-2- yl)methyl)-2- methoxypropanamide | 377.11 | 378.1 |
| 314 | 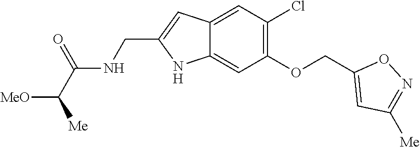 [SMILES: CO[C@H](C)C(=O)NCc1cc2cc(Cl)c(OCc3cc(C)no3)cc2[nH]1] | (R)-N-((5-chloro-6-((3- methylisoxazol-5- yl)methoxy)-1H-indol-2- yl)methyl)-2- methoxypropanamide | 377.11 | 378.1 |
| 315 | 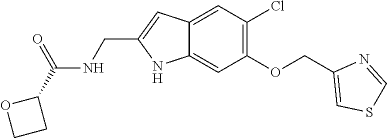 [SMILES: O=C(NCc1cc2cc(Cl)c(OCc3cscn3)cc2[nH]1)[C@@H]1CCO1] | (S)-N-((5-chloro-6- (thiazol-4-ylmethoxy)-1H- indol-2- yl)methyl)oxetane-2- carboxamide | 377.06 | 377.9 |
| 316 | 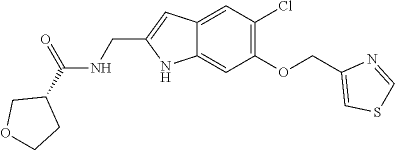 [SMILES: O=C(NCc1cc2cc(Cl)c(OCc3cscn3)cc2[nH]1)[C@@H]1CCOC1] | (R)-N-((5-chloro-6- (thiazol-4-ylmethoxy)-1H- indol-2- yl)methyl)tetrahydrofuran- 3-carboxamide | 391.08 | 391.9 |
| 574 | 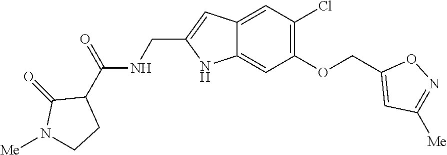 [SMILES: Cc1cc(COc2cc3[nH]c(CNC(=O)C4CCN(C)C4=O)cc3cc2Cl)on1] | N-((5-chloro-6-((3- methylisoxazol-5- yl)methoxy)-1H-indol-2- yl)methyl)-1-methyl-2- oxopyrrolidine-3- carboxamide | 416.13 | 417.1 |
| 575 | 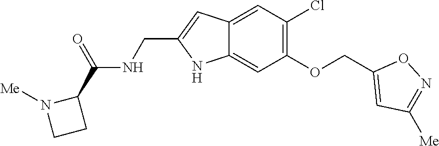 [SMILES: Cc1cc(COc2cc3[nH]c(CNC(=O)[C@H]4CCN4C)cc3cc2Cl)on1] | (R)-N-((5-chloro-6-((3- methylisoxazol-5- yl)methoxy)-1H-indol-2- yl)methyl)-1- methylazetidine-2- carboxamide | 388.13 | 389.2 |
| 576 | 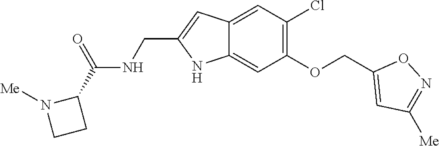 [SMILES: Cc1cc(COc2cc3[nH]c(CNC(=O)[C@@H]4CCN4C)cc3cc2Cl)on1] | (S)-N-((5-chloro-6-((3- methylisoxazol-5- yl)methoxy)-1H-indol-2- yl)methyl)-1- methylazetidine-2- carboxamide | 388.13 | 389.0 |
| 577 | 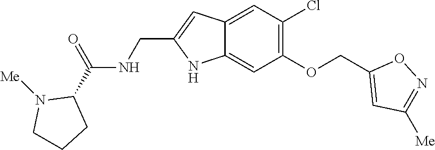 [SMILES: Cc1cc(COc2cc3[nH]c(CNC(=O)[C@@H]4CCCN4C)cc3cc2Cl)on1] | (S)-N-((5-chloro-6-((3- methylisoxazol-5- yl)methoxy)-1H-indol-2- yl)methyl)-1- methylpyrrolidine-2- carboxamide | 402.15 | 403.2 |
| 578 | 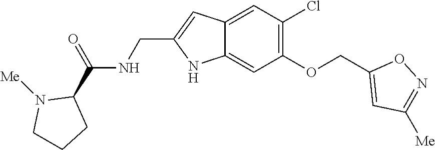 [SMILES: Cc1cc(COc2cc3[nH]c(CNC(=O)[C@H]4CCCN4C)cc3cc2Cl)on1] | (R)-N-((5-chloro-6-((3- methylisoxazol-5- yl)methoxy)-1H-indol-2- yl)methyl)-1- methylpyrrolidine-2- carboxamide | 402.15 | 403.2 |
| 579 | 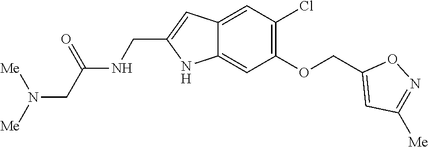 [SMILES: Cc1cc(COc2cc3[nH]c(CNC(=O)CN(C)C)cc3cc2Cl)on1] | N-((5-chloro-6-((3- methylisoxazol-5- yl)methoxy)-1H-indol-2- yl)methyl)-2- (dimethylamino)acetamide | 376.13 | 377.1 |
| 580 | 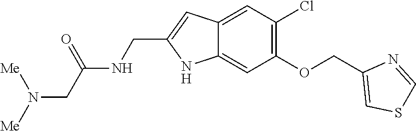 [SMILES: CN(C)CC(=O)NCc1cc2cc(Cl)c(OCc3cscn3)cc2[nH]1] | N-((5-chloro-6-(thiazol-4- ylmethoxy)-1H-indol-2- yl)methyl)-2- (dimethylamino)acetamide | 378.09 | 378.9 |

### Example 52: (Compound 317)

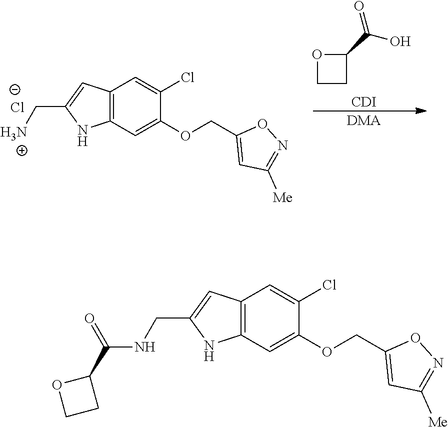

### Step 1: (R)—N-((5-chloro-6-((3-methylisoxazol-5-yl)methoxy)-1H-indol-2-yl)methyl)oxetane-2-carboxamide

To a solution of (R)-oxetane-2-carboxylic acid (559 mg, 5.48 mmol, 1.20 eq) in DMA (10 mL) was added CDI (889 mg, 5.48 mmol, 1.20 eq) in portions at 20° C., and the mixture was stirred at 20° C. for 1 h, whereupon (5-chloro-6-((3-methylisoxazol-5-yl)methoxy)-1H-indol-2-yl)methanamine hydrochloride (1.50 g, 4.57 mmol, 1.00 eq, HCl salt) was added in portions. The resulting mixture was stirred at 20° C. for 1 h, then was quenched by addition of water (10 mL) at 20° C. and extracted with EtOAc (3×15 mL). The combined organic layers were dried over Na₂SO₄, filtered, and concentrated under reduced pressure. The residue was then triturated with EtOH (5 mL) at 20° C. for 30 min and filtered to give (R)—N-((5-chloro-6-((3-methylisoxazol-5-yl)methoxy)-1H-indol-2-yl)methyl)oxetane-2-carboxamide (Compound 317). LC-MS (ESI) m/z calcd for C₁₈H₁₈ClN₃O₄: 375.1; found 376.2 [M+H]⁺. ¹H NMR (400 MHz, CDCl₃) δ 9.05 (s, 1H), 7.56 (s, 1H), 7.47 (t, J=5.2 Hz, 1H), 6.94 (s, 1H), 6.27 (s, 1H), 6.24 (s, 1H), 5.19 (s, 2H), 5.12-5.08 (m, 1H), 4.77-4.75 (m, 1H), 4.59-4.57 (m, 1H), 4.55-4.53 (m, 2H), 3.11-3.04 (m, 1H), 2.68-2.63 (m, 1H), 2.31 (s, 3H).

The following compounds in Table T-31 were synthesized using procedures similar to Compound 317 using the appropriate starting materials.

TABLE T-31

| | | | Exact | LCMS, |
|---|---|---|---|---|
| Compound | | | mass | found |
| No. | Structure | IUPAC name | (g/mol) | [M + H]⁺ |
| 581 | 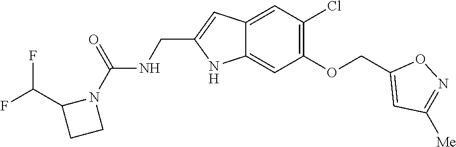 [SMILES: Cc1cc(COc2cc3[nH]c(CNC(=O)N4CCC4C(F)F)cc3cc2Cl)on1] | N-((5-chloro-6-((3- methylisoxazol-5- yl)methoxy)-1H-indol-2- yl)methyl)-2- (difluoromethyl)azetidine- 1-carboxamide | 424.11 | 425.1 |
| 582 | 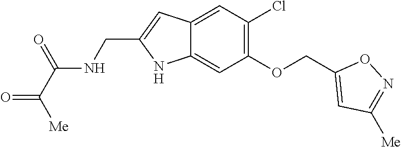 [SMILES: CC(=O)C(=O)NCc1cc2cc(Cl)c(OCc3cc(C)no3)cc2[nH]1] | N-((5-chloro-6-((3- methylisoxazol-5- yl)methoxy)-1H-indol-2- yl)methyl)-2- oxopropanamide | 361.08 | 362.1 |

### Example 53: (Compound 583)

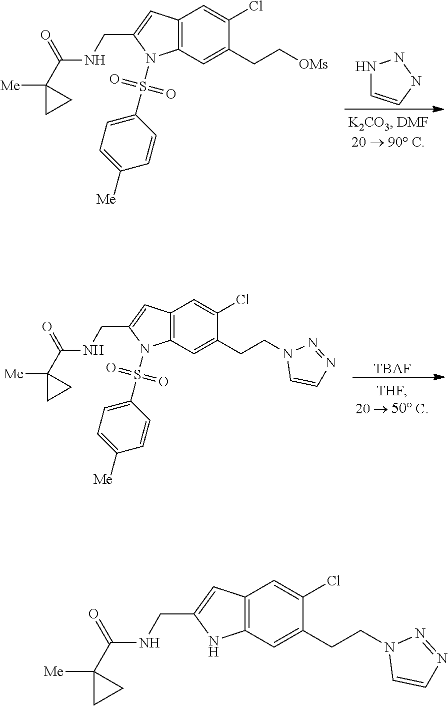

### Step 1: N-((6-(2-(1H-1,2,3-triazol-1-yl)ethyl)-5-chloro-1-tosyl-1H-indol-2-yl)methyl)-1-methylcyclopropane-1-carboxamide3

To a mixture of 2-(5-chloro-2-((1-methylcyclopropane-1-carboxamido)methyl)-1-tosyl-1H-indol-6-yl)ethyl methanesulfonate (300 mg, 556 μmol, 1.00 eq) and 1H-triazole (384 mg, 5.57 mmol, 10.0 eq) in DMF (3 mL) was added K₂CO₃ (307 mg, 2.23 mmol, 4.00 eq) in one portion at 20° C. under N₂. The reaction mixture was heated to 90° C. and stirred at 90° C. for 4 h. After cooling to room temperature, the reaction mixture was quenched by addition of H₂O (30 mL) at 20° C., and extracted with ethyl acetate (3×10 mL). The combined organic layers were washed brine (20 mL), dried over Na₂SO₄, filtered, and concentrated under reduced pressure to give a residue. The residue was purified by column chromatography to give N-((6-(2-(1H-1,2,3-triazol-1-yl)ethyl)-5-chloro-1-tosyl-1H-indol-2-yl)methyl)-1-methylcyclopropane-1-carboxamide. LC-MS (ESI) m/z calcd for C₂₅H₂₆ClN₅O₃S: 511.14; found 512.3 [M+H]⁺.

### Step 2: N-((6-(2-(1H-1,2,3-triazol-1-yl)ethyl)-5-chloro-1H-indol-2-yl)methyl)-1-methylcyclopropane-1-carboxamide

To a solution of N-((6-(2-(1H-1,2,3-triazol-1-yl)ethyl)-5-chloro-1-tosyl-1H-indol-2-yl)methyl)-1-methylcyclopropane-1-carboxamide (125 mg, 244 μmol, 1.00 eq) in THF (5 mL) was added TBAF (1 M in THF, 4.88 mL, 20.0 eq) in one portion at 20° C. under N₂. The reaction mixture was heated to 50° C. and stirred at 50° C. for 16 h. After cooling to room temperature, the reaction mixture was quenched by addition of H₂O (20 mL) and then extracted with ethyl acetate (3×10 mL). The combined organic layers were washed brine (20 mL), dried over Na₂SO₄, filtered, and concentrated under reduced pressure to give a residue. The residue was purified by prep-HPLC to give N-((6-(2-(1H-1,2,3-triazol-1-yl)ethyl)-5-chloro-1H-indol-2-yl)methyl)-1-methylcyclopropane-1-carboxamide (Compound 583). LC-MS (ESI) m/z calcd for C₁₈H₂₀ClN₅O: 357.14; found 358.3 [M+H]⁺. ¹H NMR (400 MHz, DMSO-d₆) δ 10.91 (s, 1H), 8.02 (s, 2H), 7.66 (s, 1H), 7.51 (s, 1H), 7.10 (s, 1H), 6.15 (s, 1H), 4.63 (t, J=7.2 Hz, 2H), 4.36 (d, J=5.6 Hz, 2H), 3.30-3.28 (m, 2H), 1.28 (s, 3H), 1.00-0.97 (m, 2H), 0.54-0.52 (m, 2H).

The following compounds in Table T-32 were synthesized using procedures similar to Compound 583 using the appropriate starting materials.

TABLE T-32

| Com- | | | Exact | LCMS, |
|---|---|---|---|---|
| pound | | | mass | found |
| No. | Structure | IUPAC name | (g/mol) | [M + H]⁺ |
| 584 | 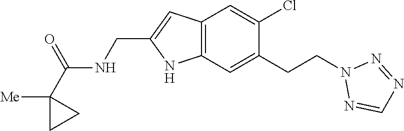 [SMILES: CC1(C(=O)NCc2cc3cc(Cl)c(CCn4ncnn4)cc3[nH]2)CC1] | N-((6-(2-(2H-tetrazol-2- yl)ethyl)-5-chloro-1H- indol-2-yl)methyl)-1- methylcyclopropane-1- carboxamide | 358.13 | 359.2 |
| 585 | 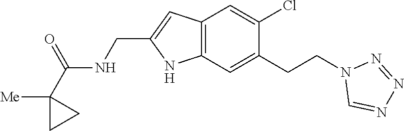 [SMILES: CC1(C(=O)NCc2cc3cc(Cl)c(CCn4cnnn4)cc3[nH]2)CC1] | N-((6-(2-(1H-tetrazol-1- yl)ethyl)-5-chloro-1H- indol-2-yl)methyl)-1- methylcyclopropane-1- carboxamide | 358.13 | 359.2 |
| 586 | 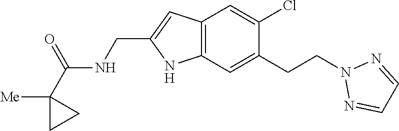 [SMILES: CC1(C(=O)NCc2cc3cc(Cl)c(CCn4nccn4)cc3[nH]2)CC1] | N-((6-(2-(2H-1,2,3- triazol-2-yl)ethyl)-5- chloro-1H-indol-2- yl)methyl)-1- methylcyclopropane-1- carboxamide | 357.14 | 358.3 |
| 587 | 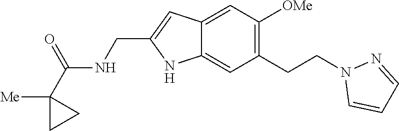 [SMILES: COc1cc2cc(CNC(=O)C3(C)CC3)[nH]c2cc1CCn1cccn1] | N-((6-(2-(1H-pyrazol-1- yl)ethyl)-5-methoxy- 1H-indol-2-yl)methyl)- 1-methylcyclopropane- 1-carboxamide | 352.19 | 353.4 |
| 588 | 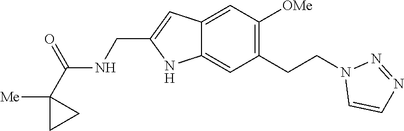 [SMILES: COc1cc2cc(CNC(=O)C3(C)CC3)[nH]c2cc1CCn1ccnn1] | N-((6-(2-(1H-1,2,3- triazol-1-yl)ethyl)-5- methoxy-1H-indol-2- yl)methyl)-1- methylcyclopropane-1- carboxamide | 353.19 | 354.4 |
| 589 | 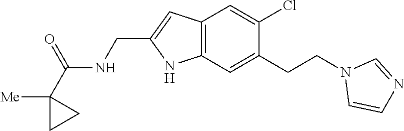 [SMILES: CC1(C(=O)NCc2cc3cc(Cl)c(CCn4ccnc4)cc3[nH]2)CC1] | N-((6-(2-(1H-imidazol- 1-yl)ethyl)-5-chloro-1H- indol-2-yl)methyl)-1- methylcyclopropane-1- carboxamide | 356.14 | 357.2 |
| 590 | 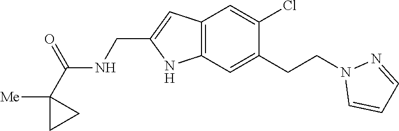 [SMILES: CC1(C(=O)NCc2cc3cc(Cl)c(CCn4cccn4)cc3[nH]2)CC1] | N-((6-(2-(1H-pyrazol-1- yl)ethyl)-5-chloro-1H- indol-2-yl)methyl)-1- methylcyclopropane-1- carboxamide | 356.14 | 357.2 |
| 591 | 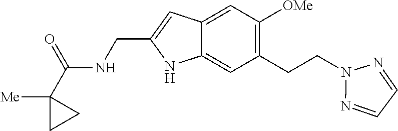 [SMILES: COc1cc2cc(CNC(=O)C3(C)CC3)[nH]c2cc1CCn1nccn1] | N-((6-(2-(2H-1,2,3- triazol-2-yl)ethyl)-5- methoxy-1H-indol-2- yl)methyl)-1- methylcyclopropane-1- carboxamide | 353.19 | 354.4 |

### Example 54: (Compound 592)

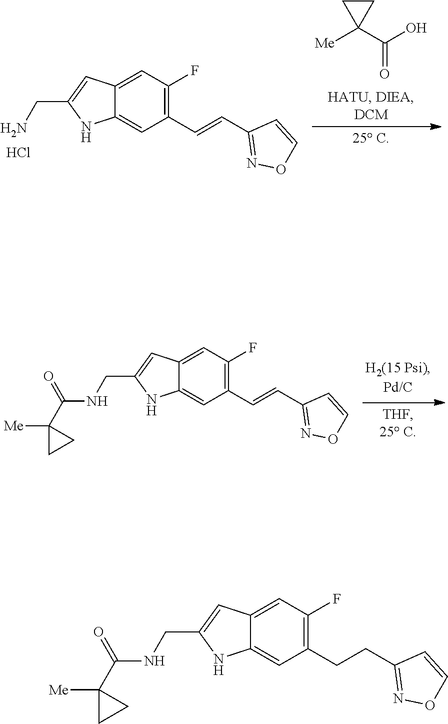 [SMILES: Cl ; NCc1cc2cc(F)c(/C=C/c3ccon3)cc2[nH]1 ; CC1(C(=O)O)CC1 ; CC1(C(=O)NCc2cc3cc(F)c(/C=C/c4ccon4)cc3[nH]2)CC1 ; CC1(C(=O)NCc2cc3cc(F)c(CCc4ccon4)cc3[nH]2)CC1]

### Step 1: (E)-N-((5-fluoro-6-(2-(isoxazol-3-yl)vinyl)-1H-indol-2-yl)methyl)-1-methylcyclopropane-1-carboxamide

To a solution of (E)-(5-fluoro-6-(2-(isoxazol-3-yl)vinyl)-1H-indol-2-yl)methanamine hydrochloride (200 mg, 680 μmol, 1.00 eq, HCl salt) in DCM (2 mL) were added 1-methylcyclopropanecarboxylic acid (81.8 mg, 817 μmol, 1.20 eq), DIEA (528 mg, 4.09 mmol, 711 L, 6.00 eq), and HATU (336 mg, 885 μmol, 1.30 eq) in one portion. The reaction mixture was stirred at 25° C. for 1 h. The reaction mixture was quenched by addition of H₂O (5 mL) and extracted with DCM (3×10 mL). The combined organic layers were washed with brine (5 mL), dried over Na₂SO₄, filtered, and concentrated under reduced pressure to give a residue. The residue was purified by column chromatography to give (E)-N-((5-fluoro-6-(2-(isoxazol-3-yl)vinyl)-1H-indol-2-yl)methyl)-1-methylcyclopropane-1-carboxamide. LC-MS (ESI) m/z calcd for C₁₉H₁₈FN₃O₂: 339.14; found: 340.2 [M+H]⁺.

### Step 2: N-((5-fluoro-6-(2-(isoxazol-3-yl)ethyl)-1H-indol-2-yl)methyl)-1-methylcyclopropane-1-carboxamide

To a slurry of Pd/C (200 mg, 294 μmol, 10.0% purity, 1.00 eq) in THF (1 mL) was added (E)-N-((5-fluoro-6-(2-(isoxazol-3-yl)vinyl)-1H-indol-2-yl)methyl)-1-methylcyclopropane-1-carboxamide (100 mg, 294 μmol, 1.00 eq) in one portion, and the reaction mixture was degassed and purged with H₂ three times. The reaction mixture was stirred at 25° C. for 5 mins under H₂ (15 psi) atmosphere. The reaction mixture was filtered through a Celite pad and washed with MeOH (3×10 mL), and the organic layer was concentrated under reduced pressure to give a residue. The residue was purified by prep-HPLC to give N-((5-fluoro-6-(2-(isoxazol-3-yl)ethyl)-1H-indol-2-yl)methyl)-1-methylcyclopropane-1-carboxamide (Compound 592). LC-MS (ESI) m/z calcd for C₁₉H₁₈FN₃O₂: 341.15; found: 342.2 [M+H]⁺. ¹H NMR (400 MHz, DMSO-d₆) δ 10.77 (s, 1H), 8.77 (d, J=1.2 Hz, 1H), 8.01 (t, J=6.0 Hz, 1H), 7.18 (d, J=5.6 Hz, 1H), 7.14 (s, 1H), 6.49 (s, 1H), 6.14 (s, 1H), 4.36 (d, J=5.6 Hz, 2H), 3.03-2.97 (m, 2H), 2.97-2.93 (m, 2H), 1.28 (s, 3H), 1.00-0.98 (m, 2H), 0.54-0.51 (m, 2H).

### Example 55: (Compound 593)

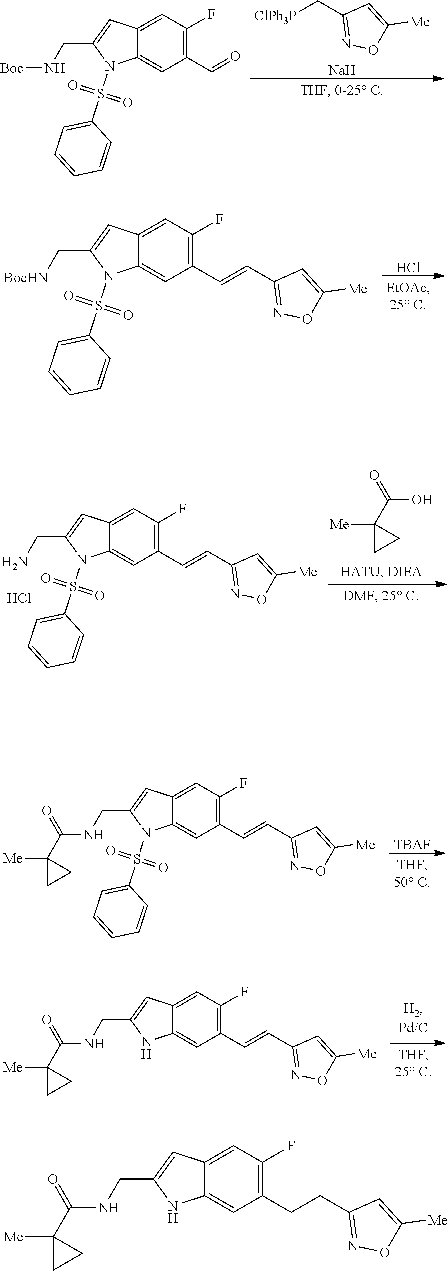

### Step 1: tert-butyl (E)-((5-fluoro-6-(2-(5-methylisoxazol-3-yl)vinyl)-1-(phenylsulfonyl)-1H-indol-2-yl)methyl)carbamate

To a solution of sodium hydride (NaH, 90.1 mg, 2.25 mmol, 1.30 eq) in THF (5 mL) at 0° C. was added 3-((chlorotriphenyl-λ⁵-phosphanyl)methyl)-5-methylisoxazole (683 mg, 1.73 mmol, 1.00 eq) in portions. After 1 h at 0° C., a solution of tert-butyl ((5-fluoro-6-formyl-1-(phenylsulfonyl)-1H-indol-2-yl)methyl)carbamate (750 mg, 1.73 mmol, 1.00 eq) in THF (5 mL) was added dropwise. The reaction was allowed to warm to 25° C. After 2 h the reaction was quenched with sat. aq. NH₄Cl (15 mL) at 0° C. The solution was extracted with ethyl acetate (3×15 mL). The combined organic layers were washed with brine (15 mL), dried over anhydrous Na₂SO₄, filtered, and concentrated under reduced pressure. Purification by column chromatography gave tert-butyl (E)-((5-fluoro-6-(2-(5-methylisoxazol-3-yl)vinyl)-1-(phenylsulfonyl)-1H-indol-2-yl)methyl)carbamate. LC-MS (ESI) m/z calcd for C₂₆H₂₆FN₃O₅S: 511.2; found: 456.2 [M−tBu+2H]⁺.

### Step 2: (E)-(5-fluoro-6-(2-(5-methylisoxazol-3-yl)vinyl)-1-(phenylsulfonyl)-1H-indol-2-yl)methanamine hydrochloride

To a solution of tert-butyl (E)-((5-fluoro-6-(2-(5-methylisoxazol-3-yl)vinyl)-1-(phenylsulfonyl)-1H-indol-2-yl)methyl)carbamate (780 mg, 1.52 mmol, 1.00 eq) in ethyl acetate (0.2 mL) was added HCl in ethyl acetate (4 M, 39.0 mL, 102 eq). After 2 h the reaction mixture was concentrated under reduced pressure to give (E)-(5-fluoro-6-(2-(5-methylisoxazol-3-yl)vinyl)-1-(phenylsulfonyl)-1H-indol-2-yl)methanamine hydrochloride, which was used directly without further purification. LC-MS (ESI) m/z calcd for C₂₁H₁₈FN₃O₃S: 411.1; found: 395.2 [M−NH₂]⁺.

### Step 3: (E)-N-((5-fluoro-6-(2-(5-methylisoxazol-3-yl)vinyl)-1-(phenylsulfonyl)-1H-indol-2-yl)methyl)-1-methylcyclopropane-1-carboxamide

To a mixture of (E)-(5-fluoro-6-(2-(5-methylisoxazol-3-yl)vinyl)-1-(phenylsulfonyl)-1H-indol-2-yl)methanamine hydrochloride (630 mg, 1.41 mmol, 1.00 eq) in DMF (5 mL) was added 1-methylcyclopropane-1-carboxylic acid (154 mg, 1.55 mmol, 1.10 eq), DIEA (545 mg, 4.22 mmol, 3.00 eq), and HATU (695 mg, 1.83 mmol, 1.30 eq). After 2 h the reaction was quenched by addition of H₂O (5 mL). The solution was extracted with ethyl acetate (3×10 mL). The combined organic layers were washed with brine (10 mL), dried over anhydrous Na₂SO₄, filtered, and concentrated under reduced pressure. Purification by column chromatography gave (E)-N-((5-fluoro-6-(2-(5-methylisoxazol-3-yl)vinyl)-1-(phenylsulfonyl)-1H-indol-2-yl)methyl)-1-methylcyclopropane-1-carboxamide. LC-MS (ESI) m/z calcd for C₂₆H₂₄FN₃O₄S: 493.1; found: 494.3 [M+H]⁺.

### Step 4: (E)-N-((5-fluoro-6-(2-(5-methylisoxazol-3-yl)vinyl)-1H-indol-2-yl)methyl)-1-methylcyclopropane-1-carboxamide

To a solution of (E)-N-((5-fluoro-6-(2-(5-methylisoxazol-3-yl)vinyl)-1-(phenylsulfonyl)-1H-indol-2-yl)methyl)-1-methylcyclopropane-1-carboxamide (580 mg, 1.18 mmol, 1.00 eq) in THF (6 mL) was added TBAF (5.88 mL, 5.00 eq) dropwise. The reaction mixture was heated to 50° C. After 2 h the reaction was quenched by addition of H₂O (10 mL) at 25° C. The solution was extracted with ethyl acetate (3×10 mL). The combined organic layers were washed with brine (3×10 mL), dried over anhydrous Na₂SO₄, filtered, and concentrated under reduced pressure to give (E)-N-((5-fluoro-6-(2-(5-methylisoxazol-3-yl)vinyl)-1H-indol-2-yl)methyl)-1-methylcyclopropane-1-carboxamide, which was used directly without further purification. LC-MS (ESI) m/z calcd for C₂₀H₂₀FN₃O₂: 353.2; found: 354.3 [M+H]⁺.

### Step 5: N-((5-fluoro-6-(2-(5-methylisoxazol-3-yl)ethyl)-1H-indol-2-yl)methyl)-1-methylcyclopropane-1-carboxamide

To a solution of (E)-N-((5-fluoro-6-(2-(5-methylisoxazol-3-yl)vinyl)-1H-indol-2-yl)methyl)-1-methylcyclopropane-1-carboxamide (150 mg, 424 μmol, 1.00 eq) in THF (1.5 mL) was added Pd/C (150 mg, 15.0 mmol, 35.3 eq). The suspension was degassed and purged with H₂ three times. After 40 min at 15 Psi H₂, the reaction mixture was filtered through Celite and concentrated under reduced pressure. Purification by prep-HPLC gave N-((5-fluoro-6-(2-(5-methylisoxazol-3-yl)ethyl)-1H-indol-2-yl)methyl)-1-methylcyclopropane-1-carboxamide (Compound 593). LC-MS (ESI) m/z calcd for C₂₀H₂₂FN₃O₂: 355.2; found: 356.3 [M+H]⁺. ¹H NMR (400 MHz, DMSO-d₆) δ 10.77 (s, 1H), 8.01 (t, J=6.0 Hz, 1H), 7.19-7.14 (m, 2H), 6.14 (s, 2H), 4.36 (d, J=5.6 Hz, 2H), 3.00-2.95 (m, 2H), 2.88-2.84 (m, 2H), 2.34 (s, 3H), 1.28 (s, 3H), 1.00-0.98 (m, 2H), 0.54-0.51 (m, 2H).

The following compounds in Table T-33 were synthesized using procedures similar to Compound 593 using the appropriate starting materials.

TABLE T-33

| Com- | | | Exact | LCMS, |
|---|---|---|---|---|
| pound | | | mass | found |
| No. | Structure | IUPAC name | (g/mol) | [M + H]⁺ |
| 594 | 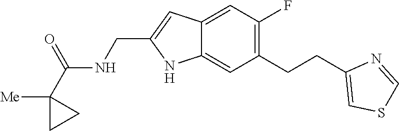 [SMILES: CC1(C(=O)NCc2cc3cc(F)c(CCc4cscn4)cc3[nH]2)CC1] | N-((5-fluoro-6-(2- (thiazol-4-yl)ethyl)-1H- indol-2-yl)methyl)-1- methylcyclopropane-1- carboxamide | 357.13 | 358.3 |

### Example 56 (Compound 595)

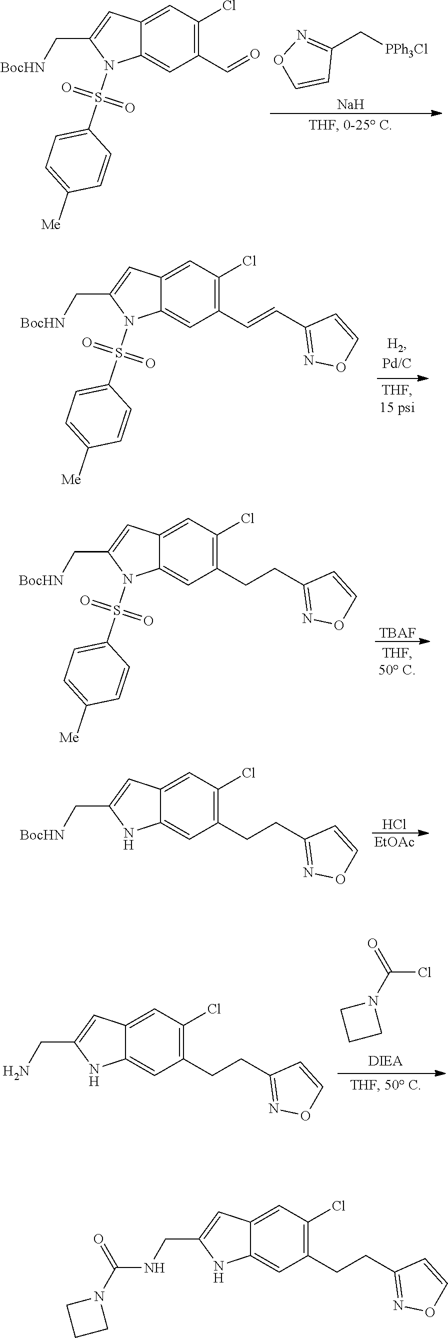

### Step 1: tert-butyl (E)-((5-chloro-6-(2-(isoxazol-3-yl)vinyl)-1-tosyl-1H-indol-2-yl)methyl)carbamate

To a solution of 3-((chlorotriphenyl-λ⁵-phosphanyl)methyl)isoxazole (328 mg, 864 μmol, 1.00 eq) in THF (8 mL) was added NaH (60% w/w, 45.0 mg, 1.12 mmol, 1.30 eq) at 0° C., and the reaction was stirred at 0° C. for 30 min. A solution of tert-butyl ((5-chloro-6-formyl-1-tosyl-1H-indol-2-yl)methyl)carbamate (400 mg, 864 μmol, 1.00 eq) in THF (4 mL) was added and the reaction was warmed to 25° C. and stirred for 2 h. The reaction mixture was poured into water (10 mL) at 0° C. The mixture was extracted with EtOAc (3×10 mL). The combined organic layers were washed with brine (3×10 mL), dried over anhydrous Na₂SO₄, filtered, and concentrated under reduced pressure. The cruel material was purified by column chromatography to give tert-butyl (E)-((5-chloro-6-(2-(isoxazol-3-yl)vinyl)-1-tosyl-1H-indol-2-yl)methyl)carbamate. LC-MS (ESI) m/z calcd for C₂₆H₂₆ClN₃O₅S: 527.13; found: 528.2 [M+H]⁺.

### Step 2: tert-butyl ((5-chloro-6-(2-(isoxazol-3-yl)ethyl)-1-tosyl-1H-indol-2-yl)methyl)carbamate

To a solution of tert-butyl (E)-((5-chloro-6-(2-(isoxazol-3-yl)vinyl)-1-tosyl-1H-indol-2-yl)methyl)carbamate (300 mg, 568 μmol, 1.00 eq) in THF (0.5 mL) was added Pd/C (10% w/w, 300 mg, 277 μmol, 0.5 eq). The mixture was degassed and purged with H₂ three times. The reaction mixture was stirred under H₂ (15 psi) at 25° C. for 5 min. The reaction mixture was filtered through Celite and concentrated under reduced pressure. The crude material was purified by column chromatography to give tert-butyl ((5-chloro-6-(2-(isoxazol-3-yl)ethyl)-1-tosyl-1H-indol-2-yl)methyl)carbamate. LC-MS (ESI) m/z calcd for C₂₆H₂₈ClN₃O₅S: 529.14; found: 552.2 [M+Na]⁺.

### Step 3: tert-butyl ((5-chloro-6-(2-(isoxazol-3-yl)ethyl)-1H-indol-2-yl)methyl)carbamate

To a solution of tert-butyl ((5-chloro-6-(2-(isoxazol-3-yl)ethyl)-1-tosyl-1H-indol-2-yl)methyl)carbamate (150 mg, 283 μmol, 1.00 eq) in THF (0.5 mL) was added TBAF (1 M in THF, 2 mL, 2.00 mmol, 7.07 eq), and the reaction was heated to 50° C. for 1 h. The reaction mixture was cooled to 25° C. and was poured into water (10 mL) at 0° C. The resulting mixture was extracted with EtOAc (3×10 mL). The combined organic layers were washed with sat. aq. NH₄Cl (3×10 mL), dried over anhydrous Na₂SO₄, filtered, and concentrated under reduced pressure to give tert-butyl ((5-chloro-6-(2-(isoxazol-3-yl)ethyl)-1H-indol-2-yl)methyl)carbamate. LC-MS (ESI) m/z calcd for C₁₉H₂₂ClN₃O₃: 375.13; found: 376.2 [M+H]⁺.

### Step 4: (5-chloro-6-(2-(isoxazol-3-yl)ethyl)-1H-indol-2-yl)methanamine

To a solution of a solution of tert-butyl ((5-chloro-6-(2-(isoxazol-3-yl)ethyl)-1H-indol-2-yl)methyl)carbamate (100 mg, 266 μmol, 1.00 eq) in EtOAc (0.5 mL) was added HCl (4 M in EtOAc, 5 mL), and the reaction was stirred at 25° C. for 1 h. The reaction mixture was concentrated under reduced pressure to give (5-chloro-6-(2-(isoxazol-3-yl)ethyl)-1H-indol-2-yl)methanamine, which was used directly without further purification. LC-MS (ESI) m/z calcd for C₁₄H₁₄ClN₃O₃: 275.08; found: 276.1 [M+H]⁺.

### Step 5: N-((5-chloro-6-(2-(isoxazol-3-yl)ethyl)-1H-indol-2-yl)methyl)azetidine-1-carboxamide

To a solution of (5-chloro-6-(2-(isoxazol-3-yl)ethyl)-1H-indol-2-yl)methanamine (91.0 mg, 291 μmol, 1.00 eq) in THF (2.0 mL) were added DIEA (150 mg, 1.17 mmol, 4.00 eq) and azetidine-1-carbonyl chloride (104 mg, 874 μmol, 3.00 eq) at 0° C. The reaction mixture was then heated to 50° C. for 2 h. The reaction mixture was cooled to 25° C. and poured into water (10 mL). The resulting mixture was extracted with EtOAc (3×10 mL). The combined organic layers were washed with brine (3×10 mL), dried over anhydrous Na₂SO₄, filtered, and concentrated under reduced pressure. The crude materials was purified by prep-HPLC to give N-((5-chloro-6-(2-(isoxazol-3-yl)ethyl)-1H-indol-2-yl)methyl)azetidine-1-carboxamide (Compound 595). LC-MS (ESI) m/z calcd for C₁₈H₁₉ClN₄O₂: 358.12; found: 359.3 [M+H]⁺. ¹H NMR (400 MHz, DMSO-d₆) δ 10.88 (s, 1H), 8.78 (d, J=1.2 Hz, 1H), 7.48 (s, 1H), 7.25 (s, 1H), 6.75 (t, J=6.0 Hz, 1H), 6.51 (s, 1H), 6.16 (s, 1H), 4.28 (d, J=6.0 Hz, 2H), 3.84-3.80 (m, 4H), 3.10-3.07 (m, 2H), 2.98-2.94 (m, 2H), 2.17-2.09 (m, 2H).

The following compounds in Table T-34 were synthesized using procedures similar to Compound 595 using the appropriate starting materials.

TABLE T-34

| Com- | | | Exact | LCMS, |
|---|---|---|---|---|
| pound | | | mass | found |
| No. | Structure | IUPAC name | (g/mol) | [M + H]⁺ |
| 596 | 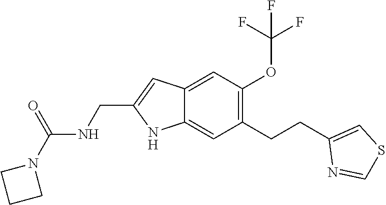 [SMILES: O=C(NCc1cc2cc(OC(F)(F)F)c(CCc3cscn3)cc2[nH]1)N1CCC1] | N-((6-(2-(thiazol-4- yl)ethyl)-5- (trifluoromethoxy)-1H- indol-2- yl)methyl)azetidine-1- carboxamide | 424.12 | 425.1 |
| 597 | 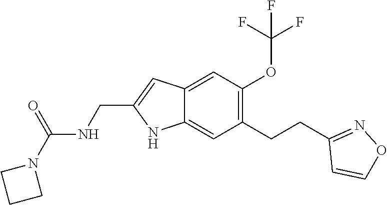 [SMILES: O=C(NCc1cc2cc(OC(F)(F)F)c(CCc3ccon3)cc2[nH]1)N1CCC1] | N-((6-(2-(isoxazol-3- yl)ethyl)-5- (trifluoromethoxy)-1H- indol-2- yl)methyl)azetidine-1- carboxamide | 408.14 | 409.1 |
| 598 | 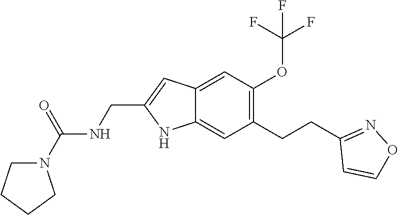 [SMILES: O=C(NCc1cc2cc(OC(F)(F)F)c(CCc3ccon3)cc2[nH]1)N1CCCC1] | N-((6-(2-(isoxazol-3- yl)ethyl)-5- (trifluoromethoxy)-1H- indol-2- yl)methyl)pyrrolidine-1- carboxamide | 422.16 | 423.1 |
| 599 | 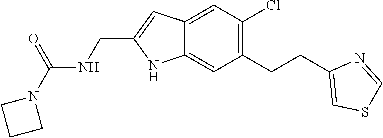 [SMILES: O=C(NCc1cc2cc(Cl)c(CCc3cscn3)cc2[nH]1)N1CCC1] | N-((5-chloro-6-(2- (thiazol-4-yl)ethyl)-1H- indol-2- yl)methyl)azetidine-1- carboxamide | 374.10 | 375.3 |

### Example 57 (Compound 600)

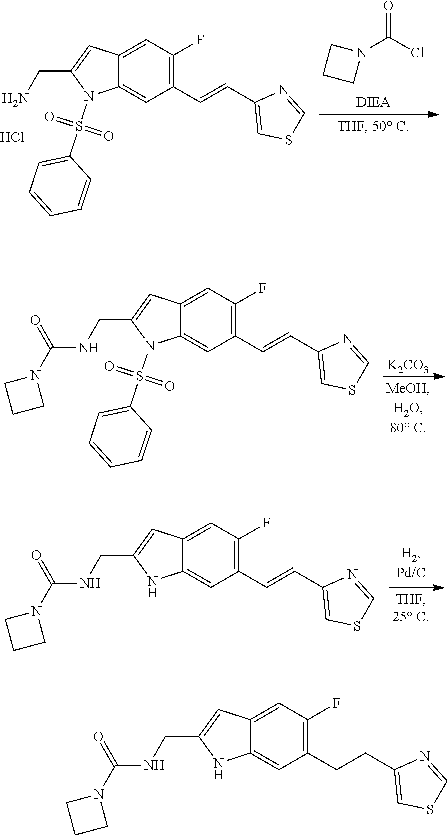

### Step 1: (E)-N-((5-fluoro-1-(phenylsulfonyl)-6-(2-(thiazol-4-yl)vinyl)-1H-indol-2-yl)methyl)azetidine-1-carboxamide

To a solution of (E)-(5-fluoro-1-(phenylsulfonyl)-6-(2-(thiazol-4-yl)vinyl)-1H-indol-2-yl)methanamine hydrochloride (400 mg, 967 μmol, 1.00 eq) in THF (5 mL) was added DIEA (375 mg, 2.90 mmol, 3.00 eq) and azetidine-1-carbonyl chloride (346 mg, 2.90 mmol, 3.00 eq). The reaction mixture was heated to 50° C. After 1 h, the reaction was quenched by addition of H₂O (10 mL). The solution was extracted with EtOAc (3×10 mL). The combined organic layers were washed with brine (10 mL), dried over anhydrous Na₂SO₄, filtered, and concentrated under reduced pressure. Purification by column chromatography gave (E)-N-((5-fluoro-1-(phenylsulfonyl)-6-(2-(thiazol-4-yl)vinyl)-1H-indol-2-yl)methyl)azetidine-1-carboxamide. LC-MS (ESI) m/z calcd for C₂₄H₂₁FN₄O₃S₂: 496.1; found 497.2 [M+H]⁺.

### Step 2: (E)-N-((5-fluoro-6-(2-(thiazol-4-yl)vinyl)-1H-indol-2-yl)methyl)azetidine-1-carboxamide

To a solution of (E)-N-((5-fluoro-1-(phenylsulfonyl)-6-(2-(thiazol-4-yl)vinyl)-1H-indol-2-yl)methyl)azetidine-1-carboxamide (350 mg, 704 μmol, 1.00 eq) in MeOH (5 mL) and H₂O (0.5 mL) was added K₂CO₃ (292 mg, 2.11 mmol, 3.00 eq) in one portion. The reaction mixture was heated to 80° C. After 16 h, the reaction was quenched by addition of H₂O (5 mL). The solution was extracted with EtOAc (3×5 mL). The combined organic layers were washed with brine (5 mL), dried over anhydrous Na₂SO₄, filtered, and concentrated under reduced pressure to give (E)-N-((5-fluoro-6-(2-(thiazol-4-yl)vinyl)-1H-indol-2-yl)methyl)azetidine-1-carboxamide. LC-MS (ESI) m/z calcd for C18H₁₇FN₄OS: 356.1; found 357.2 [M+H]⁺.

### Step 3: N-((5-fluoro-6-(2-(thiazol-4-yl)ethyl)-1H-indol-2-yl)methyl)azetidine-1-carboxamide

To a solution of (E)-N-((5-fluoro-6-(2-(thiazol-4-yl)vinyl)-1H-indol-2-yl)methyl)azetidine-1-carboxamide (160 mg, 448 μmol, 1.00 eq) in THF (2 mL) was added Pd/C (160 mg, 16.0 mmol, 35.6 eq) under N₂ atmosphere. The suspension was degassed and purged with H₂ three times. After 2 h at 15 Psi H₂, the reaction mixture was filtered through Celite and concentrated under reduced pressure. Purification by prep-HPLC gave N-((5-fluoro-6-(2-(thiazol-4-yl)ethyl)-1H-indol-2-yl)methyl)azetidine-1-carboxamide (Compound 600). LC-MS (ESI) m/z calcd for C18H₁₉FN₄OS: 358.1; found 359.3 [M+H]⁺. ¹H NMR (400 MHz, DMSO-d₆) δ 10.72 (s, 1H), 9.03 (d, J=2.0 Hz, 1H), 7.32 (d, J=2.0 Hz, 1H), 7.15-7.12 (m, 2H), 6.72 (t, J=6.0 Hz, 1H), 6.14 (d, J=1.2 Hz, 1H), 4.27 (d, J=6.0 Hz, 2H), 3.82 (t, J=7.2 Hz, 4H), 3.04 (s, 4H), 2.16-2.09 (m, 2H).

The following compounds in Table T-35 were synthesized using procedures similar to Compound 600 using the appropriate starting materials.

TABLE T-35

| Com- | | | Exact | LCMS, |
|---|---|---|---|---|
| pound | | | mass | found |
| No. | Structure | IUPAC name | (g/mol) | [M + H]⁺ |
| 601 | 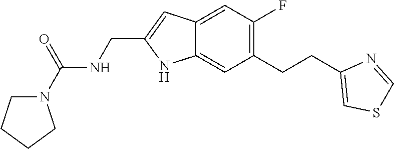 [SMILES: O=C(NCc1cc2cc(F)c(CCc3cscn3)cc2[nH]1)N1CCCC1] | N-((5-fluoro-6-(2- (thiazol-4-yl)ethyl)-1H- indol-2- yl)methyl)pyrrolidine-1- carboxamide | 372.14 | 373.3 |

### Example 58 (Compound 602)

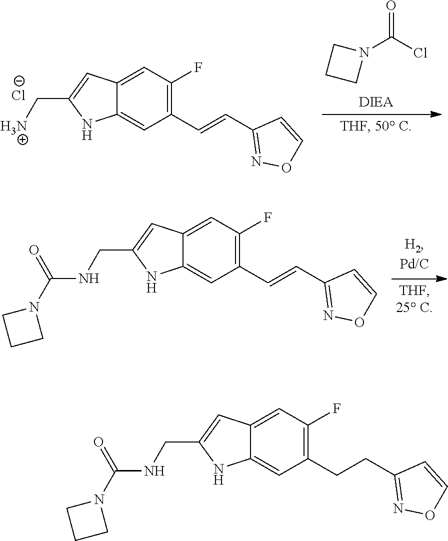 [SMILES: [Cl-] ; [NH3+]Cc1cc2cc(F)c(/C=C/c3ccon3)cc2[nH]1 ; O=C(Cl)N1CCC1 ; O=C(NCc1cc2cc(F)c(/C=C/c3ccon3)cc2[nH]1)N1CCC1 ; O=C(NCc1cc2cc(F)c(CCc3ccon3)cc2[nH]1)N1CCC1]

### Step 1: (E)-N-((5-fluoro-6-(2-(isoxazol-3-yl)vinyl)-1H-indol-2-yl)methyl)azetidine-1-carboxamide

To a solution of (E)-(5-fluoro-6-(2-(isoxazol-3-yl)vinyl)-1H-indol-2-yl)methanamine hydrochloride (260 mg, 1.01 mmol, 1.00 eq) in THF (3 mL) was added DIEA (391 mg, 3.03 mmol, 3.00 eq) and azetidine-1-carbonyl chloride (362 mg, 3.03 mmol, 3.00 eq). The reaction mixture was heated to 50° C. After 2 h, the reaction was quenched by addition of H₂O (10 mL) at 25° C. The solution was extracted with ethyl acetate (3×10 mL). The combined organic layers were washed with brine (10 mL), dried over anhydrous Na₂SO₄, filtered, and concentrated under reduced pressure to give (E)-N-((5-fluoro-6-(2-(isoxazol-3-yl)vinyl)-1H-indol-2-yl)methyl)azetidine-1-carboxamide which was used directly without further purification. LC-MS (ESI) m/z calcd for C₁₈H₁₇FN₄O₂: 340.1; found 341.2 [M+H]⁺.

### Step 2: N-((5-fluoro-6-(2-(isoxazol-3-yl)ethyl)-1H-indol-2-yl)methyl)azetidine-1-carboxamide

To a solution of (E)-N-((5-fluoro-6-(2-(isoxazol-3-yl)vinyl)-1H-indol-2-yl)methyl)azetidine-1-carboxamide (150 mg, 440 μmol, 1.00 eq) in THF (2 mL) was added Pd/C (150 mg, 440 μmol 1.00 eq) under N₂ atmosphere. The reaction mixture was degassed and purged with H₂ three times. After 30 min at 15 Psi H₂, the reaction mixture was filtered through Celite and concentrated under reduced pressure. Purification by prep-HPLC gave N-((5-fluoro-6-(2-(isoxazol-3-yl)ethyl)-1H-indol-2-yl)methyl)azetidine-1-carboxamide (Compound 602). LC-MS (ESI) m/z calcd for C₁₈H₁₉FN₄O₂: 342.1; found 343.3 [M+H]⁺. ¹H NMR (400 MHz, DMSO-d₆) δ 10.77 (s, 1H), 8.76 (d, J=1.6 Hz, 1H), 7.17-7.13 (m, 2H), 6.73 (t, J=5.6 Hz, 1H), 6.48 (d, J=1.6 Hz, 1H), 6.15 (s, 1H), 4.27 (d, J=6.0 Hz, 2H), 3.82 (t, J=7.6 Hz, 4H), 3.02-2.99 (m, 2H), 2.96-2.94 (m, 2H), 2.16-2.09 (m, 2H).

The following compounds in Table T-36 were synthesized using procedures similar to Compound 602 using the appropriate starting materials.

TABLE T-36

| Com- | | | Exact | LCMS, |
|---|---|---|---|---|
| pound | | | mass | found |
| No. | Structure | IUPAC name | (g/mol) | [M + H]⁺ |
| 603 | 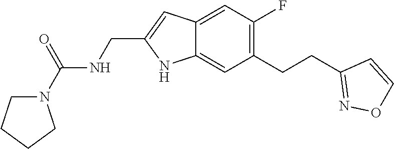 [SMILES: O=C(NCc1cc2cc(F)c(CCc3ccon3)cc2[nH]1)N1CCCC1] | N-((5-fluoro-6-(2- yl)methyl)pyrrolidine-1- 1H-indol-2- (isoxazol-3-yl)ethyl)- carboxamide | 356.16 | 357.2 |

### Example 59 (Compound 604)

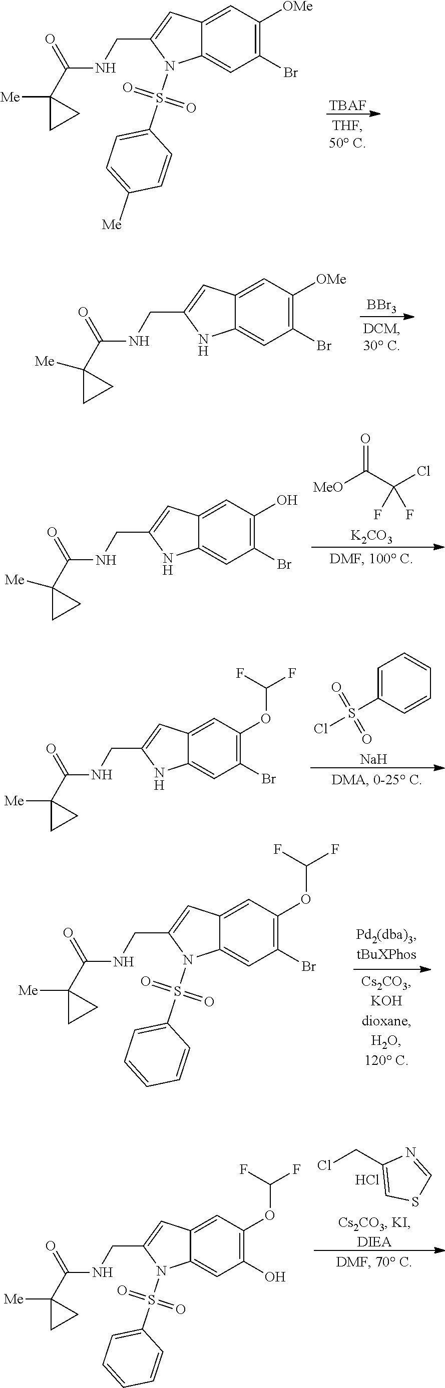

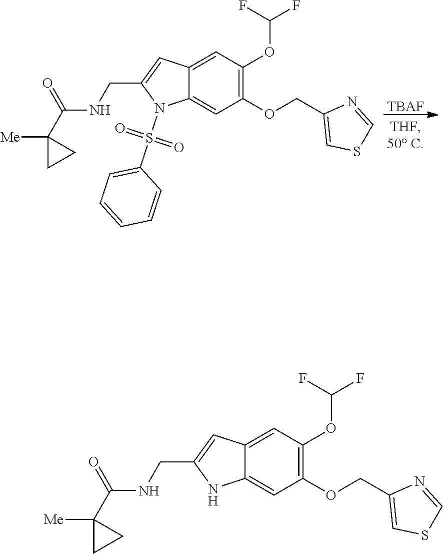

### Step 1: N-((6-bromo-5-methoxy-1H-indol-2-yl)methyl)-1-methylcyclopropane-1-carboxamide

To a solution of N-((6-bromo-5-methoxy-1-tosyl-1H-indol-2-yl)methyl)-1-methylcyclopropane-1-carboxamide (3.2 g, 6.51 mmol, 1.00 eq) in THF (10 mL) was added TBAF (1 M in THF, 26 mL, 26 mmol, 4.00 eq). The mixture was stirred at 50° C. for 12 h. The reaction mixture was cooled to room temperature and poured into water (15 mL). The resulting mixture was then extracted with EtOAc (3×20 mL). The combined organic layers were washed with sat. aq. NH₄Cl (3×20 mL), dried over anhydrous Na₂SO₄, filtered, and concentrated under reduced pressure. Purification by column chromatography gave N-((6-bromo-5-methoxy-1H-indol-2-yl)methyl)-1-methylcyclopropane-1-carboxamide. LC-MS (ESI) m/z calcd for C₁₅H₁₇BrN₂O₂: 336.05; found: 337.0 [M+H]⁺.

### Step 2: N-((6-bromo-5-hydroxy-1H-indol-2-yl)methyl)-1-methylcyclopropane-1-carboxamide

To a solution of N-((6-bromo-5-methoxy-1H-indol-2-yl)methyl)-1-methylcyclopropane-1-carboxamide (1.9 g, 5.63 mmol, 1.00 eq) in DCM (20 mL) was added BBr₃ (3.26 mL, 33.8 mmol, 6.00 eq). The mixture was stirred at 30° C. for 3 h. The reaction mixture was cooled to room temperature and poured slowly into ice water (10 mL). The resulting mixture was stirred for 20 min, and was adjusted to pH=8 with the addition of sat. aq. NaHCO₃. The mixture was extracted with EtOAc (3×15 mL). The combined organic layers were washed with brine (15 mL), dried over anhydrous Na₂SO₄, filtered, and concentrated under reduced pressure. Purification by column chromatography gave N-((6-bromo-5-hydroxy-1H-indol-2-yl)methyl)-1-methylcyclopropane-1-carboxamide. LC-MS (ESI) m/z calcd for C₁₄H₁₅BrN₂O₂: 322.03; found: 323.1 [M+H]⁺.

### Step 3: N-((6-bromo-5-(difluoromethoxy)-1H-indol-2-yl)methyl)-1-methylcyclopropane-1-carboxamide

To a solution of N-((6-bromo-5-hydroxy-1H-indol-2-yl)methyl)-1-methylcyclopropane-1-carboxamide (650 mg, 2.01 mmol, 1.00 eq) in DMF (5 mL) was added K₂CO₃ (280 mg, 2.01 mmol, 1.00 eq). The mixture was stirred at 25° C. for 15 min. Methyl 2-chloro-2,2-difluoroacetate (377 mg, 2.61 mmol, 1.3 eq) was added and the reaction was heated to 100° C. for 1 h. The reaction mixture was cooled to room temperature and quenched with water (20 mL). The resulting mixture was extracted with EtOAc (3×10 mL). The combined organic layers were washed with brine (10 mL), dried over anhydrous Na₂SO₄, filtered, and concentrated under reduced pressure. Purification by column chromatography gave N-((6-bromo-5-(difluoromethoxy)-1H-indol-2-yl)methyl)-1-methylcyclopropane-1-carboxamide. LC-MS (ESI) m/z calcd for C₁₅H₁₅BrF₂N₂O₂: 372.03; found: 373.0 [M+H]⁺.

### Step 4: N-((6-bromo-5-(difluoromethoxy)-1-(phenylsulfonyl)-1H-indol-2-yl)methyl)-1-methylcyclopropane-1-carboxamide

To a solution of N-((6-bromo-5-(difluoromethoxy)-1H-indol-2-yl)methyl)-1-methylcyclopropane-1-carboxamide (650 mg, 0.59 mmol, 1.00 eq) in DMA (2 mL) at 0° C. was added NaH (60% w/w, 47.2 mg, 1.18 mmol, 2.00 eq). The mixture was stirred at 0° C. for 30 min. Benzenesulfonyl chloride (124 mg, 0.71 mmol, 1.2 eq) was added and the reaction was warmed to 25° C. and stirred for 30 min. The reaction mixture was slowly poured into ice water (10 mL) and the resulting mixture was stirred for 3 min. The mixture was extracted with EtOAc (2×15 mL). The combined organic layers were washed with brine (2×15 mL), dried over anhydrous Na₂SO₄, filtered, and concentrated under reduced pressure. Purification by column chromatography gave N-((6-bromo-5-(difluoromethoxy)-1-(phenylsulfonyl)-1H-indol-2-yl)methyl)-1-methylcyclopropane-1-carboxamide. LC-MS (ESI) m/z calcd for C₂₁H₁₉BrF₂N₂O₄S: 512.02; found: 513.0 [M+H]⁺.

### Step 5: N-((5-(difluoromethoxy)-6-hydroxy-1-(phenylsulfonyl)-1H-indol-2-yl)methyl)-1-methylcyclopropane-1-carboxamide

To a solution of N-((6-bromo-5-(difluoromethoxy)-1-(phenylsulfonyl)-1H-indol-2-yl)methyl)-1-methylcyclopropane-1-carboxamide (650 mg, 0.59 mmol, 1.00 eq) in dioxane (3 mL) and water (0.6 mL) were added Cs₂CO₃ (254 mg, 0.78 mmol, 1.00 eq) and KOH (87.4 mg, 1.56 mmol, 4.00 eq). The mixture was degassed and purged with N₂ three times. Pd₂(dba)₃ (35.7 mg, 0.039 mmol, 0.1 eq) and tBuXPhos (16.5 mg, 0.039 mmol, 0.1 eq) were added and the reaction was heated to 120° C. and stirred for 1 h. The reaction mixture was cooled to room temperature and poured into water (15 mL). The resulting mixture was extracted with EtOAc (3×15 mL). The combined organic layers were washed with brine (2×15 mL), dried over anhydrous Na₂SO₄, filtered, and concentrated under reduced pressure. Purification by column chromatography gave N-((5-(difluoromethoxy)-6-hydroxy-1-(phenylsulfonyl)-1H-indol-2-yl)methyl)-1-methylcyclopropane-1-carboxamide. LC-MS (ESI) m/z calcd for C₂₁H₂₀F₂N₂O₅S: 450.1; found: 451.1 [M+H]⁺.

### Step 6: N-((5-(difluoromethoxy)-1-(phenylsulfonyl)-6-(thiazol-4-ylmethoxy)-1H-indol-2-yl)methyl)-1-methylcyclopropane-1-carboxamide

To a solution of N-((5-(difluoromethoxy)-6-hydroxy-1-(phenylsulfonyl)-1H-indol-2-yl)methyl)-1-methylcyclopropane-1-carboxamide (100 mg, 0.22 mmol, 1.00 eq) in DMF (0.8 mL) were added a solution of 4-(chloromethyl)thiazole hydrochloride (38.5 mg, 0.29 mmol, 1.30 eq) and DIEA (155 μL, 0.89 mmol, 4.00 eq) in DMF (0.2 mL) dropwise. KI (3.7 mg, 22.2 μmol, 0.1 eq) and Cs₂CO₃ (145 mg, 0.044 mmol, 4.00 eq) were added and the reaction was heated to 70° C. for 2 h. The reaction mixture was cooled to room temperature and poured into water (10 mL). The resulting mixture was extracted with EtOAc (3×15 mL). The combined organic layers were washed with brine (20 mL), dried over anhydrous Na₂SO₄, filtered, and concentrated under reduced pressure. Purification by column chromatography gave N-((5-(difluoromethoxy)-1-(phenylsulfonyl)-6-(thiazol-4-ylmethoxy)-1H-indol-2-yl)methyl)-1-methylcyclopropane-1-carboxamide. LC-MS (ESI) m/z calcd for C₂₅H₂₃F₂N₃O₅S₂: 547.1; found: 548.2 [M+H]⁺.

### Step 7: N-((5-(difluoromethoxy)-6-(thiazol-4-ylmethoxy)-1H-indol-2-yl)methyl)-1-methylcyclopropane-1-carboxamide

A solution of TBAF (1 M in THF, 511 μL, 0.65 mmol, 5.00 eq) was added to N-((5-(difluoromethoxy)-1-(phenylsulfonyl)-6-(thiazol-4-ylmethoxy)-1H-indol-2-yl)methyl)-1-methylcyclopropane-1-carboxamide (70 mg, 0.13 mmol, 1.00 eq) and the reaction was heated to 50° C. for 2 h. The reaction mixture was cooled to room temperature and poured into water (10 mL). The resulting mixture was extracted with EtOAc (3×15 mL). The combined organic layers were washed with sat. aq. NH₄Cl (3×15 mL), dried over anhydrous Na₂SO₄, filtered, and concentrated under reduced pressure. Purification by prep-HPLC gave N-((5-(difluoromethoxy)-6-(thiazol-4-ylmethoxy)-1H-indol-2-yl)methyl)-1-methylcyclopropane-1-carboxamide (Compound 604). LC-MS (ESI) m/z calcd for C₁₉H₁₉F₂N₃O₃S: 407.11; found: 408.2 [M+H]⁺. ¹H NMR (400 MHz, DMSO-d₆) δ 10.7 (s, 1H), 9.14 (d, J=1.6 Hz, 1H), 8.00 (t, J=5.2 Hz, 1H), 7.73 (s, 1H), 7.25 (s, 1H), 7.17 (s, 1H), 6.94 (t, J=76 Hz, 1H), 6.14 (s, 1H), 5.27 (s, 2H), 4.34 (d, J=5.6 Hz, 2H), 1.28 (s, 3H), 1.00-0.98 (m, 2H), 0.54-0.51 (m, 2H).

### Example 60 (Compound 605)

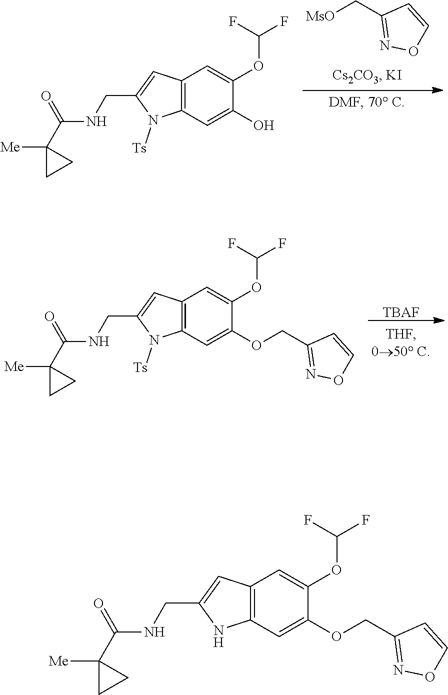 [SMILES: Cc1ccc(S(=O)(=O)n2c(CNC(=O)C3(C)CC3)cc3cc(OC(F)F)c(O)cc32)cc1 ; CS(=O)(=O)OCc1ccon1 ; Cc1ccc(S(=O)(=O)n2c(CNC(=O)C3(C)CC3)cc3cc(OC(F)F)c(OCc4ccon4)cc32)cc1 ; "TBAF" ; CC1(C(=O)NCc2cc3cc(OC(F)F)c(OCc4ccon4)cc3[nH]2)CC1]

### Step 1: N-((5-(difluoromethoxy)-6-(isoxazol-3-ylmethoxy)-1-tosyl-1H-indol-2-yl)methyl)-1-methylcyclopropane-1-carboxamide

To a mixture of isoxazol-3-ylmethyl methanesulfonate (91.5 mg, 517 μmol, 1.20 eq) and N-((5-(difluoromethoxy)-6-hydroxy-1-tosyl-1H-indol-2-yl) methyl)-1-methylcyclopropane-1-carboxamide (200 mg, 431 μmol, 1.00 eq) in DMF (2 mL) was added KI (7.15 mg, 43.1 μmol, 0.1 eq) and Cs₂CO₃ (281 mg, 861 μmol, 2.00 eq). The reaction mixture was then heated to 70° C. and stirred 1 h. After cooling to room temperature, the reaction mixture was poured into water (20 mL). The aqueous phase was extracted with ethyl acetate (3×10 mL). The combined organic phase was washed with brine (2×10 mL), dried over anhydrous Na₂SO₄, filtered, and concentrated under reduced pressure. The resulting residue was purified by prep-TLC to give N-((5-(difluoromethoxy)-6-(isoxazol-3-ylmethoxy)-1-tosyl-1H-indol-2-yl) methyl)-1-methylcyclopropane-1-carboxamide. LC-MS (ESI) m/z calcd for C₂₆H₂₅F₂N₃O₆S: 545.14; found: 546.3 [M+H]⁺.

### Step 2: N-((5-(difluoromethoxy)-6-(isoxazol-3-ylmethoxy)-1H-indol-2-yl)methyl)-1-methylcyclopropane-1-carboxamide

To a solution of N-((5-(difluoromethoxy)-6-(isoxazol-3-ylmethoxy)-1-tosyl-1H-indol-2-yl) methyl)-1-methylcyclopropane-1-carboxamide (150 mg, 275 μmol, 1.00 eq) in THF (0.2 mL) was added TBAF (1 M in THF, 1.37 mL, 5.00 eq) in portions at 0° C. The reaction mixture was then heated to 50° C. and stirred for 1 h. After cooling to room temperature, the reaction mixture was poured into water (15 mL). The aqueous phase was extracted with ethyl acetate (3×15 mL). The combined organic phase was washed with sat. aq. NH₄Cl solution (3×15 mL), dried over anhydrous Na₂SO₄, filtered, and concentrated under reduced pressure. Purification by prep-HPLC gave N-((5-(difluoromethoxy)-6-(isoxazol-3-ylmethoxy)-1H-indol-2-yl) methyl)-1-methylcyclopropane-1-carboxamide (Compound 605). LC-MS (ESI) m/z calcd for C₁₉H₁₉F₂N₃O₄: 391.13; found 392.2 [M+H]⁺. ¹H NMR (400 MHz, DMSO-d₆) δ 10.79 (s, 1H), 8.95 (s, 1H), 8.00 (t, J=5.8 Hz, 1H), 7.26 (s, 1H), 7.16 (s, 1H), 7.12-6.74 (t, J=75.6 Hz, 1H), 6.69 (d, J=1.2 Hz, 1H), 6.15 (s, 1H), 5.26 (s, 2H), 4.35 (d, J=5.6 Hz, 2H), 1.28 (s, 3H), 1.00-0.98 (m, 2H), 0.54-0.51 (m, 2H).

### Example 61 (Compound 606)

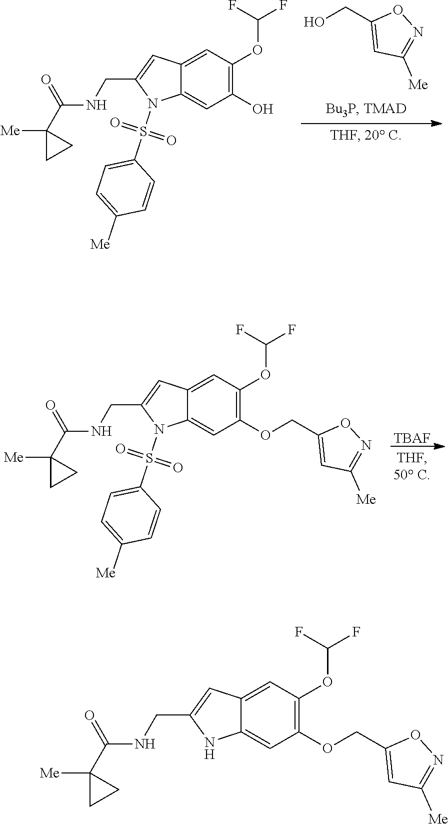

### Step 1: N-((5-(difluoromethoxy)-6-((3-methylisoxazol-5-yl)methoxy)-1-tosyl-1H-indol-2-yl)methyl)-1-methylcyclopropane-1-carboxamide

To a solution of (3-methylisoxazol-5-yl) methanol (73.0 mg, 646 μmol, 2.00 eq) and N-((5-(difluoromethoxy)-6-hydroxy-1-tosyl-1H-indol-2-yl)methyl)-1-methylcyclopropane-1-carboxamide (150 mg, 323 μmol, 1.00 eq) in THF (2 mL) was added N,N,N′,N-tetramethyldiazene-1,2-dicarboxamide (TMAD, 83.4 mg, 484 μmol, 1.50 eq) and tributylphosphine (Bu₃P, 484 μmol, 120 L, 1.50 eq) in portions. The reaction mixture was degassed and purged with N₂ three times and then stirred at 20° C. for 1 h under an N₂ atmosphere. The reaction mixture was poured into water (10 mL). The aqueous phase was extracted with ethyl acetate (3×10 mL). The organic phase was washed with brine (10 mL), dried over anhydrous Na₂SO₄, filtered, and concentrated under reduced pressure. Purification by prep-TLC gave N-((5-(difluoromethoxy)-6-((3-methylisoxazol-5-yl)methoxy)-1-tosyl-1H-indol-2-yl) methyl)-1-methylcyclopropane-1-carboxamide. LC-MS (ESI) m/z calcd for C₂₇H₂₇F₂N₃O₆S: 559.16; found: 560.2 [M+H]⁺.

### Step 2: N-((5-(difluoromethoxy)-6-((3-methylisoxazol-5-yl)methoxy)-1H-indol-2-yl)methyl)-1-methylcyclopropane-1-carboxamide

To a solution of N-((5-(difluoromethoxy)-6-((3-methylisoxazol-5-yl)methoxy)-1-tosyl-1H-indol-2-yl) methyl)-1-methylcyclopropane-1-carboxamide (130 mg, 232 μmol, 1.00 eq) in THF (0.2 mL) was added TBAF (1 M in THF, 1.16 mL, 5.00 eq) dropwise at 0° C. The reaction mixture was warmed to 50° C. and stirred for 1 h. The reaction mixture was poured into water (10 mL) and the aqueous phase was extracted with ethyl acetate (2×10 mL). The combined organic phase was washed with sat. aq. NH₄Cl solution (3×10 mL), dried with anhydrous Na₂SO₄, filtered, and concentrated under reduced pressure. Purification by prep-HPLC gave N-((5-(difluoromethoxy)-6-((3-methylisoxazol-5-yl)methoxy)-1H-indol-2-yl)methyl)-1-methylcyclopropane-1-carboxamide (Compound 606). LC-MS (ESI) m/z calcd for C₂₀H₂₁F₂N₃O₄: 405.15; found: 406.3 [M+H]⁺. ¹H NMR (400 MHz, DMSO-d₆) δ 10.77 (s, 1H), 8.01 (t, J=5.6 Hz, 1H), 7.26 (s, 1H), 7.14 (s, 1H), 6.90 (t, J=75.2 Hz, 1H), 6.44 (s, 1H), 6.16 (s, 1H), 5.25 (s, 2H), 4.35 (d, J=6.0 Hz, 2H), 2.23 (s, 3H), 1.28 (s, 3H), 1.00-0.98 (m, 2H), 0.54-0.51 (m, 2H).

### Example 62 (Compound 607)

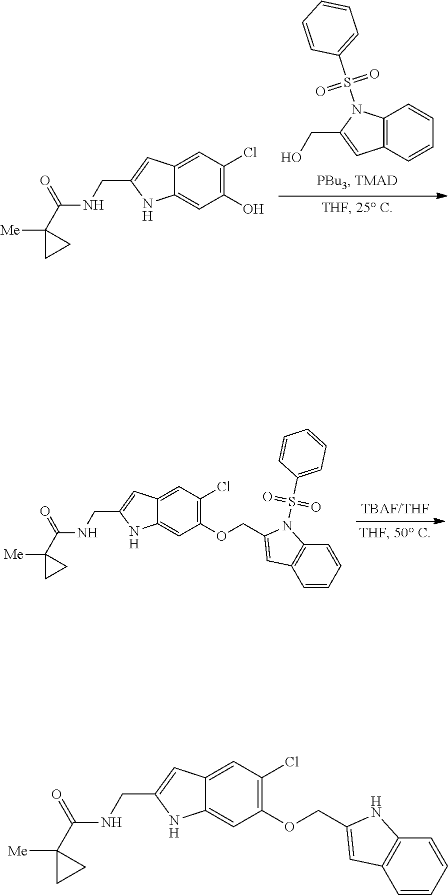 [SMILES: CC1(C(=O)NCc2cc3cc(Cl)c(O)cc3[nH]2)CC1 ; O=S(=O)(c1ccccc1)n1c(CO)cc2ccccc21 ; CC1(C(=O)NCc2cc3cc(Cl)c(OCc4cc5ccccc5n4S(=O)(=O)c4ccccc4)cc3[nH]2)CC1 ; CC1(C(=O)NCc2cc3cc(Cl)c(OCc4cc5ccccc5[nH]4)cc3[nH]2)CC1]

### Step 1: N-((5-chloro-6-((1-(phenylsulfonyl)-1H-indol-2-yl)methoxy)-1H-indol-2-yl)methyl)-1-methylcyclopropane-1-carboxamide

To a solution of N-((5-chloro-6-hydroxy-1H-indol-2-yl)methyl)-1-methylcyclopropane-1-carboxamide (200 mg, 717 μmol, 1.00 eq) in THF (2 mL) was added (1-(phenylsulfonyl)-1H-indol-2-yl)methanol (247 mg, 861 μmol, 1.20 eq) and TMAD (370 mg, 2.15 mmol, 3.00 eq) in one portion. The mixture was degassed and purged with N₂ three times, and then tributylphosphane (435 mg, 2.15 mmol, 3.00 eq) was added dropwise. After 12 h, the reaction was quenched by addition of H₂O (5 mL). The solution was extracted with ethyl acetate (3×10 mL). The combined organic layers were washed with brine (5 mL), dried over anhydrous Na₂SO₄, filtered, and concentrated under reduced pressure. Purification by column chromatography gave N-((5-chloro-6-((1-(phenylsulfonyl)-1H-indol-2-yl)methoxy)-1H-indol-2-yl)methyl)-1-methylcyclopropane-1-carboxamide. LC-MS (ESI) m/z calcd for C₂₉H₂₆ClN₃O₄S: 547.1; found 548.3 [M+H]⁺.

### Step 2: N-((6-((1H-indol-2-yl)methoxy)-5-chloro-1H-indol-2-yl)methyl)-1-methylcyclopropane-1-carboxamide

To a solution of N-((5-chloro-6-((1-(phenylsulfonyl)-1H-indol-2-yl)methoxy)-1H-indol-2-yl)methyl)-1-methylcyclopropane-1-carboxamide (110 mg, 200 μmol, 1.00 eq) in THF (0.5 mL) was added TBAF (1 M in THF, 1.00 mL, 5.00 eq) dropwise. The reaction mixture was heated to 50° C. After 2 h, the reaction was quenched by addition of H₂O (5 mL). The solution was extracted with ethyl acetate (3×10 mL). The combined organic layers were washed with brine (5 mL), dried over anhydrous Na₂SO₄, filtered, and concentrated under reduced pressure. Purification by prep-HPLC gave N-((6-((1H-indol-2-yl)methoxy)-5-chloro-1H-indol-2-yl)methyl)-1-methylcyclopropane-1-carboxamide (Compound 607). LC-MS (ESI) m/z calcd for C₂₃H₂₂ClN₃O₂: 407.1; found 408.1 [M+H]⁺. ¹H NMR (400 MHz, DMSO-d₆) δ 11.32 (s, 1H), 10.75 (s, 1H), 8.00 (t, J=6.0 Hz, 1H), 7.51 (d, J=8.0 Hz, 1H), 7.49 (s, 1H), 7.37 (d, J=8.0 Hz, 1H), 7.21 (s, 1H), 7.07 (t, J=6.8 Hz, 1H), 6.98 (t, J=8.0 Hz, 1H), 6.52 (s, 1H), 6.11 (s, 1H), 5.23 (s, 2H), 4.35 (d, J=5.6 Hz, 2H), 1.28 (s, 3H), 1.00-0.98 (m, 2H), 0.54-0.52 (m, 2H).

### Example 63 (Compound 608)

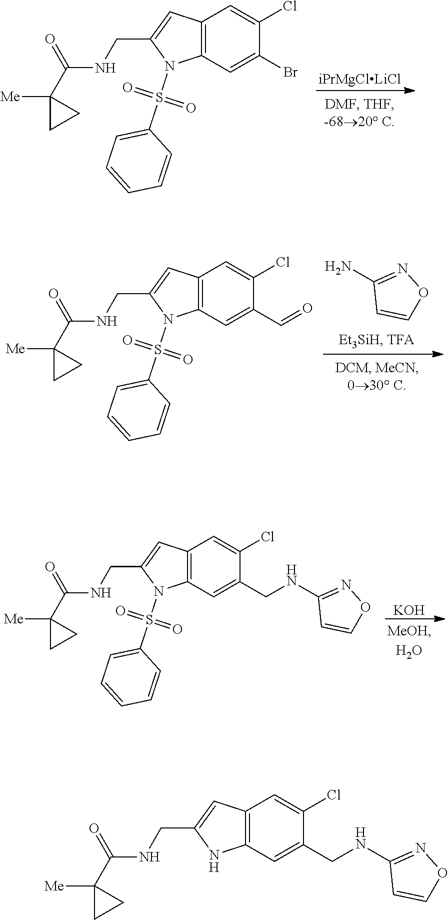

### Step 1: N-((5-chloro-6-formyl-1-(phenylsulfonyl)-1H-indol-2-yl)methyl)-1-methylcyclopropane-1-carboxamide

A solution of N-((6-bromo-5-chloro-1-(phenylsulfonyl)-1H-indol-2-yl)methyl)-1-methylcyclopropane-1-carboxamide (25.0 g, 51.8 mmol, 1.00 eq) in THF (250 mL) was degassed and purged with N₂ three times and then cooled to −68° C. i-PrMgCl—LiCl (1.30 M in THF, 159 mL, 4.00 eq) was added dropwise at −68° C. under N₂ and then reaction was allowed to warm to 20° C. After 0.5 h, the resulting mixture was cooled to 0° C. and DMF (39.9 mL, 518 mmol, 10.0 eq) was added dropwise. The reaction was allowed to warm to 20° C. and stirred for 1 h. The mixture was adjusted to pH 7 by addition of aq. HCl (1M) then extracted with EtOAc (3×300 mL). The combined organic layers were washed with brine (300 mL), dried over Na₂SO₄, filtered, and concentrated under reduced pressure to give a residue. The residue was triturated with MTBE (50 mL) for 30 mins and then filtered, and the filter cake was washed with MTBE (50 mL) and dried under reduced pressure to give N-((5-chloro-6-formyl-1-(phenylsulfonyl)-1H-indol-2-yl)methyl)-1-methylcyclopropane-1-carboxamide. LC-MS (ESI) m/z calcd for C21H₁₉ClN₂O₄S: 430.08; found 431.2 [M+H]⁺.

### Step 2: N-((5-chloro-6-((isoxazol-3-ylamino)methyl)-1-(phenylsulfonyl)-1H-indol-2-yl)methyl)-1-methylcyclopropane-1-carboxamide

Two reactions were carried out in parallel. To a mixture of N-((5-chloro-6-formyl-1-(phenylsulfonyl)-1H-indol-2-yl)methyl)-1-methylcyclopropane-1-carboxamide (36.0 g, 83.5 mmol, 1.00 eq) and isoxazol-3-amine (24.5 g, 292 mmol, 21.6 mL, 3.50 eq) in DCM (50 mL) and MeCN (150 mL) at 0° C. was added dropwise Et₃SiH (46.7 mL, 292 mmol, 3.50 eq) and TFA (12.4 mL, 167 mmol, 2.00 eq). The reaction was heated to 30° C. and stirred for 16 h. The two reactions were combined for work up. The reaction was cooled to 20° C. and adjusted to pH 7 with sat. aq. NaHCO₃ solution then extracted with EtOAc (3×300 mL). The combined organic phases was washed with brine (300 mL), dried over Na₂SO₄, filtered, and concentrated under reduced pressure. The resulting residue was triturated with MTBE (100 mL) for 30 mins at 50° C. and filtered at 50° C. The filter cake was washed with MTBE (100 mL) at 50° C. and dried under reduced pressure to give N-((5-chloro-6-((isoxazol-3-ylamino)methyl)-1-(phenylsulfonyl)-1H-indol-2-yl)methyl)-1-methylcyclopropane-1-carboxamide. LC-MS (ESI) m/z calcd for C₂₄H₂₃ClN₄O₄S: 498.11; found 499.3 [M+H]⁺.

### Step 3: N-((5-chloro-6-((isoxazol-3-ylamino)methyl)-1H-indol-2-yl)methyl)-1-methylcyclopropane-1-carboxamide

To a solution of N-((5-chloro-6-((isoxazol-3-ylamino)methyl)-1-(phenylsulfonyl)-1H-indol-2-yl)methyl)-1-methylcyclopropane-1-carboxamide (45.0 g, 90.1 mmol, 1.00 eq) in MeOH (450 mL) and H₂O (150 mL) was added KOH (15.1 g, 270 mmol, 3.00 eq). The resulting mixture was heated to 80° C. After 2 h, the reaction was cooled to room temperature and filtered. The filter cake was diluted with H₂O (500 mL) and extracted with EtOAc (3×500 mL). The combined organic layers were washed with brine (500 mL), dried over anhydrous Na₂SO₄, filtered, and concentrated under reduced pressure. The resulting residue was triturated with H₂O (100 mL) for 30 mins at 80° C. then filtered at 80° C. The filter cake was washed with H₂O (100 mL) and dried under reduced pressure to give N-((5-chloro-6-((isoxazol-3-ylamino)methyl)-1H-indol-2-yl)methyl)-1-methylcyclopropane-1-carboxamide (Compound 608). LC-MS (ESI) m/z calcd for C₁₈H₁₉ClN₄O₂: 358.12; found 359.2 [M+H]⁺. ¹H NMR (400 MHz, DMSO-d₆) δ 10.96 (s, 1H), 8.36 (d, J=2.0 Hz, 1H), 8.03 (t, J=5.6 Hz, 1H), 7.51 (s, 1H), 7.41 (s, 1H), 6.63 (t, J=6.0 Hz, 1H), 6.17 (s, 1H), 6.00 (d, J=2.0 Hz, 1H), 4.38 (d, J=5.6 Hz, 4H), 1.29 (s, 3H), 1.00-0.98 (m, 2H), 0.54-0.52 (m, 2H).

### Example 64 (Compound 609)

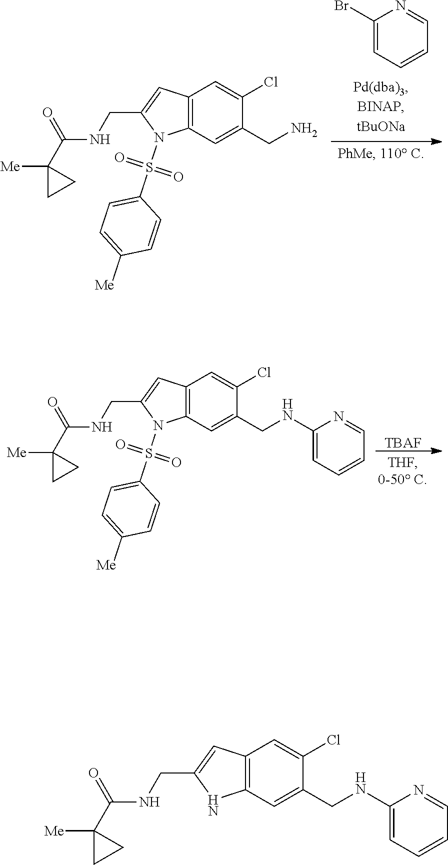

### Step 1: N-((5-chloro-6-((pyridin-2-ylamino)methyl)-1-tosyl-1H-indol-2-yl)methyl)-1-methylcyclopropane-1-carboxamide

To a solution of N-((6-(aminomethyl)-5-chloro-1-tosyl-1H-indol-2-yl)methyl)-1-methylcyclopropane-1-carboxamide (1.00 g, 2.24 mmol, 1.00 eq) in PhMe (5 mL) were added sodium tert-butoxide (302 mg, 3.14 mmol, 1.40 eq) and 2-bromopyridine (709 mg, 4.48 mmol, 2.00 eq). The mixture was degassed and purged with N₂ three times. Pd₂(dba)₃ (205 mg, 224 μmol, 0.10 eq) and BINAP (279 mg, 448 μmol, 0.2 eq) were added and the resulting reaction mixture was heated to 110° C. for 16 h. The reaction mixture was cooled to 25° C. and quenched with water (15 mL) and filtered. The filtrate was extracted with EtOAc (3×15 mL). The combined organic layers were washed with brine (15 mL), dried over anhydrous Na₂SO₄, filtered, and concentrated under reduced pressure. Purification with column chromatography gave N-((5-chloro-6-((pyridin-2-ylamino)methyl)-1-tosyl-1H-indol-2-yl)methyl)-1-methylcyclopropane-1-carboxamide. LC-MS (ESI) m/z calcd for C₂₇H₂₇ClN₄O₃S: 522.15; found: 523.2 [M+H]⁺.

### Step 2: N-((5-chloro-6-((pyridin-2-ylamino)methyl)-1H-indol-2-yl)methyl)-1-methylcyclopropane-1-carboxamide

To a solution of N-((5-chloro-6-((pyridin-2-ylamino)methyl)-1-tosyl-1H-indol-2-yl)methyl)-1-methylcyclopropane-1-carboxamide (2.80 g, 5.13 mmol, 1.00 eq) in THF (5 mL) was added TBAF (1 M in THF, 1.34 mL, 1.00 eq) at 0° C. The resulting reaction mixture was heated to 50° C. for 2 h. The reaction mixture was then quenched with the addition of water (15 mL), and the resulting mixture was extracted with EtOAc (3×30 mL). The combined organic layers were washed with brine (15 mL), dried over anhydrous Na₂SO₄, filtered, and concentrated under reduced pressure. Purification by prep-TLC gave N-((5-chloro-6-((pyridin-2-ylamino)methyl)-1H-indol-2-yl)methyl)-1-methylcyclopropane-1-carboxamide (Compound 609). LC-MS (ESI) m/z calcd for C₂₀H₂₁ClN₄O: 368.14; found: 369.1 [M+H]⁺. ¹H NMR (400 MHz, DMSO-d₆) δ 10.88 (s, 1H), 8.01 (t, J=17.2 Hz, 1H), 7.93 (d, J=4.0 Hz, 1H), 7.49 (s, 1H), 7.39-7.36 (m, 1H), 7.33 (s, 1H), 7.00-7.03 (m, 1H), 6.54 (d, J=8.4 Hz, 1H), 6.47 (t, J=2.8 Hz, 1H), 6.15 (s, 1H), 4.55 (d, J=5.6 Hz, 2H), 4.35 (d, J=5.6 Hz, 2H), 1.27 (s, 3H), 0.99-0.96 (m, 2H), 0.53-0.50 (m, 2H).

The following compounds in Table T-37 were synthesized using procedures similar to Compound 609 using the appropriate starting materials.

TABLE T-37

| Com- | | | Exact | LCMS, |
|---|---|---|---|---|
| pound | | | mass | found |
| No. | Structure | IUPAC name | (g/mol) | [M + H]⁺ |
| 610 | 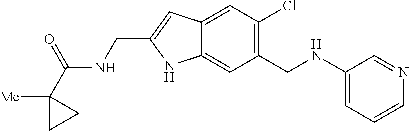 [SMILES: CC1(C(=O)NCc2cc3cc(Cl)c(CNc4cccnc4)cc3[nH]2)CC1] | N-((5-chloro-6- ((pyridin-3- ylamino)methyl)-1H- indol-2-yl)methyl)-1- methylcyclopropane-1- carboxamide | 368.14 | 369.1 |

### Example 65 (Compound 611)

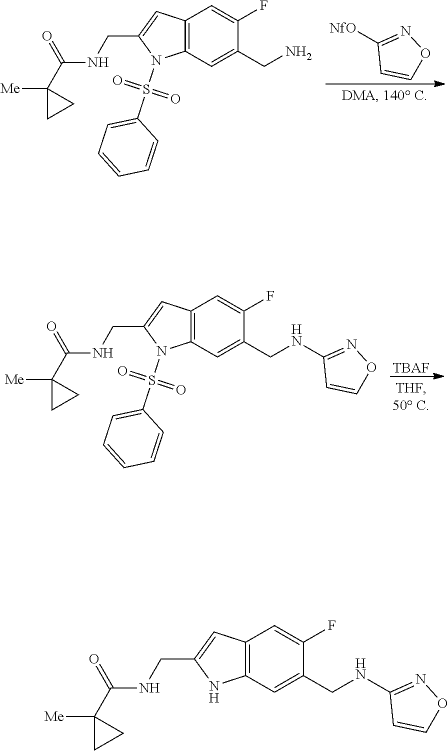

### Step 1: N-((5-fluoro-6-((isoxazol-3-ylamino)methyl)-1-(phenylsulfonyl)-1H-indol-2-yl)methyl)-1-methylcyclopropane-1-carboxamide

To a solution of N-((6-(aminomethyl)-5-fluoro-1-(phenylsulfonyl)-1H-indol-2-yl)methyl)-1-methylcyclopropane-1-carboxamide (452 mg, 1.09 mmol, 1.00 eq) in DMA (10 mL) was added isoxazol-3-yl 1,1,2,2,3,3,4,4,4-nonafluorobutane-1-sulfonate (1.91 g, 5.45 mmol, 5.00 eq) in one portion. The reaction mixture was heated and stirred at 140° C. for 3 h. The reaction mixture was quenched by addition of H₂O (10 mL) at 20° C. and extracted with ethyl acetate (2×20 mL). The combined organic layers were washed with H₂O (2×30 mL), dried over Na₂SO₄, filtered, and concentrated under reduced pressure to give a residue. The residue was purified by column chromatography to give N-((5-fluoro-6-((isoxazol-3-ylamino)methyl)-1-(phenylsulfonyl)-1H-indol-2-yl)methyl)-1-methylcyclopropane-1-carboxamide. LC-MS (ESI) m/z calcd for C₂₄H₂₃FN₄O₄S: 482.14; found: 483.3 [M+H]⁺.

### Step 2: N-((5-fluoro-6-((isoxazol-3-ylamino)methyl)-1H-indol-2-yl)methyl)-1-methylcyclopropane-1-carboxamide

To a solution of N-((5-fluoro-6-((isoxazol-3-ylamino)methyl)-1-(phenylsulfonyl)-1H-indol-2-yl)methyl)-1-methylcyclopropane-1-carboxamide (50.0 mg, 103 μmol, 1.00 eq) in THF (0.1 mL) was added TBAF (1 M in THF, 310 μL, 3.00 eq) in one portion. The reaction mixture was heated and stirred at 50° C. for 12 h. The reaction mixture was quenched by addition of H₂O (10 mL) at 20° C., extracted with ethyl acetate (2×5 mL). The combined organic layers were washed with brine (2×5 mL), dried over Na₂SO₄, filtered, and concentrated under reduced pressure to give a residue. The residue was purified by prep-TLC to give N-((5-fluoro-6-((isoxazol-3-ylamino)methyl)-1H-indol-2-yl)methyl)-1-methylcyclopropane-1-carboxamide (Compound 611). LC-MS (ESI) m/z calcd for C₁₈H₁₉FN₄O₂: 342.15; found: 343.1 [M+H]⁺. ¹H NMR (400 MHz, DMSO-d₆) δ 10.86 (s, 1H), 8.35 (d, J=1.6 Hz, 1H), 8.03-8.01 (m, 1H), 7.33 (d, J=6.4 Hz, 1H), 7.18 (d, J=11.2 Hz, 1H), 6.59-6.57 (m, 1H), 6.16 (s, 1H), 5.97 (d, J=2.0 Hz, 1H), 4.37 (d, J=5.6 Hz, 2H), 4.33 (d, J=6.0 Hz, 2H), 1.28 (s, 3H), 1.00-0.97 (m, 2H), 0.53-0.51 (m, 2H).

### Example 66 (Compound 612)

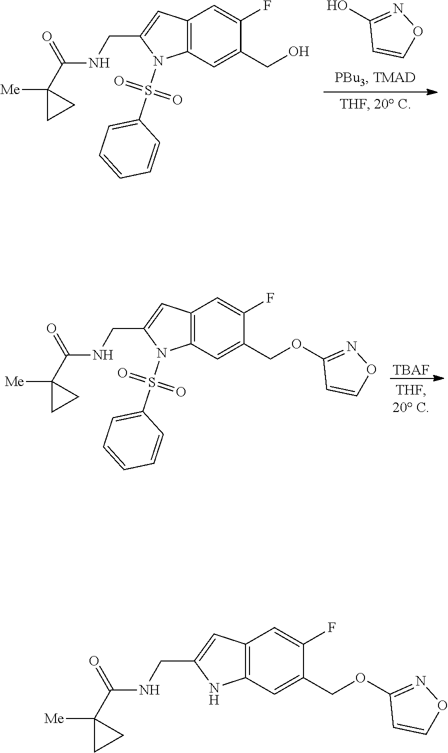

### Step 1: N-((5-fluoro-6-((isoxazol-3-yloxy)methyl)-1-(phenylsulfonyl)-1H-indol-2-yl)methyl)-1-methylcyclopropane-1-carboxamide

A mixture of N-((5-fluoro-6-(hydroxymethyl)-1-(phenylsulfonyl)-1H-indol-2-yl)methyl)-1-methylcyclopropane-1-carboxamide (100 mg, 240 μmol, 1.00 eq) and isoxazol-3-ol (40.0 mg, 480 μmol, 2.00 eq) in THF (3 mL) was degassed under vacuum and purged with N₂ three times. Tributylphosphine (1.20 mmol, 296 uL, 5.00 eq) and N,N,N′,N′-tetramethylazodicarboxamide (206 mg, 1.20 mmol, 5.00 eq) were added and the resulting mixture was stirred under N₂ at 20° C. After 3 h, H₂O (10 mL) was added and the reaction was extracted with ethyl acetate (2×5 mL). The combined organic phase was washed with brine (10 mL), dried over anhydrous Na₂SO₄, filtered, and concentrated under reduced pressure. The residue was purified by prep-TLC to give N-((5-fluoro-6-((isoxazol-3-yloxy)methyl)-1-(phenylsulfonyl)-1H-indol-2-yl)methyl)-1-methylcyclopropane-1-carboxamide. LC-MS (ESI) m/z calcd for C₂₄H₂₂FN₃O₅S: 483.13; found 506.2 [M+Na]⁺.

### Step 2: N-((5-fluoro-6-((isoxazol-3-yloxy)methyl)-1H-indol-2-yl)methyl)-1-methylcyclopropane-1-carboxamide

To a solution of N-((5-fluoro-6-((isoxazol-3-yloxy)methyl)-1-(phenylsulfonyl)-1H-indol-2-yl)methyl)-1-methylcyclopropane-1-carboxamide (50.0 mg, 103 μmol, 1.00 eq) in THF (0.5 mL) at 20° C. was added TBAF (1 M in THF, 517 μL, 5.00 eq) in portions. The reaction mixture was stirred at 20° C. After 24 h, the reaction mixture was poured into H₂O (5 mL). The aqueous phase was extracted with ethyl acetate (2×5 mL). The combined organic phase was washed with sat. aq. NH₄Cl (2×10 mL), dried over anhydrous Na₂SO₄, filtered, and concentrated under reduced pressure. The resulting residue was purified by prep-HPLC to give N-((5-fluoro-6-((isoxazol-3-yloxy)methyl)-1H-indol-2-yl)methyl)-1-methylcyclopropane-1-carboxamide (Compound 612). LC-MS (ESI) m/z calcd for C₁₈H₁₈FN₃O₃: 343.13; found 366.1 [M+Na]⁺. ¹H NMR (400 MHz, DMSO-d₆) δ 11.02 (s, 1H), 8.66 (d, J=2.0 Hz, 1H), 8.06 (t, J=5.6 Hz, 1H), 7.50 (d, J=6.0 Hz, 1H), 7.26 (d, J=11.2 Hz, 1H), 6.34 (d, J=1.6 Hz, 1H), 6.22 (s, 1H), 5.31 (s, 2H), 4.39 (d, J=5.6 Hz, 2H), 1.30 (s, 3H), 1.01-0.98 (m, 2H), 0.55-0.52 (m, 2H).

The following compounds in Table T-38 were synthesized using procedures similar to Compound 612 using the appropriate starting materials.

TABLE T-38

| Com- | | | Exact | LCMS, |
|---|---|---|---|---|
| pound | | | mass | found |
| No. | Structure | IUPAC name | (g/mol) | [M + H]⁺ |
| 613 | 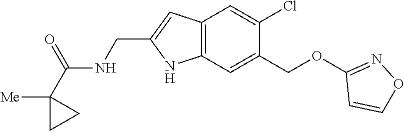 [SMILES: CC1(C(=O)NCc2cc3cc(Cl)c(COc4ccon4)cc3[nH]2)CC1] | N-((5-chloro-6- ((isoxazol-3- yloxy)methyl)-1H- indol-2-yl)methyl)-1- methylcyclopropane-1- carboxamide | 359.10 | 382.1 |

### Example 67 (Compound 614)

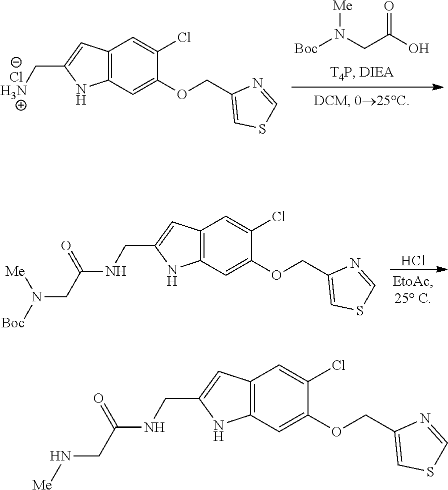 [SMILES: [Cl-] ; [NH3+]Cc1cc2cc(Cl)c(OCc3cscn3)cc2[nH]1 ; CN(CC(=O)O)C(=O)OC(C)(C)C ; CN(CC(=O)NCc1cc2cc(Cl)c(OCc3cscn3)cc2[nH]1)C(=O)OC(C)(C)C ; Cl ; CNCC(=O)NCc1cc2cc(Cl)c(OCc3cscn3)cc2[nH]1]

### Step 1: tert-butyl (2-(((5-chloro-6-(thiazol-4-ylmethoxy)-1H-indol-2-yl)methyl)amino)-2-oxoethyl)(methyl)carbamate

A mixture of (5-chloro-6-(thiazol-4-ylmethoxy)-1H-indol-2-yl)methanamine hydrochloride (300 mg, 908 μmol, 1.00 eq) and N-(tert-butoxycarbonyl)-N-methylglycine (24.0 mg, 1.27 mmol, 1.40 eq) in DCM (6 mL) were added DIEA (234 mg, 1.82 mmol, 2.00 eq) and 2,4,6-tributyl-1,3,5,2,4,6-trioxatriphosphinane-2,4,6-trioxide (T₄P, 785 mg, 1.09 mmol, 1.20 eq) dropwise at 0° C. After addition, the reaction mixture was allowed to warm to 25° C. and stirred for 2 h. The reaction mixture was quenched with H₂O (30 mL), and the aqueous phase was extracted with DCM (3×20 mL). The combined organic phase was washed with brine (2×20 mL), dried over Na₂SO₄, filtered, and concentrated under reduced pressure. Purification by column chromatography gave tert-butyl (2-(((5-chloro-6-(thiazol-4-ylmethoxy)-1H-indol-2-yl) methyl) amino)-2-oxoethyl) (methyl) carbamate. LC-MS (ESI) m/z calcd for C₂₁H₂₅ClN₄O₄S: 464.13; found: 465.2 [M+H]⁺.

### Step 2: N-((5-chloro-6-(thiazol-4-ylmethoxy)-1H-indol-2-yl)methyl)-2-(methylamino)acetamide

To a solution of tert-butyl (2-(((5-chloro-6-(thiazol-4-ylmethoxy)-1H-indol-2-yl) methyl) amino)-2-oxoethyl) (methyl) carbamate (180 mg, 387 μmol, 1.00 eq) in EtOAc (3 mL) was added HCl (4 M in EtOAc, 6 mL, 62.0 eq) dropwise at 25° C. The reaction mixture was stirred for 1 h. The reaction mixture was concentrated in vacuo. Purification by prep-HPLC gave N-((5-chloro-6-(thiazol-4-ylmethoxy)-1H-indol-2-yl) methyl)-2-(methylamino) acetamide (Compound 614). LC-MS (ESI) m/z calcd for C₁₆H₁₇ClN₄O₂S: 364.08; found: 365.1 [M+H]⁺. ¹H NMR (400 MHz, DMSO-d₆) δ 10.91 (s, 1H), 9.14 (s, 1H), 8.23 (t, J=5.6 Hz, 1H), 7.75 (d, J=1.6 Hz, 1H), 7.49 (s, 1H), 7.17 (s, 1H), 6.15 (s, 1H), 5.27 (s, 2H), 4.38 (d, J=5.2 Hz, 2H), 3.08 (s, 2H), 2.26 (s, 3H).

The following compounds in Table T-39 were synthesized using procedures similar to Compound 614 using the appropriate starting materials.

TABLE T-39

| Com- | | | Exact | LCMS, |
|---|---|---|---|---|
| pound | | | mass | found |
| No. | Structure | IUPAC name | (g/mol) | [M + H]⁺ |
| 615 | 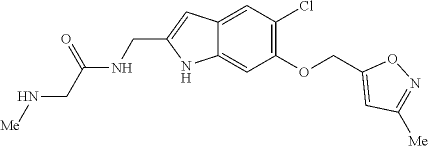 [SMILES: CNCC(=O)NCc1cc2cc(Cl)c(OCc3cc(C)no3)cc2[nH]1] | N-((5-chloro-6-((3- methylisoxazol-5- yl)methoxy)-1H-indol- 2-yl)methyl)-2- (methylamino)acetamide | 362.11 | 363.2 |
| 616 | 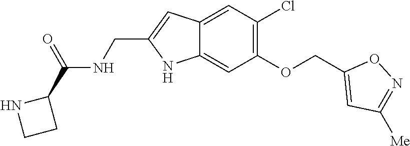 [SMILES: Cc1cc(COc2cc3[nH]c(CNC(=O)[C@H]4CCN4)cc3cc2Cl)on1] | (R)-N-((5-chloro-6-((3- methylisoxazol-5- yl)methoxy)-1H-indol- 2-yl)methyl)azetidine-2- carboxamide | 374.11 | 375.2 |

### Example 68 (Compound 617)

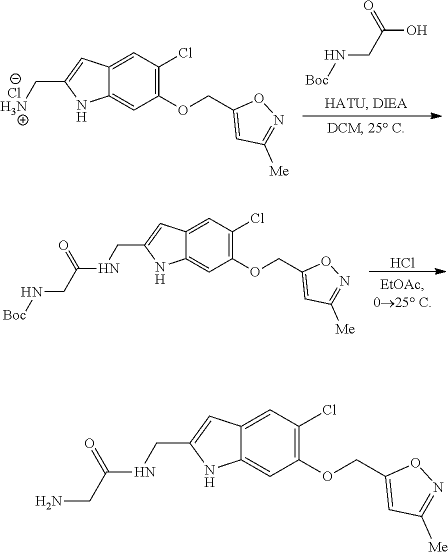 [SMILES: [Cl-] ; Cc1cc(COc2cc3[nH]c(C[NH3+])cc3cc2Cl)on1 ; CC(C)(C)OC(=O)NCC(=O)O ; Cc1cc(COc2cc3[nH]c(CNC(=O)CNC(=O)OC(C)(C)C)cc3cc2Cl)on1 ; Cl ; Cc1cc(COc2cc3[nH]c(CNC(=O)CN)cc3cc2Cl)on1]

### Step 1: tert-butyl (2-(((5-chloro-6-((3-methylisoxazol-5-yl)methoxy)-1H-indol-2-yl)methyl)amino)-2-oxoethyl)carbamate

To a solution of (5-chloro-6-((3-methylisoxazol-5-yl)methoxy)-1H-indol-2-yl)methanamine hydrochloride (200 mg, 609 μmol, 1.00 eq) in DCM (1 mL) was added 2-(tert-butoxycarbonylamino)acetic acid (138 mg, 792 μmol, 1.30 eq), HATU (301 mg, 792 μmol, 1.30 eq), and DIEA (393 mg, 3.05 mmol, 5.00 eq). The reaction mixture was stirred at 25° C. for 1 h. The reaction mixture was quenched by addition of H₂O (3 mL) and extracted with DCM (3×5 mL). The combined organic phases was washed with brine (3 mL), dried over Na₂SO₄, filtered, and concentrated under reduced pressure. Purification by column chromatography gave tert-butyl (2-(((5-chloro-6-((3-methylisoxazol-5-yl)methoxy)-1H-indol-2-yl)methyl)amino)-2-oxoethyl)carbamate. LC-MS (ESI) m/z calcd for C₂₁H₂₅ClN₄O₅: 448.15; found: 449.2 [M+H]⁺.

### Step 2: 2-amino-N-((5-chloro-6-((3-methylisoxazol-5-yl)methoxy)-1H-indol-2-yl)methyl)acetamide

To a solution of tert-butyl (2-(((5-chloro-6-((3-methylisoxazol-5-yl)methoxy)-1H-indol-2-yl)methyl)amino)-2-oxoethyl)carbamate (150 mg, 334 μmol, 1.00 eq) in EtOAc (2 mL) cooled to 0° C. was added HCl (4.00 M in EtOAc, 6.00 mL, 71.8 eq) dropwise. The reaction mixture was allowed to warm to 25° C. stirred for 0.5 h. The pH of the reaction mixture was adjusted to pH=7 by addition of saturated aqueous NaHCO₃, and the aqueous phase was extracted with ethyl acetate (3×5 mL). The combined organic phases was washed with brine (5 mL), dried over anhydrous Na₂SO₄, filtered, and concentrated under reduced pressure. Purification by prep-HPLC gave 2-amino-N-((5-chloro-6-((3-methylisoxazol-5-yl)methoxy)-1H-indol-2-yl)methyl)acetamide (Compound 617). LC-MS (ESI) m/z calcd for C₁₆H₁₇ClN₄O₃: 348.10; found: 349.1 [M+H]⁺. ¹H NMR (400 MHz, DMSO-d₆) δ 10.95 (s, 1H), 8.28 (s, 1H), 7.50 (s, 1H), 7.14 (s, 1H), 6.45 (s, 1H), 6.17 (s, 1H), 5.27 (s, 2H), 4.38 (d, J=4.8 Hz, 2H), 3.13 (s, 2H), 2.24 (s, 3H). ¹H NMR (400 MHz, MeOD-d₄) δ 7.46 (s, 1H), 7.06 (s, 1H), 6.35 (s, 1H), 6.24 (s, 1H), 5.20 (s, 2H), 4.50 (s, 2H), 3.32 (s, 2H), 2.28 (s, 3H).

### Example 69 (Compound 618)

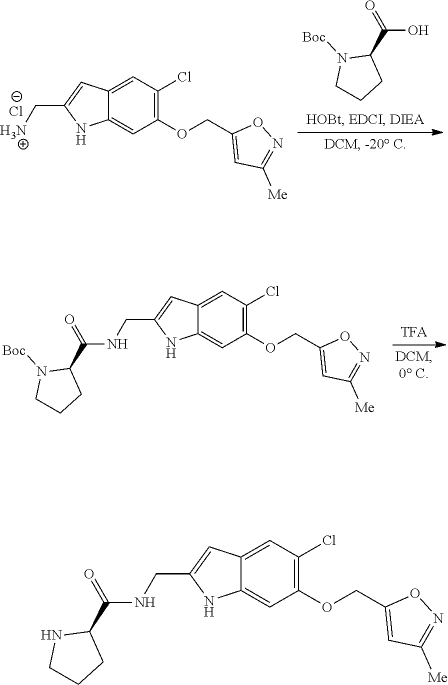 [SMILES: [Cl-] ; Cc1cc(COc2cc3[nH]c(C[NH3+])cc3cc2Cl)on1 ; CC(C)(C)OC(=O)N1CCC[C@@H]1C(=O)O ; Cc1cc(COc2cc3[nH]c(CNC(=O)[C@H]4CCCN4C(=O)OC(C)(C)C)cc3cc2Cl)on1 ; Cc1cc(COc2cc3[nH]c(CNC(=O)[C@H]4CCCN4)cc3cc2Cl)on1]

### Step 1: tert-butyl (R)-2-(((5-chloro-6-((3-methylisoxazol-5-yl)methoxy)-1H-indol-2-yl)methyl)carbamoyl)pyrrolidine-1-carboxylate

To a solution of (tert-butoxycarbonyl)-D-proline (144 mg, 670 μmol, 1.10 eq) in DCM (2 mL) cooled to −20° C. was added 1-(3-Dimethylaminopropyl)-3-ethylcarbodiimide hydrochloride (EDCI, 128 mg, 670 μmol, 1.10 eq) and 1H-benzo[d][1,2,3]triazol-1-ol (HOBt, 90.5 mg, 670 μmol, 1.10 eq). The mixture was stirred for 0.5 h. Then, a solution of (5-chloro-6-((3-methylisoxazol-5-yl)methoxy)-1H-indol-2-yl)methanamine hydrochloride (200 mg, 609 μmol, 1.00 eq) and DIEA (157 mg, 1.22 mmol, 2.00 eq) in DCM (1.00 mL) was added. The combined reaction mixture was stirred at −20° C. for 1 h and was then poured into water (5 mL) and stirred for 2 minutes. The aqueous phase was extracted with ethyl acetate (2×5 mL). The combined organic phase was washed with brine (2×5 mL), dried over anhydrous Na₂SO₄, filtered, and concentrated under reduced pressure. Purification by prep-TLC gave tert-butyl (R)-2-(((5-chloro-6-((3-methylisoxazol-5-yl)methoxy)-1H-indol-2-yl)methyl)carbamoyl)pyrrolidine-1-carboxylate. LC-MS (ESI) m/z calcd for C₂₄H₂₉ClN₄O₅: 488.18; found: 489.4 [M+H]⁺.

### Step 2: (R)—N-((5-chloro-6-((3-methylisoxazol-5-yl)methoxy)-1H-indol-2-yl)methyl)pyrrolidine-2-carboxamide

To a solution of tert-butyl (R)-2-(((5-chloro-6-((3-methylisoxazol-5-yl)methoxy)-1H-indol-2-yl)methyl)carbamoyl)pyrrolidine-1-carboxylate (210 mg, 429 μmol, 1.00 eq) in DCM (4 mL) was added TFA (614 mg, 5.38 mmol, 12.5 eq) dropwise at 0° C. The reaction mixture was stirred at 0° C. for 4 h. The reaction mixture was concentrated under reduced pressure. Purification by prep-HPLC gave (R)—N-((5-chloro-6-((3-methylisoxazol-5-yl)methoxy)-1H-indol-2-yl)methyl)pyrrolidine-2-carboxamide (Compound 618). LC-MS (ESI) m/z calcd for C₁₉H₂₁ClN₄O₃: 388.13; found: 389.2 [M+H]⁺. ¹H NMR (400 MHz, DMSO-d₆) δ 9.26 (s, 1H), 8.27 (t, J=5.6 Hz, 1H), 7.53 (s, 1H), 6.93 (s, 1H), 6.24 (s, 1H), 6.20 (s, 1H), 5.18 (s, 2H), 4.49-4.46 (m, 1H), 4.39-4.38 (m, 1H), 3.82-3.78 (m, 1H), 3.03-3.00 (m, 1H), 2.88-2.85 (m, 1H), 2.31 (s, 3H), 1.94-1.94 (m, 1H), 1.72-1.71 (m, 1H), 1.69-1.69 (m, 2H).

### Example 70 (Compound 619)

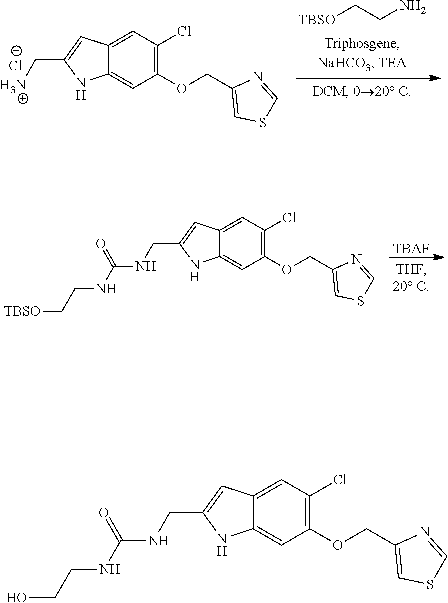

### Step 1: 1-(2-((tert-butyldimethylsilyl)oxy)ethyl)-3-((5-chloro-6-(thiazol-4-ylmethoxy)-1H-indol-2-yl)methyl)urea

To a solution of (5-chloro-6-(thiazol-4-ylmethoxy)-1H-indol-2-yl)methanamine hydrochloride (300 mg, 908 μmol, 1.00 eq) in DCM (6 mL) was added NaHCO₃ (153 mg, 1.82 mmol, 2.00 eq) in one portion. The reaction mixture was stirred for 30 min. Then the reaction mixture was degassed and purged with N₂ three times and cooled to 0° C. Bis(trichloromethyl) carbonate (0.134 g, 454 μmol, 0.500 eq) was added to the reaction mixture and stirred for 30 min. Then TEA (276 mg, 2.73 mmol, 3.00 eq) and 2-[tert-butyl(dimethyl)silyl]oxoethanamine (175 mg, 999 μmol, 1.10 eq) were added dropwise to the reaction mixture at 0° C. After the addition, the reaction mixture was warmed to 20° C. and stirred for 1 h. The reaction mixture was quenched by addition of saturated aqueous NaHCO₃ (30 mL) solution at 20° C. and extracted with DCM (3×10 mL). The combined organic phases were washed with brine (10 mL), dried with anhydrous Na₂SO₄, filtered, and concentrated under reduced pressure to give a residue. The residue was purified by column chromatography to give 1-(2-((tert-butyldimethylsilyl)oxy)ethyl)-3-((5-chloro-6-(thiazol-4-ylmethoxy)-1H-indol-2-yl)methyl)urea. LC-MS (ESI) m/z calcd for C₂₂H₃₁ClN₄O₃SSi: 494.16; found 495.2 [M+H]⁺.

### Step 2: 1-((5-chloro-6-(thiazol-4-ylmethoxy)-1H-indol-2-yl)methyl)-3-(2-hydroxyethyl)urea

To a solution of 1-(2-((tert-butyldimethylsilyl)oxy)ethyl)-3-((5-chloro-6-(thiazol-4-ylmethoxy)-1H-indol-2-yl)methyl)urea (120 mg, 242 μmol, 1.00 eq) in THF (2 mL) was added TBAF (1 M in THF, 1.2 mL, 5.00 eq) in one portion at 20° C. The reaction mixture was stirred at 20° C. for 1 h. The reaction mixture was quenched by addition of saturated aqueous NH₄Cl (30 mL) solution at 20° C. and extracted with dicholormethane:isopropyl alcohol (3:1, 3×10 mL). The combined organic phases was washed with brine (10 mL), dried over anhydrous Na₂SO₄, filtered, and concentrated under reduced pressure. The residue was purified by prep-HPLC to give 1-((5-chloro-6-(thiazol-4-ylmethoxy)-1H-indol-2-yl)methyl)-3-(2-hydroxyethyl)urea (Compound 619). LC-MS (ESI) m/z calcd for C₁₆H₁₇ClN₄O₃S: 380.07; found 381.1 [M+H]⁺. ¹H NMR (400 MHz, DMSO-d₆) δ 10.86 (s, 1H), 9.14 (d, J=2.0 Hz, 1H), 7.75 (d, J=2.0 Hz, 1H), 7.49 (s, 1H), 7.17 (s, 1H), 6.37 (t, J=5.6 Hz, 1H), 6.13 (s, 1H), 6.02 (t, J=5.2 Hz, 1H), 5.26 (s, 2H), 4.66 (t, J=5.2 Hz, 1H), 4.27 (d, J=5.6 Hz, 2H), 3.41-3.36 (m, 2H), 3.11-3.07 (m, 2H).

### Example 71 (Compound 620)

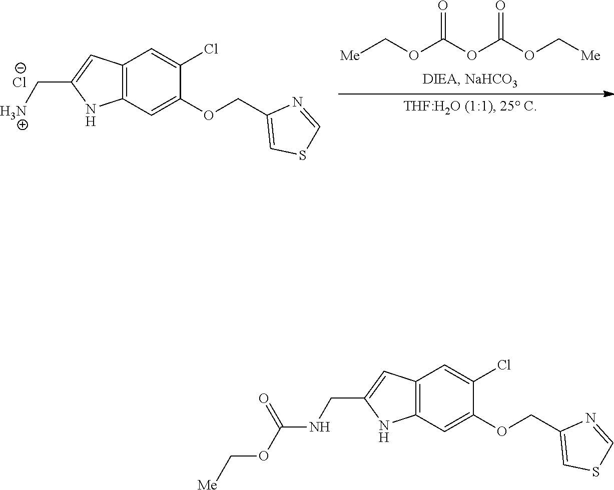 [SMILES: [Cl-] ; [NH3+]Cc1cc2cc(Cl)c(OCc3cscn3)cc2[nH]1 ; CCOC(=O)OC(=O)OCC ; CCOC(=O)NCc1cc2cc(Cl)c(OCc3cscn3)cc2[nH]1]

### Step 1: ethyl ((5-chloro-6-(thiazol-4-ylmethoxy)-1H-indol-2-yl)methyl)carbamate

To a mixture of (5-chloro-6-(thiazol-4-ylmethoxy)-1H-indol-2-yl)methanamine hydrochloride (150 mg, 454 μmol, 1.00 eq, HCl salt) and diethyl dicarbonate (67.0 mg, 499 μmol, 1.10 eq) in H₂O (1 mL) and THF (1 mL) was added DIEA (11.0 mg, 908 μmol, 2.00 eq) and NaHCO₃ (76.3 mg, 908 μmol, 2.00 eq). The reaction mixture was stirred at 25° C. for 1 h. The reaction mixture was quenched by addition of H₂O (100 mL) and extracted with ethyl acetate (3×50 mL). The combined organic phases were washed with brine (50 mL), dried over anhydrous Na₂SO₄, filtered, and concentrated under reduced pressure. Purification by prep-HPLC gave ethyl ((5-chloro-6-(thiazol-4-ylmethoxy)-1H-indol-2-yl)methyl)carbamate (Compound 620). LC-MS (ESI) m/z calcd for C₁₆H₁₆ClN₃O₃S: 365.06; found: 366.1[M+H]⁺. ¹H NMR (400 MHz, DMSO-d₆) δ 10.89 (s, 1H), 9.14 (s, 1H), 7.75 (d, J=1.6 Hz, 1H), 7.57 (t, J=6.0 Hz, 1H), 7.50 (s, 1H), 7.16 (s, 1H), 6.14 (s, 1H), 5.27 (s, 2H), 4.27 (d, J=5.6 Hz, 2H), 4.05-3.99 (m, 2H), 1.17 (t, J=7.2 Hz, 3H).

The following compounds in Table T-40 were synthesized using procedures similar to Compound 620 using the appropriate starting materials.

TABLE T-40

| Com- | | | Exact | LCMS, |
|---|---|---|---|---|
| pound | | | mass | found |
| No. | Structure | IUPAC name | (g/mol) | [M + H]⁺ |
| 621 | 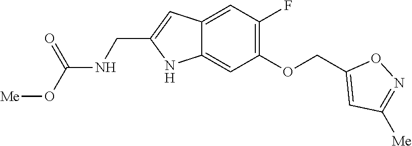 [SMILES: COC(=O)NCc1cc2cc(F)c(OCc3cc(C)no3)cc2[nH]1] | methyl ((5-fluoro-6-((3- methylisoxazol-5- yl)methoxy)-1H-indol- 2-yl)methyl)carbamate | 333.11 | 334.1 |
| 622 | 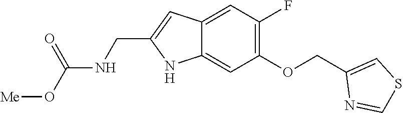 [SMILES: COC(=O)NCc1cc2cc(F)c(OCc3cscn3)cc2[nH]1] | methyl ((5-fluoro-6- (thiazol-4-ylmethoxy)- 1H-indol-2- yl)methyl)carbamate | 335.07 | 336.1 |

### Example 72 (Compound 623)

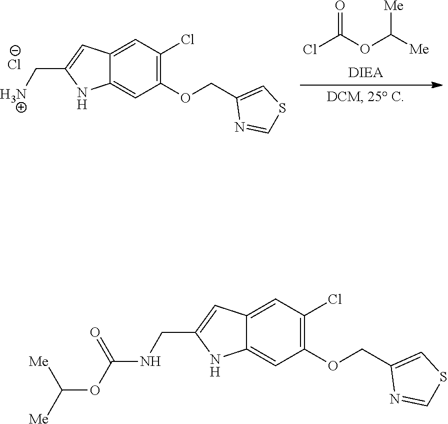 [SMILES: [Cl-] ; [NH3+]Cc1cc2cc(Cl)c(OCc3cscn3)cc2[nH]1 ; CC(C)OC(=O)Cl ; CC(C)OC(=O)NCc1cc2cc(Cl)c(OCc3cscn3)cc2[nH]1]

### Step 1: isopropyl ((5-chloro-6-(thiazol-4-ylmethoxy)-1H-indol-2-yl)methyl)carbamate

To a solution of (5-chloro-6-(thiazol-4-ylmethoxy)-1H-indol-2-yl)methanamine hydrochloride (150 mg, 454 μmol, 1.00 eq) in DCM (3 mL) was added DIEA (176 mg, 1.36 mmol, 3.00 eq) and isopropyl chloroformate (61.2 mg, 499 μmol, 1.10 eq) in one portion. The reaction mixture was stirred at 25° C. for 1 h. The reaction mixture was quenched by addition of water (8 mL) and extracted with EtOAc (3×10 mL). The combined organic phases were dried over Na₂SO₄, filtered, and concentrated under reduced pressure. Purification by prep-HPLC gave isopropyl ((5-chloro-6-(thiazol-4-ylmethoxy)-1H-indol-2-yl)methyl)carbamate (Compound 623). LC-MS (ESI) m/z calcd for C₁₇H₁₈ClN₃O₃S: 379.08; found: 380.1 [M+H]⁺. ¹H NMR (400 MHz, DMSO-d₆) δ 10.8 (s, 1H), 9.14 (s, 1H), 7.75 (s, 1H), 7.49 (s, 2H), 7.20 (s, 1H), 6.13 (s, 1H), 5.27 (s, 2H), 4.83-4.74 (m, 1H), 4.25 (t, J=5.6 Hz, 2H), 1.18 (d, J=6.0 Hz, 6H).

The following compounds in Table T-41 were synthesized using procedures similar to Compound 623 using the appropriate starting materials.

TABLE T-41

| | | | Exact | LCMS, |
|---|---|---|---|---|
| Compound | | | mass | found |
| No. | Structure | IUPAC name | (g/mol) | [M + H]⁺ |
| 624 | 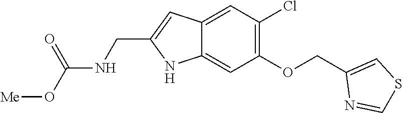 [SMILES: COC(=O)NCc1cc2cc(Cl)c(OCc3cscn3)cc2[nH]1] | methyl ((5-chloro-6- (thiazol-4-ylmethoxy)- 1H-indol-2- yl)methyl)carbamate | 351.04 | 351.8 |

### Example 73 (Compound 625)

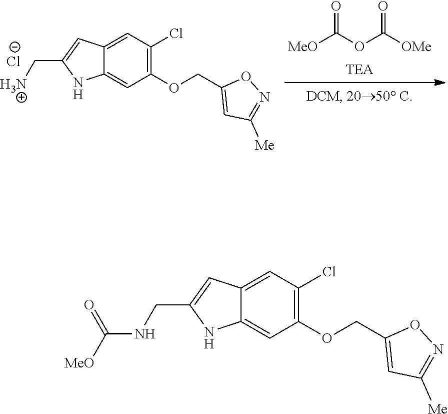 [SMILES: [Cl-] ; Cc1cc(COc2cc3[nH]c(C[NH3+])cc3cc2Cl)on1 ; COC(=O)OC(=O)OC ; COC(=O)NCc1cc2cc(Cl)c(OCc3cc(C)no3)cc2[nH]1]

### Step 1: methyl ((5-chloro-6-((3-methylisoxazol-5-yl)methoxy)-1H-indol-2-yl)methyl)carbamate

To a mixture of (5-chloro-6-((3-methylisoxazol-5-yl)methoxy)-1H-indol-2-yl)methanamine hydrochloride (90.0 mg, 274 μmol, 1.00 eq) and dimethyl dicarbonate (40.4 mg, 301 μmol, 1.10 eq) in DCM (2 mL) was added TEA (138 mg, 1.37 mmol, 5.00 eq) in one portion at 20° C. The reaction mixture was heated to 50° C. and stirred for 4 h. The reaction mixture was quenched by addition of H₂O (3 mL) and then extracted with ethyl acetate (10 mL×3). The combined organic phases were washed with brine (20 mL), dried over anhydrous Na₂SO₄, filtered, and concentrated under reduced pressure to give a residue. The residue was purified by prep-HPLC to give methyl ((5-chloro-6-((3-methylisoxazol-5-yl)methoxy)-1H-indol-2-yl)methyl)carbamate (Compound 625). LC-MS (ESI) m/z calcd for C₁₆H₁₆ClN₃O₄: 349.08; found 350.1 [M+H]⁺. ¹H NMR (400 MHz, DMSO-d₆) δ 10.9 (s, 1H), 7.63 (t, J=5.2 Hz, 1H), 7.51 (s, 1H), 7.13 (s, 1H), 6.45 (s, 1H), 6.15 (s, 1H), 5.27 (s, 2H), 4.28 (d, J=5.6 Hz, 2H), 3.57 (s, 3H), 2.24 (s, 3H).

### Example 74 (Compound 626)

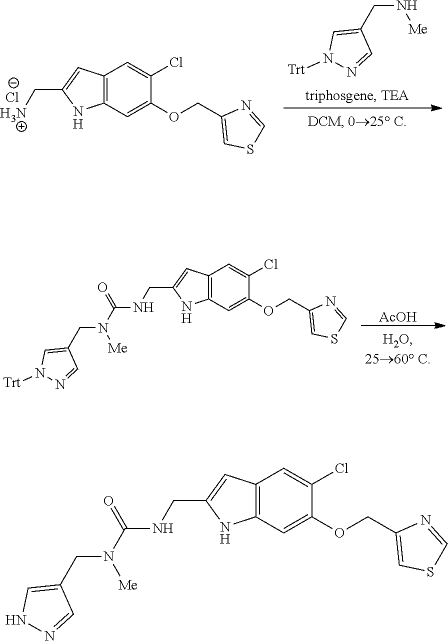

### Step 1: 3-((5-chloro-6-(thiazol-4-ylmethoxy)-1H-indol-2-yl)methyl)-1-methyl-1-((1-trityl-1H-pyrazol-4-yl)methyl)urea

To a solution of (5-chloro-6-(thiazol-4-ylmethoxy)-1H-indol-2-yl)methanamine (170 mg, 515 μmol, 1.00 eq, HCl salt) and N-methyl-1-(1-trityl-1H-pyrazol-4-yl)methanamine (218 mg, 618 μmol, 1.20 eq) in DCM (1 mL) was added TEA (208 mg, 2.06 mmol, 4.00 eq) and triphosgene (76.4 mg, 257 μmol, 0.500 eq) in portions at 0° C. under N₂ atmosphere. The reaction mixture was allowed to warm to 25° C. and stirred for 2 h. The reaction mixture was quenched by addition H₂O (20 mL) at 25° C. and extracted with EtOAc (3×15 mL). The combined organic phases were washed with brine (2×15 mL), dried over Na₂SO₄, filtered, and concentrated under reduced pressure. Purification by column chromatography gave 3-((5-chloro-6-(thiazol-4-ylmethoxy)-1H-indol-2-yl)methyl)-1-methyl-1-((1-trityl-1H-pyrazol-4-yl)methyl)urea.

### Step 2: 1-((1H-pyrazol-4-yl)methyl)-3-((5-chloro-6-(thiazol-4-ylmethoxy)-1H-indol-2-yl)methyl)-1-methylurea

To a solution of 3-((5-chloro-6-(thiazol-4-ylmethoxy)-1H-indol-2-yl)methyl)-1-methyl-1-((1-trityl-1H-pyrazol-4-yl)methyl)urea (60.0 mg, 89.1 μmol, 1.00 eq) in H₂O (0.1 mL) was added acetic acid (126 mg, 2.10 mmol, 23.5 eq) at 25° C. under N₂ atmosphere. The reaction mixture was heated to 60° C. and stirred for 2 h. After cooling to room temperature, the reaction mixture was filtered. The filtrate was diluted with H₂O (10 mL) and extracted with EtOAc (3×5 mL). The combined organic layers were washed with brine (3×5 mL), dried over Na₂SO₄, filtered, and concentrated under reduced pressure. Purification by prep-HPLC gave 1-((1H-pyrazol-4-yl)methyl)-3-((5-chloro-6-(thiazol-4-ylmethoxy)-1H-indol-2-yl)methyl)-1-methylurea (Compound 626). LC-MS (ESI) m/z calcd for C₁₉H₁₉ClN₆O₂S: 430.10; found 431.2 [M+H]⁺. ¹H NMR (400 MHz, DMSO-d₆) δ 12.65 (s, 1H), 10.75 (s, 1H), 9.15 (s, 1H), 7.76 (d, J=1.6 Hz, 1H), 7.58-7.48 (m, 1H), 7.43-7.42 (m, 2H), 7.21 (s, 1H), 6.86 (t, J=5.2 Hz, 1H), 6.11 (s, 1H), 5.26 (s, 2H), 4.32 (d, J=5.6 Hz, 2H), 4.28 (s, 2H), 2.76 (s, 3H).

The following compounds in Table T-42 were synthesized using procedures similar to Compound 626 using the appropriate starting materials.

TABLE T-42

| | | | Exact | LCMS, |
|---|---|---|---|---|
| Compound | | | mass | found |
| No. | Structure | IUPAC name | (g/mol) | [M + H]⁺ |
| 627 | 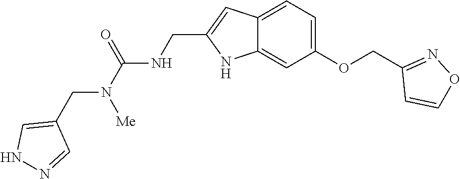 [SMILES: CN(Cc1cn[nH]c1)C(=O)NCc1cc2ccc(OCc3ccon3)cc2[nH]1] | 1-((1H-pyrazol-4- yl)methyl)-3-((6- (isoxazol-3-ylmethoxy)- 1H-indol-2-yl)methyl)- 1-methylurea | 380.16 | 381.2 |
| 628 | 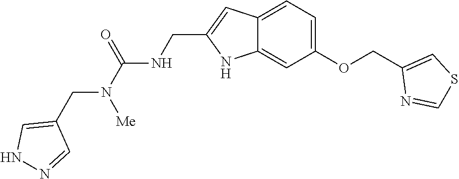 [SMILES: CN(Cc1cn[nH]c1)C(=O)NCc1cc2ccc(OCc3cscn3)cc2[nH]1] | 1-((1H-pyrazol-4- yl)methyl)-1-methyl-3- ((6-(thiazol-4- ylmethoxy)-1H-indol-2- yl)methyl)urea | 396.14 | 397.3 |
| 629 | 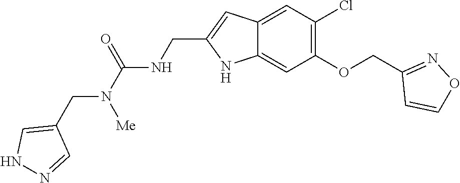 [SMILES: CN(Cc1cn[nH]c1)C(=O)NCc1cc2cc(Cl)c(OCc3ccon3)cc2[nH]1] | 1-((1H-pyrazol-4- yl)methyl)-3-((5-chloro- 6-(isoxazol-3- ylmethoxy)-1H-indol-2- yl)methyl)-1-methylurea | 414.12 | 415.2 |

### Example 75 (Compound 630)

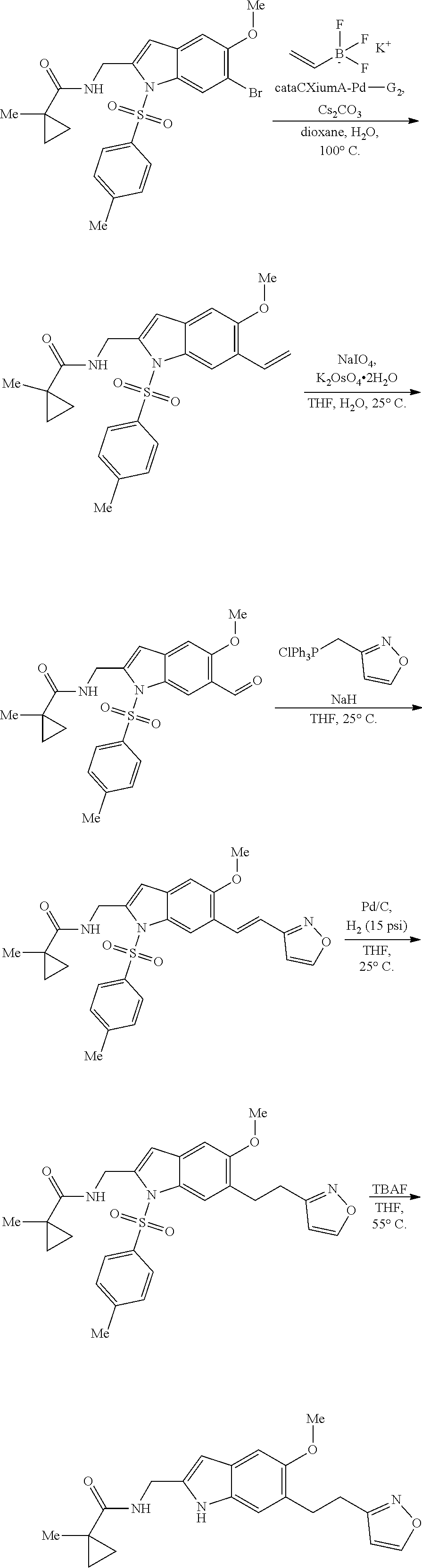

### Step 1: N-((5-methoxy-1-tosyl-6-vinyl-1H-indol-2-yl)methyl)-1-methylcyclopropane-1-carboxamide

To a mixture of N-((6-bromo-5-methoxy-1-tosyl-1H-indol-2-yl)methyl)-1-methylcyclopropane-1-carboxamide (3.00 g, 6.11 mmol, 1.00 eq) and potassium vinyltrifluoroborate (981 mg, 7.33 mmol, 1.20 eq) in dioxane (30 mL) and H₂O (6 mL) was added Cs₂CO₃ (3.98 g, 12.2 mmol, 2.00 eq) in one portion at 20° C. under N₂. The reaction mixture was degassed and purged with N₂ three times, and then chloro[(di(1-adamantyl)-N-butylphosphine)-2-(2-aminobiphenyl)]palladium (cataCXiumA-Pd-G2, 408 mg, 611 μmol, 0.10 eq) was added. The reaction mixture was warmed to 100° C. and stirred for 2 h. The reaction mixture was allowed to cool to room temperature and was quenched by addition of water (30 mL) and extracted with EtOAc (3×30 mL). The combined organic phase was washed with H₂O (3×30 mL), dried over Na₂SO₄, filtered, and concentrated under reduced pressure. Purification by column chromatography gave N-((5-methoxy-1-tosyl-6-vinyl-1H-indol-2-yl)methyl)-1-methylcyclopropane-1-carboxamide. LC-MS (ESI) m/z calcd for C₂₄H₂₆N₂O₄S: 438.16; found: 439.2 [M+H]⁺.

### Step 2: N-((6-formyl-5-methoxy-1-tosyl-1H-indol-2-yl)methyl)-1-methylcyclopropane-1-carboxamide

To a solution of N-((5-methoxy-1-tosyl-6-vinyl-1H-indol-2-yl)methyl)-1-methylcyclopropane-1-carboxamide (2.30 g, 5.24 mmol, 1.00 eq) in THF (23 mL) and H₂O (2.3 mL) was added NaIO₄ (4.49 g, 21.0 mmol, 4.00 eq) and potassium osmate dihydrate (96.6 mg, 262 μmol, 0.05 eq). The reaction mixture was stirred at 25° C. for 2 h. The reaction mixture was quenched by addition of saturated aqueous Na₂S₂O₃ solution (50 mL) and extracted with EtOAc (3×25 mL). The combined organic phases were washed with water (3×25 mL), dried over Na₂SO₄, filtered, and concentrated under reduced pressure. Purification by column chromatography gave N-((6-formyl-5-methoxy-1-tosyl-1H-indol-2-yl)methyl)-1-methylcyclopropane-1-carboxamide. LC-MS (ESI) m/z calcd for C₂₃H₂₄N₂O₅S: 440.14; found: 441.2 [M+H]⁺.

### Step 3: (E)-N-((6-(2-(isoxazol-3-yl)vinyl)-5-methoxy-1-tosyl-1H-indol-2-yl)methyl)-1-methylcyclopropane-1-carboxamide

To a solution of 3-((chlorotriphenyl-λ⁵-phosphanyl)methyl)isoxazole (431 mg, 1.14 mmol, 1.00 eq) in THF (5 mL) cooled to 0° C. was added NaH (59.0 mg, 1.48 mmol, 60.0% w/w, 1.30 eq) in portions under N₂. The reaction mixture was stirred at 0° C. for 15 mins, then N-((6-formyl-5-methoxy-1-tosyl-1H-indol-2-yl)methyl)-1-methylcyclopropane-1-carboxamide (500 mg, 1.14 mmol, 1.00 eq) was added. The reaction mixture was allowed to warm to room temperature and stirred for 3 h. The reaction mixture was quenched by addition of water (30 mL) and extracted with EtOAc (3×15 mL). The combined organic phases were washed with water (3×15 mL), dried over Na₂SO₄, filtered, and concentrated under reduced pressure. Purification by column chromatography gave (E)-N-((6-(2-(isoxazol-3-yl)vinyl)-5-methoxy-1-tosyl-1H-indol-2-yl)methyl)-1-methylcyclopropane-1-carboxamide. LC-MS (ESI) m/z calcd for C₂₇H₂₇N₃O₅S: 505.17; found: 506.3 [M+H]⁺.

### Step 4: N-((6-(2-(isoxazol-3-yl)ethyl)-5-methoxy-1-tosyl-1H-indol-2-yl)methyl)-1-methylcyclopropane-1-carboxamide

To a solution of (E)-N-((6-(2-(isoxazol-3-yl)vinyl)-5-methoxy-1-tosyl-1H-indol-2-yl)methyl)-1-methylcyclopropane-1-carboxamide (380 mg, 752 μmol, 1.00 eq) in THF (3.0 mL) was added Pd/C (412 mg, 1.13 mmol, 10.0% purity, 1.50 eq) under N₂. The reaction mixture was degassed under vacuum and purged with H₂ three times. The reaction mixture was stirred at 25° C. under an atmosphere of H₂ (15 psi) for 20 min. The reaction mixture was filtered through a pad of Celite. Concentration of the filtrate gave N-((6-(2-(isoxazol-3-yl)ethyl)-5-methoxy-1-tosyl-1H-indol-2-yl)methyl)-1-methylcyclopropane-1-carboxamide, which was used without further purification. LC-MS (ESI) m/z calcd for C₂₇H₂₉N₃O₅S: 507.18; found: 508.3 [M+H]⁺.

### Step 5: N-((6-(2-(isoxazol-3-yl)ethyl)-5-methoxy-1H-indol-2-yl)methyl)-1-methylcyclopropane-1-carboxamide

To a solution of N-((6-(2-(isoxazol-3-yl)ethyl)-5-methoxy-1-tosyl-1H-indol-2-yl)methyl)-1-methylcyclopropane-1-carboxamide (180 mg, 355 μmol, 1.00 eq) in THF (1.0 mL) was added TBAF (1 M in THF, 1.06 mL, 3.00 eq) under N₂. The reaction mixture was warmed to 55° C. and stirred for 12 h. The reaction mixture was allowed to cool to room temperature and quenched by addition of water (20 mL) and extracted with EtOAc (3×10 mL). The combined organic phase was washed with water (3×10 mL), dried over Na₂SO₄, filtered, and concentrated under reduced pressure. Purification by prep-HPLC gave N-((6-(2-(isoxazol-3-yl)ethyl)-5-methoxy-1H-indol-2-yl)methyl)-1-methylcyclopropane-1-carboxamide (Compound 630). LC-MS (ESI) m/z calcd for C₂₀H₂₃N₃O₃: 353.17; found 354.3 [M+H]⁺. ¹H NMR (400 MHz, DMSO-d₆) δ 10.4 (s, 1H), 8.75 (s, 1H), 7.96 (t, J=5.6 Hz, 1H), 7.07 (s, 1H), 6.95 (s, 1H), 6.45 (d, J=1.60 Hz, 1H), 6.09 (s, 1H), 4.34 (d, J=5.60 Hz, 2H), 3.77 (s, 3H), 2.94-2.90 (m, 4H), 1.28 (s, 3H), 1.00-0.97 (m, 2H), 0.53-0.51 (m, 2H).

The following compounds in Table T-43 were synthesized using procedures similar to Compound 630 using the appropriate starting materials.

TABLE T-43

| | | | Exact | LCMS, |
|---|---|---|---|---|
| Compound | | | mass | found |
| No. | Structure | IUPAC name | (g/mol) | [M + H]⁺ |
| 631 | 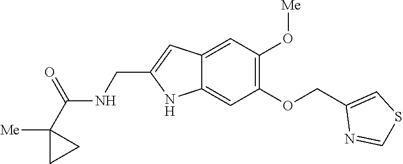 [SMILES: COc1cc2cc(CNC(=O)C3(C)CC3)[nH]c2cc1OCc1cscn1] | N-((5-methoxy-6-(2- (thiazol-4-yl)ethyl)-1H- indol-2-yl)methyl)-1- methylcyclopropane-1- carboxamide | 369.15 | 370.3 |

### Example 76 (Compound 632)

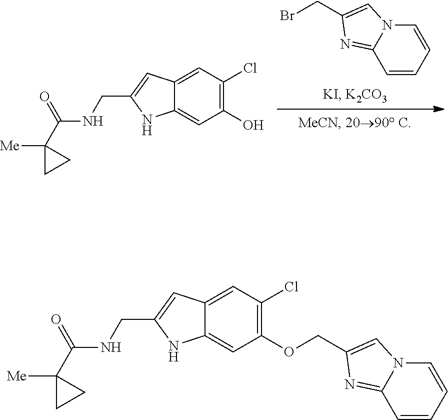 [SMILES: CC1(C(=O)NCc2cc3cc(Cl)c(O)cc3[nH]2)CC1 ; BrCc1cn2ccccc2n1 ; CC1(C(=O)NCc2cc3cc(Cl)c(OCc4cn5ccccc5n4)cc3[nH]2)CC1]

### Step 1: N-((5-chloro-6-(imidazo[1,2-a]pyridin-2-ylmethoxy)-1H-indol-2-yl)methyl)-1-methylcyclopropane-1-carboxamide

To a mixture of 2-(bromomethyl)imidazo[1,2-a]pyridine (60.0 mg, 284 μmol, 1.00 eq) and N-((5-chloro-6-hydroxy-1H-indol-2-yl)methyl)-1-methylcyclopropane-1-carboxamide (63.3 mg, 227 μmol, 0.80 eq) in MeCN (3 mL) was added KI (47.1 mg, 284 μmol, 1.00 eq) and K₂CO₃ (78.5 mg, 568 μmol, 2.00 eq) in one portion at 20° C. under N₂. The reaction mixture was heated and stirred at 90° C. for 4 h. After cooling to room temperature, the reaction mixture was quenched by addition of H₂O (10 mL) at 20° C. and extracted with ethyl acetate (10 mL×3). The combined organic layers were dried over Na₂SO₄, filtered, and concentrated under reduced pressure to give a residue. The residue was purified by prep-HPLC to give N-((5-chloro-6-(imidazo[1,2-a]pyridin-2-ylmethoxy)-1H-indol-2-yl)methyl)-1-methylcyclopropane-1-carboxamide (Compound 632). LC-MS (ESI) m/z calcd for C₂₂H₂₁ClN₄O₂: 408.14; found: 409.2 [M+H]⁺. ¹H NMR (400 MHz, DMSO-d₆) δ 10.75 (s, 1H), 8.55 (d, J=6.8 Hz, 1H), 8.00-7.98 (m, 2H), 7.54 (d, J=9.6 Hz, 1H), 7.49 (s, 1H), 7.27-7.22 (m, 2H), 6.91 (t, J=6.8 Hz, 1H), 6.11 (s, 1H), 5.26 (s, 2H), 4.34 (d, J=5.6 Hz, 2H), 1.28 (s, 3H), 1.00-0.98 (m, 2H), 0.54-0.51 (m, 2H).

The following compounds in Table T-44 were synthesized using procedures similar to Compound 632 using the appropriate starting materials.

TABLE T-44

| | | | Exact | LCMS, |
|---|---|---|---|---|
| Compound | | | mass | found |
| No. | Structure | IUPAC name | (g/mol) | [M + H]⁺ |
| 633 | 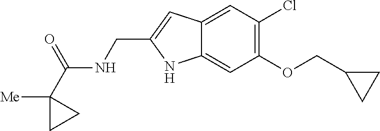 [SMILES: CC1(C(=O)NCc2cc3cc(Cl)c(OCC4CC4)cc3[nH]2)CC1] | N-((5-chloro-6- (cyclopropylmethoxy)- 1H-indol-2-yl)methyl)- 1-methylcyclopropane- 1-carboxamide | 332.13 | 333.2 |
| 634 | 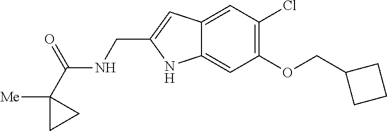 [SMILES: CC1(C(=O)NCc2cc3cc(Cl)c(OCC4CCC4)cc3[nH]2)CC1] | N-((5-chloro-6- (cyclobutylmethoxy)- 1H-indol-2-y1)methyl)- 1-methylcyclopropane- 1-carboxamide | 346.14 | 347.2 |
| 635 | 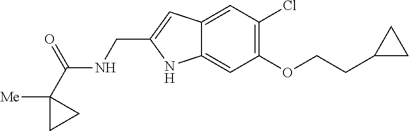 [SMILES: CC1(C(=O)NCc2cc3cc(Cl)c(OCCC4CC4)cc3[nH]2)CC1] | N-((5-chloro-6-(2- cyclopropylethoxy)-1H- indol-2-yl)methyl)-1- methylcyclopropane-1- carboxamide | 346.14 | 347.2 |

### Example 77 (Compound 636)

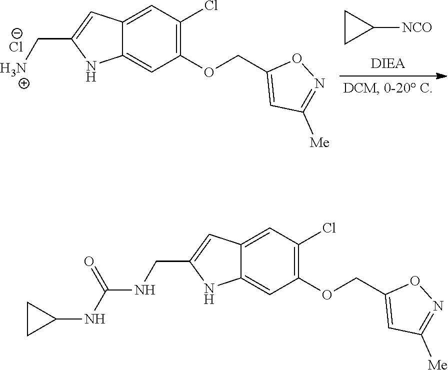

### Step 1: 1-((5-chloro-6-((3-methylisoxazol-5-yl)methoxy)-1H-indol-2-yl) methyl)-3-cyclopropylurea

To a solution of (5-chloro-6-((3-methylisoxazol-5-yl)methoxy)-1H-indol-2-yl)methanamine hydrochloride (100 mg, 305 μmol, 1.00 eq) in DCM (2 mL) at 0° C. was added DIEA (118 mg, 914 mol, 3.00 eq) and isocyanatocyclopropane (31.0 mg, 364 μmol, 1.20 eq) dropwise. The reaction was allowed to warm to 25° C. After 2 h, the reaction was quenched by addition of H₂O (15 mL). The solution was extracted with ethyl acetate (3×30 mL). The combined organic layers were washed with brine (20 mL), dried over anhydrous Na₂SO₄, filtered, and concentrated under reduced pressure. Purification by prep-HPLC gave 1-((5-chloro-6-((3-methylisoxazol-5-yl)methoxy)-1H-indol-2-yl) methyl)-3-cyclopropylurea (Compound 636). LC-MS (ESI) m/z calcd for C₁₈H₁₉ClN₄O₃: 374.11; found 375.0 [M+H]⁺. ¹H NMR (400 MHz, DMSO-d₆) δ ppm 10.89 (s, 1H), 7.50 (s, 1H), 7.16 (s, 1H), 6.46 (s, 1H), 6.35-6.27 (m, 2H), 6.13 (s, 1H), 5.27 (s, 2H), 4.28 (d, J=5.6 Hz, 2H), 2.45-2.41 (m, 1H), 2.24 (s, 3H), 0.59-0.55 (m, 2H), 0.37-0.33 (m, 2H).

The following compounds in Table T-45 were synthesized using procedures similar to Compound 636 using the appropriate starting materials.

TABLE T-45

| | | | Exact | LCMS, |
|---|---|---|---|---|
| Compound | | | mass | found |
| No. | Structure | IUPAC name | (g/mol) | [M + H]⁺ |
| 637 | 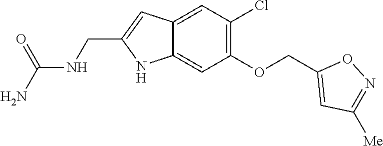 [SMILES: Cc1cc(COc2cc3[nH]c(CNC(N)=O)cc3cc2Cl)on1] | 1-((5-chloro-6-((3- methylisoxazol-5- yl)methoxy)-1H-indol- 2-yl)methyl)urea | 334.08 | 335.1 |
| 638 | 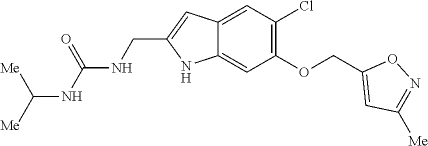 [SMILES: Cc1cc(COc2cc3[nH]c(CNC(=O)NC(C)C)cc3cc2Cl)on1] | 1-((5-chloro-6-((3- methylisoxazol-5- yl)methoxy)-1H-indol- 2-yl)methyl)-3- isopropylurea | 376.13 | 377.2 |

### Example 78 (Compound 639)

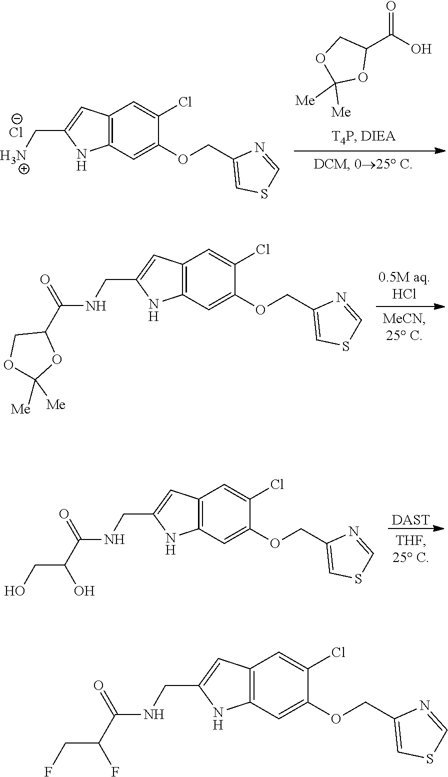

### Step 1: N-((5-chloro-6-(thiazol-4-ylmethoxy)-1H-indol-2-yl)methyl)-2,2-dimethyl-1,3-dioxolane-4-carboxamide

A solution of (5-chloro-6-(thiazol-4-ylmethoxy)-1H-indol-2-yl)methanamine hydrochloride (500 mg, 1.51 mmol, 1.00 eq, HCl salt) in DCM (5 mL) was cooled to 0° C., and then 2,2-dimethyl-1,3-dioxolane-4-carboxylic acid (265 mg, 1.82 mmol, 1.20 eq), DIEA (978 mg, 7.57 mmol, 1.32 mL, 5.00 eq) and 2,4,6-tributyl-1,3,5,2,4,6-trioxatriphosphinane 2,4,6-trioxide (T₄P, 1.31 g, 1.82 mmol, 1.20 eq) were added in portions at 0° C. After addition, the reaction mixture was warmed to 25° C. and stirred for 1 h. The reaction mixture was diluted with H₂O (10 mL) and extracted with DCM (3×5 mL). The combined organic phase was washed with brine (5 mL), dried over anhydrous Na₂SO₄, filtered, and concentrated under reduced pressure to give N-((5-chloro-6-(thiazol-4-ylmethoxy)-1H-indol-2-yl)methyl)-2,2-dimethyl-1,3-dioxolane-4-carboxamide, which was used in next step without of further purification.

### Step 2: N-((5-chloro-6-(thiazol-4-ylmethoxy)-1H-indol-2-yl)methyl)-2,3-dihydroxypropanamide

To a solution of N-((5-chloro-6-(thiazol-4-ylmethoxy)-1H-indol-2-yl)methyl)-2,2-dimethyl-1,3-dioxolane-4-carboxamide (500 mg, 1.19 mmol, 1.00 eq) in MeCN (5 mL) was added aqueous HCl (0.5 M in H2O, 1.19 mL, 0.50 eq) dropwise at 25° C. The resulting reaction mixture was stirred at 25° C. for 1 h. Purification by prep-HPLC gave N-((5-chloro-6-(thiazol-4-ylmethoxy)-1H-indol-2-yl)methyl)-2,3-dihydroxypropanamide. LC-MS (ESI) m/z calcd for C₁₆H₁₆ClN₃O₄S: 381.06; found: 382.0 [M+H]⁺.

### Step 3: N-((5-chloro-6-(thiazol-4-ylmethoxy)-1H-indol-2-yl)methyl)-2,3-difluoropropanamide

To a solution of N-((5-chloro-6-(thiazol-4-ylmethoxy)-1H-indol-2-yl)methyl)-2,3-dihydroxypropanamide (300 mg, 785 μmol, 1.00 eq) in THF (5 mL) was added DAST (379 mg, 2.36 mmol, 3.00 eq). The resulting reaction mixture was stirred at 25° C. for 3 h. The reaction mixture was diluted with H₂O (10 mL) and extracted with DCM (3×3 mL). The combined organic layers were washed with brine (2×3 mL), dried over Na₂SO₄, filtered, and concentrated under reduced pressure. Purification by prep-HPLC gave N-((5-chloro-6-(thiazol-4-ylmethoxy)-1H-indol-2-yl)methyl)-2,3-difluoropropanamide (Compound 639). LC-MS (ESI) m/z calcd for C₁₆H₁₄ClF₂N₃O₂S: 385.05; found: 386.0 [M+H]⁺. ¹H NMR (400 MHz, DMSO-d₆) δ 10.89 (s, 1H), 9.14 (d, J=1.6 Hz, 1H), 8.85 (t, J=5.6 Hz, 1H), 7.75 (s, 1H), 7.50 (s, 1H), 7.18 (s, 1H), 6.16 (s, 1H), 5.36-5.27 (m, 3H), 4.89-4.70 (m, 2H), 4.45-4.41 (m, 2H).

### Example 79 (Compound 640)

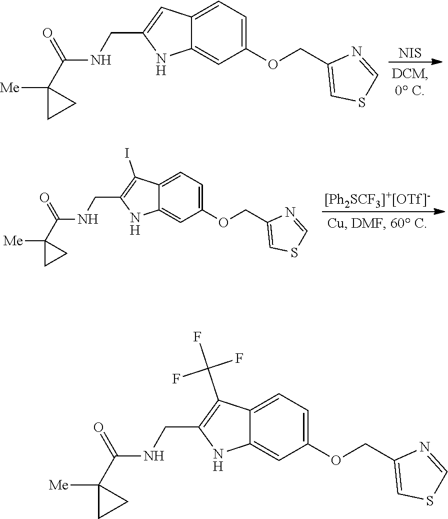

### Step 1: N-((3-iodo-6-(thiazol-4-ylmethoxy)-1H-indol-2-yl)methyl)-1-methylcyclopropane-1-carboxamide

A solution of 1-methyl-N-((6-(thiazol-4-ylmethoxy)-1H-indol-2-yl) methyl) cyclopropane-1-carboxamide (200 mg, 585 μmol, 1.00 eq) in DCM (2 mL) was cooled to 0° C., and 1-iodopyrrolidine-2,5-dione (NIS, 132 mg, 585 mol, 1.00 eq) was added in portions at 0° C. The reaction mixture was stirred at 0° C. for 0.5 h. The reaction mixture was poured into ice water (20 mL) and stirred for 5 mins. The aqueous phase was extracted with ethyl acetate (3×20 mL). The combined organic phases were washed with brine (20 mL), dried over anhydrous Na₂SO₄, filtered and concentrated under reduced pressure to give N-((3-iodo-6-(thiazol-4-ylmethoxy)-1H-indol-2-yl) methyl)-1-methylcyclopropane-1-carboxamide, which was used into the next step without further purification. LC-MS (ESI) m/z calcd for C₁₈H₁₈IN₃O₂S: 467.02; found 468.0 [M+H]⁺.

### Step 2: 1-methyl-N-((6-(thiazol-4-ylmethoxy)-3-(trifluoromethyl)-1H-indol-2-yl)methyl)cyclopropane-1-carboxamide

To a solution of N-((3-iodo-6-(thiazol-4-ylmethoxy)-1H-indol-2-yl)methyl)-1-methylcyclopropane-1-carboxamide (260 mg, 556 μmol, 1.00 eq) in DMF (4 mL) were added diphenyl(trifluoromethyl)sulfonium;trifluoromethanesulfonate ([Ph₂SCF₃]⁺[OTf]⁻, 450 mg, 1.11 mmol, 2.00 eq) and Cu (106 mg, 1.67 mmol, 3.00 eq) in portions under N₂. The reaction mixture was heated to 60° C. and stirred under N₂ for 9 h. After cooling to room temperature, the reaction mixture was poured into water (20 mL) and stirred for 3 min. The aqueous phase was extracted with ethyl acetate (3×20 mL). The combined organic phases were washed with brine (30 mL), dried over anhydrous Na₂SO₄, filtered, and concentrated under reduced pressure to give a residue. The residue was purified by prep-HPLC to give 1-methyl-N-((6-(thiazol-4-ylmethoxy)-3-(trifluoromethyl)-1H-indol-2-yl) methyl)cyclopropane-1-carboxamide (Compound 640). LC-MS (ESI) m/z calcd for C₁₉H₁₈F₃N₃O₂S: 409.11; found 409.8 [M+H]⁺. ¹H NMR (400 MHz, DMSO-d₆) δ 11.33 (s, 1H) 9.13 (d, J=1.6 Hz, 1H) 8.06 (t, J=5.6 Hz, 1H) 7.76 (d, J=1.6 Hz, 1H) 7.40 (d, J=8.4 Hz, 1H) 7.12 (d, J=2.0 Hz, 1H) 6.88 (dd, J=6.4, 2.1 Hz, 1H) 5.23 (s, 2H) 4.50 (d, J=5.2 Hz, 2H) 1.31 (s, 3H) 1.02-0.99 (m, 2H) 0.56-0.54 (m, 2H).

### Example 80 (Compound 641)

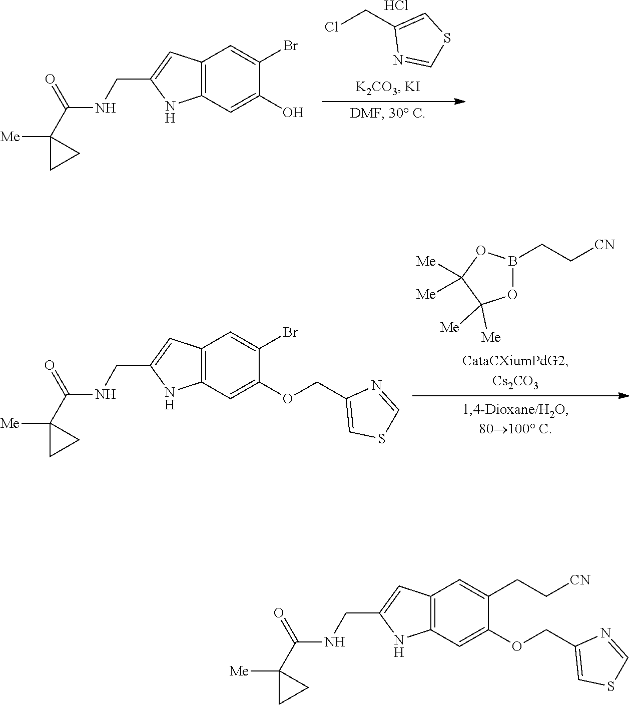 [SMILES: Cl ; CC1(C(=O)NCc2cc3cc(Br)c(O)cc3[nH]2)CC1 ; ClCc1cscn1 ; CC1(C(=O)NCc2cc3cc(Br)c(OCc4cscn4)cc3[nH]2)CC1 ; CC1(C)OB(CCC#N)OC1(C)C ; CC1(C(=O)NCc2cc3cc(CCC#N)c(OCc4cscn4)cc3[nH]2)CC1]

### Step 1: N-((5-bromo-6-(thiazol-4-ylmethoxy)-1H-indol-2-yl)methyl)-1-methylcyclopropane-1-carboxamide

To a mixture of N-((5-bromo-6-hydroxy-1H-indol-2-yl)methyl)-1-methylcyclopropane-1-carboxamide (200 mg, 433 μmol, 70.0% purity, 1.00 eq) and 4-(chloromethyl)thiazole hydrochloride (73.6 mg, 433 μmol, 1.00 eq) in DMF (1.0 mL) was added KI (71.9 mg, 433 μmol, 1.00 eq) and K₂CO₃ (240 mg, 1.73 mmol, 4.0 eq) under an atmosphere of N₂. The reaction mixture was warmed to 30° C. and stirred for 6 h. The reaction mixture was allowed to cool to room temperature and quenched by addition of H₂O (50 mL). The mixture was extracted with ethyl acetate (3×20 mL). The combined organic phases were washed with water (3×20 mL), dried over Na₂SO₄, and filtered. Concentration of the filtrate under reduced pressure gave N-((5-bromo-6-(thiazol-4-ylmethoxy)-1H-indol-2-yl)methyl)-1-methylcyclopropane-1-carboxamide which was used without further purification. LC-MS (ESI) m/z calcd for C₁₈H₁₈BrN₃O₂S: 419.03; found: 420.1 [M+H]⁺.

### Step 2: N-((5-(2-cyanoethyl)-6-(thiazol-4-ylmethoxy)-1H-indol-2-yl)methyl)-1-methylcyclopropane-1-carboxamide

A mixture of N-((5-bromo-6-(thiazol-4-ylmethoxy)-1H-indol-2-yl)methyl)-1-methylcyclopropane-1-carboxamide (100 mg, 238 μmol, 1.00 eq) and Cs₂CO₃ (155 mg, 476 μmol, 2.0 eq) in dioxane (5.0 mL) and H₂O (1.0 mL) was degassed and purged with nitrogen three times. To this mixture was added chloro[(di(1-adamantyl)-N-butylphosphine)-2-(2-aminobiphenyl)]palladium (15.9 mg, 23.8 μmol, 0.10 eq). The reaction mixture was warmed to 80° C., and then 3-(4,4,5,5-tetramethyl-1,3,2-dioxaborolan-2-yl)propanenitrile (51.7 mg, 285 μmol, 1.20 eq) was added dropwise. The reaction mixture was then warmed to 100° C. and stirred for 6 h. The reaction mixture was allowed to cool to room temperature and was quenched by addition of H₂O (50 mL) and extracted with ethyl acetate (3×20 mL). The combined organic phases were washed with brine (3×20 mL), dried over Na₂SO₄, filtered, and concentrated under reduced pressure. Purification by prep-HPLC gave N-((5-(2-cyanoethyl)-6-(thiazol-4-ylmethoxy)-1H-indol-2-yl)methyl)-1-methylcyclopropane-1-carboxamide (Compound 641). LC-MS (ESI) m/z calcd for C₂₁H₂₂N₄O₂S: 394.14; found: 395.3 [M+H]⁺. ¹H NMR (400 MHz, DMSO-d₆) δ 10.57 (s, 1H), 9.14 (d, J=2.0 Hz, 1H), 7.97 (t, J=6.0 Hz, 1H), 7.76 (s, 1H), 7.26 (s, 1H), 7.06 (s, 1H), 6.10 (s, 1H), 5.24 (s, 2H), 4.34 (d, J=5.6 Hz, 2H), 2.91-2.87 (m, 2H), 2.77-2.74 (m, 2H), 1.28 (s, 3H), 1.00-0.98 (m, 2H), 0.53-0.51 (m, 2H).

The following compounds in Table T-46 were synthesized using procedures similar to Compound 641 using the appropriate starting materials.

TABLE T-46

| | | | Exact | LCMS, |
|---|---|---|---|---|
| Compound | | | mass | found |
| No. | Structure | IUPAC name | (g/mol) | [M + H]⁺ |
| 642 | 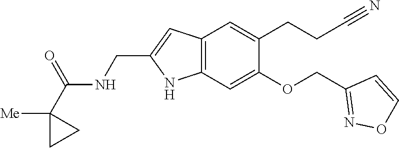 [SMILES: CC1(C(=O)NCc2cc3cc(CCC#N)c(OCc4ccon4)cc3[nH]2)CC1] | N-((5-(2-cyanoethyl)-6- (isoxazol-3-ylmethoxy)- 1H-indol-2-yl)methyl)- 1-methylcyclopropane- 1-carboxamide | 378.17 | 379.1 |

### Example 81 (Compound 643)

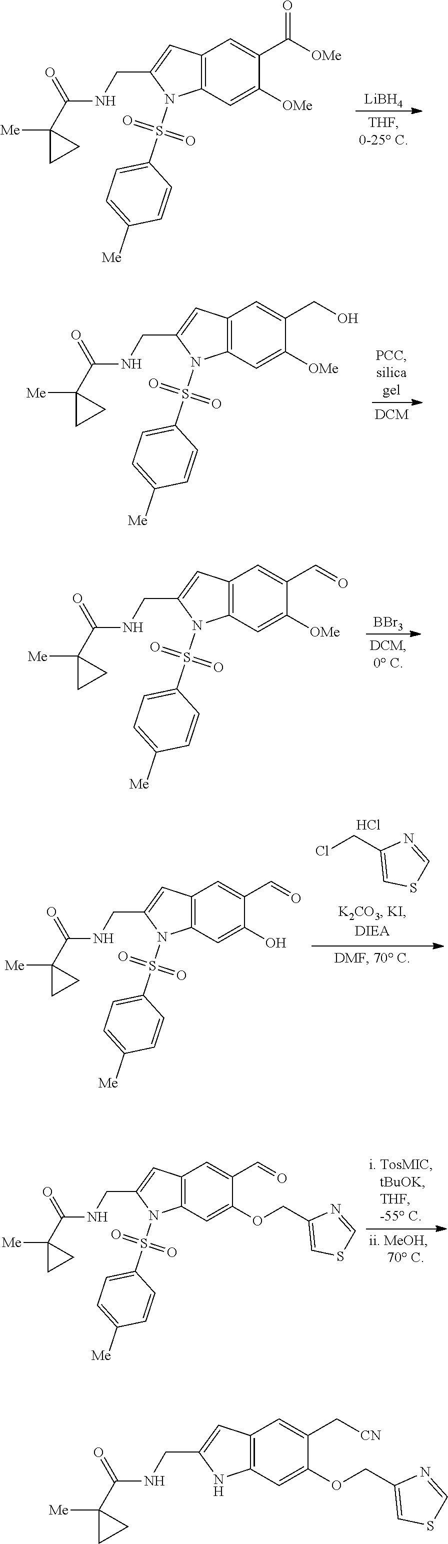

### Step 1: N-((5-(hydroxymethyl)-6-methoxy-1-tosyl-1H-indol-2-yl)methyl)-1-methylcyclopropane-1-carboxamide

A solution of methyl 2-(((tert-butoxycarbonyl)amino)methyl)-6-methoxy-1-tosyl-1H-indole-5-carboxylate (5.0 g, 10.6 mmol, 1.00 eq) in THF (50 mL) was degassed under vacuum and purged with N₂ three times and cooled to 0° C. LiBH₄ (2 M in THF, 21.2 mL, 4.00 eq) was added dropwise. The reaction mixture was warmed to 25° C. and stirred for 12 h. The reaction mixture was then added to sat. aq. NH₄Cl (60 mL) dropwise at 0° C. under N₂. The resulting mixture was extracted with EtOAc (3×80 mL). The combined organic layers were washed with brine (2×50 mL), dried over anhydrous Na₂SO₄, filtered, and concentrated under reduced pressure. The resulting crude residue was triturated with EtOAc (30 mL) at 25° C. for 10 min to give N-((5-(hydroxymethyl)-6-methoxy-1-tosyl-1H-indol-2-yl)methyl)-1-methylcyclopropane-1-carboxamide. LC-MS (ESI) m/z calcd for C₂₃H₂₆N₂O₅S: 442.16; found: 443.2 [M+H]⁺.

### Step 2: N-((5-formyl-6-methoxy-1-tosyl-1H-indol-2-yl)methyl)-1-methylcyclopropane-1-carboxamide

To a solution of N-((5-(hydroxymethyl)-6-methoxy-1-tosyl-1H-indol-2-yl)methyl)-1-methylcyclopropane-1-carboxamide (2.00 g, 4.52 mmol, 1.00 eq) in DCM (10 mL) were added PCC (1.46 g, 6.78 mmol, 1.5 eq) and silica gel (1 g). The mixture was stirred at 25° C. for 1 h. The reaction mixture was then filtered, and the filtrate was concentrated under reduced pressure. The resulting crude residue was triturated with EtOAc (10 mL) at 25° C. for 10 min to give N-((5-formyl-6-methoxy-1-tosyl-1H-indol-2-yl)methyl)-1-methylcyclopropane-1-carboxamide. LC-MS (ESI) m/z calcd for C₂₃H₂₄N₂O₅S: 440.14; found: 441.2 [M+H]⁺.

### Step 3: N-((5-formyl-6-hydroxy-1-tosyl-1H-indol-2-yl)methyl)-1-methylcyclopropane-1-carboxamide

To a solution of N-((5-formyl-6-methoxy-1-tosyl-1H-indol-2-yl)methyl)-1-methylcyclopropane-1-carboxamide (1.0 g, 2.27 mmol, 1.00 eq) in DCM (15 mL) was added BBr₃ (219 μL, 2.27 mmol, 1 eq) dropwise at 0° C. The reaction mixture was stirred at 0° C. for 2 h. The reaction mixture was warmed to 25° C. and poured into water (2 mL) at 0° C. Sat. aq. NaHCO₃ was added until pH=8. The resulting mixture was extracted with EtOAc (2×5 mL). The combined organic layers were washed with brine (10 mL), dried over anhydrous Na₂SO₄, filtered, and concentrated under reduced pressure. The resulting crude residue was triturated with EtOAc (30 mL) at 25° C. for 10 min to give N-((5-formyl-6-hydroxy-1-tosyl-1H-indol-2-yl)methyl)-1-methylcyclopropane-1-carboxamide. LC-MS (ESI) m/z calcd for C₂₂H₂₂N₂O₅S: 426.12; found: 427.4 [M+H]⁺.

### Step 4: N-((5-formyl-6-(thiazol-4-ylmethoxy)-1-tosyl-1H-indol-2-yl)methyl)-1-methylcyclopropane-1-carboxamide

To a mixture of N-((5-formyl-6-hydroxy-1-tosyl-1H-indol-2-yl)methyl)-1-methylcyclopropane-1-carboxamide (350 mg, 0.82 mmol, 1.00 eq) and 4-(chloromethyl)thiazole hydrochloride (120 mg, 0.90 mmol, 1.10 eq), and DIEA (159 mg, 1.23 mmol, 1.50 eq) in DMF (2 mL) were added K₂CO₃ (227 mg, 1.64 mmol, 2.00 eq), and KI (69.0 mg, 0.41 mmol, 0.5 eq). The reaction mixture was then heated to 70° C. for 2 h. The reaction mixture was cooled to 25° C. and poured into water (10 mL). The resulting mixture was stirred for 10 min and extracted with EtOAc (3×10 mL). The combined organic layers were washed with brine (3×10 mL), dried over anhydrous Na₂SO₄, filtered, and concentrated under reduced pressure. The resulting crude residue was triturated with MTBE (5 mL) at 25° C. for 10 min to give N-((5-formyl-6-(thiazol-4-ylmethoxy)-1-tosyl-1H-indol-2-yl)methyl)-1-methylcyclopropane-1-carboxamide. LC-MS (ESI) m/z calcd for C₂₆H₂₅N₃O₅S₂: 523.12; found: 524.4 [M+H]⁺.

### Step 5: N-((5-(cyanomethyl)-6-(thiazol-4-ylmethoxy)-1H-indol-2-yl)methyl)-1-methylcyclopropane-1-carboxamide

A solution of tBuOK (1 M in THF, 802.1 μL, 0.80 mmol, 2.00 eq) in THF (5 mL) was cooled to −55° C. and a solution of 1-(isocyanomethylsulfonyl)-4-methyl-benzene (210 mg, 0.44 mmol, 1.10 eq) in THF (5 mL) was added dropwise and the resulting mixture was stirred for 15 min at −55° C. A solution of N-((5-formyl-6-(thiazol-4-ylmethoxy)-1-tosyl-1H-indol-2-yl)methyl)-1-methylcyclopropane-1-carboxamide (210 mg, 0.40 mmol, 1.00 eq) in THF (5 mL) was then added dropwise and the resulting reaction mixture was stirred at −55° C. for 2 h. MeOH (16.2 μL, 0.40 mmol, 1.00 eq) was then added and the reaction mixture was heated to 70° C. for 30 min. The reaction mixture was cooled to 25° C. and concentrated in vacuo. The resulting residue was diluted with water (10 mL) and extracted with EtOAc (3×10 mL). The combined organic layers were washed with brine (2×10 mL), dried over anhydrous Na₂SO₄, filtered, and concentrated under reduced pressure. Purification by prep-HPLC gave N-((5-(cyanomethyl)-6-(thiazol-4-ylmethoxy)-1H-indol-2-yl)methyl)-1-methylcyclopropane-1-carboxamide (Compound 643). LC-MS (ESI) m/z calcd for C₂₀H₂₀N₄O₂S: 380.13; found: 381.1[M+H]⁺. ¹H NMR (400 MHz, DMSO-d₆) 10.67 (s, 1H), 9.15 (d, J=1.6 Hz, 1H), 8.00 (t, J=6.0 Hz, 1H), 7.79 (s, 1H), 7.40 (s, 1H), 7.12 (s, 1H), 6.15 (s, 1H), 5.30 (s, 2H), 4.35 (d, J=6.0 Hz, 2H), 3.90 (s, 2H), 1.28 (s, 3H), 1.00-0.98 (m, 2H), 0.54-0.51 (m, 2H).

The following compounds in Table T-47 were synthesized using procedures similar to Compound 643 using the appropriate starting materials.

TABLE T-47

| | | | Exact | LCMS, |
|---|---|---|---|---|
| Compound | | | mass | found |
| No. | Structure | IUPAC name | (g/mol) | [M + H]⁺ |
| 644 | 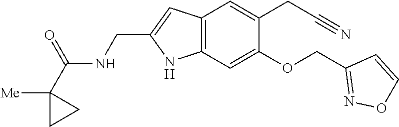 [SMILES: CC1(C(=O)NCc2cc3cc(CC#N)c(OCc4ccon4)cc3[nH]2)CC1] | N-((5-(cyanomethyl)-6- (isoxazol-3-ylmethoxy)- 1H-indol-2-yl)methyl)- 1-methylcyclopropane- 1-carboxamide | 364.15 | 365.2 |

### Example 82 (Compound 645)

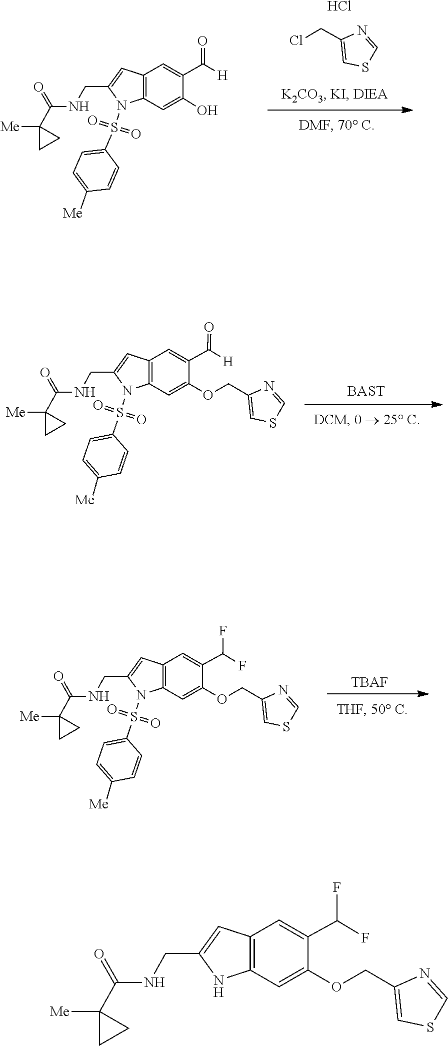

### Step 1: N-((5-formyl-6-(thiazol-4-ylmethoxy)-1-tosyl-1H-indol-2-yl)methyl)-1-methylcyclopropane-1-carboxamide

To a solution of N-((5-formyl-6-hydroxy-1-tosyl-1H-indol-2-yl)methyl)-1-methylcyclopropane-1-carboxamide (150 mg, 352 μmol, 1.00 eq) in DMF (1.5 mL) was added 4-(chloromethyl)thiazole hydrochloride (65.8 mg, 387 μmol, 1.10 eq, HCl), K₂CO₃ (97.2 mg, 703 μmol, 2.00 eq), KI (29.2 mg, 176 μmol, 0.50 eq), and DIEA (68.2 mg, 528 μmol, 1.50 eq) sequentially. The reaction mixture was heated to 70° C. After 2 h, the reaction was quenched with H₂O (10 mL) at 0° C. The solution was extracted with EtOAc (3×10 mL). The combined organic layers were washed with brine (10 mL), dried over anhydrous Na₂SO₄, filtered, and concentrated under reduced pressure. Purification by column chromatography gave N-((5-formyl-6-(thiazol-4-ylmethoxy)-1-tosyl-1H-indol-2-yl)methyl)-1-methylcyclopropane-1-carboxamide (200 mg). LC-MS (ESI) m/z calcd for C₂₆H₂₅N₃O₅S₂: 523.12; found 524.2 [M+H]⁺.

### Step 2: N-((5-(difluoromethyl)-6-(thiazol-4-ylmethoxy)-1-tosyl-1H-indol-2-yl)methyl)-1-methylcyclopropane-1-carboxamide

To a solution of N-((5-formyl-6-(thiazol-4-ylmethoxy)-1-tosyl-1H-indol-2-yl)methyl)-1-methylcyclopropane-1-carboxamide (150 mg, 286 μmol, 1.00 eq) in DCM (5 mL) was added 1,1,1-trifluoro-N,N-bis(2-methoxyethyl)-14-sulfinimine (BAST, 380 mg, 1.72 mmol, 6.00 eq) dropwise at 0° C. The reaction was warmed to 25° C. After 16 h, the reaction mixture was quenched with H₂O (10 mL) at 0° C. The solution was extracted with EtOAc (3×15 mL). The combined organic layers were washed with brine (2×10 mL), dried over anhydrous Na₂SO₄, filtered, and concentrated under reduced pressure. Purification by column chromatography gave N-((5-(difluoromethyl)-6-(thiazol-4-ylmethoxy)-1-tosyl-1H-indol-2-yl)methyl)-1-methylcyclopropane-1-carboxamide (30.0 mg). LC-MS (ESI) m/z calcd for C₂₆H₂₅F₂N₃O₄S₂: 545.15; found 546.2 [M+H]⁺.

### Step 3: N-((5-(difluoromethyl)-6-(thiazol-4-ylmethoxy)-1H-indol-2-yl)methyl)-1-methylcyclopropane-1-carboxamide

To a solution of N-((5-(difluoromethyl)-6-(thiazol-4-ylmethoxy)-1-tosyl-1H-indol-2-yl)methyl)-1-methylcyclopropane-1-carboxamide (30.0 mg, 55.0 μmol, 1.00 eq) in THF (0.5 mL) was added TBAF (1 M in THF, 165 uL, 3.00 eq) dropwise. The reaction mixture was heated to 50° C. After 2 h, the reaction mixture was cooled to room temperature and quenched with H₂O (5 mL). The reaction mixture was extracted with EtOAc (3×10 mL). The combined organic layers were washed with brine (3×10 mL), dried over anhydrous Na₂SO₄, filtered, and concentrated under reduced pressure. Purification by prep-TLC gave N-((5-(difluoromethyl)-6-(thiazol-4-ylmethoxy)-1H-indol-2-yl)methyl)-1-methylcyclopropane-1-carboxamide (Compound 645). LC-MS (ESI) m/z calcd for C₁₉H₁₉F₂N₃O₂S: 391.12; found 372.2 [M−F]⁺. ¹H NMR (400 MHz, DMSO-d₆) δ 10.86 (s, 1H), 9.14 (s, 1H), 8.03 (t, J=6.0 Hz, 1H), 7.77 (s, 1H), 7.60 (s, 1H), 7.27-6.99 (m, 2H), 6.23 (s, 1H), 5.29 (s, 2H), 4.37 (d, J=6.0 Hz, 2H), 1.29 (s, 3H), 1.01-0.98 (m, 2H), 0.54-0.52 (m, 2H).

The following compounds in Table T-48 were synthesized using procedures similar to Compound 645 using the appropriate starting materials.

TABLE T-48

| | | | Exact | LCMS, |
|---|---|---|---|---|
| Compound | | | mass | found |
| No. | Structure | IUPAC name | (g/mol) | [M + H]⁺ |
| 646 | 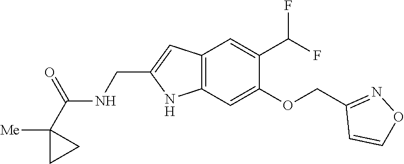 [SMILES: CC1(C(=O)NCc2cc3cc(C(F)F)c(OCc4ccon4)cc3[nH]2)CC1] | N-((5-(difluoromethyl)- 6-(isoxazol-3- ylmethoxy)-1H-indol-2- yl)methyl)-1- methylcyclopropane-1- carboxamide | 375.14 | 356.2 [M − F]⁺ |
| 647 | 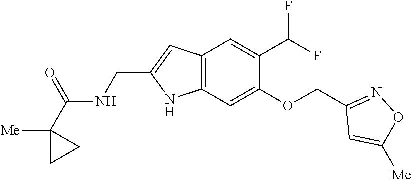 [SMILES: Cc1cc(COc2cc3[nH]c(CNC(=O)C4(C)CC4)cc3cc2C(F)F)no1] | N-((5-(difluoromethyl)- 6-((5-methylisoxazol-3- yl)methoxy)-1H-indol- 2-yl)methyl)-1- methylcyclopropane-1- carboxamide | 389.16 | 370.3 [M − F]⁺ |

### Example 83 (Compound 648)

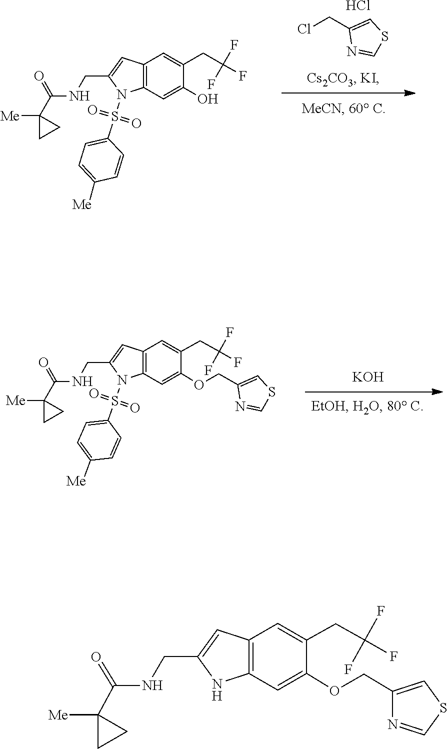 [SMILES: Cl ; Cc1ccc(S(=O)(=O)n2c(CNC(=O)C3(C)CC3)cc3cc(CC(F)(F)F)c(O)cc32)cc1 ; ClCc1cscn1 ; Cc1ccc(S(=O)(=O)n2c(CNC(=O)C3(C)CC3)cc3cc(CC(F)(F)F)c(OCc4cscn4)cc32)cc1 ; "KOH" ; CC1(C(=O)NCc2cc3cc(CC(F)(F)F)c(OCc4cscn4)cc3[nH]2)CC1]

### Step 1: 1-methyl-N-((6-(thiazol-4-ylmethoxy)-1-tosyl-5-(2,2,2-trifluoroethyl)-1H-indol-2-yl)methyl)cyclopropane-1-carboxamide

To a solution of N-((6-hydroxy-1-tosyl-5-(2,2,2-trifluoroethyl)-1H-indol-2-yl)methyl)-1-methylcyclopropane-1-carboxamide (400 mg, 624 μmol, 75% purity, 1.00 eq) and 4-(chloromethyl)thiazole (127 mg, 749 μmol, 1.20 eq, HCl salt) in MeCN (6 mL) was added KI (104 mg, 624 μmol, 1.00 eq) and Cs₂CO₃ (407 mg, 1.25 mmol, 2.00 eq). The reaction mixture was warmed to 60° C. and stirred for 16 h. The reaction mixture was allowed to cool to room temperature, was diluted with water (60 mL), and was extracted with EtOAc (3×20 mL). The combined organic phases were washed with brine (20 mL), dried over Na₂SO₄, filtered, and concentrated under reduced pressure. Purification by column chromatography gave 1-methyl-N-((6-(thiazol-4-ylmethoxy)-1-tosyl-5-(2,2,2-trifluoroethyl)-1H-indol-2-yl)methyl)cyclopropane-1-carboxamide. LC-MS (ESI) m/z calcd for C₂₇H₂₆F₃N₃O₄S₂: 577.13; found 578.3 [M+H]⁺.

### Step 2: 1-methyl-N-((6-(thiazol-4-ylmethoxy)-5-(2,2,2-trifluoroethyl)-1H-indol-2-yl)methyl)cyclopropane-1-carboxamide

To a solution of 1-methyl-N-((6-(thiazol-4-ylmethoxy)-1-tosyl-5-(2,2,2-trifluoroethyl)-1H-indol-2-yl)methyl)cyclopropane-1-carboxamide (200 mg, 346 μmol, 1.00 eq) in EtOH (10 mL) and H₂O (5 mL) was added KOH (388 mg, 6.92 mmol, 20.0 eq). The reaction mixture was warmed to 80° C. and stirred for 16 h. The reaction mixture was allowed to cool to room temperature, was diluted with water (45 mL), and was extracted with EtOAc (3×15 mL). The combined organic phases were washed with brine (15 mL), dried over Na₂SO₄, filtered, and concentrated under reduced pressure. Purification by prep-HPLC gave 1-methyl-N-[[6-(thiazol-4-ylmethoxy)-5-(2,2,2-trifluoroethyl)-1H-indol-2-yl]methyl] cyclopropanecarboxamide (Compound 648). LC-MS (ESI) m/z calcd for C₂₀H₂₀F₃N₃O₂S: 423.12; found 424.2 [M+H]⁺. ¹H NMR (400 MHz, DMSO-d₆) δ 10.66 (s, 1H), 9.14 (s, 1H), 7.98 (t, J=6.0 Hz, 1H), 7.69 (d, J=1.2 Hz, 1H), 7.36 (s, 1H), 7.11 (s, 1H), 6.13 (s, 1H), 5.25 (s, 2H), 4.35 (d, J=5.6 Hz, 2H), 3.68-3.55 (m, 2H), 1.28 (s, 3H), 1.00-0.98 (m, 2H), 0.54-0.51 (m, 2H).

The following compounds in Table T-49 were synthesized using procedures similar to Compound 648 using the appropriate starting materials.

TABLE T-49

| | | | Exact | LCMS, |
|---|---|---|---|---|
| Compound | | | mass | found |
| No. | Structure | IUPAC name | (g/mol) | [M + H]⁺ |
| 649 | 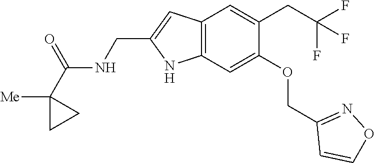 [SMILES: CC1(C(=O)NCc2cc3cc(CC(F)(F)F)c(OCc4ccon4)cc3[nH]2)CC1] | N-((6-(isoxazol-3- ylmethoxy)-5-(2,2,2- trifluoroethyl)-1H- indol-2-yl)methyl)-1- methylcyclopropane-1- carboxamide | 407.15 | 408.1 |
| 650 | 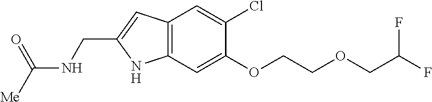 [SMILES: CC(=O)NCc1cc2cc(Cl)c(OCCOCC(F)F)cc2[nH]1] | N-((5-chloro-6-(2-(2,2- difluoroethoxy)ethoxy)- 1H-indol-2- yl)methyl)acetamide | 346.09 | 288.1 [M − CH₃CO NH + H]+ |

### Example 84 (Compound 651)

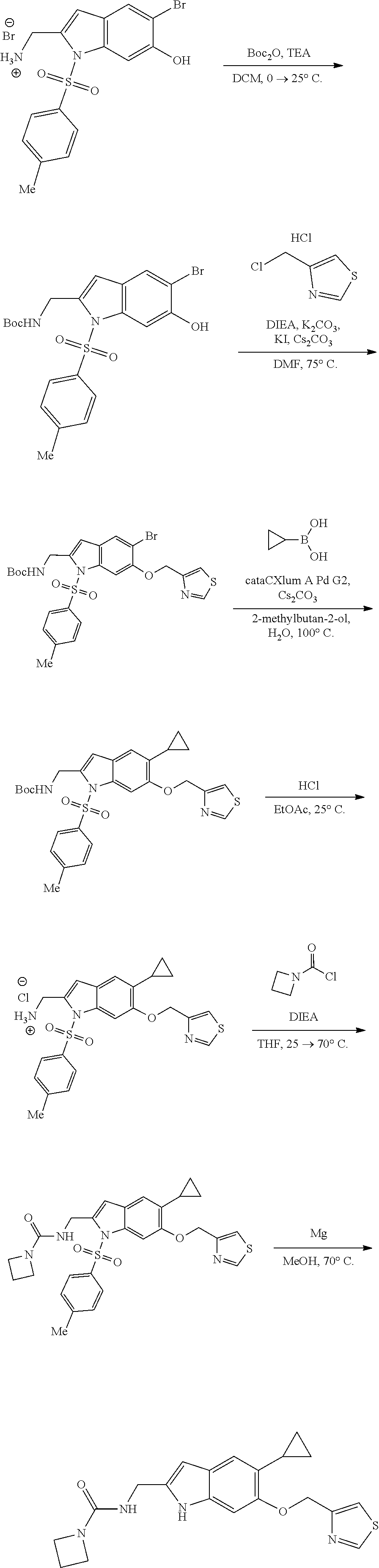

### Step 1: tert-butyl-((5-bromo-6-hydroxy-1-tosyl-1H-indol-2-yl)methyl)carbamate

To a solution of 2-(aminomethyl)-5-bromo-1-(p-tolylsulfonyl)indol-6-ol (2.00 g, 4.20 mmol, 1.00 eq, HBr) in DCM (20 mL) were added TEA (637 mg, 6.30 mmol, 1.50 eq) and Boc₂O (1.10 g, 5.04 mmol, 1.20 eq) in one portion at 0° C. The reaction mixture was stirred at 25° C. for 1 h. The reaction mixture was diluted with H₂O (100 mL) and extracted with ethyl acetate (50 mL×3). The combined organic layers were washed with brine (50 mL), dried over anhydrous Na₂SO₄, filtered, and concentrated under reduced pressure to give a residue. The residue was purified by column chromatography to give tert-butyl ((5-bromo-6-hydroxy-1-tosyl-1H-indol-2-yl)methyl)carbamate. LC-MS (ESI) m/z calcd for C₂₁H₂₃BrN₂O₅S: 494.05; found: 517.0 [M+Na]⁺.

### Step 2: tert-butyl ((5-bromo-6-(thiazol-4-ylmethoxy)-1-tosyl-1H-indol-2-yl)methyl)carbamate

A mixture of tert-butyl ((5-bromo-6-hydroxy-1-tosyl-1H-indol-2-yl) methyl) carbamate (600 mg, 1.21 mmol, 1.00 eq), 4-(chloromethyl)thiazole (205 mg, 1.21 mmol, 1.00 eq, HCl salt), K₂CO₃ (334 mg, 2.42 mmol, 2.00 eq), KI (201 mg, 1.21 mmol, 1.00 eq), Cs₂CO₃ (394 mg, 1.21 mmol, 1.00 eq) and DIEA (234 mg, 1.82 mmol, 1.50 eq) in DMF (12 mL) was degassed and purged with N₂ for 3 times, and then the reaction mixture was heated and stirred at 75° C. for 2 h under N₂ atmosphere. After cooling to room temperature, the reaction mixture was diluted with H₂O (100 mL) and extracted with ethyl acetate (50 mL×3). The combined organic layers were washed with brine (50 mL), dried over anhydrous Na₂SO₄, filtered, and concentrated under reduced pressure to give a residue. The residue was purified by column chromatography to give tert-butyl ((5-bromo-6-(thiazol-4-ylmethoxy)-1-tosyl-1H-indol-2-yl)methyl)carbamate. LC-MS (ESI) m/z calcd for C₂₅H₂₆BrN₃O₅S₂: 591.05; found: 536.0 [M−tBu+H]⁺.

### Step 3: tert-butyl ((5-cyclopropyl-6-(thiazol-4-ylmethoxy)-1-tosyl-1H-indol-2-yl)methyl)carbamate

To a solution of tert-butyl ((5-bromo-6-(thiazol-4-ylmethoxy)-1-tosyl-1H-indol-2-yl)methyl)carbamate (680 mg, 1.15 mmol, 1.00 eq) in 2-methylbutan-2-ol (10 mL) and H₂O (2 mL) were added Cs₂CO₃ (747 mg, 2.30 mmol, 2.00 eq) and cyclopropylboronic acid (492 mg, 5.74 mmol, 5.00 eq) in one portion, then [2-(2-aminophenyl)phenyl]-chloro-palladium;bis(1-adamantyl)-butyl-phosphane (cataCXium A Pd G2, 76.7 mg, 114 μmol, 0.10 eq) was added in one portion under N₂. The reaction mixture was heated and stirred at 100° C. for 16 h. After cooling to room temperature, the reaction mixture was diluted with H₂O (100 mL) and extracted with ethyl acetate (50 mL×3). The combined organic layers were washed with brine (50 mL), dried over anhydrous Na₂SO₄, filtered, and concentrated under reduced pressure to give a residue. The residue was purified by column chromatography to give tert-butyl ((5-cyclopropyl-6-(thiazol-4-ylmethoxy)-1-tosyl-1H-indol-2-yl)methyl)carbamate. LC-MS (ESI) m/z calcd for C₂₈H₃₁N₃O₅S₂: 553.17; found: 576.2 [M+Na]⁺.

### Step 4: (5-cyclopropyl-6-(thiazol-4-ylmethoxy)-1-tosyl-1H-indol-2-yl)methanaminium chloride

To a solution of tert-butyl ((5-cyclopropyl-6-(thiazol-4-ylmethoxy)-1-tosyl-1H-indol-2-yl)methyl)carbamate (680 mg, 1.23 mmol, 1.00 eq) in EtOAc (1 mL) was added HCl in EtOAc (4 M, 10 mL, 32.6 eq) in portions. The reaction mixture was stirred at 25° C. for 1 h. The reaction mixture was concentrated under reduced pressure to give a residue. The residue was slurried with MTBE (20 mL) for 30 mins and then filtered, and the filter cake was collected to give (5-cyclopropyl-6-(thiazol-4-ylmethoxy)-1-tosyl-1H-indol-2-yl)methanaminium chloride. LC-MS (ESI) m/z calcd for C₂₃H₂₃N₃O₃S₂: 453.12; found: 437.1 [M−NH₂+H]⁺.

### Step 5: N-((5-cyclopropyl-6-(thiazol-4-ylmethoxy)-1-tosyl-1H-indol-2-yl)methyl)azetidine-1-carboxamide

To a solution of (5-cyclopropyl-6-(thiazol-4-ylmethoxy)-1-tosyl-1H-indol-2-yl)methanaminium chloride (200 mg, 1.00 eq) in THF (3 mL) were added DIEA (263 mg, 2.04 mmol, 5.00 eq) and azetidine-1-carbonyl chloride (146 mg, 1.22 mmol, 3.00 eq) in one portion at 25° C. The reaction mixture was heated to 70° C. and stirred for 2 h. After cooling to room temperature, the reaction mixture was diluted with H₂O (20 mL) and extracted with EtOAc (15×3 mL). The combined organic layers were washed with brine (50 mL), dried over anhydrous Na₂SO₄, filtered, and concentrated under reduced pressure to give N-((5-cyclopropyl-6-(thiazol-4-ylmethoxy)-1-tosyl-1H-indol-2-yl)methyl)azetidine-1-carboxamide, which was used for next step directly without of further purification. LC-MS (ESI) m/z calcd for C₂₇H₂₈N₄O₄S₂: 536.16; found: 537.2 [M+H]⁺.

### Step 6: N-((5-cyclopropyl-6-(thiazol-4-ylmethoxy)-1H-indol-2-yl)methyl)azetidine-1-carboxamide

To a solution of N-((5-cyclopropyl-6-(thiazol-4-ylmethoxy)-1-tosyl-1H-indol-2-yl)methyl)azetidine-1-carboxamide (130 mg, 242 μmol, 1.00 eq) in MeOH (2 mL) was added Mg (156 mg, 6.42 mmol, 26.5 eq) in one portion. The reaction mixture was heated to 70° C. and stirred for 2 h. After cooling to room temperature, the mixture was adjusted to pH=7 with aqueous HCl (1 M) solution and extracted with EtOAc (50×3 mL). The combined organic phase was washed with brine (50 mL), dried with anhydrous Na₂SO₄, filtered, and concentrated under reduced pressure to give a residue. The residue was purified by prep-HPLC to give N-((5-cyclopropyl-6-(thiazol-4-ylmethoxy)-1H-indol-2-yl)methyl)azetidine-1-carboxamide (Compound 651). LC-MS (ESI) m/z calcd for C₂₀H₂₂N₄O₂S: 382.15; found: 382.9 [M+H]⁺. 1H NMR (400 MHz, DMSO-d₆) δ 10.45 (s, 1H), 9.14 (d, J=2.0 Hz, 1H), 7.74 (d, J=2.0 Hz, 1H), 6.99 (s, 1H), 6.92 (s, 1H), 6.67 (t, J=5.8 Hz, 1H), 6.03 (d, J=1.0 Hz, 1H), 5.22 (s, 2H), 4.23 (d, J=5.8 Hz, 2H), 3.81 (t, J=5.8 Hz, 4H), 2.16-2.08 (m, 3H), 0.84-0.82 (m, 2H), 0.57-0.55 (m, 2H).

The following compounds in Table T-50 were synthesized using procedures similar to Compound 651 using the appropriate starting materials.

TABLE T-50

| | | | Exact | LCMS, |
|---|---|---|---|---|
| Compound | | | mass | found |
| No. | Structure | IUPAC name | (g/mol) | [M + H]⁺ |
| 652 | 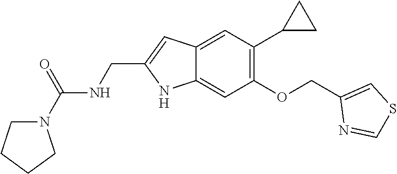 [SMILES: O=C(NCc1cc2cc(C3CC3)c(OCc3cscn3)cc2[nH]1)N1CCCC1] | N-((5-cyclopropyl-6- (thiazol-4-ylmethoxy)- 1H-indol-2- yl)methyl)pyrrolidine-1- carboxamide | 396.16 | 396.9 |
| 653 | 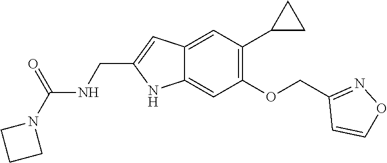 [SMILES: O=C(NCc1cc2cc(C3CC3)c(OCc3ccon3)cc2[nH]1)N1CCC1] | N-((5-cyclopropyl-6- (isoxazol-3-ylmethoxy)- 1H-indol-2- yl)methyl)azetidine-1- carboxamide | 366.17 | 367.4 |
| 654 | 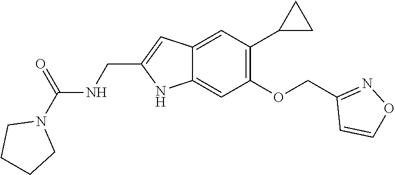 [SMILES: O=C(NCc1cc2cc(C3CC3)c(OCc3ccon3)cc2[nH]1)N1CCCC1] | N-((5-cyclopropyl-6- (isoxazol-3-ylmethoxy)- 1H-indol-2- yl)methyl)pyrrolidine-1- carboxamide | 380.18 | 381.4 |

### Example 85 (Compound 655)

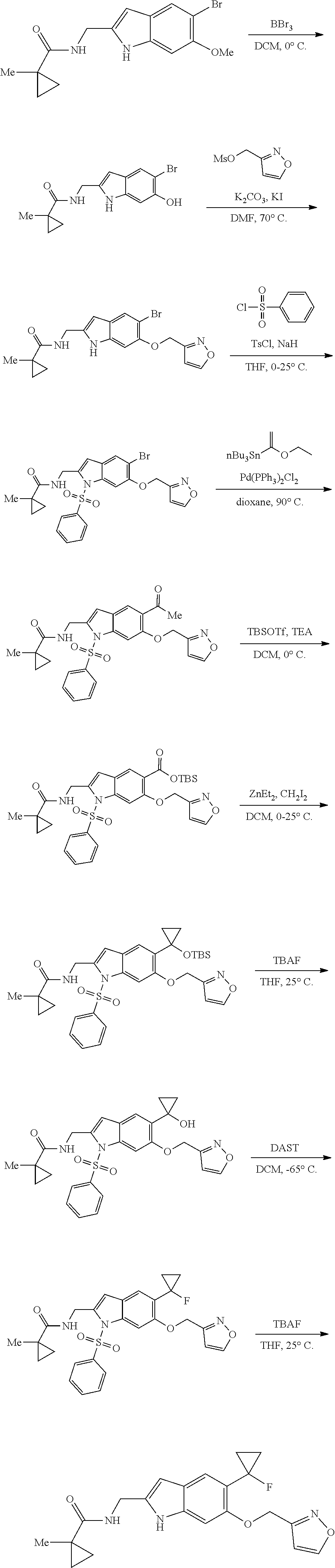

### Step 1: N-((5-bromo-6-hydroxy-1H-indol-2-yl)methyl)-1-methylcyclopropane-1-carboxamide

To a solution of N-((5-bromo-6-methoxy-1H-indol-2-yl)methyl)-1-methylcyclopropane-1-carboxamide (5.00 g, 14.8 mmol, 1.00 eq) in DCM (50.0 mL) at 0° C. was added BBr₃ (59.3 mmol, 5.71 mL, 4.00 eq) dropwise. The reaction was allowed to proceed at 0° C. After 1 h, the reaction was quenched with H₂O (200 mL) at 0° C. The pH was adjusted to 8 with saturated aqueous NaHCO₃. The solution was extracted with DCM (3×100 mL). The combined organic layers were dried over anhydrous Na₂SO₄, filtered, and concentrated under reduced pressure. The residue was triturated with ethyl acetate (15 mL) at 25° C. for 2 min. The resultant solids were collected by vacuum filtration, washed with ethyl acetate (5 mL), and dried under reduced pressure to give N-((5-bromo-6-hydroxy-1H-indol-2-yl)methyl)-1-methylcyclopropane-1-carboxamide. LC-MS (ESI) m/z calcd for C₁₄H₁₅BrN₂O₂: 322.03; found: 323.0 [M+H]⁺.

### Step 2: N-((5-bromo-6-(isoxazol-3-ylmethoxy)-1H-indol-2-yl)methyl)-1-methylcyclopropane-1-carboxamide

To a solution of N-((5-bromo-6-hydroxy-1H-indol-2-yl)methyl)-1-methylcyclopropane-1-carboxamide (2.00 g, 6.19 mmol, 1.00 eq), in DMF (20 mL) was added isoxazol-3-ylmethyl methanesulfonate (1.10 g, 6.19 mmol, 1.00 eq), K₂CO₃ (1.71 g, 12.4 mmol, 2.00 eq), and KI (513 mg, 3.09 mmol, 0.50 eq). The reaction mixture was heated to 70° C. After 1 h, the reaction mixture was poured into water (20 mL) at 25° C. After 5 min, the solution was extracted with ethyl acetate (2×20 mL). The combined organic layers were washed with brine (2×20 mL), dried over anhydrous Na₂SO₄, filtered, and concentrated under reduced pressure. Purification by column chromatography gave N-((5-bromo-6-(isoxazol-3-ylmethoxy)-1H-indol-2-yl)methyl)-1-methylcyclopropane-1-carboxamide. LC-MS (ESI) m/z calcd for C₁₈H₁₈BrN₃O₃: 403.05; found: 404.1 [M+H]⁺.

### Step 3: N-((5-bromo-6-(isoxazol-3-ylmethoxy)-1-(phenylsulfonyl)-1H-indol-2-yl)methyl)-1-methylcyclopropane-1-carboxamide

To a solution of N-((5-bromo-6-(isoxazol-3-ylmethoxy)-1H-indol-2-yl)methyl)-1-methylcyclopropane-1-carboxamide (1.96 g, 4.85 mmol, 1.00 eq) in THF (20 mL) at 0° C. was added NaH (388 mg, 9.70 mmol, 2.00 eq) in portions. The reaction was allowed to proceed at 0° C. After 30 min, benzenesulfonyl chloride (858 mg 4.85 mmol, 1.00 eq) was added in portions. The reaction was allowed to warm to 25° C. After 1 h, the reaction was poured into water (20 mL). The solution was extracted with ethyl acetate (2×20 mL). The combined organic layers were washed with brine (2×20 mL), dried over anhydrous Na₂SO₄, filtered, and concentrated under reduced pressure. The residue was triturated with MTBE (20 mL) at 25° C. After 30 min the resultant solids were collected by vacuum filtration to give N-((5-bromo-6-(isoxazol-3-ylmethoxy)-1-(phenylsulfonyl)-1H-indol-2-yl)methyl)-1-methylcyclopropane-1-carboxamide. LC-MS (ESI) m/z calcd for C₂₄H₂₂BrN₃O₅S: 543.05; found: 566.1 [M+Na]⁺.

### Step 4: N-((5-acetyl-6-(isoxazol-3-ylmethoxy)-1-(phenylsulfonyl)-1H-indol-2-yl)methyl)-1-methylcyclopropane-1-carboxamide

To a solution of N-((5-bromo-6-(isoxazol-3-ylmethoxy)-1-(phenylsulfonyl)-1H-indol-2-yl)methyl)-1-methylcyclopropane-1-carboxamide (1.00 g, 1.84 mmol, 1.00 eq) in dioxane (10 mL) was added tributyl(1-ethoxyvinyl)stannane (3.67 mmol, 1.24 mL, 2.00 eq) and bis(triphenylphosphine)palladium(II) dichloride (129 mg, 184 μmol, 0.10 eq). The reaction mixture was degassed and purged with N₂ three times then heated to 90° C. After 16 h, the reaction was quenched by addition of saturated aqueous NH₄Cl (5 mL) at 25° C. The reaction mixture was extracted with ethyl acetate (3×15 mL). The combined organic layers were washed with brine (2×15 mL), dried over Na₂SO₄, filtered, and concentrated under reduced pressure. Purification by column chromatography gave N-((5-acetyl-6-(isoxazol-3-ylmethoxy)-1-(phenylsulfonyl)-1H-indol-2-yl)methyl)-1-methylcyclopropane-1-carboxamide. LC-MS (ESI) m/z calcd for C₂₆H₂₅N₃O₆S: 507.15; found: 508.2 [M+H]⁺.

### Step 5: N-((5-(1-((tert-butyldimethylsilyl)oxy)vinyl)-6-(isoxazol-3-ylmethoxy)-1-(phenylsulfonyl)-1H-indol-2-yl)methyl)-1-methylcyclopropane-1-carboxamide

To a solution of N-((5-acetyl-6-(isoxazol-3-ylmethoxy)-1-(phenylsulfonyl)-1H-indol-2-yl)methyl)-1-methylcyclopropane-1-carboxamide (700 mg, 1.38 mmol, 1.00 eq) in DCM (7 mL) at 0° C. was added triethylamine (TEA, 348 mg 3.45 mmol, 2.50 eq) and tert-butyldimethylsilyl trifluoromethanesulfonate (730 mg 2.76 mmol, 635 μL, 2.00 eq) in portions. The reaction was allowed to proceed at 0° C. After 1 h, the reaction mixture was quenched by addition of H₂O (10 mL) at 25° C. The reaction mixture was extracted with ethyl acetate (3×15 mL). The combined organic layers were washed with brine (2×15 mL), dried over anhydrous Na₂SO₄, filtered, and concentrated under reduced pressure to give N-((5-(1-((tert-butyldimethylsilyl)oxy)vinyl)-6-(isoxazol-3-ylmethoxy)-1-(phenylsulfonyl)-1H-indol-2-yl)methyl)-1-methylcyclopropane-1-carboxamide, which was used directly without further purification. LC-MS (ESI) m/z calcd for C₃₂H₃₉N₃O₆SSi: 621.23; found: 622.3 [M+H]⁺.

### Step 6: N-((5-(1-((tert-butyldimethylsilyl)oxy)cyclopropyl)-6-(isoxazol-3-ylmethoxy)-1-(phenylsulfonyl)-1H-indol-2-yl)methyl)-1-methylcyclopropane-1-carboxamide

To a solution of ZnEt₂ (1 M in toluene, 10.3 mL, 8.00 eq) in toluene was added a solution of CH₂I₂ (2.76 g, 10.3 mmol, 8.00 eq) in DCM (4 mL) dropwise at 0° C. The mixture was stirred at 0° C. After 30 minutes N-((5-(1-((tert-butyldimethylsilyl)oxy)vinyl)-6-(isoxazol-3-ylmethoxy)-1-(phenylsulfonyl)-1H-indol-2-yl)methyl)-1-methylcyclopropane-1-carboxamide (800 mg, 1.29 mmol, 1.00 eq) in DCM (4 mL) was added dropwise. The reaction was allowed to warm to 25° C. After 16 h the reaction was poured into water (30 mL). The reaction mixture was extracted with ethyl acetate (3×10 mL). The combined organic layers were washed with brine (10 mL), dried over anhydrous Na₂SO₄, filtered, and concentrated under reduced pressure to give N-((5-(1-((tert-butyldimethylsilyl)oxy)cyclopropyl)-6-(isoxazol-3-ylmethoxy)-1-(phenylsulfonyl)-1H-indol-2-yl)methyl)-1-methylcyclopropane-1-carboxamide, which was used directly without further purification. LC-MS (ESI) m/z calcd for C₃₃H₄₁N₃O₆SSi: 635.25; found: 658.4 [M+Na]⁺.

### Step 7: N-((5-(1-hydroxycyclopropyl)-6-(isoxazol-3-ylmethoxy)-1-(phenylsulfonyl)-1H-indol-2-yl)methyl)-1-methylcyclopropane-1-carboxamide

To a solution of N-((5-(1-((tert-butyldimethylsilyl)oxy)cyclopropyl)-6-(isoxazol-3-ylmethoxy)-1-(phenylsulfonyl)-1H-indol-2-yl)methyl)-1-methylcyclopropane-1-carboxamide (720 mg, 1.13 mmol, 1.00 eq) in THF (0.2 mL) was added TBAF (1 M in THF, 11.32 mL, 10.0 eq) dropwise. After 16 h, the reaction mixture was poured into water (5 mL). After 5 min, the aqueous phase was extracted with ethyl acetate (2×10 mL). The combined organic layers were washed with saturated aqueous NH₄Cl (2×10 mL), dried over anhydrous Na₂SO₄, filtered, and concentrated under reduced pressure. Purification by prep-TLC gave N-((5-(1-hydroxycyclopropyl)-6-(isoxazol-3-ylmethoxy)-1-(phenylsulfonyl)-1H-indol-2-yl)methyl)-1-methylcyclopropane-1-carboxamide. LC-MS (ESI) m/z calcd for C₂₇H₂₇N₃O₆S: 521.16; found: 544.2 [M+Na]⁺.

### Step 8: N-((5-(1-fluorocyclopropyl)-6-(isoxazol-3-ylmethoxy)-1-(phenylsulfonyl)-1H-indol-2-yl)methyl)-1-methylcyclopropane-1-carboxamide

To a solution of N-((5-(1-hydroxycyclopropyl)-6-(isoxazol-3-ylmethoxy)-1-(phenylsulfonyl)-1H-indol-2-yl)methyl)-1-methylcyclopropane-1-carboxamide (300 mg, 575 μmol, 1.00 eq) in DCM (3 mL) was added N,N-diethyl-1,1,1-trifluoro-l⁴-sulfinimine (DAST, 111 mg, 690 mol, 1.20 eq) dropwise at −65° C. The reaction was allowed to proceed at −65° C. After 1 h, the reaction was poured into water (3 mL) and stirred for 2 min. The reaction mixture was extracted with ethyl acetate (2×5 mL). The combined organic layers were washed with brine (2×5 mL), dried over anhydrous Na₂SO₄, filtered, and concentrated under reduced pressure. Purification by prep-TLC gave N-((5-(1-fluorocyclopropyl)-6-(isoxazol-3-ylmethoxy)-1-(phenylsulfonyl)-1H-indol-2-yl)methyl)-1-methylcyclopropane-1-carboxamide. LC-MS (ESI) m/z calcd for C₂₇H₂₆FN₃O₅S: 523.16; found: 524.2 [M+H]⁺.

### Step 9: N-((5-(1-fluorocyclopropyl)-6-(isoxazol-3-ylmethoxy)-1H-indol-2-yl)methyl)-1-methylcyclopropane-1-carboxamide

To a solution of N-((5-(1-fluorocyclopropyl)-6-(isoxazol-3-ylmethoxy)-1-(phenylsulfonyl)-1H-indol-2-yl)methyl)-1-methylcyclopropane-1-carboxamide (119 mg, 227 μmol, 1.00 eq) in THF (0.2 mL) was added TBAF (1 M in THF, 1.14 mL, 5.00 eq) dropwise. After 16 h, the reaction was poured into water (2 mL) at 0° C. After 2 min, the reaction mixture was extracted with ethyl acetate (2×5 mL). The combined organic layers were washed with saturated aqueous NH₄Cl (2×5 mL), dried over anhydrous Na₂SO₄, filtered, and concentrated under reduced pressure. Purification by prep-HPLC gave N-((5-(1-fluorocyclopropyl)-6-(isoxazol-3-ylmethoxy)-1H-indol-2-yl)methyl)-1-methylcyclopropane-1-carboxamide (Compound 655). LC-MS (ESI) m/z calcd for C₂₁H₂₂FN₃O₃: 383.16; found: 382.0 [M−H]⁺. ¹H NMR (400 MHz, DMSO-d₆) δ 10.75 (s, 1H), 8.93 (s, 1H), 7.99 (t, J=5.6 Hz, 1H), 7.51 (d, J=2.0 Hz, 1H), 7.07 (s, 1H), 6.64 (s, 1H), 6.16 (s, 1H), 5.28 (s, 2H), 4.35 (d, J=5.6 Hz, 2H), 1.30-1.24 (m, 5H), 0.99-0.97 (m, 4H), 0.54-0.51 (m, 2H).

The following compounds in Table T-51 were synthesized using procedures similar to Compound 655 using the appropriate starting materials.

TABLE T-51

| | | | Exact | LCMS, |
|---|---|---|---|---|
| Compound | | | mass | found |
| No. | Structure | IUPAC name | (g/mol) | [M + H]⁺ |
| 656 | 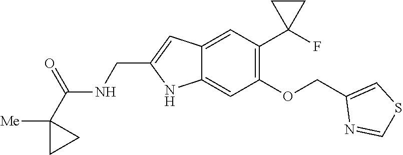 [SMILES: CC1(C(=O)NCc2cc3cc(C4(F)CC4)c(OCc4cscn4)cc3[nH]2)CC1] | N-((5-(1- fluorocyclopropyl)-6- (thiazol-4-ylmethoxy)- 1H-indol-2-yl)methyl)- 1-methylcyclopropane- 1-carboxamide | 399.14 | 397.7 [M − H]⁻ |

### Example 86 (Compound 657)

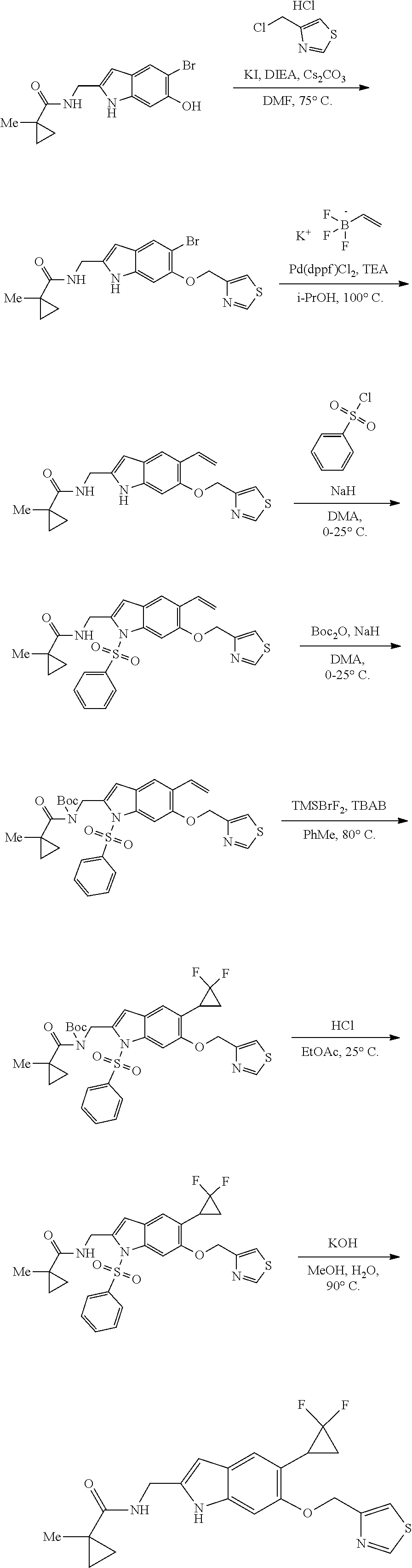

### Step 1: N-((5-bromo-6-(thiazol-4-ylmethoxy)-1H-indol-2-yl)methyl)-1-methylcyclopropane-1-carboxamide

To a solution of N-((5-bromo-6-hydroxy-1H-indol-2-yl)methyl)-1-methylcyclopropane-1-carboxamide (1.70 g, 5.26 mmol, 1.00 eq) in DMF (20 mL), was added 4-(chloromethyl)thiazole hydrochloride (984 mg, 5.79 mmol, 1.10 eq), KI (873 mg, 5.26 mmol, 1.00 eq), Cs₂CO₃ (3.43 g, 10.5 mmol, 2.00 eq), and DIEA (1.36 g, 10.5 mmol, 2.00 eq) in portions. The reaction mixture was degassed and purged with N₂ three times, then heated to 75° C. After 2 h, the reaction mixture was diluted with H₂O (50 mL) at 25° C. The reaction mixture was extracted with ethyl acetate (3×20 mL). The combined organic layers were washed with brine (20 mL), dried over anhydrous Na₂SO₄, filtered, and concentrated under reduced pressure. Purification by column chromatography gave N-((5-bromo-6-(thiazol-4-ylmethoxy)-1H-indol-2-yl)methyl)-1-methylcyclopropane-1-carboxamide. LC-MS (ESI) m/z calcd for C₁₈H₁₈BrN₃O₂S: 419.03; found: 420.2 [M+H]⁺.

### Step 2: 1-methyl-N-((6-(thiazol-4-ylmethoxy)-5-vinyl-1H-indol-2-yl)methyl)cyclopropane-1-carboxamide

To a solution of N-((5-bromo-6-(thiazol-4-ylmethoxy)-1H-indol-2-yl)methyl)-1-methylcyclopropane-1-carboxamide (1.10 g, 2.62 mmol, 1.00 eq) in i-PrOH (30 mL) was added potassium vinyltrifluoroborate (525 mg, 3.93 mmol, 1.50 eq), TEA (794 mg, 7.85 mmol, 3.00 eq), and Pd(dppf)Cl₂·CH₂Cl₂ (213 mg, 261 μmol, 0.10 eq). The reaction mixture was degassed and purged with N₂ three times, and then it was heated to 100° C. After 4 h, the reaction mixture was diluted with H₂O (50 mL) at 25° C. The reaction mixture was extracted with ethyl acetate (3×20 mL). The combined organic layers were washed with brine (20 mL), dried over anhydrous Na₂SO₄, filtered, and concentrated under reduced pressure. Purification by prep-TLC gave 1-methyl-N-((6-(thiazol-4-ylmethoxy)-5-vinyl-1H-indol-2-yl)methyl)cyclopropane-1-carboxamide. LC-MS (ESI) m/z calcd for C₂₀H₂₁N₃O₂S: 367.14; found: 368.2 [M+H]⁺.

### Step 3: 1-methyl-N-((1-(phenylsulfonyl)-6-(thiazol-4-ylmethoxy)-5-vinyl-1H-indol-2-yl)methyl)cyclopropane-1-carboxamide

To a solution of 1-methyl-N-((6-(thiazol-4-ylmethoxy)-5-vinyl-1H-indol-2-yl)methyl)cyclopropane-1-carboxamide (400 mg, 1.09 mmol, 1.00 eq) in DMA (7 mL) at 0° C. was added NaH (130 mg, 3.27 mmol, 3.00 eq) in portions. After 30 min at 0° C., a solution of benzenesulfonyl chloride (384 mg, 2.18 mmol, 2.00 eq) in N,N-dimethylacetamide (DMA, 7 mL) added dropwise. The reaction mixture was allowed to warm to 25° C. After 1 h the reaction mixture was quenched with saturated aqueous NH₄Cl (50 mL) at 0° C. The reaction mixture was extracted with ethyl acetate (3×20 mL). The combined organic layers were washed with brine (20 mL), dried over anhydrous Na₂SO₄, filtered, and concentrated under reduced pressure. Purification by column chromatography gave 1-methyl-N-((1-(phenylsulfonyl)-6-(thiazol-4-ylmethoxy)-5-vinyl-1H-indol-2-yl)methyl)cyclopropane-1-carboxamide.

### Step 4: tert-butyl (1-methylcyclopropane-1-carbonyl)((1-(phenylsulfonyl)-6-(thiazol-4-ylmethoxy)-5-vinyl-1H-indol-2-yl)methyl)carbamate

To a solution of 1-methyl-N-((1-(phenylsulfonyl)-6-(thiazol-4-ylmethoxy)-5-vinyl-1H-indol-2-yl)methyl)cyclopropane-1-carboxamide (450 mg, 886 μmol, 1.00 eq) in DMA (6 mL) at 0° C. was added NaH (106 mg, 2.66 mmol, 60% purity, 3.00 eq) in portions. After 30 min at 0° C., a solution of Boc₂O (967 mg, 4.43 mmol, 1.02 mL, 5.00 eq) in DMA (6 mL) was added dropwise. The reaction mixture was allowed to warmed to 25° C. After 1 h, the reaction mixture was quenched with saturated aqueous NH₄Cl (50 mL) at 0° C. The reaction mixture was extracted with ethyl acetate (3×20 mL). The combined organic layers were washed with brine (20 mL), dried over anhydrous Na₂SO₄, filtered, and concentrated under reduced pressure. Purification by column chromatography gave tert-butyl (1-methylcyclopropane-1-carbonyl)((1-(phenylsulfonyl)-6-(thiazol-4-ylmethoxy)-5-vinyl-1H-indol-2-yl)methyl)carbamate.

### Step 5: tert-butyl ((5-(2,2-difluorocyclopropyl)-1-(phenylsulfonyl)-6-(thiazol-4-ylmethoxy)-1H-indol-2-yl)methyl)(1-methylcyclopropane-1-carbonyl)carbamate

To a solution of tert-butyl (1-methylcyclopropane-1-carbonyl)((1-(phenylsulfonyl)-6-(thiazol-4-ylmethoxy)-5-vinyl-1H-indol-2-yl)methyl)carbamate (230 mg, 378 μmol, 1.00 eq) in toluene (3 mL) was added TBAB (61.0 mg, 189 μmol, 0.50 eq) dropwise followed by [bromo(difluoro)methyl]-trimethyl-silane (1.54 g, 7.57 mmol, 20.0 eq) in one portion. The reaction mixture was then heated to 80° C. After 12 h, the reaction mixture was diluted with saturated aqueous NH₄Cl (50 mL) at 25° C. The reaction solution was extracted with ethyl acetate (3×20 mL). The combined organic layers were washed with brine (20 mL), dried over anhydrous Na₂SO₄, filtered, and concentrated under reduced pressure. Purification by prep-TLC gave tert-butyl ((5-(2,2-difluorocyclopropyl)-1-(phenylsulfonyl)-6-(thiazol-4-ylmethoxy)-1H-indol-2-yl)methyl)(1-methylcyclopropane-1-carbonyl)carbamate. LC-MS (ESI) m/z calcd for C₃₂H₃₃F₂N₃O₆S₂: 657.18; found: 558.1 [M−Boc+H]⁺.

### Step 6: N-((5-(2,2-difluorocyclopropyl)-1-(phenylsulfonyl)-6-(thiazol-4-ylmethoxy)-1H-indol-2-yl)methyl)-1-methylcyclopropane-1-carboxamide hydrochloride

To a solution of tert-butyl ((5-(2,2-difluorocyclopropyl)-1-(phenylsulfonyl)-6-(thiazol-4-ylmethoxy)-1H-indol-2-yl)methyl)(1-methylcyclopropane-1-carbonyl)carbamate (85.0 mg, 129 μmol, 1.00 eq) in ethyl acetate (2 mL) was added HCl (4 M in EtOAc, 5 mL, 154 eq) dropwise. After 1 h, the reaction mixture was concentrated under reduced pressure to give N-((5-(2,2-difluorocyclopropyl)-1-(phenylsulfonyl)-6-(thiazol-4-ylmethoxy)-1H-indol-2-yl)methyl)-1-methylcyclopropane-1-carboxamide, which was used directly without further purification.

### Step 7: N-((5-(2,2-difluorocyclopropyl)-6-(thiazol-4-ylmethoxy)-1H-indol-2-yl)methyl)-1-methylcyclopropane-1-carboxamide

To a solution of N-((5-(2,2-difluorocyclopropyl)-1-(phenylsulfonyl)-6-(thiazol-4-ylmethoxy)-1H-indol-2-yl)methyl)-1-methylcyclopropane-1-carboxamide (82.0 mg, 147 μmol, 1.00 eq) in MeOH (6 mL) and H₂O (2 mL) at 25° C. was added KOH (41.2 mg, 735 μmol, 5.00 eq). The reaction mixture was heated to 90° C. After 12 h, the reaction mixture was diluted with H₂O (20 mL) at 25° C. The reaction mixture was extracted with ethyl acetate (3×20 mL). The combined organic layers were washed with brine (20 mL), dried over anhydrous Na₂SO₄, filtered, and concentrated under reduced. Purification by prep-HPLC gave N-((5-(2,2-difluorocyclopropyl)-6-(thiazol-4-ylmethoxy)-1H-indol-2-yl)methyl)-1-methylcyclopropane-1-carboxamide (Compound 657). LC-MS (ESI) m/z calcd for C₂₁H₂₁F₂N₃O₂S: 417.13; found: 418.0 [M+H]⁺. ¹H NMR (400 MHz, DMSO-d₆) δ 10.61 (s, 1H), 9.14 (d, J=2.0 Hz, 1H), 7.97 (t, J=6.0 Hz, 1H), 7.68 (d, J=2.0 Hz, 1H), 7.21 (s, 1H), 7.06 (s, 1H), 6.10 (s, 1H), 5.26 (s, 2H), 4.34 (d, J=5.6 Hz, 2H), 3.00-2.94 (m, 1H), 1.89-1.81 (m, 2H), 1.28 (s, 3H), 1.00-0.97 (m, 2H), 0.53-0.50 (m, 2H).

The following compounds in Table T-52 were synthesized using procedures similar to Compound 657 using the appropriate starting materials.

TABLE T-52

| | | | Exact | LCMS, |
|---|---|---|---|---|
| Compound | | | mass | found |
| No. | Structure | IUPAC name | (g/mol) | [M + H]⁺ |
| 658 | 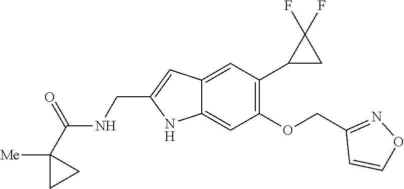 [SMILES: CC1(C(=O)NCc2cc3cc(C4CC4(F)F)c(OCc4ccon4)cc3[nH]2)CC1] | N-((5-(2,2- difluorocyclopropyl)-6- (isoxazol-3-ylmethoxy)- 1H-indol-2-yl)methyl)- 1-methylcyclopropane- 1-carboxamide | 401.16 | 402.0 |

### Example 87 (Compound 659)

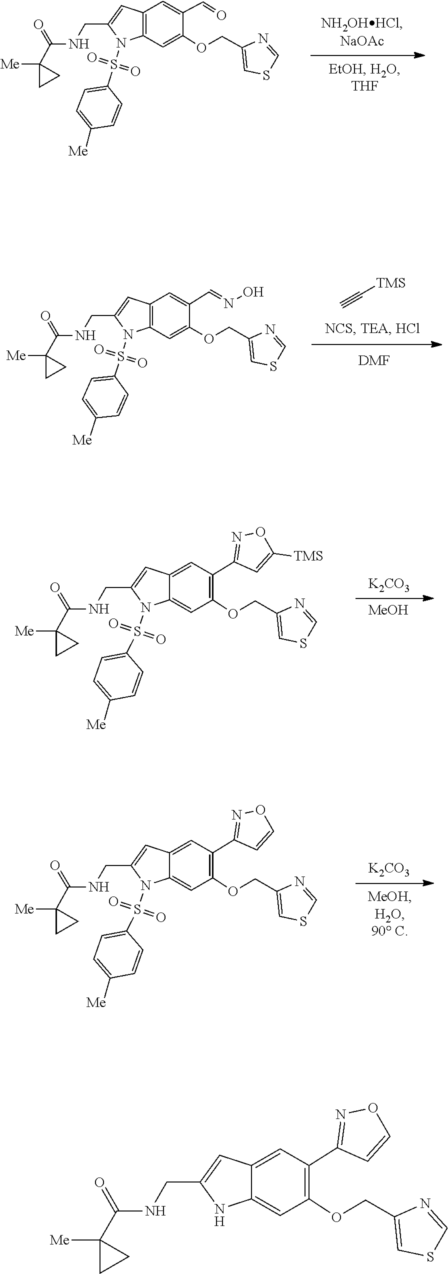

### Step 1: (E)-N-((5-((hydroxyimino)methyl)-6-(thiazol-4-ylmethoxy)-1-tosyl-1H-indol-2-yl)methyl)-1-methylcyclopropane-1-carboxamide

To a solution of N-((5-formyl-6-(thiazol-4-ylmethoxy)-1-tosyl-1H-indol-2-yl)methyl)-1-methylcyclopropane-1-carboxamide (1.00 g, 1.91 mmol, 1.00 eq) in EtOH (5 mL), water (5 mL), and THF (15 mL) were added sodium acetate (313 mg, 3.82 mmol, 2.00 eq) and NH₂OH·HCl (199 mg, 2.86 mmol, 1.50 eq). The reaction mixture was stirred at 25° C. for 16 h. The reaction mixture was quenched with water (30 mL) and adjusted to pH=7 with addition of aqueous HCl (1 M). The resulting mixture was extracted with EtOAc (3×20 mL). The combined organic layers were washed with brine (20 mL), dried over anhydrous Na₂SO₄, filtered, and concentrated under reduced pressure to give (E)-N-((5-((hydroxyimino)methyl)-6-(thiazol-4-ylmethoxy)-1-tosyl-1H-indol-2-yl)methyl)-1-methylcyclopropane-1-carboxamide. LC-MS (ESI) m/z calcd for C₂₆H₂₆N₄O₅S: 538.13; found: 539.2 [M+H]⁺.

### Step 2: 1-methyl-N-((6-(thiazol-4-ylmethoxy)-1-tosyl-5-(5-(trimethylsilyl)isoxazol-3-yl)-1H-indol-2-yl)methyl)cyclopropane-1-carboxamide

To a solution of (E)-N-((5-((hydroxyimino)methyl)-6-(thiazol-4-ylmethoxy)-1-tosyl-1H-indol-2-yl)methyl)-1-methylcyclopropane-1-carboxamide (820 mg, 1.52 mmol, 1.00 eq) in DMF (3 mL) were added aqueous HCl (1 M, 152 μL, 152 μmol, 0.1 eq) and NCS (244 mg, 1.83 mmol, 1.20 eq). The reaction mixture was stirred at 25° C. for 1 h, and ethynyltrimethylsilane (254 mg, 2.59 mmol, 1.70 eq) and TEA (154 mg, 1.52 mmol, 1.00 eq) were added. The reaction mixture was stirred for an additional 16 h at 25° C. The reaction mixture was then quenched with the addition of water (15 mL) and the mixture was extracted with EtOAc (3×10 mL). The combined organic layers were washed with brine (10 mL), dried over anhydrous Na₂SO₄, filtered, and concentrated under reduced pressure. Purification by column chromatography gave 1-methyl-N-((6-(thiazol-4-ylmethoxy)-1-tosyl-5-(5-(trimethylsilyl)isoxazol-3-yl)-1H-indol-2-yl)methyl)cyclopropane-1-carboxamide. LC-MS (ESI) m/z calcd for C₃₁H₃₄N₄O₅S2Si: 634.17; found: 635.3 [M+H]⁺.

### Step 3: N-((5-(isoxazol-3-yl)-6-(thiazol-4-ylmethoxy)-1-tosyl-1H-indol-2-yl)methyl)-1-methylcyclopropane-1-carboxamide

To a solution of a solution of 1-methyl-N-((6-(thiazol-4-ylmethoxy)-1-tosyl-5-(5-(trimethylsilyl)isoxazol-3-yl)-1H-indol-2-yl)methyl)cyclopropane-1-carboxamide (120 mg, 183 μmol, 1.00 eq) in MeOH (2 mL) was added K₂CO₃ (51.0 mg, 365 μmol, 2.00 eq), and the reaction was stirred at 25° C. for 1 h. The reaction mixture was then quenched with the addition of water (5 mL), and the resulting mixture as extracted with EtOAc (3×10 mL). The combined organic layers were washed with brine (10 mL), dried over anhydrous Na₂SO₄, filtered, and concentrated under reduced pressure to give N-((5-(isoxazol-3-yl)-6-(thiazol-4-ylmethoxy)-1-tosyl-1H-indol-2-yl)methyl)-1-methylcyclopropane-1-carboxamide. LC-MS (ESI) m/z calcd for C₂₈H₂₆N₄O₅S₂: 562.13; found: 563.2 [M+H]⁺.

### Step 4: N-((5-(isoxazol-3-yl)-6-(thiazol-4-ylmethoxy)-1H-indol-2-yl)methyl)-1-methylcyclopropane-1-carboxamide

To a solution of a solution of N-((5-(isoxazol-3-yl)-6-(thiazol-4-ylmethoxy)-1-tosyl-1H-indol-2-yl)methyl)-1-methylcyclopropane-1-carboxamide (100 mg, 178 μmol, 1.00 eq) in MeOH (0.9 mL) and H₂O (0.3 mL) was added K₂CO₃ (123 mg, 888 μmol, 5.00 eq), and the reaction was heated to 90° C. for 1 h. The reaction mixture was cooled to 25° C. and quenched with the addition of water (5 mL). The resulting mixture was extracted with EtOAc (3×10 mL). The combined organic layers were washed with brine (10 mL), dried over anhydrous Na₂SO₄, filtered, and concentrated under reduced pressure to give N-((5-(isoxazol-3-yl)-6-(thiazol-4-ylmethoxy)-1H-indol-2-yl)methyl)-1-methylcyclopropane-1-carboxamide (Compound 659). LC-MS (ESI) m/z calcd for C₂₁H₂₀N₄O₃S: 408.13; found: 409.3 [M+H]⁺. ¹H NMR (400 MHz, DMSO-d₆) δ 10.80 (s, 1H), 9.16 (s, 1H), 8.84 (s, 1H), 8.04 (t, J=6.0 Hz, 1H), 7.84 (s, 1H), 7.77 (d, J=1.2 Hz, 1H), 7.22 (s, 1H), 6.94 (d, J=2.0 Hz, 1H), 6.22 (s, 1H), 5.30 (s, 2H), 4.38 (d, J=5.6 Hz, 2H), 1.30 (s, 3H), 1.02-0.99 (m, 2H), 0.55-0.52 (m, 2H).

### Example 88 (Compound 660)

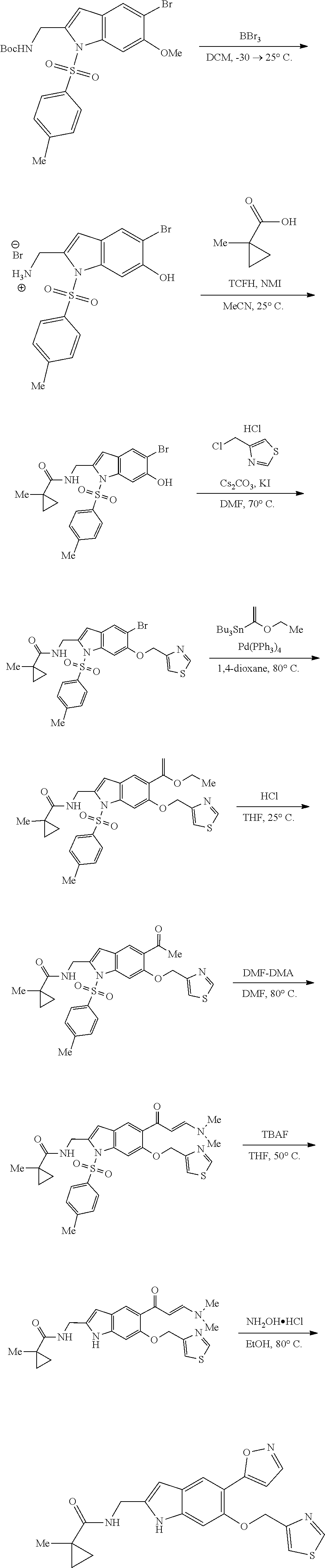

### Step 1: 2-(aminomethyl)-5-bromo-1-tosyl-1H-indol-6-ol hydrobromide

A solution of tert-butyl N-[[5-bromo-6-methoxy-1-(p-tolylsulfonyl)indol-2-yl]methyl]carbamate (4.00 g, 7.85 mmol, 1.00 eq) in DCM (30 mL) was cooled to −30° C. and degassed and purged with N₂ three times, and BBr₃ (23.6 mmol, 2.27 mL, 3.00 eq) was added dropwise to the reaction mixture. After the addition, the reaction mixture was warmed to 25° C. and stirred for 2 h. The reaction mixture was cooled to 0° C., and MeOH (50 mL) was added dropwise. The mixture was allowed to warm to room temperature and stirred for 30 min. The reaction mixture was concentrated under reduced pressure. Methyl tert-butyl ether/Ethyl acetate=2/1 (30 mL) was added to the residue and gently agitated for 20 min and then filtered. Collection of the filter cake gave 2-(aminomethyl)-5-bromo-1-tosyl-1H-indol-6-ol hydrobromide. LC-MS (ESI) m/z calcd for C₁₆H₁₅BrN₂O₃S: 394.00; found: 378.0 [M−NH₂]⁺.

### Step 2: N-((5-bromo-6-hydroxy-1-tosyl-1H-indol-2-yl)methyl)-1-methylcyclopropane-1-carboxamide

To a mixture of 2-(aminomethyl)-5-bromo-1-tosyl-1H-indol-6-ol hydrobromide (3.50 g, 6.20 mmol, 70% purity, 1.00 eq) and 1-methylcyclopropane-1-carboxylic acid (1.24 g, 12.4 mmol, 2.00 eq) in MeCN (30 mL) cooled to 0° C. was added NMI (2.54 g, 31.0 mmol, 2.47 mL, 5.00 eq). TCFH (2.09 g, 7.44 mmol, 1.20 eq) was then added portionwise to the reaction mixture. After the addition, the reaction mixture was warmed to 25° C. and stirred for 1 h. The reaction mixture was diluted with H₂O (120 mL) and extracted with ethyl acetate (3×40 mL). The combined organic phases were washed with brine (40 mL), dried over Na₂SO₄, filtered, and concentrated under reduced pressure. Purification by column chromatography gave N-((5-bromo-6-hydroxy-1-tosyl-1H-indol-2-yl)methyl)-1-methylcyclopropane-1-carboxamide. LC-MS (ESI) m/z calcd for C₂₁H₂₁BrN₂O₄S: 476.04; found: 477.1 [M+H]⁺.

### Step 3: N-((5-bromo-6-(thiazol-4-ylmethoxy)-1-tosyl-1H-indol-2-yl)methyl)-1-methylcyclopropane-1-carboxamide

To a mixture of N-((5-bromo-6-hydroxy-1-tosyl-1H-indol-2-yl)methyl)-1-methylcyclopropane-1-carboxamide (3.00 g, 6.28 mmol, 1.00 eq) and 4-(chloromethyl)thiazole (1.28 g, 7.54 mmol, 1.20 eq, HCl salt) in DMF (40 mL) was added KI (1.04 g, 6.28 mmol, 1.00 eq) and Cs₂CO₃ (6.14 g, 18.8 mmol, 3.00 eq) in one portion at 25° C. The reaction mixture was heated to 70° C. and stirred for 6 h. The reaction mixture was allowed to cool to room temperature and was quenched by addition of H₂O (150 mL). The mixture was extracted with ethyl acetate (3×50 mL). The combined organic phases were washed with brine (50 mL), dried over Na₂SO₄, filtered, and concentrated under reduced pressure. Purification by column chromatography gave N-((5-bromo-6-(thiazol-4-ylmethoxy)-1-tosyl-1H-indol-2-yl)methyl)-1-methylcyclopropane-1-carboxamide. LC-MS (ESI) m/z calcd for C₂₅H₂₄BrN₃O₄S₂: 573.04; found: 574.2 [M+H]⁺.

### Step 4: N-((5-(1-ethoxyvinyl)-6-(thiazol-4-ylmethoxy)-1-tosyl-1H-indol-2-yl)methyl)-1-methylcyclopropane-1-carboxamide

To a solution of N-((5-bromo-6-(thiazol-4-ylmethoxy)-1-tosyl-1H-indol-2-yl)methyl)-1-methylcyclopropane-1-carboxamide (2.10 g, 3.66 mmol, 1.00 eq) in 1,4-dioxane (30 mL) was added tributyl(1-ethoxyvinyl)stannane (2.24 g, 6.20 mmol, 2.09 mL, 1.70 eq) at 25° C. Pd(PPh₃)₄ (422 mg, 365 μmol, 0.10 eq) was added in one portion, and the reaction mixture was degassed and purged with N₂ three times. The reaction mixture was warmed to 80° C. and stirred for 16 h. The reaction mixture was cooled to room temperature and quenched by addition of saturated aqueous KF solution (100 mL) and stirred for 0.5 h. The mixture was filtered, and the filtrate was extracted with ethyl acetate (3×30 mL). The combined organic phases were washed with brine (30 mL), dried over Na₂SO₄, and filtered. Concentration under reduced pressure gave N-((5-(1-ethoxyvinyl)-6-(thiazol-4-ylmethoxy)-1-tosyl-1H-indol-2-yl)methyl)-1-methylcyclopropane-1-carboxamide, which was used without further purification. LC-MS (ESI) m/z calcd for C₂₉H₃₁N₃O₅S₂: 565.17; found: 566.3 [M+H]⁺.

### Step 5: N-((5-acetyl-6-(thiazol-4-ylmethoxy)-1-tosyl-1H-indol-2-yl)methyl)-1-methylcyclopropane-1-carboxamide

To a solution of N-((5-(1-ethoxyvinyl)-6-(thiazol-4-ylmethoxy)-1-tosyl-1H-indol-2-yl)methyl)-1-methylcyclopropane-1-carboxamide (3.50 g, 3.09 mmol, 50% purity, 1.00 eq) in THF (40 mL) was added HCl (3 M in H₂O, 20 mL, 19.4 eq) in one portion at 25° C. The reaction was stirred at 25° C. for 2 h. The reaction mixture was quenched by addition of H₂O (60 mL) and extracted with ethyl acetate (3×20 mL). The combined organic phases were washed with saturated aqueous NaHCO₃ solution (20 mL), dried over Na₂SO₄, filtered, and concentrated under reduced pressure. Purification by column chromatography was followed by further purification by filtration with ethyl acetate (15 mL). The filter cake was collected to give N-((5-acetyl-6-(thiazol-4-ylmethoxy)-1-tosyl-1H-indol-2-yl)methyl)-1-methylcyclopropane-1-carboxamide. LC-MS (ESI) m/z calcd for C₂₇H₂₇N₃O₅S₂: 537.14; found: 538.2 [M+H]⁺.

### Step 6: (E)-N-((5-(3-(dimethylamino)acryloyl)-6-(thiazol-4-ylmethoxy)-1-tosyl-1H-indol-2-yl)methyl)-1-methylcyclopropane-1-carboxamide

To a solution of N-((5-acetyl-6-(thiazol-4-ylmethoxy)-1-tosyl-1H-indol-2-yl) methyl)-1-methylcyclopropane-1-carboxamide (400 mg, 744 μmol, 1.00 eq) in DMF (10 mL) was added 1,1-dimethoxy-N,N-dimethylmethanamine (443 mg, 3.72 mmol, 5.00 eq) dropwise at 25° C. under an atmosphere of N₂. The reaction mixture was heated to 80° C. and stirred for 16 h. The reaction mixture was allowed to cool to room temperature and quenched by addition of H₂O (60 mL). The mixture was extracted with ethyl acetate (3×20 mL). The combined organic phase was washed with brine (20 mL), dried over Na₂SO₄, filtered, and concentrated under reduced pressure. The residue was triturated with methyl tert-butyl ether (2×15 mL) and filtered. Collection of the filter cake gave (E)-N-((5-(3-(dimethylamino)acryloyl)-6-(thiazol-4-ylmethoxy)-1-tosyl-1H-indol-2-yl)methyl)-1-methylcyclopropane-1-carboxamide. LC-MS (ESI) m/z calcd for C₃₀H₃₂N₄O₅S₂: 592.18; found: 593.3 [M+H]⁺.

### Step 7: N-[[5-[(E)-3-(dimethylamino)prop-2-enoyl]-6-(thiazol-4-ylmethoxy)-1H-indol-2-yl]methyl]-1-methyl-cyclopropanecarboxamide

To a solution of (E)-N-((5-(3-(dimethylamino)acryloyl)-6-(thiazol-4-ylmethoxy)-1-tosyl-1H-indol-2-yl)methyl)-1-methylcyclopropane-1-carboxamide (290 mg, 489 μmol, 1.00 eq) in THF (2 mL) was added TBAF (1 M in THF, 2.5 mL, 5.00 eq) dropwise at 25° C. The reaction mixture was heated to 50° C. and stirred for 4 h. The reaction mixture was allowed to cool to room temperature and quenched by addition of H₂O (60 mL). The mixture was extracted with ethyl acetate (2×20 mL). The combined organic phases were washed with saturated aqueous NH₄Cl solution (20 mL), dried over Na₂SO₄, filtered, and concentrated under reduced pressure. The residue was triturated with methyl tert-butyl ether (10 mL) and then filtered. Collection of the filter cake gave N-[[5-[(E)-3-(dimethylamino)prop-2-enoyl]-6-(thiazol-4-ylmethoxy)-1H-indol-2-yl]methyl]-1-methyl-cyclopropanecarboxamide. LC-MS (ESI) m/z calcd for C₂₃H₂₆N₄O₃S: 438.17; found: 439.2 [M+H]⁺.

### Step 8: N-((5-(isoxazol-5-yl)-6-(thiazol-4-ylmethoxy)-1H-indol-2-yl)methyl)-1-methylcyclopropane-1-carboxamide

To a solution of N-[[5-[(E)-3-(dimethylamino)prop-2-enoyl]-6-(thiazol-4-ylmethoxy)-1H-indol-2-yl]methyl]-1-methyl-cyclopropanecarboxamide (160 mg, 365 μmol, 1.00 eq) in EtOH (3 mL) was added NH₂OH·HCl (76.0 mg, 1.09 mmol, 3.00 eq) at 25° C. The reaction mixture was heated to 80° C. and stirred for 2 h. The reaction mixture was cooled to 0° C. and quenched by addition of H₂O (30 mL). It was then extracted with ethyl acetate (3×10 mL). The combined organic phases were washed with brine (10 mL), dried over Na₂SO₄, filtered, and concentrated under reduced pressure. Purification by column chromatography and prep-HPLC gave N-((5-(isoxazol-5-yl)-6-(thiazol-4-ylmethoxy)-1H-indol-2-yl)methyl)-1-methylcyclopropane-1-carboxamide (Compound 660). LC-MS (ESI) m/z calcd for C₂₁H₂₀N₄O₃S: 408.13; found: 409.3 [M+H]⁺. ¹H NMR (400 MHz, DMSO-d₆) δ 10.89 (s, 1H), 9.19 (d, J=2.0 Hz, 1H), 8.53 (d, J=1.6 Hz, 1H), 8.05 (t, J=6.0 Hz, 1H), 7.98 (s, 1H), 7.83 (s, 1H), 7.24 (s, 1H), 6.72 (s, 1H), 6.27 (s, 1H), 5.35 (s, 2H), 4.38 (d, J=5.6 Hz, 2H), 1.30 (s, 3H), 1.02-1.00 (m, 2H), 0.55-0.53 (m, 2H).

### Example 89 (Compound 661)

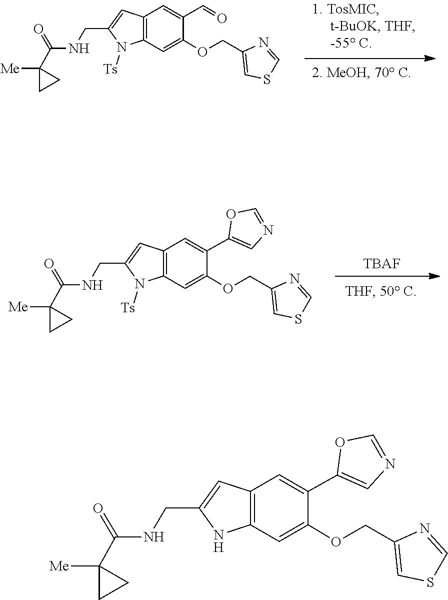 [SMILES: Cc1ccc(S(=O)(=O)n2c(CNC(=O)C3(C)CC3)cc3cc(C=O)c(OCc4cscn4)cc32)cc1 ; Cc1ccc(S(=O)(=O)n2c(CNC(=O)C3(C)CC3)cc3cc(-c4cnco4)c(OCc4cscn4)cc32)cc1 ; "TBAF" ; CC1(C(=O)NCc2cc3cc(-c4cnco4)c(OCc4cscn4)cc3[nH]2)CC1]

### Step 1: 1-methyl-N-((5-(oxazol-5-yl)-6-(thiazol-4-ylmethoxy)-1-tosyl-1H-indol-2-yl)methyl)cyclopropane-1-carboxamide

To a solution of t-BuOK (1 M, 840 μL, 2.00 eq) in THF (2 mL) at −55° C. was added a solution of 1-(isocyanomethylsulfonyl)-4-methyl-benzene (90.0 mg, 462 μmol, 1.10 eq) in THF (2 mL) dropwise. The mixture was stirred at −55° C. for 15 min. A solution of N-((5-formyl-6-(thiazol-4-ylmethoxy)-1-tosyl-1H-indol-2-yl)methyl)-1-methylcyclopropane-1-carboxamide (220 mg, 420 μmol, 1.00 eq) in THF (2 mL) was added at dropwise at −55° C. The reaction mixture was then stirred at −55° C. for 2 h. The reaction mixture was then warmed to 25° C., and MeOH (5 mL) was added dropwise. The reaction mixture was then heated to 70° C. for 0.5 h. After cooling to room temperature, the reaction mixture was concentrated in vacuo. The resulting residue was diluted with H₂O (10 mL) and extracted with EtOAc (3×20 mL). The combined organic phases were washed with brine (2×20 mL), dried over anhydrous Na₂SO₄, filtered, and concentrated under reduced pressure. Purification by column chromatography gave 1-methyl-N-((5-(oxazol-5-yl)-6-(thiazol-4-ylmethoxy)-1-tosyl-1H-indol-2-yl)methyl)cyclopropane-1-carboxamide. LC-MS (ESI) m/z calcd for C₂₈H₂₆N₄O₅S₂: 562.13; found: 563.4 [M+H]⁺.

### Step 2: 1-methyl-N-((5-(oxazol-5-yl)-6-(thiazol-4-ylmethoxy)-1H-indol-2-yl)methyl)cyclopropane-1-carboxamide

To a solution of 1-methyl-N-((5-(oxazol-5-yl)-6-(thiazol-4-ylmethoxy)-1-tosyl-1H-indol-2-yl)methyl)cyclopropane-1-carboxamide (100 mg, 177 μmol, 1.00 eq) in THF (0.5 mL) was added TBAF (1 M in THF, 535 μL, 3.00 eq) dropwise, and the reaction mixture was heated and stirred at 50° C. for 3 h. After cooling to room temperature, the reaction mixture was concentrated under reduced pressure. The resulting residue was diluted with H₂O (10 mL) and extracted with ethyl acetate (3×20 mL). The combined organic phases were washed with brine (2×20 mL), dried over anhydrous Na₂SO₄, filtered, and concentrated under reduced pressure. Purification by prep-HPLC gave 1-methyl-N-((5-(oxazol-5-yl)-6-(thiazol-4-ylmethoxy)-1H-indol-2-yl)methyl)cyclopropane-1-carboxamide (Compound 661). LC-MS (ESI) m/z calcd for C₂₁H₂₀N₄O₃S: 408.13; found 409.1 [M+H]⁺. ¹H NMR (400 MHz, DMSO-d₆) δ 10.8 (s, 1H), 9.21 (d, J=7.6, 1H), 8.32 (s, 1H), 8.03 (t, J=5.6, 1H), 7.84 (s, 1H), 7.80 (s, 1H), 7.35 (s, 1H), 7.21 (s, 1H), 6.23 (s, 1H), 5.34 (s, 2H), 4.37 (d, J=5.6, 2H), 1.30 (s, 3H), 1.02-0.99 (m, 2H), 0.55-0.52 (m, 2H).

### Example 90 (Compound 662)

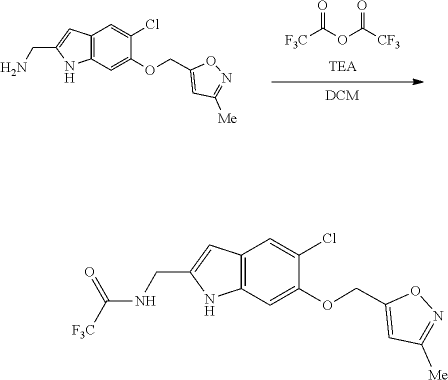 [SMILES: Cc1cc(COc2cc3[nH]c(CN)cc3cc2Cl)on1 ; O=C(OC(=O)C(F)(F)F)C(F)(F)F ; Cc1cc(COc2cc3[nH]c(CNC(=O)C(F)(F)F)cc3cc2Cl)on1]

### Step 1: N-((5-chloro-6-((3-methylisoxazol-5-yl)methoxy)-1H-indol-2-yl)methyl)-2,2,2-trifluoroacetamide

To a solution of (5-chloro-6-((3-methylisoxazol-5-yl)methoxy)-1H-indol-2-yl)methanamine (50 mg, 0.171 mmol, 1.0 eq.) in DCM (1.1 mL) was added triethylamine (0.072 mL, 0.514 mmol, 3 eq.) followed by trifluoroacetic anhydride (0.036 g, 0.171 mmol, 1.0 eq.). The resulting mixture was stirred for 1 h then water (1 mL) was added, and the mixture was concentrated under reduced pressure. The resulting solid was purified by prep-HPLC to afford N-((5-chloro-6-((3-methylisoxazol-5-yl)methoxy)-1H-indol-2-yl)methyl)-2,2,2-trifluoroacetamide (Compound 662). LC-MS (ESI) m/z calcd for C₁₆H₁₃ClF₃N₃O₃: 387.06; found 388.1. ¹H NMR (500 MHz, DMSO) δ 11.05 (d, J=2.1 Hz, 1H), 9.92 (s, 1H), 7.48 (s, 1H), 7.09 (s, 1H), 6.39 (s, 1H), 6.16 (d, J=2.0 Hz, 1H), 5.23 (s, 2H), 4.44 (d, J=5.0 Hz, 2H), 2.17 (s, 3H).

### Example 91 (Compound 663)

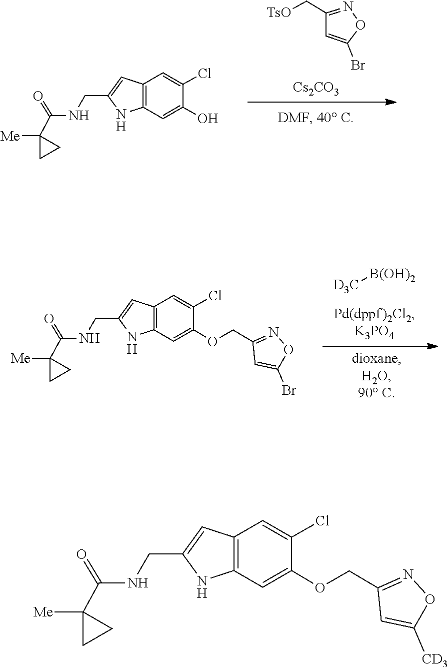 [SMILES: Cc1ccc(S(=O)(=O)OCc2cc(Br)on2)cc1 ; CC1(C(=O)NCc2cc3cc(Cl)c(O)cc3[nH]2)CC1 ; "Cs2CO3" ; [2H]C([2H])([2H])B(O)O ; CC1(C(=O)NCc2cc3cc(Cl)c(OCc4cc(Br)on4)cc3[nH]2)CC1 ; [2H]C([2H])([2H])c1cc(COc2cc3[nH]c(CNC(=O)C4(C)CC4)cc3cc2Cl)no1]

### Step 1: N-((6-((5-bromoisoxazol-3-yl)methoxy)-5-chloro-1H-indol-2-yl)methyl)-1-methylcyclopropane-1-carboxamide

N-((5-chloro-6-hydroxy-1H-indol-2-yl)methyl)-1-methylcyclopropane-1-carboxamide (75 mg, 0.269 mmol, 1 eq.), (5-bromoisoxazol-3-yl)methyl 4-methylbenzenesulfonate (0.096 g, 0.288 mmol, 1.07 eq.) and cesium carbonate (0.175 g, 0.538 mmol, 2 eq.) were dissolved in DMF (1.34 mL), and the resulting mixture was heated to 40° C. After 16 h, the reaction was cooled to 20° C. and diluted with sat. aq. NH₄Cl (30 mL). The aqueous layer was extracted with EtOAc (3×30 mL), and the combined organic phases were washed with sat. aq. NaCl (50 mL), dried over sodium sulfate, filtered, and concentrated under reduced pressure. The resulting residue was purified by column chromatography to give N-((6-((5-bromoisoxazol-3-yl)methoxy)-5-chloro-1H-indol-2-yl)methyl)-1-methylcyclopropane-1-carboxamide. LC-MS (ESI) m/z calcd for C₁₈H₁₇BrClN₃O₃: 437.01; found 438.1 [M+H]⁺.

### Step 2: N-((5-chloro-6-((5-(methyl-d3)isoxazol-3-yl)methoxy)-1H-indol-2-yl)methyl)-1-methylcyclopropane-1-carboxamide

A mixture of N-((6-((5-bromoisoxazol-3-yl)methoxy)-5-chloro-1H-indol-2-yl)methyl)-1-methylcyclopropane-1-carboxamide (0.074 g, 0.169 mmol, 1 eq.), methyl-D3-boronic acid (0.013 g, 0.202 mmol, 1.2 eq.), K₃PO₄ (0.072 g, 0.337 mmol, 2 eq.) was dissolved in dioxane (800 μL) and water (80 μL). The reaction mixture was sparged with N₂ for 10 min followed by addition of Pd(dppf)₂Cl₂·CH₂Cl₂ (0.014 g, 0.017 mmol, 0.1 eq.). The reaction mixture was further sparged with N₂ for 2 minutes and heated to 90° C. for 16 h. The reaction mixture was then cooled to 25° C., diluted with EtOAc (10 mL), filtered over Celite, and concentrated under reduced pressure. The resulting residue was purified by prep-HPLC to give N-((5-chloro-6-((5-(methyl-d3)isoxazol-3-yl)methoxy)-1H-indol-2-yl)methyl)-1-methylcyclopropane-1-carboxamide (Compound 663). LC-MS (ESI) m/z calcd for C₁₉H₁₇D3ClN₃O₃: 376.14; found 377.2 [M+H]⁺. ¹H NMR (500 MHz, DMSO) δ 10.76 (s, 1H), 7.95 (t, J=5.8 Hz, 1H), 7.43 (s, 1H), 7.10 (s, 1H), 6.27 (s, 1H), 6.05 (s, 1H), 5.13 (s, 2H), 4.28 (d, J=5.7 Hz, 2H), 1.22 (s, 3H), 0.93 (d, J=2.9 Hz, 2H), 0.47 (q, J=3.6 Hz, 2H).

The following compounds in Table T-53 were synthesized using procedures similar to Compound 663 using the appropriate starting materials.

TABLE T-53

| | | | Exact | LCMS, |
|---|---|---|---|---|
| Compound | | | mass | found |
| No. | Structure | IUPAC name | (g/mol) | [M + H]⁺ |
| 664 | 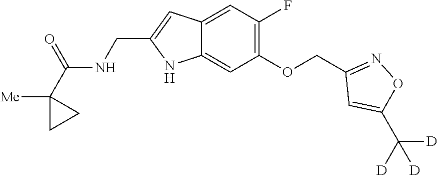 [SMILES: [2H]C([2H])([2H])c1cc(COc2cc3[nH]c(CNC(=O)C4(C)CC4)cc3cc2F)no1] | N-((5-fluoro-6-((5- (methyl-d3)isoxazol-3- yl)methoxy)-1H-indol- 2-yl)methyl)-1- methylcyclopropane-1- carboxamide | 360.17 | 361.2 |

### Example 92 (Compound 665)

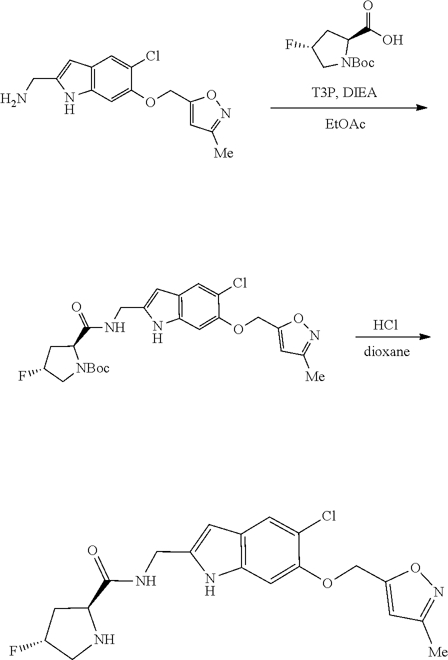 [SMILES: "EtOAc" ; Cc1cc(COc2cc3[nH]c(CN)cc3cc2Cl)on1 ; CC(C)(C)OC(=O)N1C[C@H](F)C[C@H]1C(=O)O ; Cc1cc(COc2cc3[nH]c(CNC(=O)[C@@H]4C[C@@H](F)CN4C(=O)OC(C)(C)C)cc3cc2Cl)on1 ; Cl ; Cc1cc(COc2cc3[nH]c(CNC(=O)[C@@H]4C[C@@H](F)CN4)cc3cc2Cl)on1]

### Step 1: tert-butyl (2S,4R)-2-(((5-chloro-6-((3-methylisoxazol-5-yl)methoxy)-1H-indol-2-yl)methyl)carbamoyl)-4-fluoropyrrolidine-1-carboxylate

A mixture of (5-chloro-6-((3-methylisoxazol-5-yl)methoxy)-1H-indol-2-yl)methanamine (300 mg, 1.028 mmol, 1 eq.), (2S,4R)-1-(tert-butoxycarbonyl)-4-fluoropyrrolidine-2-carboxylic acid (0.288 g, 1.234 mmol, 1.2 eq.), T3P (0.491 g, 0.982 mL, 50 w/v % in EtOAc, 1.542 mmol, 1.5 eq.) and DIEA (0.358 mL, 2.057 mmol, 2 eq.) in EtOAc (2.0 mL) were stirred at 20° C. After 4 hours, the mixture was diluted with H₂O (10 mL) and extracted with EtOAc (3×10 mL). The combined organic layers were dried over sodium sulfate and concentrated under reduced pressure. The resulting crude product was purified by column chromatography to give tert-butyl (2S,4R)-2-(((5-chloro-6-((3-methylisoxazol-5-yl)methoxy)-1H-indol-2-yl)methyl)carbamoyl)-4-fluoropyrrolidine-1-carboxylate. LC-MS (ESI) m/z calcd for C₂₄H₂₈ClFN₄O₅: 506.17; found 507.2 [M+H]⁺.

### Step 2: (2S,4R)—N-((5-chloro-6-((3-methylisoxazol-5-yl)methoxy)-1H-indol-2-yl)methyl)-4-fluoropyrrolidine-2-carboxamide

To a solution of tert-butyl (2S,4R)-2-(((5-chloro-6-((3-methylisoxazol-5-yl)methoxy)-1H-indol-2-yl)methyl)carbamoyl)-4-fluoropyrrolidine-1-carboxylate (380 mg, 0.75 mmol, 1 eq.) in 1,4-dioxane (1.5 mL) was added hydrochloric acid (0.937 mL, 4 M in dioxane, 3.748 mmol, 5 eq.). After 6 h, the reaction was diluted with EtOAc (50 mL), water (50 mL), and 1 M NaOH (5 mL). The mixture was extracted with EtOAc (2×50 mL), and the combined organic layers were dried over Na₂SO₄, filtered, and concentrated under reduced pressure. Purification by prep-HPLC gave (2S,4R)—N-((5-chloro-6-((3-methylisoxazol-5-yl)methoxy)-1H-indol-2-yl)methyl)-4-fluoropyrrolidine-2-carboxamide (Compound 665). LC-MS (ESI) m/z calcd for C₁₉H₂₀ClFN₄O₃: 406.12; found 407.2 [M+H]⁺. ¹H NMR (500 MHz, DMSO) δ 10.90 (d, J=2.1 Hz, 1H), 8.38 (t, J=6.0 Hz, 1H), 8.08 (s, 1H), 7.44 (s, 1H), 7.08 (s, 1H), 6.39 (s, 1H), 6.08 (d, J=2.0 Hz, 1H), 5.21 (s, 3H), 4.37-4.24 (m, 2H), 3.75 (t, J=8.2 Hz, 1H), 3.03 (ddd, J=23.0, 13.3, 2.2 Hz, 1H), 2.83 (ddd, J=38.8, 13.4, 3.2 Hz, 1H), 2.17 (s, 4H), 1.83 (dddd, J=39.8, 14.6, 8.4, 4.4 Hz, 1H).

The following compounds in Table T-54 were synthesized using procedures similar to Compound 665 using the appropriate starting materials.

TABLE T-54

| | | | Exact | LCMS, |
|---|---|---|---|---|
| Compound | | | mass | found |
| No. | Structure | IUPAC name | (g/mol) | [M + H]⁺ |
| 666 | 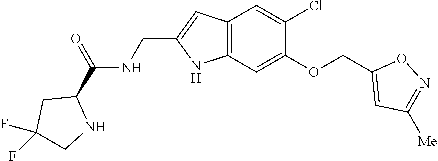 [SMILES: Cc1cc(COc2cc3[nH]c(CNC(=O)[C@@H]4CC(F)(F)CN4)cc3cc2Cl)on1] | (S)-N-((5-chloro-6-((3- methylisoxazol-5- yl)methoxy)-1H-indol- 2-yl)methyl)-4,4- difluoropyrrolidine-2- carboxamide | 424.11 | 425.2 |

### Example 93 (Compound 667)

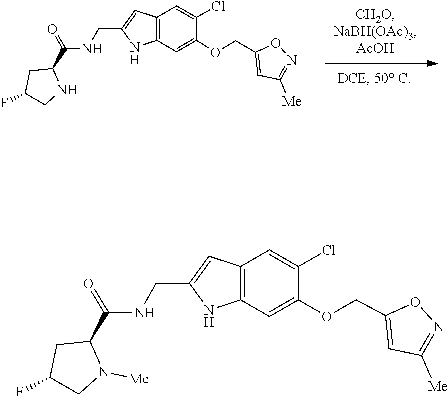 [SMILES: Cc1cc(COc2cc3[nH]c(CNC(=O)[C@@H]4C[C@@H](F)CN4)cc3cc2Cl)on1 ; Cc1cc(COc2cc3[nH]c(CNC(=O)[C@@H]4C[C@@H](F)CN4C)cc3cc2Cl)on1]

### Step 1: (2S,4R)—N-((5-chloro-6-((3-methylisoxazol-5-yl)methoxy)-1H-indol-2-yl)methyl)-4-fluoro-1-methylpyrrolidine-2-carboxamide

To a solution of (2S,4R)—N-((5-chloro-6-((3-methylisoxazol-5-yl)methoxy)-1H-indol-2-yl)methyl)-4-fluoropyrrolidine-2-carboxamide (70 mg, 0.172 mmol, 1 eq.), formaldehyde (0.026 g, 0.86 mmol, 5 eq.), and sodium triacetoxyborohydride (0.109 g, 0.516 mmol, 3 eq.) in DCE (0.86 mL) was added acetic acid (0.01 mL, 0.172 mmol, 1 eq.). The reaction was heated to 50° C. for 6 hours. The reaction was then cooled to room temperature and diluted with water (10 mL), and the mixture was extracted with EtOAc (3×10 mL). The combined organic layers were dried over sodium sulfate, filtered, and concentrated under reduced pressure. The resulting crude material was purified by prep-HPLC to give (2S,4R)—N-((5-chloro-6-((3-methylisoxazol-5-yl)methoxy)-1H-indol-2-yl)methyl)-4-fluoro-1-methylpyrrolidine-2-carboxamide (Compound 667). LC-MS (ESI) m/z calcd for C₂₀H₂₂ClFN₄O₃: 420.14; found 421.2 [M+H]⁺. ¹H NMR (500 MHz, DMSO) δ 10.90 (s, 1H), 8.26 (t, J=6.0 Hz, 1H), 7.45 (s, 1H), 7.09 (s, 1H), 6.40 (s, 1H), 6.09 (s, 1H), 5.22 (s, 2H), 4.31 (dq, J=15.4, 7.7 Hz, 2H), 3.37 (ddd, J=29.4, 11.9, 5.0 Hz, 2H), 3.11 (dd, J=9.2, 6.9 Hz, 1H), 2.58-2.48 (m, 1H), 2.26 (s, 3H), 2.18 (s, 3H), 2.15-2.09 (m, 1H), 1.95 (dddd, J=33.9, 14.6, 9.3, 5.7 Hz, 1H).

The following compounds in Table T-55 were synthesized using procedures similar to Compound 667 using the appropriate starting materials.

TABLE T-55

| | | | Exact | LCMS, |
|---|---|---|---|---|
| Compound | | | mass | found |
| No. | Structure | IUPAC name | (g/mol) | [M + H]⁺ |
| 668 | 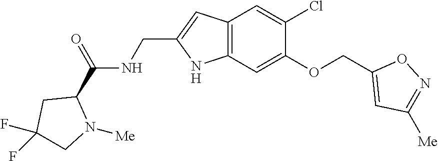 [SMILES: Cc1cc(COc2cc3[nH]c(CNC(=O)[C@@H]4CC(F)(F)CN4C)cc3cc2Cl)on1] | (S)-N-((5-chloro-6-((3- methylisoxazol-5- yl)methoxy)-1H-indol- 2-yl)methyl)-4,4- difluoro-1- methylpyrrolidine-2- carboxamide | 438.13 | 439.2 |

### Example 94 (Compound 669)

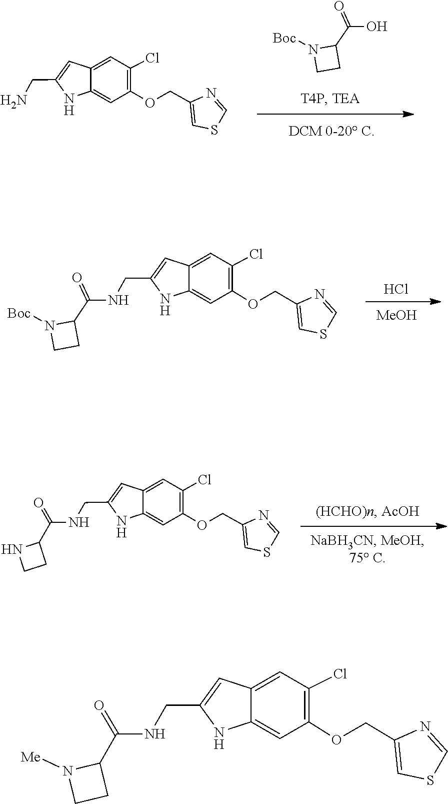

### Step 1: tert-butyl 2-(((5-chloro-6-(thiazol-4-ylmethoxy)-1H-indol-2-yl)methyl)carbamoyl)azetidine-1-carboxylate

To a mixture of (5-chloro-6-(thiazol-4-ylmethoxy)-1H-indol-2-yl)methanamine (400 mg, 1.36 mmol, 1.00 eq) and 1-(tert-butoxycarbonyl)azetidine-2-carboxylic acid (274 mg, 1.36 mmol, 1.00 eq) in DCM (10 mL) was added TEA (413 mg, 4.08 mmol, 3.00 eq). The reaction mixture as cooled to 0° C., and T4P (490 mg, 1.36 mmol, 1.00 equiv) was added dropwise. The reaction was then warmed to 25° C. for 2 h. The reaction mixture was quenched with aqueous sat. aq. NH₄Cl (60 mL). The resulting mixture was extracted with DCM (3×20 mL). The combined organic layers were washed with brine (20 mL), dried over anhydrous Na₂SO₄, filtered, and concentrated under reduced pressure. Purification by column chromatography to give tert-butyl 2-(((5-chloro-6-(thiazol-4-ylmethoxy)-1H-indol-2-yl)methyl)carbamoyl)azetidine-1-carboxylate.

### Step 2: N-((5-chloro-6-(thiazol-4-ylmethoxy)-1H-indol-2-yl)methyl)azetidine-2-carboxamide

To a solution of tert-butyl 2-(((5-chloro-6-(thiazol-4-ylmethoxy)-1H-indol-2-yl)methyl)carbamoyl)azetidine-1-carboxylate (400 mg, 839 μmol, 1.00 eq) in MeOH (5 mL) was added HCl (4 M in MeOH, 4 mL, 19.1 eq), and the reaction was stirred at 25° C. for 1 h. The reaction mixture was concentrated in vacuo to give N-((5-chloro-6-(thiazol-4-ylmethoxy)-1H-indol-2-yl)methyl)azetidine-2-carboxamide. LC-MS (ESI) m/z calcd for C₁₇H₁₇ClN₄O₂S: 376.08; found: 377.1 [M+H]⁺.

### Step 3: N-((5-chloro-6-(thiazol-4-ylmethoxy)-1H-indol-2-yl)methyl)-1-methylazetidine-2-carboxamide

To a solution of N-((5-chloro-6-(thiazol-4-ylmethoxy)-1H-indol-2-yl)methyl)azetidine-2-carboxamide (140 mg, 339 μmol, 1.00 eq) in MeOH (4 mL) were added TEA (68.6 mg, 677 μmol, 2.00 eq), AcOH (0.2 mL), formaldehyde (51.0 mg, 1.69 mmol, 5.00 eq) and NaBH₃CN (32.0 mg, 508 μmol, 1.50 eq) at 25° C. The reaction mixture was then heated to 75° C. and stirred for 16 h. The reaction mixture was then cooled to 25° C. and quenched with aqueous sat. aq. NaHCO₃ (30 mL). The resulting mixture was extracted with DCM:i-PrOH (3:1, 5×10 mL). The combined organic layers were washed with brine (15 mL), dried over anhydrous Na₂SO₄, filtered, and concentrated under reduced pressure. Purification by prep-HPLC gave N-((5-chloro-6-(thiazol-4-ylmethoxy)-1H-indol-2-yl)methyl)-1-methylazetidine-2-carboxamide (Compound 669). LC-MS (ESI) m/z calcd for C₁₈H₁₉ClN₄O₂S: 390.09; found: 391.1 [M+H]⁺. ¹H NMR (400 MHz, DMSO-d₆) δ 10.92 (s, 1H), 9.15 (d, J=2.0 Hz, 1H), 8.20 (t, J=6.0 Hz, 1H), 7.77 (s, 1H), 7.51 (s, 1H), 7.20 (s, 1H), 6.15 (s, 1H), 5.28 (s, 2H), 4.39 (d, J=6.0 Hz, 2H), 3.39 (t, J=8.4 Hz, 1H), 3.30-3.27 (m, 1H), 2.86-2.84 (m, 1H), 2.26 (s, 3H), 2.26-2.18 (m, 1H), 2.00-1.95 (m, 1H).

### Biological Examples

### Example B-1

CHO cells with doxycycline-inducible expression of human SLC6A19 and TMEM27 were maintained in T150 cell culture treated flasks using cell culture media, which consisted of 1× DMEM/F12 (Gibco, 11330057), 10% Tet System-Approved FBS (Gibco, A4736401), 100 U/mL Penicillin-Streptomycin (Gibco, 15140122), 1× GlutaMAX™ Supplement (Gibco, 35050061), 1 mM Sodium Pyruvate (Gibco, 11360070), 1.1 mg/mL Sodium Bicarbonate (Gibco, 25080094), 5 μg/mL Blasticidin (InvivoGen, ant-bl-1), 150 μg/mL Hygromycin B Gold (InvivoGen, ant-hg-5), and 500 μg/mL G418 (Sigma-Aldrich, G8168-100 mL). In preparation for the assay, this media was aspirated and 5 mL of TrypLE™ Express Enzyme (Gibco, 12605010) was added to each flask to initiate cell dissociation. The flasks were then incubated (humidified, 37° C. with 5% CO₂) for 2 minutes. After incubation, 10 mL of pre-warmed cell culture media was added to each flask. The cell suspension was collected and centrifuged for 5 minutes at 200×g. The supernatant was aspirated and the pellet was resuspended in 5 mL pre-warmed cell culture media. The resuspended cells were counted using the Countess Cell Counting Chamber (Invitrogen) and the cell concentration of the suspension was readjusted to 1 million live cells/mL using pre-warmed cell culture media. 25 uL/well (25,000 cells/well) of readjusted cell suspension was dispensed into Poly-D-Lysine 384-well white/clear plates (Corning, 354660) using a MultiDrop Combi (Thermo Electron Corp). To induce expression of Human SLC6A19 and TMEM27, 25 uL/well of cell culture media containing 2 μg/mL doxycycline was added to the first 23 columns of each plate resulting in a final concentration of 1 μg/mL doxycycline. 25 μL/well of cell culture media was added to the last column of each plate, to act as negative control wells for Human SLC6A19 and TMEM27 expression. Assay plates were then incubated (humidified, 37° C. with 5% CO₂) for 18 hours. After incubation, the media was aspirated and the wells were washed 4 times with pre-warmed HBSS (Sigma-Aldrich, H8264-1L) using an Apricot S-Pipette S2 (Apricot Designs), such that 5 uL/well HBSS was remaining at the end of each wash. 10 μL/well of test compound, a positive control compound to define 0% activity (i.e., 100% inhibition), or DMSO diluted in HBSS (Sigma-Aldrich, H8264-1L) was then added into each well using the Apricot S-Pipette S2 (Apricot Designs). At this step, the final in-well DMSO concentration in the assay plate is 0.5%. All compounds were plated in a duplicate 8-point dilution series that consisted of 3-fold dilutions. The plates were incubated with compound (humidified, 37° C. with 5% CO₂) for 1 hour. Afterwards, 15 uL/well of 3.2 mM hot:cold leucine solution (1:15000 ratio of L-[3,4,5-3H(N)] Leucine (Perkin Elmer, NET460001MC) to L-Leucine (Sigma-Aldrich, L8912-25G) diluted with HBSS (Sigma-Aldrich, H8264-1L)) was added using the Viaflo 384 (Integra), for a final concentration of 1.6 mM hot:cold leucine solution. The plates were then incubated (humidified, 37° C. with 5% CO₂) for 20 minutes to conduct the uptake reaction. Immediately after the incubation, the plates were washed 4 times with HBSS (Sigma-Aldrich, H8264-1L) using the ELx405 Microplate Washer (Biotek) to terminate the uptake reaction. After the washes, 50 uL/well Ultima Gold Scintillation Cocktail (Perkin Elmer, 6013326) was added to each well using the Viaflo 384 (Integra). The plates were then incubated (humidified, 37° C. with 5% CO₂) on a plate shaker at 200 rpm for 2 hours. After incubation, the plates were removed from the shaker and kept at room temperature for 15 minutes.

Liquid scintillation analysis was performed using the MicroBeta2 Microplate Counter (Perkin Elmer). Activity (%) was derived by normalizing the average of positive control compound treated wells to 0% and the average of DMSO (negative control) wells to 100%. Collaborative Drug Discovery software was used for graphing data.

TABLE B1

| Cmpd | SLC6A19_Radioligand |
|---|---|
| # | Uptake Assay IC50 (μM) |
| 6 | 0.175 |
| 8 | 0.119 |
| 11 | 0.118 |
| 12 | 0.079 |
| 13 | 0.637 |
| 14 | 0.111 |
| 15 | 0.363 |
| 16 | 0.172 |
| 17 | 0.362 |
| 18 | 0.078 |
| 19 | 0.341 |
| 21 | 0.532 |
| 22 | 2.24 |
| 23 | 1.43 |
| 24 | 0.412 |
| 25 | 0.124 |
| 26 | 0.149 |
| 27 | 0.084 |
| 28 | 0.17 |
| 29 | 0.060 |
| 30 | 0.263 |
| 31 | 0.419 |
| 32 | 0.133 |
| 33 | 0.172 |
| 34 | 0.602 |
| 35 | 0.902 |
| 36 | 0.716 |
| 41 | 0.261 |
| 45 | 0.180 |
| 46 | 0.070 |
| 58 | 0.162 |
| 59 | 0.214 |
| 60 | 0.254 |
| 61 | 0.297 |
| 63 | 0.299 |
| 64 | 0.078 |
| 65 | 0.152 |
| 66 | 0.070 |
| 67 | 0.169 |
| 68 | 0.119 |
| 69 | 0.148 |
| 70 | 0.117 |
| 71 | 0.111 |
| 72 | 1.24 |
| 73 | 1.47 |
| 74 | 0.202 |
| 76 | 0.100 |
| 79 | 0.272 |
| 80 | 0.350 |
| 81 | 0.150 |
| 82 | 0.149 |
| 83 | 0.100 |
| 84 | 0.690 |
| 85 | 0.402 |
| 86 | 0.490 |
| 87 | 0.365 |
| 88 | 0.477 |
| 89 | 0.567 |
| 90 | 0.870 |
| 91 | 0.524 |
| 92 | 0.214 |
| 93 | 0.177 |
| 96 | 0.261 |
| 97 | 0.421 |
| 98 | 0.210 |
| 99 | 0.158 |
| 100 | 0.201 |
| 101 | 4.36 |
| 102 | 17.6 |
| 103 | 0.114 |
| 113 | 0.284 |
| 114 | 0.184 |
| 115 | 0.230 |
| 116 | 0.157 |
| 117 | 0.267 |
| 118 | 1.11 |
| 120 | 0.375 |
| 121 | 0.282 |
| 122 | 0.360 |
| 123 | 0.340 |
| 124 | 0.450 |
| 125 | 0.198 |
| 126 | 0.097 |
| 127 | 0.098 |
| 128 | 0.070 |
| 129 | 0.080 |
| 130 | 0.085 |
| 131 | 0.074 |
| 132 | 0.136 |
| 133 | 0.082 |
| 134 | 0.187 |
| 135 | 0.082 |
| 136 | 0.083 |
| 137 | 0.183 |
| 138 | 0.353 |
| 139 | 0.310 |
| 140 | 0.148 |
| 141 | 0.087 |
| 142 | 0.069 |
| 143 | 0.182 |
| 144 | 0.119 |
| 145 | 0.073 |
| 146 | 0.060 |
| 147 | 0.067 |
| 148 | 0.154 |
| 149 | 0.085 |
| 150 | 0.120 |
| 151 | 0.182 |
| 152 | 0.531 |
| 153 | 1.96 |
| 172 | 0.157 |
| 177 | 0.307 |
| 184 | 0.144 |
| 185 | 1.05 |
| 186 | 0.968 |
| 189 | 9.83 |
| 190 | 0.185 |
| 191 | 1.68 |
| 192 | 2.51 |
| 193 | 9.88 |
| 194 | 6.78 |
| 195 | 14.8 |
| 196 | 0.207 |
| 197 | 0.154 |
| 198 | 2.05 |
| 199 | 1.37 |
| 200 | 0.752 |
| 201 | 5.07 |
| 202 | 1.56 |
| 203 | 1.09 |
| 204 | 0.536 |
| 205 | 0.787 |
| 206 | 0.217 |
| 207 | 0.926 |
| 208 | 0.133 |
| 209 | 0.13 |
| 210 | 0.293 |
| 211 | 0.0877 |
| 212 | 0.429 |
| 213 | 0.192 |
| 214 | 0.152 |
| 215 | 0.977 |
| 216 | 1.41 |
| 218 | 0.0694 |
| 219 | 0.134 |
| 220 | 1.55 |
| 221 | 0.288 |
| 222 | 3.17 |
| 223 | 0.203 |
| 224 | 0.545 |
| 225 | 0.156 |
| 226 | 1.12 |
| 227 | 0.317 |
| 228 | 3.17 |
| 229 | 0.157 |
| 230 | 0.0696 |
| 231 | 0.139 |
| 232 | 0.101 |
| 233 | 0.112 |
| 234 | 0.36 |
| 235 | 0.283 |
| 236 | 2.99 |
| 237 | 1.41 |
| 238 | 1.45 |
| 239 | 5.88 |
| 240 | 0.49 |
| 241 | 1.02 |
| 242 | 0.674 |
| 243 | 0.336 |
| 244 | 0.391 |
| 245 | 1.19 |
| 246 | 0.411 |
| 247 | 0.77 |
| 248 | 0.627 |
| 249 | 0.545 |
| 250 | 0.383 |
| 251 | 0.194 |
| 252 | 0.628 |
| 253 | 0.606 |
| 254 | 0.294 |
| 255 | 1.36 |
| 256 | 0.14 |
| 257 | 0.756 |
| 258 | 2.67 |
| 259 | 0.121 |
| 260 | 2.35 |
| 261 | 0.105 |
| 262 | 0.472 |
| 263 | 0.174 |
| 264 | 0.243 |
| 265 | 0.12 |
| 266 | 0.142 |
| 267 | 0.72 |
| 268 | 0.421 |
| 269 | 0.0666 |
| 270 | 0.11 |
| 271 | 0.515 |
| 272 | 0.0789 |
| 273 | 0.567 |
| 274 | 0.175 |
| 275 | 0.35 |
| 276 | 0.094 |
| 277 | 0.279 |
| 278 | 0.108 |
| 279 | 0.383 |
| 280 | 5.48 |
| 281 | 2.28 |
| 282 | 2.67 |
| 283 | >8.95 |
| 284 | 0.172 |
| 285 | 0.322 |
| 286 | 0.91 |
| 287 | 2.4 |
| 288 | 3.26 |
| 289 | 2.02 |
| 290 | 5.29 |
| 291 | 0.197 |
| 292 | 0.666 |
| 293 | 0.5 |
| 294 | 0.135 |
| 295 | 1.61 |
| 296 | 0.976 |
| 297 | 1.02 |
| 298 | 0.921 |
| 299 | >9.06 |
| 300 | 0.321 |
| 301 | 0.327 |
| 302 | 7.11 |
| 303 | 0.814 |
| 304 | 0.718 |
| 305 | 0.209 |
| 306 | 0.633 |
| 307 | 0.579 |
| 308 | 0.168 |
| 309 | 1.75 |
| 310 | 2.52 |
| 311 | 0.129 |
| 312 | 0.317 |
| 313 | 0.125 |
| 314 | 0.375 |
| 315 | 0.148 |
| 316 | 0.284 |
| 317 | 0.119 |
| 318 | 0.226 |
| 319 | 0.273 |
| 320 | 1.08 |
| 321 | 0.891 |
| 322 | 0.275 |
| 323 | 1.81 |
| 324 | 1.12 |
| 325 | 3.73 |
| 326 | 0.196 |
| 327 | 0.223 |
| 328 | 0.147 |
| 329 | 0.167 |
| 330 | 0.167 |
| 331 | 0.452 |
| 332 | 0.269 |
| 333 | 0.206 |
| 334 | 0.519 |
| 335 | 0.126 |
| 336 | 1.19 |
| 337 | 1.19 |
| 338 | 0.159 |
| 339 | 0.328 |
| 340 | 0.277 |
| 341 | 0.273 |
| 342 | 0.258 |
| 343 | 3.16 |
| 344 | 3.91 |
| 345 | 0.0627 |
| 346 | >9.54 |
| 347 | 1.05 |
| 348 | 0.516 |
| 349 | 2.93 |
| 350 | >8.38 |
| 351 | 2.47 |
| 352 | 0.0675 |
| 353 | 1.93 |
| 354 | >5.28 |
| 355 | 6.6 |
| 356 | 0.389 |
| 357 | 0.413 |
| 358 | 0.319 |
| 359 | 0.231 |
| 360 | 0.432 |
| 361 | 0.286 |
| 362 | 0.983 |
| 363 | 0.332 |
| 364 | 0.69 |
| 365 | 0.991 |
| 366 | 1.53 |
| 367 | 1.24 |
| 368 | >5.48 |
| 369 | 0.125 |
| 370 | 0.486 |
| 371 | 0.141 |
| 372 | 0.253 |
| 373 | 0.472 |
| 374 | 0.412 |
| 375 | 0.948 |
| 376 | 2.12 |
| 377 | 0.847 |
| 378 | 5.51 |
| 379 | 0.382 |
| 380 | 1.13 |
| 381 | 0.339 |
| 382 | 0.42 |
| 383 | 0.178 |
| 384 | 1.57 |
| 385 | 0.549 |
| 386 | 0.174 |
| 387 | 0.209 |
| 388 | 0.336 |
| 389 | 0.353 |
| 390 | 0.34 |
| 391 | 0.225 |
| 392 | 2.83 |
| 393 | 1.46 |
| 394 | 0.676 |
| 395 | 2.14 |
| 396 | 0.644 |
| 397 | 0.492 |
| 398 | 0.599 |
| 399 | 0.429 |
| 400 | 0.556 |
| 401 | 0.342 |
| 402 | 0.29 |
| 403 | 0.407 |
| 404 | 0.268 |
| 405 | 0.305 |
| 406 | 0.347 |
| 407 | 0.619 |
| 408 | 0.478 |
| 409 | 1.18 |
| 410 | 0.386 |
| 411 | 0.313 |
| 412 | 0.289 |
| 413 | 0.276 |
| 414 | 0.339 |
| 415 | 0.384 |
| 416 | 0.379 |
| 417 | 0.394 |
| 418 | 0.163 |
| 419 | 0.435 |
| 420 | 0.355 |
| 421 | 0.359 |
| 422 | 0.337 |
| 423 | 0.198 |
| 424 | 1.26 |
| 425 | 0.315 |
| 426 | 0.351 |
| 427 | 0.634 |
| 428 | 0.161 |
| 429 | 0.215 |
| 430 | 0.137 |
| 431 | 0.244 |
| 432 | 0.384 |
| 433 | 0.566 |
| 434 | 0.263 |
| 435 | 0.133 |
| 436 | 0.097 |
| 437 | 0.382 |
| 438 | 0.0709 |
| 439 | 0.327 |
| 440 | 0.111 |
| 441 | 0.0956 |
| 442 | 0.0752 |
| 443 | 0.0642 |
| 444 | 0.0774 |
| 445 | 0.444 |
| 446 | 0.221 |
| 447 | 0.208 |
| 448 | 2.75 |
| 449 | 0.187 |
| 450 | 0.0951 |
| 451 | 0.237 |
| 452 | 0.145 |
| 453 | 0.859 |
| 454 | 0.787 |
| 455 | 3.53 |
| 456 | 4.26 |
| 457 | 3.64 |
| 458 | 7.67 |
| 459 | 0.505 |
| 460 | 1.36 |
| 461 | 2.7 |
| 462 | 2 |
| 463 | >8.73 |
| 464 | 1.35 |
| 465 | 6.21 |
| 466 | 2.96 |
| 467 | 0.145 |
| 468 | 0.422 |
| 469 | 0.392 |
| 470 | 1.31 |
| 471 | 0.599 |
| 472 | 0.304 |
| 473 | 0.293 |
| 474 | 0.553 |
| 475 | 0.432 |
| 476 | 0.408 |
| 477 | 0.383 |
| 478 | 0.416 |
| 479 | 1.67 |
| 480 | 0.372 |
| 481 | 0.318 |
| 482 | 0.149 |
| 483 | 0.17 |
| 484 | 0.199 |
| 485 | 0.578 |
| 486 | 0.387 |
| 487 | 0.367 |
| 488 | 0.161 |
| 489 | 0.325 |
| 490 | 0.178 |
| 491 | 0.814 |
| 492 | 0.359 |
| 493 | 1.44 |
| 494 | 0.148 |
| 495 | 0.397 |
| 496 | 0.43 |
| 497 | 0.197 |
| 498 | 0.159 |
| 499 | 0.213 |
| 500 | 0.195 |
| 501 | 0.312 |
| 502 | 2.13 |
| 503 | 0.317 |
| 504 | 0.391 |
| 505 | 0.203 |
| 506 | 0.331 |
| 507 | 0.327 |
| 508 | 0.293 |
| 509 | 1.52 |
| 510 | 0.385 |
| 511 | 0.0861 |
| 512 | 6.78 |
| 513 | 0.0862 |
| 514 | 0.408 |
| 515 | 0.116 |
| 516 | 0.0889 |
| 517 | 0.243 |
| 518 | 0.179 |
| 519 | 0.118 |
| 520 | 6.73 |
| 521 | >7.97 |
| 522 | 0.199 |
| 523 | 0.208 |
| 524 | 0.4 |
| 525 | 0.157 |
| 526 | 0.156 |
| 527 | 4.61 |
| 528 | 0.278 |
| 529 | 0.192 |
| 530 | 0.171 |
| 531 | 0.295 |
| 532 | 0.509 |
| 533 | 0.332 |
| 534 | 3.04 |
| 535 | 1.65 |
| 536 | 1.36 |
| 537 | 0.108 |
| 538 | 0.0594 |
| 539 | 0.123 |
| 540 | 0.131 |
| 541 | 0.141 |
| 542 | 0.123 |
| 543 | 0.145 |
| 544 | 0.119 |
| 545 | 3.54 |
| 546 | 4.48 |
| 547 | 0.363 |
| 548 | 0.1 |
| 549 | 0.426 |
| 550 | 0.552 |
| 551 | 0.954 |
| 552 | 0.34 |
| 553 | 0.342 |
| 554 | 0.214 |
| 555 | 3.04 |
| 556 | 1.2 |
| 557 | >8.24 |
| 558 | 1.03 |
| 559 | 0.196 |
| 560 | 0.0669 |
| 561 | 0.57 |
| 562 | 0.0833 |
| 563 | 0.0822 |
| 564 | 0.174 |
| 565 | 0.286 |
| 566 | 0.146 |
| 567 | 0.182 |
| 568 | 0.337 |
| 569 | 0.121 |
| 570 | 0.0804 |
| 571 | 0.161 |
| 572 | 0.113 |
| 573 | 1.39 |
| 574 | 3.6 |
| 575 | 3.05 |
| 576 | 0.824 |
| 577 | 1.44 |
| 578 | >5.55 |
| 579 | 0.587 |
| 580 | 0.62 |
| 581 | 0.657 |
| 582 | 0.455 |
| 583 | 1.52 |
| 584 | 0.889 |
| 585 | >9.23 |
| 586 | 0.176 |
| 587 | 0.26 |
| 588 | 3.67 |
| 589 | 4.76 |
| 590 | 0.145 |
| 591 | 0.184 |
| 592 | 0.163 |
| 593 | 0.22 |
| 594 | 0.12 |
| 595 | 0.24 |
| 596 | 0.336 |
| 597 | 0.307 |
| 598 | 0.358 |
| 599 | 0.163 |
| 600 | 0.145 |
| 601 | 0.174 |
| 602 | 0.82 |
| 603 | 0.345 |
| 604 | 0.391 |
| 605 | 0.143 |
| 606 | 0.32 |
| 607 | >6.44 |
| 608 | 0.304 |
| 609 | 0.339 |
| 610 | 0.991 |
| 611 | 0.842 |
| 612 | 0.152 |
| 613 | 0.0824 |
| 614 | 3.26 |
| 615 | 2.07 |
| 616 | 2.73 |
| 617 | 3.52 |
| 618 | 7.51 |
| 619 | >8.44 |
| 620 | 0.182 |
| 621 | 1.77 |
| 622 | 0.0848 |
| 623 | 0.532 |
| 624 | 0.104 |
| 625 | 0.0902 |
| 626 | 0.765 |
| 627 | >8.73 |
| 628 | 2.67 |
| 629 | 1.07 |
| 630 | 0.156 |
| 631 | 0.21 |
| 632 | 1.47 |
| 633 | 0.181 |
| 634 | 0.404 |
| 635 | 0.193 |
| 636 | 0.164 |
| 637 | 0.376 |
| 638 | 0.264 |
| 639 | 0.112 |
| 640 | 0.666 |
| 641 | 2.1 |
| 642 | 2.74 |
| 643 | 0.631 |
| 644 | 0.583 |
| 645 | 0.955 |
| 646 | 0.539 |
| 647 | 0.68 |
| 648 | 0.785 |
| 649 | 0.326 |
| 650 | 1.01 |
| 651 | 0.34 |
| 652 | 0.365 |
| 653 | 0.387 |
| 654 | 0.537 |
| 655 | >7.30 |
| 656 | 8.47 |
| 657 | 0.654 |
| 658 | 0.59 |
| 659 | 1.97 |
| 660 | 1.58 |
| 661 | 1 |
| 662 | 0.123 |
| 663 | 0.134 |
| 664 | 0.187 |
| 665 | 1.56 |
| 666 | 2.99 |
| 667 | 0.524 |
| 668 | 1.15 |
| 669 | 3.88 |

### Example B-2

Assay ready vials of CHO cells with doxycycline inducible expression of human SLC6A19 and TMEM27 were thawed, plated in T175 flasks containing DMEM/F12 media, 10% FBS, 1% Penicillin-Streptomycin, 1× UltraGlutamate, 1 mM Sodium Pyruvate, Sodium Bicarbonate (7.5 mL of 7.5% solution in 500 mL media final), 5 μg/mL Blasticidin, 150 μg/mL Hygromycin B Gold, and 500 μg/mL G418, and incubated (humidified, 37° C. with 5% CO2) for 48 hours. Media was then removed, flasks were washed with 20 mL of DPBS, and 5 mL of TrypLE™ Express was added to each flask to initiate cell dissociation. After 5 minutes, 10 mL of cell culture media was added to each flask and mixed. The cell suspension was collected and centrifuged for 5 minutes at 200×g. The supernatant was removed, and the pellet was resuspended in 10 mL of cell culture media. Resuspended cells were counted using a Vi-Cell. 30 μL/well (20,000 cells/well) of cell suspension was added to Poly-D-Lysine 384-well black/clear plates. Plates were incubated (humidified, 37° C. with 5% CO2) for 24 hours. To induce expression of human SLC6A19 and TMEM27, 10 μL of assay culture media containing 4 μg/mL doxycycline was added to each well. Assay plates were then incubated (humidified, 37° C. with 5% CO2) overnight. After incubation, the media was removed, and wells were washed 3 times with 40 μL of warmed DPBS. Next, 10 μL of warmed assay buffer (137 mM NaCl, 5 mM KCl, 1 mM CaCl2), 1 mM MgCl2, 10 mM HEPES, 10 mM glucose, pH 7.2) was added to each well. 10 μL/well of test compound or positive control compound to define 0% activity (i.e., 100% inhibition), was then added into each well. All compounds were plated in a duplicate 10-point dilution series that consisted of 2-fold dilutions. The plates were incubated (humidified, 37° C. with 5% C2) with compound for 20 minutes. Afterwards, 20 μL/well of 2 mM 13C6,15N-1-isoleucine was added to each well and plates were incubated again (humidified, 37° C. with 5% C02) for another 20 minutes. Assay buffer was then removed, and plates were washed. 70 μL/well of stop solution (75% acetonitrile, 25% HPLC grade water, 250 nM 1-leucine-1-13C (RapidFire standard)) was added to each well. The plates were sealed and then placed in a −20° C. freezer for 20 minutes. The plates were thawed at room temperature for 20 minutes and then centrifuge for 20 minutes at 3000 rpm. Using an ABSciex API4000 mass spectrometer, processed samples were loaded onto the graphitic carbon SPE-column. The time for aspiration, load/wash, elution and re-equilibration time was 600, 1500, 3000 and 500 milliseconds, respectively. Peak areas were calculated using RapidFire Integrator software.

TABLE B2

| Cmpd | SLC6A19_RapidFire |
|---|---|
| # | Uptake Assay IC50 (μM) |
| 1 | 5.54 |
| 2 | 0.992 |
| 3 | 0.504 |
| 4 | 1.83 |
| 5 | 0.053 |
| 6 | 0.148 |
| 7 | 0.225 |
| 8 | 0.051 |
| 9 | 0.271 |
| 10 | 0.130 |
| 11 | 0.076 |
| 12 | 0.144 |
| 13 | 0.764 |
| 14 | 0.120 |
| 18 | 0.091 |
| 20 | 0.168 |
| 23 | 0.502 |
| 24 | 0.222 |
| 25 | 0.033 |
| 26 | 0.166 |
| 27 | 0.092 |
| 28 | 0.079 |
| 29 | 0.045 |
| 30 | 0.155 |
| 31 | 0.504 |
| 32 | 0.058 |
| 33 | 0.221 |
| 34 | 0.615 |
| 36 | 0.316 |
| 37 | 0.625 |
| 38 | 0.044 |
| 39 | 0.370 |
| 40 | 0.064 |
| 41 | 0.071 |
| 42 | 1.62 |
| 43 | 0.192 |
| 44 | 0.838 |
| 45 | 0.075 |
| 46 | 0.061 |
| 47 | 0.698 |
| 48 | 0.493 |
| 49 | 0.412 |
| 50 | 0.193 |
| 51 | 4.39 |
| 52 | 2.11 |
| 53 | 4.82 |
| 54 | 0.155 |
| 55 | 0.427 |
| 56 | 1.95 |
| 57 | 0.577 |
| 58 | 0.028 |
| 59 | 0.153 |
| 60 | 0.071 |
| 62 | 0.493 |
| 63 | 0.056 |
| 64 | 0.024 |
| 65 | 0.046 |
| 66 | 0.027 |
| 67 | 0.117 |
| 68 | 0.117 |
| 69 | 0.078 |
| 70 | 0.062 |
| 74 | 0.157 |
| 75 | 0.464 |
| 76 | 0.034 |
| 77 | 0.355 |
| 78 | 5.18 |
| 81 | 0.166 |
| 82 | 0.045 |
| 83 | 0.050 |
| 90 | 1.20 |
| 94 | 0.0434 |
| 95 | 0.267 |
| 96 | 0.354 |
| 97 | 0.909 |
| 98 | 0.274 |
| 99 | 0.081 |
| 100 | 0.397 |
| 104 | 0.452 |
| 105 | 0.644 |
| 106 | 0.613 |
| 107 | 5.89 |
| 108 | 5.57 |
| 109 | 0.515 |
| 110 | 0.464 |
| 111 | 0.586 |
| 112 | 0.193 |
| 113 | 0.174 |
| 114 | 0.158 |
| 119 | 0.485 |
| 121 | 0.590 |
| 122 | 0.783 |
| 123 | 0.576 |
| 124 | 1.32 |
| 125 | 0.145 |
| 126 | 0.171 |
| 128 | 0.032 |
| 129 | 0.044 |
| 130 | 0.065 |
| 131 | 0.050 |
| 132 | 0.103 |
| 133 | 0.045 |
| 134 | 0.084 |
| 135 | 0.049 |
| 136 | 0.038 |
| 145 | 0.043 |
| 146 | 0.036 |
| 147 | 0.033 |
| 148 | 0.031 |
| 149 | 0.034 |
| 150 | 0.036 |
| 151 | 0.173 |
| 152 | 2.06 |
| 154 | 1.36 |
| 155 | 0.311 |
| 156 | 0.198 |
| 157 | 0.043 |
| 158 | 0.188 |
| 159 | 0.493 |
| 160 | >50.0 |
| 161 | 0.068 |
| 162 | 16.3 |
| 163 | 0.920 |
| 164 | 0.098 |
| 165 | 2.18 |
| 166 | 0.048 |
| 167 | 7.50 |
| 168 | 7.44 |
| 169 | 0.083 |
| 170 | 0.187 |
| 171 | 1.73 |
| 172 | 0.041 |
| 173 | 0.926 |
| 174 | 2.08 |
| 175 | 0.038 |
| 176 | 0.528 |
| 177 | 0.275 |
| 178 | 0.511 |
| 179 | 1.42 |
| 180 | 0.563 |
| 181 | 0.183 |
| 182 | 0.091 |
| 183 | 0.039 |
| 184 | 0.059 |
| 187 | 0.178 |
| 188 | 4.45 |
| 190 | 0.294 |
| 191 | 1.22 |
| 194 | 4.94 |
| 196 | 0.460 |
| 205 | 0.714 |
| 209 | 0.117 |
| 211 | 0.0381 |
| 214 | 0.193 |
| 215 | 3.89 |
| 216 | 3.74 |
| 217 | 3.42 |
| 218 | 0.0275 |
| 219 | 0.103 |
| 220 | 3.48 |
| 221 | 0.468 |
| 222 | 6.61 |
| 223 | 0.796 |
| 225 | 0.0808 |
| 226 | 0.96 |
| 227 | 0.486 |
| 228 | 11.2 |
| 229 | 0.204 |
| 230 | 0.0229 |
| 231 | 0.0493 |
| 232 | 0.0412 |
| 233 | 0.057 |
| 234 | 0.455 |
| 235 | 0.521 |
| 236 | >19.3 |
| 320 | 0.48 |
| 321 | 0.379 |
| 322 | 0.138 |
| 323 | 0.531 |
| 324 | 0.476 |
| 326 | 0.125 |
| 327 | 0.0891 |
| 330 | 0.123 |
| 332 | 0.187 |
| 341 | 0.194 |
| 342 | 0.215 |
| 343 | 2.48 |
| 344 | >50.0 |
| 346 | >50.0 |
| 351 | 1.54 |
| 352 | 0.469 |
| 357 | 0.434 |
| 379 | 0.24 |
| 389 | 0.485 |
| 390 | 0.402 |
| 392 | 1.62 |
| 393 | 0.781 |
| 401 | 0.534 |
| 402 | 0.175 |
| 403 | 0.519 |
| 404 | 0.147 |
| 405 | 0.367 |
| 406 | 0.172 |
| 407 | 0.593 |
| 408 | 0.498 |
| 409 | 2.29 |
| 410 | 0.578 |
| 411 | 0.217 |
| 412 | 0.154 |
| 413 | 0.239 |
| 414 | 0.281 |
| 415 | 0.0934 |
| 419 | 0.159 |
| 421 | 0.131 |
| 424 | 0.491 |
| 425 | 0.115 |
| 426 | 0.217 |
| 430 | 0.0686 |
| 431 | 0.343 |
| 432 | 0.253 |
| 433 | 1.5 |
| 438 | 0.0268 |
| 452 | 0.0636 |
| 460 | 0.732 |
| 461 | 0.989 |
| 470 | 1.82 |
| 471 | 1.09 |
| 472 | 0.397 |
| 475 | 0.138 |
| 478 | 0.172 |
| 479 | 0.545 |
| 481 | 0.297 |
| 493 | 1.14 |
| 494 | 0.0357 |
| 495 | 0.24 |
| 496 | 0.424 |
| 497 | 0.184 |
| 498 | 0.0902 |
| 499 | 0.0554 |
| 500 | 0.257 |
| 501 | 1.3 |
| 502 | 1.78 |
| 503 | 0.0667 |
| 504 | 0.406 |
| 523 | 0.239 |
| 524 | 0.57 |
| 528 | 0.292 |
| 529 | 0.154 |
| 530 | 0.208 |
| 531 | 0.164 |
| 532 | 0.632 |
| 533 | 0.33 |
| 547 | 0.314 |
| 548 | 0.0545 |
| 549 | 0.833 |
| 550 | 1.04 |
| 551 | 1.76 |
| 590 | 0.0518 |
| 592 | 0.0931 |
| 602 | 0.609 |
| 603 | 0.263 |
| 608 | 0.457 |
| 624 | 0.0309 |
| 627 | 12.1 |
| 628 | 3.02 |
| 630 | 0.113 |
| 631 | 0.149 |
| 632 | >2.05 |
| 634 | 0.168 |
| 645 | 0.524 |
| 648 | 0.344 |
| 649 | 0.217 |

### Example B-3

B6.Pah^{enu2} mice can be used for measurement of plasma phenylalanine reduction and urine amino acid excretion and C57B16 mice can be used for measurement of urine amino acid excretion by compound. This example shows that the compounds of the present disclosure can be used to show reduction of plasma phenylalanine and increase in urinary excretion of amino acids.

B6.Pah^{enu2} mice, a model of phenylketonuria, is obtained from JAX (strain #029218). These mice have a mutation in the phenylalanine hydroxylase (Pah) gene, resulting in hyperphenylalanemia. Male and female B6.Pah^{enu2} mice greater than 6 weeks of age are utilized in experiments. C57B16 mice are obtained from Inotiv (C57BL/6NHsd). Male C57B16 mice greater than 6 weeks of age are utilized in experiments. All studies are conducted under a protocol approved by the Institutional Animal Care and Use Committee (IACUC).

On the day of the experiment (0 hour), mice are individually identified, weighed, and assigned to one of the treatment groups. Treatment groups have similar average body weights. Mice are administered with the compounds disclosed herein via oral gavage (10 mL/kg) and placed in metabolic cages to enable urine collection over 8 hours. Mice in metabolic cages have free access to gel diet (Bio-serve) and water for the duration of each study. The urine collection tubes are chilled with ice packs during the collection period to ensure stability of analytes in the urine. Urine volume is determined by weight and stored at −80° C. until use. For some test compounds, extra doses were administered during the 8-hour study period. At the end of each study, mice are euthanized using CO₂, and blood collected via cardiac puncture followed with a cervical dislocation to ensure death. Plasma is stored at −80° C. until use.

Urine samples are analyzed for creatinine by an assay kit (R&D Systems, KGE005) and amino acid levels by LC/MS. Plasma samples are used for measuring test compound concentrations and plasma phenylalanine by LC/MS at Maze Therapeutics.

All publication, patent applications, patents, and other references mentioned herein are expressly incorporated by reference in their entireties, to the same extent as if each were incorporated by reference individually.

1. A compound of formula (I-H): 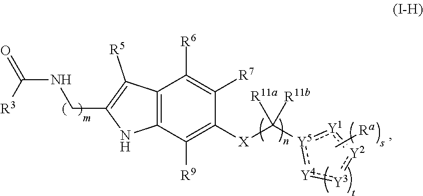
or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R³ is C₁₋₆alkyl, C₁₋₆haloalkyl, C₁₋₆alkoxy, —C(O)C₁₋₆alkyl, C₃₋₁₀cycloalkyl, —N(R⁴)₂, 5-20 membered heteroaryl, —C₁₋₆alkyl(5-20 membered heteroaryl), or 4-10 membered heterocyclyl, wherein each R³ is optionally substituted with one or more R^{a}, and wherein when R³ is 6-10 membered heteroaryl, the 6-10 membered heteroaryl is unsubstituted;
each R⁴ is independently H, C₁₋₆alkyl, C₁₋₆haloalkyl, —C₁₋₆alkyl(5-20 membered heteroaryl), or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₁₋₆thioalkyl, C₃₋₁₀cycloalkyl, C₁₋₆haloalkyl, or 5-20 membered heteroaryl, wherein
the C₁₋₆alkyl, C₁₋₆alkoxy, or C₃₋₁₀cycloalkyl of R⁵ and R⁷ are each independently optionally substituted with one or more halo or CN;
R⁶ and R⁹ are each independently H or halo;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently D, —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl of R^{a} is optionally substituted with one or more R^{b};
each R^{b} is independently D, —OH, halo, C₁₋₆alkyl, or C₁₋₆alkoxy
Y¹, Y², Y³, Y⁴ and Y⁵ are each independently —C—, —CH—, —CH₂—, —N—, —NH—, —S—, or —O—;
s is an integer from 0-6;
t is 1 or 2; and [symbol]
represents a single or double bond.

2-9. (canceled)

10. The compound of claim 1, or a stereoisomer or tautomer thereof, or a pharmaceutically acceptable salt of any of the foregoing, wherein m is 1 and n is 1.

11. The compound of claim 1, or a stereoisomer or tautomer thereof, or a pharmaceutically acceptable salt of any of the foregoing, wherein m is 1 and n is 0.

12-13. (canceled)

14. The compound of claim 1, or a stereoisomer or tautomer thereof, or a pharmaceutically acceptable salt of any of the foregoing, wherein R³ is methyl, ethyl, or isopropyl.

15. (canceled)

16. The compound of claim 1, or a stereoisomer or tautomer thereof, or a pharmaceutically acceptable salt of any of the foregoing, wherein R³ is selected from the group consisting of

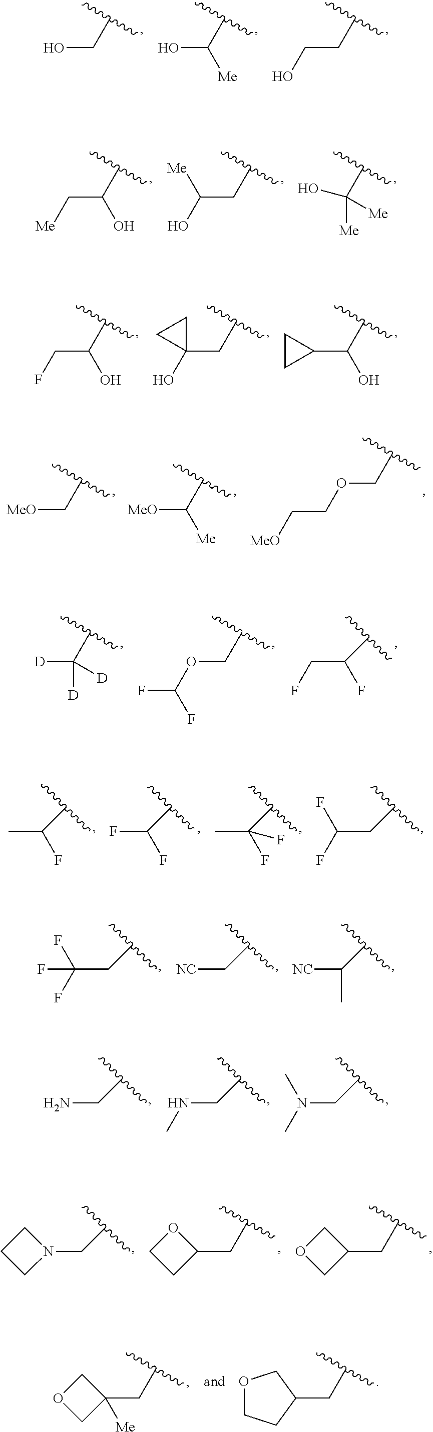

17-18. (canceled)

19. The compound of claim 1, or a stereoisomer or tautomer thereof, or a pharmaceutically acceptable salt of any of the foregoing, wherein R³ is selected from the group consisting of

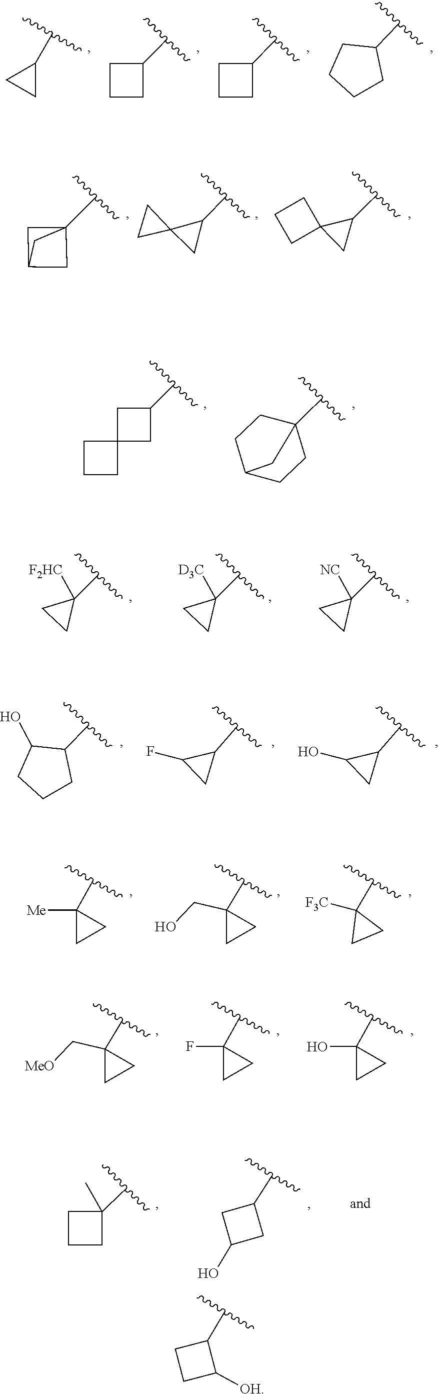

20-24. (canceled)

25. The compound of claim 1, or a stereoisomer or tautomer thereof, or a pharmaceutically acceptable salt of any of the foregoing, wherein R³ is selected from the group consisting of

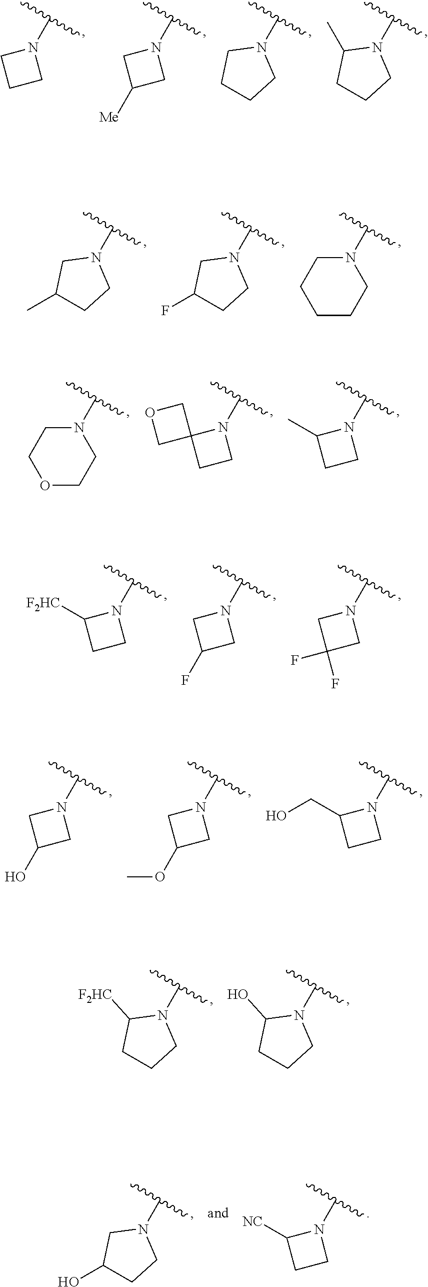

26. (canceled)

27. The compound of claim 1, or a stereoisomer or tautomer thereof, or a pharmaceutically acceptable salt of any of the foregoing, wherein R³ is selected from the group consisting of

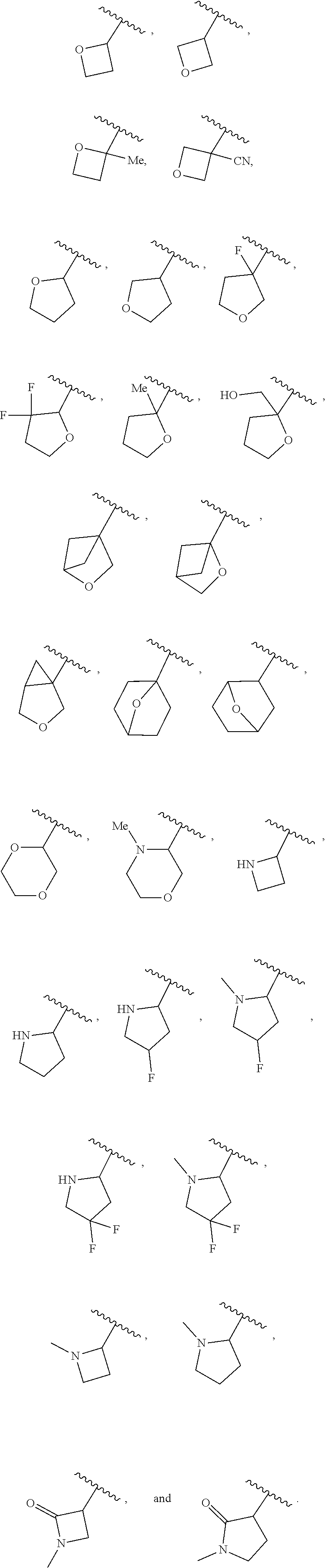

28. (canceled)

29. The compound of claim 1, or a stereoisomer or tautomer thereof, or a pharmaceutically acceptable salt of any of the foregoing, wherein R³ is

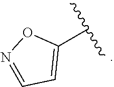 [SMILES: *c1ccno1]

30-51. (canceled)

52. The compound of claim 1, or a stereoisomer or tautomer thereof, or a pharmaceutically acceptable salt of any of the foregoing, wherein X is —O—.

53. The compound of claim 1, or a stereoisomer or tautomer thereof, or a pharmaceutically acceptable salt of any of the foregoing, wherein X is —S—.

54-55. (canceled)

56. The compound of claim 1, or a stereoisomer or tautomer thereof, or a pharmaceutically acceptable salt of any of the foregoing, wherein X is —CH₂—, or —CH(C₁₋₆alkyl)-.

57. (canceled)

58. The compound of claim 1, or a stereoisomer or tautomer thereof, or a pharmaceutically acceptable salt of any of the foregoing, wherein X is —NH—.

59. The compound of claim 1, wherein, n is 1, one of R^{12a} and R^{12b} is H, or C₁₋₆alkyl, and the other of R^{12a} and R^{12b} is taken together with R^{11a} or R^{11b} to form a C₃₋₁₀cycloalkyl.

60-62. (canceled)

63. The compound of claim 1, or a stereoisomer or tautomer thereof, or a pharmaceutically acceptable salt of any of the foregoing, wherein the compound is of formula:

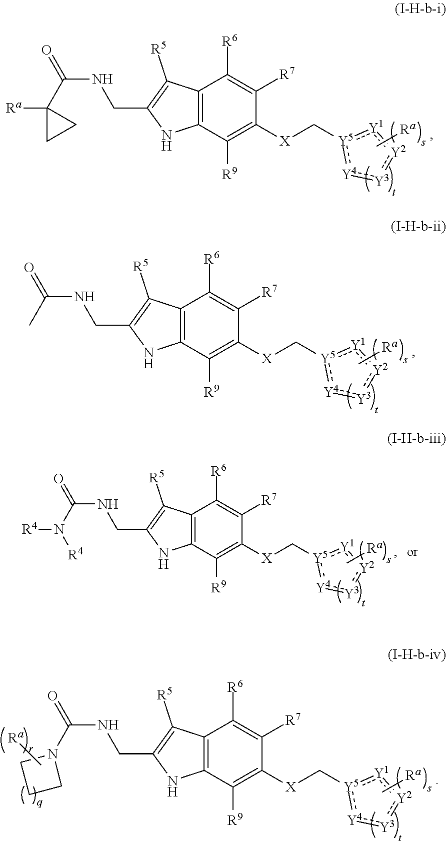

64. The compound of claim 1, or a stereoisomer or tautomer thereof, or a pharmaceutically acceptable salt of any of the foregoing, wherein the compound, is selected from the group consisting of 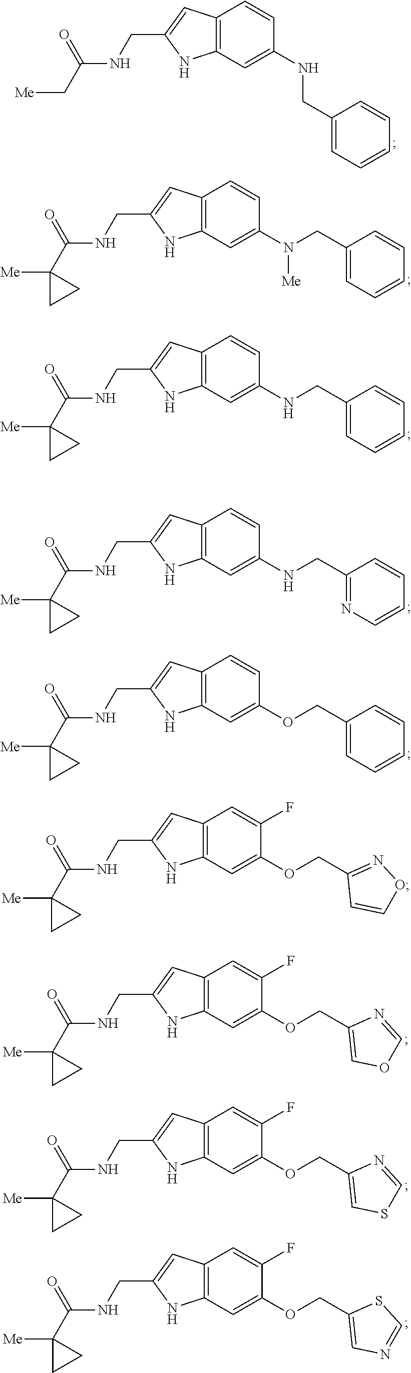 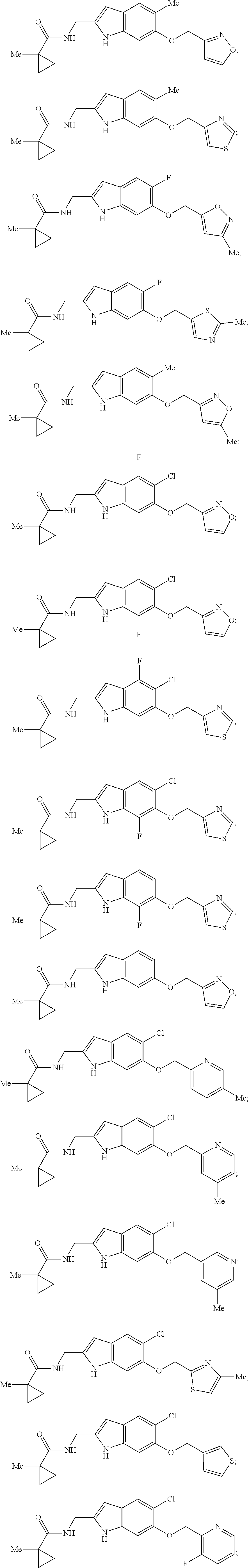 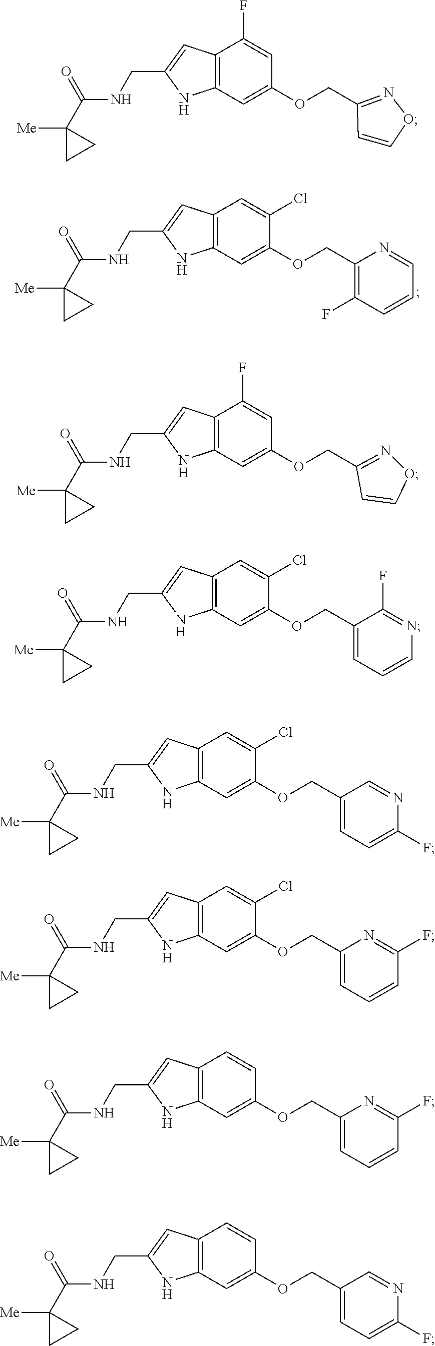 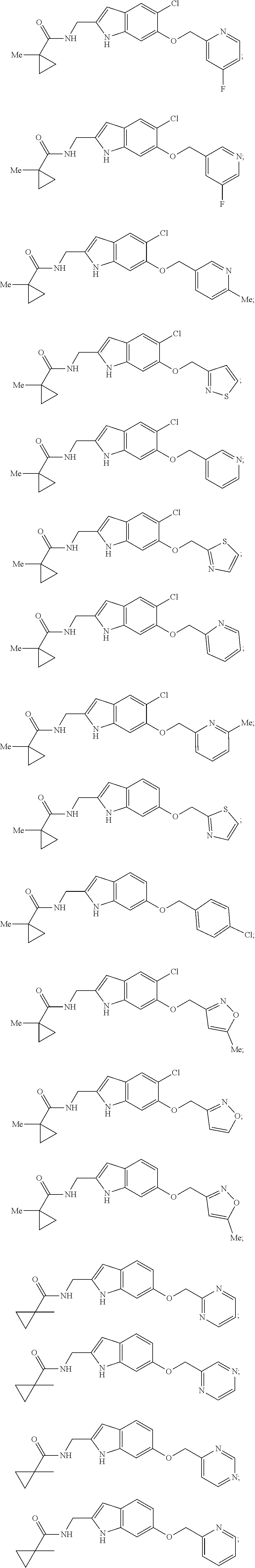 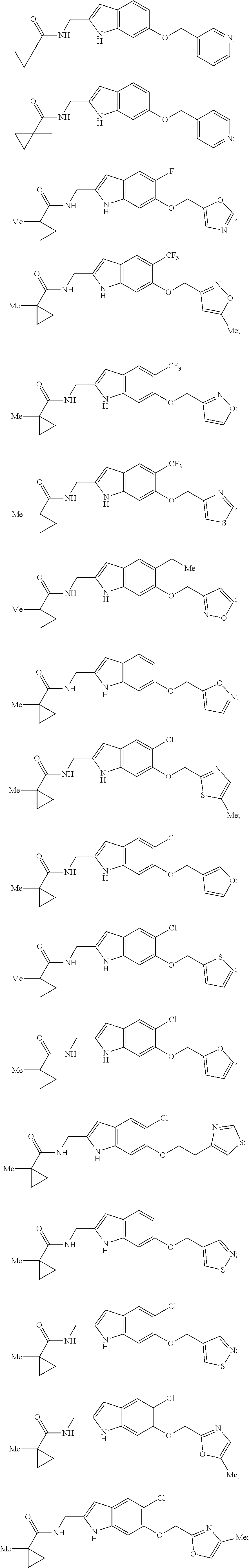 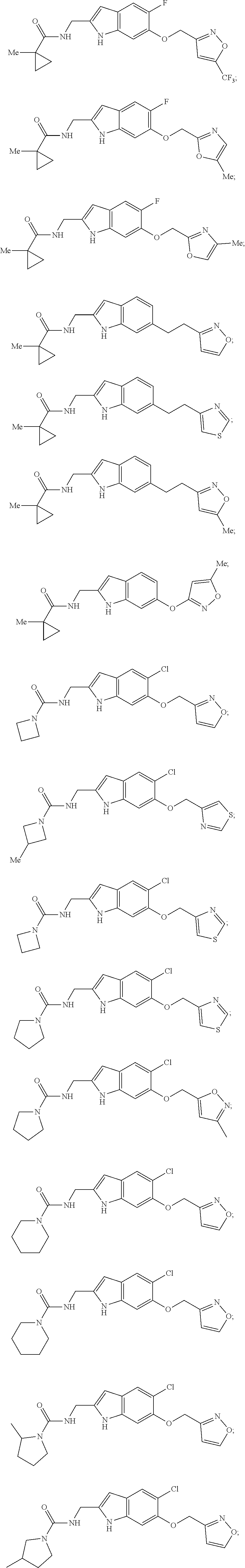 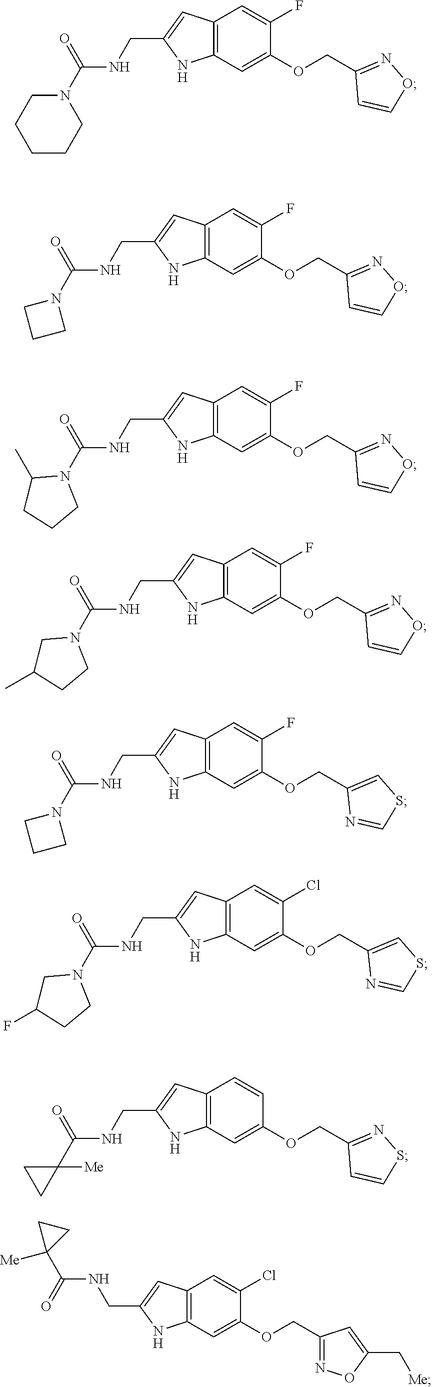 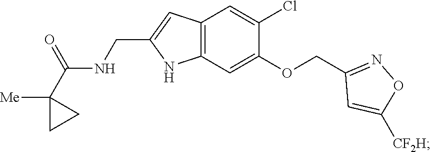 [SMILES: CC1(C(=O)NCc2cc3cc(Cl)c(OCc4cc(C(F)F)on4)cc3[nH]2)CC1] 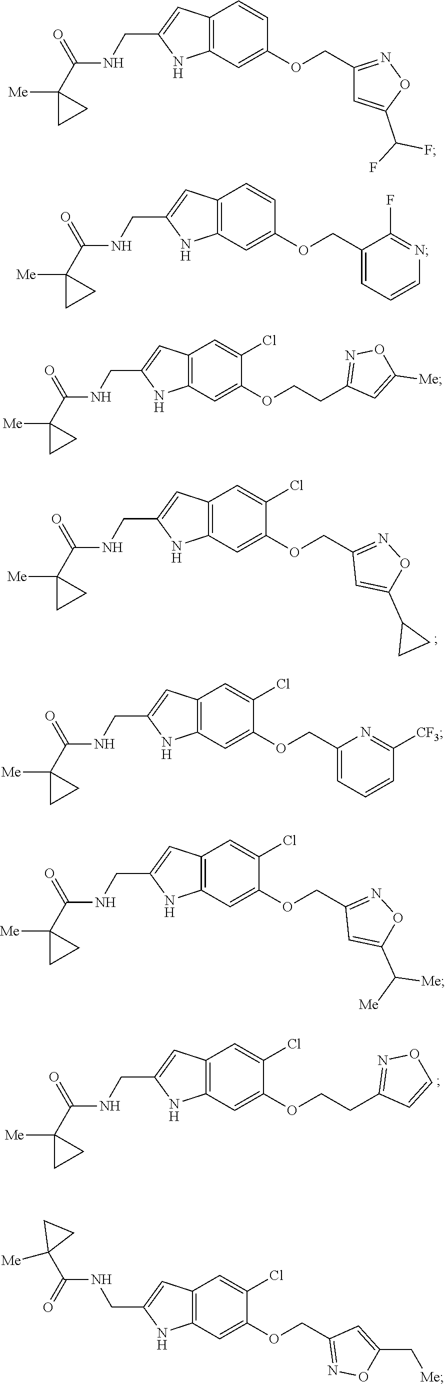 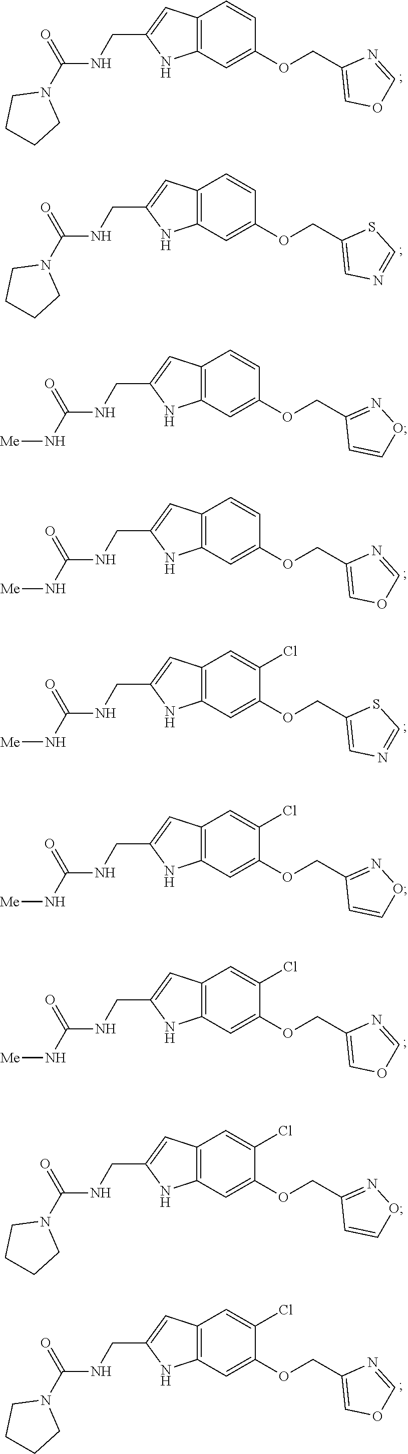 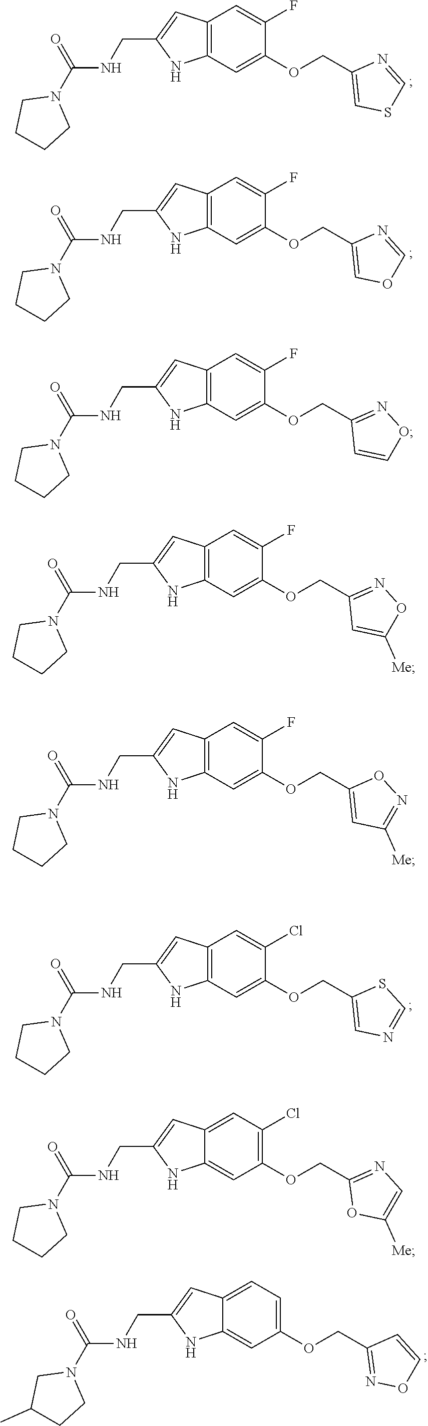 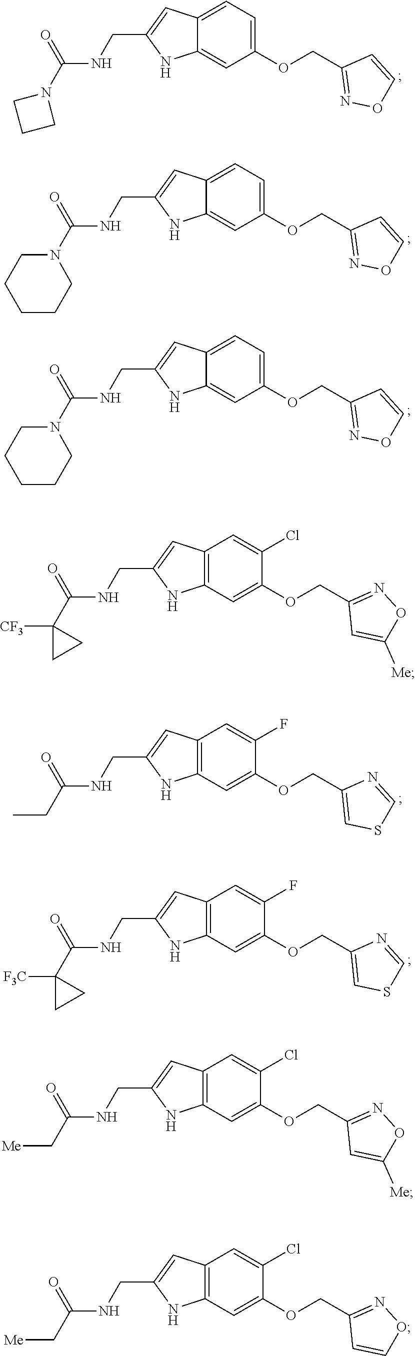 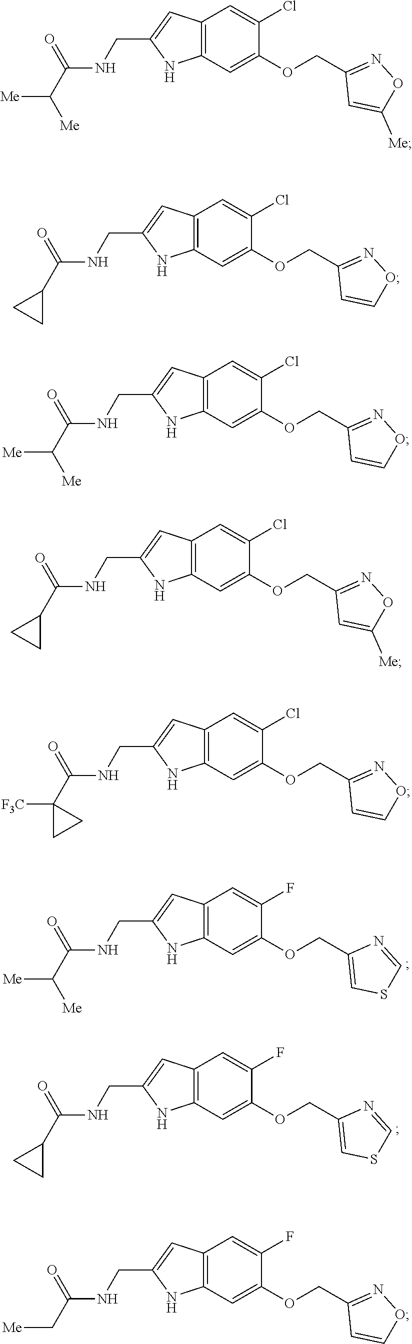 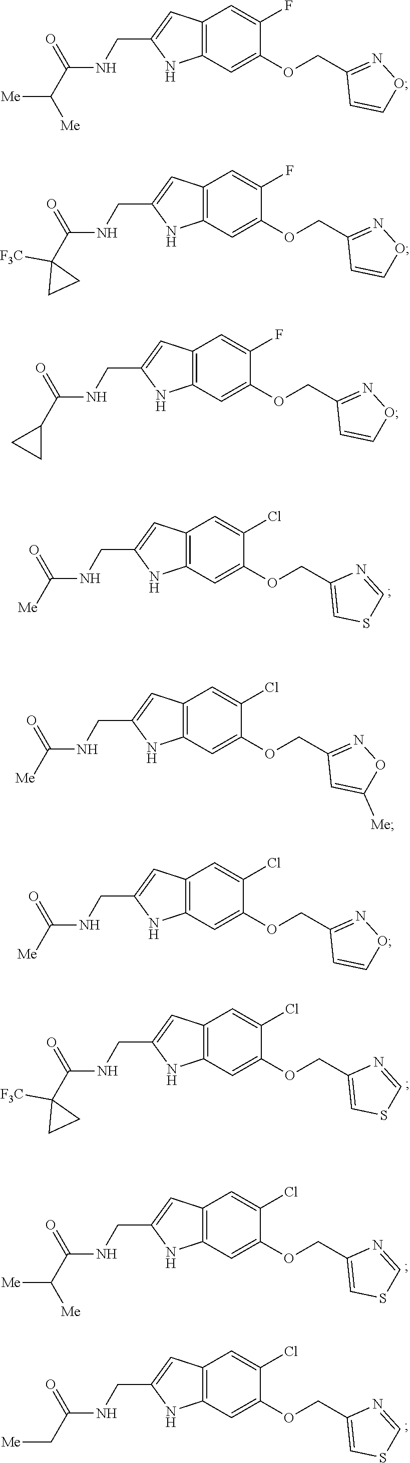 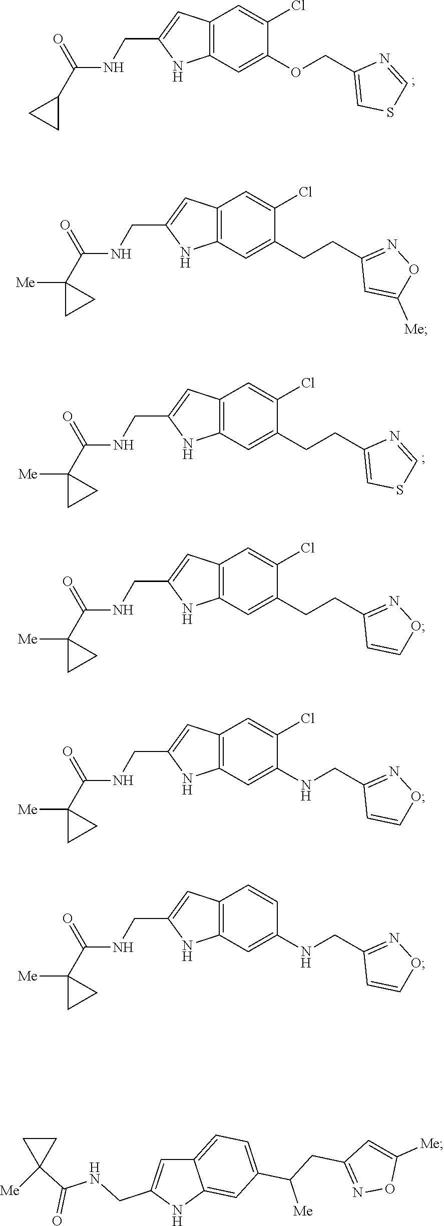 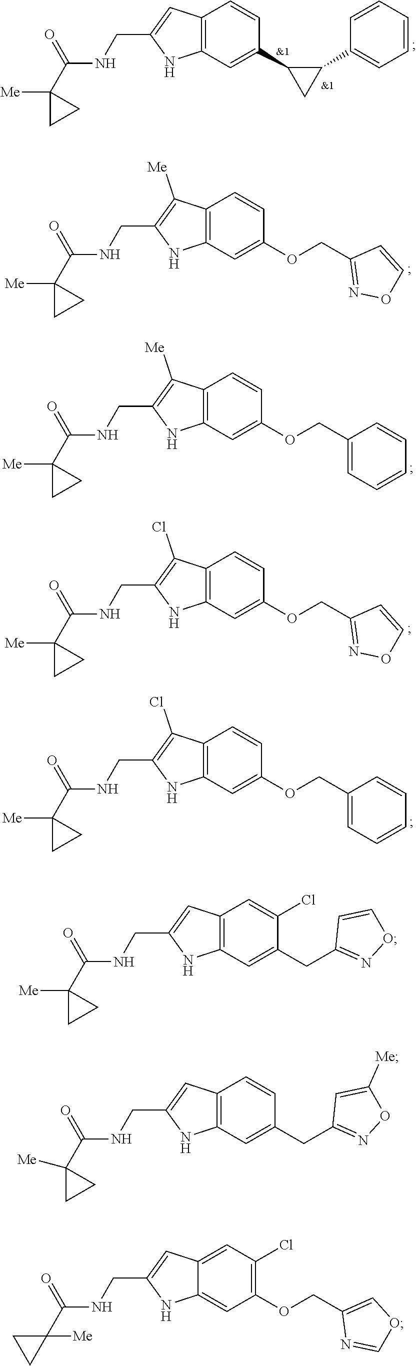 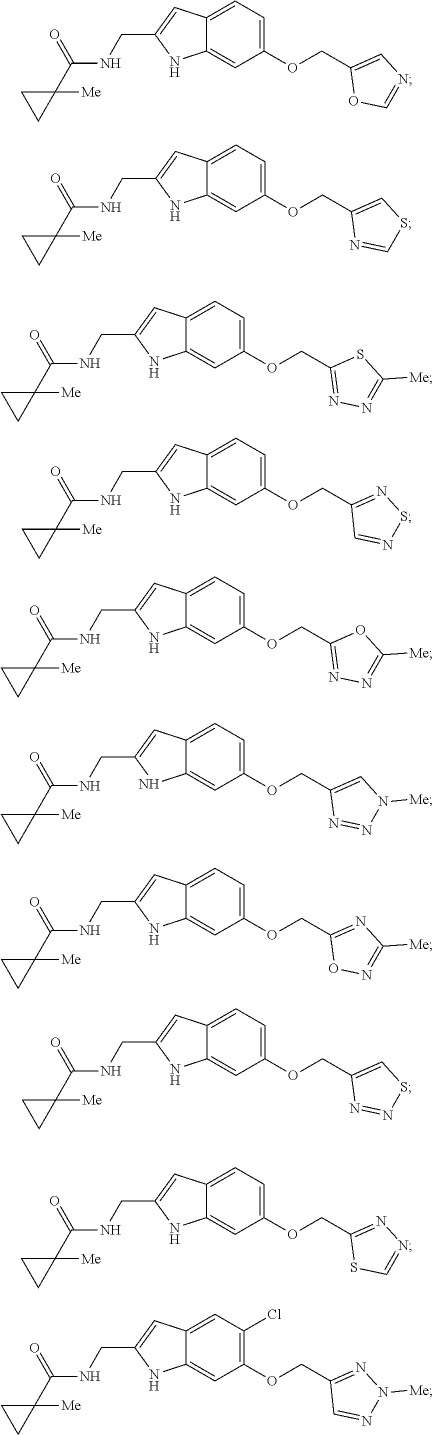 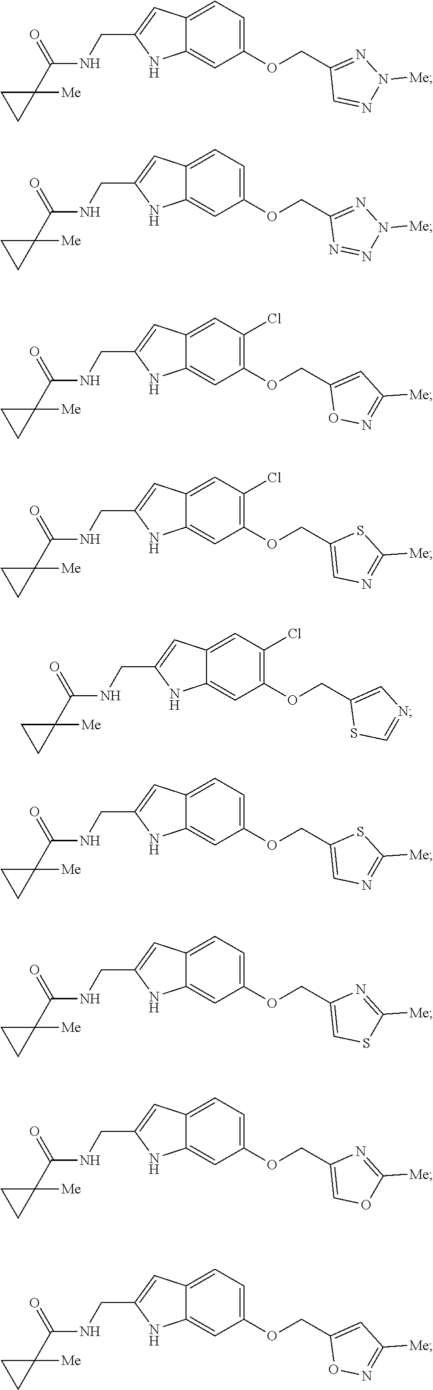 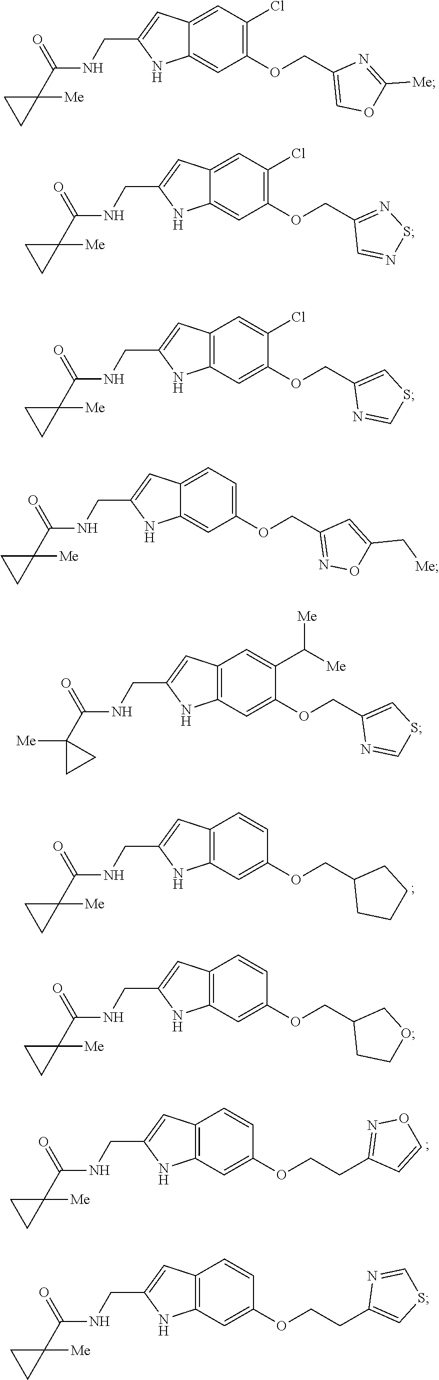 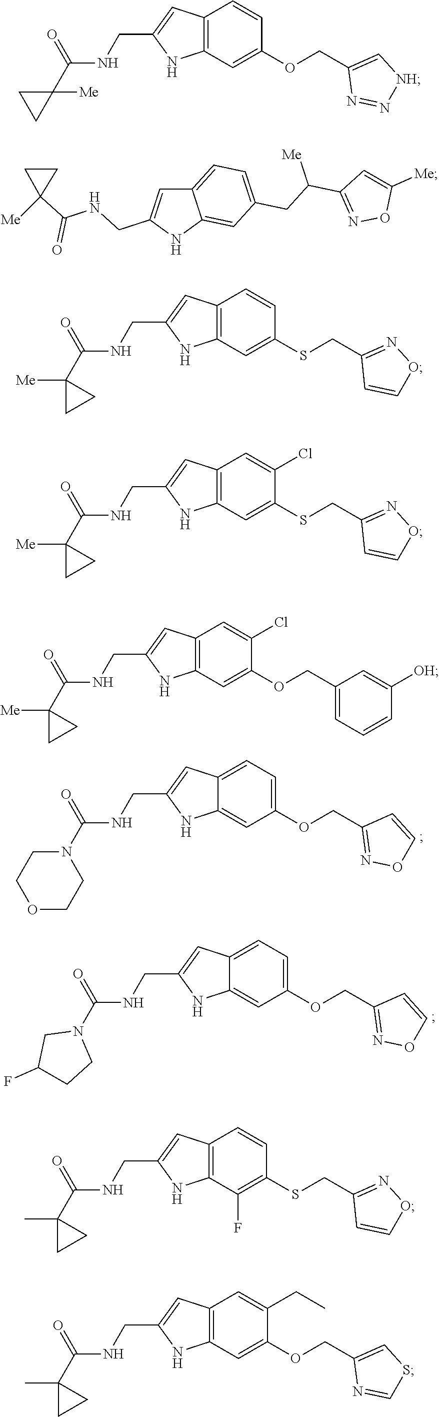 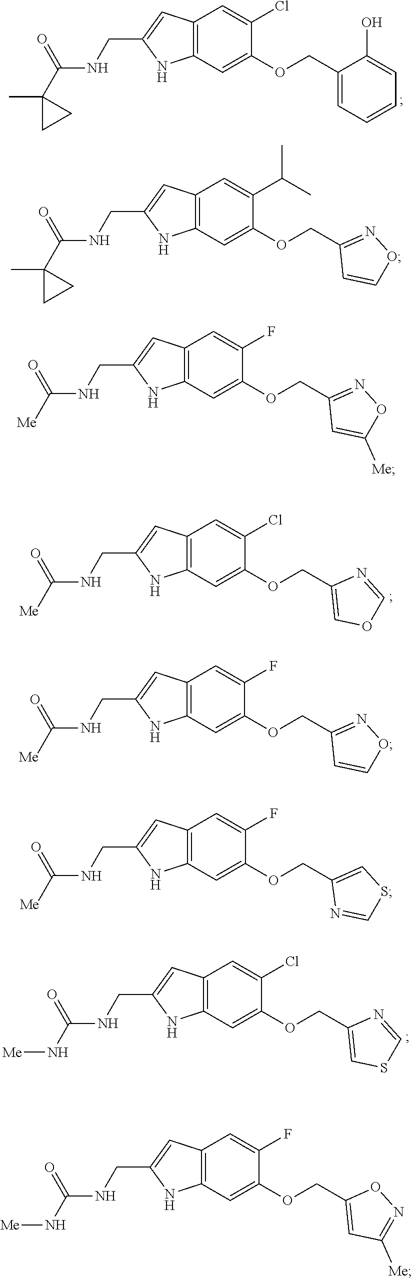 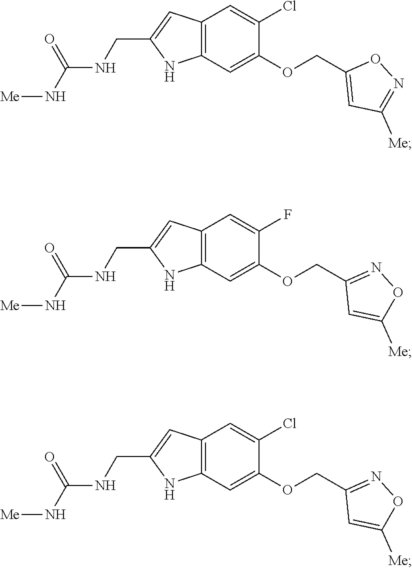 [SMILES: CNC(=O)NCc1cc2cc(Cl)c(OCc3cc(C)no3)cc2[nH]1 ; CNC(=O)NCc1cc2cc(F)c(OCc3cc(C)on3)cc2[nH]1 ; CNC(=O)NCc1cc2cc(Cl)c(OCc3cc(C)on3)cc2[nH]1] 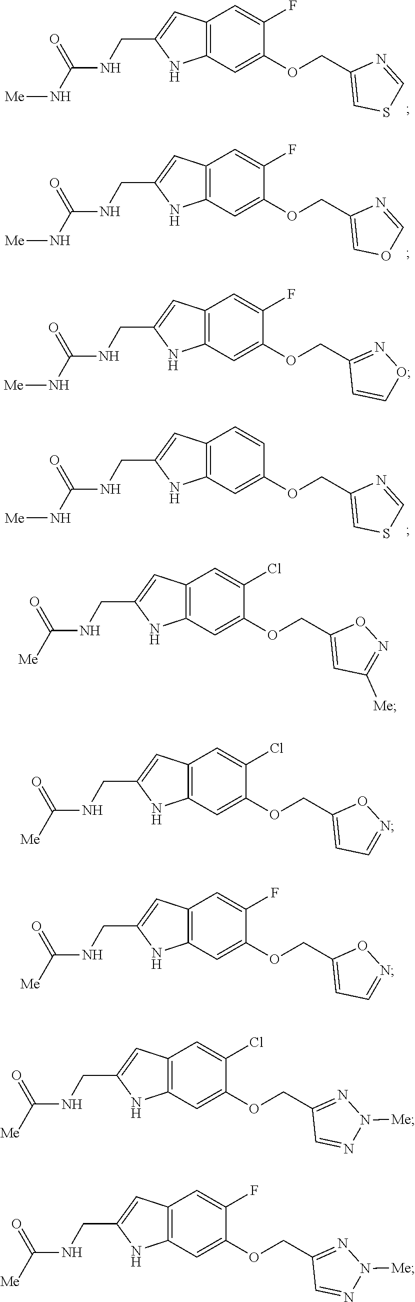 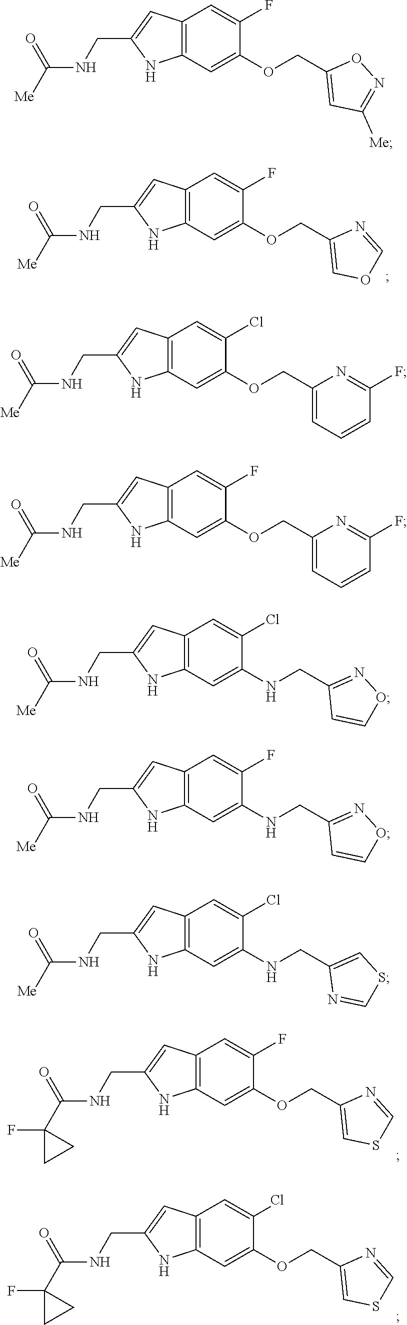 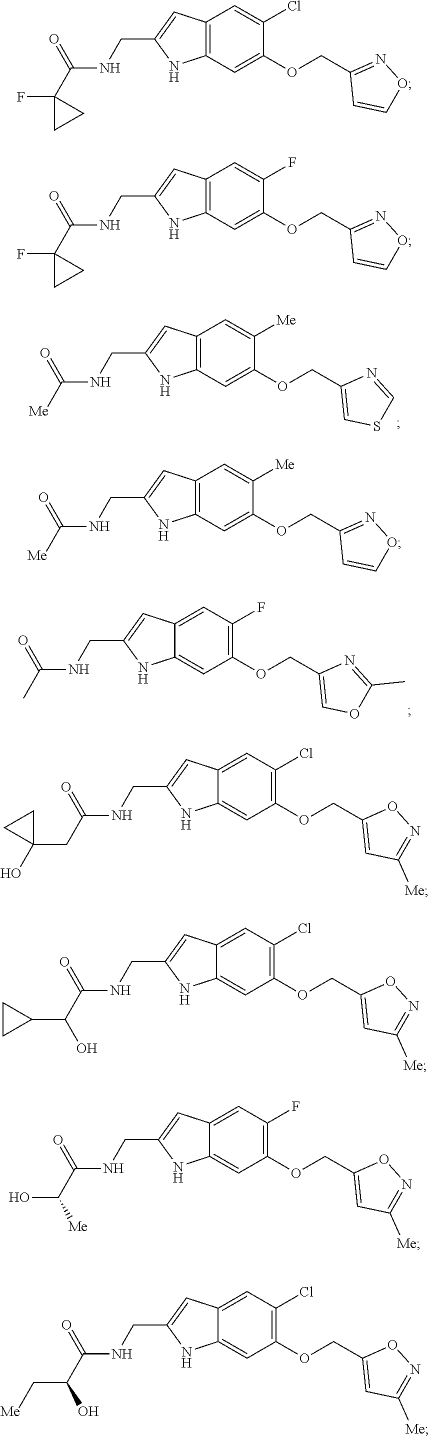 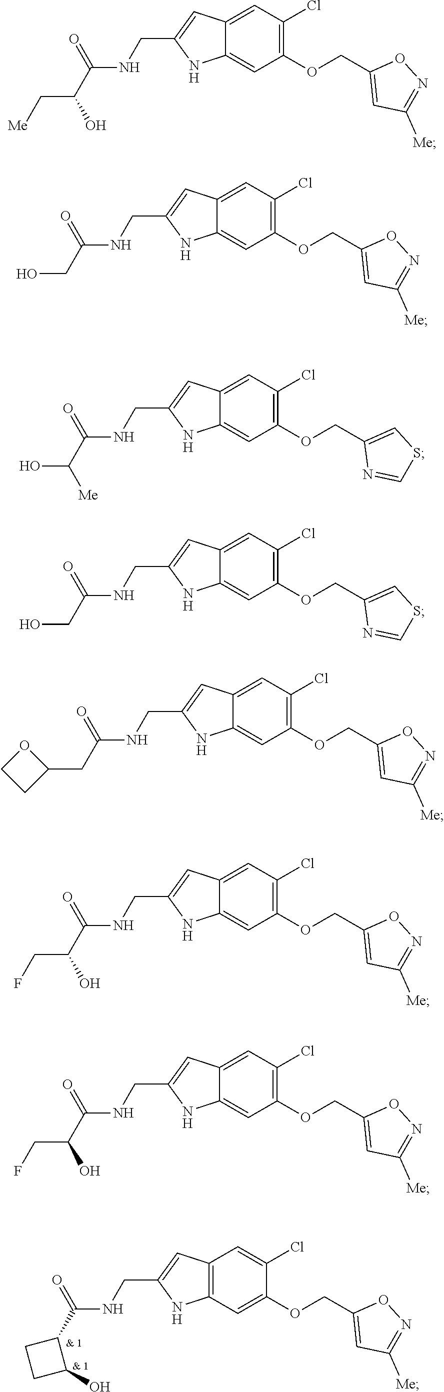 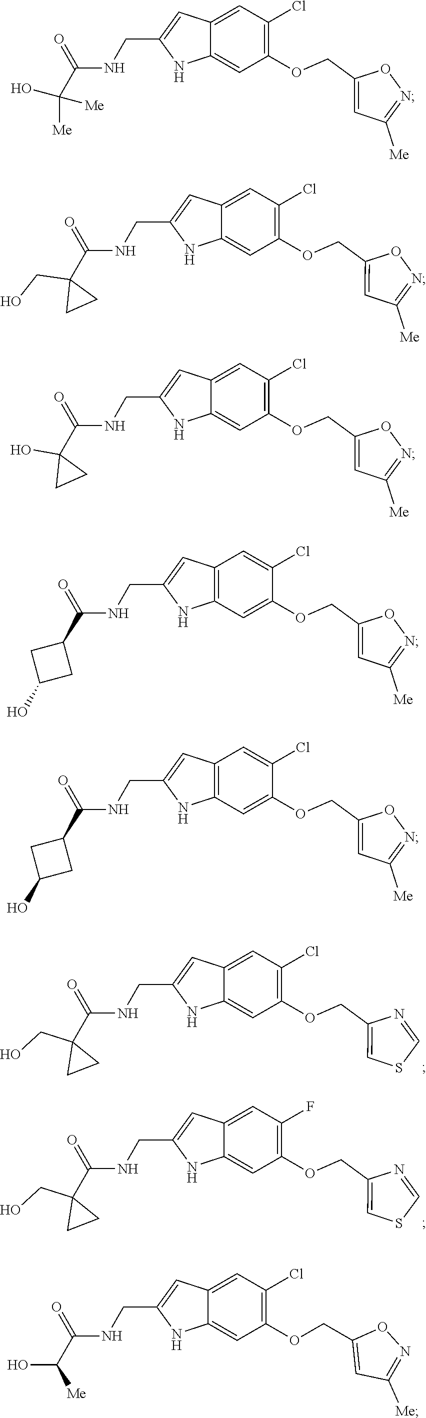 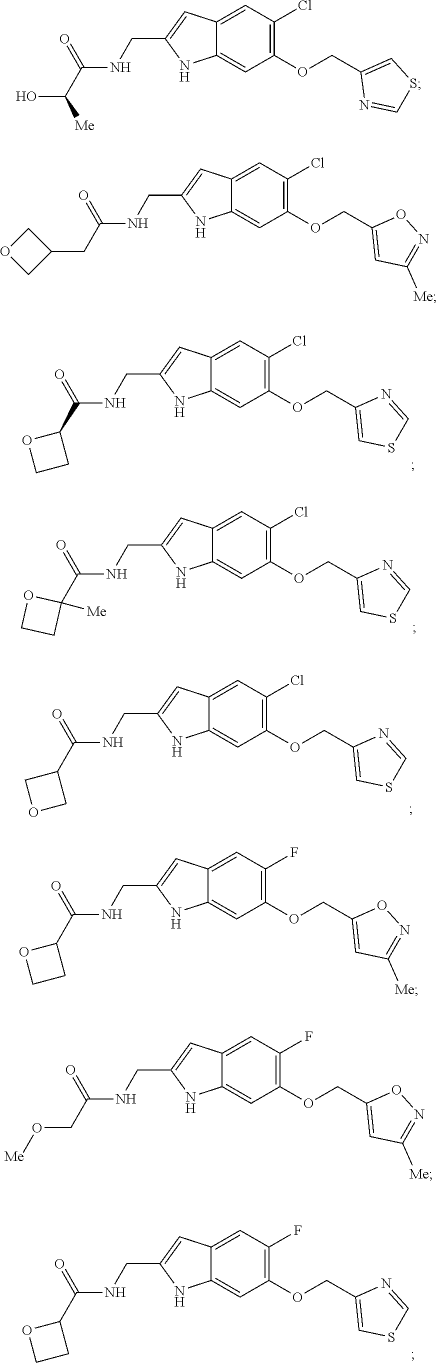 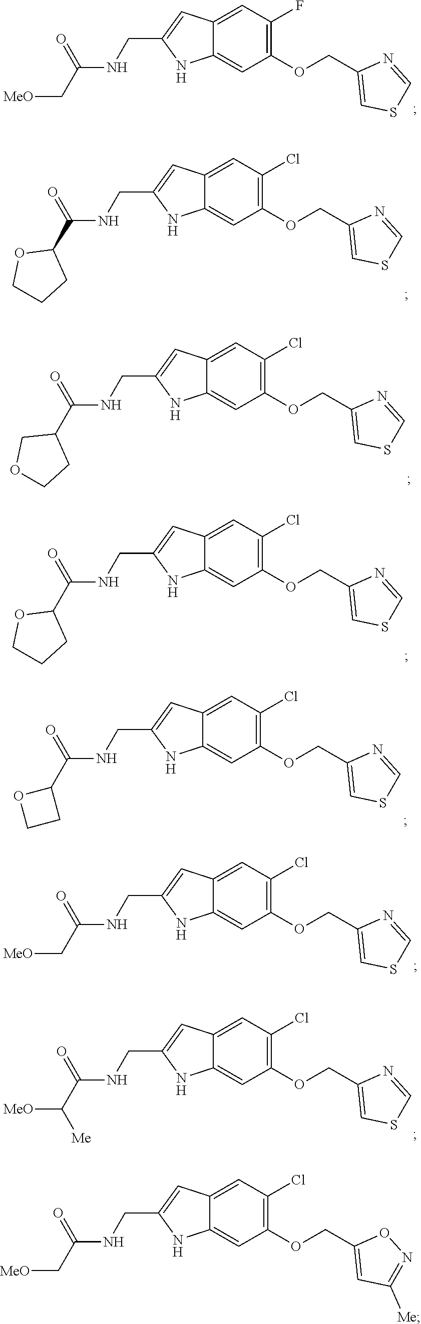 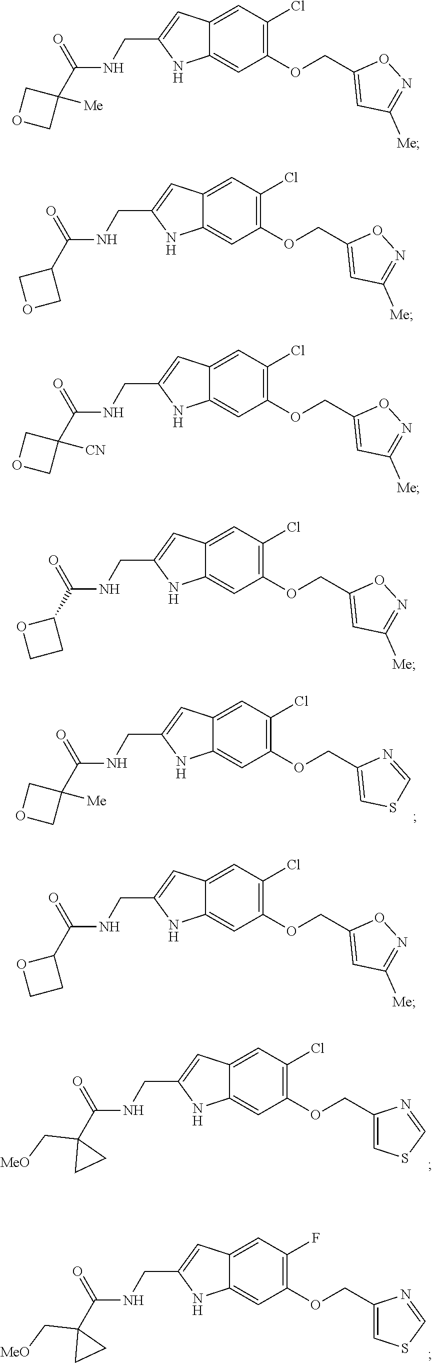 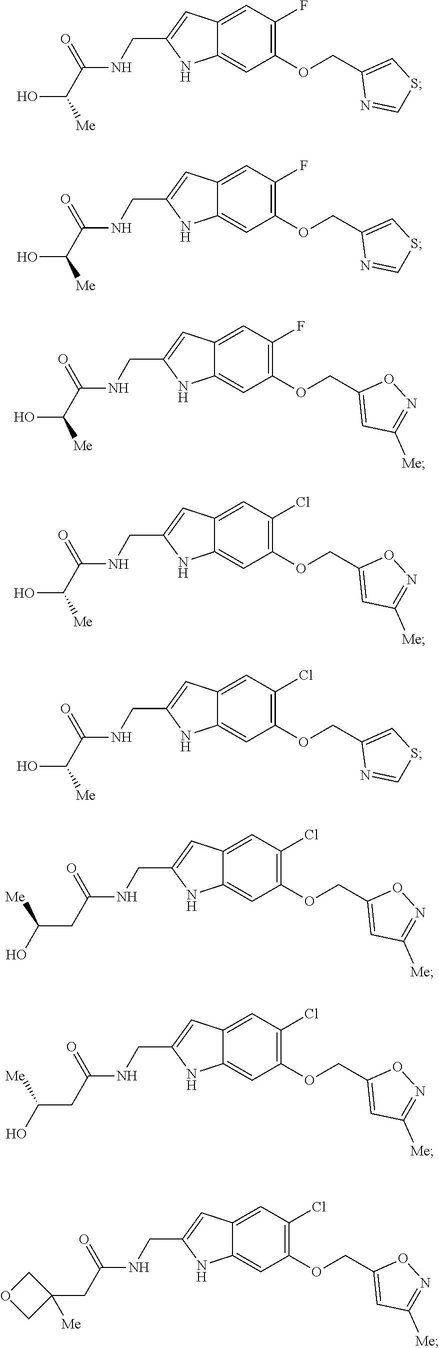 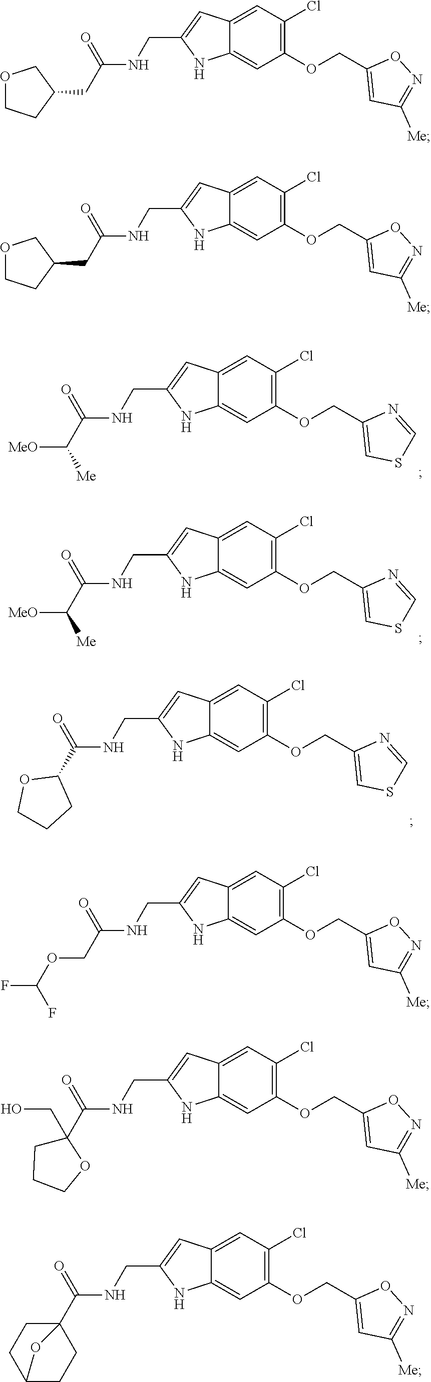 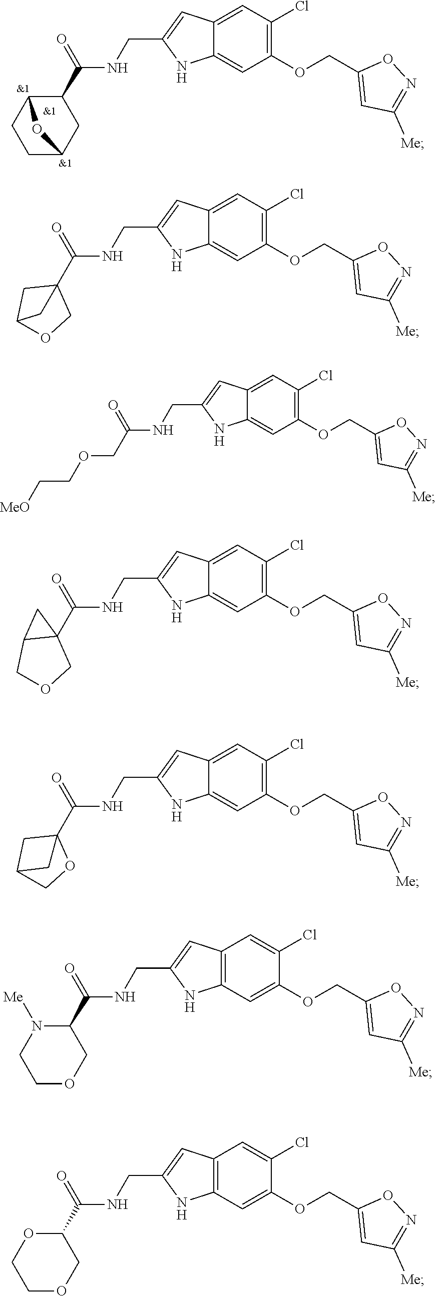 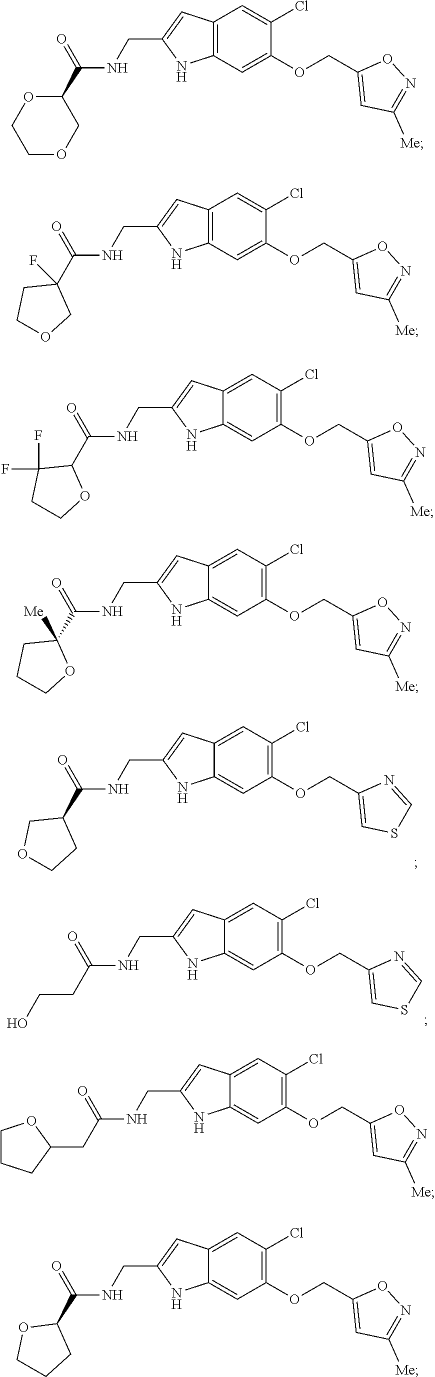 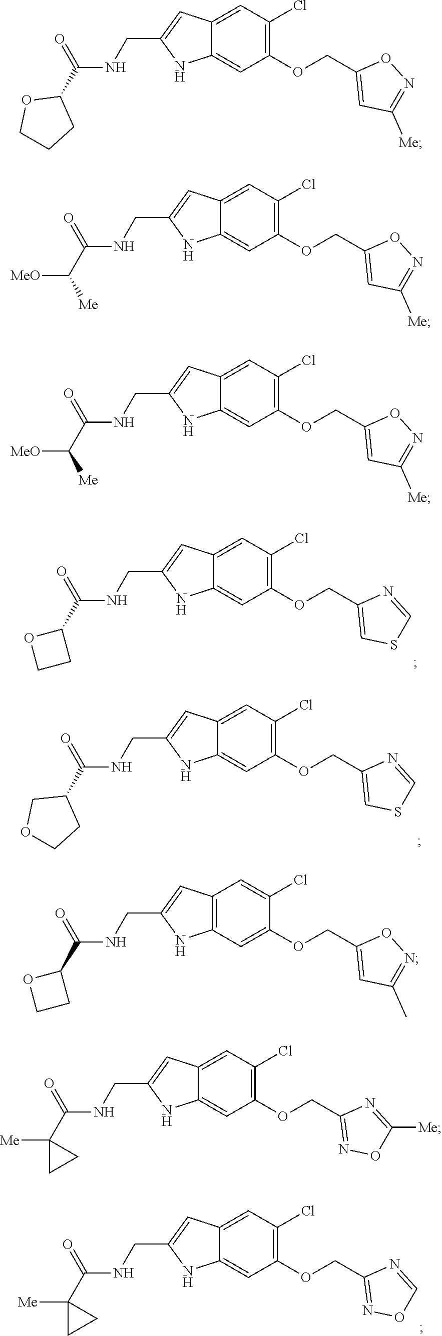 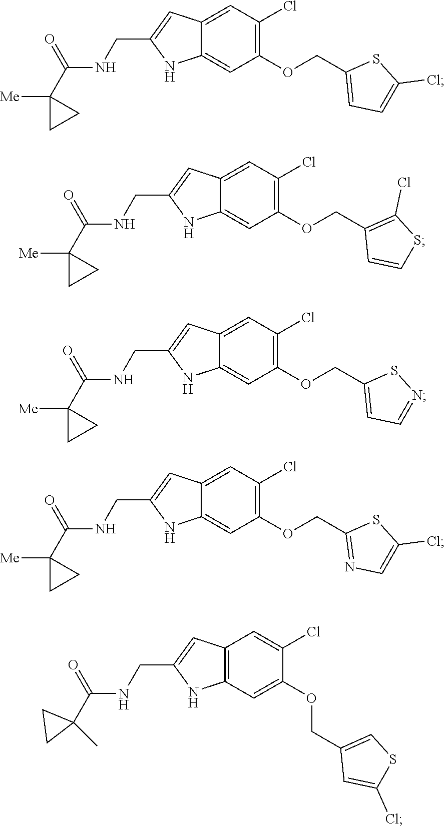 [SMILES: CC1(C(=O)NCc2cc3cc(Cl)c(OCc4ccc(Cl)s4)cc3[nH]2)CC1 ; CC1(C(=O)NCc2cc3cc(Cl)c(OCc4ccsc4Cl)cc3[nH]2)CC1 ; CC1(C(=O)NCc2cc3cc(Cl)c(OCc4ccns4)cc3[nH]2)CC1 ; CC1(C(=O)NCc2cc3cc(Cl)c(OCc4ncc(Cl)s4)cc3[nH]2)CC1 ; CC1(C(=O)NCc2cc3cc(Cl)c(OCc4csc(Cl)c4)cc3[nH]2)CC1] 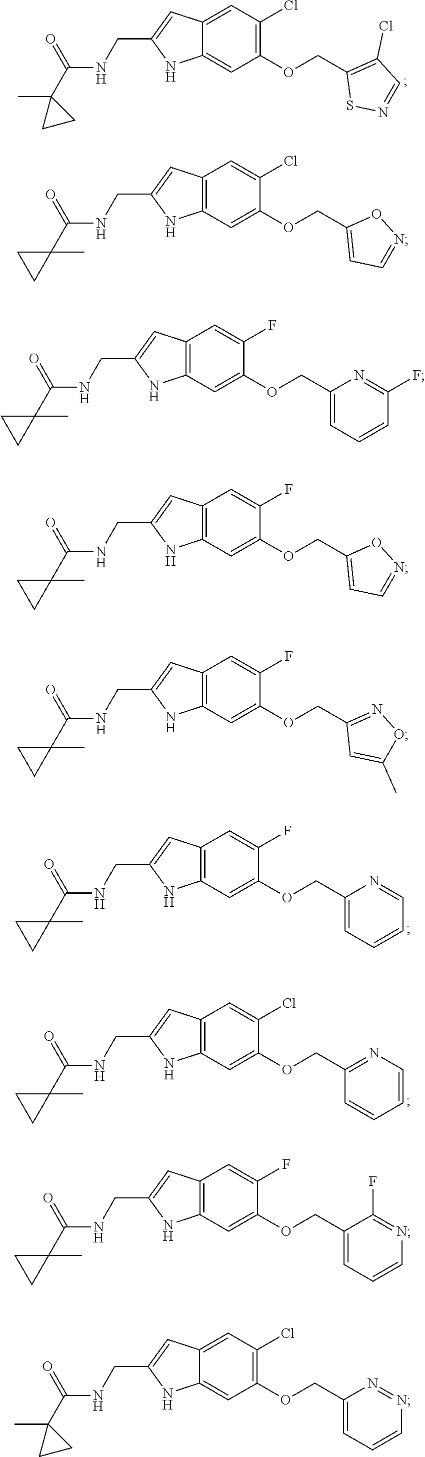 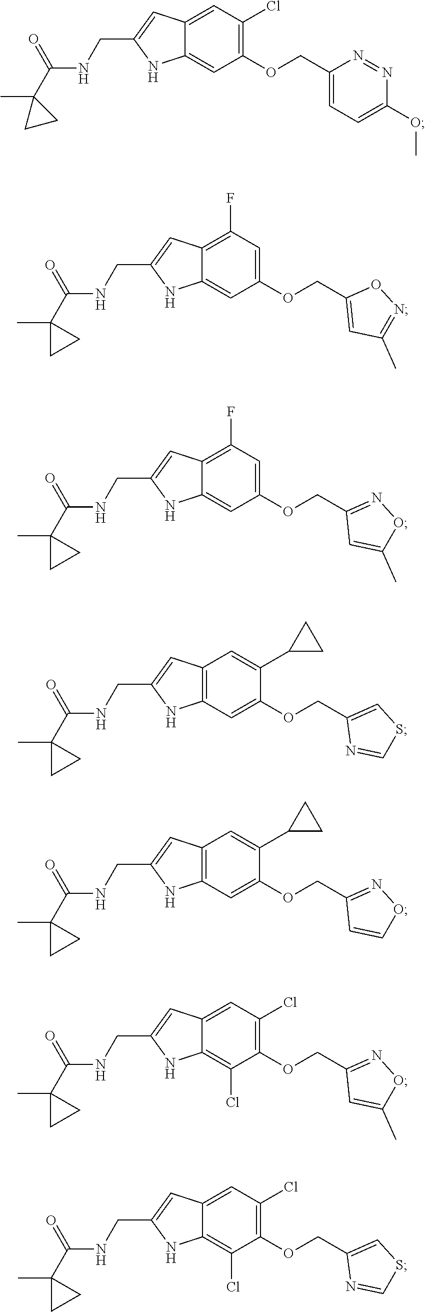 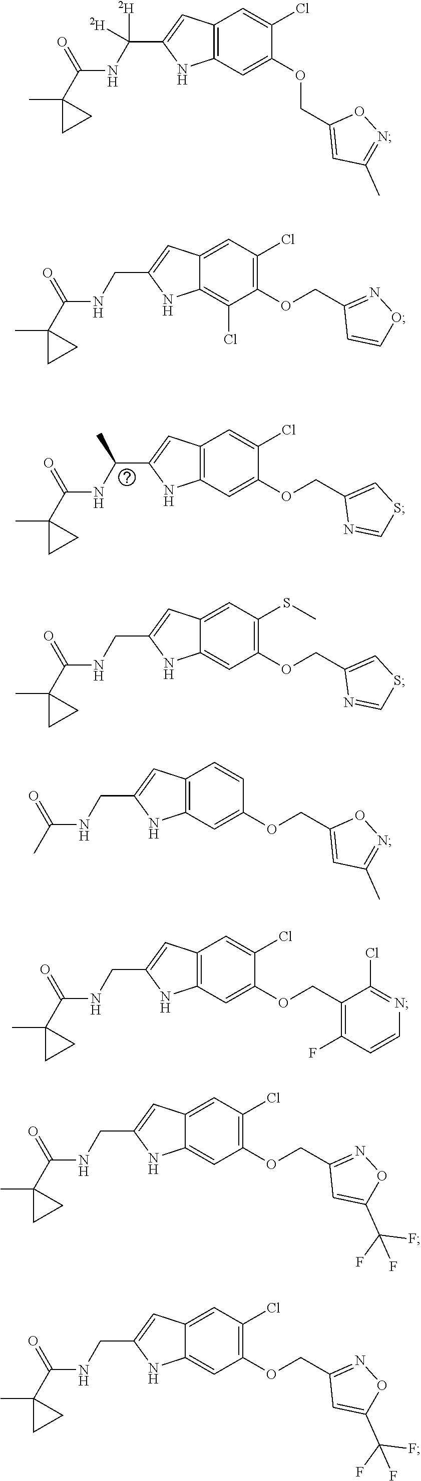 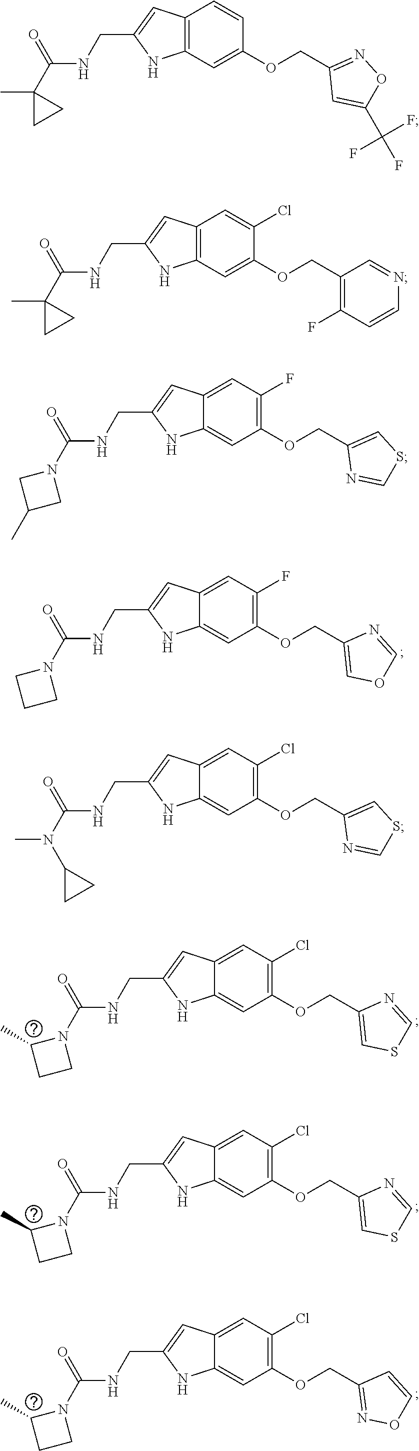 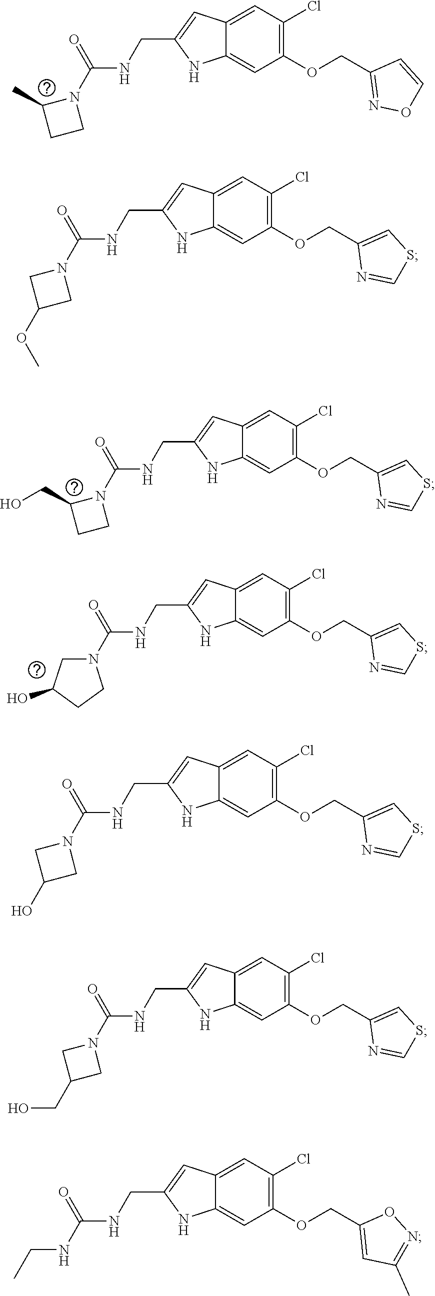 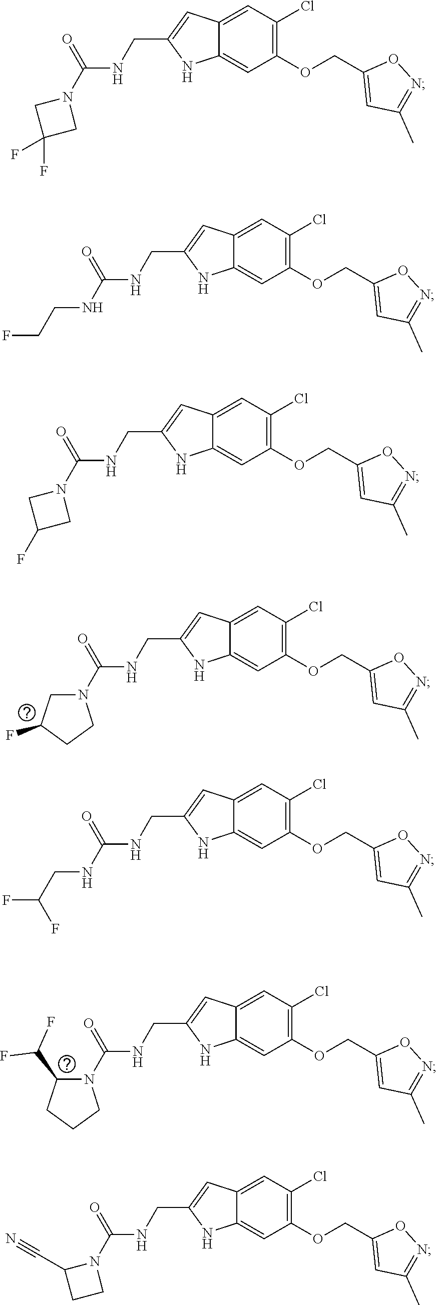 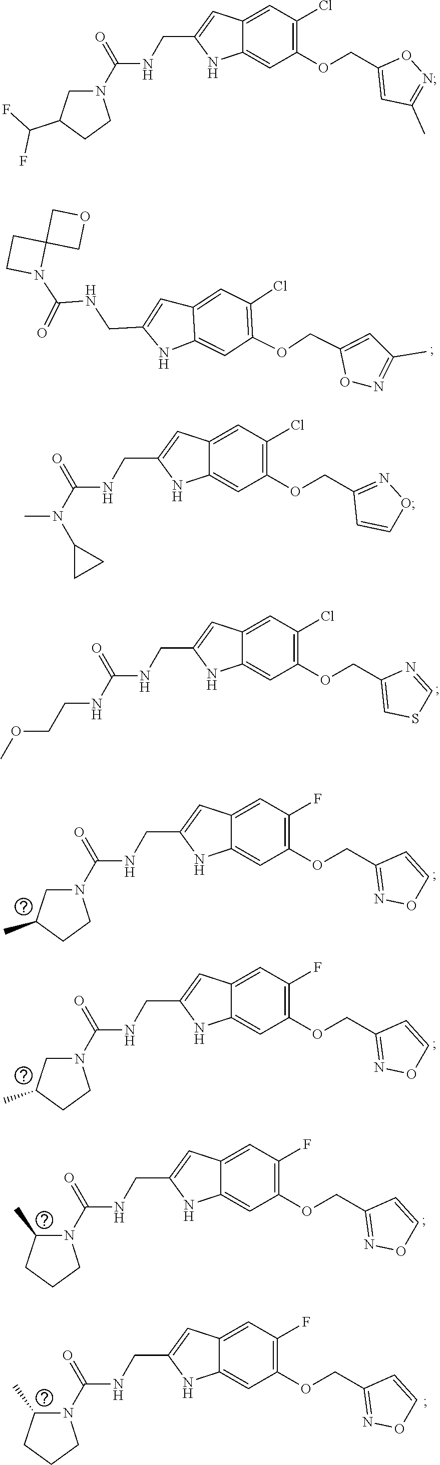 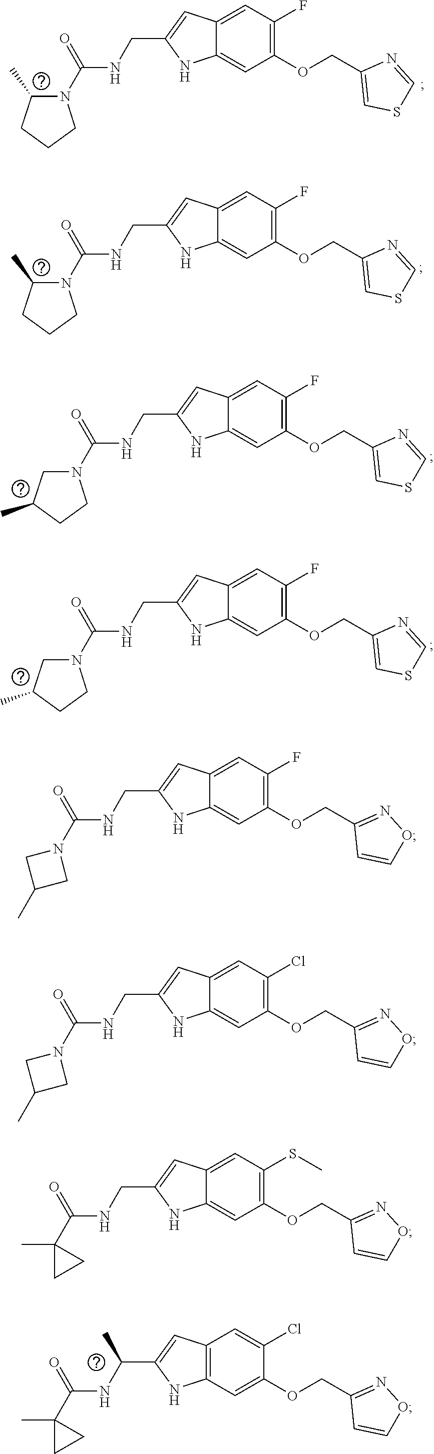 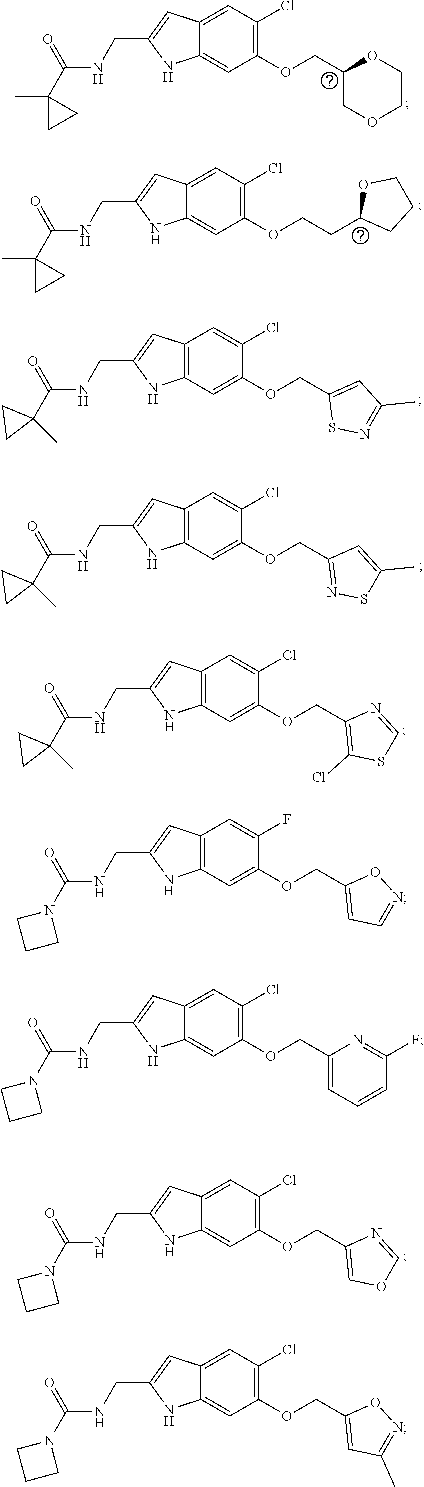 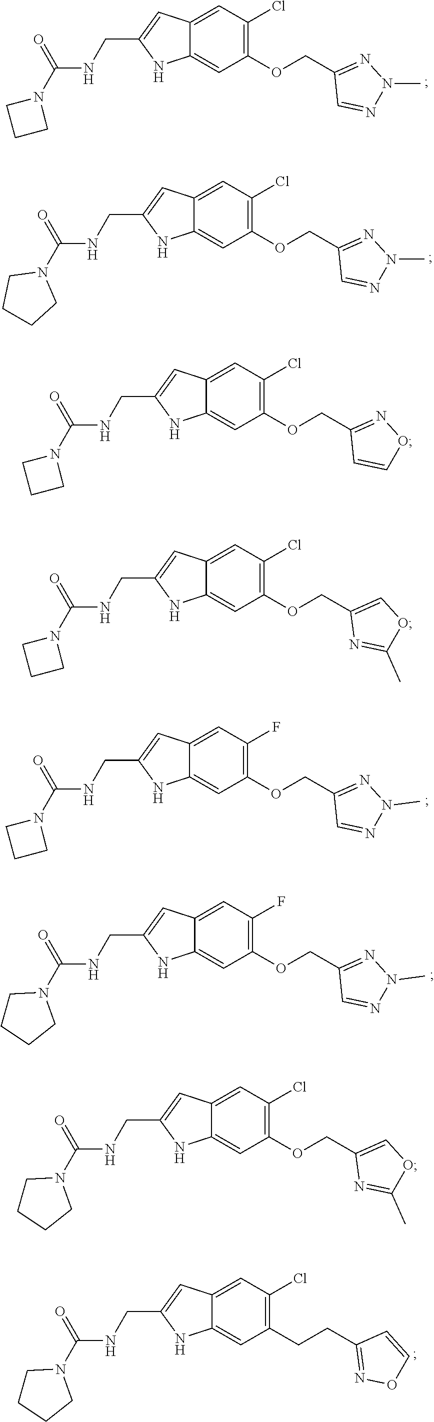 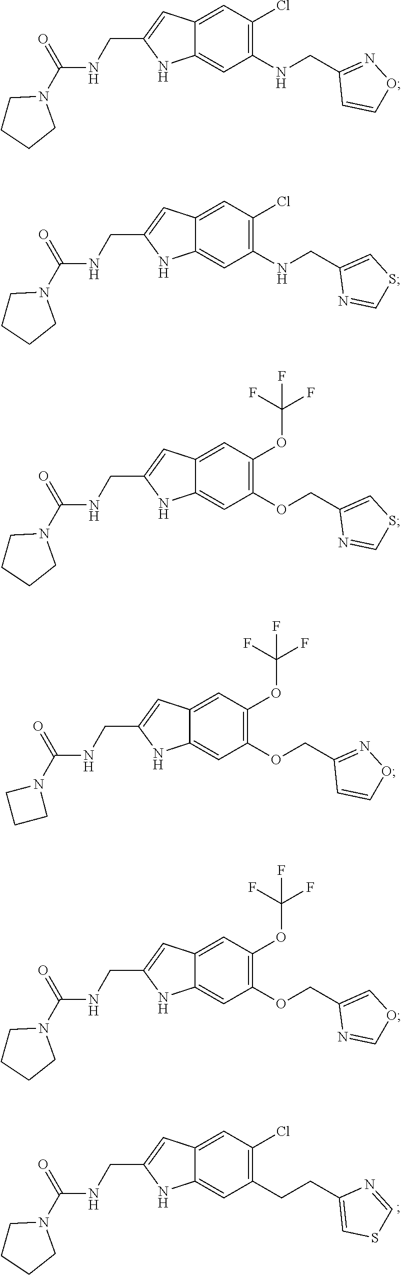 [SMILES: O=C(NCc1cc2cc(Cl)c(NCc3ccon3)cc2[nH]1)N1CCCC1 ; O=C(NCc1cc2cc(Cl)c(NCc3cscn3)cc2[nH]1)N1CCCC1 ; O=C(NCc1cc2cc(OC(F)(F)F)c(OCc3cscn3)cc2[nH]1)N1CCCC1 ; O=C(NCc1cc2cc(OC(F)(F)F)c(OCc3ccon3)cc2[nH]1)N1CCC1 ; O=C(NCc1cc2cc(OC(F)(F)F)c(OCc3cocn3)cc2[nH]1)N1CCCC1 ; O=C(NCc1cc2cc(Cl)c(CCc3cscn3)cc2[nH]1)N1CCCC1] 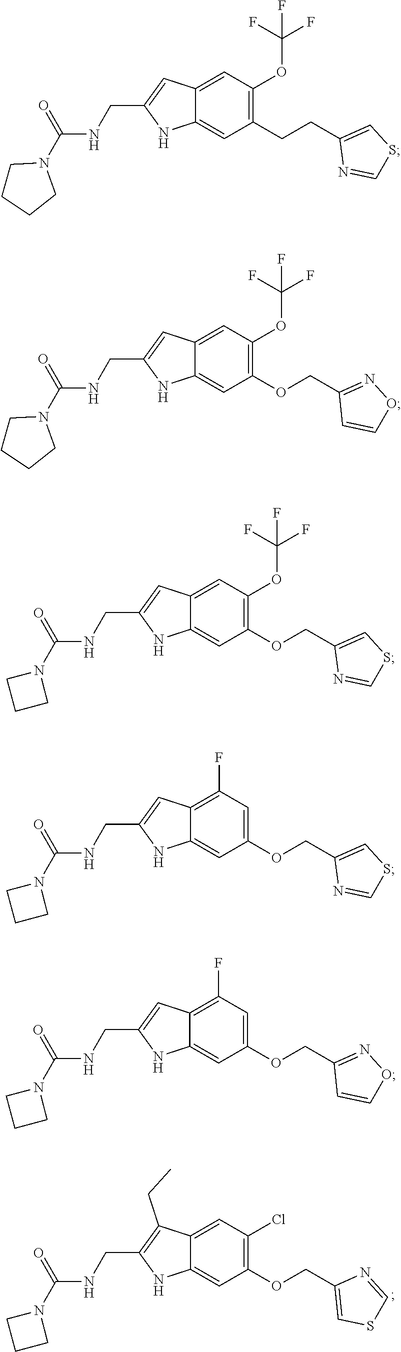 [SMILES: O=C(NCc1cc2cc(OC(F)(F)F)c(CCc3cscn3)cc2[nH]1)N1CCCC1 ; O=C(NCc1cc2cc(OC(F)(F)F)c(OCc3ccon3)cc2[nH]1)N1CCCC1 ; O=C(NCc1cc2cc(OC(F)(F)F)c(OCc3cscn3)cc2[nH]1)N1CCC1 ; O=C(NCc1cc2c(F)cc(OCc3cscn3)cc2[nH]1)N1CCC1 ; O=C(NCc1cc2c(F)cc(OCc3ccon3)cc2[nH]1)N1CCC1 ; CCc1c(CNC(=O)N2CCC2)[nH]c2cc(OCc3cscn3)c(Cl)cc12] 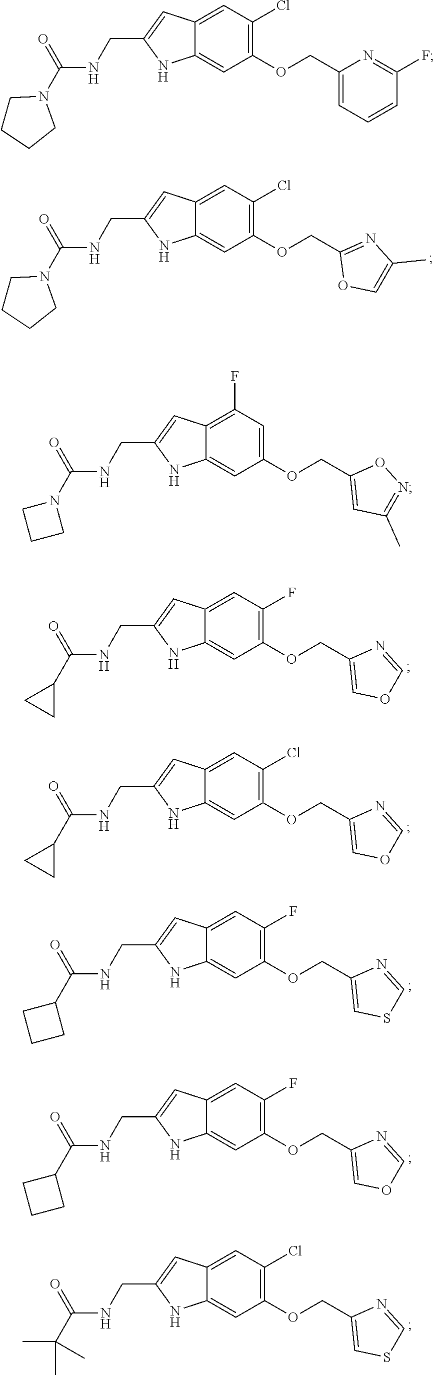 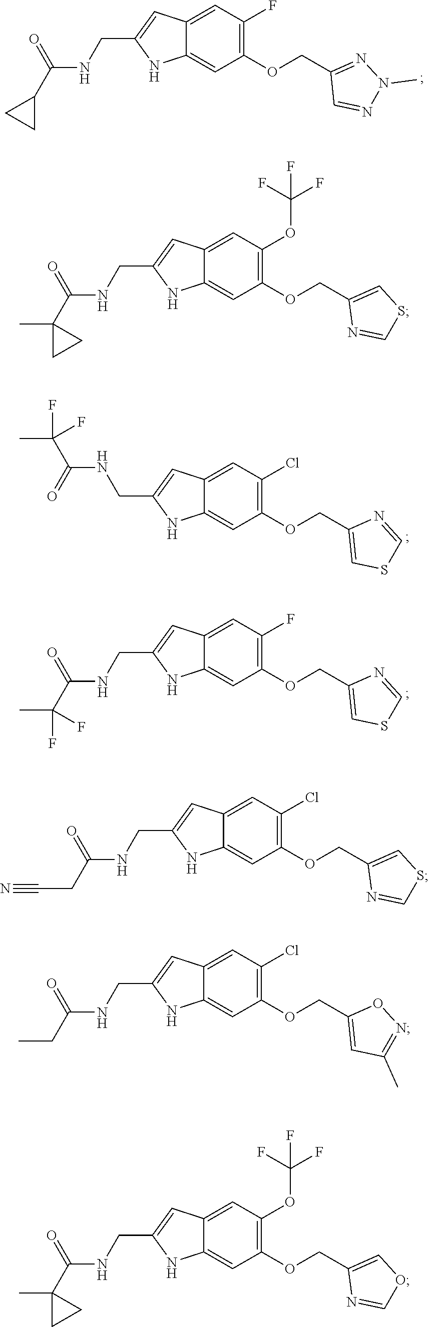 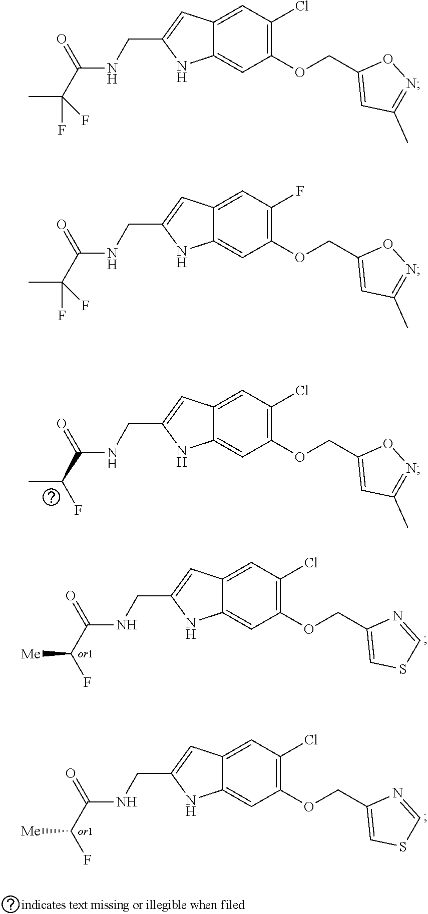 [SMILES: Cc1cc(COc2cc3[nH]c(CNC(=O)C(C)(F)F)cc3cc2Cl)on1 ; Cc1cc(COc2cc3[nH]c(CNC(=O)C(C)(F)F)cc3cc2F)on1 ; Cc1cc(COc2cc3[nH]c(CNC(=O)[C@H](C)F)cc3cc2Cl)on1 ; C[C@H](F)C(=O)NCc1cc2cc(Cl)c(OCc3cscn3)cc2[nH]1 ; C[C@@H](F)C(=O)NCc1cc2cc(Cl)c(OCc3cscn3)cc2[nH]1] 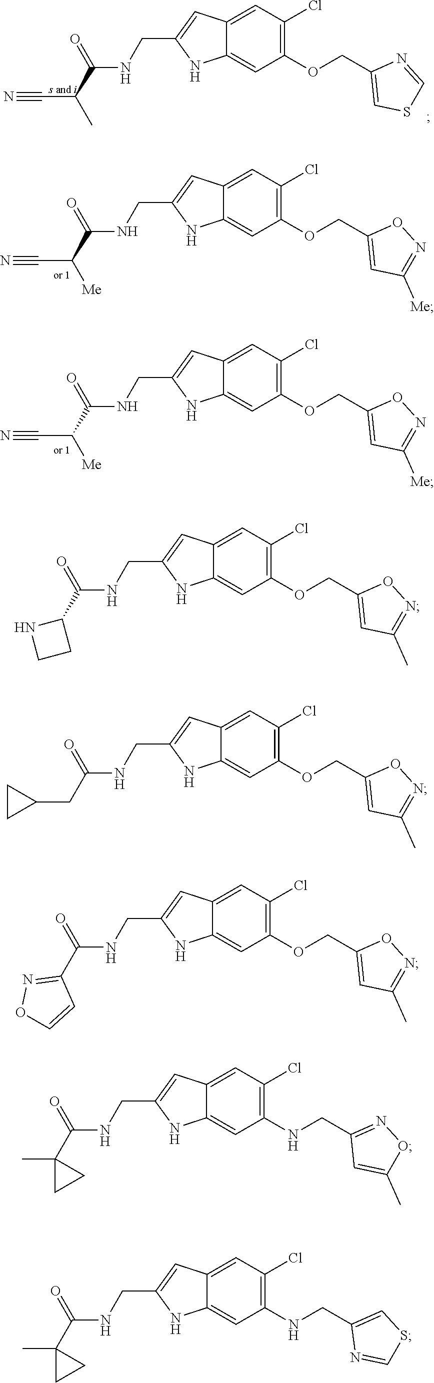 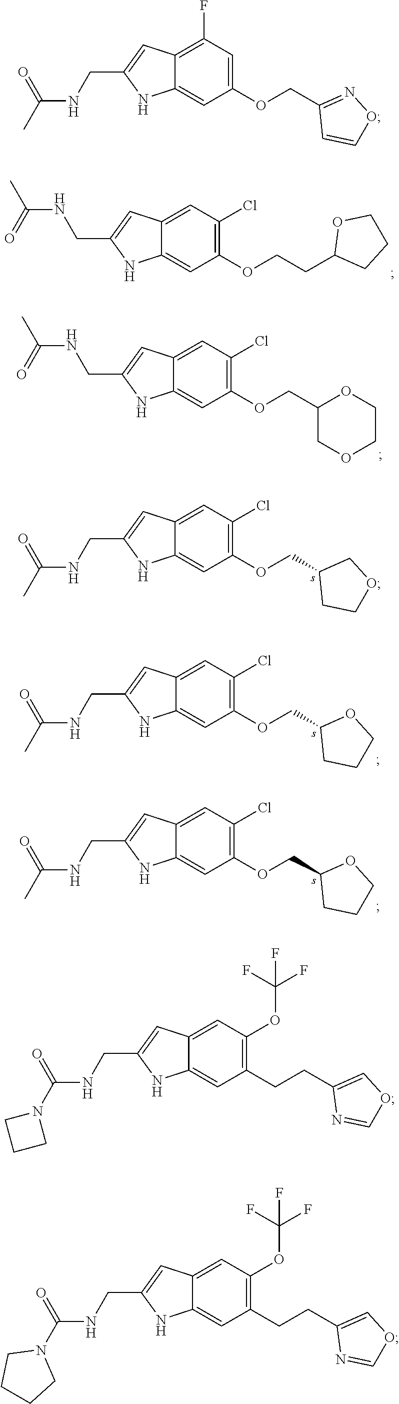 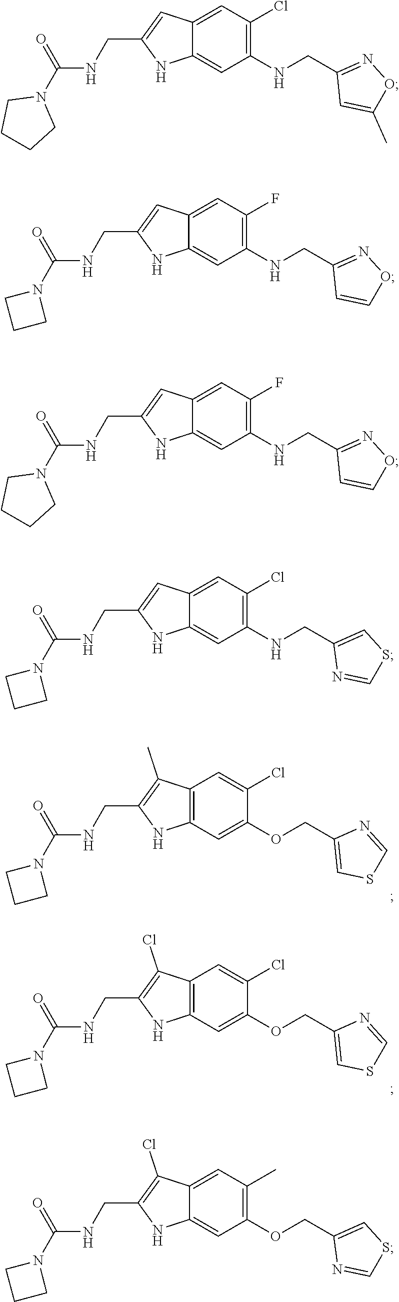 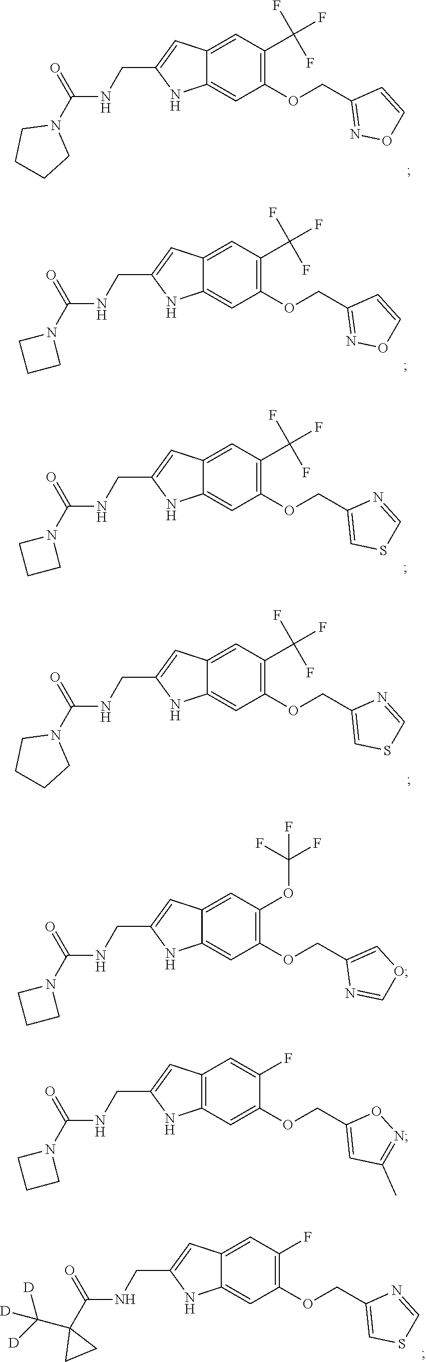 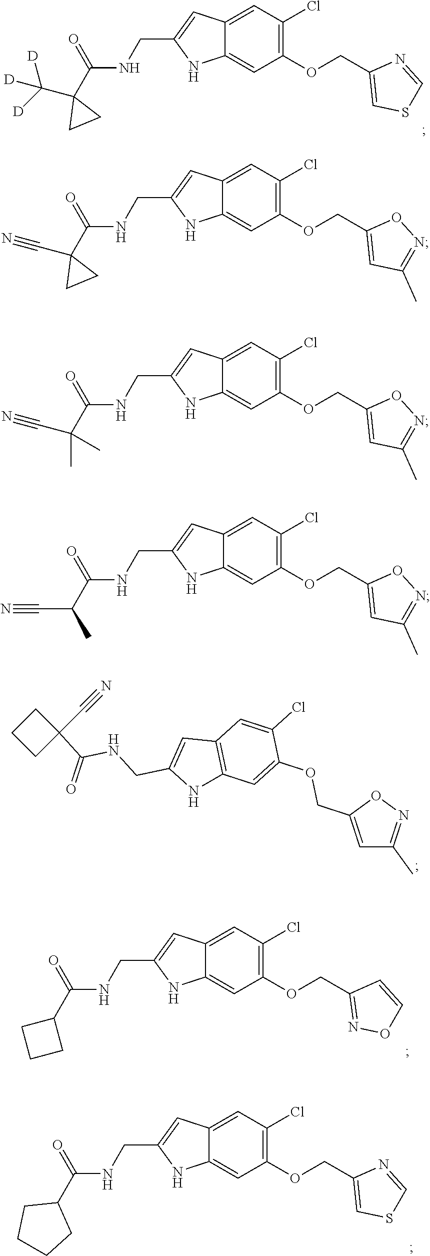 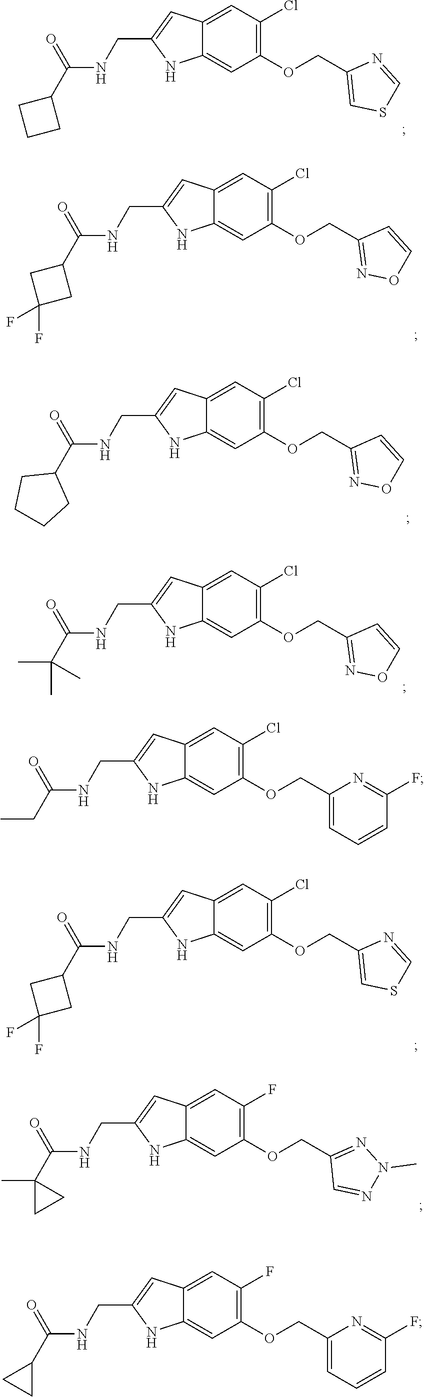 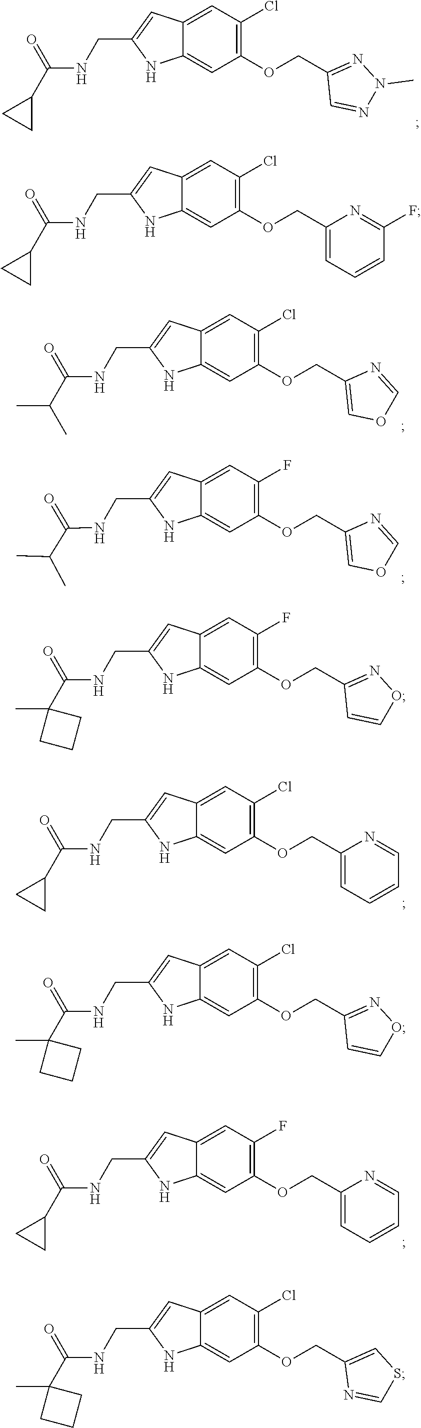 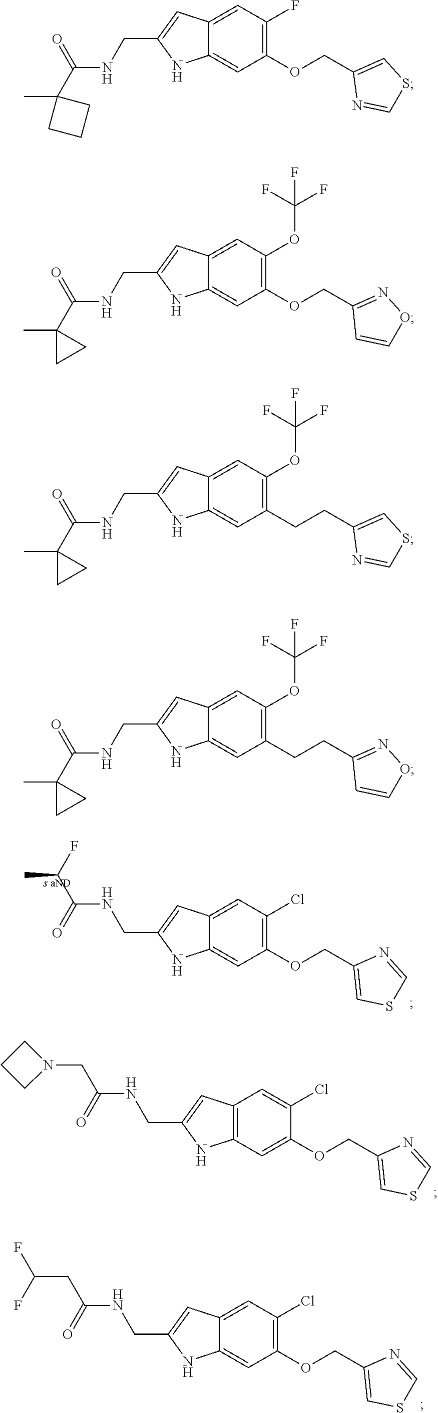 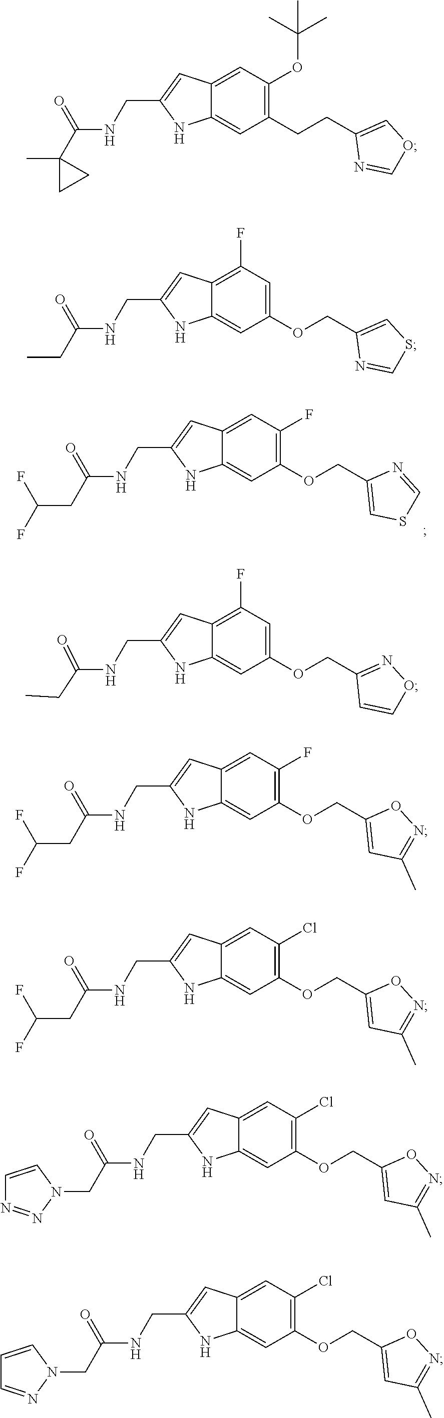 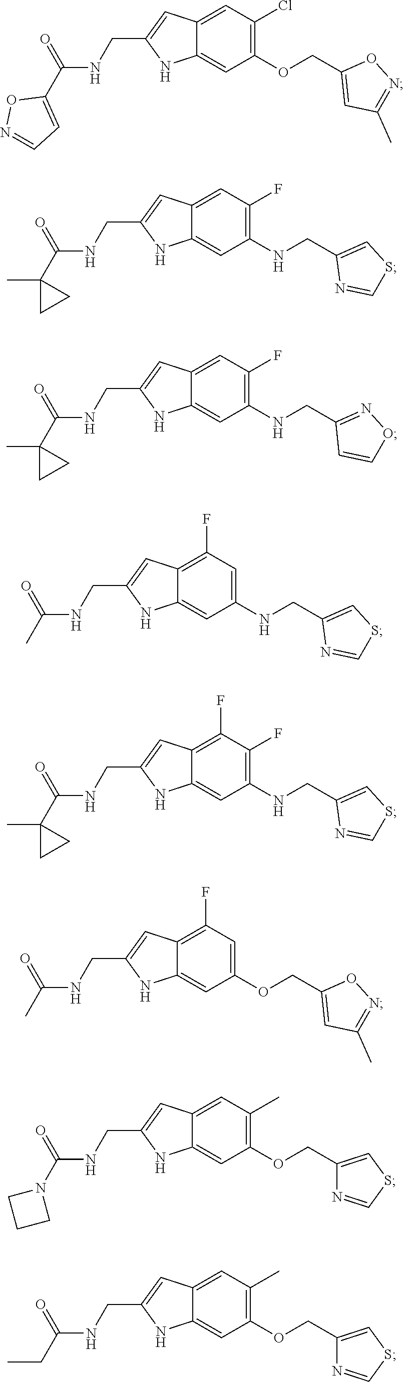 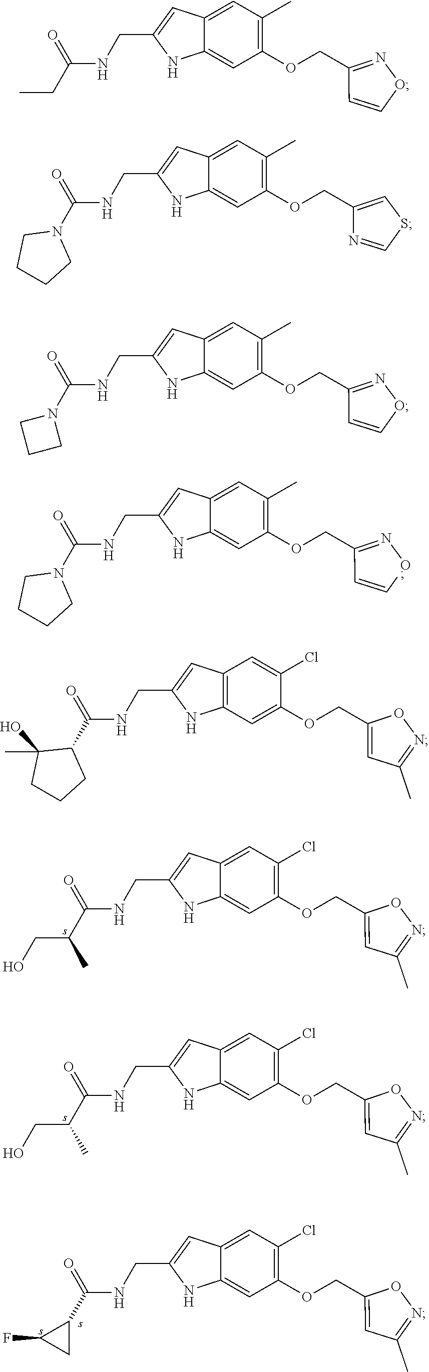 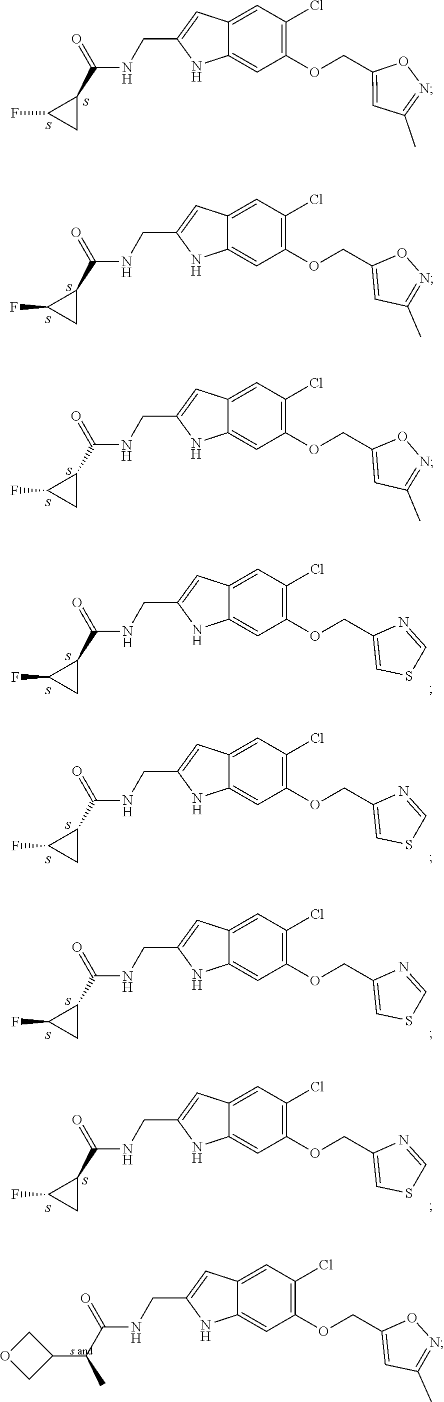 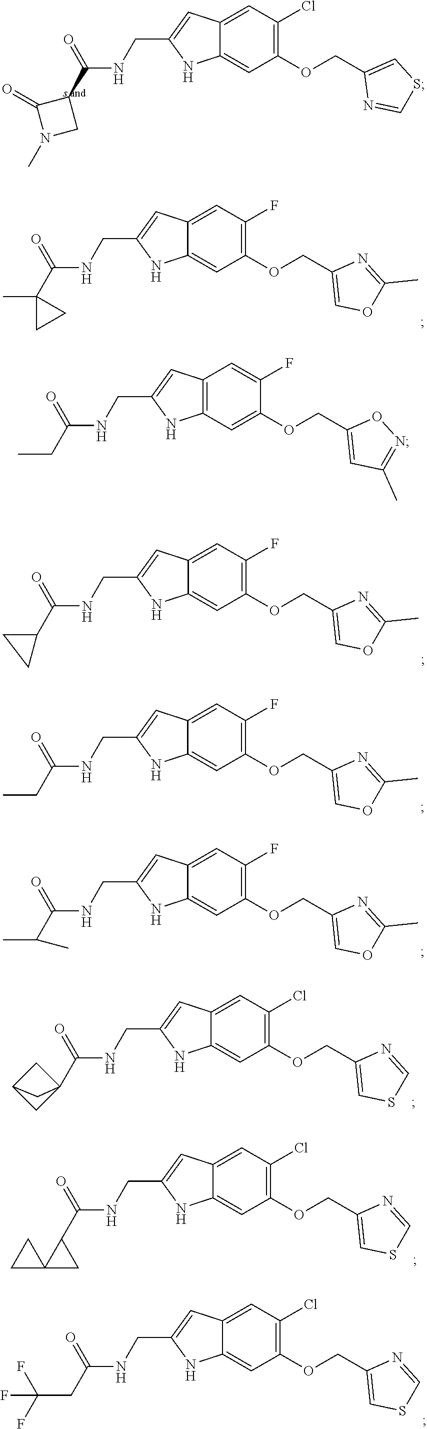 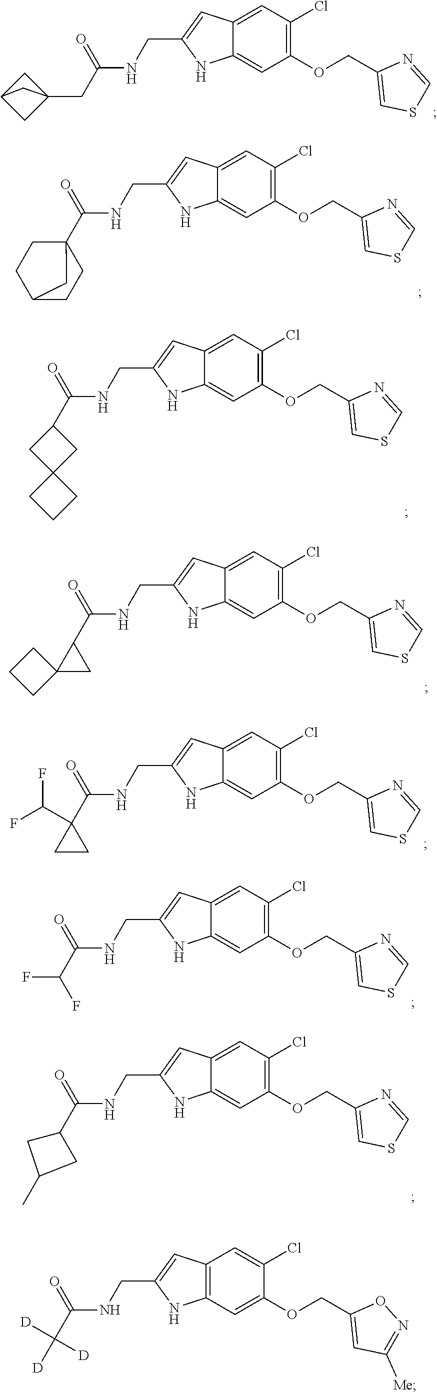 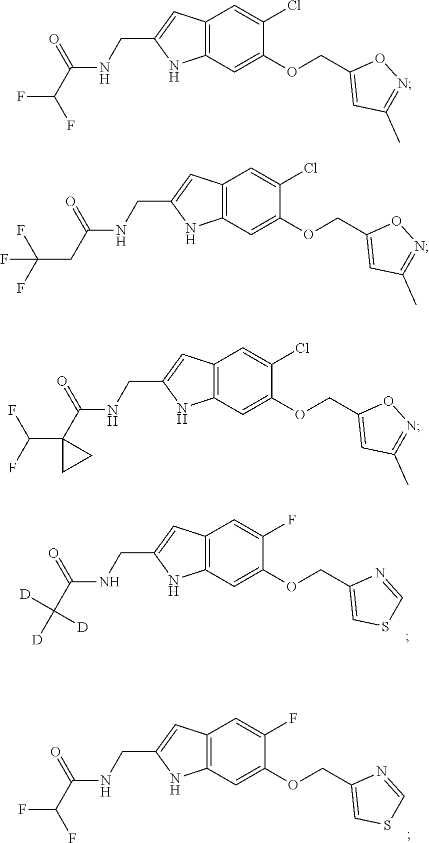 [SMILES: Cc1cc(COc2cc3[nH]c(CNC(=O)C(F)F)cc3cc2Cl)on1 ; Cc1cc(COc2cc3[nH]c(CNC(=O)CC(F)(F)F)cc3cc2Cl)on1 ; Cc1cc(COc2cc3[nH]c(CNC(=O)C4(C(F)F)CC4)cc3cc2Cl)on1 ; [2H]C([2H])([2H])C(=O)NCc1cc2cc(F)c(OCc3cscn3)cc2[nH]1 ; O=C(NCc1cc2cc(F)c(OCc3cscn3)cc2[nH]1)C(F)F] 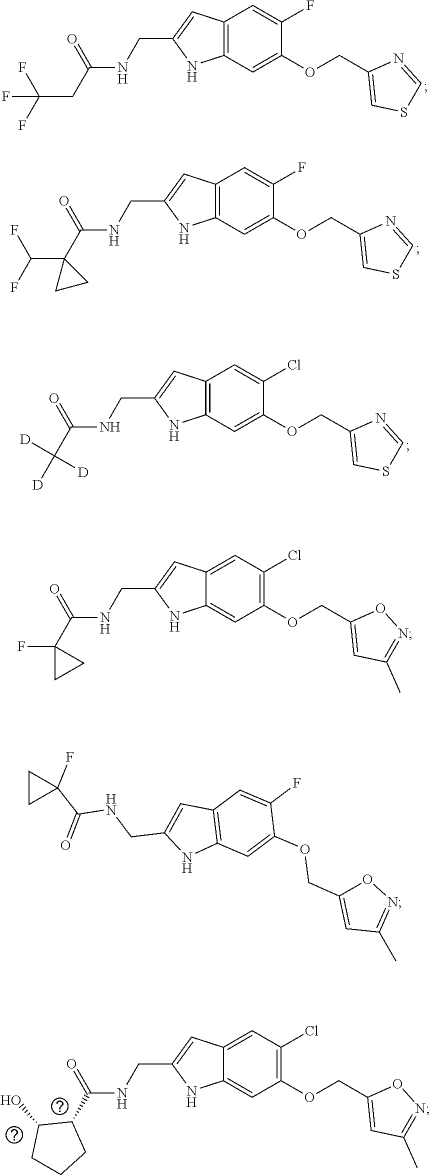 [SMILES: O=C(CC(F)(F)F)NCc1cc2cc(F)c(OCc3cscn3)cc2[nH]1 ; O=C(NCc1cc2cc(F)c(OCc3cscn3)cc2[nH]1)C1(C(F)F)CC1 ; [2H]C([2H])([2H])C(=O)NCc1cc2cc(Cl)c(OCc3cscn3)cc2[nH]1 ; Cc1cc(COc2cc3[nH]c(CNC(=O)C4(F)CC4)cc3cc2Cl)on1 ; Cc1cc(COc2cc3[nH]c(CNC(=O)C4(F)CC4)cc3cc2F)on1 ; Cc1cc(COc2cc3[nH]c(CNC(=O)[C@@H]4CCC[C@@H]4O)cc3cc2Cl)on1] 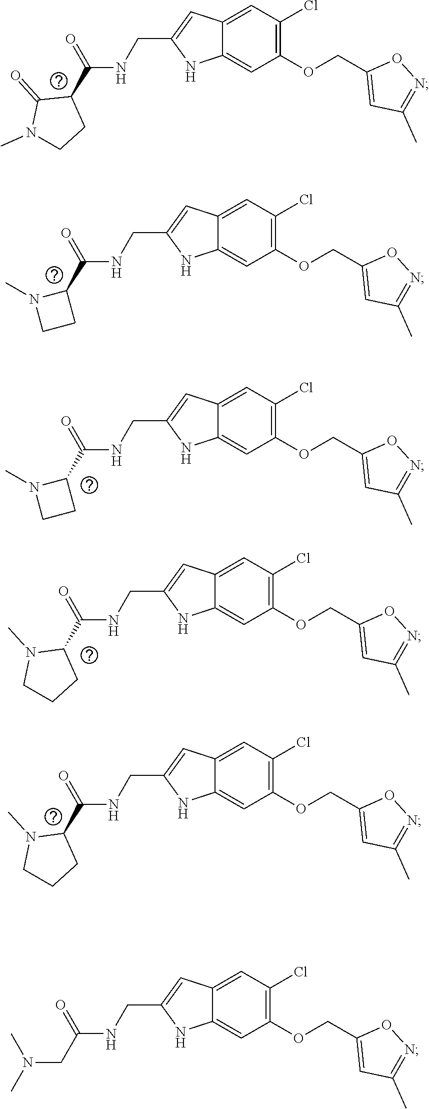 [SMILES: Cc1cc(COc2cc3[nH]c(CNC(=O)[C@H]4CCN(C)C4=O)cc3cc2Cl)on1 ; Cc1cc(COc2cc3[nH]c(CNC(=O)[C@H]4CCN4C)cc3cc2Cl)on1 ; Cc1cc(COc2cc3[nH]c(CNC(=O)[C@@H]4CCN4C)cc3cc2Cl)on1 ; Cc1cc(COc2cc3[nH]c(CNC(=O)[C@@H]4CCCN4C)cc3cc2Cl)on1 ; Cc1cc(COc2cc3[nH]c(CNC(=O)[C@H]4CCCN4C)cc3cc2Cl)on1 ; Cc1cc(COc2cc3[nH]c(CNC(=O)CN(C)C)cc3cc2Cl)on1] 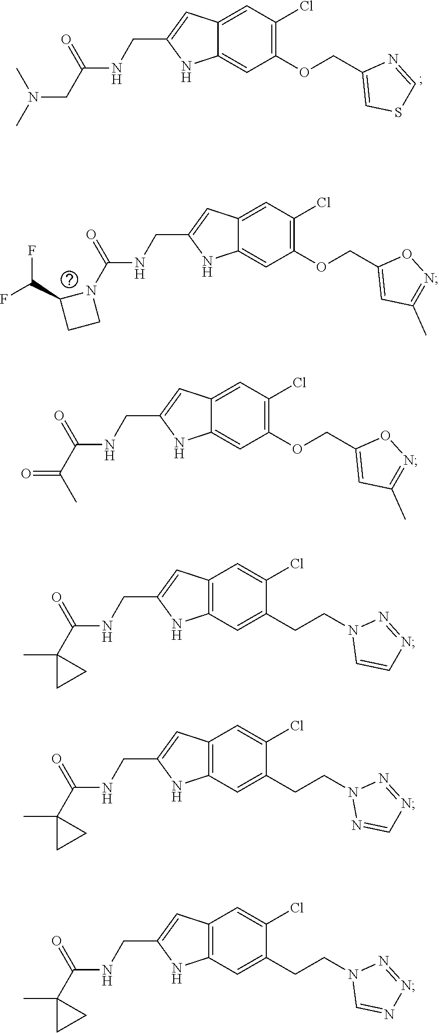 [SMILES: CN(C)CC(=O)NCc1cc2cc(Cl)c(OCc3cscn3)cc2[nH]1 ; Cc1cc(COc2cc3[nH]c(CNC(=O)N4CC[C@H]4C(F)F)cc3cc2Cl)on1 ; CC(=O)C(=O)NCc1cc2cc(Cl)c(OCc3cc(C)no3)cc2[nH]1 ; CC1(C(=O)NCc2cc3cc(Cl)c(CCn4ccnn4)cc3[nH]2)CC1 ; CC1(C(=O)NCc2cc3cc(Cl)c(CCn4ncnn4)cc3[nH]2)CC1 ; CC1(C(=O)NCc2cc3cc(Cl)c(CCn4cnnn4)cc3[nH]2)CC1] 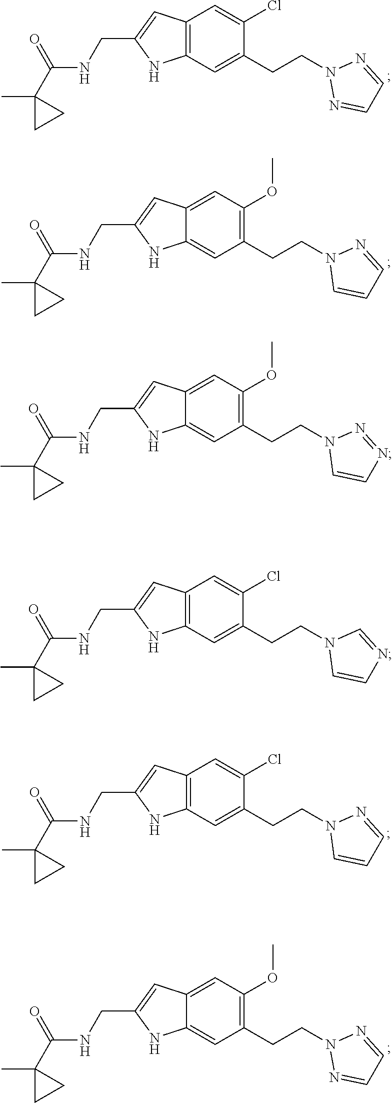 [SMILES: CC1(C(=O)NCc2cc3cc(Cl)c(CCn4nccn4)cc3[nH]2)CC1 ; COc1cc2cc(CNC(=O)C3(C)CC3)[nH]c2cc1CCn1cccn1 ; COc1cc2cc(CNC(=O)C3(C)CC3)[nH]c2cc1CCn1ccnn1 ; CC1(C(=O)NCc2cc3cc(Cl)c(CCn4ccnc4)cc3[nH]2)CC1 ; CC1(C(=O)NCc2cc3cc(Cl)c(CCn4cccn4)cc3[nH]2)CC1 ; COc1cc2cc(CNC(=O)C3(C)CC3)[nH]c2cc1CCn1nccn1] 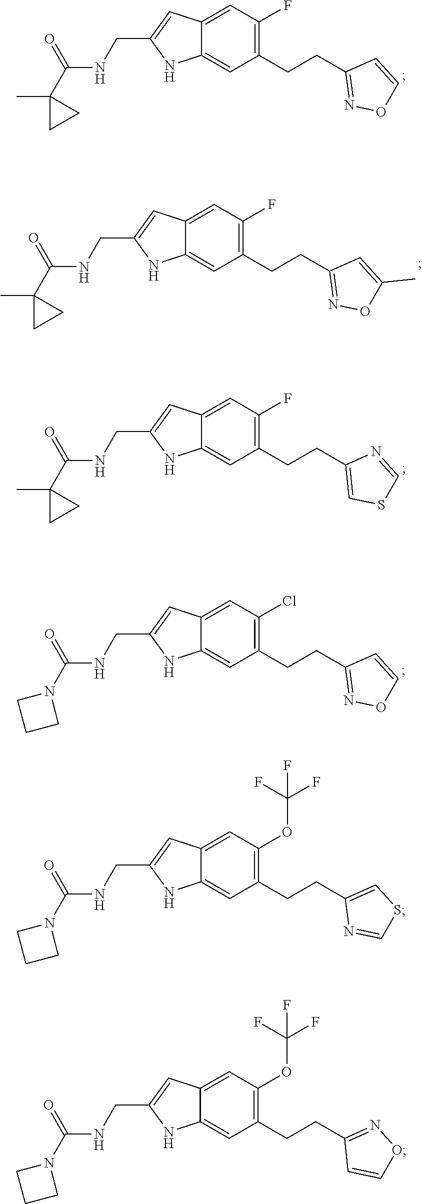 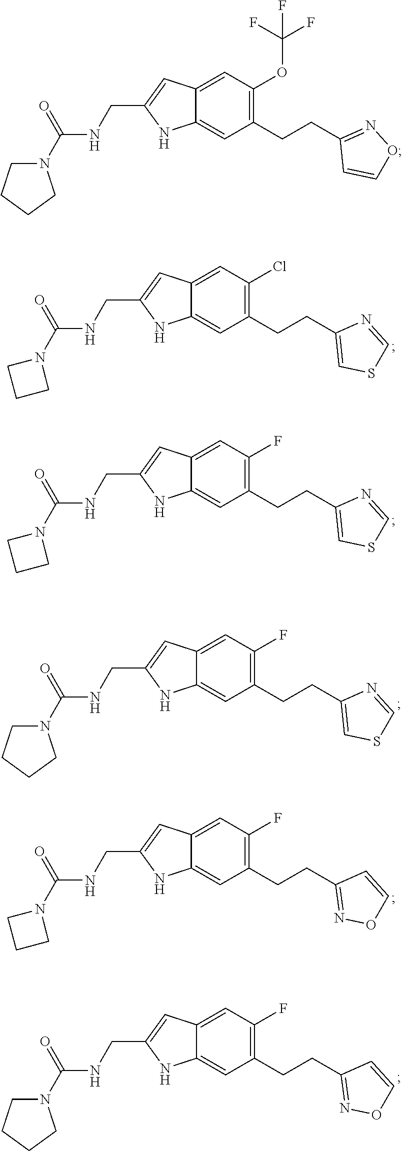 [SMILES: O=C(NCc1cc2cc(OC(F)(F)F)c(CCc3ccon3)cc2[nH]1)N1CCCC1 ; O=C(NCc1cc2cc(Cl)c(CCc3cscn3)cc2[nH]1)N1CCC1 ; O=C(NCc1cc2cc(F)c(CCc3cscn3)cc2[nH]1)N1CCC1 ; O=C(NCc1cc2cc(F)c(CCc3cscn3)cc2[nH]1)N1CCCC1 ; O=C(NCc1cc2cc(F)c(CCc3ccon3)cc2[nH]1)N1CCC1 ; O=C(NCc1cc2cc(F)c(CCc3ccon3)cc2[nH]1)N1CCCC1] 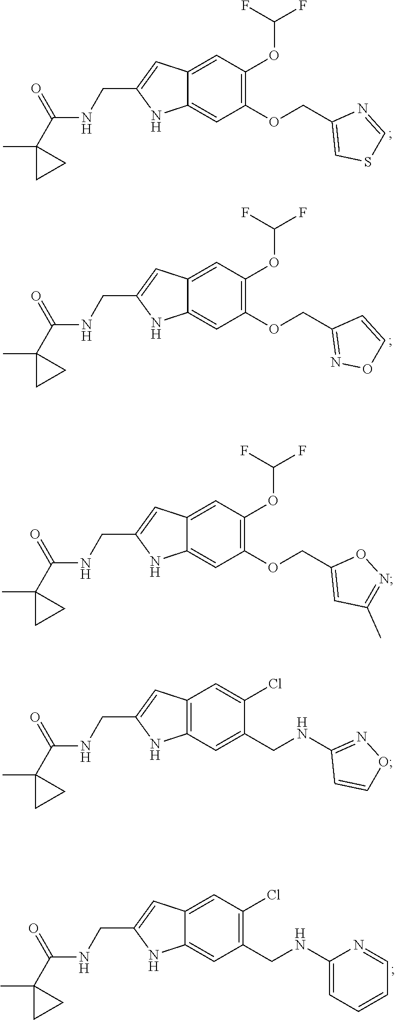 [SMILES: CC1(C(=O)NCc2cc3cc(OC(F)F)c(OCc4cscn4)cc3[nH]2)CC1 ; CC1(C(=O)NCc2cc3cc(OC(F)F)c(OCc4ccon4)cc3[nH]2)CC1 ; Cc1cc(COc2cc3[nH]c(CNC(=O)C4(C)CC4)cc3cc2OC(F)F)on1 ; CC1(C(=O)NCc2cc3cc(Cl)c(CNc4ccon4)cc3[nH]2)CC1 ; CC1(C(=O)NCc2cc3cc(Cl)c(CNc4ccccn4)cc3[nH]2)CC1] 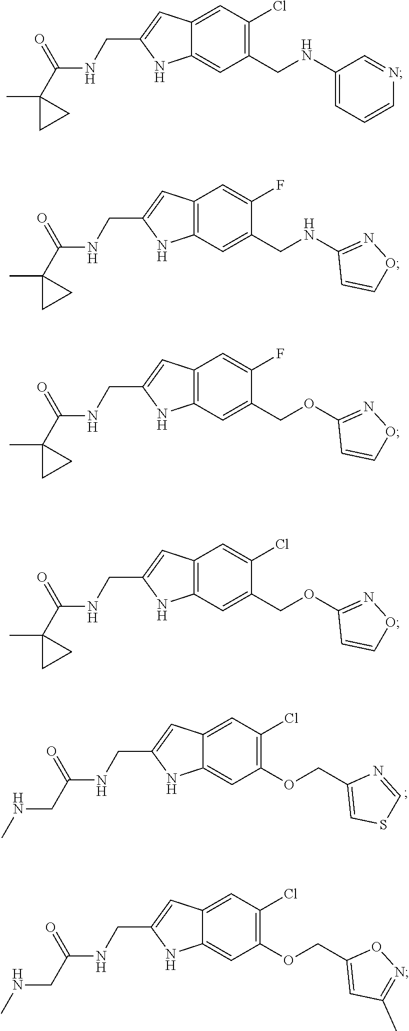 [SMILES: CC1(C(=O)NCc2cc3cc(Cl)c(CNc4cccnc4)cc3[nH]2)CC1 ; CC1(C(=O)NCc2cc3cc(F)c(CNc4ccon4)cc3[nH]2)CC1 ; CC1(C(=O)NCc2cc3cc(F)c(COc4ccon4)cc3[nH]2)CC1 ; CC1(C(=O)NCc2cc3cc(Cl)c(COc4ccon4)cc3[nH]2)CC1 ; CNCC(=O)NCc1cc2cc(Cl)c(OCc3cscn3)cc2[nH]1 ; CNCC(=O)NCc1cc2cc(Cl)c(OCc3cc(C)no3)cc2[nH]1] 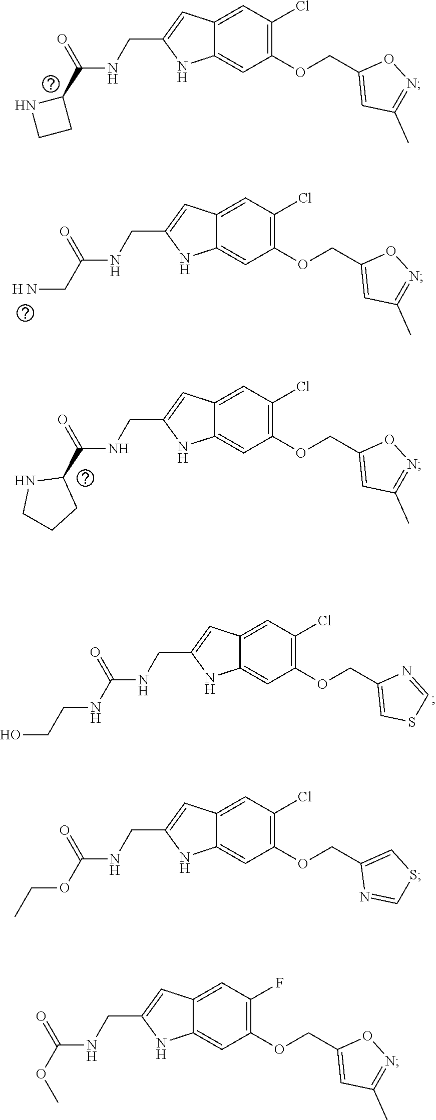 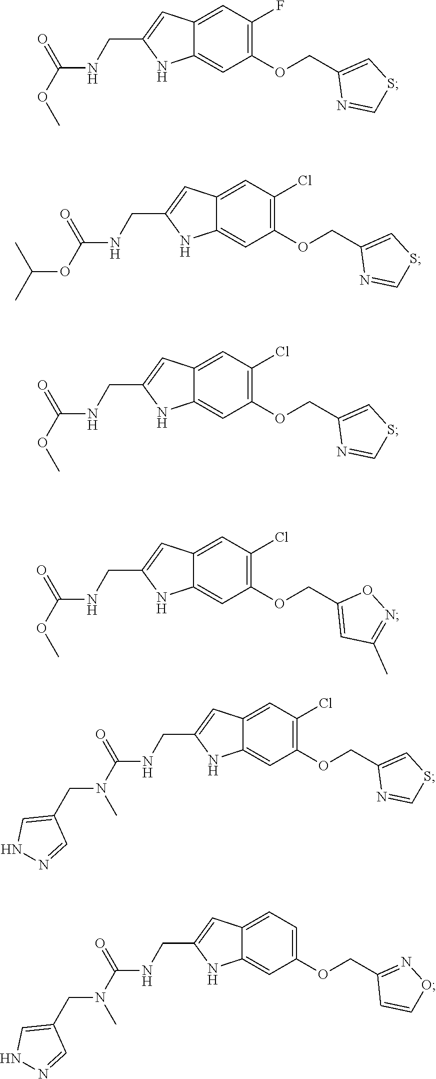 [SMILES: COC(=O)NCc1cc2cc(F)c(OCc3cscn3)cc2[nH]1 ; CC(C)OC(=O)NCc1cc2cc(Cl)c(OCc3cscn3)cc2[nH]1 ; COC(=O)NCc1cc2cc(Cl)c(OCc3cscn3)cc2[nH]1 ; COC(=O)NCc1cc2cc(Cl)c(OCc3cc(C)no3)cc2[nH]1 ; CN(Cc1cn[nH]c1)C(=O)NCc1cc2cc(Cl)c(OCc3cscn3)cc2[nH]1 ; CN(Cc1cn[nH]c1)C(=O)NCc1cc2ccc(OCc3ccon3)cc2[nH]1] 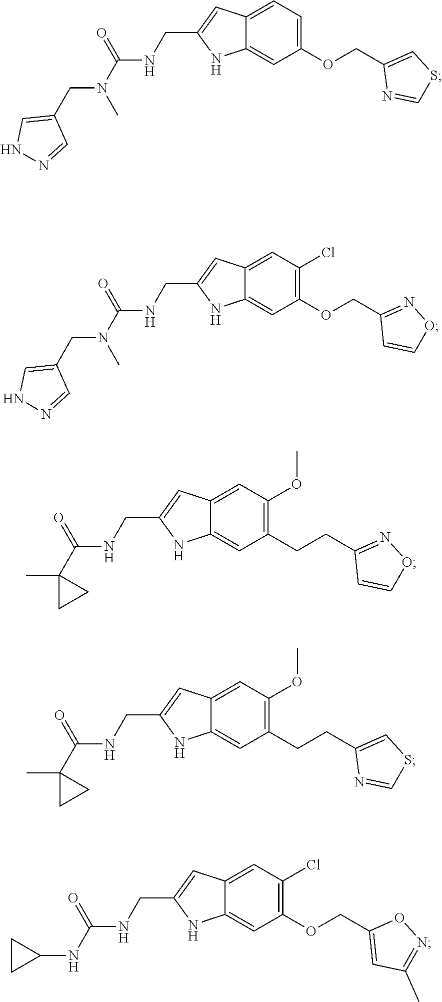 [SMILES: CN(Cc1cn[nH]c1)C(=O)NCc1cc2ccc(OCc3cscn3)cc2[nH]1 ; CN(Cc1cn[nH]c1)C(=O)NCc1cc2cc(Cl)c(OCc3ccon3)cc2[nH]1 ; COc1cc2cc(CNC(=O)C3(C)CC3)[nH]c2cc1CCc1ccon1 ; COc1cc2cc(CNC(=O)C3(C)CC3)[nH]c2cc1CCc1cscn1 ; Cc1cc(COc2cc3[nH]c(CNC(=O)NC4CC4)cc3cc2Cl)on1] 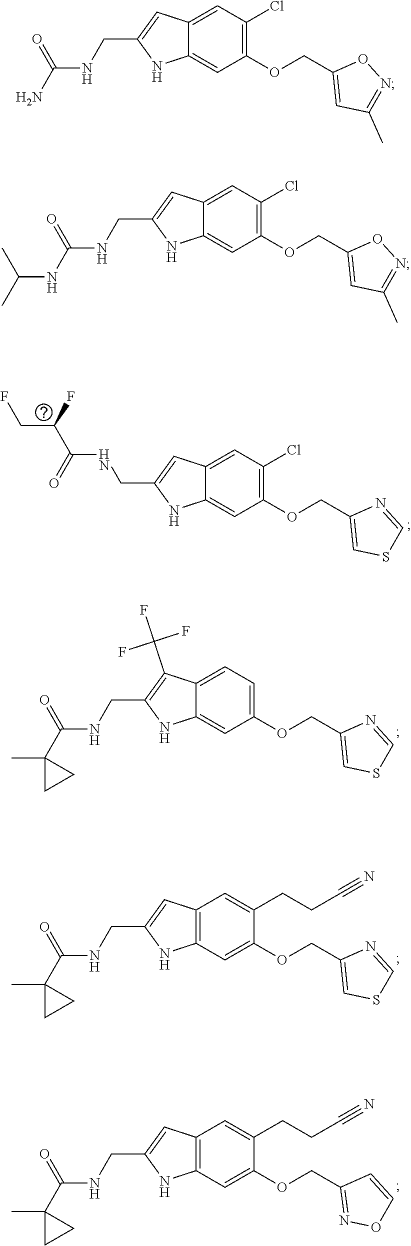 [SMILES: Cc1cc(COc2cc3[nH]c(CNC(N)=O)cc3cc2Cl)on1 ; Cc1cc(COc2cc3[nH]c(CNC(=O)NC(C)C)cc3cc2Cl)on1 ; O=C(NCc1cc2cc(Cl)c(OCc3cscn3)cc2[nH]1)[C@H](F)CF ; CC1(C(=O)NCc2[nH]c3cc(OCc4cscn4)ccc3c2C(F)(F)F)CC1 ; CC1(C(=O)NCc2cc3cc(CCC#N)c(OCc4cscn4)cc3[nH]2)CC1 ; CC1(C(=O)NCc2cc3cc(CCC#N)c(OCc4ccon4)cc3[nH]2)CC1] 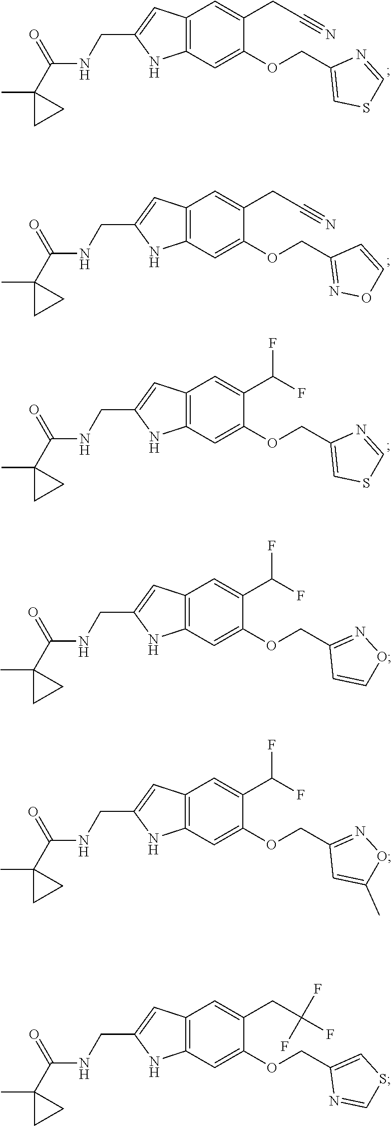 [SMILES: CC1(C(=O)NCc2cc3cc(CC#N)c(OCc4cscn4)cc3[nH]2)CC1 ; CC1(C(=O)NCc2cc3cc(CC#N)c(OCc4ccon4)cc3[nH]2)CC1 ; CC1(C(=O)NCc2cc3cc(C(F)F)c(OCc4cscn4)cc3[nH]2)CC1 ; CC1(C(=O)NCc2cc3cc(C(F)F)c(OCc4ccon4)cc3[nH]2)CC1 ; Cc1cc(COc2cc3[nH]c(CNC(=O)C4(C)CC4)cc3cc2C(F)F)no1 ; CC1(C(=O)NCc2cc3cc(CC(F)(F)F)c(OCc4cscn4)cc3[nH]2)CC1] 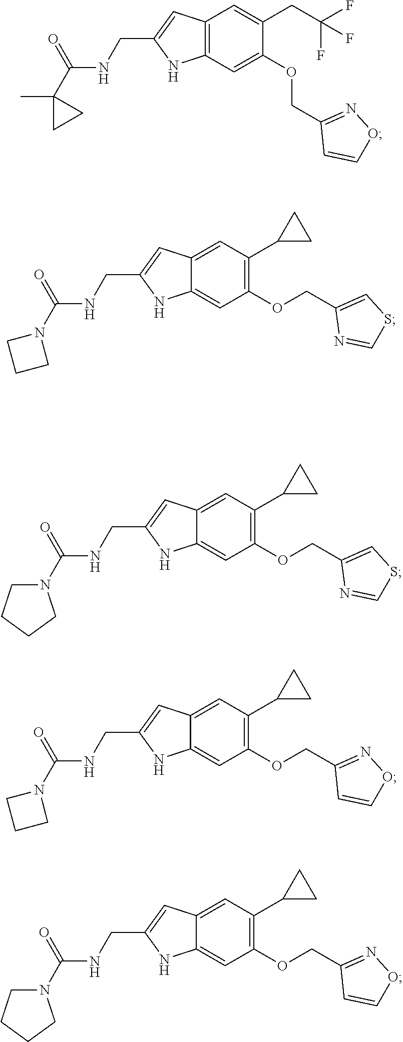 [SMILES: CC1(C(=O)NCc2cc3cc(CC(F)(F)F)c(OCc4ccon4)cc3[nH]2)CC1 ; O=C(NCc1cc2cc(C3CC3)c(OCc3cscn3)cc2[nH]1)N1CCC1 ; O=C(NCc1cc2cc(C3CC3)c(OCc3cscn3)cc2[nH]1)N1CCCC1 ; O=C(NCc1cc2cc(C3CC3)c(OCc3ccon3)cc2[nH]1)N1CCC1 ; O=C(NCc1cc2cc(C3CC3)c(OCc3ccon3)cc2[nH]1)N1CCCC1] 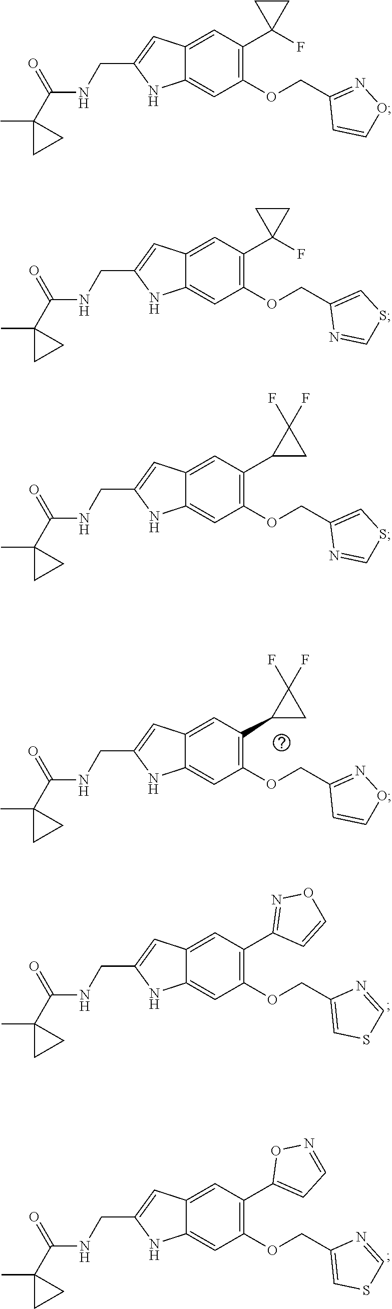 [SMILES: CC1(C(=O)NCc2cc3cc(C4(F)CC4)c(OCc4ccon4)cc3[nH]2)CC1 ; CC1(C(=O)NCc2cc3cc(C4(F)CC4)c(OCc4cscn4)cc3[nH]2)CC1 ; CC1(C(=O)NCc2cc3cc(C4CC4(F)F)c(OCc4cscn4)cc3[nH]2)CC1 ; CC1(C(=O)NCc2cc3cc([C@@H]4CC4(F)F)c(OCc4ccon4)cc3[nH]2)CC1 ; CC1(C(=O)NCc2cc3cc(-c4ccon4)c(OCc4cscn4)cc3[nH]2)CC1 ; CC1(C(=O)NCc2cc3cc(-c4ccno4)c(OCc4cscn4)cc3[nH]2)CC1] 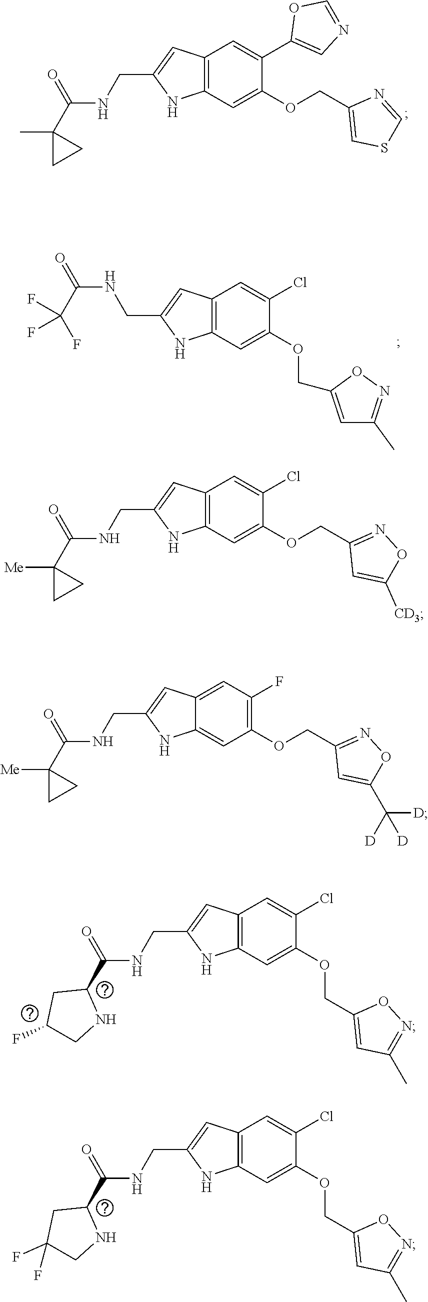 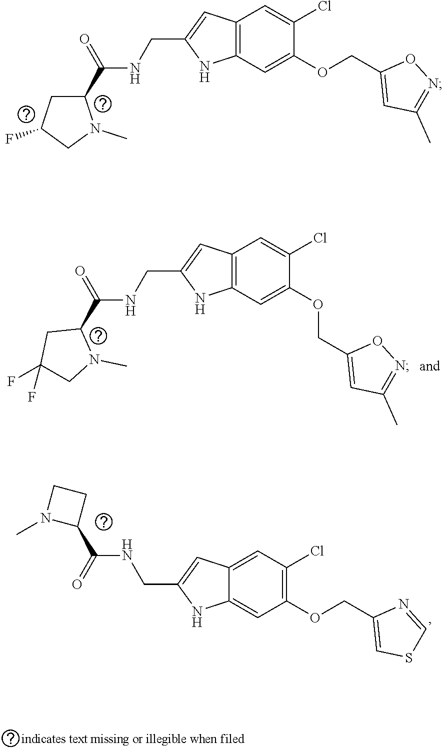 [SMILES: Cc1cc(COc2cc3[nH]c(CNC(=O)[C@@H]4C[C@@H](F)CN4C)cc3cc2Cl)on1 ; Cc1cc(COc2cc3[nH]c(CNC(=O)[C@@H]4CC(F)(F)CN4C)cc3cc2Cl)on1 ; CN1CC[C@H]1C(=O)NCc1cc2cc(Cl)c(OCc3cscn3)cc2[nH]1]
or a stereoisomer or tautomer thereof, or a pharmaceutically acceptable salt of any of the foregoing.

65. A process for preparing a compound of formula (I′): 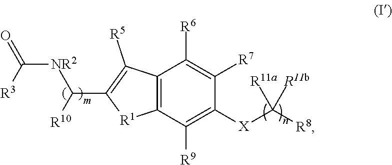
or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy,
wherein the process comprises:
(a) reacting a compound of formula (I′-1): 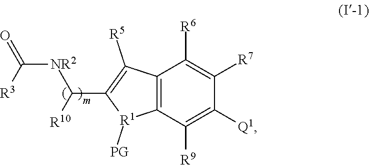
or a stereoisomer or tautomer thereof, or a pharmaceutically acceptable salt of any of the foregoing, wherein:
Q¹ is halo, boronic acid, or boronic ester;
PG is absent, or a protecting group;
m is an integer from 1 to 4;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H, or halo;
R¹⁰ is H; and
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
with a compound of formula (I′-2): 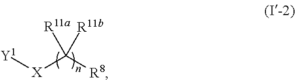
or a stereoisomer or tautomer thereof, or a pharmaceutically acceptable salt of any of the foregoing, wherein:
Y¹ is H, hydrazone, boronic acid, boronic ester, or halo;
n is an integer from 0 to 4;
X is —O—, —S—, —C(R^{12a})(R^{12b})—, —N(H)—, or —N(C₁₋₆alkyl)-;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
in the presence of one or more coupling reagent, to provide a compound of formula (I′-3): 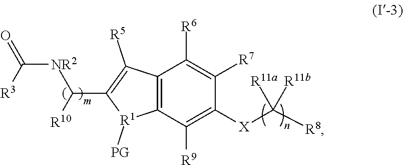
or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
followed by,
(b) optionally contacting the compound of formula (I′-3) with a deprotecting agent;
to provide a compound of formula (I′).

66-68. (canceled)

69. A process for preparing a compound of formula (I′): 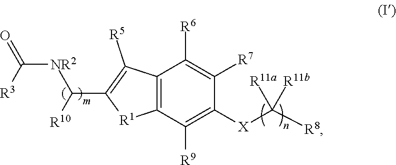
or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆ haloalkyl, wherein
the C₁₋₆alkyl of R⁵ and R⁷ are each independently optionally substituted with one or more halo or CN;
R⁶ and R⁹ are each independently H or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy,
wherein the process comprises:
(a) reacting a compound of formula (IV′-1): 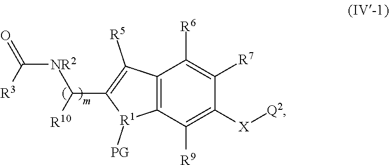
or a stereoisomer or tautomer thereof, or a pharmaceutically acceptable salt of any of the foregoing, wherein:
PG is absent or a protecting group;
Q² is H, or (═O);
m is an integer from 1 to 4;
X is —O—, —S—, —C(R^{12a})(R^{12b})—, —N(H)—, or —N(C₁₋₆alkyl)-;
R¹ is —N—, —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H, or halo;
R¹⁰ is H;
R^{12a} and R^{12b} are each independently, H, or C₁₋₆alkyl; and
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
with a compound of formula (II′-2): 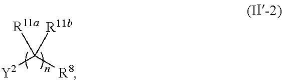
or a stereoisomer or tautomer thereof, or a pharmaceutically acceptable salt of any of the foregoing, wherein:
Y² is OH, (═O), halo, triphenylphosphonium salt, or sulfonic ester;
n is an integer from 0 to 4; R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R^{11a} and R^{11b} are each independently at each occurrence, H, or C₁₋₆alkyl; and
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
in the presence of one or more coupling reagents, to provide a compound of formula (I′-3): 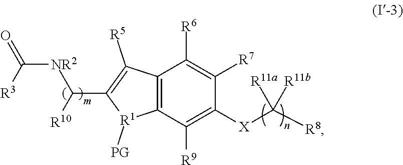
or a stereoisomer or tautomer thereof, or a pharmaceutically acceptable salt of any of the foregoing, wherein:
PG is absent or a protecting group;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —N—NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl;
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
followed by,
(b) optionally contacting the compound of formula (I′-3) with a deprotecting agent;
to provide a compound of formula (I′).

70-74. (canceled)

75. A process for preparing a compound of formula (I′): 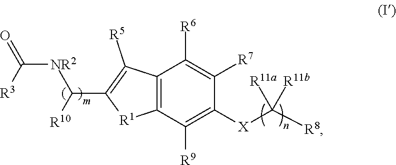
or a stereoisomer or tautomer thereof, or a pharmaceutically acceptable salt of any of the foregoing, wherein:
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a};
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H or halo;
R⁸ is —C(O)N(R₄)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H or C₁₋₆alkyl, or
one of R^{11a} and R^{11b} is H or C₁₋₆alkyl, and the other of R^{11a} and R^{11b} is taken together with one of R^{12a} or R^{12b} to form a C₃₋₁₀cycloalkyl;
R^{12a} and R^{12b} are each independently, H or C₁₋₆alkyl, or
one of R^{12a} and R^{12b} is H or C₁₋₆alkyl, and the other of R^{12a} and R^{12b} is taken together with one of R^{11a} or R^{11b} to form a C₃₋₁₀cycloalkyl;
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy,
wherein the process comprises:
reacting a compound of formula (III′-1): 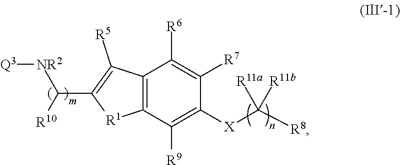
or a stereoisomer or tautomer thereof, or a pharmaceutically acceptable salt of any of the foregoing, wherein:
Q³ is H;
m is an integer from 1 to 4;
n is an integer from 0 to 4;
X is —O—, —S—, —C(R^{12a})(R^{12b})—, —N(H)—, or —N(C₁₋₆alkyl)-;
R¹ is —NH—, —O—, or —S—;
R² is H;
each R⁴ is independently H, C₁₋₆alkyl, C₁₋₆haloalkyl, or C₃₋₁₀cycloalkyl, or
both R⁴ are taken together with the N atom to which they are attached to form a 4-10 membered heterocyclyl optionally substituted with one or more R^{a};
R⁵ and R⁷ are each independently H, halo, C₁₋₆alkyl, C₁₋₆alkoxy, C₃₋₁₀cycloalkyl, or C₁₋₆haloalkyl, wherein
the C₁₋₆alkyl of R⁵ and R⁷ are each independently optionally substituted with one or more halo or CN;
R⁶ and R⁹ are each independently H, or halo;
R⁸ is —C(O)N(R⁴)₂, C₃₋₁₀cycloalkyl, C₆₋₂₀aryl, 3-10 membered heterocyclyl, or 5-20 membered heteroaryl, wherein each R⁸ is optionally substituted with one or more R^{a};
R¹⁰ is H;
R^{11a} and R^{11b} are each independently at each occurrence, H, or C₁₋₆alkyl, or taken together with one of R^{12a} or R^{12b} to form a C₃₋₁₀cycloalkyl;
R^{12a} and R^{12b} are each independently, H, or C₁₋₆alkyl, or
one of R^{12a} and R^{12b} is H, or C₁₋₆alkyl, and the other of R^{12a} and R^{12b} is taken together with R^{11a} or R^{11b} to form a C₃₋₁₀cycloalkyl; and
each R^{a} is independently —OH, oxo, —CN, halo, C₁₋₆alkyl, C₁₋₆alkoxy, C₁₋₆haloalkyl, —O—C₁₋₆haloalkyl, —NH₂, —NH(C₁₋₆alkyl), —N(C₁₋₆alkyl)₂, —C(O)NH₂, —C(O)NH(C₁₋₆alkyl), —C(O)N(C₁₋₆alkyl)₂, 4-10 membered heterocyclyl, C₃₋₁₀cycloalkyl, or —O—C₃₋₁₀cycloalkyl, wherein
the C₁₋₆alkyl, C₁₋₆alkoxy, 4-10 membered heterocyclyl, C₃₋₁₀cycloalkyl, or —O—C₃₋₁₀cycloalkyl, of R^{a} is optionally substituted with one or more R^{b}; and
each R^{b} is independently D, —OH, halo, C₁₋₆alkyl, or C₁₋₆alkoxy;
with a compound of formula (III′-2): 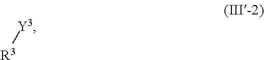
or a stereoisomer or tautomer thereof, or a pharmaceutically acceptable salt of any of the foregoing, wherein:
Y³ is —C(O)OH, or —C(O)-halo;
R³ is C₁₋₆alkyl, C₁₋₆haloalkyl, C₃₋₁₀cycloalkyl, —N(R⁴)₂, or 4-10 membered heterocyclyl, wherein each R³ is optionally substituted with one or more R^{a}; and
each R^{a} is, independently at each occurrence, —OH, halo, C₁₋₆alkyl, C₁₋₆haloalkyl, or C₃₋₁₀cycloalkyl;
in the presence of one or more coupling reagent, to provide a compound of formula (I′).

76. (canceled)

77. A pharmaceutical composition comprising (i) a compound of claim 1, or a stereoisomer or tautomer thereof, or a pharmaceutically acceptable salt of any of the foregoing, and (ii) one or more pharmaceutically acceptable excipients.

78. A method of modulating SLC6A19 in a cell, comprising exposing the cell to a an effective amount of
a) a compound of claim 1, or a stereoisomer or tautomer thereof, or a pharmaceutically acceptable salt of any of the foregoing, or
b) a pharmaceutical composition comprising (i) a compound of claim 1, or a stereoisomer or tautomer thereof, or a pharmaceutically acceptable salt of any of the foregoing, and (ii) one or more pharmaceutically acceptable excipients.

79. A method of inhibiting SLC6A19 in a cell, comprising exposing the cell to an effective amount of
a) a compound of claim 1, or a stereoisomer or tautomer thereof, or a pharmaceutically acceptable salt of any of the foregoing, or
b) a pharmaceutical composition comprising (i) a compound of claim 1, or a stereoisomer or tautomer thereof, or a pharmaceutically acceptable salt of any of the foregoing, and (ii) one or more pharmaceutically acceptable excipients.

80. A method of reducing systemic phenylalanine, tyrosine, glutamine, or glycine levels in an individual in need thereof, comprising administering to the individual an effective amount of
a) a compound of claim 1, or a stereoisomer or tautomer thereof, or a pharmaceutically acceptable salt of any of the foregoing, or
b) a pharmaceutical composition comprising (i) a compound of claim 1, or a stereoisomer or tautomer thereof, or a pharmaceutically acceptable salt of any of the foregoing, and (ii) one or more pharmaceutically acceptable excipients.

81. A method of treating a SLC6A19-mediated disease, disorder, or condition in an individual in need thereof, comprising administering to the individual,
a) a compound of claim 1, or a stereoisomer or tautomer thereof, or a pharmaceutically acceptable salt of any of the foregoing, or
b) a pharmaceutical composition comprising (i) a compound of claim 1, or a stereoisomer or tautomer thereof, or a pharmaceutically acceptable salt of any of the foregoing, and (ii) one or more pharmaceutically acceptable excipients.

82-87. (canceled)

88. A kit, comprising:
(i) a compound of claim 1, or a stereoisomer or tautomer thereof, or a pharmaceutically acceptable salt of any of the foregoing, or a pharmaceutical composition comprising a compound of claim 1, or a stereoisomer or tautomer thereof, or a pharmaceutically acceptable salt of any of the foregoing, and one or more pharmaceutically acceptable excipients and
(ii) instructions for use in treating an SLC6A19-mediated disease, disorder, or condition in an individual in need thereof.

89-100. (canceled)
